(19) United States
(12) Patent Application Publication
(10) Pub. No.: US 2024/0018115 A1
(43) Pub. Date: Jan. 18, 2024

(54) ALLOSTERIC CHROMENONE INHIBITORS OF PHOSPHOINOSITIDE 3-KINASE (PI3K) FOR THE TREATMENT OF DISEASE

(71) Applicant: Petra Pharma Corporation, New York, NY (US)

(72) Inventors: Sean Douglas ARONOW, Boulder, CO (US); Carina Ivonne JETTE, Pasadena, CA (US); Gabrielle R. KOLAKOWSKI, Durango, CO (US); Christopher Glenn MAYNE, Boulder, CO (US); Hua-Yu WANG, Chula Vista, CA (US); Michael Brian WELCH, Westminster, CO (US); Nathan Edward WRIGHT, San Diego, CA (US); Gerit Maria POTOTSCHNIG, San Diego, CA (US); Warren Christopher CHAN, San Diego, CA (US); Surendra DAWADI, Longmont, CO (US); Manoj KUMAR, Broomfield, CO (US); Katelyn Frances LONG, Lafayette, CO (US); Alfredo PICADO, Superior, CO (US); Marcos Adrian SAINZ, San Diego, CA (US); Tarek SAMMAKIA, Boulder, CO (US); LaToya Denise SCAGGS, Broomfield, CO (US); Zhicheng SUN, Thornton, CO (US); Erin Danielle ANDERSON, Arvada, CO (US); Shuai CHEN, San Diego, CA (US); Thomas Combs IRVIN, Erie, CO (US); Edward A. KESICKI, New York, NY (US); Xia WANG, San Diego, CA (US); Ganesh Mangalsing MURHADE, Superior, CO (US)

(21) Appl. No.: 18/309,226

(22) Filed: Apr. 28, 2023

Related U.S. Application Data
(60) Provisional application No. 63/496,210, filed on Apr. 14, 2023.
(60) Provisional application No. 63/381,862, filed on Nov. 1, 2022.
(60) Provisional application No. 63/357,689, filed on Jul. 1, 2022.
(60) Provisional application No. 63/336,658, filed on Apr. 29, 2022.

(51) Int. Cl. C07D 311/30; C07D 405/12; C07D 405/14; C07D 409/12; C07D 405/04; C07D 413/12

(52) U.S. Cl. CPC C07D 311/30; C07D 405/12; C07D 413/12; C07D 409/12; C07D 405/04; C07D 405/14

(57) ABSTRACT

The disclosure relates to compounds of Formula (I) as allosteric chromenone inhibitors of phosphoinositide 3-kinase (PI3K) useful in the treatment of diseases or disorders associated with PI3K modulation, Formula (I):

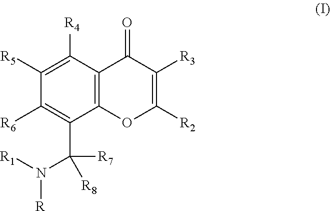

or pharmaceutically acceptable salts thereof wherein R, R₁, R₂, R₃, R₄, R₅, R₆, R₇, and R₈, are as defined herein. The disclosure also relates to methods of making and using compounds of Formula (I) or pharmaceutically acceptable salts thereof.

### CROSS-REFERENCE TO RELATED PATENT APPLICATIONS

The present application claims the benefit of priority under 35 U.S.C. § 119(e) to U.S. Provisional Application No. 63/496,210, filed on Apr. 14, 2023, U.S. Provisional Application No. 63/381,862, filed on Nov. 1, 2022, U.S. Provisional Application No. 63/357,689, filed on Jul. 1, 2022, and U.S. Provisional Application No. 63/336,658, filed on Apr. 29, 2022, the contents of which are incorporated herein by reference in their entireties.

### FIELD

The present invention is directed to allosteric chromenone inhibitors of phosphoinositide 3-kinase (PI3K) useful in the treatment of diseases, or disorders associated with PI3K modulation. The invention is directed toward compounds, and compositions which inhibit PI3K, methods of (or uses for) treating a disease, or disorder associated with PI3K (e.g., CLOVES syndrome (congenital lipomatous overgrowth, vascular malformations, epidermal naevi, scoliosis/skeletal, and spinal syndrome), PIK3CA-related overgrowth syndrome (PROS), breast cancer, brain cancer, prostate cancer, endometrial cancer, gastric cancer, leukemia, lymphoma, sarcoma, colorectal cancer, lung cancer, ovarian cancer, skin cancer, or head and neck cancer), and using, or methods of using, PI3K inhibitors in combination with one or more additional cancer therapies.

### BACKGROUND

The phosphoinositide 3-kinases (PI3Ks) signaling pathway is one of the most highly mutated systems in human cancers. PI3K signaling is involved in many other disease states including allergic contact dermatitis, rheumatoid arthritis, osteoarthritis, inflammatory bowel diseases, chronic obstructive pulmonary disorder, psoriasis, multiple sclerosis, asthma, disorders related to diabetic complications, and inflammatory complications of the cardiovascular system such as acute coronary syndrome.

PI3Ks are members of a unique, and conserved family of intracellular lipid kinases that phosphorylate the 3′-OH group on phosphatidylinositols, or phosphoinositides. The PI3K family comprises 15 kinases with distinct substrate specificities, expression patterns, and modes of regulation (Katso et al., Annu Rev Cell Dev Biol. 2001; 17:615-75). The class I PI3Ks (p110α, p110β, p110δ, and p110γ) are typically activated by tyrosine kinases, or G-protein coupled receptors to generate PIP3, which engages downstream effectors such as those in the pathways of Akt/PDK1, mTOR, the Tec family kinases, and the Rho family GTPases. The class II, and III PI3Ks play a key role in intracellular trafficking through the synthesis of PI(3)P, and PI(3,4)P₂.

The PI3K isoforms have been implicated, for example, in a variety of human cancers, and disorders. Mutations in the gene coding for PI3K isoforms, or mutations which lead to upregulation of a PI3K isoform are believed to occur in many human cancers. Mutations in the gene coding for a PI3K isoform are point mutations clustered within several hotspots in helical, and kinase domains. Because of the high rate of PI3K mutations, targeting of this pathway may provide valuable therapeutic opportunities.

Genetic alterations in genes in PI3K signaling are believed to be involved in a range of cancers such as endometrial cancer, breast cancer, esophageal squamous-cell cancer, cervical squamous-cell carcinoma, cervical adenocarcinoma, colorectal adenocarcinoma, bladder urothelial carcinoma, glioblastoma, ovarian cancer, non-small-cell lung cancer, esophagogastric cancer, nerve-sheath tumor, head and neck squamous-cell carcinoma, melanoma, esophagogastric adenocarcinoma, soft-tissue sarcoma, prostate cancer, fibrolamellar carcinoma, hepatocellular carcinoma, diffuse glioma, colorectal cancer, pancreatic cancer, cholangiocarcinoma, B-cell lymphoma, mesothelioma, adrenocortical carcinoma, renal non-clear-cell carcinoma, renal clear-cell carcinoma, germ-cell carcinoma, thymic tumor, pheochromocytoma, miscellaneous neuroepithelial tumor, thyroid cancer, leukemia, and encapsulated glioma (Goncalves M D, Hopkins B D, Cantley L C. Phosphatidylinositol 3-Kinase, Growth Disorders, and Cancer. N Engl J Med. 2018 Nov. 22; 379(21):2052-2062).

The alpha (a) isoform of PI3K has been implicated, for example, in a variety of human cancers. Angiogenesis has been shown to selectively require the a isoform of PI3K in the control of endothelial cell migration. (Graupera et al, Nature 2008; 453; 662-6). Mutations in the gene coding for PI3Kα, or mutations which lead to upregulation of PI3Kα are believed to occur in many human cancers such as lung, stomach, endometrial, ovarian, bladder, breast, colon, brain, prostate, and skin cancers. Mutations in the gene coding for PI3Kα are point mutations clustered within several hotspots in helical, and kinase domains, such as E542K, E545K, and H1047R. Many of these mutations have been shown to be oncogenic gain-of-function mutations. Because of the high rate of PI3Kα mutations, targeting of this pathway may provide valuable therapeutic opportunities. While other PI3K isoforms such as PI3Kδ, or PI3Kγ are expressed primarily in hematopoietic cells, PI3Kα, along with PI3Kβ, is expressed constitutively.

Mutated PI3Kα has been implicated in brain metastases in HR+/HER2− metastatic breast cancers. Development of brain-penetrant PI3Kα inhibitors may provide improved therapeutic benefit over current PI3Kα inhibitors. (Fitzgerald et al., Association between PIK3CA mutation status and development of brain metastases in HR+/HER2− metastatic breast cancer. Ann Oncol 30:v110, 2019 (suppl 5)).

Due to the central role of PI3Kα in regulating organismal glucose homeostasis, PI3K inhibition in patients often gives rise to hyperglycemia and/or hyperinsulinemia (Busaidy N L, et al, Management of metabolic effects associated with anticancer agents targeting the PI3K-Akt-mTOR pathway. J Clin Oncol 2012; 30:2919-28). High levels of circulating insulin could potentially be mitogenic and/or antiapoptotic for cancer cells, and thus negate the antiproliferative effects of PI3K inhibitors (Blouin M-J, et al, Abstract 4615: the hyperinsulinemia caused by PI3K inhibitors attenuates their antineoplastic efficacy, but can be minimized by co-administration of metformin. Cancer Res 2013; 73:4615).

In the setting of cancer with mutated PI3Kα, one way to overcome the problem of compensatory production of insulin and/or glucose upon systemic PI3Kα inhibition would be to develop inhibitors with enhanced selectivity for mutant PI3Kα over wild-type PI3Kα. This would create an increased window for drug dosing to selectively inhibit the pathologic signaling of mutant PI3Kα in the cancer cells without affecting the wild-type PI3Kα in the host tissues that control systemic metabolism (Okkenhaug K, Graupera M, Vanhaesebroeck B. Targeting PI3K in Cancer: Impact on Tumor Cells, Their Protective Stroma, Angiogenesis, and Immunotherapy. Cancer Discov. 2016 October; 6(10):1090-1105), thus limiting toxicities, and permitting higher doses, and more complete inhibition of the drug target (Ariella B. Hanker, et al, Challenges for the clinical development of PI3K inhibitors: Strategies to improve their impact in solid tumors. Cancer Discov. 2019 April; 9(4): 482-491).

Currently PI3Kα inhibitors are nearly equipotent to wild-type, and mutant PI3Kα. Mutant selective inhibitors have been elusive due to the PI3Kα mutations location far from the active site. As such, inhibitors which target a second, peripheral binding pocket near a known mutation (e.g., H1047R) may provide a route to selective PI3Kα inhibition. Thus, targeting a mutated, peripheral binding pocket of PI3Kα, provides a valuable therapeutic target for drug development.

As such, kinases, for example lipid kinases such as PI3Ks, are prime targets for drug development. The present invention provides new kinase inhibitors.

### SUMMARY

In one aspect, the present invention relates to compounds of Formula (I):

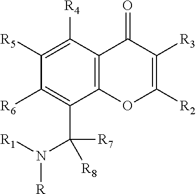

or pharmaceutically acceptable salt thereof, wherein:
R and R₁ together with the nitrogen to which they are attached form an optionally substituted bicyclic ring selected from dihydrobenzimidazolone, dihydroindazolone, indolinone or quinazolinone; wherein the optionally substituted bicyclic ring is optionally substituted with one to three substituents each independently selected from oxo, —CN, halogen, C₁-C₆ alkyl, C₁-C₆ haloalkyl, —OH or C₁-C₆ alkoxy; or
R is —H or C₁-C₃ alkyl; R₁ is an optionally substituted bicyclic ring selected from isobenzofuranone,
benzofuranone, indole, isoindolinone, indolinone, quinazolinone, 3,4-dihydro-2H-isoquinolin-1-one, 2H-isoquinolin-1-one, imidaza[1,2-a]pyridine, or benzothiazolone; wherein the optionally substituted bicyclic ring is optionally substituted with one to three substituents each independently selected from oxo, —CN, halogen, C₁-C₆ alkyl, C₁-C₆ haloalkyl, —OH or C₁-C₆ alkoxy; or
R₁ is a group of the formula:

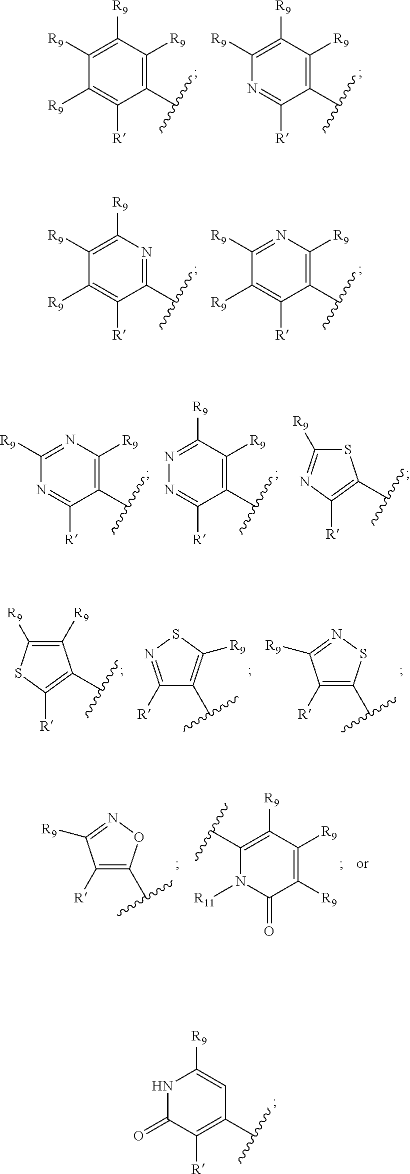

R′ is hydrogen, halogen, C₁-C₃ alkyl, C₁-C₃ haloalkyl, C₁-C₃ hydroxyalkyl, C₁-C₃ alkoxy, C₁-C₃ haloalkoxy, —OH, —CH(OH)—CH₂OH, —CH(OH)C₁-C₃ haloalkyl, —C(O)—CH₂OH, C₃-C₆ cycloalkyl, —NO₂, —NR₁₁R₁₁, —N(R₁₁)—CO₂C₁-C₃ alkyl, —N(R₁₁)—SO₂C₁-C₃ alkyl, —N(R₁₁)—SO₂R₁₅, —SO₂C₁-C₃ alkyl, —S(O)C₁-C₃ alkyl, —SO₂NR₁₁R₁₁, —SO₂N(R₁₁)—C(O)—C₁-C₃ alkyl, —SO₂N(R₁₁)—CN, —SO₂N(R₁₁)(R₁₃)—C(═N—OH)—NH₂, —CN, —CO₂C₁-C₃ alkyl, —C(O)NR₁₁R₁₂, —C(O)N(R₁₁)—(CH₂)ₙ—R₁₃, —C(O)—SR₁₂, —C(O)—NHSO₂R₁₆, —C(O)CH═SOR₁₁(R₁₁), or —C(O)CH₂CN, or a group of the formula:

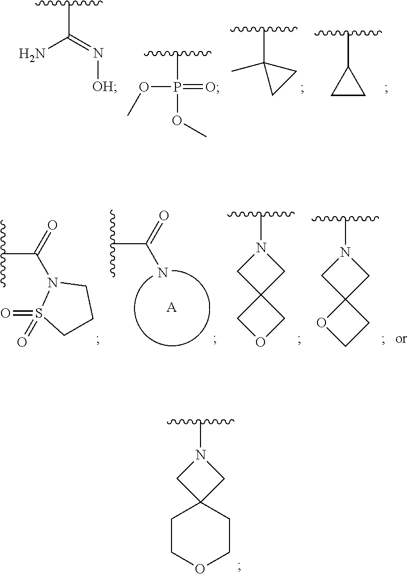

wherein ring A is pyrrolidine optionally substituted with a —CN; or
R′ is selected from oxetane, azetidine, pyrrolidine, tetrahydrofuran, morpholine, thiomorpholine, piperidine, piperazine, pyrrole, furan, thiophene, pyrazole, imidazole, isoxazole, oxazole, isothiazole, thiazole, triazole, oxadiazole, 1,2,4-oxadiazolin-5-one, 1,4-oxazepane, thiadiazole, tetrazole, phenyl, pyridine, pyridazine, pyrimidine, pyrazine, triazine, or oxazepane; each of which is optionally substituted with one to three substituents independently selected from oxo, —OH, —NR₁₁R₁₁, —N(R₁₁)—C(O)—R₁₁, —N(R₁₁)—CN, —OH, C₁-C₃ alkoxy, —CN, halogen, morpholino, oxetane, C₁-C₆ haloalkyl or C₁-C₆ alkyl optionally substituted with —OH, an aryl, a 5-member heteroaryl, or a 6-member heteroaryl; or
R′ is a group of the formula:

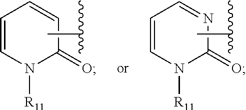

R₂ is a group of the formula:

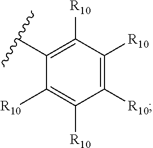

R₂ is a group of the formula:

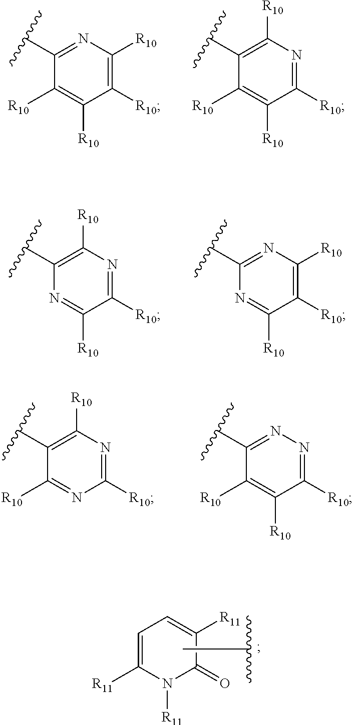

or
R₂ is an optionally substituted 5-member ring heteroaryl selected from pyrrole, furan, thiophene, pyrazole, isoxazole, isothiazole, imidazole, oxazole, thiazole, triazole, tetrazole, oxadiazole, and thiadiazole; wherein the optionally substituted 5-member ring heteroaryl is optionally substituted with one to three substituents each independently selected from —CN, halogen, C₁-C₆ haloalkyl, C₁-C₆ alkoxy, C₁-C₆ haloalkoxy, —SO₂R₁₁, —CO₂C₁-C₃ alkyl, —C(O)NR₁₁R₁₁, —OH, —NR₁₁R₁₁, —NR₁₁CO₂R₁₁, —CD₃, an optionally substituted C₁-C₆ alkyl, an optionally substituted C₂-C₆ alkenyl, an optionally substituted C₂-C₆ alkynyl, an optionally substituted C₃-C₅ cycloalkyl, an optionally substituted heterocycle selected from pyrrolidine, pyrrolidinone, piperidine, tetrahydropyran, oxetane, azetidine, or morpholine, an optionally substituted phenyl, an optionally substituted 1,3-benzodioxole, an optionally substituted 2,3-dihydro-1,4-benzodioxine, or an optionally substituted heteroaryl selected from pyridine, pyrimidine, pyridazine, pyrazine, pyrazole, isoxazole, isothiazole, imidazole, oxazole, or thiazole; wherein the optionally substituted C₁-C₆ alkyl, C₂-C₆ alkenyl, or C₂-C₆ alkynyl is each optionally substituted with a —CN, —OH, oxetanyl, C₁-C₃ alkoxy, —CH₂CH(OH)CH(OH)R₁₁; or —C(O)NR₁₁R₁₁; the optionally substituted C₃-C₅ cycloalkyl, phenyl, 1,3-benzodioxole, 2,3-dihydro-1,4-benzodioxine, heterocycle or heteroaryl is each optionally substituted with one to three substituents each independently selected from halogen, C₁-C₃ alkyl, C₁-C₃ haloalkyl, C₁-C₃ alkoxy, C₁-C₃ haloalkoxy, —C(O(CH₂)ₙOR₁₁, —C(O)C(CH₃)₂OH, —SO₂R₁₁, —NR₁₁R₁₁, —OH or —CN; or
R₂ is an optionally substituted fused bicyclic ring selected from 1,3-benzodioxole, 2,3-dihydro-1,4-benzodioxine, indole, indazole, isoindazole, isoindolin-1-one, indolin-2-one, benzoxazole, benzotriazole, benzo[d]oxazol-2(3H)-one, 1,3-dihydro-2H-pyrrolo[2,3-b]pyridin-2-one, 6,7-dihydro-4H-pyrazolo[5,1-c][1,4]oxazine, 4,5,6,7-tetrahydropyrazolo[1,5-a]pyrazine, or 2,3-dihydro-[1,4]dioxino[2,3-b]pyridine, imidazo[1,2-a]pyridine, pyrazolo[4,3-b]pyridine, pyrazolo[3,4-b]pyridine, pyrazolo[3,4-c]pyridine, pyrazolo[1,5-a]pyrimidine, or an optionally substituted bicyclic heteroaryl of 8 to 10 ring atoms containing 1, 2, 3, 4, or 5 ring heteroatoms independently selected from N, O, or S; wherein the optionally substituted bicyclic ring is optionally substituted with one to three substituents each independently selected from halogen and C₁-C₆ alkyl; the optionally substituted bicyclic heteroaryl is optionally substituted with one to three substituents each independently selected from —CN, halogen, C₁-C₆ haloalkyl, C₁-C₆ alkoxy, C₁-C₆ haloalkoxy, —SO₂R₁₁, —CO₂H, —CO₂C₁-C₃ alkyl, —C(O)NR₁₁R₁₁, —NR₁₁R₁₁, —NR₁₁CO₂R₁₁, —OH, an optionally substituted C₁-C₆ alkyl, an optionally substituted C₂-C₆ alkenyl, an optionally substituted C₂-C₆ alkynyl, an optionally substituted C₃-C₅ cycloalkyl, an optionally substituted heterocycle selected from pyrrolidine, pyrrolidinone, piperidine or morpholine, an optionally substituted phenyl, an optionally substituted 1,3-benzodioxole, an optionally substituted 2,3-dihydro-1,4-benzodioxine, or an optionally substituted heteroaryl selected from pyridine, pyrimidine, pyridazine, pyrazine, pyrazole, isoxazole, isothiazole, imidazole, oxazole, or thiazole; wherein the optionally substituted C₁-C₆ alkyl, C₂-C₆ alkenyl, or C₂-C₆ alkynyl is each optionally substituted with a —CN, —OH, oxetanyl, C₁-C₃ alkoxy, —C(O)NR₁₁R₁₁ or phenyl; the optionally substituted C₃-C₅ cycloalkyl, phenyl, 1,3-benzodioxole, 2,3-dihydro-1,4-benzodioxine, heterocycle or heteroaryl is each optionally substituted with one to three substituents each independently selected from halogen, C₁-C₃ alkyl, C₁-C₃ haloalkyl, C₁-C₃ alkoxy, C₁-C₃ haloalkoxy, —SO₂R₁₁, —NR₁₁R₁₁, —OH or —CN; or
R₂ is a cyclopropyl, cyclobutyl, cyclopentyl, bicyclo[1.1.1]pentane, bicyclo[2.2.2]octane, or cyclohexyl, each optionally substituted with one to three R₁₀ substituents, or R₂ is a group of the formula:

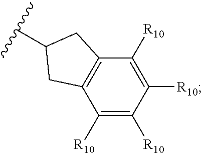

R₃ is —H, halogen, —CN, —C(CN)═CHOH, —N(H)(C₁-C₃ alkyl), —N(C₁-C₃ alkyl)₂, —N(H)(CH₂CH₂CO₂H), —C(O)—C₁-C₃ alkyl, C₁-C₆ alkyl, C₁-C₆ haloalkyl, C₁-C₆ hydroxyalkyl, C₃-C₅ cycloalkyl, an optionally substituted heterocycle of 3 to 5 ring atoms containing 1, 2, or 3 ring heteroatoms independently selected from N, O, or S, or an optionally substituted heteroaryl of 5 or 6 ring atoms containing 1, 2, or 3 ring heteroatoms independently selected from N, O, or S; wherein the optionally substituted heterocycle or heteroaryl is each optionally substituted with one to three substituents each independently selected from halogen, C₁-C₃ alkyl, or C₁-C₃ haloalkyl;
each of R₄, R₅ and R₆ is independently —H, halogen, —CN, C₁-C₆ alkyl or C₁-C₆ haloalkyl;
R₇ is —CN, C₁-C₆ alkyl, —CH₂OH, or C₁-C₆ haloalkyl;
R₈ is —H, -D, or C₁-C₆ alkyl;
each R₉ is independently —H, halogen, —CN, C₁-C₆ alkyl, C₁-C₆ haloalkyl, C₁-C₆ alkoxy, or C₃-C₅ cycloalkyl;
each R₁₀ is independently —H, —CN, halogen, C₁-C₆ haloalkyl, C₁-C₆ alkoxy, C₁-C₆ haloalkoxy, —SO₂R₁₁, —SONR₁₁R₁₁, —CO₂H, —CO₂C₁-C₃ alkyl, —C(O)NR₁₁R₁₂, —NR₁₁R₁₁, —NR₁₁—CO₂R₁₁, —N(R₁₁)C(O)R₁₁, —OH, an optionally substituted C₁-C₆ alkyl, an optionally substituted C₂-C₆ alkenyl, an optionally substituted C₂-C₆ alkynyl, an optionally substituted C₃-C₅ cycloalkyl, an optionally substituted heterocycle selected from pyrrolidine, pyrrolidinone, piperidine or morpholine, an optionally substituted phenyl, an optionally substituted 1,3-benzodioxole, an optionally substituted 2,3-dihydro-1,4-benzodioxine, or an optionally substituted heteroaryl selected from pyrazole, isoxazole, isothiazole, imidazole, oxazole, thiazole, or pyridine, or a group of the formula:

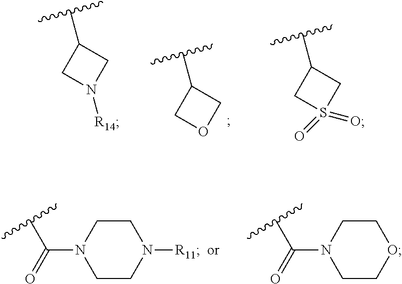

wherein the optionally substituted C₁-C₆ alkyl, C₂-C₆ alkenyl, or C₂-C₆ alkynyl is each optionally substituted with a —CN, —OH, oxetanyl, C₁-C₃ alkoxy, or —C(O)NR₁₁R₁₁; the optionally substituted C₃-C₅ cycloalkyl, phenyl, 1,3-benzodioxole, 2,3-dihydro-1,4-benzodioxine, heterocycle or heteroaryl is each optionally substituted with one to three substituents each independently selected from halogen, C₁-C₃ alkyl, C₁-C₃ haloalkyl, C₁-C₃ alkoxy, C₁-C₃ haloalkoxy, —SO₂R₁₁, —NR₁₁R₁₁, —OH or —CN;
each R₁₁ is independently —H, C₁-C₃ alkyl, C₃-C₇ cycloalkyl, or C₁-C₃ haloalkyl;
each R₁₂ is independently —H, optionally substituted C₁-C₃ alkyl, C₃-C₆ alkoxy, C₃-C₆ cycloalkyl, —SO₂C₁-C₃ alkyl, —SO₂C₁-C₃ haloalkyl, —SO₂NR₁₁R₁₁, —NR₁₁R₁₁, —OR₁₁, —O—CH₂—CH(OH)—CH₂OH, —CN, oxetane, tetrahydrofuran, an aryl, a 5-member heteroaryl optionally substituted with a methyl, a 6-member heteroaryl, or a group of the formula

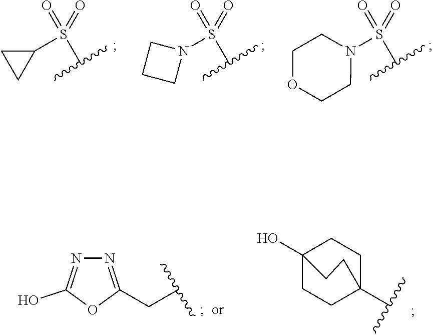 [SMILES: *S(=O)(=O)C1CC1 ; *S(=O)(=O)N1CCC1 ; *S(=O)(=O)N1CCOCC1 ; *Cc1nnc(O)o1 ; *C12CCC(O)(CC1)CC2]

wherein the optionally substituted C₁-C₃ alkyl is optionally substituted with —OH, a C₃-C₆ cycloalkyl, oxetane, tetrahydrofuran, an aryl, a 5-member heteroaryl, a 6-member heteroaryl or an indole;
R₁₃ is —NR₁₁R₁₁, —OR₁₁, —SO₂C₁-C₃ alkyl, or a ring selected from oxetane, tetrahydrofuran or oxadiazole wherein the ring is optionally substituted with —NR₁₁R₁₁, or —OR₁₁;
R₁₄ is —H, optionally substituted C₁-C₃ alkyl, —SO₂C₁-C₃ alkyl, an aryl, a 5-member heteroaryl, or a 6-member heteroaryl; wherein the optionally substituted C₁-C₃ alkyl is optionally substituted with an aryl, a 5-member heteroaryl or a 6-member heteroaryl;
R₁₅ is an optionally substituted aryl, or an optionally substituted 6-member heteroaryl; wherein the optionally substituted aryl, or the optionally substituted 6-member heteroaryl is each optionally substituted with one to three substituents each independently selected from halogen, C₁-C₃ alkyl, or C₁-C₃ haloalkyl;
R₁₆ is H, C₁-C₃ alkyl, —NH₂, phenyl, or pyridine, and
n is 0, 1 or 2.

In one aspect, the compounds of Formula (I) may include an asymmetrical carbon atom at the position designated with an asterisk (*)

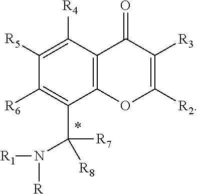

In one aspect, R₇ is methyl and R₈ is hydrogen and the bond at the * position is as represented as

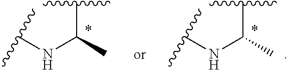

In one aspect, the present invention relates to compounds of Formula (I):

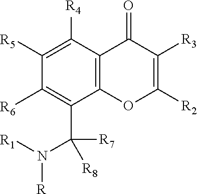

or pharmaceutically acceptable salt thereof, wherein:
R and R₁ together with the nitrogen to which they are attached form an optionally substituted bicyclic ring selected from dihydrobenzimidazolone, dihydroindazolone, indolinone or quinazolinone; wherein the optionally substituted bicyclic ring is optionally substituted with one to three substituents each independently selected from oxo, —CN, halogen, C₁-C₆ alkyl, C₁-C₆ haloalkyl, —OH or C₁-C₆ alkoxy; or
R is —H or C₁-C₃ alkyl;
R₁ is an optionally substituted bicyclic ring selected from isobenzofuranone, benzofuranone, isoindolinone, indolinone, quinazolinone, 3,4-dihydro-2H-isoquinolin-1-one, 2H-isoquinolin-1-one, or benzothiazolone; wherein the optionally substituted bicyclic ring is optionally substituted with one to three substituents each independently selected from oxo, —CN, halogen, C₁-C₆ alkyl, C₁-C₆ haloalkyl, —OH or C₁-C₆ alkoxy; or
R₁ is a group of the formula:

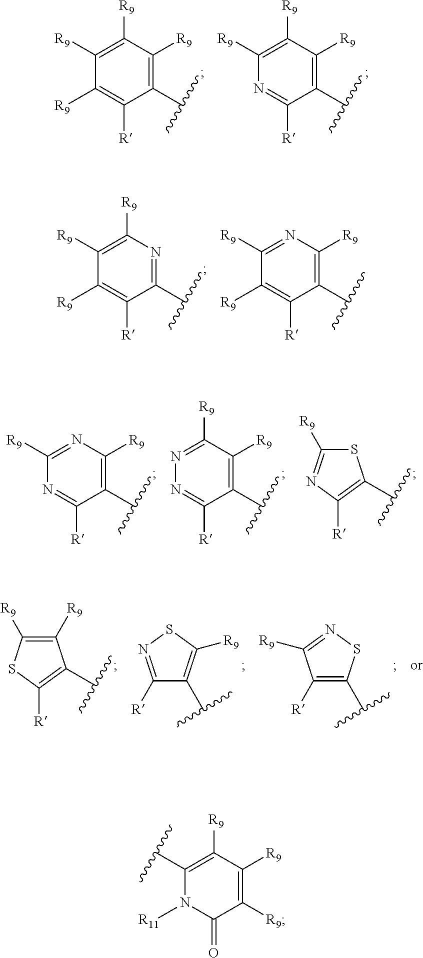

R′ is hydrogen, halogen, C₁-C₃ alkyl, C₁-C₃ haloalkyl, C₁-C₃ alkoxy, C₁-C₃ haloalkoxy, —OH, —CH(OH)—CH₂OH, —CO—CH₂OH, C₃-C₆ cycloalkyl, —NO₂, —N(R₁₁)—CO₂C₁-C₃ alkyl, —N(R₁₁)—SO₂C₁-C₃ alkyl, —N(R₁₁)—SO₂R₁₅, —SO₂C₁-C₃ alkyl, —SO₂NR₁₁R₁₁, —SO₂N(R₁₁)—CO—C₁-C₃ alkyl, —SO₂N(R₁₁)—CN, —C(═N—OH)—NH₂, —CN, —CO₂C₁-C₃ alkyl, —CONR₁₁R₁₂, —CON(R)—(CH₂)ₙ—R₁₃, —CO—SR₁₂, or a group of the formula:

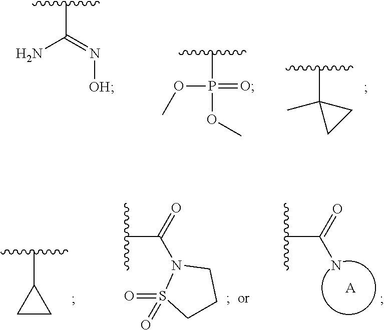

wherein ring A is pyrrolidine optionally substituted with a —CN; or
R′ is selected from oxetane, pyrrolidine, tetrahydrofuran, morpholine, piperidine, piperazine, pyrrole, furan, thiophene, pyrazole, imidazole, isoxazole, oxazole, isothiazole, thiazole, triazole, oxadiazole, thiadiazole, tetrazole, phenyl, pyridine, pyridazine, pyrimidine, pyrazine, or triazine; each of which is optionally substituted with one to three substituents independently selected from oxo, —OH, —NR₁₁R₁₁, —N(R₁₁)—CO—R₁₁, —N(R₁₁)—CN, —OR₁₁, —CN, halogen, C₁-C₆ haloalkyl or C₁-C₆ alkyl optionally substituted with an aryl, a 5-member heteroaryl or a 6-member heteroaryl; or
R′ is a group of the formula:

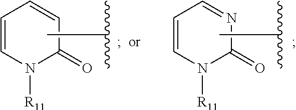

R₂ is a group of the formula:

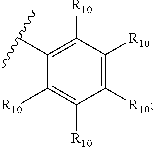

or
R₂ is a group of the formula:

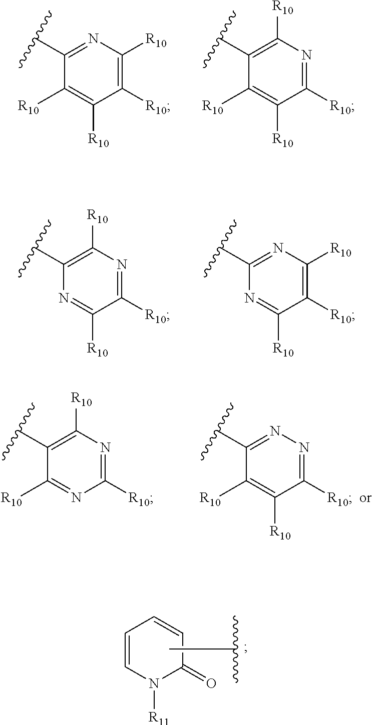

or
R₂ is an optionally substituted 5-member ring heteroaryl selected from pyrrole, furan, thiophene, pyrazole, isoxazole, isothiazole, imidazole, oxazole, thiazole, triazole, tetrazole, oxadiazole, and thiadiazole; wherein the optionally substituted 5-member ring heteroaryl is optionally substituted with one to three substituents each independently selected from —CN, halogen, C₁-C₆ haloalkyl, C₁-C₆ alkoxy, C₁-C₆ haloalkoxy, —SO₂R₁₁, —CO₂C₁-C₃ alkyl, —CONR₁₁R₁₁, —OH, —NR₁₁R₁₁, —NR₁₁CO₂R₁₁, an optionally substituted C₁-C₆ alkyl, an optionally substituted C₂-C₆ alkenyl, an optionally substituted C₂-C₆ alkynyl, an optionally substituted C₃-C₅ cycloalkyl, an optionally substituted heterocycle selected from pyrrolidine, pyrrolidinone, piperidine or morpholine, an optionally substituted phenyl, an optionally substituted 1,3-benzodioxole, an optionally substituted 2,3-dihydro-1,4-benzodioxine, or an optionally substituted heteroaryl selected from pyridine, pyrimidine, pyridazine, pyrazine, pyrazole, isoxazole, isothiazole, imidazole, oxazole, or thiazole; wherein the optionally substituted C₁-C₆ alkyl, C₂-C₆ alkenyl, or C₂-C₆ alkynyl is each optionally substituted with a —CN, —OH, oxetanyl, C₁-C₃ alkoxy, or —CONR₁₁R₁₁; the optionally substituted C₃-C₅ cycloalkyl, phenyl, 1,3-benzodioxole, 2,3-dihydro-1,4-benzodioxine, heterocycle or heteroaryl is each optionally substituted with one to three substituents each independently selected from halogen, C₁-C₃ alkyl, C₁-C₃ haloalkyl, C₁-C₃ alkoxy, C₁-C₃ haloalkoxy, —SO₂R₁₁, —NR₁₁R₁₁, —OH or —CN; or
R₂ is an optionally substituted bicyclic ring selected from 1,3-benzodioxole, 2,3-dihydro-1,4-benzodioxine, isoindolin-1-one, indolin-2-one, benzo[d]oxazol-2(3H)-one, 1,3-dihydro-2H-pyrrolo[2,3-b]pyridin-2-one, or 2,3-dihydro-[1,4]dioxino[2,3-b]pyridine, or an optionally substituted bicyclic heteroaryl of 8 to 10 ring atoms containing 1, 2, 3, 4, or 5 ring heteroatoms independently selected from N, O, or S; wherein the optionally substituted bicyclic ring is optionally substituted with one to three substituents each independently selected from halogen and C₁-C₆ alkyl; the optionally substituted bicyclic heteroaryl is optionally substituted with one to three substituents each independently selected from —CN, halogen, C₁-C₆ haloalkyl, C₁-C₆ alkoxy, C₁-C₆ haloalkoxy, —SO₂R₁₁, —CO₂C₁-C₃ alkyl, —CONR₁₁R₁₁, —NR₁₁R₁₁, —NR₁₁CO₂R₁₁, —OH, an optionally substituted C₁-C₆ alkyl, an optionally substituted C₂-C₆ alkenyl, an optionally substituted C₂-C₆ alkynyl, an optionally substituted C₃-C₅ cycloalkyl, an optionally substituted heterocycle selected from pyrrolidine, pyrrolidinone, piperidine or morpholine, an optionally substituted phenyl, an optionally substituted 1,3-benzodioxole, an optionally substituted 2,3-dihydro-1,4-benzodioxine, or an optionally substituted heteroaryl selected from pyridine, pyrimidine, pyridazine, pyrazine, pyrazole, isoxazole, isothiazole, imidazole, oxazole, or thiazole; wherein the optionally substituted C₁-C₆ alkyl, C₂-C₆ alkenyl, or C₂-C₆ alkynyl is each optionally substituted with a —CN, —OH, oxetanyl, C₁-C₃ alkoxy, —CONR₁₁R₁₁ or phenyl; the optionally substituted C₃-C₅ cycloalkyl, phenyl, 1,3-benzodioxole, 2,3-dihydro-1,4-benzodioxine, heterocycle or heteroaryl is each optionally substituted with one to three substituents each independently selected from halogen, C₁-C₃ alkyl, C₁-C₃ haloalkyl, C₁-C₃ alkoxy, C₁-C₃ haloalkoxy, —SO₂R₁₁, —NR₁₁R₁₁, —OH or —CN; or
R₂ is a cyclopropyl, cyclobutyl, cyclopentyl, or cyclohexyl, each optionally substituted with one to three R₁₀ substituents, or R₂ is a group of the formula:

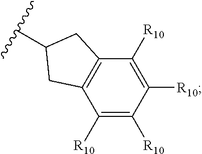

R₃ is —H, halogen, —CN, —N(H)(C₁-C₃ alkyl), —N(C₁-C₃ alkyl)₂, —N(H)(CH₂CH₂CO₂H), —CO—C₁-C₃ alkyl, C₁-C₆ alkyl, C₁-C₆ haloalkyl, C₁-C₆ hydroxyalkyl, C₃-C₅ cycloalkyl, an optionally substituted heterocycle of 3 to 5 ring atoms containing 1, 2, or 3 ring heteroatoms independently selected from N, O, or S, or an optionally substituted heteroaryl of 5 or 6 ring atoms containing 1, 2, or 3 ring heteroatoms independently selected from N, O, or S; wherein the optionally substituted heterocycle or heteroaryl is each optionally substituted with one to three substituents each independently selected from halogen, C₁-C₃ alkyl, or C₁-C₃ haloalkyl; each of R₄, R₅ and R₆ is independently —H, halogen, C₁-C₆ alkyl or C₁-C₆ haloalkyl;
R₇ is —CN, C₁-C₆ alkyl or C₁-C₆ haloalkyl;
R₈ is —H or C₁-C₆ alkyl;
each R₉ is independently —H, halogen, —CN, C₁-C₆ alkyl, C₁-C₆ haloalkyl, C₁-C₆ alkoxy, or C₃-C₅ cycloalkyl;
each R₁₀ is independently —H, —CN, halogen, C₁-C₆ haloalkyl, C₁-C₆ alkoxy, C₁-C₆ haloalkoxy, —SO₂R₁₁, —SONR₁₁R₁₁, —CO₂H, —CO₂C₁-C₃ alkyl, —CONR₁₁R₁₂, —NR₁₁R₁₁, —NR₁₁—CO₂R₁₁, —OH, an optionally substituted C₁-C₆ alkyl, an optionally substituted C₂-C₆ alkenyl, an optionally substituted C₂-C₆ alkynyl, an optionally substituted C₃-C₅ cycloalkyl, an optionally substituted heterocycle selected from pyrrolidine, pyrrolidinone, piperidine or morpholine, an optionally substituted phenyl, an optionally substituted 1,3-benzodioxole, an optionally substituted 2,3-dihydro-1,4-benzodioxine, or an optionally substituted heteroaryl selected from pyrazole, isoxazole, isothiazole, imidazole, oxazole, thiazole or pyridine, or a group of the formula:

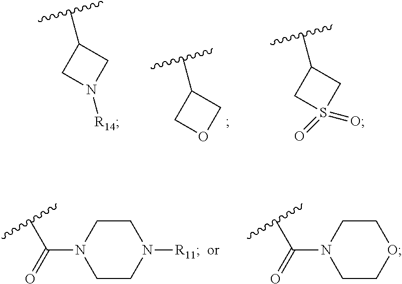

wherein the optionally substituted C₁-C₆ alkyl, C₂-C₆ alkenyl, or C₂-C₆ alkynyl is each optionally substituted with a —CN, —OH, oxetanyl, C₁-C₃ alkoxy, or —CONR₁₁R₁₁; the optionally substituted C₃-C₅ cycloalkyl, phenyl, 1,3-benzodioxole, 2,3-dihydro-1,4-benzodioxine, heterocycle or heteroaryl is each optionally substituted with one to three substituents each independently selected from halogen, C₁-C₃ alkyl, C₁-C₃ haloalkyl, C₁-C₃ alkoxy, C₁-C₃ haloalkoxy, —SO₂R₁₁, —NR₁₁R₁₁, —OH or —CN;
each R₁₁ is independently —H or C₁-C₃ alkyl;
each R₁₂ is independently —H, optionally substituted C₁-C₃ alkyl, C₃-C₆ cycloalkyl, —SO₂C₁-C₃ alkyl, —SO₂C₁-C₃ haloalkyl, —SO₂NR₁₁R₁₁, —NR₁₁R₁₁, —OR₁₁, —O—CH₂—CH(OH)—CH₂OH, —CN, oxetane, tetrahydrofuran, an aryl, a 5-member heteroaryl optionally substituted with a methyl, a 6-member heteroaryl, or a group of the formula

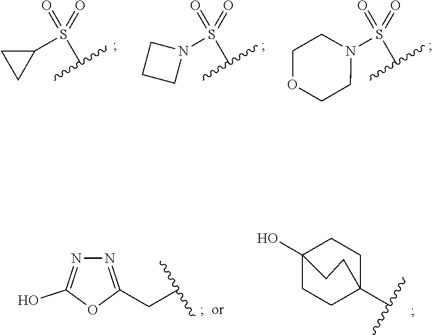 [SMILES: *S(=O)(=O)C1CC1 ; *S(=O)(=O)N1CCC1 ; *S(=O)(=O)N1CCOCC1 ; *Cc1nnc(O)o1 ; *C12CCC(O)(CC1)CC2]

wherein the optionally substituted C₁-C₃ alkyl is optionally substituted with —OH, a C₃-C₆ cycloalkyl, oxetane, tetrahydrofuran, an aryl, a 5-member heteroaryl, a 6-member heteroaryl or an indole;
R₁₃ is —NR₁₁R₁₁, —OR₁₁, —SO₂C₁-C₃ alkyl, or a ring selected from oxetane, tetrahydrofuran or oxadiazole wherein the ring is optionally substituted with —NR₁₁R₁₁, or —OR₁₁;
R₁₄ is —H, optionally substituted C₁-C₃ alkyl, —SO₂C₁-C₃ alkyl, an aryl, a 5-member heteroaryl, or a 6-member heteroaryl; wherein the optionally substituted C₁-C₃ alkyl is optionally substituted with an aryl, a 5-member heteroaryl or a 6-member heteroaryl;
R₁₅ is an optionally substituted aryl, or an optionally substituted 6-member heteroaryl; wherein the optionally substituted aryl, or the optionally substituted 6-member heteroaryl is each optionally substituted with one to three substituents each independently selected from halogen, C₁-C₃ alkyl, or C₁-C₃ haloalkyl;
and
n is 0, 1 or 2.

In another aspect, the present invention provides a pharmaceutical composition comprising a compound of Formula (I), (II), or (III), or a pharmaceutically acceptable salt thereof, and a pharmaceutically acceptable diluent, or carrier.

In another aspect, the present invention provides a method of modulating PI3K (e.g., PI3Kα) activity (e.g., in vitro, or in vivo), comprising contacting a cell with a therapeutically effective amount of a compound of Formula (I), (II), or (III), or a pharmaceutically acceptable salt thereof.

In some aspects, the present invention provides a method of treating, or preventing a disease, or disorder disclosed herein in a subject in need thereof, comprising administering to the subject a therapeutically effective amount of a compound of Formula (I), (II), or (III), or a pharmaceutically acceptable salt thereof.

In some aspects, the present invention provides a method of treating, or preventing a disease, or disorder disclosed herein in a subject in need thereof, comprising administering to the subject a therapeutically effective amount of a pharmaceutical composition of a compound of Formula (I), (II), or (III), or a pharmaceutically acceptable salt thereof.

In some aspects, the present invention provides a method of treating a disease, or disorder disclosed herein in a subject in need thereof, comprising administering to the subject a therapeutically effective amount of a compound of Formula (I), (II), or (III), or a pharmaceutically acceptable salt thereof.

In some aspects, the present invention provides a method of treating a disease, or disorder disclosed herein in a subject in need thereof, comprising administering to the subject a therapeutically effective amount of a pharmaceutical composition of a compound of Formula (I), (II), or (III), or a pharmaceutically acceptable salt thereof.

In another aspect, the present invention provides a compound of Formula (I), (II), or (III), or a pharmaceutically acceptable salt thereof for use in therapy.

In another aspect, the present invention provides a compound of Formula (I), (II), or (III), or a pharmaceutically acceptable salt thereof for use in modulating PI3K (e.g., PI3Kα) activity (e.g., in vitro, or in vivo).

In another aspect, the present invention provides a compound of Formula (I), (II), or (III), or a pharmaceutically acceptable salt thereof, for use in selective inhibition for mutant PI3Kα over wild-type PI3Kα.

In another aspect, the present invention provides a compound of Formula (I), (II), or (III), or a pharmaceutically acceptable salt thereof, for use in treating, or preventing a disease, or disorder disclosed herein.

In another aspect, the present invention provides a compound of Formula (I), (II), or (III), or a pharmaceutically acceptable salt thereof, for use in treating a disease, or disorder disclosed herein.

In another aspect, the present invention provides use of a compound of Formula (I), (II), or (III), or a pharmaceutically acceptable salt thereof, in the manufacture of a medicament for modulating PI3K (e.g., PI3Kα) activity (e.g., in vitro, or in vivo).

In another aspect, the present invention provides use of a compound of Formula (I), (II), or (III), or a pharmaceutically acceptable salt thereof, in the manufacture of a medicament for treating, or preventing a disease, or disorder disclosed herein.

In another aspect, the present invention provides use of a compound of Formula (I), (II), or (III), or a pharmaceutically acceptable salt thereof, in the manufacture of a medicament for treating a disease, or disorder disclosed herein.

In another aspect, the present invention provides a method of preparing a compound of Formula (I), (II), or (III), or a pharmaceutically acceptable salt thereof.

In another aspect, the present invention provides a method of preparing a compound, of Formula (I), (II), or (III), or a pharmaceutically acceptable salt thereof, comprising one, or more steps described herein.

In another aspect, the present invention provides a compound obtainable by, or obtained by, a method for preparing a compound as described herein.

In another aspect, the present invention provides an intermediate as described herein, being suitable for use in a method for preparing a compound as described herein (e.g., the intermediate is selected from the intermediates described in the Examples).

Other features, and advantages of the invention will be apparent from the following detailed description, and claims.

### DETAILED DESCRIPTION

The present invention provides methods of treating, preventing, or ameliorating a disease, or disorder, (or uses in the treatment, prevention, or amelioration of a disease, or disorder), in which PI3K plays a role by administering to a patient in need thereof a therapeutically effective amount of a PI3K inhibitor of the present invention. The methods (or uses) of the present invention can be used in the treatment of a variety of PI3K-dependent diseases, and disorders.

In some embodiments, the disease, or disorder is a cancer (e.g., breast cancer, brain cancers, prostate cancer, endometrial cancer, gastric cancer, leukemia, lymphoma, sarcoma, colorectal cancer, lung cancer, ovarian cancer, skin cancer, or head and neck cancer). In some embodiments, the disease, or disorder associated with PI3K includes, but is not limited to, CLOVES syndrome, PROS, endometrial cancer, breast cancer, esophageal squamous-cell cancer, cervical squamous-cell carcinoma, cervical adenocarcinoma, colorectal adenocarcinoma, bladder urothelial carcinoma, glioblastoma, ovarian cancer, non-small-cell lung cancer, esophagogastric cancer, nerve-sheath tumor, head and neck squamous-cell carcinoma, melanoma, esophagogastric adenocarcinoma, soft-tissue sarcoma, prostate cancer, fibrolamellar carcinoma, hepatocellular carcinoma, diffuse glioma, colorectal cancer, pancreatic cancer, cholangiocarcinoma, B-cell lymphoma, mesothelioma, adrenocortical carcinoma, renal non-clear-cell carcinoma, renal clear-cell carcinoma, germ-cell carcinoma, thymic tumor, pheochromocytoma, miscellaneous neuroepithelial tumor, thyroid cancer, leukemia, and encapsulated glioma.

The details of the invention are set forth in the accompanying description below. Although methods, and materials similar, or equivalent to those described herein can be used in the practice, or testing of the present disclosure, illustrative methods, and materials are now described. Other features, objects, and advantages of the invention will be apparent from the description, and from the claims. In the specification, and the appended claims, the singular forms also include the plural unless the context clearly dictates otherwise. Unless defined otherwise, all technical, and scientific terms used herein have the same meaning as commonly understood by one of ordinary skill in the art to which this invention belongs. All patents, and publications cited in this specification are incorporated herein by reference in their entireties.

### Definitions

The articles “a”, and “an” refer to one, or more than one (i.e., to at least one) of the grammatical object of the article. By way of example, “an element” means one element, or more than one element.

The term “and/or” means either “and”, or “or” unless indicated otherwise.

The term “administer”, “administering”, or “administration” refers to either directly administering a disclosed compound, or pharmaceutically acceptable salt of the disclosed compound, or a composition to a subject.

The term “alkenyl” refers to a straight, or branched chain unsaturated hydrocarbon containing 2-12 carbon atoms. The “alkenyl” group contains at least one double bond in the chain. The double bond of an alkenyl group can be unconjugated, or conjugated to another unsaturated group. Examples of alkenyl groups include ethenyl, propenyl, n-butenyl, iso-butenyl, pentenyl, or hexenyl.

The term “alkoxy” refers to a straight, or branched chain saturated hydrocarbon containing 1-12 carbon atoms containing a terminal “O” in the chain, i.e., —O(alkyl). Examples of alkoxy groups include without limitation, methoxy, ethoxy, propoxy, butoxy, t-butoxy, or pentoxy groups.

The term “alkyl” refers to a straight, or branched chain saturated hydrocarbon containing 1-12 carbon atoms, preferably 1-6 carbon atoms. Examples of a C₁-C₆ alkyl group include, but are not limited to, methyl, ethyl, propyl, butyl, pentyl, hexyl, isopropyl, isobutyl, sec-butyl, tert-butyl, isopentyl, neopentyl, and isohexyl.

The term “alkynyl” refers to a straight, or branched chain unsaturated hydrocarbon containing 2-12 carbon atoms. The “alkynyl” group contains at least one triple bond in the chain. Examples of alkynyl groups include ethynyl, propargyl, n-butynyl, iso-butynyl, pentynyl, or hexynyl.

It is understood that for example the terms “alkenyl”, “alkoxy”, “alkyl”, “alkynyl”, “haloalkyl”, “haloalkoxy” and “cycloalkyl” can be further defined by the numbers of carbons they contain, such as for example the term “C₁-C₆ alkoxy” refers to an alkoxy group as defined above containing 1-6 carbon atoms.

The term “aromatic” means a planar ring having 4n+2 electrons in a conjugated system. As used herein, “conjugated system” means a system of connected p-orbitals with delocalized electrons, and the system may include lone electron pairs.

The term “aryl” unless otherwise specifically defined refers to cyclic, aromatic hydrocarbon groups that have 1 to 3 aromatic rings, including monocyclic, or bicyclic groups such as phenyl, biphenyl, or naphthyl. Where containing two aromatic rings (bicyclic, etc.), the aromatic rings of the aryl group may be joined at a single point (e.g., biphenyl), or fused (e.g., naphthyl). Furthermore, when containing two fused rings the aryl groups herein defined may have one, or more saturated, or partially unsaturated ring fused with a fully unsaturated aromatic ring. Exemplary ring systems of these aryl groups include, but are not limited to, phenyl, biphenyl, naphthyl, anthracenyl, phenalenyl, phenanthrenyl, indanyl, indenyl, tetrahydronaphthalenyl, and tetrahydrobenzoannulenyl.

The term “carrier” encompasses carriers, excipients, and diluents, and means a material, composition, or vehicle, such as a liquid, or solid filler, diluent, excipient, solvent, or encapsulating material, involved in carrying, or transporting a pharmaceutical agent from one organ, or portion of the body, to another organ, or portion of the body of a subject.

The term “cyano” means a substituent having a carbon atom joined to a nitrogen atom by a triple bond, i.e., C≡N.

The term “cycloalkyl” means mono, or polycyclic saturated carbon rings containing 3-18 carbon atoms, preferably 3-10 carbon atoms. Examples of cycloalkyl groups include, without limitation, cyclopropyl, cyclobutyl, cyclopentyl, cyclohexyl, cycloheptanyl, cyclooctanyl, norbornyl, norborenyl, bicyclo[2.2.2]octanyl, and bicyclo[2.2.2]octenyl.

The term “disorder” means, and is used interchangeably with, the terms disease, condition, or illness, unless otherwise indicated.

The term “haloalkoxy” refers to an alkoxy group, as defined herein, which is substituted with one, or more halogen. Examples of haloalkoxy groups include, but are not limited to, trifluoromethoxy, difluoromethoxy, pentafluoroethoxy, and trichloromethoxy.

The term “haloalkyl” refers to an alkyl group, as defined herein, which is substituted with one, or more halogen. Examples of haloalkyl groups include, but are not limited to, trifluoromethyl, difluoromethyl, pentafluoroethyl, and trichloromethyl.

The term “halogen” or “halo” refers to fluorine, chlorine, bromine, or iodine.

The term “heteroaryl” unless otherwise specifically defined means a monovalent monocyclic, or a polycyclic aromatic radical of 5 to 24 ring atoms, preferably 5 to 10 ring atoms, containing one, or more ring heteroatoms selected from N, O, S, P, or B, preferably 1, 2, 3, or 4 ring heteroatoms selected from N, O, or S, the remaining ring atoms being C. A polycyclic aromatic radical includes two, or more fused rings, and may further include two, or more spiro-fused rings, e.g., bicyclic, tricyclic, tetracyclic, and the like. Unless otherwise specifically defined, “fused” means two rings sharing two ring atoms. Unless otherwise specifically defined, “spiro-fused” means two rings sharing one ring atom. Heteroaryl as herein defined also means a bicyclic heteroaromatic group wherein the heteroatom is selected from N, O, S, P, or B, preferably N, O, or S. Heteroaryl as herein defined also means a tricyclic heteroaromatic group containing one, or more ring heteroatoms selected from N, O, S, P, or B, preferably N, O, or S. Heteroaryl as herein defined also means a tetracyclic heteroaromatic group containing one, or more ring heteroatoms selected from N, O, S, P, or B, preferably N, O, or S. Examples of heteroaromatic groups include, but are not limited to, furyl, thienyl, pyrrolyl, pyridyl, pyrazolyl, pyrimidinyl, imidazolyl, isoxazolyl, oxazolyl, oxadiazolyl, pyrazinyl, indolyl, thiophen-2-yl, quinolyl, benzopyranyl, isothiazolyl, thiazolyl, thiadiazole, indazole, benzimidazolyl, thieno[3,2-b]thiophene, triazolyl, triazinyl, imidazo[1,2-b]pyrazolyl, furo[2,3-c]pyridinyl, imidazo[1,2-a]pyridinyl, indazolyl, pyrrolo[2,3-c]pyridinyl, pyrrolo[3,2-c]pyridinyl, pyrazolo[3,4-c]pyridinyl, thieno[3,2-c]pyridinyl, thieno[2,3-c]pyridinyl, thieno[2,3-b]pyridinyl, benzothiazolyl, indolyl, indolinyl, indolinonyl, dihydrobenzothiophenyl, dihydrobenzofuranyl, benzofuranyl, chromanyl, thiochromanyl, tetrahydroquinolinyl, dihydrobenzothiazinyl, quinolinyl, isoquinolinyl, 1,6-naphthyridinyl, benzo[de]isoquinolinyl, pyrido[4,3-b][1,6]naphthyridinyl, thieno[2,3-b]pyrazinyl, quinazolinyl, tetrazolo[1,5-a]pyridinyl, [1,2,4]triazolo[4,3-a]pyridinyl, isoindolyl, pyrrolo[2,3-b]pyridinyl, pyrrolo[3,4-b]pyridinyl, pyrrolo[3,2-b]pyridinyl, imidazo[5,4-b]pyridinyl, pyrrolo[1,2-a]pyrimidinyl, tetrahydro pyrrolo[1,2-a]pyrimidinyl, 3,4-dihydro-2H-1-pyrrolo[2,1-b]pyrimidine, dibenzo[b,d]thiophene, pyridin-2-one, furo[3,2-c]pyridinyl, furo[2,3-c]pyridinyl, 1H-pyrido[3,4-b][1,4]thiazinyl, benzooxazolyl, benzoisoxazolyl, furo[2,3-b]pyridinyl, benzothiophenyl, 1,5-naphthyridinyl, furo[3,2-b]pyridine, [1,2,4]triazolo[1,5-a]pyridinyl, benzo[1,2,3]triazolyl, imidazo[1,2-a]pyrimidinyl, [1,2,4]triazolo[4,3-b]pyridazinyl, benzo[c][1,2,5]thiadiazolyl, benzo[c][1,2,5]oxadiazole, 1,3-dihydro-2H-benzo[d]imidazol-2-one, 3,4-dihydro-2H-pyrazolo[1,5-b][1,2]oxazinyl, 4,5,6,7-tetrahydropyrazolo[1,5-a]pyridinyl, thiazolo[5,4-d]thiazolyl, imidazo[2,1-b][1,3,4]thiadiazolyl, thieno[2,3-b]pyrrolyl, and 3H-indolyl. Furthermore, when containing two, or more fused rings, the heteroaryl groups defined herein may have one, or more saturated, or partially unsaturated ring fused with one, or more fully unsaturated aromatic ring. In heteroaryl ring systems containing more than two fused rings, a saturated, or partially unsaturated ring may further be fused with a saturated, or partially unsaturated ring described herein. Furthermore, when containing three, or more fused rings, the heteroaryl groups defined herein may have one, or more saturated, or partially unsaturated ring spiro-fused. Any saturated, or partially unsaturated ring described herein is optionally substituted with one, or more oxo. Exemplary ring systems of these heteroaryl groups include, for example, indolinyl, indolinonyl, dihydrobenzothiophenyl, dihydrobenzofuran, chromanyl, thiochromanyl, tetrahydroquinolinyl, dihydrobenzothiazine, 3,4-dihydro-1H-isoquinolinyl, 2,3-dihydrobenzofuranyl, benzofuranonyl, oxindolyl, indolyl, 1,6-dihydro-7H-pyrazolo[3,4-c]pyridin-7-onyl, 7,8-dihydro-6H-pyrido[3,2-b]pyrrolizinyl, 8H-pyrido[3,2-b]pyrrolizinyl, 1,5,6,7-tetrahydrocyclopenta[b]pyrazolo[4,3-e]pyridinyl, 7,8-dihydro-6H-pyrido[3,2-b]pyrrolizinyl, pyrazolo[1,5-a]pyrimidin-7(4H)-onyl, 3,4-dihydropyrazino[1,2-a]indol-1(2H)-onyl, benzo[c][1,2]oxaborol-1(3H)-olyl, 6,6a,7,8-tetrahydro-9H-pyrido[2,3-b]pyrrolo[1,2-d][1,4]oxazin-9-onyl, and 6a′,7′-dihydro-6′H,9′H-spiro[cyclopropane-1,8′-pyrido[2,3-b]pyrrolo[1,2-d][1,4]oxazin]-9′-onyl.

The term “5-member heteroaryl” unless otherwise specifically defined means a monovalent monocyclic aromatic radical of 5 ring atoms, containing one, or more ring heteroatoms selected from N, O, S, P, or B, preferably 1, 2, 3, or 4 ring heteroatoms selected from N, O, or S, the remaining ring atoms being C. Exemplary 5-member heteroaryl groups include, but are not limited to, furyl, thiophenyl, pyrrolyl, pyrazolyl, imidazolyl, isoxazolyl, oxazolyl, oxadiazolyl, isothiazolyl, thiazolyl, thiadiazole, triazolyl and tetrazolyl.

The term “6-member heteroaryl” unless otherwise specifically defined means a monovalent monocyclic aromatic radical of 6 ring atoms, containing one, or more ring heteroatoms selected from N, O, S, P, or B, preferably 1, 2, 3, or 4 ring heteroatoms selected from N, O, or S, the remaining ring atoms being C. Exemplary 6-member heteroaryl groups include, but are not limited to, pyridyl, pyridazinyl, pyrimidinyl, pyrazinyl, or triazinyl.

The term “heterocyclyl”, “heterocycle”, or “heterocycloalkyl” means mono, or polycyclic rings containing 3-24 atoms, preferably 3-10 atoms, which include carbon, and one, or more heteroatoms selected from N, O, S, P, or B, preferably 1, 2, 3, or 4 heteroatoms selected from N, O, and S, and wherein the rings are not aromatic. Examples of heterocyclyl rings include, but are not limited to, oxetanyl, azetidinyl, tetrahydrofuranyl, tetrahydropyranyl, pyrrolidinyl, oxazolinyl, oxazolidinyl, thiazolinyl, thiazolidinyl, pyranyl, thiopyranyl, tetrahydropyranyl, dioxalinyl, piperidinyl, morpholinyl, thiomorpholinyl, thiomorpholinyl S-oxide, thiomorpholinyl S-dioxide, piperazinyl, azepinyl, oxepinyl, diazepinyl, tropanyl, oxazolidinonyl, and homotropanyl.

The term “isomers” refers to compounds that have the same molecular formula but differ in the nature or sequence of bonding of their atoms or the arrangement of their atoms in space. Isomers that differ in the arrangement of their atoms in space are termed “stereoisomers”. Stereoisomers that are not mirror images of one another are termed “diastereomers” and those that are non-superimposable mirror images of each other are termed “enantiomers”. When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomers or as a mixture thereof. A mixture containing equal proportions of the enantiomers is called a “racemic mixture”.

The term “modulate”, “modulation”, or “modulating” refers to a biological activity of a compound, or substrate that inhibits and/or activates PI3K.

The term “patient”, or “subject” is a mammal, e.g., a human, mouse, rat, guinea pig, dog, cat, horse, cow, pig, or non-human primate, such as a monkey, chimpanzee, baboon, or rhesus. Preferably, the mammal is human.

The term “therapeutically effective amount” when used in connection with a compound refers to the amount or dose of the compound which upon single or multiple dose administration to the patient, provides the desired effect in the patient under diagnosis or treatment. An effective amount can be determined by one skilled in the art by the use of known techniques and by observing results obtained under analogous circumstances. In determining the effective amount for a patient, a number of factors are considered by the attending diagnostician, including, but not limited to: the species of patient; its size, age, and general health; the specific disease or disorder involved; the degree of or involvement or the severity of the disease or disorder; the response of the individual patient; the particular compound administered; the mode of administration; the bioavailability characteristics of the preparation administered; the dose regimen selected; the use of concomitant medication; and other relevant circumstances.

The term “treating” with regard to a subject, includes restraining, slowing, stopping, or reversing the progression or severity of an existing symptom or disorder.

### Compounds of the Present Invention

In one aspect, the present invention provides compounds of Formula (I), or pharmaceutically acceptable salts thereof:

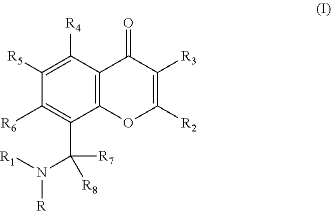

wherein R, R₁, R₂, R₃, R₄, R₅, R₆, R₇, and R₈, are as defined in the Summary for Formula (I).

In a compound of Formula (I), or pharmaceutically acceptable salts thereof,
R′ is C₁-C₃ haloalkyl, C₁-C₃ alkoxy, —NO₂, —N(R₁₁)—CO₂C₁-C₃ alkyl, —N(R₁₁)—SO₂C₁-C₃ alkyl, —SO₂NR₁₁R₁₁, —SO₂N(R₁₁)—CO—C₁-C₃ alkyl, —C(═N—OH)—NH₂, —CN, —CO₂C₁-C₃ alkyl, —CONR₁₁R₁₂, —CON(R)—(CH₂)ₙ—R₁₃, or a group of the formula

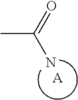

wherein ring A is pyrrolidine optionally substituted with a —CN; or
R′ is selected from oxetane, pyrrolidine, tetrahydrofuran, pyrrole, furan, thiophene, pyrazole, imidazole, isoxazole, oxazole, isothiazole, thiazole, triazole, oxadiazole, thiadiazole, tetrazole, pyridine, pyridazine, pyrimidine, pyrazine, or triazine; each of which is optionally substituted with one to three substituents independently selected from oxo, —OH, —NR₁₁R₁₁, —N(R₁₁)—CO—R₁₁, —N(R₁₁)—CN, —OR₁₁, —CN, C₁-C₆ haloalkyl or C₁-C₆ alkyl optionally substituted with an aryl, a 5-member heteroaryl or a 6-member heteroaryl.

In a compound of Formula (I), or pharmaceutically acceptable salts thereof, R and R₁ together with the nitrogen to which they are attached form an optionally substituted bicyclic ring selected from dihydrobenzimidazolone, dihydroindazolone, indolinone or quinazolinone; wherein the optionally substituted bicyclic ring is optionally substituted with one to three substituents each independently selected from oxo, —CN, halogen, C₁-C₆ alkyl, C₁-C₆ haloalkyl, —OH or C₁-C₆ alkoxy; or
R is —H or C₁-C₃ alkyl; and
R₁ is an optionally substituted bicyclic ring selected from isobenzofuranone, benzofuranone, isoindolinone, indolinone, quinazolinone, 3,4-dihydro-2H-isoquinolin-1-one, 2H-isoquinolin-1-one, imidaza[1,2-a]pyridine, or benzothiazolone; wherein the optionally substituted bicyclic ring is optionally substituted with one to three substituents each independently selected from oxo, —CN, halogen, C₁-C₆ alkyl, C₁-C₆ haloalkyl, —OH or C₁-C₆ alkoxy; or in some embodiments, R₁ is an optionally substituted bicyclic ring selected from isobenzofuranone, benzofuranone, isoindolinone, indolinone, quinazolinone, or benzothiazolone; wherein the optionally substituted bicyclic ring is optionally substituted with one to three substituents each independently selected from oxo, —CN, halogen, C₁-C₆ alkyl, C₁-C₆ haloalkyl, —OH or C₁-C₆ alkoxy; or
R₁ is a group of the formula:

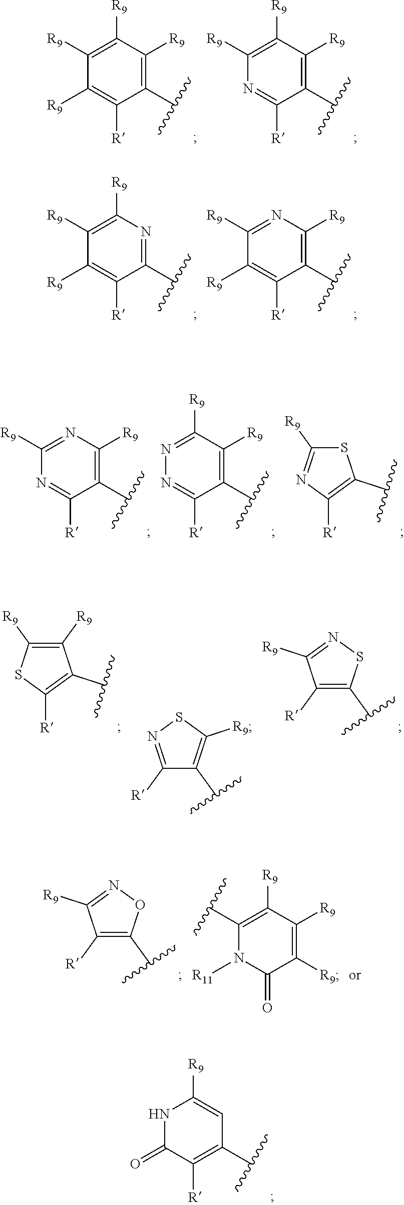

or
R₁ is a group of the formula:

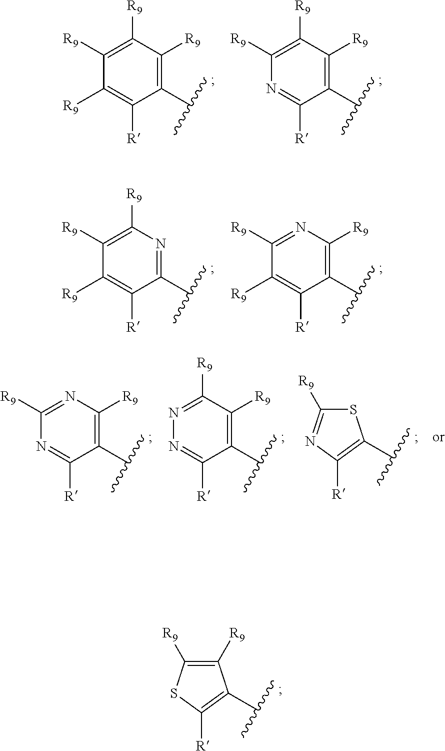

R′ is hydrogen, halogen, C₁-C₃ alkyl, C₁-C₃ haloalkyl, C₁-C₃ hydroxyalkyl, C₁-C₃ alkoxy, C₁-C₃ haloalkoxy, —OH, —CH(OH)—CH₂OH, —CH(OH)C₁-C₃ haloalkyl, —CO—CH₂OH, C₃-C₆ cycloalkyl, —NO₂, —NR₁₁R₁₁, —N(R₁₁)—CO₂C₁-C₃ alkyl, —N(R₁₁)—SO₂C₁-C₃ alkyl, —N(R₁₁)—SO₂R₁₅, —SO₂C₁-C₃ alkyl, —SOC₁-C₃ alkyl, —SO₂NR₁₁R₁₁, —SO₂N(R₁₁)—CO—C₁-C₃ alkyl, —SO₂N(R₁₁)—CN, —SO₂N(R₁₁)(R₁₃)—C(═N—OH)—NH₂, —CN, —CO₂C₁-C₃ alkyl, —CONR₁₁R₁₂, —CON(R)—(CH₂)ₙ—R₁₃, —CO—SR₁₂, —CO—NHSO₂R₁₆, —COCH═SOR₁₁(R₁₁), or —COCH₂CN, or a group of the formula:

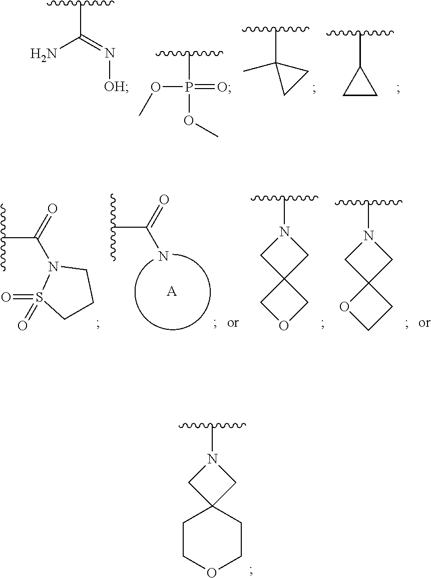

wherein ring A is pyrrolidine optionally substituted with a —CN; or
R′ is C₁-C₃ haloalkyl, C₁-C₃ alkoxy, —NO₂, —N(R)—CO₂C₁-C₃ alkyl, —N(R)—SO₂C₁-C₃ alkyl, —SO₂NR₁₁R₁₁, —SO₂N(R)—CO—C₁-C₃ alkyl, —C(═N—OH)—NH₂, —CN, —CO₂C₁-C₃ alkyl, —CONR₁₁R₁₂, —CON(R)—(CH₂)ₙ—R₁₃, or a group of the formula

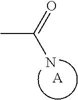

wherein ring A is pyrrolidine optionally substituted with a —CN; or
R′ is selected from oxetane, azetidine, pyrrolidine, tetrahydrofuran, morpholine, thiomorpholine, piperidine, piperazine, pyrrole, furan, thiophene, pyrazole, imidazole, isoxazole, oxazole, isothiazole, thiazole, triazole, oxadiazole, 1,2,4-oxadiazolin-5-one, thiadiazole, tetrazole, phenyl, pyridine, pyridazine, pyrimidine, pyrazine, or triazine; each of which is optionally substituted with one to three substituents independently selected from oxo, —OH, —NR₁₁R₁₁, —N(R₁₁C(O)—R₁₁, —N(R₁₁)—CN, —OR₁₁, —CN, halogen, morpholine, oxetane, C₁-C₆ haloalkyl or C₁-C₆ alkyl optionally substituted with an aryl, a 5-member heteroaryl or a 6-member heteroaryl; or
R′ is selected from oxetane, pyrrolidine, tetrahydrofuran, pyrrole, furan, thiophene, pyrazole, imidazole, isoxazole, oxazole, isothiazole, thiazole, triazole, oxadiazole, thiadiazole, tetrazole, pyridine, pyridazine, pyrimidine, pyrazine, or triazine; each of which is optionally substituted with one to three substituents independently selected from oxo, —OH, —NR₁₁R₁₁, —N(R₁₁)—CO—R₁₁, —N(R₁₁)—CN, —OR₁₁, —CN, C₁-C₆ haloalkyl or C₁-C₆ alkyl optionally substituted with an aryl, a 5-member heteroaryl or a 6-member heteroaryl; or
R′ is a group of the formula:

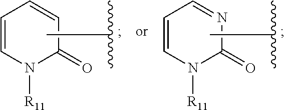

R₂ is a group of the formula:

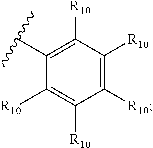

or
R₂ is a group of the formula:

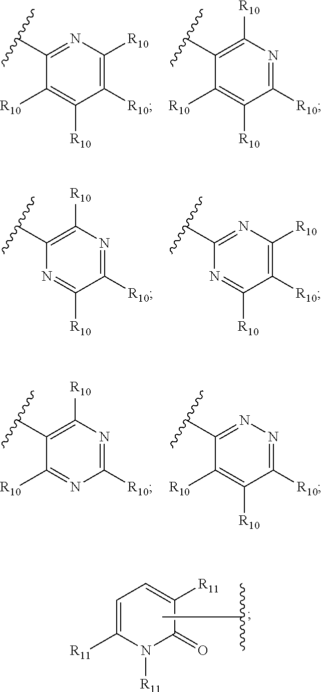

or
R₂ is a group of the formula:

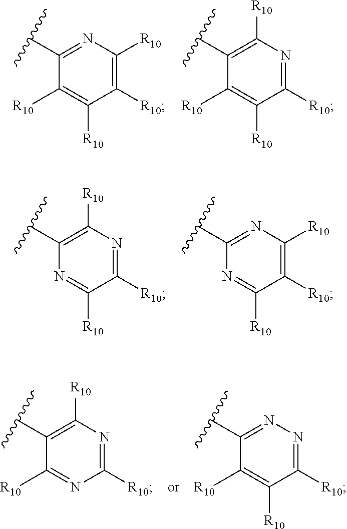

or
R₂ is an optionally substituted 5-member ring heteroaryl selected from pyrrole, furan, thiophene, pyrazole, isoxazole, isothiazole, imidazole, oxazole, thiazole, triazole, tetrazole, oxadiazole, and thiadiazole; wherein the optionally substituted 5-member ring heteroaryl is optionally substituted with one to three substituents each independently selected from —CN, halogen, C₁-C₆ haloalkyl, C₁-C₆ alkoxy, C₁-C₆ haloalkoxy, —SO₂R₁₁, —CO₂C₁-C₃ alkyl, —C(O)NR₁₁R₁₁, —OH, —NR₁₁R₁₁, —NR₁₁CO₂R₁₁, —CD₃, an optionally substituted C₁-C₆ alkyl, an optionally substituted C₂-C₆ alkenyl, an optionally substituted C₂-C₆ alkynyl, an optionally substituted C₃-C₅ cycloalkyl, an optionally substituted heterocycle selected from pyrrolidine, pyrrolidinone, piperidine, tetrahydropyran, oxetane, azetidine, or morpholine, an optionally substituted phenyl, an optionally substituted 1,3-benzodioxole, an optionally substituted 2,3-dihydro-1,4-benzodioxine, or an optionally substituted heteroaryl selected from pyridine, pyrimidine, pyridazine, pyrazine, pyrazole, isoxazole, isothiazole, imidazole, oxazole, or thiazole; wherein the optionally substituted C₁-C₆ alkyl, C₂-C₆ alkenyl, or C₂-C₆ alkynyl is each optionally substituted with a —CN, —OH, oxetanyl, C₁-C₃ alkoxy, —CH₂CH(OH)CH(OH)R₁₁, or —CONR₁₁R₁₁; the optionally substituted C₃-C₅ cycloalkyl, phenyl, 1,3-benzodioxole, 2,3-dihydro-1,4-benzodioxine, heterocycle or heteroaryl is each optionally substituted with one to three substituents each independently selected from halogen, C₁-C₃ alkyl, C₁-C₃ haloalkyl, C₁-C₃ alkoxy, C₁-C₃ haloalkoxy, —C(O)(CH₂)ₙOR₁₁, C(O)C(CH₃)₂OH, —SO₂R₁₁, —NR₁₁R₁₁, —OH or —CN; or R₂ is an optionally substituted 5-member ring heteroaryl selected from pyrrole, furan, thiophene, pyrazole, isoxazole, isothiazole, imidazole, oxazole, thiazole, triazole, tetrazole, oxadiazole, and thiadiazole; wherein the optionally substituted 5-member ring heteroaryl is optionally substituted with one to three substituents each independently selected from —CN, halogen, C₁-C₆ haloalkyl, C₁-C₆ alkoxy, C₁-C₆ haloalkoxy, —SO₂R₁₁, —CO₂C₁-C₃ alkyl, —CONR₁₁R₁₁, —OH, —NR₁₁R₁₁, —NR₁₁CO₂R₁₁, an optionally substituted C₁-C₆ alkyl, an optionally substituted C₂-C₆ alkenyl, an optionally substituted C₂-C₆ alkynyl, an optionally substituted C₃-C₅ cycloalkyl, an optionally substituted heterocycle selected from pyrrolidine, pyrrolidinone, piperidine or morpholine, an optionally substituted phenyl, an optionally substituted 1,3-benzodioxole, an optionally substituted 2,3-dihydro-1,4-benzodioxine, or an optionally substituted heteroaryl selected from pyridine, pyrimidine, pyridazine, pyrazine, pyrazole, isoxazole, isothiazole, imidazole, oxazole, or thiazole; wherein the optionally substituted C₁-C₆ alkyl, C₂-C₆ alkenyl, or C₂-C₆ alkynyl is each optionally substituted with a —CN, —OH, oxetanyl, C₁-C₃ alkoxy, or —CONR₁₁R₁₁; the optionally substituted C₃-C₅ cycloalkyl, phenyl, 1,3-benzodioxole, 2,3-dihydro-1,4-benzodioxine, heterocycle or heteroaryl is each optionally substituted with one to three substituents each independently selected from halogen, C₁-C₃ alkyl, C₁-C₃ haloalkyl, C₁-C₃ alkoxy, C₁-C₃ haloalkoxy, —SO₂R₁₁, —NR₁₁R₁₁, —OH or —CN; or
R₂ is an optionally substituted bicyclic ring selected from 1,3-benzodioxole, 2,3-dihydro-1,4-benzodioxine, indole, indazole, isoindazole, isoindolin-1-one, indolin-2-one, benzoxazole, benzotriazole, benzo[d]oxazol-2(3H)-one, 1,3-dihydro-2H-pyrrolo[2,3-b]pyridin-2-one, 6,7-dihydro-4H-pyrazolo[5,1-c][1,4]oxazine, 4,5,6,7-tetrahydropyrazolo[1,5-a]pyrazine, or 2,3-dihydro-[1,4]dioxino[2,3-b]pyridine, imidazo[1,2-a]pyridine, pyrazolo[4,3-b]pyridine, pyrazolo[3,4-b]pyridine, pyrazolo[3,4-c]pyridine, pyrazolo[1,5-a]pyrimidine, or an optionally substituted bicyclic heteroaryl of 8 to 10 ring atoms containing 1, 2, 3, 4, or 5 ring heteroatoms independently selected from N, O, or S; wherein the optionally substituted bicyclic ring is optionally substituted with one to three substituents each independently selected from halogen and C₁-C₆ alkyl; the optionally substituted bicyclic heteroaryl is optionally substituted with one to three substituents each independently selected from —CN, halogen, C₁-C₆ haloalkyl, C₁-C₆ alkoxy, C₁-C₆ haloalkoxy, —SO₂R₁₁, —CO₂CH, —CO₂C₁-C₃ alkyl, —CONR₁₁R₁₁, —NR₁₁R₁₁, —NR₁₁CO₂R₁₁, —OH, an optionally substituted C₁-C₆ alkyl, an optionally substituted C₂-C₆ alkenyl, an optionally substituted C₂-C₆ alkynyl, an optionally substituted C₃-C₅ cycloalkyl, an optionally substituted heterocycle selected from pyrrolidine, pyrrolidinone, piperidine or morpholine, an optionally substituted phenyl, an optionally substituted 1,3-benzodioxole, an optionally substituted 2,3-dihydro-1,4-benzodioxine, or an optionally substituted heteroaryl selected from pyridine, pyrimidine, pyridazine, pyrazine, pyrazole, isoxazole, isothiazole, imidazole, oxazole, or thiazole; wherein the optionally substituted C₁-C₆ alkyl, C₂-C₆ alkenyl, or C₂-C₆ alkynyl is each optionally substituted with a —CN, —OH, oxetanyl, C₁-C₃ alkoxy, —C(O)NR₁₁R₁₁ or phenyl; the optionally substituted C₃-C₅ cycloalkyl, phenyl, 1,3-benzodioxole, 2,3-dihydro-1,4-benzodioxine, heterocycle or heteroaryl is each optionally substituted with one to three substituents each independently selected from halogen, C₁-C₃ alkyl, C₁-C₃ haloalkyl, C₁-C₃ alkoxy, C₁-C₃ haloalkoxy, —SO₂R₁₁, —NR₁₁R₁₁, —OH or —CN; or
R₂ is an optionally substituted bicyclic ring selected from 1,3-benzodioxole, 2,3-dihydro-1,4-benzodioxine, isoindolin-1-one, indolin-2-one, benzo[d]oxazol-2(3H)-one, 1,3-dihydro-2H-pyrrolo[2,3-b]pyridin-2-one, or 2,3-dihydro-[1,4]dioxino[2,3-b]pyridine, or an optionally substituted bicyclic heteroaryl of 8 to 10 ring atoms containing 1, 2, 3, 4, or 5 ring heteroatoms independently selected from N, O, or S; wherein the optionally substituted bicyclic ring is optionally substituted with one to three substituents each independently selected from halogen and C₁-C₆ alkyl; the optionally substituted bicyclic heteroaryl is optionally substituted with one to three substituents each independently selected from —CN, halogen, C₁-C₆ haloalkyl, C₁-C₆ alkoxy, C₁-C₆ haloalkoxy, —SO₂R₁₁, —CO₂C₁-C₃ alkyl, —CONR₁₁R₁₁, —NR₁₁R₁₁, —NR₁₁CO₂R₁₁, —OH, an optionally substituted C₁-C₆ alkyl, an optionally substituted C₂-C₆ alkenyl, an optionally substituted C₂-C₆ alkynyl, an optionally substituted C₃-C₅ cycloalkyl, an optionally substituted heterocycle selected from pyrrolidine, pyrrolidinone, piperidine or morpholine, an optionally substituted phenyl, an optionally substituted 1,3-benzodioxole, an optionally substituted 2,3-dihydro-1,4-benzodioxine, or an optionally substituted heteroaryl selected from pyridine, pyrimidine, pyridazine, pyrazine, pyrazole, isoxazole, isothiazole, imidazole, oxazole, or thiazole; wherein the optionally substituted C₁-C₆ alkyl, C₂-C₆ alkenyl, or C₂-C₆ alkynyl is each optionally substituted with a —CN, —OH, oxetanyl, C₁-C₃ alkoxy, —CONR₁₁R₁₁ or phenyl; the optionally substituted C₃-C₅ cycloalkyl, phenyl, 1,3-benzodioxole, 2,3-dihydro-1,4-benzodioxine, heterocycle or heteroaryl is each optionally substituted with one to three substituents each independently selected from halogen, C₁-C₃ alkyl, C₁-C₃ haloalkyl, C₁-C₃ alkoxy, C₁-C₃ haloalkoxy, —SO₂R₁₁, —NR₁₁R₁₁, —OH or —CN; or
R₂ is a cyclopropyl, cyclobutyl, cyclopentyl, or cyclohexyl, each optionally substituted with one to three R₁₀ substituents, or R₂ is a group of the formula:

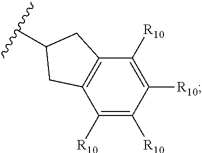

each R₁₀ is independently —H, —CN, halogen, C₁-C₆ haloalkyl, C₁-C₆ alkoxy, C₁-C₆ haloalkoxy, —SO₂R₁₁, —SONR₁₁R₁₁, —CO₂H, —CO₂C₁-C₃ alkyl, —C(O)NR₁₁R₁₂, —NR₁₁R₁₁, —NR₁₁—CO₂R₁₁, —N(R₁₁)COR₁₁, —OH, an optionally substituted C₁-C₆ alkyl, an optionally substituted C₂-C₆ alkenyl, an optionally substituted C₂-C₆ alkynyl, an optionally substituted C₃-C₅ cycloalkyl, an optionally substituted heterocycle selected from pyrrolidine, pyrrolidinone, piperidine or morpholine, an optionally substituted phenyl, an optionally substituted 1,3-benzodioxole, an optionally substituted 2,3-dihydro-1,4-benzodioxine, or an optionally substituted heteroaryl selected from pyrazole, isoxazole, isothiazole, imidazole, oxazole, thiazole, or pyridine; or
each R₁₀ is independently —H, —CN, halogen, C₁-C₆ haloalkyl, C₁-C₆ alkoxy, C₁-C₆ haloalkoxy, —SO₂R₁₁, —CO₂C₁-C₃ alkyl, —CONR₁₁R₁₁, —NR₁₁R₁₁, —NR₁₁—CO₂R₁₁, —OH, an optionally substituted C₁-C₆ alkyl, an optionally substituted C₂-C₆ alkenyl, an optionally substituted C₂-C₆ alkynyl, an optionally substituted C₃-C₅ cycloalkyl, an optionally substituted heterocycle selected from pyrrolidine, pyrrolidinone, piperidine or morpholine, an optionally substituted phenyl, an optionally substituted 1,3-benzodioxole, an optionally substituted 2,3-dihydro-1,4-benzodioxine, or an optionally substituted heteroaryl selected from pyrazole, isoxazole, isothiazole, imidazole, oxazole, or thiazole; wherein the optionally substituted C₁-C₆ alkyl, C₂-C₆ alkenyl, or C₂-C₆ alkynyl is each optionally substituted with a —CN, —OH, oxetanyl, C₁-C₃ alkoxy, or —CONR₁₁R₁₁; the optionally substituted C₃-C₅ cycloalkyl, phenyl, 1,3-benzodioxole, 2,3-dihydro-1,4-benzodioxine, heterocycle or heteroaryl is each optionally substituted with one to three substituents each independently selected from halogen, C₁-C₃ alkyl, C₁-C₃ haloalkyl, C₁-C₃ alkoxy, C₁-C₃ haloalkoxy, —SO₂R₁₁, —NR₁₁R₁₁, —OH or —CN;
each R₁₁ is independently —H, C₁-C₃ alkyl, C₃-C₇ cycloalkyl, or C₁-C₃ haloalkyl;
R₁₂ is —H, optionally substituted C₁-C₃ alkyl, C₃-C₆ cycloalkyl, —SO₂C₁-C₃ alkyl, —SO₂C₁-C₃ haloalkyl, —SO₂NR₁₁R₁₁, —NR₁₁R₁₁, —OR₁₁, —O—CH₂—CH(OH)—CH₂OH, —CN, oxetane, tetrahydrofuran, an aryl, a 5-member heteroaryl optionally substituted with a methyl, a 6-member heteroaryl, or a group of the formula

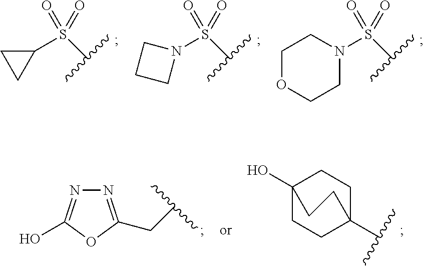 [SMILES: *S(=O)(=O)C1CC1 ; *S(=O)(=O)N1CCC1 ; *S(=O)(=O)N1CCOCC1 ; *Cc1nnc(O)o1 ; *C12CCC(O)(CC1)CC2]

wherein the optionally substituted C₁-C₃ alkyl is optionally substituted with —OH, a C₃-C₆ cycloalkyl, oxetane, tetrahydrofuran, an aryl, a 5-member heteroaryl, a 6-member heteroaryl or an indole; and R₁₃ is —NR₁₁R₁₁, —OR₁₁, oxetane or tetrahydrofuran.

In a compound of Formula (I), or pharmaceutically acceptable salts thereof, R′ is hydrogen, halogen, C₁-C₃ alkyl, —CH(OH)—CH₂OH, —CO—CH₂OH or C₃-C₆ cycloalkyl.

In a compound of Formula (I), or pharmaceutically acceptable salts thereof,
R is —H or C₁-C₃ alkyl;
R₁ is a group of the formula:

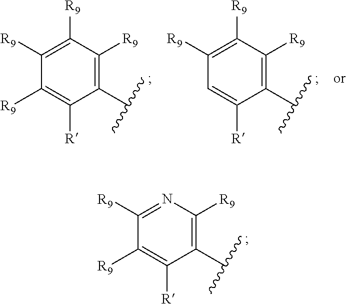

R′ is hydrogen, halogen, C₁-C₃ alkyl, —CH(OH)—CH₂OH, —CO—CH₂OH or C₃-C₆ cycloalkyl;
R₂ is a group of the formula:

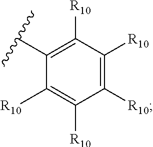

or
R₂ is a group of the formula:

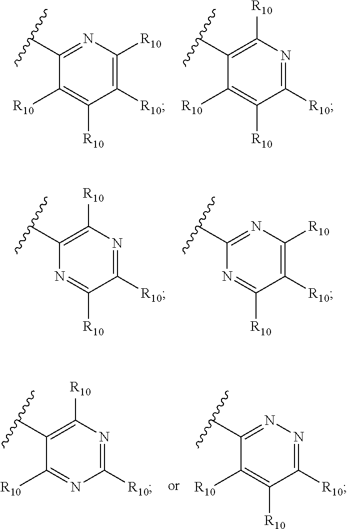

or
R₂ is an optionally substituted 5-member ring heteroaryl selected from pyrrole, furan, thiophene, pyrazole, isoxazole, isothiazole, imidazole, oxazole, thiazole, triazole, tetrazole, oxadiazole, and thiadiazole; wherein the optionally substituted 5-member ring heteroaryl is optionally substituted with one to three substituents each independently selected from —CN, halogen, C₁-C₆ haloalkyl, C₁-C₆ alkoxy, C₁-C₆ haloalkoxy, —SO₂R₁₁, —CO₂C₁-C₃ alkyl, —CONR₁₁R₁₁, —OH, —NR₁₁R₁₁, —NR₁₁CO₂R₁₁, an optionally substituted C₁-C₆ alkyl, an optionally substituted C₂-C₆ alkenyl, an optionally substituted C₂-C₆ alkynyl, an optionally substituted C₃-C₅ cycloalkyl, an optionally substituted heterocycle selected from pyrrolidine, pyrrolidinone, piperidine or morpholine, an optionally substituted phenyl, an optionally substituted 1,3-benzodioxole, an optionally substituted 2,3-dihydro-1,4-benzodioxine, or an optionally substituted heteroaryl selected from pyridine, pyrimidine, pyridazine, pyrazine, pyrazole, isoxazole, isothiazole, imidazole, oxazole, or thiazole; wherein the optionally substituted C₁-C₆ alkyl, C₂-C₆ alkenyl, or C₂-C₆ alkynyl is each optionally substituted with a —CN, —OH, oxetanyl, C₁-C₃ alkoxy, or —CONR₁₁R₁₁; the optionally substituted C₃-C₅ cycloalkyl, phenyl, 1,3-benzodioxole, 2,3-dihydro-1,4-benzodioxine, heterocycle or heteroaryl is each optionally substituted with one to three substituents each independently selected from halogen, C₁-C₃ alkyl, C₁-C₃ haloalkyl, C₁-C₃ alkoxy, C₁-C₃ haloalkoxy, —SO₂R₁₁, —NR₁₁R₁₁, —OH or —CN; or
R₂ is an optionally substituted bicyclic ring selected from 1,3-benzodioxole, 2,3-dihydro-1,4-benzodioxine, isoindolin-1-one, indolin-2-one, benzo[d]oxazol-2(3H)-one, 1,3-dihydro-2H-pyrrolo[2,3-b]pyridin-2-one, or 2,3-dihydro-[1,4]dioxino[2,3-b]pyridine, or an optionally substituted bicyclic heteroaryl of 8 to 10 ring atoms containing 1, 2, 3, 4, or 5 ring heteroatoms independently selected from N, O, or S; wherein the optionally substituted bicyclic ring is optionally substituted with one to three substituents each independently selected from halogen and C₁-C₆ alkyl; the optionally substituted bicyclic heteroaryl is optionally substituted with one to three substituents each independently selected from —CN, halogen, C₁-C₆ haloalkyl, C₁-C₆ alkoxy, C₁-C₆ haloalkoxy, —SO₂R₁₁, —CO₂C₁-C₃ alkyl, —CONR₁₁R₁₁, —NR₁₁R₁₁, —NR₁₁CO₂R₁₁, —OH, an optionally substituted C₁-C₆ alkyl, an optionally substituted C₂-C₆ alkenyl, an optionally substituted C₂-C₆ alkynyl, an optionally substituted C₃-C₅ cycloalkyl, an optionally substituted heterocycle selected from pyrrolidine, pyrrolidinone, piperidine or morpholine, an optionally substituted phenyl, an optionally substituted 1,3-benzodioxole, an optionally substituted 2,3-dihydro-1,4-benzodioxine, or an optionally substituted heteroaryl selected from pyridine, pyrimidine, pyridazine, pyrazine, pyrazole, isoxazole, isothiazole, imidazole, oxazole, or thiazole; wherein the optionally substituted C₁-C₆ alkyl, C₂-C₆ alkenyl, or C₂-C₆ alkynyl is each optionally substituted with a —CN, —OH, oxetanyl, C₁-C₃ alkoxy, —CONR₁₁R₁₁ or phenyl; the optionally substituted C₃-C₅ cycloalkyl, phenyl, 1,3-benzodioxole, 2,3-dihydro-1,4-benzodioxine, heterocycle or heteroaryl is each optionally substituted with one to three substituents each independently selected from halogen, C₁-C₃ alkyl, C₁-C₃ haloalkyl, C₁-C₃ alkoxy, C₁-C₃ haloalkoxy, —SO₂R₁₁, —NR₁₁R₁₁, —OH or —CN; or
R₂ is a cyclopropyl, cyclobutyl, cyclopentyl, or cyclohexyl, each optionally substituted with one to three R₁₀ substituents, or R₂ is a group of the formula:

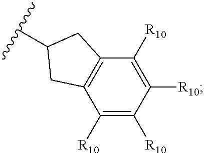

and
each R₁₀ is independently —H, —CN, halogen, C₁-C₆ haloalkyl, C₁-C₆ alkoxy, C₁-C₆ haloalkoxy, —SO₂R₁₁, —CO₂C₁-C₃ alkyl, —CONR₁₁R₁₁, —NR₁₁R₁₁, —NR₁₁—CO₂R₁₁, —OH, an optionally substituted C₁-C₆ alkyl, an optionally substituted C₂-C₆ alkenyl, an optionally substituted C₂-C₆ alkynyl, an optionally substituted C₃-C₅ cycloalkyl, an optionally substituted heterocycle selected from pyrrolidine, pyrrolidinone, piperidine or morpholine, an optionally substituted phenyl, an optionally substituted 1,3-benzodioxole, an optionally substituted 2,3-dihydro-1,4-benzodioxine, or an optionally substituted heteroaryl selected from pyrazole, isoxazole, isothiazole, imidazole, oxazole, thiazole or pyridine; wherein the optionally substituted C₁-C₆ alkyl, C₂-C₆ alkenyl, or C₂-C₆ alkynyl is each optionally substituted with a —CN, —OH, oxetanyl, C₁-C₃ alkoxy, —CO₂C₁-C₃ alkyl, —CONR₁₁R₁₁, —NR₁₁R₁₁ or phenyl; the optionally substituted C₃-C₅ cycloalkyl, phenyl, 1,3-benzodioxole, 2,3-dihydro-1,4-benzodioxine, heterocycle or heteroaryl is each optionally substituted with one to three substituents each independently selected from halogen, C₁-C₃ alkyl, C₁-C₃ haloalkyl, C₁-C₃ alkoxy, C₁-C₃ haloalkoxy, —SO₂R₁₁, —NR₁₁R₁₁, —OH or —CN.

In a compound of Formula (I), or pharmaceutically acceptable salts thereof, R₈ is —H.

In a further aspect, compounds of Formula (I), wherein R₈ is —H, have Formula (II), or pharmaceutically acceptable salts thereof:

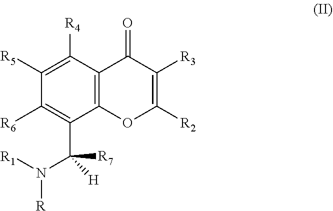

wherein R, R₁, R₂, R₃, R₄, R₅, R₆, and R₇, are as defined in the Summary for Formula (I).

In a compound of Formula (I), or (II), or pharmaceutically acceptable salts thereof, R and R₁ together with the nitrogen to which they are attached form an optionally substituted bicyclic ring selected from dihydrobenzimidazolone, dihydroindazolone, indolinone or quinazolinone; wherein the optionally substituted bicyclic ring is optionally substituted with one to three substituents each independently selected from oxo, —CN, halogen, C₁-C₆ alkyl, C₁-C₆ haloalkyl, —OH or C₁-C₆ alkoxy.

In a compound of Formula (I), or (II), or pharmaceutically acceptable salts thereof, R₁ is a group of a formula:

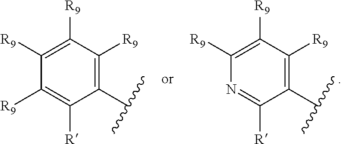

In a compound of Formula (I), or (II), or pharmaceutically acceptable salts thereof, R is —H.

In yet a further compound of Formula (I), or (II), or pharmaceutically acceptable salts thereof, R₄ is —H or halogen. Preferably R₄ is —H.

In a further aspect, compounds of Formula (I) or (II) have Formula (III), or pharmaceutically acceptable salts thereof:

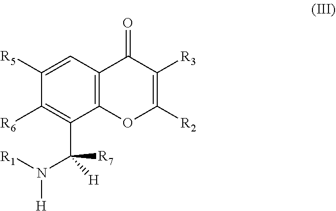

wherein R₁, R₂, R₃, R₅, R₆, and R₇ are as defined in the Summary for Formula (I) above.

In yet a further compound of Formula (I), (II) or (III), or pharmaceutically acceptable salts thereof, R₃ is —H, halogen, —CN, —C(CN)═CHOH, —N(H)(C₁-C₃ alkyl), —N(C₁-C₃ alkyl)₂, —N(H)(CH₂CH₂CO₂H), —C(O)—C₁-C₃ alkyl, C₁-C₆ alkyl, C₁-C₆ haloalkyl, C₁-C₆ hydroxyalkyl, C₃-C₅ cycloalkyl, an optionally substituted heterocycle of 3 to 5 ring atoms containing 1, 2, or 3 ring heteroatoms independently selected from N, O, or S, or an optionally substituted heteroaryl of 5 or 6 ring atoms containing 1, 2, or 3 ring heteroatoms independently selected from N, O, or S; wherein the optionally substituted heterocycle or heteroaryl is each optionally substituted with one to three substituents each independently selected from halogen, C₁-C₃ alkyl, or C₁-C₃ haloalkyl.

In yet a further compound of Formula (I), (II) or (III), or pharmaceutically acceptable salts thereof, R₃ is —H, halogen, —CN, C₁-C₆ alkyl, C₁-C₆ haloalkyl, oxetane, or isoxazole. Preferably R₃ is —H, —CN, C₁-C₃ alkyl, or C₁-C₃ haloalkyl. More preferably R₃ is —H, —CN, methyl, or trifluoromethyl. Most preferably R₃ is —H or methyl.

In yet a further compound of Formula (I), (II) or (III), or pharmaceutically acceptable salts thereof, R₅ is —H, halogen, C₁-C₃ alkyl or C₁-C₃ haloalkyl. Preferably R₅ is —H, halogen, methyl, or trifluoromethyl.

In yet a further compound of Formula (I), (II) or (III), or pharmaceutically acceptable salts thereof, R₆ is —H or halogen.

In yet a further compound of Formula (I), (II) or (III), or pharmaceutically acceptable salts thereof, R₇ is —CN, C₁-C₃ alkyl or C₁-C₃ haloalkyl. Preferably R₇ is —CN, methyl or trifluoromethyl. More preferably R₇ is methyl.

In yet a further compound of Formula (I), (II) or (III), or pharmaceutically acceptable salts thereof, R₁ is an optionally substituted bicyclic ring selected from isobenzofuranone, benzofuranone, isoindolinone, indolinone, quinazolinone, or benzothiazolone; wherein the optionally substituted bicyclic ring is optionally substituted with one to three substituents each independently selected from oxo, —CN, halogen, C₁-C₆ alkyl, C₁-C₆ haloalkyl, —OH or C₁-C₆ alkoxy.

In yet a further compound of Formula (I), (II) or (III), or pharmaceutically acceptable salts thereof, R₁ is a group of the formula

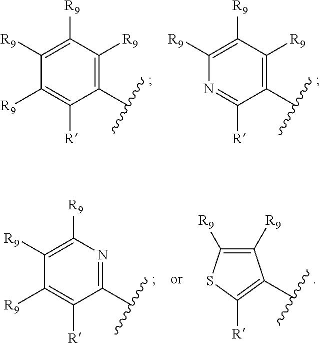

In yet a further compound of Formula (I), (II) or (III), or pharmaceutically acceptable salts thereof, R₁ is a group of the formula

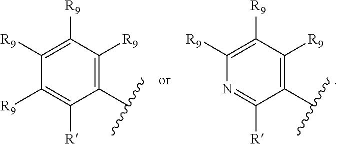

In yet a further compound of Formula (I), (II) or (III), or pharmaceutically acceptable salts thereof, each R₉ is independently —H, halogen, C₁-C₃ alkyl, or C₃-C₅ cycloalkyl, preferably each R₉ is independently —H, halogen, methyl, or cyclopropyl.

In yet a further compound of Formula (I), (II) or (III), or pharmaceutically acceptable salts thereof, each R₉ is independently —H, halogen, C₁-C₃ alkyl, or C₁-C₃ alkoxy.

In yet a further compound of Formula (I), (II) or (III), or pharmaceutically acceptable salts thereof, R′ is C₁-C₃ haloalkyl, C₁-C₃ alkoxy, —NO₂, —N(R₁₁)—CO₂C₁-C₃ alkyl, —N(R₁₁)—SO₂C₁-C₃ alkyl, —SO₂NR₁₁R₁₁, —SO₂N(R₁₁)—CO—C₁-C₃ alkyl, —C(═N—OH)—NH₂, —CN, —CO₂C₁-C₃ alkyl, —CONR₁₁R₁₂, —CON(R)—(CH₂)ₙ—R₁₃, or a group of the formula

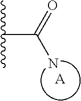

wherein ring A is pyrrolidine optionally substituted with a —CN.

In yet a further compound of Formula (I), (II) or (III), or pharmaceutically acceptable salts thereof, R′ is C₁-C₃ haloalkyl, C₁-C₃ alkoxy, —NO₂, —N(R)—CO₂C₁-C₃ alkyl, —N(R)—SO₂C₁-C₃ alkyl, —SO₂NR₁₁R₁₁, —SO₂N(R)—CO—C₁-C₃ alkyl, —C(═N—OH)—NH₂, —CN, —CONR₁₁R₁₂, —CON(R)—(CH₂)ₙ—R₁₃, or a group of the formula

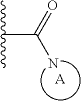

wherein ring A is pyrrolidine optionally substituted with a —CN.

In yet a further compound of Formula (I), (II) or (III), or pharmaceutically acceptable salts thereof, R′ is —SO₂NR₁₁R₁₁, —SO₂N(R₁₁)(R₁₃), —C(O)NR₁₁R₁₂, —C(O)N(R)—(CH₂)ₙ—R₁₃, —C(O)—NHSO₂R₁₆, or 1,2,4-oxadiazolin-5-one.

In yet a further compound of Formula (I), (II) or (III), or pharmaceutically acceptable salts thereof, R′ is selected from oxetane, pyrrolidine, tetrahydrofuran, morpholine, piperidine, piperazine, pyrrole, furan, thiophene, pyrazole, imidazole, isoxazole, oxazole, isothiazole, thiazole, triazole, oxadiazole, thiadiazole, tetrazole, pyridine, pyridazine, pyrimidine, pyrazine, or triazine; each of which is optionally substituted with one to three substituents independently selected from oxo, —NR₁₁R₁₁, —OR₁₁, —CN, C₁-C₆ haloalkyl or C₁-C₆ alkyl optionally substituted with phenyl.

In yet a further compound of Formula (I), (II) or (III), or pharmaceutically acceptable salts thereof, R′ is selected from oxetane, pyrrolidine, tetrahydrofuran, pyrrole, furan, thiophene, pyrazole, imidazole, isoxazole, oxazole, isothiazole, thiazole, triazole, oxadiazole, thiadiazole, tetrazole, pyridine, pyridazine, pyrimidine, pyrazine, or triazine; each of which is optionally substituted with one to three substituents independently selected from oxo, —NR₁₁R₁₁, —OR₁₁, —CN, C₁-C₆ haloalkyl or C₁-C₆ alkyl optionally substituted with phenyl

In yet a further compound of Formula (I), (II) or (III), or pharmaceutically acceptable salts thereof, R′ is pyrrole, furan, thiophene, pyrazole, isoxazole, oxazole, isothiazole, thiazole, imidazole, triazole, oxadiazole, thiadiazole, tetrazole, pyridine, pyridazine, pyrimidine, pyrazine, or triazine; each of which is optionally substituted with one to three substituents independently selected from oxo, —NR₁₁R₁₁, —OR₁₁ or C₁-C₆ alkyl optionally substituted with aryl.

In yet a further compound of Formula (I), (II) or (III), or pharmaceutically acceptable salts thereof, R′ is hydrogen, halogen, C₁-C₃ alkyl, or C₃-C₆ cycloalkyl, preferably R′ is hydrogen, halogen, or methyl.

In yet a further compound of Formula (I), (II) or (III), or pharmaceutically acceptable salts thereof, R′ is hydrogen, —Cl, —F—, —Br, methyl, difluoromethyl, trifluoromethyl, methoxy, —NO₂, —CN, or a group of the formula:

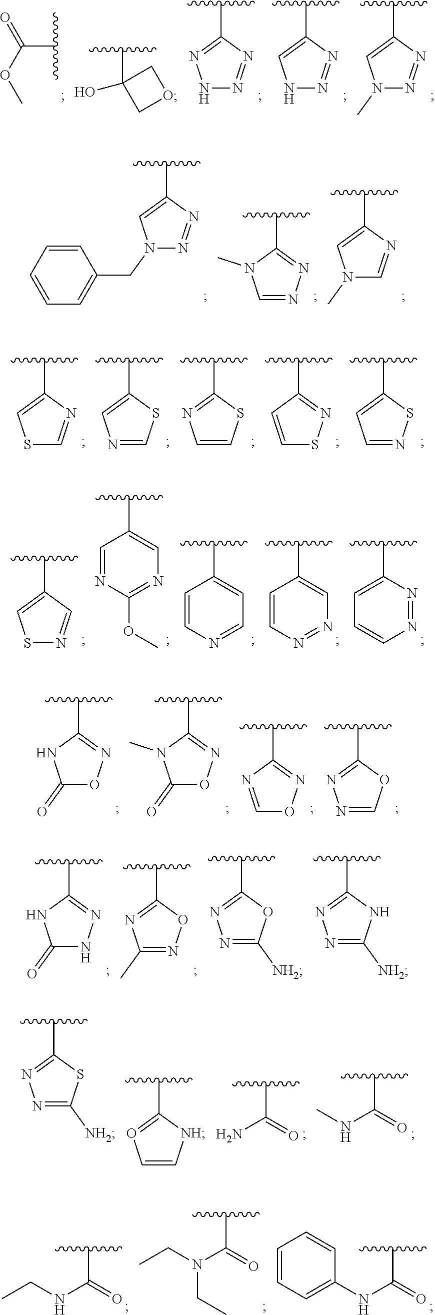

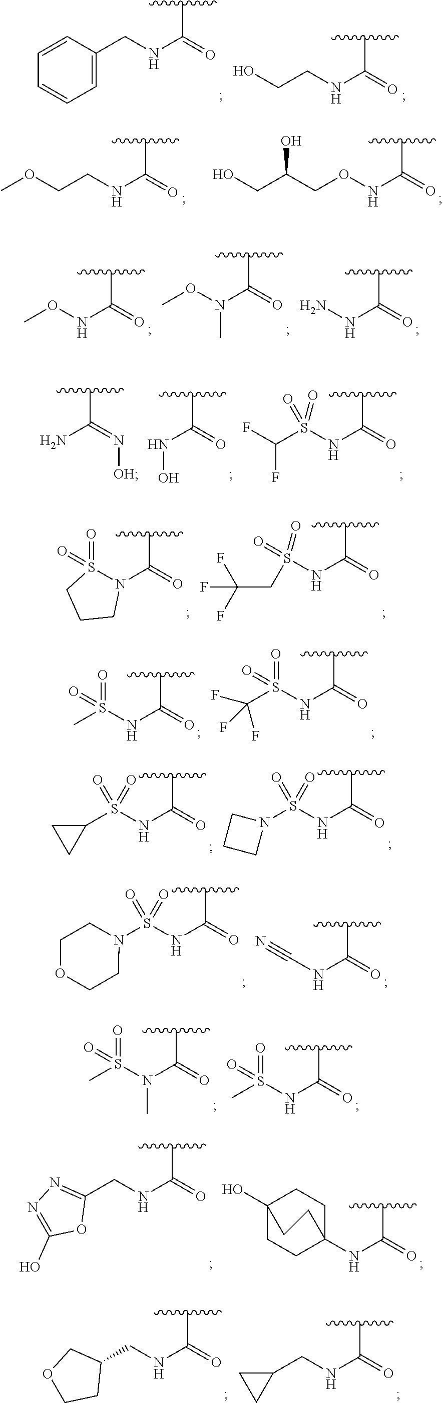

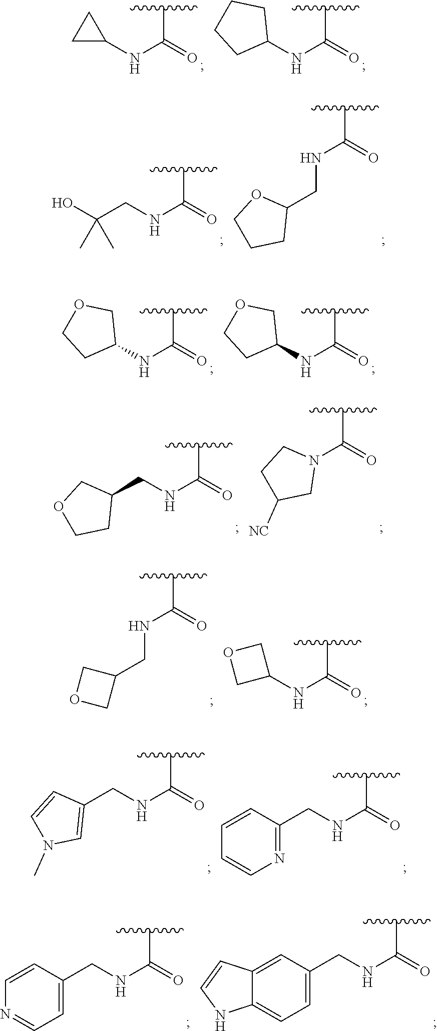

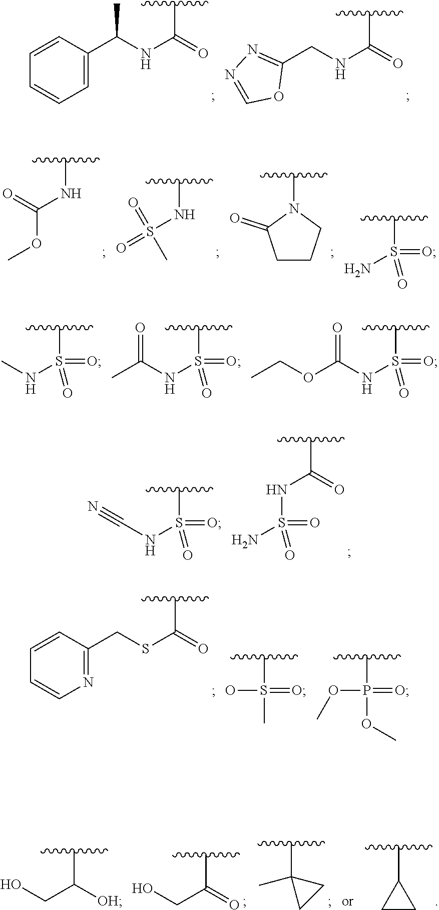

In yet a further compound of Formula (I), (II) or (III), or pharmaceutically acceptable salts thereof, R₁ is a group of the formula

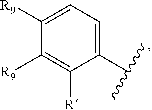

wherein each R₉ is independently —H, halogen, C₁-C₃ alkyl, or C₃-C₅ cycloalkyl, and R′ is hydrogen, halogen, C₁-C₃ alkyl, or C₃-C₆ cycloalkyl, preferably each R₉ is independently —H, halogen, methyl, or cyclopropyl and R′ is hydrogen, halogen, or C₁-C₃ alkyl.

In yet a further compound of Formula (I), (II) or (III), or pharmaceutically acceptable salts thereof, R₁ is a group of the formula

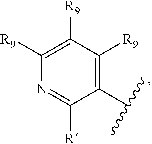

wherein each R₉ is independently —H, halogen, C₁-C₃ alkyl, or C₃-C₅ cycloalkyl, and R′ is hydrogen, halogen, C₁-C₃ alkyl, or C₃-C₆ cycloalkyl, preferably each R₉ is independently —H, halogen, methyl, or cyclopropyl and R′ is hydrogen, halogen, or C₁-C₃ alkyl.

In yet a further compound of Formula (I), (II) or (III), or pharmaceutically acceptable salts thereof, R₁ is a group of the formula:

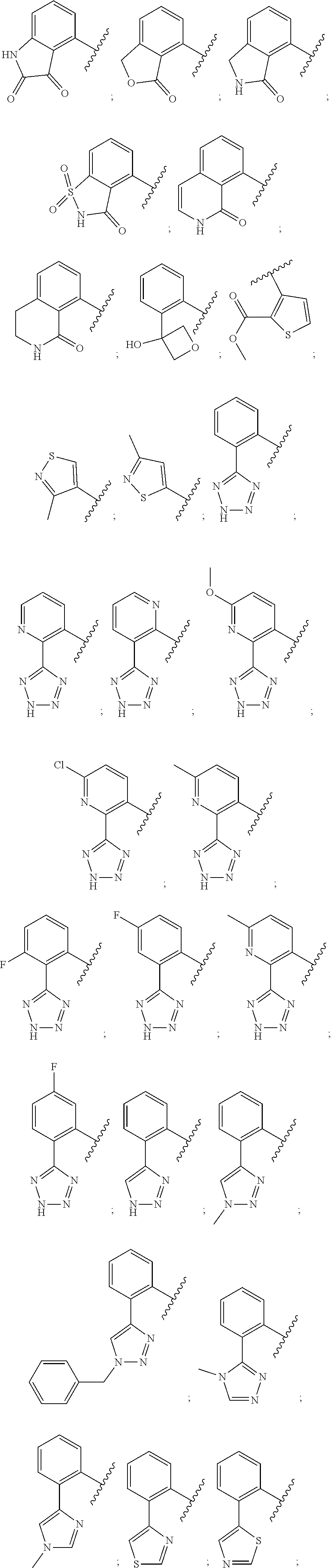

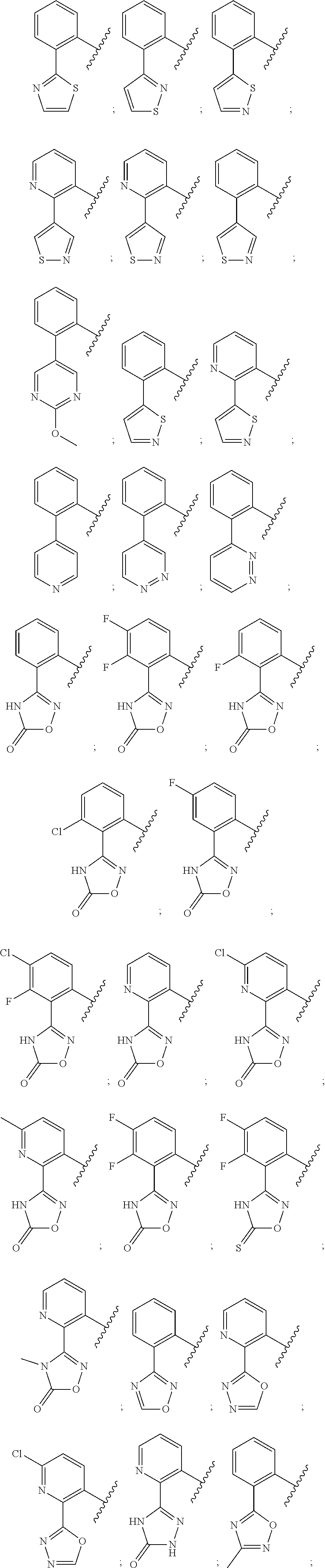

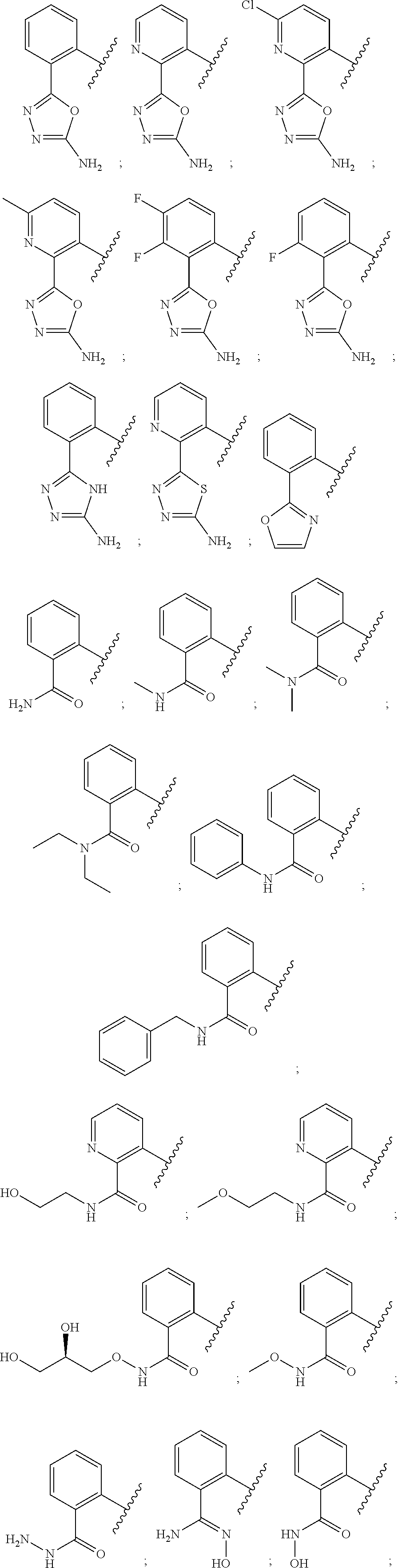

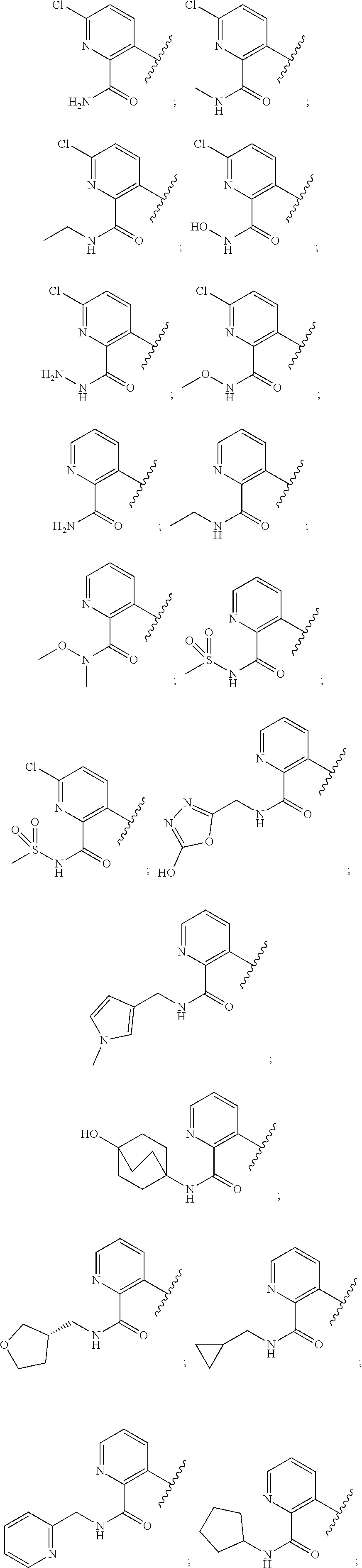

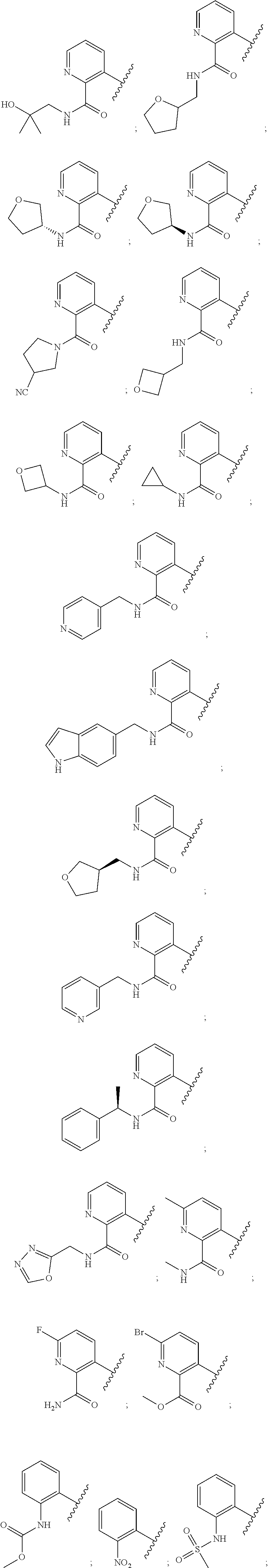

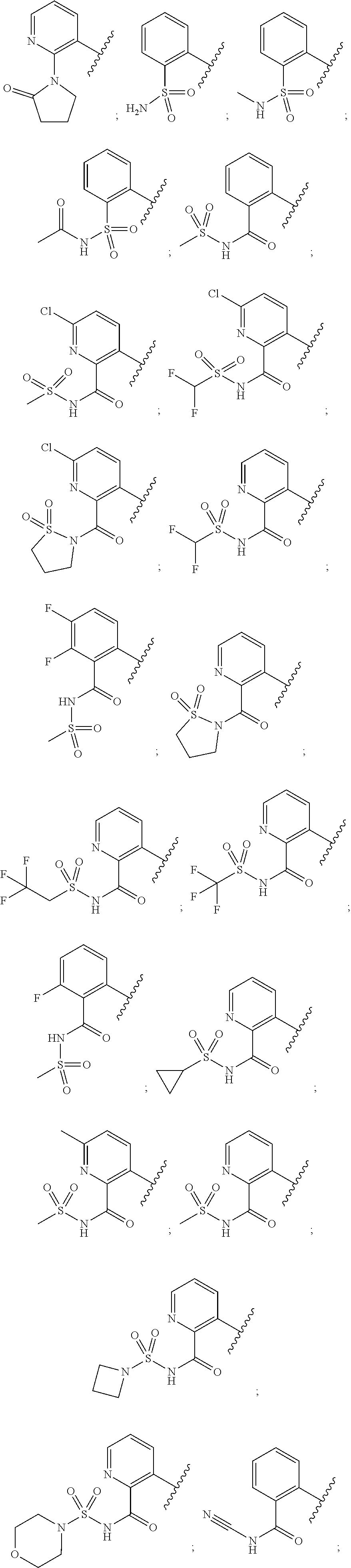

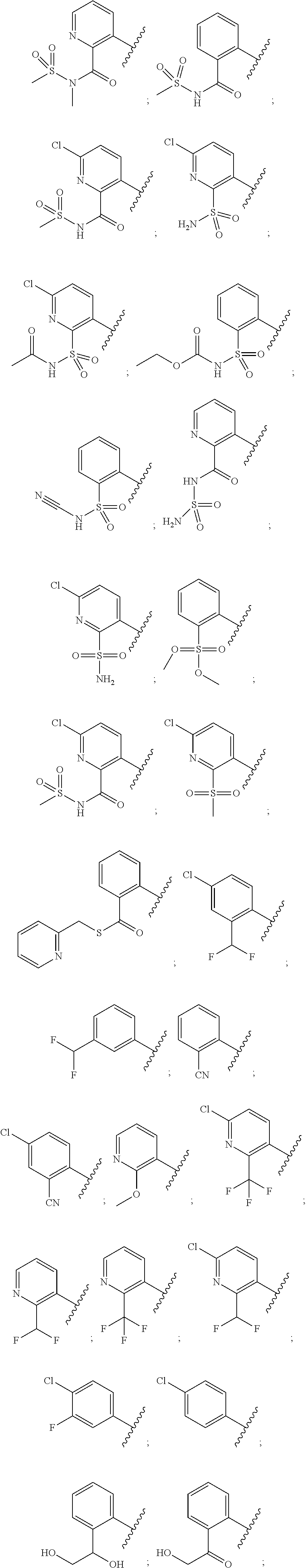

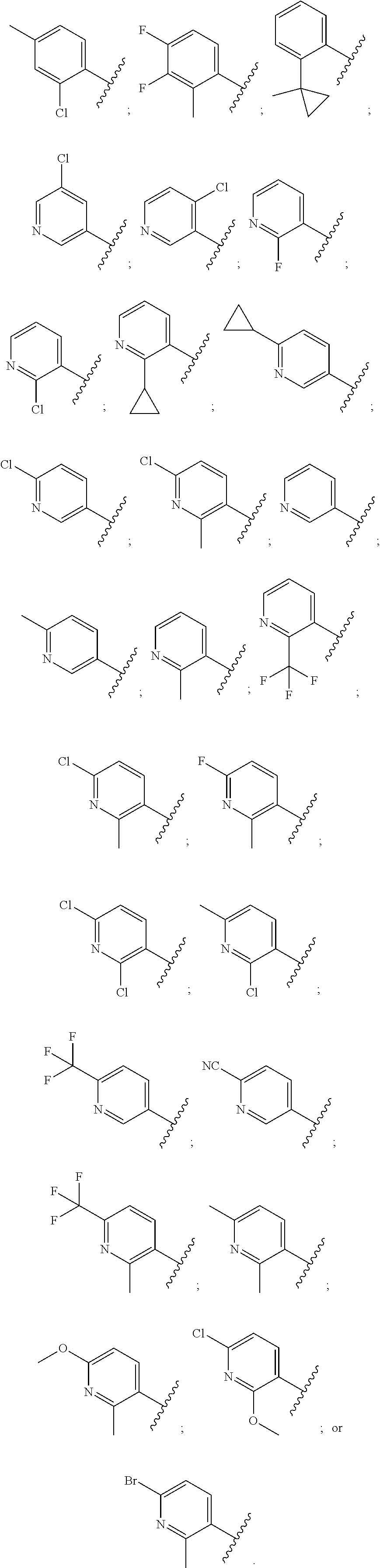

In yet a further compound of Formula (I), (II) or (III), or pharmaceutically acceptable salts thereof, R₁ is a group of the formula

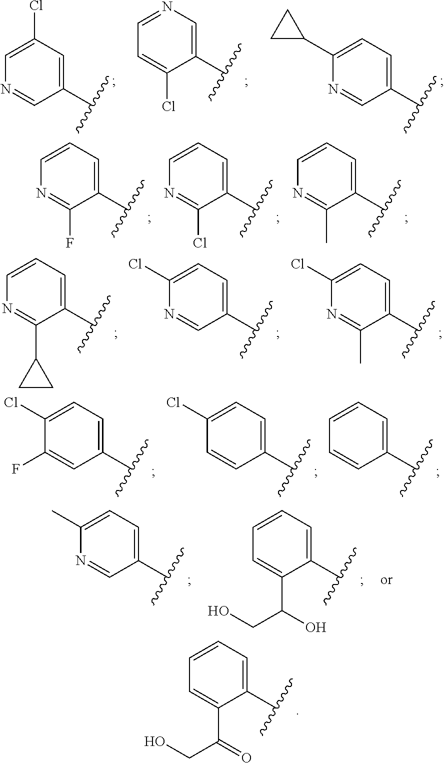

In yet a further compound of Formula (I), (II) or (III), or pharmaceutically acceptable salts thereof, R₂ is a group of the formula

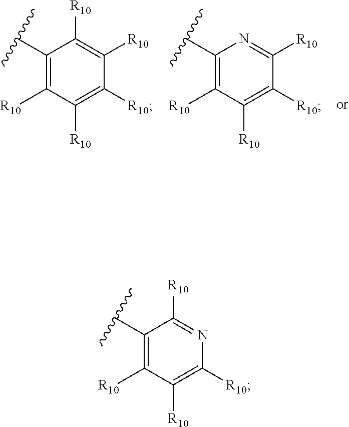

wherein each R₁₀ is independently —H, —CN, halogen, C₁-C₆ alkyl, C₁-C₆ haloalkyl, or —CO₂C₁-C₃ alkyl; or R₂ is an optionally substituted pyrazole or an optionally substituted indazole, each of which is optionally substituted with one to three substituents each independently selected from —CN, halogen, C₁-C₆ alkyl, C₁-C₆ haloalkyl, or —CO₂C₁-C₃ alkyl. Preferably R₂ is a group of the formula

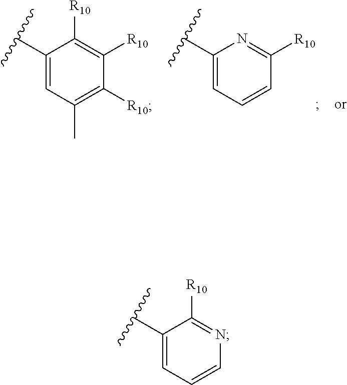

wherein each R₁₀ is independently —H, —CN, or halogen; or R₂ is an optionally substituted pyrazole or an optionally substituted indazole, each of which is optionally substituted with a C₁-C₃ alkyl.

In yet a further compound of Formula (I), (II) or (III), or pharmaceutically acceptable salts thereof, R₂ is a group of the formula:

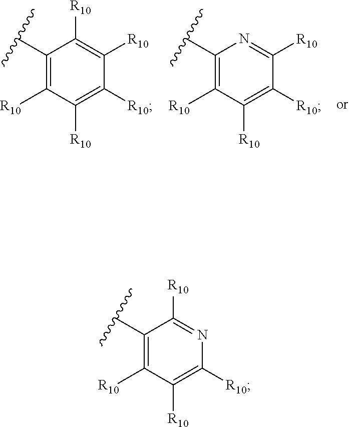

wherein each R₁₀ is independently —H, —CN, C₁-C₆ haloalkyl, —NR₁₁R₁₁, —CH₂—NR₁₁R₁₁, —CO₂C₁-C₃ alkyl or —CH₂—CO₂C₁-C₃ alkyl; or R₂ is an optionally substituted indazole, which is optionally substituted with one to three substituents each independently selected from —CN, halogen, C₁-C₆ alkyl, or C₁-C₆ haloalkyl.

In yet a further compound of Formula (I), (II) or (III), or pharmaceutically acceptable salts thereof, R₂ is a group of the formula

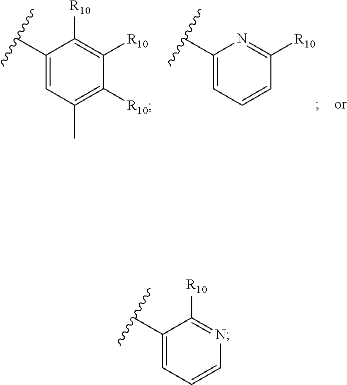

wherein each R₁₀ is independently —H, —CN, or C₁-C₃ haloalkyl; or R₂ is an optionally substituted indazole, which is optionally substituted with a C₁-C₃ alkyl.

In yet a further compound of Formula (I), (II) or (III), or pharmaceutically acceptable salts thereof, R₂ is a group of the formula:

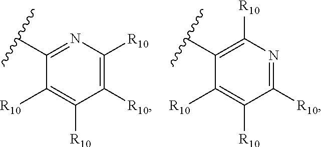

wherein R₁₀ is selected from —H, —CN, halogen, C₁-C₆ haloalkyl, C₁-C₆ alkoxy, and C₁-C₆ haloalkoxy.

In yet a further compound of Formula (I), (II) or (III), or pharmaceutically acceptable salts thereof, R₂ is a group of the formula:

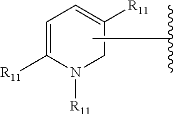

wherein R₁₁ is selected from hydrogen, alkyl, cycloalkyl, and haloalkyl.

In yet a further compound of Formula (I), (II) or (III), or pharmaceutically acceptable salts thereof, R₂ is pyrazole optionally substituted with alkyl or triazole optionally substituted with alkyl.

In yet a further compound of Formula (I), (II) or (III), or pharmaceutically acceptable salts thereof, R₂ is a group of the formula:

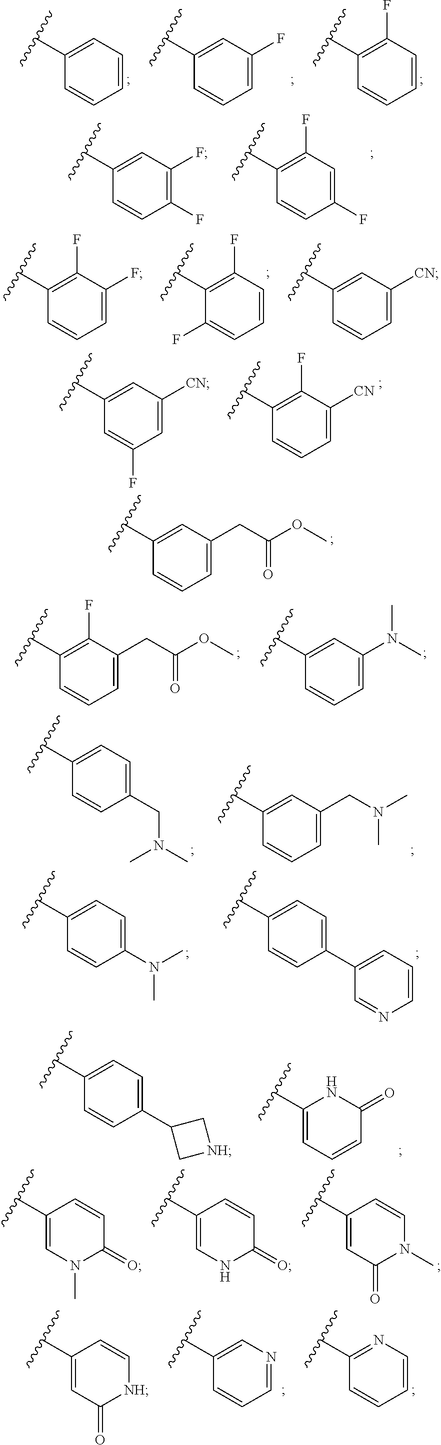

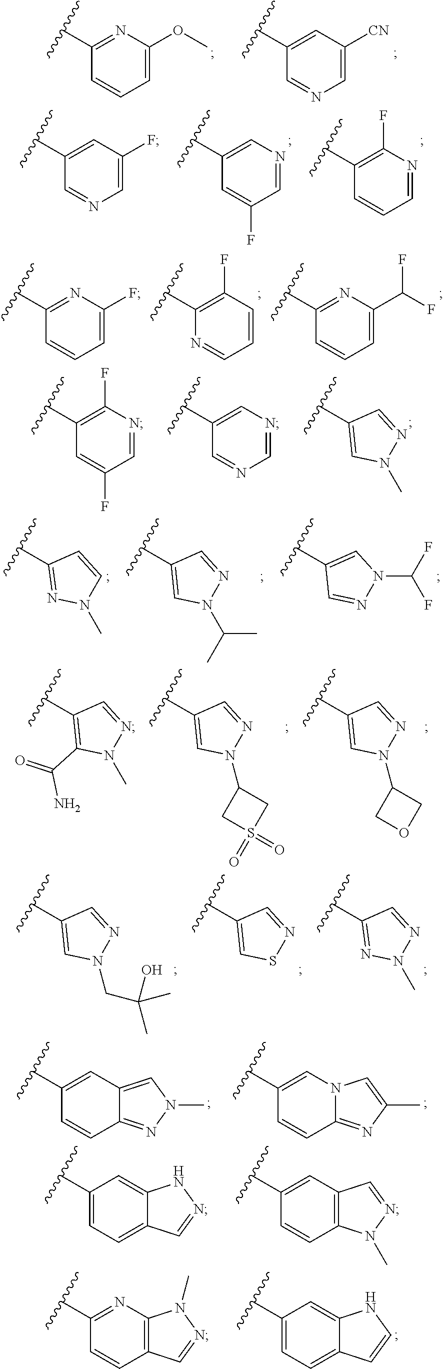

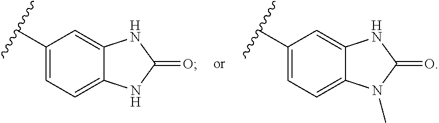 [SMILES: *c1ccc2[nH]c(=O)[nH]c2c1 ; *c1ccc2c(c1)[nH]c(=O)n2C]

In yet a further compound of Formula (I), (II) or (III), or pharmaceutically acceptable salts thereof, R₂ is a group of the formula

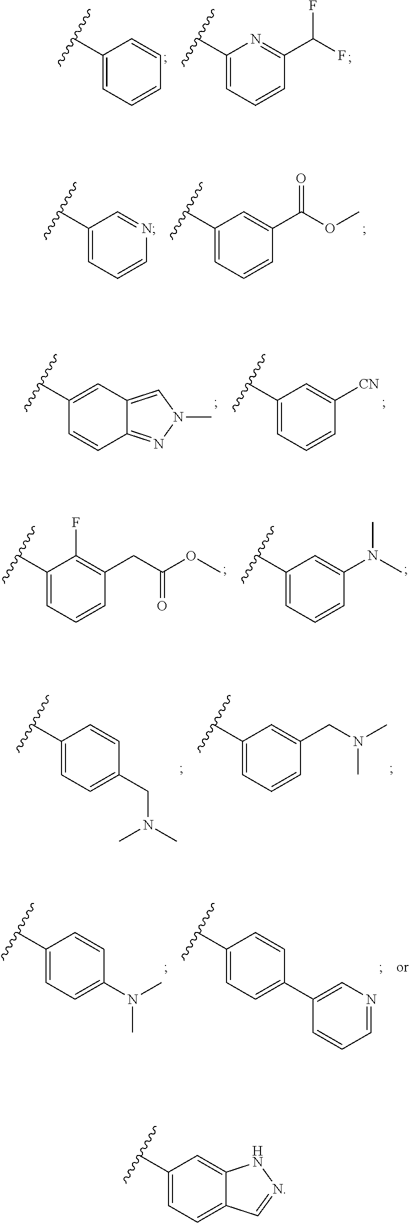

In yet a further compound of Formula (I), the compound is selected from:

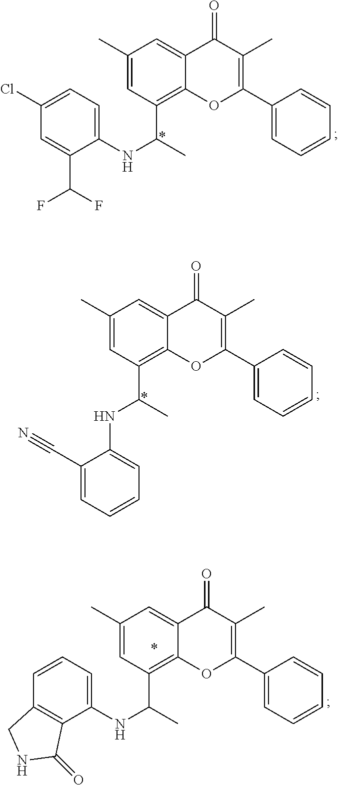

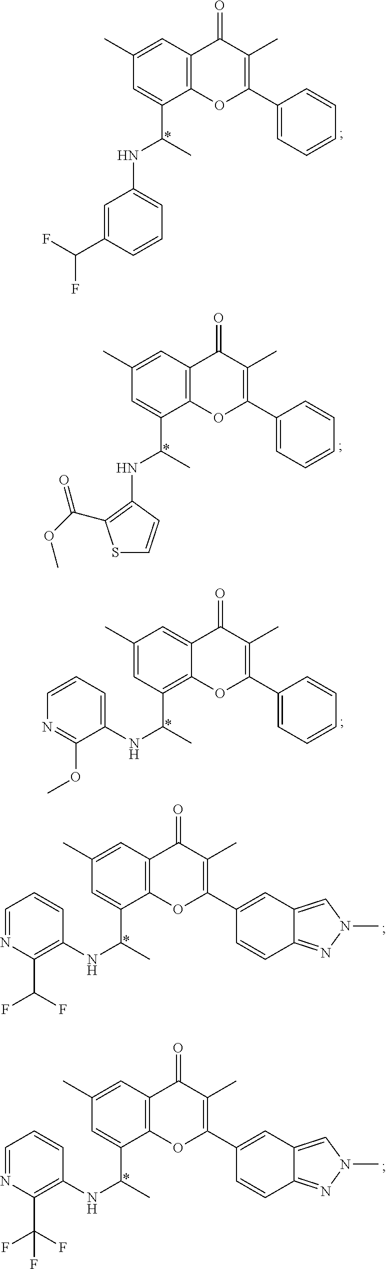

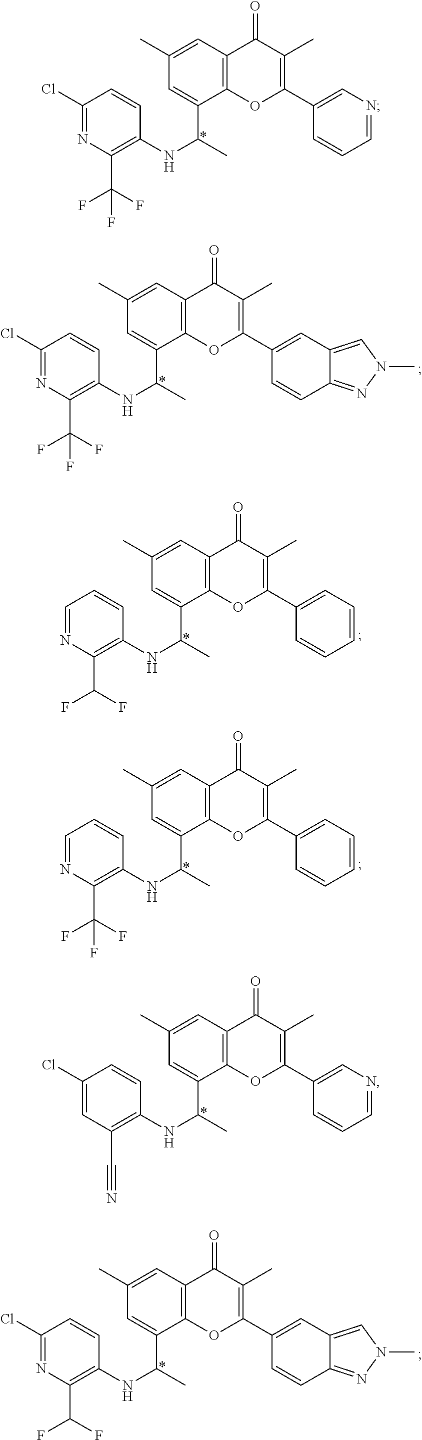 [SMILES: Cc1cc(C(C)Nc2ccc(Cl)nc2C(F)(F)F)c2oc(-c3cccnc3)c(C)c(=O)c2c1 ; Cc1cc(C(C)Nc2ccc(Cl)nc2C(F)(F)F)c2oc(-c3ccc4nn(C)cc4c3)c(C)c(=O)c2c1 ; Cc1cc(C(C)Nc2cccnc2C(F)F)c2oc(-c3ccccc3)c(C)c(=O)c2c1 ; Cc1cc(C(C)Nc2cccnc2C(F)(F)F)c2oc(-c3ccccc3)c(C)c(=O)c2c1 ; Cc1cc(C(C)Nc2ccc(Cl)cc2C#N)c2oc(-c3cccnc3)c(C)c(=O)c2c1 ; Cc1cc(C(C)Nc2ccc(Cl)nc2C(F)F)c2oc(-c3ccc4nn(C)cc4c3)c(C)c(=O)c2c1]

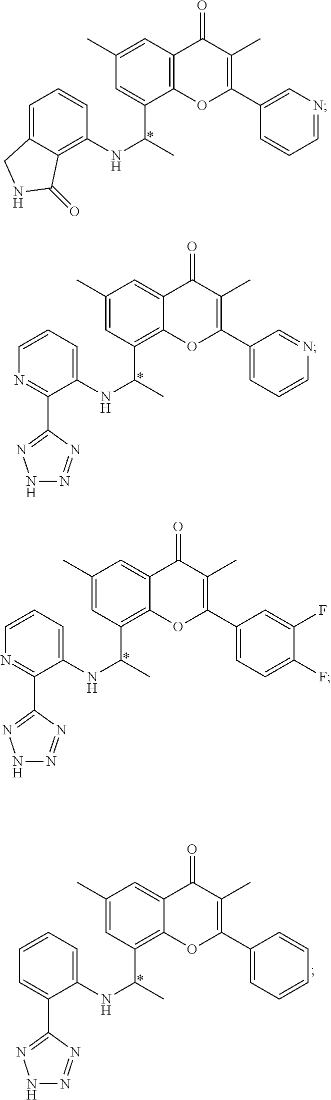 [SMILES: Cc1cc(C(C)Nc2cccc3c2C(=O)NC3)c2oc(-c3cccnc3)c(C)c(=O)c2c1 ; Cc1cc(C(C)Nc2cccnc2-c2nn[nH]n2)c2oc(-c3cccnc3)c(C)c(=O)c2c1 ; Cc1cc(C(C)Nc2cccnc2-c2nn[nH]n2)c2oc(-c3ccc(F)c(F)c3)c(C)c(=O)c2c1 ; Cc1cc(C(C)Nc2ccccc2-c2nn[nH]n2)c2oc(-c3ccccc3)c(C)c(=O)c2c1]

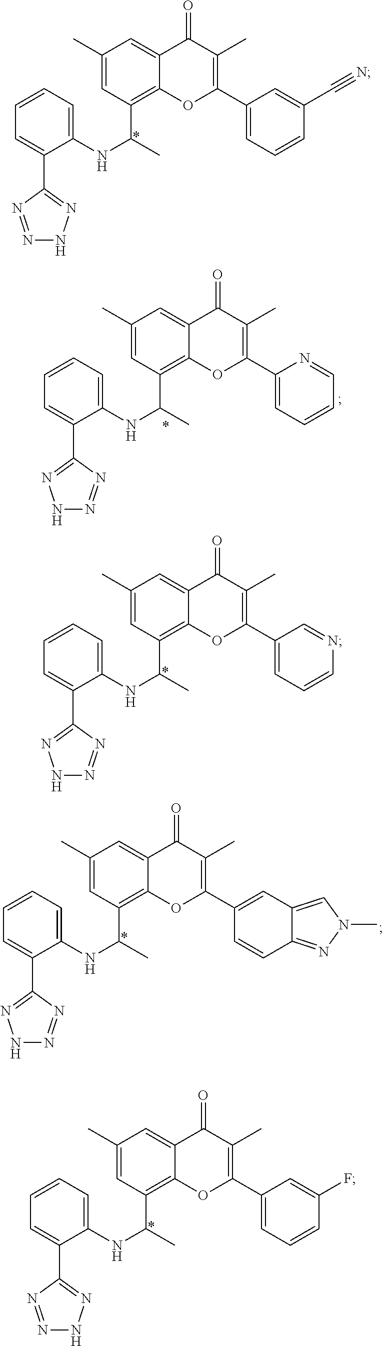 [SMILES: Cc1cc(C(C)Nc2ccccc2-c2nn[nH]n2)c2oc(-c3cccc(C#N)c3)c(C)c(=O)c2c1 ; Cc1cc(C(C)Nc2ccccc2-c2nn[nH]n2)c2oc(-c3ccccn3)c(C)c(=O)c2c1 ; Cc1cc(C(C)Nc2ccccc2-c2nn[nH]n2)c2oc(-c3cccnc3)c(C)c(=O)c2c1 ; Cc1cc(C(C)Nc2ccccc2-c2nn[nH]n2)c2oc(-c3ccc4nn(C)cc4c3)c(C)c(=O)c2c1 ; Cc1cc(C(C)Nc2ccccc2-c2nn[nH]n2)c2oc(-c3cccc(F)c3)c(C)c(=O)c2c1]

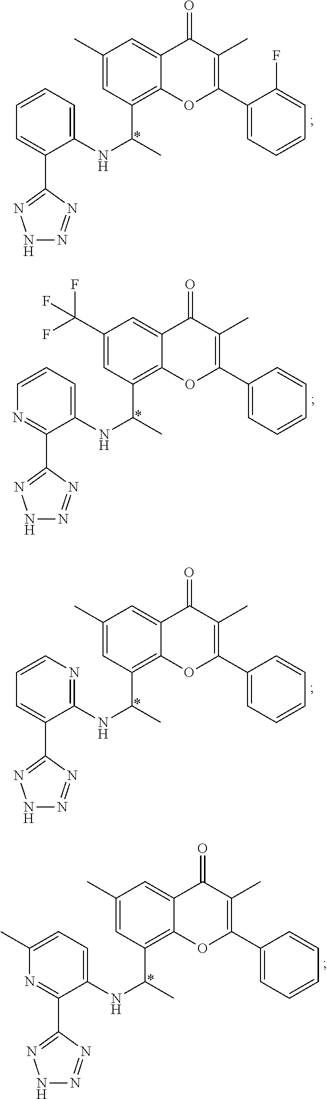

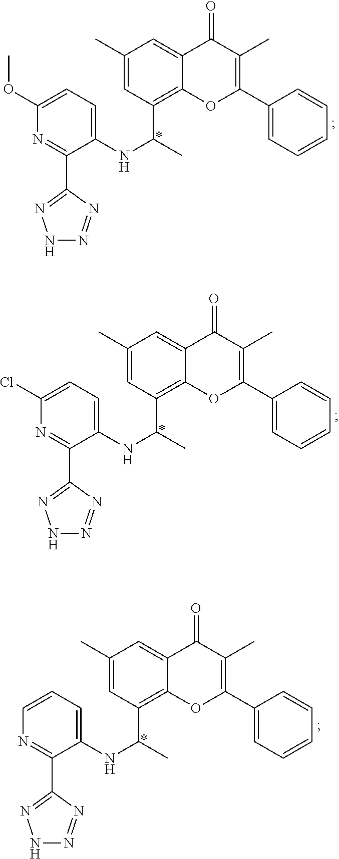 [SMILES: COc1ccc(NC(C)c2cc(C)cc3c(=O)c(C)c(-c4ccccc4)oc23)c(-c2nn[nH]n2)n1 ; Cc1cc(C(C)Nc2ccc(Cl)nc2-c2nn[nH]n2)c2oc(-c3ccccc3)c(C)c(=O)c2c1 ; Cc1cc(C(C)Nc2cccnc2-c2nn[nH]n2)c2oc(-c3ccccc3)c(C)c(=O)c2c1]

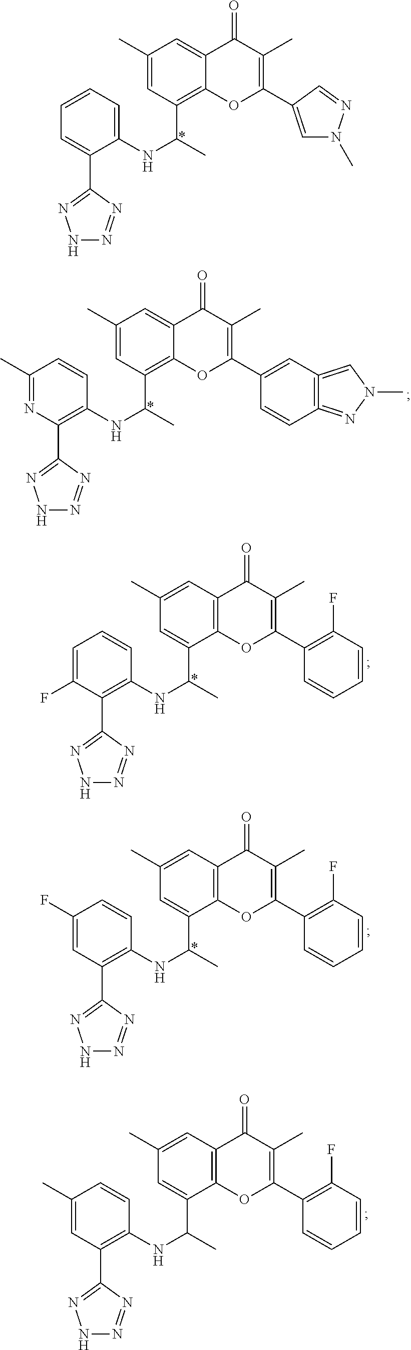

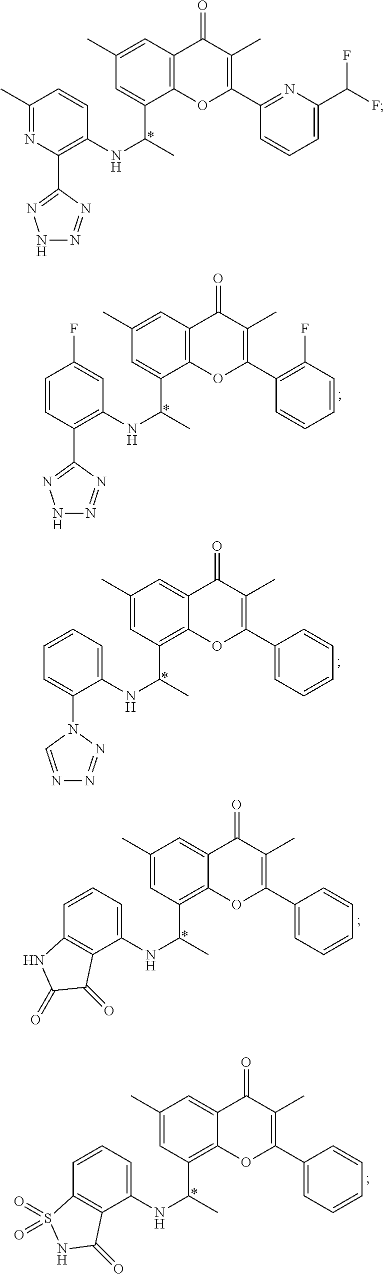 [SMILES: Cc1cc(C(C)Nc2ccc(C)nc2-c2nn[nH]n2)c2oc(-c3cccc(C(F)F)n3)c(C)c(=O)c2c1 ; Cc1cc(C(C)Nc2cc(F)ccc2-c2nn[nH]n2)c2oc(-c3ccccc3F)c(C)c(=O)c2c1 ; Cc1cc(C(C)Nc2ccccc2-n2cnnn2)c2oc(-c3ccccc3)c(C)c(=O)c2c1 ; Cc1cc(C(C)Nc2cccc3c2C(=O)C(=O)N3)c2oc(-c3ccccc3)c(C)c(=O)c2c1 ; Cc1cc(C(C)Nc2cccc3c2C(=O)NS3(=O)=O)c2oc(-c3ccccc3)c(C)c(=O)c2c1]

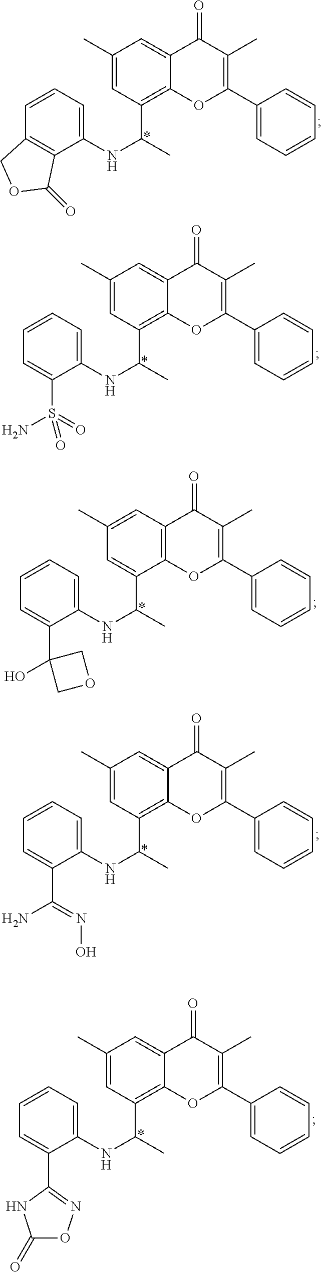 [SMILES: Cc1cc(C(C)Nc2cccc3c2C(=O)OC3)c2oc(-c3ccccc3)c(C)c(=O)c2c1 ; Cc1cc(C(C)Nc2ccccc2S(N)(=O)=O)c2oc(-c3ccccc3)c(C)c(=O)c2c1 ; Cc1cc(C(C)Nc2ccccc2C2(O)COC2)c2oc(-c3ccccc3)c(C)c(=O)c2c1 ; Cc1cc(C(C)Nc2ccccc2/C(N)=N/O)c2oc(-c3ccccc3)c(C)c(=O)c2c1 ; Cc1cc(C(C)Nc2ccccc2-c2noc(=O)[nH]2)c2oc(-c3ccccc3)c(C)c(=O)c2c1]

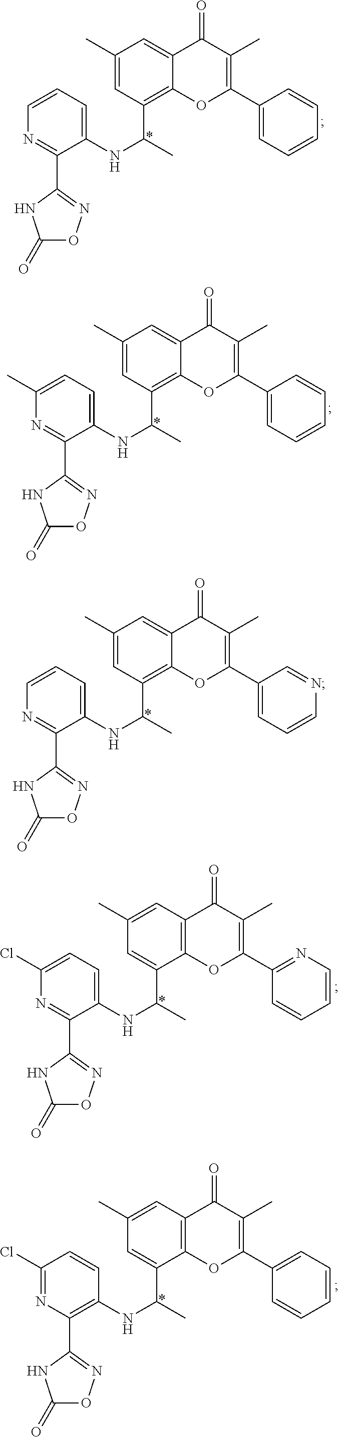 [SMILES: Cc1cc(C(C)Nc2cccnc2-c2noc(=O)[nH]2)c2oc(-c3ccccc3)c(C)c(=O)c2c1 ; Cc1cc(C(C)Nc2ccc(C)nc2-c2noc(=O)[nH]2)c2oc(-c3ccccc3)c(C)c(=O)c2c1 ; Cc1cc(C(C)Nc2cccnc2-c2noc(=O)[nH]2)c2oc(-c3cccnc3)c(C)c(=O)c2c1 ; Cc1cc(C(C)Nc2ccc(Cl)nc2-c2noc(=O)[nH]2)c2oc(-c3ccccn3)c(C)c(=O)c2c1 ; Cc1cc(C(C)Nc2ccc(Cl)nc2-c2noc(=O)[nH]2)c2oc(-c3ccccc3)c(C)c(=O)c2c1]

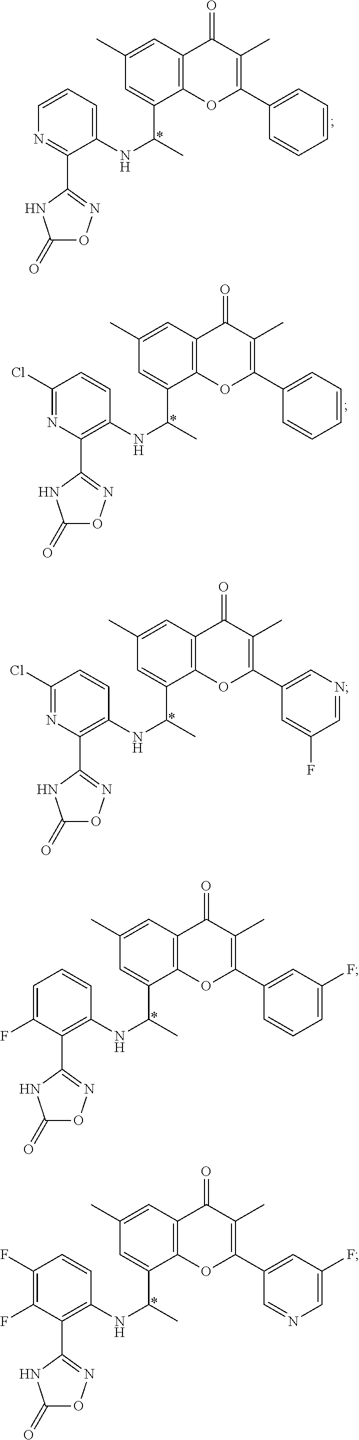 [SMILES: Cc1cc(C(C)Nc2cccnc2-c2noc(=O)[nH]2)c2oc(-c3ccccc3)c(C)c(=O)c2c1 ; Cc1cc(C(C)Nc2ccc(Cl)nc2-c2noc(=O)[nH]2)c2oc(-c3ccccc3)c(C)c(=O)c2c1 ; Cc1cc(C(C)Nc2ccc(Cl)nc2-c2noc(=O)[nH]2)c2oc(-c3cncc(F)c3)c(C)c(=O)c2c1 ; Cc1cc(C(C)Nc2cccc(F)c2-c2noc(=O)[nH]2)c2oc(-c3cccc(F)c3)c(C)c(=O)c2c1 ; Cc1cc(C(C)Nc2ccc(F)c(F)c2-c2noc(=O)[nH]2)c2oc(-c3cncc(F)c3)c(C)c(=O)c2c1]

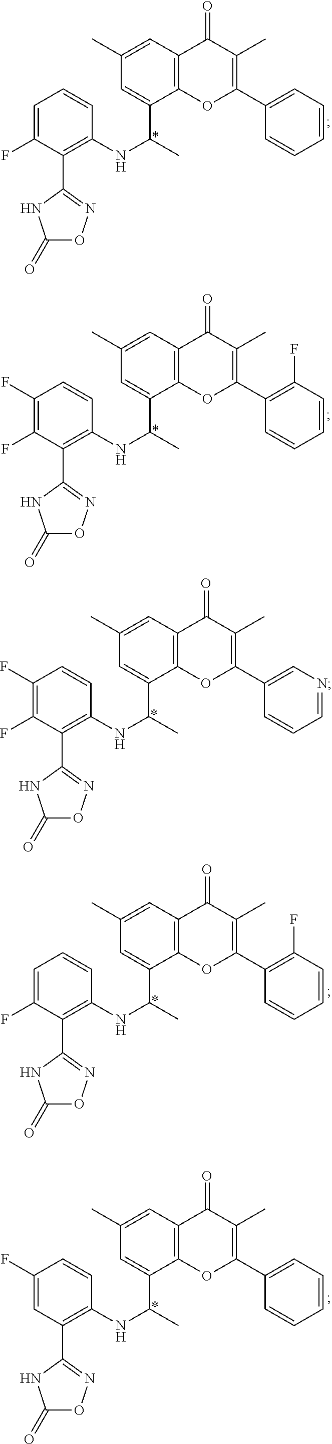 [SMILES: Cc1cc(C(C)Nc2cccc(F)c2-c2noc(=O)[nH]2)c2oc(-c3ccccc3)c(C)c(=O)c2c1 ; Cc1cc(C(C)Nc2ccc(F)c(F)c2-c2noc(=O)[nH]2)c2oc(-c3ccccc3F)c(C)c(=O)c2c1 ; Cc1cc(C(C)Nc2ccc(F)c(F)c2-c2noc(=O)[nH]2)c2oc(-c3cccnc3)c(C)c(=O)c2c1 ; Cc1cc(C(C)Nc2cccc(F)c2-c2noc(=O)[nH]2)c2oc(-c3ccccc3F)c(C)c(=O)c2c1 ; Cc1cc(C(C)Nc2ccc(F)cc2-c2noc(=O)[nH]2)c2oc(-c3ccccc3)c(C)c(=O)c2c1]

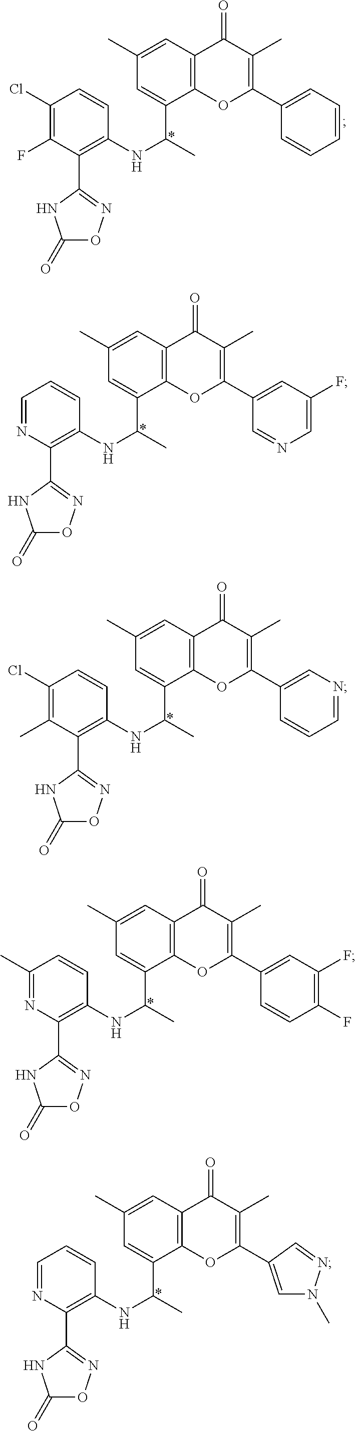 [SMILES: Cc1cc(C(C)Nc2ccc(Cl)c(F)c2-c2noc(=O)[nH]2)c2oc(-c3ccccc3)c(C)c(=O)c2c1 ; Cc1cc(C(C)Nc2cccnc2-c2noc(=O)[nH]2)c2oc(-c3cncc(F)c3)c(C)c(=O)c2c1 ; Cc1cc(C(C)Nc2ccc(Cl)c(C)c2-c2noc(=O)[nH]2)c2oc(-c3cccnc3)c(C)c(=O)c2c1 ; Cc1cc(C(C)Nc2ccc(C)nc2-c2noc(=O)[nH]2)c2oc(-c3ccc(F)c(F)c3)c(C)c(=O)c2c1 ; Cc1cc(C(C)Nc2cccnc2-c2noc(=O)[nH]2)c2oc(-c3cnn(C)c3)c(C)c(=O)c2c1]

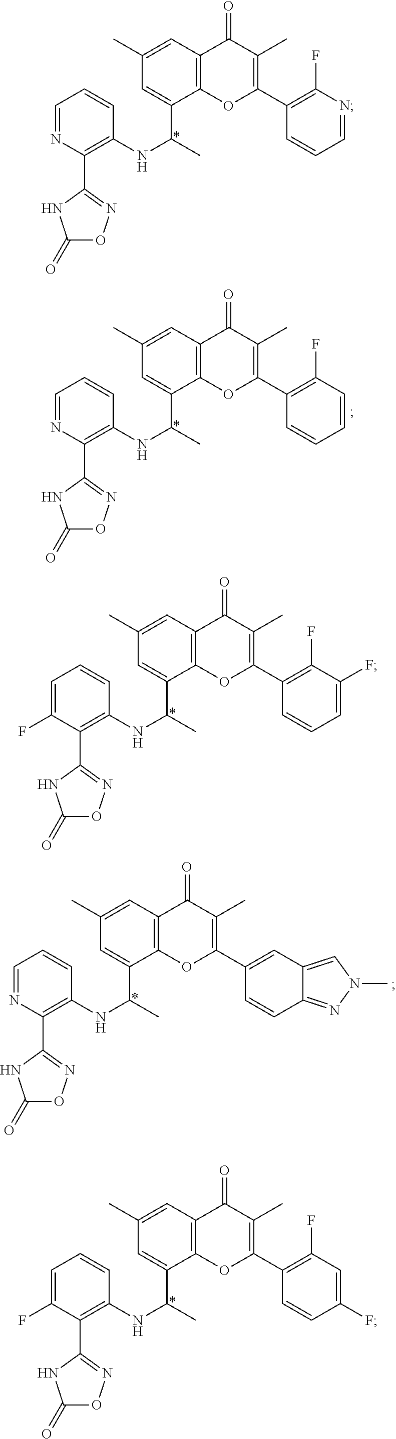 [SMILES: Cc1cc(C(C)Nc2cccnc2-c2noc(=O)[nH]2)c2oc(-c3cccnc3F)c(C)c(=O)c2c1 ; Cc1cc(C(C)Nc2cccnc2-c2noc(=O)[nH]2)c2oc(-c3ccccc3F)c(C)c(=O)c2c1 ; Cc1cc(C(C)Nc2cccc(F)c2-c2noc(=O)[nH]2)c2oc(-c3cccc(F)c3F)c(C)c(=O)c2c1 ; Cc1cc(C(C)Nc2cccnc2-c2noc(=O)[nH]2)c2oc(-c3ccc4nn(C)cc4c3)c(C)c(=O)c2c1 ; Cc1cc(C(C)Nc2cccc(F)c2-c2noc(=O)[nH]2)c2oc(-c3ccc(F)cc3F)c(C)c(=O)c2c1]

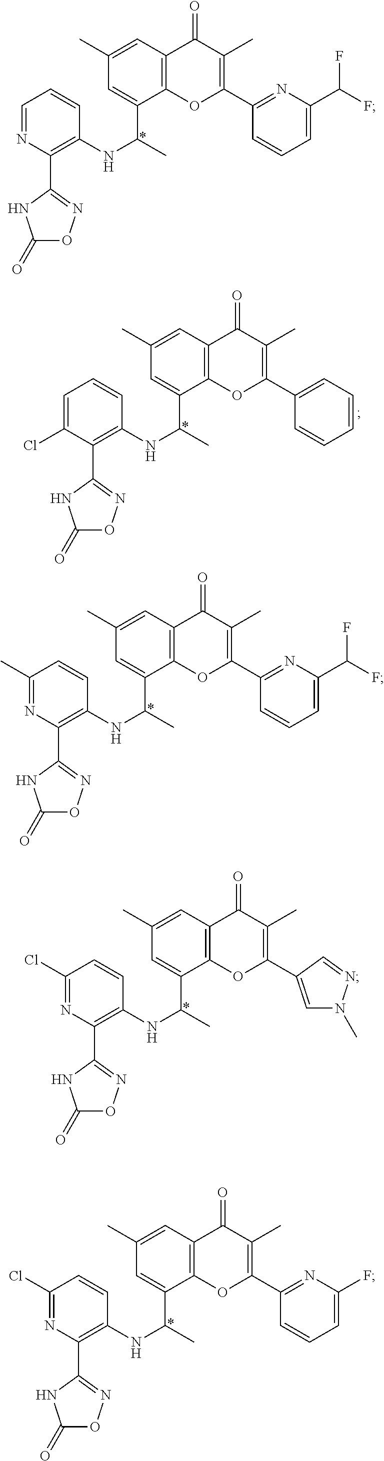 [SMILES: Cc1cc(C(C)Nc2cccnc2-c2noc(=O)[nH]2)c2oc(-c3cccc(C(F)F)n3)c(C)c(=O)c2c1 ; Cc1cc(C(C)Nc2cccc(Cl)c2-c2noc(=O)[nH]2)c2oc(-c3ccccc3)c(C)c(=O)c2c1 ; Cc1cc(C(C)Nc2ccc(C)nc2-c2noc(=O)[nH]2)c2oc(-c3cccc(C(F)F)n3)c(C)c(=O)c2c1 ; Cc1cc(C(C)Nc2ccc(Cl)nc2-c2noc(=O)[nH]2)c2oc(-c3cnn(C)c3)c(C)c(=O)c2c1 ; Cc1cc(C(C)Nc2ccc(Cl)nc2-c2noc(=O)[nH]2)c2oc(-c3cccc(F)n3)c(C)c(=O)c2c1]

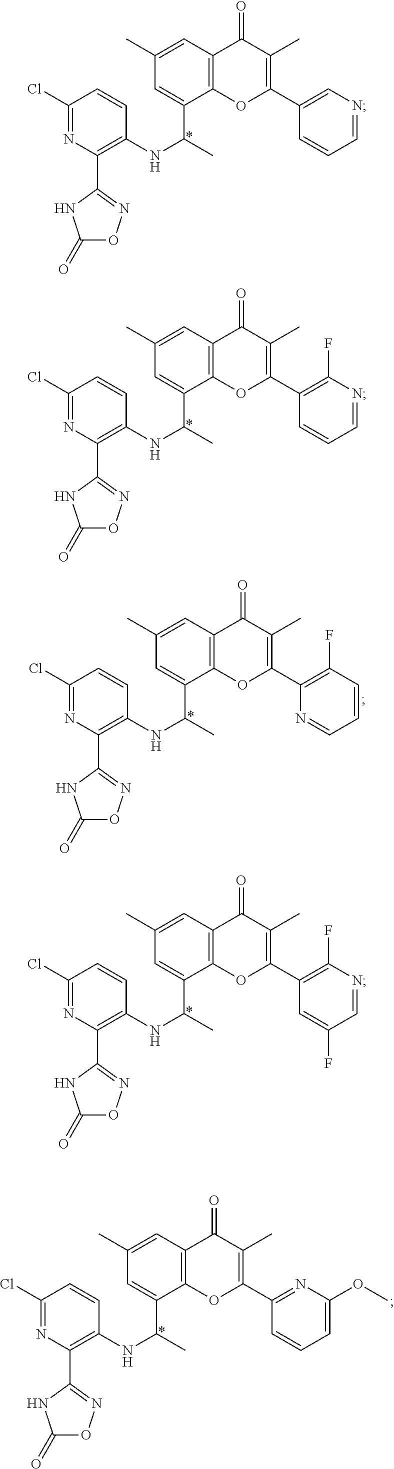 [SMILES: Cc1cc(C(C)Nc2ccc(Cl)nc2-c2noc(=O)[nH]2)c2oc(-c3cccnc3)c(C)c(=O)c2c1 ; Cc1cc(C(C)Nc2ccc(Cl)nc2-c2noc(=O)[nH]2)c2oc(-c3cccnc3F)c(C)c(=O)c2c1 ; Cc1cc(C(C)Nc2ccc(Cl)nc2-c2noc(=O)[nH]2)c2oc(-c3ncccc3F)c(C)c(=O)c2c1 ; Cc1cc(C(C)Nc2ccc(Cl)nc2-c2noc(=O)[nH]2)c2oc(-c3cc(F)cnc3F)c(C)c(=O)c2c1 ; COc1cccc(-c2oc3c(C(C)Nc4ccc(Cl)nc4-c4noc(=O)[nH]4)cc(C)cc3c(=O)c2C)n1]

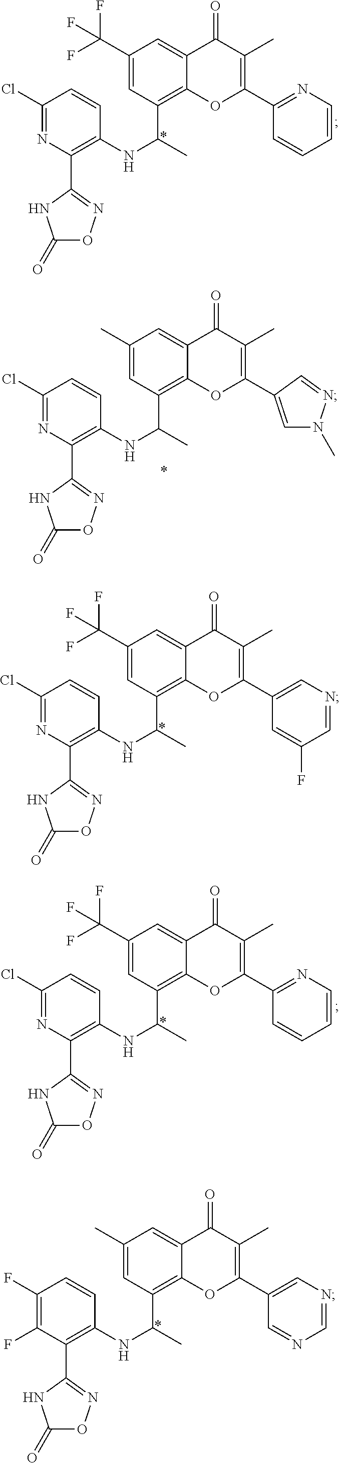 [SMILES: Cc1c(-c2ccccn2)oc2c(C(C)Nc3ccc(Cl)nc3-c3noc(=O)[nH]3)cc(C(F)(F)F)cc2c1=O ; Cc1cc(C(C)Nc2ccc(Cl)nc2-c2noc(=O)[nH]2)c2oc(-c3cnn(C)c3)c(C)c(=O)c2c1 ; Cc1c(-c2cncc(F)c2)oc2c(C(C)Nc3ccc(Cl)nc3-c3noc(=O)[nH]3)cc(C(F)(F)F)cc2c1=O ; Cc1c(-c2ccccn2)oc2c(C(C)Nc3ccc(Cl)nc3-c3noc(=O)[nH]3)cc(C(F)(F)F)cc2c1=O ; Cc1cc(C(C)Nc2ccc(F)c(F)c2-c2noc(=O)[nH]2)c2oc(-c3cncnc3)c(C)c(=O)c2c1]

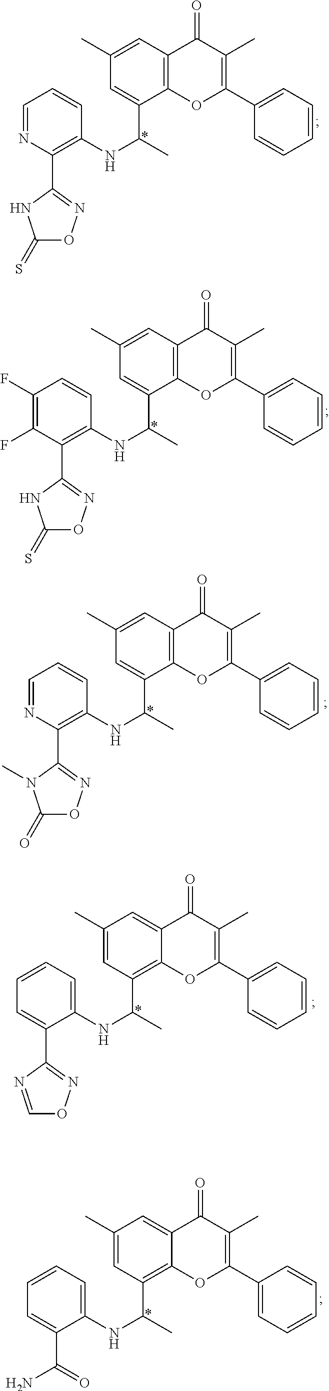 [SMILES: Cc1cc(C(C)Nc2cccnc2-c2noc(=S)[nH]2)c2oc(-c3ccccc3)c(C)c(=O)c2c1 ; Cc1cc(C(C)Nc2ccc(F)c(F)c2-c2noc(=S)[nH]2)c2oc(-c3ccccc3)c(C)c(=O)c2c1 ; Cc1cc(C(C)Nc2cccnc2-c2noc(=O)n2C)c2oc(-c3ccccc3)c(C)c(=O)c2c1 ; Cc1cc(C(C)Nc2ccccc2-c2ncon2)c2oc(-c3ccccc3)c(C)c(=O)c2c1 ; Cc1cc(C(C)Nc2ccccc2C(N)=O)c2oc(-c3ccccc3)c(C)c(=O)c2c1]

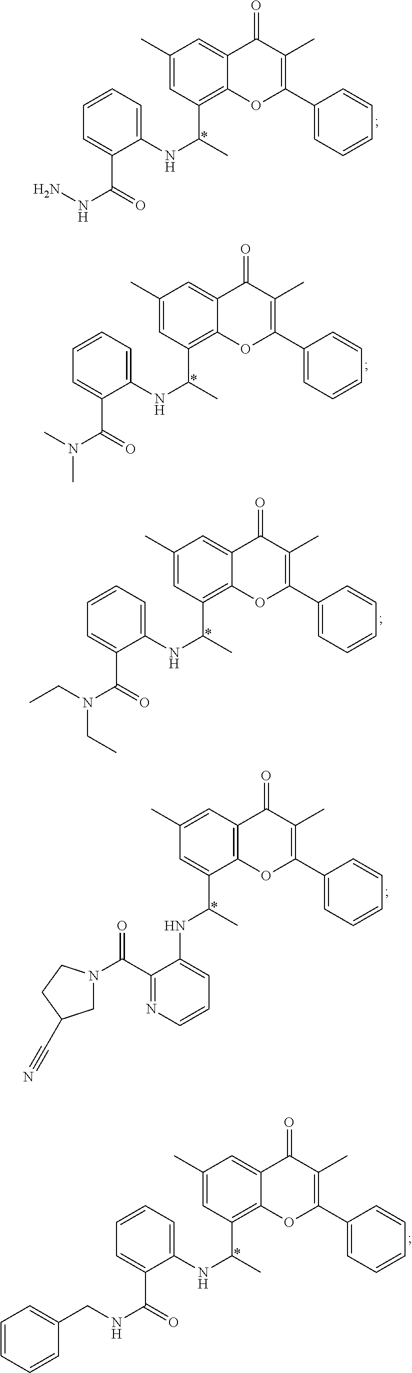 [SMILES: Cc1cc(C(C)Nc2ccccc2C(=O)NN)c2oc(-c3ccccc3)c(C)c(=O)c2c1 ; Cc1cc(C(C)Nc2ccccc2C(=O)N(C)C)c2oc(-c3ccccc3)c(C)c(=O)c2c1 ; CCN(CC)C(=O)c1ccccc1NC(C)c1cc(C)cc2c(=O)c(C)c(-c3ccccc3)oc12 ; Cc1cc(C(C)Nc2cccnc2C(=O)N2CCC(C#N)C2)c2oc(-c3ccccc3)c(C)c(=O)c2c1 ; Cc1cc(C(C)Nc2ccccc2C(=O)NCc2ccccc2)c2oc(-c3ccccc3)c(C)c(=O)c2c1]

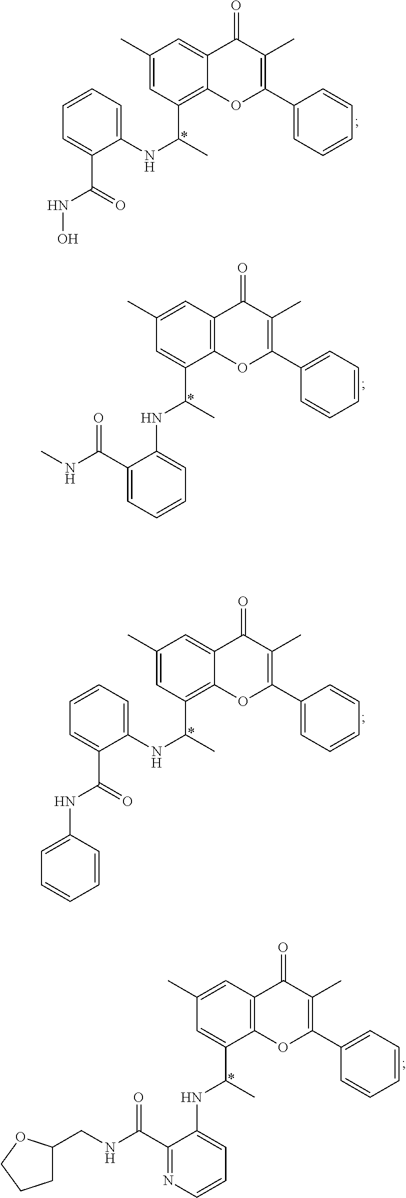 [SMILES: Cc1cc(C(C)Nc2ccccc2C(=O)NO)c2oc(-c3ccccc3)c(C)c(=O)c2c1 ; CNC(=O)c1ccccc1NC(C)c1cc(C)cc2c(=O)c(C)c(-c3ccccc3)oc12 ; Cc1cc(C(C)Nc2ccccc2C(=O)Nc2ccccc2)c2oc(-c3ccccc3)c(C)c(=O)c2c1 ; Cc1cc(C(C)Nc2cccnc2C(=O)NCC2CCCO2)c2oc(-c3ccccc3)c(C)c(=O)c2c1]

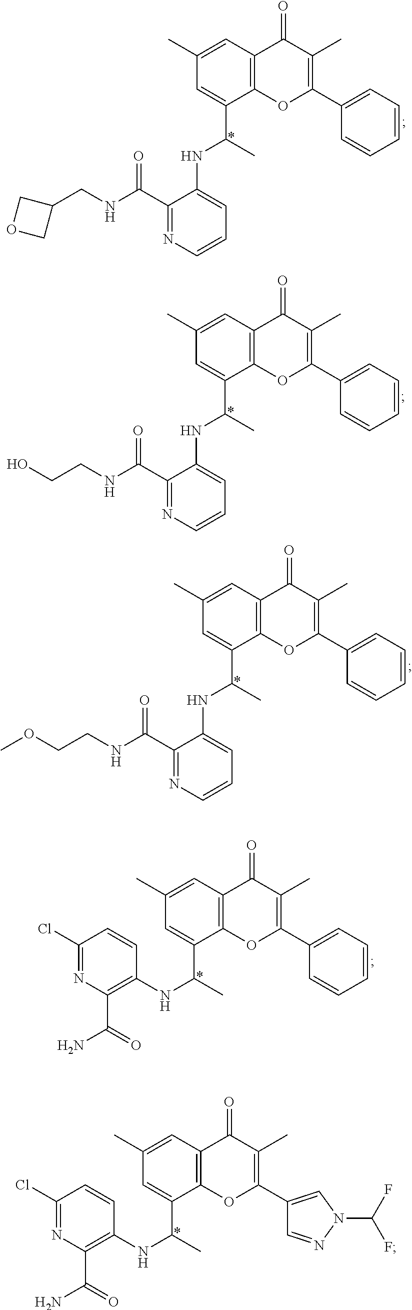 [SMILES: Cc1cc(C(C)Nc2cccnc2C(=O)NCC2COC2)c2oc(-c3ccccc3)c(C)c(=O)c2c1 ; Cc1cc(C(C)Nc2cccnc2C(=O)NCCO)c2oc(-c3ccccc3)c(C)c(=O)c2c1 ; COCCNC(=O)c1ncccc1NC(C)c1cc(C)cc2c(=O)c(C)c(-c3ccccc3)oc12 ; Cc1cc(C(C)Nc2ccc(Cl)nc2C(N)=O)c2oc(-c3ccccc3)c(C)c(=O)c2c1 ; Cc1cc(C(C)Nc2ccc(Cl)nc2C(N)=O)c2oc(-c3cnn(C(F)F)c3)c(C)c(=O)c2c1]

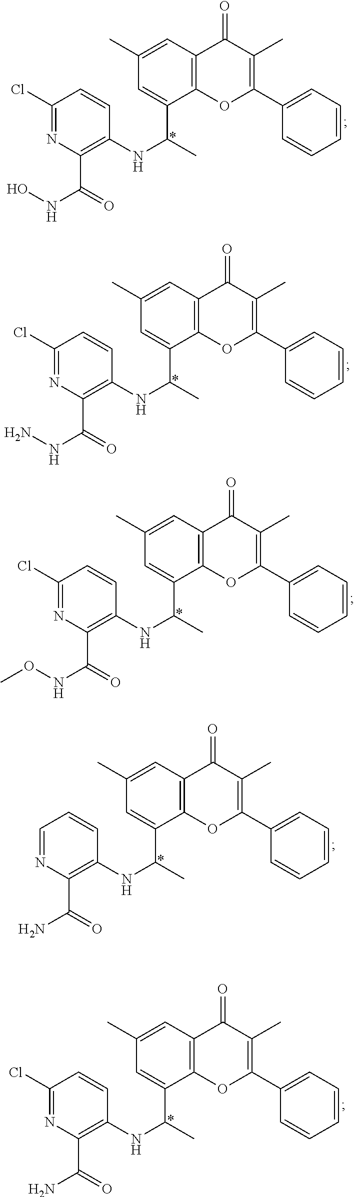 [SMILES: Cc1cc(C(C)Nc2ccc(Cl)nc2C(=O)NO)c2oc(-c3ccccc3)c(C)c(=O)c2c1 ; Cc1cc(C(C)Nc2ccc(Cl)nc2C(=O)NN)c2oc(-c3ccccc3)c(C)c(=O)c2c1 ; CONC(=O)c1nc(Cl)ccc1NC(C)c1cc(C)cc2c(=O)c(C)c(-c3ccccc3)oc12 ; Cc1cc(C(C)Nc2cccnc2C(N)=O)c2oc(-c3ccccc3)c(C)c(=O)c2c1 ; Cc1cc(C(C)Nc2ccc(Cl)nc2C(N)=O)c2oc(-c3ccccc3)c(C)c(=O)c2c1]

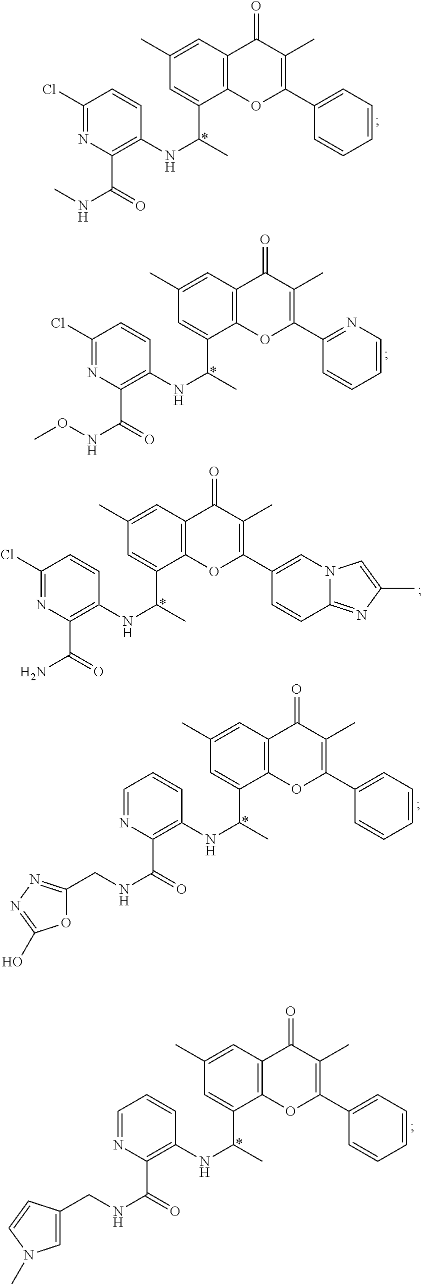 [SMILES: CNC(=O)c1nc(Cl)ccc1NC(C)c1cc(C)cc2c(=O)c(C)c(-c3ccccc3)oc12 ; CONC(=O)c1nc(Cl)ccc1NC(C)c1cc(C)cc2c(=O)c(C)c(-c3ccccn3)oc12 ; Cc1cc(C(C)Nc2ccc(Cl)nc2C(N)=O)c2oc(-c3ccc4nc(C)cn4c3)c(C)c(=O)c2c1 ; Cc1cc(C(C)Nc2cccnc2C(=O)NCc2nnc(O)o2)c2oc(-c3ccccc3)c(C)c(=O)c2c1 ; Cc1cc(C(C)Nc2cccnc2C(=O)NCc2ccn(C)c2)c2oc(-c3ccccc3)c(C)c(=O)c2c1]

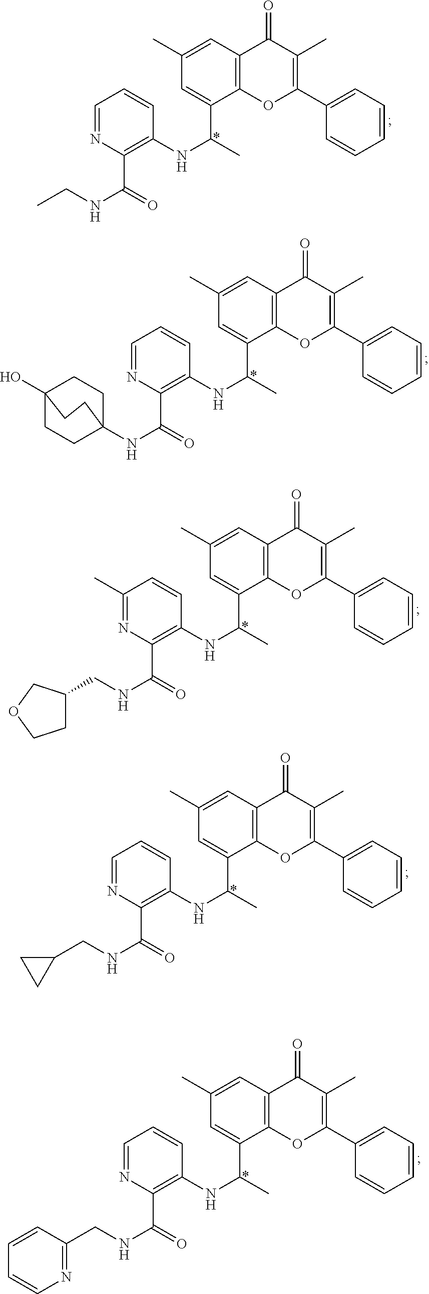 [SMILES: CCNC(=O)c1ncccc1NC(C)c1cc(C)cc2c(=O)c(C)c(-c3ccccc3)oc12 ; Cc1cc(C(C)Nc2cccnc2C(=O)NC23CCC(O)(CC2)CC3)c2oc(-c3ccccc3)c(C)c(=O)c2c1 ; Cc1cc(C(C)Nc2ccc(C)nc2C(=O)NC[C@@H]2CCOC2)c2oc(-c3ccccc3)c(C)c(=O)c2c1 ; Cc1cc(C(C)Nc2cccnc2C(=O)NCC2CC2)c2oc(-c3ccccc3)c(C)c(=O)c2c1 ; Cc1cc(C(C)Nc2cccnc2C(=O)NCc2ccccn2)c2oc(-c3ccccc3)c(C)c(=O)c2c1]

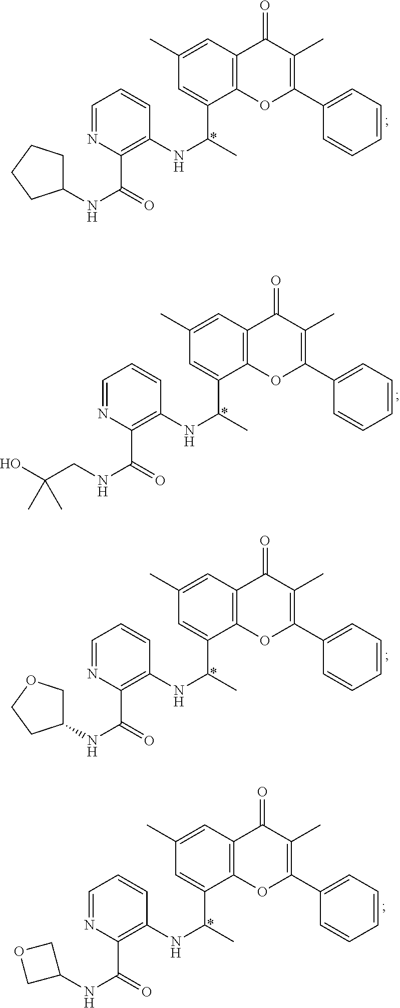 [SMILES: Cc1cc(C(C)Nc2cccnc2C(=O)NC2CCCC2)c2oc(-c3ccccc3)c(C)c(=O)c2c1 ; Cc1cc(C(C)Nc2cccnc2C(=O)NCC(C)(C)O)c2oc(-c3ccccc3)c(C)c(=O)c2c1 ; Cc1cc(C(C)Nc2cccnc2C(=O)N[C@@H]2CCOC2)c2oc(-c3ccccc3)c(C)c(=O)c2c1 ; Cc1cc(C(C)Nc2cccnc2C(=O)NC2COC2)c2oc(-c3ccccc3)c(C)c(=O)c2c1]

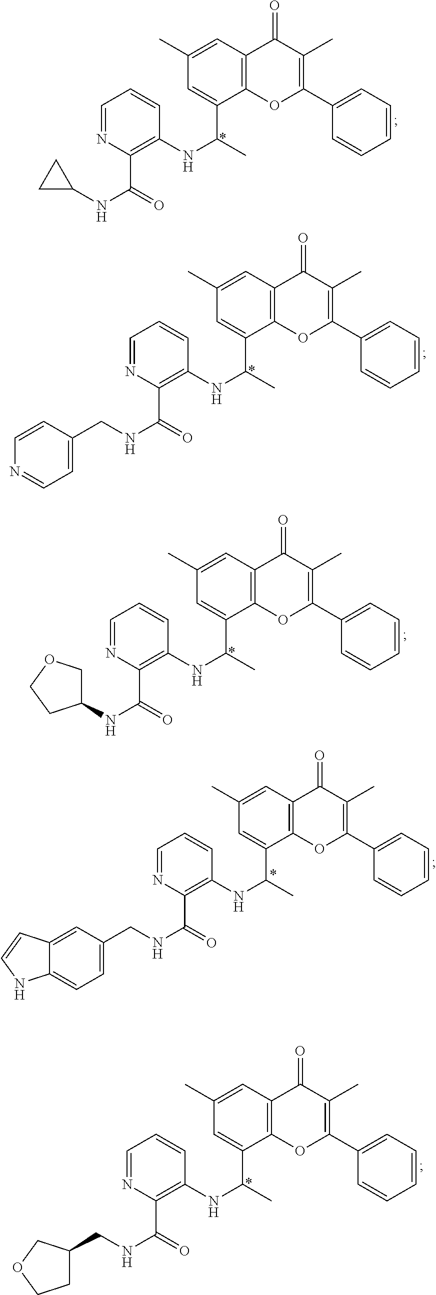 [SMILES: Cc1cc(C(C)Nc2cccnc2C(=O)NC2CC2)c2oc(-c3ccccc3)c(C)c(=O)c2c1 ; Cc1cc(C(C)Nc2cccnc2C(=O)NCc2ccncc2)c2oc(-c3ccccc3)c(C)c(=O)c2c1 ; Cc1cc(C(C)Nc2cccnc2C(=O)N[C@H]2CCOC2)c2oc(-c3ccccc3)c(C)c(=O)c2c1 ; Cc1cc(C(C)Nc2cccnc2C(=O)NCc2ccc3[nH]ccc3c2)c2oc(-c3ccccc3)c(C)c(=O)c2c1 ; Cc1cc(C(C)Nc2cccnc2C(=O)NC[C@H]2CCOC2)c2oc(-c3ccccc3)c(C)c(=O)c2c1]

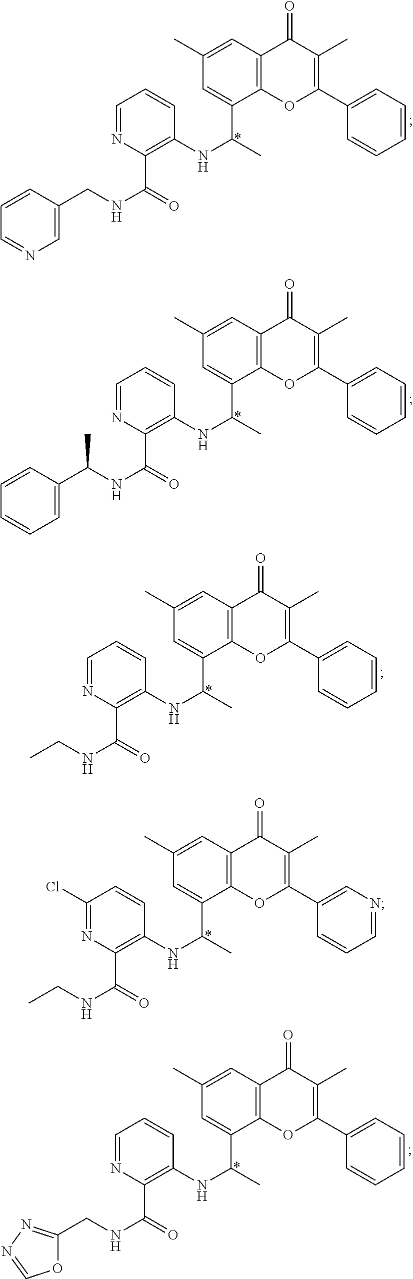 [SMILES: Cc1cc(C(C)Nc2cccnc2C(=O)NCc2cccnc2)c2oc(-c3ccccc3)c(C)c(=O)c2c1 ; Cc1cc(C(C)Nc2cccnc2C(=O)N[C@H](C)c2ccccc2)c2oc(-c3ccccc3)c(C)c(=O)c2c1 ; CCNC(=O)c1ncccc1NC(C)c1cc(C)cc2c(=O)c(C)c(-c3ccccc3)oc12 ; CCNC(=O)c1nc(Cl)ccc1NC(C)c1cc(C)cc2c(=O)c(C)c(-c3cccnc3)oc12 ; Cc1cc(C(C)Nc2cccnc2C(=O)NCc2nnco2)c2oc(-c3ccccc3)c(C)c(=O)c2c1]

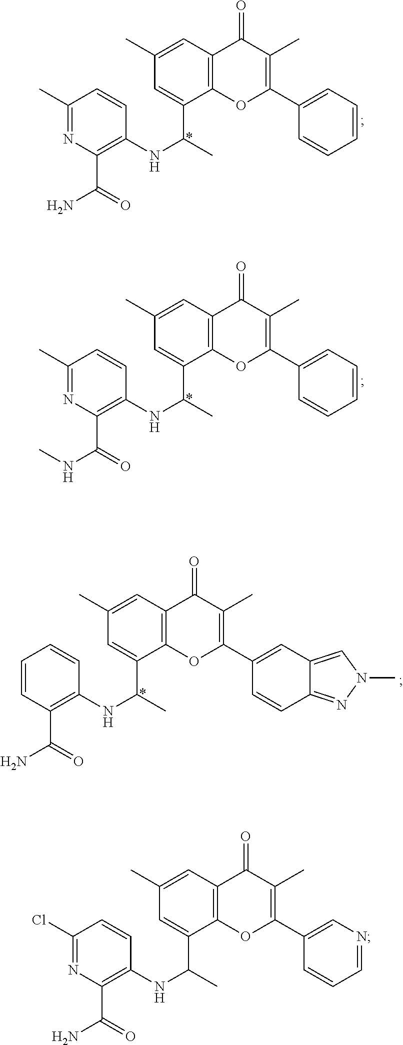 [SMILES: Cc1cc(C(C)Nc2ccc(C)nc2C(N)=O)c2oc(-c3ccccc3)c(C)c(=O)c2c1 ; CNC(=O)c1nc(C)ccc1NC(C)c1cc(C)cc2c(=O)c(C)c(-c3ccccc3)oc12 ; Cc1cc(C(C)Nc2ccccc2C(N)=O)c2oc(-c3ccc4nn(C)cc4c3)c(C)c(=O)c2c1 ; Cc1cc(C(C)Nc2ccc(Cl)nc2C(N)=O)c2oc(-c3cccnc3)c(C)c(=O)c2c1]

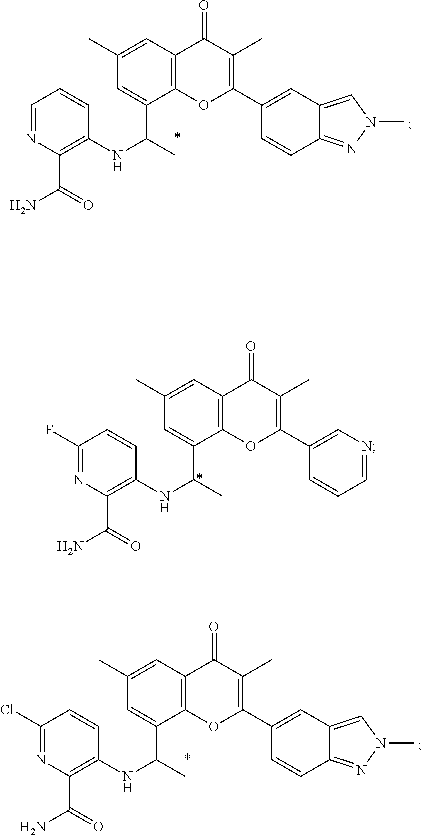 [SMILES: Cc1cc(C(C)Nc2cccnc2C(N)=O)c2oc(-c3ccc4nn(C)cc4c3)c(C)c(=O)c2c1 ; Cc1cc(C(C)Nc2ccc(F)nc2C(N)=O)c2oc(-c3cccnc3)c(C)c(=O)c2c1 ; Cc1cc(C(C)Nc2ccc(Cl)nc2C(N)=O)c2oc(-c3ccc4nn(C)cc4c3)c(C)c(=O)c2c1]

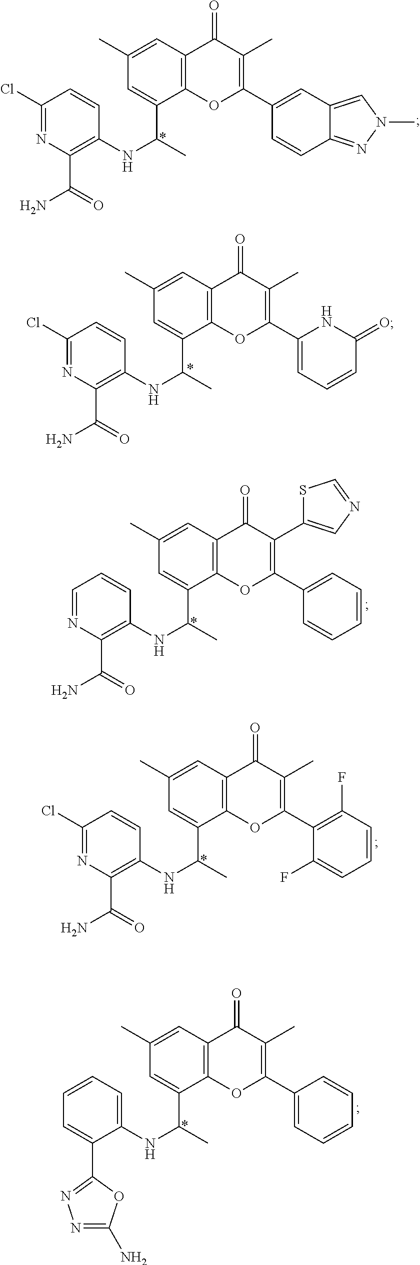 [SMILES: Cc1cc(C(C)Nc2ccc(Cl)nc2C(N)=O)c2oc(-c3ccc4nn(C)cc4c3)c(C)c(=O)c2c1 ; Cc1cc(C(C)Nc2ccc(Cl)nc2C(N)=O)c2oc(-c3cccc(=O)[nH]3)c(C)c(=O)c2c1 ; Cc1cc(C(C)Nc2cccnc2C(N)=O)c2oc(-c3ccccc3)c(-c3cncs3)c(=O)c2c1 ; Cc1cc(C(C)Nc2ccc(Cl)nc2C(N)=O)c2oc(-c3c(F)cccc3F)c(C)c(=O)c2c1 ; Cc1cc(C(C)Nc2ccccc2-c2nnc(N)o2)c2oc(-c3ccccc3)c(C)c(=O)c2c1]

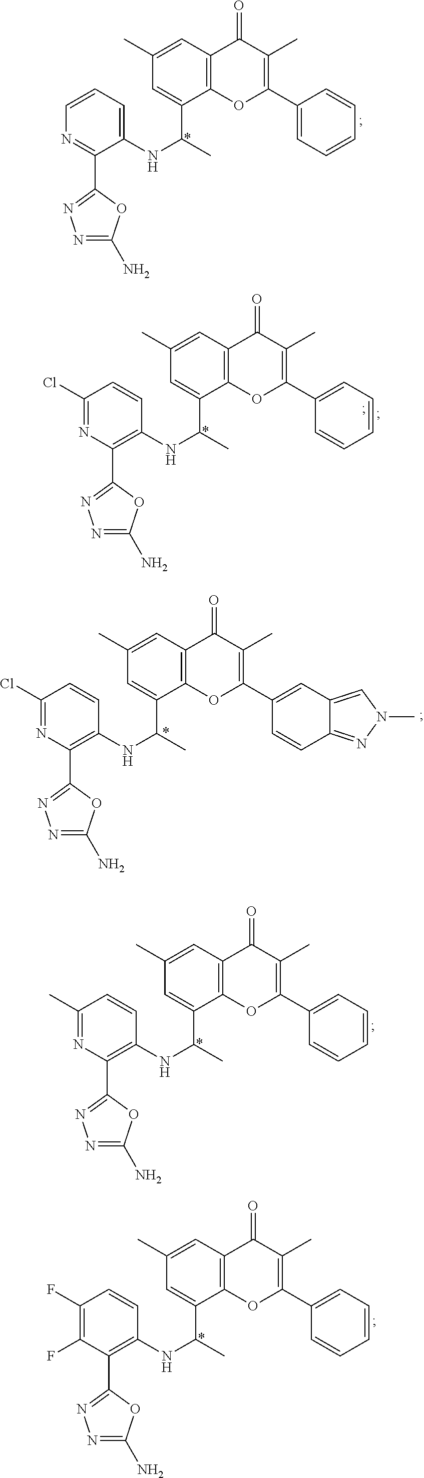

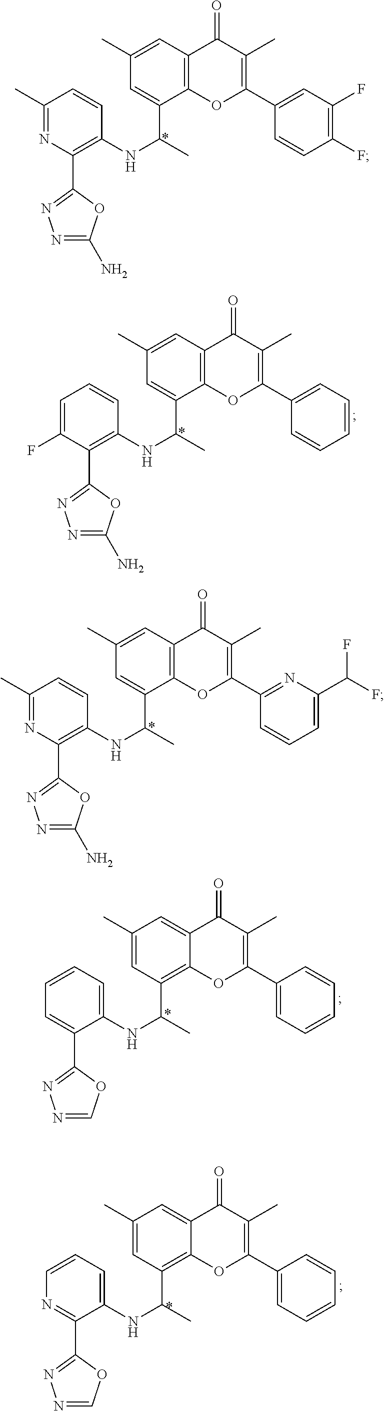 [SMILES: Cc1cc(C(C)Nc2ccc(C)nc2-c2nnc(N)o2)c2oc(-c3ccc(F)c(F)c3)c(C)c(=O)c2c1 ; Cc1cc(C(C)Nc2cccc(F)c2-c2nnc(N)o2)c2oc(-c3ccccc3)c(C)c(=O)c2c1 ; Cc1cc(C(C)Nc2ccc(C)nc2-c2nnc(N)o2)c2oc(-c3cccc(C(F)F)n3)c(C)c(=O)c2c1 ; Cc1cc(C(C)Nc2ccccc2-c2nnco2)c2oc(-c3ccccc3)c(C)c(=O)c2c1 ; Cc1cc(C(C)Nc2cccnc2-c2nnco2)c2oc(-c3ccccc3)c(C)c(=O)c2c1]

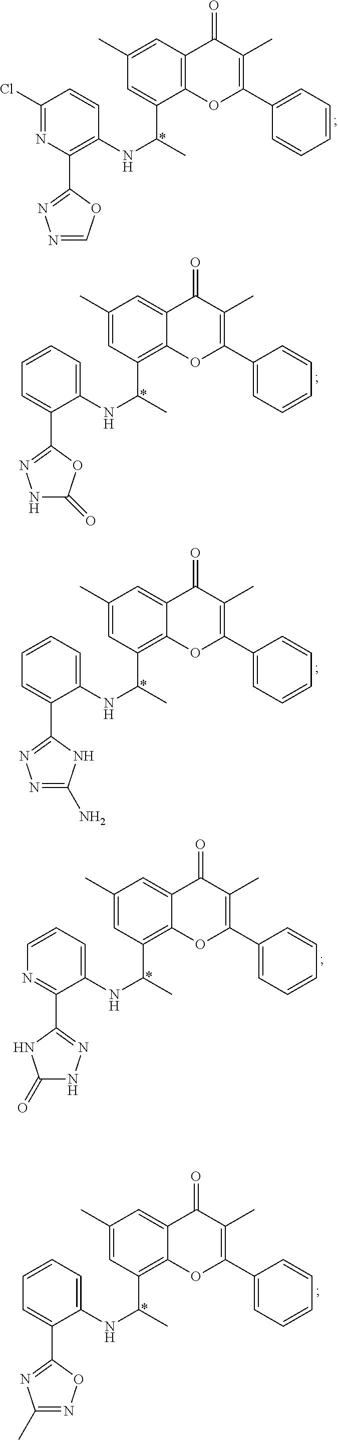 [SMILES: Cc1cc(C(C)Nc2ccc(Cl)nc2-c2nnco2)c2oc(-c3ccccc3)c(C)c(=O)c2c1 ; Cc1cc(C(C)Nc2ccccc2-c2n[nH]c(=O)o2)c2oc(-c3ccccc3)c(C)c(=O)c2c1 ; Cc1cc(C(C)Nc2ccccc2-c2nnc(N)[nH]2)c2oc(-c3ccccc3)c(C)c(=O)c2c1 ; Cc1cc(C(C)Nc2cccnc2-c2n[nH]c(=O)[nH]2)c2oc(-c3ccccc3)c(C)c(=O)c2c1 ; Cc1cc(C(C)Nc2ccccc2-c2nc(C)no2)c2oc(-c3ccccc3)c(C)c(=O)c2c1]

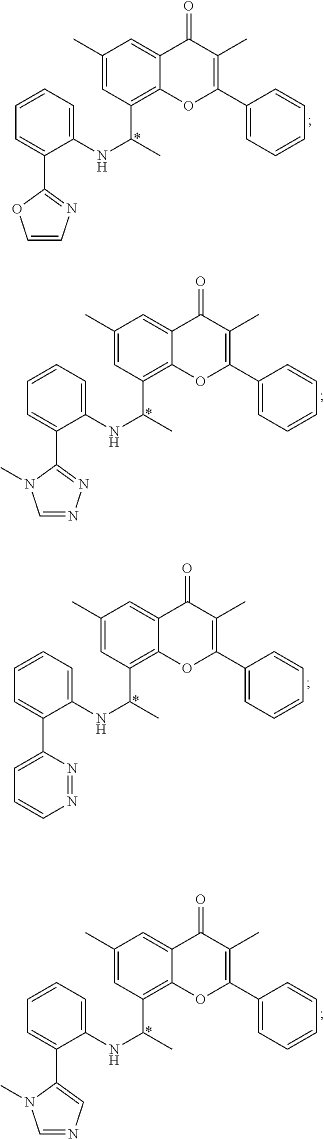 [SMILES: Cc1cc(C(C)Nc2ccccc2-c2ncco2)c2oc(-c3ccccc3)c(C)c(=O)c2c1 ; Cc1cc(C(C)Nc2ccccc2-c2nncn2C)c2oc(-c3ccccc3)c(C)c(=O)c2c1 ; Cc1cc(C(C)Nc2ccccc2-c2cccnn2)c2oc(-c3ccccc3)c(C)c(=O)c2c1 ; Cc1cc(C(C)Nc2ccccc2-c2cncn2C)c2oc(-c3ccccc3)c(C)c(=O)c2c1]

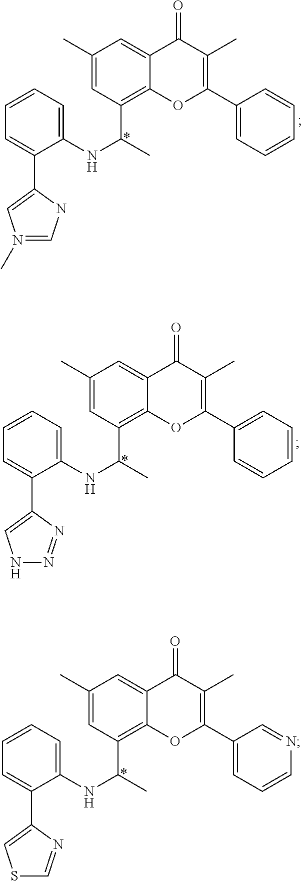

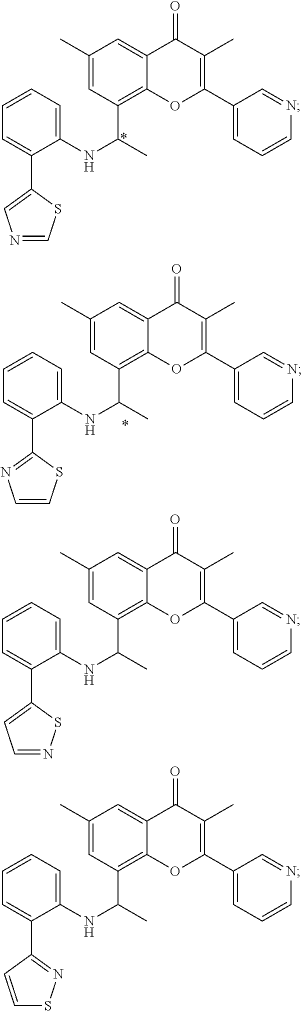 [SMILES: Cc1cc(C(C)Nc2ccccc2-c2cncs2)c2oc(-c3cccnc3)c(C)c(=O)c2c1 ; Cc1cc(C(C)Nc2ccccc2-c2nccs2)c2oc(-c3cccnc3)c(C)c(=O)c2c1 ; Cc1cc(C(C)Nc2ccccc2-c2ccns2)c2oc(-c3cccnc3)c(C)c(=O)c2c1 ; Cc1cc(C(C)Nc2ccccc2-c2ccsn2)c2oc(-c3cccnc3)c(C)c(=O)c2c1]

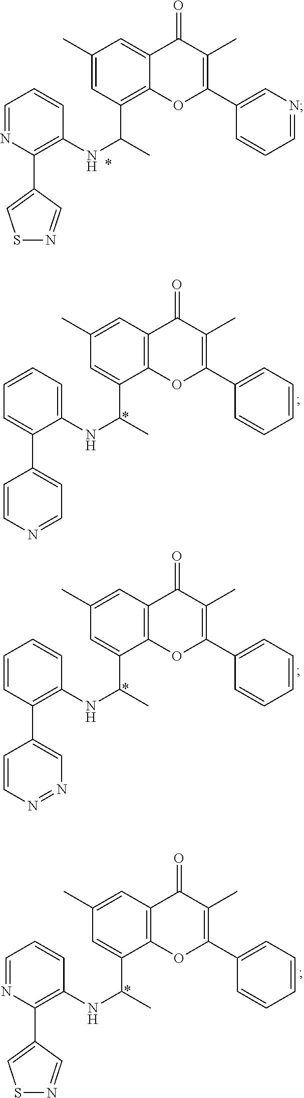 [SMILES: Cc1cc(C(C)Nc2cccnc2-c2cnsc2)c2oc(-c3cccnc3)c(C)c(=O)c2c1 ; Cc1cc(C(C)Nc2ccccc2-c2ccncc2)c2oc(-c3ccccc3)c(C)c(=O)c2c1 ; Cc1cc(C(C)Nc2ccccc2-c2ccnnc2)c2oc(-c3ccccc3)c(C)c(=O)c2c1 ; Cc1cc(C(C)Nc2cccnc2-c2cnsc2)c2oc(-c3ccccc3)c(C)c(=O)c2c1]

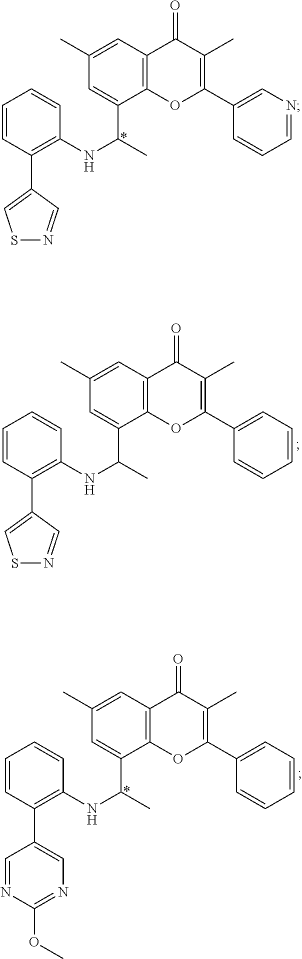 [SMILES: Cc1cc(C(C)Nc2ccccc2-c2cnsc2)c2oc(-c3cccnc3)c(C)c(=O)c2c1 ; Cc1cc(C(C)Nc2ccccc2-c2cnsc2)c2oc(-c3ccccc3)c(C)c(=O)c2c1 ; COc1ncc(-c2ccccc2NC(C)c2cc(C)cc3c(=O)c(C)c(-c4ccccc4)oc23)cn1]

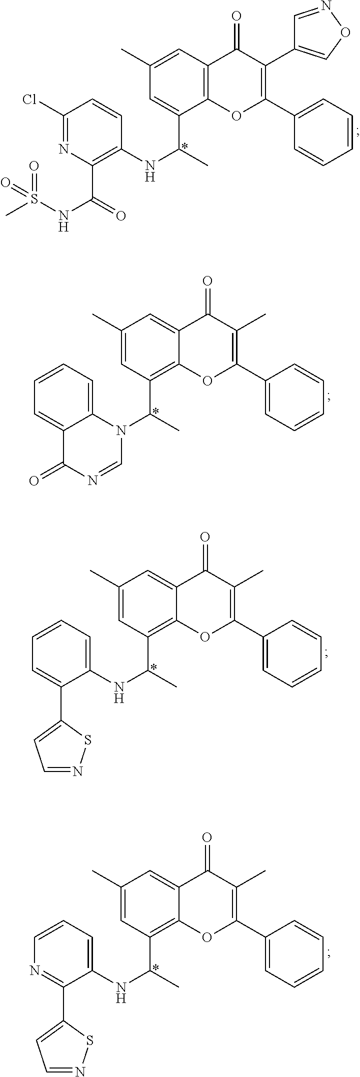 [SMILES: Cc1cc(C(C)Nc2ccc(Cl)nc2C(=O)NS(C)(=O)=O)c2oc(-c3ccccc3)c(-c3cnoc3)c(=O)c2c1 ; Cc1cc(C(C)n2cnc(=O)c3ccccc32)c2oc(-c3ccccc3)c(C)c(=O)c2c1 ; Cc1cc(C(C)Nc2ccccc2-c2ccns2)c2oc(-c3ccccc3)c(C)c(=O)c2c1 ; Cc1cc(C(C)Nc2cccnc2-c2ccns2)c2oc(-c3ccccc3)c(C)c(=O)c2c1]

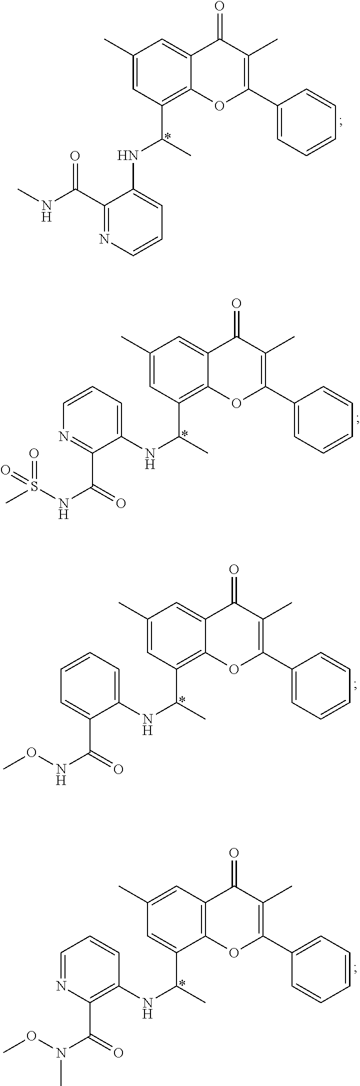 [SMILES: CNC(=O)c1ncccc1NC(C)c1cc(C)cc2c(=O)c(C)c(-c3ccccc3)oc12 ; Cc1cc(C(C)Nc2cccnc2C(=O)NS(C)(=O)=O)c2oc(-c3ccccc3)c(C)c(=O)c2c1 ; CONC(=O)c1ccccc1NC(C)c1cc(C)cc2c(=O)c(C)c(-c3ccccc3)oc12 ; CON(C)C(=O)c1ncccc1NC(C)c1cc(C)cc2c(=O)c(C)c(-c3ccccc3)oc12]

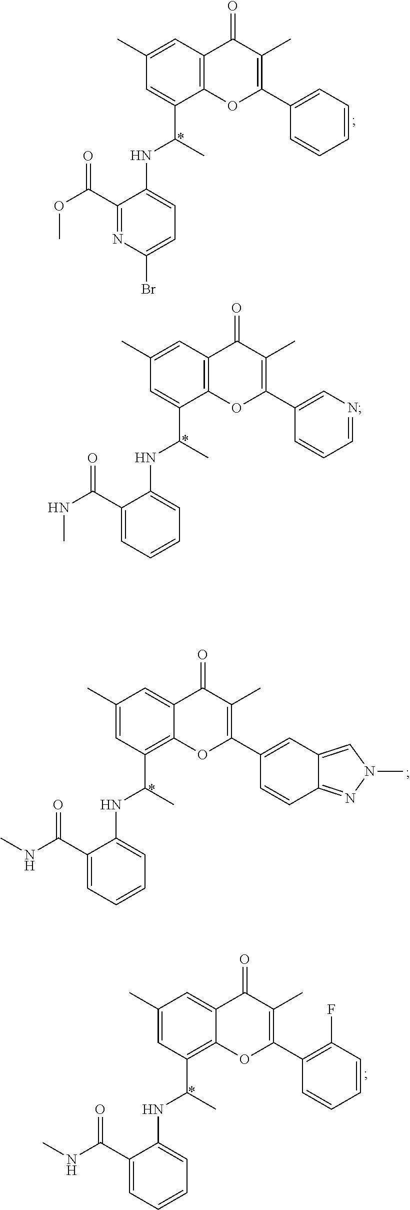

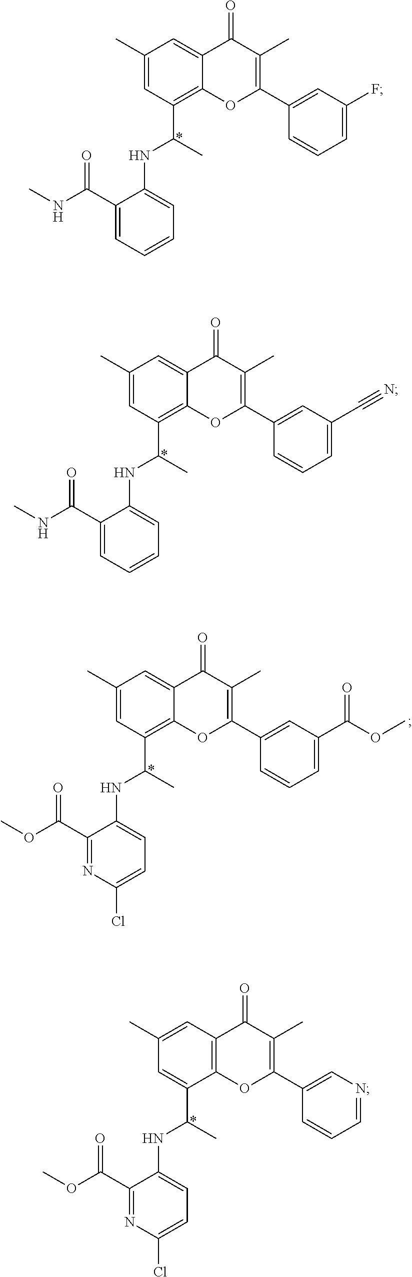

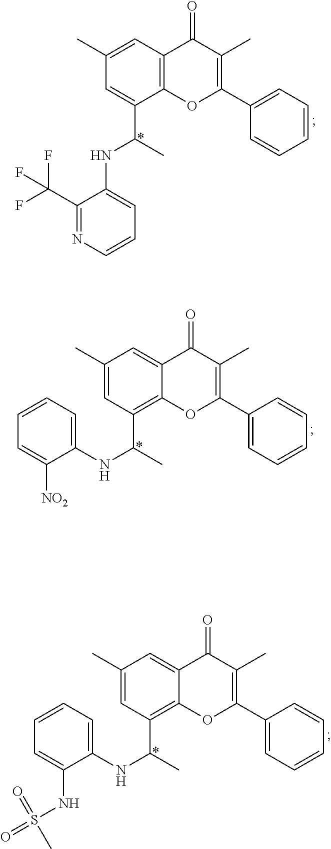 [SMILES: Cc1cc(C(C)Nc2cccnc2C(F)(F)F)c2oc(-c3ccccc3)c(C)c(=O)c2c1 ; Cc1cc(C(C)Nc2ccccc2[N+](=O)[O-])c2oc(-c3ccccc3)c(C)c(=O)c2c1 ; Cc1cc(C(C)Nc2ccccc2NS(C)(=O)=O)c2oc(-c3ccccc3)c(C)c(=O)c2c1]

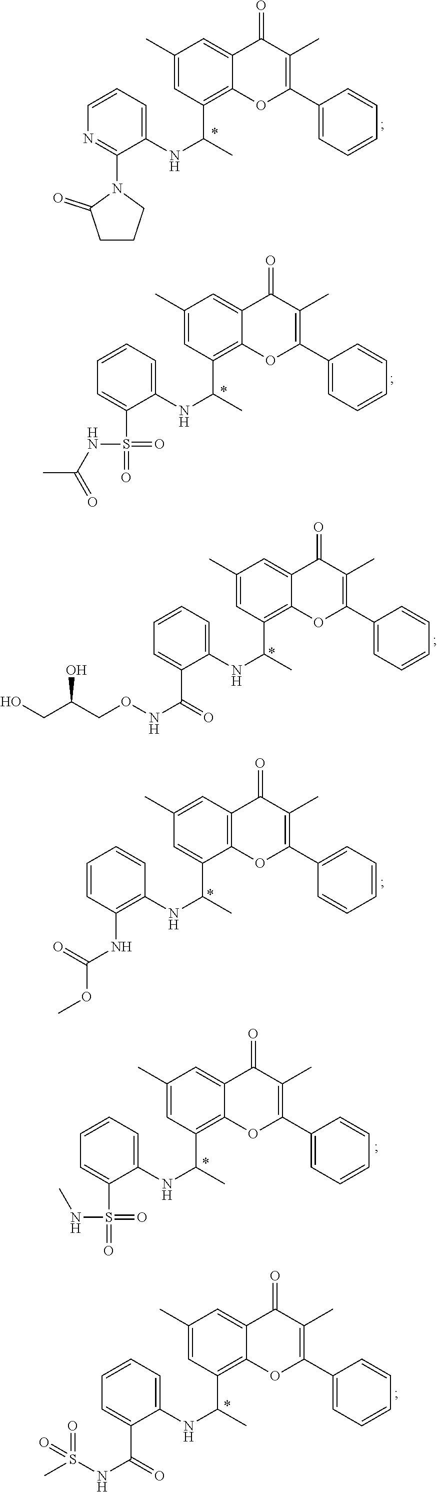 [SMILES: Cc1cc(C(C)Nc2cccnc2N2CCCC2=O)c2oc(-c3ccccc3)c(C)c(=O)c2c1 ; CC(=O)NS(=O)(=O)c1ccccc1NC(C)c1cc(C)cc2c(=O)c(C)c(-c3ccccc3)oc12 ; Cc1cc(C(C)Nc2ccccc2C(=O)NOC[C@H](O)CO)c2oc(-c3ccccc3)c(C)c(=O)c2c1 ; COC(=O)Nc1ccccc1NC(C)c1cc(C)cc2c(=O)c(C)c(-c3ccccc3)oc12 ; CNS(=O)(=O)c1ccccc1NC(C)c1cc(C)cc2c(=O)c(C)c(-c3ccccc3)oc12 ; Cc1cc(C(C)Nc2ccccc2C(=O)NS(C)(=O)=O)c2oc(-c3ccccc3)c(C)c(=O)c2c1]

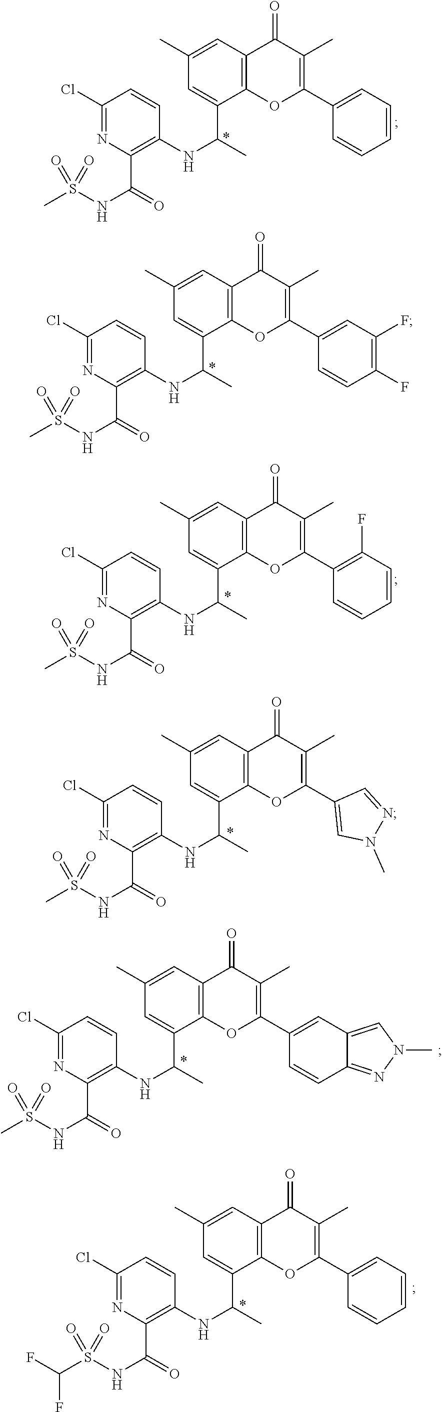

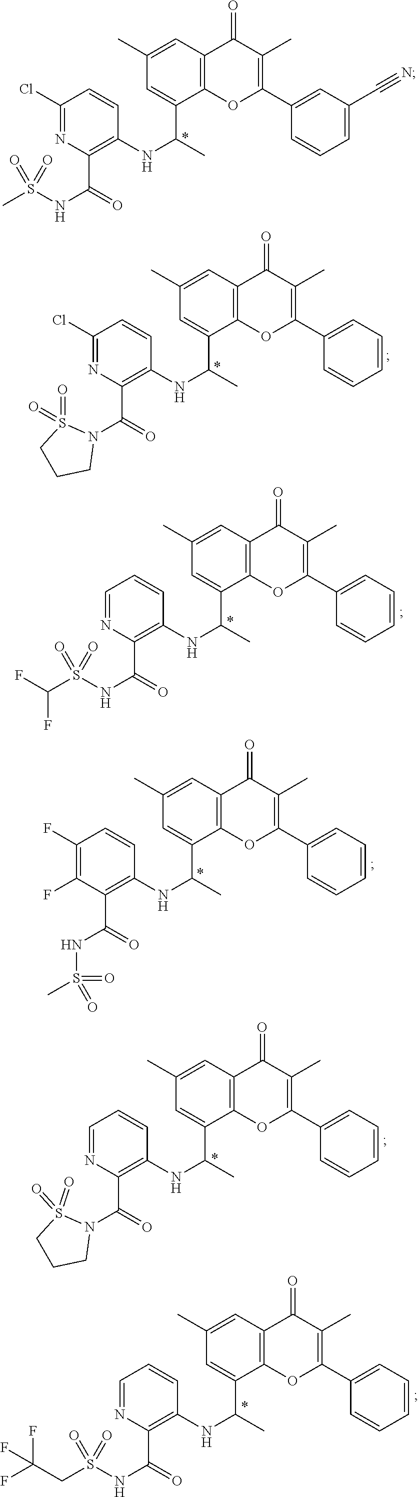 [SMILES: Cc1cc(C(C)Nc2ccc(Cl)nc2C(=O)NS(C)(=O)=O)c2oc(-c3cccc(C#N)c3)c(C)c(=O)c2c1 ; Cc1cc(C(C)Nc2ccc(Cl)nc2C(=O)N2CCCS2(=O)=O)c2oc(-c3ccccc3)c(C)c(=O)c2c1 ; Cc1cc(C(C)Nc2cccnc2C(=O)NS(=O)(=O)C(F)F)c2oc(-c3ccccc3)c(C)c(=O)c2c1 ; Cc1cc(C(C)Nc2ccc(F)c(F)c2C(=O)NS(C)(=O)=O)c2oc(-c3ccccc3)c(C)c(=O)c2c1 ; Cc1cc(C(C)Nc2cccnc2C(=O)N2CCCS2(=O)=O)c2oc(-c3ccccc3)c(C)c(=O)c2c1 ; Cc1cc(C(C)Nc2cccnc2C(=O)NS(=O)(=O)CC(F)(F)F)c2oc(-c3ccccc3)c(C)c(=O)c2c1]

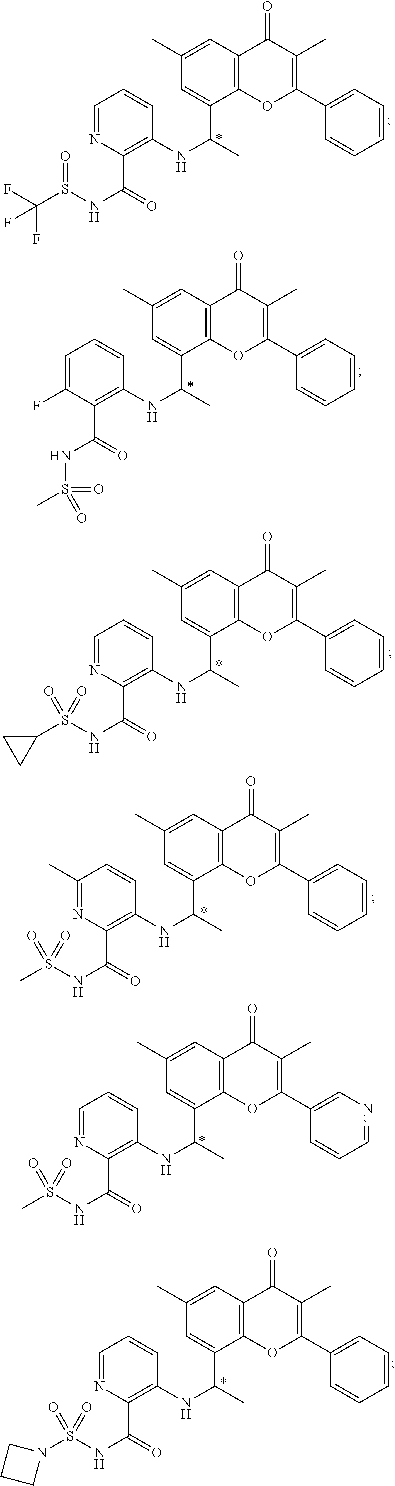 [SMILES: Cc1cc(C(C)Nc2cccnc2C(=O)NS(=O)C(F)(F)F)c2oc(-c3ccccc3)c(C)c(=O)c2c1 ; Cc1cc(C(C)Nc2cccc(F)c2C(=O)NS(C)(=O)=O)c2oc(-c3ccccc3)c(C)c(=O)c2c1 ; Cc1cc(C(C)Nc2cccnc2C(=O)NS(=O)(=O)C2CC2)c2oc(-c3ccccc3)c(C)c(=O)c2c1 ; Cc1cc(C(C)Nc2ccc(C)nc2C(=O)NS(C)(=O)=O)c2oc(-c3ccccc3)c(C)c(=O)c2c1 ; Cc1cc(C(C)Nc2cccnc2C(=O)NS(C)(=O)=O)c2oc(-c3cccnc3)c(C)c(=O)c2c1 ; Cc1cc(C(C)Nc2cccnc2C(=O)NS(=O)(=O)N2CCC2)c2oc(-c3ccccc3)c(C)c(=O)c2c1]

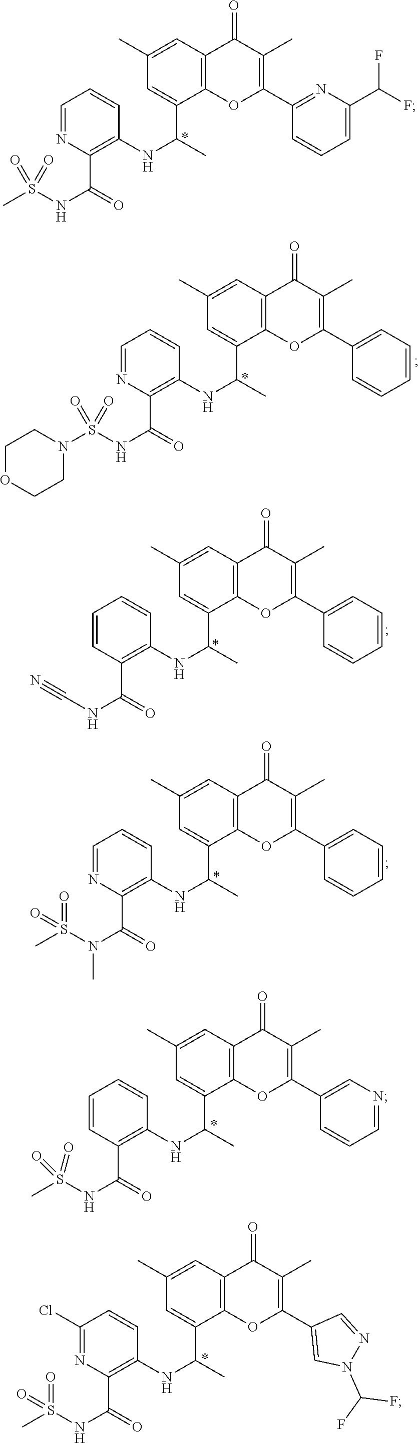 [SMILES: Cc1cc(C(C)Nc2cccnc2C(=O)NS(C)(=O)=O)c2oc(-c3cccc(C(F)F)n3)c(C)c(=O)c2c1 ; Cc1cc(C(C)Nc2cccnc2C(=O)NS(=O)(=O)N2CCOCC2)c2oc(-c3ccccc3)c(C)c(=O)c2c1 ; Cc1cc(C(C)Nc2ccccc2C(=O)NC#N)c2oc(-c3ccccc3)c(C)c(=O)c2c1 ; Cc1cc(C(C)Nc2cccnc2C(=O)N(C)S(C)(=O)=O)c2oc(-c3ccccc3)c(C)c(=O)c2c1 ; Cc1cc(C(C)Nc2ccccc2C(=O)NS(C)(=O)=O)c2oc(-c3cccnc3)c(C)c(=O)c2c1 ; Cc1cc(C(C)Nc2ccc(Cl)nc2C(=O)NS(C)(=O)=O)c2oc(-c3cnn(C(F)F)c3)c(C)c(=O)c2c1]

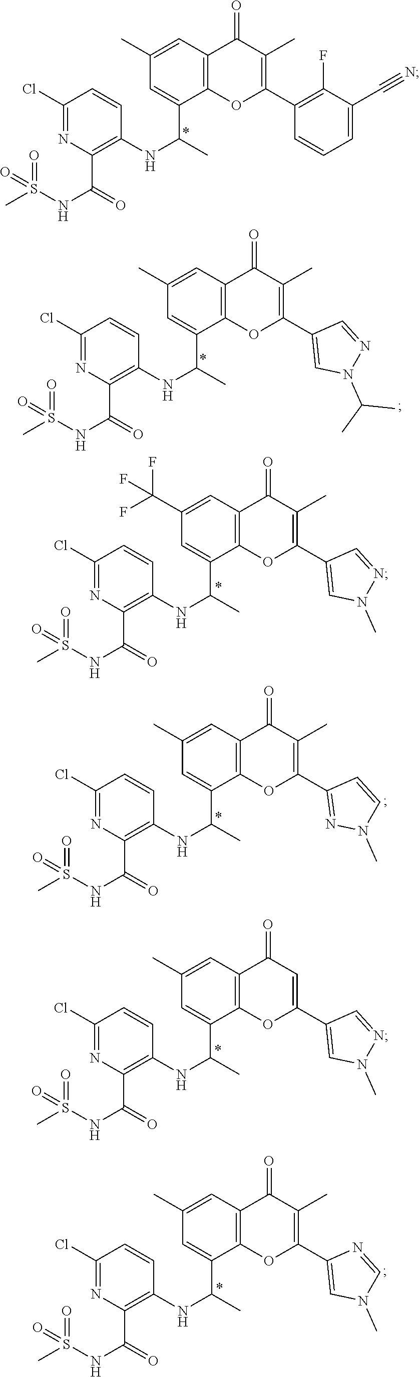

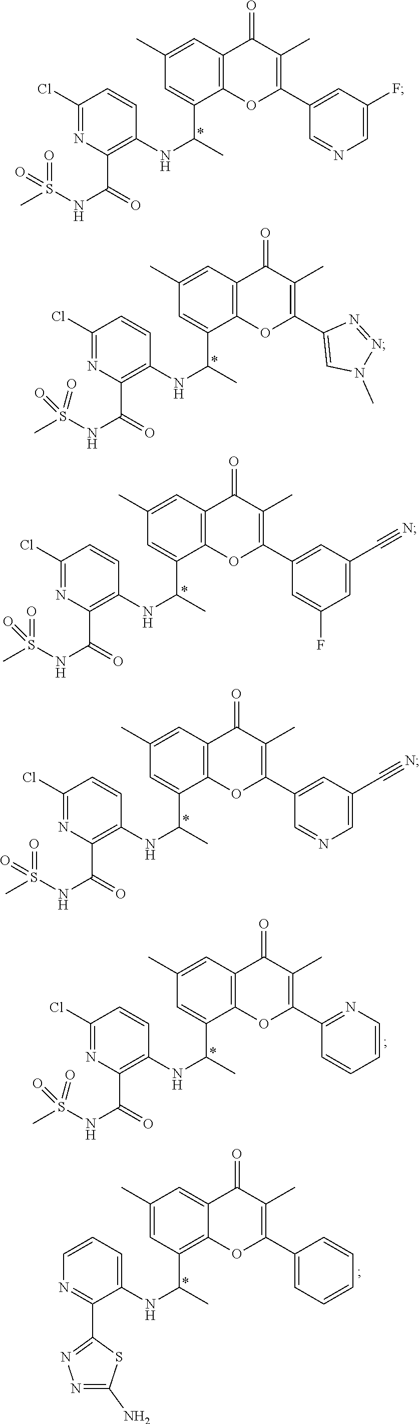 [SMILES: Cc1cc(C(C)Nc2ccc(Cl)nc2C(=O)NS(C)(=O)=O)c2oc(-c3cncc(F)c3)c(C)c(=O)c2c1 ; Cc1cc(C(C)Nc2ccc(Cl)nc2C(=O)NS(C)(=O)=O)c2oc(-c3cn(C)nn3)c(C)c(=O)c2c1 ; Cc1cc(C(C)Nc2ccc(Cl)nc2C(=O)NS(C)(=O)=O)c2oc(-c3cc(F)cc(C#N)c3)c(C)c(=O)c2c1 ; Cc1cc(C(C)Nc2ccc(Cl)nc2C(=O)NS(C)(=O)=O)c2oc(-c3cncc(C#N)c3)c(C)c(=O)c2c1 ; Cc1cc(C(C)Nc2ccc(Cl)nc2C(=O)NS(C)(=O)=O)c2oc(-c3ccccn3)c(C)c(=O)c2c1 ; Cc1cc(C(C)Nc2cccnc2-c2nnc(N)s2)c2oc(-c3ccccc3)c(C)c(=O)c2c1]

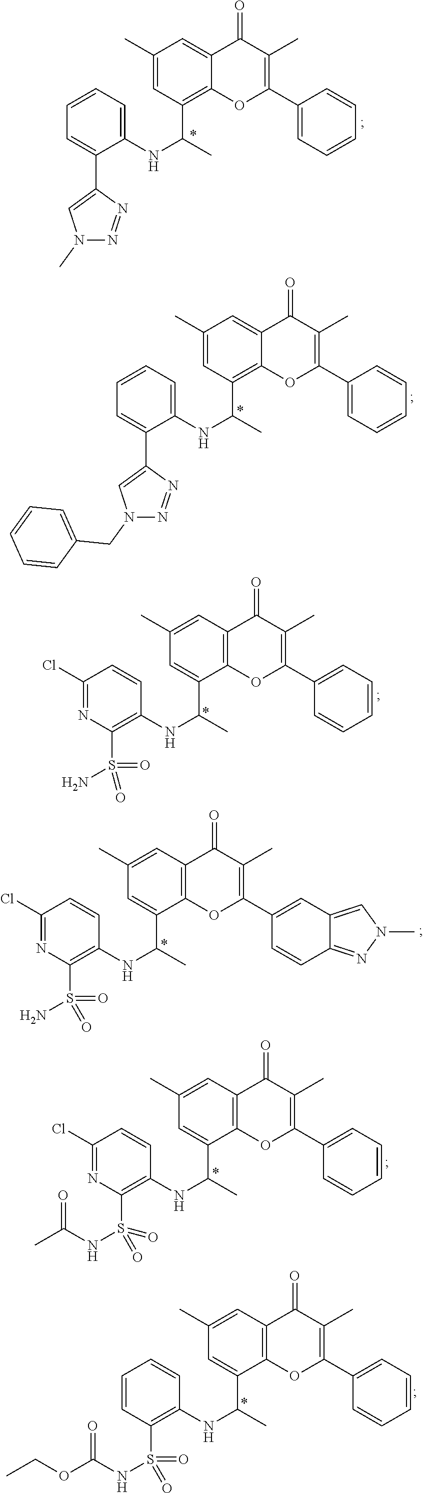

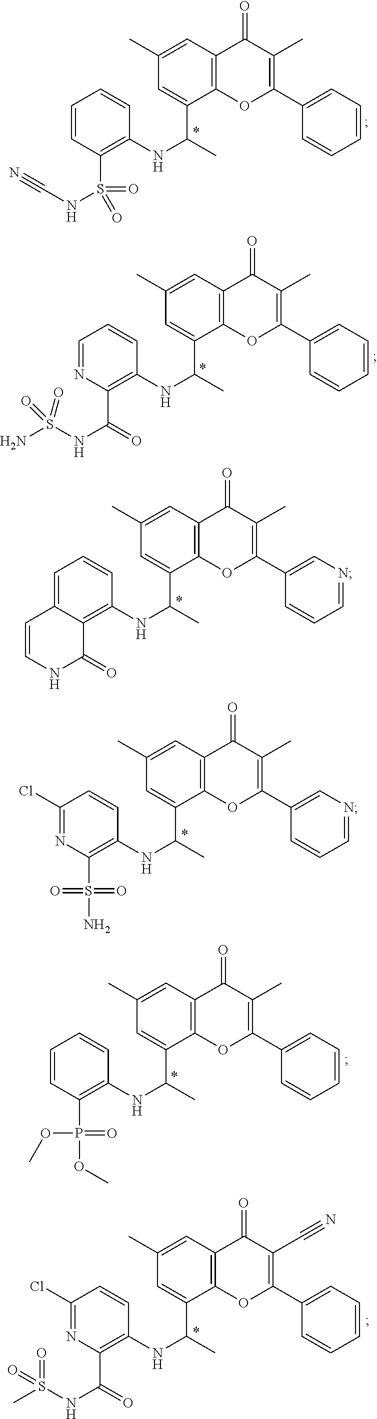 [SMILES: Cc1cc(C(C)Nc2ccccc2S(=O)(=O)NC#N)c2oc(-c3ccccc3)c(C)c(=O)c2c1 ; Cc1cc(C(C)Nc2cccnc2C(=O)NS(N)(=O)=O)c2oc(-c3ccccc3)c(C)c(=O)c2c1 ; Cc1cc(C(C)Nc2cccc3cc[nH]c(=O)c23)c2oc(-c3cccnc3)c(C)c(=O)c2c1 ; Cc1cc(C(C)Nc2ccc(Cl)nc2S(N)(=O)=O)c2oc(-c3cccnc3)c(C)c(=O)c2c1 ; COP(=O)(OC)c1ccccc1NC(C)c1cc(C)cc2c(=O)c(C)c(-c3ccccc3)oc12 ; Cc1cc(C(C)Nc2ccc(Cl)nc2C(=O)NS(C)(=O)=O)c2oc(-c3ccccc3)c(C#N)c(=O)c2c1]

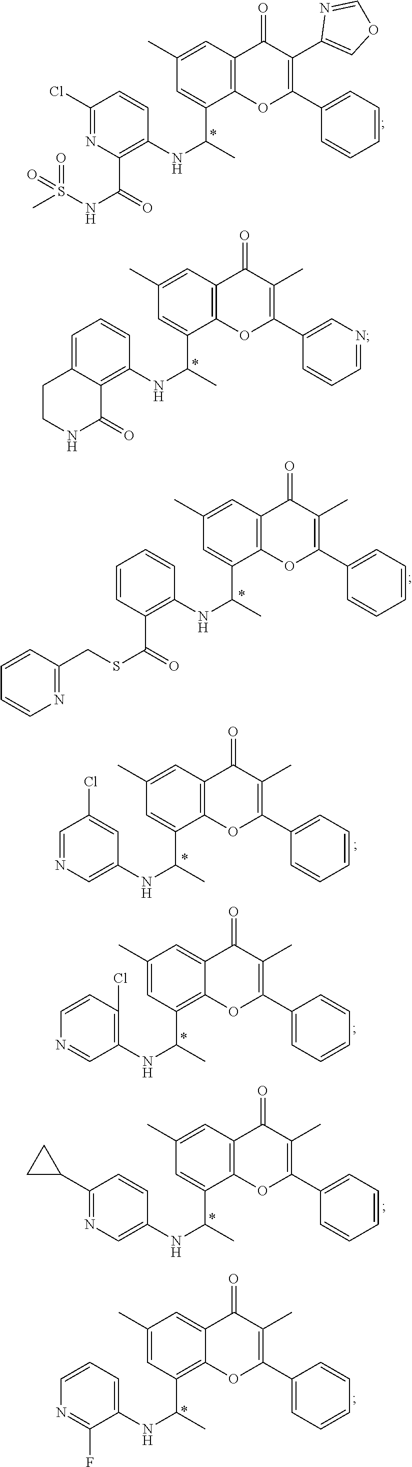

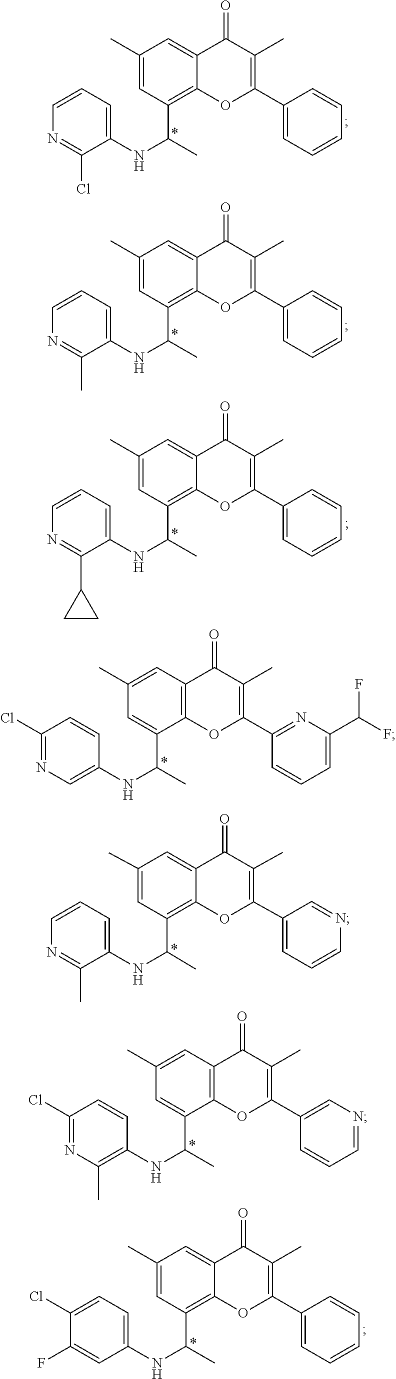

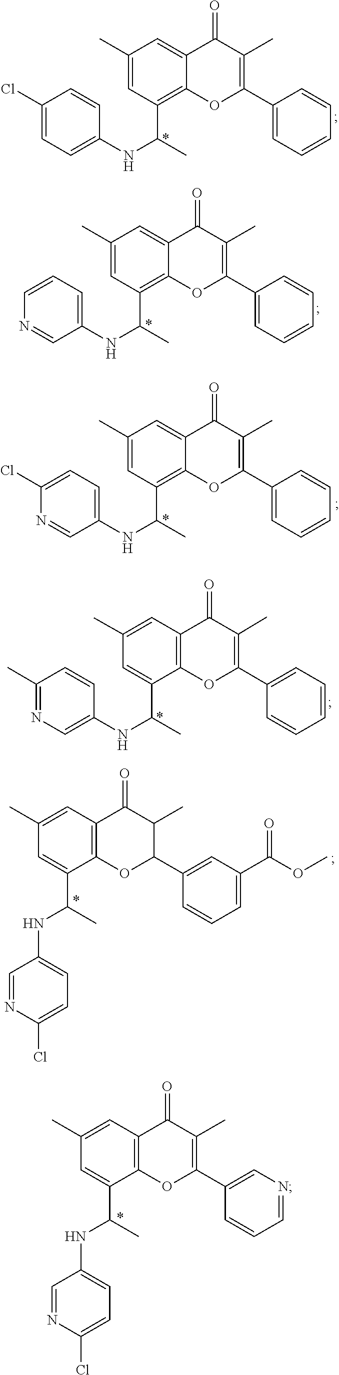

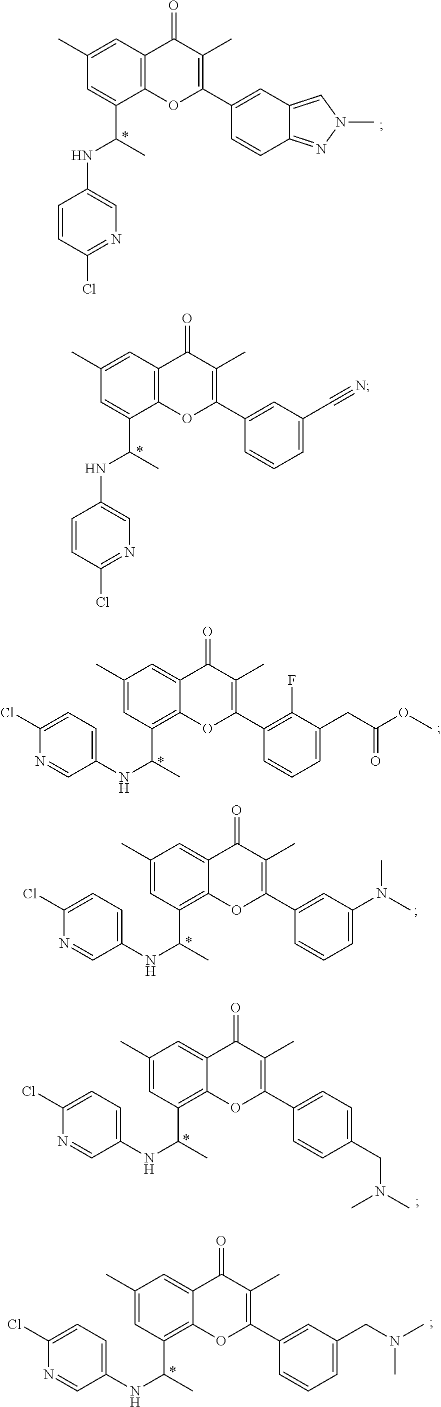 [SMILES: Cc1cc(C(C)Nc2ccc(Cl)nc2)c2oc(-c3ccc4nn(C)cc4c3)c(C)c(=O)c2c1 ; Cc1cc(C(C)Nc2ccc(Cl)nc2)c2oc(-c3cccc(C#N)c3)c(C)c(=O)c2c1 ; COC(=O)Cc1cccc(-c2oc3c(C(C)Nc4ccc(Cl)nc4)cc(C)cc3c(=O)c2C)c1F ; Cc1cc(C(C)Nc2ccc(Cl)nc2)c2oc(-c3cccc(N(C)C)c3)c(C)c(=O)c2c1 ; Cc1cc(C(C)Nc2ccc(Cl)nc2)c2oc(-c3ccc(CN(C)C)cc3)c(C)c(=O)c2c1 ; Cc1cc(C(C)Nc2ccc(Cl)nc2)c2oc(-c3cccc(CN(C)C)c3)c(C)c(=O)c2c1]

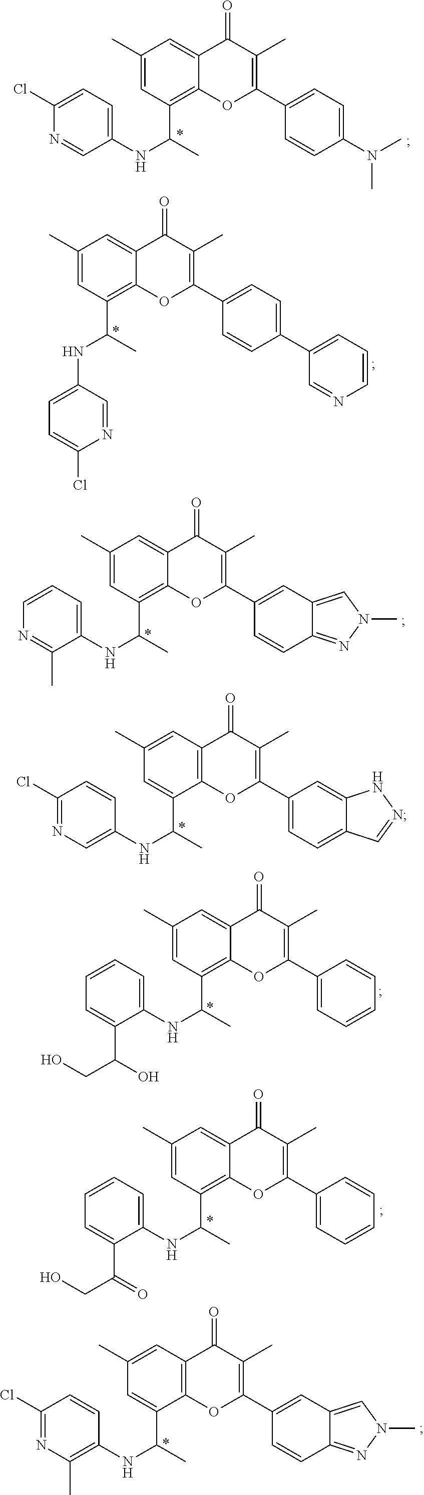

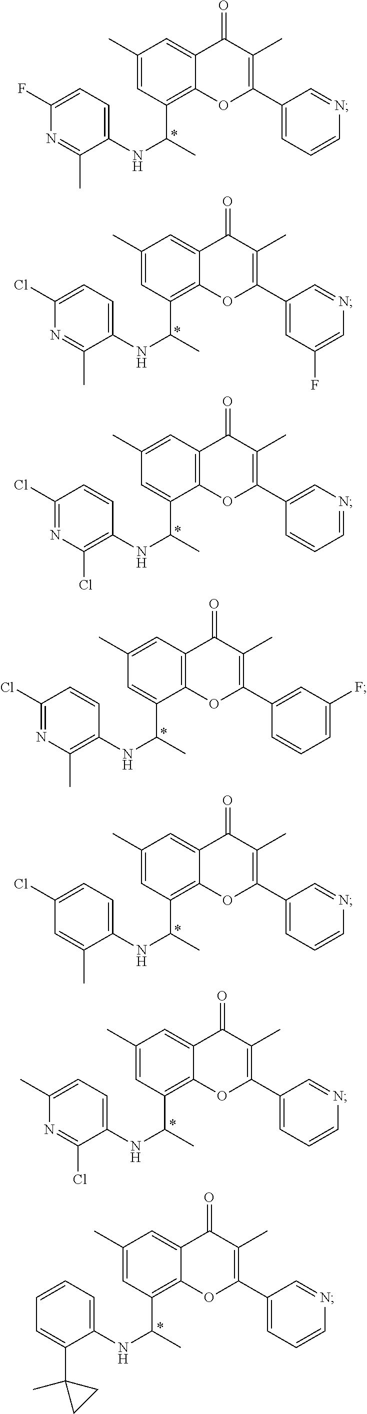

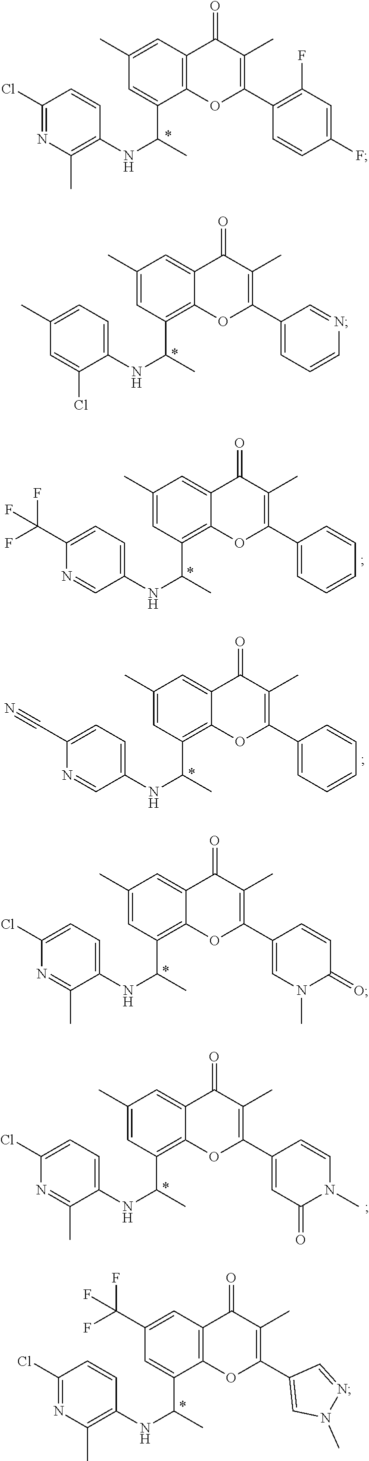

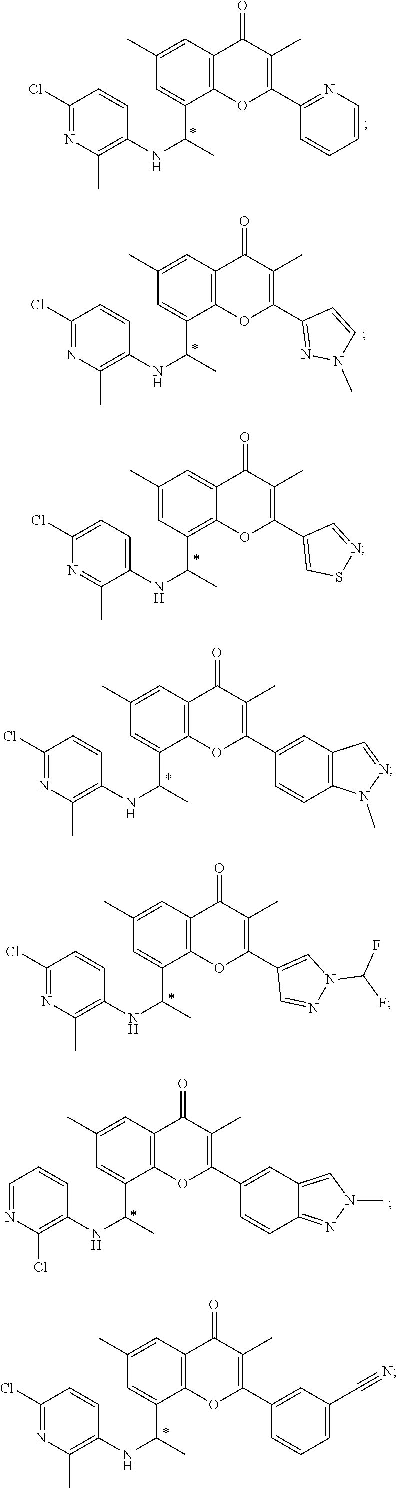

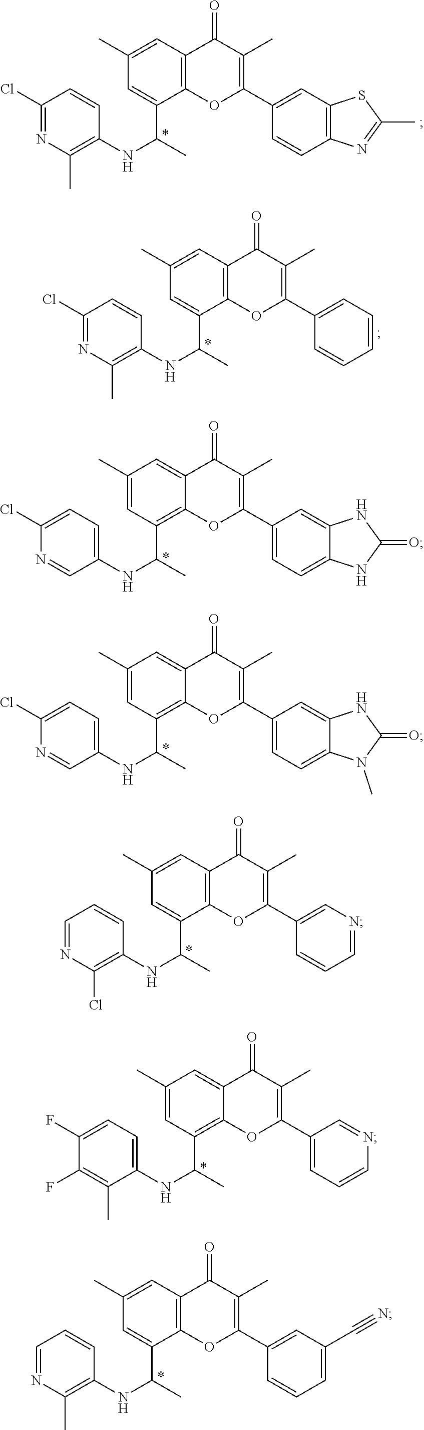

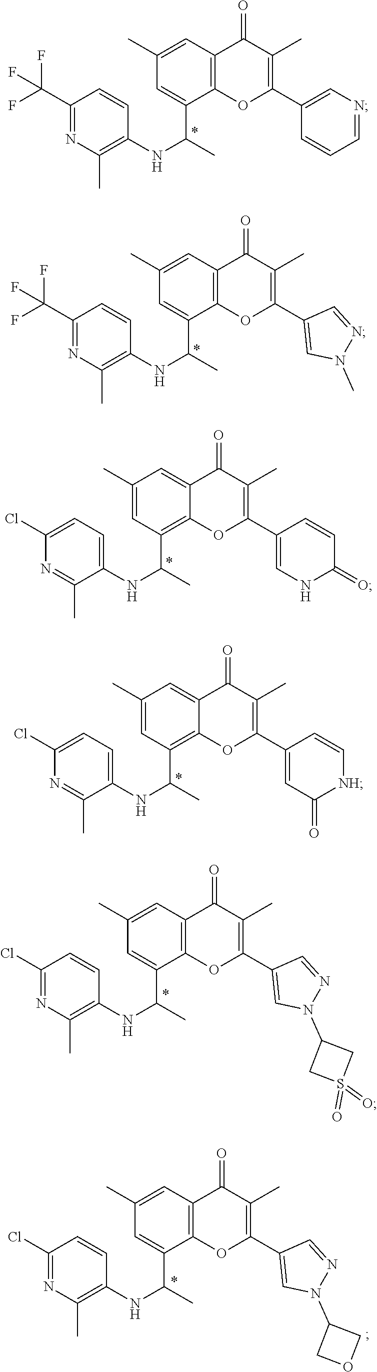 [SMILES: Cc1cc(C(C)Nc2ccc(C(F)(F)F)nc2C)c2oc(-c3cccnc3)c(C)c(=O)c2c1 ; Cc1cc(C(C)Nc2ccc(C(F)(F)F)nc2C)c2oc(-c3cnn(C)c3)c(C)c(=O)c2c1 ; Cc1cc(C(C)Nc2ccc(Cl)nc2C)c2oc(-c3ccc(=O)[nH]c3)c(C)c(=O)c2c1 ; Cc1cc(C(C)Nc2ccc(Cl)nc2C)c2oc(-c3cc[nH]c(=O)c3)c(C)c(=O)c2c1 ; Cc1cc(C(C)Nc2ccc(Cl)nc2C)c2oc(-c3cnn(C4CS(=O)(=O)C4)c3)c(C)c(=O)c2c1 ; Cc1cc(C(C)Nc2ccc(Cl)nc2C)c2oc(-c3cnn(C4COC4)c3)c(C)c(=O)c2c1]

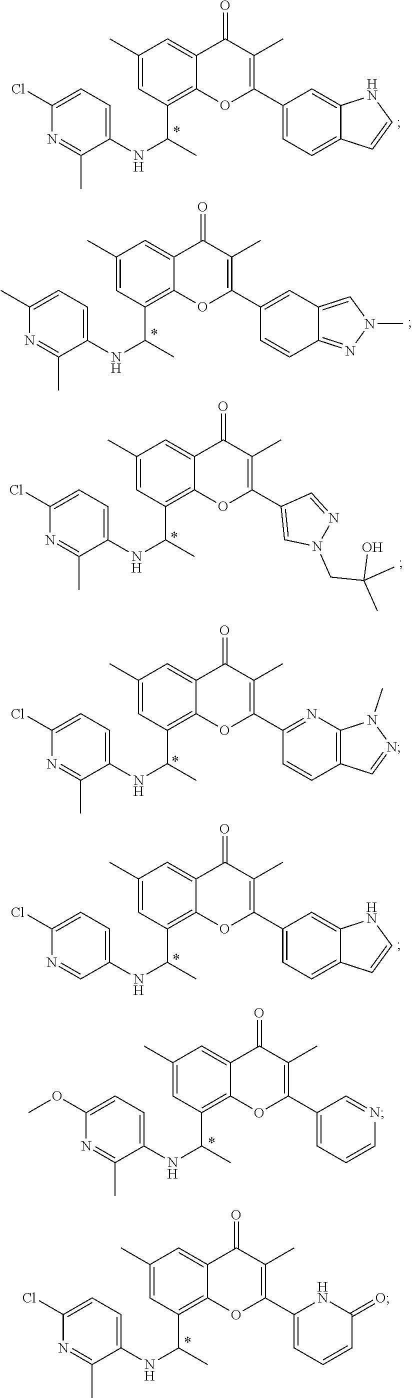

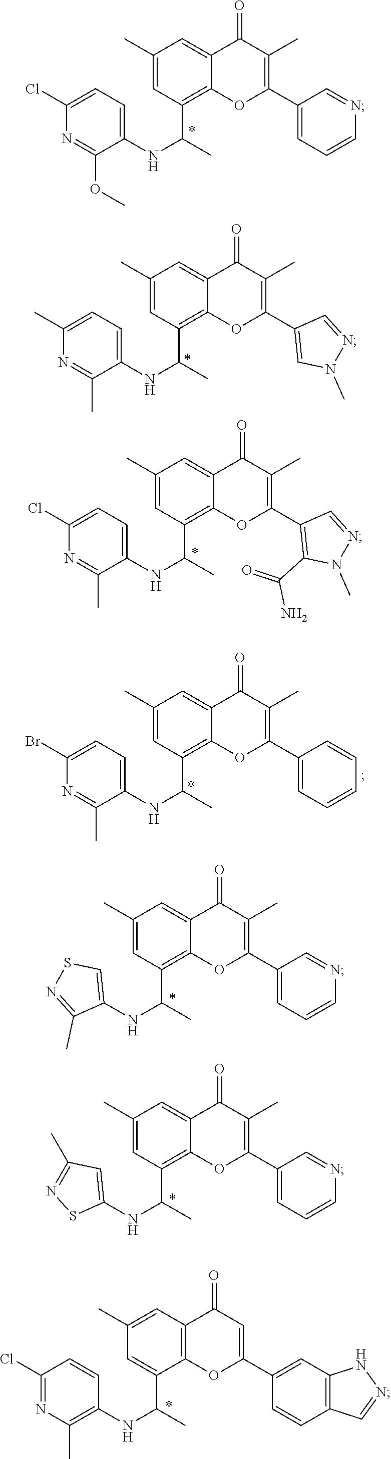

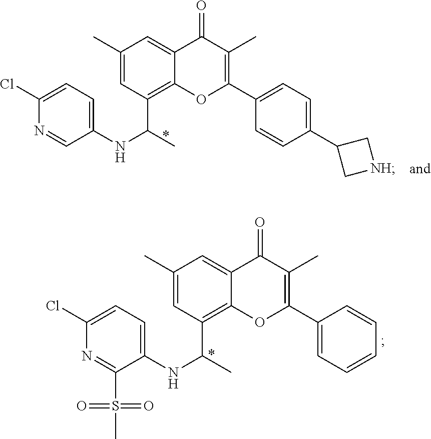 [SMILES: Cc1cc(C(C)Nc2ccc(Cl)nc2)c2oc(-c3ccc(C4CNC4)cc3)c(C)c(=O)c2c1 ; Cc1cc(C(C)Nc2ccc(Cl)nc2S(C)(=O)=O)c2oc(-c3ccccc3)c(C)c(=O)c2c1]

or a pharmaceutically acceptable salt of any of the foregoing; wherein the bond at the * position is as represented,

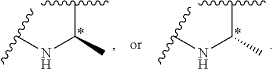

In yet a further compound of Formula (I), the compound is selected from:

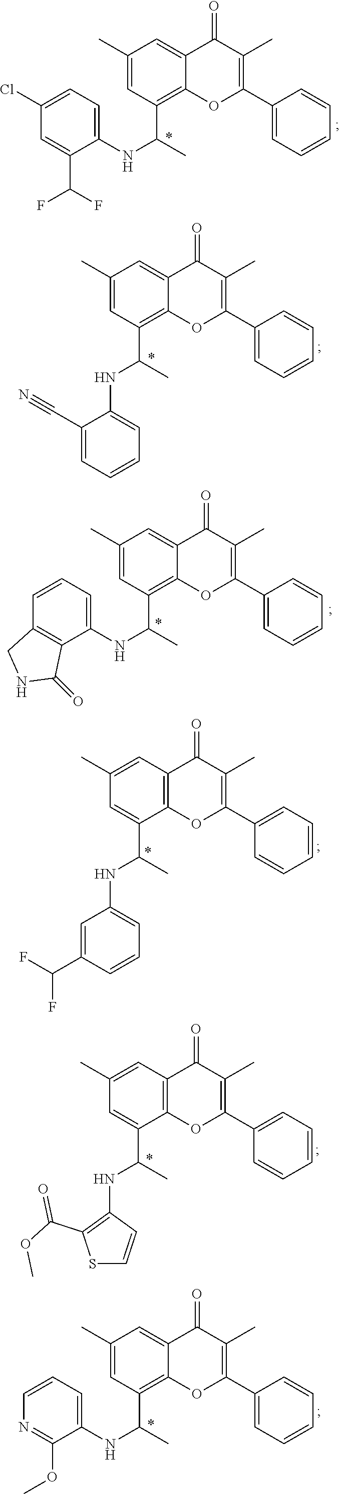 [SMILES: Cc1cc(C(C)Nc2ccc(Cl)cc2C(F)F)c2oc(-c3ccccc3)c(C)c(=O)c2c1 ; Cc1cc(C(C)Nc2ccccc2C#N)c2oc(-c3ccccc3)c(C)c(=O)c2c1 ; Cc1cc(C(C)Nc2cccc3c2C(=O)NC3)c2oc(-c3ccccc3)c(C)c(=O)c2c1 ; Cc1cc(C(C)Nc2cccc(C(F)F)c2)c2oc(-c3ccccc3)c(C)c(=O)c2c1 ; COC(=O)c1sccc1NC(C)c1cc(C)cc2c(=O)c(C)c(-c3ccccc3)oc12 ; COc1ncccc1NC(C)c1cc(C)cc2c(=O)c(C)c(-c3ccccc3)oc12]

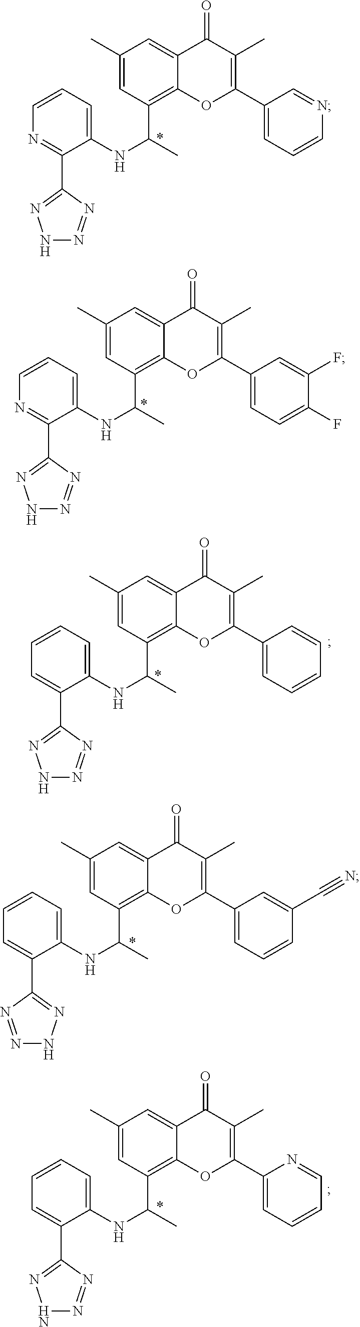 [SMILES: Cc1cc(C(C)Nc2cccnc2-c2nn[nH]n2)c2oc(-c3cccnc3)c(C)c(=O)c2c1 ; Cc1cc(C(C)Nc2cccnc2-c2nn[nH]n2)c2oc(-c3ccc(F)c(F)c3)c(C)c(=O)c2c1 ; Cc1cc(C(C)Nc2ccccc2-c2nn[nH]n2)c2oc(-c3ccccc3)c(C)c(=O)c2c1 ; Cc1cc(C(C)Nc2ccccc2-c2nn[nH]n2)c2oc(-c3cccc(C#N)c3)c(C)c(=O)c2c1 ; Cc1cc(C(C)Nc2ccccc2-c2nn[nH]n2)c2oc(-c3ccccn3)c(C)c(=O)c2c1]

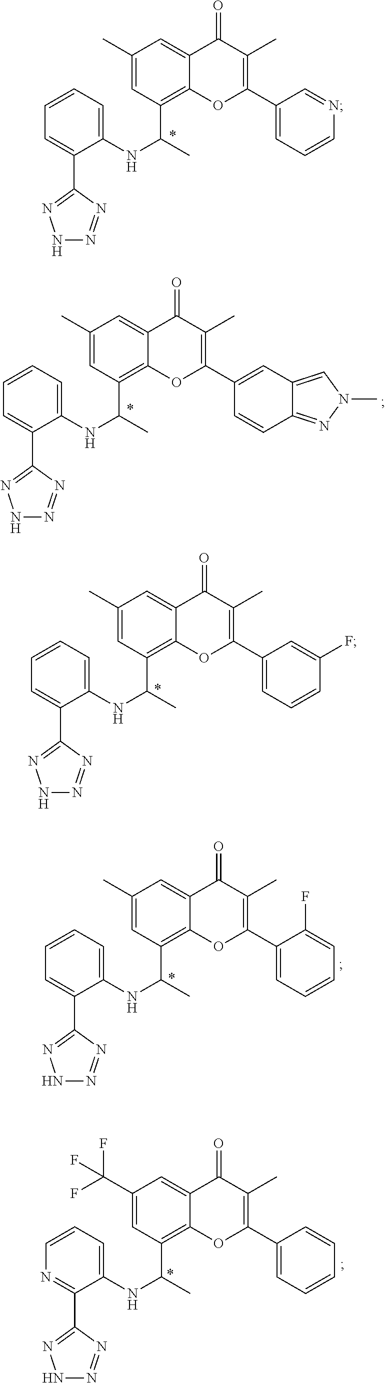 [SMILES: Cc1cc(C(C)Nc2ccccc2-c2nn[nH]n2)c2oc(-c3cccnc3)c(C)c(=O)c2c1 ; Cc1cc(C(C)Nc2ccccc2-c2nn[nH]n2)c2oc(-c3ccc4nn(C)cc4c3)c(C)c(=O)c2c1 ; Cc1cc(C(C)Nc2ccccc2-c2nn[nH]n2)c2oc(-c3cccc(F)c3)c(C)c(=O)c2c1 ; Cc1cc(C(C)Nc2ccccc2-c2nn[nH]n2)c2oc(-c3ccccc3F)c(C)c(=O)c2c1 ; Cc1c(-c2ccccc2)oc2c(C(C)Nc3cccnc3-c3nn[nH]n3)cc(C(F)(F)F)cc2c1=O]

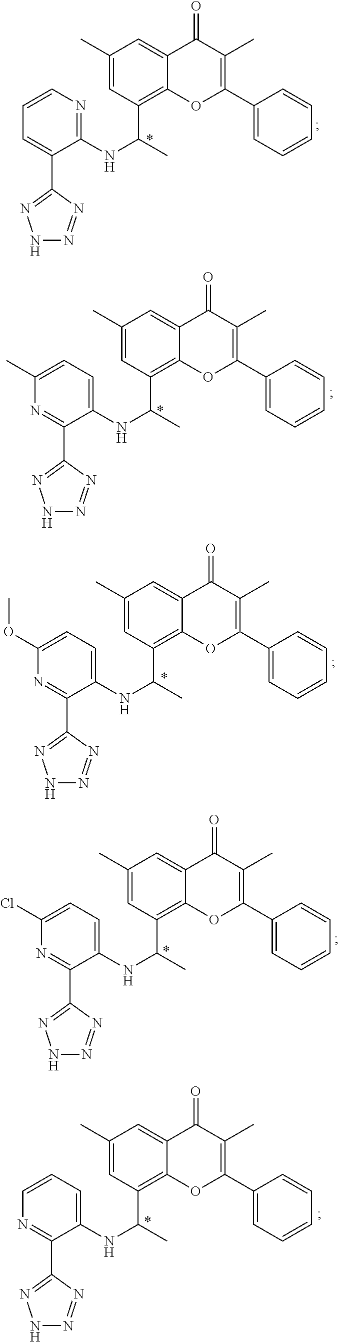 [SMILES: Cc1cc(C(C)Nc2ncccc2-c2nn[nH]n2)c2oc(-c3ccccc3)c(C)c(=O)c2c1 ; Cc1cc(C(C)Nc2ccc(C)nc2-c2nn[nH]n2)c2oc(-c3ccccc3)c(C)c(=O)c2c1 ; COc1ccc(NC(C)c2cc(C)cc3c(=O)c(C)c(-c4ccccc4)oc23)c(-c2nn[nH]n2)n1 ; Cc1cc(C(C)Nc2ccc(Cl)nc2-c2nn[nH]n2)c2oc(-c3ccccc3)c(C)c(=O)c2c1 ; Cc1cc(C(C)Nc2cccnc2-c2nn[nH]n2)c2oc(-c3ccccc3)c(C)c(=O)c2c1]

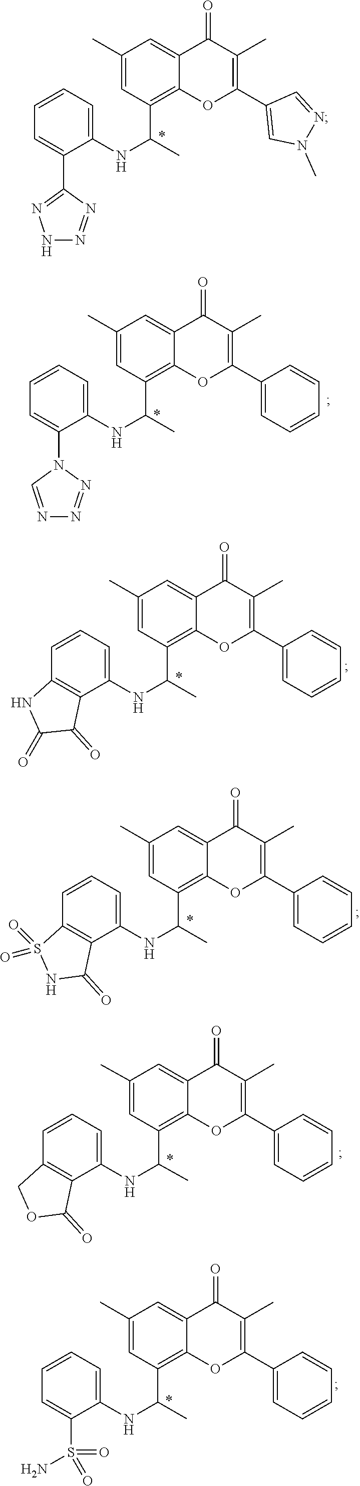 [SMILES: Cc1cc(C(C)Nc2ccccc2-c2nn[nH]n2)c2oc(-c3cnn(C)c3)c(C)c(=O)c2c1 ; Cc1cc(C(C)Nc2ccccc2-n2cnnn2)c2oc(-c3ccccc3)c(C)c(=O)c2c1 ; Cc1cc(C(C)Nc2cccc3c2C(=O)C(=O)N3)c2oc(-c3ccccc3)c(C)c(=O)c2c1 ; Cc1cc(C(C)Nc2cccc3c2C(=O)NS3(=O)=O)c2oc(-c3ccccc3)c(C)c(=O)c2c1 ; Cc1cc(C(C)Nc2cccc3c2C(=O)OC3)c2oc(-c3ccccc3)c(C)c(=O)c2c1 ; Cc1cc(C(C)Nc2ccccc2S(N)(=O)=O)c2oc(-c3ccccc3)c(C)c(=O)c2c1]

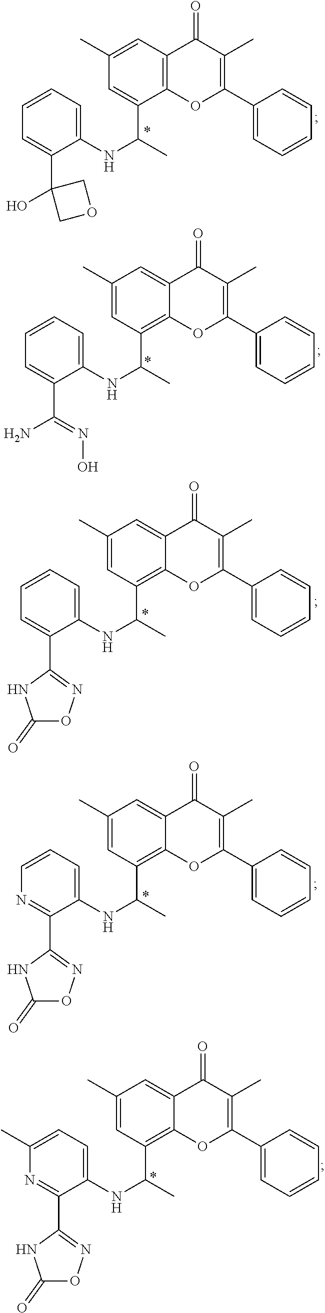 [SMILES: Cc1cc(C(C)Nc2ccccc2C2(O)COC2)c2oc(-c3ccccc3)c(C)c(=O)c2c1 ; Cc1cc(C(C)Nc2ccccc2/C(N)=N/O)c2oc(-c3ccccc3)c(C)c(=O)c2c1 ; Cc1cc(C(C)Nc2ccccc2-c2noc(=O)[nH]2)c2oc(-c3ccccc3)c(C)c(=O)c2c1 ; Cc1cc(C(C)Nc2cccnc2-c2noc(=O)[nH]2)c2oc(-c3ccccc3)c(C)c(=O)c2c1 ; Cc1cc(C(C)Nc2ccc(C)nc2-c2noc(=O)[nH]2)c2oc(-c3ccccc3)c(C)c(=O)c2c1]

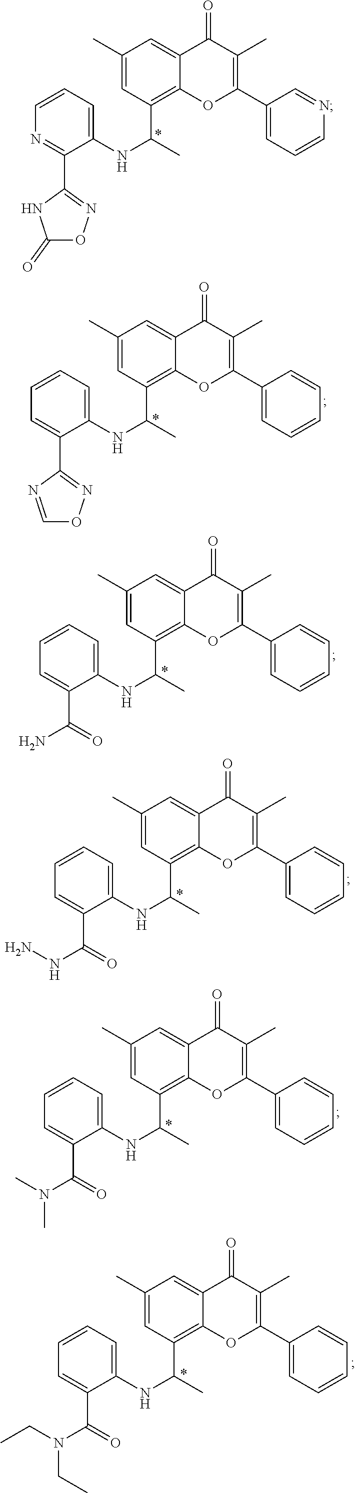 [SMILES: Cc1cc(C(C)Nc2cccnc2-c2noc(=O)[nH]2)c2oc(-c3cccnc3)c(C)c(=O)c2c1 ; Cc1cc(C(C)Nc2ccccc2-c2ncon2)c2oc(-c3ccccc3)c(C)c(=O)c2c1 ; Cc1cc(C(C)Nc2ccccc2C(N)=O)c2oc(-c3ccccc3)c(C)c(=O)c2c1 ; Cc1cc(C(C)Nc2ccccc2C(=O)NN)c2oc(-c3ccccc3)c(C)c(=O)c2c1 ; Cc1cc(C(C)Nc2ccccc2C(=O)N(C)C)c2oc(-c3ccccc3)c(C)c(=O)c2c1 ; CCN(CC)C(=O)c1ccccc1NC(C)c1cc(C)cc2c(=O)c(C)c(-c3ccccc3)oc12]

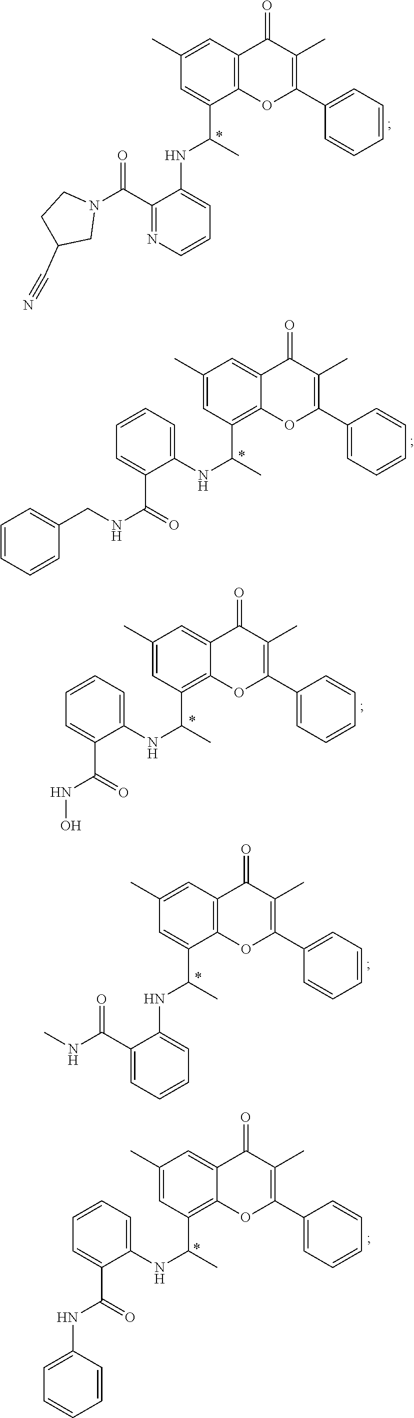 [SMILES: Cc1cc(C(C)Nc2cccnc2C(=O)N2CCC(C#N)C2)c2oc(-c3ccccc3)c(C)c(=O)c2c1 ; Cc1cc(C(C)Nc2ccccc2C(=O)NCc2ccccc2)c2oc(-c3ccccc3)c(C)c(=O)c2c1 ; Cc1cc(C(C)Nc2ccccc2C(=O)NO)c2oc(-c3ccccc3)c(C)c(=O)c2c1 ; CNC(=O)c1ccccc1NC(C)c1cc(C)cc2c(=O)c(C)c(-c3ccccc3)oc12 ; Cc1cc(C(C)Nc2ccccc2C(=O)Nc2ccccc2)c2oc(-c3ccccc3)c(C)c(=O)c2c1]

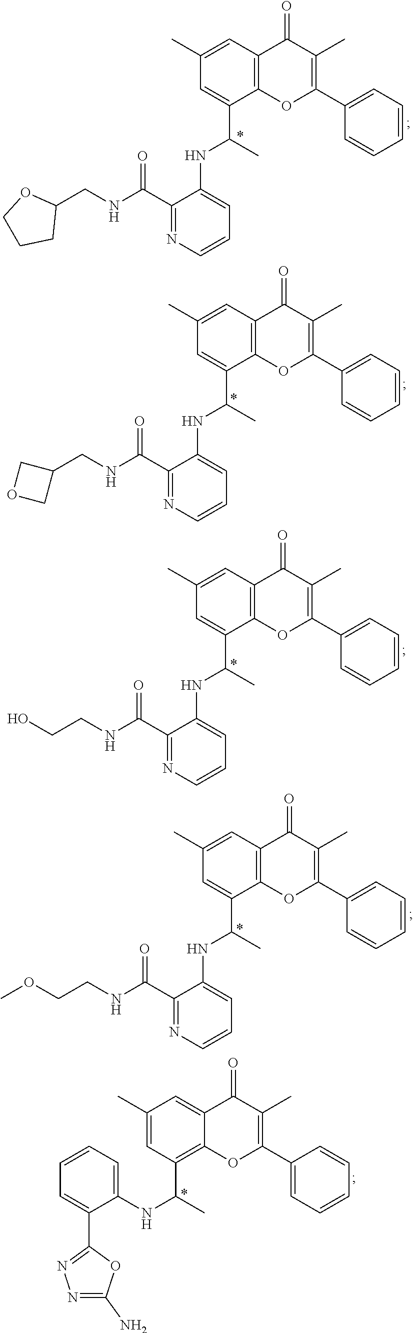

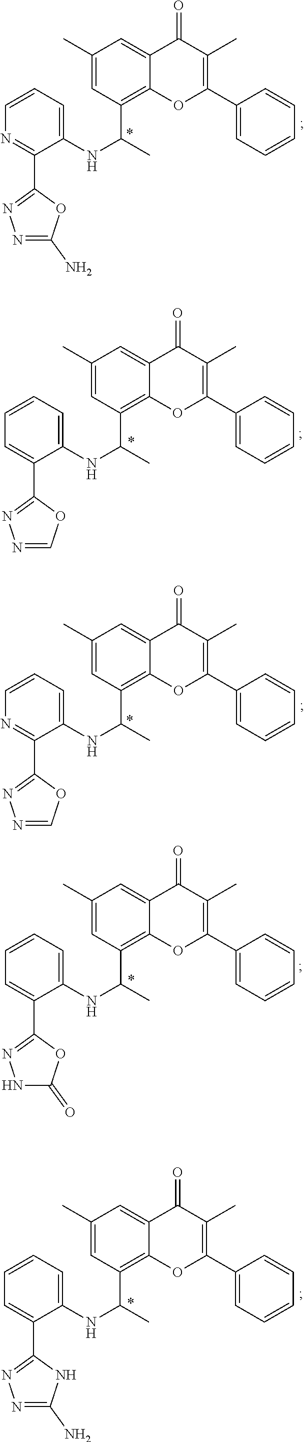 [SMILES: Cc1cc(C(C)Nc2cccnc2-c2nnc(N)o2)c2oc(-c3ccccc3)c(C)c(=O)c2c1 ; Cc1cc(C(C)Nc2ccccc2-c2nnco2)c2oc(-c3ccccc3)c(C)c(=O)c2c1 ; Cc1cc(C(C)Nc2cccnc2-c2nnco2)c2oc(-c3ccccc3)c(C)c(=O)c2c1 ; Cc1cc(C(C)Nc2ccccc2-c2n[nH]c(=O)o2)c2oc(-c3ccccc3)c(C)c(=O)c2c1 ; Cc1cc(C(C)Nc2ccccc2-c2nnc(N)[nH]2)c2oc(-c3ccccc3)c(C)c(=O)c2c1]

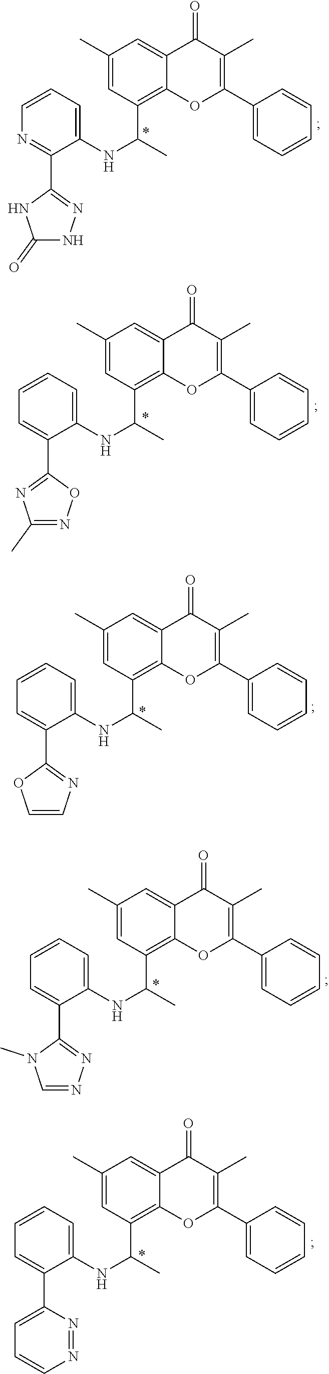 [SMILES: Cc1cc(C(C)Nc2cccnc2-c2n[nH]c(=O)[nH]2)c2oc(-c3ccccc3)c(C)c(=O)c2c1 ; Cc1cc(C(C)Nc2ccccc2-c2nc(C)no2)c2oc(-c3ccccc3)c(C)c(=O)c2c1 ; Cc1cc(C(C)Nc2ccccc2-c2ncco2)c2oc(-c3ccccc3)c(C)c(=O)c2c1 ; Cc1cc(C(C)Nc2ccccc2-c2nncn2C)c2oc(-c3ccccc3)c(C)c(=O)c2c1 ; Cc1cc(C(C)Nc2ccccc2-c2cccnn2)c2oc(-c3ccccc3)c(C)c(=O)c2c1]

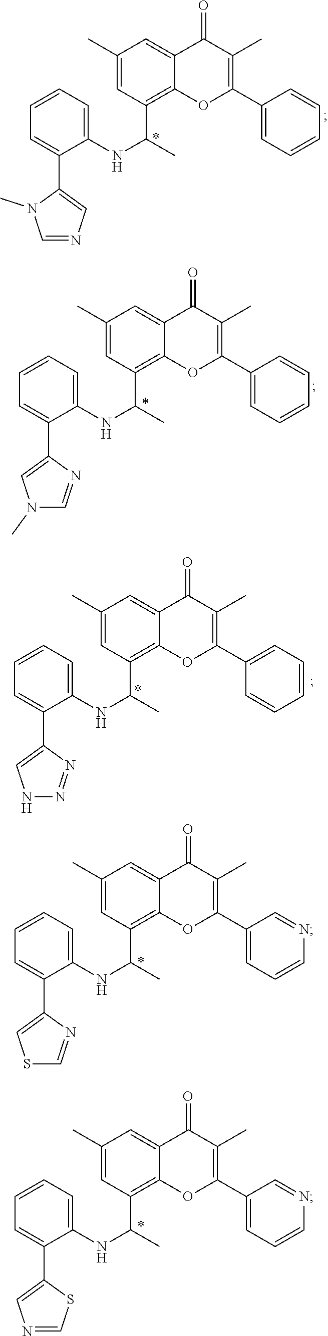 [SMILES: Cc1cc(C(C)Nc2ccccc2-c2cncn2C)c2oc(-c3ccccc3)c(C)c(=O)c2c1 ; Cc1cc(C(C)Nc2ccccc2-c2cn(C)cn2)c2oc(-c3ccccc3)c(C)c(=O)c2c1 ; Cc1cc(C(C)Nc2ccccc2-c2c[nH]nn2)c2oc(-c3ccccc3)c(C)c(=O)c2c1 ; Cc1cc(C(C)Nc2ccccc2-c2cscn2)c2oc(-c3cccnc3)c(C)c(=O)c2c1 ; Cc1cc(C(C)Nc2ccccc2-c2cncs2)c2oc(-c3cccnc3)c(C)c(=O)c2c1]

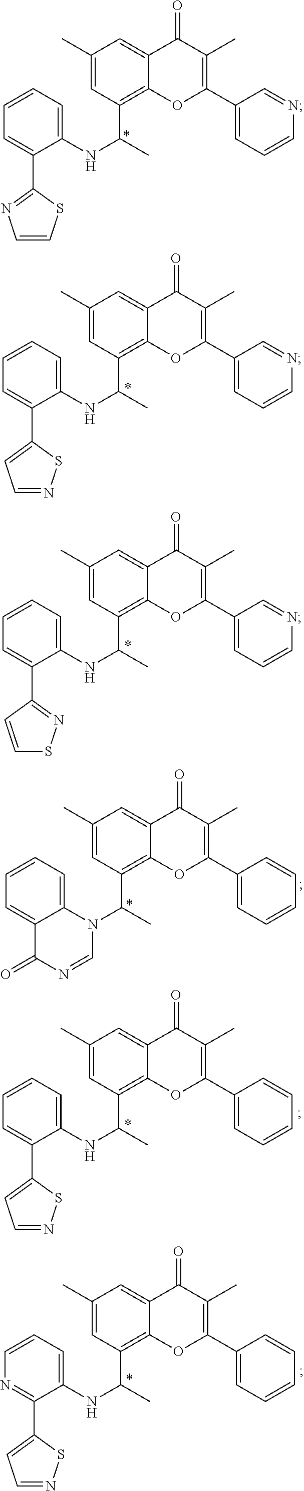 [SMILES: Cc1cc(C(C)Nc2ccccc2-c2nccs2)c2oc(-c3cccnc3)c(C)c(=O)c2c1 ; Cc1cc(C(C)Nc2ccccc2-c2ccns2)c2oc(-c3cccnc3)c(C)c(=O)c2c1 ; Cc1cc(C(C)Nc2ccccc2-c2ccsn2)c2oc(-c3cccnc3)c(C)c(=O)c2c1 ; Cc1cc(C(C)n2cnc(=O)c3ccccc32)c2oc(-c3ccccc3)c(C)c(=O)c2c1 ; Cc1cc(C(C)Nc2ccccc2-c2ccns2)c2oc(-c3ccccc3)c(C)c(=O)c2c1 ; Cc1cc(C(C)Nc2cccnc2-c2ccns2)c2oc(-c3ccccc3)c(C)c(=O)c2c1]

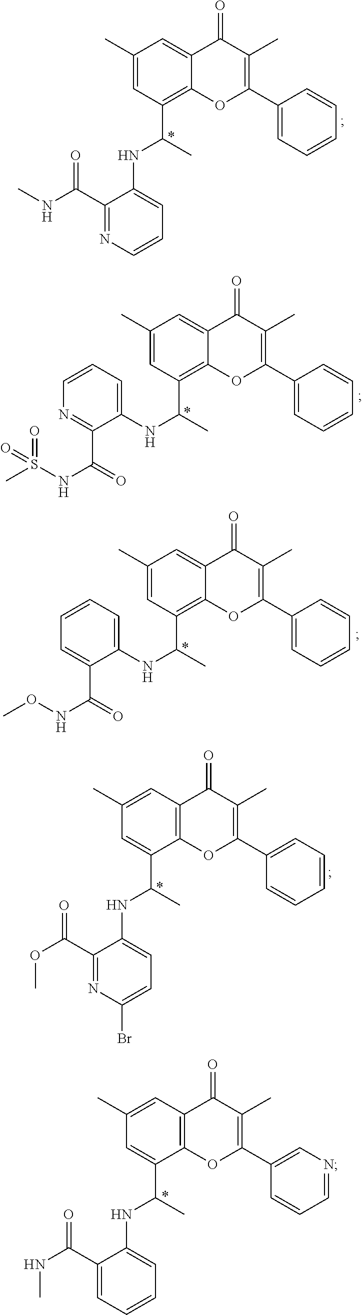 [SMILES: CNC(=O)c1ncccc1NC(C)c1cc(C)cc2c(=O)c(C)c(-c3ccccc3)oc12 ; Cc1cc(C(C)Nc2cccnc2C(=O)NS(C)(=O)=O)c2oc(-c3ccccc3)c(C)c(=O)c2c1 ; CONC(=O)c1ccccc1NC(C)c1cc(C)cc2c(=O)c(C)c(-c3ccccc3)oc12 ; COC(=O)c1nc(Br)ccc1NC(C)c1cc(C)cc2c(=O)c(C)c(-c3ccccc3)oc12 ; CNC(=O)c1ccccc1NC(C)c1cc(C)cc2c(=O)c(C)c(-c3cccnc3)oc12]

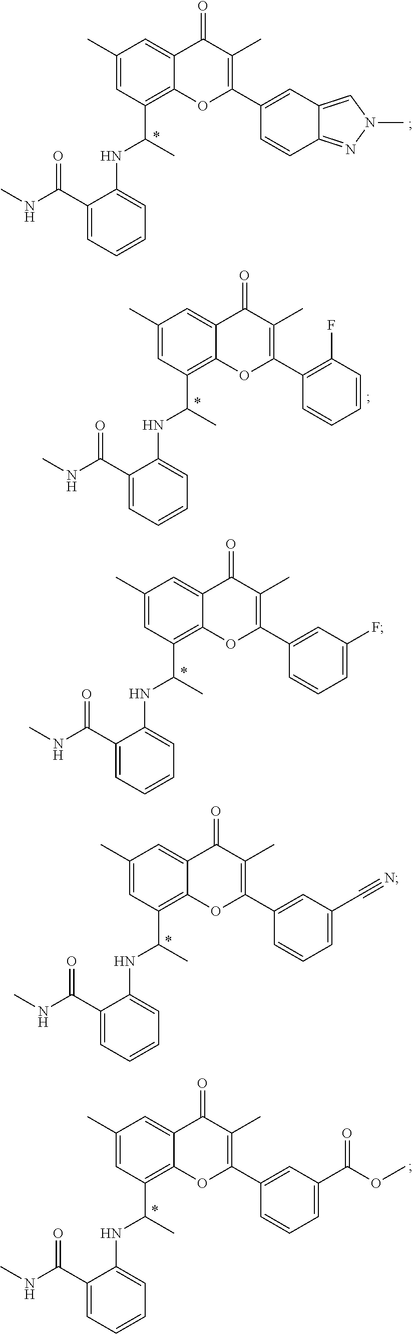

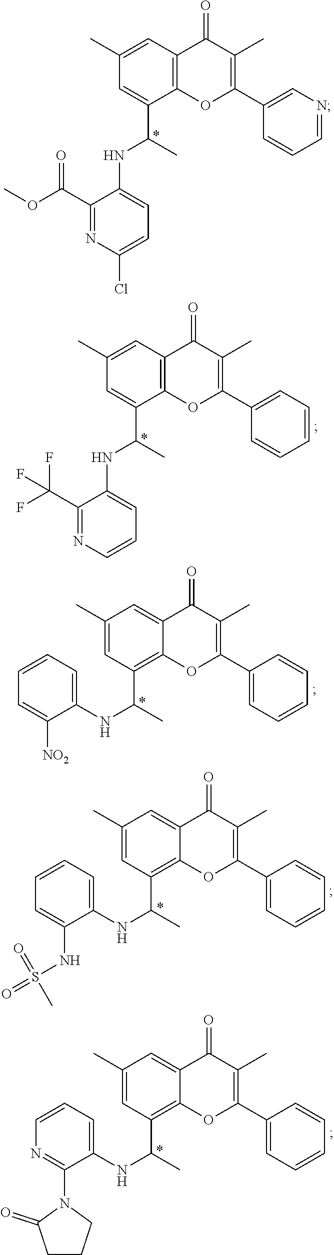 [SMILES: COC(=O)c1nc(Cl)ccc1NC(C)c1cc(C)cc2c(=O)c(C)c(-c3cccnc3)oc12 ; Cc1cc(C(C)Nc2cccnc2C(F)(F)F)c2oc(-c3ccccc3)c(C)c(=O)c2c1 ; Cc1cc(C(C)Nc2ccccc2[N+](=O)[O-])c2oc(-c3ccccc3)c(C)c(=O)c2c1 ; Cc1cc(C(C)Nc2ccccc2NS(C)(=O)=O)c2oc(-c3ccccc3)c(C)c(=O)c2c1 ; Cc1cc(C(C)Nc2cccnc2N2CCCC2=O)c2oc(-c3ccccc3)c(C)c(=O)c2c1]

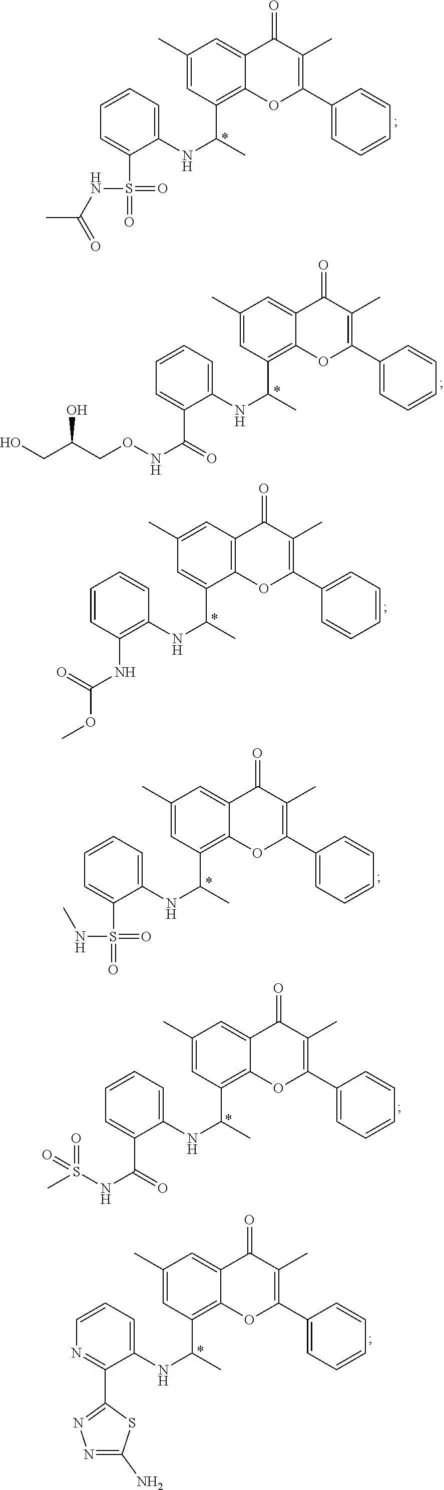 [SMILES: CC(=O)NS(=O)(=O)c1ccccc1NC(C)c1cc(C)cc2c(=O)c(C)c(-c3ccccc3)oc12 ; Cc1cc(C(C)Nc2ccccc2C(=O)NOC[C@H](O)CO)c2oc(-c3ccccc3)c(C)c(=O)c2c1 ; COC(=O)Nc1ccccc1NC(C)c1cc(C)cc2c(=O)c(C)c(-c3ccccc3)oc12 ; CNS(=O)(=O)c1ccccc1NC(C)c1cc(C)cc2c(=O)c(C)c(-c3ccccc3)oc12 ; Cc1cc(C(C)Nc2ccccc2C(=O)NS(C)(=O)=O)c2oc(-c3ccccc3)c(C)c(=O)c2c1 ; Cc1cc(C(C)Nc2cccnc2-c2nnc(N)s2)c2oc(-c3ccccc3)c(C)c(=O)c2c1]

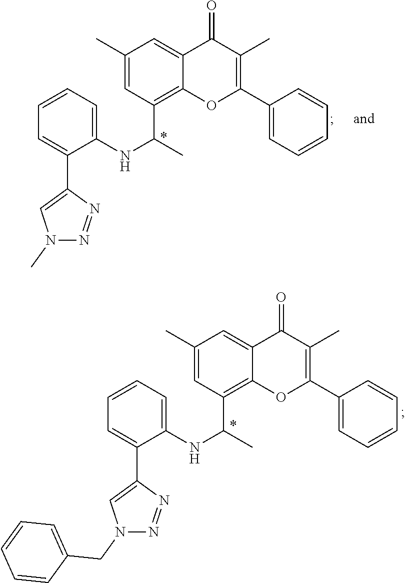 [SMILES: Cc1cc(C(C)Nc2ccccc2-c2cn(C)nn2)c2oc(-c3ccccc3)c(C)c(=O)c2c1 ; Cc1cc(C(C)Nc2ccccc2-c2cn(Cc3ccccc3)nn2)c2oc(-c3ccccc3)c(C)c(=O)c2c1]

or a pharmaceutically acceptable salt of any of the foregoing;
wherein the bond at the * position is as represented,

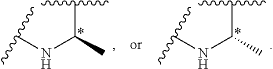

In yet a further compound of Formula (I), the compound is selected from

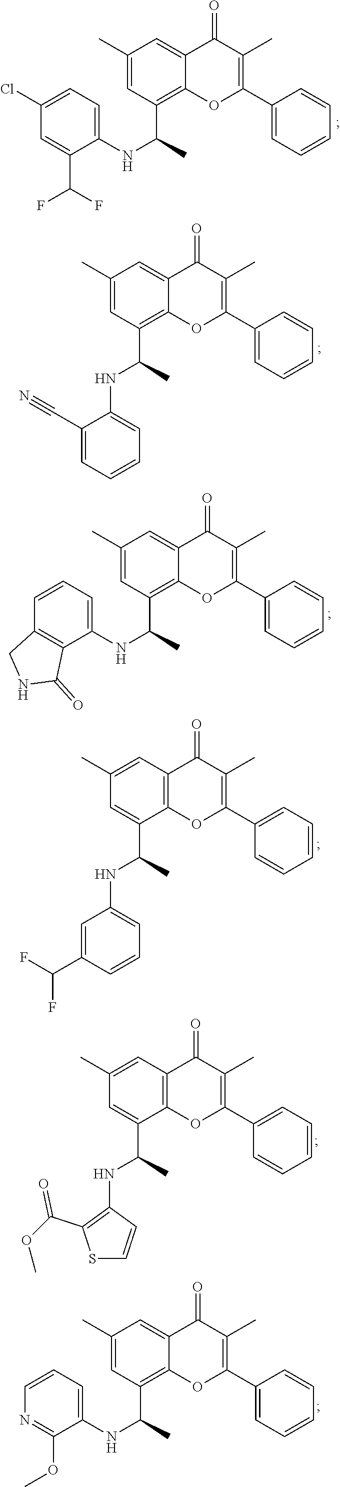 [SMILES: Cc1cc([C@@H](C)Nc2ccc(Cl)cc2C(F)F)c2oc(-c3ccccc3)c(C)c(=O)c2c1 ; Cc1cc([C@@H](C)Nc2ccccc2C#N)c2oc(-c3ccccc3)c(C)c(=O)c2c1 ; Cc1cc([C@@H](C)Nc2cccc3c2C(=O)NC3)c2oc(-c3ccccc3)c(C)c(=O)c2c1 ; Cc1cc([C@@H](C)Nc2cccc(C(F)F)c2)c2oc(-c3ccccc3)c(C)c(=O)c2c1 ; COC(=O)c1sccc1N[C@H](C)c1cc(C)cc2c(=O)c(C)c(-c3ccccc3)oc12 ; COc1ncccc1N[C@H](C)c1cc(C)cc2c(=O)c(C)c(-c3ccccc3)oc12]

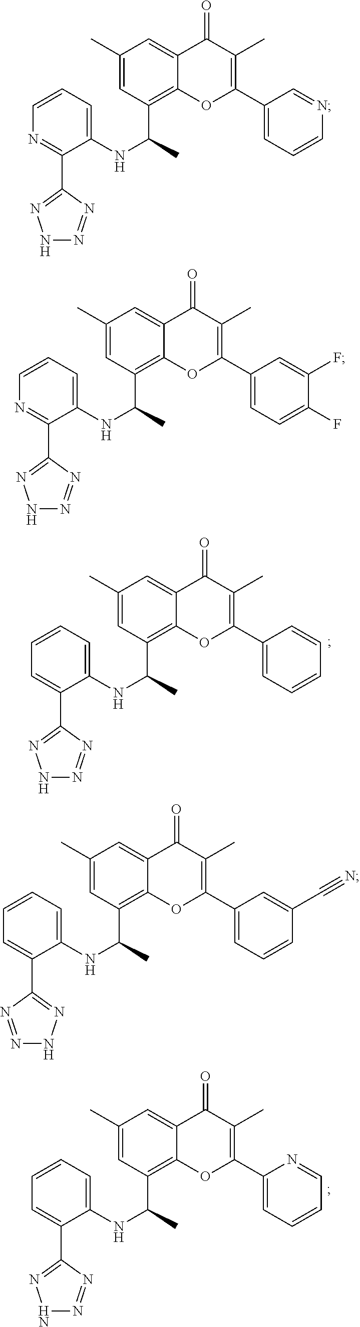 [SMILES: Cc1cc([C@@H](C)Nc2cccnc2-c2nn[nH]n2)c2oc(-c3cccnc3)c(C)c(=O)c2c1 ; Cc1cc([C@@H](C)Nc2cccnc2-c2nn[nH]n2)c2oc(-c3ccc(F)c(F)c3)c(C)c(=O)c2c1 ; Cc1cc([C@@H](C)Nc2ccccc2-c2nn[nH]n2)c2oc(-c3ccccc3)c(C)c(=O)c2c1 ; Cc1cc([C@@H](C)Nc2ccccc2-c2nn[nH]n2)c2oc(-c3cccc(C#N)c3)c(C)c(=O)c2c1 ; Cc1cc([C@@H](C)Nc2ccccc2-c2nn[nH]n2)c2oc(-c3ccccn3)c(C)c(=O)c2c1]

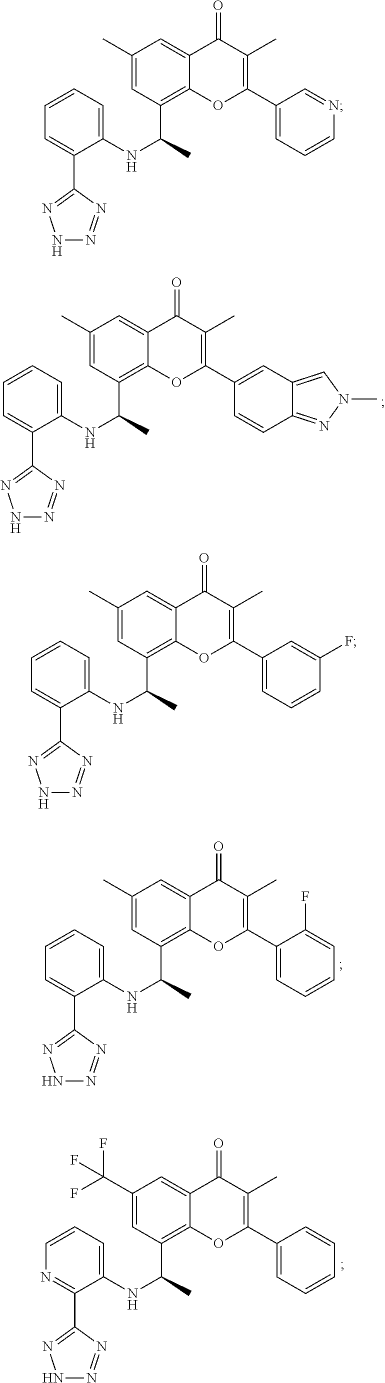 [SMILES: Cc1cc([C@@H](C)Nc2ccccc2-c2nn[nH]n2)c2oc(-c3cccnc3)c(C)c(=O)c2c1 ; Cc1cc([C@@H](C)Nc2ccccc2-c2nn[nH]n2)c2oc(-c3ccc4nn(C)cc4c3)c(C)c(=O)c2c1 ; Cc1cc([C@@H](C)Nc2ccccc2-c2nn[nH]n2)c2oc(-c3cccc(F)c3)c(C)c(=O)c2c1 ; Cc1cc([C@@H](C)Nc2ccccc2-c2nn[nH]n2)c2oc(-c3ccccc3F)c(C)c(=O)c2c1 ; Cc1c(-c2ccccc2)oc2c([C@@H](C)Nc3cccnc3-c3nn[nH]n3)cc(C(F)(F)F)cc2c1=O]

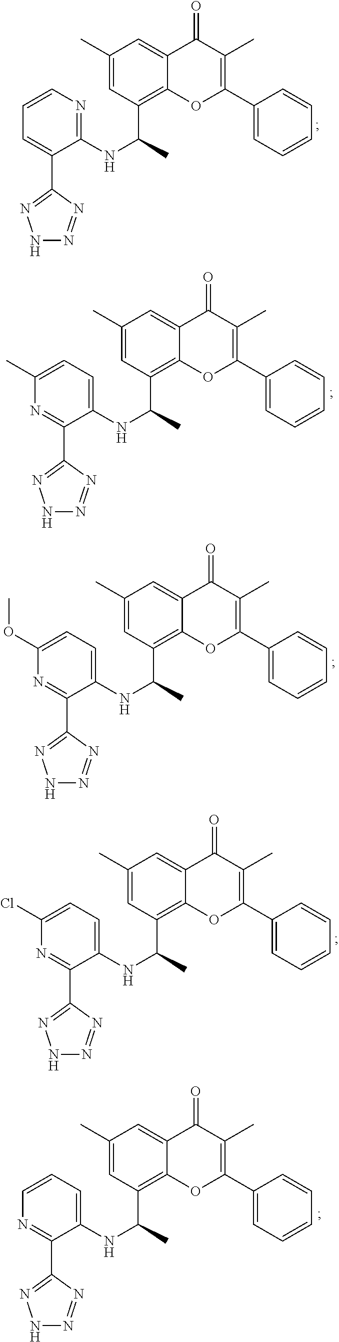 [SMILES: Cc1cc([C@@H](C)Nc2ncccc2-c2nn[nH]n2)c2oc(-c3ccccc3)c(C)c(=O)c2c1 ; Cc1cc([C@@H](C)Nc2ccc(C)nc2-c2nn[nH]n2)c2oc(-c3ccccc3)c(C)c(=O)c2c1 ; COc1ccc(N[C@H](C)c2cc(C)cc3c(=O)c(C)c(-c4ccccc4)oc23)c(-c2nn[nH]n2)n1 ; Cc1cc([C@@H](C)Nc2ccc(Cl)nc2-c2nn[nH]n2)c2oc(-c3ccccc3)c(C)c(=O)c2c1 ; Cc1cc([C@@H](C)Nc2cccnc2-c2nn[nH]n2)c2oc(-c3ccccc3)c(C)c(=O)c2c1]

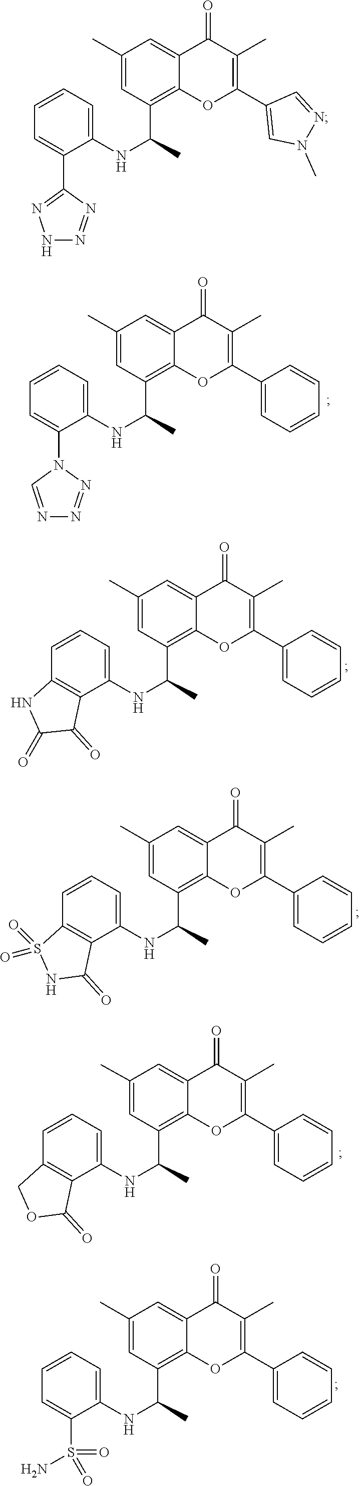 [SMILES: Cc1cc([C@@H](C)Nc2ccccc2-c2nn[nH]n2)c2oc(-c3cnn(C)c3)c(C)c(=O)c2c1 ; Cc1cc([C@@H](C)Nc2ccccc2-n2cnnn2)c2oc(-c3ccccc3)c(C)c(=O)c2c1 ; Cc1cc([C@@H](C)Nc2cccc3c2C(=O)C(=O)N3)c2oc(-c3ccccc3)c(C)c(=O)c2c1 ; Cc1cc([C@@H](C)Nc2cccc3c2C(=O)NS3(=O)=O)c2oc(-c3ccccc3)c(C)c(=O)c2c1 ; Cc1cc([C@@H](C)Nc2cccc3c2C(=O)OC3)c2oc(-c3ccccc3)c(C)c(=O)c2c1 ; Cc1cc([C@@H](C)Nc2ccccc2S(N)(=O)=O)c2oc(-c3ccccc3)c(C)c(=O)c2c1]

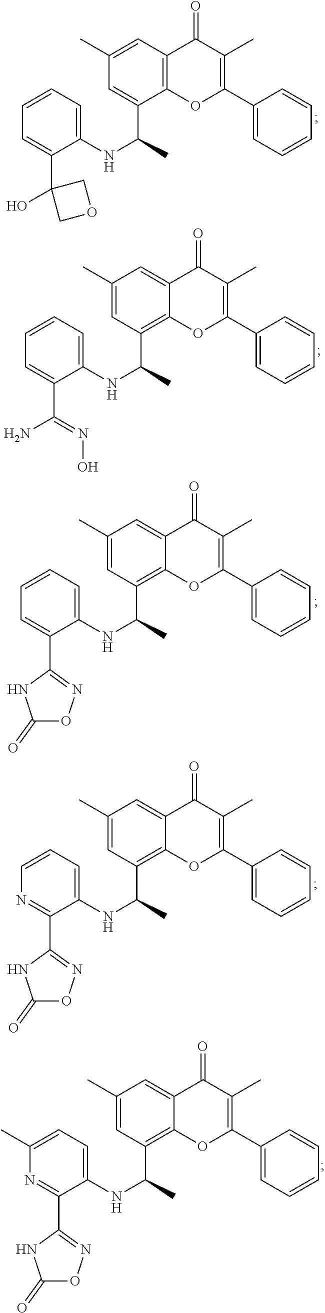 [SMILES: Cc1cc([C@@H](C)Nc2ccccc2C2(O)COC2)c2oc(-c3ccccc3)c(C)c(=O)c2c1 ; Cc1cc([C@@H](C)Nc2ccccc2/C(N)=N/O)c2oc(-c3ccccc3)c(C)c(=O)c2c1 ; Cc1cc([C@@H](C)Nc2ccccc2-c2noc(=O)[nH]2)c2oc(-c3ccccc3)c(C)c(=O)c2c1 ; Cc1cc([C@@H](C)Nc2cccnc2-c2noc(=O)[nH]2)c2oc(-c3ccccc3)c(C)c(=O)c2c1 ; Cc1cc([C@@H](C)Nc2ccc(C)nc2-c2noc(=O)[nH]2)c2oc(-c3ccccc3)c(C)c(=O)c2c1]

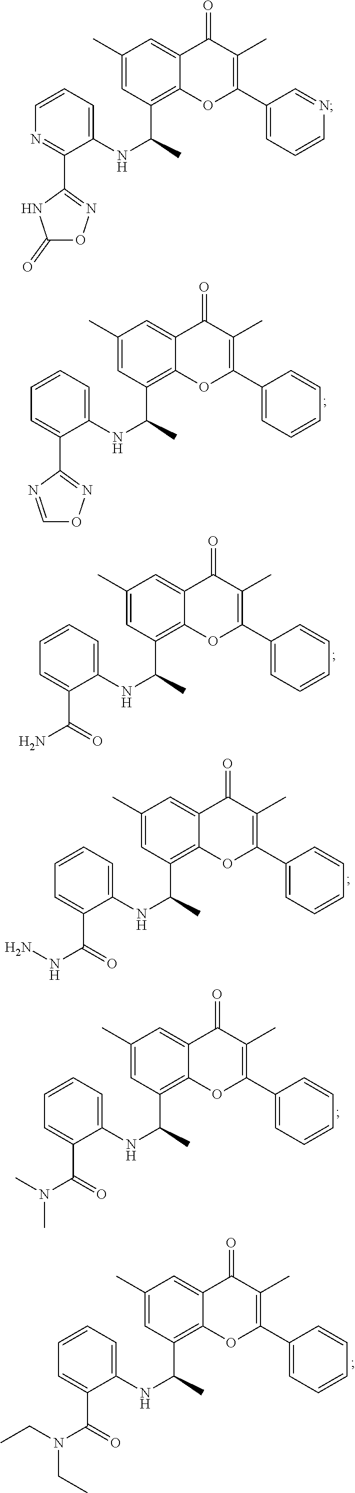 [SMILES: Cc1cc([C@@H](C)Nc2cccnc2-c2noc(=O)[nH]2)c2oc(-c3cccnc3)c(C)c(=O)c2c1 ; Cc1cc([C@@H](C)Nc2ccccc2-c2ncon2)c2oc(-c3ccccc3)c(C)c(=O)c2c1 ; Cc1cc([C@@H](C)Nc2ccccc2C(N)=O)c2oc(-c3ccccc3)c(C)c(=O)c2c1 ; Cc1cc([C@@H](C)Nc2ccccc2C(=O)NN)c2oc(-c3ccccc3)c(C)c(=O)c2c1 ; Cc1cc([C@@H](C)Nc2ccccc2C(=O)N(C)C)c2oc(-c3ccccc3)c(C)c(=O)c2c1 ; CCN(CC)C(=O)c1ccccc1N[C@H](C)c1cc(C)cc2c(=O)c(C)c(-c3ccccc3)oc12]

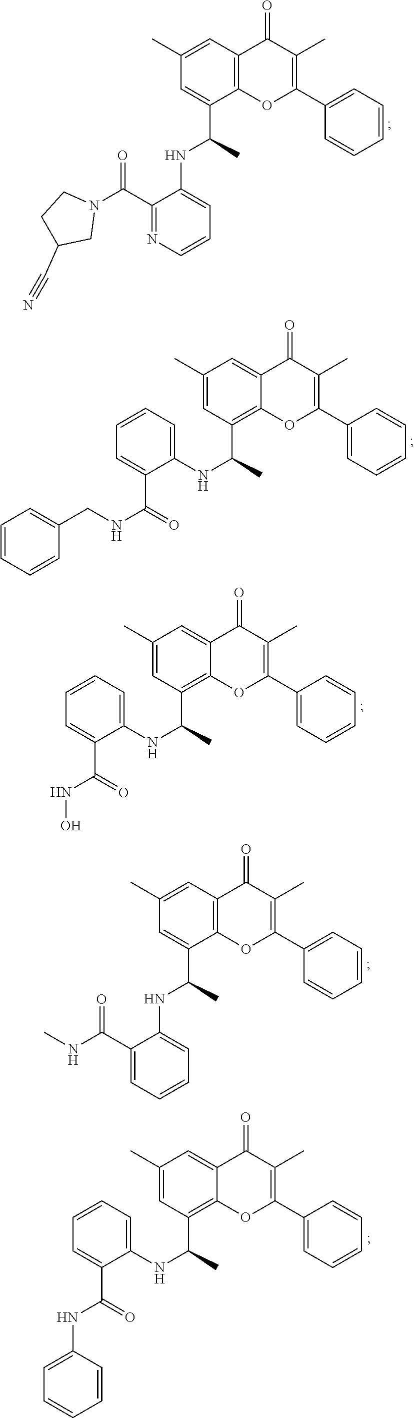 [SMILES: Cc1cc([C@@H](C)Nc2cccnc2C(=O)N2CCC(C#N)C2)c2oc(-c3ccccc3)c(C)c(=O)c2c1 ; Cc1cc([C@@H](C)Nc2ccccc2C(=O)NCc2ccccc2)c2oc(-c3ccccc3)c(C)c(=O)c2c1 ; Cc1cc([C@@H](C)Nc2ccccc2C(=O)NO)c2oc(-c3ccccc3)c(C)c(=O)c2c1 ; CNC(=O)c1ccccc1N[C@H](C)c1cc(C)cc2c(=O)c(C)c(-c3ccccc3)oc12 ; Cc1cc([C@@H](C)Nc2ccccc2C(=O)Nc2ccccc2)c2oc(-c3ccccc3)c(C)c(=O)c2c1]

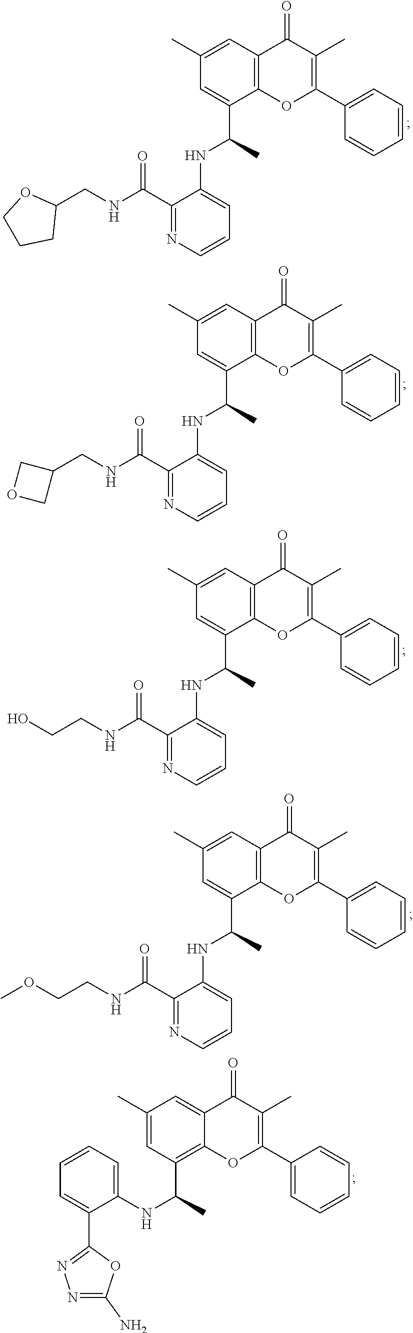

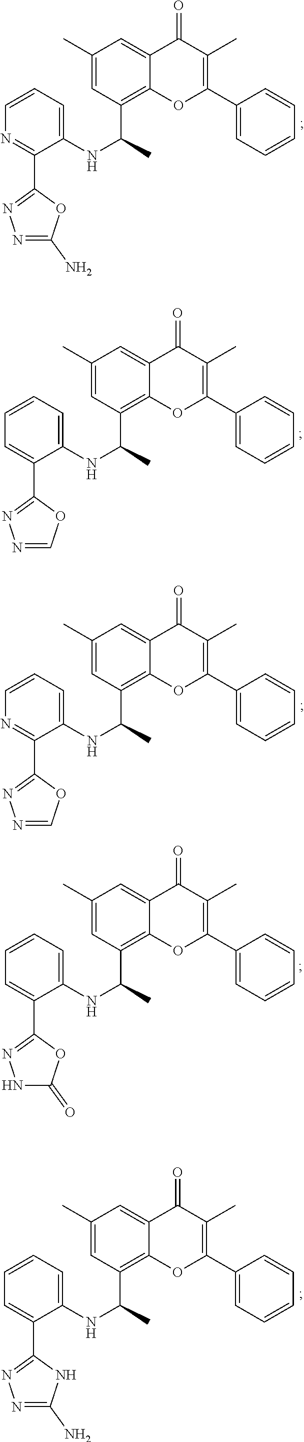 [SMILES: Cc1cc([C@@H](C)Nc2cccnc2-c2nnc(N)o2)c2oc(-c3ccccc3)c(C)c(=O)c2c1 ; Cc1cc([C@@H](C)Nc2ccccc2-c2nnco2)c2oc(-c3ccccc3)c(C)c(=O)c2c1 ; Cc1cc([C@@H](C)Nc2cccnc2-c2nnco2)c2oc(-c3ccccc3)c(C)c(=O)c2c1 ; Cc1cc([C@@H](C)Nc2ccccc2-c2n[nH]c(=O)o2)c2oc(-c3ccccc3)c(C)c(=O)c2c1 ; Cc1cc([C@@H](C)Nc2ccccc2-c2nnc(N)[nH]2)c2oc(-c3ccccc3)c(C)c(=O)c2c1]

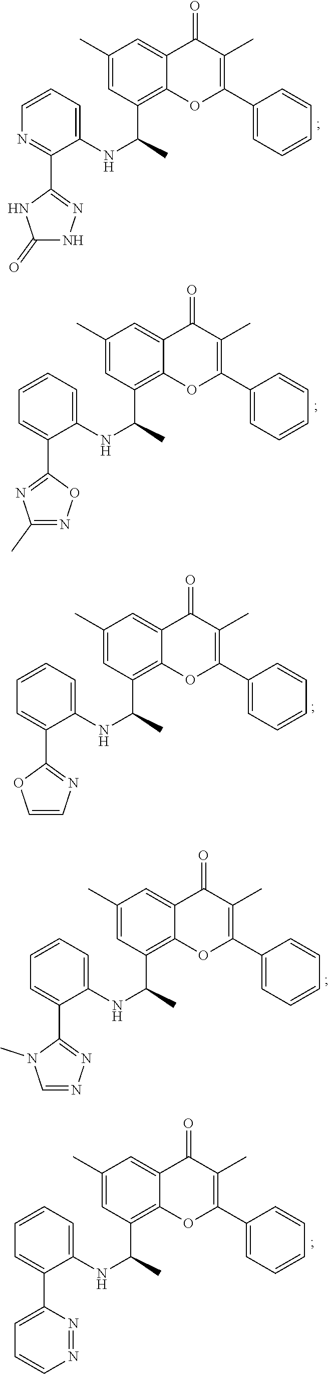 [SMILES: Cc1cc([C@@H](C)Nc2cccnc2-c2n[nH]c(=O)[nH]2)c2oc(-c3ccccc3)c(C)c(=O)c2c1 ; Cc1cc([C@@H](C)Nc2ccccc2-c2nc(C)no2)c2oc(-c3ccccc3)c(C)c(=O)c2c1 ; Cc1cc([C@@H](C)Nc2ccccc2-c2ncco2)c2oc(-c3ccccc3)c(C)c(=O)c2c1 ; Cc1cc([C@@H](C)Nc2ccccc2-c2nncn2C)c2oc(-c3ccccc3)c(C)c(=O)c2c1 ; Cc1cc([C@@H](C)Nc2ccccc2-c2cccnn2)c2oc(-c3ccccc3)c(C)c(=O)c2c1]

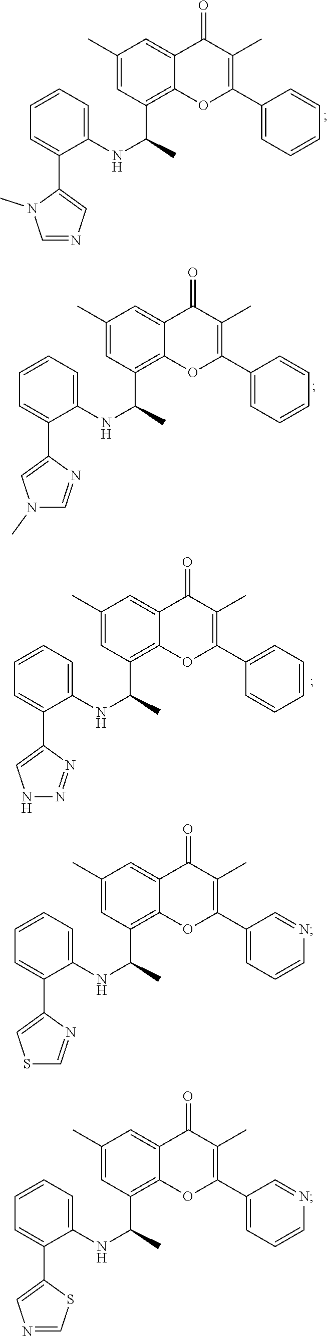 [SMILES: Cc1cc([C@@H](C)Nc2ccccc2-c2cncn2C)c2oc(-c3ccccc3)c(C)c(=O)c2c1 ; Cc1cc([C@@H](C)Nc2ccccc2-c2cn(C)cn2)c2oc(-c3ccccc3)c(C)c(=O)c2c1 ; Cc1cc([C@@H](C)Nc2ccccc2-c2c[nH]nn2)c2oc(-c3ccccc3)c(C)c(=O)c2c1 ; Cc1cc([C@@H](C)Nc2ccccc2-c2cscn2)c2oc(-c3cccnc3)c(C)c(=O)c2c1 ; Cc1cc([C@@H](C)Nc2ccccc2-c2cncs2)c2oc(-c3cccnc3)c(C)c(=O)c2c1]

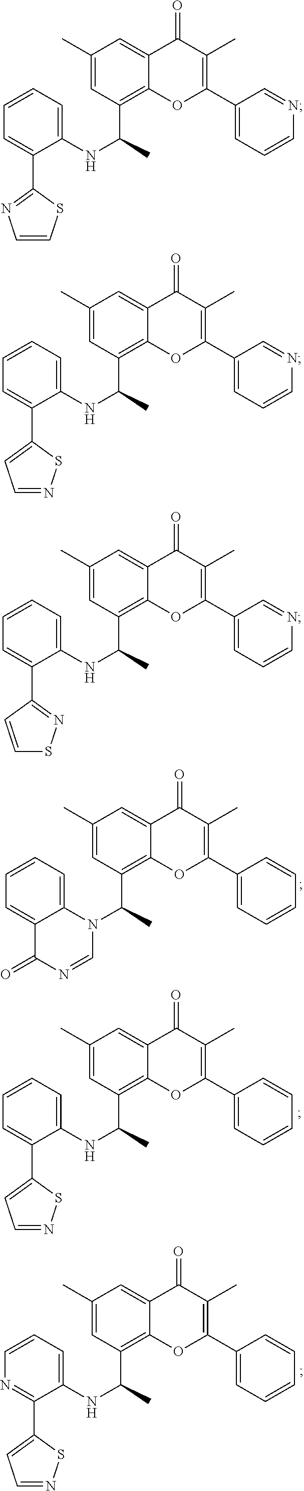 [SMILES: Cc1cc([C@@H](C)Nc2ccccc2-c2nccs2)c2oc(-c3cccnc3)c(C)c(=O)c2c1 ; Cc1cc([C@@H](C)Nc2ccccc2-c2ccns2)c2oc(-c3cccnc3)c(C)c(=O)c2c1 ; Cc1cc([C@@H](C)Nc2ccccc2-c2ccsn2)c2oc(-c3cccnc3)c(C)c(=O)c2c1 ; Cc1cc([C@@H](C)n2cnc(=O)c3ccccc32)c2oc(-c3ccccc3)c(C)c(=O)c2c1 ; Cc1cc([C@@H](C)Nc2ccccc2-c2ccns2)c2oc(-c3ccccc3)c(C)c(=O)c2c1 ; Cc1cc([C@@H](C)Nc2cccnc2-c2ccns2)c2oc(-c3ccccc3)c(C)c(=O)c2c1]

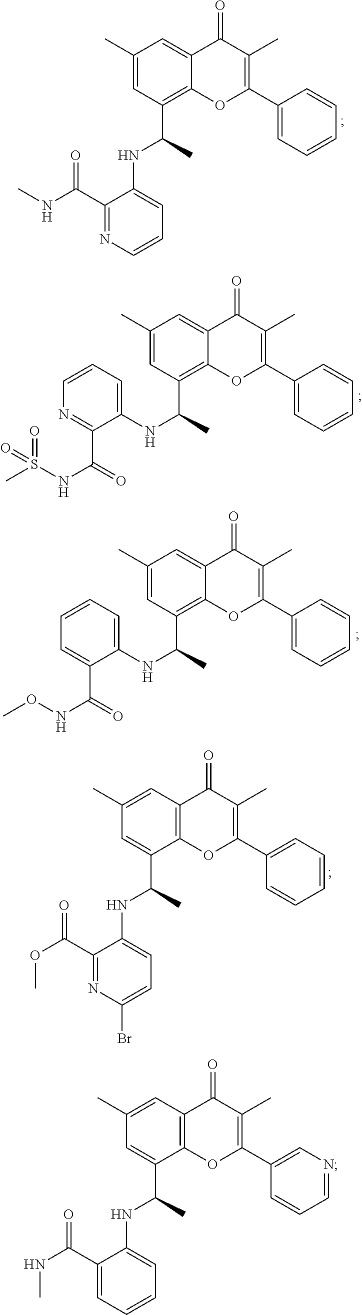 [SMILES: CNC(=O)c1ncccc1N[C@H](C)c1cc(C)cc2c(=O)c(C)c(-c3ccccc3)oc12 ; Cc1cc([C@@H](C)Nc2cccnc2C(=O)NS(C)(=O)=O)c2oc(-c3ccccc3)c(C)c(=O)c2c1 ; CONC(=O)c1ccccc1N[C@H](C)c1cc(C)cc2c(=O)c(C)c(-c3ccccc3)oc12 ; COC(=O)c1nc(Br)ccc1N[C@H](C)c1cc(C)cc2c(=O)c(C)c(-c3ccccc3)oc12 ; CNC(=O)c1ccccc1N[C@H](C)c1cc(C)cc2c(=O)c(C)c(-c3cccnc3)oc12]

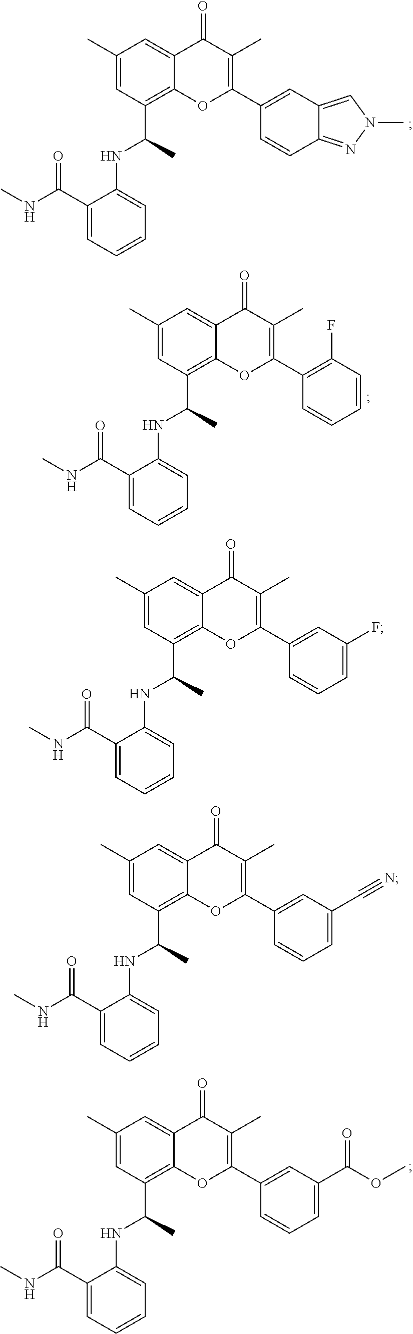

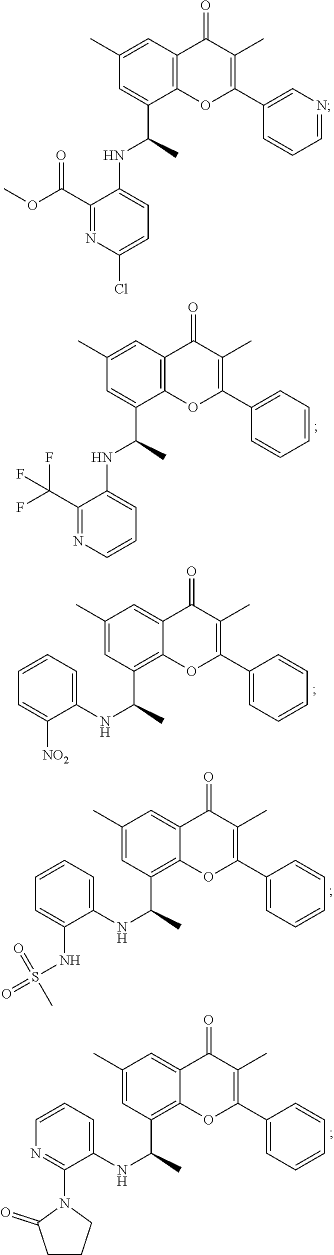 [SMILES: COC(=O)c1nc(Cl)ccc1N[C@H](C)c1cc(C)cc2c(=O)c(C)c(-c3cccnc3)oc12 ; Cc1cc([C@@H](C)Nc2cccnc2C(F)(F)F)c2oc(-c3ccccc3)c(C)c(=O)c2c1 ; Cc1cc([C@@H](C)Nc2ccccc2[N+](=O)[O-])c2oc(-c3ccccc3)c(C)c(=O)c2c1 ; Cc1cc([C@@H](C)Nc2ccccc2NS(C)(=O)=O)c2oc(-c3ccccc3)c(C)c(=O)c2c1 ; Cc1cc([C@@H](C)Nc2cccnc2N2CCCC2=O)c2oc(-c3ccccc3)c(C)c(=O)c2c1]

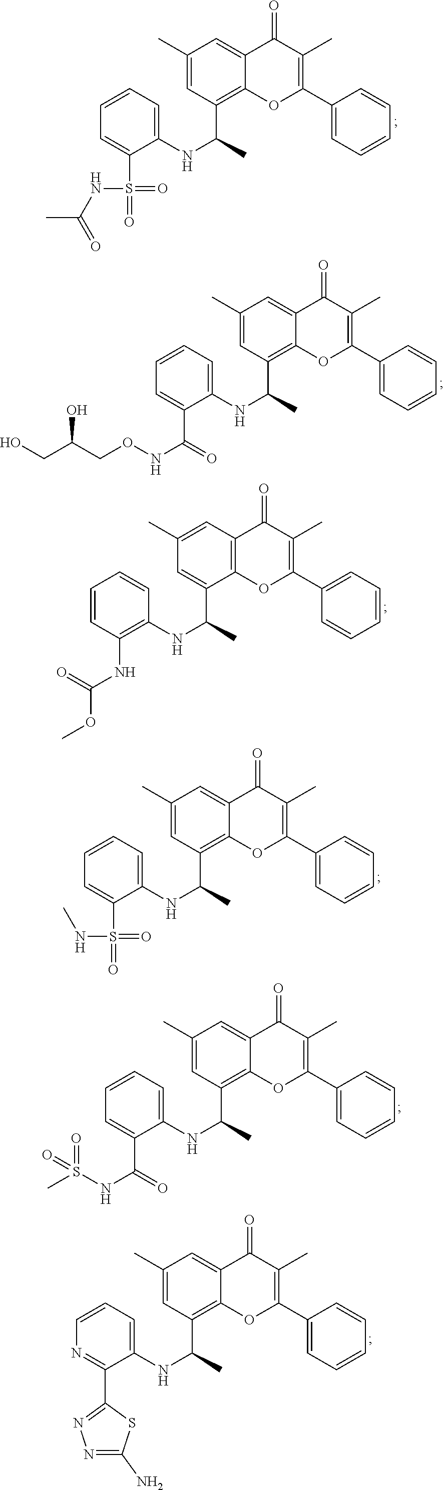 [SMILES: CC(=O)NS(=O)(=O)c1ccccc1N[C@H](C)c1cc(C)cc2c(=O)c(C)c(-c3ccccc3)oc12 ; Cc1cc([C@@H](C)Nc2ccccc2C(=O)NOC[C@H](O)CO)c2oc(-c3ccccc3)c(C)c(=O)c2c1 ; COC(=O)Nc1ccccc1N[C@H](C)c1cc(C)cc2c(=O)c(C)c(-c3ccccc3)oc12 ; CNS(=O)(=O)c1ccccc1N[C@H](C)c1cc(C)cc2c(=O)c(C)c(-c3ccccc3)oc12 ; Cc1cc([C@@H](C)Nc2ccccc2C(=O)NS(C)(=O)=O)c2oc(-c3ccccc3)c(C)c(=O)c2c1 ; Cc1cc([C@@H](C)Nc2cccnc2-c2nnc(N)s2)c2oc(-c3ccccc3)c(C)c(=O)c2c1]

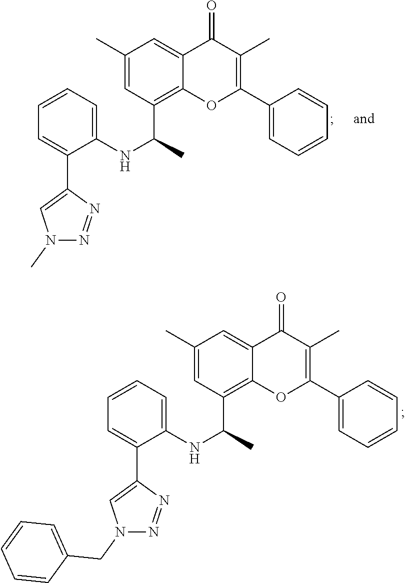 [SMILES: Cc1cc([C@@H](C)Nc2ccccc2-c2cn(C)nn2)c2oc(-c3ccccc3)c(C)c(=O)c2c1 ; Cc1cc([C@@H](C)Nc2ccccc2-c2cn(Cc3ccccc3)nn2)c2oc(-c3ccccc3)c(C)c(=O)c2c1]

or a pharmaceutically acceptable salt thereof.

In yet a further compound of Formula (I), the compound is selected from:

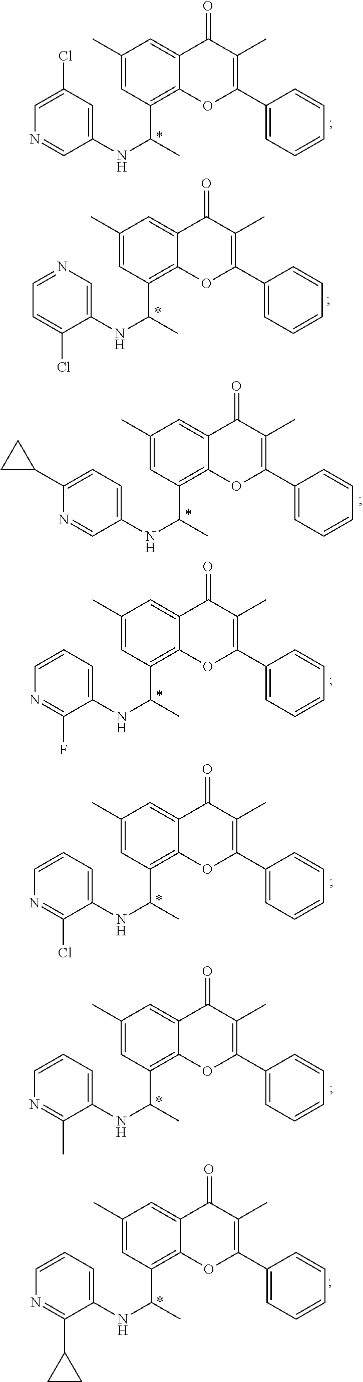

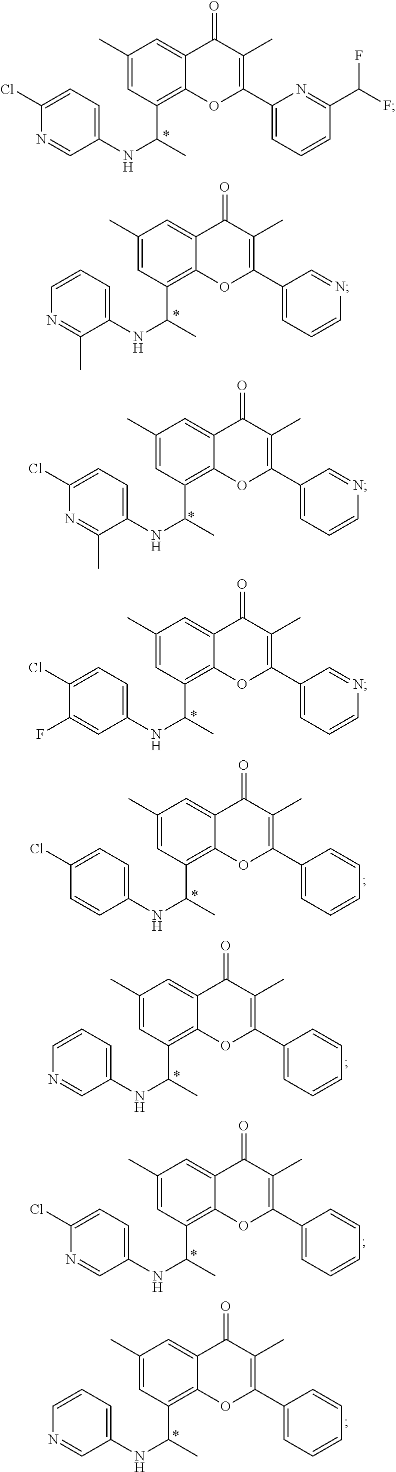

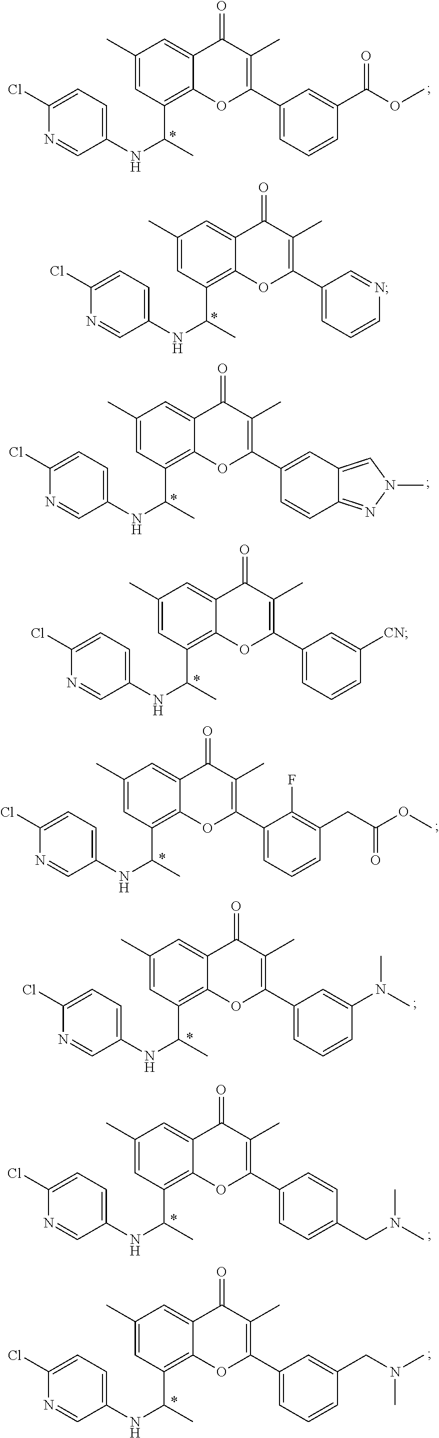

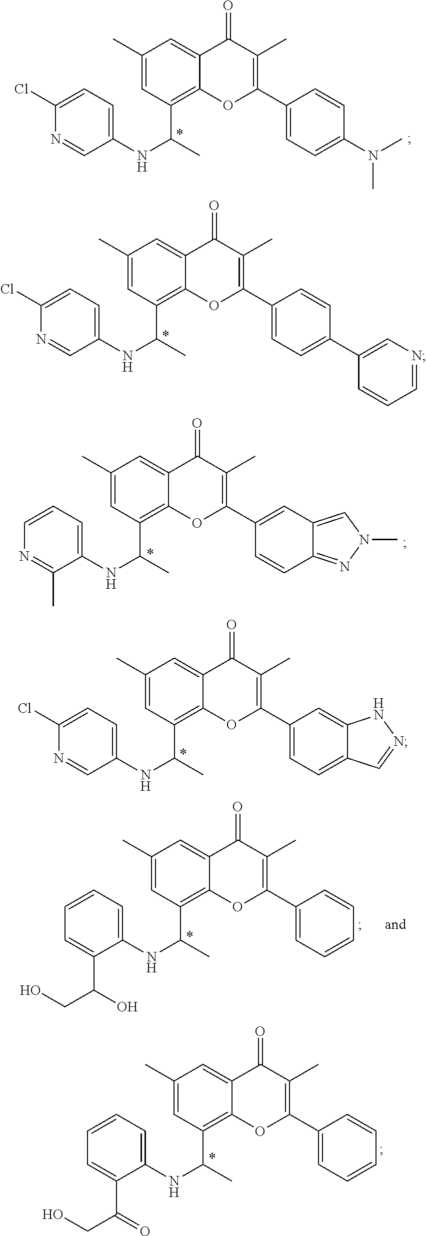 [SMILES: Cc1cc(C(C)Nc2ccc(Cl)nc2)c2oc(-c3ccc(N(C)C)cc3)c(C)c(=O)c2c1 ; Cc1cc(C(C)Nc2ccc(Cl)nc2)c2oc(-c3ccc(-c4cccnc4)cc3)c(C)c(=O)c2c1 ; Cc1cc(C(C)Nc2cccnc2C)c2oc(-c3ccc4nn(C)cc4c3)c(C)c(=O)c2c1 ; Cc1cc(C(C)Nc2ccc(Cl)nc2)c2oc(-c3ccc4cn[nH]c4c3)c(C)c(=O)c2c1 ; Cc1cc(C(C)Nc2ccccc2C(O)CO)c2oc(-c3ccccc3)c(C)c(=O)c2c1 ; Cc1cc(C(C)Nc2ccccc2C(=O)CO)c2oc(-c3ccccc3)c(C)c(=O)c2c1]

or a pharmaceutically acceptable salt of any of the foregoing; wherein the bond at the * position is as represented,

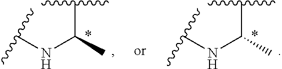

In yet a further compound of Formula (I), the compound is selected from

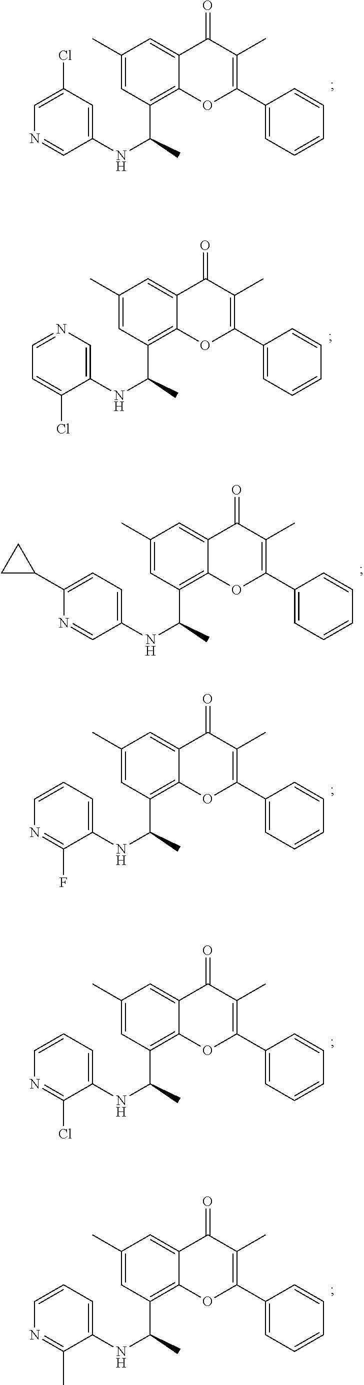 [SMILES: Cc1cc([C@@H](C)Nc2cncc(Cl)c2)c2oc(-c3ccccc3)c(C)c(=O)c2c1 ; Cc1cc([C@@H](C)Nc2cnccc2Cl)c2oc(-c3ccccc3)c(C)c(=O)c2c1 ; Cc1cc([C@@H](C)Nc2ccc(C3CC3)nc2)c2oc(-c3ccccc3)c(C)c(=O)c2c1 ; Cc1cc([C@@H](C)Nc2cccnc2F)c2oc(-c3ccccc3)c(C)c(=O)c2c1 ; Cc1cc([C@@H](C)Nc2cccnc2Cl)c2oc(-c3ccccc3)c(C)c(=O)c2c1 ; Cc1cc([C@@H](C)Nc2cccnc2C)c2oc(-c3ccccc3)c(C)c(=O)c2c1]

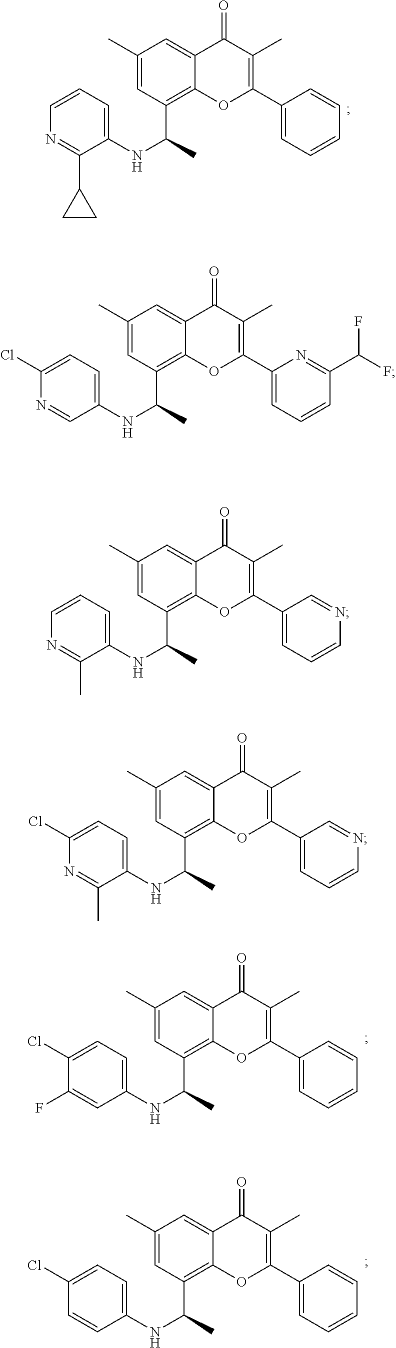 [SMILES: Cc1cc([C@@H](C)Nc2cccnc2C2CC2)c2oc(-c3ccccc3)c(C)c(=O)c2c1 ; Cc1cc([C@@H](C)Nc2ccc(Cl)nc2)c2oc(-c3cccc(C(F)F)n3)c(C)c(=O)c2c1 ; Cc1cc([C@@H](C)Nc2cccnc2C)c2oc(-c3cccnc3)c(C)c(=O)c2c1 ; Cc1cc([C@@H](C)Nc2ccc(Cl)nc2C)c2oc(-c3cccnc3)c(C)c(=O)c2c1 ; Cc1cc([C@@H](C)Nc2ccc(Cl)c(F)c2)c2oc(-c3ccccc3)c(C)c(=O)c2c1 ; Cc1cc([C@@H](C)Nc2ccc(Cl)cc2)c2oc(-c3ccccc3)c(C)c(=O)c2c1]

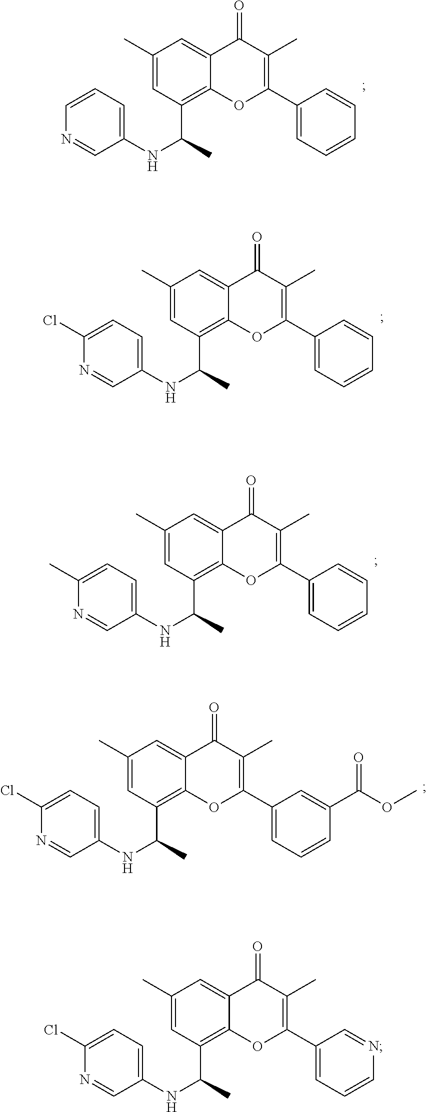 [SMILES: Cc1cc([C@@H](C)Nc2cccnc2)c2oc(-c3ccccc3)c(C)c(=O)c2c1 ; Cc1cc([C@@H](C)Nc2ccc(Cl)nc2)c2oc(-c3ccccc3)c(C)c(=O)c2c1 ; Cc1cc([C@@H](C)Nc2ccc(C)nc2)c2oc(-c3ccccc3)c(C)c(=O)c2c1 ; COC(=O)c1cccc(-c2oc3c([C@@H](C)Nc4ccc(Cl)nc4)cc(C)cc3c(=O)c2C)c1 ; Cc1cc([C@@H](C)Nc2ccc(Cl)nc2)c2oc(-c3cccnc3)c(C)c(=O)c2c1]

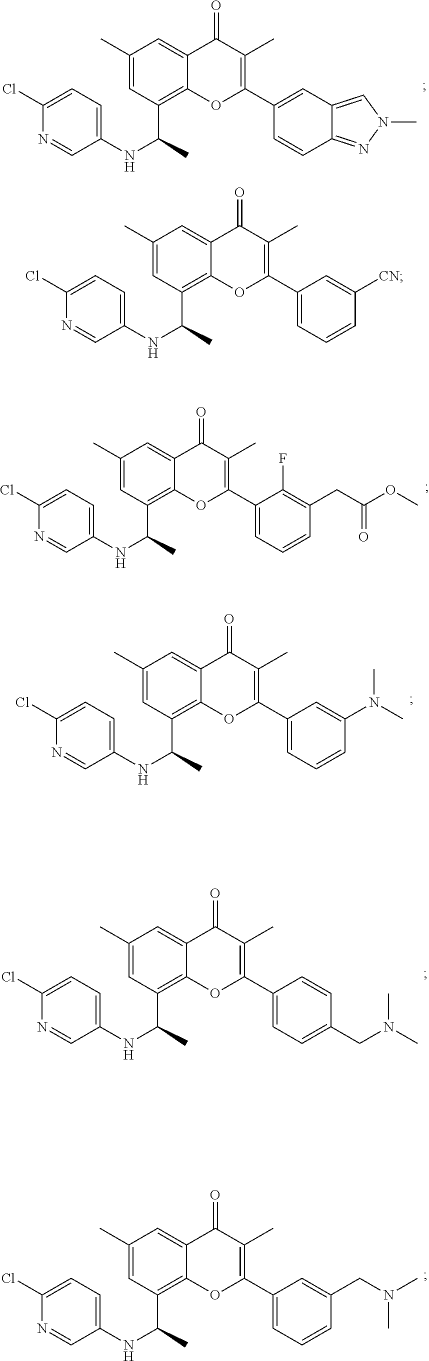 [SMILES: Cc1cc([C@@H](C)Nc2ccc(Cl)nc2)c2oc(-c3ccc4nn(C)cc4c3)c(C)c(=O)c2c1 ; Cc1cc([C@@H](C)Nc2ccc(Cl)nc2)c2oc(-c3cccc(C#N)c3)c(C)c(=O)c2c1 ; COC(=O)Cc1cccc(-c2oc3c([C@@H](C)Nc4ccc(Cl)nc4)cc(C)cc3c(=O)c2C)c1F ; Cc1cc([C@@H](C)Nc2ccc(Cl)nc2)c2oc(-c3cccc(N(C)C)c3)c(C)c(=O)c2c1 ; Cc1cc([C@@H](C)Nc2ccc(Cl)nc2)c2oc(-c3ccc(CN(C)C)cc3)c(C)c(=O)c2c1 ; Cc1cc([C@@H](C)Nc2ccc(Cl)nc2)c2oc(-c3cccc(CN(C)C)c3)c(C)c(=O)c2c1]

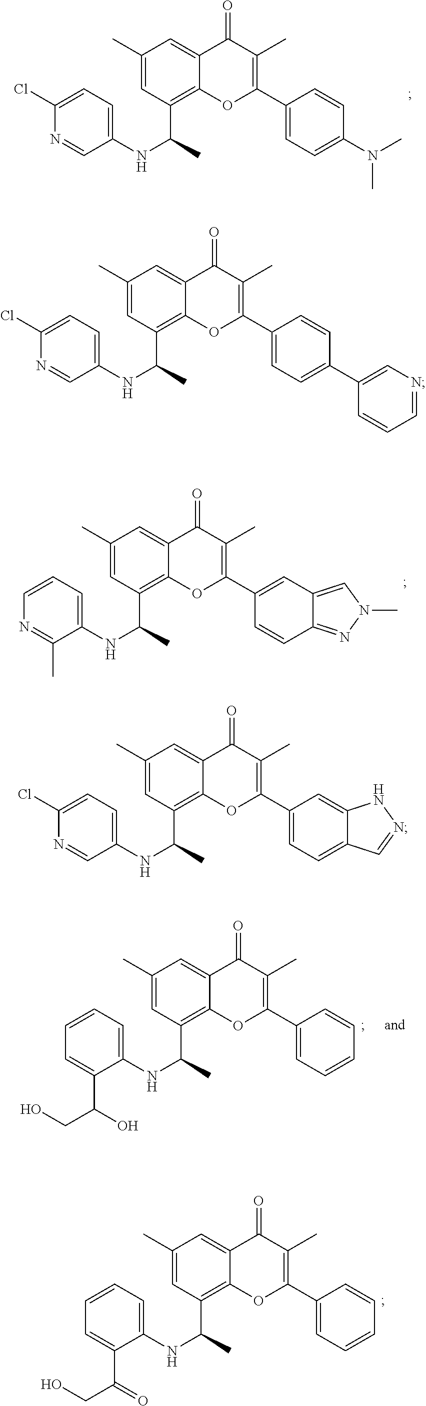 [SMILES: Cc1cc([C@@H](C)Nc2ccc(Cl)nc2)c2oc(-c3ccc(N(C)C)cc3)c(C)c(=O)c2c1 ; Cc1cc([C@@H](C)Nc2ccc(Cl)nc2)c2oc(-c3ccc(-c4cccnc4)cc3)c(C)c(=O)c2c1 ; Cc1cc([C@@H](C)Nc2cccnc2C)c2oc(-c3ccc4nn(C)cc4c3)c(C)c(=O)c2c1 ; Cc1cc([C@@H](C)Nc2ccc(Cl)nc2)c2oc(-c3ccc4cn[nH]c4c3)c(C)c(=O)c2c1 ; Cc1cc([C@@H](C)Nc2ccccc2C(O)CO)c2oc(-c3ccccc3)c(C)c(=O)c2c1 ; Cc1cc([C@@H](C)Nc2ccccc2C(=O)CO)c2oc(-c3ccccc3)c(C)c(=O)c2c1]

or a pharmaceutically acceptable salt thereof.

In yet a further compound of Formula (I), the compound is selected from

[chemical structure]

[chemical structure]

[chemical structure]

[chemical structure]

[chemical structure]

[chemical structure]

[chemical structure]

[chemical structure]

[chemical structure]

[chemical structure]

[chemical structure]

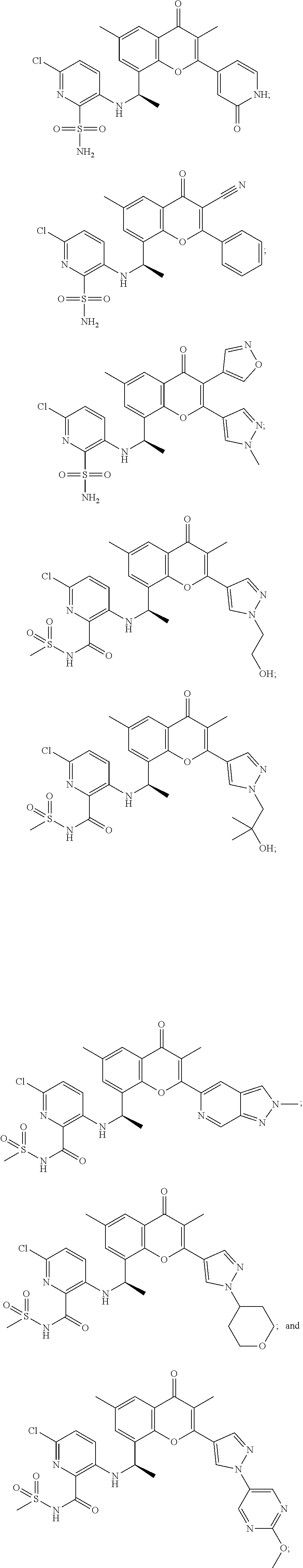

or a pharmaceutically acceptable salt thereof.

In a further embodiment of the compound of Formula (I) or a pharmaceutically acceptable salt thereof, the compound is an isotopic derivative of any one of the compounds described herein or a pharmaceutically acceptable salt thereof. It is understood that the isotopic derivative can be prepared using any of a variety of art-recognized techniques. For example, the isotopic derivatives can generally be prepared by carrying out the procedures disclosed in the schemes and/or in the examples described herein or a pharmaceutically acceptable salt thereof, by substituting an isotopically labeled reagent for a non-isotopically labeled reagent. In the compounds of this invention any atom not specifically designated as a particular isotope is meant to represent a stable isotope of that atom.

In a further embodiment of a compound of Formula (I) or a pharmaceutically acceptable salt thereof, the compound is deuterated at one or more positions. Unless otherwise stated, when an atom is designated specifically as “H” or “hydrogen”, the atom is understood to have hydrogen at its natural abundance isotopic composition. Also, unless otherwise stated, when an atom is designated specifically as “D” or “deuterium”, the atom is understood to have deuterium at an abundance substantially greater than the natural abundance of deuterium, which is 0.015%.

In an embodiment of a compound of Formula (I) or pharmaceutically acceptable salt thereof, R₈ is —H, -D, or C₁-C₆ alkyl. In an embodiment of a compound of Formula (I) or pharmaceutically acceptable salt thereof, the compound is selected from:

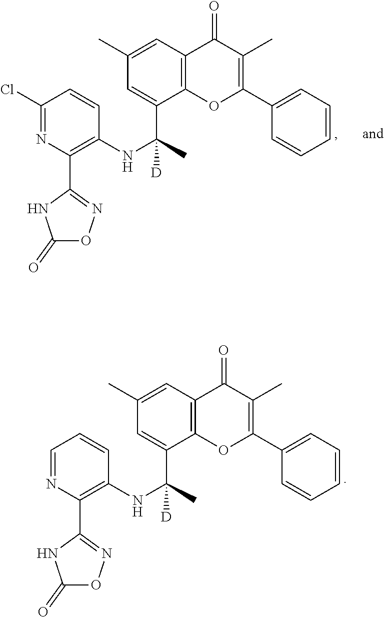 [SMILES: [2H][C@](C)(Nc1ccc(Cl)nc1-c1noc(=O)[nH]1)c1cc(C)cc2c(=O)c(C)c(-c3ccccc3)oc12 ; [2H][C@](C)(Nc1cccnc1-c1noc(=O)[nH]1)c1cc(C)cc2c(=O)c(C)c(-c3ccccc3)oc12]

In an embodiment of a compound of Formula (I) or a pharmaceutically acceptable salt thereof, R₂ is 1-(trideuteriomethyl)pyrazol-4-yl. In an embodiment of a compound of Formula (I) or pharmaceutically acceptable salt thereof, the compound is:

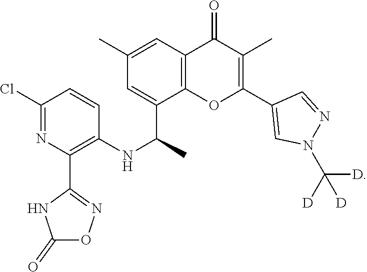

A pharmaceutically acceptable salt of a compound of the present invention is, for example, an acid-addition salt of a compound of the invention, which is sufficiently basic, for example, an acid-addition salt with, for example, an inorganic or organic acid, for example hydrochloric, hydrobromic, sulfuric, phosphoric, trifluoroacetic, formic, citric, methane sulfonate or maleic acid. In addition, a pharmaceutically acceptable salt of a compound of the present invention which is sufficiently acidic is an alkali metal salt, for example a sodium or potassium salt, an alkaline earth metal salt, for example a calcium or magnesium salt, an ammonium salt or a salt with an organic base which affords a pharmaceutically acceptable cation, for example a salt with methylamine, dimethylamine, diethylamine, trimethylamine, piperidine, morpholine or tris-(2-hydroxyethyl)amine. Pharmaceutically acceptable salts, and common methodology for preparing them are well known in the art (see, e.g., P. Stahl, et al. Handbook of Pharmaceutical Salts: Properties, Selection, and Use, 2ⁿd Revised Edition (Wiley-VCH, 2011); S. M. Berge, et al., “Pharmaceutical Salts,” Journal of Pharmaceutical Sciences, Vol. 66, No. 1, January 1977).

Further representative “pharmaceutically acceptable salts” include, e.g., water-soluble, and water-insoluble salts, such as the acetate, amsonate (4,4-diaminostilbene-2,2-disulfonate), benzenesulfonate, benzoate, bicarbonate, bisulfate, bitartrate, borate, bromide, butyrate, calcium, calcium edetate, camsylate, carbonate, chloride, citrate, clavulanate, dihydrochloride, edetate, edisylate, estolate, esylate, fumarate, gluceptate, gluconate, glutamate, glycollylarsanilate, hexafluorophosphate, hexylresorcinate, hydrabamine, hydrobromide, hydrochloride, hydroxynaphthoate, iodide, isothionate, lactate, lactobionate, laurate, magnesium, malate, maleate, mandelate, mesylate, methylbromide, methylnitrate, methylsulfate, mucate, napsylate, nitrate, N-methylglucamine ammonium salt, 3-hydroxy-2-naphthoate, oleate, oxalate, palmitate, pamoate, pantothenate, phosphate/diphosphate, picrate, polygalacturonate, propionate, p-toluenesulfonate, salicylate, stearate, subacetate, succinate, sulfate, sulfosalicylate, tannate, tartrate, teoclate, tosylate, triethiodide, and valerate salts.

The compounds of the present invention can be prepared in a number of ways well known to those skilled in the art of organic synthesis. By way of example, compounds of the present invention can be synthesized using the methods described below, together with synthetic methods known in the art of synthetic organic chemistry, or variations thereon as appreciated by those skilled in the art. Preferred methods include but are not limited to those methods described below.

### Further Aspects

The following are further numbered aspects of the invention:

1. A compound of a Formula (I):

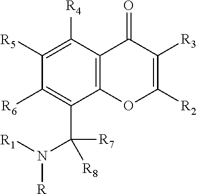

or a pharmaceutically acceptable salt thereof, wherein:
R and R₁ together with the nitrogen to which they are attached form an optionally substituted bicyclic ring selected from dihydrobenzimidazolone, dihydroindazolone, indolinone or quinazolinone; wherein the optionally substituted bicyclic ring is optionally substituted with one to three substituents each independently selected from oxo, —CN, halogen, C₁-C₆ alkyl, C₁-C₆ haloalkyl, —OH or C₁-C₆ alkoxy; or
R is —H or C₁-C₃ alkyl;
R₁ is an optionally substituted bicyclic ring selected from isobenzofuranone, benzofuranone, indole, isoindolinone, indolinone, quinazolinone, 3,4-dihydro-2H-isoquinolin-1-one, 2H-isoquinolin-1-one, imidaza[1,2-A]pyridine, or benzothiazolone; wherein the optionally substituted bicyclic ring is optionally substituted with one to three substituents each independently selected from oxo, —CN, halogen, C₁-C₆ alkyl, C₁-C₆ haloalkyl, —OH or C₁-C₆ alkoxy; or
R₁ is a group of the formula:

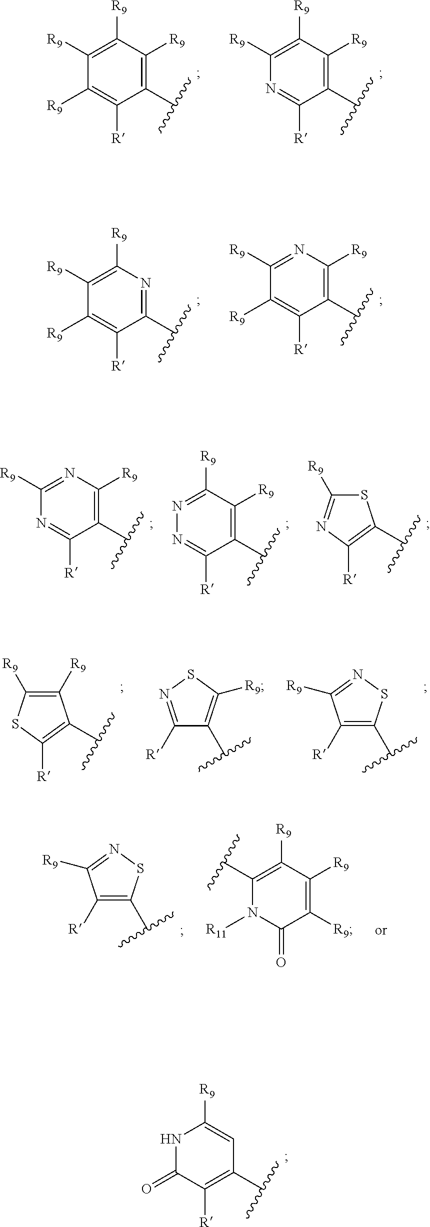

R′ is hydrogen, halogen, C₁-C₃ alkyl, C₁-C₃ haloalkyl, C₁-C₃ hydroxyalkyl, C₁-C₃ alkoxy, C₁-C₃ haloalkoxy, —OH, —CH(OH)—CH₂OH, —CH(OH)C₁-C₃ haloalkyl, —CO—CH₂OH, C₃-C₆ cycloalkyl, —NO₂, —NR₁₁R₁₁, —N(R₁₁)—CO₂C₁-C₃ alkyl, —N(R)—SO₂C₁-C₃ alkyl, —N(R)—SO₂R₁₅, —SO₂C₁-C₃ alkyl, —SOC₁-C₃ alkyl, —SO₂NR₁₁R₁₁, —SO₂N(R₁₁)—CO—C₁-C₃ alkyl, —SO₂N(R₁₁)—CN, —SO₂N(R₁₁)(R₁₃)—C(═N—OH)—NH₂, —CN, —CO₂C₁-C₃ alkyl, —CONR₁₁R₁₂, —CON(R)—(CH₂)ₙ—R₁₃, —CO—SR₁₂, —CO—NHSO₂R₁₆, —COCH═SOR₁₁(R₁₁), or —COCH₂CN, or a group of the formula:

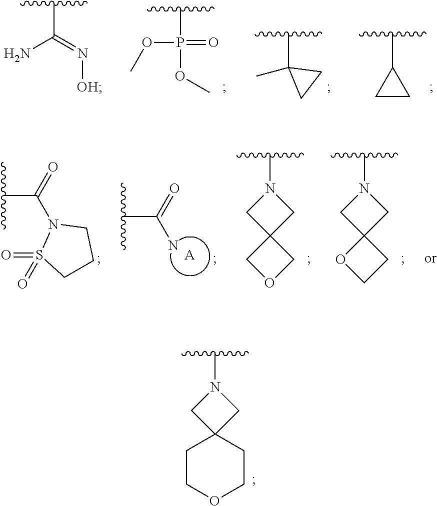

wherein ring A is pyrrolidine optionally substituted with a —CN; or
R′ is selected from oxetane, azetidine, pyrrolidine, tetrahydrofuran, morpholine, thiomorpholine, piperidine, piperazine, pyrrole, furan, thiophene, pyrazole, imidazole, isoxazole, oxazole, isothiazole, thiazole, triazole, oxadiazole, 1,2,4-oxadiazolin-5-one, thiadiazole, tetrazole, phenyl, pyridine, pyridazine, pyrimidine, pyrazine, or triazine; each of which is optionally substituted with one to three substituents independently selected from oxo, —OH, —NR₁₁R₁₁, —N(R₁₁)—C(O)—R₁₁, —N(R₁₁)—CN, —OR₁₁, —CN, halogen, morpholine, oxetane, C₁-C₆ haloalkyl or C₁-C₆ alkyl optionally substituted with an aryl, a 5-member heteroaryl or a 6-member heteroaryl; or
R′ is a group of the formula:

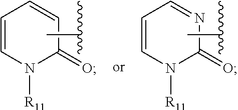

R₂ is a group of the formula:

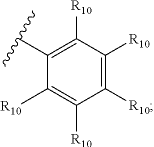

or
R₂ is a group of the formula:

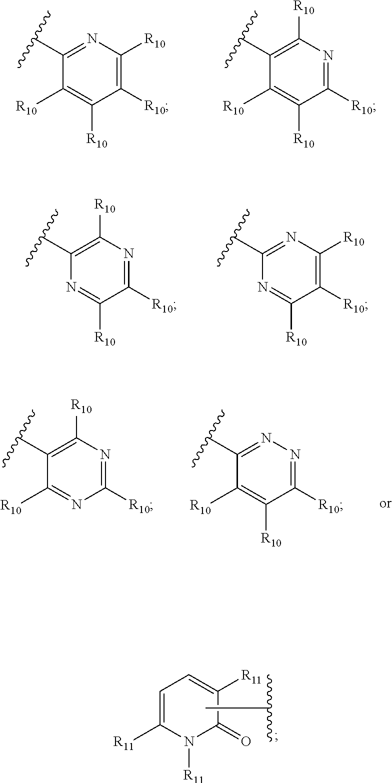

or
R₂ is an optionally substituted 5-member ring heteroaryl selected from pyrrole, furan, thiophene, pyrazole, isoxazole, isothiazole, imidazole, oxazole, thiazole, triazole, tetrazole, oxadiazole, and thiadiazole; wherein the optionally substituted 5-member ring heteroaryl is optionally substituted with one to three substituents each independently selected from —CN, halogen, C₁-C₆ haloalkyl, C₁-C₆ alkoxy, C₁-C₆ haloalkoxy, —SO₂R₁₁, —CO₂C₁-C₃ alkyl, —C(O)NR₁₁R₁₁, —OH, —NR₁₁R₁₁, —NR₁₁CO₂R₁₁, —CD₃, an optionally substituted C₁-C₆ alkyl, an optionally substituted C₂-C₆ alkenyl, an optionally substituted C₂-C₆ alkynyl, an optionally substituted C₃-C₅ cycloalkyl, an optionally substituted heterocycle selected from pyrrolidine, pyrrolidinone, piperidine, tetrahydropyran, oxetane, azetidine, or morpholine, an optionally substituted phenyl, an optionally substituted 1,3-benzodioxole, an optionally substituted 2,3-dihydro-1,4-benzodioxine, or an optionally substituted heteroaryl selected from pyridine, pyrimidine, pyridazine, pyrazine, pyrazole, isoxazole, isothiazole, imidazole, oxazole, or thiazole; wherein the optionally substituted C₁-C₆ alkyl, C₂-C₆ alkenyl, or C₂-C₆ alkynyl is each optionally substituted with a —CN, —OH, oxetanyl, C₁-C₃ alkoxy, —CH₂CH(OH)CH(OH)R₁₁, or —CONR₁₁R₁₁; the optionally substituted C₃-C₅ cycloalkyl, phenyl, 1,3-benzodioxole, 2,3-dihydro-1,4-benzodioxine, heterocycle or heteroaryl is each optionally substituted with one to three substituents each independently selected from halogen, C₁-C₃ alkyl, C₁-C₃ haloalkyl, C₁-C₃ alkoxy, C₁-C₃ haloalkoxy, —C(O)(CH₂)ₙOR₁₁, C(O)C(CH₃)₂OH, —SO₂R₁₁, —NR₁₁R₁₁, —OH or —CN; or R₂ is an optionally substituted bicyclic ring selected from 1,3-benzodioxole, 2,3-dihydro-1,4-benzodioxine, indole, indazole, isoindazole, isoindolin-1-one, indolin-2-one, benzoxazole, benzotriazole, benzo[d]oxazol-2(3H)-one, 1,3-dihydro-2H-pyrrolo[2,3-b]138yridine-2-one, 6,7-dihydro-4H-pyrazolo[5,1-c][1,4]oxazine, 4,5,6,7-tetrahydropyrazolo[1,5-a]pyrazine, or 2,3-dihydro-[1,4]dioxino[2,3-b]pyridine, imidazo[1,2-a]pyridine, pyrazolo[4,3-b]pyridine, pyrazolo[3,4-b]pyridine, pyrazolo[3,4-c]pyridine, pyrazolo[1,5-a]pyrimidine, or an optionally substituted bicyclic heteroaryl of 8 to 10 ring atoms containing 1, 2, 3, 4, or 5 ring heteroatoms independently selected from N, O, or S; wherein the optionally substituted bicyclic ring is optionally substituted with one to three substituents each independently selected from halogen and C₁-C₆ alkyl; the optionally substituted bicyclic heteroaryl is optionally substituted with one to three substituents each independently selected from —CN, halogen, C₁-C₆ haloalkyl, C₁-C₆ alkoxy, C₁-C₆ haloalkoxy, —SO₂R₁₁, —CO₂CH, —CO₂C₁-C₃ alkyl, —CONR₁₁R₁₁, —NR₁₁R₁₁, —NR₁₁CO₂R₁₁, —OH, an optionally substituted C₁-C₆ alkyl, an optionally substituted C₂-C₆ alkenyl, an optionally substituted C₂-C₆ alkynyl, an optionally substituted C₃-C₅ cycloalkyl, an optionally substituted heterocycle selected from pyrrolidine, pyrrolidinone, piperidine or morpholine, an optionally substituted phenyl, an optionally substituted 1,3-benzodioxole, an optionally substituted 2,3-dihydro-1,4-benzodioxine, or an optionally substituted heteroaryl selected from pyridine, pyrimidine, pyridazine, pyrazine, pyrazole, isoxazole, isothiazole, imidazole, oxazole, or thiazole; wherein the optionally substituted C₁-C₆ alkyl, C₂-C₆ alkenyl, or C₂-C₆ alkynyl is each optionally substituted with a —CN, —OH, oxetanyl, C₁-C₃ alkoxy, —C(O)NR₁₁R₁₁ or phenyl; the optionally substituted C₃-C₅ cycloalkyl, phenyl, 1,3-benzodioxole, 2,3-dihydro-1,4-benzodioxine, heterocycle or heteroaryl is each optionally substituted with one to three substituents each independently selected from halogen, C₁-C₃ alkyl, C₁-C₃ haloalkyl, C₁-C₃ alkoxy, C₁-C₃ haloalkoxy, —SO₂R₁₁, —NR₁₁R₁₁, —OH or —CN; or
R₂ is a cyclopropyl, cyclobutyl, cyclopentyl, bicyclo[1.1.1]pentane, bicyclo[2.2.2]octane, or cyclohexyl, each optionally substituted with one to three R₁₀ substituents, or R₂ is a group of the formula:

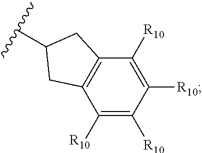

or
R₃ is —H, halogen, —CN, —C(CN)═CHOH, —N(H)(C₁-C₃ alkyl), —N(C₁-C₃ alkyl)₂, —N(H)(CH₂CH₂CO₂H), —CO—C₁-C₃ alkyl, C₁-C₆ alkyl, C₁-C₆ haloalkyl, C₁-C₆ hydroxyalkyl, C₃-C₅ cycloalkyl, an optionally substituted heterocycle of 3 to 5 ring atoms containing 1, 2, or 3 ring heteroatoms independently selected from N, O, or S, or an optionally substituted heteroaryl of 5 or 6 ring atoms containing 1, 2, or 3 ring heteroatoms independently selected from N, O, or S; wherein the optionally substituted heterocycle or heteroaryl is each optionally substituted with one to three substituents each independently selected from halogen, C₁-C₃ alkyl, or C₁-C₃ haloalkyl; each of R₄, R₅ and R₆ is independently —H, halogen, —CN, C₁-C₆ alkyl or C₁-C₆ haloalkyl;
R₇ is —CN, C₁-C₆ alkyl, —CH₂OH or C₁-C₆ haloalkyl;
R₈ is —H, -D, or C₁-C₆ alkyl;
each R₉ is independently —H, halogen, —CN, C₁-C₆ alkyl, C₁-C₆ haloalkyl, C₁-C₆ alkoxy, or C₃-C₅ cycloalkyl;
each R₁₀ is independently —H, —CN, halogen, C₁-C₆ haloalkyl, C₁-C₆ alkoxy, C₁-C₆ haloalkoxy, —SO₂R₁₁, —SONR₁₁R₁₁, —CO₂H, —CO₂C₁-C₃ alkyl, —C(O)NR₁₁R₁₂, —NR₁₁R₁₁, —NR₁₁—CO₂R₁₁, —N(R₁₁)COR₁₁, —OH, an optionally substituted C₁-C₆ alkyl, an optionally substituted C₂-C₆ alkenyl, an optionally substituted C₂-C₆ alkynyl, an optionally substituted C₃-C₅ cycloalkyl, an optionally substituted heterocycle selected from pyrrolidine, pyrrolidinone, piperidine or morpholine, an optionally substituted phenyl, an optionally substituted 1,3-benzodioxole, an optionally substituted 2,3-dihydro-1,4-benzodioxine, or an optionally substituted heteroaryl selected from pyrazole, isoxazole, isothiazole, imidazole, oxazole, thiazole, or pyridine, or a group of the formula:

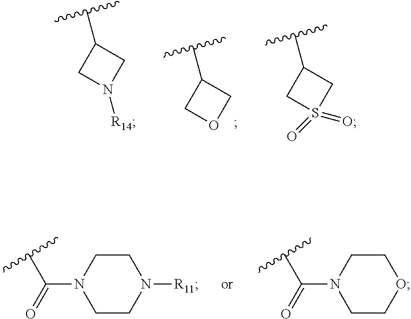

wherein the optionally substituted C₁-C₆ alkyl, C₂-C₆ alkenyl, or C₂-C₆ alkynyl is each optionally substituted with a —CN, —OH, oxetanyl, C₁-C₃ alkoxy, or —CONR₁₁R₁₁; the optionally substituted C₃-C₅ cycloalkyl, phenyl, 1,3-benzodioxole, 2,3-dihydro-1,4-benzodioxine, heterocycle or heteroaryl is each optionally substituted with one to three substituents each independently selected from halogen, C₁-C₃ alkyl, C₁-C₃ haloalkyl, C₁-C₃ alkoxy, C₁-C₃ haloalkoxy, —SO₂R₁₁, —NR₁₁R₁₁, —OH or —CN;
each R₁₁ is independently —H, C₁-C₃ alkyl, C₃-C₇ cycloalkyl, or C₁-C₃ haloalkyl;
each R₁₂ is independently —H, optionally substituted C₁-C₃ alkyl, C₃-C₆ alkoxy, C₃-C₆ cycloalkyl, —SO₂C₁-C₃ alkyl, —SO₂C₁-C₃ haloalkyl, —SO₂NR₁₁R₁₁, —NR₁₁R₁₁, —OR₁₁, —O—CH₂—CH(OH)—CH₂OH, —CN, oxetane, tetrahydrofuran, an aryl, a 5-member heteroaryl optionally substituted with a methyl, a 6-member heteroaryl, or a group of the formula

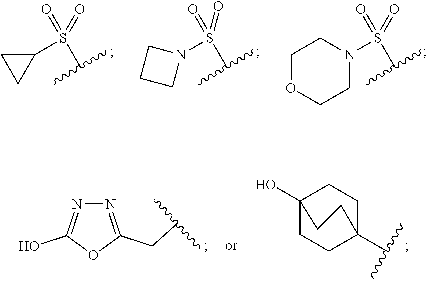 [SMILES: *S(=O)(=O)C1CC1 ; *S(=O)(=O)N1CCC1 ; *S(=O)(=O)N1CCOCC1 ; *Cc1nnc(O)o1 ; *C12CCC(O)(CC1)CC2]

wherein the optionally substituted C₁-C₃ alkyl is optionally substituted with —OH, a C₃-C₆ cycloalkyl, oxetane, tetrahydrofuran, an aryl, a 5-member heteroaryl, a 6-member heteroaryl or an indole;
R₁₃ is —NR₁₁R₁₁, —OR₁₁, —SO₂C₁-C₃ alkyl, or a ring selected from oxetane, tetrahydrofuran or oxadiazole wherein the ring is optionally substituted with —NR₁₁R₁₁, or —OR₁₁;
R₁₄ is —H, optionally substituted C₁-C₃ alkyl, —SO₂C₁-C₃ alkyl, an aryl, a 5-member heteroaryl, or a 6-member heteroaryl; wherein the optionally substituted C₁-C₃ alkyl is optionally substituted with an aryl, a 5-member heteroaryl or a 6-member heteroaryl;
R₁₅ is an optionally substituted aryl, or an optionally substituted 6-member heteroaryl; wherein the optionally substituted aryl, or the optionally substituted 6-member heteroaryl is each optionally substituted with one to three substituents each independently selected from halogen, C₁-C₃ alkyl, or C₁-C₃ haloalkyl;
R₁₆ is H, C₁-C₃ alkyl, —NH₂, phenyl, or pyridine, and
n is 0, 1 or 2.
   2. The compound of claim 1, or pharmaceutically acceptable salt thereof, wherein:
R and R₁ together with the nitrogen to which they are attached form an optionally substituted bicyclic ring selected from dihydrobenzimidazolone, dihydroindazolone, indolinone or quinazolinone; wherein the optionally substituted bicyclic ring is optionally substituted with one to three substituents each independently selected from oxo, —CN, halogen, C₁-C₆ alkyl, C₁-C₆ haloalkyl, —OH or C₁-C₆ alkoxy; or
R is —H or C₁-C₃ alkyl;
R₁ is an optionally substituted bicyclic ring selected from isobenzofuranone, benzofuranone, isoindolinone, indolinone, quinazolinone, 3,4-dihydro-2H-isoquinolin-1-one, 2H-isoquinolin-1-one, or benzothiazolone; wherein the optionally substituted bicyclic ring is optionally substituted with one to three substituents each independently selected from oxo, —CN, halogen, C₁-C₆ alkyl, C₁-C₆ haloalkyl, —OH or C₁-C₆ alkoxy; or
R₁ is a group of the formula:

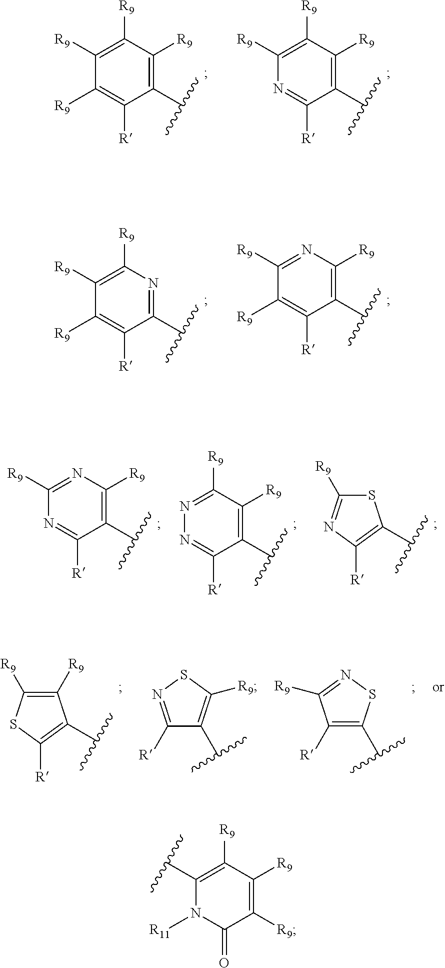

R′ is hydrogen, halogen, C₁-C₃ alkyl, C₁-C₃ haloalkyl, C₁-C₃ alkoxy, C₁-C₃ haloalkoxy, —OH, —CH(OH)—CH₂OH, —CO—CH₂OH, C₃-C₆ cycloalkyl, —NO₂, —N(R₁₁)—CO₂C₁-C₃ alkyl, —N(R₁₁)—SO₂C₁-C₃ alkyl, —N(R₁₁)—SO₂R₁₅, —SO₂C₁-C₃ alkyl, —SO₂NR₁₁R₁₁, —SO₂N(R₁₁)—CO—C₁-C₃ alkyl, —SO₂N(R₁₁)—CN, —C(═N—OH)—NH₂, —CN, —CO₂C₁-C₃ alkyl, —CONR₁₁R₁₂, —CON(R₁₁)—(CH₂)ₙ—R₁₃, —CO—SR₁₂, or a group of the formula:

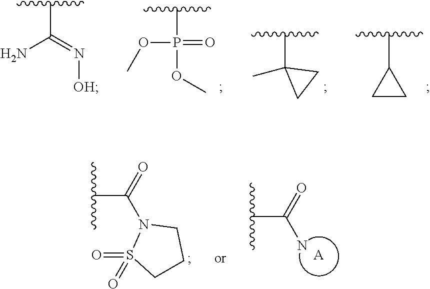

wherein ring A is pyrrolidine optionally substituted with a —CN; or
R′ is selected from oxetane, pyrrolidine, tetrahydrofuran, morpholine, piperidine, piperazine, pyrrole, furan, thiophene, pyrazole, imidazole, isoxazole, oxazole, isothiazole, thiazole, triazole, oxadiazole, thiadiazole, tetrazole, phenyl, pyridine, pyridazine, pyrimidine, pyrazine, or triazine; each of which is optionally substituted with one to three substituents independently selected from oxo, —OH, —NR₁₁R₁₁, —N(R₁₁)—CO—R₁₁, —N(R₁₁)—CN, —OR₁₁, —CN, halogen, C₁-C₆ haloalkyl or C₁-C₆ alkyl optionally substituted with an aryl, a 5-member heteroaryl or a 6-member heteroaryl; or
R′ is a group of the formula:

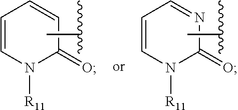

R₂ is a group of the formula:

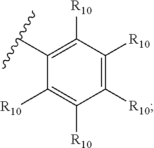

or
R₂ is a group of the formula:

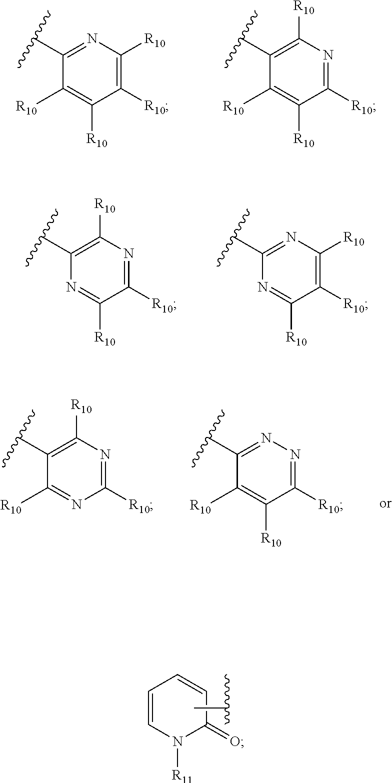

or
R₂ is an optionally substituted 5-member ring heteroaryl selected from pyrrole, furan, thiophene, pyrazole, isoxazole, isothiazole, imidazole, oxazole, thiazole, triazole, tetrazole, oxadiazole, and thiadiazole; wherein the optionally substituted 5-member ring heteroaryl is optionally substituted with one to three substituents each independently selected from —CN, halogen, C₁-C₆ haloalkyl, C₁-C₆ alkoxy, C₁-C₆ haloalkoxy, —SO₂R₁₁, —CO₂C₁-C₃ alkyl, —CONR₁₁R₁₁, —OH, —NR₁₁R₁₁, —NR₁₁CO₂R₁₁, an optionally substituted C₁-C₆ alkyl, an optionally substituted C₂-C₆ alkenyl, an optionally substituted C₂-C₆ alkynyl, an optionally substituted C₃-C₅ cycloalkyl, an optionally substituted heterocycle selected from pyrrolidine, pyrrolidinone, piperidine or morpholine, an optionally substituted phenyl, an optionally substituted 1,3-benzodioxole, an optionally substituted 2,3-dihydro-1,4-benzodioxine, or an optionally substituted heteroaryl selected from pyridine, pyrimidine, pyridazine, pyrazine, pyrazole, isoxazole, isothiazole, imidazole, oxazole, or thiazole; wherein the optionally substituted C₁-C₆ alkyl, C₂-C₆ alkenyl, or C₂-C₆ alkynyl is each optionally substituted with a —CN, —OH, oxetanyl, C₁-C₃ alkoxy, or —CONR₁₁R₁₁; the optionally substituted C₃-C₅ cycloalkyl, phenyl, 1,3-benzodioxole, 2,3-dihydro-1,4-benzodioxine, heterocycle or heteroaryl is each optionally substituted with one to three substituents each independently selected from halogen, C₁-C₃ alkyl, C₁-C₃ haloalkyl, C₁-C₃ alkoxy, C₁-C₃ haloalkoxy, —SO₂R₁₁, —NR₁₁R₁₁, —OH or —CN; or
R₂ is an optionally substituted bicyclic ring selected from 1,3-benzodioxole, 2,3-dihydro-1,4-benzodioxine, isoindolin-1-one, indolin-2-one, benzo[d]oxazol-2(3H)-one, 1,3-dihydro-2H-pyrrolo[2,3-b]pyridin-2-one, or 2,3-dihydro-[1,4]dioxino[2,3-b]pyridine, or an optionally substituted bicyclic heteroaryl of 8 to 10 ring atoms containing 1, 2, 3, 4, or 5 ring heteroatoms independently selected from N, O, or S; wherein the optionally substituted bicyclic ring is optionally substituted with one to three substituents each independently selected from halogen and C₁-C₆ alkyl; the optionally substituted bicyclic heteroaryl is optionally substituted with one to three substituents each independently selected from —CN, halogen, C₁-C₆ haloalkyl, C₁-C₆ alkoxy, C₁-C₆ haloalkoxy, —SO₂R₁₁, —CO₂C₁-C₃ alkyl, —CONR₁₁R₁₁, —NR₁₁R₁₁, —NR₁₁CO₂R₁₁, —OH, an optionally substituted C₁-C₆ alkyl, an optionally substituted C₂-C₆ alkenyl, an optionally substituted C₂-C₆ alkynyl, an optionally substituted C₃-C₅ cycloalkyl, an optionally substituted heterocycle selected from pyrrolidine, pyrrolidinone, piperidine or morpholine, an optionally substituted phenyl, an optionally substituted 1,3-benzodioxole, an optionally substituted 2,3-dihydro-1,4-benzodioxine, or an optionally substituted heteroaryl selected from pyridine, pyrimidine, pyridazine, pyrazine, pyrazole, isoxazole, isothiazole, imidazole, oxazole, or thiazole; wherein the optionally substituted C₁-C₆ alkyl, C₂-C₆ alkenyl, or C₂-C₆ alkynyl is each optionally substituted with a —CN, —OH, oxetanyl, C₁-C₃ alkoxy, —CONR₁₁R₁₁ or phenyl; the optionally substituted C₃-C₅ cycloalkyl, phenyl, 1,3-benzodioxole, 2,3-dihydro-1,4-benzodioxine, heterocycle or heteroaryl is each optionally substituted with one to three substituents each independently selected from halogen, C₁-C₃ alkyl, C₁-C₃ haloalkyl, C₁-C₃ alkoxy, C₁-C₃ haloalkoxy, —SO₂R₁₁, —NR₁₁R₁₁, —OH or —CN; or
R₂ is a cyclopropyl, cyclobutyl, cyclopentyl, or cyclohexyl, each optionally substituted with one to three R₁₀ substituents, or R₂ is a group of the formula:

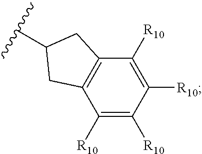

R₃ is —H, halogen, —CN, —N(H)(C₁-C₃ alkyl), —N(C₁-C₃ alkyl)₂, —N(H)(CH₂CH₂CO₂H), —CO—C₁-C₃ alkyl, C₁-C₆ alkyl, C₁-C₆ haloalkyl, C₁-C₆ hydroxyalkyl, C₃-C₅ cycloalkyl, an optionally substituted heterocycle of 3 to 5 ring atoms containing 1, 2, or 3 ring heteroatoms independently selected from N, O, or S, or an optionally substituted heteroaryl of 5 or 6 ring atoms containing 1, 2, or 3 ring heteroatoms independently selected from N, O, or S; wherein the optionally substituted heterocycle or heteroaryl is each optionally substituted with one to three substituents each independently selected from halogen, C₁-C₃ alkyl, or C₁-C₃ haloalkyl; each of R₄, R₅ and R₆ is independently —H, halogen, C₁-C₆ alkyl or C₁-C₆ haloalkyl;
R₇ is —CN, C₁-C₆ alkyl or C₁-C₆ haloalkyl;
R₈ is —H or C₁-C₆ alkyl;
each R₁₀ is independently —H, —CN, halogen, C₁-C₆ haloalkyl, C₁-C₆ alkoxy, C₁-C₆ haloalkoxy, —SO₂R₁₁, —SONR₁₁R₁₁, —CO₂H, —CO₂C₁-C₃ alkyl, —CONR₁₁R₁₂, —NR₁₁R₁₁, —NR₁₁—CO₂R₁₁, —OH, an optionally substituted C₁-C₆ alkyl, an optionally substituted C₂-C₆ alkenyl, an optionally substituted C₂-C₆ alkynyl, an optionally substituted C₃-C₅ cycloalkyl, an optionally substituted heterocycle selected from pyrrolidine, pyrrolidinone, piperidine or morpholine, an optionally substituted phenyl, an optionally substituted 1,3-benzodioxole, an optionally substituted 2,3-dihydro-1,4-benzodioxine, or an optionally substituted heteroaryl selected from pyrazole, isoxazole, isothiazole, imidazole, oxazole, thiazole or pyridine, or a group of the formula:

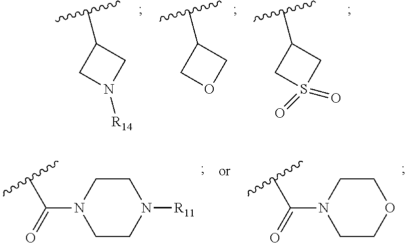

wherein the optionally substituted C₁-C₆ alkyl, C₂-C₆ alkenyl, or C₂-C₆ alkynyl is each optionally substituted with a —CN, —OH, oxetanyl, C₁-C₃ alkoxy, or —CONR₁₁R₁₁; the optionally substituted C₃-C₅ cycloalkyl, phenyl, 1,3-benzodioxole, 2,3-dihydro-1,4-benzodioxine, heterocycle or heteroaryl is each optionally substituted with one to three substituents each independently selected from halogen, C₁-C₃ alkyl, C₁-C₃ haloalkyl, C₁-C₃ alkoxy, C₁-C₃ haloalkoxy, —SO₂R₁₁, —NR₁₁R₁₁, —OH or —CN;
each R₁₁ is independently —H or C₁-C₃ alkyl; and
each R₁₂ is independently —H, optionally substituted C₁-C₃ alkyl, C₃-C₆ cycloalkyl, —SO₂C₁-C₃ alkyl, —SO₂C₁-C₃ haloalkyl, —SO₂NR₁₁R₁₁, —NR₁₁R₁₁, —OR₁₁, —O—CH₂—CH(OH)—CH₂OH, —CN, oxetane, tetrahydrofuran, an aryl, a 5-member heteroaryl optionally substituted with a methyl, a 6-member heteroaryl, or a group of the formula

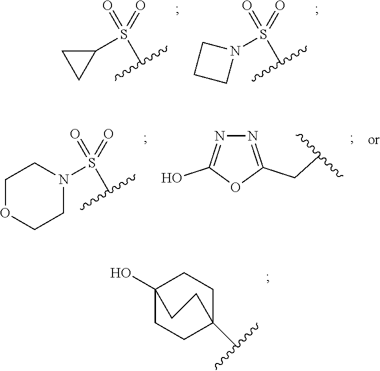 [SMILES: *S(=O)(=O)C1CC1 ; *S(=O)(=O)N1CCC1 ; *S(=O)(=O)N1CCOCC1 ; *Cc1nnc(O)o1 ; *C12CCC(O)(CC1)CC2]

wherein the optionally substituted C₁-C₃ alkyl is optionally substituted with —OH, a C₃-C₆ cycloalkyl, oxetane, tetrahydrofuran, an aryl, a 5-member heteroaryl, a 6-member heteroaryl or an indole. 3. The compound as defined in aspect 1 or 2, or pharmaceutically acceptable salt thereof, wherein R′ is C₁-C₃ haloalkyl, C₁-C₃ alkoxy, —NO₂, —N(R₁₁)—CO₂C₁-C₃ alkyl, —N(R₁₁)—SO₂C₁-C₃ alkyl, —SO₂NR₁₁R₁₁, —SO₂N(R₁₁)—CO—C₁-C₃ alkyl, —C(═N—OH)—NH₂, —CN, —CO₂C₁-C₃ alkyl, —CONR₁₁R₁₂, —CON(R)—(CH₂)ₙ—R₁₃, or a group of the formula

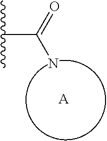

wherein ring A is pyrrolidine optionally substituted with a —CN; or
R′ is selected from oxetane, pyrrolidine, tetrahydrofuran, pyrrole, furan, thiophene, pyrazole, imidazole, isoxazole, oxazole, isothiazole, thiazole, triazole, oxadiazole, thiadiazole, tetrazole, pyridine, pyridazine, pyrimidine, pyrazine, or triazine; each of which is optionally substituted with one to three substituents independently selected from oxo, —OH, —NR₁₁R₁₁, —N(R₁₁)—CO—R₁₁, —N(R₁₁)—CN, —OR₁₁, —CN, C₁-C₆ haloalkyl or C₁-C₆ alkyl optionally substituted with an aryl, a 5-member heteroaryl or a 6-member heteroaryl.
   4. The compound as defined in any of aspects 1-3, or pharmaceutically acceptable salt thereof, wherein R and R₁ together with the nitrogen to which they are attached form an optionally substituted bicyclic ring selected from dihydrobenzimidazolone, dihydroindazolone, indolinone or quinazolinone; wherein the optionally substituted bicyclic ring is optionally substituted with one to three substituents each independently selected from oxo, —CN, halogen, C₁-C₆ alkyl, C₁-C₆ haloalkyl, —OH or C₁-C₆ alkoxy; or
R is —H or C₁-C₃ alkyl; and
R₁ is an optionally substituted bicyclic ring selected from isobenzofuranone, benzofuranone, isoindolinone, indolinone, quinazolinone, or benzothiazolone; wherein the optionally substituted bicyclic ring is optionally substituted with one to three substituents each independently selected from oxo, —CN, halogen, C₁-C₆ alkyl, C₁-C₆ haloalkyl, —OH or C₁-C₆ alkoxy; or
   R₁ is a group of the formula:

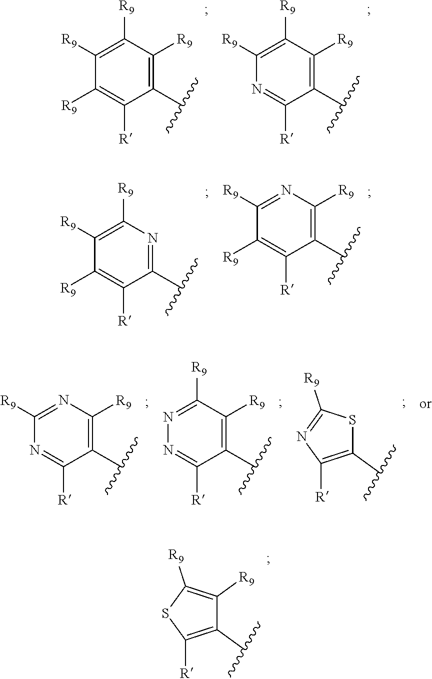

and
R′ is C₁-C₃ haloalkyl, C₁-C₃ alkoxy, —NO₂, —N(R₁₁)—CO₂C₁-C₃ alkyl, —N(R₁₁)—SO₂C₁-C₃ alkyl, —SO₂NR₁₁R₁₁, —SO₂N(R₁₁)—CO—C₁-C₃ alkyl, —C(═N—OH)—NH₂, —CN, —CO₂C₁-C₃ alkyl, —CONR₁₁R₁₂, —CON(R)—(CH₂)ₙ—R₁₃, or a group of the formula

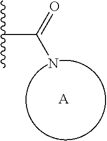

wherein ring A is pyrrolidine optionally substituted with a —CN; or
R′ is selected from oxetane, pyrrolidine, tetrahydrofuran, pyrrole, furan, thiophene, pyrazole, imidazole, isoxazole, oxazole, isothiazole, thiazole, triazole, oxadiazole, thiadiazole, tetrazole, pyridine, pyridazine, pyrimidine, pyrazine, or triazine; each of which is optionally substituted with one to three substituents independently selected from oxo, —OH, —NR₁₁R₁₁, —N(R₁₁)—CO—R₁₁, —N(R₁₁)—CN, —OR₁₁, —CN, C₁-C₆ haloalkyl or C₁-C₆ alkyl optionally substituted with an aryl, a 5-member heteroaryl or a 6-member heteroaryl.
   5. The compound as defined in aspect 1 or 2, or pharmaceutically acceptable salt thereof, wherein R′ is hydrogen, halogen, C₁-C₃ alkyl, —CH(OH)—CH₂OH, —CO—CH₂OH or C₃-C₆ cycloalkyl.
   6. The compound as defined in aspect 1, 2 or 5, or pharmaceutically acceptable salt thereof, wherein
R is —H or C₁-C₃ alkyl;
   R₁ is a group of the formula:

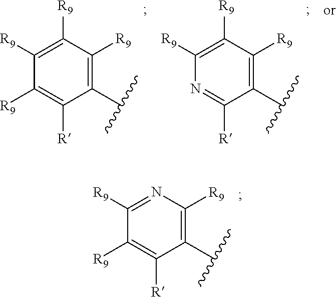

R₂ is a group of the formula:

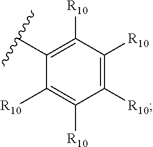

or
R₂ is a group of the formula:

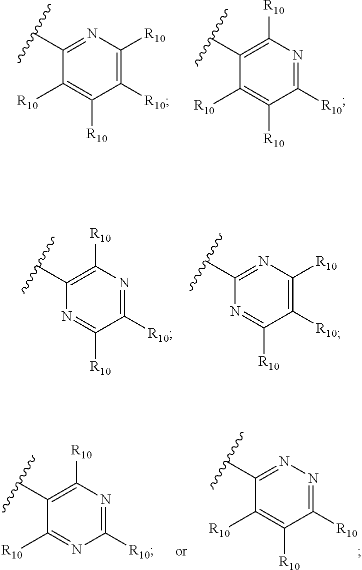

or
R₂ is an optionally substituted 5-member ring heteroaryl selected from pyrrole, furan, thiophene, pyrazole, isoxazole, isothiazole, imidazole, oxazole, thiazole, triazole, tetrazole, oxadiazole, and thiadiazole; wherein the optionally substituted 5-member ring heteroaryl is optionally substituted with one to three substituents each independently selected from —CN, halogen, C₁-C₆ haloalkyl, C₁-C₆ alkoxy, C₁-C₆ haloalkoxy, —SO₂R₁₁, —CO₂C₁-C₃ alkyl, —CONR₁₁R₁₁, —OH, —NR₁₁R₁₁, —NR₁₁CO₂R₁₁, an optionally substituted C₁-C₆ alkyl, an optionally substituted C₂-C₆ alkenyl, an optionally substituted C₂-C₆ alkynyl, an optionally substituted C₃-C₅ cycloalkyl, an optionally substituted heterocycle selected from pyrrolidine, pyrrolidinone, piperidine or morpholine, an optionally substituted phenyl, an optionally substituted 1,3-benzodioxole, an optionally substituted 2,3-dihydro-1,4-benzodioxine, or an optionally substituted heteroaryl selected from pyridine, pyrimidine, pyridazine, pyrazine, pyrazole, isoxazole, isothiazole, imidazole, oxazole, or thiazole; wherein the optionally substituted C₁-C₆ alkyl, C₂-C₆ alkenyl, or C₂-C₆ alkynyl is each optionally substituted with a —CN, —OH, oxetanyl, C₁-C₃ alkoxy, or —CONR₁₁R₁₁; the optionally substituted C₃-C₅ cycloalkyl, phenyl, 1,3-benzodioxole, 2,3-dihydro-1,4-benzodioxine, heterocycle or heteroaryl is each optionally substituted with one to three substituents each independently selected from halogen, C₁-C₃ alkyl, C₁-C₃ haloalkyl, C₁-C₃ alkoxy, C₁-C₃ haloalkoxy, —SO₂R₁₁, —NR₁₁R₁₁, —OH or —CN; or
R₂ is an optionally substituted bicyclic ring selected from 1,3-benzodioxole, 2,3-dihydro-1,4-benzodioxine, isoindolin-1-one, indolin-2-one, benzo[d]oxazol-2(3H)-one, 1,3-dihydro-2H-pyrrolo[2,3-b]pyridine-2-one, or 2,3-dihydro-[1,4]dioxino[2,3-b]pyridine, or an optionally substituted bicyclic heteroaryl of 8 to 10 ring atoms containing 1, 2, 3, 4, or 5 ring heteroatoms independently selected from N, O, or S; wherein the optionally substituted bicyclic ring is optionally substituted with one to three substituents each independently selected from halogen and C₁-C₆ alkyl; the optionally substituted bicyclic heteroaryl is optionally substituted with one to three substituents each independently selected from —CN, halogen, C₁-C₆ haloalkyl, C₁-C₆ alkoxy, C₁-C₆ haloalkoxy, —SO₂R₁₁, —CO₂C₁-C₃ alkyl, —CONR₁₁R₁₁, —NR₁₁R₁₁, —NR₁₁CO₂R₁₁, —OH, an optionally substituted C₁-C₆ alkyl, an optionally substituted C₂-C₆ alkenyl, an optionally substituted C₂-C₆ alkynyl, an optionally substituted C₃-C₅ cycloalkyl, an optionally substituted heterocycle selected from pyrrolidine, pyrrolidinone, piperidine or morpholine, an optionally substituted phenyl, an optionally substituted 1,3-benzodioxole, an optionally substituted 2,3-dihydro-1,4-benzodioxine, or an optionally substituted heteroaryl selected from pyridine, pyrimidine, pyridazine, pyrazine, pyrazole, isoxazole, isothiazole, imidazole, oxazole, or thiazole; wherein the optionally substituted C₁-C₆ alkyl, C₂-C₆ alkenyl, or C₂-C₆ alkynyl is each optionally substituted with a —CN, —OH, oxetanyl, C₁-C₃ alkoxy, —CONR₁₁R₁₁ or phenyl; the optionally substituted C₃-C₅ cycloalkyl, phenyl, 1,3-benzodioxole, 2,3-dihydro-1,4-benzodioxine, heterocycle or heteroaryl is each optionally substituted with one to three substituents each independently selected from halogen, C₁-C₃ alkyl, C₁-C₃ haloalkyl, C₁-C₃ alkoxy, C₁-C₃ haloalkoxy, —SO₂R₁₁, —NR₁₁R₁₁, —OH or —CN; or
R₂ is a cyclopropyl, cyclobutyl, cyclopentyl, or cyclohexyl, each optionally substituted with one to three R₁₀ substituents, or R₂ is a group of the formula:

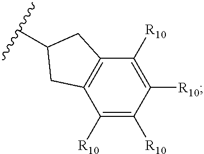

and
each R₁₀ is independently —H, —CN, halogen, C₁-C₆ haloalkyl, C₁-C₆ alkoxy, C₁-C₆ haloalkoxy, —SO₂R₁₁, —CO₂C₁-C₃ alkyl, —CONR₁₁R₁₁, —NR₁₁R₁₁, —NR₁₁—CO₂R₁₁, —OH, an optionally substituted C₁-C₆ alkyl, an optionally substituted C₂-C₆ alkenyl, an optionally substituted C₂-C₆ alkynyl, an optionally substituted C₃-C₅ cycloalkyl, an optionally substituted heterocycle selected from pyrrolidine, pyrrolidinone, piperidine or morpholine, an optionally substituted phenyl, an optionally substituted 1,3-benzodioxole, an optionally substituted 2,3-dihydro-1,4-benzodioxine, or an optionally substituted heteroaryl selected from pyrazole, isoxazole, isothiazole, imidazole, oxazole, thiazole or pyridine; wherein the optionally substituted C₁-C₆ alkyl, C₂-C₆ alkenyl, or C₂-C₆ alkynyl is each optionally substituted with a —CN, —OH, oxetanyl, C₁-C₃ alkoxy, —CO₂C₁-C₃ alkyl, —CONR₁₁R₁₁, —NR₁₁R₁₁ or phenyl; the optionally substituted C₃-C₅ cycloalkyl, phenyl, 1,3-benzodioxole, 2,3-dihydro-1,4-benzodioxine, heterocycle or heteroaryl is each optionally substituted with one to three substituents each independently selected from halogen, C₁-C₃ alkyl, C₁-C₃ haloalkyl, C₁-C₃ alkoxy, C₁-C₃ haloalkoxy, —SO₂R₁₁, —NR₁₁R₁₁, —OH or —CN.
   7. The compound as defined in any one of aspects 1-6, or pharmaceutically acceptable salt thereof, wherein R₈ is —H
   8. The compound as defined in any one of aspects 1-7, or pharmaceutically acceptable salt thereof, having the Formula:

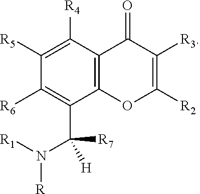

9. The compound as defined in any one of aspects 1-8, or pharmaceutically acceptable salt thereof, wherein R and R₁ together with the nitrogen to which they are attached form an optionally substituted bicyclic ring selected from dihydrobenzimidazolone, dihydroindazolone, indolinone or quinazolinone; wherein the optionally substituted bicyclic ring is optionally substituted with one to three substituents each independently selected from oxo, —CN, halogen, C₁-C₆ alkyl, C₁-C₆ haloalkyl, —OH or C₁-C₆ alkoxy.
10. The compound as defined in aspect 9, or pharmaceutically acceptable salt thereof, wherein R and R₁ together with the nitrogen to which they are attached form an optionally substituted bicyclic ring of the formula

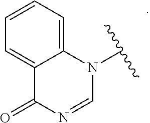 [SMILES: *n1cnc(=O)c2ccccc21]

11. The compound as defined in any one of aspects 1-8, or pharmaceutically acceptable salt thereof, wherein R is —H.
12. The compound as defined in any one of aspects 1-11, or pharmaceutically acceptable salt thereof, wherein R₄ is —H or halogen.
13. The compound as defined in any one of aspects 1-12, or pharmaceutically acceptable salt thereof, wherein R₄ is —H.
14. The compound as defined in any one of aspects 1-8 or 11-13, or pharmaceutically acceptable salt thereof having the Formula

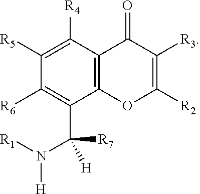

15. The compound as defined in any one of aspects 1-14, or pharmaceutically acceptable salt thereof, wherein R₃ is —H, —CN, C₁-C₃ alkyl, or C₁-C₃ haloalkyl.
16. The compound as defined in any one of aspects 1-15, or pharmaceutically acceptable salt thereof, wherein R₃ is —H, —CN, methyl, or trifluoromethyl.
17. The compound as defined in any one of aspects 1-16, or pharmaceutically acceptable salt thereof, wherein R₃ is —H or methyl.
18. The compound as defined in any one of aspects 1-16, or pharmaceutically acceptable salt thereof, wherein R₅ is —H, halogen, C₁-C₃ alkyl or C₁-C₃ haloalkyl.
19. The compound as defined in any one of aspects 1-18, or pharmaceutically acceptable salt thereof, wherein R₅ is —H, halogen, methyl, or trifluoromethyl.
20. The compound as defined in any one of aspects 1-19, or pharmaceutically acceptable salt thereof, wherein R_{b} is —H or halogen.
21. The compound as defined in any one of aspects 1-20, or pharmaceutically acceptable salt thereof, wherein R₇ is —CN, C₁-C₃ alkyl or C₁-C₃ haloalkyl.
22. The compound as defined in any one of aspects 1-21, or pharmaceutically acceptable salt thereof, wherein R₇ is —CN, methyl or trifluoromethyl.
23. The compound as defined in any one of aspects 1-22, or pharmaceutically acceptable salt thereof, wherein R₇ is methyl.
24. The compound as defined in any one of aspects 1-8 or 11-23, or pharmaceutically acceptable salt thereof, wherein R₁ is an optionally substituted bicyclic ring selected from isobenzofuranone, benzofuranone, isoindolinone, indolinone, quinazolinone, or benzothiazolone; wherein the optionally substituted bicyclic ring is optionally substituted with one to three substituents each independently selected from oxo, —CN, halogen, C₁-C₆ alkyl, C₁-C₆ haloalkyl, —OH or C₁-C₆ alkoxy.
25. The compound as defined in any one of aspects 1-8 or 11-23, or pharmaceutically acceptable salt thereof, wherein R₁ is a group of the formula

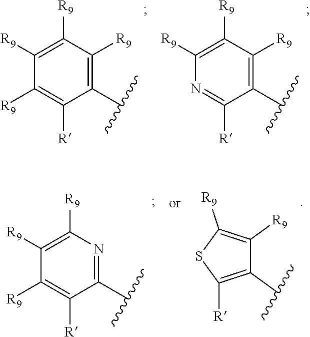

26. The compound of any one of claims 1-8 or 11-23, or pharmaceutically acceptable salt thereof, wherein R₁ is a group of the formula

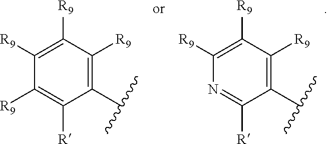

27. The compound as defined in any one of aspects 1-8, 11-23, 25 or 26, or pharmaceutically acceptable salt thereof, wherein R₉ is independently —H, halogen, C₁-C₃ alkyl, or C₁-C₃ alkoxy.
28. The compound as defined in any one of aspects 1-8, 11-23, 25, or 26, or pharmaceutically acceptable salt thereof, wherein each R₉ is independently —H, halogen, C₁-C₃ alkyl, or C₃-C₅ cycloalkyl.
29. The compound as defined in any one of aspects 1-4, 7-8, 11-23, or 25-28, or pharmaceutically acceptable salt thereof, wherein R′ is C₁-C₃ haloalkyl, C₁-C₃ alkoxy, —NO₂, —N(R₁₁)—CO₂C₁-C₃ alkyl, —N(R₁₁)—SO₂C₁-C₃ alkyl, —SO₂NR₁₁R₁₁, —SO₂N(R₁₁)—CO—C₁-C₃ alkyl, —C(═N—OH)—NH₂, —CN, —CONR₁₁R₁₂, —CON(R₁₁)—(CH₂)ₙ—R₁₃, or a group of the formula

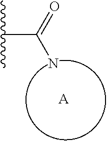

wherein ring A is pyrrolidine optionally substituted with a —CN; or
R′ is selected from oxetane, pyrrolidine, tetrahydrofuran, pyrrole, furan, thiophene, pyrazole, imidazole, isoxazole, oxazole, isothiazole, thiazole, triazole, oxadiazole, thiadiazole, tetrazole, pyridine, pyridazine, pyrimidine, pyrazine, or triazine; each of which is optionally substituted with one to three substituents independently selected from oxo, —NR₁₁R₁₁, —N(R₁₁)—CO—R₁₁, —N(R₁₁)—CN, —OR₁₁, —CN, C₁-C₆ haloalkyl or C₁-C₆ alkyl optionally substituted with phenyl.
   30. The compound as defined in aspect 29, or pharmaceutically acceptable salt thereof, wherein R′ is C₁-C₃ haloalkyl, C₁-C₃ alkoxy, —NO₂, —N(R₁₁)—CO₂C₁-C₃ alkyl, —N(R₁₁)—SO₂C₁-C₃ alkyl, —SO₂NR₁₁R₁₁, —SO₂N(R₁₁)—CO—C₁-C₃ alkyl, —C(═N—OH)—NH₂, —CN, —CO₂C₁-C₃ alkyl, —CONR₁₁R₁₂, —CON(R₁₁)—(CH₂)ₙ—R₁₃, or

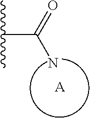

wherein ring A is pyrrolidine optionally substituted with a —CN.
   31. The compound as defined in aspect 29, or pharmaceutically acceptable salt thereof, wherein R′ is selected from oxetane, pyrrolidine, tetrahydrofuran, pyrrole, furan, thiophene, pyrazole, imidazole, isoxazole, oxazole, isothiazole, thiazole, triazole, oxadiazole, thiadiazole, tetrazole, pyridine, pyridazine, pyrimidine, pyrazine, or triazine; each of which is optionally substituted with one to three substituents independently selected from oxo, —NR₁₁R₁₁, —OR₁₁, —CN, C₁-C₆ haloalkyl or C₁-C₆ alkyl optionally substituted with phenyl.
   32. The compound as defined in any one of aspects 1-4, 7-8, 11-23, or 25-28, or pharmaceutically acceptable salt thereof, wherein R′ is pyrrole, furan, thiophene, pyrazole, isoxazole, oxazole, isothiazole, thiazole, imidazole, triazole, oxadiazole, thiadiazole, tetrazole, pyridine, pyridazine, pyrimidine, pyrazine, or triazine; each of which is optionally substituted with one to three substituents independently selected from oxo, —NR₁₁R₁₁, —OR₁₁ or C₁-C₆ alkyl optionally substituted with aryl.
   33. The compound as defined in aspect 32, or pharmaceutically acceptable salt thereof, wherein R′ is hydrogen, halogen, C₁-C₃ alkyl, or C₃-C₆ cycloalkyl.
   34. The compound as defined in aspect 32, or pharmaceutically acceptable salt thereof, wherein R′ is hydrogen, halogen, or methyl.
   35. The compound as defined in any one of aspects 1-4, 7-8, 11-23, or 25-28, or pharmaceutically acceptable salt thereof, wherein R′ is hydrogen, —Cl, —F—, —Br, methyl, difluoromethyl, trifluoromethyl, methoxy, —NO₂, —CN, or a group of the formula:

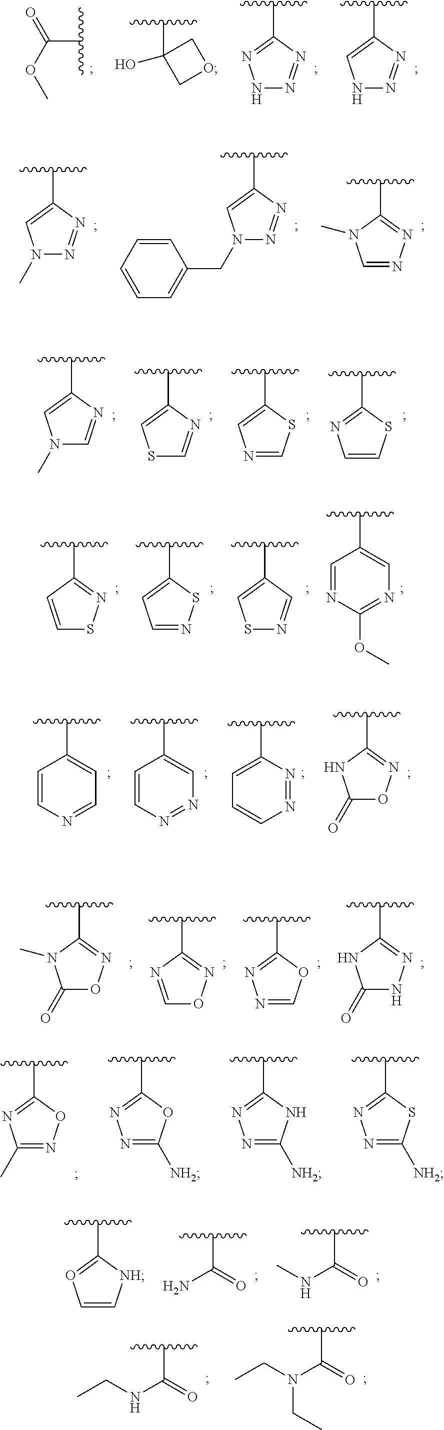

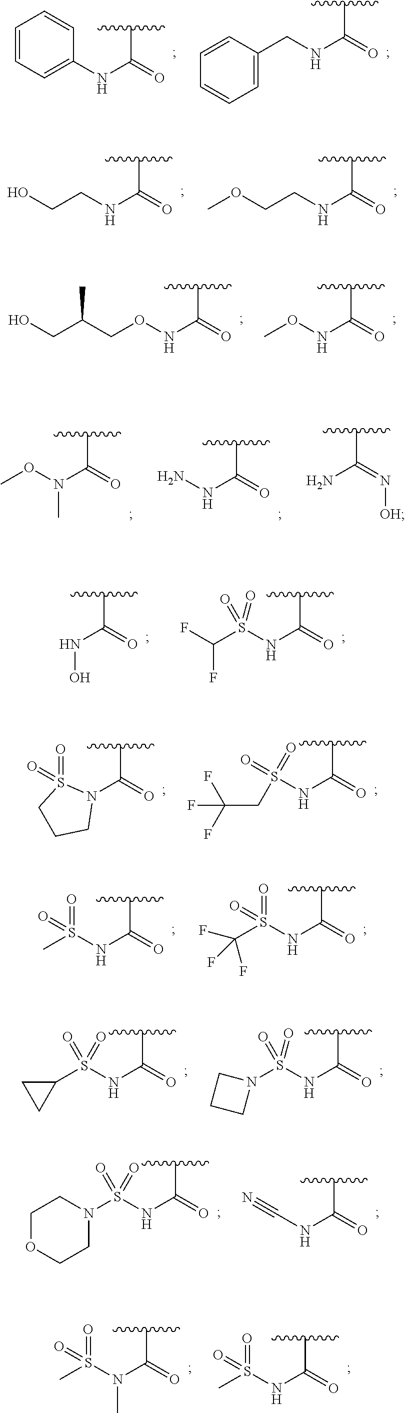

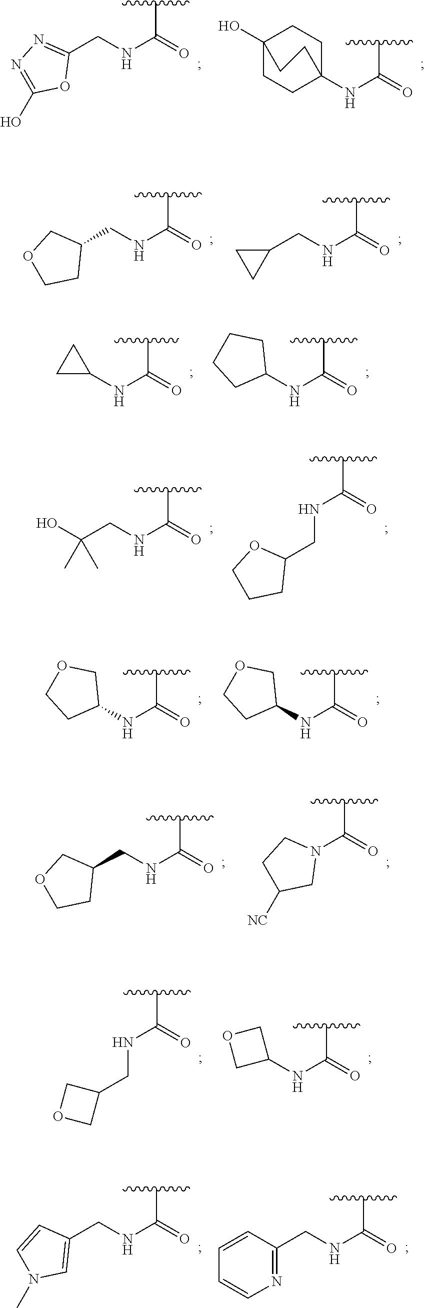

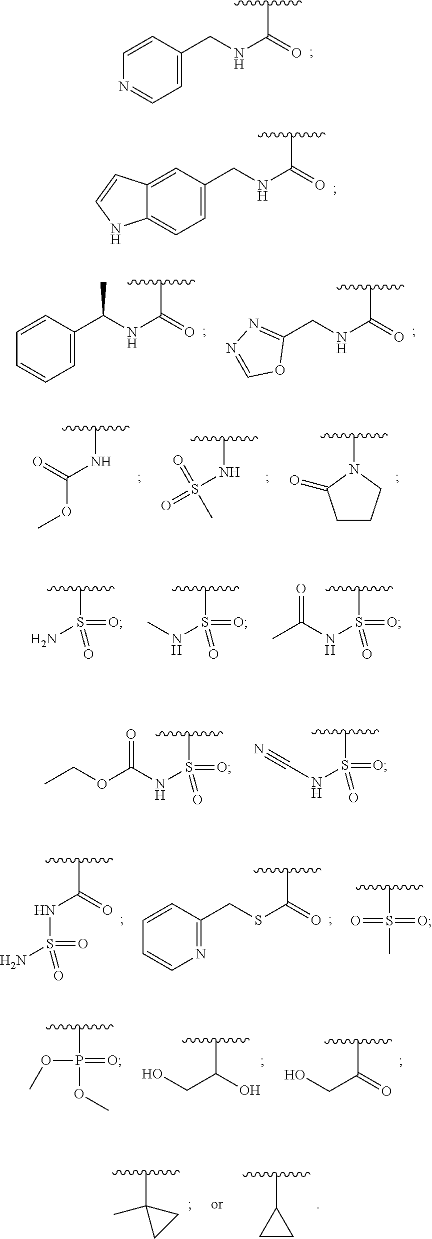

36. The compound as defined in any one of aspects 1-8 or 11-23, or pharmaceutically acceptable salt thereof, wherein R₁ is a group of the formula

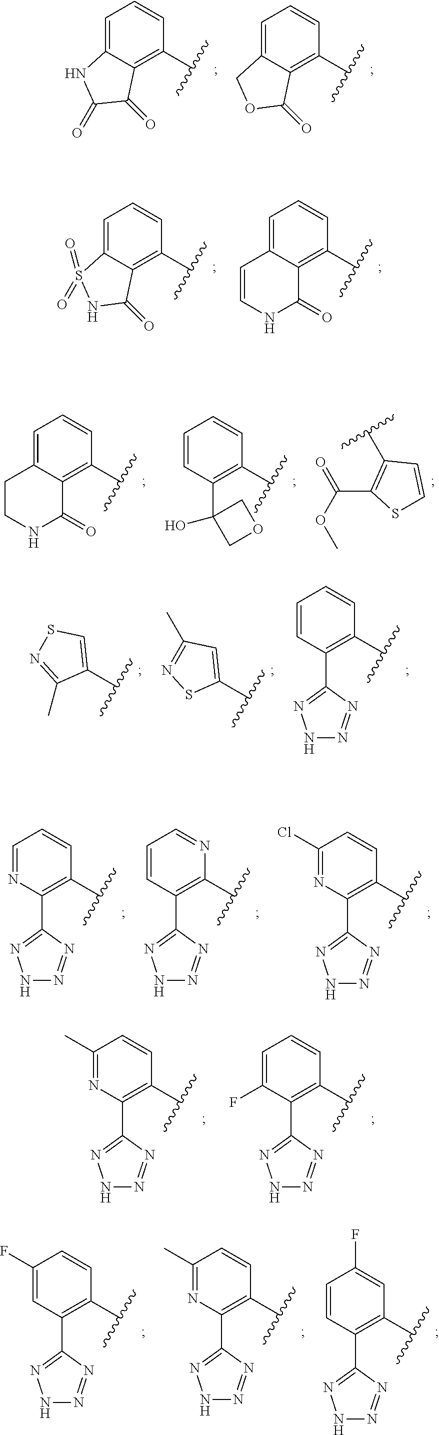

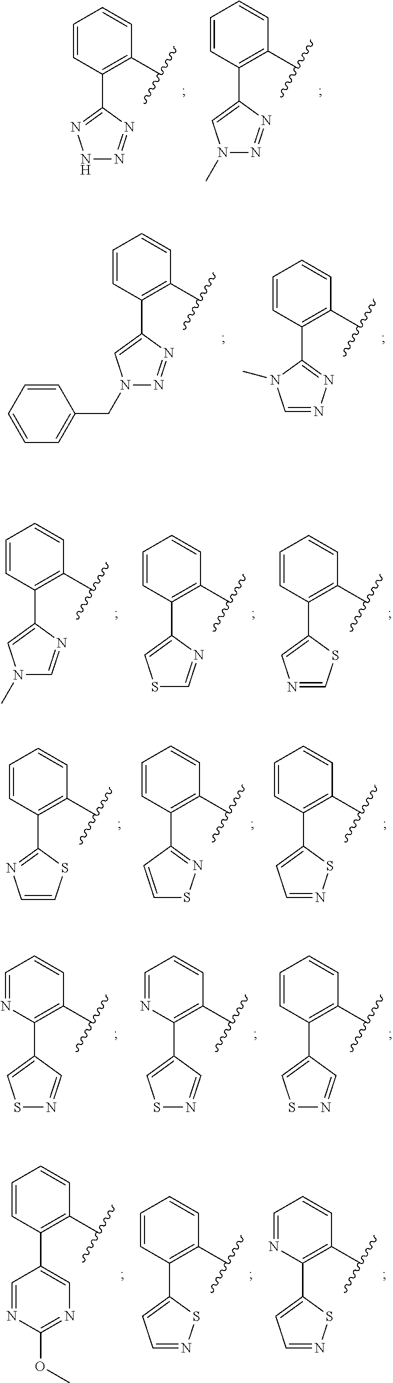

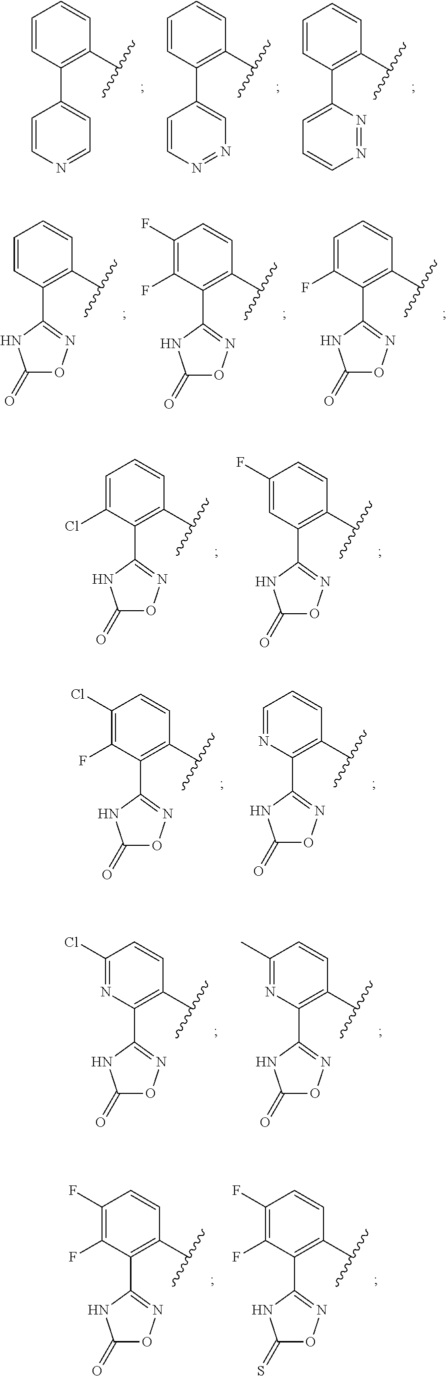

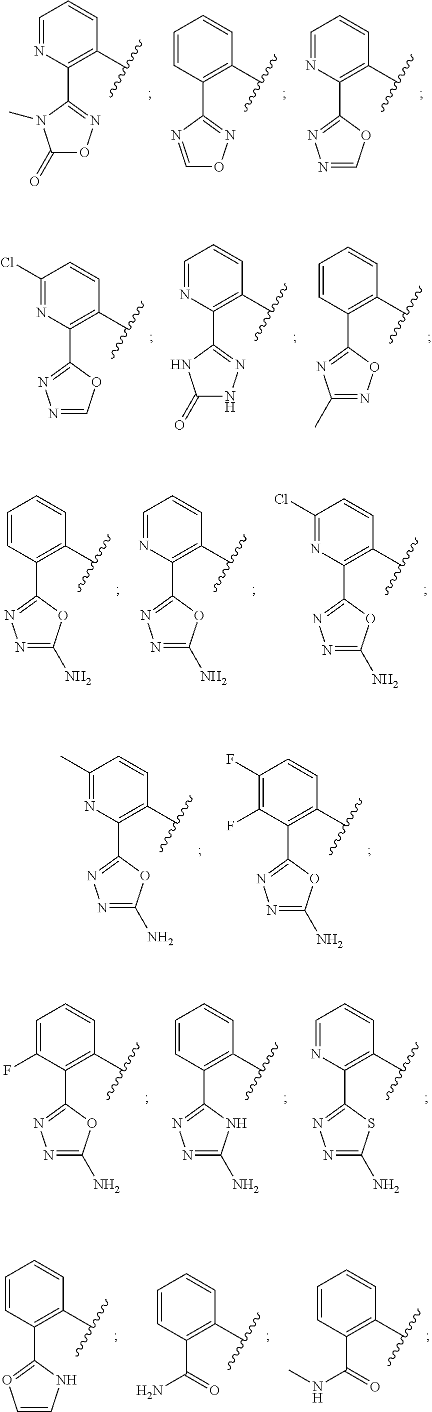

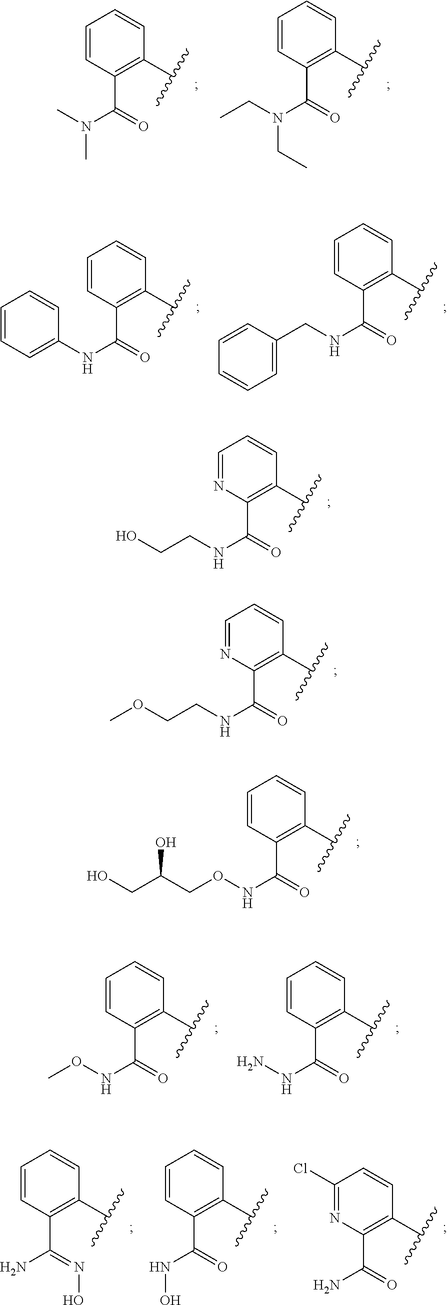

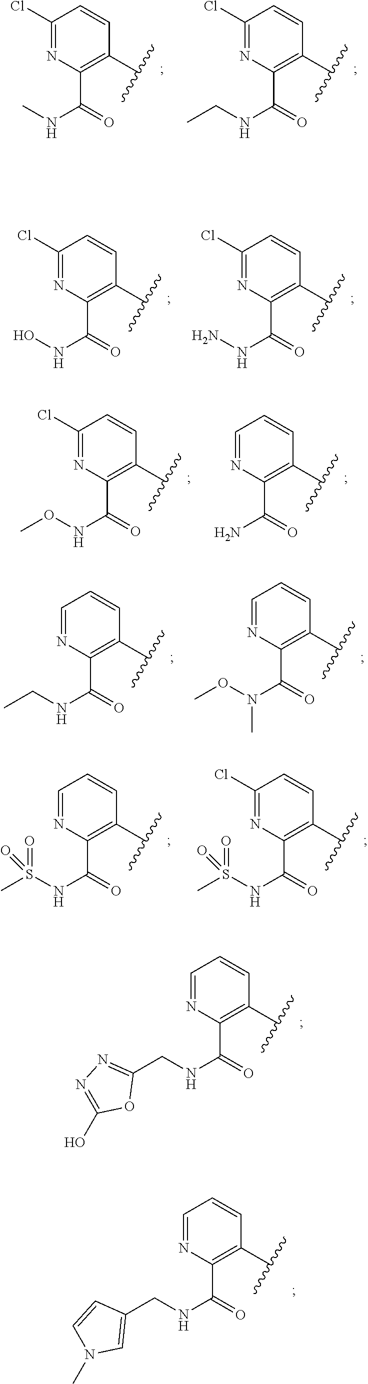

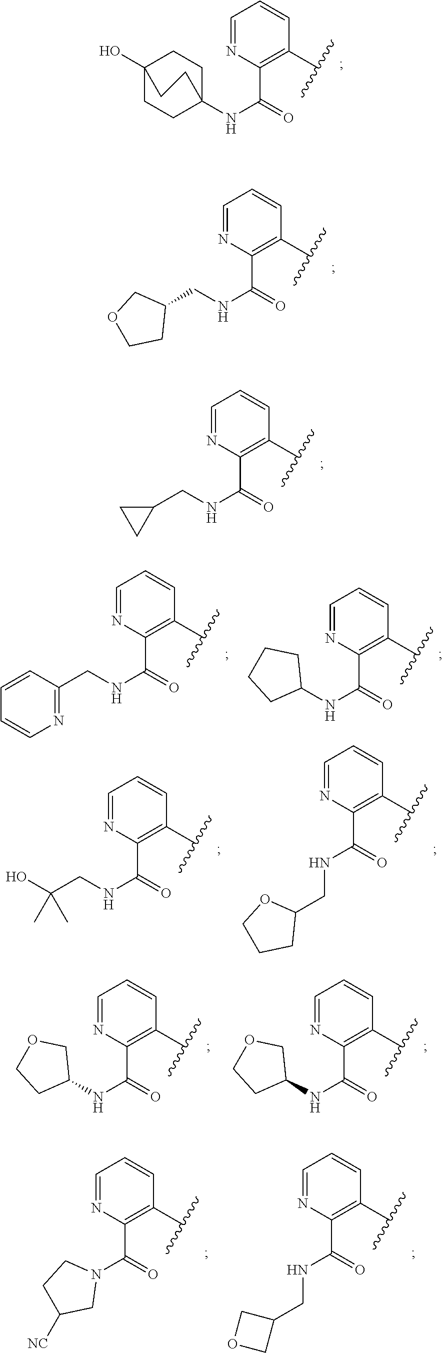

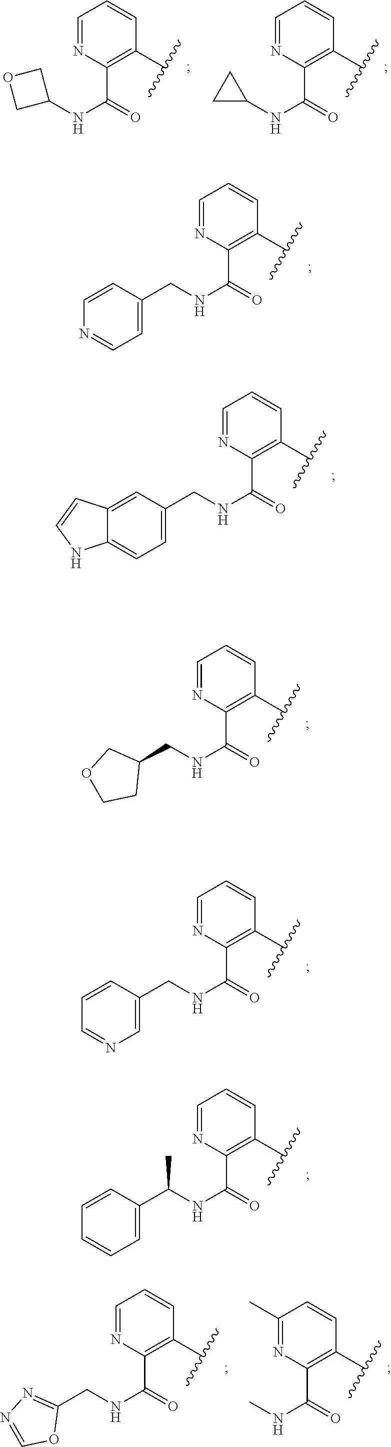

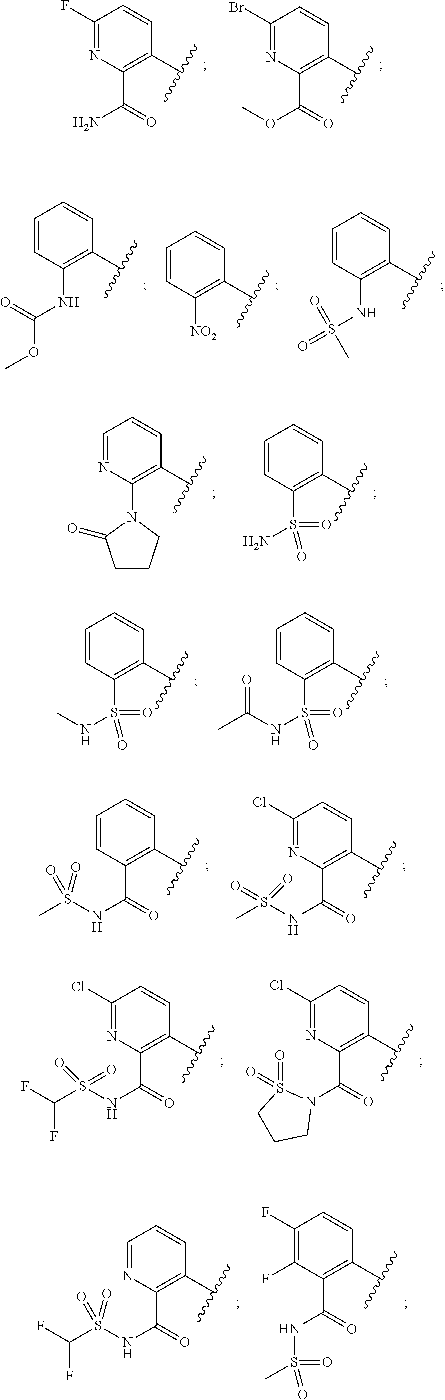

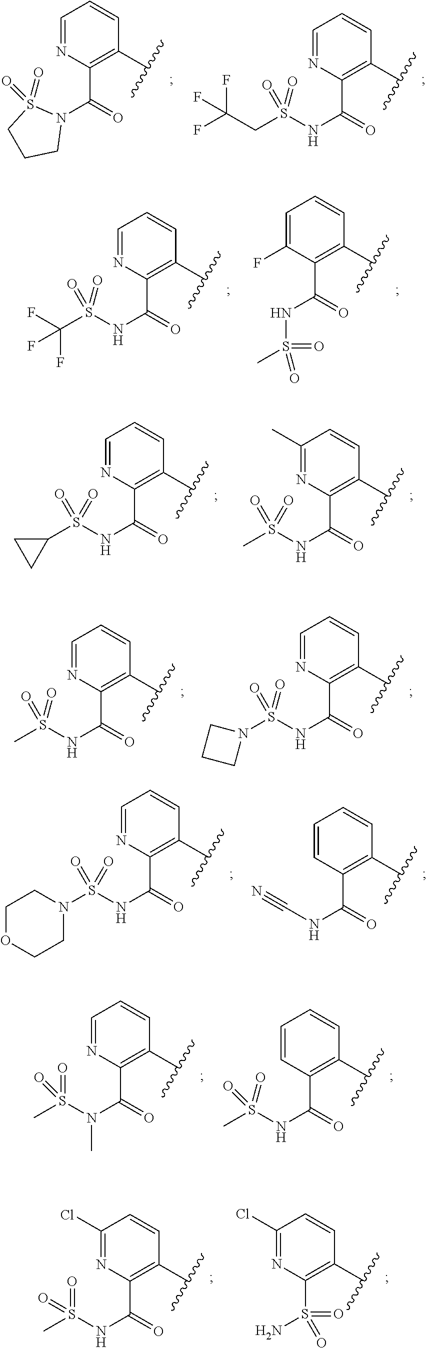

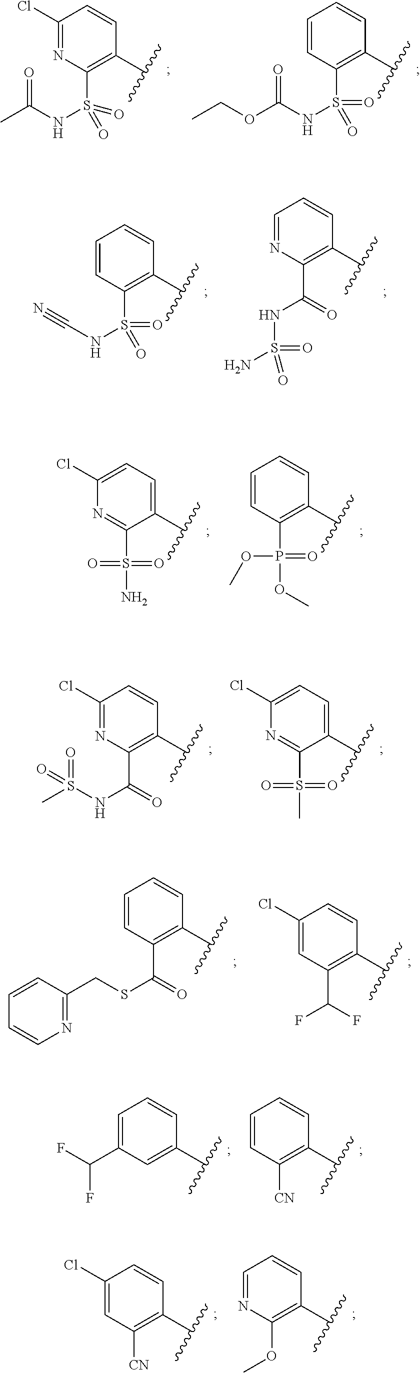

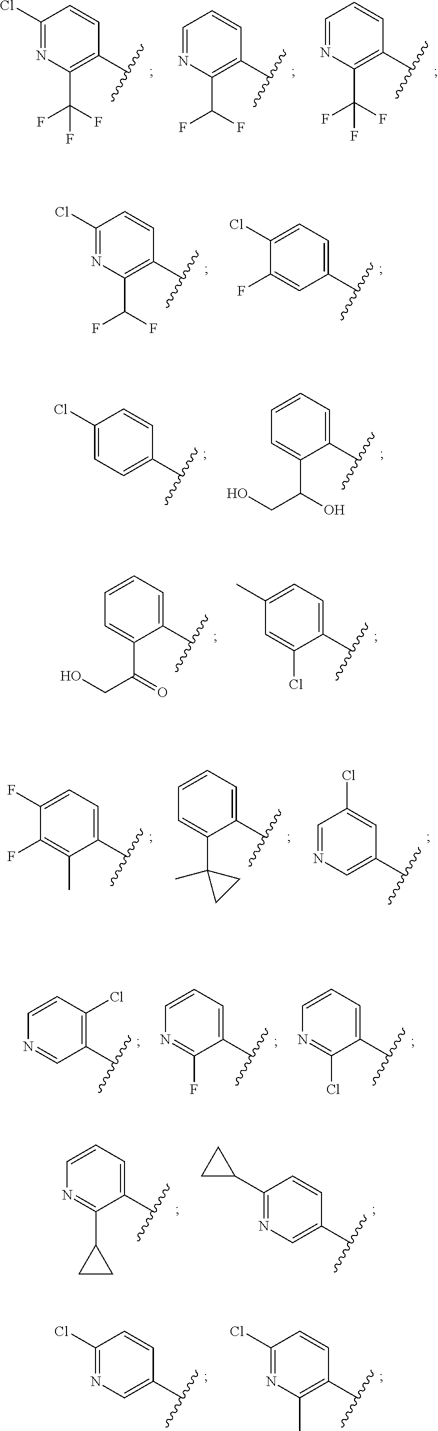

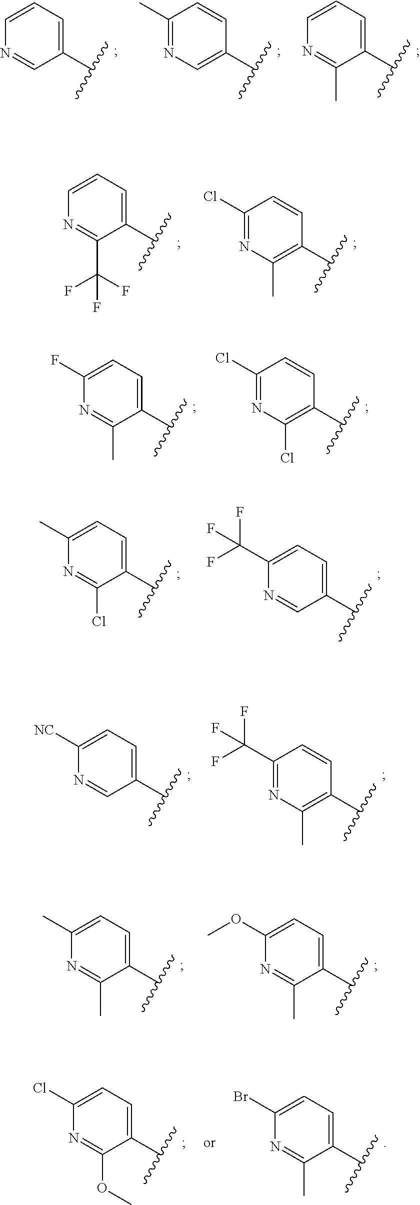

37. The compound as defined in any one of aspects 1-36, or pharmaceutically acceptable salt thereof, wherein R₂ is a group of the formula

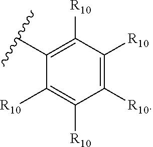

38. The compound as defined in any one of aspects 1-36, or pharmaceutically acceptable salt thereof, wherein R₂ is a group of the formula

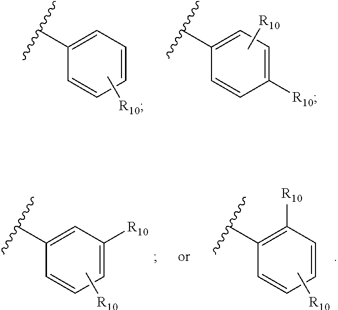

39. The compound as defined in any one of aspects 1-36, or pharmaceutically acceptable salt thereof, wherein R₂ is a group of the formula

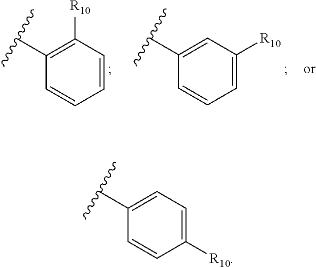

40. The compound as defined in any one of aspects 1-36, or pharmaceutically acceptable salt thereof, wherein R₂ is a group of the formula

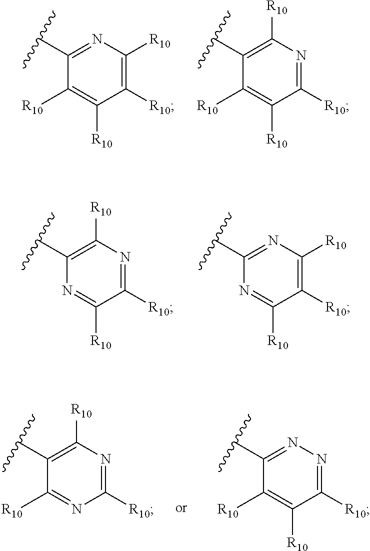

or
R₂ is an optionally substituted 5-member ring heteroaryl selected from pyrrole, furan, thiophene, pyrazole, isoxazole, isothiazole, imidazole, oxazole, thiazole, triazole, tetrazole, oxadiazole, and thiadiazole; wherein the optionally substituted 5-member ring heteroaryl is optionally substituted with one to three substituents each independently selected from —CN, halogen, C₁-C₆ haloalkyl, C₁-C₆ alkoxy, C₁-C₆ haloalkoxy, —SO₂R₁₁, —CO₂C₁-C₃ alkyl, —CONR₁₁R₁₁, —OH, —NR₁₁R₁₁, —NR₁₁CO₂R₁₁, an optionally substituted C₁-C₆ alkyl, an optionally substituted C₂-C₆ alkenyl, an optionally substituted C₂-C₆ alkynyl, an optionally substituted C₃-C₅ cycloalkyl, an optionally substituted heterocycle selected from pyrrolidine, pyrrolidinone, piperidine or morpholine, an optionally substituted phenyl, an optionally substituted 1,3-benzodioxole, an optionally substituted 2,3-dihydro-1,4-benzodioxine, or an optionally substituted heteroaryl selected from pyridine, pyrimidine, pyridazine, pyrazine, pyrazole, isoxazole, isothiazole, imidazole, oxazole, or thiazole; wherein the optionally substituted C₁-C₆ alkyl, C₂-C₆ alkenyl, or C₂-C₆ alkynyl is each optionally substituted with a —CN, —OH, oxetanyl, C₁-C₃ alkoxy, or —CONR₁₁R₁₁; the optionally substituted C₃-C₅ cycloalkyl, phenyl, 1,3-benzodioxole, 2,3-dihydro-1,4-benzodioxine, heterocycle or heteroaryl is each optionally substituted with one to three substituents each independently selected from halogen, C₁-C₃ alkyl, C₁-C₃ haloalkyl, C₁-C₃ alkoxy, C₁-C₃ haloalkoxy, —SO₂R₁₁, —NR₁₁R₁₁, —OH or —CN.
   41. The compound as defined as defined in any one of aspects 1-36, or pharmaceutically acceptable salt thereof, wherein R₂ is a group of the formula

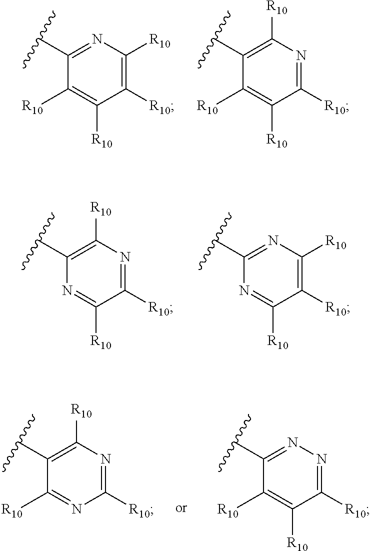

and
each R₁₀ is independently —H, —CN, halogen, C₁-C₆ haloalkyl, C₁-C₆ alkoxy, —SO₂R₁₁, an optionally substituted C₁-C₆ alkyl, an optionally substituted C₂-C₆ alkynyl, an optionally substituted C₃-C₅ cycloalkyl, or an optionally substituted heteroaryl selected from pyrazole, isoxazole, isothiazole, imidazole, oxazole, or thiazole; wherein the optionally substituted C₁-C₆ alkyl or C₂-C₆ alkynyl is optionally substituted with a —CN, —OH, or C₁-C₃ alkoxy; and the optionally substituted C₃-C₅ cycloalkyl or heteroaryl is optionally substituted with one to three substituents each independently selected from halogen, C₁-C₃ alkyl, C₁-C₃ haloalkyl, C₁-C₃ alkoxy, C₁-C₃ haloalkoxy, —NR₁₁R₁₁, —OH or —CN.
   42. The compound as defined as defined in any one of aspects 1-36, or pharmaceutically acceptable salt thereof, wherein R₂ is a group of the formula

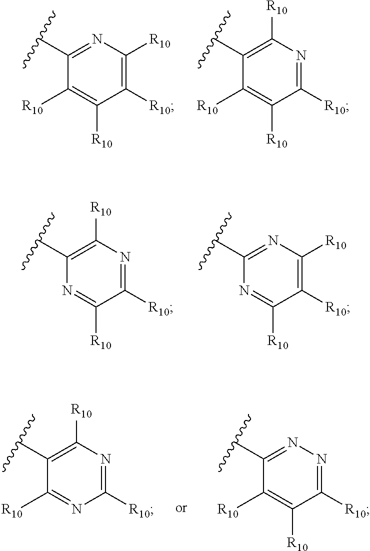

and
each R₁₀ is independently —H, —CN, halogen, C₁-C₆ haloalkyl, C₁-C₆ alkoxy, —SO₂R₁₁, a C₁-C₆ alkyl, a C₂-C₆ alkynyl optionally substituted with —OH, a C₃ cycloalkyl optionally substituted with —CN, or a heteroaryl selected from pyrazole optionally substituted with one to three substituents each independently selected from C₁-C₃ alkyl.
   43. The compound as defined as defined in any one of aspects 1-36, or pharmaceutically acceptable salt thereof, wherein R₂ is an optionally substituted 5-member ring heteroaryl selected from pyrrole, furan, thiophene, pyrazole, isoxazole, isothiazole, imidazole, oxazole, thiazole, triazole, oxadiazole, and thiadiazole; wherein the optionally substituted 5-member ring heteroaryl is optionally substituted with one to three substituents each independently selected from —CN, halogen, C₁-C₆ haloalkyl, C₁-C₆ alkoxy, C₁-C₆ haloalkoxy, —SO₂R₁₁, —CONR₁₁R₁₁, —OH, —NR₁₁R₁₁, —NR₁₁CO₂R₁₁, an optionally substituted C₁-C₆ alkyl, an optionally substituted C₂-C₆ alkenyl, an optionally substituted C₂-C₆ alkynyl, an optionally substituted C₃-C₅ cycloalkyl, an optionally substituted heterocycle selected from pyrrolidine, pyrrolidinone, piperidine or morpholine, an optionally substituted phenyl, an optionally substituted 1,3-benzodioxole, an optionally substituted 2,3-dihydro-1,4-benzodioxine, or an optionally substituted heteroaryl selected from pyridine, pyrimidine, pyridazine, pyrazine, pyrazole, isoxazole, isothiazole, imidazole, oxazole, or thiazole; wherein the optionally substituted C₁-C₆ alkyl, C₂-C₆ alkenyl, or C₂-C₆ alkynyl is each optionally substituted with a —CN, —OH, oxetanyl, or C₁-C₃ alkoxy; the optionally substituted C₃-C₅ cycloalkyl, phenyl, 1,3-benzodioxole, 2,3-dihydro-1,4-benzodioxine, heterocycle or heteroaryl is each optionally substituted with one to three substituents each independently selected from halogen, C₁-C₃ alkyl, C₁-C₃ haloalkyl, C₁-C₃ alkoxy, C₁-C₃ haloalkoxy, —SO₂R₁₁, —NR₁₁R₁₁, —OH or —CN.
   44. The compound as defined as defined in any one of aspects 1-36, or pharmaceutically acceptable salt thereof, wherein R₂ is an optionally substituted 5-member ring heteroaryl selected from pyrrole, furan, thiophene, pyrazole, isoxazole, isothiazole, imidazole, oxazole, thiazole, triazole, oxadiazole, and thiadiazole; wherein the optionally substituted 5-member ring heteroaryl is optionally substituted with one to three substituents each independently selected from C₁-C₆ haloalkyl, C₁-C₆ alkyl, an optionally substituted phenyl, an optionally substituted 1,3-benzodioxole, or an optionally substituted heteroaryl selected from pyridine or pyrimidine; wherein the optionally substituted phenyl, 1,3-benzodioxole, or heteroaryl is each optionally substituted with one to three substituents each independently selected from halogen, C₁-C₃ haloalkyl, C₁-C₃ alkoxy, C₁-C₃ haloalkoxy, —SO₂R₁₁, or —CN.
   45. The compound as defined as defined in any one of aspects 1-36, or pharmaceutically acceptable salt thereof, wherein R₂ is an optionally substituted bicyclic ring selected from 1,3-benzodioxole, 2,3-dihydro-1,4-benzodioxine, isoindolin-1-one, indolin-2-one, benzo[d]oxazol-2(3H)-one, 1,3-dihydro-2H-pyrrolo[2,3-b]pyridin-2-one, or 2,3-dihydro-[1,4]dioxino[2,3-b]pyridine, or an optionally substituted bicyclic heteroaryl of 8 to 10 ring atoms containing 1, 2, 3, 4, or 5 ring heteroatoms independently selected from N, O, or S; wherein the optionally substituted bicyclic ring is optionally substituted with one to three substituents each independently selected from halogen and C₁-C₆ alkyl; the optionally substituted bicyclic heteroaryl is optionally substituted with one to three substituents each independently selected from —CN, halogen, C₁-C₆ haloalkyl, C₁-C₆ alkoxy, C₁-C₆ haloalkoxy, —SO₂R₁₁, —CO₂C₁-C₃ alkyl, —CONR₁₁R₁₁, —NR₁₁R₁₁, —NR₁₁CO₂R₁₁, an optionally substituted C₁-C₆ alkyl, an optionally substituted C₂-C₆ alkenyl, an optionally substituted C₂-C₆ alkynyl, an optionally substituted C₃-C₅ cycloalkyl, an optionally substituted heterocycle selected from pyrrolidine, pyrrolidinone, piperidine or morpholine, an optionally substituted phenyl, an optionally substituted 1,3-benzodioxole, an optionally substituted 2,3-dihydro-1,4-benzodioxine, or an optionally substituted heteroaryl selected from pyridine, pyrimidine, pyridazine, pyrazine, pyrazole, isoxazole, isothiazole, imidazole, oxazole, or thiazole; wherein the optionally substituted C₁-C₆ alkyl, C₂-C₆ alkenyl, or C₂-C₆ alkynyl is each optionally substituted with a —CN, —OH, oxetanyl, C₁-C₃ alkoxy, or —CONR₁₁R₁₁; the optionally substituted C₃-C₅ cycloalkyl, phenyl, 1,3-benzodioxole, 2,3-dihydro-1,4-benzodioxine, heterocycle or heteroaryl is each optionally substituted with one to three substituents each independently selected from halogen, C₁-C₃ alkyl, C₁-C₃ haloalkyl, C₁-C₃ alkoxy, C₁-C₃ haloalkoxy, —SO₂R₁₁, —NR₁₁R₁₁, —OH or —CN.
   46. The compound as defined as defined in any one of aspects 1-36, or pharmaceutically acceptable salt thereof, wherein R₂ is a cyclopropyl, cyclobutyl, cyclopentyl, or cyclohexyl, each optionally substituted with one to three R₁₀ substituents, or R₂ is a group of the formula:

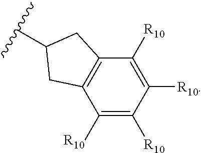

47. The compound as defined as defined in any one of aspects 1-36, or pharmaceutically acceptable salt thereof, wherein R₂ is a group of the formula:

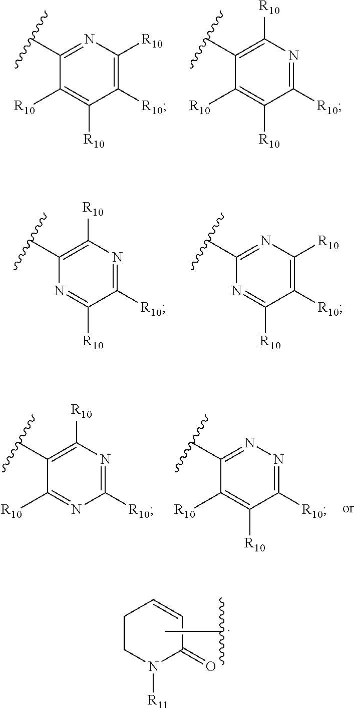

48. The compound as defined as defined in any one of aspects 1-36, or pharmaceutically acceptable salt thereof, wherein R₂ is a group of the formula:

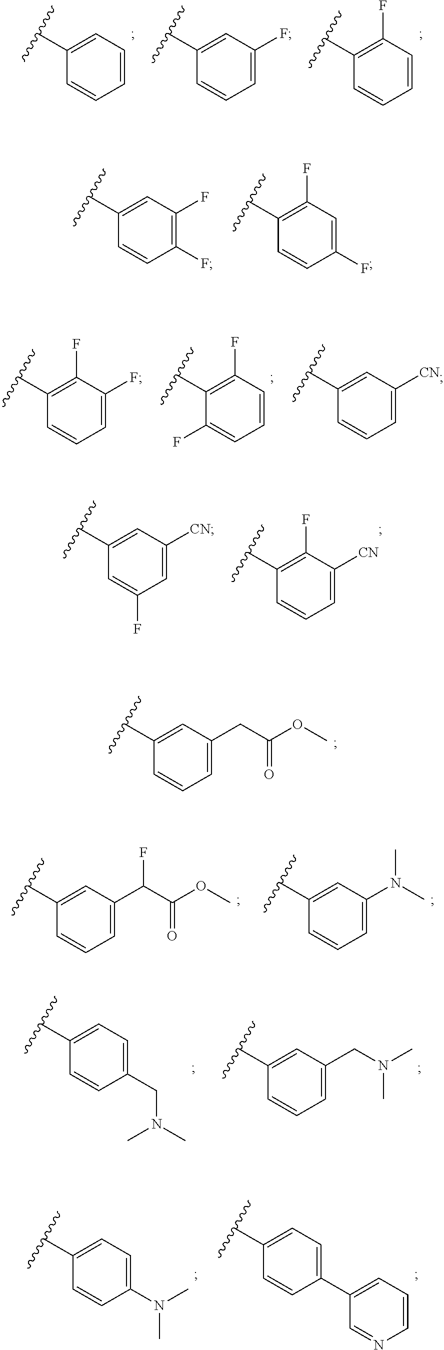

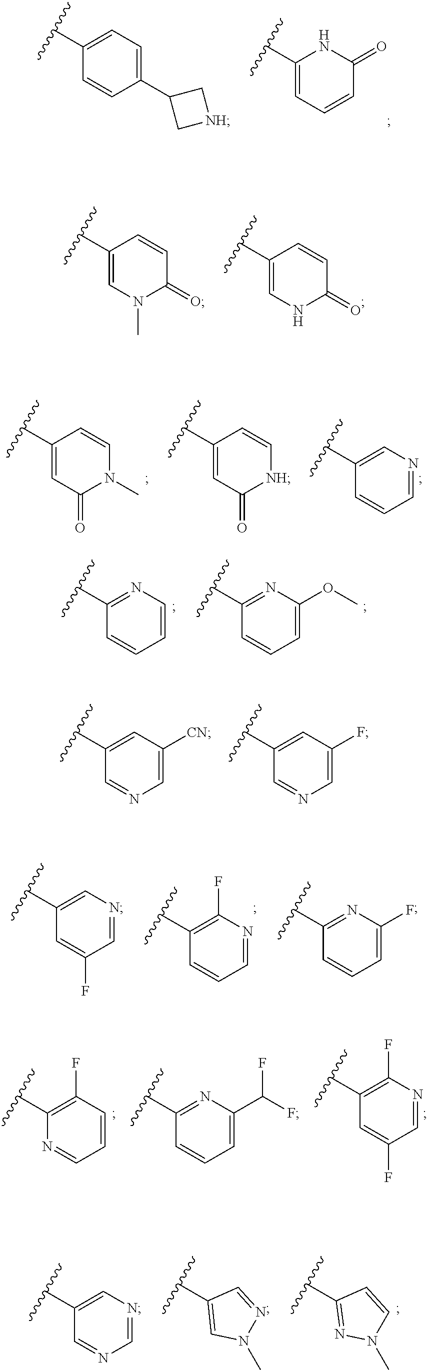

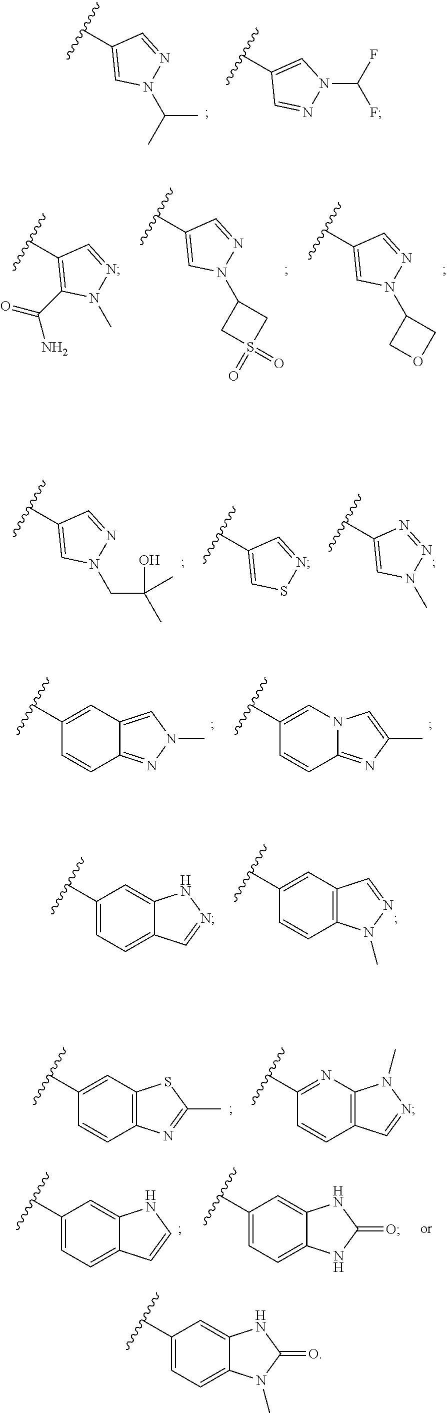

49. The compound as defined in aspect 1 or 2 selected from:

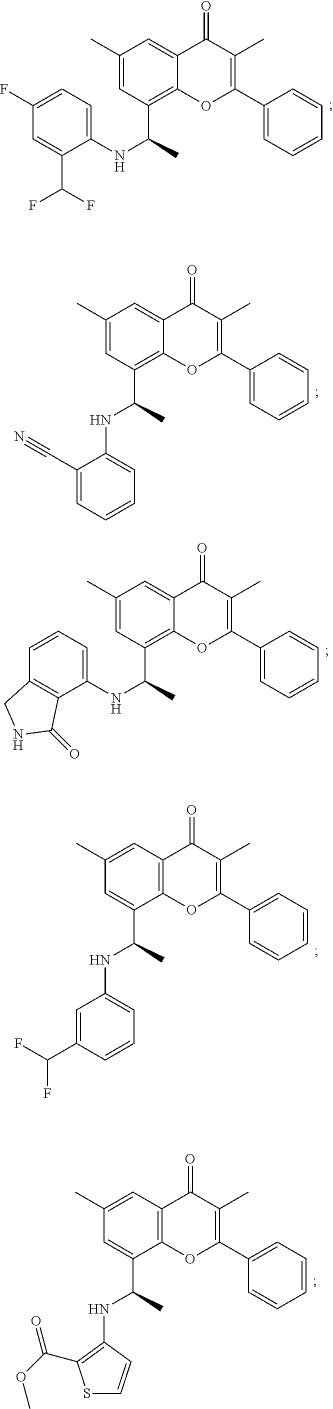 [SMILES: Cc1cc([C@@H](C)Nc2ccc(F)cc2C(F)F)c2oc(-c3ccccc3)c(C)c(=O)c2c1 ; Cc1cc([C@@H](C)Nc2ccccc2C#N)c2oc(-c3ccccc3)c(C)c(=O)c2c1 ; Cc1cc([C@@H](C)Nc2cccc3c2C(=O)NC3)c2oc(-c3ccccc3)c(C)c(=O)c2c1 ; Cc1cc([C@@H](C)Nc2cccc(C(F)F)c2)c2oc(-c3ccccc3)c(C)c(=O)c2c1 ; COC(=O)c1sccc1N[C@H](C)c1cc(C)cc2c(=O)c(C)c(-c3ccccc3)oc12]

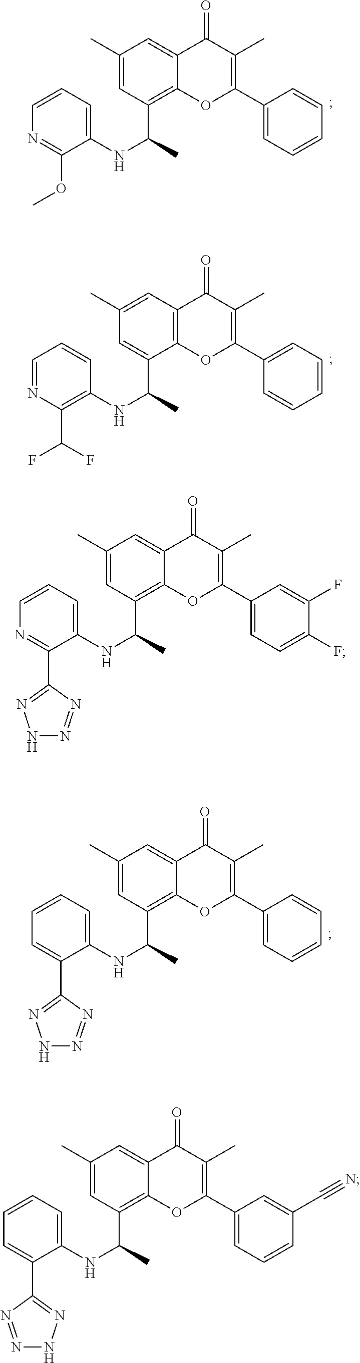 [SMILES: COc1ncccc1N[C@H](C)c1cc(C)cc2c(=O)c(C)c(-c3ccccc3)oc12 ; Cc1cc([C@@H](C)Nc2cccnc2C(F)F)c2oc(-c3ccccc3)c(C)c(=O)c2c1 ; Cc1cc([C@@H](C)Nc2cccnc2-c2nn[nH]n2)c2oc(-c3ccc(F)c(F)c3)c(C)c(=O)c2c1 ; Cc1cc([C@@H](C)Nc2ccccc2-c2nn[nH]n2)c2oc(-c3ccccc3)c(C)c(=O)c2c1 ; Cc1cc([C@@H](C)Nc2ccccc2-c2nn[nH]n2)c2oc(-c3cccc(C#N)c3)c(C)c(=O)c2c1]

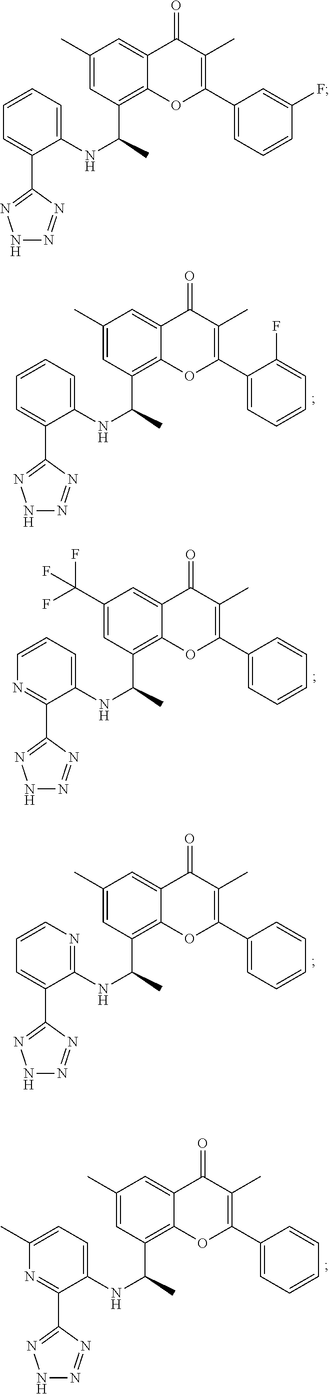

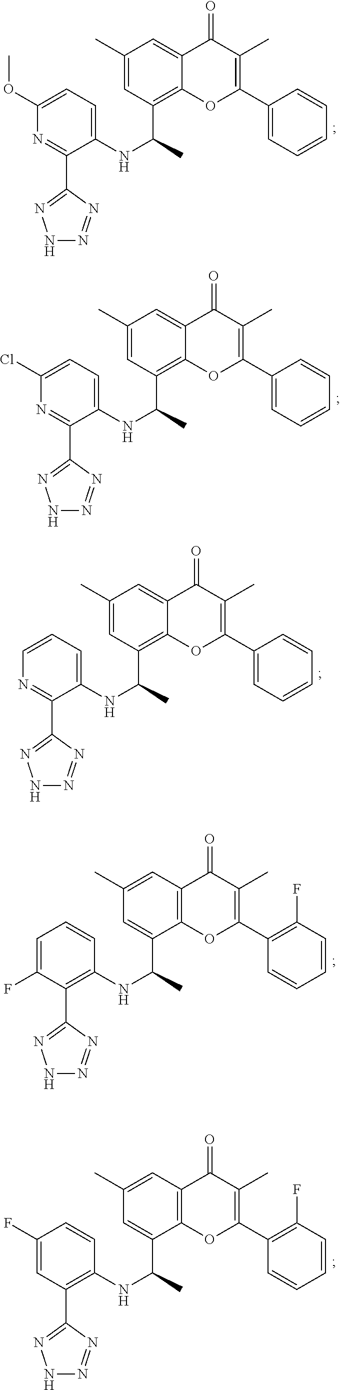 [SMILES: COc1ccc(N[C@H](C)c2cc(C)cc3c(=O)c(C)c(-c4ccccc4)oc23)c(-c2nn[nH]n2)n1 ; Cc1cc([C@@H](C)Nc2ccc(Cl)nc2-c2nn[nH]n2)c2oc(-c3ccccc3)c(C)c(=O)c2c1 ; Cc1cc([C@@H](C)Nc2cccnc2-c2nn[nH]n2)c2oc(-c3ccccc3)c(C)c(=O)c2c1 ; Cc1cc([C@@H](C)Nc2cccc(F)c2-c2nn[nH]n2)c2oc(-c3ccccc3F)c(C)c(=O)c2c1 ; Cc1cc([C@@H](C)Nc2ccc(F)cc2-c2nn[nH]n2)c2oc(-c3ccccc3F)c(C)c(=O)c2c1]

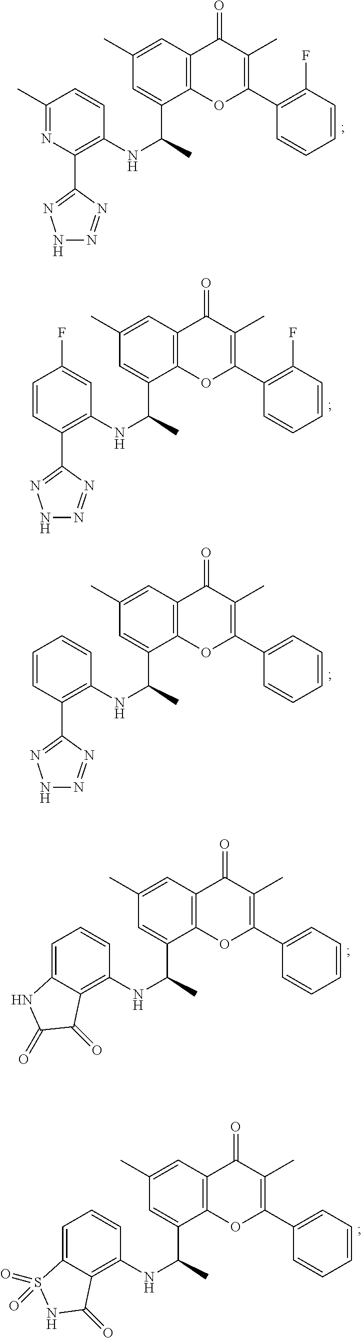 [SMILES: Cc1cc([C@@H](C)Nc2ccc(C)nc2-c2nn[nH]n2)c2oc(-c3ccccc3F)c(C)c(=O)c2c1 ; Cc1cc([C@@H](C)Nc2cc(F)ccc2-c2nn[nH]n2)c2oc(-c3ccccc3F)c(C)c(=O)c2c1 ; Cc1cc([C@@H](C)Nc2ccccc2-c2nn[nH]n2)c2oc(-c3ccccc3)c(C)c(=O)c2c1 ; Cc1cc([C@@H](C)Nc2cccc3c2C(=O)C(=O)N3)c2oc(-c3ccccc3)c(C)c(=O)c2c1 ; Cc1cc([C@@H](C)Nc2cccc3c2C(=O)NS3(=O)=O)c2oc(-c3ccccc3)c(C)c(=O)c2c1]

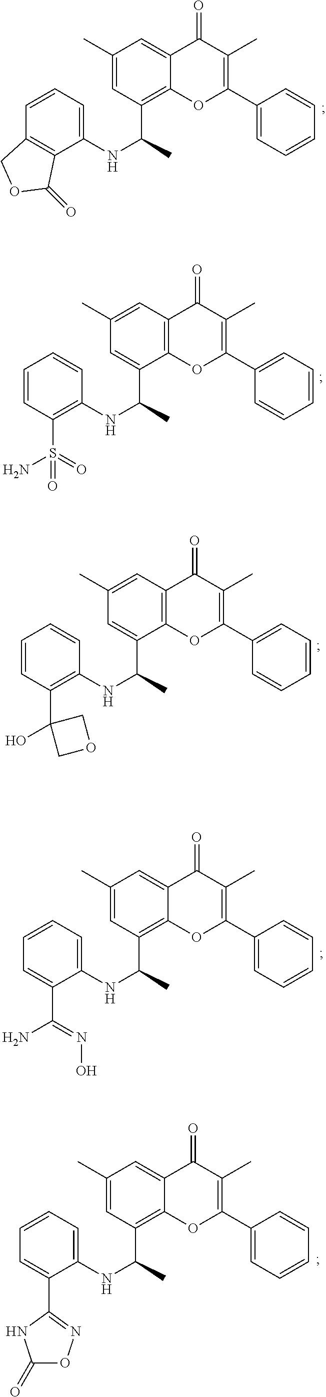 [SMILES: Cc1cc([C@@H](C)Nc2cccc3c2C(=O)OC3)c2oc(-c3ccccc3)c(C)c(=O)c2c1 ; Cc1cc([C@@H](C)Nc2ccccc2S(N)(=O)=O)c2oc(-c3ccccc3)c(C)c(=O)c2c1 ; Cc1cc([C@@H](C)Nc2ccccc2C2(O)COC2)c2oc(-c3ccccc3)c(C)c(=O)c2c1 ; Cc1cc([C@@H](C)Nc2ccccc2/C(N)=N/O)c2oc(-c3ccccc3)c(C)c(=O)c2c1 ; Cc1cc([C@@H](C)Nc2ccccc2-c2noc(=O)[nH]2)c2oc(-c3ccccc3)c(C)c(=O)c2c1]

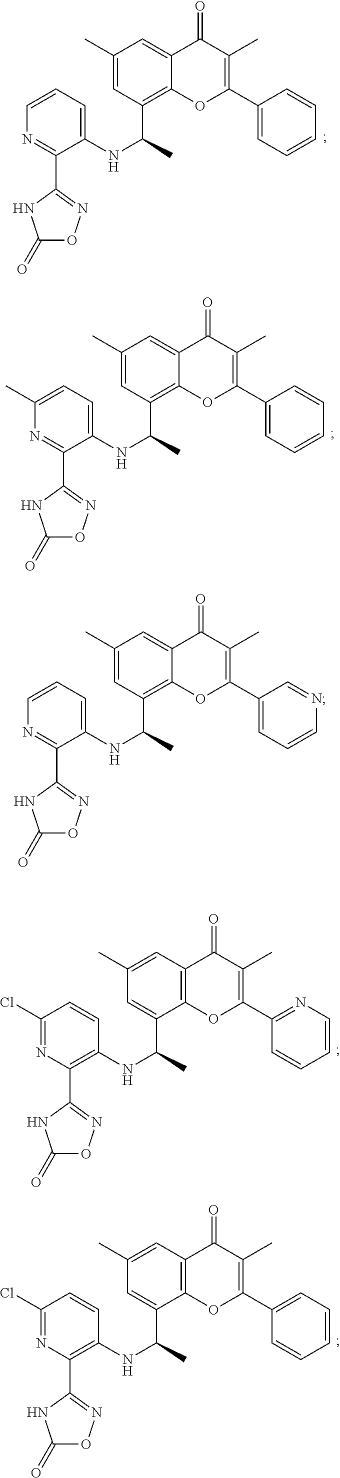 [SMILES: Cc1cc([C@@H](C)Nc2cccnc2-c2noc(=O)[nH]2)c2oc(-c3ccccc3)c(C)c(=O)c2c1 ; Cc1cc([C@@H](C)Nc2ccc(C)nc2-c2noc(=O)[nH]2)c2oc(-c3ccccc3)c(C)c(=O)c2c1 ; Cc1cc([C@@H](C)Nc2cccnc2-c2noc(=O)[nH]2)c2oc(-c3cccnc3)c(C)c(=O)c2c1 ; Cc1cc([C@@H](C)Nc2ccc(Cl)nc2-c2noc(=O)[nH]2)c2oc(-c3ccccn3)c(C)c(=O)c2c1 ; Cc1cc([C@@H](C)Nc2ccc(Cl)nc2-c2noc(=O)[nH]2)c2oc(-c3ccccc3)c(C)c(=O)c2c1]

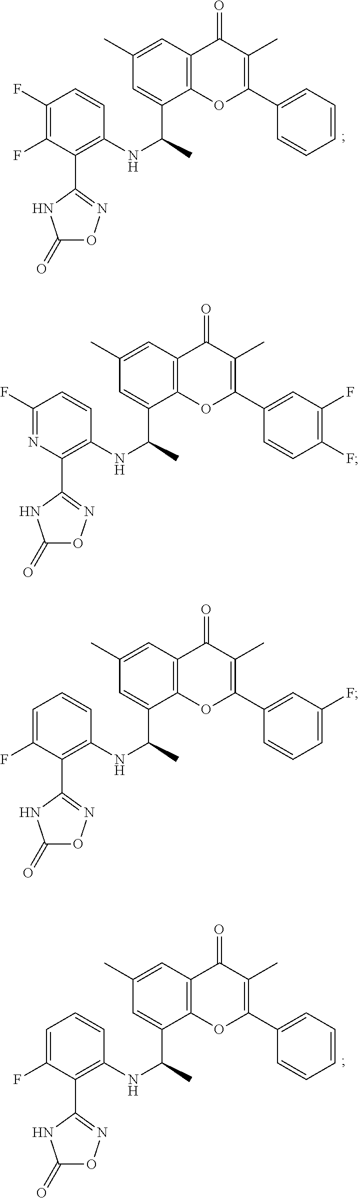 [SMILES: Cc1cc([C@@H](C)Nc2ccc(F)c(F)c2-c2noc(=O)[nH]2)c2oc(-c3ccccc3)c(C)c(=O)c2c1 ; Cc1cc([C@@H](C)Nc2ccc(F)nc2-c2noc(=O)[nH]2)c2oc(-c3ccc(F)c(F)c3)c(C)c(=O)c2c1 ; Cc1cc([C@@H](C)Nc2cccc(F)c2-c2noc(=O)[nH]2)c2oc(-c3cccc(F)c3)c(C)c(=O)c2c1 ; Cc1cc([C@@H](C)Nc2cccc(F)c2-c2noc(=O)[nH]2)c2oc(-c3ccccc3)c(C)c(=O)c2c1]

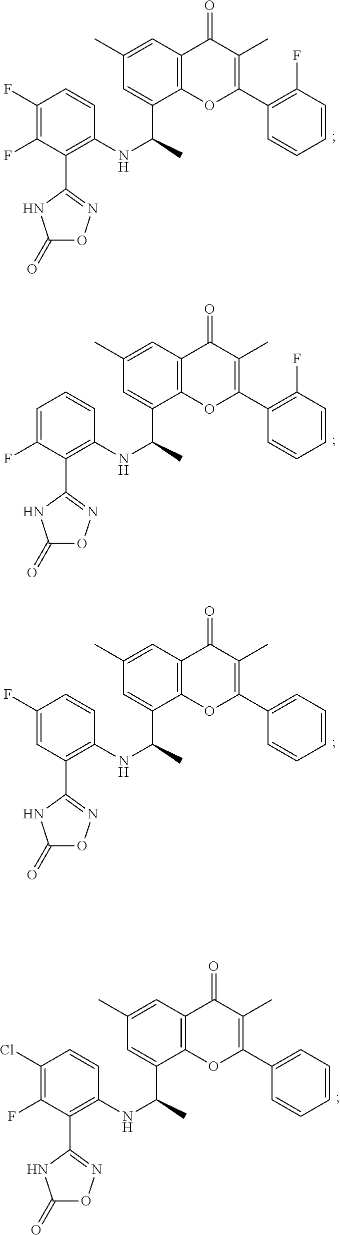 [SMILES: Cc1cc([C@@H](C)Nc2ccc(F)c(F)c2-c2noc(=O)[nH]2)c2oc(-c3ccccc3F)c(C)c(=O)c2c1 ; Cc1cc([C@@H](C)Nc2cccc(F)c2-c2noc(=O)[nH]2)c2oc(-c3ccccc3F)c(C)c(=O)c2c1 ; Cc1cc([C@@H](C)Nc2ccc(F)cc2-c2noc(=O)[nH]2)c2oc(-c3ccccc3)c(C)c(=O)c2c1 ; Cc1cc([C@@H](C)Nc2ccc(Cl)c(F)c2-c2noc(=O)[nH]2)c2oc(-c3ccccc3)c(C)c(=O)c2c1]

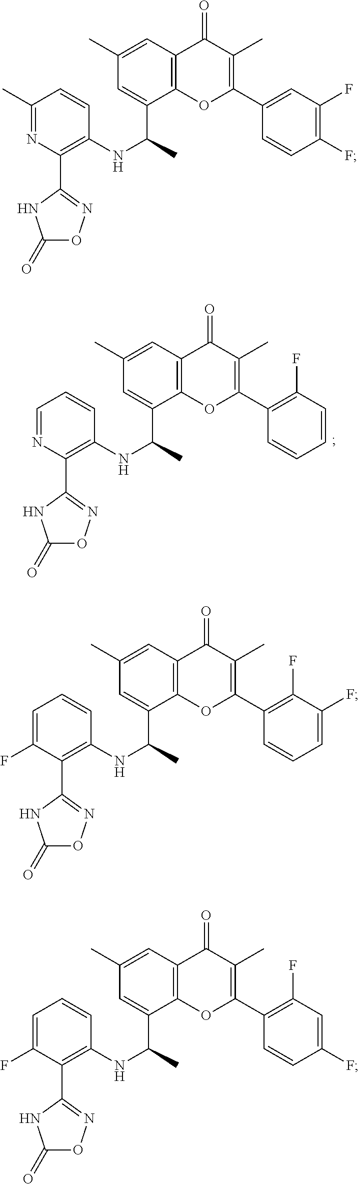 [SMILES: Cc1cc([C@@H](C)Nc2ccc(C)nc2-c2noc(=O)[nH]2)c2oc(-c3ccc(F)c(F)c3)c(C)c(=O)c2c1 ; Cc1cc([C@@H](C)Nc2cccnc2-c2noc(=O)[nH]2)c2oc(-c3ccccc3F)c(C)c(=O)c2c1 ; Cc1cc([C@@H](C)Nc2cccc(F)c2-c2noc(=O)[nH]2)c2oc(-c3cccc(F)c3F)c(C)c(=O)c2c1 ; Cc1cc([C@@H](C)Nc2cccc(F)c2-c2noc(=O)[nH]2)c2oc(-c3ccc(F)cc3F)c(C)c(=O)c2c1]

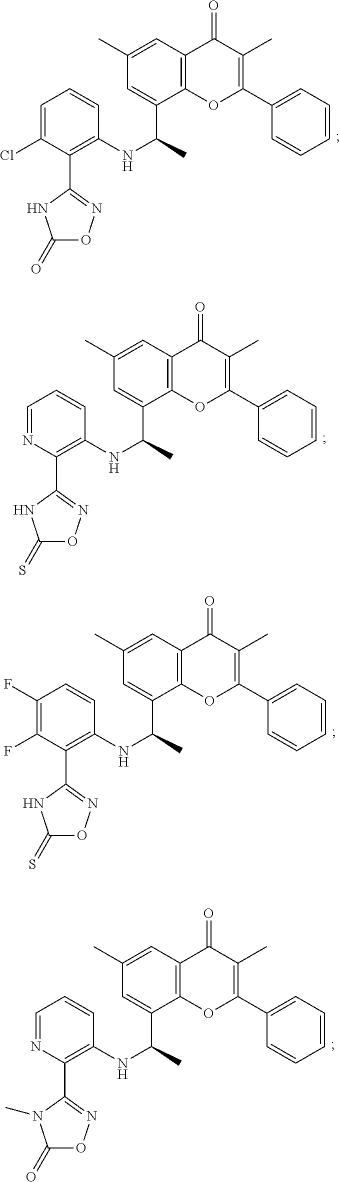 [SMILES: Cc1cc([C@@H](C)Nc2cccc(Cl)c2-c2noc(=O)[nH]2)c2oc(-c3ccccc3)c(C)c(=O)c2c1 ; Cc1cc([C@@H](C)Nc2cccnc2-c2noc(=S)[nH]2)c2oc(-c3ccccc3)c(C)c(=O)c2c1 ; Cc1cc([C@@H](C)Nc2ccc(F)c(F)c2-c2noc(=S)[nH]2)c2oc(-c3ccccc3)c(C)c(=O)c2c1 ; Cc1cc([C@@H](C)Nc2cccnc2-c2noc(=O)n2C)c2oc(-c3ccccc3)c(C)c(=O)c2c1]

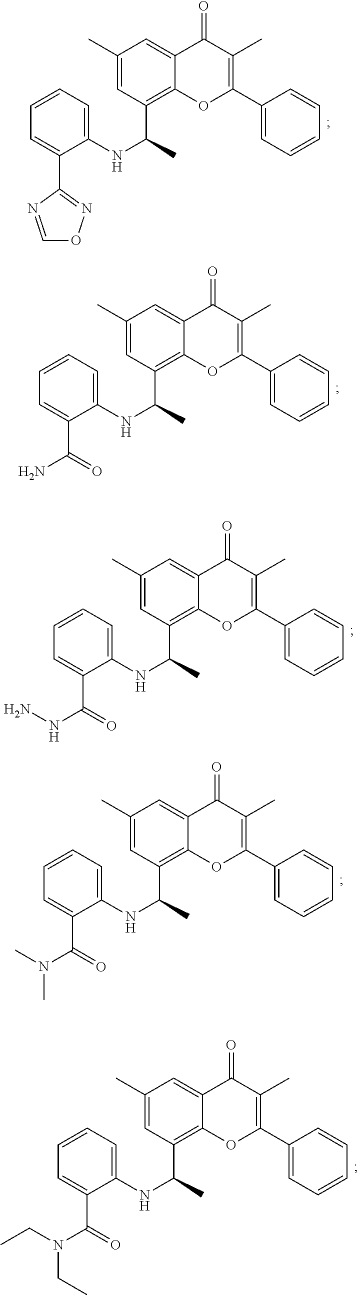 [SMILES: Cc1cc([C@@H](C)Nc2ccccc2-c2ncon2)c2oc(-c3ccccc3)c(C)c(=O)c2c1 ; Cc1cc([C@@H](C)Nc2ccccc2C(N)=O)c2oc(-c3ccccc3)c(C)c(=O)c2c1 ; Cc1cc([C@@H](C)Nc2ccccc2C(=O)NN)c2oc(-c3ccccc3)c(C)c(=O)c2c1 ; Cc1cc([C@@H](C)Nc2ccccc2C(=O)N(C)C)c2oc(-c3ccccc3)c(C)c(=O)c2c1 ; CCN(CC)C(=O)c1ccccc1N[C@H](C)c1cc(C)cc2c(=O)c(C)c(-c3ccccc3)oc12]

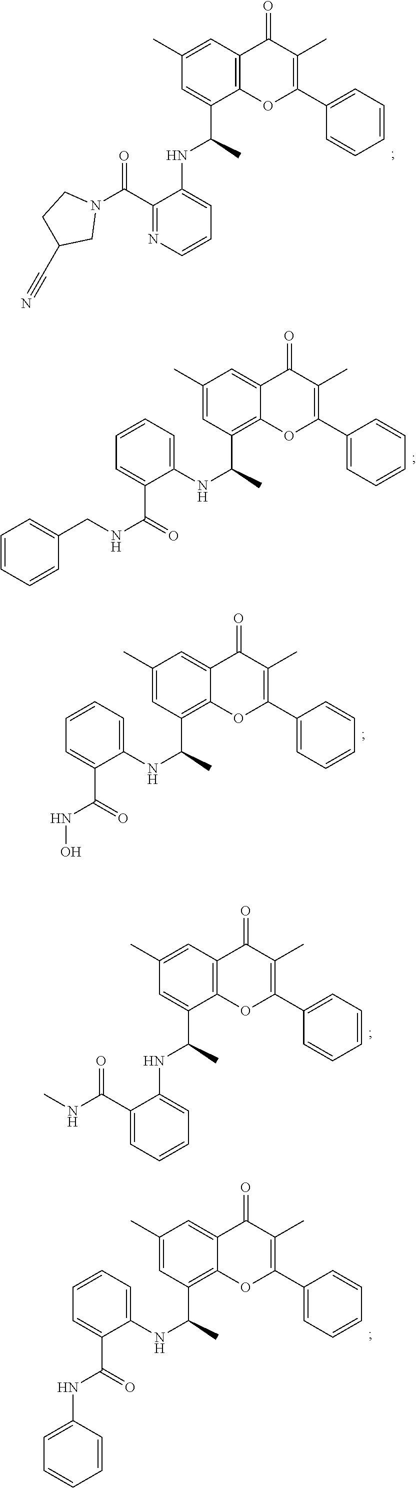 [SMILES: Cc1cc([C@@H](C)Nc2cccnc2C(=O)N2CCC(C#N)C2)c2oc(-c3ccccc3)c(C)c(=O)c2c1 ; Cc1cc([C@@H](C)Nc2ccccc2C(=O)NCc2ccccc2)c2oc(-c3ccccc3)c(C)c(=O)c2c1 ; Cc1cc([C@@H](C)Nc2ccccc2C(=O)NO)c2oc(-c3ccccc3)c(C)c(=O)c2c1 ; CNC(=O)c1ccccc1N[C@H](C)c1cc(C)cc2c(=O)c(C)c(-c3ccccc3)oc12 ; Cc1cc([C@@H](C)Nc2ccccc2C(=O)Nc2ccccc2)c2oc(-c3ccccc3)c(C)c(=O)c2c1]

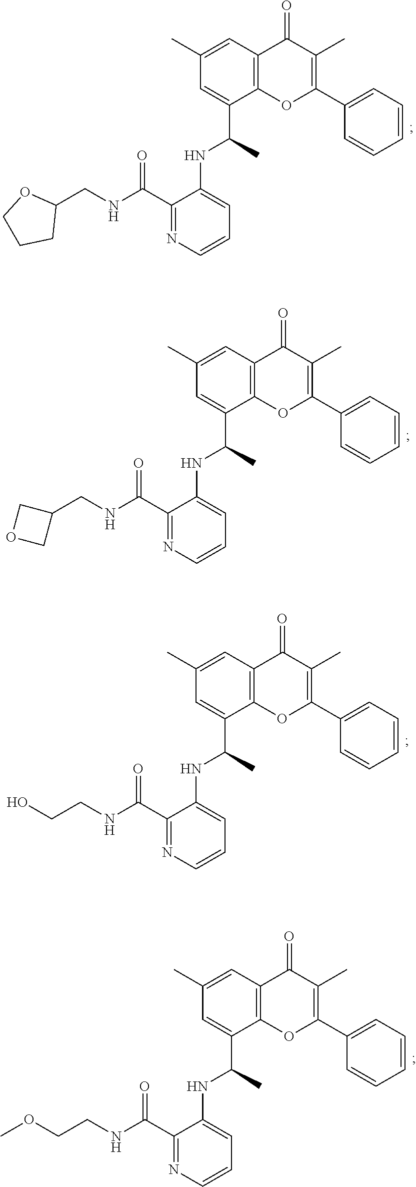 [SMILES: Cc1cc([C@@H](C)Nc2cccnc2C(=O)NCC2CCCO2)c2oc(-c3ccccc3)c(C)c(=O)c2c1 ; Cc1cc([C@@H](C)Nc2cccnc2C(=O)NCC2COC2)c2oc(-c3ccccc3)c(C)c(=O)c2c1 ; Cc1cc([C@@H](C)Nc2cccnc2C(=O)NCCO)c2oc(-c3ccccc3)c(C)c(=O)c2c1 ; COCCNC(=O)c1ncccc1N[C@H](C)c1cc(C)cc2c(=O)c(C)c(-c3ccccc3)oc12]

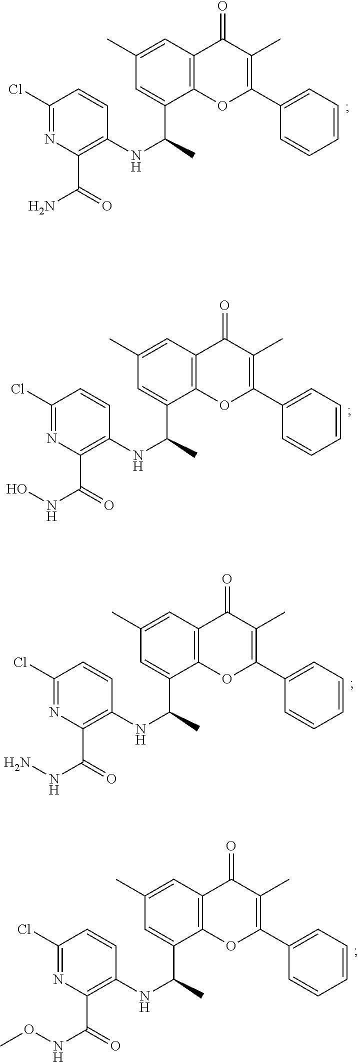 [SMILES: Cc1cc([C@@H](C)Nc2ccc(Cl)nc2C(N)=O)c2oc(-c3ccccc3)c(C)c(=O)c2c1 ; Cc1cc([C@@H](C)Nc2ccc(Cl)nc2C(=O)NO)c2oc(-c3ccccc3)c(C)c(=O)c2c1 ; Cc1cc([C@@H](C)Nc2ccc(Cl)nc2C(=O)NN)c2oc(-c3ccccc3)c(C)c(=O)c2c1 ; CONC(=O)c1nc(Cl)ccc1N[C@H](C)c1cc(C)cc2c(=O)c(C)c(-c3ccccc3)oc12]

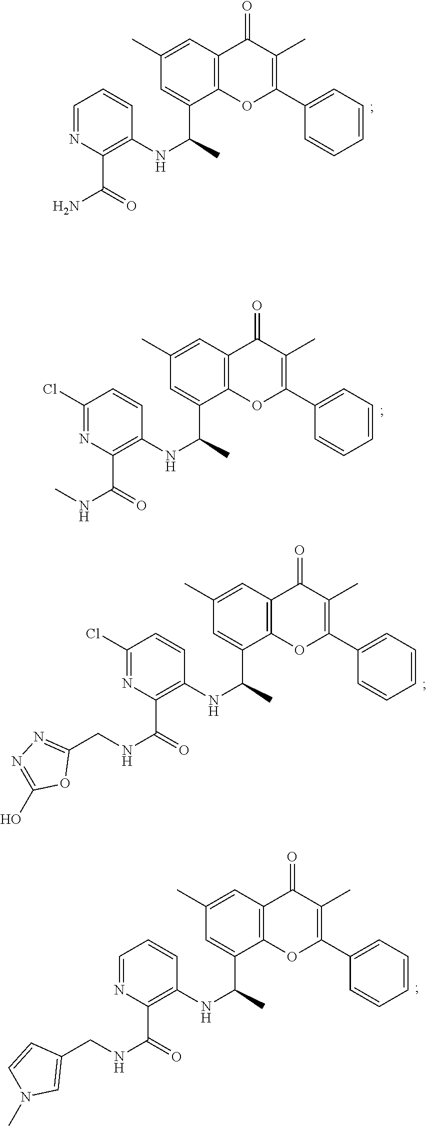 [SMILES: Cc1cc([C@@H](C)Nc2cccnc2C(N)=O)c2oc(-c3ccccc3)c(C)c(=O)c2c1 ; CNC(=O)c1nc(Cl)ccc1N[C@H](C)c1cc(C)cc2c(=O)c(C)c(-c3ccccc3)oc12 ; Cc1cc([C@@H](C)Nc2ccc(Cl)nc2C(=O)NCc2nnc(O)o2)c2oc(-c3ccccc3)c(C)c(=O)c2c1 ; Cc1cc([C@@H](C)Nc2cccnc2C(=O)NCc2ccn(C)c2)c2oc(-c3ccccc3)c(C)c(=O)c2c1]

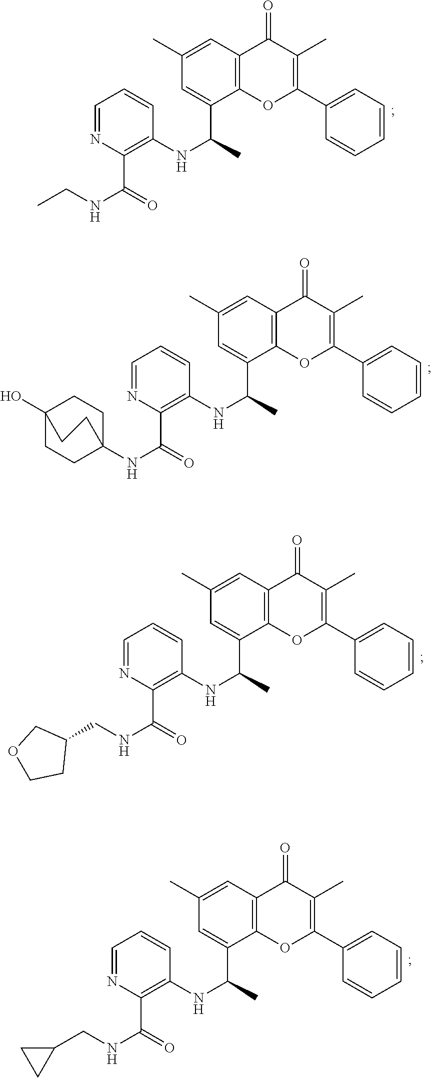 [SMILES: CCNC(=O)c1ncccc1N[C@H](C)c1cc(C)cc2c(=O)c(C)c(-c3ccccc3)oc12 ; Cc1cc([C@@H](C)Nc2cccnc2C(=O)NC23CCC(O)(CC2)CC3)c2oc(-c3ccccc3)c(C)c(=O)c2c1 ; Cc1cc([C@@H](C)Nc2cccnc2C(=O)NC[C@@H]2CCOC2)c2oc(-c3ccccc3)c(C)c(=O)c2c1 ; Cc1cc([C@@H](C)Nc2cccnc2C(=O)NCC2CC2)c2oc(-c3ccccc3)c(C)c(=O)c2c1]

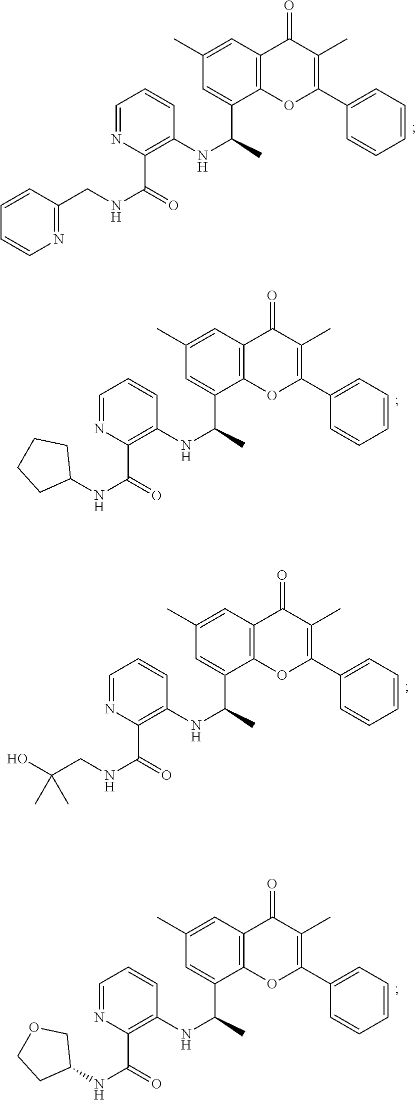 [SMILES: Cc1cc([C@@H](C)Nc2cccnc2C(=O)NCc2ccccn2)c2oc(-c3ccccc3)c(C)c(=O)c2c1 ; Cc1cc([C@@H](C)Nc2cccnc2C(=O)NC2CCCC2)c2oc(-c3ccccc3)c(C)c(=O)c2c1 ; Cc1cc([C@@H](C)Nc2cccnc2C(=O)NCC(C)(C)O)c2oc(-c3ccccc3)c(C)c(=O)c2c1 ; Cc1cc([C@@H](C)Nc2cccnc2C(=O)N[C@@H]2CCOC2)c2oc(-c3ccccc3)c(C)c(=O)c2c1]

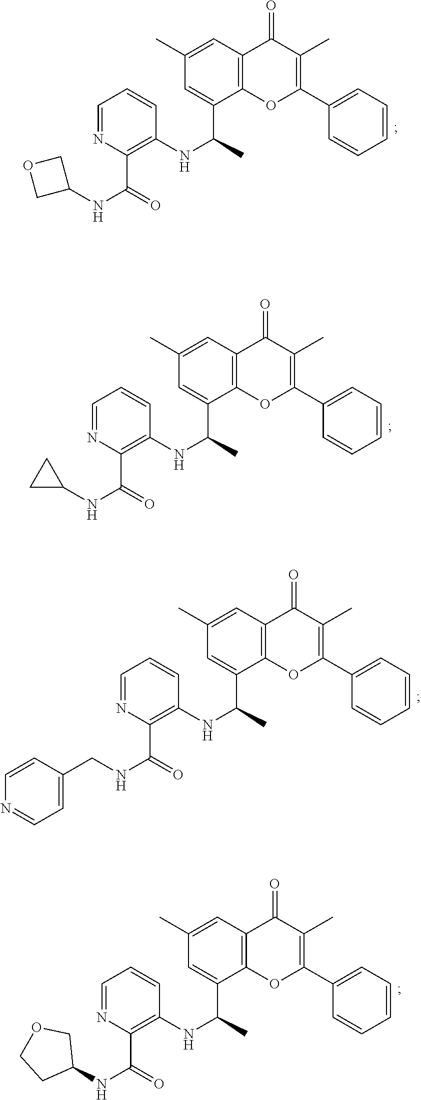 [SMILES: Cc1cc([C@@H](C)Nc2cccnc2C(=O)NC2COC2)c2oc(-c3ccccc3)c(C)c(=O)c2c1 ; Cc1cc([C@@H](C)Nc2cccnc2C(=O)NC2CC2)c2oc(-c3ccccc3)c(C)c(=O)c2c1 ; Cc1cc([C@@H](C)Nc2cccnc2C(=O)NCc2ccncc2)c2oc(-c3ccccc3)c(C)c(=O)c2c1 ; Cc1cc([C@@H](C)Nc2cccnc2C(=O)N[C@H]2CCOC2)c2oc(-c3ccccc3)c(C)c(=O)c2c1]

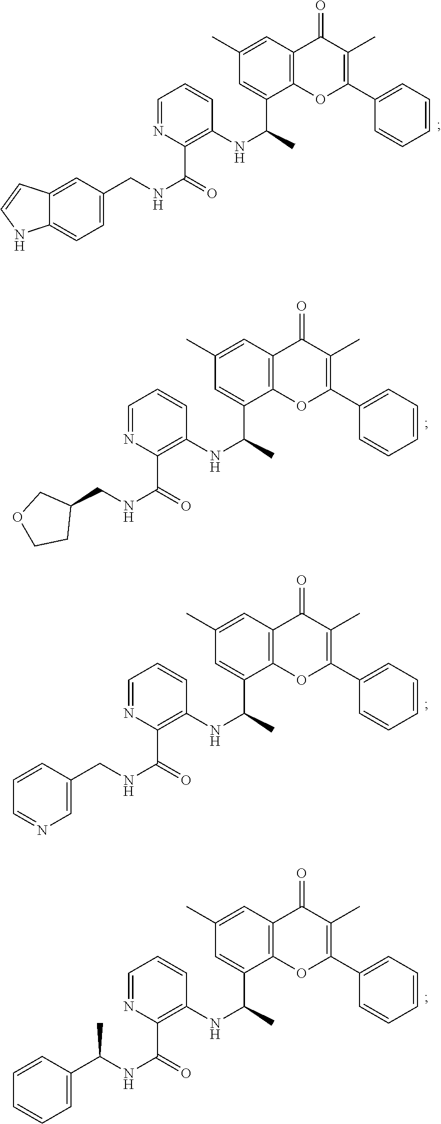 [SMILES: Cc1cc([C@@H](C)Nc2cccnc2C(=O)NCc2ccc3[nH]ccc3c2)c2oc(-c3ccccc3)c(C)c(=O)c2c1 ; Cc1cc([C@@H](C)Nc2cccnc2C(=O)NC[C@H]2CCOC2)c2oc(-c3ccccc3)c(C)c(=O)c2c1 ; Cc1cc([C@@H](C)Nc2cccnc2C(=O)NCc2cccnc2)c2oc(-c3ccccc3)c(C)c(=O)c2c1 ; Cc1cc([C@@H](C)Nc2cccnc2C(=O)N[C@H](C)c2ccccc2)c2oc(-c3ccccc3)c(C)c(=O)c2c1]

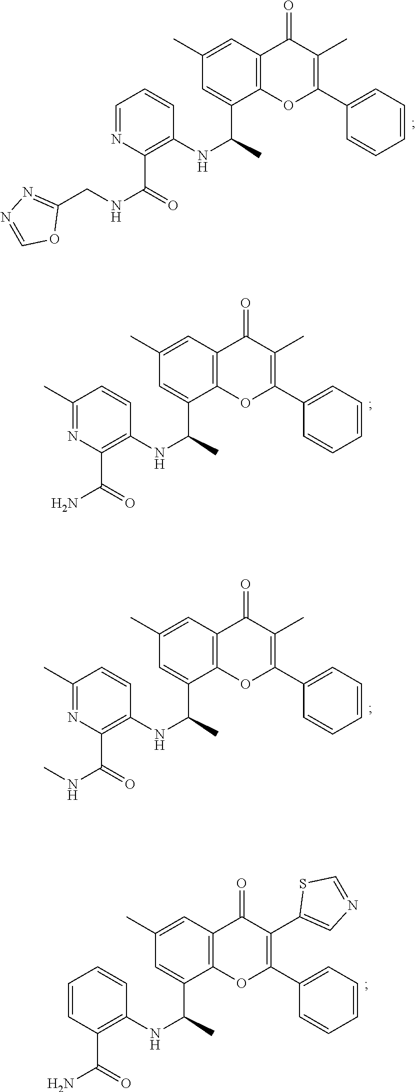 [SMILES: Cc1cc([C@@H](C)Nc2cccnc2C(=O)NCc2nnco2)c2oc(-c3ccccc3)c(C)c(=O)c2c1 ; Cc1cc([C@@H](C)Nc2ccc(C)nc2C(N)=O)c2oc(-c3ccccc3)c(C)c(=O)c2c1 ; CNC(=O)c1nc(C)ccc1N[C@H](C)c1cc(C)cc2c(=O)c(C)c(-c3ccccc3)oc12 ; Cc1cc([C@@H](C)Nc2ccccc2C(N)=O)c2oc(-c3ccccc3)c(-c3cncs3)c(=O)c2c1]

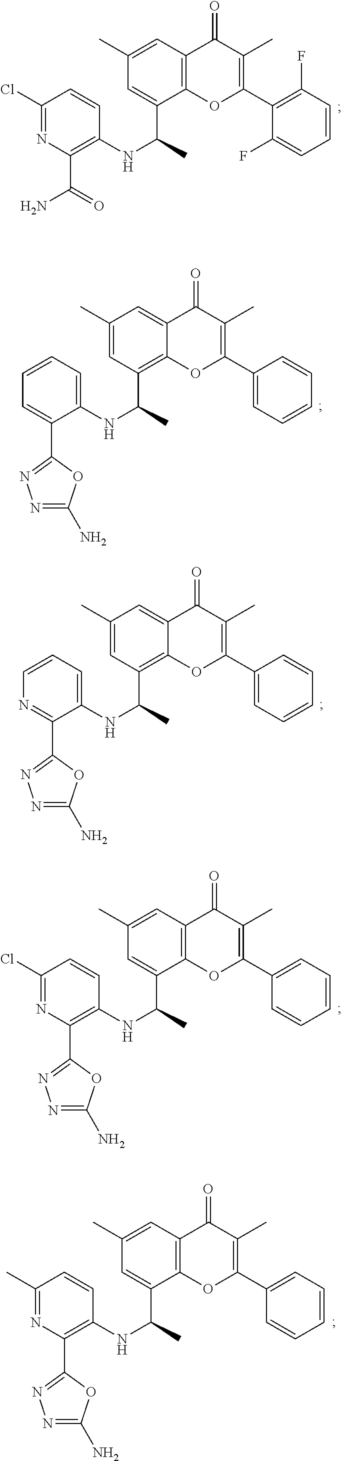 [SMILES: Cc1cc([C@@H](C)Nc2ccc(Cl)nc2C(N)=O)c2oc(-c3c(F)cccc3F)c(C)c(=O)c2c1 ; Cc1cc([C@@H](C)Nc2ccccc2-c2nnc(N)o2)c2oc(-c3ccccc3)c(C)c(=O)c2c1 ; Cc1cc([C@@H](C)Nc2cccnc2-c2nnc(N)o2)c2oc(-c3ccccc3)c(C)c(=O)c2c1 ; Cc1cc([C@@H](C)Nc2ccc(Cl)nc2-c2nnc(N)o2)c2oc(-c3ccccc3)c(C)c(=O)c2c1 ; Cc1cc([C@@H](C)Nc2ccc(C)nc2-c2nnc(N)o2)c2oc(-c3ccccc3)c(C)c(=O)c2c1]

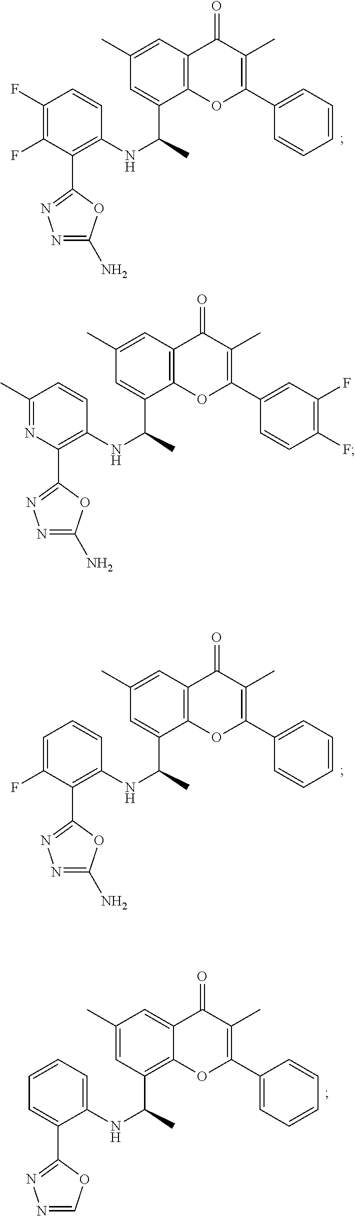 [SMILES: Cc1cc([C@@H](C)Nc2ccc(F)c(F)c2-c2nnc(N)o2)c2oc(-c3ccccc3)c(C)c(=O)c2c1 ; Cc1cc([C@@H](C)Nc2ccc(C)nc2-c2nnc(N)o2)c2oc(-c3ccc(F)c(F)c3)c(C)c(=O)c2c1 ; Cc1cc([C@@H](C)Nc2cccc(F)c2-c2nnc(N)o2)c2oc(-c3ccccc3)c(C)c(=O)c2c1 ; Cc1cc([C@@H](C)Nc2ccccc2-c2nnco2)c2oc(-c3ccccc3)c(C)c(=O)c2c1]

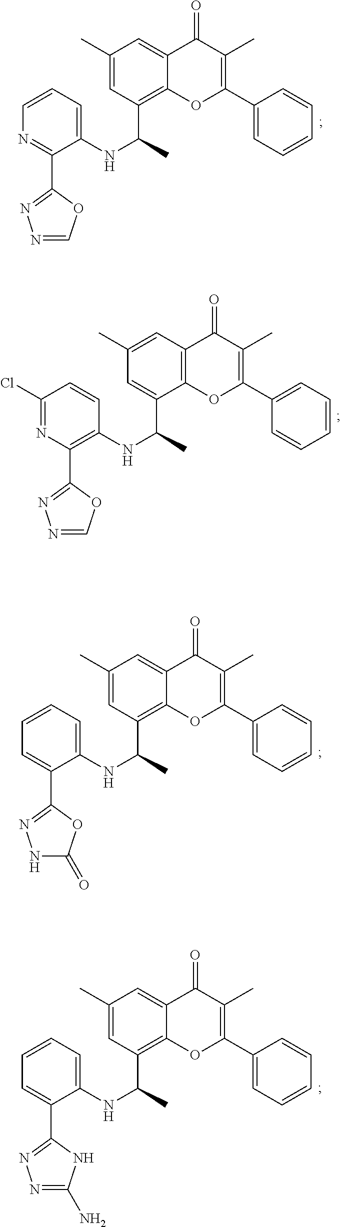 [SMILES: Cc1cc([C@@H](C)Nc2cccnc2-c2nnco2)c2oc(-c3ccccc3)c(C)c(=O)c2c1 ; Cc1cc([C@@H](C)Nc2ccc(Cl)nc2-c2nnco2)c2oc(-c3ccccc3)c(C)c(=O)c2c1 ; Cc1cc([C@@H](C)Nc2ccccc2-c2n[nH]c(=O)o2)c2oc(-c3ccccc3)c(C)c(=O)c2c1 ; Cc1cc([C@@H](C)Nc2ccccc2-c2nnc(N)[nH]2)c2oc(-c3ccccc3)c(C)c(=O)c2c1]

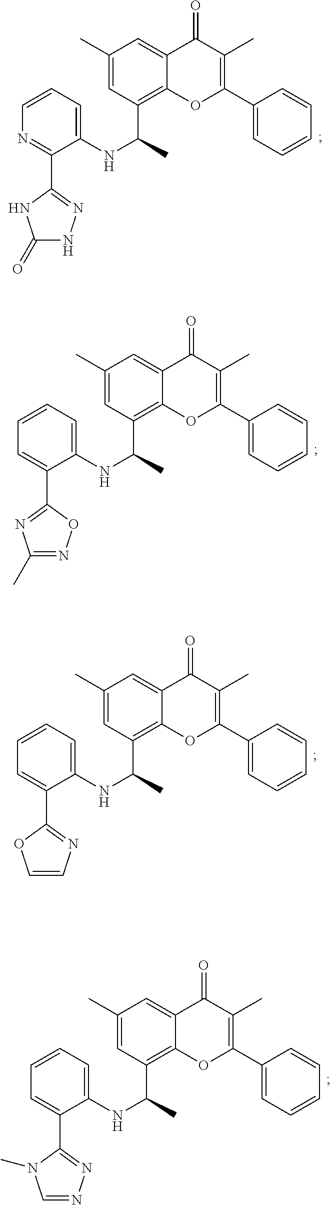 [SMILES: Cc1cc([C@@H](C)Nc2cccnc2-c2n[nH]c(=O)[nH]2)c2oc(-c3ccccc3)c(C)c(=O)c2c1 ; Cc1cc([C@@H](C)Nc2ccccc2-c2nc(C)no2)c2oc(-c3ccccc3)c(C)c(=O)c2c1 ; Cc1cc([C@@H](C)Nc2ccccc2-c2ncco2)c2oc(-c3ccccc3)c(C)c(=O)c2c1 ; Cc1cc([C@@H](C)Nc2ccccc2-c2nncn2C)c2oc(-c3ccccc3)c(C)c(=O)c2c1]

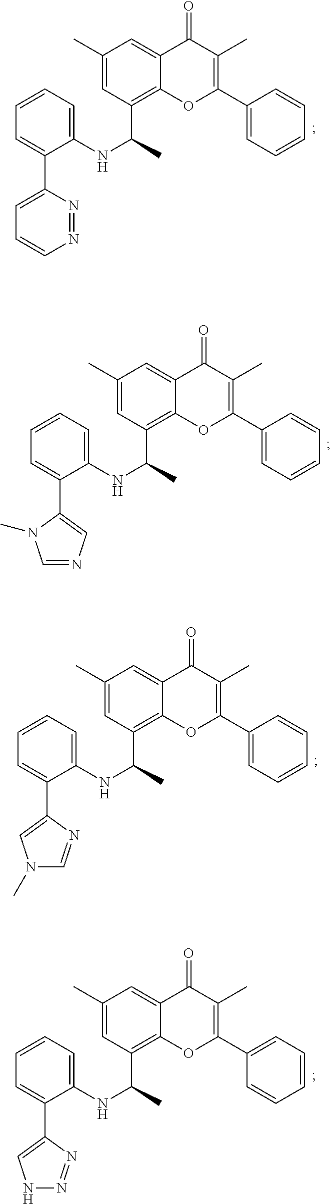 [SMILES: Cc1cc([C@@H](C)Nc2ccccc2-c2cccnn2)c2oc(-c3ccccc3)c(C)c(=O)c2c1 ; Cc1cc([C@@H](C)Nc2ccccc2-c2cncn2C)c2oc(-c3ccccc3)c(C)c(=O)c2c1 ; Cc1cc([C@@H](C)Nc2ccccc2-c2cn(C)cn2)c2oc(-c3ccccc3)c(C)c(=O)c2c1 ; Cc1cc([C@@H](C)Nc2ccccc2-c2c[nH]nn2)c2oc(-c3ccccc3)c(C)c(=O)c2c1]

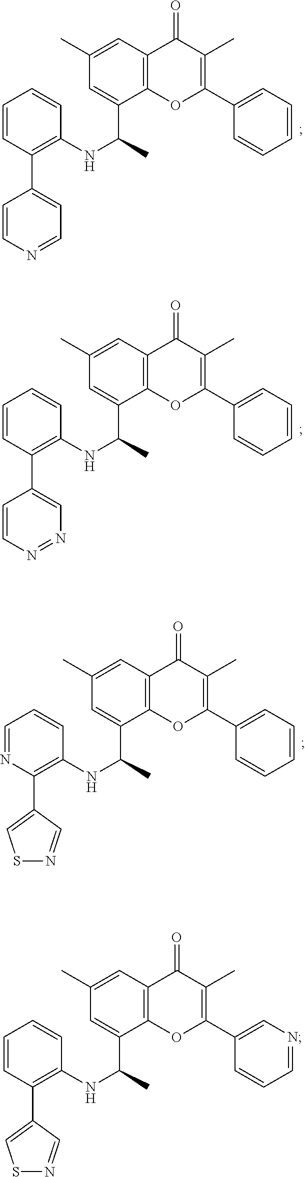 [SMILES: Cc1cc([C@@H](C)Nc2ccccc2-c2ccncc2)c2oc(-c3ccccc3)c(C)c(=O)c2c1 ; Cc1cc([C@@H](C)Nc2ccccc2-c2ccnnc2)c2oc(-c3ccccc3)c(C)c(=O)c2c1 ; Cc1cc([C@@H](C)Nc2cccnc2-c2cnsc2)c2oc(-c3ccccc3)c(C)c(=O)c2c1 ; Cc1cc([C@@H](C)Nc2ccccc2-c2cnsc2)c2oc(-c3cccnc3)c(C)c(=O)c2c1]

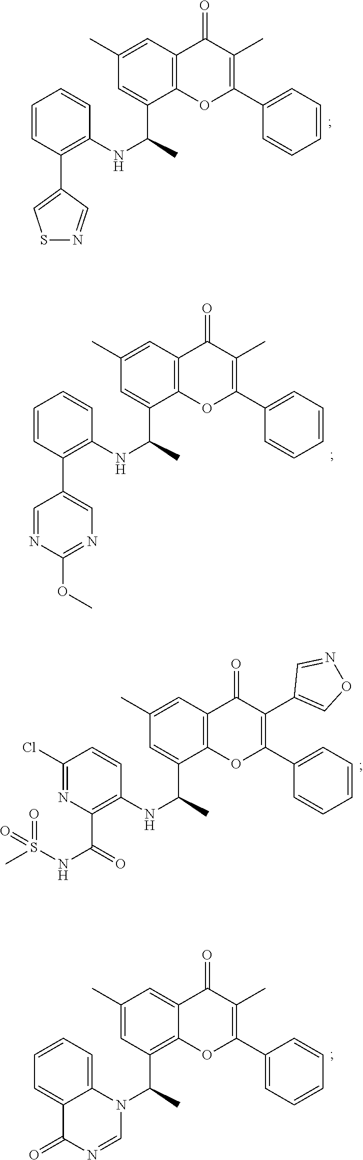 [SMILES: Cc1cc([C@@H](C)Nc2ccccc2-c2cnsc2)c2oc(-c3ccccc3)c(C)c(=O)c2c1 ; COc1ncc(-c2ccccc2N[C@H](C)c2cc(C)cc3c(=O)c(C)c(-c4ccccc4)oc23)cn1 ; Cc1cc([C@@H](C)Nc2ccc(Cl)nc2C(=O)NS(C)(=O)=O)c2oc(-c3ccccc3)c(-c3cnoc3)c(=O)c2c1 ; Cc1cc([C@@H](C)n2cnc(=O)c3ccccc32)c2oc(-c3ccccc3)c(C)c(=O)c2c1]

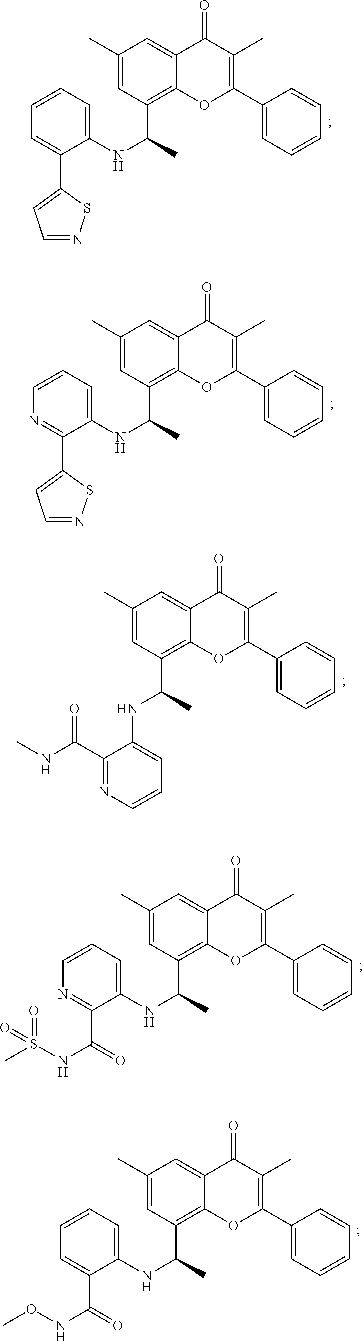 [SMILES: Cc1cc([C@@H](C)Nc2ccccc2-c2ccns2)c2oc(-c3ccccc3)c(C)c(=O)c2c1 ; Cc1cc([C@@H](C)Nc2cccnc2-c2ccns2)c2oc(-c3ccccc3)c(C)c(=O)c2c1 ; CNC(=O)c1ncccc1N[C@H](C)c1cc(C)cc2c(=O)c(C)c(-c3ccccc3)oc12 ; Cc1cc([C@@H](C)Nc2cccnc2C(=O)NS(C)(=O)=O)c2oc(-c3ccccc3)c(C)c(=O)c2c1 ; CONC(=O)c1ccccc1N[C@H](C)c1cc(C)cc2c(=O)c(C)c(-c3ccccc3)oc12]

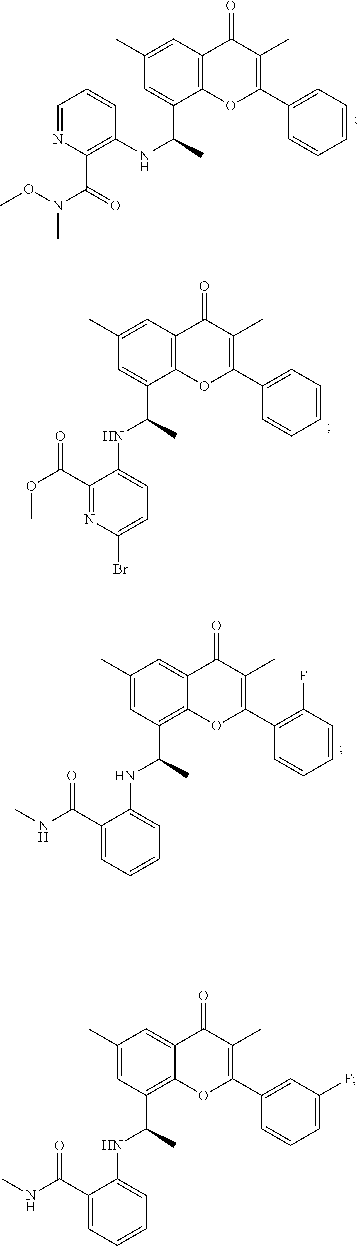 [SMILES: CON(C)C(=O)c1ncccc1N[C@H](C)c1cc(C)cc2c(=O)c(C)c(-c3ccccc3)oc12 ; COC(=O)c1nc(Br)ccc1N[C@H](C)c1cc(C)cc2c(=O)c(C)c(-c3ccccc3)oc12 ; CNC(=O)c1ccccc1N[C@H](C)c1cc(C)cc2c(=O)c(C)c(-c3ccccc3F)oc12 ; CNC(=O)c1ccccc1N[C@H](C)c1cc(C)cc2c(=O)c(C)c(-c3cccc(F)c3)oc12]

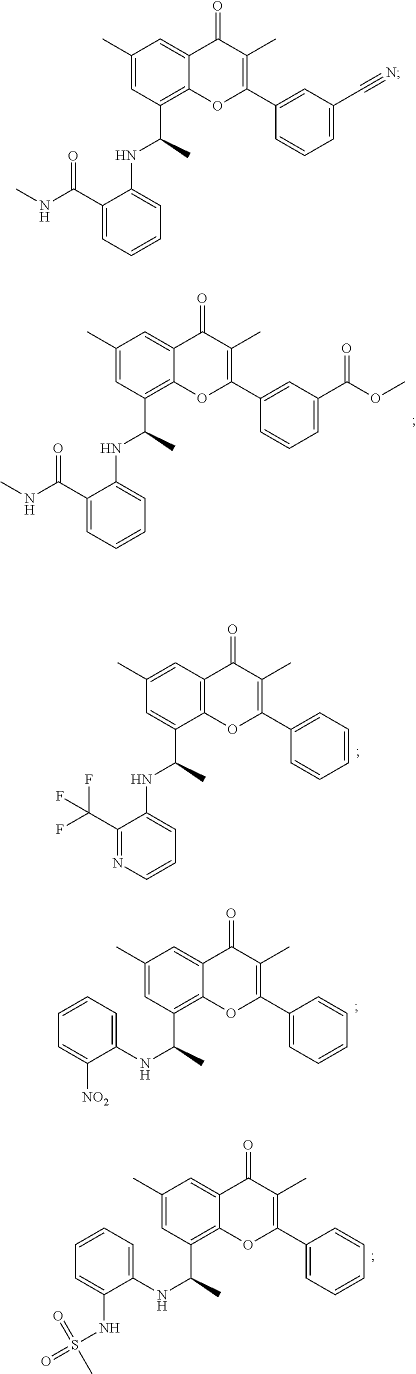

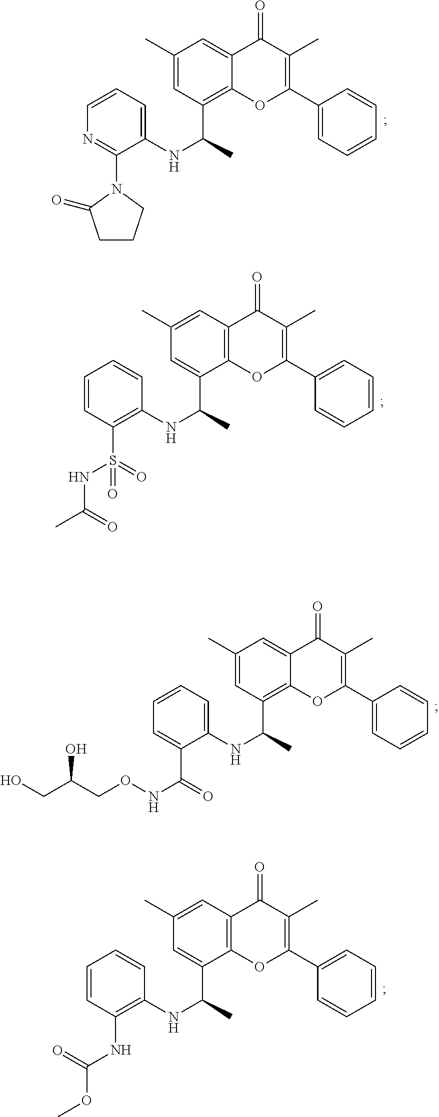 [SMILES: Cc1cc([C@@H](C)Nc2cccnc2N2CCCC2=O)c2oc(-c3ccccc3)c(C)c(=O)c2c1 ; CC(=O)NS(=O)(=O)c1ccccc1N[C@H](C)c1cc(C)cc2c(=O)c(C)c(-c3ccccc3)oc12 ; Cc1cc([C@@H](C)Nc2ccccc2C(=O)NOC[C@H](O)CO)c2oc(-c3ccccc3)c(C)c(=O)c2c1 ; COC(=O)Nc1ccccc1N[C@H](C)c1cc(C)cc2c(=O)c(C)c(-c3ccccc3)oc12]

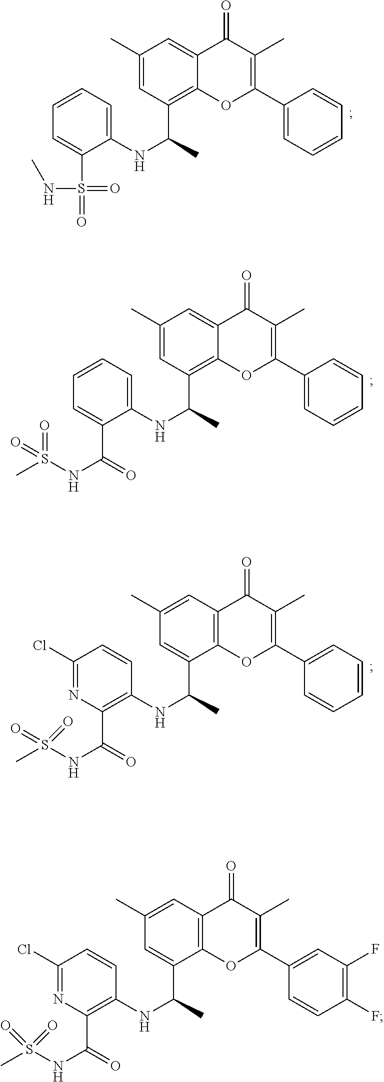 [SMILES: CNS(=O)(=O)c1ccccc1N[C@H](C)c1cc(C)cc2c(=O)c(C)c(-c3ccccc3)oc12 ; Cc1cc([C@@H](C)Nc2ccccc2C(=O)NS(C)(=O)=O)c2oc(-c3ccccc3)c(C)c(=O)c2c1 ; Cc1cc([C@@H](C)Nc2ccc(Cl)nc2C(=O)NS(C)(=O)=O)c2oc(-c3ccccc3)c(C)c(=O)c2c1 ; Cc1cc([C@@H](C)Nc2ccc(Cl)nc2C(=O)NS(C)(=O)=O)c2oc(-c3ccc(F)c(F)c3)c(C)c(=O)c2c1]

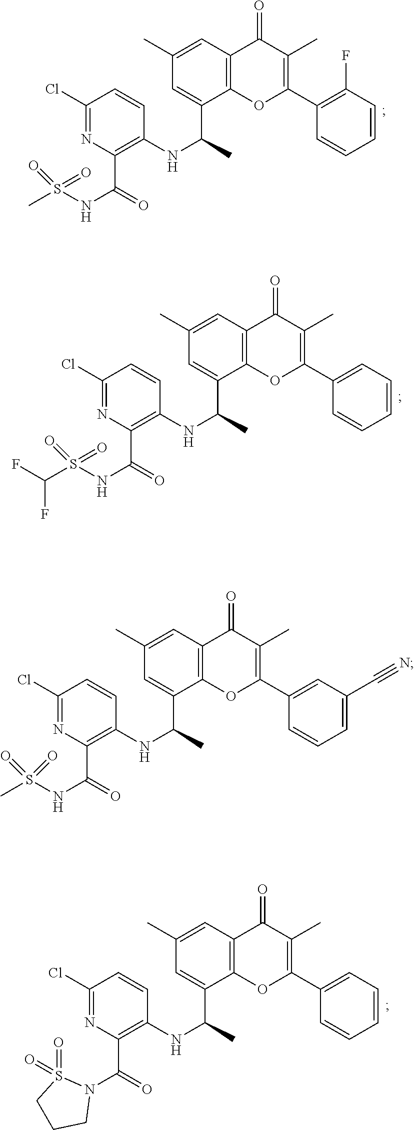 [SMILES: Cc1cc([C@@H](C)Nc2ccc(Cl)nc2C(=O)NS(C)(=O)=O)c2oc(-c3ccccc3F)c(C)c(=O)c2c1 ; Cc1cc([C@@H](C)Nc2ccc(Cl)nc2C(=O)NS(=O)(=O)C(F)F)c2oc(-c3ccccc3)c(C)c(=O)c2c1 ; Cc1cc([C@@H](C)Nc2ccc(Cl)nc2C(=O)NS(C)(=O)=O)c2oc(-c3cccc(C#N)c3)c(C)c(=O)c2c1 ; Cc1cc([C@@H](C)Nc2ccc(Cl)nc2C(=O)N2CCCS2(=O)=O)c2oc(-c3ccccc3)c(C)c(=O)c2c1]

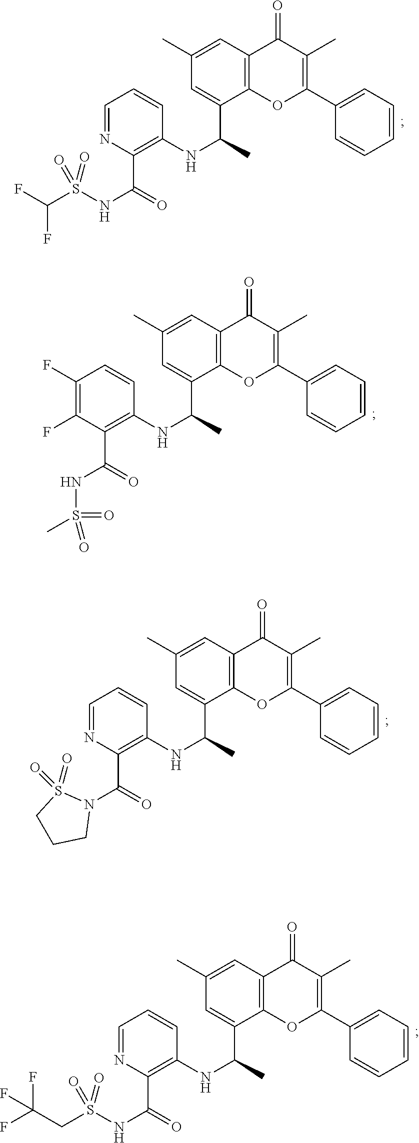 [SMILES: Cc1cc([C@@H](C)Nc2cccnc2C(=O)NS(=O)(=O)C(F)F)c2oc(-c3ccccc3)c(C)c(=O)c2c1 ; Cc1cc([C@@H](C)Nc2ccc(F)c(F)c2C(=O)NS(C)(=O)=O)c2oc(-c3ccccc3)c(C)c(=O)c2c1 ; Cc1cc([C@@H](C)Nc2cccnc2C(=O)N2CCCS2(=O)=O)c2oc(-c3ccccc3)c(C)c(=O)c2c1 ; Cc1cc([C@@H](C)Nc2cccnc2C(=O)NS(=O)(=O)CC(F)(F)F)c2oc(-c3ccccc3)c(C)c(=O)c2c1]

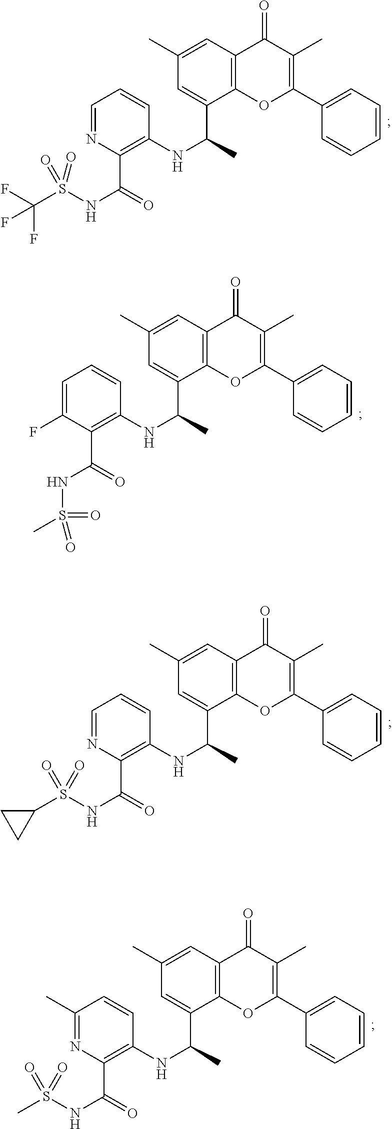 [SMILES: Cc1cc([C@@H](C)Nc2cccnc2C(=O)NS(=O)(=O)C(F)(F)F)c2oc(-c3ccccc3)c(C)c(=O)c2c1 ; Cc1cc([C@@H](C)Nc2cccc(F)c2C(=O)NS(C)(=O)=O)c2oc(-c3ccccc3)c(C)c(=O)c2c1 ; Cc1cc([C@@H](C)Nc2cccnc2C(=O)NS(=O)(=O)C2CC2)c2oc(-c3ccccc3)c(C)c(=O)c2c1 ; Cc1cc([C@@H](C)Nc2ccc(C)nc2C(=O)NS(C)(=O)=O)c2oc(-c3ccccc3)c(C)c(=O)c2c1]

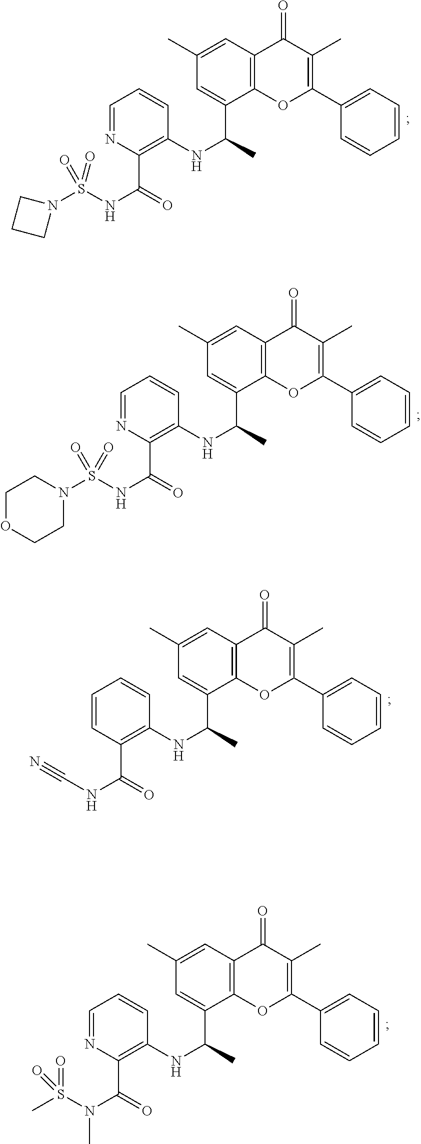 [SMILES: Cc1cc([C@@H](C)Nc2cccnc2C(=O)NS(=O)(=O)N2CCC2)c2oc(-c3ccccc3)c(C)c(=O)c2c1 ; Cc1cc([C@@H](C)Nc2cccnc2C(=O)NS(=O)(=O)N2CCOCC2)c2oc(-c3ccccc3)c(C)c(=O)c2c1 ; Cc1cc([C@@H](C)Nc2ccccc2C(=O)NC#N)c2oc(-c3ccccc3)c(C)c(=O)c2c1 ; Cc1cc([C@@H](C)Nc2cccnc2C(=O)N(C)S(C)(=O)=O)c2oc(-c3ccccc3)c(C)c(=O)c2c1]

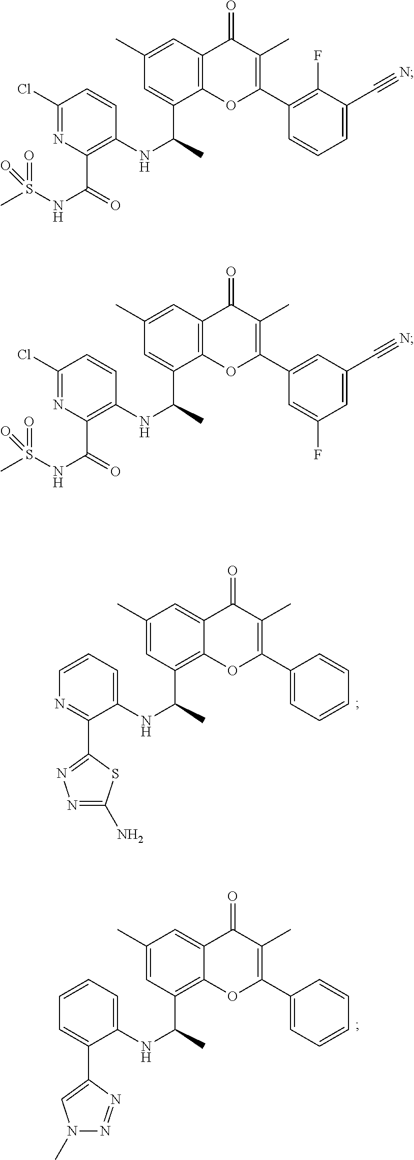 [SMILES: Cc1cc([C@@H](C)Nc2ccc(Cl)nc2C(=O)NS(C)(=O)=O)c2oc(-c3cccc(C#N)c3F)c(C)c(=O)c2c1 ; Cc1cc([C@@H](C)Nc2ccc(Cl)nc2C(=O)NS(C)(=O)=O)c2oc(-c3cc(F)cc(C#N)c3)c(C)c(=O)c2c1 ; Cc1cc([C@@H](C)Nc2cccnc2-c2nnc(N)s2)c2oc(-c3ccccc3)c(C)c(=O)c2c1 ; Cc1cc([C@@H](C)Nc2ccccc2-c2cn(C)nn2)c2oc(-c3ccccc3)c(C)c(=O)c2c1]

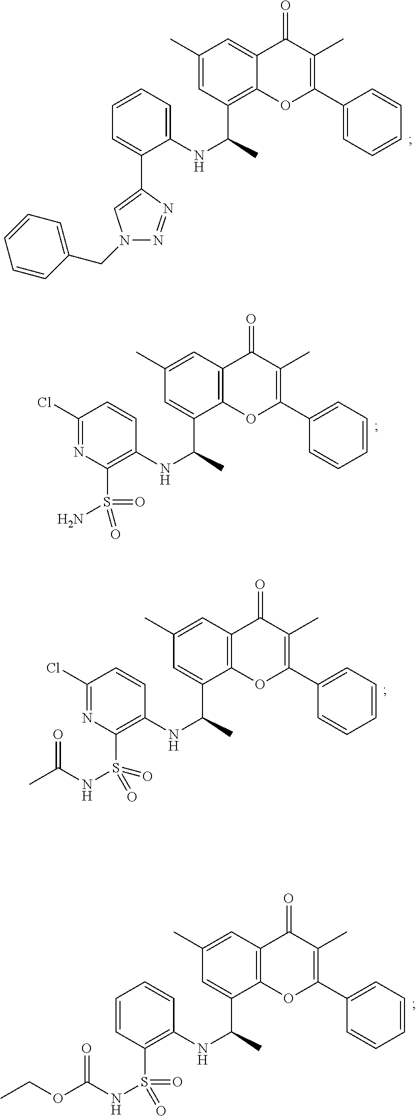 [SMILES: Cc1cc([C@@H](C)Nc2ccccc2-c2cn(Cc3ccccc3)nn2)c2oc(-c3ccccc3)c(C)c(=O)c2c1 ; Cc1cc([C@@H](C)Nc2ccc(Cl)nc2S(N)(=O)=O)c2oc(-c3ccccc3)c(C)c(=O)c2c1 ; CC(=O)NS(=O)(=O)c1nc(Cl)ccc1N[C@H](C)c1cc(C)cc2c(=O)c(C)c(-c3ccccc3)oc12 ; CCOC(=O)NS(=O)(=O)c1ccccc1N[C@H](C)c1cc(C)cc2c(=O)c(C)c(-c3ccccc3)oc12]

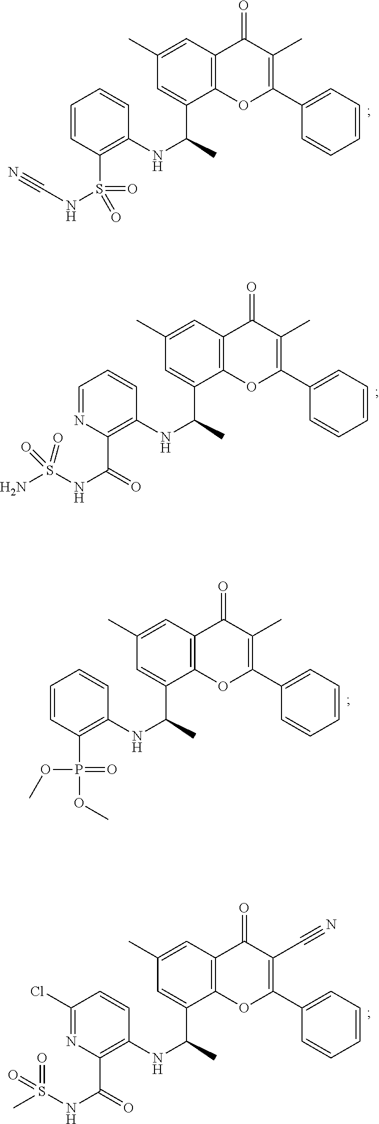 [SMILES: Cc1cc([C@@H](C)Nc2ccccc2S(=O)(=O)NC#N)c2oc(-c3ccccc3)c(C)c(=O)c2c1 ; Cc1cc([C@@H](C)Nc2cccnc2C(=O)NS(N)(=O)=O)c2oc(-c3ccccc3)c(C)c(=O)c2c1 ; COP(=O)(OC)c1ccccc1N[C@H](C)c1cc(C)cc2c(=O)c(C)c(-c3ccccc3)oc12 ; Cc1cc([C@@H](C)Nc2ccc(Cl)nc2C(=O)NS(C)(=O)=O)c2oc(-c3ccccc3)c(C#N)c(=O)c2c1]

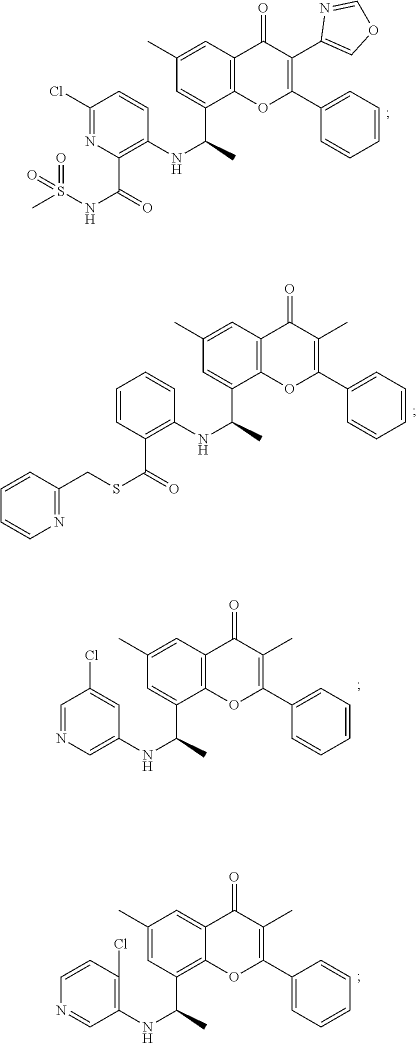 [SMILES: Cc1cc([C@@H](C)Nc2ccc(Cl)nc2C(=O)NS(C)(=O)=O)c2oc(-c3ccccc3)c(-c3cocn3)c(=O)c2c1 ; Cc1cc([C@@H](C)Nc2ccccc2C(=O)SCc2ccccn2)c2oc(-c3ccccc3)c(C)c(=O)c2c1 ; Cc1cc([C@@H](C)Nc2cncc(Cl)c2)c2oc(-c3ccccc3)c(C)c(=O)c2c1 ; Cc1cc([C@@H](C)Nc2cnccc2Cl)c2oc(-c3ccccc3)c(C)c(=O)c2c1]

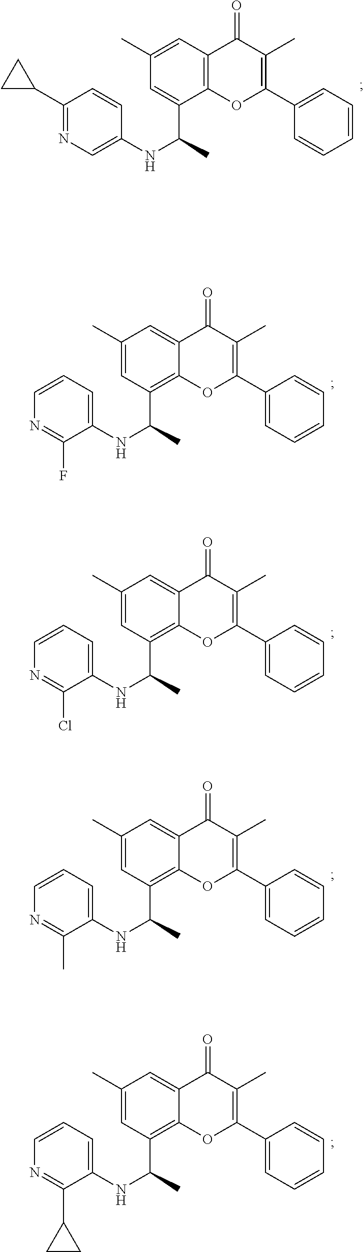 [SMILES: Cc1cc([C@@H](C)Nc2ccc(C3CC3)nc2)c2oc(-c3ccccc3)c(C)c(=O)c2c1 ; Cc1cc([C@@H](C)Nc2cccnc2F)c2oc(-c3ccccc3)c(C)c(=O)c2c1 ; Cc1cc([C@@H](C)Nc2cccnc2Cl)c2oc(-c3ccccc3)c(C)c(=O)c2c1 ; Cc1cc([C@@H](C)Nc2cccnc2C)c2oc(-c3ccccc3)c(C)c(=O)c2c1 ; Cc1cc([C@@H](C)Nc2cccnc2C2CC2)c2oc(-c3ccccc3)c(C)c(=O)c2c1]

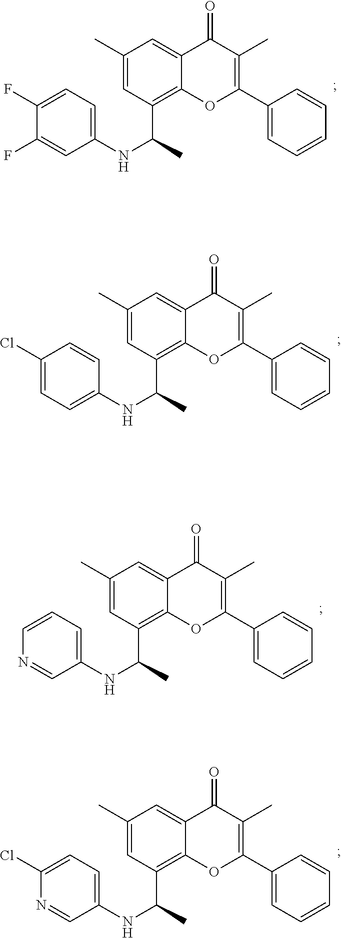 [SMILES: Cc1cc([C@@H](C)Nc2ccc(F)c(F)c2)c2oc(-c3ccccc3)c(C)c(=O)c2c1 ; Cc1cc([C@@H](C)Nc2ccc(Cl)cc2)c2oc(-c3ccccc3)c(C)c(=O)c2c1 ; Cc1cc([C@@H](C)Nc2cccnc2)c2oc(-c3ccccc3)c(C)c(=O)c2c1 ; Cc1cc([C@@H](C)Nc2ccc(Cl)nc2)c2oc(-c3ccccc3)c(C)c(=O)c2c1]

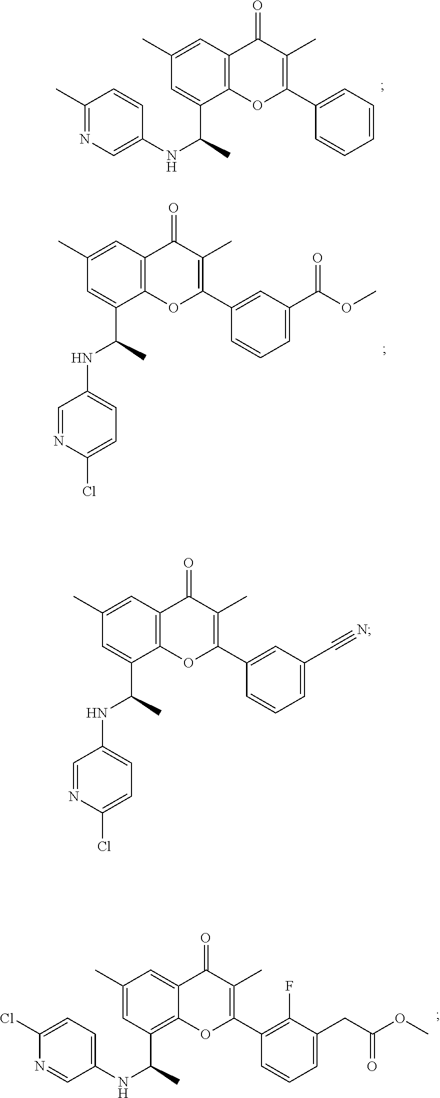 [SMILES: Cc1cc([C@@H](C)Nc2ccc(C)nc2)c2oc(-c3ccccc3)c(C)c(=O)c2c1 ; COC(=O)c1cccc(-c2oc3c([C@@H](C)Nc4ccc(Cl)nc4)cc(C)cc3c(=O)c2C)c1 ; Cc1cc([C@@H](C)Nc2ccc(Cl)nc2)c2oc(-c3cccc(C#N)c3)c(C)c(=O)c2c1 ; COC(=O)Cc1cccc(-c2oc3c([C@@H](C)Nc4ccc(Cl)nc4)cc(C)cc3c(=O)c2C)c1F]

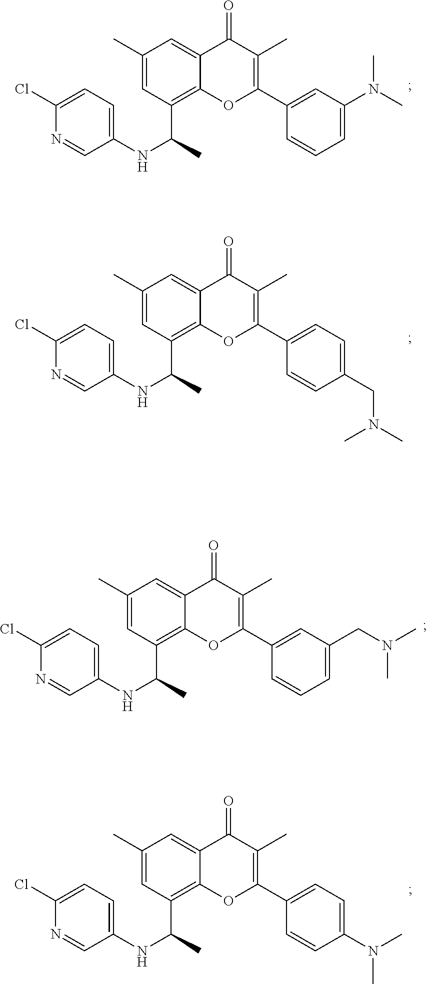 [SMILES: Cc1cc([C@@H](C)Nc2ccc(Cl)nc2)c2oc(-c3cccc(N(C)C)c3)c(C)c(=O)c2c1 ; Cc1cc([C@@H](C)Nc2ccc(Cl)nc2)c2oc(-c3ccc(CN(C)C)cc3)c(C)c(=O)c2c1 ; Cc1cc([C@@H](C)Nc2ccc(Cl)nc2)c2oc(-c3cccc(CN(C)C)c3)c(C)c(=O)c2c1 ; Cc1cc([C@@H](C)Nc2ccc(Cl)nc2)c2oc(-c3ccc(N(C)C)cc3)c(C)c(=O)c2c1]

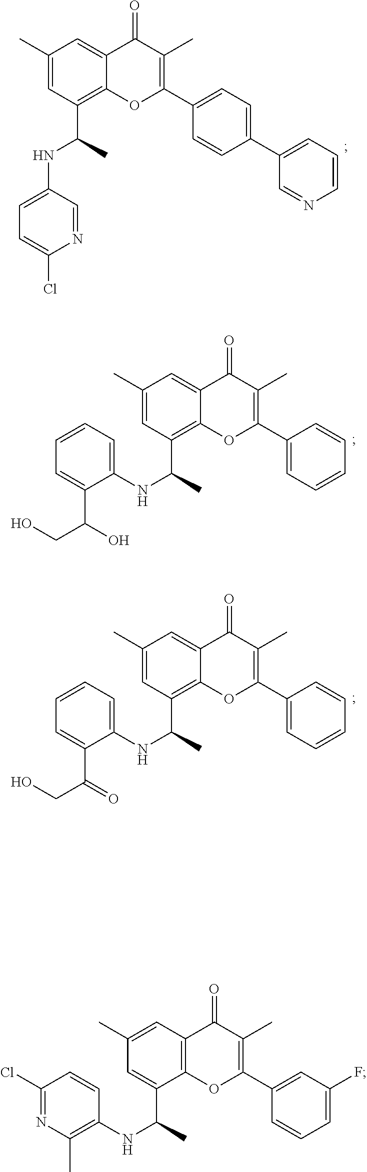 [SMILES: Cc1cc([C@@H](C)Nc2ccc(Cl)nc2)c2oc(-c3ccc(-c4cccnc4)cc3)c(C)c(=O)c2c1 ; Cc1cc([C@@H](C)Nc2ccccc2C(O)CO)c2oc(-c3ccccc3)c(C)c(=O)c2c1 ; Cc1cc([C@@H](C)Nc2ccccc2C(=O)CO)c2oc(-c3ccccc3)c(C)c(=O)c2c1 ; Cc1cc([C@@H](C)Nc2ccc(Cl)nc2C)c2oc(-c3cccc(F)c3)c(C)c(=O)c2c1]

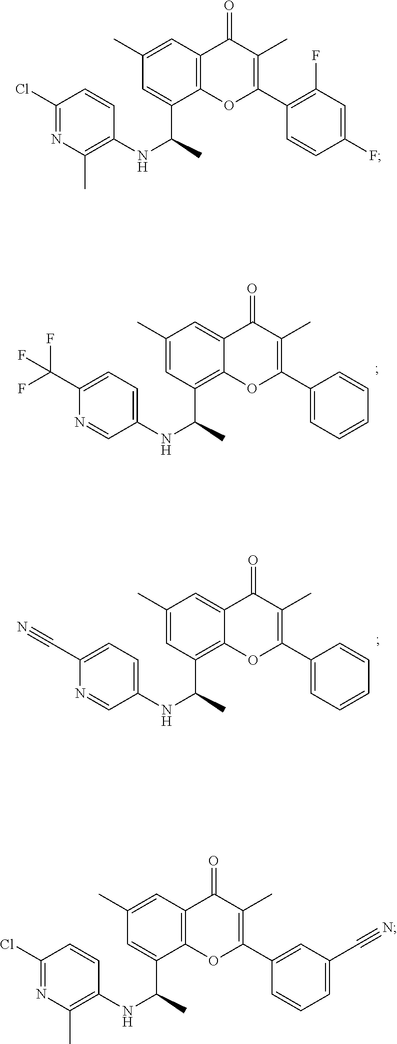 [SMILES: Cc1cc([C@@H](C)Nc2ccc(Cl)nc2C)c2oc(-c3ccc(F)cc3F)c(C)c(=O)c2c1 ; Cc1cc([C@@H](C)Nc2ccc(C(F)(F)F)nc2)c2oc(-c3ccccc3)c(C)c(=O)c2c1 ; Cc1cc([C@@H](C)Nc2ccc(C#N)nc2)c2oc(-c3ccccc3)c(C)c(=O)c2c1 ; Cc1cc([C@@H](C)Nc2ccc(Cl)nc2C)c2oc(-c3cccc(C#N)c3)c(C)c(=O)c2c1]

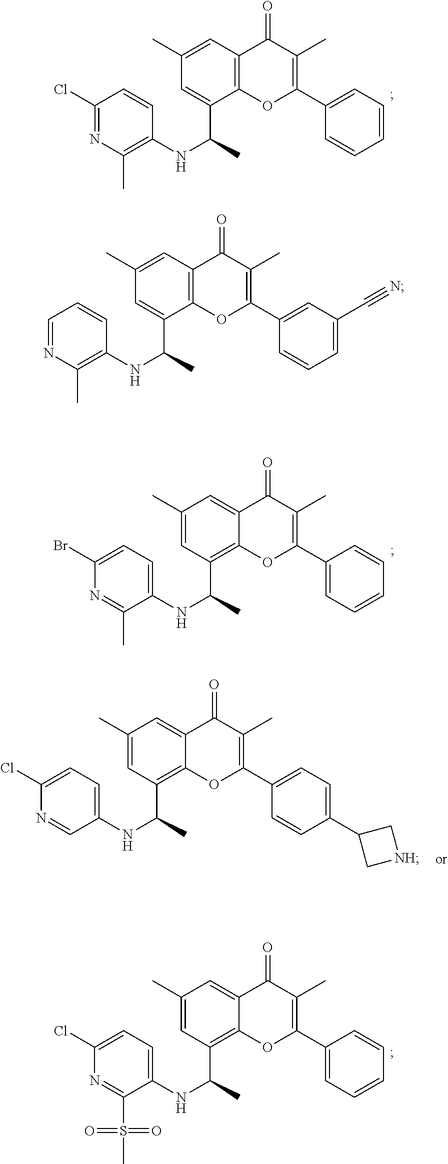 [SMILES: Cc1cc([C@@H](C)Nc2ccc(Cl)nc2C)c2oc(-c3ccccc3)c(C)c(=O)c2c1 ; Cc1cc([C@@H](C)Nc2cccnc2C)c2oc(-c3cccc(C#N)c3)c(C)c(=O)c2c1 ; Cc1cc([C@@H](C)Nc2ccc(Br)nc2C)c2oc(-c3ccccc3)c(C)c(=O)c2c1 ; Cc1cc([C@@H](C)Nc2ccc(Cl)nc2)c2oc(-c3ccc(C4CNC4)cc3)c(C)c(=O)c2c1 ; Cc1cc([C@@H](C)Nc2ccc(Cl)nc2S(C)(=O)=O)c2oc(-c3ccccc3)c(C)c(=O)c2c1]

or a pharmaceutically acceptable salt thereof.
50. The compound as defined in aspect 1 or 2 selected from:

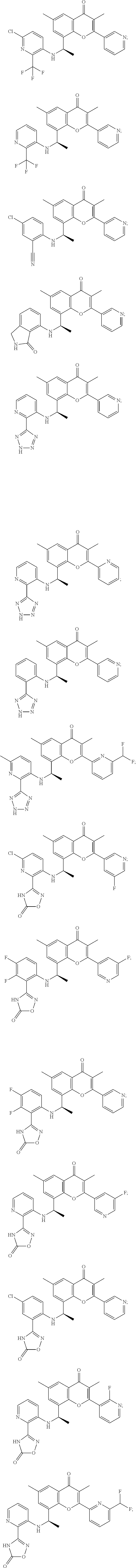

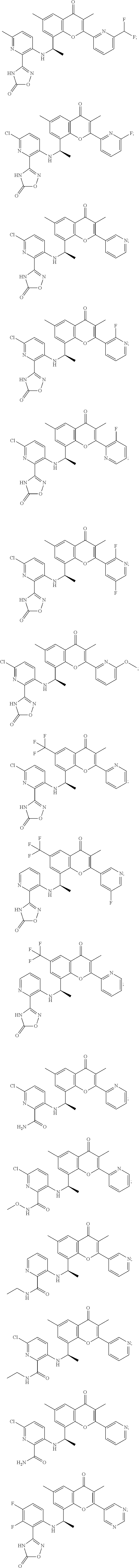

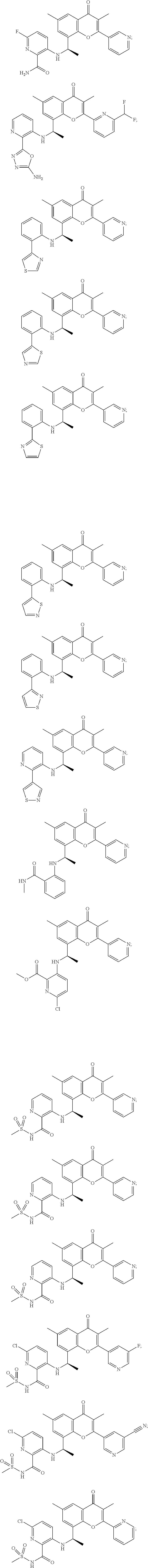

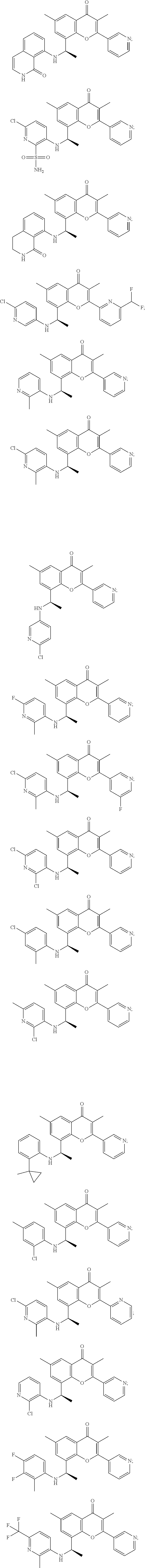

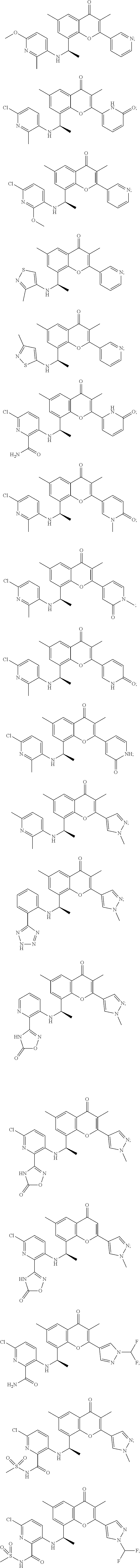

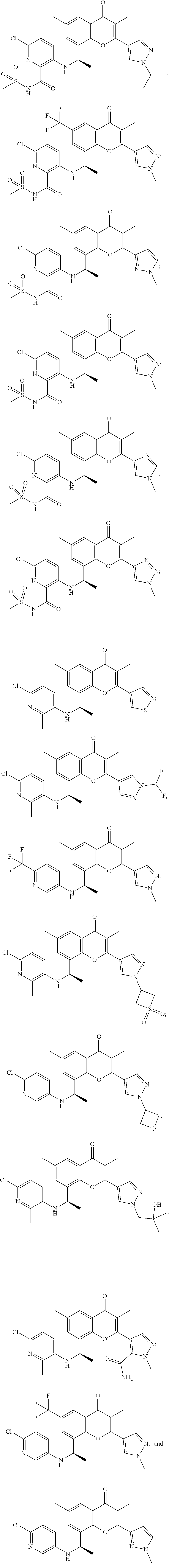

or a pharmaceutically acceptable salt thereof.
51. The compound as defined in aspect 1 or 2 selected from:

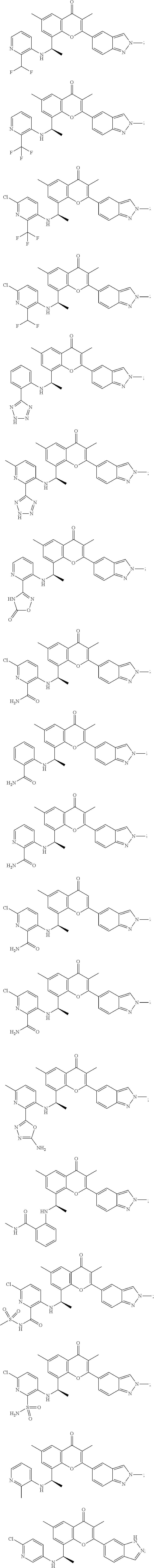

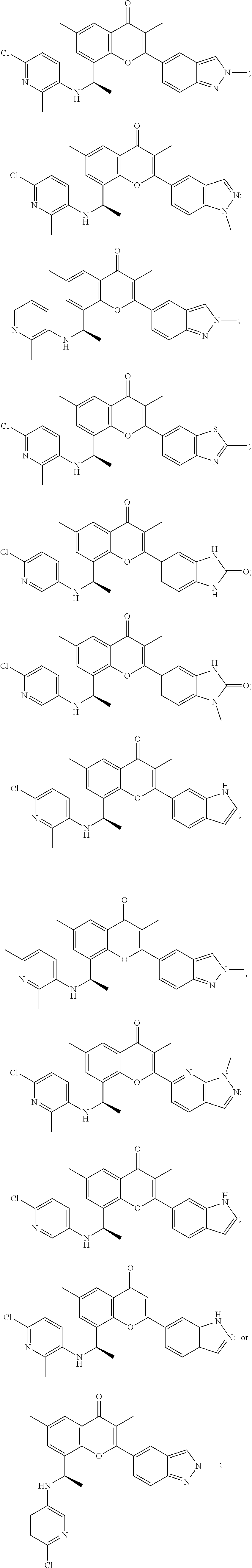

or a pharmaceutically acceptable salt thereof.
52. A pharmaceutical composition comprising a compound as defined in any one of aspects 1-51, or a pharmaceutically acceptable salt thereof, and a pharmaceutically acceptable carrier.
53. A method of treating a disease or disorder associated with modulation of phosphoinositide 3-kinase (PI3K), comprising administering to a patient in need thereof a therapeutically effective amount of a compound as defined in any one of aspects 1-51, or a pharmaceutically acceptable salt thereof, or a pharmaceutical composition as defined in aspect 52.
54. The method as defined in aspect 53, wherein the PI3K is PI3Kα.
55. The method as defined in aspect 53 or aspect 54, wherein the PI3K associated with the disease or disorder has a H1047R mutation.
56. The method as defined in any one of aspects 53-55, wherein the disease or disorder is a cancer.
57. The method as defined in aspect 56, wherein the cancer is endometrial cancer, gastric cancer, leukemia, lymphoma, sarcoma, colorectal cancer, lung cancer, ovarian cancer, skin cancer, head and neck cancer, breast cancer, brain cancer, or prostate cancer.
58. The method as defined in aspect 56, wherein the cancer is breast cancer.
59. The method as defined in aspect 56, wherein the cancer is hormone receptor-positive (HR+), human epidermal growth factor receptor 2-negative (HER2−) advanced or metastatic breast cancer.
60. The method as defined in in any one of aspects 53-55, wherein the disorder is CLOVES syndrome, or PROS.
61. A method of inhibiting phosphoinositide 3-kinase (PI3K), comprising administering to a patient in need thereof a therapeutically effective amount of a compound as defined in any one of aspects 1-51, or a pharmaceutically acceptable salt thereof, or a pharmaceutical composition as defined in aspect 52.
62. A method of treating cancer or a disorder associated with modulation of phosphoinositide 3-kinase (PI3K), the method comprising administering to a patient in need thereof a therapeutically effective amount of a compound as defined in any one of aspects 1-51, or a pharmaceutically acceptable salt thereof, or a pharmaceutical composition as defined in aspect 52.
63. The method as defined in aspect 62, wherein the cancer is endometrial cancer, gastric cancer, leukemia, lymphoma, sarcoma, colorectal cancer, lung cancer, ovarian cancer, skin cancer, head and neck cancer, breast cancer, brain cancer, or prostate cancer.
64. The method as defined in aspect 62, wherein the cancer is breast cancer.
65. The method as defined in aspect 62, wherein the cancer is hormone receptor-positive (HR+), human epidermal growth factor receptor 2-negative (HER2−) advanced or metastatic breast cancer.
66. The method as defined in aspect 62, wherein the disorder is CLOVES syndrome or PROS.
67. The compound as defined in any one of aspects 1-51, or pharmaceutically acceptable salt thereof, for use in therapy.
68. The compound as defined in any one of aspects 1-51, or a pharmaceutically acceptable salt thereof, for use in the treatment of a disease or disorder associated with modulating PI3K.
69. The compound or a pharmaceutically acceptable salt thereof for use as defined in aspect 68, wherein the disease or disorder associated with modulating PI3K is a cancer.
70. The compound or a pharmaceutically acceptable salt thereof for use as defined in aspect 69, wherein the cancer is endometrial cancer, gastric cancer, leukemia, lymphoma, sarcoma, colorectal cancer, lung cancer, ovarian cancer, skin cancer, head and neck cancer, breast cancer, brain cancer, or prostate cancer.
71. The compound or a pharmaceutically acceptable salt thereof for use as defined in aspect 69, wherein the cancer is breast cancer.
72. The compound or a pharmaceutically acceptable salt thereof for use as defined in aspect 69, wherein the cancer is hormone receptor-positive (HR+), human epidermal growth factor receptor 2-negative (HER2−) advanced or metastatic breast cancer.
73. The compound or a pharmaceutically acceptable salt thereof for use as defined in aspect 68, wherein the disorder is CLOVES syndrome or PROS.
74. Use of a compound as defined in any one of aspects 1-51, or a pharmaceutically acceptable salt thereof, in the manufacture of a medicament for the treatment of a disease or disorder associated with modulating PI3K.
75. The use as defined in aspect 74, wherein the disease or disorder associated with modulating PI3K is a cancer.
76. The use as defined in aspect 75, wherein the cancer is endometrial cancer, gastric cancer, leukemia, lymphoma, sarcoma, colorectal cancer, lung cancer, ovarian cancer, skin cancer, head and neck cancer, breast cancer, brain cancer, or prostate cancer.
77. The use as defined in aspect 75, wherein the cancer is breast cancer.
78. The use as defined in aspect 75, wherein the cancer is hormone receptor-positive (HR+), human epidermal growth factor receptor 2-negative (HER2−) advanced or metastatic breast cancer.
79. The use as defined in aspect 74, wherein the disorder is CLOVES syndrome or PROS.

### Pharmaceutical Compositions

In some aspects, the present disclosure provides a pharmaceutical composition comprising a compound of Formula (I), (II), or (III) as an active ingredient. In some embodiments, the present disclosure provides a pharmaceutical composition comprising a compound of Formula (I), (II), or (III), or a pharmaceutically acceptable salt thereof, and one or more pharmaceutically acceptable carriers or excipients.

As used herein, the term “composition” is intended to encompass a product comprising the specified ingredients in the specified amounts, as well as any product which results, directly or indirectly, from combination of the specified ingredients in the specified amounts.

The compounds of Formula (I), (II), or (III) can be formulated for oral administration in forms such as tablets, capsules (each of which includes sustained release or timed release formulations), pills, powders, granules, elixirs, tinctures, suspensions, syrups, and emulsions. The compounds of Formula (I), (II), or (III) can also be formulated for intravenous (bolus or in-fusion), intraperitoneal, topical, subcutaneous, intramuscular, or transdermal (e.g., patch) administration, all using forms well known to those of ordinary skill in the pharmaceutical arts.

The formulation of the present disclosure may be in the form of an aqueous solution comprising an aqueous vehicle. The aqueous vehicle component may comprise water and at least one pharmaceutically acceptable excipient. Suitable acceptable excipients include those selected from the group consisting of a solubility enhancing agent, chelating agent, preservative, tonicity agent, viscosity/suspending agent, buffer, and pH modifying agent, and a mixture thereof.

According to a further aspect of the disclosure there is provided a pharmaceutical composition which comprises a compound any one of the Formulae disclosed herein, or a pharmaceutically acceptable salt, in association with a pharmaceutically acceptable diluent or carrier.

The compositions of the disclosure may be in a form suitable for oral use (for example as tablets, lozenges, hard or soft capsules, aqueous or oily suspensions, emulsions, dispersible powders or granules, syrups or elixirs), for topical use (for example as creams, ointments, gels, or aqueous or oily solutions or suspensions), for administration by inhalation (for example as a finely divided powder or a liquid aerosol), for administration by insufflation (for example as a finely divided powder) or for parenteral administration (for example as a sterile aqueous or oily solution for intravenous, subcutaneous, intramuscular, intraperitoneal or intramuscular dosing or as a suppository for rectal dosing).

The compositions of the disclosure may be obtained by conventional procedures using conventional pharmaceutical excipients, well known in the art. Thus, compositions intended for oral use may contain, for example, one or more coloring, sweetening, flavoring and/or preservative agents.

### Methods of Use

In some aspects, the present disclosure provides a method of modulating PI3K (e.g., PI3Kα) activity (e.g., in vitro or in vivo), comprising contacting a cell with a therapeutically effective amount of a compound of Formula (I), (II), or (III), or a pharmaceutically acceptable salt thereof.

In some aspects, the present disclosure provides a method of treating or preventing a disease or disorder disclosed herein in a subject in need thereof, comprising administering to the subject a therapeutically effective amount of a compound of Formula (I), (II), or (III), or a pharmaceutically acceptable salt thereof, or a pharmaceutical composition of the present disclosure.

In some aspects, the present disclosure provides a method of treating a disease or disorder disclosed herein in a subject in need thereof, comprising administering to the subject a therapeutically effective amount of a compound of Formula (I), (II), or (III), or a pharmaceutically acceptable salt thereof, or a pharmaceutical composition of the present disclosure.

In some aspects, the present disclosure provides a method of treating a disease or disorder disclosed herein in a subject in need thereof, comprising administering to the subject a therapeutically effective amount of a compound of Formula (I), (II), or (III), or a pharmaceutically acceptable salt thereof, in combination with an effective amount of one or more therapeutic agents.

In some aspects, the present disclosure provides a method of treating a disease or disorder disclosed herein in a subject in need thereof, comprising administering to the subject a therapeutically effective amount of a compound of Formula (I), (II), or (III), or a pharmaceutically acceptable salt thereof, in combination with an effective amount of: a CDK4 and 6 inhibitor, or a pharmaceutically acceptable salt thereof; a SERD, or a pharmaceutically acceptable salt thereof; an aromatase inhibitor, or a pharmaceutically acceptable salt thereof; a taxane, or a pharmaceutically acceptable salt thereof; an mTOR inhibitor, or a pharmaceutically acceptable salt thereof; a tyrosine kinase inhibitor, or a pharmaceutically acceptable salt thereof, a platinum agent; an anthracycline, or a pharmaceutically acceptable salt thereof; an immune checkpoint inhibitor, or a pharmaceutically acceptable salt thereof; an antiandrogen, or a pharmaceutically acceptable salt thereof; an anti-HER2 monoclonal antibody; an anti-HER2 antibody-drug conjugate; a KRAS inhibitor, or a pharmaceutically acceptable salt thereof; an MEK inhibitor, or a pharmaceutically acceptable salt thereof, an ERK inhibitor, or a pharmaceutically acceptable salt thereof; a topoisomerase inhibitor, or a pharmaceutically acceptable salt thereof; a SERM, or a pharmaceutically acceptable salt thereof, or a PARP inhibitor, or a pharmaceutically acceptable salt thereof; or a combination thereof.

In some embodiments, the disease or disorder is associated with an implicated PI3K activity. In some embodiments, the disease or disorder is a disease or disorder in which PI3K activity is implicated.

In some embodiments, the disease or disorder is a cancer.

In some embodiments, the cancer is selected from acute lymphoblastic leukemia (ALL), acute myeloid leukemia (AML), adrenocortical carcinoma, aids-related cancers, aids-related lymphoma, anal cancer, astrocytoma, basal cell carcinoma, bile duct cancer, bladder cancer, bone cancer, osteosarcoma, malignant fibrous histiocytoma, brain tumors, breast cancer, bronchial tumors, Burkitt lymphoma, carcinoid tumor, cancer of unknown primary, cardiac (heart) tumors, atypical teratoid/rhabdoid tumor, primary CNS lymphoma, cervical cancer, cholangiocarcinoma, chordoma, chronic lymphocytic leukemia (CLL), chronic myelogenous leukemia (CML), colorectal cancer, craniopharyngioma, cutaneous t-cell lymphoma, mycosis fungoides, Sezary syndrome, ductal carcinoma in situ (DCIS), embryonal tumors, medulloblastoma, endometrial cancer, ependymoma, esophageal cancer, esthesioneuroblastoma, Ewing sarcoma, extracranial germ cell tumor, extragonadal germ cell tumor, fallopian tube cancer, gallbladder cancer, gastric cancer, gastrointestinal carcinoid tumor, malignant gastrointestinal stromal tumors (GIST), germ cell tumors, gestational trophoblastic disease, hairy cell leukemia, head and neck cancer, hepatocellular cancer, Langerhans cell histiocytosis, Hodgkin lymphoma, islet cell tumors, pancreatic neuroendocrine tumors, Kaposi sarcoma, kidney cancer, laryngeal cancer, leukemia, liver cancer, lung cancer, lymphoma, male breast cancer, intraocular melanoma, Merkel cell carcinoma, malignant mesothelioma, metastatic cancer, metastatic squamous neck cancer, midline tract carcinoma with nut gene changes, mouth cancer, multiple endocrine neoplasia syndromes, multiple myeloma/plasma cell neoplasms, myelodysplastic syndromes, myelodysplastic neoplasms, myeloproliferative neoplasms, chronic myeloproliferative neoplasm, nasal cavity and paranasal sinus cancer, nasopharyngeal cancer, neuroblastoma, non-Hodgkin lymphoma, non-small cell lung cancer, oral cancer, lip and oral cavity cancer, oropharyngeal cancer, malignant fibrous histiocytoma of bone, ovarian cancer, pancreatic cancer, pancreatic neuroendocrine tumors (islet cell tumors), papillomatosis, paraganglioma, paranasal sinus and nasal cavity cancer, parathyroid cancer, penile cancer, pharyngeal cancer, pheochromocytoma, pituitary tumor, plasma cell neoplasm, multiple myeloma, pleuropulmonary blastoma, primary central nervous system (CNS) lymphoma, primary peritoneal cancer, prostate cancer, rectal cancer, recurrent cancer, renal cell (kidney) cancer, retinoblastoma, rhabdomyosarcoma, salivary gland cancer, sarcoma, childhood vascular tumors, skin cancer, small cell lung cancer, small intestine cancer, soft tissue sarcoma, squamous cell carcinoma of the skin, testicular cancer, oropharyngeal cancer, hypopharyngeal cancer, thymoma, thymic carcinoma, thyroid cancer, tracheobronchial tumors, transitional cell cancer of the renal pelvis and ureter, urethral cancer, uterine sarcoma, vaginal cancer, vascular tumors, vulvar cancer, and Wilms tumor.

In some embodiments, the cancer is Endometrial cancer, Breast cancer, Oesophageal squamous-cell cancer, Cervical squamous-cell carcinoma, Cervical adenocarcinoma, Colorectal adenocarcinoma, Bladder Urothelial Carcinoma, Glioblastoma, Ovarian cancer, Non-small-cell Lung cancer, Esophagogastric cancer, Nerve-sheath tumor, Head and neck squamous-cell carcinoma, Melanoma, Esophagogastric adenocarcinoma, Soft-tissue sarcoma, Prostate cancer, Fibrolamellar carcinoma, Hepatocellular carcinoma, Diffuse glioma, Colorectal cancer, Pancreatic cancer, Cholangiocarcinoma, B-cell lymphoma, Mesothelioma, Adrenocortical carcinoma, Renal non-clear-cell carcinoma, Renal clear-cell carcinoma, Germ-cell carcinoma, Thymic tumor, Pheochromocytoma, Miscellaneous neuroepithelial tumor, thyroid cancer, leukemia, or encapsulated glioma.

In some embodiments, the cancer is a breast cancer, a prostate cancer, or a brain cancer.

In some embodiments, the cancer is a breast cancer. In some embodiments, the cancer is a prostate cancer. In some embodiments, the cancer is a brain cancer.

In some embodiments, the breast cancer is metastatic breast cancer. In some embodiments, the breast cancer is ductal carcinoma in situ (DCIS). In some embodiments, the breast cancer is invasive ductal carcinoma. In some embodiments, the breast cancer is triple negative breast cancer. In some embodiments, the breast cancer is medullary carcinoma. In some embodiments, the breast cancer is tubular carcinoma. In some embodiments, the breast cancer is mucinous carcinoma. In some embodiments, the breast cancer is Paget disease of the breast or nipple. In some embodiments, the breast cancer is inflammatory breast cancer (IBC). In some embodiments, the breast cancer is hormone receptor-positive (HR+), human epidermal growth factor receptor 2-negative (HER2−) advanced or metastatic breast cancer.

In some embodiments, the prostate cancer is an adenocarcinoma. In some embodiments, the prostate cancer is a small cell carcinoma. In some embodiments, the prostate cancer is a neuroendocrine tumor. In some embodiments, the prostate cancer is a transitional cell carcinoma. In some embodiments, the prostate cancer is a sarcoma.

In some embodiments, the brain cancer is an acoustic neuroma. In some embodiments, the brain cancer is an astrocytoma. In some embodiments, the brain cancer is a brain metastasis. In some embodiments, the brain cancer is choroid plexus carcinoma. In some embodiments, the brain cancer is craniopharyngioma. In some embodiments, the brain cancer is an embryonal tumor. In some embodiments, the brain cancer is an ependymoma. In some embodiments, the brain cancer is a glioblastoma. In some embodiments, the brain cancer is a glioma. In some embodiments, the brain cancer is a medulloblastoma. In some embodiments, the brain cancer is a meningioma. In some embodiments, the brain cancer is an oligodendroglioma. In some embodiments, the brain cancer is a pediatric brain tumor. In some embodiments, the brain cancer is a pineoblastoma. In some embodiments, the brain cancer is a pituitary tumor.

In some embodiments, the disease or disorder associated with PI3K includes, but is not limited to, CLOVES syndrome, PROS, breast cancer, brain cancer, prostate cancer, endometrial cancer, gastric cancer, leukemia, lymphoma, sarcoma, colorectal cancer, lung cancer, ovarian cancer, skin cancer, or head and neck cancer.

In some embodiments, the diseases or disorder associated with PI3K is CLOVES syndrome.

In some embodiments, the disease or disorder associated with PI3K is PROS.

In some embodiments, the disease or disorder associated with PI3K is breast cancer, brain cancer, prostate cancer, endometrial cancer, gastric cancer, leukemia, lymphoma, sarcoma, colorectal cancer, lung cancer, ovarian cancer, skin cancer, or head and neck cancer.

In some embodiments, the disease or disorder associated with PI3K is a breast neoplasm, a thyroid neoplasm, an ovarian neoplasm, non-small-cell lung carcinoma, an endometrial neoplasm, or a pancreatic neoplasm. In some embodiments, the disease or disorder associated with PI3K is a breast neoplasm. In some embodiments, the disease or disorder associated with PI3K is a thyroid neoplasm. In some embodiments, the disease or disorder associated with PI3K is an ovarian neoplasm. In some embodiments, the disease or disorder associated with PI3K is non-small-cell lung carcinoma. In some embodiments, the disease or disorder associated with PI3K is an endometrial neoplasm. In some embodiments, the disease or disorder associated with PI3K is a pancreatic neoplasm.

In some embodiments, the disease or disorder associated with PI3K is breast cancer, brain cancer, prostate cancer, endometrial cancer, gastric cancer, colorectal cancer, lung cancer, ovarian cancer, skin cancer, or head and neck cancer.

In some embodiments, the disease or disorder associated with PI3K is leukemia, lymphoma, or sarcoma.

In some embodiments, the cancer is endometrial cancer, head and neck cancer, or a sarcoma.

In some embodiments, the cancer is endometrial cancer. In some embodiments the cancer is head and neck cancer. In some embodiments, the cancer is a sarcoma.

In some embodiments, the sarcoma is soft tissue sarcoma, osteosarcoma, chondrosarcoma, Ewing sarcoma, hemangioendothelioma, angiosarcoma, fibrosarcoma, myofibrosarcoma, chordoma, adamantinoma, liposarcoma, leiomyosarcoma, malignant peripheral nerve sheath tumor, rhabdomyosarcoma, synovial sarcoma, or malignant solitary fibrous tumor.

In some embodiments, the sarcoma is soft tissue sarcoma. In some embodiments the soft tissue sarcoma is liposarcoma, atypical lipomatous tumor, dermatofibrosarcoma protuberans, malignant solitary fibrous tumor, inflammatory myofibroblastic tumor, low-grade myofibroblastic sarcoma, fibrosarcoma, myxofibrosarcoma, low-grade fibromyxoid sarcoma, giant cell tumor of soft tissues, leiomyosarcoma, malignant glomus tumor, rhabdomyosarcoma, hemangioendothelioma, angiosarcoma of soft tissue, extraskeletal osteosarcoma, gastrointestinal stromal tumor, malignant gastrointestinal stromal tumor (GIST), malignant peripheral nerve sheath tumor, malignant Triton tumor, malignant granular cell tumor, malignant ossifying fibromyxoid tumor, stromal sarcoma, myoepithelial carcinoma, malignant phosphaturic mesenchymal tumor, synovial sarcoma, epithelioid sarcoma, alveolar soft part sarcoma, clear cell sarcoma of soft tissue, extraskeletal myxoid chondrosarcoma, extraskeletal Ewing sarcoma, desmoplastic small round cell tumor, extrarenal rhabdoid tumor, perivascular epithelioid cell tumor, intimal sarcoma, undifferentiated spindle cell sarcoma, undifferentiated pleomorphic sarcoma, undifferentiated round cell sarcoma, undifferentiated epithelioid sarcoma, or undifferentiated sarcoma, not otherwise specified.

In some aspects, the present disclosure provides a method of treating or preventing a cancer in a subject in need thereof, comprising administering to the subject a therapeutically effective amount of a compound of Formula (I), (II), or (III), or a pharmaceutically acceptable salt thereof, or a pharmaceutical composition of the present disclosure.

In some aspects, the present disclosure provides a method of treating a cancer in a subject in need thereof, comprising administering to the subject a therapeutically effective amount of a compound of Formula (I), (II), or (III), or a pharmaceutically acceptable salt thereof, or a pharmaceutical composition of the present disclosure.

In some aspects, the present disclosure provides a method of treating or preventing a breast cancer in a subject in need thereof, comprising administering to the subject a therapeutically effective amount of a compound of Formula (I), (II), or (III), or a pharmaceutically acceptable salt thereof, or a pharmaceutical composition of the present disclosure.

In some aspects, the present disclosure provides a method of treating a breast cancer in a subject in need thereof, comprising administering to the subject a therapeutically effective amount of a compound of Formula (I), (II), or (III), or a pharmaceutically acceptable salt thereof, or a pharmaceutical composition of the present disclosure.

In some aspects, the present disclosure provides a method of treating or preventing a prostate cancer in a subject in need thereof, comprising administering to the subject a therapeutically effective amount of a compound of Formula (I), (II), or (III), or a pharmaceutically acceptable salt thereof, or a pharmaceutical composition of the present disclosure.

In some aspects, the present disclosure provides a method of treating a prostate cancer in a subject in need thereof, comprising administering to the subject a therapeutically effective amount of a compound of Formula (I), (II), or (III), or a pharmaceutically acceptable salt thereof, or a pharmaceutical composition of the present disclosure.

In some aspects, the present disclosure provides a method of treating or preventing a brain cancer in a subject in need thereof, comprising administering to the subject a therapeutically effective amount of a compound of Formula (I), (II), or (III), or a pharmaceutically acceptable salt thereof, or a pharmaceutical composition of the present disclosure.

In some aspects, the present disclosure provides a method of treating a brain cancer in a subject in need thereof, comprising administering to the subject a therapeutically effective amount of a compound of Formula (I), (II), or (III), or a pharmaceutically acceptable salt thereof, or a pharmaceutical composition of the present disclosure.

In some aspects, the present disclosure provides a compound of Formula (I), (II), or (III), or a pharmaceutically acceptable salt thereof for use in therapy.

In some aspects, the present disclosure provides a compound of Formula (I), (II), or (III), or a pharmaceutically acceptable salt thereof for use in modulating PI3K (e.g., PI3Kα) activity (e.g., in vitro or in vivo).

In some aspects, the present disclosure provides a compound of Formula (I), (II), or (III), or a pharmaceutically acceptable salt thereof for use in treating or preventing a disease or disorder disclosed herein.

In some aspects, the present disclosure provides a compound of Formula (I), (II), or (III), or a pharmaceutically acceptable salt thereof for use in treating a disease or disorder disclosed herein.

In some aspects, the present disclosure provides a compound of Formula (I), (II), or (III), or a pharmaceutically acceptable salt thereof for use in treating or preventing a cancer.

In some aspects, the present disclosure provides a compound of Formula (I), (II), or (III), or a pharmaceutically acceptable salt thereof for use in treating a cancer.

In some aspects, the present disclosure provides a compound of Formula (I), (II), or (III), or a pharmaceutically acceptable salt thereof for use in treating or preventing a breast cancer.

In some aspects, the present disclosure provides a compound of Formula (I), (II), or (III), or a pharmaceutically acceptable salt thereof for use in treating a breast cancer.

In some aspects, the present disclosure provides a compound of Formula (I), (II), or (III), or a pharmaceutically acceptable salt thereof for use in treating or preventing a prostate cancer.

In some aspects, the present disclosure provides a compound of Formula (I), (II), or (III), or a pharmaceutically acceptable salt thereof for use in treating a prostate cancer.

In some aspects, the present disclosure provides a compound of Formula (I), (II), or (III), or a pharmaceutically acceptable salt thereof for use in treating or preventing a brain cancer.

In some aspects, the present disclosure provides a compound of Formula (I), (II), or (III), or a pharmaceutically acceptable salt thereof for use in treating a brain cancer.

In some aspects, the present disclosure provides use of a compound of Formula (I), (II), or (III), or a pharmaceutically acceptable salt thereof in the manufacture of a medicament for modulating PI3K (e.g., PI3Kα) activity (e.g., in vitro or in vivo).

In some aspects, the present disclosure provides use of a compound of Formula (I), (II), or (III), or a pharmaceutically acceptable salt thereof in the manufacture of a medicament for treating or preventing a disease or disorder disclosed herein.

In some aspects, the present disclosure provides use of a compound of Formula (I), (II), or (III), or a pharmaceutically acceptable salt thereof in the manufacture of a medicament for treating a disease or disorder disclosed herein.

In some aspects, the present disclosure provides use of a compound of Formula (I), (II), or (III), or a pharmaceutically acceptable salt thereof in the manufacture of a medicament for treating or preventing a cancer in a subject in need thereof.

In some aspects, the present disclosure provides use of a compound of Formula (I), (II), or (III), or a pharmaceutically acceptable salt thereof in the manufacture of a medicament for treating a cancer in a subject in need thereof.

In some aspects, the present disclosure provides use of a compound of Formula (I), (II), or (III), or a pharmaceutically acceptable salt thereof in the manufacture of a medicament for treating or preventing a breast cancer in a subject in need thereof.

In some aspects, the present disclosure provides use of a compound of Formula (I), (II), or (III), or a pharmaceutically acceptable salt thereof in the manufacture of a medicament for treating a breast cancer in a subject in need thereof.

In some aspects, the present disclosure provides use of a compound of Formula (I), (II), or (III), or a pharmaceutically acceptable salt thereof in the manufacture of a medicament for treating or preventing a prostate cancer in a subject in need thereof.

In some aspects, the present disclosure provides use of a compound of Formula (I), (II), or (III), or a pharmaceutically acceptable salt thereof in the manufacture of a medicament for treating a prostate cancer in a subject in need thereof.

In some aspects, the present disclosure provides use of a compound of Formula (I), (II), or (III), or a pharmaceutically acceptable salt thereof in the manufacture of a medicament for treating or preventing a brain cancer in a subject in need thereof.

In some aspects, the present disclosure provides use of a compound of Formula (I), (II), or (III), or a pharmaceutically acceptable salt thereof in the manufacture of a medicament for treating a brain cancer in a subject in need thereof.

The present disclosure provides compounds that function as modulators of PI3K activity. The present disclosure therefore provides a method of modulating PI3K activity in vitro or in vivo, said method comprising contacting a cell with a therapeutically effective amount of a compound, or a pharmaceutically acceptable salt thereof, as defined herein.

In some embodiments, PI3K modulation is inhibition of PI3K.

In some embodiments, the PI3K inhibitor is a PI3Kα inhibitor. In some embodiments, the PI3K inhibitor is a PI3Kα H1047R mutant inhibitor.

Effectiveness of compounds of the disclosure can be determined by industry-accepted assays/disease models according to standard practices of elucidating the same as described in the art and are found in the current general knowledge.

The present disclosure also provides a method of treating a disease or disorder in which PI3K activity is implicated in a patient in need of such treatment, said method comprising administering to said patient a therapeutically effective amount of a compound, or a pharmaceutically acceptable salt thereof, or a pharmaceutical composition as defined herein.

### Routes of Administration

The compounds of Formula (I), (II), or (III), or pharmaceutical compositions comprising these compounds may be administered to a subject by any convenient route of administration, whether systemically/peripherally or topically (i.e., at the site of desired action).

Routes of administration include, but are not limited to, oral (e.g. by ingestion); buccal; sublingual; transdermal (including, e.g., by a patch, plaster, etc.); transmucosal (including, e.g., by a patch, plaster, etc.); intranasal (e.g., by nasal spray); ocular (e.g., by eye drops); pulmonary (e.g., by inhalation or insufflation therapy using, e.g., via an aerosol, e.g., through the mouth or nose); rectal (e.g., by suppository or enema); vaginal (e.g., by pessary); parenteral, for example, by injection, including subcutaneous, intradermal, intramuscular, intravenous, intra-arterial, intracardiac, intrathecal, intraspinal, intracapsular, subcapsular, intraorbital, intraperitoneal, intratracheal, subcuticular, intraarticular, subarachnoid, and intrasternal; by implant of a depot or reservoir, for example, subcutaneously or intramuscularly.

### EXAMPLES

Exemplary compounds of Formula (I), (II), and (III) are synthesized and tested in the examples. It is understood that compounds of Formula (I), (II), and (III) may be converted to the corresponding pharmaceutically acceptable salts of the compounds using routine techniques in the art.

Nuclear magnetic resonance (NMR) spectra were recorded at 400 MHz or 300 MHz as stated and at 300.3 K unless otherwise stated; the chemical shifts (6) are reported in parts per million (ppm). Spectra were recorded using a Bruker or Varian instrument with 8, 16 or 32 scans.

LC-MS chromatograms and spectra were recorded using an Agilent 1200 or Shimadzu LC-20 AD&MS 2020 instrument using a C-18 column such as a Luna-C18 2.0×30 mm or Xbridge Shield RPC18 2.1×50 mm. Injection volumes were 0.7-8.0 μl and the flow rates were typically 0.8 or 1.2 ml/min. Detection methods were diode array (DAD) or evaporative light scattering (ELSD) as well as positive ion electrospray ionization. MS range was 100-1000 Da. Solvents were gradients of water and acetonitrile both containing a modifier (typically 0.01-0.04%) such as trifluoroacetic acid or ammonium carbonate.

### Abbreviations

ACN Acetonitrile
AcOH Acetic Acid
ADP Adenosine diphosphate
ATP Adenosine triphosphate
CDCl₃ Chloroform-d
CPhos 2-Dicyclohexylphosphino-2′,6′-bis(N,N-dimethylamino)biphenyl
DBU 1,8-diazabicyclo[5.4.0]undec-7-ene
DCM Dichloromethane
DIEA N,N-diisopropylethylamine
DIPEA N,N-diisopropylethylamine
DMA N,N-dimethylacetamide
DMAP Dimethylaminopyridinde
DMEA Dimethylethylamine
DMF N,N-dimethylformamide
DMSO Dimethylsulfoxide
DMSO-d₆ Hexadeuterodimethylsulfoxide
EDC N-(3-Dimethylaminopropyl)-N′-ethylcarbodiimide
eq equivalents
EtOAc Ethyl Acetate
EtOH Ethanol
h, hr hour(s)
HCl Hydrogen chloride
¹H NMR Proton nuclear magnetic resonance spectroscopy
HATU O-(7-Azabenzotriazol-1-yl)-N,N,N′,N′-tetramethyluronium hexafluorophosphate
IPA Isopropanol
LC-MS Liquid Chromatography-Mass Spectrometry
mCPBA meta-Chloroperoxybenzoic acid
MeOH Methanol
MgSO₄ Magnesium sulfate
min minute(s)
MS ES Mass Spectroscopy Electro Spray
MTBE Methyl tert-butyl ether
NaCl Sodium chloride
NaHCO₃Sodium bicarbonate
NaHMDS Sodium bis(trimethylsilyl)amide
NaOH Sodium hydroxide
Na₂S₂O₃ Sodium thiosulfate
Na₂SO₄ Sodium sulfate
NH₄Cl Ammonium chloride
NH₄HCO₃ Ammonium bicarbonate
NH₄OH Ammonium hydroxide
NMP N-methyl-2-pyrrolidone
ppm parts per million
rt room temperature
SFC Supercritical Fluid Chromatography
TBAF Tetrabutylammonium fluoride
TFA Trifluoroacetic acid
THF Tetrahydrofuran

### Intermediate 1C: tert-Butyl 6-bromo-2,3-difluoro-benzoate

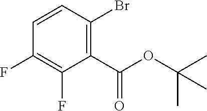 [SMILES: CC(C)(C)OC(=O)c1c(Br)ccc(F)c1F]

A solution of 6-bromo-2,3-difluoro-benzoic acid (2.9 g, 12 mmol) in THF (24 mL) was treated with tert-butyl (Z)—N,N′-diisopropylcarbamimidate (9.8 g, 49 mmol) dropwise and the resulting mixture sealed and heated at 65° C. for 18 h. The reaction was cooled to rt and diluted with MTBE. The suspension was filtered and the filtrate concentrated under reduced pressure. The residue was purified by silica gel chromatography eluted with 0% to 30% EtOAc in hexane to give the title compound (3.3 g, 92%) as a colorless oil.

### Intermediate 2C: tert-Butyl 4-(3-bromo-6-chloro-2-pyridyl)piperazine-1-carboxylate

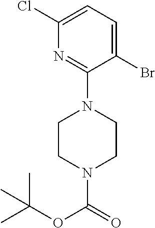 [SMILES: CC(C)(C)OC(=O)N1CCN(c2nc(Cl)ccc2Br)CC1]

A vial was charged with tert-butyl piperazine-1-carboxylate (0.43 g, 2.28 mmol), 3-bromo-6-chloro-2-fluoro-pyridine (0.20 g, 0.95 mmol), cesium carbonate (0.93 g, 2.85 mmol), and DMF (4 mL). The vial was sealed and stirred overnight at rt. The reaction was partitioned between EtOAc and water. The aqueous layer was extracted twice with EtOAc. The organic layers were combined, washed with saturated aqueous NaCl, collected, and concentrated under reduced pressure. The residue was purified by silica gel chromatography eluted with 0% to 50% EtOAc in heptane to give the title compound (0.34 g, 94%) as a colorless oil. ES/MS m/z 322 (M−^{t}Bu).

The following compounds in Table 1 were made in a similar way as described for tert-butyl 4-(3-bromo-6-chloro-2-pyridyl)piperazine-1-carboxylate. Various methods were used to purify the compounds, which would be apparent to one skilled in the art.

TABLE 1

| | | | MS ES+ |
|---|---|---|---|
| Interm # | Chemical Name | Structure | m/z |
| 3C | 6-(3-Bromo-6-chloro-2-pyridyl)-2-oxa-6- azaspiro[3.3]heptane | 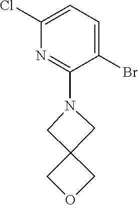 [SMILES: Clc1ccc(Br)c(N2CC3(COC3)C2)n1] | 289/291 [M + H]⁺ |
| 4C¹ | 4-(3-Bromo-6-chloro-2-pyridyl)morpholine | 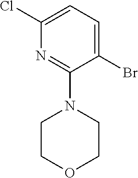 [SMILES: Clc1ccc(Br)c(N2CCOCC2)n1] | 277/279 [M + H]⁺ |
| 5C¹ | [1-(3-Bromo-6-chloro-2-pyridyl)-4- piperidyl]oxy-tert-butyl-dimethyl-silane | 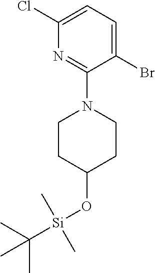 [SMILES: CC(C)(C)[Si](C)(C)OC1CCN(c2nc(Cl)ccc2Br)CC1] | 405/407 [M + H]⁺ |
| 6C¹ | 2-(Azetidin-1-yl)-3-bromo-6-chloro-pyridine | 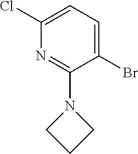 [SMILES: Clc1ccc(Br)c(N2CCC2)n1] | 247/249 [M + H]⁺ |
| 7C¹ | [(3S,4S)-1-(3-Bromo-6-chloro-2-pyridyl)-4- [tert-butyl(dimethyl)silyl]oxy-pyrrolidin-3- yl]oxy-tert-butyl-dimethyl-silane | 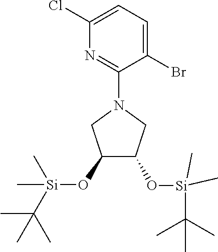 [SMILES: CC(C)(C)[Si](C)(C)O[C@H]1CN(c2nc(Cl)ccc2Br)C[C@@H]1O[Si](C)(C)C(C)(C)C] | 409 [M − TBDMS]⁺ |
| 8C¹ | 6-(3-Bromo-6-chloro-2-pyridyl)-1-oxa-6- azaspiro[3.3]heptane | 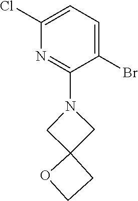 [SMILES: Clc1ccc(Br)c(N2CC3(CCO3)C2)n1] | 289/291 [M + H]⁺ |
| 9C¹ | 4-[1-(3-Bromo-6-chloro-2-pyridyl)azetidin- 3-yl]morpholine | 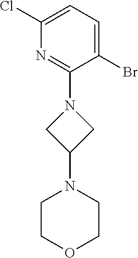 [SMILES: Clc1ccc(Br)c(N2CC(N3CCOCC3)C2)n1] | 332/334 [M + H]⁺ |
| 10C¹ | 3-Bromo-6-chloro-2-(3,3-difluoroazetidin-1- yl)pyridine | 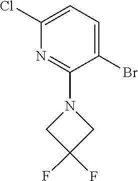 [SMILES: FC1(F)CN(c2nc(Cl)ccc2Br)C1] | 281/283 [M + H]⁺ |
| 11C¹ | 2-(3-Bromo-6-chloro-2-pyridyl)-7-oxa-2- azaspiro[3.5]nonane | 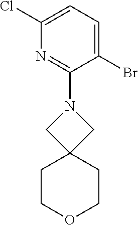 [SMILES: Clc1ccc(Br)c(N2CC3(CCOCC3)C2)n1] | 317/319 [M + H]⁺ |
| 12C¹ | [1-(3-Bromo-6-chloro-2-pyridyl)azetidin-3- yl]oxy-tert-butyl-dimethyl-silane | 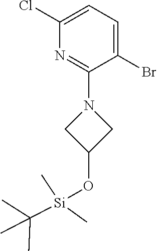 [SMILES: CC(C)(C)[Si](C)(C)OC1CN(c2nc(Cl)ccc2Br)C1] | 377/379 [M + H]⁺ |
| 13C¹ | 1-(3-Bromo-6-chloro-2-pyridyl)azetidine-3- carbonitrile | 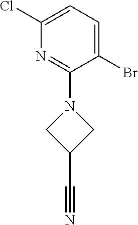 [SMILES: N#CC1CN(c2nc(Cl)ccc2Br)C1] | 272/274 [M + H]⁺ |
| 14C¹ | 1-(3-Bromo-6-chloro-2-pyridyl)-4-(oxetan-3- yl)piperazine | 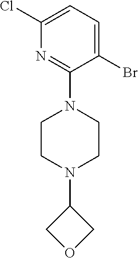 [SMILES: Clc1ccc(Br)c(N2CCN(C3COC3)CC2)n1] | 332/334 [M + H]⁺ |
| 15C¹ | 4-(3-Bromo-6-chloro-2-pyridyl)-1,4- oxazepane | 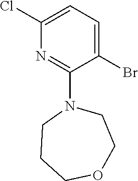 [SMILES: Clc1ccc(Br)c(N2CCCOCC2)n1] | 291/293 [M + H]⁺ |
| 16C¹ | 1-(3-Bromo-6-chloro-2-pyiridyl)piperidin-4- carbonitrile | 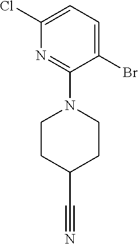 [SMILES: N#CC1CCN(c2nc(Cl)ccc2Br)CC1] | 300/302 [M + H]⁺ |

¹DIPEA used as the base.

### Intermediate 17C: 2-Fluoro-N-methoxy-benzenesulfonamide

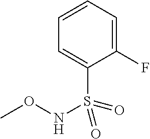

Combined 2-fluorobenzenesulfonyl chloride (0.70 g, 3.60 mmol) and potassium carbonate (1.09 g, 7.91 mmol) in EtOH (5 mL) and water (5 mL) and allowed to stir at rt overnight. The reaction was diluted with water (20 mL) and the title compound removed by filtration (0.40 g, 54%). ES/MS m/z 206 (M+H).

### Intermediate 18C: (1,5-Dimethyl-6-oxo-3-pyridyl)boronic acid

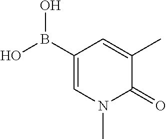

A solution of 1,3-dimethyl-5-(4,4,5,5-tetramethyl-1,3,2-dioxaborolan-2-yl)pyridin-2-one (4 g, 16.06 mmol) in acetone (40 mL) and water (20 mL) was treated dropwise with concentrated aqueous HCl (20 mL, 36.5%) at rt under a nitrogen atmosphere. The reaction was stirred for 3 h at rt. The reaction was basified to pH 8 with NH₄OH and concentrated under reduced pressure. The residue was purified by reversed phase chromatography on C18 eluted with 40% to 60% ACN in water (with 0.1% NH₄OH) to give the title compound (2.0 g, 75%) as an off-white solid. ES/MS m/z 168 (M+H).

### Intermediate 19C: 1-[5-(4,4,5,5-Tetramethyl-1,3,2-dioxaborolan-2-yl)-2-pyridyl]cyclopropanecarbonitrile

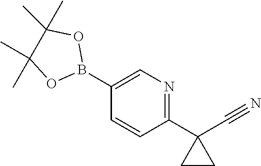 [SMILES: CC1(C)OB(c2ccc(C3(C#N)CC3)nc2)OC1(C)C]

A solution of 1-(5-bromo-2-pyridyl)cyclopropanecarbonitrile (0.21 g, 0.94 mmol), bis(pinacolato)diborane (0.36 g, 1.41 mmol), and potassium acetate (0.28 g, 2.82 mmol) in dry 1,4-dioxane (10 mL) was purged with argon for 10 min. Added [1,1′-bis(diphenylphosphino)ferrocene]dichloropalladium(II) (69 mg, 0.09 mmol) and purged the reaction an additional 3 min with argon. The reaction was stirred at 100° C. for 2 h. After cooling to rt, the reaction was filtered through diatomaceous earth and the filtrate concentrated under reduced pressure. The residue was purified by silica gel chromatography eluted with 0% to 20% MeOH in DCM to give the title compound (0.35 g, 83%, 60% purity). ES/MS m/z 189 (M+H).

The following compound in Table 2 was made in a similar way as described for 1-[5-(4,4,5,5-tetramethyl-1,3,2-dioxaborolan-2-yl)-2-pyridyl]cyclopropanecarbonitrile. Various methods were used to purify this compound, which would be apparent to one skilled in the art.

TABLE 2

| Interm | | | MS ES+ |
|---|---|---|---|
| # | Chemical Name | Structure | m/z |
| 20C | 6-(4,4,5,5- Tetramethyl-1,3,2- dioxaborolan-2-yl)- 1H-indole-3- carboxylic acid | 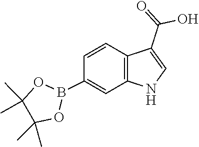 [SMILES: CC1(C)OB(c2ccc3c(C(=O)O)c[nH]c3c2)OC1(C)C] | 288 [M + H]⁺ |

### Intermediate 21C: Tributyl-(3,6-difluoro-2-pyridyl)stannane

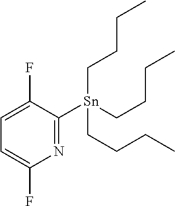 [SMILES: CCC[CH2][Sn]([CH2]CCC)([CH2]CCC)[c]1nc(F)ccc1F]

A solution of 2,5-difluoropyridine (2.0 g, 17.38 mmol) in THF (10 mL) at −78° C. was treated with lithium diisopropylamide solution (1M in THF, 2.79 g, 26.07 mmol). The reaction was stirred at −78° C. for 1 h. Tributyltin chloride (5.94 g, 18.25 mmol) in 20 mL of THF was added dropwise to the reaction at −78° C. and then the reaction stirred for 1 h at −78° C. The reaction was quenched with aqueous NH₄Cl and extracted with EtOAc (3×). The combined extracts were dried over Na₂SO₄, filtered, and the reaction concentrated under reduced pressure. The residue was purified by silica gel chromatography eluted with 0% to 20% EtOAc in heptane to give the title compound (4.46 g, 64%). ES/MS m/z 406 (M+H).

### Intermediate 22C: 3-Bromo-N-tert-butyl-pyridine-2-sulfonamide

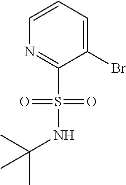

A solution of 3-bromopyridine-2-sulfonyl chloride (2.0 g, 7.80 mmol) in DCM was cooled to −20° C. and treated dropwise with tert-butylamine. The reaction was stirred at −20° C. for 30 min. The reaction was concentrated under reduced pressure and the residue purified by silica gel chromatography eluted with 50% EtOAc in petroleum ether to give the title compound (0.50 g, 22%) as a dark red solid. ES/MS m/z 293/295 (M+H).

### Intermediate 23C: 5-Chloro-2-methyl-pyrazolo[4,3-b]pyridine

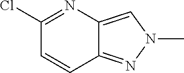

A mixture of 5-chloro-1H-pyrazolo[4,3-b]pyridine (3.2 g, 20.84 mmol) and iodomethane (4.44 g, 31.26 mmol) in THF (30 mL) at 0° C. was treated in portions with sodium hydride (1.0 g, 60% in oil, 25.0 mmol). The reaction was stirred at rt for 1 h, quenched with water (30 mL), and extracted with EtOAc (3×80 mL). The combined organic layers were washed with saturated aqueous sodium chloride (3×30 mL), dried over Na₂SO₄, filtered, and concentrated under reduced pressure. The residue was purified by silica gel chromatography eluted with 30% EtOAc in petroleum ether to give the title compound (1.34 g, 38%) as a white solid. ES/MS m/z 168 (M+H).

### Intermediate 24C: Trimethyl-(2-methylpyrazolo[4,3-b]pyridin-5-yl)stannane

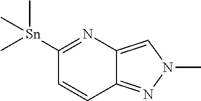

A solution of 5-chloro-2-methyl-pyrazolo[4,3-b]pyridine (4.0 g, 23.87 mmol) in 1,4-dioxane (80 mL) was treated with hexamethylditin (11.73 g, 35.8 mmol) and tetrakis(triphenylphosphine)palladium(0) (5.52 g, 4.77 mmol). The reaction was stirred for 2 h at 100° C. The reaction was cooled to rt, quenched with saturated aqueous potassium fluoride, filtered, and the solids washed with EtOAc (3×100 mL). The filtrate was concentrated under reduced pressure to give the title compound (6.0 g) as a black solid. ES/MS m/z 298 (M+H).

### Intermediate 25C: Iodo-[3-(trifluoromethyl)-1-bicyclo[1.1.1]pentanyl]zinc

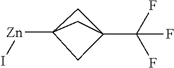

An oven dried flask equipped with stir bar and septum was charged with lithium chloride (1.62 g, 38.17 mmol) and heated under high vacuum at 170° C. for 20 min. The vacuum was removed and nitrogen gas introduced into the flask as it was cooling. Zinc (2.50 g, 38.17 mmol) was added to the flask when cool and dried under high vacuum at 170° C. for 20 min and backfilled with nitrogen as it was cooling. When cooled to rt, THF (20 mL) and 1,2-dibromoethane (0.18 g, 38.17 mmol) were added via syringe and the reaction heated at 60° C. until bubbling occurred. After cooling to rt, chlorotrimethylsilane (0.02 g, 0.19 mmol) and iodine (0.02 g, 0.1 mmol) in THF (1 mL) were added via syringe and the reaction heated at 60° C. for 20 min and then cooled to rt. Added 1-iodo-3-(trifluoromethyl)bicyclo[1.1.1]pentane (5.0 g, 19.08 mmol) and stirred the reaction at 50° C. overnight. The reaction was allowed to stand at rt for 1 h and the clear solution on top of the solids carefully transferred via cannula to a dry flask for use without purification assuming ˜0.45 M concentration.

The following compounds in Table 3 were made in a similar way as described for iodo-[3-(trifluoromethyl)-1-bicyclo[1.1.1]pentanyl]zinc.

TABLE 3

| | | | MS ES+ |
|---|---|---|---|
| Interm # | Chemical Name | Structure | m/z |
| 26C | (4,4-Difluorocyclohexyl)- iodo-zinc | 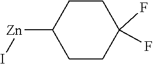 | |
| 27C | Cyclopropyl(iodo)zinc | 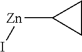 | |

### Intermediate 28C: 1-(3-Bromo-5-fluoro-2-hydroxy-phenyl)ethanone

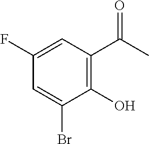

1-(5-Fluoro-2-hydroxy-phenyl)ethanone (2.0 kg, 13 mol) was dissolved in DMF (20 L) and treated with N-bromosuccinimide (2.8 kg, 15.6 mol) keeping the temperature below 20° C. Stirred at 0 to 10° C. for 5 to 6 h. Slowly added water (20 L) to the reactor, stirred at rt for 2 h, and filtered off the crude product. This material was slurried in heptane (10 L) at 0 to 25° C. for 2 h and removed the title compound (2.5 kg, 83%) by filtration as a yellow solid. ¹H NMR (400 MHz, DMSO-d6) δ ppm 2.69 (3H, s), 7.90 (2H, m), 12.53 (1H, s).

### Intermediate 1A: (2-Bromo-4-methyl-phenyl) propanoate

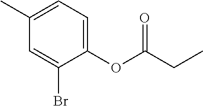

A mixture of 2-bromo-4-methyl-phenol (10.0 g, 53.5 mmol) and pyridine (6.34 g, 80.2 mmol) in DCM (100 mL) was treated with propanoyl chloride (5.44 g, 58.8 mmol) at 0° C. and stirred at 25° C. for 16 h. The mixture was diluted with water (100 mL), the pH adjusted to 5 with HCl (2 M), and extracted with DCM (2×100 mL). The combined organic extracts were washed with saturated aqueous sodium chloride (2×150 mL), dried over anhydrous Na₂SO₄, filtered, and concentrated to give the product as an oil (13 g, crude). ¹H NMR (400 MHz, DMSO-d6) δ ppm 1.17 (t, J=7.6 Hz, 3H), 2.30 (s, 3H), 2.62 (q, J=7.6 Hz, 2H), 7.11-7.18 (m, 1H), 7.19-7.26 (m, 1H), 7.50-7.55 (m, 1H).

### Intermediate 29C: (2-Bromo-4-fluoro-phenyl) propanoate

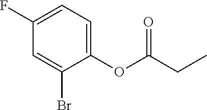

2-Bromo-4-fluoro-phenol (3.0 kg, 15.7 mol) and EtOAc (15 L) were added to a 50 L reactor and stirred for 15 min. The solution was slowly treated with triethylamine (1.9 kg, 18.8 mol). The reaction was cooled and propionyl chloride (1.6 kg, 17.3 mol) was added dropwise keeping the temperature between 0 and 10° C. After addition was completed, the reaction was stirred at rt for 15 h. The reaction was carefully diluted with water (15 L) keeping the temperature of the reaction below 30° C. and then stirred at rt for 30 min. The organic phase was separated, washed with water (10 L), collected, dried over MgSO₄, filtered, and concentrated under reduced pressure at 45° C. to give the title compound (3.6 kg, 93%) as a yellow liquid. ¹H NMR (400 MHz, DMSO-d6) δ ppm 1.15 (t, 3H), 2.65 (dd, 2H), 7.30 (m, 2H), 7.75 (m, 1H).

### Intermediate 58A: 3-Bromo-N-tert-butyl-6-chloro-pyridine-2-sulfonamide

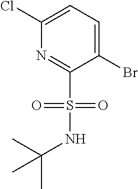 [SMILES: CC(C)(C)NS(=O)(=O)c1nc(Cl)ccc1Br]

A 0° C. solution of 3-bromo-6-chloro-pyridine-2-sulfonyl chloride (1.94 g, 6.68 mmol) in 40 mL of DCM was treated dropwise with 2-methylpropan-2-amine (3.90 g, 53.28 mmol) in 5 mL of DCM over 2 min. After addition, the reaction was allowed to stir at rt for 1 h. The reaction was diluted with 50 mL of water and extracted with DCM (3×50 mL). The combined organics were washed with saturated aqueous sodium chloride (3×50 mL), collected, dried over Na₂SO₄, filtered, and concentrated. The residue was purified by silica gel chromatography eluted with 10% to 25% EtOAc in petroleum ether to give the title compound (1.2 g, 69%) as a white solid. MS ES+ m/z 327, 329 [M+H]⁺.

### Intermediate 59A: 2-Benzylsulfanyl-3-bromo-6-chloro-pyridine

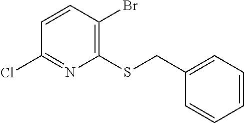 [SMILES: Clc1ccc(Br)c(SCc2ccccc2)n1]

A solution of phenylmethanethiol (0.97 g, 7.80 mmol) in THF (20 mL) was cooled to 0° C. and treated with sodium hydride (0.31 g, 7.80 mmol). After stirring at 0° C., the reaction was treated with 3-bromo-6-chloro-2-fluoropyridine (1.5 g, 7.10 mmol). The reaction was allowed to stir at rt for 1.5 h. The reaction was concentrated and purified by silica gel chromatography eluted with 0% to 100% EtOAc in heptane to give the title compound (2.10 g, 94%). MS ES+ m/z 314 [M+H]⁺.

### Intermediate 60A: 8-Bromo-2-[(4-methoxyphenyl)methyl]-3,4-dihydroisoquinolin-1-one

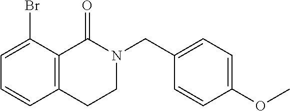 [SMILES: COc1ccc(CN2CCc3cccc(Br)c3C2=O)cc1]

Dissolved 8-bromo-3,4-dihydro-2H-isoquinolin-1-one (200 mg, 0.89 mmol) in 5 mL of THF and cooled to 0° C. When cold, treated the solution with sodium hydride (38.9 mg, 60%, 0.97 mmol). After stirring at 0° C. for 20 min, treated the reaction with 4-methoxybenzyl chloride (152 mg, 0.97 mmol) and allowed to stir at rt for 2 h. Added an additional 152 mg of 4-methoxybenzyl chloride and stirred at 50° C. for 23 h. The reaction was partitioned between water and EtOAc. The organic layer was removed and the aqueous layer extracted with EtOAc (2×). The organic layers were combined, washed with saturated aqueous sodium chloride, and dry loaded onto celite. Purified by silica gel chromatography eluted with 0% to 40% EtOAc in heptane to give the title compound (355 mg, >100%) as a colorless oil containing some 4-methoxybenzyl chloride. MS ES+ m/z 347 [M+H]⁺.

### Intermediate 61A: 8-Bromo-2-(2-trimethylsilylethoxymethyl)isoquinolin-1-one

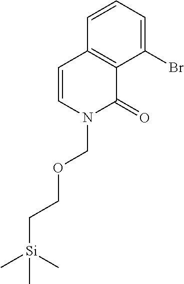 [SMILES: C[Si](C)(C)CCOCn1ccc2cccc(Br)c2c1=O]

A 20 mL vial was charged with 8-bromo-2H-isoquinolin-1-one (0.34 g, 1.53 mmol) and DMF (5 mL), treated with sodium hydride (73.5 mg, 60%, 1.84 mmol), and stirred at rt for 10 min. The reaction was treated with 2-(trimethylsilyl)ethoxymethyl chloride (0.51 g, 3.06 mmol) and stirred at rt for 1 h. The reaction was treated with another 0.51 g of 2-(trimethylsilyl)ethoxymethyl chloride and the reaction stirred at 50° C. over the weekend. The reaction was partitioned between water and EtOAc. The organic layer was collected and the aqueous layer extracted with EtOAc (2×). The organic layers were combined, washed with saturated aqueous sodium chloride, and concentrated. The residue was purified by silica gel chromatography eluted with 0% to 40% EtOAc in heptane to give the title compound (231 mg, 432%) as an opaque oil. MS ES+ m/z 354/356 [M+H]⁺.

### Intermediate 62A: (2-Bromo-4-methyl-phenyl) acetate

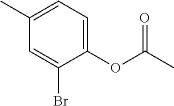

A DCM (2.4 L) mixture of 2-bromo-4-methyl-phenol (300 g, 1.6 mol) and pyridine (152 g, 1.92 mol) at 0° C. was treated with acetyl chloride and stirred at 25° C. for 16 h. The mixture was diluted with water (1500 mL), the pH adjusted to 5 with HCl (2 M aqueous), and extracted with DCM (3×500 mL). The combined organic extracts were washed with saturated aqueous sodium chloride (2×250 mL), dried over Na₂SO₄, filtered, and concentrated to give the product as an oil (400 g, crude). ¹H NMR (400 MHz, CDCl₃) δ ppm 2.24 (s, 3H), 2.25 (s, 3H), 6.91 (d, J=8.4 Hz, 2H), 7.01-7.02 (m, 2H), 7.33 (s, 1H).

### Intermediate 63A: 1-(3-Bromo-2-hydroxy-5-methyl-phenyl)ethenone

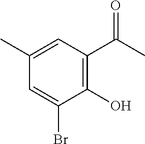

A mixture of (2-bromo-4-methyl-phenyl) acetate (50 g, 218 mmol) and AlCl₃ (102 g, 764 mmol) was degassed and purged with N2 three times and stirred at 140° C. for 1 h. After cooling to rt, the reaction was diluted with DCM (30 mL) and dropped into 150 mL of water at 0° C. The mixture was filtered and the aqueous phase extracted with DCM (2×150 mL). The combined organic extracts were washed with saturated aqueous sodium chloride, dried over anhydrous Na₂SO₄, filtered, and concentrated. The residue was triturated with petroleum ether (2×150 mL) to give the product as a solid (30 g, 52%). ¹H NMR (400 MHz, CDCl₃) δ ppm 2.30 (s, 3H), 2.68 (s, 3H), 7.73 (s, 1H), 7.33 (s, 1H), 12.64 (s, 1H).

### Intermediate 2A: 1-(3-Bromo-2-hydroxy-5-methyl-phenyl)propan-1-one

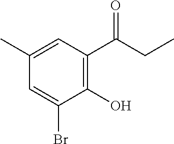

A mixture of (2-bromo-4-methyl-phenyl) propanoate (12.5 g, 51.4 mmol) and AlCl₃ (24.0 g, 180 mmol) was stirred at 140° C. for 1 h. When cooled to rt, the mixture was quenched with water (80 mL) dropwise and stirred for 30 min. The mixture was extracted with EtOAc (3×100 mL). The combined organic extracts were washed with saturated aqueous sodium chloride (2×200 mL), dried over anhydrous Na₂SO₄, and filtered. The filtrate was concentrated and triturated with petroleum ether (20 mL) to give the product as a solid (9.82 g, 79%). ¹H NMR (400 MHz, DMSO-d6) δ ppm 1.10 (t, J=7.2 Hz, 3H), 2.28 (s, 3H), 3.15 (q, J=7.2 Hz, 2H), 7.66-7.73 (m, 1H), 7.77-7.83 (m, 1H), 12.66 (s, 1H).

### Intermediate 30C: 1-(3-Bromo-5-fluoro-2-hydroxy-phenyl)propan-1-one

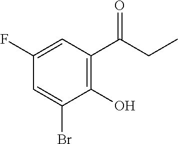

(2-Bromo-4-fluoro-phenyl) propanoate (3.5 kg, 14.2 mol) in a reactor was heated to 75˜85° C. and treated with AlCl₃ (1.0 kg, 7.4 mol) in batches while the internal temperature increased to 100˜110° C. The reaction was heated to 100˜120° C. and treated with more AlCl₃ (1.1. kg, 8.2 mol). The reaction was stirred at 110˜130° C. for 18 h. The reaction was cooled to rt and poured into a mixture of MTBE (20 L) and 1M aqueous HCl (20 L) at 0˜10° C. The reaction was stirred at 0˜15° C. for 1 h. The organic layer was separated and washed with water (2×10 L). The organic phase was dried over MgSO₄ and concentrated under reduced pressure to afford a crude product. The crude material was slurried with n-heptane (7 L) at rt for 6 h and then filtered to give the title compound (2.0 kg, 57%) as a brown solid after drying at 45° C. for 12 h. H NMR (400 MHz, DMSO-d6) δ ppm 1.15 (t, 3H), 3.20 (m, 2H), 7.95 (m, 2H), 12.50 (s, 1H).

### Intermediate 31C: 1-(3-Bromo-2-hydroxy-5-methyl-phenyl)propan-1-one

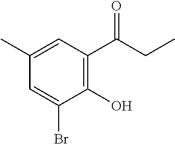

Trifluoromethanesulfonic acid (237 g, 1.58 mol) was cooled to below −5° C. and treated dropwise with (2-bromo-4-methyl-phenyl) propanoate (80 g, 329 mmol) over 1 h keeping the temperature between −5° C. and −20° C. After addition was completed, the cooling bath was removed and the reaction heated at 55-60° C. for 1.5 h. The mixture was cooled to 5-10° C., poured into ice water (5V), stirred for 30 min, and the solid removed by filtration. The cake was rinsed with water (2V), slurried in water (5V) for 30 min, and filtered to give the title compound (72.2 g, 90%) as an off-white solid after drying. ES/MS m/z 241/243 (M−H).

### Intermediate 64A: 1-[2-Methoxy-5-(trifluoromethyl)phenyl]propan-1-one

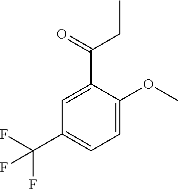 [SMILES: CCC(=O)c1cc(C(F)(F)F)ccc1OC]

A 10 L reactor was charged with trifluoromethanesulfonic acid (5.54 kg, 36.90 mol) and cooled to −10° C. to −20° C. When cold, propionyl chloride (2.05 kg, 22.14 mol) was slowly added and stirred for 0.5 h. 1-Methoxy-4-(trifluoromethyl)benzene (1.30 kg, 7.38 mol) was slowly added for 2 h keeping the temperature between −10° C. and −20° C. The resulting solution was stirred for 20 h. The solution was slowly added to ice cold water (13 L) keeping the temperature at 0° C. to 10° C. and then stirred for 1 h. The suspension was then filtered and the solids rinsed with ice-cold water (7.5 L). The cake was dissolved in MTBE (3.9 L) and the aqueous phase discarded. The organic phase was concentrated to give the title compound (1.65 kg, 96%) as an off-white solid. ¹H NMR (400 MHz, CDCl₃) δ ppm 1.13-1.25 (3H, t, J=7.24 Hz), 2.95-3.08 (2H, q, J=7.23 Hz), 3.97 (3H, s), 7.06 (1H, d, J=8.68 Hz), 7.60-7.73 (1H, m), 7.95 (1H, d, J=2.16 Hz).

### Intermediate 65A: 1-[2-Hydroxy-5-(trifluoromethyl)phenyl]propan-1-one

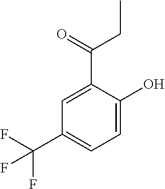

A mixture of NaOH (413.4 g, 10.34 mol) in DMF (8.25 L) was treated with 1-dodecanethiol (2.09 kg, 10.34 mol) at 25° C. and the mixture stirred at 25° C. for 1 h. The reaction was treated with 1-[2-methoxy-5-(trifluoromethyl)phenyl]propan-1-one (1.60 kg, 6.89 mol) and the reaction heated to 70° C. and stirred at that temperature for 1.5 h. The mixture was diluted with water (24 L) and extracted with MTBE (16 L). The pH of the aqueous phase was adjusted to 2-3 with aqueous HCl (2 N) and extracted with MTBE (16 L). The organic phase was washed with 15% aqueous NaCl (8 L), collected, and concentrated to give the title compound (1.24 kg, 82%) as a yellow oil. ¹H NMR (400 MHz, CDCl₃) δ ppm 1.25-1.32 (3H, t, J=7.22 Hz), 3.02-3.17 (2H, q, J=7.21 Hz), 3.97 (3H, s), 7.05-7.13 (1H, d, J=8.76 Hz), 7.65-7.74 (1H, m), 8.04 (1H, d, J=1.28 Hz), 12.66 (1H, s).

### Intermediate 66A: 1-[3-Bromo-2-hydroxy-5-(trifluoromethyl)phenyl]propan-1-one

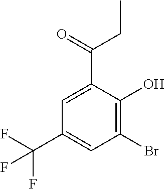 [SMILES: CCC(=O)c1cc(C(F)(F)F)cc(Br)c1O]

A mixture of 1-[2-hydroxy-5-(trifluoromethyl)phenyl]propan-1-one (1.20 kg, 5.50 mol) in DMF (4.8 L) was treated with N-bromosuccinimide (1.08 kg, 6.05 mol) at 5° C. to 15° C. and the mixture stirred at 20° C. to 30° C. for 5 h. The reaction was diluted with water (14.4 L) and saturated aqueous Na₂SO₃ (3.6 L) and stirred at 25° C. for 1 h. The mixture was filtered and the cake slurried in water (6 L) at 20° C. to 25° C. for 0.5 h, filtered, rinsed with water (3.6 L), and dried at 50° C. to afford the title compound (1.38 kg, 84%) as an off-white solid. ¹H NMR (400 MHz, DMSO-d6) δ ppm 1.08-1.14 (3H, t, J=7.04 Hz), 3.23-3.31 (2H, q, J=7.05 Hz), 3.97 (3H, s), 8.21-8.35 (2H, d, J=8.80 Hz), 13.21 (1H, s).

### Intermediate 32C: 1-(3-Bromo-2-hydroxy-5-methyl-phenyl)-2-methyl-3-(3-pyridyl)propane-1,3-dione

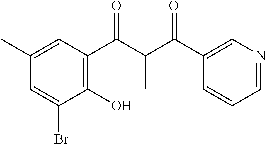 [SMILES: Cc1cc(Br)c(O)c(C(=O)C(C)C(=O)c2cccnc2)c1]

A suspension of 1-(3-bromo-2-hydroxy-5-methyl-phenyl)propan-1-one (300 g, 1.23 mol) in THF (600 mL) was cooled to −70˜−75° C. and treated with lithium bis(trimethylsilyl)amide (5.4 L, 1M in THF, 5.4 mol) over 14 h maintaining the internal temperature below −60° C. After addition was complete, stirred at ˜60 to −75° C. for 30 min. The reaction was cooled to −70 to ˜75° C. and treated with pyridine-3-carbonyl chloride hydrochloride (285.6 g, 1.60 mol) portionwise maintaining the internal temperature below −60° C. After addition, the reaction was stirred at rt for 15 h. The reaction was cooled to 0 to −10° C. and quenched with AcOH/water (3V/3V) until the pH reached 5-6. The reaction was concentrated under reduced pressure and the title compound (600 g, 100%) removed by filtration, rinsed with water, and air dried. ES/MS m/z 348/350 (M+H).

### Intermediate 67A: 8-Bromo-4-hydroxy-6-methyl-chromene-2-thione

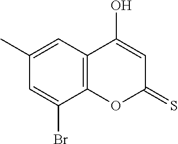

A solution of 1-(3-bromo-2-hydroxy-5-methyl-phenyl)ethanone (65 g, 284 mmol) in THF (800 mL) was treated with NaHMDS (851 mL, 1 M) at −50° C. over 30 min, allowed to warm to between −5° C. and 0° C., and stirred for 1 h. The reaction was cooled to −20° C. and treated with CS₂ (64.8 g, 851 mmol) dropwise over 1 h, allowed to warm to 25° C., and stirred for another 16 h. The reaction was quenched with H₂SO₄ (800 mL, 15%) at −50° C. over 1 h, allowed to warm to rt, and extracted with EtOAc (2×1L). The combined organic extracts were washed with saturated aqueous sodium chloride (1L), dried over anhydrous Na₂SO₄, filtered, and concentrated. The residue was triturated with EtOAc (0.5 L) to give the product as a solid (210 g crude, 64%, purity ˜76%).

### Intermediate 33C: 8-Bromo-6-fluoro-4-hydroxy-3-methyl-chromene-2-thione

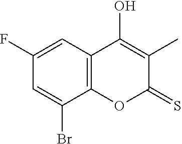

Potassium tert-butoxide (3.2 kg, 28.3 mol) and THF (30 L) were added to a 50 L reactor and the mixture stirred for 15 min to give a light yellow solution. Added 8-bromo-6-fluoro-4-hydroxy-3-methyl-chromene-2-thione (2.0 kg, 8.1 mol) in batches to the reactor keeping the temperature between 0 and 15° C. Carbon disulfide (740 g, 9.7 mol) was added dropwise keeping the temperature below 30° C. After addition was completed, the reaction was stirred at 0 to 25° C. for 12 h. The reaction mixture was poured directly into 15% aqueous acetic acid (5 L) and stirred at rt for 30 min. The mixture was filtered and the filter cake washed with MTBE (2 L). The filtrate was washed with water (2×5 L) and saturated aqueous NaCl (5 L). The organic phase was collected, dried over MgSO₄, filtered, and concentrated under reduced pressure to afford the crude product. This material was slurried in heptane (5 L), stirred for 6 h, and filtered to give the title compound (2.8 kg, 120%) as a yellow solid after drying at 45° C. for 12 h. ¹H NMR (400 MHz, DMSO-d6) δ ppm 2.10 (s, 3H), 7.65 (m, 1H), 7.90 (m, 1H).

The following compound in Table 4 was made in a similar way as described for 8-bromo-6-fluoro-4-hydroxy-3-methyl-chromene-2-thione. Various methods were used to purify this compound, which would be apparent to one skilled in the art.

TABLE 4

| | | | MS ES+ |
|---|---|---|---|
| Interm # | Chemical Name | Structure | m/z |
| 34C | 8-Bromo-6-fluoro-4-hydroxy- chromene-2-thione | 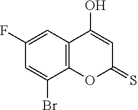 [SMILES: Oc1cc(=S)oc2c(Br)cc(F)cc12] | |

### Intermediate 3A: (E)-1-(3-Bromo-2-hydroxy-5-methyl-phenyl)-2-methyl-3-phenyl-prop-2-en-1-one

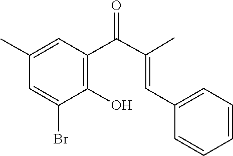 [SMILES: C/C(=C\c1ccccc1)C(=O)c1cc(C)cc(Br)c1O]

A mixture of 1-(3-bromo-2-hydroxy-5-methyl-phenyl)propan-1-one (200 g, 822.72 mmol), benzaldehyde (96.04 g, 904.99 mmol), AcOH (105.23 g, 1.75 mol), and piperidine (172.33 g, 2.02 mol) in EtOH (1600 mL) was stirred at 70° C. for 16 h. The resulting dark solution was poured into water (3 L), filtered, and the solid dissolved in 6 L of DCM. The organic solution was dried over anhydrous Na₂SO₄, filtered, and concentrated to give the product as a dark gum. MS ES+ m/z 331, 333 [M+H]⁺.

### Intermediate 68A: 8-Bromo-2-ethylsulfanyl-6-methyl-chromen-4-one

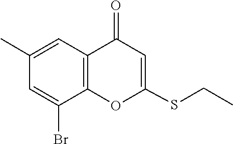 [SMILES: CCSc1cc(=O)c2cc(C)cc(Br)c2o1]

A mixture of 8-bromo-4-hydroxy-6-methyl-chromene-2-thione (20.0 g, 73.8 mmol), EtI (46 g, 295 mmol), and K2CO₃ (12.2 g, 88.5 mmol) in acetone (200 mL) was stirred at 60° C. for 3 h. When the reaction had cooled to rt, the mixture was diluted with water (200 mL) and extracted with DCM (2×200 mL). The combined organic extracts were concentrated and purified by silica gel chromatography eluted with 20% to 40% EtOAc in petroleum ether to give the product as a gum. ¹H NMR (400 MHz, CDCl₃) δ ppm 1.51 (t, J=7.2 Hz, 3H), 2.45 (s, 3H), 3.22 (q, J=7.2 Hz, 2H), 6.32 (s, 1H), 7.70 (s, 1H), 7.93 (s, 1H).

The following compound in Table 5 was made in a similar way as described for 8-bromo-2-ethylsulfanyl-6-methyl-chromen-4-one. Various methods were used to purify this compound, which would be apparent to one skilled in the art.

TABLE 5

| | | | MS ES+ |
|---|---|---|---|
| Interm # | Chemical Name | Structure | m/z |
| 35C | 8-Bromo-2-ethylsulfanyl-6-fluoro-3- methyl-chromen-4-one | 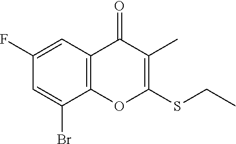 [SMILES: CCSc1oc2c(Br)cc(F)cc2c(=O)c1C] | |

### Intermediate 36C: 8-Bromo-2-ethylsulfanyl-6-fluoro-chromen-4-one

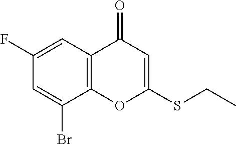 [SMILES: CCSc1cc(=O)c2cc(F)cc(Br)c2o1]

The crude THF solution from the synthesis of 8-bromo-6-fluoro-4-hydroxy-chromene-2-thione (3.4 kg, 30 mol) was treated with EtI (5.4 kg, 34.4 mol) at 20 to 25° C. The resulting solution was stirred at 60 to 65° C. for 12 h. The reaction was cooled to rt and a solid removed by filtration. The cake was washed with MTBE (2 L). The filtrates were combined, washed with water (2×6 L), and washed with 10% aqueous NaCl. The organic phase was collected and concentrated under reduced pressure to afford the crude product. The crude solid was slurried with heptane (12 L) and stirred at rt for 3 h. Removed the title compound (2.0 kg, 77%) as a yellow solid. ¹H NMR (400 MHz, DMSO-d6) δ ppm 1.40 (t, 3H), 3.26 (dd, 2H), 6.47 (s, 1H), 7.69 (m, 1H), 8.15 (m, 1H).

### Intermediate 69A: 8-Bromo-2-ethylsulfanyl-3-methyl-6-(trifluoromethyl)chromen-4-one

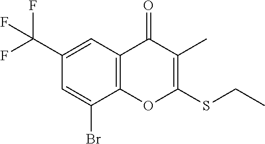 [SMILES: CCSc1oc2c(Br)cc(C(F)(F)F)cc2c(=O)c1C]

A mixture of 1-[3-bromo-2-hydroxy-5-(trifluoromethyl)phenyl]propan-1-one (1.26 kg, 4.24 mol) in THF (3.0 L) was slowly treated with lithium tert-butoxide (1.36 kg, 16.97 mol) and stirred for 0.5 h. The reaction was then treated with carbon disulfide (355.2 g, 4.67 mol) in one portion and the mixture stirred at 15° C. to 25° C. for 16 h. The reaction was added to 15% aqueous H₂SO₄ (12.6 L) at 0° C. to 10° C. The organic layer was removed and the aqueous layer extracted with 2-methyltetrahydrofuran (12.6 L). The organic layers were combined, washed with saturated aqueous sodium chloride (6.3 L), and concentrated to give a crude yellow solid. The solid was slurried with DCM (3.6 L) for 1 h and filtered. The cake was dried under vacuum at 40° C. to afford 8-bromo-3-methyl-2-sulfanyl-6-(trifluoromethyl)chromen-4-one as a yellow solid.

A 10 L reactor was charged with 8-bromo-3-methyl-2-sulfanyl-6-(trifluoromethyl)chromen-4-one (from above), cesium carbonate (586.6 g, 1.8 mol), and acetone (6.3 L). The reaction was slowly treated with EtI (421.1 g, 2.7 mol) at 15° C. to 25° C. and then allowed to stir for 2 h. The reaction was filtered and rinsed with acetone (3.78 L). The filtrate was concentrated to afford the title compound (650 g, 42%) as a yellow solid. ¹H NMR (400 MHz, DMSO-d6) δ ppm 1.40-1.48 (3H, t, J=7.30 Hz), 1.93 (3H, s), 3.33-3.40 (2H, q, J=7.32 Hz), 8.18 (1H, d, J=1.52 Hz), 8.45 (1H, d, J=2.00 Hz).

### Intermediate 4A: 8-Bromo-3,6-dimethyl-2-phenyl-chromen-4-one

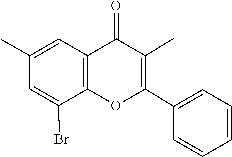 [SMILES: Cc1cc(Br)c2oc(-c3ccccc3)c(C)c(=O)c2c1]

A mixture of (E)-1-(3-bromo-2-hydroxy-5-methyl-phenyl)-2-methyl-3-phenyl-prop-2-en-1-one (284 g, 857.48 mmol) and I₂ (21.76 g, 85.75 mmol) in DMSO (1200 mL) was stirred at 140° C. for 2 h to give a black-brown solution. Cooled to rt, poured the reaction into 3 L of water, filtered, dissolved the solid product in DCM (4 L), dried over anhydrous Na₂SO₄, filtered, and concentrated to give a residue. The residue was triturated with petroleum ether/EtOAc (1:1, 1 L) to give the product as a light yellow solid (195 g, 69%). MS ES+ m/z 329, 331 [M+H]⁺.

### Intermediate 37C: 8-Bromo-3,6-dimethyl-2-(3-pyridyl)chromen-4-one

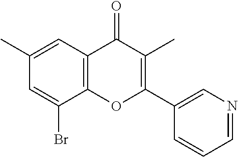 [SMILES: Cc1cc(Br)c2oc(-c3cccnc3)c(C)c(=O)c2c1]

Crude 1-(3-bromo-2-hydroxy-5-methyl-phenyl)-2-methyl-3-(3-pyridyl)propane-1,3-dione (600 g, 1.23 mol) was suspended in HCl (0.15 V) and AcOH (3 V) and stirred at 90 to 100° C. for 2 h. The reaction was cooled to rt and basified to pH 8-9 with 5M aqueous NaOH keeping the temperature below 50° C. The resulting precipitate was collected by filtration and rinsed with water (2×2V). The solid was slurried with MTBE (5V), stirred at rt for 40 h, and the title compound collected by filtration (368 g, 90%) after drying at 50° C. ES/MS m/z (⁷⁹Br/⁸¹Br) 330/332 (M+H).

### Intermediate 70A: 8-Acetyl-2-ethylsulfanyl-6-methyl-chromen-4-one

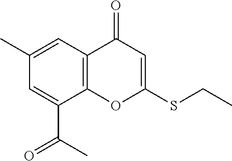 [SMILES: CCSc1cc(=O)c2cc(C)cc(C(C)=O)c2o1]

A mixture of 8-bromo-2-ethylsulfanyl-6-methyl-chromen-4-one (9.00 g, 30.0 mmol), tributyl(1-ethoxyvinyl)tin (13.3 g, 36.8 mmol) and Pd(PPh₃)₂Cl₂ (2.11 g, 3.01 mmol) in 1,4-dioxane (90 mL) was stirred at 95° C. for 16 h. HCl (30 mL, 1 M) was added to the mixture and stirred at 50° C. for 0.5 h. When cooled to rt, the mixture was treated with saturated aqueous KF (100 mL) and stirred for 0.5 h, then filtered. The filter cake was washed with EtOAc (3×40 mL). The filtrate was extracted with EtOAc (2×80 mL). The combined organic extracts were concentrated and purified by silica gel chromatography eluted with 0% to 60% EtOAc in petroleum ether to give the product as a solid (5.8 g, 60%). MS ES+ m/z 263 [M+H]⁺.

### Intermediate 5A: 8-Acetyl-3,6-dimethyl-2-phenyl-chromen-4-one

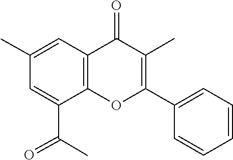 [SMILES: CC(=O)c1cc(C)cc2c(=O)c(C)c(-c3ccccc3)oc12]

A mixture of 8-bromo-3,6-dimethyl-2-phenyl-chromen-4-one (195 g, 592.37 mmol), bis(triphenylphosphine)palladium(II) dichloride (20.79 g, 29.62 mmol), and tributyl(1-ethoxyvinyl)stannane (256.72 g, 710.84 mmol, 239.92 mL) in dioxane (1600 mL) was stirred under N2 at 95° C. for 16 h to give a black-brown solution. After cooling to rt, treated the reaction with 1M aqueous HCl (100 mL) and stirred at 20° C. for 30 min. The mixture was quenched with saturated aqueous KF (2000 mL), stirred for 30 min, and filtered. The filter cake was washed with 10% MeOH in DCM (5×5000 mL). The combined extracts were dried over anhydrous Na₂SO₄, filtered, and concentrated to give a residue. The residue was triturated with petroleum ether/EtOAc (5/1, 1000 mL) to give a crude product which was triturated with DCM/MeOH (10/1, 500 mL) to give the product as a light yellow solid (180 g, 96%, 92% purity). MS ES+ m/z 293 [M+H]⁺.

### Intermediate 71A: 8-Acetyl-2-ethylsulfanyl-3-methyl-6-(trifluoromethyl)chromen-4-one

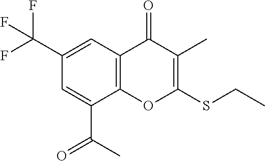 [SMILES: CCSc1oc2c(C(C)=O)cc(C(F)(F)F)cc2c(=O)c1C]

A mixture of 8-bromo-2-ethylsulfanyl-3-methyl-6-(trifluoromethyl)chromen-4-one (980 g, 2.67 mol) in 1,4-dioxane (3.3 L) was treated with tributyl(1-ethoxyvinyl)stannane (1.06 kg, 2.94 mol). The reaction was evacuated and nitrogen used to replace the air three times and then bis(triphenylphosphine)palladium(II) dichloride (74.93 g, 106.8 mmol) was added in one portion. The reaction was evacuated and the nitrogen used to replace the air three more times and the reaction heated to 85° C. to 95° C. for 14 h. The reaction was allowed to cool to 50° C. and then aqueous HCl (4.9 L, 2 M) was added and the reaction stirred for 2 h. The reaction was cooled to 20° C. to 25° C. and then extracted with EtOAc (14.7 L). The organic phase was quenched with saturated aqueous KF (4.9 L), stirred for 1 h, and filtered. The organic phase was washed with saturated aqueous sodium chloride (4.9 L) and the organic phase concentrated to 4.9 L under reduced pressure. IPA (9.8 L) was added and the mixture concentrated to 4.9 L two more times. IPA (4.9 L) was added to the mixture and heated to 70° C. The mixture was filtered, the filtrate cooled to 0° C., stirred for 14 h, filtered, and the solids rinsed with TPA (1.96 L). Repeated the above crystallization process once again and the combined solids dried in the vacuum at 40° C. to afford the title compound (455.7 g, 520) as an off-white solid. MS ES+ m/z 331 [M+H]⁺.

The following compounds in Table 6 were made in a similar way as described for 8-acetyl-2-ethylsulfanyl-3-methyl-6-(trifluoromethyl)chromen-4-one. Various methods were used to purify the compounds, which would be apparent to one skilled in the art.

TABLE 6

| | | | MS ES+ |
|---|---|---|---|
| Interm # | Chemical Name | Structure | m/z |
| 38C | 8-Acetyl-3,6-dimethyl-2-(3- pyridyl)chromen-4-one | 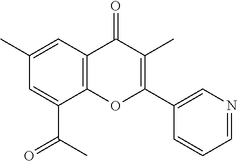 [SMILES: CC(=O)c1cc(C)cc2c(=O)c(C)c(-c3cccnc3)oc12] | 294 [M + H]⁺ |
| 39C | 8-Acetyl-2-ethylsulfanyl-6-fluoro-3- methyl-chromen-4-one | 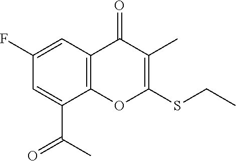 [SMILES: CCSc1oc2c(C(C)=O)cc(F)cc2c(=O)c1C] | 281 [M + H]⁺ |
| 40C | 8-Acetyl-2-ethylsulfanyl-6-fluoro- chromen-4-one | 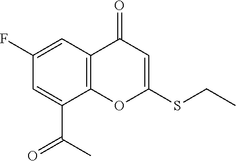 [SMILES: CCSc1cc(=O)c2cc(F)cc(C(C)=O)c2o1] | 267 [M + H]⁺ |

### Intermediate 72A: 2-Ethylsulfanyl-8-(1-hydroxyethyl)-6-methyl-chromen-4-one

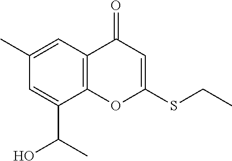 [SMILES: CCSc1cc(=O)c2cc(C)cc(C(C)O)c2o1]

A solution of 8-acetyl-2-ethylsulfanyl-6-methyl-chromen-4-one (8.30 g, 31.6 mmol) in DCM (30 mL) and MeOH (30 mL) was treated with NaBH₄ (1.32 g, 34.8 mmol) in portions at 0° C., and stirred at 15° C. for 1 h. The mixture was diluted with water (50 mL) and extracted with DCM (2×100 mL). The combined organic extracts were washed with saturated aqueous sodium chloride (80 mL), dried over anhydrous Na₂SO₄, filtered, and concentrated. The residue was purified by silica gel chromatography eluted with 0% to 4% MeOH in DCM to give the product as a solid (6.0 g, 60%). MS ES+ m/z 265 [M+H]⁺.

The following compound in Table 7 was made in a similar way as described for 2-ethylsulfanyl-8-(1-hydroxyethyl)-6-methyl-chromen-4-one. Various methods were used to purify the compound, which would be apparent to one skilled in the art.

TABLE 7

| Interm # | Chemical Name | Structure | Physical data |
|---|---|---|---|
| 41C | 2-Ethylsulfanyl-6- fluoro-8-(1- hydroxyethyl)-3- methyl-chromen-4- one | 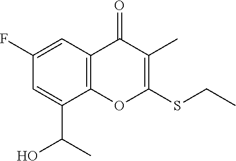 [SMILES: CCSc1oc2c(C(C)O)cc(F)cc2c(=O)c1C] | ¹H NMR (400 MHz, DMSO-d6) δ ppm: 1.47~1.51 (m, 3H), 1.56~1.58 (m, 6H), 2.06 (s, 3H), 2.63 (m, 1H) 3.19~3.25 (m, 2H), 5.39~5.44 (m, 1H), 5.62~5.63 (m, 1H), 7.50~7.53 (m, 1H), 7.61~7.64 (m, 1H) |
| 42C | 2-Ethylsulfanyl-6- fluoro-8-(1- hydroxyethyl)chromen- 4-one | 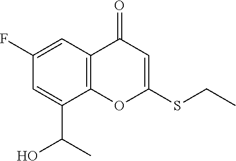 [SMILES: CCSc1cc(=O)c2cc(F)cc(C(C)O)c2o1] | ¹HNMR (400 MHz, CDCl₃) δ ppm 1.46~1.50 (m, 3H), 1.58~1.60 (m, 3H), 2.23~2.24 (m, 1H), 3.08~3.13 (m, 2H), 5.41~5.47 (m, 1H), 6.26 (s, 1H), 7.58~7.61 (m, 1H), 7.69~7.71 (m, 1H) |

### Intermediate 43C: 2-Ethylsulfanyl-8-[(1S)-1-hydroxyethyl]-3-methyl-6-(trifluoromethyl)chromen-4-one

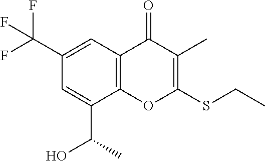 [SMILES: CCSc1oc2c([C@H](C)O)cc(C(F)(F)F)cc2c(=O)c1C]

A solution of 8-acetyl-2-ethylsulfanyl-3-methyl-6-(trifluoromethyl)chromen-4-one (140 g, 0.42 mol) in DCM (1.4 L) was cooled to −5° C. under a nitrogen atmosphere. Formic acid (58.5 g, 1.27 mol) was added slowly followed by dropwise addition of triethylamine (86.4 g, 0.85 mol) maintaining the temperature below 10° C. The Noyori catalyst (CAS: 74813-81-1, 2.7 g, 4.24 mmol) was added in one portion and the reaction allowed to warm to rt and stir for 18 h. The reaction was washed with 1 M aqueous HCl (2×500 mL), water (200 mL), and saturated aqueous NaCl (100 mL). The organic layer was collected, dried over MgSO₄, filtered, and concentrated under reduced pressure to give the title compound (146 g, 100%, 99% purity) as a brown solid with a 93:7 enantiomeric ratio. ES/MS m/z 333 (M+H).

### Intermediate 73A: 8-(1-Bromoethyl)-2-ethylsulfanyl-6-methyl-chromen-4-one

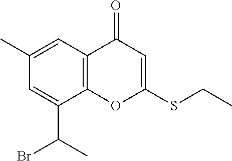 [SMILES: CCSc1cc(=O)c2cc(C)cc(C(C)Br)c2o1]

A mixture of 2-ethylsulfanyl-8-(1-hydroxyethyl)-6-methyl-chromen-4-one (5.50 g, 20.8 mmol) in DCM (50 mL) was treated dropwise with PBr₃ (16.9 g, 62.4 mmol) at 0° C., then stirred at 30° C. for 4 h. The reaction was quenched with water (20 mL) at 0° C. and the pH adjusted to 8 with saturated aqueous NaHCO₃. The mixture was extracted with DCM (2×80 mL). The combined organic extracts were washed with saturated aqueous sodium chloride, dried over anhydrous Na₂SO₄, filtered, and concentrated to give the product as an oil (4.7 g, 61%). MS ES+ m/z 329 [M+2+H]⁺.

The following compounds in Table 8 were made in a similar way as described for 8-(1-bromoethyl)-2-ethylsulfanyl-6-methyl-chromen-4-one. Various methods were used to purify the compounds, which would be apparent to one skilled in the art.

TABLE 8

| | | | MS ES+ |
|---|---|---|---|
| Interm # | Chemical Name | Structure | m/z |
| 44C¹ | 8-(1-Bromoethyl)-2-ethylsulfanyl-6- fluoro-3-methyl-chromen-4-one | 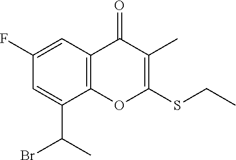 [SMILES: CCSc1oc2c(C(C)Br)cc(F)cc2c(=O)c1C] | |
| 45C | 8-[(1R)-1-Bromoethyl]-2- ethylsulfanyl-3-methyl-6- (trifluoromethyl)chromen-4-one | 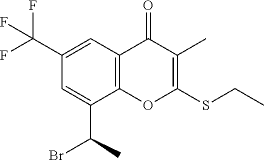 [SMILES: CCSc1oc2c([C@@H](C)Br)cc(C(F)(F)F)cc2c(=O)c1C] | 395/397 [M + H]⁺ |
| 46C² | 8-(1-Bromoethyl)-2-ethylsulfanyl-6- fluoro-chromen-4-one | 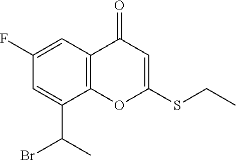 [SMILES: CCSc1cc(=O)c2cc(F)cc(C(C)Br)c2o1] | |

¹¹HNMR (400 MHz, CDCl₃) δ ppm: 1.50~1.54 (m, 3H), 2.10 (s, 3H), 2.15~2.17 (m, 3H), 3.27~3.42 (m, 2H), 5.64~5.69(m, 1H), 7.50~7.53 (m, 1H), 7.82~7.84 (m, 1H).
²¹HNMR (400 MHz, CDCl₃) δ ppm 1.48~1.51 (m, 3H), 2.13~2.15 (m, 3H), 3.12~3.27 (m, 2H), 5.64~5.69 (m, 1H), 6.32 (s, 1H), 7.55~7.57 (m, 1H), 7.79~7.81 (m, 1H).

### Intermediate 74A: (NE,R)—N-[1-(2-Ethylsulfanyl-6-methyl-4-oxo-chromen-8-yl)ethylidene]-2-methyl-propane-2-sulfinamide

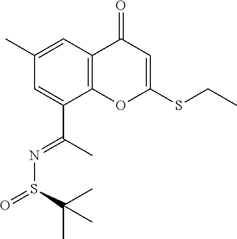 [SMILES: CCSc1cc(=O)c2cc(C)cc(/C(C)=N/[S@](=O)C(C)(C)C)c2o1]

A mixture of 8-acetyl-2-ethylsulfanyl-6-methyl-chromen-4-one (9.49 g, 36.2 mmol) and (R)-2-methylpropane-2-sulfinamide (8.77 g, 72.4 mmol) in THF (100 mL) was treated with Ti(i-PrO)₄ (41.1 g, 145 mmol) and stirred at 75° C. for 16 h. The reaction was treated with additional (R)-2-methylpropane-2-sulfinamide (6.58 g, 54.3 mmol) and Ti(i-PrO)₄ (30.9 g, 109 mmol) and stirred at 75° C. for another 16 h. The mixture was quenched with saturated aqueous sodium chloride (200 mL), stirred for 0.5 h, and filtered. The filter cake was washed with EtOAc (300 mL). The aqueous layer was extracted with EtOAc (2×300 mL). The combined organic extracts were washed with saturated aqueous sodium chloride (2×200 mL), dried over anhydrous Na₂SO₄, filtered, and concentrated to give the product as a solid (13 g, crude). MS ES+ m/z 366 [M+H]⁺.

### Intermediate 6A: (NE,R)—N-[1-(3,6-Dimethyl-4-oxo-2-phenyl-chromen-8-yl)ethylidene]-2-methyl-propane-2-sulfinamide

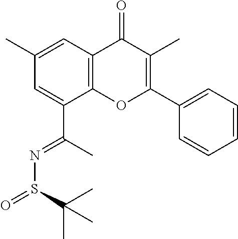 [SMILES: C/C(=N\[S@](=O)C(C)(C)C)c1cc(C)cc2c(=O)c(C)c(-c3ccccc3)oc12]

To a mixture of 8-acetyl-3,6-dimethyl-2-phenyl-chromen-4-one (180 g, 615.75 mmol) and (R)-2-methylpropane-2-sulfinamide (149.26 g, 1.23 mol) in THF (1500 mL) was added tetraisopropoxytitanium (700.01 g, 2.46 mol, 726.90 mL). The mixture was stirred at 80° C. for 56 h to give a black-brown solution. After cooling to rt, quenched the reaction with saturated aqueous sodium chloride (2000 mL) and stirred for 30 min and filtered. The filter cake was washed with EtOAc (4000 mL). After separating the organic layer, the aqueous layer was extracted with EtOAc (1000 mL). The combined organic extracts were dried over anhydrous Na₂SO₄, filtered, and concentrated to give a residue. The residue was triturated with petroleum ether/EtOAc (1/1, 600 mL) to give the product as a white solid (186 g, 76%). MS ES+ m/z 396 [M+H]⁺.

The following compound in Table 9 was made in a similar way as described for (NE,R)—N-[1-(3,6-dimethyl-4-oxo-2-phenyl-chromen-8-yl)ethylidene]-2-methyl-propane-2-sulfinamide.

TABLE 9

| | | | MS ES+ |
|---|---|---|---|
| Interm # | Chemical Name | Structure | m/z |
| 75A | (NE,R)-N-[1-[2- Ethylsulfanyl-3- methyl-4-oxo-6- (trifluoromethyl)chromen- 8-yl]ethylidene]-2- methyl-propane-2- sulfinamide | 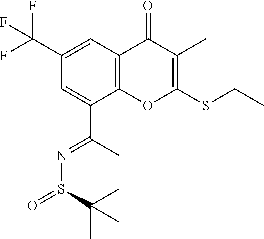 [SMILES: CCSc1oc2c(/C(C)=N/[S@](=O)C(C)(C)C)cc(C(F)(F)F)cc2c(=O)c1C] | 434 [M + H]⁺ |

### Intermediate 76A: (R)—N-[(1R)-1-(2-Ethylsulfanyl-6-methyl-4-oxo-chromen-8-yl)ethyl]-2-methyl-propane-2-sulfinamide

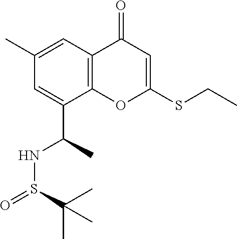 [SMILES: CCSc1cc(=O)c2cc(C)cc([C@@H](C)N[S@](=O)C(C)(C)C)c2o1]

A mixture of (NE,R)—N-[1-(2-ethylsulfanyl-6-methyl-4-oxo-chromen-8-yl)ethylidene]-2-methyl-propane-2-sulfinamide (12.0 g, 32.8 mmol) in DCM (100 mL) and MeOH (100 mL) was treated with AcOH (15.8 g, 262 mmol) and NaBH₃CN (6.19 g, 98.5 mmol) at −10° C., and stirred at 25° C. for 16 h. The mixture was quenched with NH₃·H₂O (250 mL) and extracted with DCM (3×200 mL). The combined organic extracts were washed with saturated aqueous sodium chloride (300 mL), dried over anhydrous Na₂SO₄, filtered, and concentrated to give the product as a solid (11 g, isomer ratio: 3/2, crude). MS ES+ m/z 368 [M+H]⁺.

### Intermediate 7A: (R)—N-[(1R)-1-(3,6-Dimethyl-4-oxo-2-phenyl-chromen-8-yl)ethyl]-2-methyl-propane-2-sulfinamide

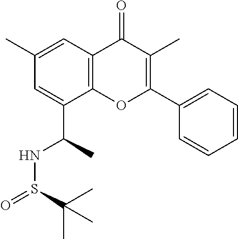 [SMILES: Cc1cc([C@@H](C)N[S@](=O)C(C)(C)C)c2oc(-c3ccccc3)c(C)c(=O)c2c1]

To a mixture of (NE,R)—N-[1-(3,6-dimethyl-4-oxo-2-phenyl-chromen-8-yl)ethylidene]-2-methyl-propane-2-sulfinamide (186 g, 470.27 mmol) and CeCl₃·7H₂O (87.61 g, 235.14 mmol, 22.35 mL) in MeOH (1600 mL) was added NaBH₄ (26.69 g, 705.41 mmol) at 15° C. The mixture was stirred at 15° C. for 1 h to give a dark suspension. The reaction was quenched with saturated aqueous NH₄Cl (1500 mL) at 15° C. Extracted with DCM (2×1500 mL), washed the combined organic phases with saturated aqueous sodium chloride (1500 mL), dried the organic phase over anhydrous Na₂SO₄, filtered, and concentrated to give the product as a yellow solid (180 g, 96%). MS ES+ m/z 398 [M+H]⁺.

### Intermediate 47C: (R)—N-[(1R)-1-Deuterio-1-(3,6-dimethyl-4-oxo-2-phenyl-chromen-8-yl)ethyl]-2-methyl-propane-2-sulfinamide

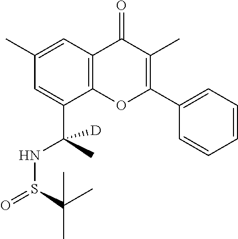 [SMILES: [2H][C@](C)(N[S@](=O)C(C)(C)C)c1cc(C)cc2c(=O)c(C)c(-c3ccccc3)oc12]

A solution of (NE,R)—N-[1-(3,6-dimethyl-4-oxo-2-phenyl-chromen-8-yl)ethylidene]-2-methyl-propane-2-sulfinamide (600 mg, 1.52 mmol) in tetradeuteromethanol (10 mL) was treated with cerium(III) chloride heptahydrate (283 mg, 0.76 mmol) and cooled to −78° C. The reaction was treated with sodium borodeuteride (127 mg, 3.03 mmol) in one portion. The reaction was allowed to stir at −78° C. After 1 h, the reaction was concentrated under reduced pressure and the residue purified by silica gel chromatography eluted with 0% to 100% EtOAc (with 25% EtOH) in heptane followed by reversed phase chromatography on C18 eluted with 0% to 100% ACN in 10 mM aqueous NH₄HCO₃ (with 5% MeOH) to give the title compound (530 mg, 88%). ES/MS m/z 399 (M+H).

### Intermediate 77A: 8-[(1R)-1-Aminoethyl]-2-ethylsulfanyl-6-methyl-chromen-4-one

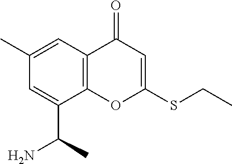 [SMILES: CCSc1cc(=O)c2cc(C)cc([C@@H](C)N)c2o1]

A mixture of (R)—N-[(1R)-1-(2-ethylsulfanyl-3,6-dimethyl-4-oxo-chromen-8-yl)ethyl]-2-methyl-propane-2-sulfinamide (6.00 g, 16.3 mmol) in EtOAc (40 mL) was treated with HCl (82 mL, 4 M in EtOAc) and stirred at 25° C. for 16 h. The mixture was concentrated, diluted with water (100 mL), and washed with EtOAc (100 mL). The pH of the aqueous phase was adjusted to 8 with NH₃·H₂O (25%) and extracted with DCM (3×100 mL). The combined organic phases were washed with saturated aqueous sodium chloride (100 mL), dried over anhydrous Na₂SO₄, filtered, and concentrated to give the product as an oil (2.4 g, crude). MS ES+ m/z 264 [M+H]⁺.

### Intermediate 48C: 8-[(1R)-1-Amino-1-deuterio-ethyl]-3,6-dimethyl-2-phenyl-chromen-4-one

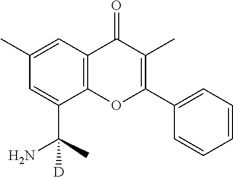 [SMILES: [2H][C@](C)(N)c1cc(C)cc2c(=O)c(C)c(-c3ccccc3)oc12]

A solution of (R)—N-[(1R)-1-deuterio-1-(3,6-dimethyl-4-oxo-2-phenyl-chromen-8-yl)ethyl]-2-methyl-propane-2-sulfinamide (530 mg, 1.33 mmol) in DCM (8 mL) was cooled to 0° C. and treated with HCl (4 N in 1,4-dioxane, 97 mg, 2.66 mmol). The reaction was allowed to stir overnight and gradually warm to rt. The reaction was diluted with DCM and washed with saturated aqueous NaHCO₃. The aqueous layer was back extracted with 25% IPA in chloroform. The organic layers were combined, dried over MgSO₄, filtered, and concentrated under reduced pressure to give the title compound which was used without purification. ES/MS m/z 295 (M+H).

### Intermediate 8A: 8-[(1R)-1-Aminoethyl]-3,6-dimethyl-2-phenyl-chromen-4-one

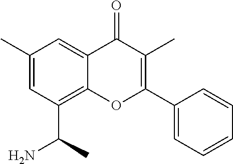 [SMILES: Cc1cc([C@@H](C)N)c2oc(-c3ccccc3)c(C)c(=O)c2c1]

A mixture of (R)—N-[(1R)-1-(3,6-dimethyl-4-oxo-2-phenyl-chromen-8-yl)ethyl]-2-methyl-propane-2-sulfinamide (180 g, 452.80 mmol) in MeOH (1500 mL) was treated with HCl/MeOH (4 M, 300 mL) and the mixture was stirred at 15° C. for 1 h to give a white suspension. Concentrated the reaction, poured the residue into water (1000 mL) and DCM (2000 mL), adjusted the pH to 12 with NH₃ in H₂O (25%), and extracted with DCM (2×1000 mL). The combined organic phases were washed with saturated aqueous sodium chloride (1000 mL), dried over anhydrous Na₂SO₄, filtered, and concentrated to give a residue. The residue was triturated with DCM (200 mL) to give the product as a white solid (122 g, 89%). MS ES+ m/z 294 [M+H]⁺.

### Intermediate 9A: (R)—N-[(1R)-1-(2-Ethylsulfanyl-3,6-dimethyl-4-oxo-chromen-8-yl)ethyl]-2-methyl-propane-2-sulfinamide

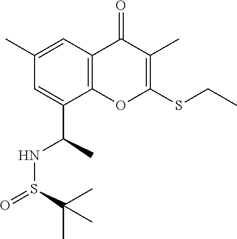 [SMILES: CCSc1oc2c([C@@H](C)N[S@](=O)C(C)(C)C)cc(C)cc2c(=O)c1C]

A mixture of 8-acetyl-2-ethylsulfanyl-3,6-dimethyl-chromen-4-one (50 g, 0.18 mol) in 500 mL of toluene was treated with (R)-2-methylpropane-2-sulfinamide (32.7 g, 0.27 mol) and titanium ethoxide (82.1 g, 0.36 mol) in one portion. The reaction was heated at 80° C. for 24 h, then cooled to rt.

The toluene solution of (NE,R)—N-[1-(2-ethylsulfanyl-3,6-dimethyl-4-oxo-chromen-8-yl)ethylidene]-2-methyl-propane-2-sulfinamide (0.18 mol) was cooled to between −10° C. and 0° C. and treated in portions with sodium borohydride (20.4 g, 0.54 mmol) keeping the internal temperature below 0° C. After stirring at −10° C. and 0° C. for 1 h, the reaction was quenched with saturated aqueous NH₄Cl. The resulting white slurry was filtered through diatomaceous earth and the solids washed with THF. The filtrate was washed with saturated aqueous sodium chloride and the organic layer concentrated onto silica gel. The material was purified by chromatography to give the product (26 g, 37% over two steps). MS ES+ m/z 382 [M+H]⁺.

The following compound in Table 10 was made in a similar way as described for (R)—N-[(1R)-1-(2-ethylsulfanyl-3,6-dimethyl-4-oxo-chromen-8-yl)ethyl]-2-methyl-propane-2-sulfinamide.

TABLE 10

| | | | MS ES+ |
|---|---|---|---|
| Interm # | Chemical Name | Structure | m/z |
| 10A | (R)-N-[(1R)-1-[2- Ethylsulfanyl-3- methyl-4-oxo-6- (trifluoromethyl)chromen- 8-yl]ethyl]-2- methyl-propane-2- sulfinamide | 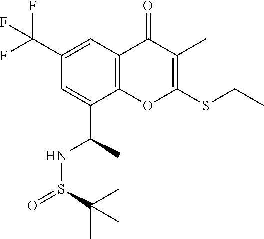 [SMILES: CCSc1oc2c([C@@H](C)N[S@](=O)C(C)(C)C)cc(C(F)(F)F)cc2c(=O)c1C] | 436 [M + H]⁺ |

### Intermediate 11A: 8-[(1R)-1-Aminoethyl]-2-ethylsulfanyl-3,6-dimethyl-chromen-4-one

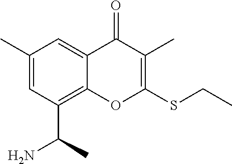 [SMILES: CCSc1oc2c([C@@H](C)N)cc(C)cc2c(=O)c1C]

A solution of (R)—N-[(1R)-1-(2-ethylsulfanyl-3,6-dimethyl-4-oxo-chromen-8-yl)ethyl]-2-methyl-propane-2-sulfinamide (20 g, 52.4 mmol) in THF (200 mL) was treated dropwise with 12 M aqueous HCl (2 eq) and stirred at rt. After 1 h, the reaction was concentrated, the residue dissolved in water, and extracted with MTBE. The pH of the aqueous phase was adjusted to between 9 and 10 with 1M aqueous NaOH and extracted with EtOAc. The organic layer was washed with saturated aqueous sodium chloride, dried over MgSO₄, filtered, and concentrated to give the product (12.6 g, 87%) as a white solid. MS ES+ m/z 278 [M+H]⁺.

The following compound in Table 11 was made in a similar way as described for 8-[(1R)-1-aminoethyl]-2-ethylsulfanyl-3,6-dimethyl-chromen-4-one.

TABLE 11

| | | | MS ES+ |
|---|---|---|---|
| Interm # | Chemical Name | Structure | m/z |
| 12A | 8-[(1R)-1- Aminoethyl]-2- ethylsulfanyl-3-methyl- 6- (trifluoromethyl)chromen- 4-one | 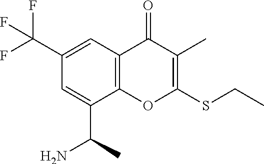 [SMILES: CCSc1oc2c([C@@H](C)N)cc(C(F)(F)F)cc2c(=O)c1C] | 332 [M + H]⁺ |

### Intermediate 13A: tert-Butyl N-[(1R)-1-(2-ethylsulfanyl-3,6-dimethyl-4-oxo-chromen-8-yl)ethyl]carbamate

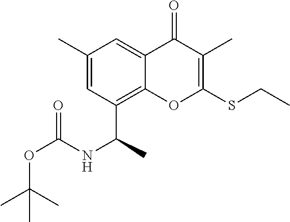 [SMILES: CCSc1oc2c([C@@H](C)NC(=O)OC(C)(C)C)cc(C)cc2c(=O)c1C]

A mixture of 8-[(1R)-1-aminoethyl]-2-ethylsulfanyl-3,6-dimethyl-chromen-4-one (2.5 g, 9.0 mmol) and diisopropylethylamine (2.0 g, 16 mmol) in DCM (40 mL) was treated with di-tert-butyl dicarbonate (3.0 g, 14 mmol) and stirred overnight at rt. Concentrated the reaction and purified the residue by silica gel chromatography eluted with 0% to 100% EtOAc in heptane to give the product (2.5 g, 73%). MS ES+ m/z 378 [M+H]⁺.

The following compounds in Table 12 were made in a similar way as described for tert-butyl N-[(1R)-1-(2-ethylsulfanyl-3,6-dimethyl-4-oxo-chromen-8-yl)ethyl]carbamate. Various methods were used to purify the compounds, which would be apparent to one skilled in the art.

TABLE 12

| | | | MS ES+ |
|---|---|---|---|
| Interm # | Chemical Name | Structure | m/z |
| 78A | tert-Butyl N-[(1R)-1- [2-ethylsulfanyl-3- methyl-4-oxo-6- (trifluoromethyl)chromen- 8- yl]ethyl]carbamate | 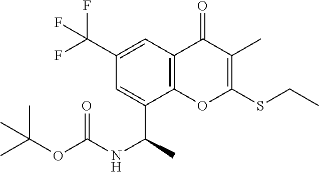 [SMILES: CCSc1oc2c([C@@H](C)NC(=O)OC(C)(C)C)cc(C(F)(F)F)cc2c(=O)c1C] | 432 [M + H]⁺ |
| 79A | tert-Butyl N-[(1R)-1- (2-ethylsulfanyl-6- methyl-4-oxo- chromen-8- yl)ethyl]carbamate | 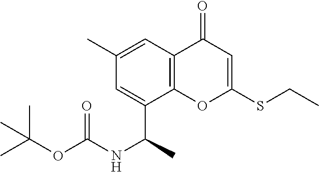 [SMILES: CCSc1cc(=O)c2cc(C)cc([C@@H](C)NC(=O)OC(C)(C)C)c2o1] | 364 [M + H]⁺ |

### Intermediate 49C: tert-Butyl N-[(1R)-1-(2-ethylsulfanyl-3-iodo-6-methyl-4-oxo-chromen-8-yl)ethyl]carbamate

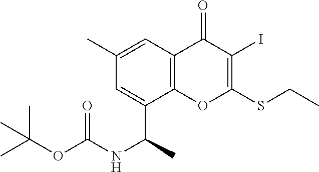 [SMILES: CCSc1oc2c([C@@H](C)NC(=O)OC(C)(C)C)cc(C)cc2c(=O)c1I]

tert-Butyl N-[(1R)-1-(2-ethylsulfanyl-6-methyl-4-oxo-chromen-8-yl)ethyl]carbamate (5.00 g, 13.8 mmol) was dissolved in dry THF (27.5 mL) and cooled to 0° C. When cool, the reaction was treated dropwise with 2,2,6,6-tetramethylpiperidinylzinc chloride lithium chloride complex (41.3 mL, 41.3 mmol, 1M in THF) over 15 min. The reaction was stirred at 0° C. for 1 h. A solution of iodine (6.98 g, 27.5 mmol) in THF (20 mL) was added dropwise and the reaction stirred an additional 30 min. The reaction was concentrated under reduced pressure and the residue partitioned between water and DCM. The layers were separated and the aqueous layer re-extracted with DCM (2×). The organic layers were combined, washed with saturated aqueous Na₂S₂O₃, dried over Na₂SO₄, filtered, and concentrated under reduced pressure. The residue was purified by silica gel chromatography eluted with 0% to 100% EtOAc in heptane to give the title compound (8.33 g, 124%) as a brown foam. ES/MS m/z 490 (M+H).

### Intermediate 50C: tert-Butyl N-[(1R)-1-(2-ethylsulfanyl-6-methyl-3-oxazol-4-yl-4-oxo-chromen-8-yl)ethyl]carbamate

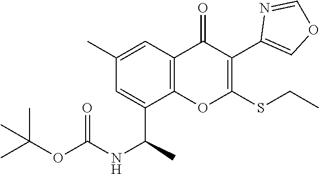 [SMILES: CCSc1oc2c([C@@H](C)NC(=O)OC(C)(C)C)cc(C)cc2c(=O)c1-c1cocn1]

Transferred tert-butyl N-[(1R)-1-(2-ethylsulfanyl-3-iodo-6-methyl-4-oxo-chromen-8-yl)ethyl]carbamate (1.0 g, 2.04 mmol) and oxazol-4-ylboronic acid (0.46 g, 4.09 mmol) to a tube. Added 1,1′-bis(di-tert-butylphosphino)ferrocene palladium dichloride (0.33 g, 0.51 mmol) and cesium carbonate (2.0 g, 6.13 mmol) suspended in 9:1 1,4-dioxane/water (4.1 mL). The reaction was sparged with argon gas for 5 min and then stirred at 65° C. for 2 h. After cooling to rt, the reaction was filtered through diatomaceous earth and the solids washed with DCM. The filtrate was concentrated under reduced pressure and the residue purified by silica gel chromatography eluted with 0% to 40% EtOAc in heptane to give the title compound (0.29 g, 33%). ES/MS m/z 431 (M+H).

### Intermediate 80A: 8-[(1R)-1-Aminoethyl]-3,6-dimethyl-2-(2-methylindazol-5-yl)chromen-4-one

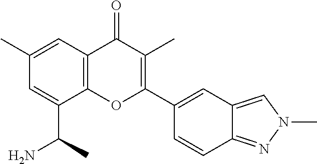

Charged a 20 mL vial with 8-[(1R)-1-aminoethyl]-2-ethylsulfanyl-3,6-dimethyl-chromen-4-one (0.30 g, 1.08 mmol), (2-methylindazol-5-yl)boronic acid (0.57 g, 3.24 mmol), tetrakis(triphenylphosphine)palladium(0) (0.13 g, 0.11 mmol), copper(I) thiophene-2-carboxylate (0.31 g, 1.62 mmol), and EtOH (15 mL). Degassed the reaction with argon, capped the vial, and stirred at 55° C. for 16 h. The reaction mixture was loaded onto celite and purified by reverse phase C18 flash chromatography eluted with 1000 to 100% ACN in 10 mM aqueous NH₄HCO₃ with 500 MeOH to give the title compound (0.23 g, 600%). MS ES+ m/z 348 [M+H]⁺.

The following compounds in Table 13 were made in a similar way as described for 8-[(1R)-1-aminoethyl]-3,6-dimethyl-2-(2-methylindazol-5-yl)chromen-4-one. Various methods were used to purify the compounds, which would be apparent to one skilled in the art.

TABLE 13

| | | | MS ES+ |
|---|---|---|---|
| Interm # | Chemical Name | Structure | m/z |
| 81A | 8-[(1R)-1-Aminoethyl]-2- (3,4-difluorophenyl)-3,6- dimethyl-chromen-4-one | 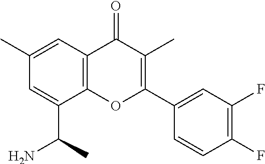 [SMILES: Cc1cc([C@@H](C)N)c2oc(-c3ccc(F)c(F)c3)c(C)c(=O)c2c1] | 330 [M + H]⁺ |
| 82A | 8-[(1R)-1-Aminoethyl]-2- (5-fluoro-3-pyridyl)-3,6- dimethyl-chromen-4-one | 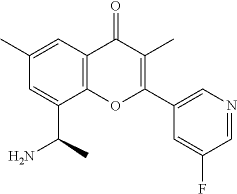 [SMILES: Cc1cc([C@@H](C)N)c2oc(-c3cncc(F)c3)c(C)c(=O)c2c1] | 313 [M + H]⁺ |
| 83A | 8-[(1R)-1-Aminoethyl]-2- (3-fluorophenyl)-3,6- dimethyl-chromen-4-one | 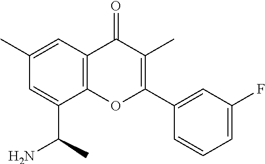 [SMILES: Cc1cc([C@@H](C)N)c2oc(-c3cccc(F)c3)c(C)c(=O)c2c1] | 312 [M + H]⁺ |
| 84A | tert-Butyl N-[(1R)-1-[2-(2- fluorophenyl)-3,6-dimethyl- 4-oxo-chromen-8- yl]ethyl]carbamate | 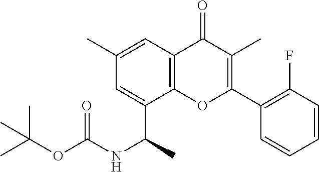 [SMILES: Cc1cc([C@@H](C)NC(=O)OC(C)(C)C)c2oc(-c3ccccc3F)c(C)c(=O)c2c1] | 412 [M + H]⁺ |
| 85A | 8-[(1R)-1-Aminoethyl]-3,6- dimethyl-2-(3- pyridyl)chromen-4-one | 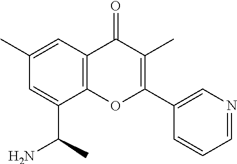 [SMILES: Cc1cc([C@@H](C)N)c2oc(-c3cccnc3)c(C)c(=O)c2c1] | 295 [M + H]⁺ |
| 86A | 8-[(1R)-1-Aminoethyl]-2- (2-fluorophenyl)-3,6- dimethyl-chromen-4-one | 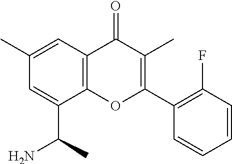 [SMILES: Cc1cc([C@@H](C)N)c2oc(-c3ccccc3F)c(C)c(=O)c2c1] | 312 [M + H]⁺ |
| 87A | 8-[(1R)-1-Aminoethyl]-3,6- dimethyl-2-(1- methylpyrazol-4- yl)chromen-4-one | 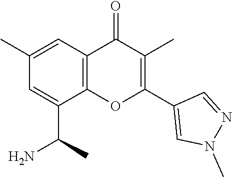 [SMILES: Cc1cc([C@@H](C)N)c2oc(-c3cnn(C)c3)c(C)c(=O)c2c1] | 298 [M + H]⁺ |
| 88A | 8-[(1R)-1-Aminoethyl]-2- [1-(difluoromethyl)pyrazol- 4-yl]-3,6-dimethyl- chromen-4-one | 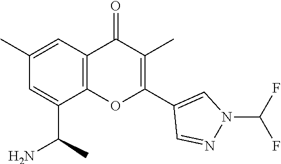 [SMILES: Cc1cc([C@@H](C)N)c2oc(-c3cnn(C(F)F)c3)c(C)c(=O)c2c1] | 334 [M + H]⁺ |
| 89A | tert-Butyl N-[(1R)-1-[2-(2- fluoro-3-pyridyl)-3,6- dimethyl-4-oxo-chromen-8- yl]ethyl]carbamate | 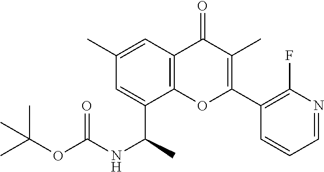 [SMILES: Cc1cc([C@@H](C)NC(=O)OC(C)(C)C)c2oc(-c3cccnc3F)c(C)c(=O)c2c1] | 413 [M + H]⁺ |
| 90A | tert-Butyl N-[(1R)-1-[2- (2,3-difluorophenyl)-3,6- dimethyl-4-oxo-chromen-8- yl]ethyl]carbamate | 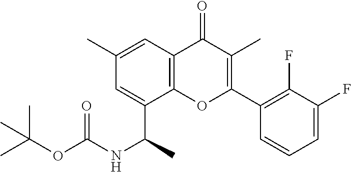 [SMILES: Cc1cc([C@@H](C)NC(=O)OC(C)(C)C)c2oc(-c3cccc(F)c3F)c(C)c(=O)c2c1] | 430 [M + H]⁺ |
| 91A | 8-[(1R)-1-Aminoethyl]-2- [6-(difluoromethyl)-2- pyridyl]-3,6-dimethyl- chromen-4-one | 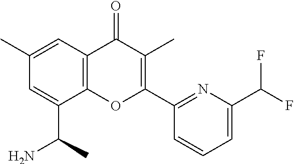 [SMILES: Cc1cc([C@@H](C)N)c2oc(-c3cccc(C(F)F)n3)c(C)c(=O)c2c1] | 345 [M + H]⁺ |
| 92A | tert-Butyl N-[(1R)-1-[2- (2,4-difluorophenyl)-3,6- dimethyl-4-oxo-chromen-8- yl]ethyl]carbamate | 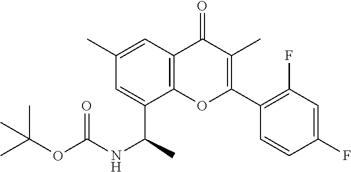 [SMILES: Cc1cc([C@@H](C)NC(=O)OC(C)(C)C)c2oc(-c3ccc(F)cc3F)c(C)c(=O)c2c1] | 430 [M + H]⁺ |
| 93A | tert-Butyl N-[(1R)-1-[2-(6- fluoro-2-pyridyl)-3,6- dimethyl-4-oxo-chromen-8- yl]ethyl]carbamate | 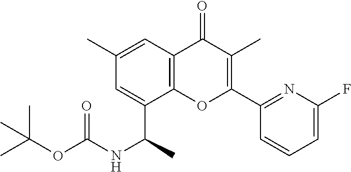 [SMILES: Cc1cc([C@@H](C)NC(=O)OC(C)(C)C)c2oc(-c3cccc(F)n3)c(C)c(=O)c2c1] | 413 [M + H]⁺ |
| 94A | tert-Butyl N-[(1R)-1-[2-(3- fluoro-2-pyridyl)-3,6- dimethyl-4-oxo-chromen-8- yl]ethyl]carbamate | 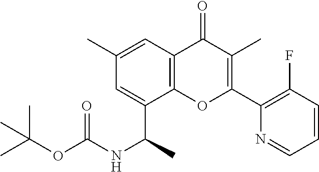 [SMILES: Cc1cc([C@@H](C)NC(=O)OC(C)(C)C)c2oc(-c3ncccc3F)c(C)c(=O)c2c1] | 413 [M + H]⁺ |
| 95A | tert-Butyl N-[(1R)-1-[2- (2,5-difluoro-3-pyridyl)-3,6- dimethyl-4-oxo-chromen-8- yl]ethyl]carbamate | 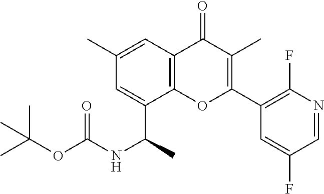 [SMILES: Cc1cc([C@@H](C)NC(=O)OC(C)(C)C)c2oc(-c3cc(F)cnc3F)c(C)c(=O)c2c1] | 431 [M + H]⁺ |
| 96A | tert-Butyl N-[(1R)-1-[2-(6- methoxy-2-pyridyl)-3,6- dimethyl-4-oxo-chromen-8- yl]ethyl]carbamate | 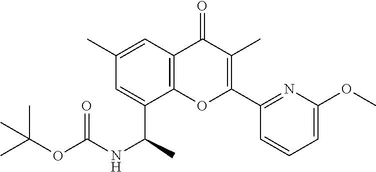 [SMILES: COc1cccc(-c2oc3c([C@@H](C)NC(=O)OC(C)(C)C)cc(C)cc3c(=O)c2C)n1] | 425 [M + H]⁺ |
| 97A | 8-[(1R)-1-Aminoethyl]-3- methyl-2-(1-methylpyrazol- 4-yl)-6- (trifluoromethyl)chromen-4- one | 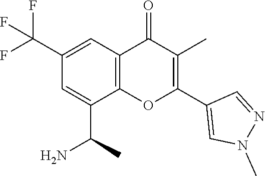 [SMILES: Cc1c(-c2cnn(C)c2)oc2c([C@@H](C)N)cc(C(F)(F)F)cc2c1=O] | 352 [M + H]⁺ |
| 98A | tert-Butyl N-[(1R)-1-[3- methyl-4-oxo-2-(2-pyridyl)- 6-(trifluoromethyl)chromen- 8-yl]ethyl]carbamate | 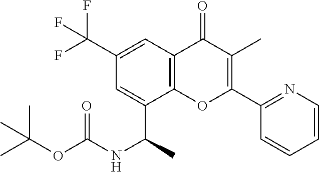 [SMILES: Cc1c(-c2ccccn2)oc2c([C@@H](C)NC(=O)OC(C)(C)C)cc(C(F)(F)F)cc2c1=O] | 449 [M + H]⁺ |
| 99A | tert-Butyl N-[(1R)-1-[6- methyl-2-(1-methylpyrazol- 4-yl)-4-oxo-chromen-8- yl]ethyl]carbamate | 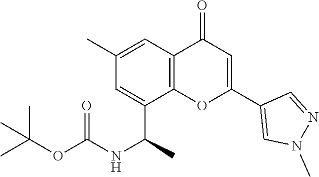 [SMILES: Cc1cc([C@@H](C)NC(=O)OC(C)(C)C)c2oc(-c3cnn(C)c3)cc(=O)c2c1] | 384 [M + H]⁺ |
| 100A | 8-[(1R)-1-Aminoethyl]-2- (5-fluoro-3-pyridyl)-3- methyl-6- (trifluoromethyl)chromen-4- one | 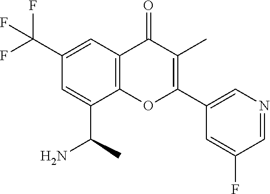 [SMILES: Cc1c(-c2cncc(F)c2)oc2c([C@@H](C)N)cc(C(F)(F)F)cc2c1=O] | 367 [M + H]⁺ |
| 101A | 8-[(1R)-1-Aminoethyl]-3,6- dimethyl-2-pyrimidin-5-yl- chromen-4-one | 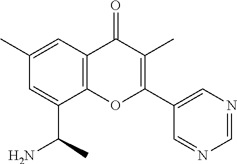 [SMILES: Cc1cc([C@@H](C)N)c2oc(-c3cncnc3)c(C)c(=O)c2c1] | 279 [MH − NH₃]⁺ |
| 51C | 8-[(1R)-1-Aminoethyl]-2- (2,5-dimethylpyrazol-3-yl)- 3,6-dimethyl-chromen-4- one | 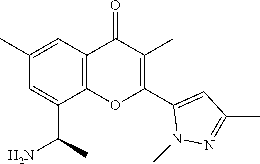 | 312 [M + H]⁺ |
| 52C | 8-[(1R)-1-Aminoethyl]-2- [1-[2-[tert- butyl(dimethyl)silyl]oxyethyl] pyrazol-4-yl]-3,6- dimethyl-chromen-4-one | 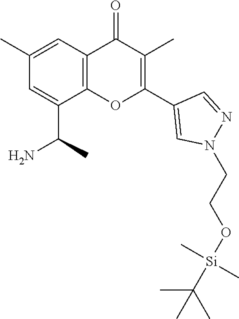 | 442 [M + H]⁺ |
| 53C | 8-[(1R)-1-Aminoethyl]-3,6- dimethyl-2-(2- methyltriazol-4-yl)chromen- 4-one | 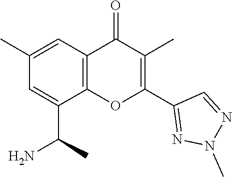 [SMILES: Cc1cc([C@@H](C)N)c2oc(-c3cnn(C)n3)c(C)c(=O)c2c1] | 299 [M + H]⁺ |
| 54C | tert-Butyl N-[(1R)-1-[2- (1,5-dimethyl-6-oxo-3- pyridyl)-6-methyl-4-oxo- chromen-8- yl]ethyl]carbamate | 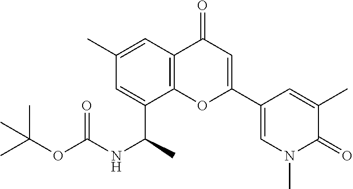 [SMILES: Cc1cc([C@@H](C)NC(=O)OC(C)(C)C)c2oc(-c3cc(C)c(=O)n(C)c3)cc(=O)c2c1] | 425 [M + H]⁺ |
| 55C | 8-[(1R)-1-Aminoethyl]-2- (3-fluoro-1-methyl-pyrazol- 4-yl)-3,6-dimethyl- chromen-4-one | 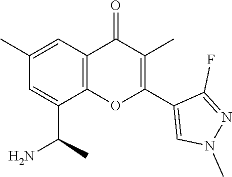 [SMILES: Cc1cc([C@@H](C)N)c2oc(-c3cn(C)nc3F)c(C)c(=O)c2c1] | 316 [M + H]⁺ |
| 56C | tert-Butyl N-[(1R)-1-(6- methyl-4-oxo-2-phenyl- chromen-8- yl)ethyl]carbamate | 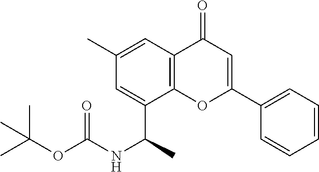 [SMILES: Cc1cc([C@@H](C)NC(=O)OC(C)(C)C)c2oc(-c3ccccc3)cc(=O)c2c1] | 380 [M +H]⁺ |
| 57C | tert-Butyl N-[(1R)-1-[2-(1- isopropyl-6-oxo-3-pyridyl)- 3,6-dimethyl-4-oxo- chromen-8- yl]ethyl]carbamate | 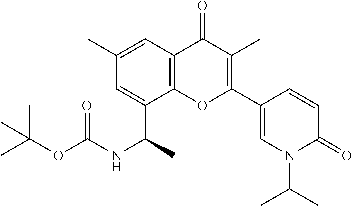 [SMILES: Cc1cc([C@@H](C)NC(=O)OC(C)(C)C)c2oc(-c3ccc(=O)n(C(C)C)c3)c(C)c(=O)c2c1] | 453 [M + H]⁺ |
| 58C | tert-Butyl N-[(1R)-1-[6- methyl-2-(1-methyl-6-oxo- 3-pyridyl)-4-oxo-chromen- 8-yl]ethyl]carbamate | 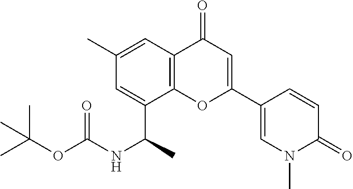 [SMILES: Cc1cc([C@@H](C)NC(=O)OC(C)(C)C)c2oc(-c3ccc(=O)n(C)c3)cc(=O)c2c1] | 411 [M + H]⁺ |
| 59C | tert-Butyl N-[(1R)-1-(6- methyl-3-oxazol-4-yl-4- oxo-2-phenyl-chromen-8- yl)ethyl]carbamate | 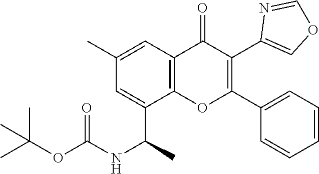 [SMILES: Cc1cc([C@@H](C)NC(=O)OC(C)(C)C)c2oc(-c3ccccc3)c(-c3cocn3)c(=O)c2c1] | 447 [M + H]⁺ |
| 60C | 8-[(1R)-1-Aminoethyl]-2- [1-(2- methoxyethyl)pyrazol-4-yl]- 3,6-dimethyl-chromen-4- one | 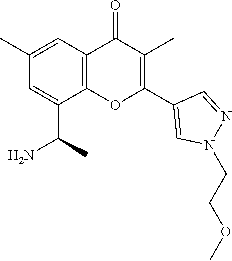 [SMILES: COCCn1cc(-c2oc3c([C@@H](C)N)cc(C)cc3c(=O)c2C)cn1] | 342 [M + H]⁺ |
| 61C | 8-[(1R)-1-Aminoethyl]-2- (1,5-dimethylpyrazol-4-yl)- 3,6-dimethyl-chromen-4- one | 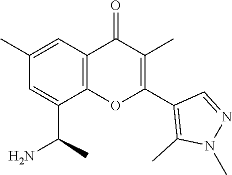 | 312 [M + H]⁺ |
| 62C | 2-[4-[8-[(1R)-1- Aminoethyl]-3,6-dimethyl- 4-oxo-chromen-2- yl]pyrazol-1-yl]-2-methyl- propanenitrile | 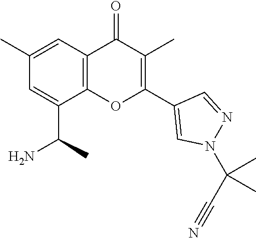 [SMILES: Cc1cc([C@@H](C)N)c2oc(-c3cnn(C(C)(C)C#N)c3)c(C)c(=O)c2c1] | 351 [M + H]⁺ |
| 63C | tert-Butyl N-[(1R)-1-[6- methyl-2-(1-methylpyrazol- 4-yl)-3-oxazol-4-yl-4-oxo- chromen-8- yl]ethyl]carbamate | 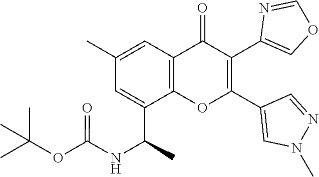 [SMILES: Cc1cc([C@@H](C)NC(=O)OC(C)(C)C)c2oc(-c3cnn(C)c3)c(-c3cocn3)c(=O)c2c1] | 451 [M + H]⁺ |
| 64C | tert-Butyl N-[(1R)-1-[3,6- dimethyl-2-(1-methyl-2- oxo-4-pyridyl)-4-oxo- chromen-8- yl]ethyl]carbamate | 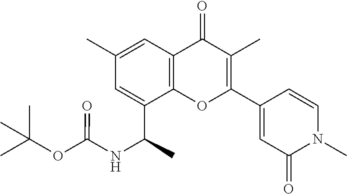 [SMILES: Cc1cc([C@@H](C)NC(=O)OC(C)(C)C)c2oc(-c3ccn(C)c(=O)c3)c(C)c(=O)c2c1] | 425 [M + H]⁺ |
| 65C | tert-Butyl N-[(1R)-1-[3,6- dimethyl-2-(1-methyl-6- oxo-3-pyridyl)-4-oxo- chromen-8- yl]ethyl]carbamate | 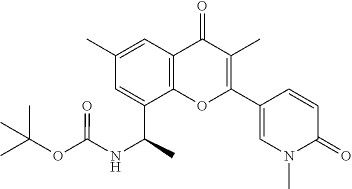 [SMILES: Cc1cc([C@@H](C)NC(=O)OC(C)(C)C)c2oc(-c3ccc(=O)n(C)c3)c(C)c(=O)c2c1] | 425 [M + H]⁺ |
| 66C | tert-Butyl N-[(1R)-1-[3,6- dimethyl-2-(1- methylpyrazolo[4,3- b]pyridin-5-yl)-4-oxo- chromen-8- yl]ethyl]carbamate | 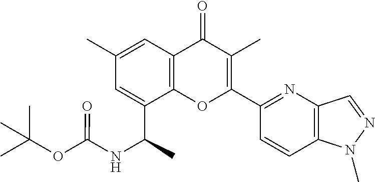 [SMILES: Cc1cc([C@@H](C)NC(=O)OC(C)(C)C)c2oc(-c3ccc4c(cnn4C)n3)c(C)c(=O)c2c1] | 449 [M + H]⁺ |
| 67C | 8-[(1R)-1-Aminoethyl]-3,6- dimethyl-2-(2- methylpyrazol-3- yl)chromen-4-one | 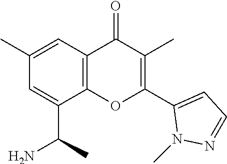 [SMILES: Cc1cc([C@@H](C)N)c2oc(-c3ccnn3C)c(C)c(=O)c2c1] | 298 [M + H]⁺ |
| 68C | 8-[(1R)-1-Aminoethyl]-2- (1-isopropylpyrazol-4-yl)- 3,6-dimethyl-chromen-4- one | 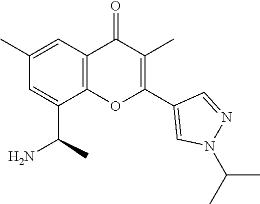 [SMILES: Cc1cc([C@@H](C)N)c2oc(-c3cnn(C(C)C)c3)c(C)c(=O)c2c1] | 326 [M + H]⁺ |
| 69C | 8-[(1R)-1-Aminoethyl]-2- (1-cyclopropylpyrazol-4- yl)-3,6-dimethyl-chromen- 4-one | 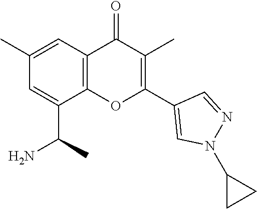 [SMILES: Cc1cc([C@@H](C)N)c2oc(-c3cnn(C4CC4)c3)c(C)c(=O)c2c1] | 324 [M + H]⁺ |
| 70C | tert-Butyl N-[(1R)-1-[2-(5- fluoro-2-pyridyl)-3,6- dimethyl-4-oxo-chromen-8- yl]ethyl]carbamate | 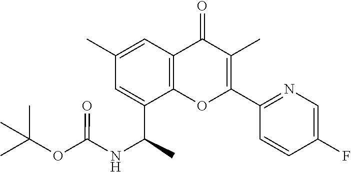 [SMILES: Cc1cc([C@@H](C)NC(=O)OC(C)(C)C)c2oc(-c3ccc(F)cn3)c(C)c(=O)c2c1] | 413 [M + H]⁺ |
| 71C | tert-Butyl N-[(1R)-1-[6- methyl-2-(2-methylindazol- 5-yl)-4-oxo-chromen-8- yl]ethyl]carbamate | 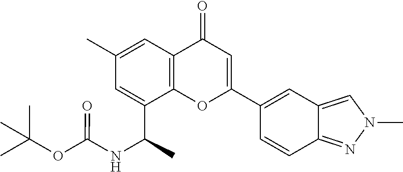 [SMILES: Cc1cc([C@@H](C)NC(=O)OC(C)(C)C)c2oc(-c3ccc4nn(C)cc4c3)cc(=O)c2c1] | 434 [M + H]⁺ |
| 72C | tert-Butyl N-[(1R)-1-[3,6- dimethyl-2-(1- methylpyrazol-4-yl)-4-oxo- chromen-8- yl]ethyl]carbamate | 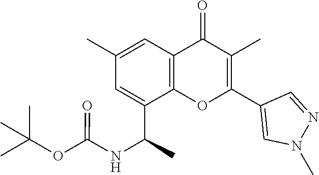 [SMILES: Cc1cc([C@@H](C)NC(=O)OC(C)(C)C)c2oc(-c3cnn(C)c3)c(C)c(=O)c2c1] | 398 [M + H]⁺ |
| 73C | 8-[(1R)-1-Aminoethyl]-3,6- dimethyl-2-[1- (trideuteriomethyl)pyrazol- 4-yl]chromen-4-one | 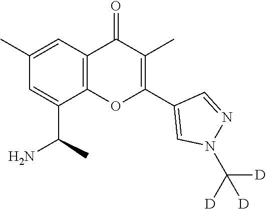 [SMILES: [2H]C([2H])([2H])n1cc(-c2oc3c([C@@H](C)N)cc(C)cc3c(=O)c2C)cn1] | 301 [M + H]⁺ |
| 74C | tert-Butyl N-[(1R)-1-[3,6- dimethyl-2-(2- methylindazol-5-yl)-4-oxo- chromen-8- yl]ethyl]carbamate | 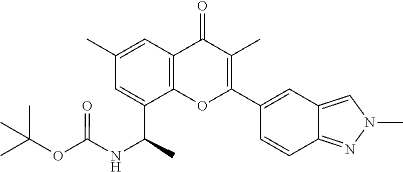 [SMILES: Cc1cc([C@@H](C)NC(=O)OC(C)(C)C)c2oc(-c3ccc4nn(C)cc4c3)c(C)c(=O)c2c1] | 448 [M + H]⁺ |
| 75C | tert-Butyl N-[(1R)-1-[2-(6- methoxy-2-pyridyl)-3,6- dimethyl-4-oxo-chromen-8- yl]ethyl]carbamate | 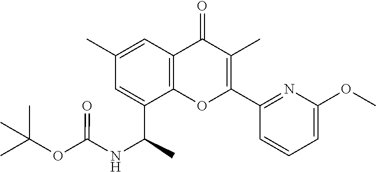 [SMILES: COc1cccc(-c2oc3c([C@@H](C)NC(=O)OC(C)(C)C)cc(C)cc3c(=O)c2C)n1] | 425 [M + H]⁺ |
| 76C¹ | tert-Butyl N-[(1R)-1-[3,6- dimethyl-2-(2- methylpyrazolo[4,3- b]pyridin-5-yl)-4-oxo- chromen-8- yl]ethyl]carbamate | 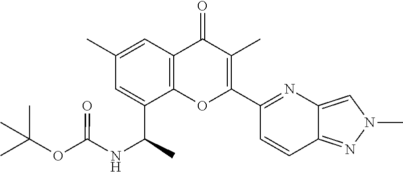 [SMILES: Cc1cc([C@@H](C)NC(=O)OC(C)(C)C)c2oc(-c3ccc4nn(C)cc4n3)c(C)c(=O)c2c1] | 449 [M + H]⁺ |
| 77C | tert-Butyl N-[(1R)-1-[6- methyl-4-oxo-2-(2- pyridyl)chromen-8- yl]ethyl]carbamate | 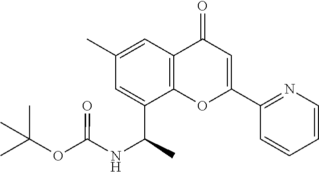 [SMILES: Cc1cc([C@@H](C)NC(=O)OC(C)(C)C)c2oc(-c3ccccn3)cc(=O)c2c1] | 381 [M + H]⁺ |
| 78C | 8-[(1R)-1-Aminoethyl]-3,6- dimethyl-2-(1- methylindazol-5- yl)chromen-4-one | 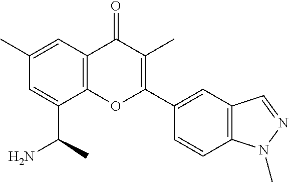 [SMILES: Cc1cc([C@@H](C)N)c2oc(-c3ccc4c(cnn4C)c3)c(C)c(=O)c2c1] | 348 [M + H]⁺ |
| 79C | tert-Butyl N-[(1R)-1-[3,6- dimethyl-2-(2- methylpyrazolo[3,4- b]pyridin-5-yl)-4-oxo- chromen-8- yl]ethyl]carbamate | 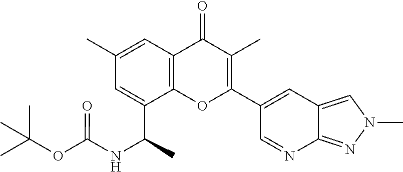 [SMILES: Cc1cc([C@@H](C)NC(=O)OC(C)(C)C)c2oc(-c3cnc4nn(C)cc4c3)c(C)c(=O)c2c1] | 449 [M + H]⁺ |
| 80C | tert-Butyl N-[(1R)-1-[6- methyl-4-oxo-2-(3- pyridyl)chromen-8- yl]ethyl]carbamate | 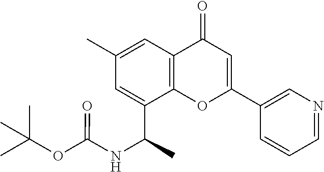 [SMILES: Cc1cc([C@@H](C)NC(=O)OC(C)(C)C)c2oc(-c3cccnc3)cc(=O)c2c1] | 381 [M + H]⁺ |
| 81C | 8-[(1R)-1-Aminoethyl]-2- (6,7-dihydro-4H- pyrazolo[5,1-c][1,4]oxazin- 3-yl)-3,6-dimethyl- chromen-4-one | 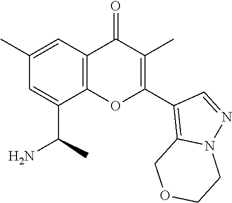 [SMILES: Cc1cc([C@@H](C)N)c2oc(-c3cnn4c3COCC4)c(C)c(=O)c2c1] | 340 [M + H]⁺ |
| 82C | 8-[(1R)-1-Aminoethyl]-3,6- dimethyl-2-[1-[(3- methyloxetan-3- yl)methyl]pyrazol-4- yl]chromen-4-one | 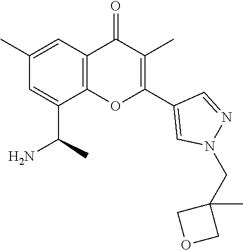 | 368 [M + H]⁺ |
| 83C | tert-Butyl 3-[4-[8-[(1R)-1- aminoethyl]-3,6-dimethyl-4- oxo-chromen-2-yl]pyrazol- 1-yl]azetidine-1-carboxylate | 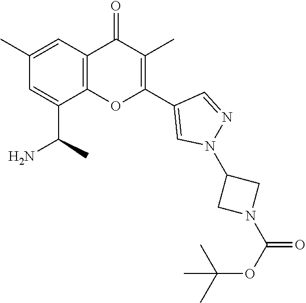 [SMILES: Cc1cc([C@@H](C)N)c2oc(-c3cnn(C4CN(C(=O)OC(C)(C)C)C4)c3)c(C)c(=O)c2c1] | 439 [M + H]⁺ |
| 84C | tert-Butyl 3-[8-[(1R)-1- aminoethyl]-3,6-dimethyl-4- oxo-chromen-2-yl]-6,7- dihydro-4H-pyrazolo[1,5- a]pyrazine-5-carboxylate | 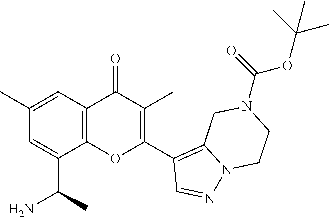 [SMILES: Cc1cc([C@@H](C)N)c2oc(-c3cnn4c3CN(C(=O)OC(C)(C)C)CC4)c(C)c(=O)c2c1] | 439 [M + H]⁺ |
| 85C | 8-[(1R)-1-Aminoethyl]-3,6- dimethyl-2-pyrazolo[1,5- a]pyrimidin-3-yl-chromen- 4-one | 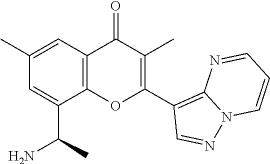 [SMILES: Cc1cc([C@@H](C)N)c2oc(-c3cnn4cccnc34)c(C)c(=O)c2c1] | 335 [M + H]⁺ |

¹Stananne used as coupling partner.

### Intermediate 86C: 8-[(1R)-1-Aminoethyl]-3,6-dimethyl-2-[1-(oxetan-3-yl)pyrazol-4-yl]chromen-4-one

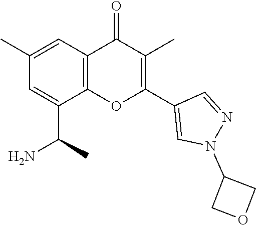 [SMILES: Cc1cc([C@@H](C)N)c2oc(-c3cnn(C4COC4)c3)c(C)c(=O)c2c1]

A vial was charged with 8-[(1R)-1-aminoethyl]-2-ethylsulfanyl-3,6-dimethyl-chromen-4-one (0.50 g, 1.80 mmol), 1-(oxetan-3-yl)-4-(4,4,5,5-tetramethyl-1,3,2-dioxaborolan-2-yl)pyrazole (1.35 g, 5.41 mmol), tetrakis(triphenylphosphine)palladium(0) (0.52 g, 0.45 mmol), copper(I) 3-methylsalicylate (0.58 g, 2.70 mmol), and EtOH (8 mL). Nitrogen was bubbled through the reaction for 2 min, the vial was sealed, and the reaction stirred at 60° C. for 16 h. The reaction was retreated with the same amounts of 1-(oxetan-3-yl)-4-(4,4,5,5-tetramethyl-1,3,2-dioxaborolan-2-yl)pyrazole, tetrakis(triphenylphosphine)palladium(0), and copper(I) 3-methylsalicylate, and nitrogen was bubbled through the reaction for 5 min. The reaction was heated at 60° C. for 16 h. The reaction was cooled, filtered through diatomaceous earth, solids washed with DCM/MeOH, and the filtrate concentrated under reduced pressure. The residue was purified by reversed phase chromatography on C18 eluted with 10% to 65% ACN in 10 mM aqueous NH₄HCO₃ to give the title compound (0.31 g, 51%). ES/MS m/z 340 (M+H).

The following compounds in Table 14 were made in a similar way as described for 8-[(1R)-1-aminoethyl]-3,6-dimethyl-2-[1-(oxetan-3-yl)pyrazol-4-yl]chromen-4-one. Various methods were used to purify the compounds, which would be apparent to one skilled in the art.

TABLE 14

| | | | MS ES+ |
|---|---|---|---|
| Interm # | Chemical Name | Structure | m/z |
| 87C | 8-[(1R)-1-Aminoethyl]- 3,6-dimethyl-2-(1- methylpyrazol-4- yl)chromen-4-one | 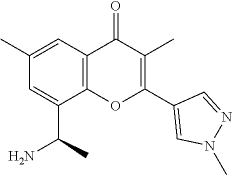 | 298 [M + H]⁺ |
| 88C | tert-Butyl N-[(1R)-1-(3,6- dimethyl-4-oxo-2-pyrazin- 2-yl-chromen-8- yl)ethyl]carbamate | 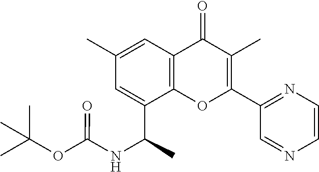 [SMILES: Cc1cc([C@@H](C)NC(=O)OC(C)(C)C)c2oc(-c3cnccn3)c(C)c(=O)c2c1] | 396 [M + H]⁺ |
| 89C | tert-Butyl 6-chloro-3- [[(1R)-1-[2-[6-(1- cyanocyclopropyl)-3- pyridyl]-3,6-dimethyl-4- oxo-chromen-8- yl]ethyl]amino]pyridine- 2-carboxylate | 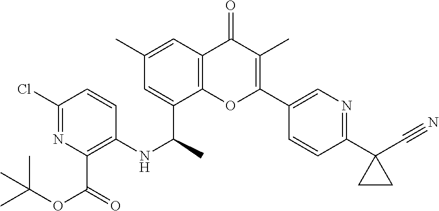 | 189 [M + H]⁺ |
| 90C | tert-Butyl N-[(1R)-1-[2- (3,6-difluoro-2-pyridyl)- 3,6-dimethyl-4-oxo- chromen-8- yl]ethyl]carbamate | 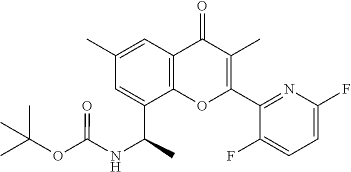 [SMILES: Cc1cc([C@@H](C)NC(=O)OC(C)(C)C)c2oc(-c3nc(F)ccc3F)c(C)c(=O)c2c1] | 431 [M + H]⁺ |
| 91C¹ | tert-Butyl N-[(1R)-1-[2- (4-fluoro-3-pyridyl)-3,6- dimethyl-4-oxo-chromen- 8-yl]ethyl]carbamate | 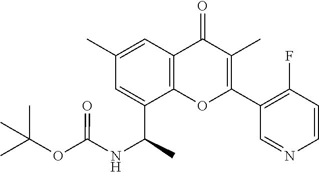 [SMILES: Cc1cc([C@@H](C)NC(=O)OC(C)(C)C)c2oc(-c3cnccc3F)c(C)c(=O)c2c1] | 413 [M + H]⁺ |
| 92C¹ | tert-Butyl N-[(1R)-1-(3,6- dimethyl-4-oxo-2- pyrimidin-2-yl-chromen- 8-yl)ethyl]carbamate | 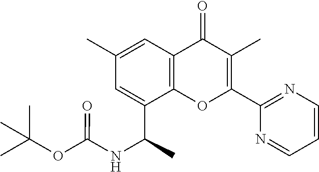 [SMILES: Cc1cc([C@@H](C)NC(=O)OC(C)(C)C)c2oc(-c3ncccn3)c(C)c(=O)c2c1] | 396 [M + H]⁺ |
| 93C¹ | tert-Butyl N-[(1R)-1-[2- (4-fluoro-2-pyridyl)-3,6- dimethyl-4-oxo-chromen- 8-yl]ethyl]carbamate | 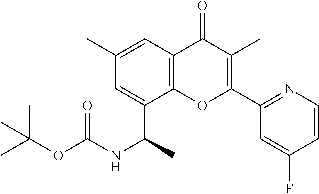 [SMILES: Cc1cc([C@@H](C)NC(=O)OC(C)(C)C)c2oc(-c3cc(F)ccn3)c(C)c(=O)c2c1] | 413 [M + H]⁺ |

¹Stannane used as coupling partner.

### Intermediate 102A: tert-Butyl N-[(1R)-1-[3,6-dimethyl-4-oxo-2-(2-pyridyl)chromen-8-yl]ethyl]carbamate

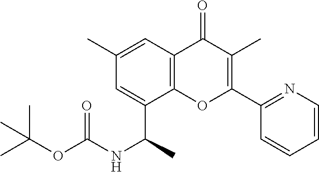 [SMILES: Cc1cc([C@@H](C)NC(=O)OC(C)(C)C)c2oc(-c3ccccn3)c(C)c(=O)c2c1]

Combined tert-butyl N-[(1R)-1-(2-ethylsulfanyl-3,6-dimethyl-4-oxo-chromen-8-yl)ethyl]carbamate (0.50 g, 1.32 mmol), copper(I) thiophene-2-carboxylate (0.63 g, 3.31 mmol), triphenylphosphine (0.28 g, 1.06 mmol), tris(dibenzylideneacetone)dipalladium(0) (0.12 g, 0.13 mmol), and pyridine-2-ylboronic acid (0.37 g, 3.05 mmol) in 8 mL of EtOH and degassed with argon for 5 min. The reaction was allowed to stir at 35° C. for 72 h. The reaction was recharged with 300 mg of all reagents, degassed, and allowed to stir at 35° C. overnight. The reaction was filtered and purified by silica gel chromatography eluted with 0% to 100% EtOAc in heptane to give the title compound (0.50 g, 96%). MS ES− m/z 395 [M−H]⁻.

### Intermediate 103A: 8-[(1R)-1-Aminoethyl]-2-(2,6-difluorophenyl)-3,6-dimethyl-chromen-4-one

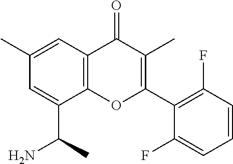 [SMILES: Cc1cc([C@@H](C)N)c2oc(-c3c(F)cccc3F)c(C)c(=O)c2c1]

Combined 8-[(1R)-1-aminoethyl]-2-ethylsulfanyl-3,6-dimethyl-chromen-4-one (0.50 g, 1.80 mmol), zinc(II) 2,6-difluorobenzen-1-ide bromide (0.93 g, 3.61 mmol, 0.5 M), copper(I) thiophene-2-carboxylate (0.51 g, 2.70 mmol), and methanesulfonato{[4-(N,N-dimethylamino)phenyl]di-t-butylphosphino)(2′-amino-1,1′-biphenyl-2-yl)palladium(II) (0.34 g, 0.54 mmol) in THF (5 mL) and degassed for 10 min. The reaction was capped and allowed to stir at 130° C. overnight. The reaction was allowed to cool to rt, diluted with 10 mL of DCM, filtered through celite, and concentrated. The residue was purified by silica gel chromatography eluted with 0% to 100% EtOAc in DCM and then 0% to 20% MeOH in DCM. The desired fractions were then repurified by reversed phase chromatography eluted with 10% to 100% ACN (with 0.1% formic acid) in water (with 0.1% formic acid). The product was then purified by reversed phase chromatography eluted with 10% to 100% ACN in 10 mM NH₄HCO₃ (with 5% MeOH) to give the title compound (0.20 g, 34%). MS ES+ m/z 330 [M+H]⁺.

### Intermediate 104A: tert-Butyl 2-[1-(2-ethylsulfanyl-6-methyl-4-oxo-chromen-8-yl)ethylamino]benzoate

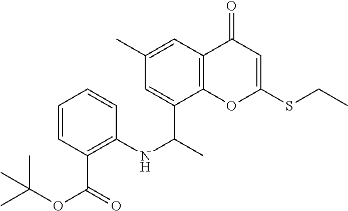 [SMILES: CCSc1cc(=O)c2cc(C)cc(C(C)Nc3ccccc3C(=O)OC(C)(C)C)c2o1]

8-(1-Bromoethyl)-2-ethylsulfanyl-6-methyl-chromen-4-one (25.0 g, 76.4 mmol), tert-butyl 2-aminobenzoate (29.5 g, 153 mmol) and DIEA (14.8 g, 20.0 mL, 115 mmol) were combined with DMF (150 mL) in a 500 mL round bottom flask and heated at 80° C. After cooling to rt, the reaction was partially concentrated to ˜100 mL, poured into 1.1 L of water, and extracted with EtOAc (2×350 mL). The combined organic layers were washed with saturated aqueous sodium chloride (400 mL). The combined aqueous layers were re-extracted with fresh EtOAc. The organic layers were combined, dried over anhydrous Na₂SO₄, filtered, and concentrated to give a thick oil. Purified the residue via silica gel chromatography using EtOAc in DCM (0% to 10%) to provide an off-white foam. Triturated with heptanes/DCM and washed with heptanes to give the product as a white solid (27.1 g, 81%). MS ES+ m/z 440 [M+H]⁺.

### Intermediate 94C: tert-Butyl 2-[1-(2-ethylsulfanyl-6-fluoro-4-oxo-chromen-8-yl)ethylamino]benzoate

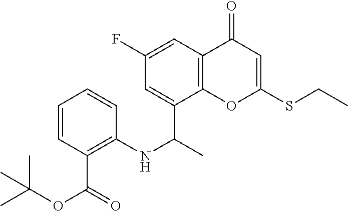 [SMILES: CCSc1cc(=O)c2cc(F)cc(C(C)Nc3ccccc3C(=O)OC(C)(C)C)c2o1]

Dissolved 8-(1-bromoethyl)-2-ethylsulfanyl-6-fluoro-chromen-4-one (66.0 g, 200 mmol) in CHCl₃ (660 mL) and treated with pyridine (17.34 g, 219 mmol) and tert-butyl 2-aminobenzoate (42.36 g, 219 mmol) at rt. The reaction was stirred at 80° C. for 66 h. The reaction was allowed to cool to rt and concentrated under reduced pressure. The crude product was suspended in EtOAc (200 mL), sonicated, and filtered. This procedure was repeated three times. The filtrates were concentrated under reduced pressure and the residue slurried in 300 mL of heptane for 30 min. The solid was removed by filtration, washed with heptane (3×150 mL) and dried to give the title compound (43 g, 49%). ¹HNMR (400 MHz, CDCl₃) δ ppm 1.48˜1.51 (m, 3H), 1.65 (s, 9H), 1.68˜1.70 (d, 3H), 3.11˜3.17 (m, 2H), 5.06˜5.13 (m, 1H), 6.23˜6.26 (d, 1H), 6.33 (s, 1H), 6.59˜6.63 (m, 1H), 7.16˜7.21 (m, 1H), 7.41˜7.44 (m, 1H), 7.69˜7.71 (m, 1H), 7.91˜7.93 (m, 1H), 8.31˜8.32 (m, 1H).

### Intermediate 95C: 6-Chloro-3-[1-[2-ethylsulfanyl-3-methyl-4-oxo-6-(trifluoromethyl)chromen-8-yl]ethylamino]pyridine-2-carboxylic acid

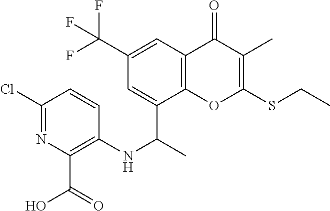 [SMILES: CCSc1oc2c(C(C)Nc3ccc(Cl)nc3C(=O)O)cc(C(F)(F)F)cc2c(=O)c1C]

A solution of 8-[(1R)-1-bromoethyl]-2-ethylsulfanyl-3-methyl-6-(trifluoromethyl)chromen-4-one (223 g, 0.56 mol) and 3-amino-6-chloro-pyridine-2-carboxylic acid (146 g, 0.85 mol) in DMF (1.8 L) was stirred at 85° C. for 3 days. The reaction was cooled to rt and slowly added to water (6.7 L). The resulting precipitate was collected by filtration and the solid slurried in water (3.3 L), filtered, and dried in an oven to give the title compound (232 g, 84%, 73% purity). ES/MS m/z 487 (M+H).

### Intermediate 96C: 6-Chloro-3-[1-(2-ethylsulfanyl-6-fluoro-3-methyl-4-oxo-chromen-8-yl)ethylamino]pyridine-2-carboxylic acid

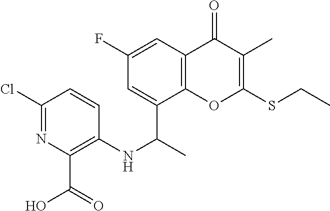 [SMILES: CCSc1oc2c(C(C)Nc3ccc(Cl)nc3C(=O)O)cc(F)cc2c(=O)c1C]

A solution of 8-(1-bromoethyl)-2-ethylsulfanyl-6-fluoro-3-methyl-chromen-4-one (100 g, 290 mmol) in DMF (800 mL) was treated with 3-amino-6-chloro-pyridine-2-carboxylic acid (50 g, 290 mmol). The reaction was stirred at 80 to 90° C. for 58 h. The reaction was concentrated under reduced pressure at 60° C. and the residue slurried with MTBE (500 mL) and water (100 mL). After 30 min under sonication, the reaction was filtered and the solids washed with MTBE (2×500 mL) to give the title compound (64 g, 51%) as a light yellow solid. ¹HNMR (400 MHz, CDCl₃) δ ppm 1.47˜1.51 (m, 3H), 1.64˜1.66 (m, 3H), 1.98 (s, 3H) 3.09˜ 3.36 (m, 2H), 5.15˜5.21 (m, 1H), 7.00˜7.02 (d, 1H), 7.30˜7.35 (m, 1H), 7.49˜7.51 (m, 1H), 7.58˜7.61 (m, 1H) 8.29˜8.31 (m, 1H), 13.15 (br, 1H).

### Intermediate 97C: tert-Butyl 6-chloro-3-[1-(2-ethylsulfanyl-6-fluoro-3-methyl-4-oxo-chromen-8-yl)ethylamino]pyridine-2-carboxylate

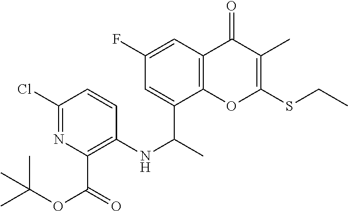 [SMILES: CCSc1oc2c(C(C)Nc3ccc(Cl)nc3C(=O)OC(C)(C)C)cc(F)cc2c(=O)c1C]

A solution of 6-chloro-3-[1-(2-ethylsulfanyl-6-fluoro-3-methyl-4-oxo-chromen-8-yl)ethylamino]pyridine-2-carboxylic acid (79 g, 181 mmol) in THF (800 mL) was cooled to 10° C. and treated dropwise with 2-tert-butyl-1,3-diisopropyl-isourea (126.78 g, 633 mmol). The reaction was stirred at 20° C. for 2 h, filtered, and the solids washed with THF (2×100 mL). The filtrate was concentrated under reduced pressure and the residue was slurried in 200 mL of MTBE under sonication for 30 min giving the title compound (76.3 g, 86%) after filtration, washing with MTBE (100 mL) and drying. ¹HNMR (400 MHz, CDCl₃) δ ppm 1.47˜1.51 (m, 3H), 1.63˜1.74 (m, 12H), 2.12 (s, 3H), 3.21˜ 3.26 (m, 2H), 5.01˜5.07 (m, 1H), 6.59˜6.61 (d, 1H), 7.09˜7.11 (d, 1H), 7.30˜7.33 (m, 1H), 7.75˜7.78 (m, 1H), 8.32˜8.33 (d, 1H).

The following compound in Table 15 was made in a similar way as described for tert-butyl 6-chloro-3-[1-(2-ethylsulfanyl-6-fluoro-3-methyl-4-oxo-chromen-8-yl)ethylamino]pyridine-2-carboxylate. Various methods were used to purify the compound, which would be apparent to one skilled in the art.

TABLE 15

| | | | MS ES+ |
|---|---|---|---|
| Interm # | Chemical Name | Structure | m/z |
| 98C | tert-Butyl 6-chloro-3- [1-[2-ethylsulfanyl-3- methyl-4-oxo-6- (trifluoromethyl)chromen- 8- yl]ethylamino]pyridine- 2-carboxylate, Isomer 1 and Isomer 2 | 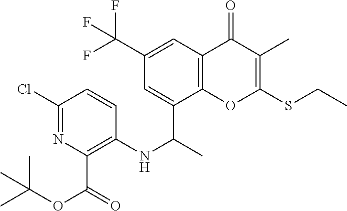 [SMILES: CCSc1oc2c(C(C)Nc3ccc(Cl)nc3C(=O)OC(C)(C)C)cc(C(F)(F)F)cc2c(=O)c1C] | 543 [M + H]⁺ |

### Intermediate 99C: Methyl 4-[[(1R)-1-[3,6-dimethyl-2-(2-methylindazol-5-yl)-4-oxo-chromen-8-yl]ethyl]amino]-6-methyl-2-oxo-1H-pyridine-3-carboxylate

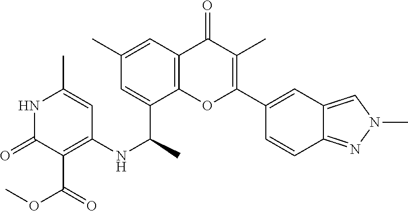 [SMILES: COC(=O)c1c(N[C@H](C)c2cc(C)cc3c(=O)c(C)c(-c4ccc5nn(C)cc5c4)oc23)cc(C)[nH]c1=O]

Combined 8-[(1R)-1-aminoethyl]-3,6-dimethyl-2-(2-methylindazol-5-yl)chromen-4-one (100 mg, 0.29 mmol), methyl 4-chloro-6-methyl-2-oxo-1H-pyridine-3-carboxylate (87 mg, 0.43 mmol), and triethylamine (58.3 mg, 0.58 mmol) in MeOH (1 mL) and stirred the reaction at 75° C. for 60 h. Cooled the reaction to rt and concentrated under reduced pressure to give the title compound (148 mg, 100%). ES/MS m/z 513 (M+H).

The following compound in Table 16 was made in a similar way as described for methyl 4-[[(1R)-1-[3,6-dimethyl-2-(2-methylindazol-5-yl)-4-oxo-chromen-8-yl]ethyl]amino]-6-methyl-2-oxo-1H-pyridine-3-carboxylate. Various methods were used to purify the compound, which would be apparent to one skilled in the art.

TABLE 16

| | | | MS ES+ |
|---|---|---|---|
| Interm # | Chemical Name | Structure | m/z |
| 100C | Methyl 4-[(1R)-1-(3,6- dimethyl-4-oxo-2-phenyl- chromen-8-yl)ethyl]amino]-6- methyl-2-oxo-1H-pyridine-3- carboxylate | 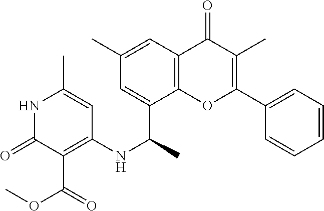 [SMILES: COC(=O)c1c(N[C@H](C)c2cc(C)cc3c(=O)c(C)c(-c4ccccc4)oc23)cc(C)[nH]c1=O] | 459 [M + H]⁺ |

### Intermediate 105A and Intermediate 106A: tert-Butyl 2-[1-(2-ethylsulfanyl-6-methyl-4-oxo-chromen-8-yl)ethylamino]benzoate, Isomer 1 and Isomer 2

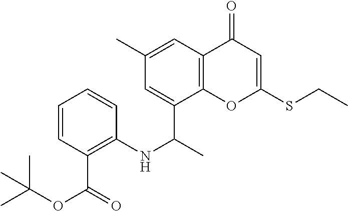 [SMILES: CCSc1cc(=O)c2cc(C)cc(C(C)Nc3ccccc3C(=O)OC(C)(C)C)c2o1]

tert-Butyl 2-[1-(2-ethylsulfanyl-6-methyl-4-oxo-chromen-8-yl)ethylamino]benzoate (22.04 g, 50.14 mmol) was separated into component isomers using a Chiralcel OJ column (8×34 cm; 20 micron) eluted with 100% MeOH with 0.2% DMEA to give isomer 1 (wet 11.3 g) and isomer 2 (wet 12.9 g). MS ES+ m/z 440 [M+H]⁺.

### Intermediate 101C and Intermediate 102C: tert-Butyl 6-chloro-3-[1-(2-ethylsulfanyl-6-fluoro-3-methyl-4-oxo-chromen-8-yl)ethylamino]pyridine-2-carboxylate, Isomer 1 and Isomer 2

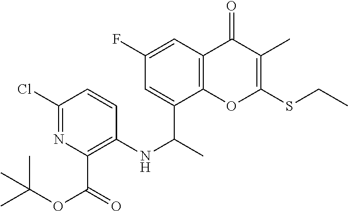 [SMILES: CCSc1oc2c(C(C)Nc3ccc(Cl)nc3C(=O)OC(C)(C)C)cc(F)cc2c(=O)c1C]

tert-Butyl 6-chloro-3-[1-(2-ethylsulfanyl-6-fluoro-3-methyl-4-oxo-chromen-8-yl)ethylamino]pyridine-2-carboxylate (76.3 g, 155 mmol) was separated into component isomers using a ChiralPak IC column (250×50 mm, 10 μm) eluted with 45% i-PrOH (with 0.1% NH₄OH) in CO₂ to give crude Isomer 1 (38.40 g) and crude Isomer 2 (27 g). Isomer 1 was repurified by preparative HPLC using a Sharpsil-T C18 column (50×250 mm, 8 microns) eluted with 85% to 98% ACN in water (with 0.05% TFA) to give Isomer 1 (28.60 g, 37%) as an off-white solid. ES/MS m/z 493 (M+H).

### Intermediate 103C and Intermediate 104C. tert-Butyl 2-[1-(2-ethylsulfanyl-6-fluoro-4-oxo-chromen-8-yl)ethylamino]benzoate, Isomer 1 and Isomer 2

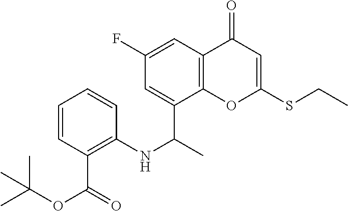 [SMILES: CCSc1cc(=O)c2cc(F)cc(C(C)Nc3ccccc3C(=O)OC(C)(C)C)c2o1]

tert-Butyl 2-[1-(2-ethylsulfanyl-6-fluoro-4-oxo-chromen-8-yl)ethylamino]benzoate (48.3 g, 109 mmol) was separated into component isomers using chiral SFC [ChiralPak IG, 250×50 mm, 10 μm; MeOH in CO₂] to give Isomer 1 (22.4 g, 46%) and Isomer 2 (19.0 g, 39%) as yellow solids. ES/MS m/z 466 (M+H).

### Intermediate 105C and Intermediate 106C: tert-Butyl 6-chloro-3-[1-[2-ethylsulfanyl-3-methyl-4-oxo-6-(trifluoromethyl)chromen-8-yl]ethylamino]pyridine-2-carboxylate, Isomer 1 and Isomer 2

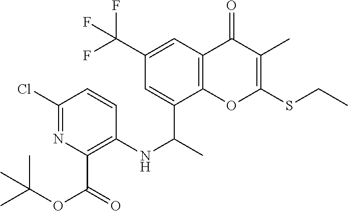 [SMILES: CCSc1oc2c(C(C)Nc3ccc(Cl)nc3C(=O)OC(C)(C)C)cc(C(F)(F)F)cc2c(=O)c1C]

tert-Butyl 6-chloro-3-[1-[2-ethylsulfanyl-3-methyl-4-oxo-6-(trifluoromethyl)chromen-8-yl]ethylamino]pyridine-2-carboxylate was separated into component isomers using chiral SFC [WhelkO1-(R,R), 50×250 mm, 10 μm; 25% EtOH in CO₂] to give Isomer 1 (first eluting; 67.7 g, 99% ee) and Isomer 2 (68.0 g, 93.6% ee). ES/MS m/z 543 (M+H).

### Intermediate 107A: tert-Butyl 6-chloro-3-[[(1R)-1-(2-ethylsulfanyl-3,6-dimethyl-4-oxo-chromen-8-yl)ethyl]amino]pyridine-2-carboxylate

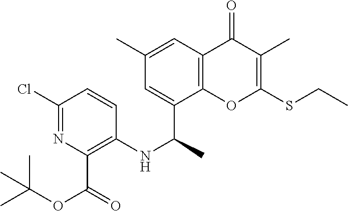 [SMILES: CCSc1oc2c([C@@H](C)Nc3ccc(Cl)nc3C(=O)OC(C)(C)C)cc(C)cc2c(=O)c1C]

A vial was charged with 8-[(1R)-1-aminoethyl]-2-ethylsulfanyl-3,6-dimethyl-chromen-4-one (1.0 g, 3.61 mmol), tert-butyl 3-bromo-6-chloropicolinate (1.58 g, 5.41 mmol), tris(dibenzylideneacetone)dipalladium(0) (0.25 g, 0.27 mmol), 4,5-bis(diphenylphosphino)-9,9-dimethylxanthene (0.42 g, 0.72 mmol), cesium carbonate (2.93 g, 9.01 mmol), and 12 mL of toluene. The suspension was purged with nitrogen gas for 10 min and the vial sealed. The reaction was stirred at 130° C. overnight. The reaction was cooled to rt, filtered, and the solids washed with DCM. The filtrate was concentrated and the residue purified by silica gel chromatography eluted with 0% to 100% EtOAc in hexane to give the title compound (0.88 g, 50%) as a pale tan solid. MS ES− m/z 433 [M+2H−tBu]⁺.

The following compounds in Table 17 were made in a similar way as described for tert-butyl 6-chloro-3-[[(1R)-1-(2-ethylsulfanyl-3,6-dimethyl-4-oxo-chromen-8-yl)ethyl]amino]pyridine-2-carboxylate. Various methods were used to purify the compounds, which would be apparent to one skilled in the art.

TABLE 17

| | | | MS ES+ |
|---|---|---|---|
| Interm # | Chemical Name | Structure | m/z |
| 108A | 8-[(1R)-1-[(2-Chloro- 3-pyridyl)amino]ethyl]- 2-ethylsulfanyl-3,6- dimethyl-chromen-4- one | 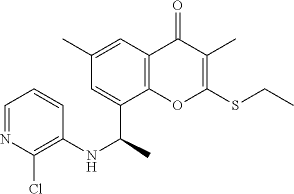 [SMILES: CCSc1oc2c([C@@H](C)Nc3cccnc3Cl)cc(C)cc2c(=O)c1C] | 389 [M + H]⁺ |
| 109A | 2-[[(1R)-1-(2- Ethylsulfanyl-3,6- dimethyl-4-oxo- chromen-8- yl)ethyl]amino]-4- fluoro-benzonitrile | 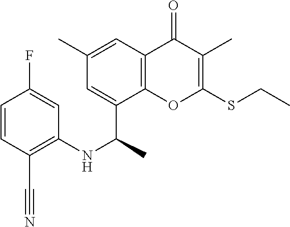 [SMILES: CCSc1oc2c([C@@H](C)Nc3cc(F)ccc3C#N)cc(C)cc2c(=O)c1C] | 397 [M + H]⁺ |
| 110A | tert-Butyl 3-[[(1R)-1- [2-[6-(difluoromethyl)- 2-pyridyl]-3,6- dimethyl-4-oxo- chromen-8- yl]ethyl]amino]pyridine- 2-carboxylate | 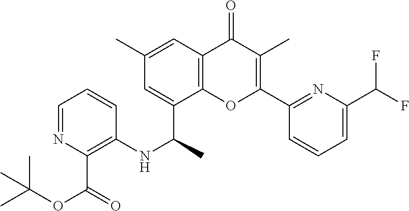 [SMILES: Cc1cc([C@@H](C)Nc2cccnc2C(=O)OC(C)(C)C)c2oc(-c3cccc(C(F)F)n3)c(C)c(=O)c2c1] | 522 [M + H]⁺ |
| 111A | N′-[2-[(1R)-1-(3,6- Dimethyl-4-oxo-2- phenyl-chromen-8- yl)ethyl]amino]phenyl] sulfonyl-N,N-dimethyl- formamidine | 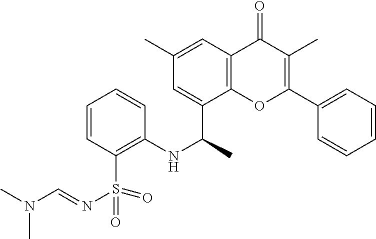 [SMILES: Cc1cc([C@@H](C)Nc2ccccc2S(=O)(=O)/N=C/N(C)C)c2oc(-c3ccccc3)c(C)c(=O)c2c1] | 504 [M + H]⁺ |
| 112A | tert-Butyl 2-[[(1R)-1- [3,6-dimethyl-4-oxo-2- (3-pyridyl)chromen-8- yl]ethyl]amino]benzoate | 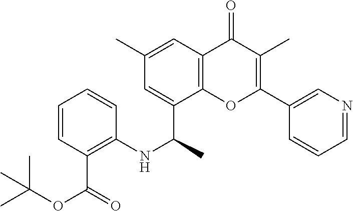 [SMILES: Cc1cc([C@@H](C)Nc2ccccc2C(=O)OC(C)(C)C)c2oc(-c3cccnc3)c(C)c(=O)c2c1] | 471 [M + H]⁺ |
| 113A | tert-butyl 6-chloro-3- [[(1R)-1-[3,6-dimethyl- 4-oxo-2-(3- pyridyl)chromen-8- yl]ethyl]amino]pyridine- 2-carboxylate | 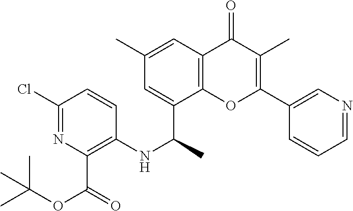 [SMILES: Cc1cc([C@@H](C)Nc2ccc(Cl)nc2C(=O)OC(C)(C)C)c2oc(-c3cccnc3)c(C)c(=O)c2c1] | 450 [M + H − tBu]⁺ |
| 114A¹ | tert-Butyl 2-[[(1R)-1- (2-ethylsulfanyl-3,6- dimethyl-4-oxo- chromen-8- yl)ethyl]amino]benzoate | 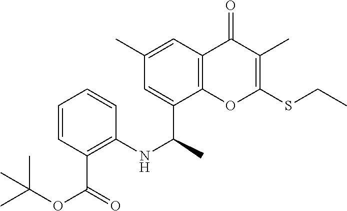 [SMILES: CCSc1oc2c([C@@H](C)Nc3ccccc3C(=O)OC(C)(C)C)cc(C)cc2c(=O)c1C] | |
| 115A | tert-Butyl 3-[[(1R)-1- [3,6-dimethyl-2-(2- methylindazol-5-yl)-4- oxo-chromen-8- yl]ethyl]amino]pyridine- 2-carboxylate | 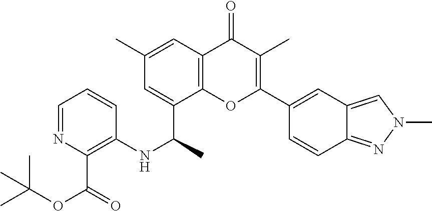 [SMILES: Cc1cc([C@@H](C)Nc2cccnc2C(=O)OC(C)(C)C)c2oc(-c3ccc4nn(C)cc4c3)c(C)c(=O)c2c1] | 525 [M + H]⁺ |
| 116A | tert-Butyl 3-[[(1R)-1- [3,6-dimethyl-4-oxo-2- (3-pyridyl)chromen-8- yl]ethyl]amino]-6- fluoro-pyridine-2- carboxylate | 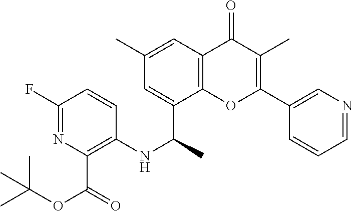 [SMILES: Cc1cc([C@@H](C)Nc2ccc(F)nc2C(=O)OC(C)(C)C)c2oc(-c3cccnc3)c(C)c(=O)c2c1] | 490 [M + H]⁺ |
| 117A | tert-Butyl 6-chloro-3- [(1R)-1-(2- ethylsulfanyl-6-methyl- 4-oxo-chromen-8- yl)ethyl]amino]pyridine- 2-carboxylate | 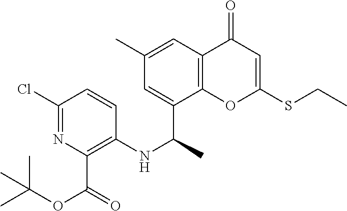 [SMILES: CCSc1cc(=O)c2cc(C)cc([C@@H](C)Nc3ccc(Cl)nc3C(=O)OC(C)(C)C)c2o1] | 475 [M + H]⁺ |
| 107C | tert-Butyl 6-[[(1R)-1- [3,6-dimethyl-4-oxo-2- (3-pyridyl)chromen-8- yl]ethyl]amino]-2,3- difluoro-benzoate | 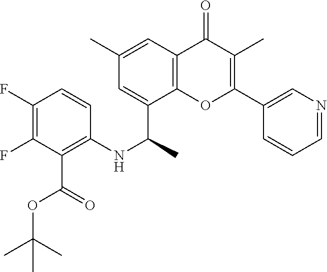 [SMILES: Cc1cc([C@@H](C)Nc2ccc(F)c(F)c2C(=O)OC(C)(C)C)c2oc(-c3cccnc3)c(C)c(=O)c2c1] | 507 [M + H]⁺ |
| 108C | tert-Butyl 4-[6-chloro- 3-[[(1R)-1-[3,6- dimethyl-4-oxo-2-(3- pyridyl)chromen-8- yl]ethyl]amino]-2- pyridyl]piperazine-1- carboxylate | 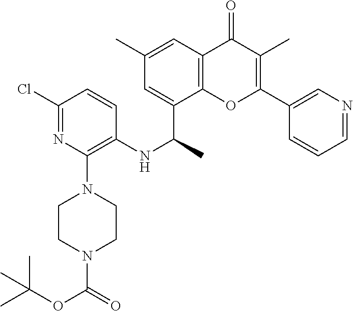 [SMILES: Cc1cc([C@@H](C)Nc2ccc(Cl)nc2N2CCN(C(=O)OC(C)(C)C)CC2)c2oc(-c3cccnc3)c(C)c(=O)c2c1] | 590 [M + H]⁺ |
| 109C | 8-[(1R)-1-[(6-Chloro- 2-fluoro-3- pyridyl)amino]ethyl]- 3,6-dimethyl-2-(3- pyridyl)chromen-4-one | 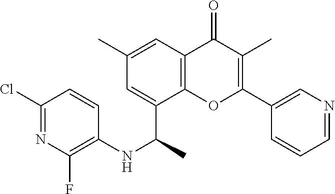 [SMILES: Cc1cc([C@@H](C)Nc2ccc(Cl)nc2F)c2oc(-c3cccnc3)c(C)c(=O)c2c1] | 424 [M + H]⁺ |
| 110C | tert-Butyl 6-chloro-3- [[(1R)-1-[3,6-dimethyl- 2-[1-(oxetan-3- yl)pyrazol-4-yl]-4-oxo- chromen-8- yl]ethyl]amino]pyridine- 2-carboxylate | 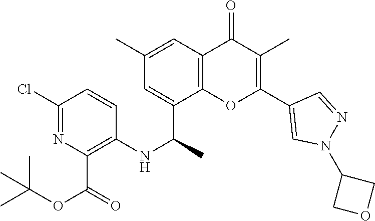 [SMILES: Cc1cc([C@@H](C)Nc2ccc(Cl)nc2C(=O)OC(C)(C)C)c2oc(-c3cnn(C4COC4)c3)c(C)c(=O)c2c1] | 495 [MH − ^{t}Bu]⁺ |
| 111C | tert-Butyl 6-[[(1R)-1- [3,6-dimethyl-2-(1- methylpyrazol-4-yl)-4- oxo-chromen-8- yl]ethyl]amino]-2,3- difluoro-benzoate | 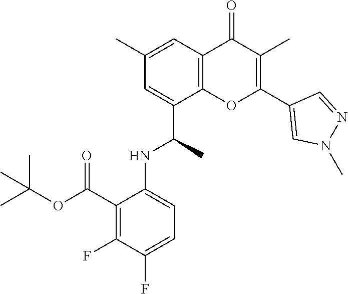 [SMILES: Cc1cc([C@@H](C)Nc2ccc(F)c(F)c2C(=O)OC(C)(C)C)c2oc(-c3cnn(C)c3)c(C)c(=O)c2c1] | 510 [M + H]⁺ |
| 112C | 6-Chloro-3-[(1R)-1- (2-ethylsulfanyl-3,6- dimethyl-4-oxo- chromen-8- yl)ethyl]amino]pyridine- 2-carbonitrile | 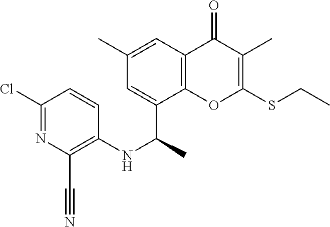 [SMILES: CCSc1oc2c([C@@H](C)Nc3ccc(Cl)nc3C#N)cc(C)cc2c(=O)c1C] | 414 [M + H]⁺ |
| 113C | 6-Chloro-3-[[(1R)-1- [2-[1-(2- methoxyethyl)pyrazol- 4-yl]-3,6-dimethyl-4- oxo-chromen-8- yl]ethyl]amino]pyridine- 2-carbonitrile | 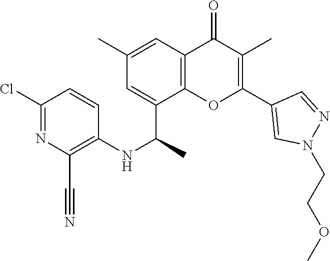 [SMILES: COCCn1cc(-c2oc3c([C@@H](C)Nc4ccc(Cl)nc4C#N)cc(C)cc3c(=O)c2C)cn1] | 478 [M + H]⁺ |
| 114C² | tert-Butyl 3-[[(1R)-1- (2-ethylsulfanyl-3,6- dimethyl-4-oxo- chromen-8- yl)ethyl]amino]-6- fluoro-pyridine-2- carboxylate | 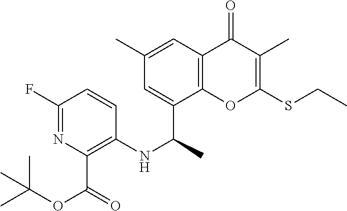 [SMILES: CCSc1oc2c([C@@H](C)Nc3ccc(F)nc3C(=O)OC(C)(C)C)cc(C)cc2c(=O)c1C] | 473 [M + H]⁺ |
| 115C | 6-Chloro-3-[(1R)-1- [2-(1,5- dimethylpyrazol-4-yl)- 3,6-dimethyl-4-oxo- chromen-8- yl]ethyl]amino]pyridine- 2-carbonitrile | 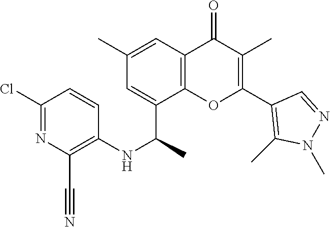 [SMILES: Cc1cc([C@@H](C)Nc2ccc(Cl)nc2C#N)c2oc(-c3cnn(C)c3C)c(C)c(=O)c2c1] | 448 [M + H]⁺ |
| 116C | 6-Chloro-3-[[(1R)-1- [2-[1-(1-cyano-1- methyl-ethyl)pyrazol- 4-yl]-3,6-dimethyl-4- oxo-chromen-8- yl]ethyl]amino]pyridine- 2-carbonitrile | 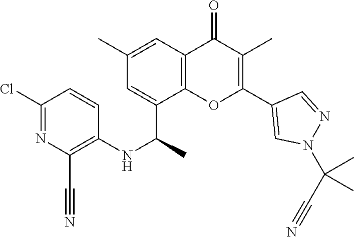 [SMILES: Cc1cc([C@@H](C)Nc2ccc(Cl)nc2C#N)c2oc(-c3cnn(C(C)(C)C#N)c3)c(C)c(=O)c2c1] | 487 [M + H]⁺ |
| 117C | Methyl 2-[(1R)-1-[3,6- dimethyl-2-(2- methylindazol-5-yl)-4- oxo-chromen-8- yl]ethyl]amino]-6- fluoro-benzoate | 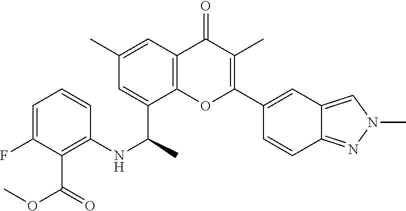 | 500 [M + H]⁺ |
| 118C | 6-Chloro-3-[[(1R)-1- [3,6-dimethyl-2-(2- methylpyrazol-3-yl)-4- oxo-chromen-8- yl]ethyl]amino]pyridine- 2-carbonitrile | 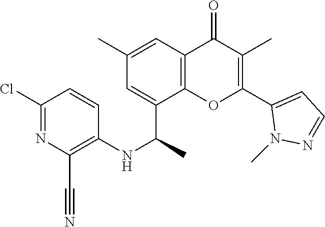 [SMILES: Cc1cc([C@@H](C)Nc2ccc(Cl)nc2C#N)c2oc(-c3ccnn3C)c(C)c(=O)c2c1] | 434 [M + H]⁺ |
| 119C | 6-Chloro-3-[(1R)-1- [2-[1- (difluoromethyl)pyrazo1- 4-yl]-3,6-dimethyl-4- oxo-chromen-8- yl]ethyl]amino]pyridine- 2-carbonitrile | 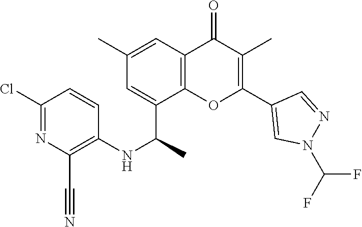 [SMILES: Cc1cc([C@@H](C)Nc2ccc(Cl)nc2C#N)c2oc(-c3cnn(C(F)F)c3)c(C)c(=O)c2c1] | 470 [M + H]⁺ |
| 120C | 6-Chloro-3-[(1R)-1- [2-(1-isopropylpyrazol- 4-yl)-3,6-dimethyl-4- oxo-chromen-8- yl]ethyl]amino]pyridine- 2-carbonitrile | 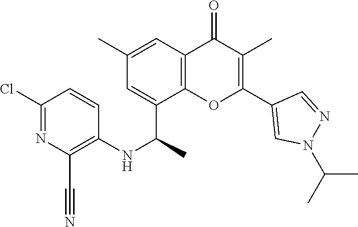 [SMILES: Cc1cc([C@@H](C)Nc2ccc(Cl)nc2C#N)c2oc(-c3cnn(C(C)C)c3)c(C)c(=O)c2c1] | 462 [M + H]⁺ |
| 121C | 6-Chloro-3-[(1R)-1- [2-(1- cyclopropylpyrazol-4- yl)-3,6-dimethyl-4-oxo- chromen-8- yl]ethyl]amino]pyridine- 2-carbonitrile | 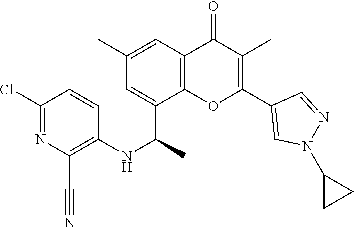 [SMILES: Cc1cc([C@@H](C)Nc2ccc(Cl)nc2C#N)c2oc(-c3cnn(C4CC4)c3)c(C)c(=O)c2c1] | 460 [M + H]⁺ |
| 122C | 6-Chloro-3-[[(1R)-1- (3,6-dimethyl-4-oxo-2- pyrimidin-5-yl- chromen-8- yl)ethyl]amino]pyridine- 2-carbonitrile | 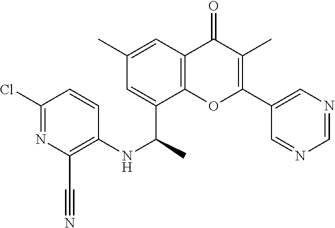 [SMILES: Cc1cc([C@@H](C)Nc2ccc(Cl)nc2C#N)c2oc(-c3cncnc3)c(C)c(=O)c2c1] | 432 [M + H]⁺ |
| 123C | 6-Chloro-3-[(1R)-1- [3,6-dimethyl-4-oxo-2- [1- (trideuteriomethyl)pyrazol- 4-yl]chromen-8- yl]ethyl]amino]pyridine- 2-carbonitrile | 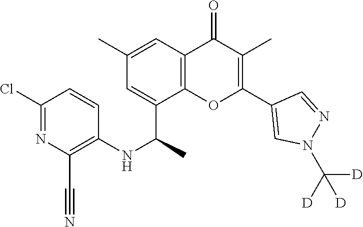 [SMILES: [2H]C([2H])([2H])n1cc(-c2oc3c([C@@H](C)Nc4ccc(Cl)nc4C#N)cc(C)cc3c(=O)c2C)cn1] | 437 [M + H]⁺ |
| 124C | 6-Chloro-3-[[(1R)-1- deuterio-1-(3,6- dimethyl-4-oxo-2- phenyl-chromen-8- yl)ethyl]amino]pyridine- 2-carbonitrile | 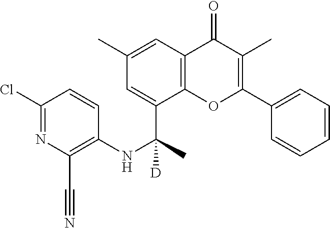 [SMILES: [2H][C@](C)(Nc1ccc(Cl)nc1C#N)c1cc(C)cc2c(=O)c(C)c(-c3ccccc3)oc12] | 431 [M + H]⁺ |
| 125C | 3-[[(1R)-1-Deuterio-1- (3,6-dimethyl-4-oxo-2- phenyl-chromen-8- yl)ethyl]amino]pyridine- 2-carbonitrile | 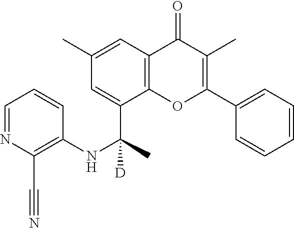 [SMILES: [2H][C@](C)(Nc1cccnc1C#N)c1cc(C)cc2c(=O)c(C)c(-c3ccccc3)oc12] | 397 [M + H]⁺ |
| 126C | 6-Chloro-3-[[(1R)-1- (2-ethylsulfanyl-6- methyl-4-oxo- chromen-8- yl)ethyl]amino]pyridine- 2-carbonitrile | 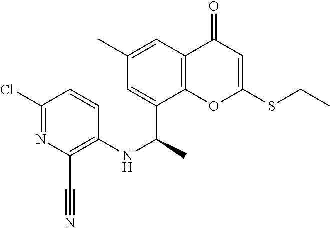 [SMILES: CCSc1cc(=O)c2cc(C)cc([C@@H](C)Nc3ccc(Cl)nc3C#N)c2o1] | 400 [M + H]⁺ |
| 127C | 8-[(1R)-1-[2-(1,3- Dioxolan-2- yl)anilino]ethyl]-3,6- dimethyl-2-(3- pyridyl)chromen-4-one | 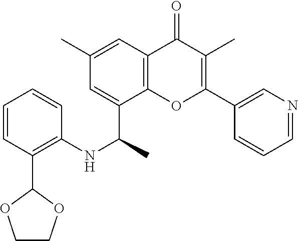 [SMILES: Cc1cc([C@@H](C)Nc2ccccc2C2OCCO2)c2oc(-c3cccnc3)c(C)c(=O)c2c1] | 443 [M + H]⁺ |
| 128C | tert-Butyl 6-chloro-3- [[(1R)-1-[3,6-dimethyl- 2-(1-methylindazol-5- yl)-4-oxo-chromen-8- yl]ethyl]amino]pyridine- 2-carboxylate | 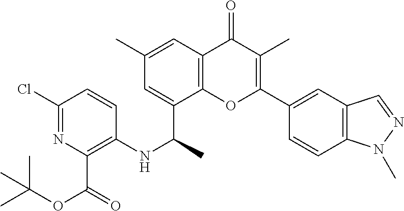 [SMILES: Cc1cc([C@@H](C)Nc2ccc(Cl)nc2C(=O)OC(C)(C)C)c2oc(-c3ccc4c(cnn4C)c3)c(C)c(=O)c2c1] | 503 [MH − tBu]⁺ |
| 129C² | 8-[(1R)-1-[(6-Chloro- 2-methyl-3- pyridyl)amino]ethyl]-2- ethylsulfanyl-6-methyl- chromen-4-one | 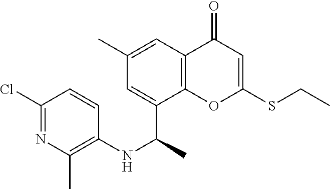 [SMILES: CCSc1cc(=O)c2cc(C)cc([C@@H](C)Nc3ccc(Cl)nc3C)c2o1] | 389 [M + H]⁺ |
| 130C | 2-Fluoro-6-[[(1R)-1-[2- (5-fluoro-3-pyridyl)- 3,6-dimethyl-4-oxo- chromen-8- yl]ethyl]amino] benzonitrile | 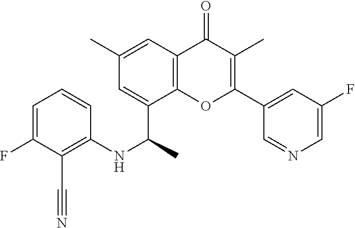 [SMILES: Cc1cc([C@@H](C)Nc2cccc(F)c2C#N)c2oc(-c3cncc(F)c3)c(C)c(=O)c2c1] | 432 [M + H]⁺ |
| 131C | 6-[[(1R)-1-[3,6- Dimethyl-4-oxo-2-(2- pyridyl)chromen-8- yl]ethyl]amino]-2,3- difluoro-benzonitrile | 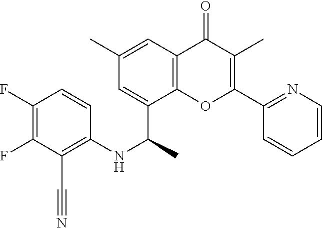 [SMILES: Cc1cc([C@@H](C)Nc2ccc(F)c(F)c2C#N)c2oc(-c3ccccn3)c(C)c(=O)c2c1] | 432 [M + H]⁺ |
| 132C | 2-Ethylsulfanyl-3,6- dimethyl-8-[(1R)-1-[[6- (trifluoromethyl)-3- pyridyl]amino]ethyl] chromen-4-one | 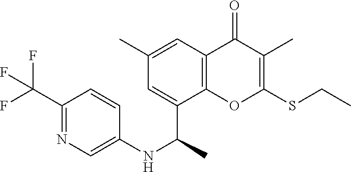 [SMILES: CCSc1oc2c([C@@H](C)Nc3ccc(C(F)(F)F)nc3)cc(C)cc2c(=O)c1C] | 423 [M + H]⁺ |
| 133C | 8-[(1R)-1-[(2-Bromo- 6-chloro-3- pyridyl)amino]ethyl]- 3,6-dimethyl-2-(3- pyridyl)chromen-4-one | 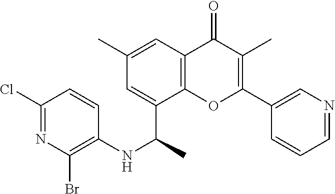 [SMILES: Cc1cc([C@@H](C)Nc2ccc(Cl)nc2Br)c2oc(-c3cccnc3)c(C)c(=O)c2c1] | 484/486 [M + H]⁺ |
| 134C | tert-Butyl 3-[8-[(1R)-1- [(2-tert- butoxycarbonyl-6- chloro-3- pyridyl)amino]ethyl]- 3,6-dimethyl-4-oxo- chromen-2-yl]-6,7- dihydro-4H- pyrazolo[1,5- a]pyrazine-5- carboxylate | 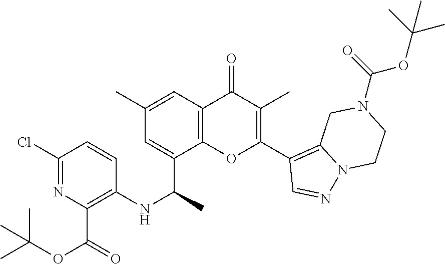 [SMILES: Cc1cc([C@@H](C)Nc2ccc(Cl)nc2C(=O)OC(C)(C)C)c2oc(-c3cnn4c3CN(C(=O)OC(C)(C)C)CC4)c(C)c(=O)c2c1] | 594 [M − ^{t}Bu]⁺ |
| 135C | 8-[(1R)-1-[[2-[3-[tert- Butyl(dimethyl)silyl] oxyazetidin-1-yl]-6- chloro-3- pyridyl]amino]ethyl]- 3,6-dimethyl-2-(3- pyridyl)chromen-4-one | 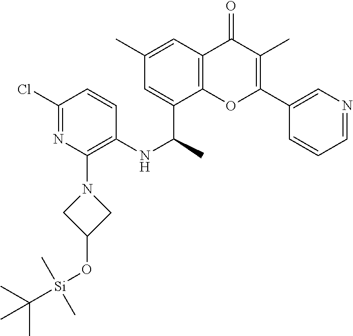 [SMILES: Cc1cc([C@@H](C)Nc2ccc(Cl)nc2N2CC(O[Si](C)(C)C(C)(C)C)C2)c2oc(-c3cccnc3)c(C)c(=O)c2c1] | 591 [M + H]⁺ |
| 136C | 6-Chloro-3-[[(1R)-1- (3,6-dimethyl-4-oxo-2- pyrazolo[1,5- a]pyrimidin-3-yl- chromen-8- yl)ethyl]amino]pyridine- 2-carbonitrile | 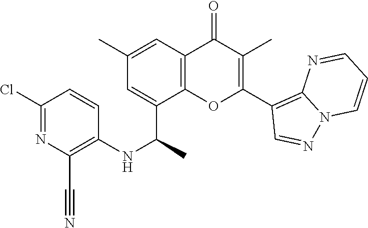 [SMILES: Cc1cc([C@@H](C)Nc2ccc(Cl)nc2C#N)c2oc(-c3cnn4cccnc34)c(C)c(=O)c2c1] | 471 [M + H]⁺ |
| 137C | tert-Butyl 6-chloro-3- [[(1R)-1-(3,6-dimethyl- 4-oxo-2-pyrazolo[1,5- a]pyrimidin-3-yl- chromen-8- yl)ethyl]amino]pyridine- 2-carboxylate | 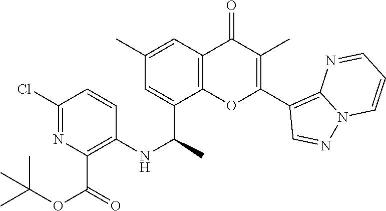 [SMILES: Cc1cc([C@@H](C)Nc2ccc(Cl)nc2C(=O)OC(C)(C)C)c2oc(-c3cnn4cccnc34)c(C)c(=O)c2c1] | 490 [MH − ^{t}Bu]⁺ |
| 138C | Methyl 6-chloro-3- [[(1R)-1-(2- ethylsulfanyl-3,6- dimethyl-4-oxo- chromen-8- yl)ethyl]amino]pyridine- 2-carboxylate | 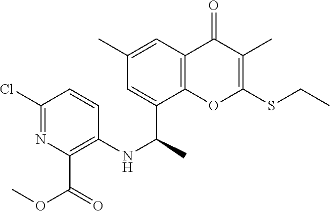 [SMILES: CCSc1oc2c([C@@H](C)Nc3ccc(Cl)nc3C(=O)OC)cc(C)cc2c(=O)c1C] | 447 [M + H]⁺ |

¹1,4-Dioxane used as solvent.
²XantPhos Pd G4 used instead of Pd₂(dba)₃ and Xantphos.

### Intermediate 139C: N-tert-Butyl-6-chloro-3-[[(1R)-1-(2-ethylsulfanyl-3,6-dimethyl-4-oxo-chromen-8-yl)ethyl]amino]pyridine-2-sulfonamide

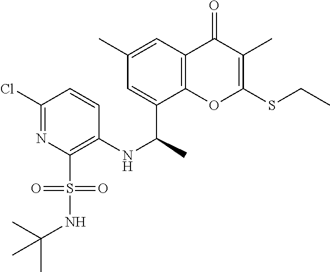 [SMILES: CCSc1oc2c([C@@H](C)Nc3ccc(Cl)nc3S(=O)(=O)NC(C)(C)C)cc(C)cc2c(=O)c1C]

A solution of 8-[(1R)-1-aminoethyl]-2-ethylsulfanyl-3,6-dimethyl-chromen-4-one (10 g, 36 mmol) in DMF (150 mL) was treated with cesium carbonate (23.49 g, 72 mmol), 3-bromo-N-tert-butyl-6-chloro-pyridine-2-sulfonamide (15.36 g, 47.0 mmol), copper(I) iodide (1.37 g, 7 mmol), and N,N′-bis(furan-2-ylmethyl)oxalamide (1.79 g, 7 mmol) at rt. The reaction was stirred at 100° C. for 12 h. The reaction was allowed to cool to rt and filtered. The filtrate was concentrated under reduced pressure and the residue purified by silica gel chromatography eluted with 17% EtOAc in heptane to give the title compound (12.3 g, 65%) as an off-white solid. ES/MS m/z 524 (M+H).

### Intermediate 140C: tert-Butyl 6-chloro-3-[[(1R)-1-(2-ethylsulfinyl-3,6-dimethyl-4-oxo-chromen-8-yl)ethyl]amino]pyridine-2-carboxylate

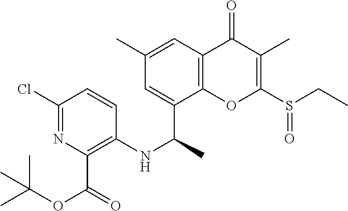 [SMILES: CCS(=O)c1oc2c([C@@H](C)Nc3ccc(Cl)nc3C(=O)OC(C)(C)C)cc(C)cc2c(=O)c1C]

A solution of tert-butyl 6-chloro-3-[[(1R)-1-(2-ethylsulfanyl-3,6-dimethyl-4-oxo-chromen-8-yl)ethyl]amino]pyridine-2-carboxylate (1.0 g, 2.05 mmol) in DCM (10 mL) was cooled to 0° C. and treated in portions with mCPBA (0.50 g, 77%, 2.25 mmol). After addition was complete, the reaction was stirred at 0° C. for 1 h. The reaction was diluted with DCM and washed with saturated aqueous NaHCO₃. The organic layer was removed and the aqueous layer extracted with DCM. The combined organic layers were dried over MgSO₄, filtered, and concentrated under reduced pressure. The residue was purified by silica gel chromatography eluted with 0% to 100% EtOAc in heptane to give the title compound (1.0 g, 97%) as a pale tan solid. ES/MS m/z 503 (M−H).

The following compounds in Table 18 were made in a similar way as described for tert-butyl 6-chloro-3-[[(1R)-1-(2-ethylsulfinyl-3,6-dimethyl-4-oxo-chromen-8-yl)ethyl]amino]pyridine-2-carboxylate. Various methods were used to purify the compounds, which would be apparent to one skilled in the art.

TABLE 18

| | | | MS ES+ |
|---|---|---|---|
| Interm # | Chemical Name | Structure | m/z |
| 141C | Methyl 6-chloro-3-[(1R)- 1-(2-ethyl-sulfinyl-3,6- dimethyl-4-oxo-chromen- 8-yl)ethyl]amino]pyridine- 2-carboxylate | 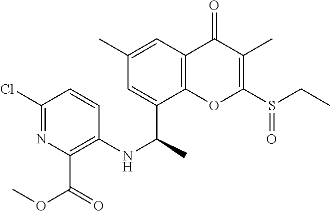 [SMILES: CCS(=O)c1oc2c([C@@H](C)Nc3ccc(Cl)nc3C(=O)OC)cc(C)cc2c(=O)c1C] | 463 [M + H]⁺ |
| 142C | N-tert-Butyl-6-chloro-3- [[(1R)-1-(2-ethylsulfinyl- 3,6-dimethyl-4-oxo-chro- men-8-yl)ethyl]amino]- pyridine-2-sulfonamide | 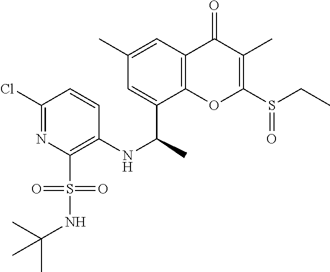 [SMILES: CCS(=O)c1oc2c([C@@H](C)Nc3ccc(Cl)nc3S(=O)(=O)NC(C)(C)C)cc(C)cc2c(=O)c1C] | 540 [M + H]⁺ |

### Intermediate 143C: tert-Butyl 6-chloro-3-[[(1R)-1-(2-chloro-3,6-dimethyl-4-oxo-chromen-8-yl)ethyl]amino]pyridine-2-carboxylate

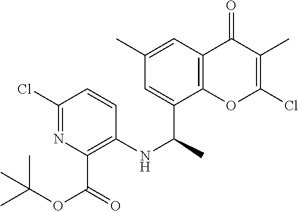 [SMILES: Cc1cc([C@@H](C)Nc2ccc(Cl)nc2C(=O)OC(C)(C)C)c2oc(Cl)c(C)c(=O)c2c1]

A solution of tert-butyl 6-chloro-3-[[(1R)-1-(2-ethylsulfinyl-3,6-dimethyl-4-oxo-chromen-8-yl)ethyl]amino]pyridine-2-carboxylate (0.95 g, 1.87 mmol) and benzyl(triethyl)ammonium chloride (0.21 g, 0.94 mmol) in DCM (30 mL) was treated with HCl (0.78 mL, 37% aqueous, 9.37 mmol). The reaction was stirred at rt for 1 h, treated with additional aqueous HCl (0.18 mL, 37% aqueous), and allowed to stir 30 min. The reaction was quenched with saturated aqueous NaHCO₃ and extracted with DCM. The organic layer was washed with saturated aqueous NaCl, collected, dried over MgSO₄, filtered, and concentrated under reduced pressure. The residue was purified by silica gel chromatography eluted with 0% to 50% EtOAc in heptane to give the title compound (0.42 g, 49%) as a white foam. ES/MS m/z 407 (MH-^{t}Bu).

The following compounds in Table 19 were made in a similar way as described for tert-butyl 6-chloro-3-[[(1R)-1-(2-chloro-3,6-dimethyl-4-oxo-chromen-8-yl)ethyl]amino]pyridine-2-carboxylate. Various methods were used to purify the compounds, which would be apparent to one skilled in the art.

TABLE 19

| | | | MS ES+ |
|---|---|---|---|
| Interm # | Chemical Name | Structure | m/z |
| 144C | Methyl 6-chloro-3-[[(1R)- 1-(2-chloro-3,6-dimethyl- 4-oxo-chromen-8-yl)- ethyl]amino]pyridine-2- carboxylate | 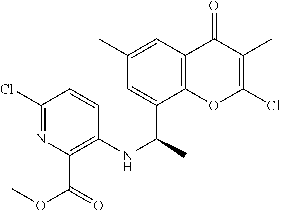 [SMILES: COC(=O)c1nc(Cl)ccc1N[C@H](C)c1cc(C)cc2c(=O)c(C)c(Cl)oc12] | 421 [M + H]⁺ |
| 145C | N-tert-Butyl-6-chloro-3- [[(1R)-1-(2-chloro-3,6- dimethyl-4-oxo-chromen- 8-yl)ethyl]amino]pyridine- 2-sulfonamide | 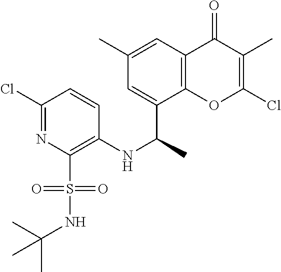 [SMILES: Cc1cc([C@@H](C)Nc2ccc(Cl)nc2S(=O)(=O)NC(C)(C)C)c2oc(Cl)c(C)c(=O)c2c1] | 498 [M + H]⁺ |

### Intermediate 146C: tert-Butyl 6-chloro-3-[[(1R)-1-[3,6-dimethyl-4-oxo-2-[3-(trifluoromethyl)-1-bicyclo[1.1.1]pentanyl]chromen-8-yl]ethyl]amino]pyridine-2-carboxylate

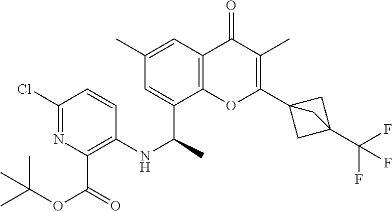 [SMILES: Cc1cc([C@@H](C)Nc2ccc(Cl)nc2C(=O)OC(C)(C)C)c2oc(C34CC(C(F)(F)F)(C3)C4)c(C)c(=O)c2c1]

A dry vial was charged with tert-butyl 6-chloro-3-[[(1R)-1-(2-chloro-3,6-dimethyl-4-oxo-chromen-8-yl)ethyl]amino]pyridine-2-carboxylate (208 mg, 0.45 mmol), palladium(II) acetate (10.1 mg, 0.05 mmol), and 2-dicyclohexylphosphino-2′,6′-bis(N,N-dimethylamino)biphenyl (CPhos, 39.2 mg, 0.09 mmol). The vial was evacuated and refilled with nitrogen three times. Added THF (2 mL) via syringe and cooled the reaction to 0° C. Added iodo-[3-(trifluoromethyl)-1-bicyclo[1.1.1]pentanyl]zinc (220 mg, ˜0.45 M in THF, 0.67 mmol) via syringe, removed the cooling bath, and stirred overnight at rt. The reaction was cooled to 0° C. and recharged with palladium(II) acetate (10.1 mg, 0.05 mmol), CPhos (39.2 mg, 0.09 mmol), and iodo-[3-(trifluoromethyl)-1-bicyclo[1.1.1]pentanyl]zinc (220 mg, 0.67 mmol). The cooling bath was removed and the reaction stirred at rt for 8 h. The reaction was diluted with EtOAc and washed with saturated aqueous NH₄Cl. The organic layer was removed and the aqueous layer re-extracted with EtOAc. The organic layers were combined, dried over Na₂SO₄, filtered, and concentrated under reduced pressure. The residue was purified by silica gel chromatography eluted with 0% to 50% EtOAc in heptane to give the title compound (183.2 mg, 73%) as a brown foam. ES/MS m/z 561 (M+H).

The following compounds in Table 20 were made in a similar way as described for tert-butyl 6-chloro-3-[[(1R)-1-[3,6-dimethyl-4-oxo-2-[3-(trifluoromethyl)-1-bicyclo[1.1.1]pentanyl]chromen-8-yl]ethyl]amino]pyridine-2-carboxylate. Various methods were used to purify the compounds, which would be apparent to one skilled in the art.

TABLE 20

| | | | MS ES+ |
|---|---|---|---|
| Interm # | Chemical Name | Structure | m/z |
| 147C | tert-Butyl 6-chloro-3-[[(1R)- 1-(2-cyclobutyl-3,6-dimeth- yl-4-oxo-chromen-8-yl)- ethyl]amino]pyridine-2- carboxylate | 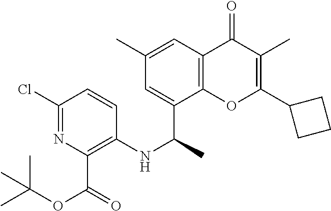 [SMILES: Cc1cc([C@@H](C)Nc2ccc(Cl)nc2C(=O)OC(C)(C)C)c2oc(C3CCC3)c(C)c(=O)c2c1] | 427 [MH − tBu]⁺ |
| 148C | tert-Butyl 6-chloro-3-[[(1R)- 1-[2-(4,4-difluorocyclohex- yl)-3,6-dimethyl-4-oxo- chromen-8-yl]ethyl]amino]- pyridine-2-carboxylate | 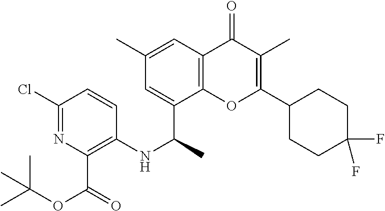 [SMILES: Cc1cc([C@@H](C)Nc2ccc(Cl)nc2C(=O)OC(C)(C)C)c2oc(C3CCC(F)(F)CC3)c(C)c(=O)c2c1] | 491 [MH − ^{t}Bu]⁺ |
| 149C | N-tert-Butyl-6-chloro-3- [[(1R)-1-[3,6-dimethyl-4- oxo-2-[3-(trifluoromethyl)- 1-bicyclo[1.1.1]pentanyl]- chromen-8-yl]ethyl]amino]- pyridine-2-sulfonamide | 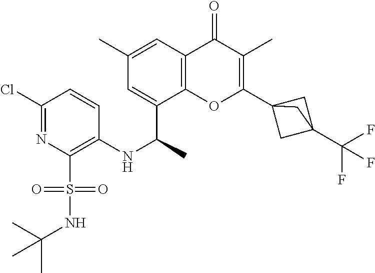 [SMILES: Cc1cc([C@@H](C)Nc2ccc(Cl)nc2S(=O)(=O)NC(C)(C)C)c2oc(C34CC(C(F)(F)F)(C3)C4)c(C)c(=O)c2c1] | 598 [M + H]⁺ |
| 150C | tert-Butyl 6-chloro-3-[(1R)- 1-(2-cyclopropyl-3,6- dimethyl-4-oxo-chromen-8- yl)ethyl]amino]pyridine-2- carboxylate | 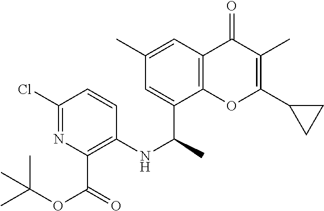 [SMILES: Cc1cc([C@@H](C)Nc2ccc(Cl)nc2C(=O)OC(C)(C)C)c2oc(C3CC3)c(C)c(=O)c2c1] | 413 [MH − ^{t}Bu]⁺ |
| 151C | N-tert-Butyl-6-chloro-3- [[(1R)-1-[2-(4,4-difluoro- cyclohexyl)-3,6-dimethyl- 4-oxo-chromen-8-yl]ethyl]- amino]pyridine-2-sulfon- amide | 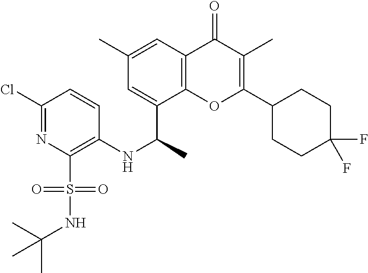 [SMILES: Cc1cc([C@@H](C)Nc2ccc(Cl)nc2S(=O)(=O)NC(C)(C)C)c2oc(C3CCC(F)(F)CC3)c(C)c(=O)c2c1] | 582 [M + H]⁺ |

### Intermediate 152C: 6-Chloro-3-[[(1R)-1-(2-ethylsulfanyl-3-iodo-6-methyl-4-oxo-chromen-8-yl)ethyl]amino]pyridine-2-carbonitrile

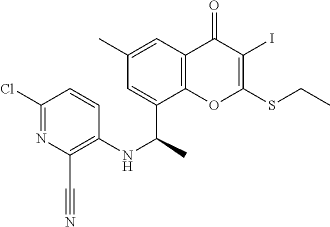 [SMILES: CCSc1oc2c([C@@H](C)Nc3ccc(Cl)nc3C#N)cc(C)cc2c(=O)c1I]

A solution of 6-chloro-3-[[(1R)-1-(2-ethylsulfanyl-6-methyl-4-oxo-chromen-8-yl)ethyl]amino]pyridine-2-carbonitrile (3.10 g, 7.75 mmol) in THF (50 mL) was purged with nitrogen gas and cooled to 0° C. The reaction was treated dropwise with 2,2,6,6-tetramethylpiperidinylzinc chloride lithium chloride complex solution (1M, 23.26 mL, 23.26 mmol). The reaction was stirred for 1 h while slowly warming to rt before iodine (5.90 g, 23.26 mmol) was added and the reaction stirred for another 30 min. The reaction was quenched with saturated aqueous NH₄Cl, concentrated under reduced pressure, and the residue resuspended in 100 mL of DCM. The suspension was filtered and the filtrate concentrated under reduced pressure. The residue was purified by silica gel chromatography eluted with 10% to 100% EtOAc in heptane to give the title compound (3.20 g, 75%) as a brown solid. ES/MS m/z 544 (M+H).

### Intermediate 153C: tert-Butyl 6-chloro-3-[[(1R)-1-(3-chloro-2-ethylsulfanyl-6-methyl-4-oxo-chromen-8-yl)ethyl]amino]pyridine-2-carboxylate

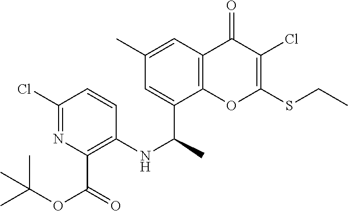 [SMILES: CCSc1oc2c([C@@H](C)Nc3ccc(Cl)nc3C(=O)OC(C)(C)C)cc(C)cc2c(=O)c1Cl]

Combined tert-butyl 6-chloro-3-[[(1R)-1-(2-ethylsulfanyl-6-methyl-4-oxo-chromen-8-yl)ethyl]amino]pyridine-2-carboxylate (600 mg, 1.26 mmol), N-chlorosuccinimide (202 mg, 1.52 mmol), and benzoyl peroxide (30.6 mg, 0.13 mmol) in ACN (10 mL) and stirred at 25° C. for 2 h. The reaction was concentrated under reduced pressure and purified by silica gel chromatography eluted with 10% to 100% EtOAc in heptane to give the title compound (580 mg, 90%) as a white solid. ES/MS m/z 453 (M+H).

### Intermediate 154C: 6-Chloro-3-[[(1R)-1-(2-ethylsulfanyl-3-isoxazol-4-yl-6-methyl-4-oxo-chromen-8-yl)ethyl]amino]pyridine-2-carbonitrile

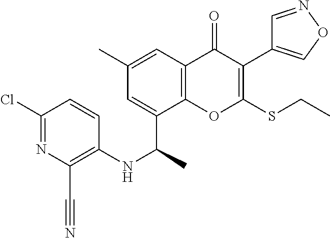 [SMILES: CCSc1oc2c([C@@H](C)Nc3ccc(Cl)nc3C#N)cc(C)cc2c(=O)c1-c1cnoc1]

Combined 6-chloro-3-[[(1R)-1-(2-ethylsulfanyl-3-iodo-6-methyl-4-oxo-chromen-8-yl)ethyl]amino]pyridine-2-carbonitrile (4.0 g, 7.61 mmol), 4-(4,4,5,5-tetramethyl-1,3,2-dioxaborolan-2-yl)isoxazole (2.97 g, 15.2 mmol), cesium carbonate (7.44 g, 22.8 mmol), and 1,1′-bis(di-tert-butylphosphino)ferrocene palladium(II) dichloride (0.50 g, 0.76 mmol) in 1,4-dioxane (90 mL) and water (10 mL). The reaction was stirred at 65° C. for 1.5 h. The reaction was cooled to rt, filtered through a pad of silica gel, and the filtrate concentrated under reduced pressure. The residue was purified by silica gel chromatography eluted with 10% to 80% EtOAc in hexane to give the title compound (3.1 g, 83%) as a white solid. ES/MS m/z 467 (M+H).

Intermediate-155C: 2-Ethylsulfanyl-8-[(1R)-1-[[2-(2-fluorophenyl)-3-pyridyl]amino]ethyl]-3,6-dimethyl-chromen-4-one

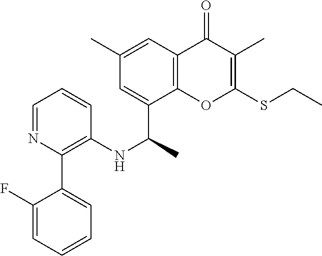 [SMILES: CCSc1oc2c([C@@H](C)Nc3cccnc3-c3ccccc3F)cc(C)cc2c(=O)c1C]

Combined 8-[(1R)-1-[(2-chloro-3-pyridyl)amino]ethyl]-2-ethylsulfanyl-3,6-dimethyl-chromen-4-one (1.37 g, 3.52 mmol), (2-fluorophenyl)boronic acid (0.49 g, 3.52 mmol), potassium carbonate (0.97 g, 7.05 mmol), and tetrakis(triphenylphosphine)palladium(0) (0.20 g, 0.17 mmol) in 1,4-dioxane (12 mL) and water (3 mL). Sparged the reaction with argon for 5 min and then stirred at 100° C. for 16 h. The reaction was purified by silica gel chromatography eluted with 0% to 60% EtOAc in heptane to give the title compound (1.10 g, 70%) as a yellow foam. ES/MS m/z 449 (M+H).

### Intermediate 118A: 8-[(1R)-1-Aminoethyl]-2-(2-fluorophenyl)-3,6-dimethyl-chromen-4-one

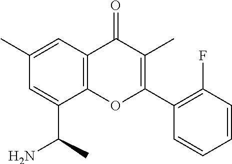 [SMILES: Cc1cc([C@@H](C)N)c2oc(-c3ccccc3F)c(C)c(=O)c2c1]

A solution of tert-butyl N-[(1R)-1-[2-(2-fluorophenyl)-3,6-dimethyl-4-oxo-chromen-8-yl]ethyl]carbamate (0.68 g, 1.65 mmol) in 10 mL of DCM was treated with TFA (5 mL) and the reaction stirred at 60° C. for 2 h. The reaction was concentrated and the residue purified by reversed phase C18 flash chromatography eluted with 0% to 100% ACN in 10 mM aqueous NH₄HCO₃ with 5% MeOH. Fractions containing product were pooled, washed with saturated aqueous sodium chloride, and extracted with IPA/CHCl₃ (1:3). The organics were collected, dried over MgSO₄, filtered, and concentrated to give the title compound (0.26 g, 51%) as a yellow foam. MS ES+ m/z 312 [M+H]⁺.

The following compounds in Table 21 were made in a similar way as described for 8-[(1R)-1-aminoethyl]-2-(2-fluorophenyl)-3,6-dimethyl-chromen-4-one. Various methods were used to purify the compounds, which would be apparent to one skilled in the art.

TABLE 21

| | | | MS ES+ |
|---|---|---|---|
| Interm # | Chemical Name | Structure | m/z |
| 119A | 8-[(1R)-1-Aminoethyl]-2- (2-fluoro-3-pyridyl)-3,6- dimethyl-chromen-4-one | 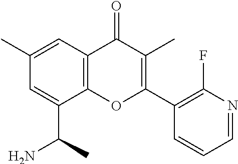 [SMILES: Cc1cc([C@@H](C)N)c2oc(-c3cccnc3F)c(C)c(=O)c2c1] | 313 [M + H]⁺ |
| 120A | 8-[(1R)-1-Aminoethyl]-2- (2,3-difluorophenyl)-3,6- dimethyl-chromen-4-one | 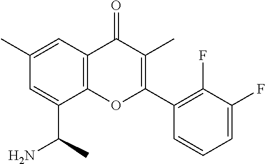 [SMILES: Cc1cc([C@@H](C)N)c2oc(-c3cccc(F)c3F)c(C)c(=O)c2c1] | 330 [M + H]⁺ |
| 121A | 8-[(1R)-1-Aminoethyl]-2- (2,4-difluorophenyl)-3,6- dimethyl-chromen-4-one | 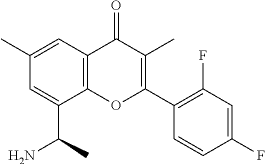 [SMILES: Cc1cc([C@@H](C)N)c2oc(-c3ccc(F)cc3F)c(C)c(=O)c2c1] | 330 [M + H]⁺ |
| 122A | 8-[(1R)-1-Aminoethyl]- 3,6-dimethyl-2-(2- pyridyl)chromen-4-one, 2,2,2-trifluoroacetic acid | 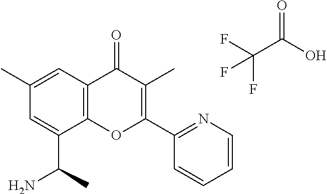 [SMILES: Cc1cc([C@@H](C)N)c2oc(-c3ccccn3)c(C)c(=O)c2c1 ; O=C(O)C(F)(F)F] | 295 [M + H]⁺ |
| 123A | 8-[(1R)-1-Aminoethyl]-2- (6-fluoro-2-pyridyl)-3,6- dimethyl-chromen-4-one | 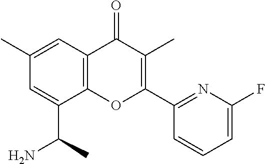 [SMILES: Cc1cc([C@@H](C)N)c2oc(-c3cccc(F)n3)c(C)c(=O)c2c1] | 313 [M + H]⁺ |
| 124A | 8-[(1R)-1-Aminoethyl]-2- (3-fluoro-2-pyridyl)-3,6- dimethyl-chromen-4-one | 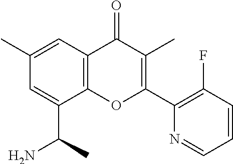 [SMILES: Cc1cc([C@@H](C)N)c2oc(-c3ncccc3F)c(C)c(=O)c2c1] | 313 [M + H]⁺ |
| 125A | 8-[(1R)-1-Aminoethyl]-2- (2,5-difluoro-3-pyridyl)- 3,6-dimethyl-chromen-4- one | 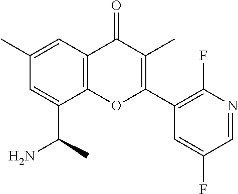 [SMILES: Cc1cc([C@@H](C)N)c2oc(-c3cc(F)cnc3F)c(C)c(=O)c2c1] | |
| 126A | 8-[(1R)-1-Aminoethyl]-2- (6-methoxy-2-pyridyl)- 3,6-dimethyl-chromen-4- one | 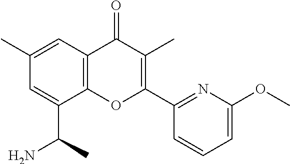 | 325 [M + H]⁺ |
| 127A | 8-[(1R)-1-Aminoethyl]-3- methyl-2-(2-pyridyl)-6- (trifluoromethyl)chromen- 4-one | 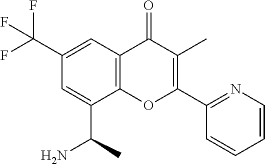 [SMILES: Cc1c(-c2ccccn2)oc2c([C@@H](C)N)cc(C(F)(F)F)cc2c1=O] | 349 [M + H]⁺ |
| 156C | 8-[(1R)-1-Aminoethyl]- 3,6-dimethyl-2-pyrazin-2- yl-chromen-4-one | 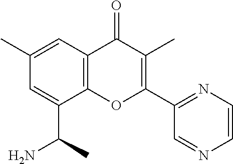 [SMILES: Cc1cc([C@@H](C)N)c2oc(-c3cnccn3)c(C)c(=O)c2c1] | 296 [M + H]⁺ |
| 157C | 5-[8-[(1R)-1-Aminoethyl]- 6-methyl-4-oxo-chromen- 2-yl]-1,3-dimethyl-pyridin- 2-one | 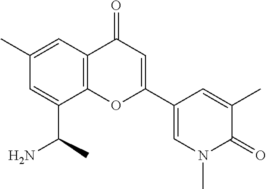 | 325 [M + H]⁺ |
| 158C | 5-[8-[(1R)-1-Aminoethyl]- 3,6-dimethyl-4-oxo-chro- men-2-yl]-1-isopropyl- pyridin-2-one | 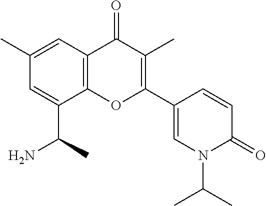 [SMILES: Cc1cc([C@@H](C)N)c2oc(-c3ccc(=O)n(C(C)C)c3)c(C)c(=O)c2c1] | 353 [M + H]⁺ |
| 159C | 5-[8-[(1R)-1-Aminoethyl]- 6-methyl-4-oxo-chromen- 2-yl]-1-methyl-pyridin-2- one | 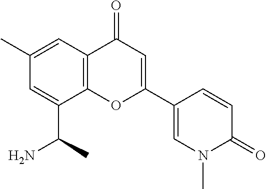 [SMILES: Cc1cc([C@@H](C)N)c2oc(-c3ccc(=O)n(C)c3)cc(=O)c2c1] | 311 [M + H]⁺ |
| 160C | 8-[(1R)-1-Aminoethyl]-6- methyl-3-oxazol-4-yl-2- phenyl-chromen-4-one | 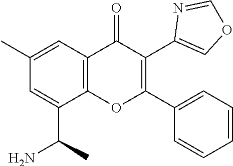 [SMILES: Cc1cc([C@@H](C)N)c2oc(-c3ccccc3)c(-c3cocn3)c(=O)c2c1] | 347 [M + H]⁺ |
| 161C | 8-[(1R)-1-Aminoethyl]-6- methyl-2-(1-methylpyr- azol-4-yl)-3-oxazol-4-yl- chromen-4-one | 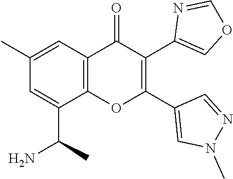 [SMILES: Cc1cc([C@@H](C)N)c2oc(-c3cnn(C)c3)c(-c3cocn3)c(=O)c2c1] | 351 [M + H]⁺ |
| 162C | 5-[8-[(1R)-1-Aminoethyl]- 3,6-dimethyl-4-oxo-chro- men-2-yl]-1-methyl-pyri- din-2-one | 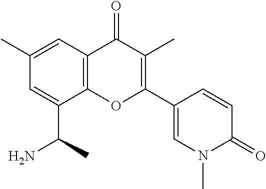 [SMILES: Cc1cc([C@@H](C)N)c2oc(-c3ccc(=O)n(C)c3)c(C)c(=O)c2c1] | 325 [M + H]⁺ |
| 163C | 8-[(1R)-1-Aminoethyl]-2- (3,6-difluoro-2-pyridyl)- 3,6-dimethyl-chromen-4- one | 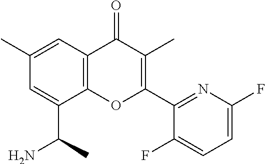 [SMILES: Cc1cc([C@@H](C)N)c2oc(-c3nc(F)ccc3F)c(C)c(=O)c2c1] | 331 [M + H]⁺ |
| 164C | 8-[(1R)-1-Aminoethyl]-2- (2,4-difluoro-3-pyridyl)- 3,6-dimethyl-chromen-4- one | 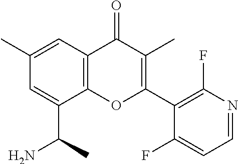 [SMILES: Cc1cc([C@@H](C)N)c2oc(-c3c(F)ccnc3F)c(C)c(=O)c2c1] | 331 [M + H]⁺ |
| 165C | 8-[(1R)-1-Aminoethyl]-2- (4-fluoro-3-pyridyl)-3,6- dimethyl-chromen-4-one | 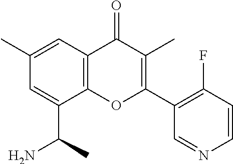 [SMILES: Cc1cc([C@@H](C)N)c2oc(-c3cnccc3F)c(C)c(=O)c2c1] | 313 [M + H]⁺ |
| 166C | 8-[(1R)-1-Aminoethyl]-2- (5-fluoro-2-pyridyl)-3,6- dimethyl-chromen-4-one | 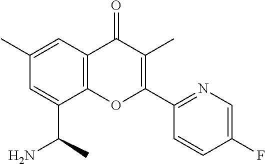 [SMILES: Cc1cc([C@@H](C)N)c2oc(-c3ccc(F)cn3)c(C)c(=O)c2c1] | 313 [M + H]⁺ |
| 167C | 8-[(1R)-1-Aminoethyl]- 3,6-dimethyl-2-pyrimidin- 2-yl-chromen-4-one | 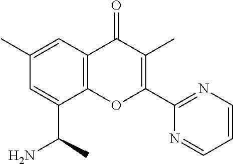 [SMILES: Cc1cc([C@@H](C)N)c2oc(-c3ncccn3)c(C)c(=O)c2c1] | 296 [M + H]⁺ |
| 168C | 8-[(1R)-1-Aminoethyl]-2- (6-methoxy-2-pyridyl)- 3,6-dimethyl-chromen-4- one | 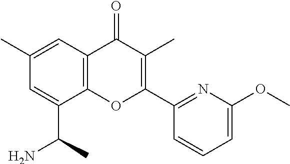 | 325 [M + H]⁺ |
| 169C | 8-[(1R)-1-Aminoethyl]-6- methyl-2-(2-pyridyl)- chromen-4-one | 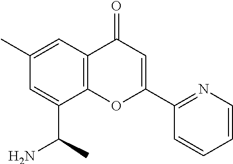 [SMILES: Cc1cc([C@@H](C)N)c2oc(-c3ccccn3)cc(=O)c2c1] | 281 [M + H]⁺ |
| 170C | 8-[(1R)-1-Aminoethyl]- 3,6-dimethyl-2-(2-methyl- pyrazolo[3,4-b]pyridin-5- yl)chromen-4-one | 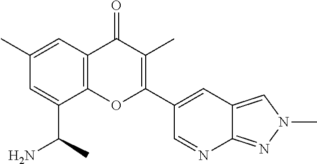 | 349 [M + H]⁺ |
| 171C | 8-[(1R)-1-Aminoethyl]-6- methyl-2-(3-pyridyl)- chromen-4-one | 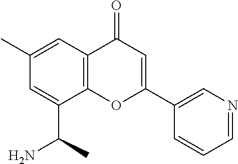 [SMILES: Cc1cc([C@@H](C)N)c2oc(-c3cccnc3)cc(=O)c2c1] | 281 [M + H]⁺ |
| 172C | 8-[(1R)-1-Aminoethyl]-2- (4-fluoro-2-pyridyl)-3,6- dimethyl-chromen-4-one | 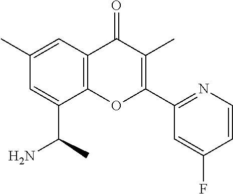 [SMILES: Cc1cc([C@@H](C)N)c2oc(-c3cc(F)ccn3)c(C)c(=O)c2c1] | 313 [M + H]⁺ |

### Intermediate 128A: 8-[(1R)-1-Aminoethyl]-6-methyl-2-(1-methylpyrazol-4-yl)chromen-4-one

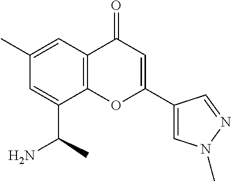 [SMILES: Cc1cc([C@@H](C)N)c2oc(-c3cnn(C)c3)cc(=O)c2c1]

A solution of tert-butyl N-[(1R)-1-[6-methyl-2-(1-methylpyrazol-4-yl)-4-oxo-chromen-8-yl]ethyl]carbamate (100 mg, 0.26 mmol) in 3 mL of DCM was treated with HCl (4 M in 1,4-dioxane, 190 mg, 5.22 mmol) and stirred at rt for 6 h. The reaction was concentrated and the residue purified by reverse phase chromatography eluted with 10% to 100% ACN (with 5% MeOH) in 10 mM aqueous NH₄HCO₃ to afford the title compound (54 mg, 69%) as a white solid. MS ES+ m/z 284 [M+H]⁺.

The following compounds in Table 22 were made in a similar way as described for 8-[(1R)-1-aminoethyl]-6-methyl-2-(1-methylpyrazol-4-yl)chromen-4-one. Various methods were used to purify the compounds, which would be apparent to one skilled in the art.

TABLE 22

| | | | MS ES+ |
|---|---|---|---|
| Interm # | Chemical Name | Structure | m/z |
| 173C | 8-[(1R)-1-Aminoethyl]-6- methyl-2-phenyl-chromen- 4-one | 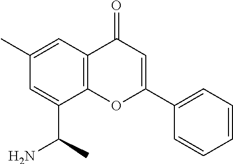 [SMILES: Cc1cc([C@@H](C)N)c2oc(-c3ccccc3)cc(=O)c2c1] | 280 [M + H]⁺ |
| 174C | 4-[8-[(1R)-1-Aminoethyl]- 3,6-dimethyl-4-oxo-chro- men-2-yl]-1-methyl- pyridin-2-one | 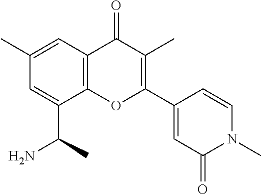 | 649 [2M + H]⁺ |
| 175C | 8-[(1R)-1-Aminoethyl]- 3,6-dimethyl-2-(1-methyl- pyrazolo[4,3-b]pyridin-5- yl)chromen-4-one | 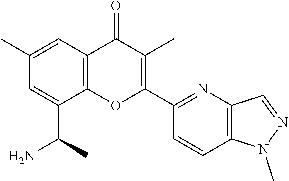 [SMILES: Cc1cc([C@@H](C)N)c2oc(-c3ccc4c(cnn4C)n3)c(C)c(=O)c2c1] | 349 [M + H]⁺ |
| 176C | 8-[(1R)-1-Aminoethyl]- 6-methyl-2-(2-methyl- indazol-5-yl)chromen-4- one | 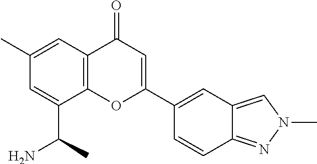 | 334 [M + H]⁺ |
| 177C | 8-[(1R)-1-Aminoethyl]- 3,6-dimethyl-2-(1-methyl- pyrazol-4-yl)chromen-4- one | 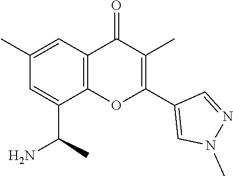 [SMILES: Cc1cc([C@@H](C)N)c2oc(-c3cnn(C)c3)c(C)c(=O)c2c1] | 298 [M + H]⁺ |
| 178C | 8-[(1R)-1-Aminoethyl]- 3,6-dimethyl-2-(2-methyl- indazol-5-yl)chromen-4- one | 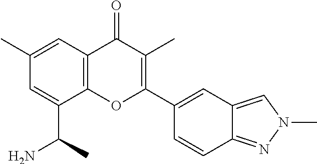 | 348 [M + H]⁺ |
| 179C | 8-[(1R)-1-Aminoethyl]- 3,6-dimethyl-2-(2-methyl- pyrazolo[4,3-b]pyridin-5- yl)chromen-4-one | 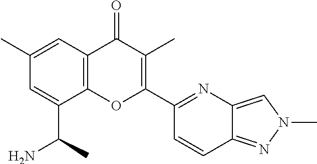 | 349 [M + H]⁺ |

### Intermediate 14A: 2-[[(1R)-1-(3,6-Dimethyl-4-oxo-2-phenyl-chromen-8-yl)ethyl]amino]benzoic acid

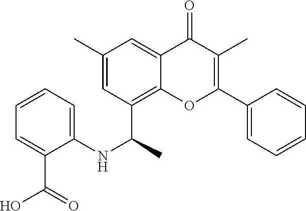 [SMILES: Cc1cc([C@@H](C)Nc2ccccc2C(=O)O)c2oc(-c3ccccc3)c(C)c(=O)c2c1]

A mixture of 8-[(1R)-1-aminoethyl]-3,6-dimethyl-2-phenyl-chromen-4-one (30 g, 102.3 mmol), 2-iodobenzoic acid (25.36 g, 102.26 mmol), copper (13.00 g, 204.5 mmol), and potassium carbonate (21.20 g, 153.4 mmol) was suspended in DMF (300 mL) and stirred at 100° C. for 3 h. A mixture of 8-[(1R)-1-aminoethyl]-3,6-dimethyl-2-phenyl-chromen-4-one (30 g, 102.3 mmol), 2-iodobenzoic acid (25.36 g, 102.26 mmol), copper (13.00 g, 204.5 mmol), and potassium carbonate (21.20 g, 153.4 mmol) was suspended in DMF (300 mL) and stirred at 100° C. for 3 h. The two reactions were combined, the pH adjusted to ˜3 with 2M aqueous HCl, diluted with DCM (1 L) and water (500 mL), filtered, and the layers separated. The organic layer was washed with saturated aqueous sodium chloride (3×1 L), dried over Na₂SO₄, and concentrated. The residue was purified by silica gel chromatography (5-50% EtOAc in petroleum ether). The resulting solid was triturated with EtOAc (300 mL) and collected by filtration. The resulting solid was suspended in boiling I (500 mL) and collected by filtration to give the product (33 g; 39%) as a white solid. MS ES+ m/z 414 [M+H]⁺.

### Intermediate 129A: N-tert-Butyl-6-chloro-3-[[(1R)-1-[3,6-dimethyl-4-oxo-2-(3-pyridyl)chromen-8-yl]ethyl]amino]pyridine-2-sulfonamide

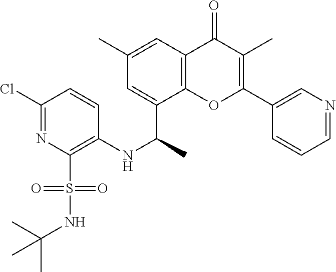 [SMILES: Cc1cc([C@@H](C)Nc2ccc(Cl)nc2S(=O)(=O)NC(C)(C)C)c2oc(-c3cccnc3)c(C)c(=O)c2c1]

A mixture of 8-[(1R)-1-aminoethyl]-3,6-dimethyl-2-(3-pyridyl)chromen-4-one (0.60 g, 2.04 mmol) and 3-bromo-N-tert-butyl-6-chloro-pyridine-2-sulfonamide (0.80 g, 2.45 mmol) in 6 mL of DMSO was treated with 4,7-dimethoxy-1,10-phenanthroline (97 mg, 0.41 mmol), cesium carbonate (1.33 g, 4.08 mmol), and copper(I) iodide (38 mg, 0.20 mmol) in portions at rt under nitrogen. The reaction was heated to 150° C. for 30 min in a microwave. The reaction was allowed to cool to rt, filtered, and the solids washed with EtOAc (3×10 mL). The filtrate was diluted with water (50 mL) and extracted with EtOAc (3×30 mL). The combined organics were washed with saturated aqueous sodium chloride (3×30 mL), collected, dried over Na₂SO₄, filtered, and concentrated. The residue was purified by reversed phase chromatography eluted with 40% to 70% ACN in water (with 0.1% formic acid) to give the title compound (0.18 g, 160%) as a yellow solid. MS ES+ m/z 541 [M+H]⁺.

The following compounds in Table 23 were made in a similar way as described for N-tert-butyl-6-chloro-3-[[(1R)-1-[3,6-dimethyl-4-oxo-2-(3-pyridyl)chromen-8-yl]ethyl]amino]pyridine-2-sulfonamide. Various methods were used to purify the compounds, which would be apparent to one skilled in the art.

TABLE 23

| | | | MS ES+ |
|---|---|---|---|
| Interm # | Chemical Name | Structure | m/z |
| 180C | N-tert-Butyl-6-chloro-3- [[(1R)-1-[6-methyl-2- (2-methylindazol-5-yl)- 4-oxo-chromen-8-yl]- ethyl]amino]pyridine- 2-sulfonamide | 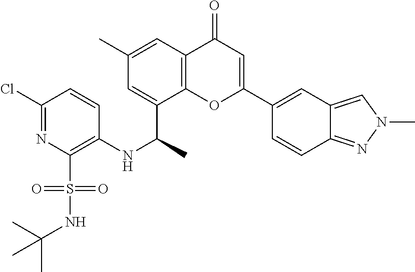 [SMILES: Cc1cc([C@@H](C)Nc2ccc(Cl)nc2S(=O)(=O)NC(C)(C)C)c2oc(-c3ccc4nn(C)cc4c3)cc(=O)c2c1] | 580 [M + H]⁺ |
| 181C | N-tert-Butyl-6-chloro- 3-[[(1R)-1-[3,6-dimeth- yl-2-(1-methylpyrazol- 4-yl)-4-oxo-chromen-8- yl]ethyl]amino]pyridine- 2-sulfonamide | 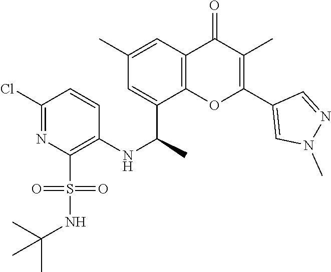 [SMILES: Cc1cc([C@@H](C)Nc2ccc(Cl)nc2S(=O)(=O)NC(C)(C)C)c2oc(-c3cnn(C)c3)c(C)c(=O)c2c1] | 544 [M + H]⁺ |
| 182C | N-tert-Butyl-6-chloro-3- [[(1R)-1-[3,6-dimethyl- 2-(2-methylindazol-5- yl)-4-oxo-chromen-8-yl]- ethyl]amino]pyridine-2- sulfonamide | 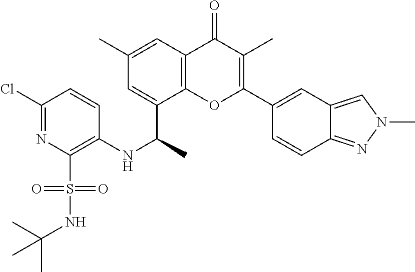 [SMILES: Cc1cc([C@@H](C)Nc2ccc(Cl)nc2S(=O)(=O)NC(C)(C)C)c2oc(-c3ccc4nn(C)cc4c3)c(C)c(=O)c2c1] | 594 [M + H]⁺ |

### Intermediate 183C: N-tert-Butyl-6-chloro-3-[[(1R)-1-[2-(1,5-dimethyl-6-oxo-3-pyridyl)-6-methyl-4-oxo-chromen-8-yl]ethyl]amino]pyridine-2-sulfonamide

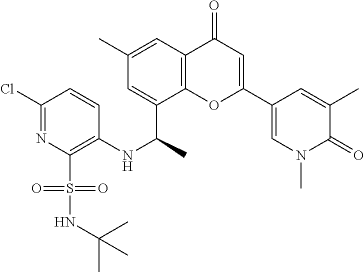 [SMILES: Cc1cc([C@@H](C)Nc2ccc(Cl)nc2S(=O)(=O)NC(C)(C)C)c2oc(-c3cc(C)c(=O)n(C)c3)cc(=O)c2c1]

A solution of 5-[8-[(1R)-1-aminoethyl]-6-methyl-4-oxo-chromen-2-yl]-1,3-dimethyl-pyridin-2-one (200 mg, 0.62 mmol) and 3-bromo-N-tert-butyl-6-chloro-pyridine-2-sulfonamide (404 mg, 1.23 mmol) in DMF (6 mL) was treated with cesium carbonate (402 mg, 1.23 mmol) and copper(I) iodide (23.5 mg, 0.12 mmol) in portions at rt under a nitrogen atmosphere. The reaction was stirred at 120° C. overnight. The mixture was concentrated under reduced pressure and the residue purified by reversed phase chromatography on C18 eluted with 20% to 50% ACN in water (with 0.1% NH₄OH) to give the title compound (88 mg, 25% as a white solid. ES/MS m/z 571 (M+H).

The following compounds in Table 24 were made in a similar way as described for N-tert-butyl-6-chloro-3-[[(1R)-1-[2-(1,5-dimethyl-6-oxo-3-pyridyl)-6-methyl-4-oxo-chromen-8-yl]ethyl]amino]pyridine-2-sulfonamide. Various methods were used to purify the compounds, which would be apparent to one skilled in the art.

TABLE 24

| | | | MS ES+ |
|---|---|---|---|
| Interm # | Chemical Name | Structure | m/z |
| 184C | N-tert-Butyl-6-chloro-3- [[(1R)-1-(6-methyl-4- oxo-2-phenyl-chromen-8- yl)ethyl]amino]pyridine- 2-sulfonamide | 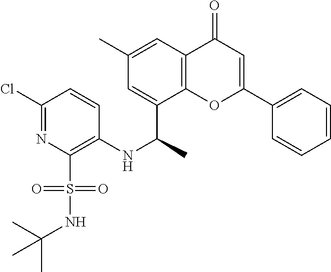 [SMILES: Cc1cc([C@@H](C)Nc2ccc(Cl)nc2S(=O)(=O)NC(C)(C)C)c2oc(-c3ccccc3)cc(=O)c2c1] | 526 [M + H]⁺ |
| 185C | N-tert-Butyl-6-chloro-3- [[(1R)-1-[2-(1-isopropyl- 6-oxo-3-pyridyl)-3,6- dimethyl-4-oxo-chro- men-8-yl]ethyl]amino]- pyridine-2-sulfonamide | 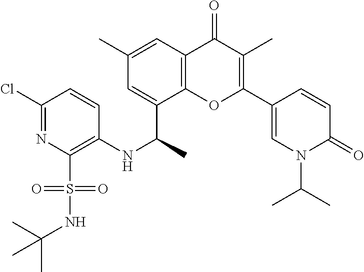 [SMILES: Cc1cc([C@@H](C)Nc2ccc(Cl)nc2S(=O)(=O)NC(C)(C)C)c2oc(-c3ccc(=O)n(C(C)C)c3)c(C)c(=O)c2c1] | 599 [M + H]⁺ |
| 186C | N-tert-Butyl-6-chloro-3- [[(1R)-1-[6-methyl-2-(1- methylpyrazol-4-yl)-4- oxo-chromen-8-yl]- ethyl]amino]pyridine-2- sulfonamide | 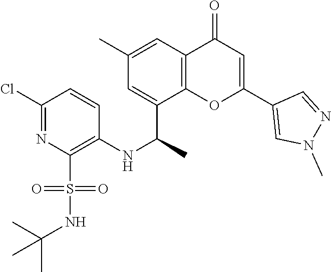 [SMILES: Cc1cc([C@@H](C)Nc2ccc(Cl)nc2S(=O)(=O)NC(C)(C)C)c2oc(-c3cnn(C)c3)cc(=O)c2c1] | 530 [M + H]⁺ |
| 187C | N-tert-Butyl-3-[[(1R)- 1-[3,6-dimethyl-2-(1- methylpyrazol-4-yl)- 4-oxo-chromen-8-yl]- ethyl]amino]pyridine- 2-sulfonamide | 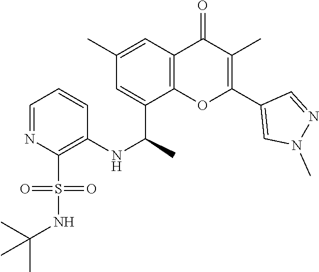 [SMILES: Cc1cc([C@@H](C)Nc2cccnc2S(=O)(=O)NC(C)(C)C)c2oc(-c3cnn(C)c3)c(C)c(=O)c2c1] | 510 [M + H]⁺ |
| 188C | N-tert-Butyl-6-chloro-3- [[(1R)-1-[3,6-dimethyl- 4-oxo-2-(2-pyridyl)- chromen-8-yl]ethyl]- amino]pyridine-2- sulfonamide | 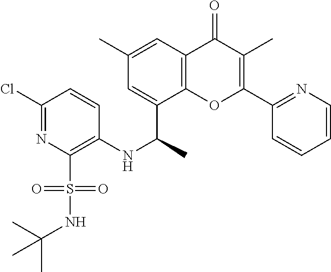 [SMILES: Cc1cc([C@@H](C)Nc2ccc(Cl)nc2S(=O)(=O)NC(C)(C)C)c2oc(-c3ccccn3)c(C)c(=O)c2c1] | 541 [M + H]⁺ |

### Intermediate 189C: 6-Chloro-3-[[(1R)-1-[3,6-dimethyl-2-(2-methylpyrazolo[4,3-b]pyridin-5-yl)-4-oxo-chromen-8-yl]ethyl]amino]pyridine-2-carbonitrile

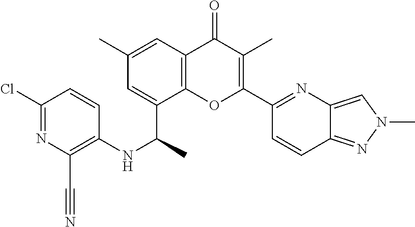 [SMILES: Cc1cc([C@@H](C)Nc2ccc(Cl)nc2C#N)c2oc(-c3ccc4nn(C)cc4n3)c(C)c(=O)c2c1]

A solution of 8-[(1R)-1-aminoethyl]-3,6-dimethyl-2-(2-methylpyrazolo[4,3-b]pyridin-5-yl)chromen-4-one (0.57 g, 1.64 mmol) in DMF (10 mL) was treated with 6-chloro-3-fluoro-pyridine-2-carbonitrile (0.77 g, 4.91 mmol), potassium carbonate (2.26 g, 16.36 mmol), and DIPEA (0.63 g, 4.91 mmol) at rt. The reaction was stirred at 100° C. for 2 h. After cooling to rt, the reaction was diluted with water (100 mL) and extracted with EtOAc (3×100 mL). The combined organic layers were washed with saturated aqueous NaCl, dried over Na₂SO₄, filtered, and concentrated under reduced pressure. The residue was purified by silica gel chromatography eluted with 99% EtOAc in petroleum ether to give the title compound (0.25 g, 32%) as a brown solid. ES/MS m/z 485 (M+H).

The following compound in Table 25 was made in a similar way as described for 6-chloro-3-[[(1R)-1-[3,6-dimethyl-2-(2-methylpyrazolo[4,3-b]pyridin-5-yl)-4-oxo-chromen-8-yl]ethyl]amino]pyridine-2-carbonitrile. Various methods were used to purify the compound, which would be apparent to one skilled in the art.

TABLE 25

| | | | MS ES+ |
|---|---|---|---|
| Interm # | Chemical Name | Structure | m/z |
| 190C¹ | Methyl 6-chloro-3- [[(1R)-1-[3,6-dimethyl- 2-(2-methylpyrazolo- [4,3-b]pyridin-5-yl)-4- oxo-chromen-8-yl]- ethyl]amino]pyridine- 2-carboxylate | 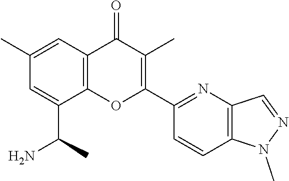 | 518 [M + H]⁺ |

¹No DIPEA used in the reaction.

### Intermediate 130A: tert-Butyl 2-[[(1R)-1-(6-methyl-4-oxo-2-phenyl-chromen-8-yl)ethyl]amino]benzoate

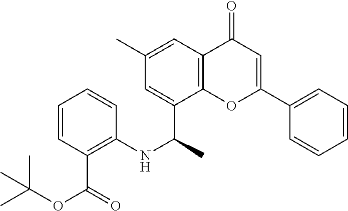 [SMILES: Cc1cc([C@@H](C)Nc2ccccc2C(=O)OC(C)(C)C)c2oc(-c3ccccc3)cc(=O)c2c1]

Combined tert-butyl 2-[[(1R)-1-(2-ethylsulfanyl-6-methyl-4-oxo-chromen-8-yl)ethyl]amino]benzoate (1.00 g, 2.27 mmol; Intermediate 106, tert-butyl 2-[1-(2-ethylsulfanyl-6-methyl-4-oxo-chromen-8-yl)ethylamino]benzoate, Isomer 2), phenylboronic acid (0.56 g, 4.55 mmol), tris(dibenzylideneacetone)dipalladium(0) (0.21 g, 0.23 mmol), copper(I) thiophene-2-carboxylate (0.87 g, 4.55 mmol), zinc(II) acetate (0.84 g, 4.55 mmol), and tri(2-furyl)phosphine (0.26 g, 1.14 mmol) in 1,4-dioxane (20 mL) and heated at 85° C. for 12 h. The crude product mixture was purified by silica gel chromatography eluted with 10% to 60% EtOAc in heptane, then reversed phase chromatography eluted with 0% to 100% ACN in water (with 0.1% TFA) to afford the product (0.53 g, 48%). MS ES+ m/z 456 [M+H]⁺.

The following compound in Table 26 was made in a similar way as described for tert-butyl 2-[[(1R)-1-(6-methyl-4-oxo-2-phenyl-chromen-8-yl)ethyl]amino]benzoate. Various methods were used to purify the compound, which would be apparent to one skilled in the art.

TABLE 26

| | | | MS ES+ |
|---|---|---|---|
| Interm # | Chemical Name | Structure | m/z |
| 191C | tert-Butyl 2-[8-[1-(2-tert- butoxycarbonylanilino)- ethyl]-6-fluoro-4-oxo- chromen-2-yl]indole-1- carboxylate, Isomer 1 | 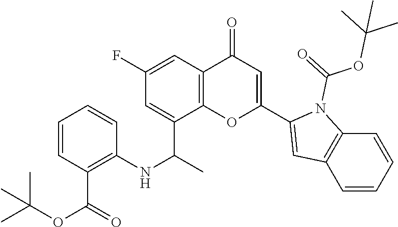 [SMILES: CC(Nc1ccccc1C(=O)OC(C)(C)C)c1cc(F)cc2c(=O)cc(-c3cc4ccccc4n3C(=O)OC(C)(C)C)oc12] | 599 [M + H]⁺ |

### Intermediate 192C: 6-Chloro-3-[[(1R)-1-[3,6-dimethyl-4-oxo-2-(1H-pyrazol-4-yl)chromen-8-yl]ethyl]amino]pyridine-2-carbonitrile

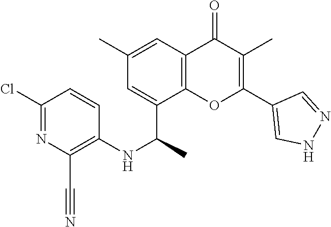 [SMILES: Cc1cc([C@@H](C)Nc2ccc(Cl)nc2C#N)c2oc(-c3cn[nH]c3)c(C)c(=O)c2c1]

A vial containing 6-chloro-3-[[(1R)-1-(2-ethylsulfanyl-3,6-dimethyl-4-oxo-chromen-8-yl)ethyl]amino]pyridine-2-carbonitrile (0.31 g, 0.74 mmol), 1H-pyrazol-4-ylboronic acid, (0.25 g, 2.23 mmol), tetrakis(triphenylphosphine)palladium (0) (0.21 g, 0.19 mmol), copper(I) 3-methylsalicylate (0.24 g, 1.11 mmol) was suspended in EtOH (3 mL) and THF (3 mL). Nitrogen was bubbled through the reaction for 2 min, the vial was sealed, and the reaction stirred at 60° C. for 4 h. The reaction was recharged with the same amounts of 1H-pyrazol-4-ylboronic acid, tetrakis(triphenylphosphine)palladium (0), and copper(I) 3-methylsalicylate. Nitrogen was bubbled through the reaction for 5 min, the vial sealed, and the reaction stirred at 60° C. for 16 h. The reaction was cooled, filtered through diatomaceous earth, and solids washed with DCM. The filtrate was concentrated under reduced pressure and the residue purified by reversed phase chromatography on C18 eluted with 16% to 44% ACN in water (with 0.1% formic acid) to give the title compound (60 mg, 19%). ES/MS m/z 420 (M+H).

The following compounds in Table 27 were made in a similar way as described for 6-chloro-3-[[(1R)-1-[3,6-dimethyl-4-oxo-2-(1H-pyrazol-4-yl)chromen-8-yl]ethyl]amino]pyridine-2-carbonitrile. Various methods were used to purify the compounds, which would be apparent to one skilled in the art.

TABLE 27

| | | | MS ES+ |
|---|---|---|---|
| Interm # | Chemical Name | Structure | m/z |
| 193C | 6-Chloro-3-[[(1R)-1- [3,6-dimethyl-4-oxo-2- [1-(trifluoromethyl)- pyrazol-4-yl]chromen- 8-yl]ethyl]amino]pyri- dine-2-carbonitrile | 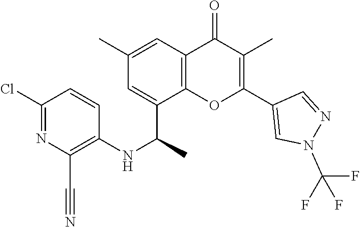 [SMILES: Cc1cc([C@@H](C)Nc2ccc(Cl)nc2C#N)c2oc(-c3cnn(C(F)(F)F)c3)c(C)c(=O)c2c1] | 486 [M − H]⁻ |
| 194C | 6-Chloro-3-[(1R)-1- [2-(6-fluoro-2-pyrid- yl)-3,6-dimethyl-4- oxo-chromen-8-yl]- ethyl]amino]pyridine- 2-carboxamide | 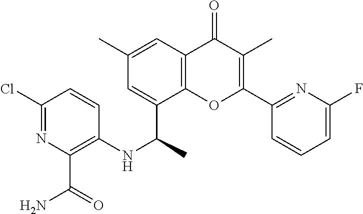 [SMILES: Cc1cc([C@@H](C)Nc2ccc(Cl)nc2C(N)=O)c2oc(-c3cccc(F)n3)c(C)c(=O)c2c1] | 467 [M + H]⁺ |
| 195C | tert-Butyl 3-[[(1R)-1- [2-(1,3-benzodioxol- 5-yl)-3,6-dimethyl-4- oxo-chromen-8-yl]- ethyl]amino]-6- chloro-pyridine-2- carboxylate | 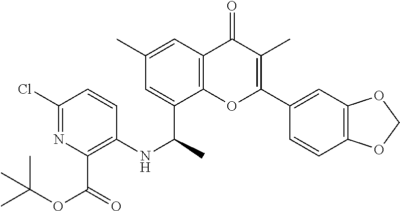 [SMILES: Cc1cc([C@@H](C)Nc2ccc(Cl)nc2C(=O)OC(C)(C)C)c2oc(-c3ccc4c(c3)OCO4)c(C)c(=O)c2c1] | 493 [MH − 'Bu]⁺ |
| 196C | 6-Chloro-3-[(1R)-1- [2-[1-(2-hydroxy-2- methyl-propyl)pyrazol- 4-yl]-3,6-dimethyl-4- oxo-chromen-8-yl]- ethyl]amino]pyridine- 2-carbonitrile | 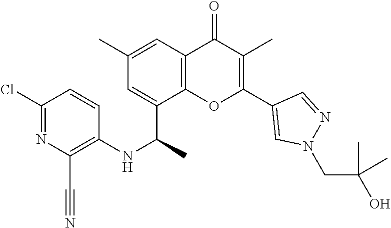 [SMILES: Cc1cc([C@@H](C)Nc2ccc(Cl)nc2C#N)c2oc(-c3cnn(CC(C)(C)O)c3)c(C)c(=O)c2c1] | 492 [M + H]⁺ |
| 197C | tert-Butyl 3-[[(1R)-1- [3,6-dimethyl-2-(1- methylpyrazol-4-yl)- 4-oxo-chromen-8-yl]- ethyl]amino]-6-fluoro- pyridine-2-carboxylate | 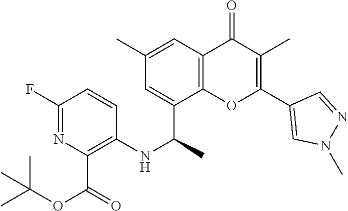 [SMILES: Cc1cc([C@@H](C)Nc2ccc(F)nc2C(=O)OC(C)(C)C)c2oc(-c3cnn(C)c3)c(C)c(=O)c2c1] | 493 [M + H]⁺ |
| 198C | 6-Chloro-3-[(1R)-1- [3,6-dimethyl-4-oxo- 2-(1-tetrahydropyran- 4-ylpyrazol-4-yl)- chromen-8-yl]ethyl]- amino]pyridine-2- carbonitrile | 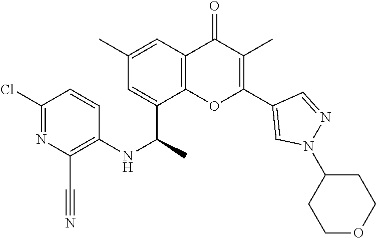 [SMILES: Cc1cc([C@@H](C)Nc2ccc(Cl)nc2C#N)c2oc(-c3cnn(C4CCOCC4)c3)c(C)c(=O)c2c1] | 504 [M + H]⁺ |
| 199C¹ | tert-Butyl N-[(1R)-1- [2-(2,4-difluoro-3- pyridyl)-3,6-dimethyl- 4-oxo-chromen-8-yl]- ethyl]carbamate | 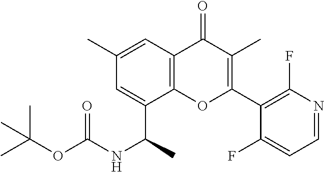 [SMILES: Cc1cc([C@@H](C)NC(=O)OC(C)(C)C)c2oc(-c3c(F)ccnc3F)c(C)c(=O)c2c1] | 431 [M + H]⁺ |
| 200C | tert-Butyl 6-chloro-3- [[(1R)-1-[3-chloro-6- methyl-2-(1-methyl- pyrazol-4-yl)-4-oxo- chromen-8-yl]ethyl]- amino]pyridine-2- carboxylate | 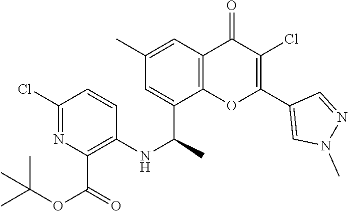 [SMILES: Cc1cc([C@@H](C)Nc2ccc(Cl)nc2C(=O)OC(C)(C)C)c2oc(-c3cnn(C)c3)c(Cl)c(=O)c2c1] | 473 [MH − ^{t}Bu]⁺ |
| 201C | tert-Butyl 6-chloro-3- [1-[6-fluoro-3-methyl- 2-(1-methylpyrazol-4- yl)-4-oxo-chromen-8- yl]ethylamino]pyri- dine-2-carboxylate, Isomer 1 | 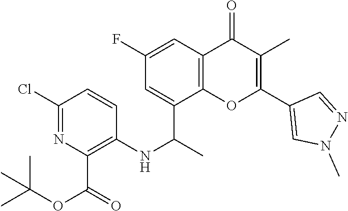 [SMILES: Cc1c(-c2cnn(C)c2)oc2c(C(C)Nc3ccc(Cl)nc3C(=O)OC(C)(C)C)cc(F)cc2c1=O] | 457 [MH − ^{t}Bu]⁺ |

¹Stannane used as coupling partner.

### Intermediate 15A: tert-Butyl 3-[[(1R)-1-(3,6-dimethyl-4-oxo-2-phenyl-chromen-8-yl)ethyl]amino]pyridine-2-carboxylate

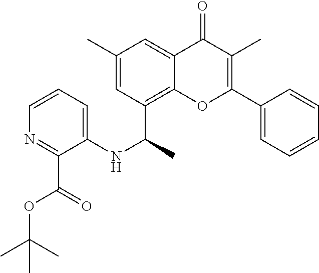 [SMILES: Cc1cc([C@@H](C)Nc2cccnc2C(=O)OC(C)(C)C)c2oc(-c3ccccc3)c(C)c(=O)c2c1]

Combined tris(dibenzylideneacetone)dipalladium(0) (1.17 g, 1.28 mmol), 4,5-bis(diphenylphosphino)-9,9-dimethylxanthene (1.97 g, 3.41 mmol), (8-[(1R)-1-aminoethyl]-3,6-dimethyl-2-phenyl-chromen-4-one (5.00 g, 17.0 mmol), tert-butyl 3-bromopicolinate (5.28 g, 20.5 mmol), and Cs₂CO₃ (13.9 g, 42.6 mmol) in toluene (80 mL). The reaction was heated to 115° C. for 16 h. The mixture was filtered over diatomaceous earth and the solids were rinsed with EtOAc and DCM. The filtrate was concentrated and the residue was purified by silica gel chromatography eluted with a gradient of 0 to 80% EtOAc in heptane to give the title compound (7.1 g, 89%). MS ES+ m/z 471 [M+H]⁺.

The following compounds in Table 28 were made in a similar way as described for tert-butyl 3-[[(1R)-1-(3,6-dimethyl-4-oxo-2-phenyl-chromen-8-yl)ethyl]amino]pyridine-2-carboxylate. Various methods were used to purify the compounds, which would be apparent to one skilled in the art.

TABLE 28

| | | | MS ES+ |
|---|---|---|---|
| Interm # | Chemical Name | Structure | m/z |
| 16A | 3-[[(1R)-1-(3,6-Dimethyl-4- oxo-2-phenyl-chromen-8- yl)ethyl]amino]pyridine-2- carbonitrile | 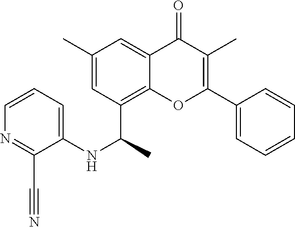 [SMILES: Cc1cc([C@@H](C)Nc2cccnc2C#N)c2oc(-c3ccccc3)c(C)c(=O)c2c1] | 396 [M + H]⁺ |
| 17A | 6-Chloro-3-[[(1R)-1-(3,6- dimethyl-4-oxo-2-phenyl- chromen-8- yl)ethyl]amino]pyridine-2- carbonitrile | 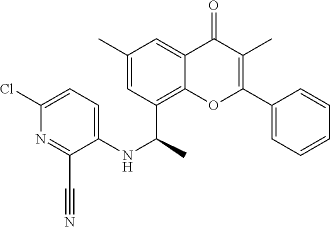 [SMILES: Cc1cc([C@@H](C)Nc2ccc(Cl)nc2C#N)c2oc(-c3ccccc3)c(C)c(=O)c2c1] | 430 [M + H]⁺ |
| 18A | 8-[(1R)-1-(2- Bromoanilino)ethyl]-3,6- dimethyl-2-phenyl-chromen- 4-one | 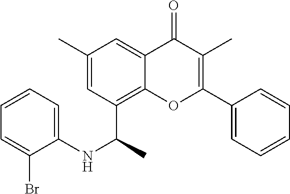 [SMILES: Cc1cc([C@@H](C)Nc2ccccc2Br)c2oc(-c3ccccc3)c(C)c(=O)c2c1] | 450 [M + H]⁺ |
| 19A | 3-[[(1R)-1-(3,6-Dimethyl-4- oxo-2-phenyl-chromen-8- yl)ethyl]amino]-6-methoxy- pyridine-6-carbonitrile | 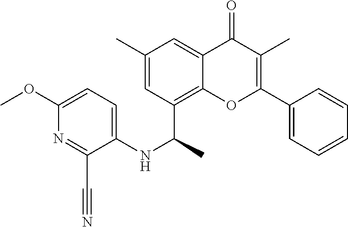 [SMILES: COc1ccc(N[C@H](C)c2cc(C)cc3c(=O)c(C)c(-c4ccccc4)oc23)c(C#N)n1] | 426 [M + H]⁺ |
| 20A | 3-[[(1R)-1-(3,6-Dimethyl-4- oxo-2-phenyl-chromen-8- yl)ethyl]amino]-6-methyl- pyridine-2-carbonitrile | 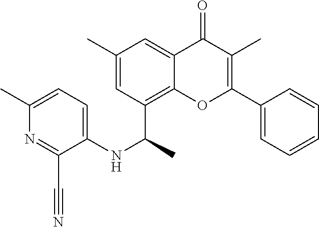 [SMILES: Cc1cc([C@@H](C)Nc2ccc(C)nc2C#N)c2oc(-c3ccccc3)c(C)c(=O)c2c1] | 410 [M + H]⁺ |
| 21A | 8-[(1R)-1-[(2-Chloro-3- pyridyl)amino]ethyl]-3,6- dimethyl-2-phenyl-chromen- 4-one | 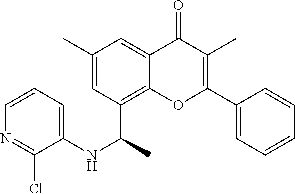 [SMILES: Cc1cc([C@@H](C)Nc2cccnc2Cl)c2oc(-c3ccccc3)c(C)c(=O)c2c1] | 405 [M + H]⁺ |
| 22A | 2-[[(1R)-1-(3,6-Dimethyl-4- oxo-2-phenyl-chromen-8- yl)ethyl]amino]pyridine-3- carbonitrile | 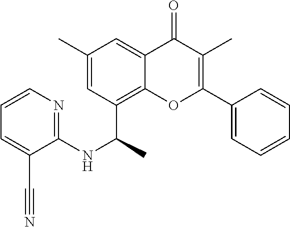 [SMILES: Cc1cc([C@@H](C)Nc2ncccc2C#N)c2oc(-c3ccccc3)c(C)c(=O)c2c1] | 396 [M + H]⁺ |
| 23A | 2-[[(1R)-1-(2-Ethylsulfanyl- 3,6-dimethyl-4-oxo- chromen-8- yl)ethyl]amino]benzonitrile | 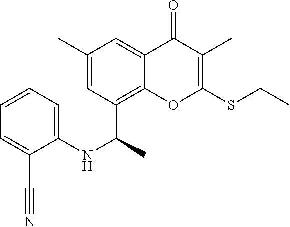 [SMILES: CCSc1oc2c([C@@H](C)Nc3ccccc3C#N)cc(C)cc2c(=O)c1C] | 379 [M + H]⁺ |
| 24A | 2-Ethylsulfanyl-3,6- dimethyl-8-[(1R)-1-[[2- (trifluoromethyl)-3- pyridyl]amino]ethyl] chromen-4-one | 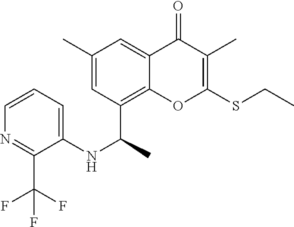 [SMILES: CCSc1oc2c([C@@H](C)Nc3cccnc3C(F)(F)F)cc(C)cc2c(=O)c1C] | 423 [M + H]⁺ |
| 131A | 8-[(1R)-1-[(2- Benzylsulfanyl-6-chloro-3- pyridyl)amino]ethyl]-3,6- dimethyl-2-phenyl-chromen- 4-one | 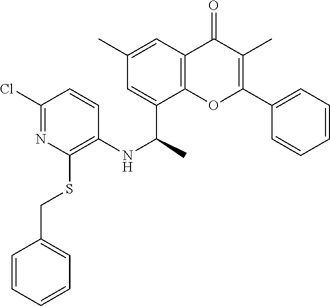 [SMILES: Cc1cc([C@@H](C)Nc2ccc(Cl)nc2SCc2ccccc2)c2oc(-c3ccccc3)c(C)c(=O)c2c1] | 527 [M + H]⁺ |
| 132A | 6-Chloro-3-[[(1R)-1-[3,6- dimethyl-4-oxo-2-(2- pyridyl)chromen-8- yl]ethyl]amino]pyridine-2- carbonitrile | 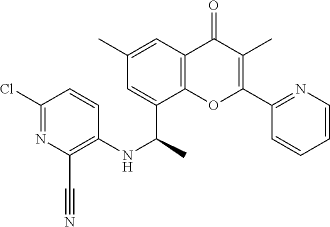 [SMILES: Cc1cc([C@@H](C)Nc2ccc(Cl)nc2C#N)c2oc(-c3ccccn3)c(C)c(=O)c2c1] | 431 [M + H]⁺ |
| 133A | tert-Butyl 6-chloro-3-[[(1R)- 1-(3,6-dimethyl-4-oxo-2- phenyl-chromen-8- yl)ethyl]amino]pyridine-2- carboxylate | 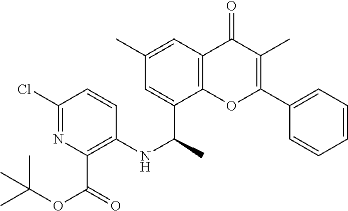 [SMILES: Cc1cc([C@@H](C)Nc2ccc(Cl)nc2C(=O)OC(C)(C)C)c2oc(-c3ccccc3)c(C)c(=O)c2c1] | 449 [M + 2H − tBu]⁺ |
| 134A | 6-[[(1R)-1-(3,6-Dimethyl-4- oxo-2-phenyl-chromen-8- yl)ethyl]amino]-2,3-difluoro- benzonitrile | 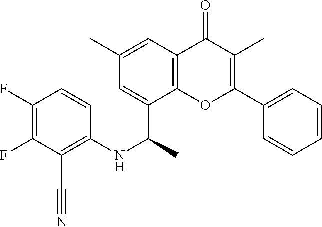 [SMILES: Cc1cc([C@@H](C)Nc2ccc(F)c(F)c2C#N)c2oc(-c3ccccc3)c(C)c(=O)c2c1] | 431 [M + H]⁺ |
| 135A | 3-[[(1R)-1-[3,6-Dimethyl-2- (2-methylindazol-5-yl)-4- oxo-chromen-8- yl]ethyl]amino]-6-methyl- pyridine-2-carbonitrile | 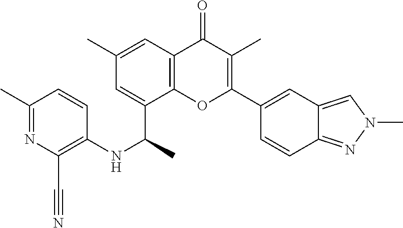 | 464 [M + H]⁺ |
| 136A | 2-[[(1R)-1-(2-Ethylsulfanyl- 3,6-dimethyl-4-oxo- chromen-8-yl)ethyl]amino]- 6-fluoro-benzonitrile | 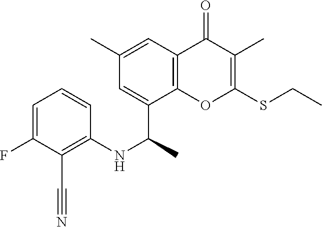 [SMILES: CCSc1oc2c([C@@H](C)Nc3cccc(F)c3C#N)cc(C)cc2c(=O)c1C] | 397 [M + H]⁺ |
| 137A | tert-Butyl 6-chloro-3-[[(1R)- 1-[2-(3,4-difluorophenyl)- 3,6-dimethyl-4-oxo- chromen-8- yl]ethyl]amino]pyridine-2- carboxylate | 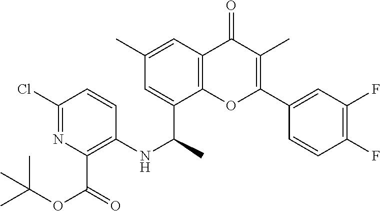 [SMILES: Cc1cc([C@@H](C)Nc2ccc(Cl)nc2C(=O)OC(C)(C)C)c2oc(-c3ccc(F)c(F)c3)c(C)c(=O)c2c1] | 485 [M + 2H − tBu]⁺ |
| 138A | 6-Chloro-3-[[(1R)-1-[2-(3,4- difluorophenyl)-3,6- dimethyl-4-oxo-chromen-8- yl]ethyl]amino]pyridine-2- carbonitrile | 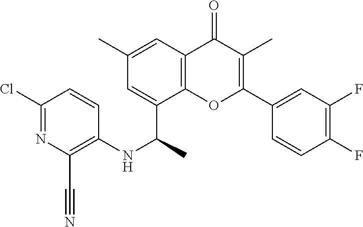 [SMILES: Cc1cc([C@@H](C)Nc2ccc(Cl)nc2C#N)c2oc(-c3ccc(F)c(F)c3)c(C)c(=O)c2c1] | 466 [M + H]⁺ |
| 139A | 6-Chloro-3-[[(1R)-1-[2-(5- fluoro-3-pyridyl)-3,6- dimethyl-4-oxo-chromen-8- yl]ethyl]amino]pyridine-2- carbonitrile | 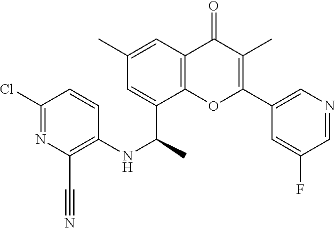 [SMILES: Cc1cc([C@@H](C)Nc2ccc(Cl)nc2C#N)c2oc(-c3cncc(F)c3)c(C)c(=O)c2c1] | 449 [M + H]⁺ |
| 140A | 2-Fluoro-6-[[(1R)-1-[2-(3- fluorophenyl)-3,6-dimethyl- 4-oxo-chromne-8- yl]ethyl]amino]benzonitrile | 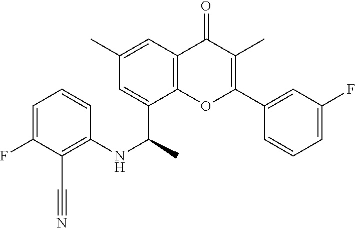 [SMILES: Cc1cc([C@@H](C)Nc2cccc(F)c2C#N)c2oc(-c3cccc(F)c3)c(C)c(=O)c2c1] | 431 [M + H]⁺ |
| 141A | 2,3-Difluoro-6-[[(1R)-1-[2- (5-fluoro-3-pyridyl)-3,6- dimethyl-4-oxo-chromen-8- yl]ethyl]amino]benzonitrile | 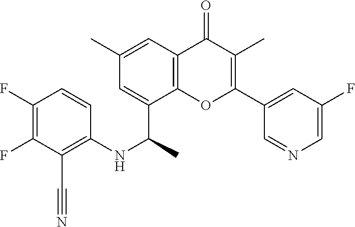 [SMILES: Cc1cc([C@@H](C)Nc2ccc(F)c(F)c2C#N)c2oc(-c3cncc(F)c3)c(C)c(=O)c2c1] | 450 [M + H]⁺ |
| 142A | tert-Butyl 3-[[(1R)-1-[3,6- dimethyl-2-(2- methylindazol-5-yl)-4-oxo- chromen-8-yl]ethyl]amino]- 6-methyl-pyridine-2- carboxylate | 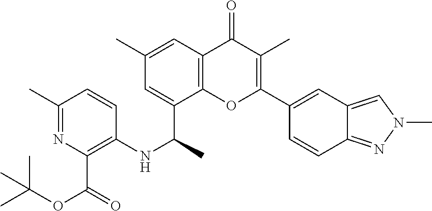 | 539 [M + H]⁺ |
| 143A | 2-[[(1R)-1-(3,6-Dimethyl-4- oxo-2-phenyl-chromen-8- yl)ethyl]amino]-6-fluoro- benzonitrile | 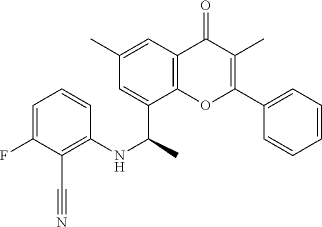 [SMILES: Cc1cc([C@@H](C)Nc2cccc(F)c2C#N)c2oc(-c3ccccc3)c(C)c(=O)c2c1] | 413 [M + H]⁺ |
| 144A | 2,3-Difluoro-6-[[(1R)-1-[2- (2-fluorophenyl)-3,6- dimethyl-4-oxo-chromen-8- yl]ethyl]amino]benzonitrile | 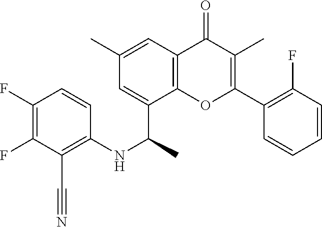 [SMILES: Cc1cc([C@@H](C)Nc2ccc(F)c(F)c2C#N)c2oc(-c3ccccc3F)c(C)c(=O)c2c1] | 449 [M + H]⁺ |
| 145A | 2-[[(1R)-1-(2-Ethylsulfanyl- 3,6-dimethyl-4-oxo- chromen-8-yl)ethyl]amino]- 5-fluoro-benzonitrile | 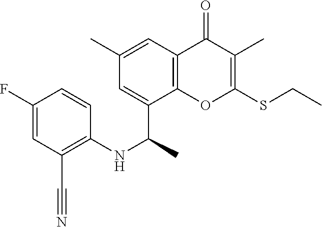 [SMILES: CCSc1oc2c([C@@H](C)Nc3ccc(F)cc3C#N)cc(C)cc2c(=O)c1C] | 397 [M + H]⁺ |
| 146A | 6-[[(1R)-1-[3,6-Dimethyl-4- oxo-2-(3-pyridyl)chromen-8- yl]ethyl]amino]-2,3-difluoro- benzonitrile | 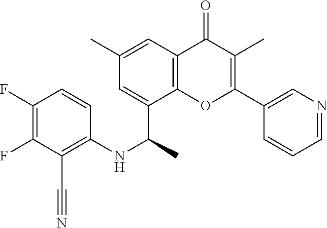 [SMILES: Cc1cc([C@@H](C)Nc2ccc(F)c(F)c2C#N)c2oc(-c3cccnc3)c(C)c(=O)c2c1] | 432 [M + H]⁺ |
| 147A | 2-Fluoro-6-[[(1R)-1-[2-(2- fluorophenyl)-3,6-dimethyl- 4-oxo-chromen-8- yl]ethyl]amino]benzonitrile | 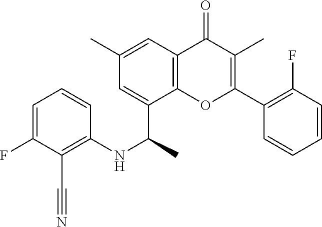 [SMILES: Cc1cc([C@@H](C)Nc2cccc(F)c2C#N)c2oc(-c3ccccc3F)c(C)c(=O)c2c1] | 431 [M + H]⁺ |
| 148A | tert-Butyl 6-chloro-3-[[(1R)- 1-[3,6-dimethyl-2-(1- methylpyrazol-4-yl)-4-oxo- chromen-8- yl]ethyl]amino]pyridine-2- carboxylate | 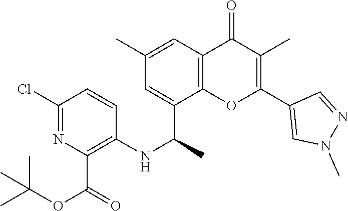 [SMILES: Cc1cc([C@@H](C)Nc2ccc(Cl)nc2C(=O)OC(C)(C)C)c2oc(-c3cnn(C)c3)c(C)c(=O)c2c1] | 507 [M − H]⁻ |
| 149A | tert-Butyl 6-chloro-3-[[(1R)- 1-[3,6-dimethyl-2-(2- methylindazol-5-yl)-4-oxo- chromen-8- yl]ethyl]amino]pyridine-2- carboxylate | 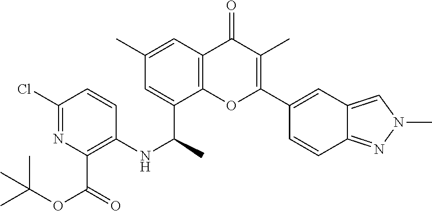 | 557 [M − H]⁻ |
| 150A | tert-Butyl 6-chloro-3-[[(1R)- 1-(3,6-dimethyl-4-oxo-2- phenyl-chromen-8- yl)ethyl]amino]pyridine-2- carboxylate | 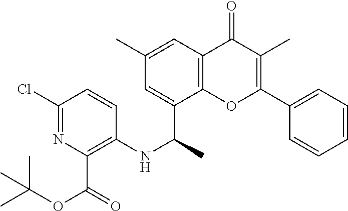 [SMILES: Cc1cc([C@@H](C)Nc2ccc(Cl)nc2C(=O)OC(C)(C)C)c2oc(-c3ccccc3)c(C)c(=O)c2c1] | 449 [M + H]⁺ |
| 151A | 3-[[(1R)-1-[2-(2- Fluorophenyl)-3,6-dimethyl- 4-oxo-chromen-8- yl]ethyl]amino]-6-methyl- pyridine-2-carbonitrile | 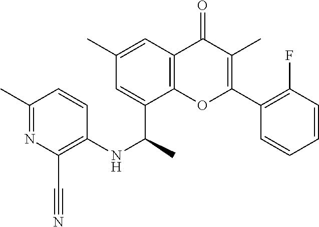 [SMILES: Cc1cc([C@@H](C)Nc2ccc(C)nc2C#N)c2oc(-c3ccccc3F)c(C)c(=O)c2c1] | 428 [M + H]⁺ |
| 152A | 2-[[(1R)-1-(3,6-Dimethyl-4- oxo-2-phenyl-chromen-8- yl)ethyl]amino]-5-fluoro- benzonitrile | 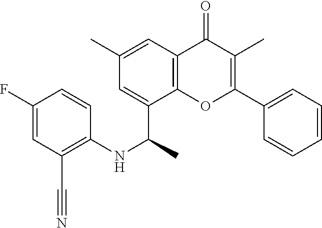 [SMILES: Cc1cc([C@@H](C)Nc2ccc(F)cc2C#N)c2oc(-c3ccccc3)c(C)c(=O)c2c1] | 413 [M + H]⁺ |
| 153A | 3-Chloro-6-[[(1R)-1-(3,6- dimethyl-4-oxo-2-phenyl- chromen-8-yl)ethyl]amino]- 2-fluoro-benzonitrile | 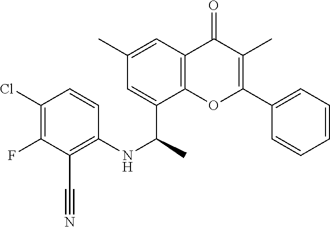 [SMILES: Cc1cc([C@@H](C)Nc2ccc(Cl)c(F)c2C#N)c2oc(-c3ccccc3)c(C)c(=O)c2c1] | 447 [M + H]⁺ |
| 154A | tert-Butyl 6-chloro-3-[[(1R)- 1-[2-[1- (difluoromethyl)pyrazol-4- yl]-3,6-dimethyl-4-oxo- chromen-8- yl]ethyl]amino]pyridine-2- carboxylate | 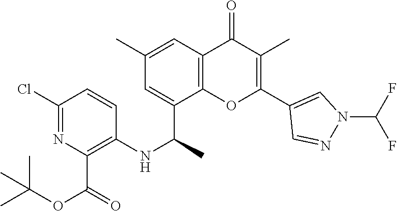 [SMILES: Cc1cc([C@@H](C)Nc2ccc(Cl)nc2C(=O)OC(C)(C)C)c2oc(-c3cnn(C(F)F)c3)c(C)c(=O)c2c1] | [M + 2H − tBu]⁺ |
| 155A | tert-Butyl 6-chloro-3-[[(1R)- 1-[3,6-dimethyl-4-oxo-2-(2- pyridyl)chromen-8- yl]ethyl]amino]pyridine-2- carboxylate | 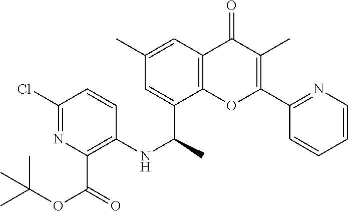 [SMILES: Cc1cc([C@@H](C)Nc2ccc(Cl)nc2C(=O)OC(C)(C)C)c2oc(-c3ccccn3)c(C)c(=O)c2c1] | 506 [M + H]⁺ |
| 156A | 6-[[(1R)-1-(3,6-Dimethyl-4- oxo-2-phenyl-chromen-8- yl)ethyl]amino]-2,3-difluoro- benzonitrile | 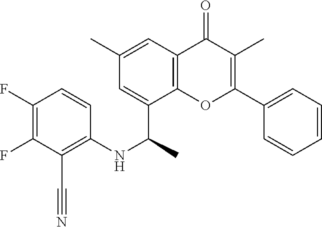 [SMILES: Cc1cc([C@@H](C)Nc2ccc(F)c(F)c2C#N)c2oc(-c3ccccc3)c(C)c(=O)c2c1] | 431 [M + H]⁺ |
| 157A | tert-Butyl 3-[[(1R)-1-(3,6- dimethyl-4-oxo-2-phenyl- chromen-8- yl)ethyl]mino]pyridine-2- carboxylate | 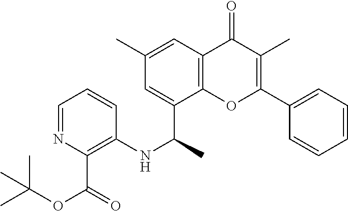 [SMILES: Cc1cc([C@@H](C)Nc2cccnc2C(=O)OC(C)(C)C)c2oc(-c3ccccc3)c(C)c(=O)c2c1] | 471 [M + H]⁺ |
| 158A | tert-Butyl 3-[[(1R)-1-(3,6- dimethyl-4-oxo-2-phenyl- chromen-8-yl)ethyl]amino]- 6-methyl-pyridine-2- carboxylate | 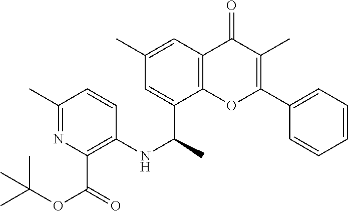 [SMILES: Cc1cc([C@@H](C)Nc2ccc(C)nc2C(=O)OC(C)(C)C)c2oc(-c3ccccc3)c(C)c(=O)c2c1] | 485 [M + H]⁺ |
| 159A | tert-Butyl 3-[[(1R)-1-(3,6- dimethyl-4-oxo-2-phenyl- chromen-8- yl)ethyl]amino]pyridine-2- carboxylate | 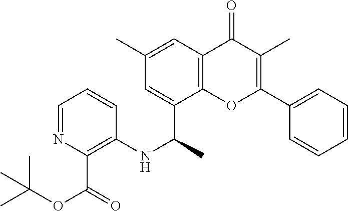 [SMILES: Cc1cc([C@@H](C)Nc2cccnc2C(=O)OC(C)(C)C)c2oc(-c3ccccc3)c(C)c(=O)c2c1] | 471 [M + H]⁺ |
| 160A | tert-Butyl 6-[[(1R)-1-(3,6- dimethyl-4-oxo-2-phenyl- chromen-8-yl)ethyl]amino]- 2,3-difluoro-benzoate | 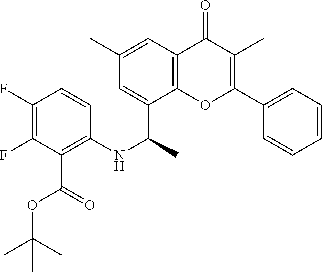 [SMILES: Cc1cc([C@@H](C)Nc2ccc(F)c(F)c2C(=O)OC(C)(C)C)c2oc(-c3ccccc3)c(C)c(=O)c2c1] | 506 [M + H]⁺ |
| 161A | 3-[[(1R)-1-[2-(5-Fluoro-3- pyridyl)-3,6-dimethyl-4-oxo- chromen-8- yl]ethyl]amino]pyridine-2- carbonitrile | 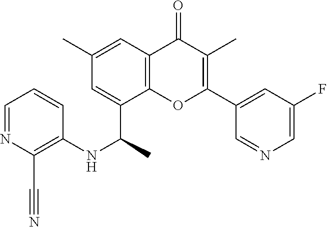 [SMILES: Cc1cc([C@@H](C)Nc2cccnc2C#N)c2oc(-c3cncc(F)c3)c(C)c(=O)c2c1] | 415 [M + H]⁺ |
| 162A | 5-Chloro-2-[[(1R)-1-[3,6- dimethyl-4-oxo-2-(3- pyridyl)chromen-8- yl]ethyl]amino]benzonitrile | 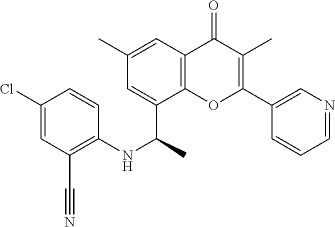 [SMILES: Cc1cc([C@@H](C)Nc2ccc(Cl)cc2C#N)c2oc(-c3cccnc3)c(C)c(=O)c2c1] | 430 [M + H]⁺ |
| 163A | 8-[(1R)-1-(2- Bromoanilino)ethyl]-3,6- dimethyl-2-phenyl- chromen-4-one | 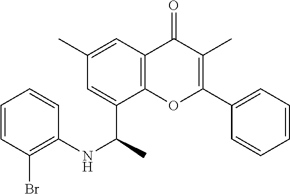 [SMILES: Cc1cc([C@@H](C)Nc2ccccc2Br)c2oc(-c3ccccc3)c(C)c(=O)c2c1] | 450 [M + H]⁺ |
| 164A | 3-[[(1R)-1-[2-(3,4- Difluorophenyl)-3,6- dimethyl-4-oxo-chromen-8- yl]ethyl]amino]-6-methyl- pyridine-2-carbonitrile | 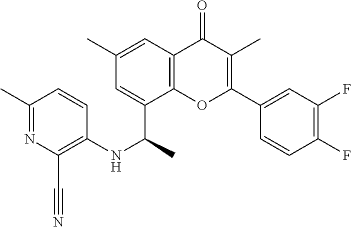 [SMILES: Cc1cc([C@@H](C)Nc2ccc(C)nc2C#N)c2oc(-c3ccc(F)c(F)c3)c(C)c(=O)c2c1] | 446 [M + H]⁺ |
| 165A | 3-[[(1R)-1-[3,6-Dimethyl-2- (1-methylpyrazol-4-yl)-4- oxo-chromen-8- yl]ethyl]amino]pyridine-2- carbonitrile | 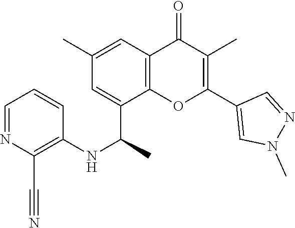 [SMILES: Cc1cc([C@@H](C)Nc2cccnc2C#N)c2oc(-c3cnn(C)c3)c(C)c(=O)c2c1] | 400 [M + H]⁺ |
| 166A | 3-[[(1R)-1-[2-(2-Fluoro-3- pyridyl)-3,6-dimethyl-4-oxo- chromen-8- yl]ethyl]amino]pyridine-2- carbonitrile | 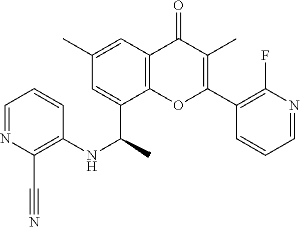 [SMILES: Cc1cc([C@@H](C)Nc2cccnc2C#N)c2oc(-c3cccnc3F)c(C)c(=O)c2c1] | 415 [M + H]⁺ |
| 167A | 3-[[(1R)-1-[2-(2- Fluorophenyl)-3,6-dimethyl- 4-oxo-chromen-8- yl]ethyl]amino]pyridine-2- carbonitrile | 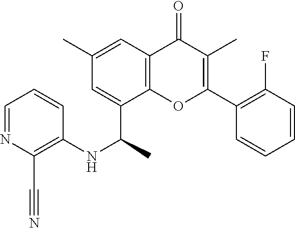 [SMILES: Cc1cc([C@@H](C)Nc2cccnc2C#N)c2oc(-c3ccccc3F)c(C)c(=O)c2c1] | 414 [M + H]⁺ |
| 168A | 2-[[(1R)-1-[2-(2,3- Difluorophenyl)-3,6- dimethyl-4-oxo-chromen-8- yl]ethyl]amino]-6-fluoro- benzonitrile | 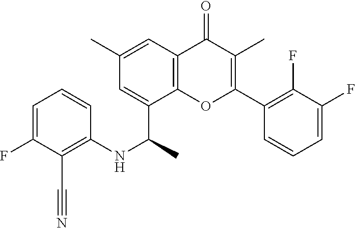 [SMILES: Cc1cc([C@@H](C)Nc2cccc(F)c2C#N)c2oc(-c3cccc(F)c3F)c(C)c(=O)c2c1] | 449 [M + H]+ |
| 169A | 3-[[(1R)-1-[3,6-Dimethyl-2- (2-methylindazol-5-yl)-4- oxo-chromen-8- yl]ethyl]amino]pyridine-2- carbonitrile | 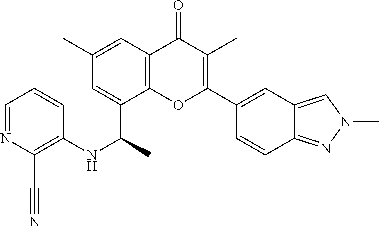 [SMILES: Cc1cc([C@@H](C)Nc2cccnc2C#N)c2oc(-c3ccc4nn(C)cc4c3)c(C)c(=O)c2c1] | 450 [M + H]⁺ |
| 170A | Methyl 2-[[(1R)-1-(3,6- dimethyl-4-oxo-2-phenyl- chromen-8-yl)ethyl]amino]- 6-fluoro-benzoate | 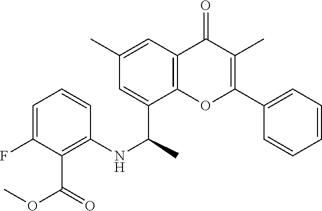 [SMILES: COC(=O)c1c(F)cccc1N[C@H](C)c1cc(C)cc2c(=O)c(C)c(-c3ccccc3)oc12] | 446 [M + H]⁺ |
| 171A | 3-[[(1R)-1-[2-[6- (Difluoromethyl)-2-pyridyl]- 3,6-dimethyl-4-oxo- chromen-8-yl]ethyl]amino]- 6-methyl-pyridine-2- carbonitrile | 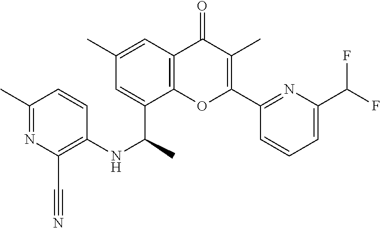 [SMILES: Cc1cc([C@@H](C)Nc2ccc(C)nc2C#N)c2oc(-c3cccc(C(F)F)n3)c(C)c(=O)c2c1] | 461 [M + H]⁺ |
| 172A | tert-Butyl 3-[[(1R)-1-[2-(3,4- difluorophenyl)-3,6- dimethyl-4-oxo-chromen-8- yl]ethyl]amino]-6-methyl- pyridine-2-carboxylate | 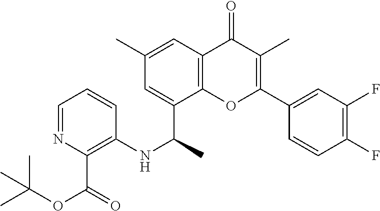 | 521 [M + H]⁺ |
| 173A | tert-Butyl 3-[[(1R)-1-[3,6- dimethyl-4-oxo-2-(3- pyridyl)chromen-8- yl]ethyl]amino]pyridine-2- carboxylate | 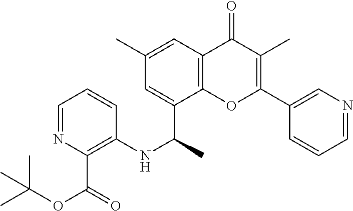 [SMILES: Cc1cc([C@@H](C)Nc2cccnc2C(=O)OC(C)(C)C)c2oc(-c3cccnc3)c(C)c(=O)c2c1] | 472 [M + H]⁺ |
| 174A | 2-[[(1R)-1-[2-(2,4- Difluorophenyl)-3,6- dimethyl-4-oxo-chromen-8- yl]ethyl]amino]-6-fluoro- benzonitrile | 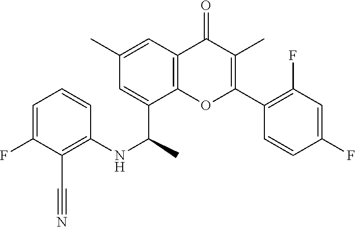 [SMILES: Cc1cc([C@@H](C)Nc2cccc(F)c2C#N)c2oc(-c3ccc(F)cc3F)c(C)c(=O)c2c1] | 449 [M + H]⁺ |
| 175A | 3-[[(1R)-1-[2-[6- (Difluoromethyl)-2-pyridyl]- 3,6-dimethyl-4-oxo- chromen-8- yl]ethyl]amino]pyridine-2- carbonitrile | 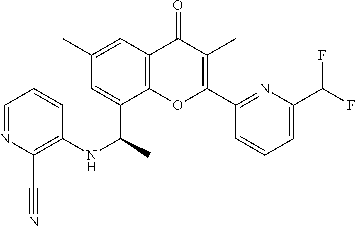 [SMILES: Cc1cc([C@@H](C)Nc2cccnc2C#N)c2oc(-c3cccc(C(F)F)n3)c(C)c(=O)c2c1] | 447 [M + H]⁺ |
| 176A | tert-Butyl 7-[[(1R)-1-[3,6- dimethyl-4-oxo-2-(3- pyridyl)chromen-8- yl]ethyl]amino]-1-oxo- isoindoline-2-carboxylate | 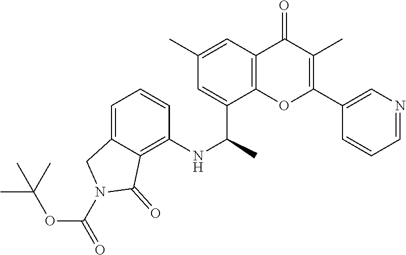 [SMILES: Cc1cc([C@@H](C)Nc2cccc3c2C(=O)N(C(=O)OC(C)(C)C)C3)c2oc(-c3cccnc3)c(C)c(=O)c2c1] | 526 [M + H]⁺ |
| 177A | 2-Chloro-6-[[(1R)-1-(3,6- dimethyl-4-oxo-2-phenyl- chromen-8- yl)ethyl]amino]benzonitrile | 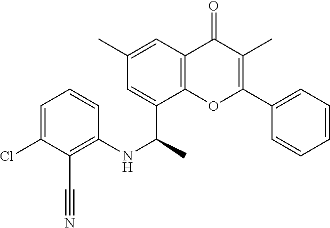 [SMILES: Cc1cc([C@@H](C)Nc2cccc(Cl)c2C#N)c2oc(-c3ccccc3)c(C)c(=O)c2c1] | 429 [M + H]⁺ |
| 178A | 8-[(1R)-1-(2- Bromoanilino)ethyl]-2- ethylsulfanyl-3,6-dimethyl- chromen-4-one | 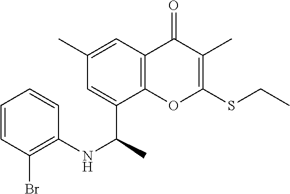 [SMILES: CCSc1oc2c([C@@H](C)Nc3ccccc3Br)cc(C)cc2c(=O)c1C] | 434 [M + H]⁺ |
| 179A | 6-Chloro-3-[[(1R)-1-[3,6- dimethyl-2-(1- methylpyrazol-4-yl)-4-oxo- chromen-8- yl]ethyl]amino]pyridine-2- carbonitrile | 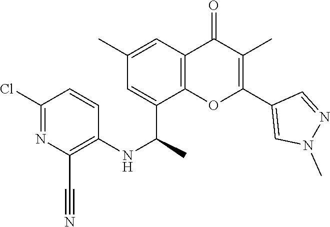 [SMILES: Cc1cc([C@@H](C)Nc2ccc(Cl)nc2C#N)c2oc(-c3cnn(C)c3)c(C)c(=O)c2c1] | 434 [M + H]⁺ |
| 180A | 6-Chloro-3-[[(1R)-1-[2-(6- fluoro-2-pyridyl)-3,6- dimethyl-4-oxo-chromen-8- yl]ethyl]amino]pyridine-2- carbonitrile | 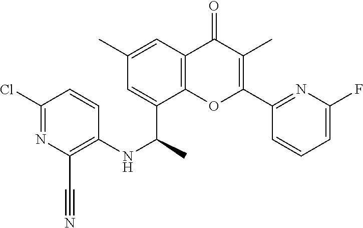 [SMILES: Cc1cc([C@@H](C)Nc2ccc(Cl)nc2C#N)c2oc(-c3cccc(F)n3)c(C)c(=O)c2c1] | 449 [M + H]⁺ |
| 181A | 6-Chloro-3-[[(1R)-1-[3,6- dimethyl-4-oxo-2-(3- pyridyl)chromen-8- yl]ethyl]amino]pyridine-2- carbonitrile | 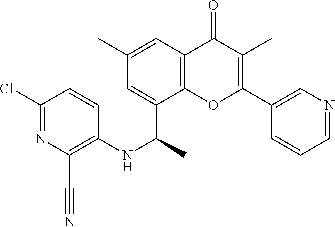 [SMILES: Cc1cc([C@@H](C)Nc2ccc(Cl)nc2C#N)c2oc(-c3cccnc3)c(C)c(=O)c2c1] | 431 [M + H]⁺ |
| 182A | 6-Chloro-3-[[(1R)-1-[2-(2- fluoro-3-pyridyl)-3,6- dimethyl-4-oxo-chromen-8- yl]ethyl]amino]pyridine-2- carbonitrile | 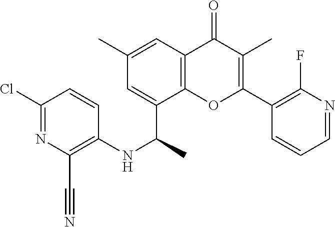 [SMILES: Cc1cc([C@@H](C)Nc2ccc(Cl)nc2C#N)c2oc(-c3cccnc3F)c(C)c(=O)c2c1] | 449 [M + H]⁺ |
| 183A | 8-[(1R)-1-[(6-Chloro-2- ethylsulfanyl-3- pyridyl)amino]ethyl]-3,6- dimethyl-2-(2- methylindazol-5-yl)chromen- 4-one | 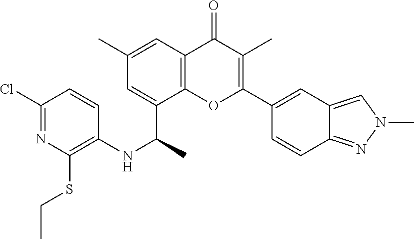 | 581 [M + H]⁺ |
| 184A | 6-Chloro-3-[[(1R)-1-[2-(3- fluoro-2-pyridyl)-3,6- dimethyl-4-oxo-chromen-8- yl]ethyl]amino]pyridine-2- carbonitrile | 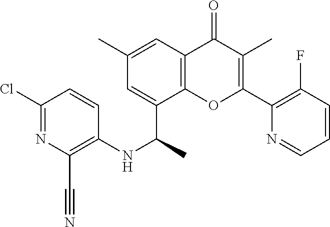 [SMILES: Cc1cc([C@@H](C)Nc2ccc(Cl)nc2C#N)c2oc(-c3ncccc3F)c(C)c(=O)c2c1] | 449 [M + H]⁺ |
| 185A | 6-Chloro-3-[[(1R)-1-[2-(2,5- difluoro-3-pyridyl)-3,6- dimethyl-4-oxo-chromen-8- yl]ethyl]amino]pyridine-2- carbonitrile | 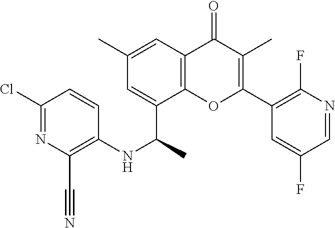 [SMILES: Cc1cc([C@@H](C)Nc2ccc(Cl)nc2C#N)c2oc(-c3cc(F)cnc3F)c(C)c(=O)c2c1] | 467 [M + H]⁺ |
| 186A | 6-Chloro-3-[[(1R)-1-[2-(6- methoxy-2-pyridyl)-3,6- dimethyl-4-oxo-chromen-8- yl]ethyl]amino]pyridine-2- carbonitrile | 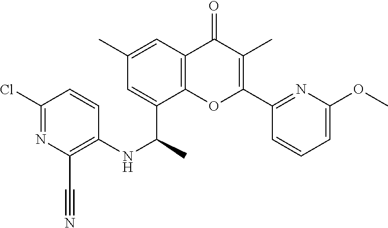 [SMILES: COc1cccc(-c2oc3c([C@@H](C)Nc4ccc(Cl)nc4C#N)cc(C)cc3c(=O)c2C)n1] | 461 [M + H]⁺ |
| 187A | tert-Butyl 6-chloro-3-[[(1R)- 1-[3-methyl-2-(1- methylpyrazol-4-yl)-4-oxo- 6-(trifluoromethyl)chromen- 8-yl]ethyl]amino]pyridine-2- carboxylate | 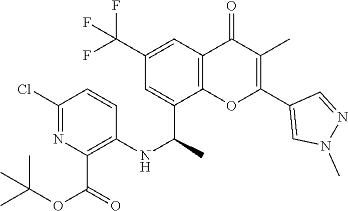 [SMILES: Cc1c(-c2cnn(C)c2)oc2c([C@@H](C)Nc3ccc(Cl)nc3C(=O)OC(C)(C)C)cc(C(F)(F)F)cc2c1=O] | 507 [M − tBu]⁺ |
| 188A | 6-Chloro-3-[[(1R)-1-[3- methyl-4-oxo-2-(2-pyridyl)- 6-(trifluoromethyl)chromen- 8-yl]ethyl]amino]pyridine-2- carbonitrile | 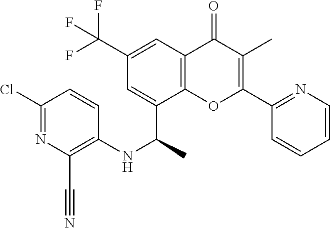 [SMILES: Cc1c(-c2ccccn2)oc2c([C@@H](C)Nc3ccc(Cl)nc3C#N)cc(C(F)(F)F)cc2c1=O] | 485 [M + H]⁺ |
| 189A | 6-Chloro-3-[[(1R)-1-[6- methyl-2-(1-methylpyrazol- 4-yl)-4-oxo-chromen-8- yl]ethyl]amino]pyridine-2- carbonitrile | 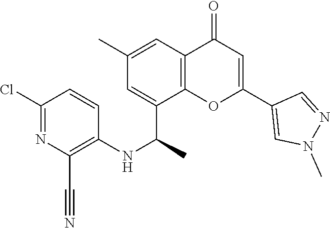 [SMILES: Cc1cc([C@@H](C)Nc2ccc(Cl)nc2C#N)c2oc(-c3cnn(C)c3)cc(=O)c2c1] | 420 [M + H]⁺ |
| 190A | 3-[[(1R)-1-[2-(5-Fluoro-3- pyridyl)-3-methyl-4-oxo-6- (trifluoromethyl)chromen-8- yl]ethyl]amino]pyridine-2- carbonitrile | 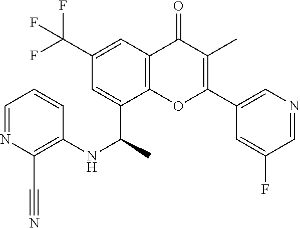 [SMILES: Cc1c(-c2cncc(F)c2)oc2c([C@@H](C)Nc3cccnc3C#N)cc(C(F)(F)F)cc2c1=O] | 469 [M + H]⁺ |
| 191A | 6-Chloro-3-[[(1R)-1-[2-(2,6- difluorophenyl)-3,6- dimethyl-4-oxo-chromen-8- yl]ethyl]amino]pyridine-2- carbonitrile | 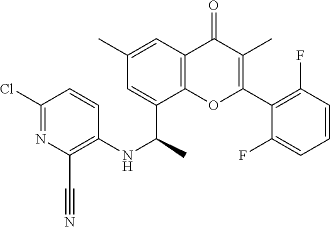 [SMILES: Cc1cc([C@@H](C)Nc2ccc(Cl)nc2C#N)c2oc(-c3c(F)cccc3F)c(C)c(=O)c2c1] | 466 [M + H]⁺ |
| 192A | 3-[[(1R)-1-[3-Methyl-4-oxo- 2-(2-pyridyl)-6- (trifluoromethyl)chromen-8- yl]ethyl]amino]pyridine-2- carbonitrile | 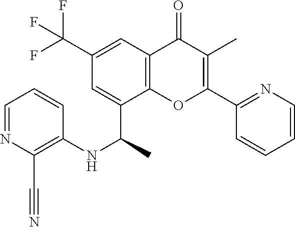 [SMILES: Cc1c(-c2ccccn2)oc2c([C@@H](C)Nc3cccnc3C#N)cc(C(F)(F)F)cc2c1=O] | 451 [M + H]⁺ |
| 193A | tert-Butyl 6-chloro-3-[[(1R)- 1-[2-(6-fluoro-2-pyridyl)- 3,6-dimethyl-4-oxo- chromen-8- yl]ethyl]amino]pyridine-2- carboxylate | 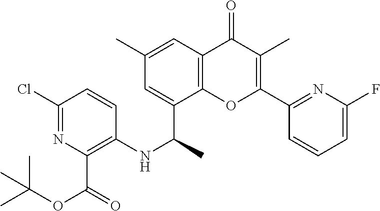 [SMILES: Cc1cc([C@@H](C)Nc2ccc(Cl)nc2C(=O)OC(C)(C)C)c2oc(-c3cccc(F)n3)c(C)c(=O)c2c1] | 468 [M + 2H − tBu]+ |
| 194A | 6-[[(1R)-1-(3,6-Dimethyl-4- oxo-2-pyrimidin-5-yl- chromen-8-yl)ethyl]amino]- 2,3-difluoro-benzonitrile | 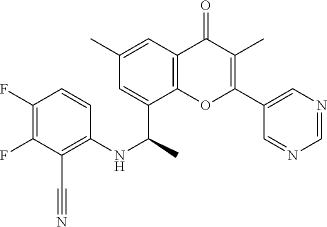 [SMILES: Cc1cc([C@@H](C)Nc2ccc(F)c(F)c2C#N)c2oc(-c3cncnc3)c(C)c(=O)c2c1] | 433 [M + H]⁺ |
| 202C | 6-Chloro-3-[[(1R)-1-[2-(2,5- dimethylpyrazol-3-yl)-3,6- dimethyl-4-oxo-chromen-8- yl]ethyl]amino]pyridine-2- carbonitrile | 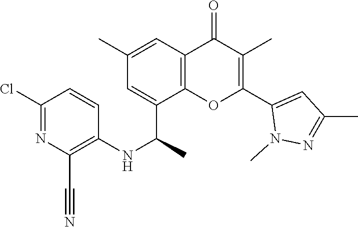 [SMILES: Cc1cc([C@@H](C)Nc2ccc(Cl)nc2C#N)c2oc(-c3cc(C)nn3C)c(C)c(=O)c2c1] | 448 [M + H]⁺ |
| 203C | 3-[[(1R)-1-[2-[1-[2-[tert- Butyl(dimethyl)silyl]oxy- ethyl]pyrazol-4-yl]-3,6- dimethyl-4-oxo- chromen-8- yl]ethyl]amino]-6-chloro- pyridine-2-carbonitrile | 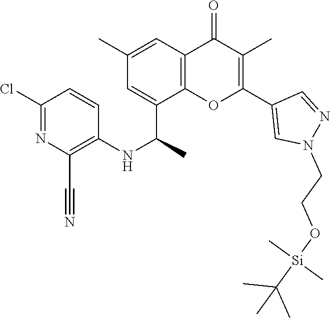 [SMILES: Cc1cc([C@@H](C)Nc2ccc(Cl)nc2C#N)c2oc(-c3cnn(CCO[Si](C)(C)C(C)(C)C)c3)c(C)c(=O)c2c1] | 578 [M + H]⁺ |
| 204C | 6-Chloro-3-[[(1R)-1-(3,6- dimethyl-4-oxo-2-pyrazin-2- yl-chromen-8- yl)ethyl]amino]pyridine-2- carbonitrile | 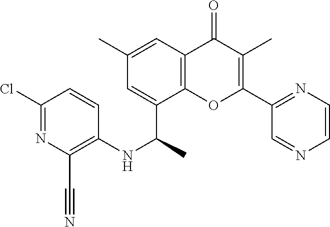 [SMILES: Cc1cc([C@@H](C)Nc2ccc(Cl)nc2C#N)c2oc(-c3cnccn3)c(C)c(=O)c2c1] | 432 [M + H]⁺ |
| 205C | 6-Chloro-3-[[(1R)-1-[3,6- dimethyl-2-(2-methyltriazol- 4-yl)-4-oxo-chromen-8- yl]ethyl]amino]pyridine-2- carbonitrile | 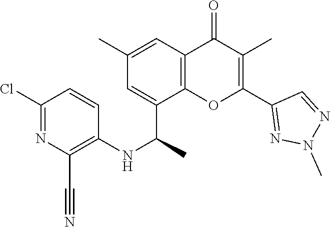 [SMILES: Cc1cc([C@@H](C)Nc2ccc(Cl)nc2C#N)c2oc(-c3cnn(C)n3)c(C)c(=O)c2c1] | 435 [M + H]⁺ |
| 206C | 6-Chloro-3-[[(1R)-1-[2-(3- fluoro-1-methyl-pyrazol-4- yl)-3,6-dimethyl-4-oxo- chromen-8- yl]ethyl]amino]pyridine-2- carbonitrile | 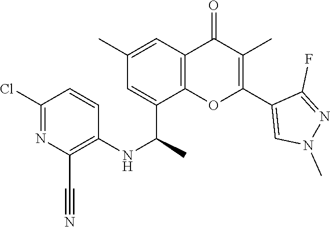 [SMILES: Cc1cc([C@@H](C)Nc2ccc(Cl)nc2C#N)c2oc(-c3cn(C)nc3F)c(C)c(=O)c2c1] | 452 [M + H]⁺ |
| 207C | N-tert-Butyl-6-chloro-3- [[(1R)-1-[6-methyl-2-(1- methyl-6-oxo-3-pyridyl)-4- oxo-chromen-8- yl]ethyl]amino]pyridine-2- sulfonamide | 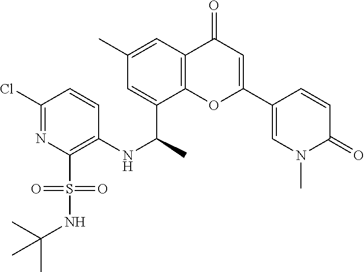 [SMILES: Cc1cc([C@@H](C)Nc2ccc(Cl)nc2S(=O)(=O)NC(C)(C)C)c2oc(-c3ccc(=O)n(C)c3)cc(=O)c2c1] | 557 [M + H]⁺ |
| 208C | 6-Chloro-3-[[(1R)-1-(6- methyl-3-oxazol-4-yl-4-oxo- 2-phenyl-chromen-8- yl)ethyl]amino]pyridine-2- carbonitrile | 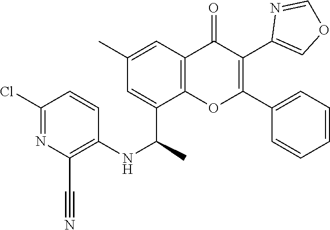 [SMILES: Cc1cc([C@@H](C)Nc2ccc(Cl)nc2C#N)c2oc(-c3ccccc3)c(-c3cocn3)c(=O)c2c1] | 483 [M + H]⁺ |
| 209C¹ | 8-[(1R)-1-[(2-Benzoyloxy-3- pyridyl)amino]ethyl]-3,6- dimethyl-2-(3- pyridyl)chromen-4-one | 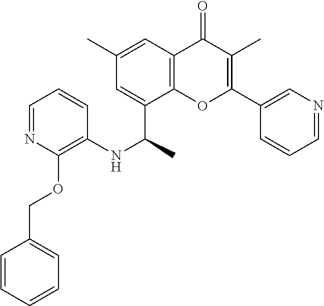 [SMILES: Cc1cc([C@@H](C)Nc2cccnc2OCc2ccccc2)c2oc(-c3cccnc3)c(C)c(=O)c2c1] | 478 [M + H]⁺ |
| 210C | tert-Butyl 6-[[(1R)-1-[3,6- dimethyl-2-(2- methylindazol-5-yl)-4-oxo- chromen-8-yl]ethyl]amino]- 2,3-difluoro-benzoate | 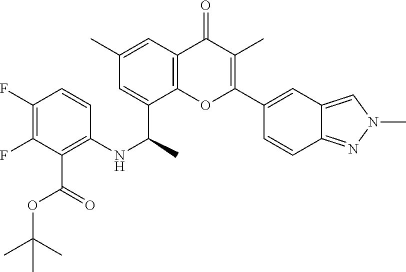 [SMILES: Cc1cc([C@@H](C)Nc2ccc(F)c(F)c2C(=O)OC(C)(C)C)c2oc(-c3ccc4nn(C)cc4c3)c(C)c(=O)c2c1] | 560 [M + H]⁺ |
| 211C | 6-Chloro-3-[[(1R)-1-[6- methyl-2-(1-methylpyrazol- 4-yl)-3-oxazol-4-yl-4-oxo- chromen-8- yl]ethyl]amino]pyridine-2- carbonitrile | 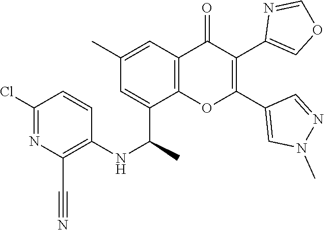 [SMILES: Cc1cc([C@@H](C)Nc2ccc(Cl)nc2C#N)c2oc(-c3cnn(C)c3)c(-c3cocn3)c(=O)c2c1] | 487 [M + H]⁺ |
| 212C | N-tert-Butyl-6-chloro-3- [[(1R)-1-[3,6-dimethyl-2-(1- methyl-2-oxo-4-pyridyl)-4- oxo-chromen-8- yl]ethyl]amino]pyridine-2- sulfonamide | 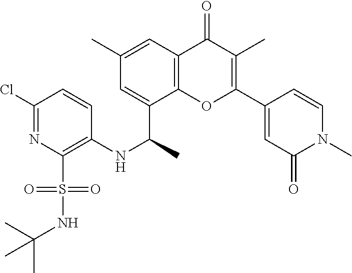 [SMILES: Cc1cc([C@@H](C)Nc2ccc(Cl)nc2S(=O)(=O)NC(C)(C)C)c2oc(-c3ccn(C)c(=O)c3)c(C)c(=O)c2c1] | 571 [M + H]⁺ |
| 213C | N-tert-Butyl-6-chloro-3- [[(1R)-1-[3,6-dimethyl-2-(1- methyl-6-oxo-3-pyridyl)-4- oxo-chromen-8- yl]ethyl]amino]pyridine-2- sulfonamide | 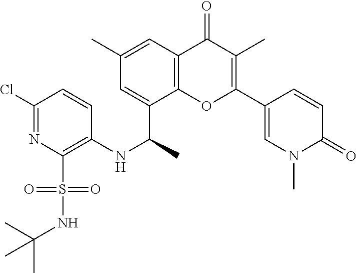 [SMILES: Cc1cc([C@@H](C)Nc2ccc(Cl)nc2S(=O)(=O)NC(C)(C)C)c2oc(-c3ccc(=O)n(C)c3)c(C)c(=O)c2c1] | 571 [M + H]⁺ |
| 214C | 6-Chloro-3-[[(1R)-1-[2-(3,6- difluoro-2-pyridyl)-3,6- dimethyl-4-oxo-chromen-8- yl]ethyl]amino]pyridine-2- carbonitrile | 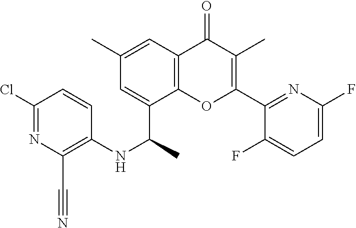 [SMILES: Cc1cc([C@@H](C)Nc2ccc(Cl)nc2C#N)c2oc(-c3nc(F)ccc3F)c(C)c(=O)c2c1] | 467 [M + H]⁺ |
| 215C | 6-Chloro-3-[[(1R)-1-[2-(2,4- difluoro-3-pyridyl)-3,6- dimethyl-4-oxo-chromen-8- yl]ethyl]amino]pyridine-2- carbonitrile | 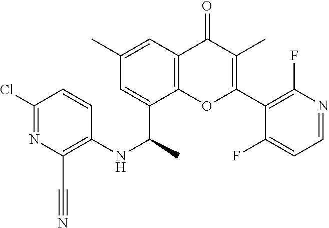 [SMILES: Cc1cc([C@@H](C)Nc2ccc(Cl)nc2C#N)c2oc(-c3c(F)ccnc3F)c(C)c(=O)c2c1] | 467 [M + H]⁺ |
| 216C | 6-Chloro-3-[[(1R)-1-[2-(4- fluoro-3-pyridyl)-3,6- dimethyl-4-oxo-chromen-8- yl]ethyl]amino]pyridine-2- carbonitrile | 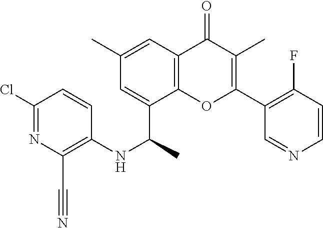 [SMILES: Cc1cc([C@@H](C)Nc2ccc(Cl)nc2C#N)c2oc(-c3cnccc3F)c(C)c(=O)c2c1] | 449 [M + H]⁺ |
| 217C | 6-Chloro-3-[[(1R)-1-[2-(5- fluoro-2-pyridyl)-3,6- dimethyl-4-oxo-chromen-8- yl]ethyl]amino]pyridine-2- carbonitrile | 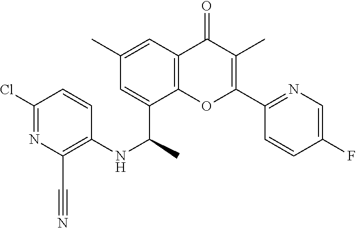 [SMILES: Cc1cc([C@@H](C)Nc2ccc(Cl)nc2C#N)c2oc(-c3ccc(F)cn3)c(C)c(=O)c2c1] | 449 [M + H]⁺ |
| 218C | 6-Chloro-3-[[(1R)-1-(3,6- dimethyl-4-oxo-2-pyrimidin- 2-yl-chromen-8- yl)ethyl]amino]pyridine-2- carbonitrile | 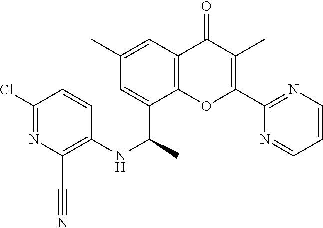 [SMILES: Cc1cc([C@@H](C)Nc2ccc(Cl)nc2C#N)c2oc(-c3ncccn3)c(C)c(=O)c2c1] | 432 [M + H]⁺ |
| 219C | 6-Chloro-3-[[(1R)-1-[2-(6- methoxy-2-pyridyl)-3,6- dimethyl-4-oxo-chromen-8- yl]ethyl]amino]pyridine-2- carbonitrile | 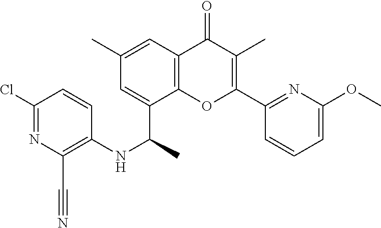 [SMILES: COc1cccc(-c2oc3c([C@@H](C)Nc4ccc(Cl)nc4C#N)cc(C)cc3c(=O)c2C)n1] | 461 [M + H]⁺ |
| 220C | 6-Chloro-3-[[(1R)-1-[6- methyl-4-oxo-2-(2- pyridyl)chromen-8- yl]ethyl]amino]pyridine-2- carbonitrile | 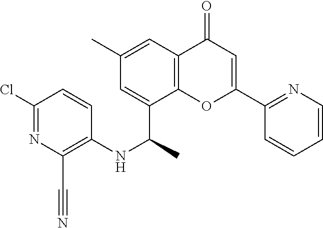 [SMILES: Cc1cc([C@@H](C)Nc2ccc(Cl)nc2C#N)c2oc(-c3ccccn3)cc(=O)c2c1] | 417 [M + H]⁺ |
| 221C | 6-Chloro-3-[[(1R)-1-[3,6- dimethyl-2-(1- methylindazol-5-yl)-4-oxo- chromen-8- yl]ethyl]amino]pyridine-2- carbonitrile | 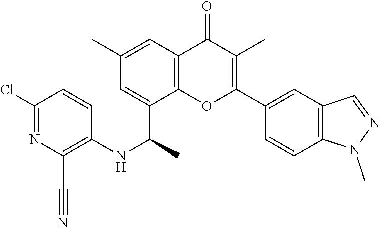 [SMILES: Cc1cc([C@@H](C)Nc2ccc(Cl)nc2C#N)c2oc(-c3ccc4c(cnn4C)c3)c(C)c(=O)c2c1] | 484 [M + H]⁺ |
| 222C | 6-[[(1R)-1-[3,6-Dimethyl-2- (2-methylindazol-5-yl)-4- oxo-chromen-8- yl]ethyl]amino]-2,3-difluoro- benzonitrile | 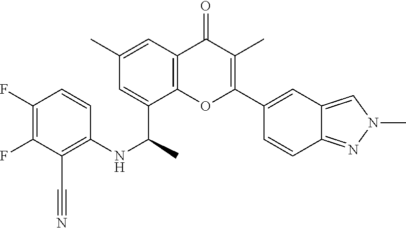 [SMILES: Cc1cc([C@@H](C)Nc2ccc(F)c(F)c2C#N)c2oc(-c3ccc4nn(C)cc4c3)c(C)c(=O)c2c1] | 485 [M + H]⁺ |
| 223C | tert-Butyl 6-chloro-3-[[(1R)- 1-[2-(2,4-difluorophenyl)- 3,6-dimethyl-4-oxo- chromen-8- yl]ethyl]amino]pyridine-2- carboxylate | 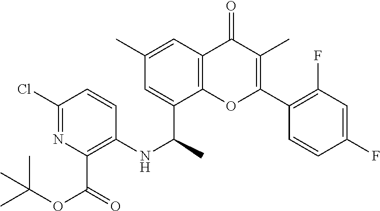 [SMILES: Cc1cc([C@@H](C)Nc2ccc(Cl)nc2C(=O)OC(C)(C)C)c2oc(-c3ccc(F)cc3F)c(C)c(=O)c2c1] | 485 [MH − tBu]⁺ |
| 224C | tert-Butyl 6-chloro-3-[[(1R)- 1-[2-(2,3-difluorophenyl)- 3,6-dimethyl-4-oxo- chromen-8- yl]ethyl]amino]pyridine-2- carboxylate | 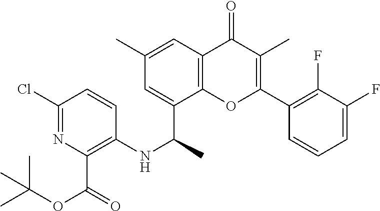 [SMILES: Cc1cc([C@@H](C)Nc2ccc(Cl)nc2C(=O)OC(C)(C)C)c2oc(-c3cccc(F)c3F)c(C)c(=O)c2c1] | 485 [MH − tBu]⁺ |
| 225C¹ | tert-Butyl 4-[[(1R)-1-[3,6- dimethyl-4-oxo-2-(3- pyridyl)chromen-8- yl]ethyl]amino]indole-1- carboxylate | 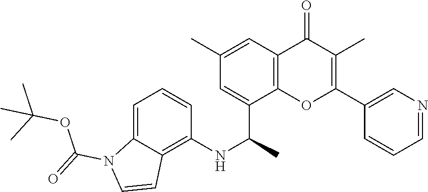 [SMILES: Cc1cc([C@@H](C)Nc2cccc3c2ccn3C(=O)OC(C)(C)C)c2oc(-c3cccnc3)c(C)c(=O)c2c1] | 510 [M + H]⁺ |
| 226C | 8-[(1R)-1-[2-(1,3-Dioxolan- 2-yl)anilino]ethyl]-3,6- dimethyl-2-phenyl- chromen-4-one | 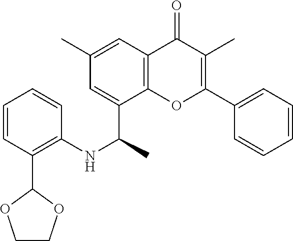 [SMILES: Cc1cc([C@@H](C)Nc2ccccc2C2OCCO2)c2oc(-c3ccccc3)c(C)c(=O)c2c1] | 442 [M + H]⁺ |
| 227C¹ | tert-Butyl 6-[[(1R)-1-[3,6- dimethyl-4-oxo-2-(3- pyridyl)chromen-8- yl]ethyl]amino]-7-methyl- indole-1-carboxylate | 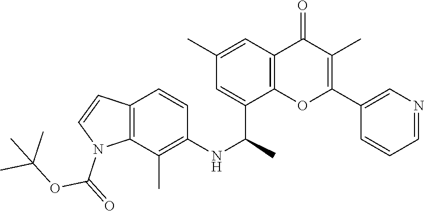 [SMILES: Cc1cc([C@@H](C)Nc2ccc3ccn(C(=O)OC(C)(C)C)c3c2C)c2oc(-c3cccnc3)c(C)c(=O)c2c1] | 524 [M + H]⁺ |
| 228C | 6-Chloro-3-[[(1R)-1-[3,6- dimethyl-2-(2- methylpyrazolo[3,4- b]pyridin-5-yl)-4-oxo- chromen-8- yl]ethyl]amino]pyridine-2- carbonitrile | 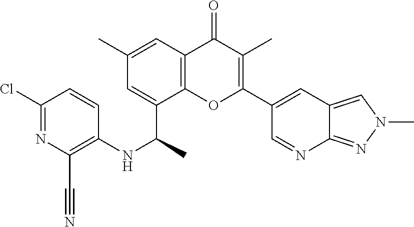 [SMILES: Cc1cc([C@@H](C)Nc2ccc(Cl)nc2C#N)c2oc(-c3cnc4nn(C)cc4c3)c(C)c(=O)c2c1] | 485 [M + H]⁺ |
| 229C | tert-Butyl 6-chloro-3-[[(1R)- 1-[3,6-dimethyl-2-(2- methylpyrazolo[3,4- b]pyridin-5-yl)-4-oxo- chromen-8- yl]ethyl]amino]pyridine-2- carboxylate | 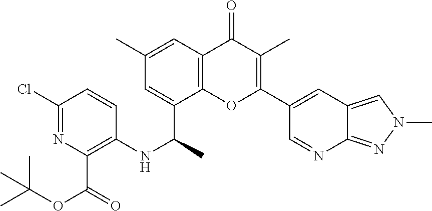 [SMILES: Cc1cc([C@@H](C)Nc2ccc(Cl)nc2C(=O)OC(C)(C)C)c2oc(-c3cnc4nn(C)cc4c3)c(C)c(=O)c2c1] | 504 [M − ^{t}Bu]+ |
| 230C² | 8-[(1R)-1-[(2-Chloro-3- pyridyl)amino]ethyl]-6- methyl-2-(3- pyridyl)chromen-4-one | 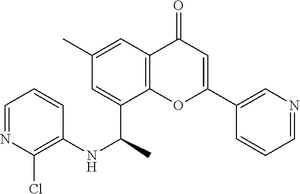 [SMILES: Cc1cc([C@@H](C)Nc2cccnc2Cl)c2oc(-c3cccnc3)cc(=O)c2c1] | 392 [M + H]⁺ |
| 231C | 6-Chloro-3-[[(1R)-1-[2-(4- fluoro-2-pyridyl)-3,6- dimethyl-4-oxo-chromen-8- yl]ethyl]amino]pyridine-2- carbonitrile | 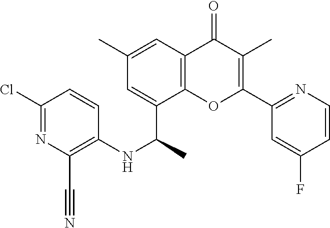 [SMILES: Cc1cc([C@@H](C)Nc2ccc(Cl)nc2C#N)c2oc(-c3cc(F)ccn3)c(C)c(=O)c2c1] | 449 [M + H]⁺ |
| 232C | 6-Chloro-3-[[(1R)-1-[2-(6,7- dihydro-4H-pyrazolo[5,1- c][1,4]oxazin-3-yl)-3,6- dimethyl-4-oxo-chromen-8- yl]ethyl]amino]pyridine-2- carbonitrile | 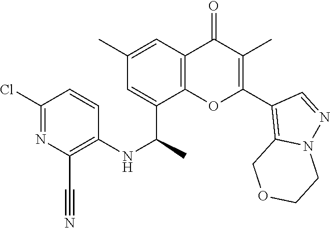 [SMILES: Cc1cc([C@@H](C)Nc2ccc(Cl)nc2C#N)c2oc(-c3cnn4c3COCC4)c(C)c(=O)c2c1] | 476 [M + H]⁺ |
| 233C | 8-[(1R)-1-[[2-[4-[tert- Butyl(dimethyl)silyl]oxy-1- piperidyl]-6-chloro-3- pyridyl]amino]ethyl]-3,6- dimethyl-2-(3- pyridyl)chromen-4-one | 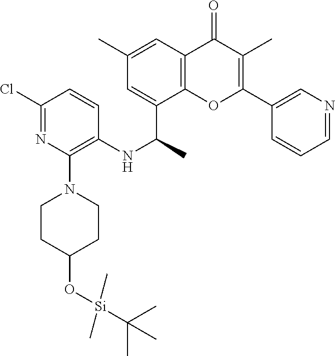 [SMILES: Cc1cc([C@@H](C)Nc2ccc(Cl)nc2N2CCC(O[Si](C)(C)C(C)(C)C)CC2)c2oc(-c3cccnc3)c(C)c(=O)c2c1] | 619 [M + H]⁺ |
| 234C | 6-Chloro-3-[[(1R)-1-[3,6- dimethyl-2-[1-(oxetan-3- yl)pyrazol-4-yl]-4-oxo- chromen-8- yl]ethyl]amino]pyridine-2- carbonitrile | 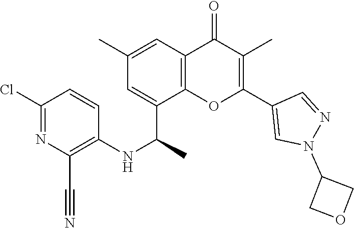 [SMILES: Cc1cc([C@@H](C)Nc2ccc(Cl)nc2C#N)c2oc(-c3cnn(C4COC4)c3)c(C)c(=O)c2c1] | 476 [M + H]⁺ |
| 235C | tert-Butyl 6-chloro-3-[[(1R)- 1-[2-(6,7-dihydro-4H- pyrazolo[5,1-c][1,4]oxazin- 3-yl)-3,6-dimethyl-4-oxo- chromen-8- yl]ethyl]amino]pyridine-2- carboxylate | 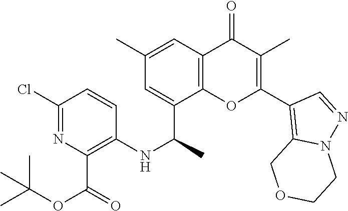 [SMILES: Cc1cc([C@@H](C)Nc2ccc(Cl)nc2C(=O)OC(C)(C)C)c2oc(-c3cnn4c3COCC4)c(C)c(=O)c2c1] | 495 [MH − tBu]⁺ |
| 236C | 8-[(1R)-1-[[2-[(3S,4S)-3,4- Bis[[tert- butyl(dimethyl)silyl]oxy] pyrrolidin-1-yl]-6-chloro-3- pyridyl]amino]ethyl]-3,6- dimethyl-2-(3- pyridyl)chromen-4-one | 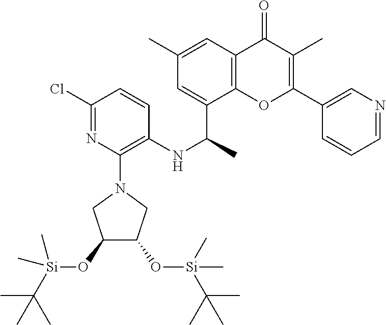 [SMILES: Cc1cc([C@@H](C)Nc2ccc(Cl)nc2N2C[C@H](O[Si](C)(C)C(C)(C)C)[C@@H](O[Si](C)(C)C(C)(C)C)C2)c2oc(-c3cccnc3)c(C)c(=O)c2c1] | 735 [M + H]⁺ |
| 237C | 6-Chloro-3-[[(1R)-1-[3,6- dimethyl-2-[1-[(3- methyloxetan-3- yl)methyl]pyrazol-4-yl]-4- oxo-chromen-8- yl]ethyl]amino]pyridine-2- carbonitrile | 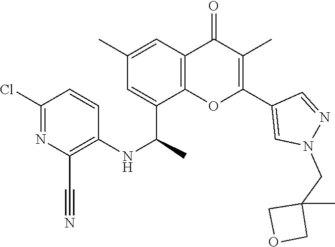 [SMILES: Cc1cc([C@@H](C)Nc2ccc(Cl)nc2C#N)c2oc(-c3cnn(CC4(C)COC4)c3)c(C)c(=O)c2c1] | 504 [M + H]⁺ |
| 238C | tert-Butyl 3-[4-[8-[(1R)-1- [(6-chloro-2-cyano-3- pyridyl)amino]ethyl]-3,6- dimethyl-4-oxo-chromen-2- yl]pyrazol-1-yl]azetidine-1- carboxylate | 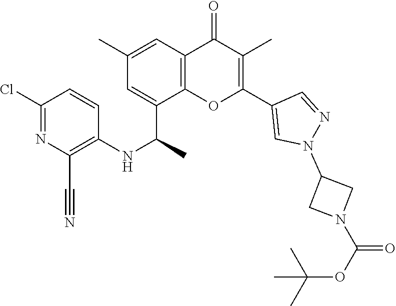 [SMILES: Cc1cc([C@@H](C)Nc2ccc(Cl)nc2C#N)c2oc(-c3cnn(C4CN(C(=O)OC(C)(C)C)C4)c3)c(C)c(=O)c2c1] | 575 [M + H]⁺ |

¹Xantphos Pd G4 used instead of Pd₂(dba)₃ and Xantphos.
²RuPhos Pd G4 used instead of Pd₂(dba)₃ and Xantphos.

### Intermediate 195A: tert-Butyl 6-chloro-3-[[(1R)-1-[3,6-dimethyl-2-(2-methylimidazo[1,2-a]pyridine-6-yl)-4-oxo-chromen-8-yl]ethyl]amino]pyridine-2-carboxylate

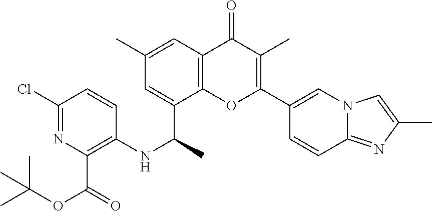 [SMILES: Cc1cc([C@@H](C)Nc2ccc(Cl)nc2C(=O)OC(C)(C)C)c2oc(-c3ccc4nc(C)cn4c3)c(C)c(=O)c2c1]

tert-Butyl 6-chloro-3-[[(1R)-1-(2-ethylsulfanyl-3,6-dimethyl-4-oxo-chromen-8-yl)ethyl]amino]pyridine-2-carboxylate (0.30 g, 0.61 mmol), 2-methyl-6-(4,4,5,5-tetramethyl-1,3,2-dioxaborolan-2-yl)imidazo[1,2-a]pyridine (0.40 g, 1.53 mmol), tetrakis(triphenylphosphine)palladium(0) (0.35 g, 0.31 mmol), copper(I) thiophene-2-carboxylate (0.29 g, 1.53 mmol), and cesium carbonate (0.50 g, 1.53 mmol) were suspended in 6 mL of THF in a tube which was degassed for 10 min, capped, and stirred at 70° C. for 2 h. The reaction was allowed to cool to rt, diluted with 10 mL of DCM, filtered through celite, and concentrated. The residue was purified by silica gel chromatography eluted with 0% to 100% DCM in heptane to give the title compound (0.18 g, 53%). MS ES+ m/z 559 [M+H]⁺.

The following compounds in Table 29 were made in a similar way as described for tert-butyl 6-chloro-3-[[(1R)-1-[3,6-dimethyl-2-(2-methylimidazo[1,2-a]pyridine-6-yl)-4-oxo-chromen-8-yl]ethyl]amino]pyridine-2-carboxylate. Various methods were used to purify the compounds, which would be apparent to one skilled in the art.

TABLE 29

| | | | MS ES+ |
|---|---|---|---|
| Interm # | Chemical Name | Structure | m/z |
| 195A¹ | tert-Butyl 2-[[(1R)-1- [3,6-dimethyl-2-(2- methylindazol-5-yl)-4- oxo-chromen-8- yl]ethyl]amino]benzoate | 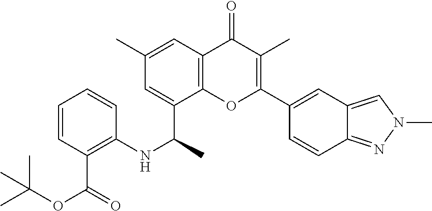 [SMILES: Cc1cc([C@@H](C)Nc2ccccc2C(=O)OC(C)(C)C)c2oc(-c3ccc4nn(C)cc4c3)c(C)c(=O)c2c1] | 524 [M + H]⁺ |
| 196A | 6-Chloro-3-[[(1R)-1- [2-(3-cyano-2-fluoro- phenyl)-3,6-dimethyl- 4-oxo-chromen-8- yl]ethyl]amino]pyridine- 2-carboxylic acid | 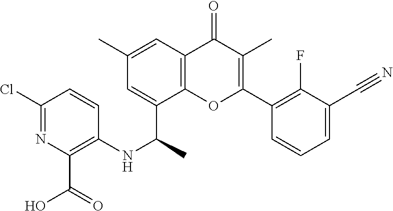 [SMILES: Cc1cc([C@@H](C)Nc2ccc(Cl)nc2C(=O)O)c2oc(-c3cccc(C#N)c3F)c(C)c(=O)c2c1] | 492 [M + H]⁺ |
| 197A | tert-Butyl 6-chloro-3- [[(1R)-1-[2-(1- isopropylpyrazol-4-yl)- 3,6-dimethyl-4-oxo- chromen-8- yl]ethyl]amino]pyridine- 2-carboxylate | 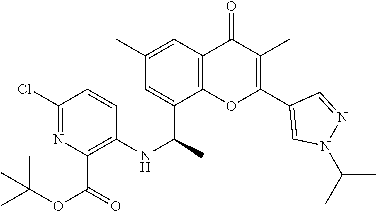 [SMILES: Cc1cc([C@@H](C)Nc2ccc(Cl)nc2C(=O)OC(C)(C)C)c2oc(-c3cnn(C(C)C)c3)c(C)c(=O)c2c1] | |
| 198A² | tert-Butyl 6-chloro-3- [[(1R)-1-(6-methyl-4- oxo-2-phenyl-chromen- 8- yl)ethyl]amino]pyridine- 2-carboxylate | 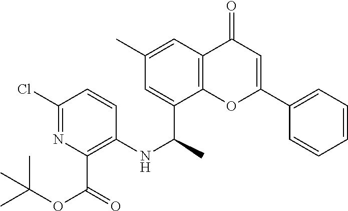 [SMILES: Cc1cc([C@@H](C)Nc2ccc(Cl)nc2C(=O)OC(C)(C)C)c2oc(-c3ccccc3)cc(=O)c2c1] | 491 [M + H]⁺ |
| 199A² | tert-Butyl 6-chloro-3- [[(1R)-1-[6-methyl-2- (2-methylindazol-5-yl)- 4-oxo-chromen-8- yl]ethyl]amino]pyridine- 2-carboxylate | 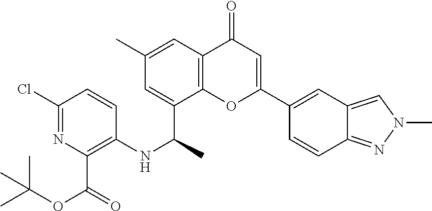 [SMILES: Cc1cc([C@@H](C)Nc2ccc(Cl)nc2C(=O)OC(C)(C)C)c2oc(-c3ccc4nn(C)cc4c3)cc(=O)c2c1] | 545 [M + H]⁺ |
| 200A² | tert-Butyl 6-chloro-3- [[(1R)-1-[3,6-dimethyl- 2-(1-methylpyrazol-3- yl)-4-oxo-chromen-8- yl]ethyl]amino]pyridine- 2-carboxylate | 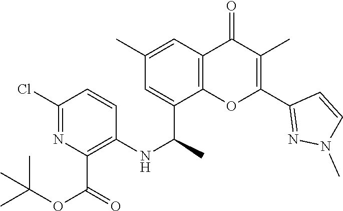 [SMILES: Cc1cc([C@@H](C)Nc2ccc(Cl)nc2C(=O)OC(C)(C)C)c2oc(-c3ccn(C)n3)c(C)c(=O)c2c1] | 453 [M + 2H − tBu]⁺ |
| 201A² | tert-Butyl 6-chloro-3- [[(1R)-1-[6-methyl-2- (1-methylpyrazol-4-yl)- 4-oxo-chromen-8- yl]ethyl]amino]pyridine- 2-carboxylate | 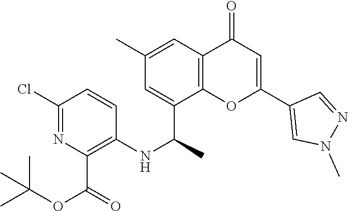 [SMILES: Cc1cc([C@@H](C)Nc2ccc(Cl)nc2C(=O)OC(C)(C)C)c2oc(-c3cnn(C)c3)cc(=O)c2c1] | 495 [M + H]⁺ |
| 202A² | 6-Chloro-3-[[(1R)-1- [3,6-dimethyl-2-(1- methyltriazol-4-yl)-4- oxo-chromen-8- yl]ethyl]amino]pyridine- 2-carboxylic acid | 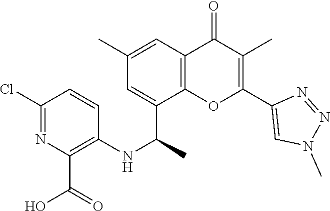 [SMILES: Cc1cc([C@@H](C)Nc2ccc(Cl)nc2C(=O)O)c2oc(-c3cn(C)nn3)c(C)c(=O)c2c1] | 454 [M + H]⁺ |
| 203A² | tert-Butyl 6-chloro-3- [[(1R)-1-[2-(3-cyano-5- fluoro-phenyl)-3,6- dimethyl-4-oxo- chromen-8- yl]ethyl]amino]pyridine- 2-carboxylate | 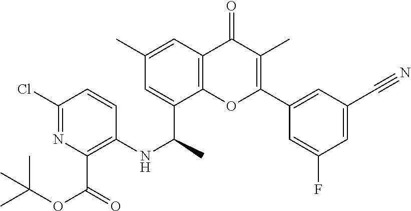 [SMILES: Cc1cc([C@@H](C)Nc2ccc(Cl)nc2C(=O)OC(C)(C)C)c2oc(-c3cc(F)cc(C#N)c3)c(C)c(=O)c2c1] | 492 [M + 2H − tBu]⁺ |
| 204A² | tert-Butyl 6-chloro-3- [[(1R)-1-[2-(5-cyano-3- pyridyl)-3,6-dimethyl- 4-oxo-chromen-8- yl]ethyl]amino]pyridine- 2-carboxylate | 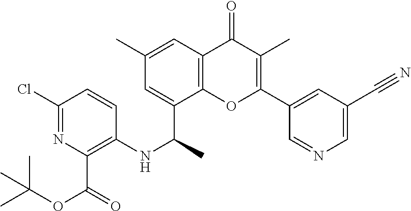 [SMILES: Cc1cc([C@@H](C)Nc2ccc(Cl)nc2C(=O)OC(C)(C)C)c2oc(-c3cncc(C#N)c3)c(C)c(=O)c2c1] | 475 [M + 2H − tBu]⁺ |
| 205A² | tert-Butyl 6-chloro-3- [[(1R)-1-[3,6-dimethyl- 4-oxo-2-(6-oxo-1H- pyridin-2-yl) chromen-8- yl]ethyl]amino]pyridine- 2-carboxylate | 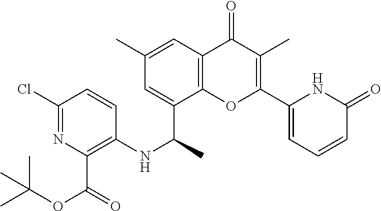 [SMILES: Cc1cc([C@@H](C)Nc2ccc(Cl)nc2C(=O)OC(C)(C)C)c2oc(-c3cccc(=O)[nH]3)c(C)c(=O)c2c1] | 466 [M + 2H − tBu]⁺ |
| 239C² | tert-Butyl 6-chloro-3- [[(1R)-1-[2-(3-cyano-2- fluoro-phenyl)-3,6- dimethyl-4-oxo- chromen-8- yl]ethyl]amino]pyridine- 2-carboxylate | 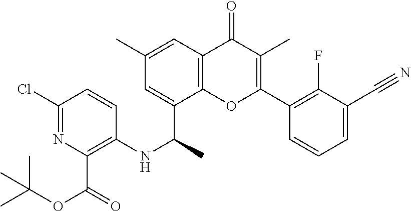 [SMILES: Cc1cc([C@@H](C)Nc2ccc(Cl)nc2C(=O)OC(C)(C)C)c2oc(-c3cccc(C#N)c3F)c(C)c(=O)c2c1] | 548 [M + H]⁺ |
| 240C² | tert-Butyl 6-chloro-3- [[(1R)-1-[3,6-dimethyl- 4-oxo-2-(1H-pyrazol-4- yl)chromen-8- yl]ethyl]amino]pyridine- 2-carboxylate | 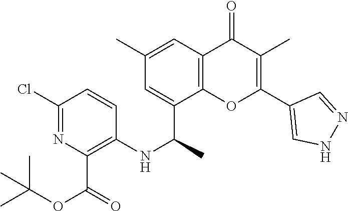 [SMILES: Cc1cc([C@@H](C)Nc2ccc(Cl)nc2C(=O)OC(C)(C)C)c2oc(-c3cn[nH]c3)c(C)c(=O)c2c1] | 439 [MH − ^{t}Bu]⁺ |
| 241C² | tert-Butyl 6-chloro-3- [[(1R)-1-[3,6-dimethyl- 2-(1-meethylindazol-3- yl)-4-oxo-chromen-8- yl]ethyl]amino]pyridine- 2-carboxylate | 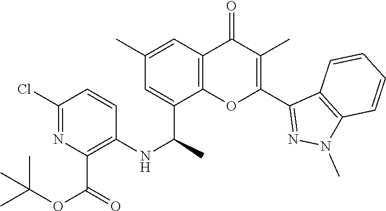 [SMILES: Cc1cc([C@@H](C)Nc2ccc(Cl)nc2C(=O)OC(C)(C)C)c2oc(-c3nn(C)c4ccccc34)c(C)c(=O)c2c1] | 503 [MH − ^{t}Bu]⁺ |
| 242C² | 6-Chloro-3-[[(1R)-1- [3,6-dimethyl-2-(1- methylindazol-3-yl)-4- oxo-chromen-8- yl]ethyl]amino]pyridine- 2-carbonitrile | 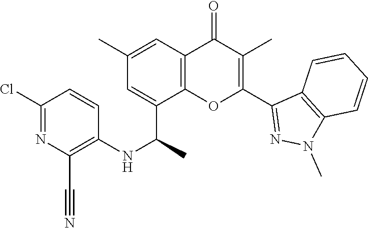 [SMILES: Cc1cc([C@@H](C)Nc2ccc(Cl)nc2C#N)c2oc(-c3nn(C)c4ccccc34)c(C)c(=O)c2c1] | 484 [M + H]⁺ |
| 243C² | tert-Butyl 6-chloro-3- [[(1R)-1-[2-(2-cyano-5- fluoro-phenyl)-3,6- dimethyl-4-oxo- chromen-8- yl]ethyl]amino]pyridine- 2-carboxylate | 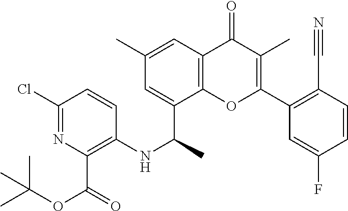 [SMILES: Cc1cc([C@@H](C)Nc2ccc(Cl)nc2C(=O)OC(C)(C)C)c2oc(-c3cc(F)ccc3C#N)c(C)c(=O)c2c1] | 492 [MH − ^{t}Bu]⁺ |
| 244C² | tert-Butyl 6-chloro-3- [[(1R)-1-[2-(2-cyano-3- fluoro-phenyl)-3,6- dimethyl-4-oxo- chromen-8- yl]ethyl]amino]pyridine- 2-carboxylate | 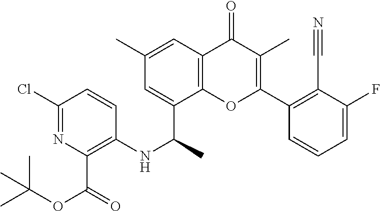 [SMILES: Cc1cc([C@@H](C)Nc2ccc(Cl)nc2C(=O)OC(C)(C)C)c2oc(-c3cccc(F)c3C#N)c(C)c(=O)c2c1] | 492 [MH − ^{t}Bu]⁺ |
| 245C² | tert-Butyl 6-chloro-3- [[(1R)-1-[2-(2- cyanophenyl)-3,6- dimethyl-4-oxo- chromen-8- yl]ethyl]amino]pyridine- 2-carboxylate | 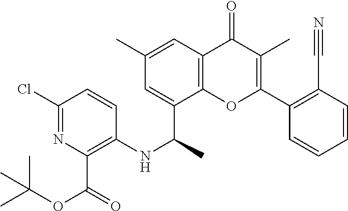 [SMILES: Cc1cc([C@@H](C)Nc2ccc(Cl)nc2C(=O)OC(C)(C)C)c2oc(-c3ccccc3C#N)c(C)c(=O)c2c1] | 474 [MH − ^{t}Bu]⁺ |
| 246C² | tert-Butyl 6-chloro-3- [[(1R)-1-[2-(1,5- dimethylpyrazol-4-yl)- 3,6-dimethyl-4-oxo- chromen-8- yl]ethyl]amino]pyridine- 2-carboxylate | 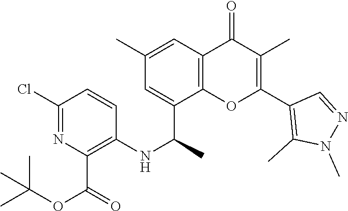 [SMILES: Cc1cc([C@@H](C)Nc2ccc(Cl)nc2C(=O)OC(C)(C)C)c2oc(-c3cnn(C)c3C)c(C)c(=O)c2c1] | 467 [MH − ^{t}Bu]⁺ |
| 247C² | tert-Butyl 6-chloro-3- [[(1R)-1-[2-(1,3- dimethylpyrazol-4-yl)- 3,6-dimethyl-4-oxo- chromen-8- yl]ethyl]amino]pyridine- 2-carboxylate | 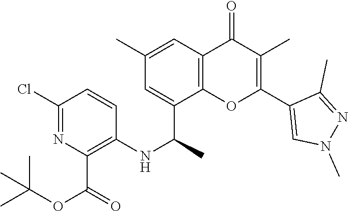 [SMILES: Cc1cc([C@@H](C)Nc2ccc(Cl)nc2C(=O)OC(C)(C)C)c2oc(-c3cn(C)nc3C)c(C)c(=O)c2c1] | 467 [MH − ^{t}Bu]⁺ |
| 248C² | tert-Butyl 6-chloro-3- [[(1R)-1-[2-(5-cyano-2- fluoro-phenyl)-3,6- dimethyl-4-oxo- chromen-8- yl]ethyl]amino]pyridine- 2-carboxylate | 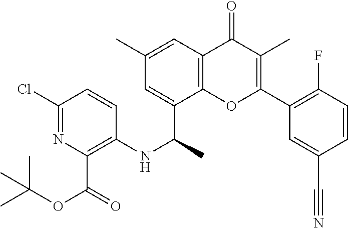 [SMILES: Cc1cc([C@@H](C)Nc2ccc(Cl)nc2C(=O)OC(C)(C)C)c2oc(-c3cc(C#N)ccc3F)c(C)c(=O)c2c1] | 492 [MH − ^{t}Bu]⁺ |
| 249C² | 8-[(1R)-1-[(6-Chloro- 2-methyl-3- pyridyl)amino]ethyl]-6- methyl-2-(3- pyridyl)chromen-4-one | 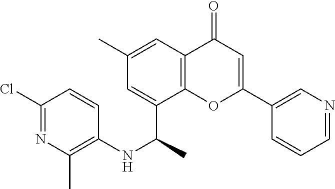 [SMILES: Cc1cc([C@@H](C)Nc2ccc(Cl)nc2C)c2oc(-c3cccnc3)cc(=O)c2c1] | 406 [M + H]⁺ |
| 250C¹ | 6-Chloro-3-[[(1R)-1- [3-isoxazol-4-yl-6- methyl-4-oxo-2-(3- pyridyl)chromen-8- yl]ethyl]amino]pyridine- 2-carbonitrile | 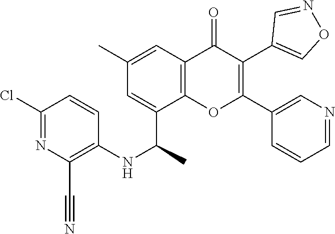 [SMILES: Cc1cc([C@@H](C)Nc2ccc(Cl)nc2C#N)c2oc(-c3cccnc3)c(-c3cnoc3)c(=O)c2c1] | 484 [M + H]⁺ |
| 251C¹ | tert-Butyl 3-[[(1R)-1- [2-(1,3-benzoxazol-4- yl)-3,6-dimethyl-4-oxo- chromen-8- yl]ethyl]amino]-6- chloro-pyridine-2- carboxylate | 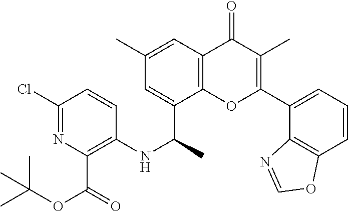 [SMILES: Cc1cc([C@@H](C)Nc2ccc(Cl)nc2C(=O)OC(C)(C)C)c2oc(-c3cccc4ocnc34)c(C)c(=O)c2c1] | 546 [M + H]⁺ |
| 252C | N-tert-Butyl-6-chloro- 3-[[(1R)-1-[2-[1- (difluoromethyl) pyrazol-4-yl]-3,6- dimethyl-4-oxo- chromen-8- yl]ethyl]amino] pyridine-2-sulfonamide | 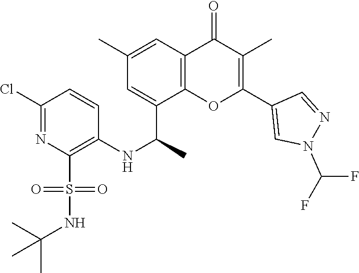 [SMILES: Cc1cc([C@@H](C)Nc2ccc(Cl)nc2S(=O)(=O)NC(C)(C)C)c2oc(-c3cnn(C(F)F)c3)c(C)c(=O)c2c1] | 580 [M + H]⁺ |
| 253C² | 6-Chloro-3-[[(1R)-1- [2-[1-[(2,2-dimethyl- 1,3-dioxolan-4- yl)methyl]pyrazol-4- yl]-3,6-dimethyl-4-oxo- chromen-8- yl]ethyl]amino]pyridine- 2-carbonitrile | 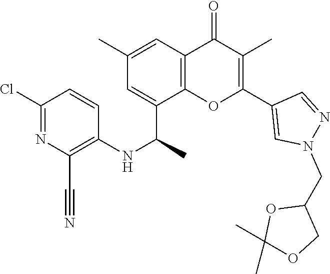 [SMILES: Cc1cc([C@@H](C)Nc2ccc(Cl)nc2C#N)c2oc(-c3cnn(CC4COC(C)(C)O4)c3)c(C)c(=O)c2c1] | 534 [M + H]⁺ |
| 254C² | tert-Butyl 6-chloro-3- [1-[2-(3,4- difluorophenyl)-3- methyl-4-oxo-6- (trifluoromethyl) chromen-8- yl]ethylamino]pyridine- 2-carboxylate, Isomer 2 | 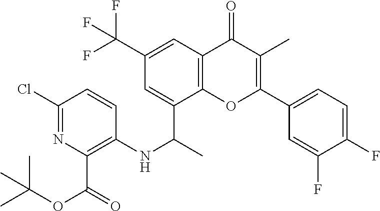 [SMILES: Cc1c(-c2ccc(F)c(F)c2)oc2c(C(C)Nc3ccc(Cl)nc3C(=O)OC(C)(C)C)cc(C(F)(F)F)cc2c1=O] | 539 [MH − ^{t}Bu]⁺ |
| 255C² | 6-Chloro-3-[1-[2-(2,4- difluorophenyl)-3- methyl-4-oxo-6- (trifluoromethyl) chromen-8- yl]ethylamino]pyridine- 2-carboxylic acid, Isomer 2 | 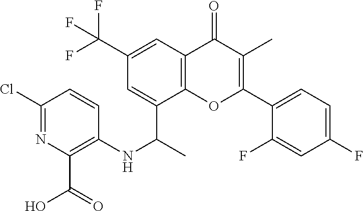 [SMILES: Cc1c(-c2ccc(F)cc2F)oc2c(C(C)Nc3ccc(Cl)nc3C(=O)O)cc(C(F)(F)F)cc2c1=O] | 539 [M + H]⁺ |
| 382C² | N-tert-Butyl-6-chloro- 3-[[(1R)-1-[3,6- dimethyl-4-oxo-2-(2- oxo-1H-pyridin-4- yl)chromen-8- yl]ethyl]amino]pyridine- 2-sulfonamide | 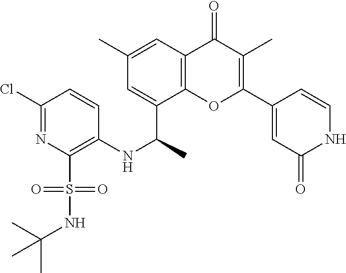 [SMILES: Cc1cc([C@@H](C)Nc2ccc(Cl)nc2S(=O)(=O)NC(C)(C)C)c2oc(-c3cc[nH]c(=O)c3)c(C)c(=O)c2c1] | 557 [M + H]⁺ |

¹No cesium carbonate used in this reaction.
²No cesium carbonate used in this reaction and EtOH used as the solvent.

### Intermediate 256C: 8-[(1R)-1-[[2-(2-Fluorophenyl)-3-pyridyl]amino]ethyl]-6-methyl-2-(3-pyridyl)chromen-4-one

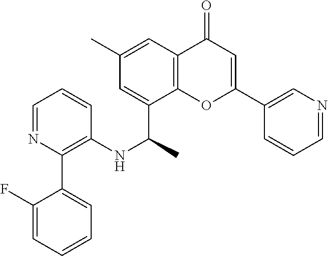 [SMILES: Cc1cc([C@@H](C)Nc2cccnc2-c2ccccc2F)c2oc(-c3cccnc3)cc(=O)c2c1]

Combined 8-[(1R)-1-[(2-chloro-3-pyridyl)amino]ethyl]-6-methyl-2-(3-pyridyl)chromen-4-one (2.46 g, 6.28 mmol), (2-fluorophenyl)boronic acid (0.88 g, 6.28 mmol), potassium carbonate (1.74 g, 12.6 mmol), and tetrakis(triphenylphosphine)palladium(0) (0.36 g, 0.31 mmol) in 1,4-dioxane (20.9 mL) and water (5.46 mL), sparged with argon for 5 min, and stirred at 100° C. for 16 h. The dry reaction was resuspended in DCM and purified by silica gel chromatography eluted with 0% to 100% EtOAc in heptane to give the title compound (0.77 g, 27%) as a yellow foam. ES/MS m/z 452 (M+H).

### Intermediate 257C: tert-Butyl 6-chloro-3-[[(1R)-1-[2-(2,6-difluorophenyl)-3,6-dimethyl-4-oxo-chromen-8-yl]ethyl]amino]pyridine-2-carboxylate

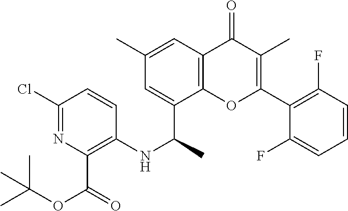 [SMILES: Cc1cc([C@@H](C)Nc2ccc(Cl)nc2C(=O)OC(C)(C)C)c2oc(-c3c(F)cccc3F)c(C)c(=O)c2c1]

Combined tert-butyl 6-chloro-3-[[(1R)-1-(2-ethylsulfanyl-3,6-dimethyl-4-oxo-chromen-8-yl)ethyl]amino]pyridine-2-carboxylate (0.50 g, 1.02 mmol), zinc(II) 2,6-difluorobenzen-1-ide bromide (0.79 g, 0.5 M, 3.07 mmol), methanesulfonato{[4-N,N-dimethylamino)phenyl]di-t-butylphosphino}(2′-amino-1,1′-biphenyl-2-yl)palladium(II) (0.20 g, 0.31 mmol), and copper(I) thiophene-2-carboxylate (0.29 g, 1.53 mmol) in THF (3 mL) and degassed for 5 min. The reaction was stirred at 75° C. for 6 h. The reaction was filtered through diatomaceous earth and the solids washed with 20 mL of DCM. The filtrate was concentrated under reduced pressure to give the title compound (0.90 g, 160%) which was used without purification. ES/MS m/z 485 (MH−^{t}Bu).

### Intermediate 25A: 3-[[(1R)-1-(3,6-Dimethyl-4-oxo-2-phenyl-chromen-8-yl)ethyl]amino]pyridine-2-carboxylic acid

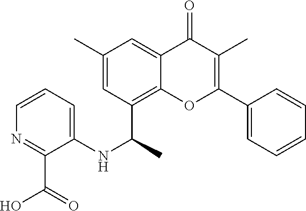 [SMILES: Cc1cc([C@@H](C)Nc2cccnc2C(=O)O)c2oc(-c3ccccc3)c(C)c(=O)c2c1]

Dissolved tert-butyl 3-[[(1R)-1-(3,6-dimethyl-4-oxo-2-phenyl-chromen-8-yl)ethyl]amino]pyridine-2-carboxylate (7.1 g, 15 mmol) in DCM (1 mL) and TFA (1 mL). Sealed and heated the reaction at 50° C. for 16 h. Concentrated and purified by reverse phase chromatography eluted with a gradient of 0% to 100% ACN in aqueous NH₄HCO₃ (10 mM, plus 5% MeOH) to give crude product. Recrystallization from MeOH (50 mL) and MTBE (50 mL) gave the title compound (3.4 g, 54%). MS ES+ m/z 415 [M+H]⁺.

The following compounds in Table 30 were made in a similar way as described for 3-[[(1R)-1-(3,6-dimethyl-4-oxo-2-phenyl-chromen-8-yl)ethyl]amino]pyridine-2-carboxylic acid. Various methods were used to purify the compounds, which would be apparent to one skilled in the art.

TABLE 30

| | | | MS ES+ |
|---|---|---|---|
| Interm # | Chemical Name | Structure | m/z |
| 206A | 6-Chloro-3-[(1R)-1-(3,6- dimethyl-4-oxo-2-phenyl- chromen-8-yl)ethyl]amino]- pyridine-2-carboxylic acid | 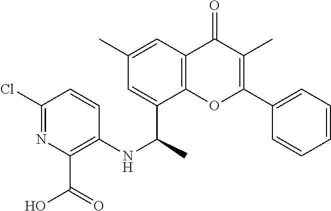 [SMILES: Cc1cc([C@@H](C)Nc2ccc(Cl)nc2C(=O)O)c2oc(-c3ccccc3)c(C)c(=O)c2c1] | 449 [M + H]⁺ |
| 207A | 6-Chloro-3-[(1R)-1-[2- (3,4-difluorophenyl)-3,6- dimethyl-4-oxo-chromen- 8-yl]ethyl]amino]pyri- dine-2-carboxylic acid | 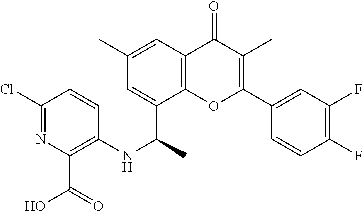 [SMILES: Cc1cc([C@@H](C)Nc2ccc(Cl)nc2C(=O)O)c2oc(-c3ccc(F)c(F)c3)c(C)c(=O)c2c1] | 485 [M + H]⁺ |
| 208A | 3-[[(1R)-1-[3,6-Dimethyl- 2-(2-methylindazol-5-yl)- 4-oxo-chromen-8-yl]- ethyl]amino]-6-methyl- pyridine-2-carboxylic acid | 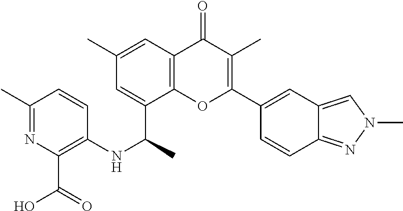 | 483 [M + H]⁺ |
| 209A | 6-Chloro-3-[(1R)-1-[3,6- dimethyl-2-(1-methyl- pyrazol-4-yl)-4-oxo- chromen-8-yl]ethyl]- amino]pyridine-2- carboxylic acid | 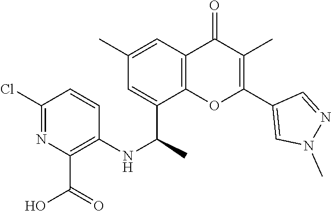 [SMILES: Cc1cc([C@@H](C)Nc2ccc(Cl)nc2C(=O)O)c2oc(-c3cnn(C)c3)c(C)c(=O)c2c1] | 453 [M + H]⁺ |
| 210A | 6-Chloro-3-[[(1R)-1-[3,6- dimethyl-2-(2-methyl- indazol-5-yl)-4-oxo- chromen-8-yl]ethyl]- amino]pyridine-2- carboxylic acid | 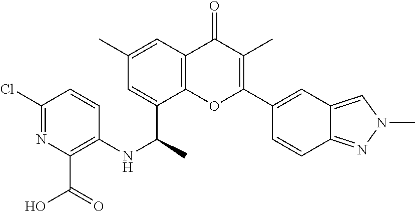 [SMILES: Cc1cc([C@@H](C)Nc2ccc(Cl)nc2C(=O)O)c2oc(-c3ccc4nn(C)cc4c3)c(C)c(=O)c2c1] | 503 [M + H]⁺ |
| 211A | 6-Chloro-3-[(1R)-1-(3,6- dimethyl-4-oxo-2-phenyl- chromen-8-yl)ethyl]- amino]pyridine-2-carbox- ylic acid | 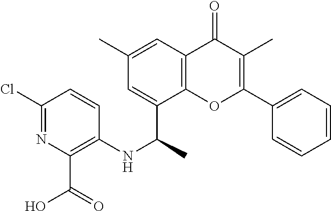 [SMILES: Cc1cc([C@@H](C)Nc2ccc(Cl)nc2C(=O)O)c2oc(-c3ccccc3)c(C)c(=O)c2c1] | 449 [M + H]⁺ |
| 212A | 6-Chloro-3-[[(1R)-1-[2- [1-(difluoromethyl)pyr- azol-4-yl]-3,6-dimethyl-4- oxo-chromen-8-yl]ethyl]- amino]pyridine-2-carbox- ylic acid | 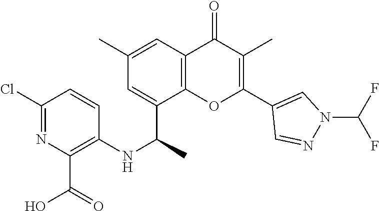 [SMILES: Cc1cc([C@@H](C)Nc2ccc(Cl)nc2C(=O)O)c2oc(-c3cnn(C(F)F)c3)c(C)c(=O)c2c1] | 489 [M + H]⁺ |
| 213A | 6-Chloro-3-[(1R)-1-[3,6- dimethyl-4-oxo-2-(2- pyridyl)chromen-8-yl]- ethyl]amino]pyridine-2- carboxylic acid | 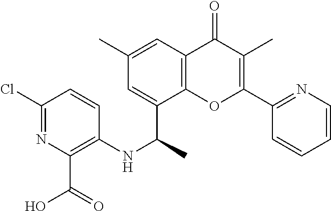 [SMILES: Cc1cc([C@@H](C)Nc2ccc(Cl)nc2C(=O)O)c2oc(-c3ccccn3)c(C)c(=O)c2c1] | 450 [M + H]⁺ |
| 214A | 3-[[(1R)-1-(3,6-Dimethyl- 4-oxo-2-phenyl-chromen- 8-yl)ethyl]amino]pyridine- 2-carboxylic acid | 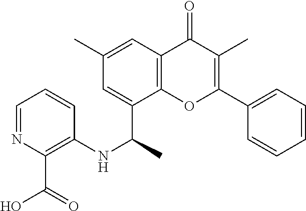 [SMILES: Cc1cc([C@@H](C)Nc2cccnc2C(=O)O)c2oc(-c3ccccc3)c(C)c(=O)c2c1] | 415 [M + H]⁺ |
| 215A | 3-[[(1R)-1-(3,6-Dimethyl- 4-oxo-2-phenyl-chromen- 8-yl)ethyl]amino]-6-meth- yl-pyridine-2-carboxylic acid | 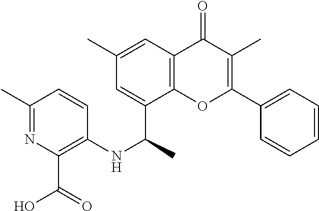 [SMILES: Cc1cc([C@@H](C)Nc2ccc(C)nc2C(=O)O)c2oc(-c3ccccc3)c(C)c(=O)c2c1] | 429 [M + H]⁺ |
| 216A | 3-[[(1R)-1-(3,6-Dimethyl- 4-oxo-2-phenyl-chromen- 8-yl)ethyl]amino]pyri- dine-2-carboxylic acid | 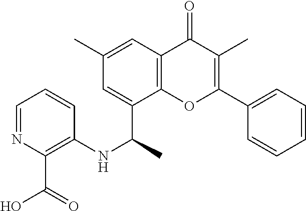 [SMILES: Cc1cc([C@@H](C)Nc2cccnc2C(=O)O)c2oc(-c3ccccc3)c(C)c(=O)c2c1] | 415 [M + H]⁺ |
| 217A | 6-[[(1R)-1-(3,6-Dimethyl- 4-oxo-2-phenyl-chromen- 8-yl)ethyl]amino]-2,3- difluoro-benzoic acid | 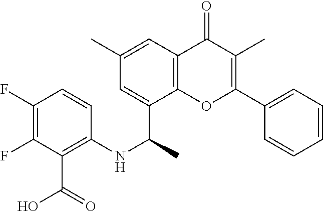 [SMILES: Cc1cc([C@@H](C)Nc2ccc(F)c(F)c2C(=O)O)c2oc(-c3ccccc3)c(C)c(=O)c2c1] | 450 [M + H]⁺ |
| 218A | 6-Chloro-3-[[(1R)-1-[3,6- dimethyl-2-(2-methyl- imidazo[1,2-a]pyridine-6- yl)-4-oxo-chromen-8-yl]- ethyl]amino]pyridine-2- carboxylic acid | 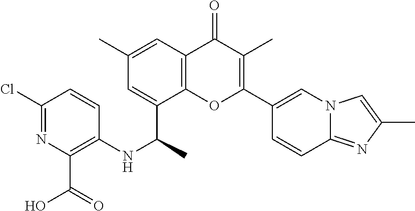 [SMILES: Cc1cc([C@@H](C)Nc2ccc(Cl)nc2C(=O)O)c2oc(-c3ccc4nc(C)cn4c3)c(C)c(=O)c2c1] | 503 [M + H]⁺ |
| 219A | 3-[[(1R)-1-[2-(3,4- Difluorophenyl)- 3,6-dimethyl-4-oxo- chromen-8-yl]ethyl]- amino]-6-methyl- pyridine-2-carbox- ylic acid | 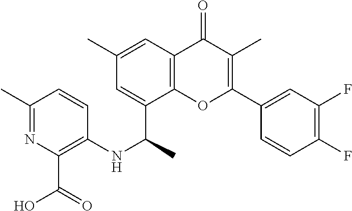 [SMILES: Cc1cc([C@@H](C)Nc2ccc(C)nc2C(=O)O)c2oc(-c3ccc(F)c(F)c3)c(C)c(=O)c2c1] | 465 [M + H]⁺ |
| 220A | 3-[[(1R)-1-[3,6-Dimethyl- 4-oxo-2-(3-pyridyl)- chromen-8-yl]ethyl]- amino]pyridine-2- carboxylic acid | 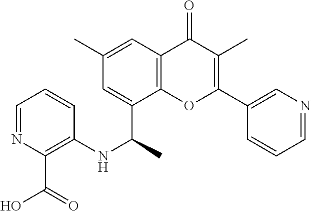 [SMILES: Cc1cc([C@@H](C)Nc2cccnc2C(=O)O)c2oc(-c3cccnc3)c(C)c(=O)c2c1] | 416 [M + H]⁺ |
| 221A | 3-[(1R)-1-[2-[6- (Difluoromethyl)-2- pyridyl]-3,6-dimethyl-4- oxo-chromen-8-yl]ethyl]- amino]pyridine-2-carbox- ylic acid | 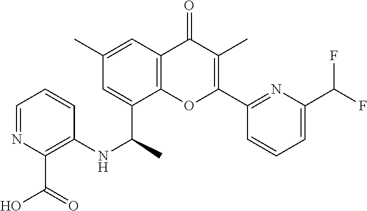 [SMILES: Cc1cc([C@@H](C)Nc2cccnc2C(=O)O)c2oc(-c3cccc(C(F)F)n3)c(C)c(=O)c2c1] | 466 [M + H]⁺ |
| 222A | 2-[[(1R)-1-[3,6-Dimethyl- 4-oxo-2-(3-pyridyl)- chromen-8-yl]ethyl]- amino]benzoic acid | 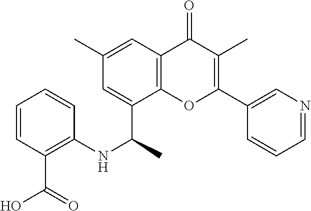 [SMILES: Cc1cc([C@@H](C)Nc2ccccc2C(=O)O)c2oc(-c3cccnc3)c(C)c(=O)c2c1] | 415 [M + H]⁺ |
| 223A | 2-[[(1R)-1-[3,6-Dimethyl- 2-(2-methylindazol-5-yl)- 4-oxo-chromen-8-yl]- ethyl]amino]benzoic acid | 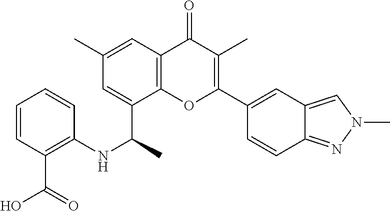 [SMILES: Cc1cc([C@@H](C)Nc2ccccc2C(=O)O)c2oc(-c3ccc4nn(C)cc4c3)c(C)c(=O)c2c1] | 468 [M + H]⁺ |
| 224A | 6-Chloro-3-[(1R)-1-[3,6- dimethyl-4-oxo-2-(3- pyridyl)chromen-8-yl]- ethyl]amino]pyridine-2- carboxylic acid | 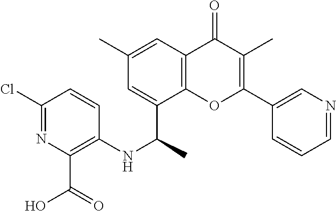 [SMILES: Cc1cc([C@@H](C)Nc2ccc(Cl)nc2C(=O)O)c2oc(-c3cccnc3)c(C)c(=O)c2c1] | 450 [M + H]⁺ |
| 225A | 3-[[(1R)-1-[3,6-Dimethyl- 2-(2-methylindazol-5-yl)- 4-oxo-chromen-8-yl]- ethyl]amino]pyridine-2- carboxylic acid | 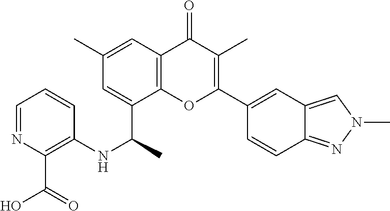 [SMILES: Cc1cc([C@@H](C)Nc2cccnc2C(=O)O)c2oc(-c3ccc4nn(C)cc4c3)c(C)c(=O)c2c1] | 469 [M + H]⁺ |
| 226A | 6-Chloro-3-[[(1R)-1-[2- (1-isopropylpyrazol-4-yl)- 3,6-dimethyl-4-oxo- chromen-8-yl]ethyl]- amino]pyridine-2- carboxylic acid | 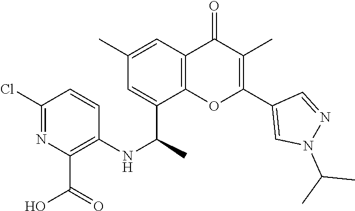 [SMILES: Cc1cc([C@@H](C)Nc2ccc(Cl)nc2C(=O)O)c2oc(-c3cnn(C(C)C)c3)c(C)c(=O)c2c1] | 481 [M + H]⁺ |
| 227A | 6-Chloro-3-[(1R)-1-[6- methyl-2-(2-methyl- indazol-5-yl)-4-oxo- chromen-8-yl]ethyl]- amino]pyridine-2- carboxylic acid | 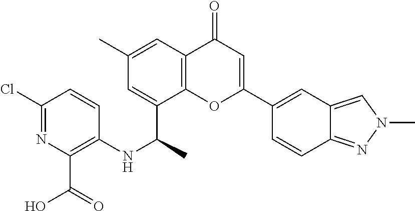 [SMILES: Cc1cc([C@@H](C)Nc2ccc(Cl)nc2C(=O)O)c2oc(-c3ccc4nn(C)cc4c3)cc(=O)c2c1] | 489 [M + H]⁺ |
| 228A | 6-Chloro-3-[[(1R)-1-[3- methyl-2-(1-methyl- pyrazol-4-yl)-4-oxo-6- (trifluoromethyl)chromen- 8-yl]ethyl]amino]pyridine- 2-carboxylic acid | 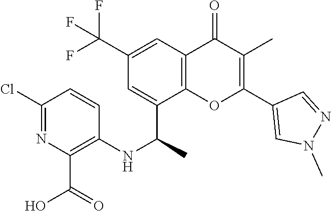 [SMILES: Cc1c(-c2cnn(C)c2)oc2c([C@@H](C)Nc3ccc(Cl)nc3C(=O)O)cc(C(F)(F)F)cc2c1=O] | 507 [M + H]⁺ |
| 229A | 6-Chloro-3-[(1R)-1-[3,6- dimethyl-2-(1-methyl- pyrazol-3-yl)-4-oxo- chromen-8-yl]ethyl]- amino]pyridine-2- carboxylic acid | 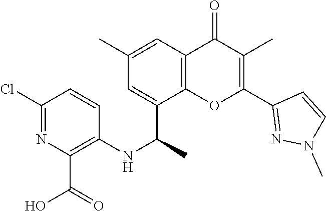 [SMILES: Cc1cc([C@@H](C)Nc2ccc(Cl)nc2C(=O)O)c2oc(-c3ccn(C)n3)c(C)c(=O)c2c1] | 453 [M + H]⁺ |
| 230A | 6-Chloro-3-[(1R)-1-[6- methyl-2-(1-methylpyr- azol-4-yl)-4-oxo-chromen- 8-yl]ethyl]amino]pyridine- 2-carboxylic acid | 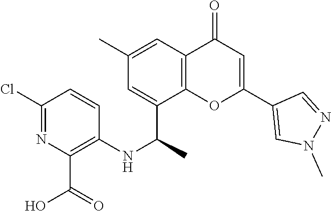 [SMILES: Cc1cc([C@@H](C)Nc2ccc(Cl)nc2C(=O)O)c2oc(-c3cnn(C)c3)cc(=O)c2c1] | 439 [M + H]⁺ |
| 231A | 6-Chloro-3-[[(1R)-1-[2- (6-fluoro-2-pyridyl)-3,6- dimethyl-4-oxo-chromen- 8-yl]ethyl]amino]pyridine- 2-carboxylic acid | 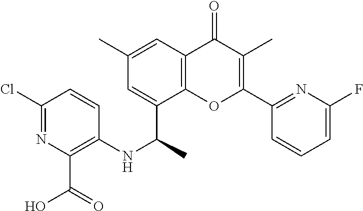 [SMILES: Cc1cc([C@@H](C)Nc2ccc(Cl)nc2C(=O)O)c2oc(-c3cccc(F)n3)c(C)c(=O)c2c1] | 468 [M + H]⁺ |
| 232A | 6-Chloro-3-[(1R)-1-[2- (3-cyano-5-fluorophenyl)- 3,6-dimethyl-4-oxo- chromen-8-yl]ethyl]- amino]pyridine-2- carboxylic acid | 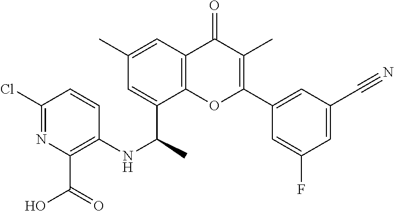 [SMILES: Cc1cc([C@@H](C)Nc2ccc(Cl)nc2C(=O)O)c2oc(-c3cc(F)cc(C#N)c3)c(C)c(=O)c2c1] | 492 [M + H]⁺ |
| 233A | 6-Chloro-3-[(1R)-1-[2- (5-cyano-3-pyridyl)-3,6- dimethyl-4-oxo-chromen- 8-yl]ethyl]amino]pyridine- 2-carboxylic acid | 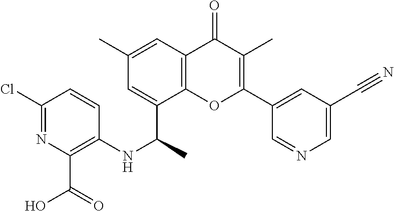 [SMILES: Cc1cc([C@@H](C)Nc2ccc(Cl)nc2C(=O)O)c2oc(-c3cncc(C#N)c3)c(C)c(=O)c2c1] | 475 [M + H]⁺ |
| 234A | 6-Chloro-3-[[(1R)-1-[3,6- dimethyl-4-oxo-2-(6-oxo- 1H-pyridin-2-yl)chromen- 8-yl]ethyl]amino]pyridine- 2-carboxylic acid | 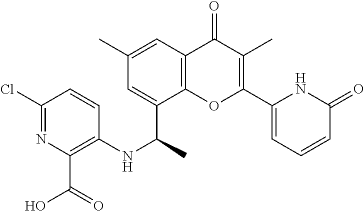 [SMILES: Cc1cc([C@@H](C)Nc2ccc(Cl)nc2C(=O)O)c2oc(-c3cccc(=O)[nH]3)c(C)c(=O)c2c1] | 466 [M + H]⁺ |
| 258C | 6-[[(1R)-1-[3,6-Dimethyl- 4-oxo-2-(3-pyridyl)- chromen-8-yl]ethyl]- amino]-2,3-difluoro- benzoic acid | 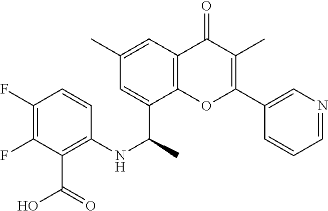 [SMILES: Cc1cc([C@@H](C)Nc2ccc(F)c(F)c2C(=O)O)c2oc(-c3cccnc3)c(C)c(=O)c2c1] | 451 [M + H]⁺ |
| 259C | 6-Chloro-3-[[(1R)-1-[3,6- dimethyl-2-[1-(oxetan-3- yl)pyrazol-4-yl]-4-oxo- chromen-8-yl]ethyl]- amino]pyridine-2-carbox- ylic acid | 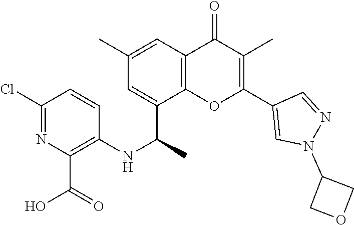 [SMILES: Cc1cc([C@@H](C)Nc2ccc(Cl)nc2C(=O)O)c2oc(-c3cnn(C4COC4)c3)c(C)c(=O)c2c1] | 495 [M + H]⁺ |
| 260C | 6-[[(1R)-1-[3,6-Dimethyl- 2-(1-methylpyrazol-4-yl)- 4-oxo-chromen-8-yl]- ethyl]amino]-2,3- difluoro-benzoic acid [SHH-00001-050] | 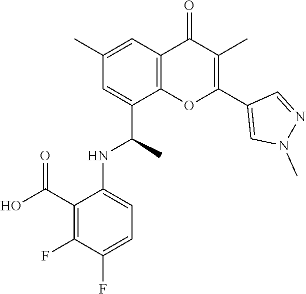 [SMILES: Cc1cc([C@@H](C)Nc2ccc(F)c(F)c2C(=O)O)c2oc(-c3cnn(C)c3)c(C)c(=O)c2c1] | |
| 261C | 6-Chloro-3-[(1R)-1-[2- [6-(1-cyanocyclopropyl)- 3-pyridyl]-3,6-dimethyl- 4-oxo-chromen-8-yl]- ethyl]amino]pyridine- 2-carboxylic acid | 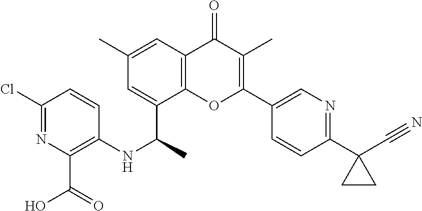 [SMILES: Cc1cc([C@@H](C)Nc2ccc(Cl)nc2C(=O)O)c2oc(-c3ccc(C4(C#N)CC4)nc3)c(C)c(=O)c2c1] | 515 [M + H]⁺₋ |
| 262C | 6-Chloro-3-[[(1R)-1- (2-ethylsulfanyl-3,6- dimethyl-4-oxo-chromen- 8-yl)ethyl]amino]pyridine- 2-carboxylic acid | 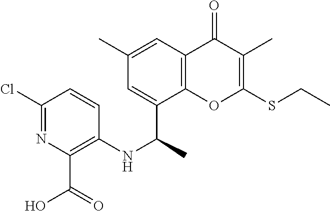 [SMILES: CCSc1oc2c([C@@H](C)Nc3ccc(Cl)nc3C(=O)O)cc(C)cc2c(=O)c1C] | 433 [M + H]⁺ |
| 263C | 3-[[(1R)-1-[2-(1,3- Benzodioxol-5-yl)- 3,6-dimethyl-4-oxo- chromen-8-yl]ethyl]- amino]-6-chloro- pyridine-2-carboxylic acid | 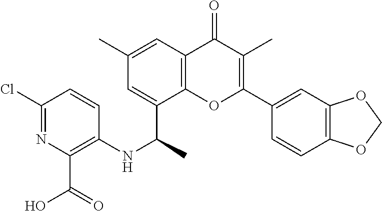 [SMILES: Cc1cc([C@@H](C)Nc2ccc(Cl)nc2C(=O)O)c2oc(-c3ccc4c(c3)OCO4)c(C)c(=O)c2c1] | 493 [M + H]⁺ |
| 264C | 3-[[(1R)-1-[3,6-Dimethyl- 2-(1-methylpyrazol-4-yl)- 4-oxo-chromen-8-yl]- ethyl]amino]-6-fluoro- pyridine-2-carboxylic acid | 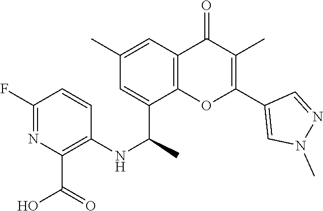 [SMILES: Cc1cc([C@@H](C)Nc2ccc(F)nc2C(=O)O)c2oc(-c3cnn(C)c3)c(C)c(=O)c2c1] | 437 [M + H]⁺ |
| 265C | 6-Chloro-3-[(1R)-1-[2- (3-cyano-2-fluorophen- yl)-3,6-dimethyl-4-oxo- chromen-8-yl]ethyl]- amino]pyridine-2- carboxylic acid | 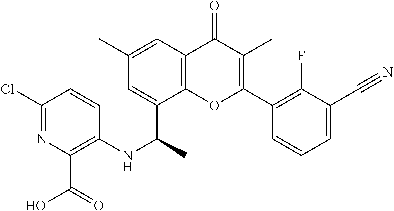 [SMILES: Cc1cc([C@@H](C)Nc2ccc(Cl)nc2C(=O)O)c2oc(-c3cccc(C#N)c3F)c(C)c(=O)c2c1] | 492 [M + H]⁺ |
| 266C | 6-[[(1R)-1-[3,6-Dimethyl- 2-(2-methylindazol-5-yl)- 4-oxo-chromen-8-yl]- ethyl]amino]-2,3- difluoro-benzoic acid | 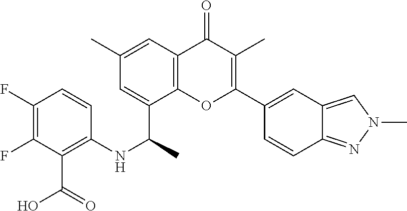 [SMILES: Cc1cc([C@@H](C)Nc2ccc(F)c(F)c2C(=O)O)c2oc(-c3ccc4nn(C)cc4c3)c(C)c(=O)c2c1] | 504 [M + H]⁺ |
| 267C | 6-Chloro-3-[(1R)-1-[3,6- dimethyl-4-oxo-2-(1H- pyrazol-4-yl)chromen-8- yl]ethyl]amino]pyridine- 2-carboxylic acid | 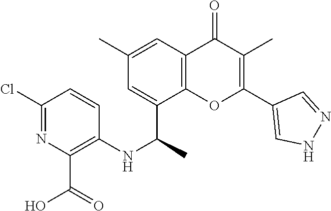 [SMILES: Cc1cc([C@@H](C)Nc2ccc(Cl)nc2C(=O)O)c2oc(-c3cn[nH]c3)c(C)c(=O)c2c1] | 439 [M + H]⁺ |
| 268C | 6-Chloro-3-[[(1R)-1-[3,6- dimethyl-2-(1-methyl- indazol-3-yl)-4-oxo- chromen-8-yl]ethyl]- amino]pyridine-2- carboxylic acid | 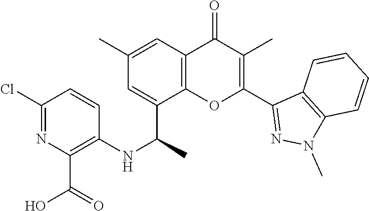 [SMILES: Cc1cc([C@@H](C)Nc2ccc(Cl)nc2C(=O)O)c2oc(-c3nn(C)c4ccccc34)c(C)c(=O)c2c1] | 503 [M + H]⁺ |
| 269C | 6-Chloro-3-[[(1R)-1-[2- (2-cyano-5-fluorophen- yl)-3,6-dimethyl-4-oxo- chromen-8-yl]ethyl]- amino]pyridine-2-carbox- ylic acid | 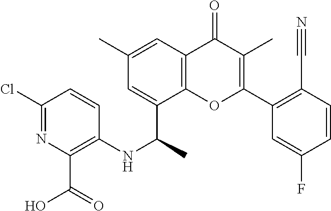 [SMILES: Cc1cc([C@@H](C)Nc2ccc(Cl)nc2C(=O)O)c2oc(-c3cc(F)ccc3C#N)c(C)c(=O)c2c1] | 492 [M + H]⁺ |
| 270C | 6-Chloro-3-[(1R)-1-[2- (2-cyano-3-fluorophen- yl)-3,6-dimethyl-4-oxo- chromen-8-yl]ethyl]- amino]pyridine-2-carbox- ylic acid | 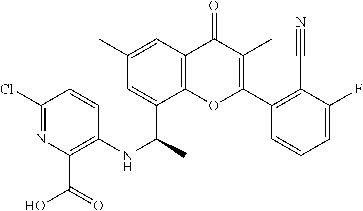 [SMILES: Cc1cc([C@@H](C)Nc2ccc(Cl)nc2C(=O)O)c2oc(-c3cccc(F)c3C#N)c(C)c(=O)c2c1] | 492 [M + H]⁺ |
| 271C | 6-Chloro-3-[[(1R)-1-[2- (2-cyanophenyl)-3,6- dimethyl-4-oxo-chromen- 8-yl]ethyl]amino]pyridine- 2-carboxylic acid | 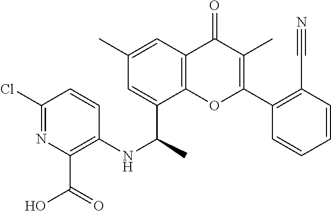 [SMILES: Cc1cc([C@@H](C)Nc2ccc(Cl)nc2C(=O)O)c2oc(-c3ccccc3C#N)c(C)c(=O)c2c1] | 472 [M − H]⁻ |
| 272C | 6-Chloro-3-[(1R)-1-[2- (1,5-dimethylpyrazol-4- yl)-3,6-dimethyl-4-oxo- chromen-8-yl]ethyl]- amino]pyridine-2-carbox- ylic acid | 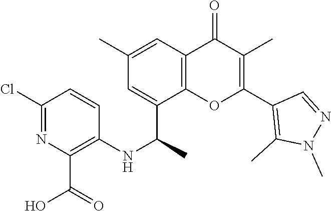 [SMILES: Cc1cc([C@@H](C)Nc2ccc(Cl)nc2C(=O)O)c2oc(-c3cnn(C)c3C)c(C)c(=O)c2c1] | 467 [M + H]⁺ |
| 273C | 6-Chloro-3-[(1R)-1-[2- (1,3-dimethylpyrazol-4- yl)-3,6-dimethyl-4-oxo- chromen-8-yl]ethyl]- amino]pyridine-2-carbox- ylic acid | 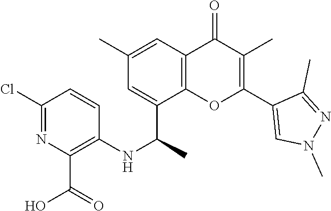 [SMILES: Cc1cc([C@@H](C)Nc2ccc(Cl)nc2C(=O)O)c2oc(-c3cn(C)nc3C)c(C)c(=O)c2c1] | 467 [M + H]⁺ |
| 274C | 6-Chloro-3-[[(1R)-1-[2- (5-cyano-2-fluorophenyl)- 3,6-dimethyl-4-oxo- chromen-8-yl]ethyl]- amino]pyridine-2-carbox- ylic acid | 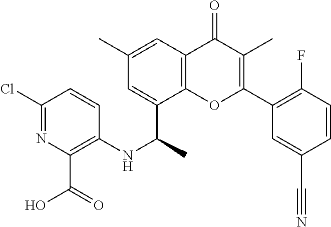 [SMILES: Cc1cc([C@@H](C)Nc2ccc(Cl)nc2C(=O)O)c2oc(-c3cc(C#N)ccc3F)c(C)c(=O)c2c1] | 492 [M + H]⁺ |
| 275C | 6-Chloro-3-[(1R)-1-[3,6- dimethyl-2-(1-methylind- azol-5-yl)-4-oxo-chromen- 8-yl]ethyl]amino]pyridine- 2-carboxylic acid | 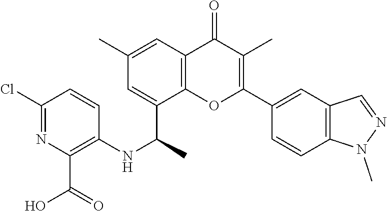 [SMILES: Cc1cc([C@@H](C)Nc2ccc(Cl)nc2C(=O)O)c2oc(-c3ccc4c(cnn4C)c3)c(C)c(=O)c2c1] | 503 [M + H]⁺ |
| 276C | 6-Chloro-3-[[(1R)-1-[2- (2,6-difluorophenyl)-3,6- dimethyl-4-oxo-chromen- 8-yl]ethyl]amino]pyridine- 2-carboxylic acid | 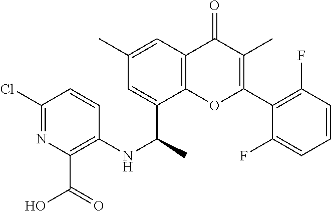 [SMILES: Cc1cc([C@@H](C)Nc2ccc(Cl)nc2C(=O)O)c2oc(-c3c(F)cccc3F)c(C)c(=O)c2c1] | 485 [M + H]⁺ |
| 277C | 6-Chloro-3-[(1R)-1-[2- (2,4-difluorophenyl)-3,6- dimethyl-4-oxo-chromen- 8-yl]ethyl]amino]pyridine- 2-carboxylic acid | 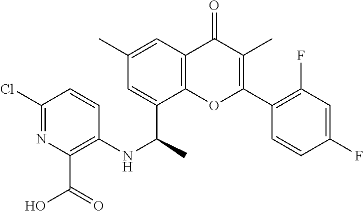 [SMILES: Cc1cc([C@@H](C)Nc2ccc(Cl)nc2C(=O)O)c2oc(-c3ccc(F)cc3F)c(C)c(=O)c2c1] | 485 [M + H]⁺ |
| 278C | 6-Chloro-3-[[(1R)-1-[2- (2,3-difluorophenyl)-3,6- dimethyl-4-oxo-chromen- 8-yl]ethyl]amino]pyridine- 2-carboxylic acid | 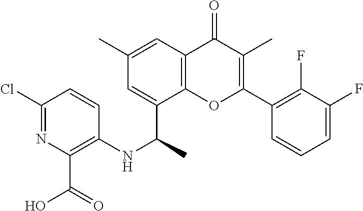 [SMILES: Cc1cc([C@@H](C)Nc2ccc(Cl)nc2C(=O)O)c2oc(-c3cccc(F)c3F)c(C)c(=O)c2c1] | 485 [M + H]⁺ |
| 279C | 6-Chloro-3-[[(1R)-1-[3,6- dimethyl-2-(2-methyl- pyrazolo[3,4-b]pyridin-5- yl)-4-oxo-chromen-8-yl]- ethyl]amino]pyridine-2- carboxylic acid | 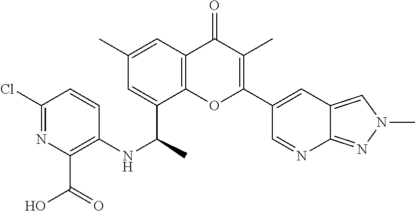 [SMILES: Cc1cc([C@@H](C)Nc2ccc(Cl)nc2C(=O)O)c2oc(-c3cnc4nn(C)cc4c3)c(C)c(=O)c2c1] | 504 [M + H]⁺ |
| 280C | 6-Chloro-3-[[(1R)-1-[3- chloro-6-methyl-2-(1- methylpyrazol-4-yl)-4- oxo-chromen-8-yl]ethyl]- amino]pyridine-2-carbox- ylic acid | 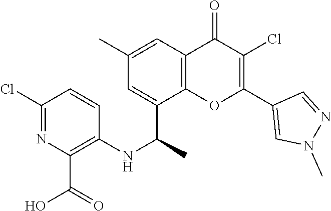 [SMILES: Cc1cc([C@@H](C)Nc2ccc(Cl)nc2C(=O)O)c2oc(-c3cnn(C)c3)c(Cl)c(=O)c2c1] | 471 [M − H]⁻ |
| 281C | 6-Chloro-3-[(1R)-1-[2- (6,7-dihydro-4H- pyrazolo[5,1-c][1,4]- oxazin-3-yl)-3,6- dimethyl-4-oxo- chromen-8-yl]ethyl]- amino]pyridine-2- carboxylic acid | 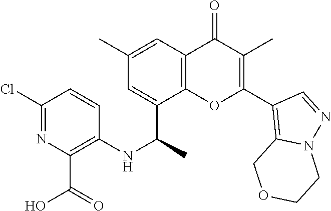 [SMILES: Cc1cc([C@@H](C)Nc2ccc(Cl)nc2C(=O)O)c2oc(-c3cnn4c3COCC4)c(C)c(=O)c2c1] | 495 [M + H]⁺ |
| 282C | 3-[[(1R)-1-[2-(1,3- Benzoxazol-4-yl)-3,6- dimethyl-4-oxo-chromen- 8-yl]ethyl]amino]-6- chloro-pyridine-2- carboxylic acid | 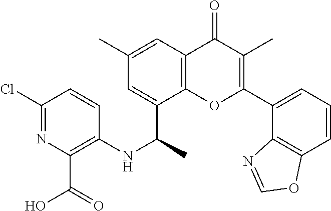 [SMILES: Cc1cc([C@@H](C)Nc2ccc(Cl)nc2C(=O)O)c2oc(-c3cccc4ocnc34)c(C)c(=O)c2c1] | 490 [M + H]⁺ |
| 283C | 6-Chloro-3-[(1R)-1-[3,6- dimethyl-4-oxo-2- (4,5,6,7-tetrahydropyr- azolo[1,5-a]pyrazin-3-yl)- chromen-8-yl]ethyl]- amino]pyridine-2-carbox- ylic acid | 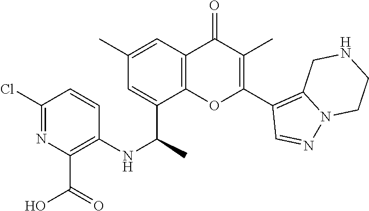 [SMILES: Cc1cc([C@@H](C)Nc2ccc(Cl)nc2C(=O)O)c2oc(-c3cnn4c3CNCC4)c(C)c(=O)c2c1] | 494 [M + H]⁺ |
| 284C | 6-Chloro-3-[(1R)-1-(3,6- dimethyl-4-oxo-2- pyrazolo[1,5-a]pyrimidin- 3-yl-chromen-8-yl)ethyl]- amino]pyridine-2-carbox- ylic acid | 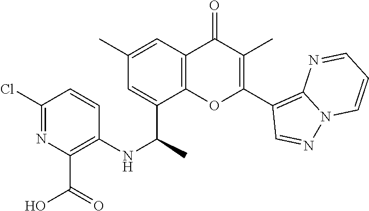 [SMILES: Cc1cc([C@@H](C)Nc2ccc(Cl)nc2C(=O)O)c2oc(-c3cnn4cccnc34)c(C)c(=O)c2c1] | 490 [M + H]⁺ |
| 285C | 6-Chloro-3-[(1R)-1-[3,6- dimethyl-4-oxo-2-[3- (trifluoromethyl)-1- bicyclo[1.1.1]pentanyl]- chromen-8-yl]ethyl]- amino]pyridine-2-carbox- ylic acid | 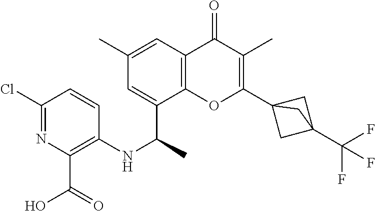 [SMILES: Cc1cc([C@@H](C)Nc2ccc(Cl)nc2C(=O)O)c2oc(C34CC(C(F)(F)F)(C3)C4)c(C)c(=O)c2c1] | 507 [M + H]⁺ |
| 286C | 6-Chloro-3-[1-[6-fluoro- 3-methyl-2-(1-methyl- pyrazol-4-yl)-4-oxo- chromen-8-yl]ethyl- amino]pyridine-2-carbox- ylic acid, Isomer 1 | 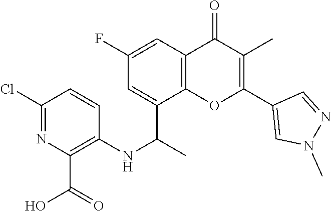 [SMILES: Cc1c(-c2cnn(C)c2)oc2c(C(C)Nc3ccc(Cl)nc3C(=O)O)cc(F)cc2c1=O] | 457 [M + H]⁺ |
| 287C | 6-Chloro-3-[(1R)-1-(2- cyclobutyl-3,6-dimethyl- 4-oxo-chromen-8-yl)- ethyl]amino]pyridine-2- carboxylic acid | 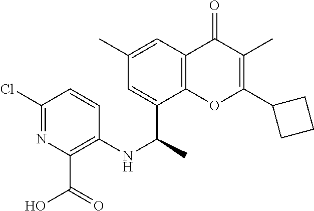 [SMILES: Cc1cc([C@@H](C)Nc2ccc(Cl)nc2C(=O)O)c2oc(C3CCC3)c(C)c(=O)c2c1] | 427 [M + H]⁺ |
| 288C | 6-Chloro-3-[[(1R)-1-[2- (4,4-difluorocyclohexyl)- 3,6-dimethyl-4-oxo- chromen-8-yl]ethyl]- amino]pyridine-2- carboxylic acid | 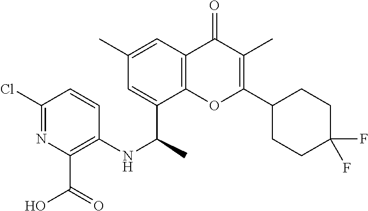 [SMILES: Cc1cc([C@@H](C)Nc2ccc(Cl)nc2C(=O)O)c2oc(C3CCC(F)(F)CC3)c(C)c(=O)c2c1] | 491 [M + H]⁺ |
| 289C | 6-Chloro-3-[(1R)-1-(2- cyclopropyl-3,6-dimethyl- 4-oxo-chromen-8-yl)- ethyl]amino]pyridine-2- carboxylic acid | 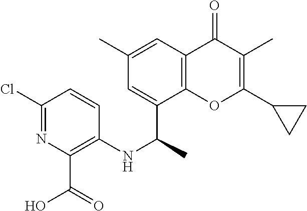 [SMILES: Cc1cc([C@@H](C)Nc2ccc(Cl)nc2C(=O)O)c2oc(C3CC3)c(C)c(=O)c2c1] | 413 [M + H]⁺ |
| 290C | 6-Chloro-3-[1-[2-(3,4- difluorophenyl)-3-methyl- 4-oxo-6-(trifluorometh- yl)chromen-8-yl]ethyl- amino]pyridine-2-carbox- ylic acid, Isomer 2 | 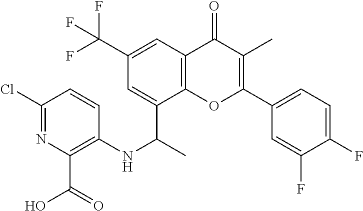 [SMILES: Cc1c(-c2ccc(F)c(F)c2)oc2c(C(C)Nc3ccc(Cl)nc3C(=O)O)cc(C(F)(F)F)cc2c1=O] | 539 [M + H]⁺ |
| 291C | 2-[1-[6-Fluoro-2-(1H- indol-2-yl)-4-oxo- chromen-8-yl]ethyl- amino]benzoic acid, Isomer 1 | 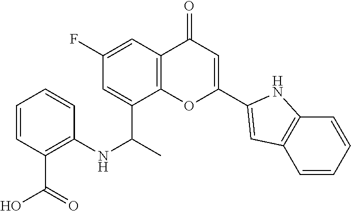 [SMILES: CC(Nc1ccccc1C(=O)O)c1cc(F)cc2c(=O)cc(-c3cc4ccccc4[nH]3)oc12] | 443 [M + H]⁺ |

### Intermediate 235A: 3-[[(R)-1-[3,6-Dimethyl-4-oxo-2-(3-pyridyl)chromen-8-yl]ethyl]amino]-6-fluoro-pyridine-2-carboxylic acid

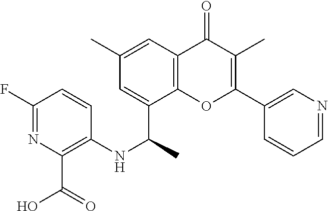 [SMILES: Cc1cc([C@@H](C)Nc2ccc(F)nc2C(=O)O)c2oc(-c3cccnc3)c(C)c(=O)c2c1]

A solution of tert-butyl 3-[[(1R)-1-[3,6-dimethyl-4-oxo-2-(3-pyridyl)chromen-8-yl]ethyl]amino]-6-fluoro-pyridine-2-carboxylate (0.23 g, 0.47 mmol) in DCM (5 mL) was treated with HCl (4 M in 1,4-dioxane) and allowed to stir for 1 h at rt under nitrogen. The reaction was concentrated to give the title compound (0.21 g) which was used without purification. MS ES+ m/z 434 [M+H]⁺.

### Intermediate 292C: 3-[[(1R)-1-[2-(5-tert-Butoxycarbonyl-6,7-dihydro-4H-pyrazolo[1,5-a]pyrazin-3-yl)-3,6-dimethyl-4-oxo-chromen-8-yl]ethyl]amino]-6-chloro-pyridine-2-carboxylic acid

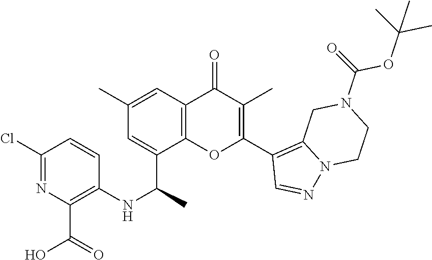 [SMILES: Cc1cc([C@@H](C)Nc2ccc(Cl)nc2C(=O)O)c2oc(-c3cnn4c3CN(C(=O)OC(C)(C)C)CC4)c(C)c(=O)c2c1]

A solution of 6-chloro-3-[[(1R)-1-[3,6-dimethyl-4-oxo-2-(4,5,6,7-tetrahydropyrazolo[1,5-a]pyrazin-3-yl)chromen-8-yl]ethyl]amino]pyridine-2-carboxylic acid (100 mg, 0.20 mmol) in 1,4-dioxane (5 mL) was treated with DIPEA (262 mg, 2.02 mmol) and di-tert-butyl decarbonate (66.3 mg, 0.30 mmol). The reaction was stirred at rt for 3 h. The reaction was washed with saturated aqueous NaHCO₃, saturated aqueous NH₄Cl, and then saturated aqueous NaCl. The organic layer was collected and concentrated under reduced pressure to give the title compound (180 mg, 90% purity, 130%) which was used without purification. ES/MS m/z 592 (M−H).

### Intermediate 293C: 2-[[(1R)-1-[3,6-Dimethyl-4-oxo-2-(3-pyridyl)chromen-8-yl]ethyl]amino]benzaldehyde

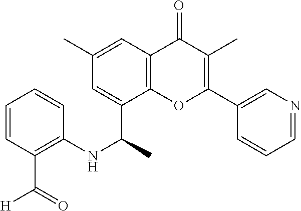 [SMILES: Cc1cc([C@@H](C)Nc2ccccc2C=O)c2oc(-c3cccnc3)c(C)c(=O)c2c1]

A vial was charged with 8-[(1R)-1-[2-(1,3-dioxolan-2-yl)anilino]ethyl]-3,6-dimethyl-2-(3-pyridyl)chromen-4-one (479 mg, 1.08 mmol), 1,4-dioxane (10 mL), and water (3 mL). The reaction was treated with HCl (5 M in 1,4-dioxane, 1 g, 5 mmol) and then stirred for 2 h at rt. The reaction was diluted with DCM and neutralized with 1M aqueous NaOH. After removal of the organic layer, the aqueous layer was extracted 3 times with DCM. The organic layers were combined, dried over Na₂SO₄, filtered, and concentrated under reduced pressure to give the title compound (400 mg, 93%) which was used without purification. ES/MS m/z 399 (M+H).

The following compound in Table 31 was made in a similar way as described for 2-[[(1R)-1-[3,6-dimethyl-4-oxo-2-(3-pyridyl)chromen-8-yl]ethyl]amino]benzaldehyde. Various methods were used to purify the compounds, which would be apparent to one skilled in the art.

TABLE 31

| | | | MS ES+ |
|---|---|---|---|
| Interm # | Chemical Name | Structure | m/z |
| 294C | 2-[[(1R)-1-(3,6-Dimethyl- 4-oxo-2-phenyl-chromen- 8-yl)ethyl]amino]benz- aldehyde | 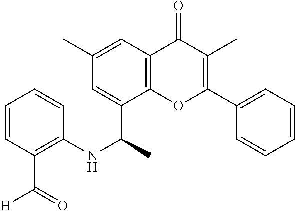 [SMILES: Cc1cc([C@@H](C)Nc2ccccc2C=O)c2oc(-c3ccccc3)c(C)c(=O)c2c1] | 398 [M + H]⁺ |

### Intermediate 236A: 2-[[(1R)-1-(3,6-Dimethyl-4-oxo-2-phenyl-chromen-8-yl)ethyl]amino]-6-fluoro-benzoic acid

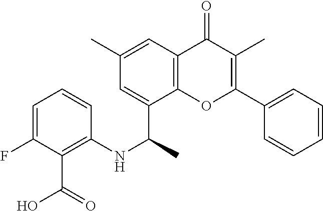 [SMILES: Cc1cc([C@@H](C)Nc2cccc(F)c2C(=O)O)c2oc(-c3ccccc3)c(C)c(=O)c2c1]

A solution of methyl 2-[[(1R)-1-(3,6-dimethyl-4-oxo-2-phenyl-chromen-8-yl)ethyl]amino]-6-fluoro-benzoate (0.67 g, 1.51 mmol) in THF (9 mL) and water (3 mL) was treated with lithium hydroxide (0.11 g, 4.53 mmol) and allowed to stir at rt overnight. Another 0.11 g of lithium hydroxide was added the reaction stirred at 50° C. for 6 h. An additional 2 eq of lithium hydroxide was added and the reaction stirred at 60° C. overnight. The reaction was allowed to cool to rt, diluted with DCM, washed with saturated aqueous ammonium chloride, and extracted twice with IPA/CHCl₃ (1:3). The organics were combined, dried over MgSO₄, filtered, and concentrated to give the title compound (0.76 g, 93%, 80% purity) as a white solid. MS ES+ m/z 432 [M+H]⁺.

The following compounds in Table 32 were made in a similar way as described for 2-[[(1R)-1-(3,6-dimethyl-4-oxo-2-phenyl-chromen-8-yl)ethyl]amino]-6-fluoro-benzoic acid. Various methods were used to purify the compounds, which would be apparent to one skilled in the art.

TABLE 32

| | | | MS ES+ |
|---|---|---|---|
| Interm # | Chemical Name | Structure | m/z |
| 295C | 4-[[(1R)-1-[3,6-Dimethyl- 2-(2-methylindazol-5-yl)- 4-oxo-chromen-8-yl]- ethyl]amino]-6-methyl-2- oxo-1H-pyridine-3-carbox- ylic acid | 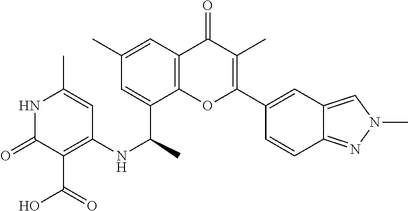 [SMILES: Cc1cc([C@@H](C)Nc2cc(C)[nH]c(=O)c2C(=O)O)c2oc(-c3ccc4nn(C)cc4c3)c(C)c(=O)c2c1] | 499 [M + H]⁺ |
| 296C | 2-[[(1R)-1-[3,6-Dimethyl- 2-(2-methylindazol-5-yl)- 4-oxo-chromen-8-yl]- ethyl]amino]-6-fluoro- benzoic acid | 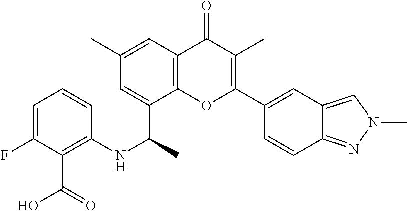 [SMILES: Cc1cc([C@@H](C)Nc2cccc(F)c2C(=O)O)c2oc(-c3ccc4nn(C)cc4c3)c(C)c(=O)c2c1] | 486 [M + H]⁺ |
| 297C | 6-Chloro-3-[[(1R)-1-[3,6- dimethyl-2-(2-methyl- pyrazolo[4,3-b]pyridin-5- yl)-4-oxo-chromen-8-yl]- ethyl]amino]pyridine-2- carboxylic acid | 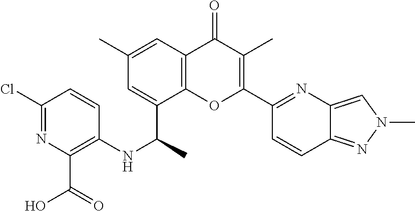 [SMILES: Cc1cc([C@@H](C)Nc2ccc(Cl)nc2C(=O)O)c2oc(-c3ccc4nn(C)cc4n3)c(C)c(=O)c2c1] | 504 [M + H]⁺ |
| 298C | 4-[[(1R)-1-(3,6-Dimethyl- 4-oxo-2-phenyl-chromen- 8-yl)ethyl]amino]-6-meth- yl-2-oxo-1H-pyridine-3- carboxylic acid | 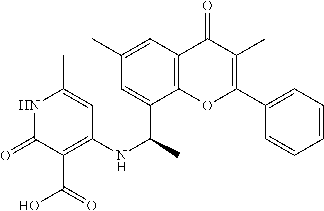 [SMILES: Cc1cc([C@@H](C)Nc2cc(C)[nH]c(=O)c2C(=O)O)c2oc(-c3ccccc3)c(C)c(=O)c2c1] | 445 [M + H]⁺ |

### Intermediate 26A: 3-[[(1R)-1-(3,6-Dimethyl-4-oxo-2-phenyl-chromen-8-yl)ethyl]amino]pyridine-2-carbohydrazide

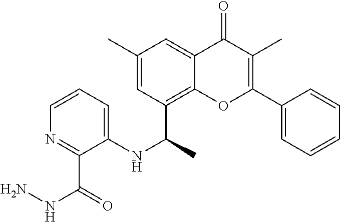 [SMILES: Cc1cc([C@@H](C)Nc2cccnc2C(=O)NN)c2oc(-c3ccccc3)c(C)c(=O)c2c1]

Cooled a solution of 3-[[(1R)-1-(3,6-dimethyl-4-oxo-2-phenyl-chromen-8-yl)ethyl]amino]pyridine-2-carboxylic acid (0.600 g, 1.45 mmol) in DCM (6 mL) to 0° C. Added thionyl chloride (1.45 mL, 2.90 mmol) and stirred the reaction at rt for 1 h. Added thionyl chloride (1.45 mL, 2.90 mmol) and stirred at 60° C. for 2 h. Concentrated to dryness, added DCM (6 mL) and triethylamine (0.61 mL, 4.34 mmol), and cooled to 0° C. Added N-BOC-hydrazine (0.48 g, 3.62 mmol) and stirred the reaction at rt for 1 h. Concentrated to dryness, added DCM (6 mL) and HCl (4M in 1,4-dioxane, 1.81 mL, 7.24 mmol), and stirred at rt for 3 days. Concentrated and purified by reverse phase chromatography eluted with a gradient of 0% to 100% ACN in aqueous NH₄HCO₃ (10 mM, plus 5% MeOH). Fractions containing product were diluted with saturated aqueous NaHCO₃ and EtOAc. The organics were dried over Na₂SO₄, filtered, and concentrated. Filtration from DCM/hexanes gave the title compound (0.36 g, 58%). MS ES+ m/z 429 [M+H]⁺.

### Intermediate 237A: 6-Chloro-3-[[(1R)-1-(3,6-dimethyl-4-oxo-2-phenyl-chromen-8-yl)ethyl]amino]pyridine-2-carbohydrazide

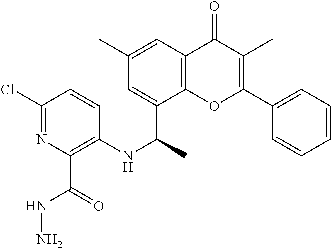 [SMILES: Cc1cc([C@@H](C)Nc2ccc(Cl)nc2C(=O)NN)c2oc(-c3ccccc3)c(C)c(=O)c2c1]

Combined 6-chloro-3-[[(1R)-1-(3,6-dimethyl-4-oxo-2-phenyl-chromen-8-yl)ethyl]amino]pyridine-2-carboxylic acid (0.40 g, 0.89 mmol), DIPEA (0.35 g, 2.67 mmol), 2,4,6-tripropyl-1,3,5-trioxa-2,4,6-triphosphinane-2,4,6-trioxide (1.69 g, 2.67 mmol), and hydrazine hydrate (0.21 g, 2.67 mmol) in DMA (4 mL) and stirred at 25° C. for 12 h. After 12 h, the reaction was treated with the same amount of DIPEA, 2,4,6-tripropyl-1,3,5-trioxa-2,4,6-triphosphinane-2,4,6-trioxide, and hydrazine hydrate and allowed to stir at rt. The reaction was purified by reversed phase chromatography eluted with 10% to 100% ACN in 0.1% aqueous formic acid to give the title compound (0.12 g, 29%). MS ES+ m/z 463 [M+H]⁺.

The following compounds in Table 33 were made in a similar way as described for 6-chloro-3-[[(1R)-1-(3,6-dimethyl-4-oxo-2-phenyl-chromen-8-yl)ethyl]amino]pyridine-2-carbohydrazide. Various methods were used to purify the compounds, which would be apparent to one skilled in the art.

TABLE 33

| | | | MS ES+ |
|---|---|---|---|
| Interm # | Chemical Name | Structure | m/z |
| 299C | 6-Chloro-3-[[(1R)-1- (2-ethylsulfanyl-3,6- dimethyl-4-oxo- chromen-8-yl)ethyl]- amino]-N-methylsul- fonyl-pyridine-2- carboxamide | 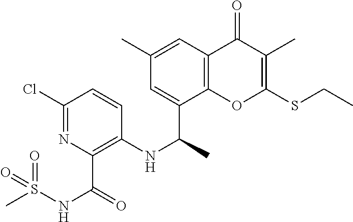 [SMILES: CCSc1oc2c([C@@H](C)Nc3ccc(Cl)nc3C(=O)NS(C)(=O)=O)cc(C)cc2c(=O)c1C] | 510 [M + H]⁺ |
| 300C | tert-Butyl 3-[8-[(1R)- 1-[[6-chloro-2-(meth- ylsulfonylcarbamoyl)- 3-pyridyl]amino]eth- yl]-3,6-dimethyl-4- oxo-chromen-2-yl]- 6,7-dihydro-4H- pyrazolo[1,5-a]pyr- azine-5-carboxylate | 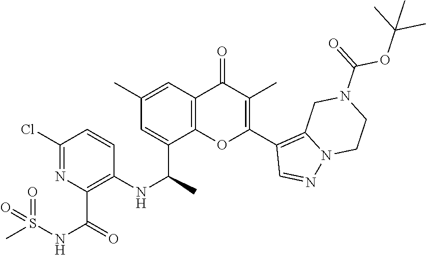 [SMILES: Cc1cc([C@@H](C)Nc2ccc(Cl)nc2C(=O)NS(C)(=O)=O)c2oc(-c3cnn4c3CN(C(=O)OC(C)(C)C)CC4)c(C)c(=O)c2c1] | 671 [M + H]⁺ |

### Intermediate 238A: tert-Butyl N-[[3-[[(1R)-1-[3,6-dimethyl-2-(2-methylindazol-5-yl)-4-oxo-chromen-8-yl]ethyl]amino]-6-methyl-pyridine-2-carbonyl]amino]carbamate

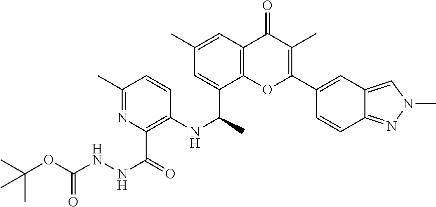 [SMILES: Cc1cc([C@@H](C)Nc2ccc(C)nc2C(=O)NNC(=O)OC(C)(C)C)c2oc(-c3ccc4nn(C)cc4c3)c(C)c(=O)c2c1]

A solution of 3-[[(1R)-1-[3,6-dimethyl-2-(2-methylindazol-5-yl)-4-oxo-chromen-8-yl]ethyl]amino]-6-methyl-pyridine-2-carboxylic acid (0.40 g, 0.83 mmol) in DMF (6 mL) was treated with HATU (0.95 g, 2.50 mmol) and DIPEA (0.54 g, 4.17 mmol). The mixture was stirred at rt for 5 min, treated with N-BOC hydrazine (0.33 g, 2.50 mmol), and stirred at rt overnight. The reaction was diluted with EtOAc and washed with 10% aqueous LiCl. The organic layer was washed with saturated aqueous sodium chloride, collected, dried over Na₂SO₄, filtered, and concentrated. The residue was purified by silica gel chromatography eluted with 0% to 10000 EtOAc/EtOH (3:1) in heptane to give the title compound (0.50 g, 91%, 91% purity) as a tan gel. MS ES+ m/z 597 [M+H]⁺.

The following compounds in Table 34 were made in a similar way as described for tert-butyl N-[[3-[[(1R)-1-[3,6-dimethyl-2-(2-methylindazol-5-yl)-4-oxo-chromen-8-yl]ethyl]amino]-6-methyl-pyridine-2-carbonyl]amino]carbamate. Various methods were used to purify the compounds, which would be apparent to one skilled in the art.

TABLE 34

| | | | MS ES+ |
|---|---|---|---|
| Interm # | Chemical Name | Structure | m/z |
| 239A¹ | tert-Butyl N-[3-[(1R)- 1-(3,6-dimethyl-4-oxo- 2-phenyl-chromen-8- yl)ethyl]amino]-6- methyl-pyridine-2- carbonyl]amino]car- bamate | 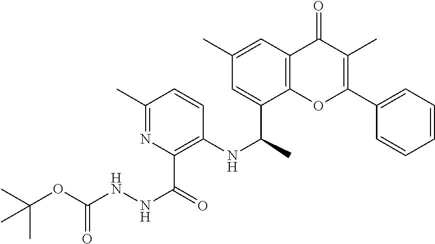 [SMILES: Cc1cc([C@@H](C)Nc2ccc(C)nc2C(=O)NNC(=O)OC(C)(C)C)c2oc(-c3ccccc3)c(C)c(=O)c2c1] | 543 [M + H]⁺ |
| 240A¹ | tert-Butyl N-[6-[(1R)- 1-(3,6-dimethyl-4-oxo- 2-phenyl-chromen-8- yl)ethyl]amino]-2,3- difluoro-benzoyl]- amino]carbamate | 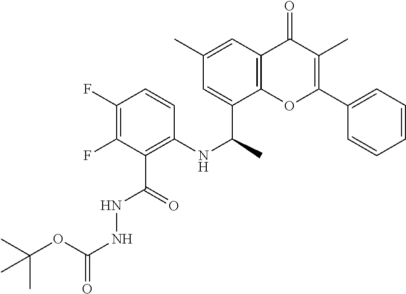 [SMILES: Cc1cc([C@@H](C)Nc2ccc(F)c(F)c2C(=O)NNC(=O)OC(C)(C)C)c2oc(-c3ccccc3)c(C)c(=O)c2c1] | 564 [M + H]⁺ |
| 241A | tert-butyl N-[[3-[(1R)- 1-[2-(3,4-difluoro- phenyl)-3,6-dimethyl- 4-oxo-chromen-8-yl]- ethyl]amino]-6-methyl- pyridine-2-carbonyl]- amino]carbamate | 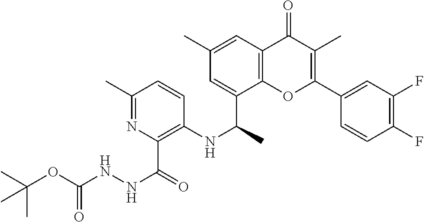 [SMILES: Cc1cc([C@@H](C)Nc2ccc(C)nc2C(=O)NNC(=O)OC(C)(C)C)c2oc(-c3ccc(F)c(F)c3)c(C)c(=O)c2c1] | 579 [M + H]⁺ |
| 242A¹ | tert-Butyl N-[[2-[(1R)- 1-(3,6-dimethyl-4-oxo- 2-phenyl-chromen-8- yl)ethyl]amino]-6- fluorobenzoyl]amino]- carbamate | 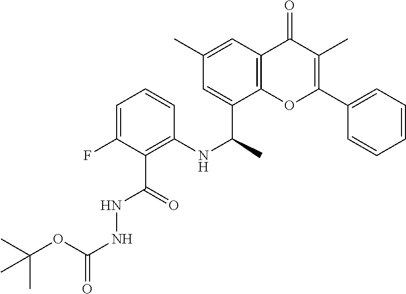 [SMILES: Cc1cc([C@@H](C)Nc2cccc(F)c2C(=O)NNC(=O)OC(C)(C)C)c2oc(-c3ccccc3)c(C)c(=O)c2c1] | 546 [M + H]⁺ |
| 243A¹ | tert-Butyl N-[[3-[(1R)- 1-[2-[6-(difluorometh- yl)-2-pyridyl]-3,6- dimethyl-4-oxo- chromen-8-yl]ethyl]- amino]pyridine-2-car- bonyl]amino]carbamate | 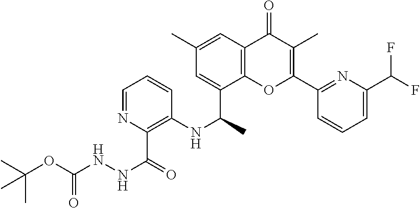 [SMILES: Cc1cc([C@@H](C)Nc2cccnc2C(=O)NNC(=O)OC(C)(C)C)c2oc(-c3cccc(C(F)F)n3)c(C)c(=O)c2c1] | 580 [M + H]⁺ |
| 301C | 6-Chloro-3-[[(1R)-1- [3,6-dimethyl-4-oxo-2- [3-(trifluoromethyl)-1- bicyclo[1.1.1]pentanyl] chromen-8-yl]ethyl]- amino]pyridine-2- carboxamide | 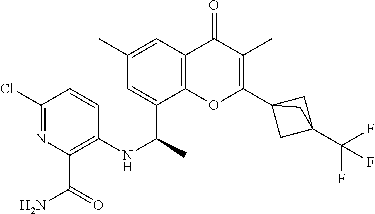 [SMILES: Cc1cc([C@@H](C)Nc2ccc(Cl)nc2C(N)=O)c2oc(C34CC(C(F)(F)F)(C3)C4)c(C)c(=O)c2c1] | 506 [M + H]⁺ |
| 302C | 2-[[(1R)-1-(3,6- Dimethyl-4-oxo-2- phenyl-chromen-8-yl)- ethyl]amino]benzamide | 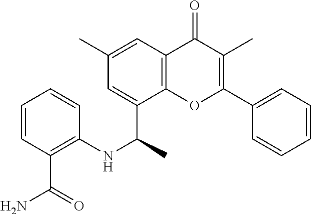 [SMILES: Cc1cc([C@@H](C)Nc2ccccc2C(N)=O)c2oc(-c3ccccc3)c(C)c(=O)c2c1] | 413 [M + H]⁺ |
| 303C | 6-Chloro-3-[1-[6- fluoro-3-methyl-2-(1- methylpyrazol-4-yl)-4- oxo-chromen-8-yl]- ethylamino]pyridine- 2-carboxamide, Isomer 1 | 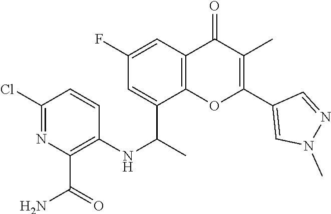 [SMILES: Cc1c(-c2cnn(C)c2)oc2c(C(C)Nc3ccc(Cl)nc3C(N)=O)cc(F)cc2c1=O] | 456 [M + H]⁺ |
| 304C | 6-Chloro-3-[[(1R)-1- (2-cyclopropyl-3,6- dimethyl-4-oxo- chromen-8-yl)ethyl]- amino]pyridine-2- carboxamide | 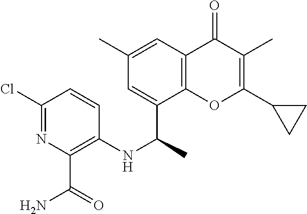 [SMILES: Cc1cc([C@@H](C)Nc2ccc(Cl)nc2C(N)=O)c2oc(C3CC3)c(C)c(=O)c2c1] | 412 [M + H]⁺ |

¹Material used without purification.

### Intermediate 244A: tert-Butyl N-[[3-[[(1R)-1-(3,6-dimethyl-4-oxo-2-phenyl-chromen-8-yl)ethyl]amino]pyridine-2-carbonyl]sulfamoyl]carbamate

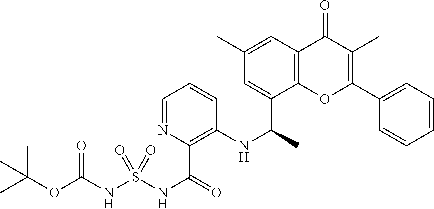 [SMILES: Cc1cc([C@@H](C)Nc2cccnc2C(=O)NS(=O)(=O)NC(=O)OC(C)(C)C)c2oc(-c3ccccc3)c(C)c(=O)c2c1]

A mixture of 3-[[(1R)-1-(3,6-dimethyl-4-oxo-2-phenyl-chromen-8-yl)ethyl]amino]pyridine-2-carboxylic acid (0.10 g, 0.24 mmol), 2-chloro-1-methyl-pyridin-1-ium iodide (92.5 mg, 0.36 mmol), tert-butyl sulfamoylcarbamate (94.7 mg, 0.48 mmol) and DMAP (2.95 mg, 0.02 mmol) were dissolved in DCM (2 mL) and allowed to stir. After 5 min, triethylamine (73.2 mg, 0.72 mmol) was added to the reaction and allowed to stir overnight at rt. The reaction was concentrated and the residue purified by reverse phase C18 flash chromatography eluted with ACN in water with 0.1% formic acid. Fractions containing the product were combined, washed with saturated aqueous sodium chloride, extracted with IPA/CHCl₃ (1:3), the organics collected, dried over MgSO₄, filtered, and concentrated to give the title compound (0.12 g, 83%) as a foam. MS ES+ m/z 591 [M−H]⁻.

The following compound in Table 35 was made in a similar way as described for tert-butyl N-[[3-[[(1R)-1-(3,6-dimethyl-4-oxo-2-phenyl-chromen-8-yl)ethyl]amino]pyridine-2-carbonyl]sulfamoyl]carbamate. Various methods were used to purify the compound, which would be apparent to one skilled in the art.

TABLE 35

| | | | MS ES+ |
|---|---|---|---|
| Interm # | Chemical Name | Structure | m/z |
| 305C | tert-Butyl N-[[6-chloro- 3-[[(1R)-1-(3,6-dimeth- yl-4-oxo-2-phenyl- chromen-8-yl)ethyl]- amino]pyridine-2- carbonyl]sulfamoyl]- carbamate | 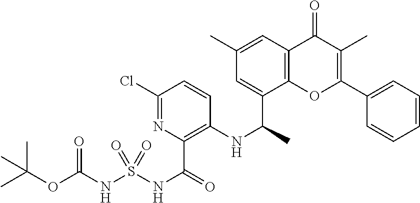 [SMILES: Cc1cc([C@@H](C)Nc2ccc(Cl)nc2C(=O)NS(=O)(=O)NC(=O)OC(C)(C)C)c2oc(-c3ccccc3)c(C)c(=O)c2c1] | 627 [M + H]⁺ |

### Intermediate 27A: Methyl 6-bromo-3-[[(1R)-1-(2-ethylsulfanyl-3,6-dimethyl-4-oxo-chromen-8-yl)ethyl]amino]pyridine-2-carboxylate

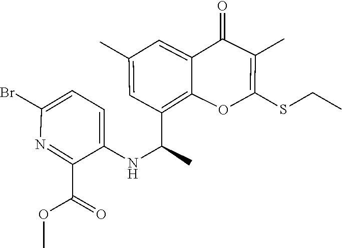 [SMILES: CCSc1oc2c([C@@H](C)Nc3ccc(Br)nc3C(=O)OC)cc(C)cc2c(=O)c1C]

Methyl 6-bromo-3-fluoropicolinate (0.25 g, 1.07 mmol) and 8-[(1R)-1-aminoethyl]-2-ethylsulfanyl-3,6-dimethyl-chromen-4-one (0.25 g, 0.89 mmol) were dissolved in DMSO (1 mL) and DIPEA (0.78 mL, 4.45 mmol) was added. The vial was sealed and the reaction heated to 120° C. for 24 h. Concentrated with a small amount of diatomaceous earth and purified by reverse phase chromatography eluted with a gradient of 10 to 70% ACN in aqueous NH₄HCO₃ (10 mM, plus 5% MeOH) to give the title compound (0.18 g, 40%). MS ES+ m/z 491 [M+H]⁺.

The following compound in Table 36 was made in a similar way as described for methyl 6-bromo-3-[[(1R)-1-(2-ethylsulfanyl-3,6-dimethyl-4-oxo-chromen-8-yl)ethyl]amino]pyridine-2-carboxylate.

TABLE 36

| | | | MS ES+ |
|---|---|---|---|
| Interm # | Chemical Name | Structure | m/z |
| 28A | Methyl 5-chloro-2-[(1R)- 1-(2-ethylsulfanyl-3,6- dimethyl-4-oxo-chromen- 8-yl)ethyl]amino]benzoate | 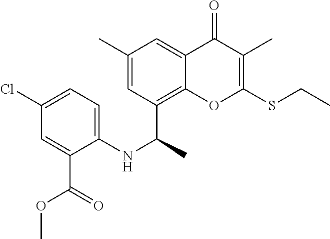 [SMILES: CCSc1oc2c([C@@H](C)Nc3ccc(Cl)cc3C(=O)OC)cc(C)cc2c(=O)c1C] | 447 [M + H]⁺ |
| 306C¹ | 6-Chloro-3-[[(1R)-1-[3,6- dimethyl-2-(1-methyl- pyrazolo[4,3-b]pyridin-5- yl)-4-oxo-chromen-8- yl]ethyl]amino]pyridine- 2-carbonitrile | 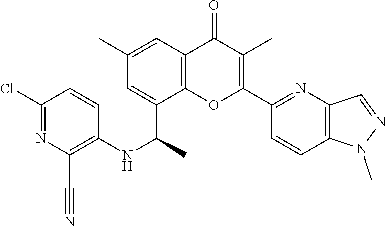 [SMILES: Cc1cc([C@@H](C)Nc2ccc(Cl)nc2C#N)c2oc(-c3ccc4c(cnn4C)n3)c(C)c(=O)c2c1] | 485 [M + H]⁺ |

¹ACN used as the solvent.

### Intermediate 307C: 8-[(1R)-1-[2-[2-[dimethyl(oxo)-lambda6-sulfanylidene]acetyl]anilino]ethyl]-3,6-dimethyl-2-phenyl-chromen-4-one

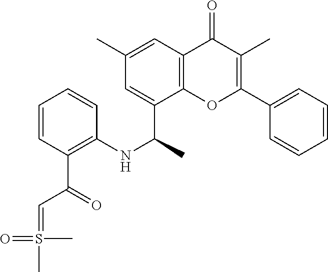 [SMILES: Cc1cc([C@@H](C)Nc2ccccc2C(=O)C=S(C)(C)=O)c2oc(-c3ccccc3)c(C)c(=O)c2c1]

A vial containing trimethylsulfoxonium iodide (291 mg, 1.32 mmol) was dried for 2 h at 65° C. and then treated with dry THF (5 mL) when cool. The reaction was treated with potassium tert-butoxide (148 mg, 1.32 mmol), the vial sealed, and the reaction stirred at 66° C. for 2 h. In a separate vial, 2-[[(1R)-1-(3,6-dimethyl-4-oxo-2-phenyl-chromen-8-yl)ethyl]amino]benzoic acid (455 mg, 1.10 mmol) was suspended in toluene (5.5 mL), treated dropwise with thionyl chloride (131 mg, 1.10 mmol), and the reaction stirred at 80° C. for 15 min. The second vial was allowed to cool to rt and concentrated under reduced pressure to give an acid chloride. Both vials were cooled in ice baths and the contents of the first vial were added via syringe to the acid chloride. The reaction was removed from the cooling bath and allowed to warm to rt. The reaction was not complete so was treated with a second batch of the first vial except that is was only stirred at 66° C. for 15 min. The reaction was quenched with water and the mixture concentrated under reduced pressure to remove the THF. The reaction was diluted with DCM, the layers separated, and the remaining aqueous layer re-extracted with fresh DCM. The combined organic layers were dried over MgSO₄, filtered, and concentrated under reduced pressure. The residue purified by silica gel chromatography eluted with 0% to 100% EtOAc in DCM and repurified by reversed phase chromatography on C18 eluted with 0% to 100% ACN in 10 mM aqueous NH₄HCO₃ (with 5% MeOH) to give the title compound. ES/MS m/z 488 (M+H).

### Intermediate 308C: 8-[(1R)-1-[2-(2-Chloroacetyl)anilino]ethyl]-3,6-dimethyl-2-phenyl-chromen-4-one

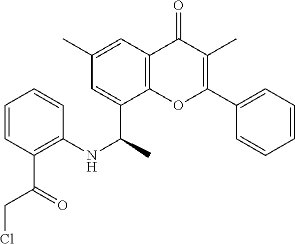 [SMILES: Cc1cc([C@@H](C)Nc2ccccc2C(=O)CCl)c2oc(-c3ccccc3)c(C)c(=O)c2c1]

A vial was charged with 8-[(1R)-1-[2-[2-[dimethyl(oxo)-lambda6-sulfanylidene]acetyl]anilino]ethyl]-3,6-dimethyl-2-phenyl-chromen-4-one and 1,4-dioxane (6.2 mL) and then treated with HCl in 1,4-dioxane (4 M, 227 mg, 6.21 mmol). The reaction was heated at 90° C. for 13 h. The reaction was concentrated under reduced pressure and the residue purified by reversed phase chromatography on C18 eluted with 0% to 100% ACN in 10 mM aqueous NH₄HCO₃ (with 5% MeOH) and repurified by silica gel chromatography eluted with 0% to 100% EtOAc in DCM to give the title compound (110.8 mg, 40%) as a yellow powder. ES/MS m/z 446 (M+H).

### Intermediate 309C: 6-Chloro-3-[[(1R)-1-[3,6-dimethyl-4-oxo-2-[3-(trifluoromethyl)-1-bicyclo[1.1.1]pentanyl]chromen-8-yl]ethyl]amino]pyridine-2-carbonitrile

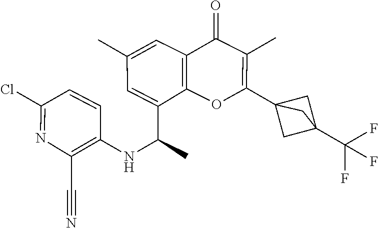 [SMILES: Cc1cc([C@@H](C)Nc2ccc(Cl)nc2C#N)c2oc(C34CC(C(F)(F)F)(C3)C4)c(C)c(=O)c2c1]

A solution of 6-chloro-3-[[(1R)-1-[3,6-dimethyl-4-oxo-2-[3-(trifluoromethyl)-1-bicyclo[1.1.1]pentanyl]chromen-8-yl]ethyl]amino]pyridine-2-carboxamide (97 mg, 0.19 mmol) in DCM (3 mL) was treated with triethylamine (0.29 g, 2.9 mmol) and then trifluoroacetic anhydride (0.20 g, 0.96 mmol) at 0° C. The reaction was warmed to rt and stirred for 2 h. The reaction was diluted with EtOAc and washed with saturated aqueous NaHCO₃. The organic layer was collected, dried over Na₂SO₄, filtered, and concentrated under reduced pressure. The residue was purified by silica gel chromatography eluted with 0% to 100% EtOAc in heptane to give the title compound (192 mg, 50% purity, 100%) as a yellow gel. ES/MS m/z 488 (M+H).

The following compound in Table 37 was made in a similar way as described for 6-chloro-3-[[(1R)-1-[3,6-dimethyl-4-oxo-2-[3-(trifluoromethyl)-1-bicyclo[1.1.1]pentanyl]chromen-8-yl]ethyl]amino]pyridine-2-carbonitrile. Various methods were used to purify the compounds, which would be apparent to one skilled in the art.

TABLE 37

| | | | MS ES+ |
|---|---|---|---|
| Interm # | Chemical Name | Structure | m/z |
| 310C | 6-Chloro-3-[1-[6-fluoro-3- methyl-2-(1-methylpyrazol- 4-yl)-4-oxo-chromen-8-yl]- ethylamino]pyridine-2- carbonitrile, Isomer 1 | 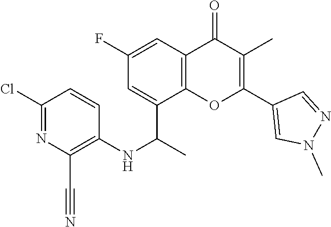 [SMILES: Cc1c(-c2cnn(C)c2)oc2c(C(C)Nc3ccc(Cl)nc3C#N)cc(F)cc2c1=O] | 438 [M + H]⁺ |
| 311C | 6-Chloro-3-[[(1R)-1-(2- cyclopropyl-3,6-dimethyl- 4-oxo-chromen-8-yl)- ethyl]amino]pyridine-2- carbonitrile | 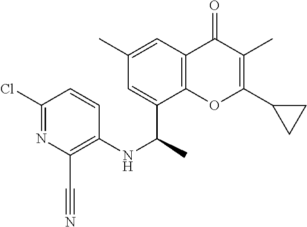 [SMILES: Cc1cc([C@@H](C)Nc2ccc(Cl)nc2C#N)c2oc(C3CC3)c(C)c(=O)c2c1] | 394 [M + H]⁺ |

### Intermediate 29A: 8-(1-Hydroxyethyl)-3,6-dimethyl-2-phenyl-chromen-4-one

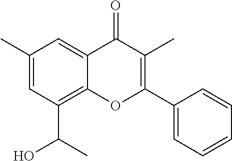 [SMILES: Cc1cc(C(C)O)c2oc(-c3ccccc3)c(C)c(=O)c2c1]

Dissolved 8-acetyl-3,6-dimethyl-2-phenyl-chromen-4-one (5.0 g, 17 mmol) in MeOH (6 mL) and DCM (6 mL). Cooled to −10° C., added NaBH₄ (0.78 g, 21 mmol), and stirred for 30 min. Added water (50 mL) and extracted with DCM (2×60 mL). The combined organic extracts were dried over anhydrous Na₂SO₄, concentrated, and purified by silica gel chromatography eluted with a gradient of 0 to 5% MeOH in DCM to give the title compound (4.2 g, 83%). MS ES+ m/z 295 [M+H]⁺.

### Intermediate 30A: 8-(1-Bromoethyl)-3,6-dimethyl-2-phenyl-chromen-4-one

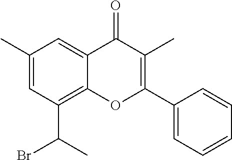 [SMILES: Cc1cc(C(C)Br)c2oc(-c3ccccc3)c(C)c(=O)c2c1]

Combined 8-(1-chloroethyl)-3,6-dimethyl-2-phenyl-chromen-4-one (2.0 g, 6.8 mmol) in DCM (25 mL). Cooled to 0° C., added PBr₃ (1.3 mL, 14 mmol) dropwise, and stirred the reaction for 2 h at rt. Slowly added saturated aqueous bicarbonate, then separated the layers. The organics were dried over anhydrous Na₂SO₄ and concentrated to give the title compound (1.4 g, 58%). MS ES+ m/z 359 [M+H]⁺.

### Intermediate 31A: 8-[(1R)-1-Hydroxyethyl]-3,6-dimethyl-2-phenyl-chromen-4-one

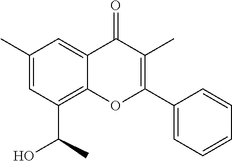 [SMILES: Cc1cc([C@@H](C)O)c2oc(-c3ccccc3)c(C)c(=O)c2c1]

A solution of 8-acetyl-3,6-dimethyl-2-phenyl-chromen-4-one (10 g, 34.2 mmol) and RuCl(p-cymene)[(R,R)-Ts-DPEN] (CAS 192139-92-7, 0.653 g, 1.03 mmol) in MeOH (50 mL) was stirred at 0-5° C. 1,8-Diazabicyclo[5.4.0]undec-7-ene (15.3 mL, 103 mmol) was added slowly, maintaining the temperature below 25° C. Formic acid (3.87 mL, 103 mmol) was added in portions, maintaining the temperature below 15° C. The reaction was stirred at 55° C. for 3 h, then cooled to rt. 4M aqueous HCl (50 mL) was slowly added. The resulting slurry was stirred overnight, filtered, and washed with water to give the title compound (9.9 g, 93%, 87% ee).

### Intermediate 32A: 8-[(1S)-1-Chloroethyl]-3,6-dimethyl-2-phenyl-chromen-4-one

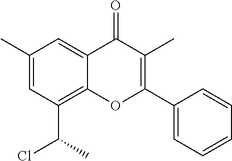 [SMILES: Cc1cc([C@H](C)Cl)c2oc(-c3ccccc3)c(C)c(=O)c2c1]

A solution of 8-[(1R)-1-hydroxyethyl]-3,6-dimethyl-2-phenyl-chromen-4-one (20 g, 68.0 mmol) in cyclopentyl methyl ether (200 mL) was stirred at room temperature. 2,4,6-Trichloro[1,3,5]triazine (12.5 g, 68.0 mmol) was added, followed by DMF (7.9 mL, 102 mmol). The reaction was stirred at room temperature for 14 h. Diluted with 2M aqueous sodium hydroxide (100 mL) and separated the layers. The organics were washed with water (100 mL)/saturated aqueous sodium bicarbonate (100 mL) and 5% aqueous lithium chloride (100 mL). The organics were concentrated and diluted with isopropanol (120 mL). The slurry was heated at 45° C. for 2 h and cooled to room temperature. Water (80 mL) was slowly added. Filtered to give the title compound (18.4 g, 87%, 94% ee). MS ES+ m/z 313 [M+H]⁺.

### Intermediate 245A: 8-[(1R)-1-(2-Iodoanilino)ethyl]-3,6-dimethyl-2-phenyl-chromen-4-one

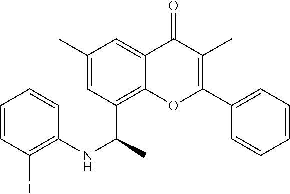 [SMILES: Cc1cc([C@@H](C)Nc2ccccc2I)c2oc(-c3ccccc3)c(C)c(=O)c2c1]

8-[(1S)-1-Chloroethyl]-3,6-dimethyl-2-phenyl-chromen-4-one (0.60 g, 1.92 mmol), 2-iodoaniline (1.26 g, 5.75 mmol), and DIPEA (1.24 g, 9.59 mmol) were combined in IPA (6 mL) and stirred at 60° C. overnight. The reaction was recharged twice with 1 g of 2-iodoaniline and 0.5 mL of DIPEA over the course of 8 h before stirring overnight. The reaction was concentrated and purified by silica gel chromatography eluted with 0% to 100% EtOAc in heptane to give the title compound (0.70 g, 74%). MS ES+ m/z 496 [M+H]⁺.

### Intermediate 246A: tert-Butyl 2-[[(1R)-1-(3-iodo-6-methyl-4-oxo-2-phenyl-chromen-8-yl)ethyl]amino]benzoate

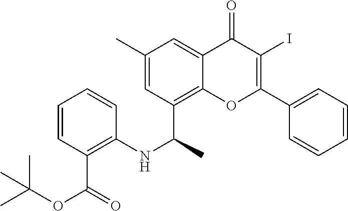 [SMILES: Cc1cc([C@@H](C)Nc2ccccc2C(=O)OC(C)(C)C)c2oc(-c3ccccc3)c(I)c(=O)c2c1]

A dry argon flushed flask was charged with tert-butyl 2-[[(1R)-1-(6-methyl-4-oxo-2-phenyl-chromen-8-yl)ethyl]amino]benzoate (1.40 g, 3.06 mmol) and THF (5 mL). The resulting solution was cooled to 0° C. and treated with 2,2,6,6-tetramethylpiperidinyzinc chloride lithium chloride complex solution (3.49 g, 1M, 12.30 mmol) dropwise over 30 min and then the reaction was allowed to stir at 25° C. for 7 h. Another 6 mL of 2,2,6,6-tetramethylpiperidinyzinc chloride lithium chloride complex solution was added and the reaction stirred overnight at rt. The reaction was cooled to 0° C. and treated with a freshly prepared solution of iodine (1.56 g, 1M in THF, 6.15 mmol) dropwise over 30 min. The reaction was stirred at 0° C. for 1 h and then at rt for 12 h. The reaction was cooled to −40° C. and quenched with MeOH. The reaction was diluted with ammonium chloride/ammonia solution (50 mL, 2M in water) and extracted with DCM (3×300 mL). The combined organics were washed with aqueous Na₂CO₃ (50 mL), collected, dried over Na₂SO₄, filtered, and concentrated. The residue was purified by silica gel chromatography eluted with 0% to 10% EtOAc in heptane to give the title compound (1.64 g, 87%). MS ES+ m/z 582 [M+H]⁺.

The following compounds in Table 38 was made in a similar way as described for tert-butyl 2-[[(1R)-1-(3-iodo-6-methyl-4-oxo-2-phenyl-chromen-8-yl)ethyl]amino]benzoate. Various methods were used to purify the compounds, which would be apparent to one skilled in the art.

TABLE 38

| | | | MS ES+ |
|---|---|---|---|
| Interm # | Chemical Name | Structure | m/z |
| 312C | 6-Chloro-3-[(1R)-1-(3-iodo- 6-methyl-4-oxo-2-phenyl- chromen-8-yl)ethyl]amino]- pyridine-2-carboxylic acid | 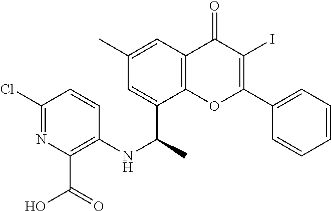 [SMILES: Cc1cc([C@@H](C)Nc2ccc(Cl)nc2C(=O)O)c2oc(-c3ccccc3)c(I)c(=O)c2c1] | 561 [M + H]⁺ |
| 313C | tert-Butyl 6-chloro-3-[(1R)- 1-(3-iodo-6-methyl-4-oxo- 2-phenyl-chromen-8-yl)- ethyl]amino]pyridine-2- carboxylate | 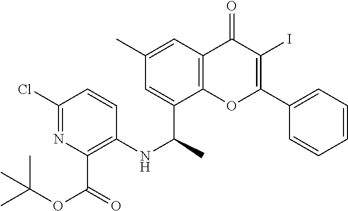 [SMILES: Cc1cc([C@@H](C)Nc2ccc(Cl)nc2C(=O)OC(C)(C)C)c2oc(-c3ccccc3)c(I)c(=O)c2c1] | 561 [MH − tBu]⁺ |
| 314C | 6-Chloro-3-[(1R)-1-[3-iodo- 6-methyl-4-oxo-2-(2- pyridyl)chromen-8-yl]- ethyl]amino]pyridine-2- carbonitrile | 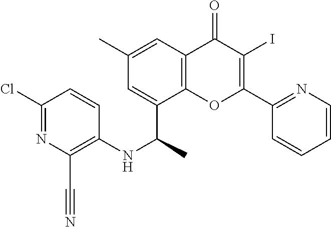 [SMILES: Cc1cc([C@@H](C)Nc2ccc(Cl)nc2C#N)c2oc(-c3ccccn3)c(I)c(=O)c2c1] | 543 [M + H]⁺ |
| 315C | 8-[(1R)-1-[(6-Chloro-2- methyl-3-pyridyl)amino]- ethyl]-3-iodo-6-methyl-2- (3-pyridyl)chromen-4-one | 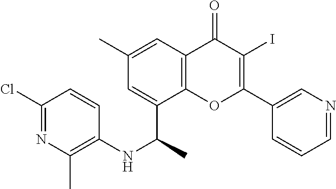 [SMILES: Cc1cc([C@@H](C)Nc2ccc(Cl)nc2C)c2oc(-c3cccnc3)c(I)c(=O)c2c1] | 532 [M + H]⁺ |
| 316C | 8-[(1R)-1-[[2-(2-Fluoro- phenyl)-3-pyridyl]amino]- ethyl]-3-iodo-6-methyl-2- (3-pyridyl)chromen-4-one | 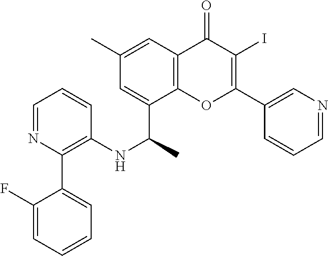 [SMILES: Cc1cc([C@@H](C)Nc2cccnc2-c2ccccc2F)c2oc(-c3cccnc3)c(I)c(=O)c2c1] | 578 [M + H]⁺ |

### Intermediate 317C: N-tert-Butyl-6-chloro-3-[[(1R)-1-(3-iodo-6-methyl-4-oxo-2-phenyl-chromen-8-yl)ethyl]amino]pyridine-2-sulfonamide

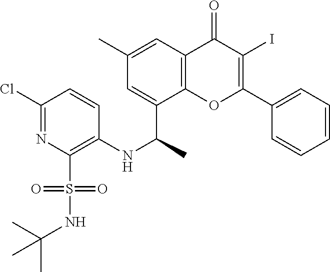 [SMILES: Cc1cc([C@@H](C)Nc2ccc(Cl)nc2S(=O)(=O)NC(C)(C)C)c2oc(-c3ccccc3)c(I)c(=O)c2c1]

A solution of N-tert-butyl-6-chloro-3-[[(1R)-1-(6-methyl-4-oxo-2-phenyl-chromen-8-yl)ethyl]amino]pyridine-2-sulfonamide (1.1 g, 2.09 mmol) in THF (10 mL) was cooled to −40° C. under a nitrogen atmosphere and treated with 2,2,6,6-tetramethylpiperdinylmagnesium chloride lithium chloride complex (13 mL, 12.55 mmol, 1 M in THF). The reaction was stirred at −10° C. for 2 h. A THF solution of iodine (4 mL, 4.18 mmol) was added dropwise over 30 min at −40° C. and then stirred at rt for 2 h. The reaction was quenched with saturated aqueous NH₄Cl at 0° C. The reaction was extracted with EtOAc (3×100 mL). The combined organics were washed with brine (100 mL), collected, dried over Na₂SO₄, filtered, and concentrated under reduced pressure. The residue was purified by silica gel chromatography eluted with 50% EtOAc in petroleum ether to give the title compound (0.30 g, 22%) as a yellow solid. ES/MS m/z 652 (M+H).

### Intermediate 383C: 3-[[(1R)-1-[3-Bromo-6-methyl-2-(1-methylpyrazol-4-yl)-4-oxo-chromen-8-yl]ethyl]amino]-N-tert-butyl-6-chloro-pyridine-2-sulfonamide

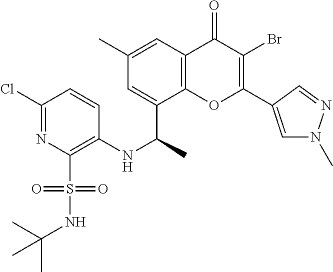 [SMILES: Cc1cc([C@@H](C)Nc2ccc(Cl)nc2S(=O)(=O)NC(C)(C)C)c2oc(-c3cnn(C)c3)c(Br)c(=O)c2c1]

A mixture of N-tert-butyl-6-chloro-3-[[(1R)-1-[6-methyl-2-(1-methylpyrazol-4-yl)-4-oxo-chromen-8-yl]ethyl]amino]pyridine-2-sulfonamide (400 mg, 0.75 mmol) and N-bromosuccinimide (134 mg, 0.75 mmol) in ACN was stirred at rt for 1 h. The reaction was concentrated under reduced pressure and the residue purified by reversed phase chromatography on C18 eluted with 65% to 70% ACN in water (with 0.1% NH₄OH) to give the title compound (250 mg, 54%) as a colorless solid. ES/MS m/z 608/610 (M+H).

### Intermediate 384C: N-tert-Butyl-6-chloro-3-[[(1R)-1-(3-cyano-6-methyl-4-oxo-2-phenyl-chromen-8-yl)ethyl]amino]pyridine-2-sulfonamide

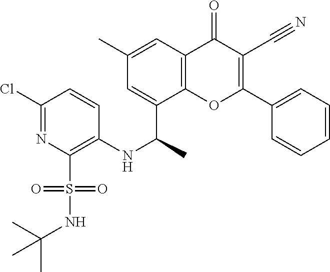 [SMILES: Cc1cc([C@@H](C)Nc2ccc(Cl)nc2S(=O)(=O)NC(C)(C)C)c2oc(-c3ccccc3)c(C#N)c(=O)c2c1]

A solution of N-tert-butyl-6-chloro-3-[[(1R)-1-(3-iodo-6-methyl-4-oxo-2-phenyl-chromen-8-yl)ethyl]amino]pyridine-2-sulfonamide (180 mg, 0.28 mmol) in NMP (4 mL) was treated with copper(I) cyanide (124 mg, 1.38 mmol) at rt. The reaction was stirred overnight at 120° C. under a nitrogen atmosphere. The reaction was cooled to rt, diluted with water (50 mL), and extracted with EtOAc (3×50 mL). The combined organics were washed with saturated aqueous NaCl (100 mL), collected, dried over Na₂SO₄, filtered and concentrated under reduced pressure. The residue was purified by reversed phase chromatography on C18 eluted with 80% to 95% ACN in water to give the title compound (73 mg, 48%) as a light yellow solid. ES/MS m/z 551 (M+H).

### Intermediate 247A: tert-Butyl 2-[[(1R)-1-(6-methyl-4-oxo-2-phenyl-3-thiazol-5-yl-chromen-8-yl)ethyl]amino]benzoate

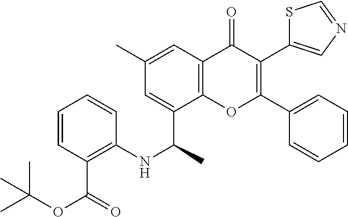 [SMILES: Cc1cc([C@@H](C)Nc2ccccc2C(=O)OC(C)(C)C)c2oc(-c3ccccc3)c(-c3cncs3)c(=O)c2c1]

Charged a tube with tert-butyl 2-[[(1R)-1-(3-iodo-6-methyl-4-oxo-2-phenyl-chromen-8-yl)ethyl]amino]benzoate (300 mg, 0.52 mmol), 1,1′-bis(diphenylphophino)ferrocene-palladium(II) dichloride (75.5 mg, 0.10 mmol), cuprous chloride (51 mg, 0.52 mmol), sodium carbonate (109 mg, 1.03 mmol), 5-(4,4,5,5-tetramethyl-1,3,2-dioxaborolan-2-yl)thiazole (436 mg, 2.06 mmol) and toluene (15 mL). The reaction was heated at 80° C. under a nitrogen atmosphere for 20 h. The reaction was recharged and allowed to stir at 80° C. overnight. The reaction was allowed to cool, filtered through celite, concentrated, and the residue purified by silica gel chromatography eluted with 0% to 100% EtOAc in heptane to give the title compound (208 mg, 75%). MS ES+ m/z 539 [M+H]⁺.

### Intermediate 318C: N-tert-Butyl-6-chloro-3-[[(1R)-1-(3-isoxazol-4-yl-6-methyl-4-oxo-2-phenyl-chromen-8-yl)ethyl]amino]pyridine-2-sulfonamide

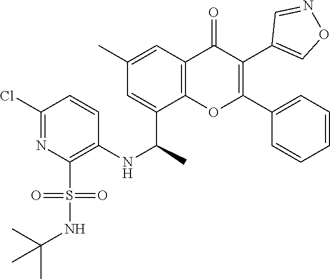 [SMILES: Cc1cc([C@@H](C)Nc2ccc(Cl)nc2S(=O)(=O)NC(C)(C)C)c2oc(-c3ccccc3)c(-c3cnoc3)c(=O)c2c1]

A solution of N-tert-butyl-6-chloro-3-[[(1R)-1-(3-iodo-6-methyl-4-oxo-2-phenyl-chromen-8-yl)ethyl]amino]pyridine-2-sulfonamide (80 mg, 0.12 mmol) and 4-(4,4,5,5-tetramethyl-1,3,2-dioxaborolan-2-yl)isoxazole (48 mg, 0.25 mmol) in 1,4-dioxane (2 mL) and water (0.2 mL) was treated with potassium carbonate (34 mg, 0.25 mmol) and 1,1′-bis (di-t-butylphosphino)ferrocene palladium dichloride (16 mg, 0.03 mmol) at rt under a nitrogen atmosphere. The reaction was stirred at 80° C. for 4 h. After cooling to rt, the reaction was concentrated under reduced pressure and the residue purified by silica gel chromatography eluted with 33% EtOAc in petroleum ether to give the title compound (50 mg, 69%) as a light yellow solid. ES/MS m/z 593 (M+H).

### Intermediate 385C: N-tert-Butyl-6-chloro-3-[[(1R)-1-[3-isoxazol-4-yl-6-methyl-2-(1-methylpyrazol-4-yl)-4-oxo-chromen-8-yl]ethyl]amino]pyridine-2-sulfonamide

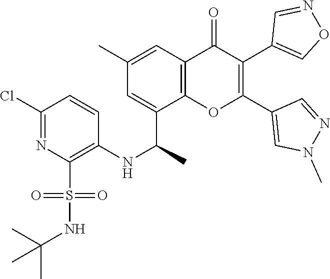 [SMILES: Cc1cc([C@@H](C)Nc2ccc(Cl)nc2S(=O)(=O)NC(C)(C)C)c2oc(-c3cnn(C)c3)c(-c3cnoc3)c(=O)c2c1]

A mixture of 3-[[(1R)-1-[3-bromo-6-methyl-2-(1-methylpyrazol-4-yl)-4-oxo-chromen-8-yl]ethyl]amino]-N-tert-butyl-6-chloro-pyridine-2-sulfonamide (190 mg, 0.31 mmol), triethylamine (63.2 mg, 0.62 mmol), 1,1′-bis (di-t-butylphosphino)ferrocene palladium dichloride (20.34 mg, 0.03 mmol), and 4-(4,4,5,5-tetramethyl-1,3,2-dioxaborolan-2-yl)isoxazole (121.71 mg, 0.62 mmol) in THF (2 mL) and water (0.5 mL) was stirred overnight at rt under a nitrogen atmosphere. The reaction was filtered and the solids washed with THF (2×10 mL). The filtrate was concentrated under reduced pressure and the residue purified by reversed phase chromatography on C18 eluted with 75% to 85% ACN in water (with 0.1% NH₄OH) to give the title compound (92 mg, 46%) as a colorless solid. ES/MS m/z 597 (M+H).

### Intermediate 248A: 6-Chloro-3-[[(1R)-1-(3-iodo-6-methyl-4-oxo-2-phenyl-chromen-8-yl)ethyl]amino]pyridine-2-carboxylic acid

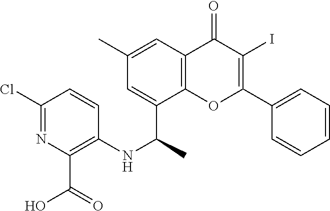 [SMILES: Cc1cc([C@@H](C)Nc2ccc(Cl)nc2C(=O)O)c2oc(-c3ccccc3)c(I)c(=O)c2c1]

A THF (3 mL) solution of tert-butyl 6-chloro-3-[[(1R)-1-(6-methyl-4-oxo-2-phenyl-chromen-8-yl)ethyl]amino]pyridine-2-carboxylate (0.47 g, 0.96 mmol) was treated with 2,2,6,6-tetramethylpiperidinyzinc chloride lithium chloride complex solution (0.68 mg, 1 M, 2.39 mmol) and allowed to stir at rt for 3 h. The reaction was quenched with iodine (0.97 g, 3.83 mmol) dissolved in THF, and the reaction allowed to stir for 1 h. The reaction was diluted with 20 mL of EtOAc/water. The organic layer was collected, dried over MgSO₄, filtered, and concentrated. The residue was purified by reversed phase chromatography eluted with 10% to 100% ACN (with 0.1% formic acid) in water (with 0.1% formic acid) to give the title product (0.20 g, 37%). MS ES+ m/z 561 [M+H]⁺.

### Intermediate 33A: 3-[[(1R)-1-(3,6-Dimethyl-4-oxo-2-phenyl-chromen-8-yl)ethyl]amino]-N′-hydroxy-pyridine-2-carboxamidine

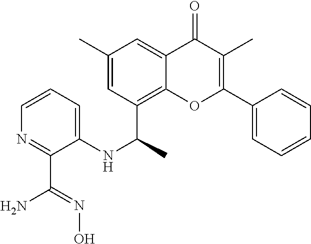 [SMILES: Cc1cc([C@@H](C)Nc2cccnc2/C(N)=N/O)c2oc(-c3ccccc3)c(C)c(=O)c2c1]

To a solution of 3-[[(1R)-1-(3,6-dimethyl-4-oxo-2-phenyl-chromen-8-yl)ethyl]amino]pyridine-2-carbonitrile (0.242 g, 0.612 mmol) in ethanol (5 mL) was added triethylamine (0.26 mL, 1.84 mmol) and hydroxylamine hydrochloride (0.085 g, 1.22 mmol). The reaction was heated at 80° C. for 18 h. Concentrated and purified by reverse phase chromatography eluted with a gradient of 0 to 100% ACN in H₂O+0.1% formic acid to give the title compound (0.26 g, 89%). MS ES+ m/z 429 [M+H]⁺.

The following compounds in Table 39 were made in a similar way as described for 3-[[(1R)-1-(3,6-dimethyl-4-oxo-2-phenyl-chromen-8-yl)ethyl]amino]-N′-hydroxy-pyridine-2-carboxamidine. Various methods were used to purify the compounds, which would be apparent to one skilled in the art.

TABLE 39

| | | | MS ES+ |
|---|---|---|---|
| Interm # | Chemical Name | Structure | m/z |
| 34A | 3-[[(1R)-1-(3,6-Dimethyl-4- oxo-2-phenyl-chromen-8- yl)ethyl]amino]-N′-hydroxy- 6-methyl-pyridine-2-carbox- amidine | 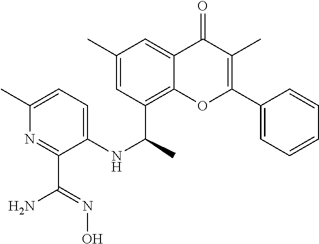 [SMILES: Cc1cc([C@@H](C)Nc2ccc(C)nc2/C(N)=N/O)c2oc(-c3ccccc3)c(C)c(=O)c2c1] | 443 [M + H]⁺ |
| 35A | 3-[[(1R)-1-[3,6-Dimethyl-4- oxo-2-(3-pyridyl)chromen- 8-yl]ethyl]amino]-N′- hydroxy-pyridine-2-car- boxamidine | 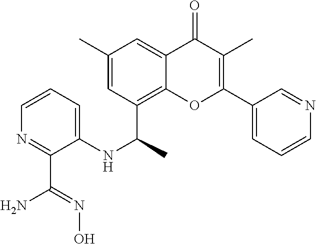 [SMILES: Cc1cc([C@@H](C)Nc2cccnc2/C(N)=N/O)c2oc(-c3cccnc3)c(C)c(=O)c2c1] | 430 [M + H]⁺ |
| 249A | 6-Chloro-3-[(1R)-1-[3,6- dimethyl-4-oxo-2-(2- pyridyl)chromen-8-yl]- ethyl]amino]-N′-hydroxy- pyridine-2-carboxamidine | 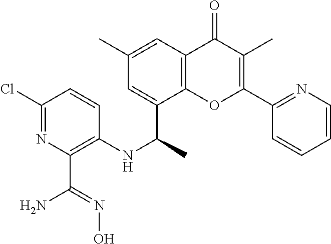 [SMILES: Cc1cc([C@@H](C)Nc2ccc(Cl)nc2/C(N)=N/O)c2oc(-c3ccccn3)c(C)c(=O)c2c1] | 464 [M + H]⁺ |
| 250A | 6-Chloro-3-[(1R)-1-(3,6- dimethyl-4-oxo-2-phenyl- chromen-8-yl)ethyl]amino]- N′-hydroxy-pyridine-2- carboxamidine | 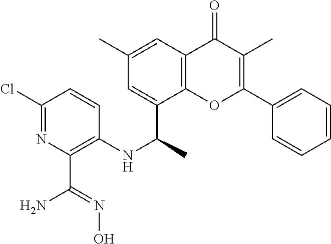 [SMILES: Cc1cc([C@@H](C)Nc2ccc(Cl)nc2/C(N)=N/O)c2oc(-c3ccccc3)c(C)c(=O)c2c1] | 463 [M + H]⁺ |
| 251A | 6-[[(1R)-1-(3,6-Dimethyl- 4-oxo-2-phenyl-chromen- 8-yl)ethyl]amino]-2,3- difluoro-N′-hydroxy- benzamidine | 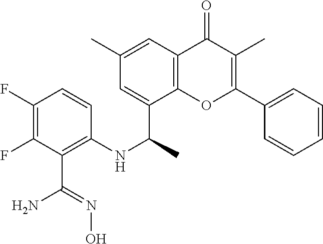 [SMILES: Cc1cc([C@@H](C)Nc2ccc(F)c(F)c2/C(N)=N/O)c2oc(-c3ccccc3)c(C)c(=O)c2c1] | 464 [M + H]⁺ |
| 252A | 6-Chloro-3-[[(1R)-1-[2-(3,4- difluorophenyl)-3,6-dimeth- yl-4-oxo-chromen-8-yl]- ethyl]amino]-N′-hydroxy- pyridine-2-carboxamidine | 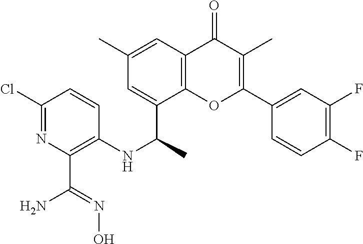 [SMILES: Cc1cc([C@@H](C)Nc2ccc(Cl)nc2/C(N)=N/O)c2oc(-c3ccc(F)c(F)c3)c(C)c(=O)c2c1] | 499 [M + H]⁺ |
| 253A | 6-Chloro-3-[(1R)-1-[2-(5- fluoro-3-pyridyl)-3,6- dimethyl-4-oxo-chromen-8- yl]ethyl]amino]-N′-hydroxy- pyridine-2-carboxamidine | 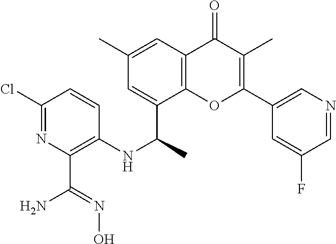 [SMILES: Cc1cc([C@@H](C)Nc2ccc(Cl)nc2/C(N)=N/O)c2oc(-c3cncc(F)c3)c(C)c(=O)c2c1] | 482 [M + H]⁺ |
| 254A | 2-Fluoro-6-[(1R)-1-[2-(3- fluorophenyl)-3,6-dimethyl- 4-oxo-chromen-8-yl]- ethyl]amino]-N′-hydroxy- benzamidine | 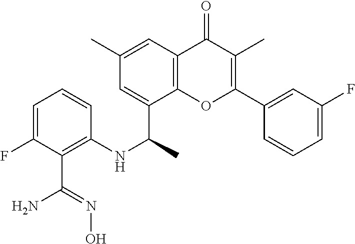 [SMILES: Cc1cc([C@@H](C)Nc2cccc(F)c2/C(N)=N/O)c2oc(-c3cccc(F)c3)c(C)c(=O)c2c1] | 464 [M + H]⁺ |
| 255A | 2,3-Difluoro-6-[(1R)-1-[2- (5-fluoro-3-pyridyl)-3,6- dimethyl-4-oxo-chromen-8- yl]ethyl]amino]-N′-hydroxy- benzamidine | 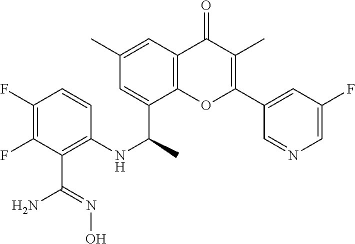 [SMILES: Cc1cc([C@@H](C)Nc2ccc(F)c(F)c2/C(N)=N/O)c2oc(-c3cncc(F)c3)c(C)c(=O)c2c1] | 483 [M + H]⁺ |
| 256A | 2-[[(1R)-1-(3,6-Dimethyl-4- oxo-2-phenyl-chromen-8- yl)ethyl]amino]-6-fluoro-N′- hydroxy-benzamidine | 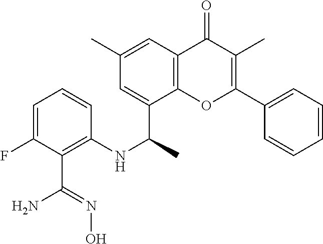 [SMILES: Cc1cc([C@@H](C)Nc2cccc(F)c2/C(N)=N/O)c2oc(-c3ccccc3)c(C)c(=O)c2c1] | 446 [M + H]⁺ |
| 257A | 2,3-Difluoro-6-[(1R)-1-[2- (2-fluorophenyl)-3,6- dimethyl-4-oxo-chromen- 8-ylethyl]amino]-N′- hydroxy-benzamidine | 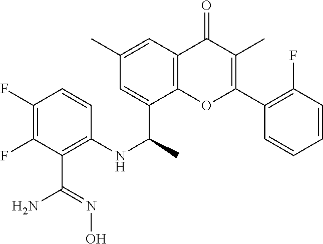 [SMILES: Cc1cc([C@@H](C)Nc2ccc(F)c(F)c2/C(N)=N/O)c2oc(-c3ccccc3F)c(C)c(=O)c2c1] | 482 [M + H]⁺ |
| 258A | 6-[[(1R)-1-[3,6-Dimethyl-4- oxo-2-(3-pyridyl)chromen- 8-yl]ethyl]amino]-2,3- difluoro-N′-hydroxy- benzamidine | 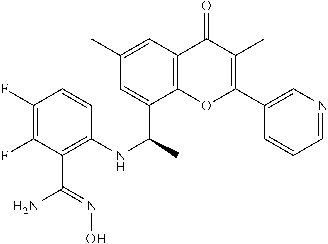 [SMILES: Cc1cc([C@@H](C)Nc2ccc(F)c(F)c2/C(N)=N/O)c2oc(-c3cccnc3)c(C)c(=O)c2c1] | 465 [M + H]⁺ |
| 259A | 2-Fluoro-6-[[(1R)-1-[2-(2- fluorophenyl)-3,6-dimethyl- 4-oxo-chromen-8-yl]- ethyl]amino]-N′-hydroxy- benzamidine | 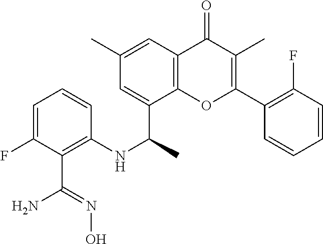 [SMILES: Cc1cc([C@@H](C)Nc2cccc(F)c2/C(N)=N/O)c2oc(-c3ccccc3F)c(C)c(=O)c2c1] | 464 [M + H]⁺ |
| 260A | 2-[[(1R)-1-(3,6-Dimethyl-4- oxo-2-phenyl-chromen-8- yl)ethyl]amino]-5-fluoro-N′- hydroxy-benzamidine | 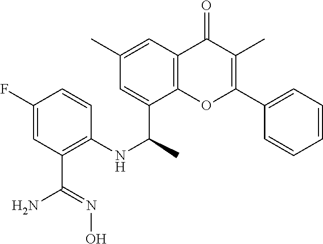 [SMILES: Cc1cc([C@@H](C)Nc2ccc(F)cc2/C(N)=N/O)c2oc(-c3ccccc3)c(C)c(=O)c2c1] | 446 [M + H]⁺ |
| 261A | 3-Chloro-6-[[(1R)-1-(3,6- dimethyl-4-oxo-2-phenyl- chromen-8-yl)ethyl]amino]- 2-fluoro-N′-hydroxy- benzamidine | 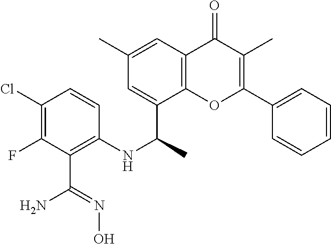 [SMILES: Cc1cc([C@@H](C)Nc2ccc(Cl)c(F)c2/C(N)=N/O)c2oc(-c3ccccc3)c(C)c(=O)c2c1] | 480 [M + H]+ |
| 262A | 6-[[(1R)-1-(3,6-Dimethyl-4- oxo-2-phenyl-chromen-8- yl)ethyl]amino]-2,3-difluoro- N′-hydroxy-benzamidine | 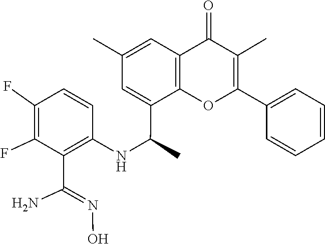 [SMILES: Cc1cc([C@@H](C)Nc2ccc(F)c(F)c2/C(N)=N/O)c2oc(-c3ccccc3)c(C)c(=O)c2c1] | 464 [M + H]⁺ |
| 263A | 3-[[(1R)-1-[2-(5-Fluoro-3- pyridyl)-3,6-dimethyl-4- oxo-chromen-8-yl]ethyl]- amino]-N′-hydroxy-pyridine- 2-carboxamidine | 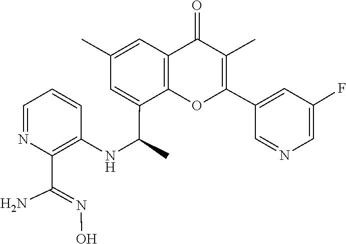 [SMILES: Cc1cc([C@@H](C)Nc2cccnc2/C(N)=N/O)c2oc(-c3cncc(F)c3)c(C)c(=O)c2c1] | 448 [M + H]⁺ |
| 264A | 5-Chloro-2-[(1R)-1-[3,6- dimethyl-4-oxo-2-(3- pyridyl)chromen-8-yl]- ethyl]amino]-N′-hydroxy- benzamidine | 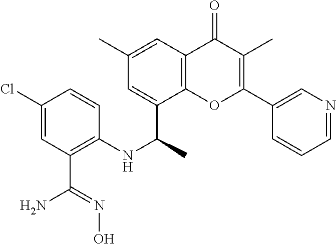 [SMILES: Cc1cc([C@@H](C)Nc2ccc(Cl)cc2/C(N)=N/O)c2oc(-c3cccnc3)c(C)c(=O)c2c1] | 463 [M + H]⁺ |
| 265A | 3-[[(1R)-1-[2-(3,4-Difluoro- phenyl)-3,6-dimethyl-4-oxo- chromen-8-yl]ethyl]amino]- N′-hydroxy-6-methyl- pyridine-2-carboxamidine | 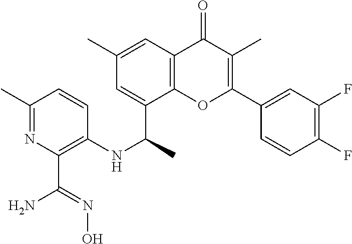 [SMILES: Cc1cc([C@@H](C)Nc2ccc(C)nc2/C(N)=N/O)c2oc(-c3ccc(F)c(F)c3)c(C)c(=O)c2c1] | 479 [M + H]⁺ |
| 266A | 3-[[(1R)-1-[3,6-Dimethyl-2- (1-methylpyrazol-4-yl)-4- oxo-chromen-8-yl]ethyl]- amino]-N′-hydroxy-pyridine- 2-carboxamidine | 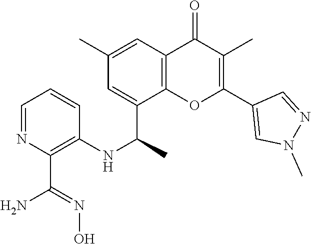 [SMILES: Cc1cc([C@@H](C)Nc2cccnc2/C(N)=N/O)c2oc(-c3cnn(C)c3)c(C)c(=O)c2c1] | 433 [M + H]⁺ |
| 267A | 3-[(1R)-1-[2-(2-Fluoro-3- pyridyl)-3,6-dimethyl-4-oxo- chromen-8-yl]ethyl]amino]- N′-hydroxy-pyridine-2- carboxamidine | 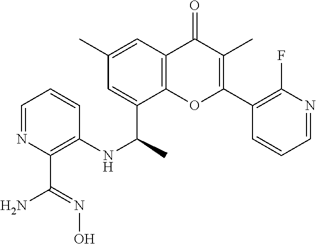 [SMILES: Cc1cc([C@@H](C)Nc2cccnc2/C(N)=N/O)c2oc(-c3cccnc3F)c(C)c(=O)c2c1] | 448 [M + H]⁺ |
| 268A | 3-[[(1R)-1-[2-(2-Fluoro- phenyl)-3,6-dimethyl-4-oxo- chromen-8-yl]ethyl]amino]- N′-hydroxy-pyridine-2- carboxamidine | 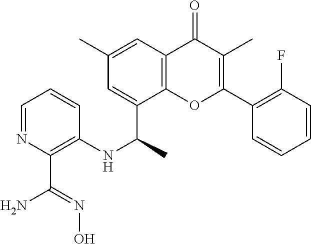 [SMILES: Cc1cc([C@@H](C)Nc2cccnc2/C(N)=N/O)c2oc(-c3ccccc3F)c(C)c(=O)c2c1] | 447 [M + H]⁺ |
| 269A | 2-[(1R)-1-[2-(2,3-Difluoro- phenyl)-3,6-dimethyl-4- oxo-chromen-8-yl]ethyl]- amino]-6-fluoro-N′- hydroxy-benzamidine | 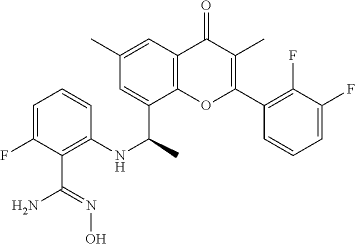 [SMILES: Cc1cc([C@@H](C)Nc2cccc(F)c2/C(N)=N/O)c2oc(-c3cccc(F)c3F)c(C)c(=O)c2c1] | 482 [M + H]⁺ |
| 270A | 3-[[(1R)-1-[3,6-Dimethyl-2- (2-methylindazol-5-yl)-4- oxo-chromen-8-yl]ethyl]- amino]-N′-hydroxy-pyridine- 2-carboxamidine | 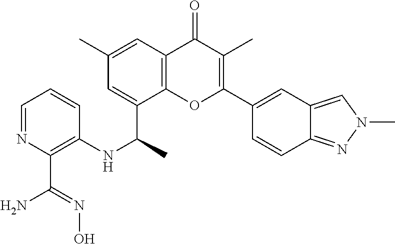 [SMILES: Cc1cc([C@@H](C)Nc2cccnc2/C(N)=N/O)c2oc(-c3ccc4nn(C)cc4c3)c(C)c(=O)c2c1] | 483 [M + H]⁺ |
| 271A | 2-[[(1R)-1-[2-(2,4-Difluoro- phenyl)-3,6-dimethyl-4- oxo-chromen-8-yl]ethyl]- amino]-6-fluoro-N′- hydroxy-benzamidine | 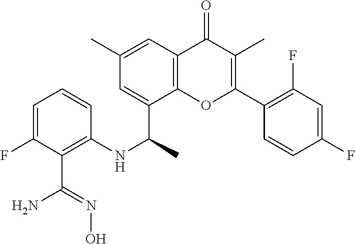 [SMILES: Cc1cc([C@@H](C)Nc2cccc(F)c2/C(N)=N/O)c2oc(-c3ccc(F)cc3F)c(C)c(=O)c2c1] | 482 [M + H]⁺ |
| 272A | 3-[[(1R)-1-[2-[6-(Difluoro- methyl)-2-pyridyl]-3,6- dimethyl-4-oxo-chromen-8- yl]ethyl]amino]-N′-hydroxy- pyridine-2-carboxamidine | 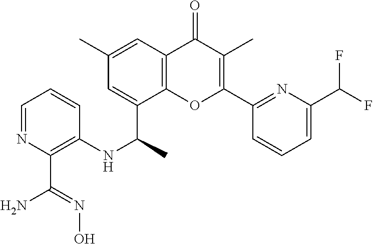 [SMILES: Cc1cc([C@@H](C)Nc2cccnc2/C(N)=N/O)c2oc(-c3cccc(C(F)F)n3)c(C)c(=O)c2c1] | 480 [M + H]⁺ |
| 273A | 2-Chloro-6-[[(1R)-1-(3,6- dimethyl-4-oxo-2-phenyl- chromen-8-yl)ethyl]amino]- N′-hydroxy-benzamidine | 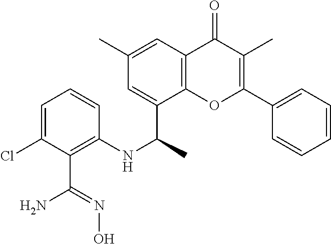 [SMILES: Cc1cc([C@@H](C)Nc2cccc(Cl)c2/C(N)=N/O)c2oc(-c3ccccc3)c(C)c(=O)c2c1] | 462 [M + H]⁺ |
| 274A | 3-[[(1R)-1-[2-[6-(Difluoro- methyl)-2-pyridyl]-3,6- dimethyl-4-oxo-chromen-8- yl]ethyl]amino]-N′-hydroxy- 6-methyl-pyridine-2-carbox- amidine | 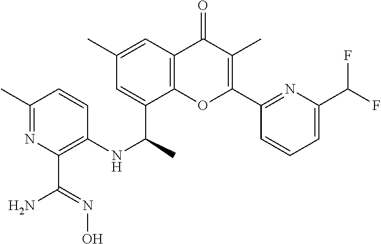 [SMILES: Cc1cc([C@@H](C)Nc2ccc(C)nc2/C(N)=N/O)c2oc(-c3cccc(C(F)F)n3)c(C)c(=O)c2c1] | 494 [M + H]⁺ |
| 275A | 6-Chloro-3-[[(1R)-1-[3,6- dimethyl-2-(1-methylpyrazol- 4-yl)-4-oxo-chromen-8-yl]- ethyl]amino]-N′-hydroxy- pyridine-2-carboxamidine | 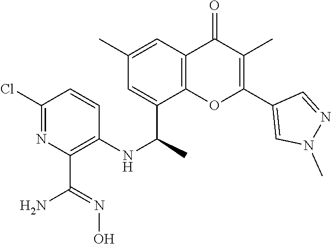 [SMILES: Cc1cc([C@@H](C)Nc2ccc(Cl)nc2/C(N)=N/O)c2oc(-c3cnn(C)c3)c(C)c(=O)c2c1] | 467 [M + H]⁺ |
| 276A | 6-Chloro-3-[(1R)-1-[2-(6- fluoro-2-pyridyl)-3,6- dimethyl-4-oxo-chromen-8- yl]ethyl]amino]-N′-hydroxy- pyridine-2-carboxamidine | 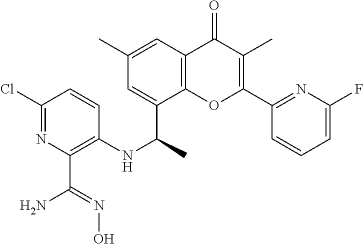 [SMILES: Cc1cc([C@@H](C)Nc2ccc(Cl)nc2/C(N)=N/O)c2oc(-c3cccc(F)n3)c(C)c(=O)c2c1] | 482 [M + H]⁺ |
| 277A | 6-Chloro-3-[[(1R)-1-[3,6- dimethyl-4-oxo-2-(3- pyridyl)chromen-8-yl]- ethyl]amino]-N′-hydroxy- pyridine-2-carboxamidine | 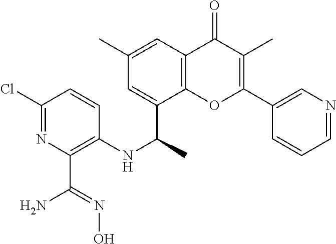 [SMILES: Cc1cc([C@@H](C)Nc2ccc(Cl)nc2/C(N)=N/O)c2oc(-c3cccnc3)c(C)c(=O)c2c1] | 464 [M + H]⁺ |
| 278A | 6-Chloro-3-[(1R)-1-[2-(2- fluoro-3-pyridyl)-3,6- dimethyl-4-oxo-chromen-8- yl]ethyl]amino]-N′-hydroxy- pyridine-2-carboxamidine | 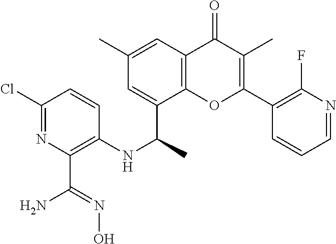 [SMILES: Cc1cc([C@@H](C)Nc2ccc(Cl)nc2/C(N)=N/O)c2oc(-c3cccnc3F)c(C)c(=O)c2c1] | 482 [M + H]⁺ |
| 279A | 6-Chloro-3-[(1R)-1-[2-(3- fluoro-2-pyridyl)-3,6- dimethyl-4-oxo-chromen-8- yl]ethyl]amino]-N′-hydroxy- pyridine-2-carboxamidine | 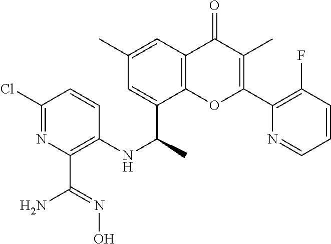 [SMILES: Cc1cc([C@@H](C)Nc2ccc(Cl)nc2/C(N)=N/O)c2oc(-c3ncccc3F)c(C)c(=O)c2c1] | 482 [M + H]⁺ |
| 280A | 6-Chloro-3-[(1R)-1-[2-(2,5- difluoro-3-pyridyl)-3,6- dimethyl-4-oxo-chromen-8- ylethyl]amino]-N′-hydroxy- pyridine-2-carboxamidine | 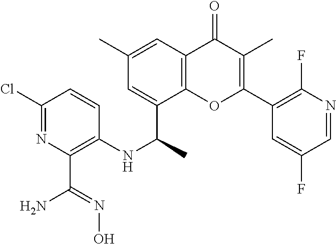 [SMILES: Cc1cc([C@@H](C)Nc2ccc(Cl)nc2/C(N)=N/O)c2oc(-c3cc(F)cnc3F)c(C)c(=O)c2c1] | 500 [M + H]⁺ |
| 281A | 6-Chloro-N′-hydroxy-3- [(1R)-1-[2-(6-methoxy-2- pyridyl)-3,6-dimethyl-4-oxo- chromen-8-yl]ethyl]amino]- pyridine-2-carboxamidine | 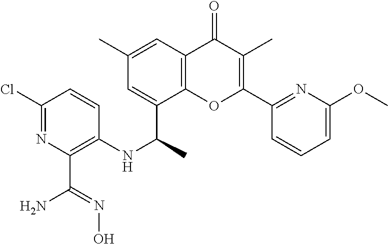 [SMILES: COc1cccc(-c2oc3c([C@@H](C)Nc4ccc(Cl)nc4/C(N)=N/O)cc(C)cc3c(=O)c2C)n1] | 494 [M + H]⁺ |
| 282A | 6-Chloro-N′-hydroxy-3- [(1R)-1-[3-methyl-4-oxo- 2-(2-pyridyl)-6-(trifluoro- methyl)chromen-8-yl]ethyl]- amino]pyridine-2-carbox- amidine | 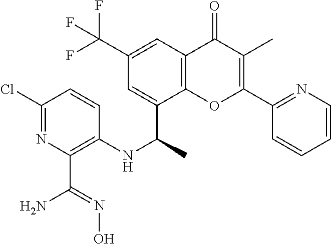 [SMILES: Cc1c(-c2ccccn2)oc2c([C@@H](C)Nc3ccc(Cl)nc3/C(N)=N/O)cc(C(F)(F)F)cc2c1=O] | 518 [M + H]⁺ |
| 283A | 6-Chloro-N′-hydroxy-3- [(1R)-1-[6-methyl-2-(1- methylpyrazol-4-yl)-4-oxo- chromen-8-yl]ethyl]amino]- pyridine-2-carboxamidine | 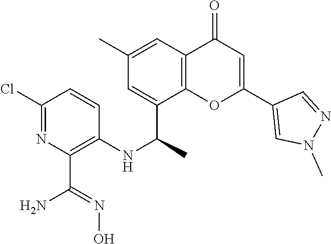 [SMILES: Cc1cc([C@@H](C)Nc2ccc(Cl)nc2/C(N)=N/O)c2oc(-c3cnn(C)c3)cc(=O)c2c1] | 453 [M + H]⁺ |
| 284A | 3-[[(1R)-1-[2-(5-Fluoro-3- pyridyl)-3-methyl-4-oxo-6- (trifluoromethyl)chromen-8- yl]ethyl]amino]-N′-hydroxy- pyridine-2-carboxamidine | 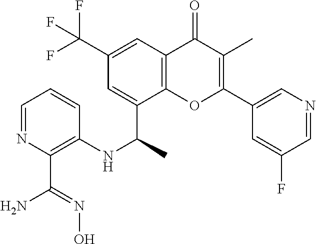 [SMILES: Cc1c(-c2cncc(F)c2)oc2c([C@@H](C)Nc3cccnc3/C(N)=N/O)cc(C(F)(F)F)cc2c1=O] | 502 [M + H]⁺ |
| 285A | N′-Hydroxy-3-[(1R)-1-[3- methyl-4-oxo-2-(2-pyridyl)- 6-(trifluoromethyl)chromen- 8-yl]ethyl]amino]pyridine- 2-carboxamidine | 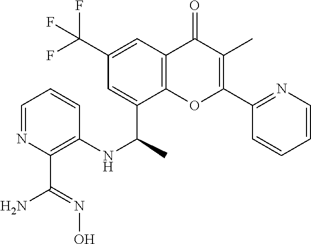 [SMILES: Cc1c(-c2ccccn2)oc2c([C@@H](C)Nc3cccnc3/C(N)=N/O)cc(C(F)(F)F)cc2c1=O] | 484 [M + H]⁺ |
| 286A | 6-[[(1R)-1-(3,6-Dimethyl- 4-oxo-2-pyrimidin-5-yl- chromen-8-yl)ethyl]amino]- 2,3-difluoro-N′-hydroxy- benzamidine | 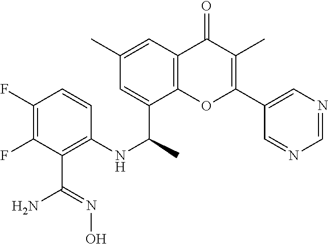 [SMILES: Cc1cc([C@@H](C)Nc2ccc(F)c(F)c2/C(N)=N/O)c2oc(-c3cncnc3)c(C)c(=O)c2c1] | 466 [M + H]⁺ |
| 319C | 6-Chloro-3-[[(1R)-1-[3,6- dimethyl-4-oxo-2-(1H- pyrazol-4-yl)chromen-8- yl]ethyl]amino]-N′-hydroxy- pyridine-2-carboxamidine | 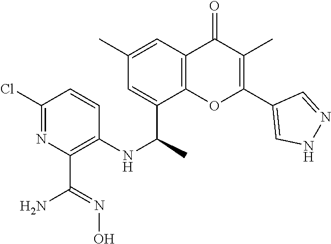 [SMILES: Cc1cc([C@@H](C)Nc2ccc(Cl)nc2/C(N)=N/O)c2oc(-c3cn[nH]c3)c(C)c(=O)c2c1] | 453 [M + H]⁺ |
| 320C | 6-Chloro-3-[(1R)-1-[2-(2,5- dimethylpyrazol-3-yl)-3,6- dimethyl-4-oxo-chromen-8- yl]ethyl]amino]-N′-hydroxy- pyridine-2-carboxamidine | 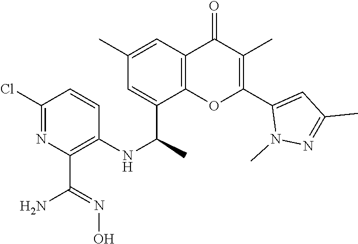 [SMILES: Cc1cc([C@@H](C)Nc2ccc(Cl)nc2/C(N)=N/O)c2oc(-c3cc(C)nn3C)c(C)c(=O)c2c1] | 481 [M + H]⁺ |
| 321C | 3-[[(1R)-1-[2-[1-[2-[tert- Butyl(dimethyl)silyl]oxy- ethyl]pyrazol-4-yl]-3,6- dimethyl-4-oxo-chromen- 8-yl]ethyl]amino]-6-chloro- N′-hydroxy-pyridine-2- carboxamidine | 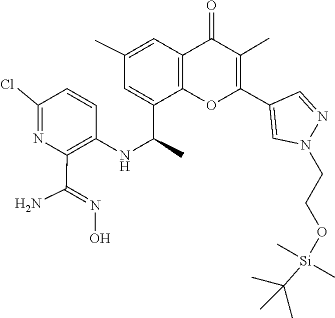 [SMILES: Cc1cc([C@@H](C)Nc2ccc(Cl)nc2/C(N)=N/O)c2oc(-c3cnn(CCO[Si](C)(C)C(C)(C)C)c3)c(C)c(=O)c2c1] | 611/613 [M + H]⁺ |
| 322C | 6-Chloro-3-[[(1R)-1-[3,6- dimethyl-4-oxo-2-[1- (trifluoromethyl)pyrazol- 4-yl]chromen-8-yl]ethyl]- amino]-N′-hydroxy-pyridine- 2-carboxamidine | 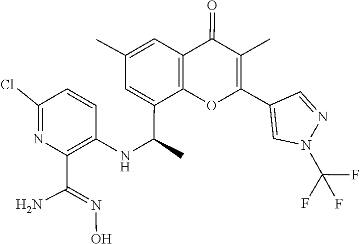 [SMILES: Cc1cc([C@@H](C)Nc2ccc(Cl)nc2/C(N)=N/O)c2oc(-c3cnn(C(F)(F)F)c3)c(C)c(=O)c2c1] | 521 [M + H]⁺ |
| 323C | 6-Chloro-3-[(1R)-1-(3,6- dimethyl-4-oxo-2-pyrazin- 2-yl-chromen-8-yl)ethyl]- amino]-N′-hydroxy-pyridine- 2-carboxamidine | 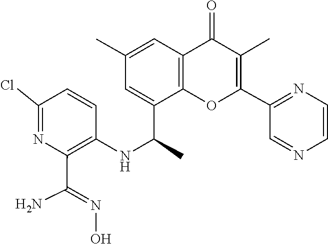 [SMILES: Cc1cc([C@@H](C)Nc2ccc(Cl)nc2/C(N)=N/O)c2oc(-c3cnccn3)c(C)c(=O)c2c1] | 465 [M + H]⁺ |
| 324C | 6-Chloro-3-[[(1R)-1-[3,6- dimethyl-2-(2-methyltriazol- 4-yl)-4-oxo-chromen-8-yl]- ethyl]amino]-N′-hydroxy- pyridine-2-carboxamidine | 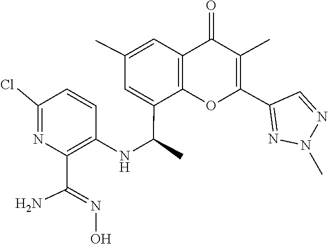 [SMILES: Cc1cc([C@@H](C)Nc2ccc(Cl)nc2/C(N)=N/O)c2oc(-c3cnn(C)n3)c(C)c(=O)c2c1] | 468 [M + H]⁺ |
| 325C | 6-Chloro-3-[[(1R)-1-[2-(3- fluoro-1-methyl-pyrazol-4- yl)-3,6-dimethyl-4-oxo- chromen-8-yl]ethyl]amino]- N′-hydroxy-pyridine-2- carboxamidine | 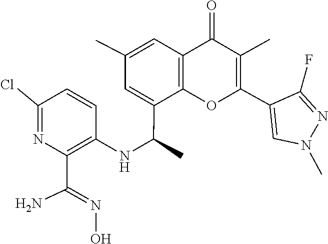 [SMILES: Cc1cc([C@@H](C)Nc2ccc(Cl)nc2/C(N)=N/O)c2oc(-c3cn(C)nc3F)c(C)c(=O)c2c1] | 485 [M + H]⁺ |
| 326C | 6-Chloro-N′-hydroxy-3- [(1R)-1-(6-methyl-3-oxazol- 4-yl-4-oxo-2-phenyl-chro- men-8-yl)ethyl]amino]- pyridine-2-carboxamidine | 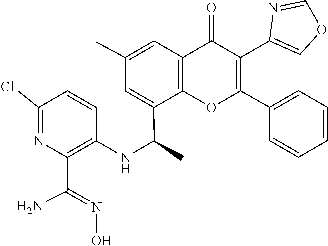 [SMILES: Cc1cc([C@@H](C)Nc2ccc(Cl)nc2/C(N)=N/O)c2oc(-c3ccccc3)c(-c3cocn3)c(=O)c2c1] | 516 [M + H]⁺ |
| 327C | 6-Chloro-N′-hydroxy-3- [(1R)-1-[2-[1-(2-methoxy- ethyl)pyrazol-4-yl]-3,6- dimethyl-4-oxo-chromen-8- ylethyl]amino]pyridine-2- carboxamidine | 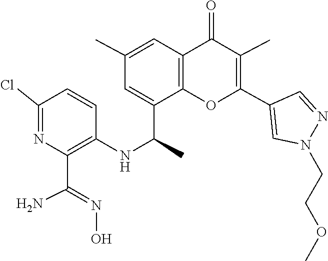 [SMILES: COCCn1cc(-c2oc3c([C@@H](C)Nc4ccc(Cl)nc4/C(N)=N/O)cc(C)cc3c(=O)c2C)cn1] | 511 [M + H]⁺ |
| 328C | 6-Chloro-N′-hydroxy-3- [(1R)-1-[2-[1-(2-hydroxy-2- methyl-propyl)pyrazol-4- yl]-3,6-dimethyl-4-oxo- chromen-8-yl]ethyl]amino]- pyridine-2-carboxamidine | 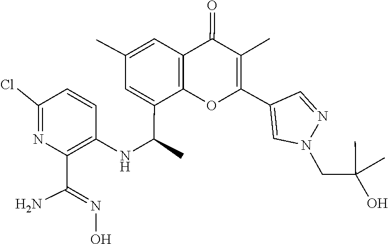 [SMILES: Cc1cc([C@@H](C)Nc2ccc(Cl)nc2/C(N)=N/O)c2oc(-c3cnn(CC(C)(C)O)c3)c(C)c(=O)c2c1] | 525 [M + H]⁺ |
| 329C | 6-Chloro-3-[(1R)-1-[3,6- dimethyl-4-oxo-2-(1-tetra- hydropyran-4-ylpyrazol-4- yl)chromen-8-yl]ethyl]- amino]-N′-hydroxy-pyridine- 2-carboxamidine | 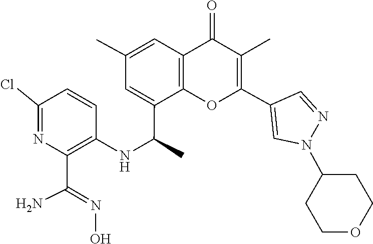 [SMILES: Cc1cc([C@@H](C)Nc2ccc(Cl)nc2/C(N)=N/O)c2oc(-c3cnn(C4CCOCC4)c3)c(C)c(=O)c2c1] | 537 [M + H]⁺ |
| 330C | 6-Chloro-3-[(1R)-1-[2-(1,5- dimethylpyrazol-4-yl)-3,6- dimethyl-4-oxo-chromen-8- yl]ethyl]amino]-N′-hydroxy- pyridine-2-carboxamidine | 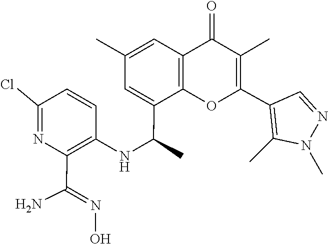 [SMILES: Cc1cc([C@@H](C)Nc2ccc(Cl)nc2/C(N)=N/O)c2oc(-c3cnn(C)c3C)c(C)c(=O)c2c1] | 481 [M + H]⁺ |
| 331C | 6-Chloro-3-[(1R)-1-[2-[1- (1-cyano-1-methyl-ethyl)- pyrazol-4-yl]-3,6-dimethyl- 4-oxo-chromen-8-yl]ethyl]- amino]-N′-hydroxy-pyridine- 2-carboxamidine | 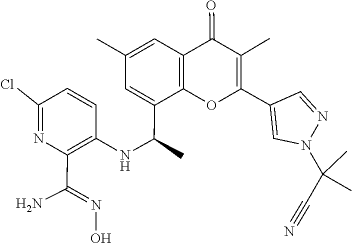 [SMILES: Cc1cc([C@@H](C)Nc2ccc(Cl)nc2/C(N)=N/O)c2oc(-c3cnn(C(C)(C)C#N)c3)c(C)c(=O)c2c1] | 520 [M + H]⁺ |
| 332C | 6-Chloro-N′-hydroxy-3- [(1R)-1-[6-methyl-2-(1- methylpyrazol-4-yl)-3- oxazol-4-yl-4-oxo-chromen- 8-yl]ethyl]amino]pyridine- 2-carboxamidine | 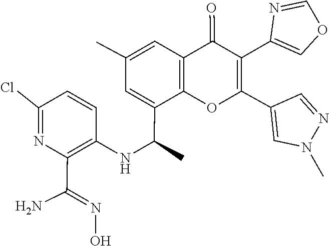 [SMILES: Cc1cc([C@@H](C)Nc2ccc(Cl)nc2/C(N)=N/O)c2oc(-c3cnn(C)c3)c(-c3cocn3)c(=O)c2c1] | 520 [M + H]⁺ |
| 333C | 6-Chloro-3-[[(1R)-1-[3,6- dimethyl-2-(1-methyl- pyrazolo-[4,3-b]pyridin- 5-yl)-4-oxo-chromen-8- yl]ethyl]amino]-N′-hydroxy- pyridine-2-carboxamidine | 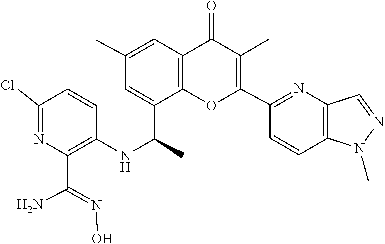 [SMILES: Cc1cc([C@@H](C)Nc2ccc(Cl)nc2/C(N)=N/O)c2oc(-c3ccc4c(cnn4C)n3)c(C)c(=O)c2c1] | 518 [M + H]⁺ |
| 334C | 6-Chloro-3-[(1R)-1-[2-(3,6- difluoro-2-pyridyl)-3,6- dimethyl-4-oxo-chromen-8- yl]ethyl]amino]-N′-hydroxy- pyridine-2-carboxamidine | 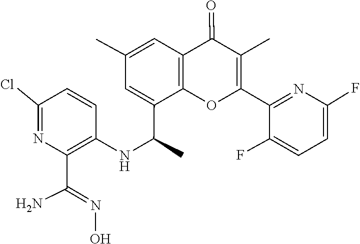 [SMILES: Cc1cc([C@@H](C)Nc2ccc(Cl)nc2/C(N)=N/O)c2oc(-c3nc(F)ccc3F)c(C)c(=O)c2c1] | 500 [M + H]⁺ |
| 335C | 6-Chloro-3-[[(1R)-1-[2-(2,4- difluoro-3-pyridyl)-3,6- dimethyl-4-oxo-chromen-8- yl]ethyl]amino]-N′-hydroxy- pyridine-2-carboxamidine | 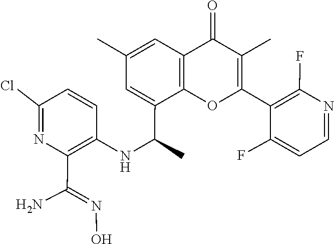 [SMILES: Cc1cc([C@@H](C)Nc2ccc(Cl)nc2/C(N)=N/O)c2oc(-c3c(F)ccnc3F)c(C)c(=O)c2c1] | 500 [M + H]⁺ |
| 336C | 6-Chloro-3-[(1R)-1-[3,6- dimethyl-2-(2-methylpyr- azol-3-yl)-4-oxo-chromen-8- yl]ethyl]amino]-N′-hydroxy- pyridine-2-carboxamidine | 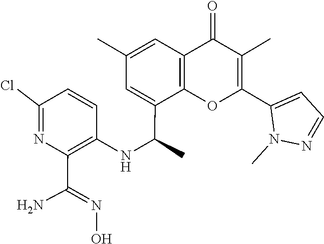 [SMILES: Cc1cc([C@@H](C)Nc2ccc(Cl)nc2/C(N)=N/O)c2oc(-c3ccnn3C)c(C)c(=O)c2c1] | 467 [M + H]⁺ |
| 337C | 6-Chloro-3-[(1R)-1-[2-[1- (difluoromethyl)pyrazol-4- yl]-3,6-dimethyl-4-oxo- chromen-8-yl]ethyl]amino]- N′-hydroxy-pyridine-2- carboxamidine | 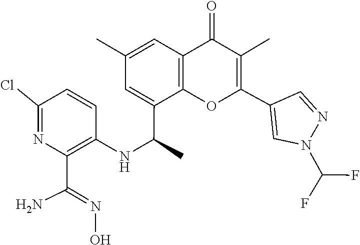 [SMILES: Cc1cc([C@@H](C)Nc2ccc(Cl)nc2/C(N)=N/O)c2oc(-c3cnn(C(F)F)c3)c(C)c(=O)c2c1] | 503 [M + H]⁺ |
| 338C | 6-Chloro-N′-hydroxy-3- [(1R)-1-[2-(1-isopropyl- pyrazol-4-yl)-3,6-dimethyl- 4-oxo-chromen-8-yl]ethyl]- amino]pyridine-2-carbox- amidine | 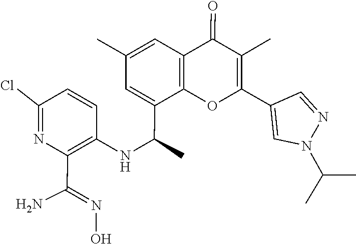 [SMILES: Cc1cc([C@@H](C)Nc2ccc(Cl)nc2/C(N)=N/O)c2oc(-c3cnn(C(C)C)c3)c(C)c(=O)c2c1] | 495 [M + H]⁺ |
| 339C | 6-Chloro-3-[[(1R)-1-[2-(1- cyclopropylpyrazol-4-yl)- 3,6-dimethyl-4-oxo- chromen-8-yl]ethyl]amino]- N′-hydroxy-pyridine-2- carboxamidine | 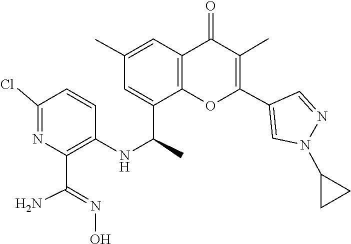 [SMILES: Cc1cc([C@@H](C)Nc2ccc(Cl)nc2/C(N)=N/O)c2oc(-c3cnn(C4CC4)c3)c(C)c(=O)c2c1] | 493 [M + H]⁺ |
| 340C | 6-Chloro-3-[(1R)-1-(3,6- dimethyl-4-oxo-2- pyrimidin-5-yl-chromen-8- yl)ethyl]amino]-N′-hydroxy- pyridine-2-carboxamidine | 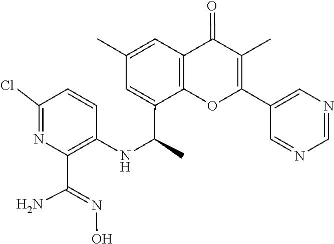 [SMILES: Cc1cc([C@@H](C)Nc2ccc(Cl)nc2/C(N)=N/O)c2oc(-c3cncnc3)c(C)c(=O)c2c1] | 465 [M + H]⁺ |
| 341C | 6-Chloro-3-[[(1R)-1-[2-(4- fluoro-3-pyridyl)-3,6- dimethyl-4-oxo-chromen-8- ylethyl]amino]-N′-hydroxy- pyridine-2-carboxamidine | 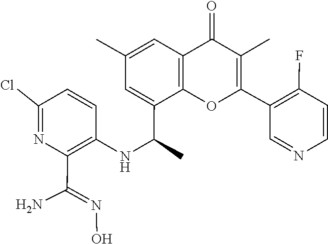 [SMILES: Cc1cc([C@@H](C)Nc2ccc(Cl)nc2/C(N)=N/O)c2oc(-c3cnccc3F)c(C)c(=O)c2c1] | 482 [M + H]⁺ |
| 342C | 6-Chloro-3-[(1R)-1-[2-(5- fluoro-2-pyridyl)-3,6- dimethyl-4-oxo-chromen-8- yl]ethyl]amino]-N′-hydroxy- pyridine-2-carboxamidine | 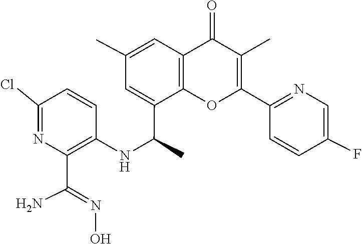 [SMILES: Cc1cc([C@@H](C)Nc2ccc(Cl)nc2/C(N)=N/O)c2oc(-c3ccc(F)cn3)c(C)c(=O)c2c1] | 482 [M + H]⁺ |
| 343C | 6-Chloro-3-[[(1R)-1-[3,6- dimethyl-4-oxo-2-[1- (trideuteriomethyl)pyrazol- 4-yl]chromen-8-yl]ethyl]- amino]-N′-hydroxy-pyr- idine-2-carboxamidine | 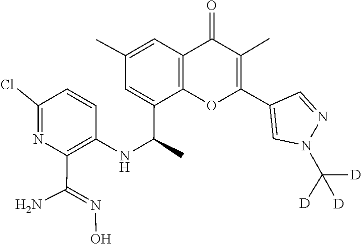 [SMILES: [2H]C([2H])([2H])n1cc(-c2oc3c([C@@H](C)Nc4ccc(Cl)nc4/C(N)=N/O)cc(C)cc3c(=O)c2C)cn1] | 470 [M + H]⁺ |
| 344C | 6-Chloro-3-[(1R)-1-(3,6- dimethyl-4-oxo-2-pyrimidin- 2-yl-chromen-8-yl)ethyl]- amino]-N′-hydroxy-pyridine- 2-carboxamidine | 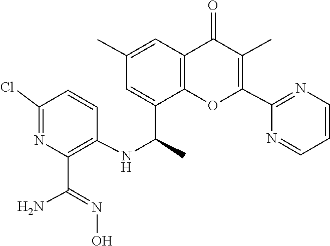 [SMILES: Cc1cc([C@@H](C)Nc2ccc(Cl)nc2/C(N)=N/O)c2oc(-c3ncccn3)c(C)c(=O)c2c1] | 465 [M + H]⁺ |
| 345C | 6-Chloro-3-[(1R)-1-[3,6- dimethyl-2-(1-methylindazol- 3-yl)-4-oxo-chromen-8-yl]- ethyl]amino]-N'-hydroxy- pyridine-2-carboxamidine | 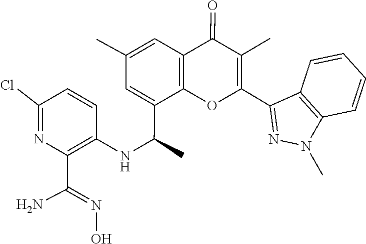 [SMILES: Cc1cc([C@@H](C)Nc2ccc(Cl)nc2/C(N)=N/O)c2oc(-c3nn(C)c4ccccc34)c(C)c(=O)c2c1] | 517 [M + H]⁺ |
| 346C | 6-Chloro-N′-hydroxy-3- [(1R)-1-[2-(6-methoxy-2- pyridyl)-3,6-dimethyl-4-oxo- chromen-8-yl]ethyl]amino]- pyridine-2-carboxamidine | 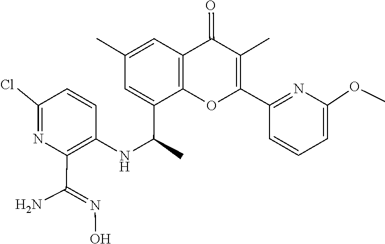 [SMILES: COc1cccc(-c2oc3c([C@@H](C)Nc4ccc(Cl)nc4/C(N)=N/O)cc(C)cc3c(=O)c2C)n1] | 494 [M + H]⁺ |
| 347C¹ | 6-Chloro-3-[(1R)-1-[3,6- dimethyl-2-(2-methyl- pyrazolo-[4,3-b]pyridin-5- yl)-4-oxo-chromen-8-yl]- ethyl]amino]-N'-hydroxy- pyridine-2-carboxamidine | 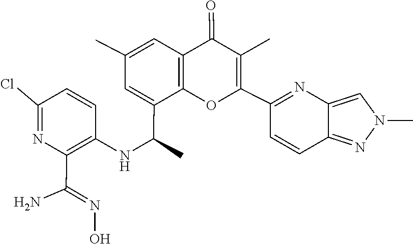 [SMILES: Cc1cc([C@@H](C)Nc2ccc(Cl)nc2/C(N)=N/O)c2oc(-c3ccc4nn(C)cc4n3)c(C)c(=O)c2c1] | 518 [M + H]⁺ |
| 348C | 6-Chloro-N′-hydroxy-3- [(1R)-1-[3-iodo-6-methyl-4- oxo-2-(2-pyridyl)chromen-8- yl]ethyl]amino]pyridine-2- carboxamidine | 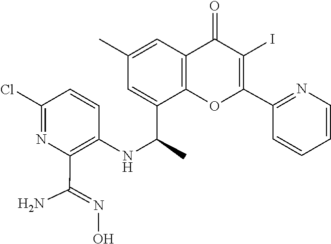 [SMILES: Cc1cc([C@@H](C)Nc2ccc(Cl)nc2/C(N)=N/O)c2oc(-c3ccccn3)c(I)c(=O)c2c1] | 576 [M + H]⁺ |
| 349C | 6-Chloro-3-[(1R)-1-deuterio- 1-(3,6-dimethyl-4-oxo-2- phenyl-chromen-8-yl)ethyl]- amino]-N′-hydroxy-pyridine- 2-carboxamidine | 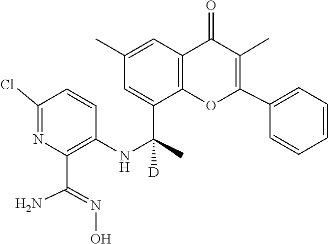 [SMILES: [2H][C@](C)(Nc1ccc(Cl)nc1/C(N)=N/O)c1cc(C)cc2c(=O)c(C)c(-c3ccccc3)oc12] | 464 [M + H]⁺ |
| 350C | 3-[[(1R)-1-Deuterio-1-(3,6- dimethyl-4-oxo-2-phenyl- chromen-8-yl)ethyl]amino]- N′-hydroxy-pyridine-2- carboxamidine | 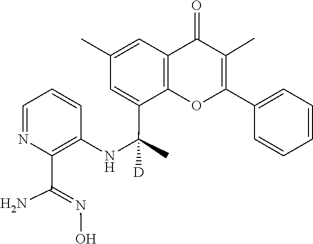 [SMILES: [2H][C@](C)(Nc1cccnc1/C(N)=N/O)c1cc(C)cc2c(=O)c(C)c(-c3ccccc3)oc12] | 430 [M + H]⁺ |
| 351C | 6-Chloro-3-[(1R)-1-[3,6- dimethyl-2-(1-methylind- azol-5-yl)-4-oxo-chromen- 8-yl]ethyl]amino]-N′- hydroxy-pyridine-2- carboxamidine | 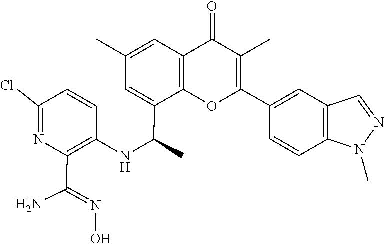 [SMILES: Cc1cc([C@@H](C)Nc2ccc(Cl)nc2/C(N)=N/O)c2oc(-c3ccc4c(cnn4C)c3)c(C)c(=O)c2c1] | 517 [M + H]⁺ |
| 352C | 2-Fluoro-6-[(1R)-1-[2-(5- fluoro-3-pyridyl)-3,6- dimethyl-4-oxo-chromen-8- yl]ethyl]amino]-N′-hydroxy- benzamidine | 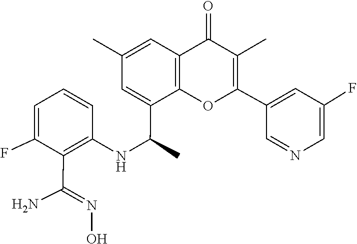 [SMILES: Cc1cc([C@@H](C)Nc2cccc(F)c2/C(N)=N/O)c2oc(-c3cncc(F)c3)c(C)c(=O)c2c1] | 465 [M + H]⁺ |
| 353C | 6-[[(1R)-1-[3,6-Dimethyl-4- oxo-2-(2-pyridyl)chromen-8- yl]ethyl]amino]-2,3-difluoro- N′-hydroxy-benzamidine | 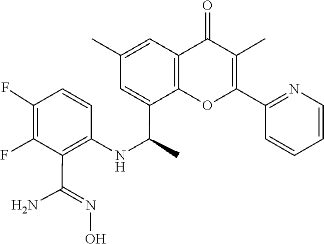 [SMILES: Cc1cc([C@@H](C)Nc2ccc(F)c(F)c2/C(N)=N/O)c2oc(-c3ccccn3)c(C)c(=O)c2c1] | 465 [MH]⁺ |
| 354C | 6-[[(1R)-1-[3,6-Dimethyl-2- (1-methylpyrazol-4-yl)-4- oxo-chromen-8-yl]ethyl]- amino]-2,3-difluoro-N′- hydroxy-benzamidine | 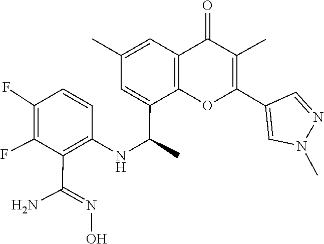 [SMILES: Cc1cc([C@@H](C)Nc2ccc(F)c(F)c2/C(N)=N/O)c2oc(-c3cnn(C)c3)c(C)c(=O)c2c1] | 468 [M + H]⁺ |
| 355C | 6-Chloro-3-[[(1R)-1-[2-(2,6- difluorophenyl)-3,6-dimeth- yl-4-oxo-chromen-8-yl]- ethyl]amino]-N′-hydroxy- pyridine-2-carboxamidine | 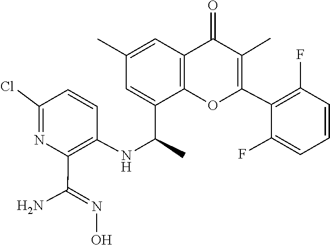 [SMILES: Cc1cc([C@@H](C)Nc2ccc(Cl)nc2/C(N)=N/O)c2oc(-c3c(F)cccc3F)c(C)c(=O)c2c1] | 499 [M + H]⁺ |
| 356C | 6-[[(1R)-1-[3,6-Dimethyl-2- (2-methylindazol-5-yl)-4- oxo-chromen-8-yl]ethyl]- amino]-2,3-difluoro-N′- hydroxy-benzamidine | 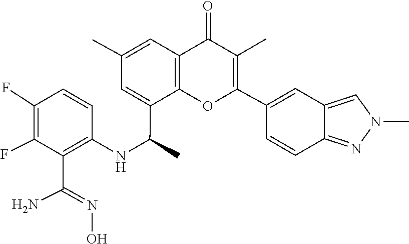 [SMILES: Cc1cc([C@@H](C)Nc2ccc(F)c(F)c2/C(N)=N/O)c2oc(-c3ccc4nn(C)cc4c3)c(C)c(=O)c2c1] | 518 [M + H]⁺ |
| 357C | 6-Chloro-3-[[(1R)-1-[3,6- dimethyl-2-(2-methyl- pyrazolo[3,4-b]pyridin-5- yl)-4-oxo-chromen-8-yl]- ethyl]amino]-N′-hydroxy- pyridine-2-carboxamidine | 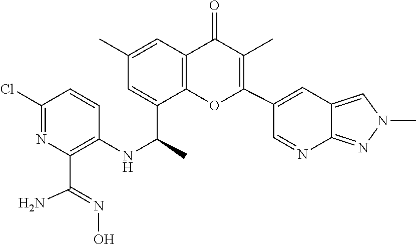 [SMILES: Cc1cc([C@@H](C)Nc2ccc(Cl)nc2/C(N)=N/O)c2oc(-c3cnc4nn(C)cc4c3)c(C)c(=O)c2c1] | 518 [M + H]⁺ |
| 358C | 6-Chloro-N′-hydroxy-3- [[(1R)-1-[3-isoxazol-4-yl-6- methyl-4-oxo-2-(3-pyridyl)- chromen-8-yl]ethyl]amino]- pyridine-2-carboxamidine | 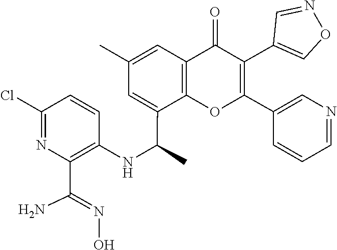 [SMILES: Cc1cc([C@@H](C)Nc2ccc(Cl)nc2/C(N)=N/O)c2oc(-c3cccnc3)c(-c3cnoc3)c(=O)c2c1] | 517 [M + H]⁺ |
| 359C | 6-Chloro-3-[(1R)-1-[2-(4- fluoro-2-pyridyl)-3,6- dimethyl-4-oxo-chromen-8- yl]ethyl]amino]-N′-hydroxy- pyridine-2-carboxamidine | 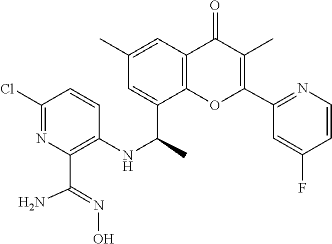 [SMILES: Cc1cc([C@@H](C)Nc2ccc(Cl)nc2/C(N)=N/O)c2oc(-c3cc(F)ccn3)c(C)c(=O)c2c1] | 482 [M + H]⁺ |
| 360C | 6-Chloro-3-[[(1R)-1-[2-(6,7- dihydro-4H-pyrazolo[5,1-c]- [1,4]oxazin-3-yl)-3,6- dimethyl-4-oxo-chromen-8- yl]ethyl]amino]-N′-hydroxy- pyridine-2-carboxamidine | 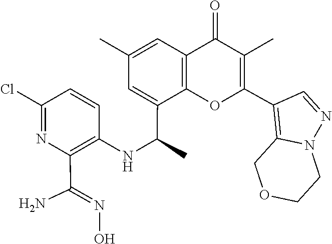 [SMILES: Cc1cc([C@@H](C)Nc2ccc(Cl)nc2/C(N)=N/O)c2oc(-c3cnn4c3COCC4)c(C)c(=O)c2c1] | 509 [M + H]⁺ |
| 361C | 6-Chloro-3-[(1R)-1-[3,6- dimethyl-2-[1-(oxetan-3- yl)pyrazol-4-yl]-4-oxo- chromen-8-yl]ethyl]amino]- N′-hydroxy-pyridine-2- carboxamidine | 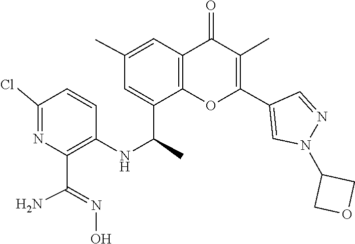 [SMILES: Cc1cc([C@@H](C)Nc2ccc(Cl)nc2/C(N)=N/O)c2oc(-c3cnn(C4COC4)c3)c(C)c(=O)c2c1] | 509 [M + H]⁺ |
| 362C | 6-Chloro-3-[[(1R)-1-[3,6- dimethyl-2-[1-[(3-methyl- oxetan-3-yl)methyl]pyrazol- 4-yl]-4-oxo-chromen-8-yl]- ethyl]amino]-N′-hydroxy- pyridine-2-carboxamidine | 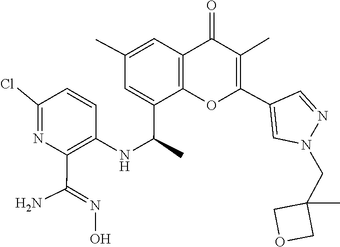 [SMILES: Cc1cc([C@@H](C)Nc2ccc(Cl)nc2/C(N)=N/O)c2oc(-c3cnn(CC4(C)COC4)c3)c(C)c(=O)c2c1] | 537 [M + H]⁺ |
| 363C | tert-Butyl 3-[4-[8-[(1R)-1- [[6-chloro-2-[(Z)-N′- hydroxycarbamimidoyl]-3- pyridyl]amino]ethyl]-3,6- dimethyl-4-oxo-chromen-2- yl]pyrazol-1-yl]azetidine-1- carboxylate | 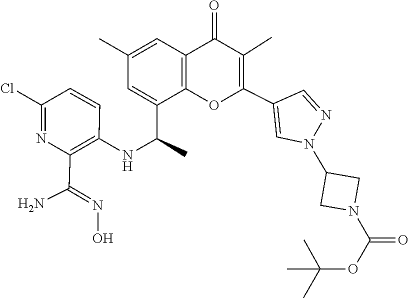 [SMILES: Cc1cc([C@@H](C)Nc2ccc(Cl)nc2/C(N)=N/O)c2oc(-c3cnn(C4CN(C(=O)OC(C)(C)C)C4)c3)c(C)c(=O)c2c1] | 608 [M + H]⁺ |
| 364C | 6-Chloro-3-[(1R)-1-[2-[1- [(2,2-dimethyl-1,3-dioxolan- 4-yl)methyl]pyrazol-4-yl]- 3,6-dimethyl-4-oxo-chro- men-8-yl]ethyl]amino]-N′- hydroxy-pyridine-2-carbox- amidine | 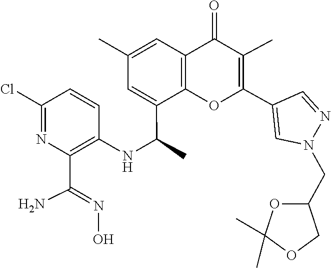 [SMILES: Cc1cc([C@@H](C)Nc2ccc(Cl)nc2/C(N)=N/O)c2oc(-c3cnn(CC4COC(C)(C)O4)c3)c(C)c(=O)c2c1] | 567 [M + H]⁺ |
| 365C | 6-Chloro-3-[[(1R)-1-(3,6- dimethyl-4-oxo-2-pyr- azolo[1,5-a]pyrimidin-3-yl- chromen-8-yl)ethyl]amino]- N′-hydroxy-pyridine-2- carboxamidine | 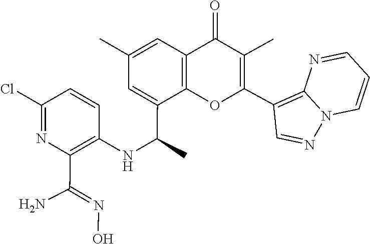 [SMILES: Cc1cc([C@@H](C)Nc2ccc(Cl)nc2/C(N)=N/O)c2oc(-c3cnn4cccnc34)c(C)c(=O)c2c1] | 504 [M + H]⁺ |
| 366C | 6-Chloro-3-[[(1R)-1-[3,6- dimethyl-4-oxo-2-[3- (trifluoromethyl)-1-bicyclo- [1.1.1]pentanyl]chromen-8- yl]ethyl]amino]-N′-hydroxy- pyridine-2-carboxamidine | 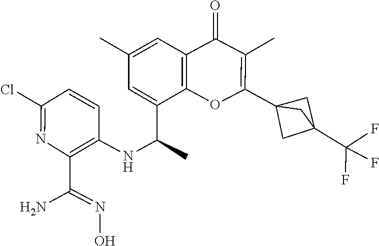 [SMILES: Cc1cc([C@@H](C)Nc2ccc(Cl)nc2/C(N)=N/O)c2oc(C34CC(C(F)(F)F)(C3)C4)c(C)c(=O)c2c1] | 521 [M + H]⁺ |
| 367C | 6-Chloro-3-[1-[6-fluoro-3- methyl-2-(1-methylpyrazol- 4-yl)-4-oxo-chromen-8-yl]- ethylamino]-N'-hydroxy- pyridine-2-carboxamidine, Isomer 1 | 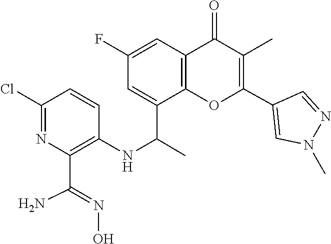 [SMILES: Cc1c(-c2cnn(C)c2)oc2c(C(C)Nc3ccc(Cl)nc3/C(N)=N/O)cc(F)cc2c1=O] | 471 [M + H]⁺ |
| 368C | 6-Chloro-3-[[(1R)-1-(2- cyclopropyl-3,6-dimethyl-4- oxo-chromen-8-yl)ethyl]- amino]-N′-hydroxy- pyridine-2-carboxamidine | 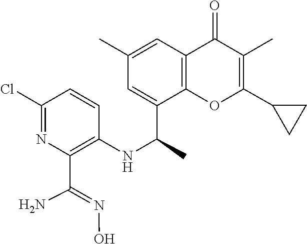 [SMILES: Cc1cc([C@@H](C)Nc2ccc(Cl)nc2/C(N)=N/O)c2oc(C3CC3)c(C)c(=O)c2c1] | 427 [M + H]⁺ |

¹NaHCO₃ used as the base in this reaction.

### Intermediate 36A: N-[[(4R)-2,2-Dimethyl-1,3-dioxolan-4-yl]methoxy]-2-[[(1R)-1-(3,6-dimethyl-4-oxo-2-phenyl-chromen-8-yl)ethyl]amino]benzamide

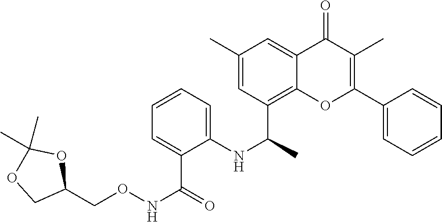 [SMILES: Cc1cc([C@@H](C)Nc2ccccc2C(=O)NOC[C@H]2COC(C)(C)O2)c2oc(-c3ccccc3)c(C)c(=O)c2c1]

To a solution of 2-[[(1R)-1-(3,6-dimethyl-4-oxo-2-phenyl-chromen-8-yl)ethyl]amino]benzoic acid (0.100 g, 0.242 mmol) in DMF (1 mL) was added (1-[bis(dimethylamino)methylene]-1H-1,2,3-triazolo[4,5-b]pyridinium 3-oxide hexafluorophosphate (0.276 g, 0.726 mmol), DIPEA (0.25 mL, 1.45 mmol), and J-O-((2,2-dimethyl-1,3-dioxolan-4-yl)methyl)hydroxylamine (0.107 g, 0.726 mmol). The reaction was stirred at rt for 24 h. Concentrated and purified by silica gel chromatography eluted with a gradient of 0 to 100% EtOAc in hexanes to give the title compound (0.171 g, 80% purity, 100%). MS ES+ m/z 543 [M+H]⁺.

The following compound in Table 40 was made in a similar way as described for N-[[(4R)-2,2-dimethyl-1,3-dioxolan-4-yl]methoxy]-2-[[(1R)-1-(3,6-dimethyl-4-oxo-2-phenyl-chromen-8-yl)ethyl]amino]benzamide.

TABLE 40

| | | | MS ES+ |
|---|---|---|---|
| Interm # | Chemical Name | Structure | m/z |
| 37A | 2-[[(1R)-1-(2- Ethylsulfanyl-3,6- dimethyl-4-oxo- chromen-8- yl)ethyl]amino]-N- methyl-benzamide | 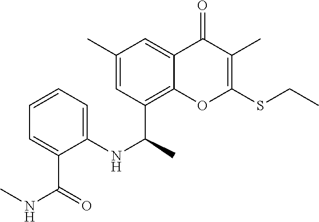 [SMILES: CCSc1oc2c([C@@H](C)Nc3ccccc3C(=O)NC)cc(C)cc2c(=O)c1C] | 411 [M + H]⁺ |

### Intermediate 38A: 2-[[(1R)-1-[2-(2-Fluorophenyl)-3,6-dimethyl-4-oxo-chromen-8-yl]ethyl]amino]benzonitrile

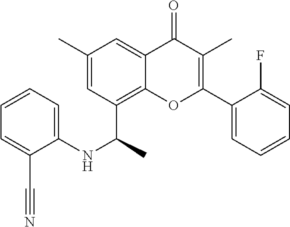 [SMILES: Cc1cc([C@@H](C)Nc2ccccc2C#N)c2oc(-c3ccccc3F)c(C)c(=O)c2c1]

Combined 2-[[(1R)-1-(2-ethylsulfanyl-3,6-dimethyl-4-oxo-chromen-8-yl)ethyl]amino]benzonitrile (0.100 g, 0.264 mmol), (2-fluorophenyl)boronic acid (0.111 g, 0.793 mmol), tetrakis(triphenylphosphine)palladium(0) (0.046 g, 0.040 mmol), and copper(I) thiophene-2-carboxylate (0.076 g, 0.396 mmol) in ethanol (15 mL). Degassed the reaction with argon and stirred at 50° C. for 16 h. Concentrated and purified by silica gel chromatography eluted with a gradient of 0 to 100% EtOAc in hexanes to give the title compound (0.109 g, 100%). MS ES+ m/z 413 [M+H]⁺.

The following compounds in Table 41 were made in a similar way as described for 2-[[(1R)-1-[2-(2-fluorophenyl)-3,6-dimethyl-4-oxo-chromen-8-yl]ethyl]amino]benzonitrile. Various methods were used to purify the compounds, which would be apparent to one skilled in the art.

TABLE 41

| | | | MS ES+ |
|---|---|---|---|
| Interm # | Chemical Name | Structure | m/z |
| 39A | 2-[[(1R)-1-[2-(3- Fluorophenyl)-3,6- dimethyl-4-oxo- chromen-8- yl]ethyl]amino]benzoni- trile | 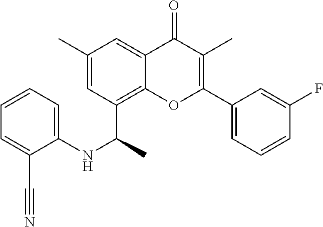 [SMILES: Cc1cc([C@@H](C)Nc2ccccc2C#N)c2oc(-c3cccc(F)c3)c(C)c(=O)c2c1] | 413 [M + H]⁺ |
| 40A | 2-[[(1R)-1-[3,6- Dimethyl-2-(2- methylindazol-5-yl)-4- oxo-chromen-8- yl]ethyl]amino]benzoni- trile | 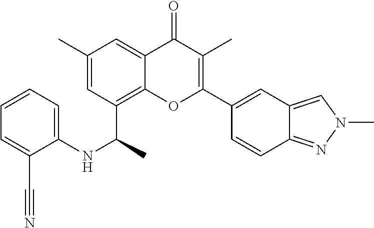 [SMILES: Cc1cc([C@@H](C)Nc2ccccc2C#N)c2oc(-c3ccc4nn(C)cc4c3)c(C)c(=O)c2c1] | 449 [M + H]⁺ |
| 41A | 2-[[(1R)-1-[3,6- Dimethyl-4-oxo-2-(3- pyridyl)chromen-8- yl]ethyl]amino]benzoni- trile | 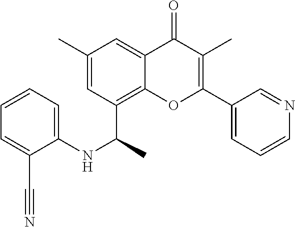 [SMILES: Cc1cc([C@@H](C)Nc2ccccc2C#N)c2oc(-c3cccnc3)c(C)c(=O)c2c1] | 396 [M + H]⁺ |
| 42A | 2-[[(1R)-1-[3,6- Dimethyl-4-oxo-2-(2- pyridyl)chromen-8- yl]ethyl]amino]benzoni- trile | 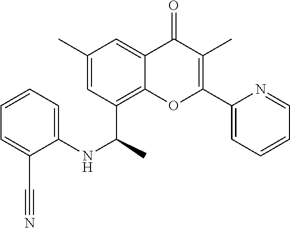 [SMILES: Cc1cc([C@@H](C)Nc2ccccc2C#N)c2oc(-c3ccccn3)c(C)c(=O)c2c1] | 396 [M + H]⁺ |
| 43A | tert-Butyl N-[(1R)-1- [3,6-dimethyl-4-oxo-2- (3-pyridyl)chromen-8- yl]ethyl]carbamate | 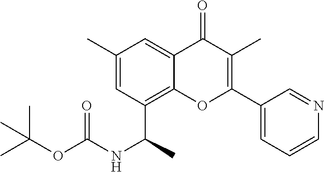 [SMILES: Cc1cc([C@@H](C)NC(=O)OC(C)(C)C)c2oc(-c3cccnc3)c(C)c(=O)c2c1] | 395 [M + H]⁺ |
| 44A | 8-[(1R)-1- Aminoethyl]-2-(3,4- difluorophenyl)-3,6- dimethyl-chromen-4- one | 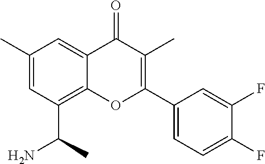 [SMILES: Cc1cc([C@@H](C)N)c2oc(-c3ccc(F)c(F)c3)c(C)c(=O)c2c1] | 330 [M + H]⁺ |
| 45A | 8-[(1R)-1- Aminoethyl]-3,6- dimethyl-2-(1- methylpyrazol-4- yl)chromen-4-one | 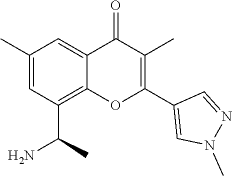 [SMILES: Cc1cc([C@@H](C)N)c2oc(-c3cnn(C)c3)c(C)c(=O)c2c1] | |
| 46A | 8-[(1R)-1- Aminoethyl]-3-methyl- 2-phenyl-6- (trifluoromethyl)chromen- 4-one | 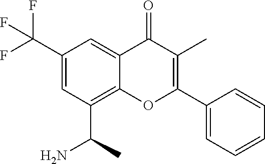 [SMILES: Cc1c(-c2ccccc2)oc2c([C@@H](C)N)cc(C(F)(F)F)cc2c1=O] | 348 [M + H]⁺ |
| 287A | 2-Fluoro-6-[[(1R)-1-[2- (2-fluorophenyl)-3,6- dimethyl-4-oxo- chromen-8- yl]ethyl]amino]benzoni- trile | 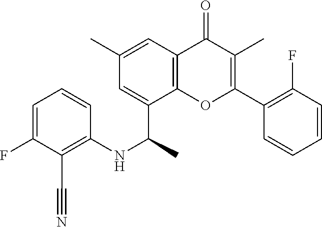 [SMILES: Cc1cc([C@@H](C)Nc2cccc(F)c2C#N)c2oc(-c3ccccc3F)c(C)c(=O)c2c1] | 431 [M + H]⁺ |
| 288A | tert-Butyl 6-chloro-3- [[(1R)-1-[2-(2- fluorophenyl)-3,6- dimethyl-4-oxo- chromen-8- yl]ethyl]amino]pyridine- 2-carboxylate | 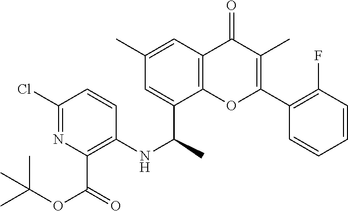 [SMILES: Cc1cc([C@@H](C)Nc2ccc(Cl)nc2C(=O)OC(C)(C)C)c2oc(-c3ccccc3F)c(C)c(=O)c2c1] | 467 [M + 2H − tBu]⁺ |
| 289A | 5-Fluoro-2-[[(1R)-1-[2- (2-fluorophenyl)-3,6- dimethyl-4-oxo- chromen-8- yl]ethyl]amino]benzoni- trile | 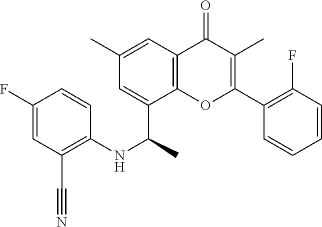 [SMILES: Cc1cc([C@@H](C)Nc2ccc(F)cc2C#N)c2oc(-c3ccccc3F)c(C)c(=O)c2c1] | 431 [M + H]⁺ |
| 290A | 8-[(1R)-1-[(2-Chloro- 3-pyridyl)amino]ethyl]- 3,6-dimethyl-2-(3- pyridyl)chromen-4-one | 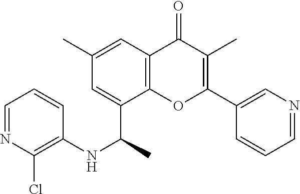 [SMILES: Cc1cc([C@@H](C)Nc2cccnc2Cl)c2oc(-c3cccnc3)c(C)c(=O)c2c1] | 406 [M + H]⁺ |
| 291A | tert-Butyl 6-chloro-3- [[(1R)-1-[2-(3- cyanophenyl)-3,6- dimethyl-4-oxo- chromen-8- yl]ethyl]amino]pyridine- 2-carboxylate | 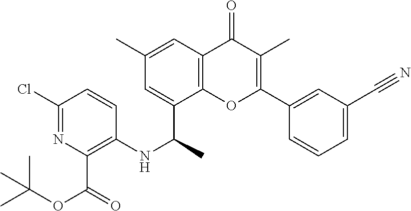 [SMILES: Cc1cc([C@@H](C)Nc2ccc(Cl)nc2C(=O)OC(C)(C)C)c2oc(-c3cccc(C#N)c3)c(C)c(=O)c2c1] | 528 [M + H]⁺ |
| 292A | 4-Fluoro-2-[[(1R)-1-[2- (2-fluorophenyl)-3,6- dimethyl-4-oxo- chromen-8- yl]ethyl]amino]benzoni- trile | 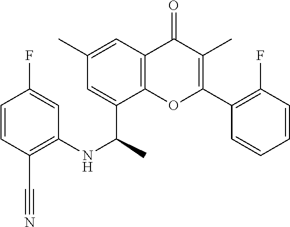 [SMILES: Cc1cc([C@@H](C)Nc2cc(F)ccc2C#N)c2oc(-c3ccccc3F)c(C)c(=O)c2c1] | 431 [M + H]⁺ |
| 293A | 6-Chloro-3-[[(1R)-1- [3,6-dimethyl-2-(1- methylimidazol-4-yl)- 4-oxo-chromen-8- yl]ethyl]amino]pyridine- 2-carboxamide | 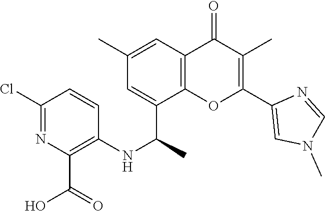 [SMILES: Cc1cc([C@@H](C)Nc2ccc(Cl)nc2C(=O)O)c2oc(-c3cn(C)cn3)c(C)c(=O)c2c1] | 453 [M + H]⁺ |

### Intermediate 47A: 8-[(1R)-1-Aminoethyl]-3,6-dimethyl-2-(3-pyridyl)chromen-4-one

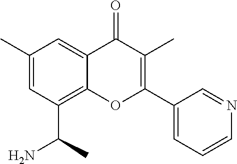 [SMILES: Cc1cc([C@@H](C)N)c2oc(-c3cccnc3)c(C)c(=O)c2c1]

To a solution of tert-butyl N-[(1R)-1-[3,6-dimethyl-4-oxo-2-(3-pyridyl)chromen-8-yl]ethyl]carbamate (0.950 g, 2.41 mmol) in DCM (24 mL) was added HCl (4M in 1,4-dioxane, 4 mL, 16 mmol). The reaction was stirred at rt for 2 h. Added HCl (4M in 1,4-dioxane, 1.2 mL, 4.8 mmol) and stirred at rt for 1 h. Concentrated and diluted with saturated aqueous NaHCO₃ and IPA:CHCl₃ (1:3). Separated the layers. The organic extracts were dried over MgSO₄, filtered, and concentrated. The residue was purified by reverse phase chromatography eluted with a gradient of 0% to 100% ACN in aqueous NH₄HCO₃ (10 mM, plus 5% MeOH). Clean fractions were washed with saturated aqueous sodium chloride and extracted with IPA:CHCl₃ (1:3). The organic extracts were dried over MgSO₄, filtered, and concentrated to give the title compound (0.364 g, 51%). MS ES+ m/z 295 [M+H]⁺.

The following compounds in Table 42 were made in a similar way as described for tert-butyl 3-[[(1R)-1-(3,6-dimethyl-4-oxo-2-phenyl-chromen-8-yl)ethyl]amino]pyridine-2-carboxylate. Various methods were used to purify the compounds, which would be apparent to one skilled in the art.

TABLE 42

| | | | MS ES+ |
|---|---|---|---|
| Interm # | Chemical Name | Structure | m/z |
| 48A | 3-[[(1R)-1-[3,6- Dimethyl-4-oxo-2-(3- pyridyl)chromen-8- yl]ethyl]amino]pyridine- 2-carbonitrile | 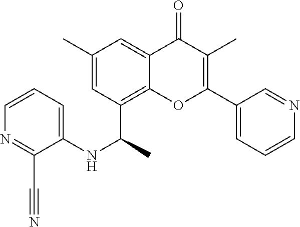 [SMILES: Cc1cc([C@@H](C)Nc2cccnc2C#N)c2oc(-c3cccnc3)c(C)c(=O)c2c1] | 397 [M + H]⁺ |
| 49A | 3-[[(1R)-1-[2-(3,4- Difluorophenyl)-3,6- dimethyl-4-oxo- chromen-8- yl]ethyl]amino]pyridine- 2-carbonitrile | 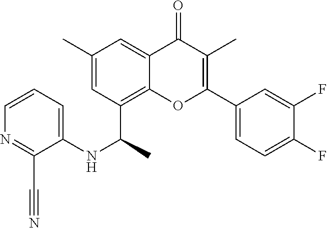 [SMILES: Cc1cc([C@@H](C)Nc2cccnc2C#N)c2oc(-c3ccc(F)c(F)c3)c(C)c(=O)c2c1] | 432 [M + H]⁺ |
| 50A | 8-[(1R)-1-(2- Bromoanilino)ethyl]- 3,6-dimethyl-2-(3- pyridyl)chromen-4-one | 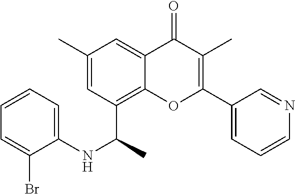 [SMILES: Cc1cc([C@@H](C)Nc2ccccc2Br)c2oc(-c3cccnc3)c(C)c(=O)c2c1] | 451 [M + H]⁺ |
| 51A | 2-[[(1R)-1-[3,6- Dimethyl-2-(1- methylpyrazol-4-yl)-4- oxo-chromen-8- yl]ethyl]amino]benzoni- trile | 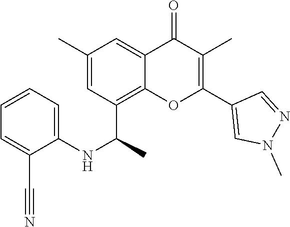 [SMILES: Cc1cc([C@@H](C)Nc2ccccc2C#N)c2oc(-c3cnn(C)c3)c(C)c(=O)c2c1] | |
| 52A | 3-[[(1R)-1-[3-Methyl- 4-oxo-2-phenyl-6- (trifluoromethyl)chromen- 8- yl]ethyl]amino]pyridine- 2-carbonitrile | 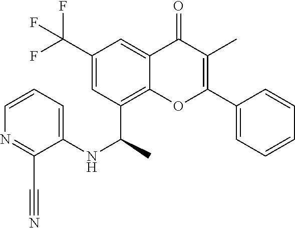 [SMILES: Cc1c(-c2ccccc2)oc2c([C@@H](C)Nc3cccnc3C#N)cc(C(F)(F)F)cc2c1=O] | 450 [M + H]⁺ |

### Intermediate 369C: 3-[3-[[(1R)-1-[2-[1-[2-[tert-Butyl(dimethyl)silyl]oxyethyl]pyrazol-4-yl]-3,6-dimethyl-4-oxo-chromen-8-yl]ethyl]amino]-6-chloro-2-pyridyl]-4H-1,2,4-oxadiazol-5-one

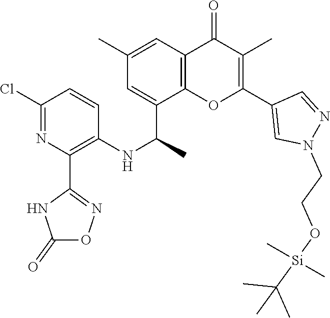 [SMILES: Cc1cc([C@@H](C)Nc2ccc(Cl)nc2-c2noc(=O)[nH]2)c2oc(-c3cnn(CCO[Si](C)(C)C(C)(C)C)c3)c(C)c(=O)c2c1]

A solution of 3-[[(1R)-1-[2-[1-[2-[tert-butyl(dimethyl)silyl]oxyethyl]pyrazol-4-yl]-3,6-dimethyl-4-oxo-chromen-8-yl]ethyl]amino]-6-chloro-N′-hydroxy-pyridine-2-carboxamidine (134 mg, 0.22 mmol) in 1,4-dioxane (4 mL) was treated with DBU (66.8 mg, 0.44 mmol) and 1,1′-carbonyldiimidazole (53.3 mg, 0.33 mmol). The reaction was stirred at 80° C. for 1 h. The reaction was cooled to rt and concentrated under reduced pressure. The residue was purified by silica gel chromatography eluted with 0% to 100% EtOAc/EtOH (3:1) in heptane to give the title compound (98.4 mg, 70%) as a pale tan foam. ES/MS m/z 637 (M+H).

The following compounds in Table 43 were made in a similar way as described for 3-[3-[[(1R)-1-[2-[1-[2-[tert-butyl(dimethyl)silyl]oxyethyl]pyrazol-4-yl]-3,6-dimethyl-4-oxo-chromen-8-yl]ethyl]amino]-6-chloro-2-pyridyl]-4H-1,2,4-oxadiazol-5-one. Various methods were used to purify the compounds, which would be apparent to one skilled in the art.

TABLE 43

| | | | MS ES+ |
|---|---|---|---|
| Interm # | Chemical Name | Structure | m/z |
| 370C | 3-[6-Chloro-3-[[(1R)-1-[3- iodo-6-methyl-4-oxo-2-(2- pyridyl)chromen-8- yl]ethyl]amino]-2-pyridyl]- 4H-1,2,4-oxadiazol-5-one | 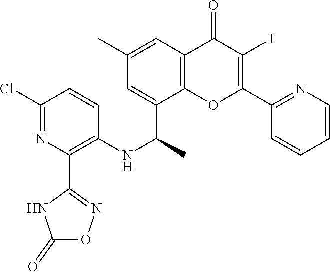 [SMILES: Cc1cc([C@@H](C)Nc2ccc(Cl)nc2-c2noc(=O)[nH]2)c2oc(-c3ccccn3)c(I)c(=O)c2c1] | 602 [M + H]⁺ |
| 371C | tert-Butyl 3-[4-[8-[(1R)-1- [[6-chloro-2-(5-oxo-4H- 1,2,4-oxadiazol-3-yl)-3- pyridyl]amino]ethyl]-3,6- dimethyl-4-oxo-chromen-2- yl]pyrazol-1-yl]azetidine-1- carboxylate | 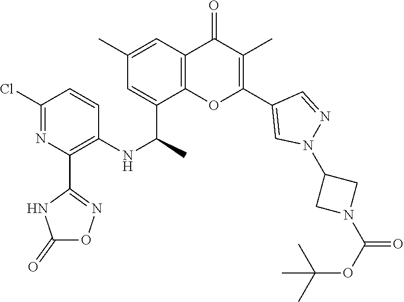 [SMILES: Cc1cc([C@@H](C)Nc2ccc(Cl)nc2-c2noc(=O)[nH]2)c2oc(-c3cnn(C4CN(C(=O)OC(C)(C)C)C4)c3)c(C)c(=O)c2c1] | 634 [M + H]⁺ |
| 372C | 3-[6-Chloro-3-[[(1R)-1-[2- [1-[(2,2-dimethyl-1,3- dioxolan-4- yl)methyl]pyrazol-4-yl]-3,6- dimethyl-4-oxo-chromen-8- yl]ethyl]amino]-2-pyridyl]- 4H-1,2,4-oxadiazol-5-one | 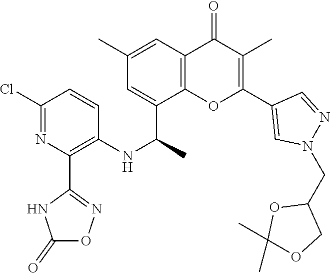 [SMILES: Cc1cc([C@@H](C)Nc2ccc(Cl)nc2-c2noc(=O)[nH]2)c2oc(-c3cnn(CC4COC(C)(C)O4)c3)c(C)c(=O)c2c1] | 593 [M + H]⁺ |

### Intermediate 294A: 6-Chloro-3-[[(1R)-1-[2-(2-fluorophenyl)-3,6-dimethyl-4-oxo-chromen-8-yl]ethyl]amino]pyridine-2-carboxylic acid

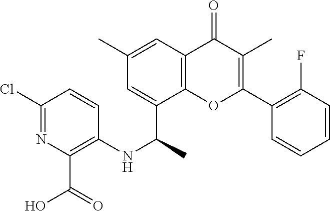 [SMILES: Cc1cc([C@@H](C)Nc2ccc(Cl)nc2C(=O)O)c2oc(-c3ccccc3F)c(C)c(=O)c2c1]

A solution of tert-butyl 6-chloro-3-[[(1R)-1-[2-(2-fluorophenyl)-3,6-dimethyl-4-oxo-chromen-8-yl]ethyl]amino]pyridine-2-carboxylate (0.23 g, 0.44 mmol) in 2 mL of DCM was treated with TFA/DCM (1:1; 1 mL) and the reaction stirred at 50° C. for 2 h. The reaction was concentrated and the residue purified by reversed phase C18 flash chromatography eluted with 0% to 100% ACN in 0.1% aqueous formic acid. Fractions containing the product were combined, washed with saturated aqueous sodium chloride, and extracted with IPA/CHCl₃ (1:3). The organics were combined, dried over MgSO₄, filtered, and concentrated to give the title compound (68.8 mg, 34%) as a tan foam. MS ES+ m/z 467 [M+H]⁺.

The following compounds in Table 44 was made in a similar way as described for 6-chloro-3-[[(1R)-1-[2-(2-fluorophenyl)-3,6-dimethyl-4-oxo-chromen-8-yl]ethyl]amino]pyridine-2-carboxylic acid.

TABLE 44

| | | | MS ES+ |
|---|---|---|---|
| Interm # | Chemical Name | Structure | m/z |
| 295A | 6-Chloro-3-[[(1R)-1- [2-(3-cyanophenyl)- 3,6-dimethyl-4-oxo- chromen-8- yl]ethyl]amino]pyridine- 2-carboxylic acid | 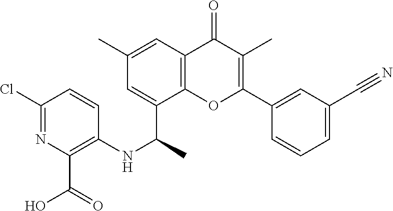 [SMILES: Cc1cc([C@@H](C)Nc2ccc(Cl)nc2C(=O)O)c2oc(-c3cccc(C#N)c3)c(C)c(=O)c2c1] | 474 [M + H]⁺ |
| 373C | 6-Chloro-3-[[(1R)-1- (3-iodo-6-methyl-4- oxo-2-phenyl-chromen- 8- yl)ethyl]amino]pyridine- 2-carboxylic acid | 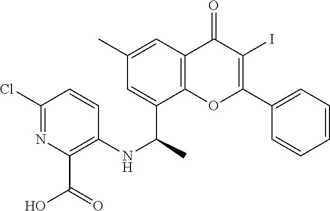 [SMILES: Cc1cc([C@@H](C)Nc2ccc(Cl)nc2C(=O)O)c2oc(-c3ccccc3)c(I)c(=O)c2c1] | 561 [M + H]⁺ |
| 374C | 3-[3-[[(1R)-1-[2-[1- (Azetidin-3-yl)pyrazol- 4-yl]-3,6-dimethyl-4- oxo-chromen-8- yl]ethyl]amino]-6- chloro-2-pyridyl]-4H- 1,2,4-oxadiazol-5-one | 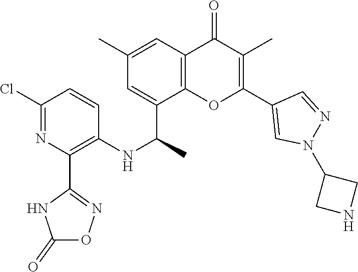 [SMILES: Cc1cc([C@@H](C)Nc2ccc(Cl)nc2-c2noc(=O)[nH]2)c2oc(-c3cnn(C4CNC4)c3)c(C)c(=O)c2c1] | 534 [M + H]⁺ |

### Intermediate 296A: 3-[[(1R)-1-[3,6-Dimethyl-2-(2-methylindazol-5-yl)-4-oxo-chromen-8-yl]ethyl]amino]-6-methyl-pyridine-2-carbohydrazide

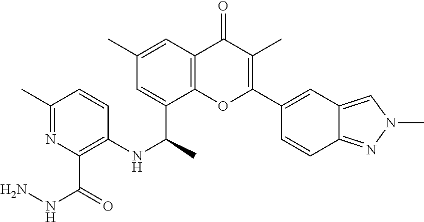

A solution of tert-butyl N-[[3-[[(1R)-1-[3,6-dimethyl-2-(2-methylindazol-5-yl)-4-oxo-chromen-8-yl]ethyl]amino]-6-methyl-pyridine-2-carbonyl]amino]carbamate (0.50 g, 0.84 mmol) in 10 mL of DCM was treated with HCl (4M in 1,4-dioxane, 0.15 g, 1.05 mL, 4.19 mmol) and stirred at 50° C. After 3 h, added another 1.05 mL of HCl in dioxane and stirred at 60° C. for 2 h. The reaction was allowed to cool to rt, concentrated, the residue taken up in DCM, and washed with saturated aqueous NaHCO₃. The aqueous layer was extracted twice with IPA/CHCl₃ (1:3). The organics were combined, dried over MgSO₄, filtered, and concentrated to a residue which was recrystallized from DCM/MTBE/hexanes to give the title compound (0.31 g, 73%) as a pale yellow powder. MS ES+ m/z 497 [M+H]⁺.

The following compounds in Table 45 were made in a similar way as described for 3-[[(1R)-1-[3,6-dimethyl-2-(2-methylindazol-5-yl)-4-oxo-chromen-8-yl]ethyl]amino]-6-methyl-pyridine-2-carbohydrazide. Various methods were used to purify the compounds, which would be apparent to one skilled in the art.

TABLE 45

| | | | MS ES+ |
|---|---|---|---|
| Interm # | Chemial Name | Structure | m/z |
| 297A | 3-[[(1R)-1-(3,6- Dimethyl-4-oxo-2- phenyl-chromen-8- yl)ethyl]amino]-6- methyl-pyridine-2- carbohydrazide | 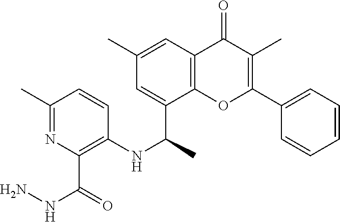 [SMILES: Cc1cc([C@@H](C)Nc2ccc(C)nc2C(=O)NN)c2oc(-c3ccccc3)c(C)c(=O)c2c1] | 443 [M + H]⁺ |
| 298A | 6-[[(1R)-1-(3,6- dimethyl-4-oxo-2- phenyl-chromen-8- yl)ethyl]amino]-2,3- difluoro- benzohydrazide | 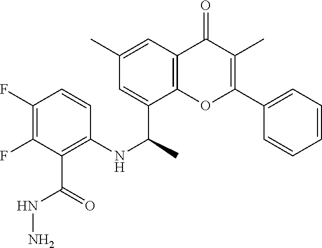 [SMILES: Cc1cc([C@@H](C)Nc2ccc(F)c(F)c2C(=O)NN)c2oc(-c3ccccc3)c(C)c(=O)c2c1] | 464 [M + H]⁺ |
| 299A | 3-[[(1R)-1-[2-(3,4- difluorophenyl)-3,6- dimethyl-4-oxo- chromen-8- yl]ethyl]amino]-6- methyl-pyridine-2- carbohydrazide | 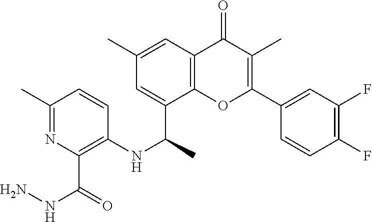 [SMILES: Cc1cc([C@@H](C)Nc2ccc(C)nc2C(=O)NN)c2oc(-c3ccc(F)c(F)c3)c(C)c(=O)c2c1] | 479 [M + H]⁺ |
| 300A | 2-[[(1R)-1-(3,6- dimethyl-4-oxo-2- phenyl-chromen-8- yl)ethyl]amino]-6- fluoro-benzohydrazide | 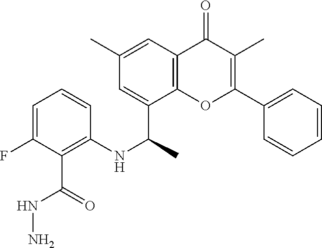 [SMILES: Cc1cc([C@@H](C)Nc2cccc(F)c2C(=O)NN)c2oc(-c3ccccc3)c(C)c(=O)c2c1] | 446 [M + H]⁺ |
| 301A | 3-[[(1R)-1-[2-[6- (Difluoromethyl)-2- pyridyl]-3,6-dimethyl- 4-oxo-chromen-8- yl]ethyl]amino]pyridine- 2-carbohydrazide | 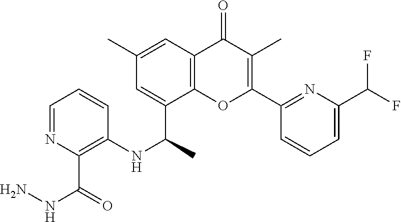 [SMILES: Cc1cc([C@@H](C)Nc2cccnc2C(=O)NN)c2oc(-c3cccc(C(F)F)n3)c(C)c(=O)c2c1] | |
| 302A | 2-[[(1R)-1-(6-Methyl- 4-oxo-2-phenyl-3- thiazol-5-yl-chromen- 8- yl)ethyl]amino]benzoic acid | 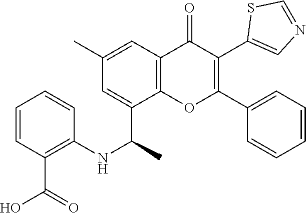 [SMILES: Cc1cc([C@@H](C)Nc2ccccc2C(=O)O)c2oc(-c3ccccc3)c(-c3cncs3)c(=O)c2c1] | 483 [M + H]⁺ |

### Intermediate 303A: 6-Chloro-3-[[(1R)-1-(3-iodo-6-methyl-4-oxo-2-phenyl-chromen-8-yl)ethyl]amino]-N-methylsulfonyl-pyridine-2-carboxamide

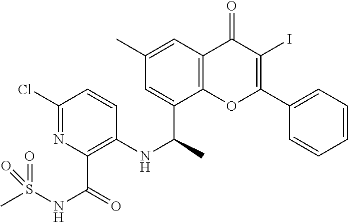 [SMILES: Cc1cc([C@@H](C)Nc2ccc(Cl)nc2C(=O)NS(C)(=O)=O)c2oc(-c3ccccc3)c(I)c(=O)c2c1]

6-Chloro-3-[[(1R)-1-(3-iodo-6-methyl-4-oxo-2-phenyl-chromen-8-yl)ethyl]amino]pyridine-2-carboxylic acid (0.14 g, 0.25 mmol), methanesulfonamide (47.5 mg, 0.50 mmol), DCC (0.10 g, 0.50 mmol) and DMAP (0.12 g, 1.0 mmol) were combined in DCM (2 mL) and allowed to stir at 25° C. overnight. The reaction was diluted with 1 mL of MeOH and directly purified by reversed phase chromatography eluted with 10% to 100% ACN (with 0.1% formic acid) in water (with 0.1% formic acid) to give the title compound (0.12 g, 75%). MS ES+ m/z 638 [M+H]⁺.

### Intermediate 375C: 6-Chloro-3-[[(1R)-1-(2-ethylsulfanyl-3,6-dimethyl-4-oxo-chromen-8-yl)ethyl]amino]-N-methylsulfonyl-pyridine-2-carboxamide

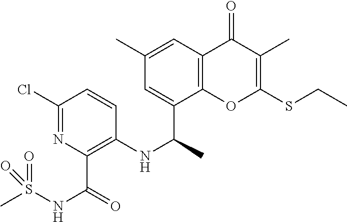 [SMILES: CCSc1oc2c([C@@H](C)Nc3ccc(Cl)nc3C(=O)NS(C)(=O)=O)cc(C)cc2c(=O)c1C]

A solution of 6-chloro-3-[[(1R)-1-(2-ethylsulfanyl-3,6-dimethyl-4-oxo-chromen-8-yl)ethyl]amino]pyridine-2-carboxylic acid (0.50 g, 1.15 mmol) in DCM (1.2 mL) was cooled to 0° C. and treated with methanesulfonamide (0.19 g, 1.96 mmol) and chlorodipyrrolidinecarbenium hexafluorophosphate (0.58 g, 1.73 mmol) followed by the dropwise addition of DIPEA (0.75 g, 5.77 mmol). The reaction was stirred at 0° C. for 5 min and then at rt over the weekend. Adsorbed the reaction onto silica gel and eluted with 0% to 10% MeOH in DCM to give the title compound (0.34 g, 58%). ES/MS m/z 510 (M+H).

The following compound in Table 46 was made in a similar way as described for 6-chloro-3-[[(1R)-1-(2-ethylsulfanyl-3,6-dimethyl-4-oxo-chromen-8-yl)ethyl]amino]-N-methylsulfonyl-pyridine-2-carboxamide. Various methods were used to purify the compound, which would be apparent to one skilled in the art.

TABLE 46

| | | | MS ES+ |
|---|---|---|---|
| Interm # | Chemical Name | Structure | m/z |
| 376C | 6-Chloro-3-[[(1R)-1- (3-iodo-6-methyl-4- oxo-2-phenyl-chromen- 8-yl)ethyl]amino]-N- methylsulfonyl- pyridine-2- carboxamide | 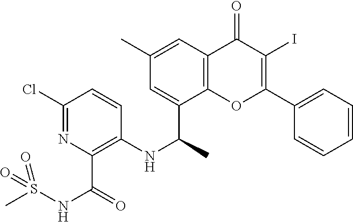 [SMILES: Cc1cc([C@@H](C)Nc2ccc(Cl)nc2C(=O)NS(C)(=O)=O)c2oc(-c3ccccc3)c(I)c(=O)c2c1] | 638 [M + H]⁺ |

### Intermediate 304A: 2-[[(1R)-1-(3,6-Dimethyl-4-oxo-2-phenyl-chromen-8-yl)ethyl]amino]benzenesulfonamide

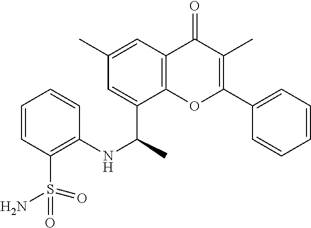 [SMILES: Cc1cc([C@@H](C)Nc2ccccc2S(N)(=O)=O)c2oc(-c3ccccc3)c(C)c(=O)c2c1]

A solution of N′-[2-[[(1R)-1-(3,6-dimethyl-4-oxo-2-phenyl-chromen-8-yl)ethyl]amino]phenyl]sulfonyl-N,N-dimethyl-formamidine (0.45 g, 0.89 mmol) in 8 mL of EtOH was treated with concentrated HCl (0.16 g, 4.45 mmol) and the reaction stirred at reflux for 2 h. Another 1 mL of concentrated HCl was added and the reaction stirred at 80° C. for another 2 h. The reaction was allowed to cool to rt, diluted with DCM, and neutralized with saturated aqueous NaHCO₃. The organic layer was removed and the aqueous layer extracted with IPA/CHCl₃ (1:3). The combined extracts were dried over MgSO₄, filtered, and concentrated. The residue was purified by reversed phase chromatography eluted with 0% to 100% ACN in water (with 0.1% formic acid). Fractions containing the product were pooled and extracted with IPA/CHCl₃ (1:3). The organics were combined, dried over MgSO₄, filtered, and concentrated to give the title compound (0.12 g, 30%) as a white solid. MS ES+ m/z 449 [M+H]⁺.

### Intermediate 53A: 3,6-Dimethyl-2-phenyl-8-[(1R)-1-[2-(4,4,5,5-tetramethyl-1,3,2-dioxaborolan-2-yl)anilino]ethyl]chromen-4-one

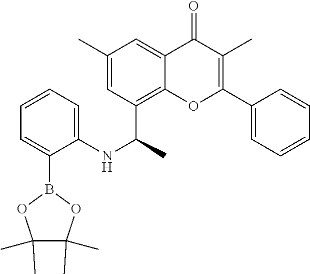 [SMILES: Cc1cc([C@@H](C)Nc2ccccc2B2OC(C)(C)C(C)(C)O2)c2oc(-c3ccccc3)c(C)c(=O)c2c1]

8-[(1R)-1-(2-Bromoanilino)ethyl]-3,6-dimethyl-2-phenyl-chromen-4-one (1.12 g, 2.50 mmol), potassium acetate (0.736 g, 7.49 mmol), 1,1′-bis(diphenylphosphino)ferrocenedichloro palladium(II) dichloromethane complex (0.183 g, 0.250 mmol), and bis(pinacolato)diboron (0.761 g, 3.00 mmol) were combined in dimethoxyethane (12 mL). The reaction was degassed with argon for 1 min and stirred at 95° C. for 16 h. Filtered through diatomaceous earth, concentrated, and purified by silica gel chromatography eluted with EtOAc in heptane to give the title compound (1.1 g, 89%). MS ES+ m/z 496 [M+H]⁺.

The following compound in Table 47 was made in a similar way as described for 3,6-dimethyl-2-phenyl-8-[(1R)-1-[2-(4,4,5,5-tetramethyl-1,3,2-dioxaborolan-2-yl)anilino]ethyl]chromen-4-one.

TABLE 47

| | | | MS ES+ |
|---|---|---|---|
| Interm # | Chemical Name | Structure | m/z |
| 54A | 3,6-Dimethyl-2-(3- pyridyl)-8-[(1R)-1-[2- (4,4,5,5-tetramethyl- 1,3,2-dioxaborolan-2- yl)anilino]ethyl]chromen- 4-one | 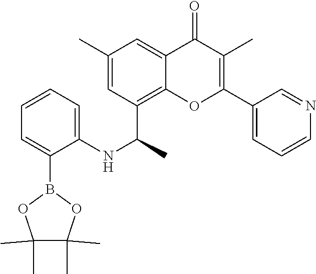 [SMILES: Cc1cc([C@@H](C)Nc2ccccc2B2OC(C)(C)C(C)(C)O2)c2oc(-c3cccnc3)c(C)c(=O)c2c1] | 497 [M + H]⁺ |

### Intermediate 55A: 2-Ethylsulfanyl-3,6-dimethyl-8-[(1R)-1-[2-(2H-tetrazol-5-yl)anilino]ethyl]chromen-4-one

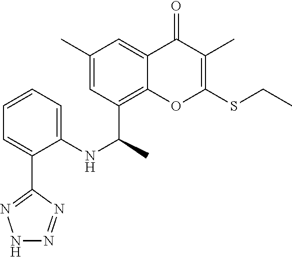 [SMILES: CCSc1oc2c([C@@H](C)Nc3ccccc3-c3nn[nH]n3)cc(C)cc2c(=O)c1C]

Combined 2-[[(1R)-1-(2-ethylsulfanyl-3,6-dimethyl-4-oxo-chromen-8-yl)ethyl]amino]benzonitrile (0.500 g, 1.32 mmol), sodium azide (0.258 g, 3.96 mmol), and ammonium chloride (0.212 g, 3.96 mmol) in DMF (3 mL). The reaction was sealed and stirred at 140° C. for 3 h. Purified by reverse phase chromatography to give the title compound (0.190 g, 34%). MS ES+ m/z 422 [M+H]⁺.

### Intermediate 56A: 3,6-Dimethyl-2-phenyl-8-[(1R)-1-[2-(2-trimethylsilylethynyl)anilino]ethyl]chromen-4-one

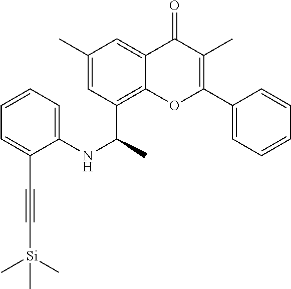 [SMILES: Cc1cc([C@@H](C)Nc2ccccc2C#C[Si](C)(C)C)c2oc(-c3ccccc3)c(C)c(=O)c2c1]

To a solution of 8-[(1R)-1-(2-bromoanilino)ethyl]-3,6-dimethyl-2-phenyl-chromen-4-one (0.35 g, 0.78 mmol) in DMF (4 mL) was added ethynyl-trimethyl-silane (0.46 g, 4.68 mmol) and triethylamine (0.65 mL, 4.68 mmol). The reaction was degassed with nitrogen and cuprous iodide (0.030 g, 0.16 mmol) and tetrakis(triphenylphosphine)palladium(0) (0.18 g, 0.16 mmol) were added. The vial was sealed under nitrogen and heated in a microwave reactor at 100° C. for 18 h. The mixture was diluted with 10% aqueous LiCl and extracted twice with EtOAc. The organic extracts were dried over Na₂SO₄, filtered, and concentrated. The residue was purified by silica gel chromatography eluted with a gradient of 0 to 100% EtOAc in hexanes to give the title compound (0.35 g, 70% purity, 67%). The crude material was used without further purification.

### Intermediate 57A: 8-[(1R)-1-(2-Ethynylanilino)ethyl]-3,6-dimethyl-2-phenyl-chromen-4-one

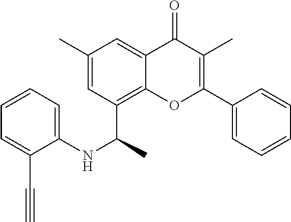 [SMILES: C#Cc1ccccc1N[C@H](C)c1cc(C)cc2c(=O)c(C)c(-c3ccccc3)oc12]

To a solution of 3,6-dimethyl-2-phenyl-8-[(1R)-1-[2-(2-trimethylsilylethynyl)anilino]ethyl]chromen-4-one (0.35 g, 70% purity, 0.52 mmol) in MeOH (4 mL) was added potassium carbonate (0.31 g, 2.25 mmol). The reaction was stirred at rt for 1 h. Filtered, washed with DCM, and concentrated. The residue was purified by silica gel chromatography eluted with a gradient of 0 to 60% EtOAc in hexanes to give the title compound (0.21 g, 90% purity, 90%). MS ES+ m/z 394 [M+H]⁺.

### Intermediate 305A: 8-[(1R)-1-(2-Dimethoxyphosphorylanilino)ethyl]-2-ethylsulfanyl-3,6-dimethyl-chromen-4-one

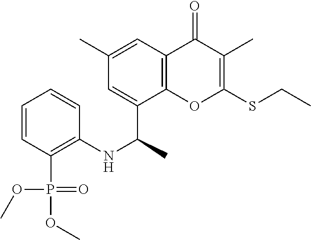 [SMILES: CCSc1oc2c([C@@H](C)Nc3ccccc3P(=O)(OC)OC)cc(C)cc2c(=O)c1C]

8-[(1R)-1-(2-Bromoanilino)ethyl]-2-ethylsulfanyl-3,6-dimethyl-chromen-4-one (0.10 g, 0.23 mmol), cesium carbonate (82.2 mg, 0.25 mmol), and tetrakis(triphenylphosphine)palladium(0) (12.1 mg, 0.01 mmol) were transferred to a microwave vial. The vial was evacuated and refilled with nitrogen three times before the addition of THF (0.5 mL) and phosphonic acid dimethyl ester (23.1 mg, 0.21 mmol). The vial was capped and the reaction stirred at 120° C. 10 min. The vial was recharged with phosphonic acid dimethyl ester (23.1 mg, 0.21 mmol) and the reaction heated at 120° C. for another 10 min. The reaction was adsorbed onto celite and purified by reversed phase chromatography eluted with 0% to 80% ACN in 10 mM NH₄HCO₃ (with 5% MeOH). MS ES+ m/z 462 [M+H]⁺.

### Intermediate 306A: 8-[[(1R)-1-[3,6-Dimethyl-4-oxo-2-(3-pyridyl)chromen-8-yl]ethyl]amino]-2-[(4-methoxyphenyl)methyl]-3,4-dihydroisoquinolin-1-one

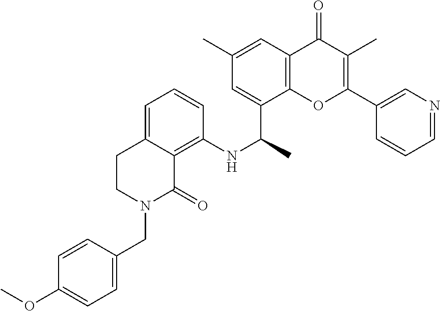 [SMILES: COc1ccc(CN2CCc3cccc(N[C@H](C)c4cc(C)cc5c(=O)c(C)c(-c6cccnc6)oc45)c3C2=O)cc1]

A 20 mL pressure vial was charged with 8-bromo-2-[(4-methoxyphenyl)methyl]-3,4-dihydroisoquinolin-1-one (177 mg, 0.51 mmol), 8-[(1R)-1-aminoethyl]-3,6-dimethyl-2-(3-pyridyl)chromen-4-one (150 mg, 0.51 mmol), cesium carbonate (333 mg, 1.02 mmol), BINAP (95.5 mg, 0.15 mmol), palladium(II) acetate (11.5 mg, 0.05 mmol), and 1,4-dioxane (5 mL). The mixture was degassed with nitrogen gas for 5 min and then stirred at 90° C. for 17 h. The reaction was passed through a syringe filter and purified by reversed phase chromatography eluted with 10% to 100% ACN in 10 mM aqueous NH₄HCO₃ to give the title compound (149 mg, 52%) a yellow semi-solid. MS ES+ m/z 560 [M+H]⁺.

The following compound in Table 48 was made in a similar way as described for 8-[[(1R)-1-[3,6-dimethyl-4-oxo-2-(3-pyridyl)chromen-8-yl]ethyl]amino]-2-[(4-methoxyphenyl)methyl]-3,4-dihydroisoquinolin-1-one.

TABLE 48

| | | | MS ES+ |
|---|---|---|---|
| Interm # | Chemical Name | Structure | m/z |
| 307A | 8-[[(1R)-1-[3,6- Dimethyl-4-oxo-2-(3- pyridyl)chromen-8- yl]ethyl]amino]-2-(2- trimethylsilylethoxymeth- yl)isoquinolin-1-one | 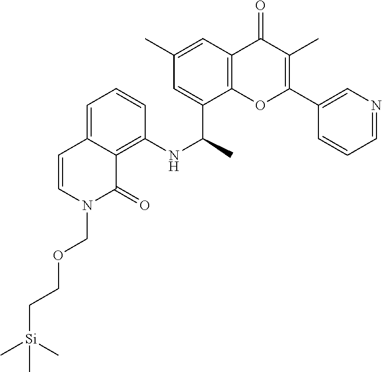 [SMILES: Cc1cc([C@@H](C)Nc2cccc3ccn(COCC[Si](C)(C)C)c(=O)c23)c2oc(-c3cccnc3)c(C)c(=O)c2c1] | 568 [M + H]⁺ |

### Intermediate 377C: tert-Butyl 2-[8-[(1R)-1-[[6-chloro-2-(methylsulfonylcarbamoyl)-3-pyridyl]amino]ethyl]-3,6-dimethyl-4-oxo-chromen-2-yl]indole-1-carboxylate

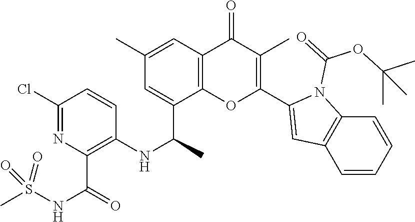 [SMILES: Cc1cc([C@@H](C)Nc2ccc(Cl)nc2C(=O)NS(C)(=O)=O)c2oc(-c3cc4ccccc4n3C(=O)OC(C)(C)C)c(C)c(=O)c2c1]

A vial was charged with 6-chloro-3-[[(1R)-1-(2-ethylsulfanyl-3,6-dimethyl-4-oxo-chromen-8-yl)ethyl]amino]-N-methylsulfonyl-pyridine-2-carboxamide (170 mg, 0.33 mmol), (1-tert-butoxycarbonylindol-2-yl)boronic acid (131 mg, 0.50 mmol), copper(I) 3-methylsalicylate (107 mg, 0.50 mmol), and tetrakis(triphenylphosphine)palladium(0) (77 mg, 0.07 mmol), and EtOH (5 mL). The reaction was degassed with argon, sealed, and stirred at 65° C. for 16 h. After cooling, the reaction was filtered and the filtrate concentrated under reduced pressure. The residue was purified by reversed phase chromatography on C18 eluted with 10% to 100% ACN (with 0.1% formic acid) in water (with 0.1% formic acid) to give the title compound (15 mg, 7%). ES/MS m/z 665 (M+H).

### Intermediate 1B: (2-Bromo-4-methyl-phenyl) propanoate

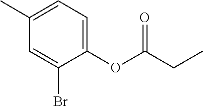

A mixture of 2-bromo-4-methyl-phenol (10.0 g, 53.5 mmol) and pyridine (6.34 g, 80.2 mmol) in DCM (100 mL) was treated with propanoyl chloride (5.44 g, 58.8 mmol) at 0° C. and stirred at 25° C. for 16 h. The mixture was diluted with water (100 mL), the pH adjusted to 5 with HCl (2M), and extracted with DCM (2×100 mL). The combined organic extracts were washed with saturated aqueous NaCl (2×150 mL), dried over anhydrous Na₂SO₄, filtered, and concentrated to give the product as an oil (13 g, crude). ¹H NMR (DMSO-d6) δ ppm 1.17 (t, J=7.6 Hz, 3H), 2.30 (s, 3H), 2.62 (q, J=7.6 Hz, 2H), 7.11-7.18 (m, 1H), 7.19-7.26 (m, 1H), 7.50-7.55 (m, 1H).

### Intermediate 2B: 1-(3-Bromo-2-hydroxy-5-methyl-phenyl)propan-1-one

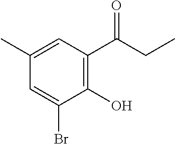

A mixture of (2-bromo-4-methyl-phenyl) propanoate (12.5 g, 51.4 mmol) and AlCl₃ (24.0 g, 180 mmol) was stirred at 140° C. for 1 h. When cooled to rt, the mixture was quenched with water (80 mL) dropwise and stirred for 30 min. The mixture was extracted with EtOAc (3×100 mL). The combined organic extracts were washed with saturated aqueous NaCl (2×200 mL), dried over anhydrous Na₂SO₄, and filtered. The filtrate was concentrated and triturated with petroleum ether (20 mL) to give the product as a solid (9.82 g, 79%). ¹H NMR (DMSO-d6) δ ppm 1.10 (t, J=7.2 Hz, 3H), 2.28 (s, 3H), 3.15 (q, J=7.2 Hz, 2H), 7.66-7.73 (m, 1H), 7.77-7.83 (m, 1H), 12.66 (s, 1H).

### Intermediate 18B: 8-Bromo-4-hydroxy-3,6-dimethyl-chromene-2-thione

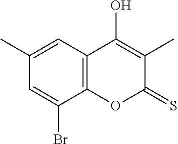

A mixture of 1-(3-bromo-2-hydroxy-5-methyl-phenyl)propan-1-one (1700 g, 6.99 mol), carbon disulfide (798.3 g, 10.49 mol), and THF (17 L) was cooled to −25° C. and treated with sodium bis(trimethylsilyl)amide (12.24 L, 2M) over 2 h. After addition was complete, warmed the reaction to 20-30° C. and stirred for 12 h. The reaction was cooled to 0° C. and quenched with sulfuric acid (17 L, 15%) over 2 h. The organic phase was removed and the aqueous layer extracted with EtOAc (1.7 L). The organic phases were combined, concentrated, and the resulting residue suspended in heptane giving the title compound (2000 g, 100%) as a yellow solid after filtration and drying. ¹H NMR (DMSO-d6) δ ppm 2.26 (s, 3H), 2.40 (s, 3H), 7.82 (m, 2H).

### Intermediate 3B: (E)-1-(3-Bromo-2-hydroxy-5-methyl-phenyl)-2-methyl-3-phenyl-prop-2-en-1-one

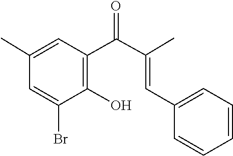 [SMILES: C/C(=C\c1ccccc1)C(=O)c1cc(C)cc(Br)c1O]

A mixture of 1-(3-bromo-2-hydroxy-5-methyl-phenyl)propan-1-one (200 g, 822.72 mmol), benzaldehyde (96.04 g, 904.99 mmol), AcOH (105.23 g, 1.75 mol), and piperidine (172.33 g, 2.02 mol) in EtOH (1600 mL) was stirred at 70° C. for 16 h. The resulting dark solution was poured into water (3 L), filtered, and the solid dissolved in 6 L of DCM. The organic solution was dried over anhydrous Na₂SO₄, filtered, and concentrated to give the product as a dark gum (284 g). MS ES+ m/z 331, 333 [M+H]⁺.

### Intermediate 4B: 8-Bromo-3,6-dimethyl-2-phenyl-chromen-4-one

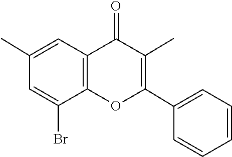 [SMILES: Cc1cc(Br)c2oc(-c3ccccc3)c(C)c(=O)c2c1]

A mixture of (E)-1-(3-bromo-2-hydroxy-5-methyl-phenyl)-2-methyl-3-phenyl-prop-2-en-1-one (284 g, 857.48 mmol) and 12 (21.76 g, 85.75 mmol) in DMSO (1200 mL) was stirred at 140° C. for 2 h to give a black-brown solution. Cooled to rt, poured the reaction into 3 L of water, filtered, dissolved the solid product in DCM (4 L), dried over anhydrous Na₂SO₄, filtered, and concentrated to give a residue. The residue was triturated with petroleum ether/EtOAc (1:1, 1 L) to give the product as a light yellow solid (195 g, 69%). MS ES+ m/z 329, 331 [M+H]⁺.

### Intermediate 19B: 8-Bromo-2-ethylsulfanyl-3,6-dimethyl-chromen-4-one

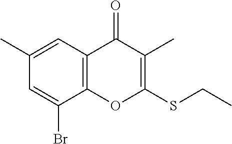 [SMILES: CCSc1oc2c(Br)cc(C)cc2c(=O)c1C]

A mixture of 8-bromo-4-hydroxy-3,6-dimethyl-chromene-2-thione (1900 g, 6.66 mol), 1-iodoethane (1558 g, 9.99 mol), potassium carbonate (920.9 g, 6.66 mol) in acetone (19 L) was stirred at 60° C. for 1 h. The reaction was cooled to rt, filtered, and the solids washed with THF. The filtrate was concentrated and the residue suspended in heptane (3.8 L) and the title compound (1900 g, 91%) was removed by filtration as a brown solid.

### Intermediate 5B: 8-Acetyl-3,6-dimethyl-2-phenyl-chromen-4-one

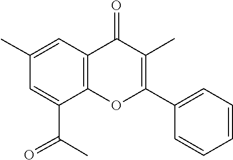 [SMILES: CC(=O)c1cc(C)cc2c(=O)c(C)c(-c3ccccc3)oc12]

A mixture of 8-bromo-3,6-dimethyl-2-phenyl-chromen-4-one (195 g, 592.37 mmol), bis(triphenylphosphine)palladium(II) dichloride (20.79 g, 29.62 mmol), and tributyl(1-ethoxyvinyl)stannane (256.72 g, 710.84 mmol, 239.92 mL) in dioxane (1600 mL) was stirred under N2 at 95° C. for 16 h to give a black-brown solution. After cooling to rt, treated the reaction with 1M aqueous HCl (100 mL) and stirred at 20° C. for 30 min. The mixture was quenched with saturated aqueous KF (2000 mL), stirred for 30 min, and filtered. The filter cake was washed with 10% MeOH in DCM (5×5000 mL). The combined extracts were dried over anhydrous Na₂SO₄, filtered, and concentrated to give a residue. The residue was triturated with petroleum ether/EtOAc (5/1, 1000 mL) to give a crude product which was triturated with DCM/MeOH (10/1, 500 mL) to give the product as a light yellow solid (180 g, 96%, 92% purity). MS ES+ m/z 293 [M+H]⁺.

The following compound in Table 49 were made in a similar way as described for 8-acetyl-3,6-dimethyl-2-phenyl-chromen-4-one. Various methods were used to purify the compound, which would be apparent to one skilled in the art.

TABLE 49

| | | | MS ES+ |
|---|---|---|---|
| Interm # | Chemical Name | Structure | m/z |
| 20B | 8-Acetyl-2- ethylsulfanyl-3,6- dimethyl-chromen-4- one | 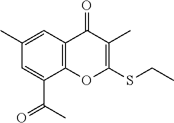 [SMILES: CCSc1oc2c(C(C)=O)cc(C)cc2c(=O)c1C] | |

### Intermediate 21B: 8-[(1R)-1-Hydroxyethyl]-3,6-dimethyl-2-phenyl-chromen-4-one

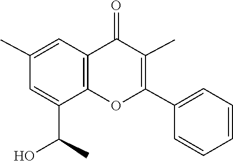 [SMILES: Cc1cc([C@@H](C)O)c2oc(-c3ccccc3)c(C)c(=O)c2c1]

A solution of 8-acetyl-3,6-dimethyl-2-phenyl-chromen-4-one (10 g, 34.2 mmol) and RuCl(p-cymene)[(R,R)-Ts-DPEN] (CAS 192139-92-7, 0.653 g, 1.03 mmol) in MeOH (50 mL) was stirred at 0-5° C. 1,8-Diazabicyclo[5.4.0]undec-7-ene (15.3 mL, 103 mmol) was added slowly, maintaining the temperature below 25° C. Formic acid (3.87 mL, 103 mmol) was added in portions, maintaining the temperature below 15° C. The reaction was stirred at 55° C. for 3 h, then cooled to rt. 4M aqueous HCl (50 mL) was slowly added. The resulting slurry was stirred overnight, filtered, and washed with water to give the title compound (9.9 g, 93%, 87% ee).

### Intermediate 378C: 8-[(1R)-1-Hydroxyethyl]-3,6-dimethyl-2-(3-pyridyl)chromen-4-one

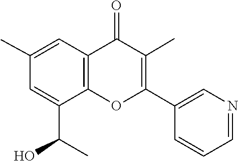 [SMILES: Cc1cc([C@@H](C)O)c2oc(-c3cccnc3)c(C)c(=O)c2c1]

A solution of 8-acetyl-3,6-dimethyl-2-(3-pyridyl)chromen-4-one (110 g, 375 mmol) in DCM (1.1 L) was cooled to 0 to 5° C. Formic acid (52 g, 1.13 mmol) was added in one portion followed by slow addition of triethylamine (76.5 g, 756 mmol) at 5 to 20° C. The reaction was cooled to 0 to 5° C. and treated with Noyori catalyst (CAS: 174813-82-2) (2.42 g, 3.81 mmol) in one portion. The reaction was stirred at rt for 22 h. The mixture was washed with saturated aqueous NaHCO₃ (2×5V) and water (2×5V). The organic phase was concentrated under reduced pressure and solvent swapped with MTBE. The resulting slurry was filtered and the solid rinsed with MTBE to give the title compound (122 g, 99%) as a yellow solid. ES/MS m/z 296 (M+H).

### Intermediate 22B: 8-[(1S)-1-Chloroethyl]-3,6-dimethyl-2-phenyl-chromen-4-one

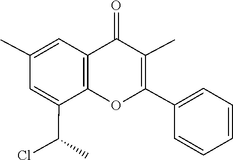 [SMILES: Cc1cc([C@H](C)Cl)c2oc(-c3ccccc3)c(C)c(=O)c2c1]

A solution of 8-[(1R)-1-hydroxyethyl]-3,6-dimethyl-2-phenyl-chromen-4-one (20 g, 68.0 mmol) in cyclopentyl methyl ether (200 mL) was stirred at room temperature. 2,4,6-trichloro[1,3,5]triazine (12.5 g, 68.0 mmol) was added, followed by DMF (7.9 mL, 102 mmol). The reaction was stirred at room temperature for 14 h. Diluted with 2M aqueous sodium hydroxide (100 mL) and separated the layers. The organics were washed with water (100 mL)/saturated aqueous sodium bicarbonate (100 mL) and 5% aqueous lithium chloride (100 mL). The organics were concentrated and diluted with IPA (120 mL). The slurry was heated at 45° C. for 2 h and cooled to room temperature. Water (80 mL) was slowly added. Filtered to give the title compound (18.4 g, 87%, 94% ee). MS ES+ m/z 313 [M+H]⁺.

### Intermediate 379C: 8-[(1S)-1-Chloroethyl]-3,6-dimethyl-2-(3-pyridyl)chromen-4-one

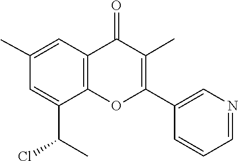 [SMILES: Cc1cc([C@H](C)Cl)c2oc(-c3cccnc3)c(C)c(=O)c2c1]

A solution of DMF (80 g, 1.09 mol) in DCM (550 mL) was treated with 2,4,6-trichloro-1,3,5-triazine (72 g, 390.43 mmol) at rt. The mixture was stirred for 2 h to form a white solid precipitate. A solution of 8-[(1R)-1-hydroxyethyl]-3,6-dimethyl-2-(3-pyridyl)chromen-4-one (110 g, 372.5 mmol) in DCM (550 mL) was added and the solution stirred at rt for 14 h. The reaction was quenched by adding water (1100 mL) and stirred for 30 min. The mixture was filtered through diatomaceous earth and cake rinsed with DCM (220 mL). The filtrate was washed with saturated aqueous NaHCO₃ (1100 mL) and water (1100 mL). The organic layer was collected, dried over MgSO₄, and concentrated under reduced pressure. The residue was purified by silica gel chromatography eluted with 25% THF in heptane to give a yellow solid (29 g, 93.9% ee). ES/MS m/z 314 (M+H).

### Intermediate 6B: (NE,R)—N-[1-(3,6-Dimethyl-4-oxo-2-phenyl-chromen-8-yl)ethylidene]-2-methyl-propane-2-sulfinamide

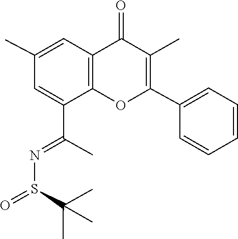 [SMILES: C/C(=N\[S@](=O)C(C)(C)C)c1cc(C)cc2c(=O)c(C)c(-c3ccccc3)oc12]

To a mixture of 8-acetyl-3,6-dimethyl-2-phenyl-chromen-4-one (180 g, 615.75 mmol) and (R)-2-methylpropane-2-sulfinamide (149.26 g, 1.23 mol) in THF (1500 mL) was added tetraisopropoxytitanium (700.01 g, 2.46 mol, 726.90 mL). The mixture was stirred at 80° C. for 56 h to give a black-brown solution. After cooling to rt, quenched the reaction with saturated aqueous NaCl (2000 mL) and stirred for 30 min and filtered. The filter cake was washed with EtOAc (4000 mL). After separating the organic layer, the aqueous layer was extracted with EtOAc (1000 mL). The combined organic extracts were dried over anhydrous Na₂SO₄, filtered, and concentrated to give a residue. The residue was triturated with petroleum ether/EtOAc (1/1, 600 mL) to give the product as a white solid (186 g, 76%). MS ES+ m/z 396 [M+H]⁺.

### Intermediate 7B: (R)—N-[(1R)-1-(3,6-Dimethyl-4-oxo-2-phenyl-chromen-8-yl)ethyl]-2-methyl-propane-2-sulfinamide

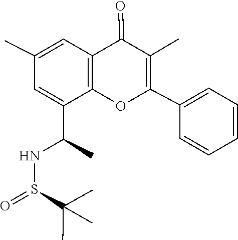 [SMILES: Cc1cc([C@@H](C)N[S@](=O)C(C)(C)C)c2oc(-c3ccccc3)c(C)c(=O)c2c1]

To a mixture of (NE,R)—N-[1-(3,6-dimethyl-4-oxo-2-phenyl-chromen-8-yl)ethylidene]-2-methyl-propane-2-sulfinamide (186 g, 470.27 mmol) and CeCl₃·7H₂O (87.61 g, 235.14 mmol, 22.35 mL) in MeOH (1600 mL) was added NaBH₄ (26.69 g, 705.41 mmol) at 15° C. The mixture was stirred at 15° C. for 1 h to give a dark suspension. The reaction was quenched with saturated aqueous NH₄Cl (1500 mL) at 15° C. Extracted with DCM (2×1500 mL), washed the combined organic phases with saturated aqueous NaCl (1500 mL), dried the organic phase over anhydrous Na₂SO₄, filtered, and concentrated to give the product as a yellow solid (180 g, 96%). MS ES+ m/z 398 [M+H]⁺.

### Intermediate 8B: 8-[(1R)-1-Aminoethyl]-3,6-dimethyl-2-phenyl-chromen-4-one

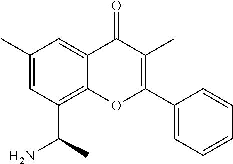 [SMILES: Cc1cc([C@@H](C)N)c2oc(-c3ccccc3)c(C)c(=O)c2c1]

A mixture of (R)—N-[(1R)-1-(3,6-dimethyl-4-oxo-2-phenyl-chromen-8-yl)ethyl]-2-methyl-propane-2-sulfinamide (180 g, 452.80 mmol) in MeOH (1500 mL) was treated with 4M HCl/MeOH (4M, 300 mL) and the mixture was stirred at 15° C. for 1 h to give a white suspension. Concentrated the reaction, poured the residue into water (1000 mL) and DCM (2000 mL), adjusted the pH to 12 with NH₃ in H₂O (25%), and extracted with DCM (2×1000 mL). The combined organic phases were washed with saturated aqueous NaCl (1000 mL), dried over anhydrous Na₂SO₄, filtered, and concentrated to give a residue. The residue was triturated with DCM (200 mL) to give the product as a white solid (122 g, 89%). MS ES+ m/z 294 [M+H]⁺.

### Intermediate 9B: (R)—N-[(1R)-1-(2-Ethylsulfanyl-3,6-dimethyl-4-oxo-chromen-8-yl)ethyl]-2-methyl-propane-2-sulfinamide

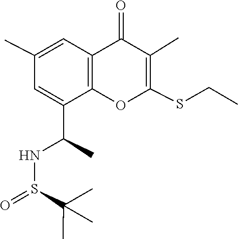 [SMILES: CCSc1oc2c([C@@H](C)N[S@](=O)C(C)(C)C)cc(C)cc2c(=O)c1C]

A mixture of 8-acetyl-2-ethylsulfanyl-3,6-dimethyl-chromen-4-one (50 g, 0.18 mol) in 500 mL of toluene was treated with (R)-2-methylpropane-2-sulfinamide (32.7 g, 0.27 mol) and titanium ethoxide (82.1 g, 0.36 mol) in one portion. The reaction was heated at 80° C. for 24 h, then cooled to rt.

The toluene solution of (NE,R)—N-[1-(2-ethylsulfanyl-3,6-dimethyl-4-oxo-chromen-8-yl)ethylidene]-2-methyl-propane-2-sulfinamide (0.18 mol) was cooled to between −10° C. and 0° C. and treated in portions with sodium borohydride (20.4 g, 0.54 mmol) keeping the internal temperature below 0° C. After stirring at −10° C. and 0° C. for 1 h, the reaction was quenched with saturated aqueous NH₄Cl. The resulting white slurry was filtered through diatomaceous earth and the solids washed with THF. The filtrate was washed with saturated aqueous NaCl and the organic layer concentrated onto silica gel. The material was purified by chromatography to give the product (26 g, 37% over two steps). MS ES+ m/z 382 [M+H]⁺.

### Intermediate 10B: 8-[(1R)-1-Aminoethyl]-2-ethylsulfanyl-3,6-dimethyl-chromen-4-one

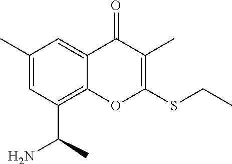 [SMILES: CCSc1oc2c([C@@H](C)N)cc(C)cc2c(=O)c1C]

A solution of (R)—N-[(1R)-1-(2-ethylsulfanyl-3,6-dimethyl-4-oxo-chromen-8-yl)ethyl]-2-methyl-propane-2-sulfinamide (20 g, 52.4 mmol) in THF (200 mL) was treated dropwise with 12M aqueous HCl (8.7 mL, 104.8 mmol) and stirred at rt. After 1 h, the reaction was concentrated, the residue dissolved in water, and extracted with MTBE. The pH of the aqueous phase was adjusted to between 9 and 10 with 1M aqueous NaOH and extracted with EtOAc. The organic layer was washed with saturated aqueous NaCl, dried over MgSO₄, filtered, and concentrated to give the product (12.6 g, 87%) as a white solid. MS ES+ m/z 278 [M+H]⁺.

### Intermediate 23B: tert-Butyl N-[(1R)-1-(2-ethylsulfanyl-3,6-dimethyl-4-oxo-chromen-8-yl)ethyl]carbamate

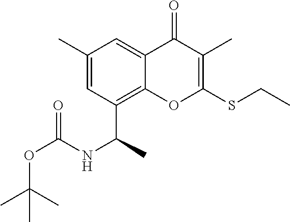 [SMILES: CCSc1oc2c([C@@H](C)NC(=O)OC(C)(C)C)cc(C)cc2c(=O)c1C]

A mixture of 8-[(1R)-1-aminoethyl]-2-ethylsulfanyl-3,6-dimethyl-chromen-4-one (2.5 g, 9.0 mmol) and diisopropylethylamine (2.0 g, 16 mmol) in DCM (40 mL) was treated with di-tert-butyl dicarbonate (3.0 g, 14 mmol) and stirred overnight at rt. Concentrated the reaction and purified the residue by silica gel chromatography eluted with 0 to 100% EtOAc in heptane to give the product (2.5 g, 73%). MS ES+ m/z 378 [M+H]⁺.

### Intermediate 111B: 8-[(1R)-1-[(6-Chloro-3-pyridyl)amino]ethyl]-2-ethylsulfanyl-3,6-dimethyl-chromen-4-one

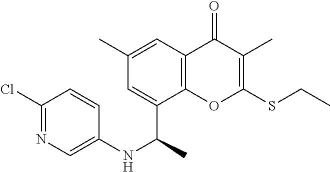 [SMILES: CCSc1oc2c([C@@H](C)Nc3ccc(Cl)nc3)cc(C)cc2c(=O)c1C]

Combined tris(dibenzylideneacetone)dipalladium(0) (0.099 g, 0.11 mmol) and 4,5-bis(diphenylphosphino)-9,9-dimethylxanthene (0.19 g, 0.32 mmol) in toluene (18 mL). Stirred at rt for 10 min. 8-[(1R)-1-Aminoethyl]-2-ethylsulfanyl-3,6-dimethyl-chromen-4-one (0.30 g, 1.1 mmol), 5-bromo-2-chloropyridine (0.21 g, 1.1 mmol), and Cs₂CO₃ (0.71 g, 2.2 mmol) were then added to the mixture, which was flushed with nitrogen. The reaction was sealed and heated to 115° C. overnight. The mixture was filtered over a pad of diatomaceous earth and the solids were rinsed with DCM. The filtrate was concentrated and the residue was purified by reverse phase chromatography eluted with a gradient of 10 to 100% ACN in aqueous NH₄HCO₃ (10 mM, plus 5% MeOH) to give the title compound (0.090 g, 21%). MS ES+ m/z 389 [M+H]⁺.

The following compounds in Table 50 were made in a similar way as described for 8-[(1R)-1-[(6-chloro-3-pyridyl)amino]ethyl]-2-ethylsulfanyl-3,6-dimethyl-chromen-4-one. Various methods were used to purify the compounds, which would be apparent to one skilled in the art.

TABLE 50

| | | | MS ES+ |
|---|---|---|---|
| Interm # | Chemical Name | Structure | m/z |
| 12B | 2-Ethylsulfanyl-3,6- dimethyl-8-[(1R)-1-[(2- methyl-3- pyridyl)amino]ethyl] chromen-4-one | 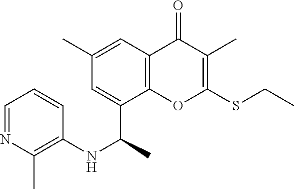 [SMILES: CCSc1oc2c([C@@H](C)Nc3cccnc3C)cc(C)cc2c(=O)c1C] | 369 [M + H]⁺ |
| 13B | 8-[(1R)-1-(2- Bromoanilino)ethyl]- 3,6-dimethyl-2-phenyl- chromen-4-one | 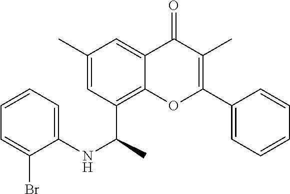 [SMILES: Cc1cc([C@@H](C)Nc2ccccc2Br)c2oc(-c3ccccc3)c(C)c(=O)c2c1] | 450 [M + H]⁺ |
| 24B¹ | 8-[(1R)-1-[(6-Chloro- 2-methyl-3- pyridyl)amino]ethyl]-2- ethylsulfanyl-3,6- dimethyl-chromen-4- one | 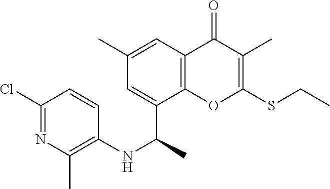 [SMILES: CCSc1oc2c([C@@H](C)Nc3ccc(Cl)nc3C)cc(C)cc2c(=O)c1C] | 403 [M + H]⁺ |
| 25B | 8-[(1R)-1-[(2-Chloro- 3-pyridyl)amino]ethyl]- 2-ethylsulfanyl-3,6- dimethyl-chromen-4- one | 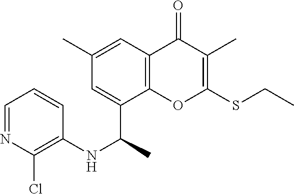 [SMILES: CCSc1oc2c([C@@H](C)Nc3cccnc3Cl)cc(C)cc2c(=O)c1C] | 389 [M + H]⁺ |
| 26B | 8-[(1R)-1-(3,4- Difluoro-2-methyl- anilino)ethyl]-2- ethylsulfanyl-3,6- dimethyl-chromen-4- one | 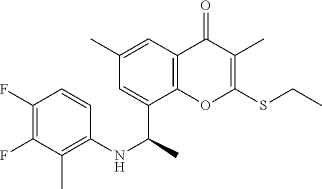 [SMILES: CCSc1oc2c([C@@H](C)Nc3ccc(F)c(F)c3C)cc(C)cc2c(=O)c1C] | 404 [M + H]⁺ |
| 27B | 2-Ethylsulfanyl-3,6- dimethyl-8-[(1R)-1-[[2- methyl-6- (trifluoromethyl)-3- pyridyl]amino]ethyl] chromen-4-one | 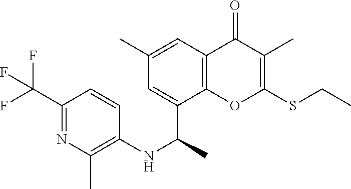 [SMILES: CCSc1oc2c([C@@H](C)Nc3ccc(C(F)(F)F)nc3C)cc(C)cc2c(=O)c1C] | 437 [M + H]⁺ |
| 28B | 8-[(1R)-1-[(6-Chloro- 2-methylsulfanyl-3- pyridyl)amino]ethyl]- 3,6-dimethyl-2-phenyl- chromen-4-one | 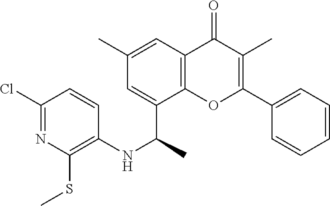 [SMILES: CSc1nc(Cl)ccc1N[C@H](C)c1cc(C)cc2c(=O)c(C)c(-c3ccccc3)oc12] | 449 [M + H]⁺ |

¹Iodo reagent used for this coupling reaction.

### Intermediate 29B: 8-[(1R)-1-[(2,6-Dimethyl-3-pyridyl)amino]ethyl]-2-ethylsulfanyl-3,6-dimethyl-chromen-4-one

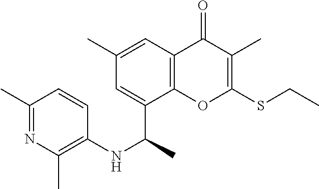 [SMILES: CCSc1oc2c([C@@H](C)Nc3ccc(C)nc3C)cc(C)cc2c(=O)c1C]

A 20 mL pressure vial was charged with 3-bromo-2,6-dimethylpyridine (0.34 g, 1.80 mmol), 8-[(1R)-1-aminoethyl]-2-ethylsulfanyl-3,6-dimethyl-chromen-4-one (0.50 g, 1.80 mmol), cesium carbonate (1.17 g, 3.61 mmol), BINAP (0.34 g, 0.54 mmol), palladium(II) acetate (0.04 g, 0.18 mmol), and 1,4-dioxane (10 mL). Nitrogen gas was bubbled through the mixture for 5 min and the reaction stirred at 90° C. for 15 h. The reaction was allowed to cool to rt, passed through a syringe filter, and purified by reversed phase chromatography eluted with 10 to 100% ACN in 10 mM NH₄HCO₃ with 5% MeOH to give the title compound (0.21 g, 30%) as an off-white foam. MS ES+ m/z 383 [M+H]⁺.

### Intermediate 30B: 2-Ethylsulfanyl-8-[(1R)-1-[(6-methoxy-2-methyl-3-pyridyl)amino]ethyl]-3,6-dimethyl-chromen-4-one

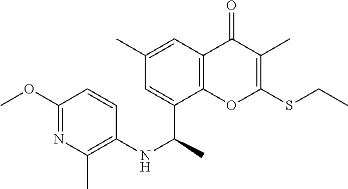 [SMILES: CCSc1oc2c([C@@H](C)Nc3ccc(OC)nc3C)cc(C)cc2c(=O)c1C]

Combined 8-[(1R)-1-aminoethyl]-2-ethylsulfanyl-3,6-dimethyl-chromen-4-one (200 mg, 0.72 mmol), 3-bromo-6-methoxy-2-methylpyridine (189 mg, 0.94 mmol), tris(dibenzylideneacetone)dipalladium(0) (66 mg, 0.07 mmol), dicyclohexyl(2′,4′,6′-triisopropyl-3,6-dimethoxy-[1,1′-biphenyl]-2-yl)phosphane (38.7 mg, 0.07 mmol), and sodium tert-butoxide (139 mg, 1.44 mmol) in 5 mL of 1,4-dioxane. Heated the reaction at 70° C. for 16 h. The reaction was cooled and filtered through diatomaceous earth and the solids washed with EtOAc and DCM. Concentrated the filtrate and purified by silica gel chromatography eluted with 0 to 100% EtOAc in hexane to give the title compound (172 mg, 60%). MS ES+ m/z 399 [M+H]⁺.

### Intermediate 14B: 8-[(1R)-1-Aminoethyl]-2-[6-(difluoromethyl)-2-pyridyl]-3,6-dimethyl-chromen-4-one

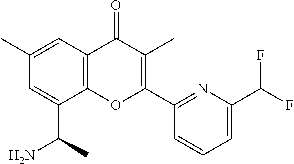 [SMILES: Cc1cc([C@@H](C)N)c2oc(-c3cccc(C(F)F)n3)c(C)c(=O)c2c1]

Combined 8-[(1R)-1-aminoethyl]-2-ethylsulfanyl-3,6-dimethyl-chromen-4-one (5.00 g, 18.0 mmol), 2-(difluoromethyl)-6-(4,4,5,5-tetramethyl-1,3,2-dioxaborolan-2-yl)pyridine (13.8 g, 54.1 mmol), tetrakis(triphenylphosphine)palladium(0) (2.08 g, 1.80 mmol), and copper(I) thiophene-2-carboxylate (5.16 g, 27.0 mmol) in EtOH (150 mL). Degassed the reaction with nitrogen and stirred at 85° C. for 16 h. The mixture was filtered over a pad of diatomaceous earth and the solids were rinsed with DCM. The filtrate was concentrated and the residue was purified by reverse phase chromatography eluted with a gradient of 5 to 100% ACN in aqueous NH₄HCO₃ (10 mM, plus 5% MeOH) to give the title compound (2.00 g, 30%). MS ES+ m/z 345 [M+H]⁺.

The following compounds in Table 51 were made in a similar way as described for 8-[(1R)-1-aminoethyl]-2-[6-(difluoromethyl)-2-pyridyl]-3,6-dimethyl-chromen-4-one. Various methods were used to purify the compounds, which would be apparent to one skilled in the art.

TABLE 51

| | | | MS ES+ |
|---|---|---|---|
| Interm # | Chemical Name | Structure | m/z |
| 15B | 8-[(1R)-1- Aminoethyl]-3,6- dimethyl-2-(3- pyridyl)chromen-4-one | 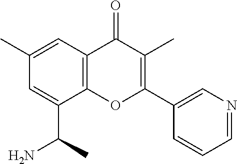 [SMILES: Cc1cc([C@@H](C)N)c2oc(-c3cccnc3)c(C)c(=O)c2c1] | 295 [M + H]⁺ |
| 16B | 8-[(1R)-1-[(6-Chloro- 3-pyridyl)amino]ethyl]- 3,6-dimethyl-2-(1- tetrahydropyran-2- ylindazol-6- yl)chromen-4-one | 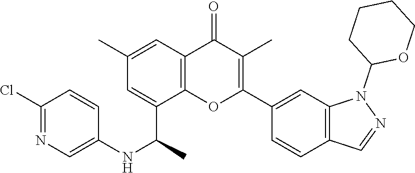 [SMILES: Cc1cc([C@@H](C)Nc2ccc(Cl)nc2)c2oc(-c3ccc4cnn(C5CCCCO5)c4c3)c(C)c(=O)c2c1] | 529 [M + H]⁺ |
| 31B | 8-[(1R)-1- Aminoethyl]-3,6- dimethyl-2-(2- methylindazol-5- yl)chromen-4-one | 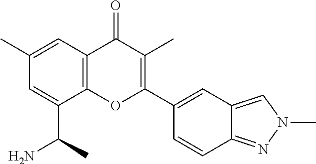 | 348 [M + H]⁺ |
| 32B | 8-[(1R)-1- Aminoethyl]-2-(5- fluoro-3-pyridyl)-3,6- dimethyl-chromen-4- one | 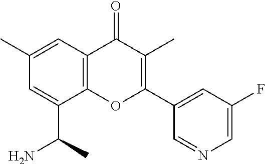 [SMILES: Cc1cc([C@@H](C)N)c2oc(-c3cncc(F)c3)c(C)c(=O)c2c1] | 313 [M + H]⁺ |
| 33B | 8-[(1R)-1- Aminoethyl]-2-(3- fluorophenyl)-3,6- dimethyl-chromen-4- one | 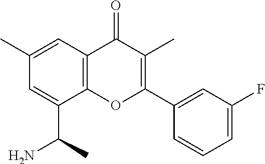 [SMILES: Cc1cc([C@@H](C)N)c2oc(-c3cccc(F)c3)c(C)c(=O)c2c1] | 312 [M + H]⁺ |
| 34B | 8-[(1R)-1-[(6-Chloro- 2-methyl-3- pyridyl)amino]ethyl]- 3,6-dimethyl-2-(1- tetrahydropyran-2- ylindazol-6- yl)chromen-4-one | 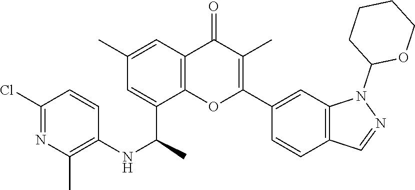 | 529 [M + H]⁺ |
| 35B | 8-[(1R)-1- Aminoethyl]-2-(2,4- difluorophenyl)-3,6- dimethyl-chromen-4- one | 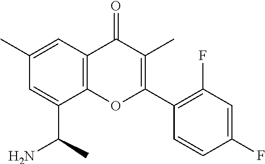 [SMILES: Cc1cc([C@@H](C)N)c2oc(-c3ccc(F)cc3F)c(C)c(=O)c2c1] | — |
| 36B | tert-Butyl N-[(1R)-1- [3,6-dimethyl-2-(1- methyl-6-oxo-3- pyridyl)-4-oxo- chromen-8- yl]ethyl]carbamate | 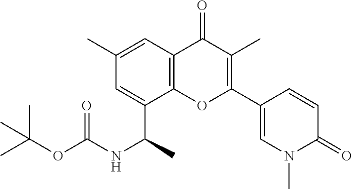 [SMILES: Cc1cc([C@@H](C)NC(=O)OC(C)(C)C)c2oc(-c3ccc(=O)n(C)c3)c(C)c(=O)c2c1] | 425 [M + H]⁺ |
| 37B | tert-Butyl N-[(1R)-1- [3,6-dimethyl-2-(1- methyl-2-oxo-4- pyridyl)-4-oxo- chromen-8- yl]ethyl]carbamate | 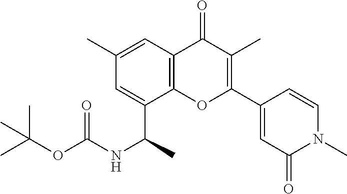 [SMILES: Cc1cc([C@@H](C)NC(=O)OC(C)(C)C)c2oc(-c3ccn(C)c(=O)c3)c(C)c(=O)c2c1] | 425 [M + H]⁺ |

### Intermediate 38B: 5-[8-[(1R)-1-Aminoethyl]-3,6-dimethyl-4-oxo-chromen-2-yl]-1-methyl-pyridin-2-one

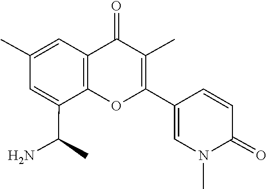 [SMILES: Cc1cc([C@@H](C)N)c2oc(-c3ccc(=O)n(C)c3)c(C)c(=O)c2c1]

tert-Butyl N-[(1R)-1-[3,6-dimethyl-2-(1-methyl-6-oxo-3-pyridyl)-4-oxo-chromen-8-yl]ethyl]carbamate (0.23 g, 0.53 mmol) was dissolved in DCM (5 mL) and treated with TFA (3 mL). The reaction was stirred at 40° C. for 1 h. The reaction was concentrated and purified by reverse phase chromatography eluted with 10 to 100% ACN in 10 mM NH₄HCO₃ with 5% MeOH to give the title compound (0.17 g, 97%). MS ES+ m/z 325 [M+H]⁺.

The following compound in Table 52 was made in a similar way as described for 5-[8-[(1R)-1-aminoethyl]-3,6-dimethyl-4-oxo-chromen-2-yl]-1-methyl-pyridin-2-one.

TABLE 52

| | | | MS ES+ |
|---|---|---|---|
| Interm # | Chemical Name | Structure | m/z |
| 39B | 4-[8-[(1R)-1- Aminoethyl]-3,6- dimethyl-4-oxo- chromen-2-yl]-1- methyl-pyridin- 2-one | 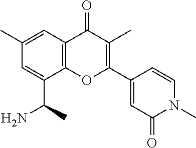 | 325 [M + H]⁺ |

### Intermediate 40B: tert-Butyl 3-[4-[8-[(1R)-1-[(6-chloro-3-pyridyl)amino]ethyl]-3,6-dimethyl-4-oxo-chromen-2-yl]phenyl]azetidine-1-carboxylate

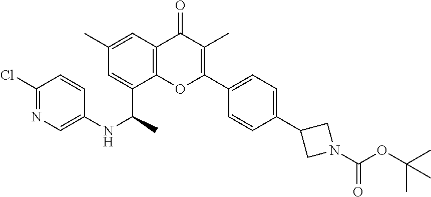 [SMILES: Cc1cc([C@@H](C)Nc2ccc(Cl)nc2)c2oc(-c3ccc(C4CN(C(=O)OC(C)(C)C)C4)cc3)c(C)c(=O)c2c1]

8-[(1R)-1-[(6-Chloro-3-pyridyl)amino]ethyl]-2-ethylsulfanyl-3,6-dimethyl-chromen-4-one (0.20 g, 0.51 mmol) and copper(I) thiophene-2-carboxylate (0.15 g, 0.77 mmol) were combined in EtOH (10 mL) in a vial and degassed for 5 min with nitrogen gas. Tetrakis(triphenylphosphine)palladium(0) (0.12 g, 0.10 mmol) and tert-butyl 3-[4-(4,4,5,5-tetramethyl-1,3,2-dioxaborolan-2-yl)phenyl]azetidine-1-carboxylate (0.22 g, 0.62 mmol) were then added, the vial sealed, and the reaction stirred at 40° C. for 4 h. After cooling to rt, the reaction was filtered, concentrated, and purified by silica gel chromatography eluted with 0 to 100% EtOAc in heptane to give the title compound (0.18 g, 59%). MS ES+ m/z 560 [M+H]⁺.

### Intermediate 17B: 3,6-Dimethyl-2-phenyl-8-[(1R)-1-(2-vinylanilino)ethyl]chromen-4-one

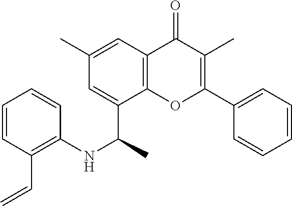 [SMILES: C=Cc1ccccc1N[C@H](C)c1cc(C)cc2c(=O)c(C)c(-c3ccccc3)oc12]

8-[(1R)-1-(2-Bromoanilino)ethyl]-3,6-dimethyl-2-phenyl-chromen-4-one (0.10 g, 0.22 mmol), tributyl(vinyl)stannane (0.085 g, 0.27 mmol), and tetrakis(triphenylphosphine)palladium(0) (0.026 g, 0.022 mmol) were combined in toluene (2 mL). The reaction was degassed with argon and stirred at 120° C. for 2 h. Purified by silica gel chromatography eluted with a gradient of 0 to 100% EtOAc in hexanes to give the title compound (0.082 g, 93%). MS ES+ m/z 396 [M+H]⁺.

### Intermediate 41B: Trimethyl-(1-methylpyrazolo[3,4-b]pyridin-6-yl)stannane

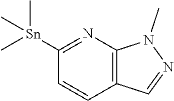

A stirred solution of 6-chloro-1-methyl-pyrazolo[3,4-b]pyridine (1.1 g, 6.56 mmol) and hexamethyldistannane (2.58 g, 7.88 mmol) in 10 mL of 1,4-dioxane was treated with tetrakis(triphenylphosphine)palladium(0) (0.76 g, 0.66 mmol) in portions at rt under nitrogen. The resulting mixture was then stirred at 100° C. for 2 h. The reaction was allowed to cool to rt and quenched with 100 mL of saturated aqueous cesium fluoride. Extracted with EtOAc (2×100 mL). The combined organics were washed with saturated aqueous NaCl, dried over anhydrous Na₂SO₄, filtered, and concentrated to give the title compound (2.1 g, crude) as a yellow oil. MS ES+ m/z 298 [M+H]⁺.

The following compound in Table 53 was made in a similar way as described for trimethyl-(1-methylpyrazolo[3,4-b]pyridin-6-yl)stannane. Various methods were used to purify the compound, which would be apparent to one skilled in the art.

TABLE 53

| | | | MS ES+ |
|---|---|---|---|
| Interm # | Chemical Name | Structure | m/z |
| 380C | Trimethyl-(1- methylpyrazolo[4,3- b]pyridin-5-yl)stannane | 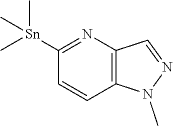 | 298 [M + H]⁺ |

### Intermediate 381C: 8-[(1R)-1-[(6-Chloro-2-methylsulfinyl-3-pyridyl)amino]ethyl]-3,6-dimethyl-2-phenyl-chromen-4-one

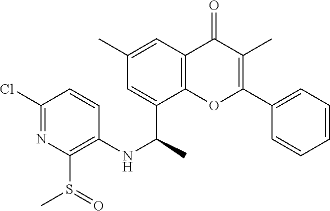 [SMILES: Cc1cc([C@@H](C)Nc2ccc(Cl)nc2S(C)=O)c2oc(-c3ccccc3)c(C)c(=O)c2c1]

A solution of 8-[(1R)-1-[(6-chloro-2-methylsulfanyl-3-pyridyl)amino]ethyl]-3,6-dimethyl-2-phenyl-chromen-4-one (150 mg, 0.33 mmol) in DCM (3 mL) was treated dropwise with mCPBA (51.66 mg, 0.30 mmol) at rt. The reaction was stirred at rt for 4 h and then concentrated under reduced pressure. The residue was purified by silica gel chromatography eluted with 25% EtOAc in petroleum ether to give the title compound (54 mg, 35%) as a white solid. ES/MS m/z 467 (M+H).

### Example 1A: 8-[(1R)-1-[4-Chloro-2-(difluoromethyl)anilino]ethyl]-3,6-dimethyl-2-phenyl-chromen-4-one

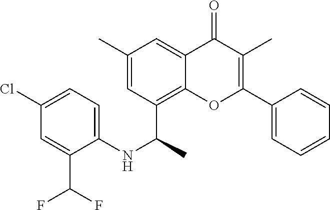 [SMILES: Cc1cc([C@@H](C)Nc2ccc(Cl)cc2C(F)F)c2oc(-c3ccccc3)c(C)c(=O)c2c1]

A mixture of tris(dibenzylideneacetone)dipalladium(0) (0.031 g, 0.034 mmol) and 4,5-bis(diphenylphosphino)-9,9-dimethylxanthene (0.059 g, 0.102 mmol) in toluene (7 mL) was stirred at rt for 10 min. 8-[(1R)-1-Aminoethyl]-3,6-dimethyl-2-phenyl-chromen-4-one (0.100 g, 0.341 mmol), 4-bromo-3-(difluoromethyl)chlorobenzene (0.082 g, 0.341 mmol), and Cs₂CO₃ (0.222 g, 0.682 mmol) were then added to the mixture, which was flushed with nitrogen. The reaction was sealed and heated to 115° C. overnight. The mixture was filtered over a pad of diatomaceous earth and the solids were rinsed with DCM. The filtrate was concentrated and the residue was purified by reverse phase chromatography eluted with a gradient of 10 to 100% ACN in aqueous NH₄HCO₃ (10 mM, plus 5% MeOH) to give the title compound (0.122 g, 79%). MS ES+ m/z 454 [M+H]⁺.

The following compounds in Table 54 were made in a similar way as described for 8-[(1R)-1-[4-chloro-2-(difluoromethyl)anilino]ethyl]-3,6-dimethyl-2-phenyl-chromen-4-one.

TABLE 54

| Example | | | MS ES+ |
|---|---|---|---|
| # | Chemical Name | Structure | m/z |
| 2A | 2-[[(1R)-1-(3,6- Dimethyl-4-oxo-2- phenyl-chromen-8- yl)ethyl]amino] benzonitrile | 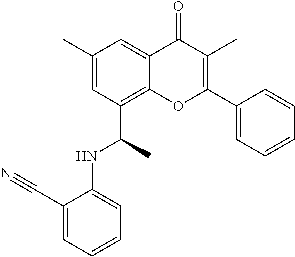 [SMILES: Cc1cc([C@@H](C)Nc2ccccc2C#N)c2oc(-c3ccccc3)c(C)c(=O)c2c1] | 395 [M + H]⁺ |
| 3A | 7-[(1R)-1-(3,6- Dimethyl-4-oxo-2- phenyl-chromen-8- yl)ethyl]amino]isoindolin- 1-one | 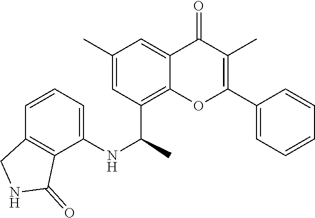 [SMILES: Cc1cc([C@@H](C)Nc2cccc3c2C(=O)NC3)c2oc(-c3ccccc3)c(C)c(=O)c2c1] | 425 [M + H]⁺ |
| 4A | 8-[(1R)-1-[3- (Difluoromethyl)anilino] ethyl]-3,6-dimethyl- 2-phenyl-chromen-4- one | 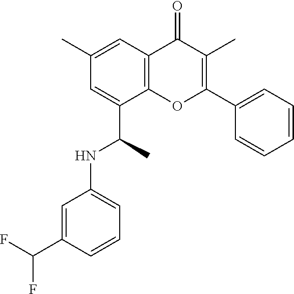 [SMILES: Cc1cc([C@@H](C)Nc2cccc(C(F)F)c2)c2oc(-c3ccccc3)c(C)c(=O)c2c1] | 420 [M + H]⁺ |
| 5A | Methyl 3-[(1R)-1-(3,6- dimethyl-4-oxo-2- phenyl-chromen-8- yl)ethyl]amino]thiophene- 2-carboxylate | 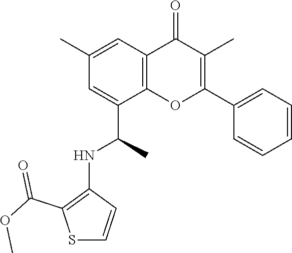 [SMILES: COC(=O)c1sccc1N[C@H](C)c1cc(C)cc2c(=O)c(C)c(-c3ccccc3)oc12] | 434 [M + H]⁺ |
| 6A | 8-[(1R)-1-[(2- Methoxy-3- pyridyl)amino]ethyl]- 3,6-dimethyl-2-phenyl- chromen-4-one | 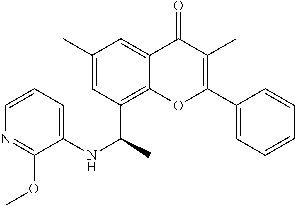 [SMILES: COc1ncccc1N[C@H](C)c1cc(C)cc2c(=O)c(C)c(-c3ccccc3)oc12] | 401 [M + H]+ |
| 104A | 8-[(1R)-1-[2- (Difluoromethyl)-3- pyridyl]amino]ethyl]- 3,6-dimethyl-2-(2- methylindazol-5- yl)chromen-4-one | 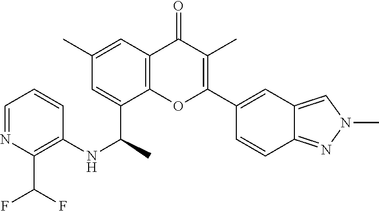 [SMILES: Cc1cc([C@@H](C)Nc2cccnc2C(F)F)c2oc(-c3ccc4nn(C)cc4c3)c(C)c(=O)c2c1] | 475 [M + H]⁺ |
| 105A | 3,6-Dimethyl-2-(2- methylindazol-5-yl)-8- [(1R)-1-[[2- (trifluoromethyl)-3- pyridyl]amino]ethyl] chromen-4-one | 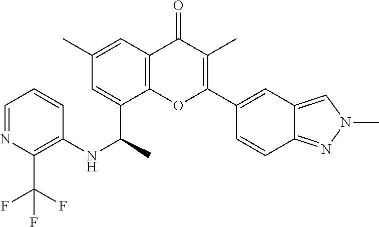 [SMILES: Cc1cc([C@@H](C)Nc2cccnc2C(F)(F)F)c2oc(-c3ccc4nn(C)cc4c3)c(C)c(=O)c2c1] | 493 [M + H]⁺ |
| 106A¹ | 8-[(1R)-1-[[6-Chloro- 2-(trifluoromethyl)-3- pyridyl]amino]ethyl]- 3,6-dimethyl-2-(3-994 pyridyl)chromen-4-one | 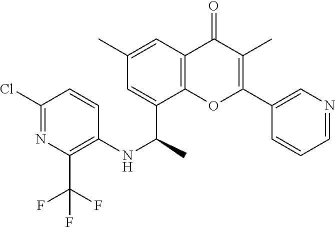 [SMILES: Cc1cc([C@@H](C)Nc2ccc(Cl)nc2C(F)(F)F)c2oc(-c3cccnc3)c(C)c(=O)c2c1] | 474 [M + H]⁺ |
| 107A¹ | 8-[(1R)-1-[[6-Chloro- 2-(trifluoromethyl)-3- pyridyl]amino]ethyl]- 3,6-dimethyl-2-(2- methylindazol-5- yl)chromen-4-one | 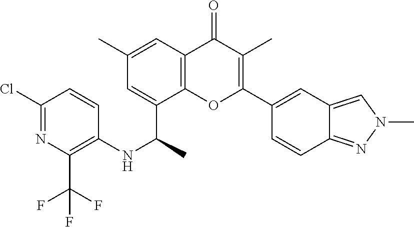 [SMILES: Cc1cc([C@@H](C)Nc2ccc(Cl)nc2C(F)(F)F)c2oc(-c3ccc4nn(C)cc4c3)c(C)c(=O)c2c1] | 527 [M + H]⁺ |
| 108A | 8-[(1R)-1-[[2- (Difluoromethyl)-3- pyridyl]amino]ethyl]- 3,6-dimethyl-2-phenyl- chromen-4-one | 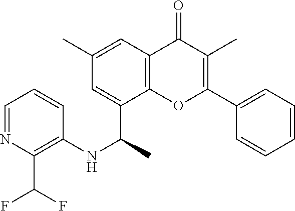 [SMILES: Cc1cc([C@@H](C)Nc2cccnc2C(F)F)c2oc(-c3ccccc3)c(C)c(=O)c2c1] | 421 [M + H]⁺ |
| 109A | 3,6-Dimethyl-2-(3- pyridyl)-8-[(1R)-1-[[2- (trifluoromethyl)-3- pyridyl]amino]ethyl] chromen-4-one | 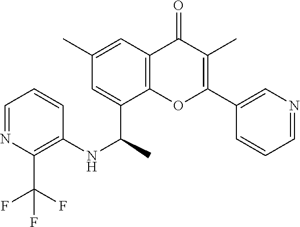 [SMILES: Cc1cc([C@@H](C)Nc2cccnc2C(F)(F)F)c2oc(-c3cccnc3)c(C)c(=O)c2c1] | 440 [M + H]⁺ |
| 110A | 5-Chloro-2-[[(1R)-1- [3,6-dimethyl-4-oxo-2- (3-pyridyl)chromen-8- yl]ethyl]amino]benzonitrile | 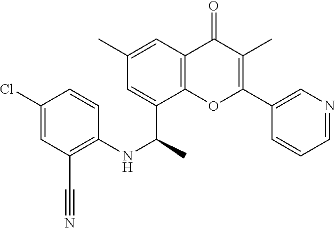 [SMILES: Cc1cc([C@@H](C)Nc2ccc(Cl)cc2C#N)c2oc(-c3cccnc3)c(C)c(=O)c2c1] | 430 [M + H]⁺ |
| 111A | 8-[(1R)-1-[[6-Chloro- 2-(difluoromethyl)-3- pyridyl]amino]ethyl]- 3,6-dimethyl-2-(2- methylindazol-5- yl)chromen-4-one | 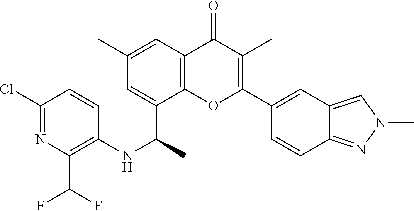 [SMILES: Cc1cc([C@@H](C)Nc2ccc(Cl)nc2C(F)F)c2oc(-c3ccc4nn(C)cc4c3)c(C)c(=O)c2c1] | 509 [M + H]⁺ |

¹1,4-Dioxane used as solvent.

### Example 112A: 7-[[(1R)-1-[3,6-Dimethyl-4-oxo-2-(3-pyridyl)chromen-8-yl]ethyl]amino]isoindolin-1-one

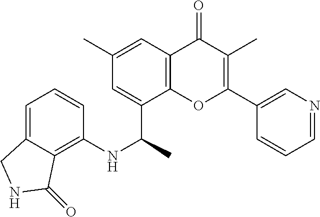 [SMILES: Cc1cc([C@@H](C)Nc2cccc3c2C(=O)NC3)c2oc(-c3cccnc3)c(C)c(=O)c2c1]

A solution of tert-butyl 7-[[(1R)-1-[3,6-dimethyl-4-oxo-2-(3-pyridyl)chromen-8-yl]ethyl]amino]-1-oxo-isoindoline-2-carboxylate (0.13 g, 0.25 mmol) in 2 mL of DCM was treated with 1 mL of TFA and allowed to stir at rt for 1 h. The reaction mixture was concentrated and the residue purified by reverse phase chromatography eluted with 0% to 100% ACN in water (with 0.1% formic acid). Fractions containing the product were combined, washed with saturated aqueous sodium chloride, extracted with IPA/CHCl₃ (1:3), the organics collected, dried over MgSO₄, filtered, and concentrated. The residue was recrystallized from a mixture of DCM and hexane to obtain the title compound (91.5 mg, 86%) as a white solid. MS ES+ m/z 426 [M+H]⁺.

The following compounds in Table 55 were made in a similar way as described for 7-[[(1R)-1-[3,6-dimethyl-4-oxo-2-(3-pyridyl)chromen-8-yl]ethyl]amino]isoindolin-1-one. Various methods were used to purify the compounds, which would be apparent to one skilled in the art.

TABLE 55

| Example | | | MS ES+ |
|---|---|---|---|
| # | Chemical Name | Structure | m/z |
| 1C | 8-[(1R)-1-(1H-Indol-4- ylamino)ethyl]-3,6- dimethyl-2-(3- pyridyl)chromen-4-one | 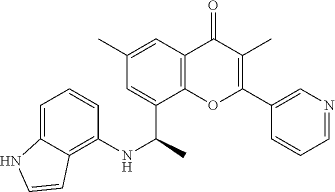 [SMILES: Cc1cc([C@@H](C)Nc2cccc3[nH]ccc23)c2oc(-c3cccnc3)c(C)c(=O)c2c1] | 410 [M + H]⁺ |
| 20 | 3,6-Dimethyl-8-[(1R)- 1-[(7-methyl-1H-indol- 6-yl)amino]ethyl]-2-(3- pyridyl)chromen-4-one | 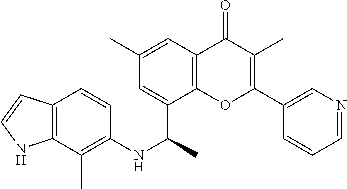 [SMILES: Cc1cc([C@@H](C)Nc2ccc3cc[nH]c3c2C)c2oc(-c3cccnc3)c(C)c(=O)c2c1] | 424 [M + H]⁺ |
| 30 | 6-Chloro-3-[[(1R)-1- [3,6-dimethyl-4-oxo-2- (4,5,6,7- tetrahydropyrazolo[1,5- alpyrazin-3- yl)chromen-8- yl]ethyl]amino]-N- methylsulfonyl- pyridine-2- carboxamide | 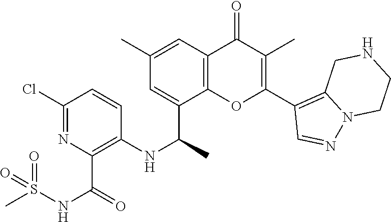 [SMILES: Cc1cc([C@@H](C)Nc2ccc(Cl)nc2C(=O)NS(C)(=O)=O)c2oc(-c3cnn4c3CNCC4)c(C)c(=O)c2c1] | 571 [M + H]⁺ |

### Example 7A: 3,6-Dimethyl-2-(3-pyridyl)-8-[(1R)-1-[[2-(2H-tetrazol-5-yl)-3-pyridyl]amino]ethyl]chromen-4-one

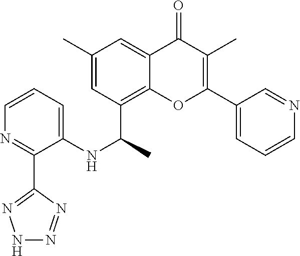 [SMILES: Cc1cc([C@@H](C)Nc2cccnc2-c2nn[nH]n2)c2oc(-c3cccnc3)c(C)c(=O)c2c1]

A mixture of 3-[[(1R)-1-[3,6-dimethyl-4-oxo-2-(3-pyridyl)chromen-8-yl]ethyl]amino]pyridine-2-carbonitrile (0.040 g, 0.10 mmol), sodium azide (0.033 g, 0.51 mmol) and ammonium chloride (0.027 g, 0.50 mmol) in DMF (1 mL) was heated at 130° C. overnight. The reaction was concentrated and purified by reverse phase chromatography eluted with a gradient of 0 to 100% ACN in H₂O+0.1% formic acid. Fractions containing the desired product were combined, washed with saturated aqueous sodium chloride, and extracted with IPA:CHCl₃ (1:3). The combined extracts were dried over MgSO₄, filtered, and concentrated. The material was further purified by reverse phase chromatography eluted with a gradient of 0 to 100% ACN in aqueous NH₄HCO₃ (10 mM, plus 5% MeOH). Fractions containing the desired product were combined, washed with saturated aqueous NH₄Cl, and extracted with IPA:CHCl₃ (1:3). The combined extracts were dried over MgSO₄, filtered, and concentrated to give the title compound (0.122 g, 79%) as a white solid. MS ES+ m/z 440 [M+H]⁺.

The following compounds in Table 56 were made in a similar way as described for 3,6-dimethyl-2-phenyl-8-[(1R)-1-[2-(2H-tetrazol-5-yl)anilino]ethyl]chromen-4-one. Various methods were used to purify the compounds, which would be apparent to one skilled in the art.

TABLE 56

| Example | | | MS ES+ |
|---|---|---|---|
| # | Chemical Name | Structure | m/z |
| 8A | 2-(3,4-Difluorophenyl)- 3,6-dimethyl-8-[(1R)- 1-[2-(2H-tetrazol-5- yl)-3- pyridyl]amino]ethyl] chromen-4-one | 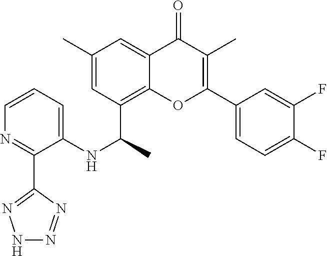 [SMILES: Cc1cc([C@@H](C)Nc2cccnc2-c2nn[nH]n2)c2oc(-c3ccc(F)c(F)c3)c(C)c(=O)c2c1] | 475 [M + H]⁺ |
| 9A | 3,6-Dimethyl-2-phenyl- 8-[(1R)-1-[2-(2H- tetrazol-5- yl)anilino]ethyl]chromen- 4-one | 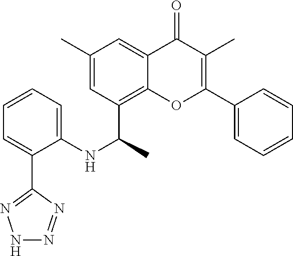 [SMILES: Cc1cc([C@@H](C)Nc2ccccc2-c2nn[nH]n2)c2oc(-c3ccccc3)c(C)c(=O)c2c1] | 438 [M + H]⁺ |
| 10A | 3-[3,6-Dimethyl-4-oxo- 8-[(1R)-1-[2-(2H- tetrazol-5- yl)anilino]ethyl]chromen- 2-yl]benzonitrile | 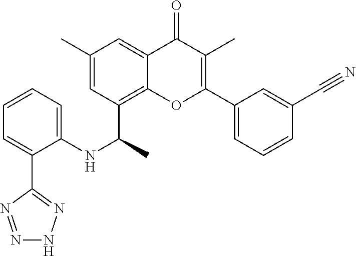 [SMILES: Cc1cc([C@@H](C)Nc2ccccc2-c2nn[nH]n2)c2oc(-c3cccc(C#N)c3)c(C)c(=O)c2c1] | 463 [M + H]⁺ |
| 11A | 3,6-Dimethyl-2-(2- pyridyl)-8-[(1R)-1-[2- (2H-tetrazol-5- yl)anilino]ethyl]chromen- 4-one | 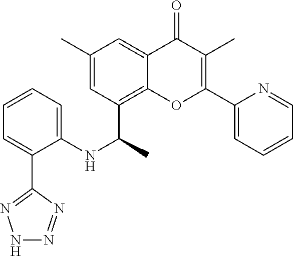 [SMILES: Cc1cc([C@@H](C)Nc2ccccc2-c2nn[nH]n2)c2oc(-c3ccccn3)c(C)c(=O)c2c1] | 439 [M + H]⁺ |
| 12A | 3,6-Dimethyl-2-(3- pyridyl)-8-[(1R)-1-[2- (2H-tetrazol-5- yl)anilino]ethyl]chromen- 4-one | 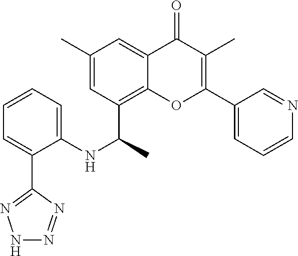 [SMILES: Cc1cc([C@@H](C)Nc2ccccc2-c2nn[nH]n2)c2oc(-c3cccnc3)c(C)c(=O)c2c1] | 439 [M + H]⁺ |
| 13A | 3,6-Dimethyl-2-(2- methylindazol-5-yl)-8- [(1R)-1-[2-(2H- tetrazol-5- yl)anilino]ethyl]chromen- 4-one | 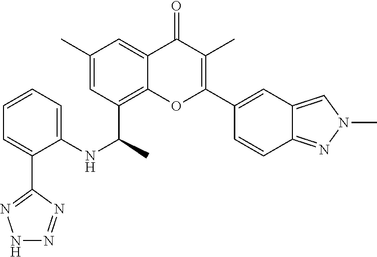 [SMILES: Cc1cc([C@@H](C)Nc2ccccc2-c2nn[nH]n2)c2oc(-c3ccc4nn(C)cc4c3)c(C)c(=O)c2c1] | 492 [M + H]⁺ |
| 14A | 2-(3-Fluorophenyl)- 3,6-dimethyl-8-[(1R)- 1-[2-(2H-tetrazol-5- yl)anilino]ethyl]chromen- 4-one | 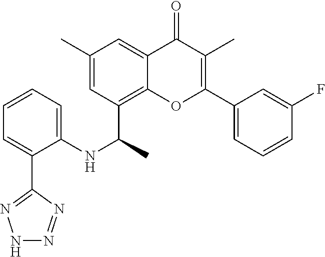 [SMILES: Cc1cc([C@@H](C)Nc2ccccc2-c2nn[nH]n2)c2oc(-c3cccc(F)c3)c(C)c(=O)c2c1] | 456 [M + H]⁺ |
| 15A | 2-(2-Fluorophenyl)- 3,6-dimethyl-8-[(1R)- 1-[2-(2H-tetrazol-5- yl)anilino]ethyl]chromen- 4-one | 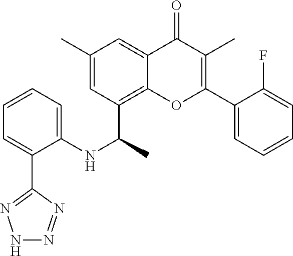 [SMILES: Cc1cc([C@@H](C)Nc2ccccc2-c2nn[nH]n2)c2oc(-c3ccccc3F)c(C)c(=O)c2c1] | 456 [M + H]⁺ |
| 16A | 3-Methyl-2-phenyl-8- [(1R)-1-[2-(2H- tetrazol-5-yl)-3- pyridyl]amino]ethyl]-6- (trifluoromethyl)chromen- 4-one | 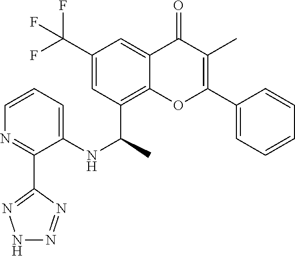 [SMILES: Cc1c(-c2ccccc2)oc2c([C@@H](C)Nc3cccnc3-c3nn[nH]n3)cc(C(F)(F)F)cc2c1=O] | 493 [M + H]⁺ |
| 17A | 3,6-Dimethyl-2-phenyl- 8-[(1R)-1-[[3-(2H- tetrazol-5-yl)-2- pyridyl]amino]ethyl] chromen-4-one | 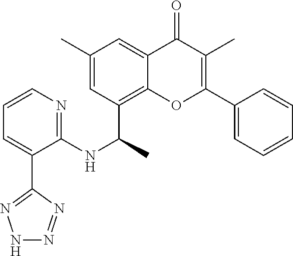 [SMILES: Cc1cc([C@@H](C)Nc2ncccc2-c2nn[nH]n2)c2oc(-c3ccccc3)c(C)c(=O)c2c1] | 439 [M + H]⁺ |
| 18A | 3,6-Dimethyl-8-[(1R)- 1-[6-methyl-2-(2H- tetrazol-5-yl)-3- pyridyl]amino]ethyl]-2- phenyl-chromen-4-one | 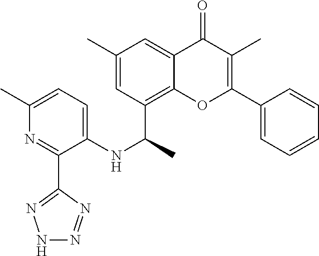 [SMILES: Cc1cc([C@@H](C)Nc2ccc(C)nc2-c2nn[nH]n2)c2oc(-c3ccccc3)c(C)c(=O)c2c1] | 453 [M + H]⁺ |
| 19A | 8-[(1R)-1-[[6- Methoxy-2-(2H- tetrazol-5-yl)-3- pyridyl]amino]ethyl]- 3,6-dimethyl-2-phenyl- chromen-4-one | 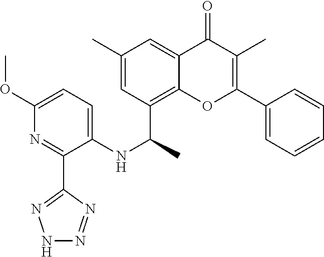 [SMILES: COc1ccc(N[C@H](C)c2cc(C)cc3c(=O)c(C)c(-c4ccccc4)oc23)c(-c2nn[nH]n2)n1] | 469 [M + H]⁺ |
| 20A | 8-[(1R)-1-[[6-Chloro- 2-(2H-tetrazol-5-yl)-3- pyridyl]amino]ethyl]- 3,6-dimethyl-2-phenyl- chromen-4-one | 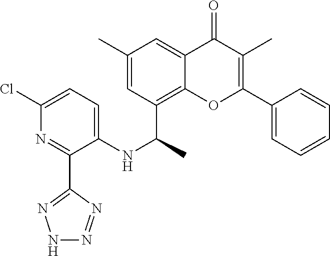 [SMILES: Cc1cc([C@@H](C)Nc2ccc(Cl)nc2-c2nn[nH]n2)c2oc(-c3ccccc3)c(C)c(=O)c2c1] | 473 [M + H]⁺ |
| 21A | 3,6-Dimethyl-2-phenyl- 8-[(1R)-1-[[2-(2H- tetrazol-5-yl)-3- pyridyl]amino]ethyl] chromen-4-one | 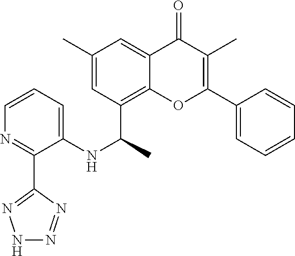 [SMILES: Cc1cc([C@@H](C)Nc2cccnc2-c2nn[nH]n2)c2oc(-c3ccccc3)c(C)c(=O)c2c1] | 439 [M + H]⁺ |
| 22A | 3,6-Dimethyl-2-(1- methylpyrazol-4-yl)-8- [(1R)-1-[2-(2H- tetrazol-5- yl)anilino]ethyl]chromen- 4-one | 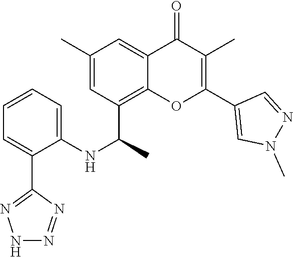 [SMILES: Cc1cc([C@@H](C)Nc2ccccc2-c2nn[nH]n2)c2oc(-c3cnn(C)c3)c(C)c(=O)c2c1] | |
| 113A | 3,6-Dimethyl-2-(2- methylindazol-5-yl)-8- [(1R)-1-[[6-methyl-2- (2H-tetrazol-5-yl)-3- pyridyl]amino]ethyl] chromen-4-one | 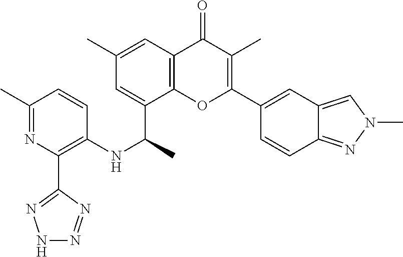 | 507 [M + H]⁺ |
| 114A | 2-(2-Fluorophenyl)-8- [(1R)-1-[3-fluoro-2- (2H-tetrazol-5- yl)anilino]ethyl]-3,6- dimethyl-chromen-4- one | 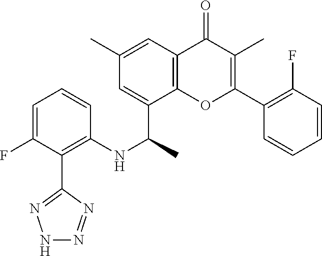 [SMILES: Cc1cc([C@@H](C)Nc2cccc(F)c2-c2nn[nH]n2)c2oc(-c3ccccc3F)c(C)c(=O)c2c1] | 474 [M + H]⁺ |
| 115A | 2-(2-Fluorophenyl)-8- [(1R)-1-[4-fluoro-2- (2H-tetrazol-5- yl)anilino]ethyl]-3,6- dimethyl-chromen-4- one | 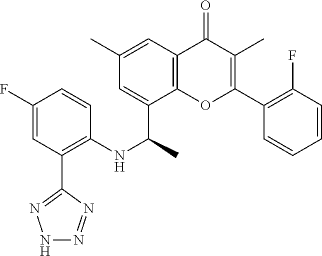 [SMILES: Cc1cc([C@@H](C)Nc2ccc(F)cc2-c2nn[nH]n2)c2oc(-c3ccccc3F)c(C)c(=O)c2c1] | 474 [M + H]⁺ |
| 116A | 2-(2-Fluorophenyl)- 3,6-dimethyl-8-[(1R)- 1-[6-methyl-2-(2H- tetrazol-5-yl)-3- pyridyl]amino]ethyl] chromen-4-one | 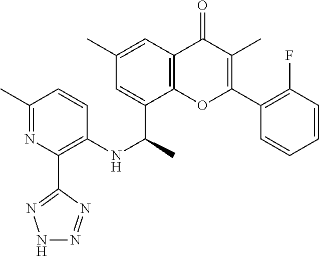 [SMILES: Cc1cc([C@@H](C)Nc2ccc(C)nc2-c2nn[nH]n2)c2oc(-c3ccccc3F)c(C)c(=O)c2c1] | 471 [M + H]⁺ |
| 117A | 2-[6-(Difluoromethyl)- 2-pyridyl]-3,6- dimethyl-8-[(1R)-1-[[6- methyl-2-(2H-tetrazol- 5-yl)-3- pyridyl]amino]ethyl] chromen-4-one | 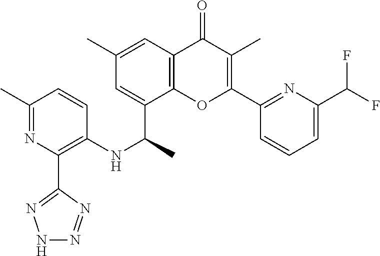 [SMILES: Cc1cc([C@@H](C)Nc2ccc(C)nc2-c2nn[nH]n2)c2oc(-c3cccc(C(F)F)n3)c(C)c(=O)c2c1] | 504 [M + H]⁺ |
| 118A | 2-(2-Fluorophenyl)-8- [(1R)-1-[5-fluoro-2- (2H-tetrazol-5- yl)anilino]ethyl]-3,6- dimethyl-chromen-4- one | 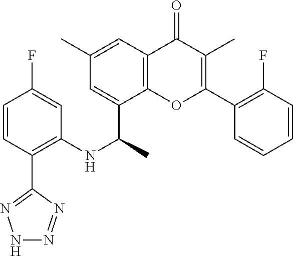 [SMILES: Cc1cc([C@@H](C)Nc2cc(F)ccc2-c2nn[nH]n2)c2oc(-c3ccccc3F)c(C)c(=O)c2c1] | 474 [M + H]⁺ |

### Example 23A and Example 24A: 3,6-Dimethyl-2-phenyl-8-[1-[2-(tetrazol-1-yl)anilino]ethyl]chromen-4-one, Isomer 1 and Isomer 2

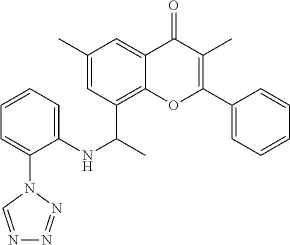 [SMILES: Cc1cc(C(C)Nc2ccccc2-n2cnnn2)c2oc(-c3ccccc3)c(C)c(=O)c2c1]

To a solution of 8-(1-chloroethyl)-3,6-dimethyl-2-phenyl-chromen-4-one (0.200 g, 0.639 mmol) and 2-(1H-tetrazol-1-yl)aniline (0.206 g, 1.28 mmol) in IPA (2 mL) was added triethylamine (0.27 mL, 1.92 mmol). The reaction was heated in a sealed vessel at 90° C. for 18 h, then additional 2-(1H-tetrazol-1-yl)aniline (0.206 g, 1.28 mmol) and triethylamine (0.27 mL, 1.92 mmol) were added and heating continued at 90° C. for 24 h. The reaction was concentrated and the residue was purified by silica gel chromatography eluted with a gradient of 0% to 100% EtOAc in hexanes to give crude racemic product, which was recrystallized from a mixture of DCM and hexanes. The racemic mixture was separated by chiral SFC [Chiralpak IH, 20×250 mm; 35% EtOH (0.5% DMEA) in CO₂] to give the title compounds (Isomer 1, first-eluting; 0.026 g, >98% ee, 90%) and (Isomer 2; 0.034 g, 9700 ee, 120%). MS ES+ m/z 438 [M+H]⁺.

The following compounds in Table 57 were made in a similar way as described for 3,6-dimethyl-2-phenyl-8-[1-[2-(tetrazol-1-yl)anilino]ethyl]chromen-4-one. Various methods were used to purify the compounds, which would be apparent to one skilled in the art.

TABLE 57

| Example | | | MS ES+ |
|---|---|---|---|
| # | Chemical Name | Structure | m/z |
| 25A | 4-[1-(3,6-Dimethyl-4- oxo-2-phenyl-chromen- 8- yl)ethylamino]indoline- 2,3-dione 2,2,2- trifluoroacetic acid | 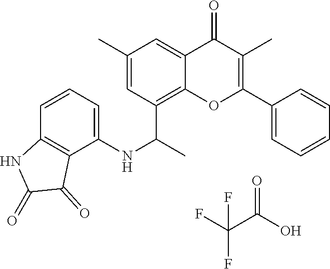 | 439 [M + H]⁺ |
| 26A | 4-[1-(3,6-Dimethyl-4- oxo-2-phenyl-chromen- 8-yl)ethylamino]-1,1- dioxo-1,2- benzothiazol-3-one 2,2,2-trifluoroacetic acid | 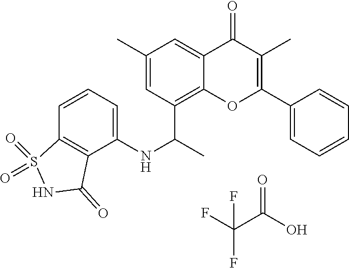 [SMILES: Cc1cc(C(C)Nc2cccc3c2C(=O)NS3(=O)=O)c2oc(-c3ccccc3)c(C)c(=O)c2c1 ; O=C(O)C(F)(F)F] | 475 [M + H]⁺ |
| 27A ª | 4-[1-(3,6-Dimethyl-4- oxo-2-phenyl-chromen- 8- yl)ethylaminolindoline- 2,3-dione, Isomer 1 | 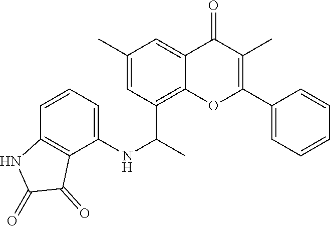 [SMILES: Cc1cc(C(C)Nc2cccc3c2C(=O)C(=O)N3)c2oc(-c3ccccc3)c(C)c(=O)c2c1] | 439 [M + H]⁺ |
| 28A ª | 4-[1-(3,6-Dimethyl-4- oxo-2-phenyl-chromen- 8- yl)ethylamino]indoline- 2,3-dione, Isomer 2 | 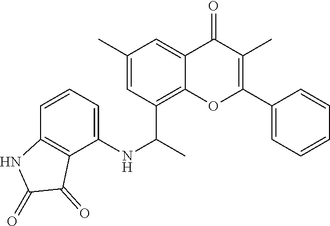 [SMILES: Cc1cc(C(C)Nc2cccc3c2C(=O)C(=O)N3)c2oc(-c3ccccc3)c(C)c(=O)c2c1] | 439 [M + H]⁺ |
| 29A ^{b} | 4-[1-(3,6-Dimethyl-4- oxo-2-phenyl-chromen- 8-yl)ethylamino]-1,1- dioxo-1,2- benzothiazol-3-one, Isomer 1 | 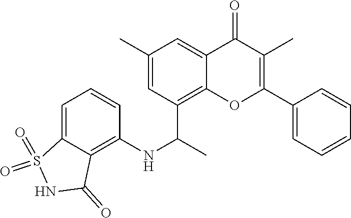 [SMILES: Cc1cc(C(C)Nc2cccc3c2C(=O)NS3(=O)=O)c2oc(-c3ccccc3)c(C)c(=O)c2c1] | 475 [M + H]⁺ |
| 30A ^{b} | 4-[1-(3,6-Dimethyl-4- oxo-2-phenyl-chromen- 8-yl)ethylamino]-1,1- dioxo-1,2- benzothiazol-3-one, Isomer 2 | 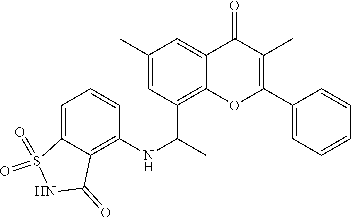 [SMILES: Cc1cc(C(C)Nc2cccc3c2C(=O)NS3(=O)=O)c2oc(-c3ccccc3)c(C)c(=O)c2c1] | 475 [M + H]⁺ |
| 31A | 3,6-Dimethyl-8-[(1R)- 1-[(3-oxo-1H- isobenzofuran-4- yl)amino]ethyl]-2- phenyl-chromen-4-one | 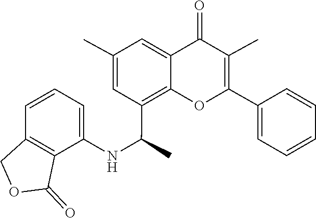 [SMILES: Cc1cc([C@@H](C)Nc2cccc3c2C(=O)OC3)c2oc(-c3ccccc3)c(C)c(=O)c2c1] | 426 [M + H]⁺ |
| 32A | 2-[1-(3,6-Dimethyl-4- oxo-2-phenyl-chromen- 8- yl)ethylamino]benzene sulfonamide | 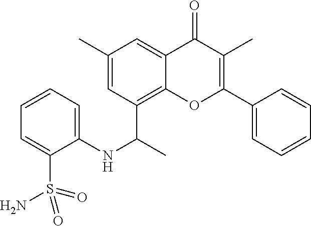 [SMILES: Cc1cc(C(C)Nc2ccccc2S(N)(=O)=O)c2oc(-c3ccccc3)c(C)c(=O)c2c1] | 449 [M + H]⁺ |
| 33A ^{c} | 2-[1-(3,6-Dimethyl-4- oxo-2-phenyl-chromen- 8- yl)ethylamino]benzene sulfonamide, Isomer 1 | 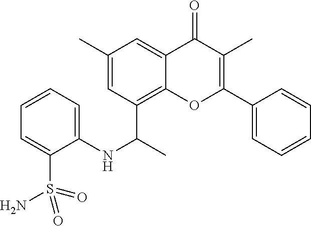 [SMILES: Cc1cc(C(C)Nc2ccccc2S(N)(=O)=O)c2oc(-c3ccccc3)c(C)c(=O)c2c1] | 449 [M + H]⁺ |
| 34A ^{c} | 2-[1-(3,6-Dimethyl-4- oxo-2-phenyl-chromen- 8- yl)ethylamino]benzene sulfonamide, Isomer 2 | 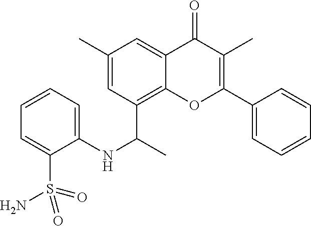 [SMILES: Cc1cc(C(C)Nc2ccccc2S(N)(=O)=O)c2oc(-c3ccccc3)c(C)c(=O)c2c1] | 449 [M + H]⁺ |
| 35A | 8-[1-[2-(3- Hydroxyoxetan-3- yl)anilino]ethyl]-3,6- dimethyl-2-phenyl- chromen-4-one | 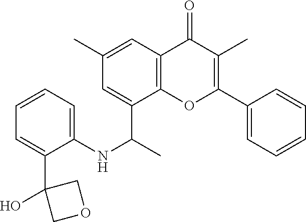 [SMILES: Cc1cc(C(C)Nc2ccccc2C2(O)COC2)c2oc(-c3ccccc3)c(C)c(=O)c2c1] | 442 [M + H]⁺ |
| 36A ^{d} | 8-[1-[2-(3- Hydroxyoxetan-3- yl)anilino]ethyl]-3,6- dimethyl-2-phenyl- chromen-4-one, Isomer 1 | 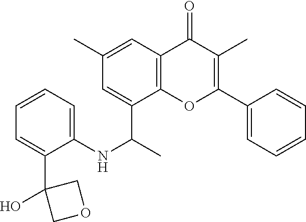 [SMILES: Cc1cc(C(C)Nc2ccccc2C2(O)COC2)c2oc(-c3ccccc3)c(C)c(=O)c2c1] | 442 [M + H]⁺ |
| 37A ^{d} | 8-[1-[2-(3- Hydroxyoxetan-3- yl)anilino]ethyl]-3,6- dimethyl-2-phenyl- chromen-4-one, Isomer 2 | 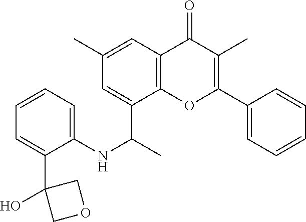 [SMILES: Cc1cc(C(C)Nc2ccccc2C2(O)COC2)c2oc(-c3ccccc3)c(C)c(=O)c2c1] | 442 [M + H]⁺ |

^{a} [Chiralpak AD, 20 × 250 mm; 35% IPA (0.5% DMEA) in CO₂]
^{b} [Chiralpak IH, 20 × 250 mm; 30% MeOH (0.5% DMEA) in CO₂]
^{c} [Chiralpak AS-H, 21 × 150 mm; 20% MeOH in CO₂]
^{d} [Chiralpak IG, 30 × 250 mm; 30% EtOH in CO₂]

The following compound in Table 58 was made in a similar way as described for 3-[[(1R)-1-(3,6-dimethyl-4-oxo-2-phenyl-chromen-8-yl)ethyl]amino]-N′-hydroxy-pyridine-2-carboxamidine.

TABLE 58

| Example | | | MS ES+ |
|---|---|---|---|
| # | Chemical Name | Structure | m/z |
| 38A | 2-[[(1R)-1-(3,6- Dimethyl-4-oxo-2- phenyl-chromen-8- yl)ethyl]amino]-N′- hydroxy-benzamidine | 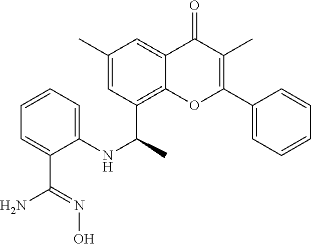 [SMILES: Cc1cc([C@@H](C)Nc2ccccc2/C(N)=N/O)c2oc(-c3ccccc3)c(C)c(=O)c2c1] | 428 [M + H]⁺ |

### Example 39A: 3-[2-[[(1R)-1-(3,6-Dimethyl-4-oxo-2-phenyl-chromen-8-yl)ethyl]amino]phenyl]-4H-1,2,4-oxadiazol-5-one

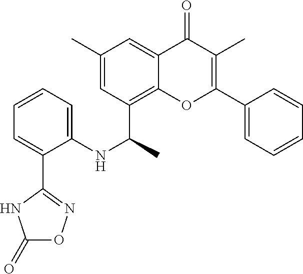 [SMILES: Cc1cc([C@@H](C)Nc2ccccc2-c2noc(=O)[nH]2)c2oc(-c3ccccc3)c(C)c(=O)c2c1]

To a solution of 2-[[(1R)-1-(3,6-dimethyl-4-oxo-2-phenyl-chromen-8-yl)ethyl]amino]-N′-hydroxy-benzamidine (0.100 g, 0.234 mmol) in 1,4-dioxane (2 mL) was added 1,8-diazabicyclo[5.4.0]undec-7-ene (0.071 mL, 0.468 mmol) and di(1H-imidazol-1-yl)methanone (0.057 g, 0.351 mmol). The reaction was sealed and heated in a microwave reactor at 100° C. for 1 h. The mixture was concentrated and purified by reverse phase chromatography eluted with a gradient of 0% to 100% ACN in H₂O+0.1% formic acid. Fractions containing the desired product were combined, washed with saturated aqueous sodium chloride, and extracted with IPA:CHCl₃ (1:3). The combined extracts were dried over MgSO₄, filtered, and concentrated. The material was recrystallized from a mixture of DCM and hexanes to give the title compound (0.052 g, 49%) as a white solid. MS ES+ m/z 454 [M+H]⁺.

The following compounds in Table 59 were made in a similar way as described for 3-[2-[[(1R)-1-(3,6-dimethyl-4-oxo-2-phenyl-chromen-8-yl)ethyl]amino]phenyl]-4H-1,2,4-oxadiazol-5-one. Various methods were used to purify the compounds, which would be apparent to one skilled in the art.

TABLE 59

| Example | | | MS ES+ |
|---|---|---|---|
| # | Chemical Name | Structure | m/z |
| 40A | 3-[3-[[(1R)-1-(3,6-Dimethyl- 4-oxo-2-phenyl-chromen-8- yl)ethyl]amino]-2-pyridyl]- 4H-1,2,4-oxadiazol-5-one | 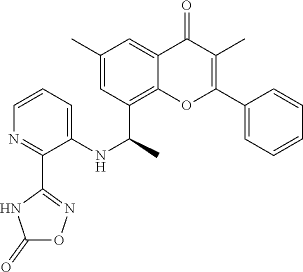 [SMILES: Cc1cc([C@@H](C)Nc2cccnc2-c2noc(=O)[nH]2)c2oc(-c3ccccc3)c(C)c(=O)c2c1] | 455 [M + H]⁺ |
| 41A | 3-[3-[[(1R)-1-(3,6-Dimethyl- 4-oxo-2-phenyl-chromen-8- yl)ethyl]amino]-6-methyl-2- pyridyl]-4H-1,2,4-oxadiazol- 5-one | 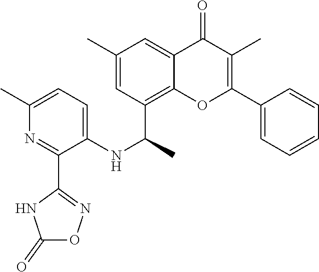 [SMILES: Cc1cc([C@@H](C)Nc2ccc(C)nc2-c2noc(=O)[nH]2)c2oc(-c3ccccc3)c(C)c(=O)c2c1] | 469 [M+H] |
| 42A | 3-[3-[[(1R)-1-[3,6-Dimethyl- 4-oxo-2-(3-pyridyl)chromen- 8-yl]ethyl]amino]-2-pyridyl]- 4H-1,2,4-oxadiazol-5-one | 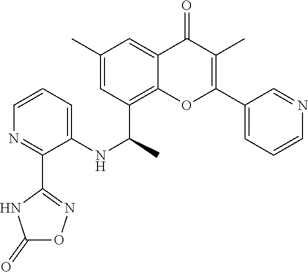 [SMILES: Cc1cc([C@@H](C)Nc2cccnc2-c2noc(=O)[nH]2)c2oc(-c3cccnc3)c(C)c(=O)c2c1] | 456 [M + H]⁺ |
| 119A | 3-[6-Chloro-3-[(1R)-1-[3,6- dimethyl-4-oxo-2-(2- pyridyl)chromen-8- yl]ethyl]amino]-2-pyridyl]- 4H-1,2,4-oxadiazol-5-one | 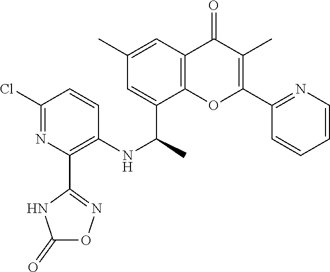 [SMILES: Cc1cc([C@@H](C)Nc2ccc(Cl)nc2-c2noc(=O)[nH]2)c2oc(-c3ccccn3)c(C)c(=O)c2c1] | 490 [M + H]⁺ |
| 120A | 3-[6-Chloro-3-[(1R)-1-(3,6- dimethyl-4-oxo-2-phenyl- chromen-8-yl)ethyl]amino]-2- pyridyl]-4H-1,2,4-oxadiazol- 5-one | 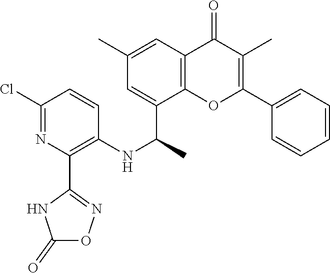 [SMILES: Cc1cc([C@@H](C)Nc2ccc(Cl)nc2-c2noc(=O)[nH]2)c2oc(-c3ccccc3)c(C)c(=O)c2c1] | 489 [M + H]⁺ |
| 121A | 3-[6-[[(1R)-1-(3,6-Dimethyl- 4-oxo-2-phenyl-chromen-8- yl)ethyl]amino]-2,3-difluoro- phenyl]-4H-1,2,4-oxadiazol- 5-one | 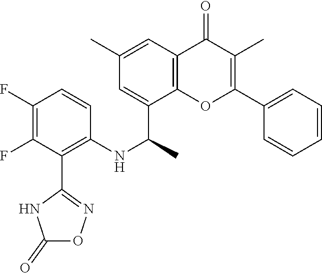 [SMILES: Cc1cc([C@@H](C)Nc2ccc(F)c(F)c2-c2noc(=O)[nH]2)c2oc(-c3ccccc3)c(C)c(=O)c2c1] | 490 [M + H]⁺ |
| 122A | 3-[6-Chloro-3-[[(1R)-1-[2- (3,4-difluorophenyl)-3,6- dimethyl-4-oxo-chromen-8- yl]ethyl]amino]-2-pyridyl]- 4H-1,2,4-oxadiazol-5-one | 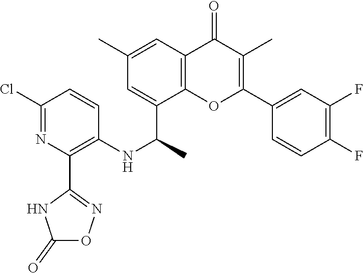 [SMILES: Cc1cc([C@@H](C)Nc2ccc(Cl)nc2-c2noc(=O)[nH]2)c2oc(-c3ccc(F)c(F)c3)c(C)c(=O)c2c1] | 525 [M + H]⁺ |
| 123A | 3-[6-Chloro-3-[[(1R)-1-[2-(5- fluoro-3-pyridyl)-3,6- dimethyl-4-oxo-chromen-8- ylethyl]amino]-2-pyridyl]- 4H-1,2,4-oxadiazol-5-one | 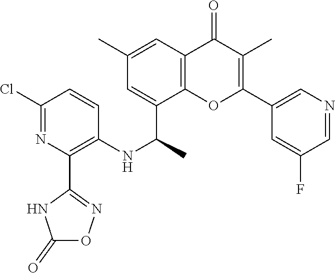 [SMILES: Cc1cc([C@@H](C)Nc2ccc(Cl)nc2-c2noc(=O)[nH]2)c2oc(-c3cncc(F)c3)c(C)c(=O)c2c1] | 508 [M + H]⁺ |
| 124A | 3-[2-Fluoro-6-[(1R)-1-[2-(3- fluorophenyl)-3,6-dimethyl-4- oxo-chromen-8- yl]ethyl]amino]phenyl]-4H- 1,2,4-oxadiazol-5-one | 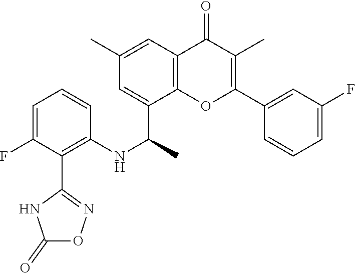 [SMILES: Cc1cc([C@@H](C)Nc2cccc(F)c2-c2noc(=O)[nH]2)c2oc(-c3cccc(F)c3)c(C)c(=O)c2c1] | 490 [M + H]⁺ |
| 125A | 3-[2,3-Difluoro-6-[(1R)-1-[2- (5-fluoro-3-pyridyl)-3,6- dimethyl-4-oxo-chromen-8- yl]ethyl]amino]phenyl]-4H- 1,2,4-oxadiazol-5-one | 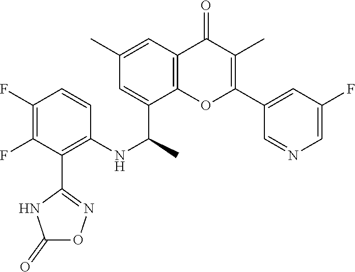 [SMILES: Cc1cc([C@@H](C)Nc2ccc(F)c(F)c2-c2noc(=O)[nH]2)c2oc(-c3cncc(F)c3)c(C)c(=O)c2c1] | 509 [M + H]⁺ |
| 126A | 3-[2-[[(1R)-1-(3,6-Dimethyl- 4-oxo-2-phenyl-chromen-8- yl)ethyl]amino]-6-fluoro- phenyl]-4H-1,2,4-oxadiazol- 5-one | 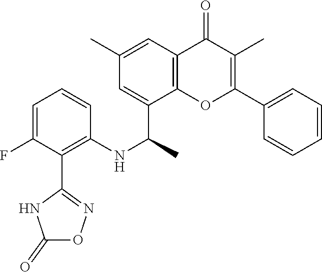 [SMILES: Cc1cc([C@@H](C)Nc2cccc(F)c2-c2noc(=O)[nH]2)c2oc(-c3ccccc3)c(C)c(=O)c2c1] | 472 [M + H]⁺ |
| 127A | 3-[2,3-Difluoro-6-[(1R)-1-[2- (2-fluorophenyl)-3,6- dimethyl-4-oxo-chromen-8- yl]ethyl]amino]phenyl]-4H- 1,2,4-oxadiazol-5-one | 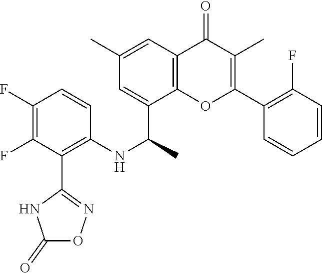 [SMILES: Cc1cc([C@@H](C)Nc2ccc(F)c(F)c2-c2noc(=O)[nH]2)c2oc(-c3ccccc3F)c(C)c(=O)c2c1] | 508 [M + H]⁺ |
| 128A | 3-[6-[[(1R)-1-[3,6-Dimethyl- 4-oxo-2-(3-pyridyl)chromen- 8-yl]ethyl]amino]-2,3- difluoro-phenyl]-4H-1,2,4- oxadiazol-5-one | 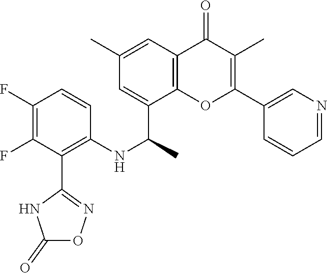 [SMILES: Cc1cc([C@@H](C)Nc2ccc(F)c(F)c2-c2noc(=O)[nH]2)c2oc(-c3cccnc3)c(C)c(=O)c2c1] | 491 [M + H]⁺ |
| 129A | 3-[2-Fluoro-6-[(1R)-1-[2-(2- fluorophenyl)-3,6-dimethyl-4- oxo-chromen-8- yl]ethyl]amino]phenyl]-4H- 1,2,4-oxadiazol-5-one | 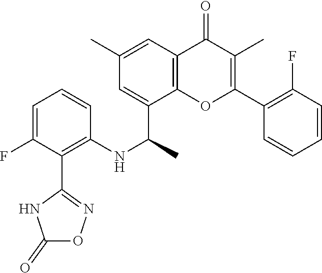 [SMILES: Cc1cc([C@@H](C)Nc2cccc(F)c2-c2noc(=O)[nH]2)c2oc(-c3ccccc3F)c(C)c(=O)c2c1] | 490 [M + H]⁺ |
| 130A | 3-[2-[[(1R)-1-(3,6-Dimethyl- 4-oxo-2-phenyl-chromen-8- yl)ethyl]amino]-5-fluoro- phenyl]-4H-1,2,4-oxadiazol- 5-one | 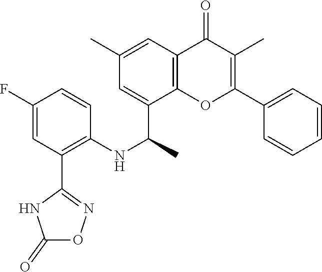 [SMILES: Cc1cc([C@@H](C)Nc2ccc(F)cc2-c2noc(=O)[nH]2)c2oc(-c3ccccc3)c(C)c(=O)c2c1] | 472 [M + H]⁺ |
| 131A | 3-[3-Chloro-6-[[(1R)-1-(3,6- dimethyl-4-oxo-2-phenyl- chromen-8-yl)ethyl]amino]-2- fluoro-phenyl]-4H-1,2,4- oxadiazol-5-one | 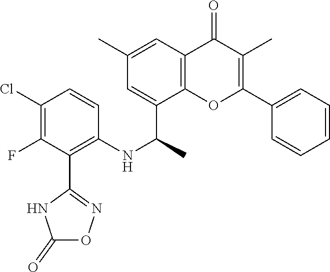 [SMILES: Cc1cc([C@@H](C)Nc2ccc(Cl)c(F)c2-c2noc(=O)[nH]2)c2oc(-c3ccccc3)c(C)c(=O)c2c1] | 506 [M + H]⁺ |
| 132A | 3-[3-[[(1R)-1-[2-(5-Fluoro-3- pyridyl)-3,6-dimethyl-4-oxo- chromen-8-yl]ethyl]amino]-2- pyridyl]-4H-1,2,4-oxadiazol- 5-one | 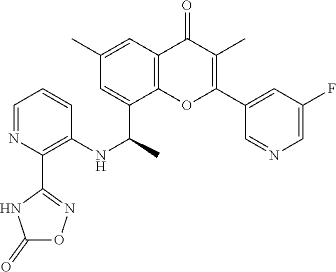 [SMILES: Cc1cc([C@@H](C)Nc2cccnc2-c2noc(=O)[nH]2)c2oc(-c3cncc(F)c3)c(C)c(=O)c2c1] | 474 [M + H]⁺ |
| 133A | 3-[5-Chloro-2-[(1R)-1-[3,6- dimethyl-4-oxo-2-(3- pyridyl)chromen-8- yl]ethyl]amino]phenyl]-4H- 1,2,4-oxadiazol-5-one | 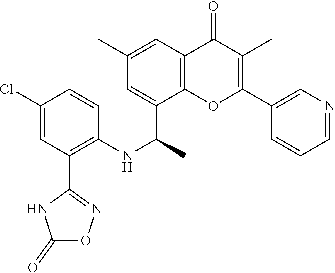 [SMILES: Cc1cc([C@@H](C)Nc2ccc(Cl)cc2-c2noc(=O)[nH]2)c2oc(-c3cccnc3)c(C)c(=O)c2c1] | 489 [M + H]⁺ |
| 134A | 3-[3-[[(1R)-1-[2-(3,4- Difluorophenyl)-3,6- dimethyl-4-oxo-chromen-8- yl]ethyl]amino]-6-methyl-2- pyridyl]-4H-1,2,4-oxadiazol- 5-one | 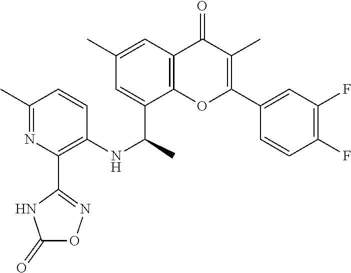 [SMILES: Cc1cc([C@@H](C)Nc2ccc(C)nc2-c2noc(=O)[nH]2)c2oc(-c3ccc(F)c(F)c3)c(C)c(=O)c2c1] | 505 [M + H]⁺ |
| 135A | 3-[3-[[(1R)-1-[3,6-Dimethyl- 2-(1-methylpyrazol-4-yl)-4- oxo-chromen-8- yl]ethyl]amino]-2-pyridyl]- 4H-1,2,4-oxadiazol-5-one | 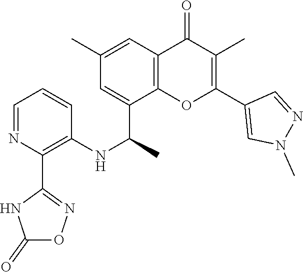 [SMILES: Cc1cc([C@@H](C)Nc2cccnc2-c2noc(=O)[nH]2)c2oc(-c3cnn(C)c3)c(C)c(=O)c2c1] | 459 [M + H]⁺ |
| 136A | 3-[3-[[(1R)-1-[2-(2-Fluoro-3- pyridyl)-3,6-dimethyl-4-oxo- chromen-8-yl]ethyl]amino]-2- pyridyl]-4H-1,2,4-oxadiazol- 5-one | 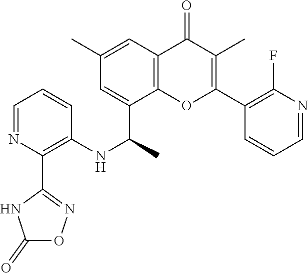 [SMILES: Cc1cc([C@@H](C)Nc2cccnc2-c2noc(=O)[nH]2)c2oc(-c3cccnc3F)c(C)c(=O)c2c1] | 474 [M + H]⁺ |
| 137A | 3-[3-[(1R)-1-[2-(2- Fluorophenyl)-3,6-dimethyl- 4-oxo-chromen-8- yl]ethyl]amino]-2-pyridyl]- 4H-1,2,4-oxadiazol-5-one | 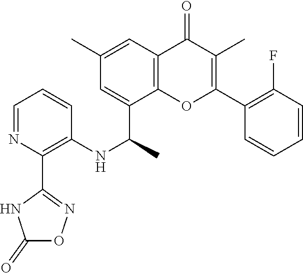 [SMILES: Cc1cc([C@@H](C)Nc2cccnc2-c2noc(=O)[nH]2)c2oc(-c3ccccc3F)c(C)c(=O)c2c1] | 473 [M + H]⁺ |
| 138A | 3-[2-[[(1R)-1-[2-(2,3- Difluorophenyl)-3,6- dimethyl-4-oxo-chromen-8- yl]ethyl]amino]-6-fluoro- phenyl]-4H-1,2,4-oxadiazol- 5-one | 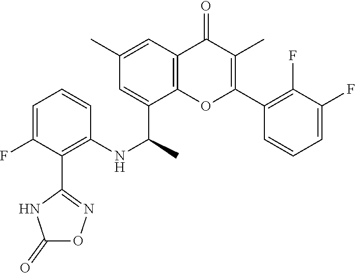 [SMILES: Cc1cc([C@@H](C)Nc2cccc(F)c2-c2noc(=O)[nH]2)c2oc(-c3cccc(F)c3F)c(C)c(=O)c2c1] | 508 [M + H]⁺ |
| 139A | 3-[3-[[(1R)-1-[3,6-Dimethyl- 2-(2-methylindazol-5-yl)-4- oxo-chromen-8- yl]ethyl]amino]-2-pyridyl]- 4H-1,2,4-oxadiazol-5-one | 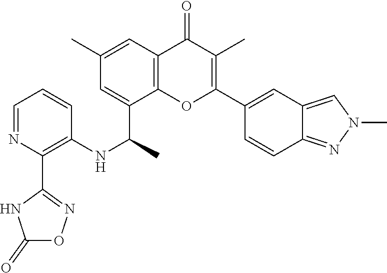 [SMILES: Cc1cc([C@@H](C)Nc2cccnc2-c2noc(=O)[nH]2)c2oc(-c3ccc4nn(C)cc4c3)c(C)c(=O)c2c1] | 509 [M + H]⁺ |
| 140A | 3-[2-[[(1R)-1-[2-(2,4- Difluorophenyl)-3,6- dimethyl-4-oxo-chromen-8- yl]ethyl]amino]-6-fluoro- phenyl]-4H-1,2,4-oxadiazol- 5-one | 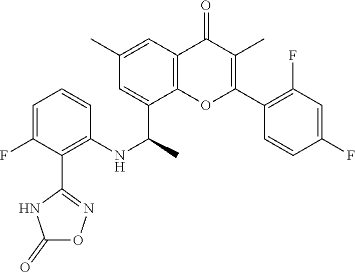 [SMILES: Cc1cc([C@@H](C)Nc2cccc(F)c2-c2noc(=O)[nH]2)c2oc(-c3ccc(F)cc3F)c(C)c(=O)c2c1] | 508 [M + H]⁺ |
| 141A | 3-[3-[[(1R)-1-[2-[6- (Difluoromethyl)-2-pyridyl]- 3,6-dimethyl-4-oxo-chromen- 8-yl]ethyl]amino]-2-pyridyl]- 4H-1,2,4-oxadiazol-5-one | 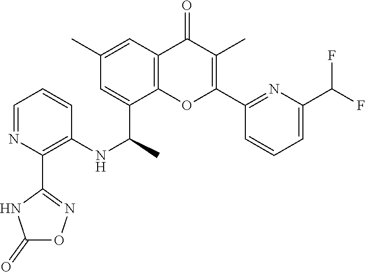 [SMILES: Cc1cc([C@@H](C)Nc2cccnc2-c2noc(=O)[nH]2)c2oc(-c3cccc(C(F)F)n3)c(C)c(=O)c2c1] | 506 [M + H]⁺ |
| 142A | 3-[2-Chloro-6-[(1R)-1-(3,6- dimethyl-4-oxo-2-phenyl- chromen-8- yl)ethyl]amino]phenyl]-4H- 1,2,4-oxadiazol-5-one | 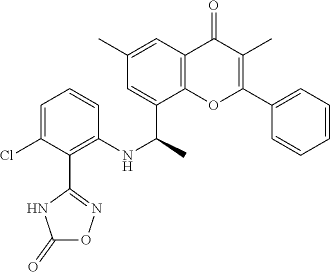 [SMILES: Cc1cc([C@@H](C)Nc2cccc(Cl)c2-c2noc(=O)[nH]2)c2oc(-c3ccccc3)c(C)c(=O)c2c1] | 488 [M + H]⁺ |
| 143A | 3-[3-[(1R)-1-[2-[6- (Difluoromethyl)-2-pyridyl]- 3,6-dimethyl-4-oxo-chromen- 8-yl]ethyl]amino]-6-methyl-2- pyridyl]-4H-1,2,4-oxadiazol- 5-one | 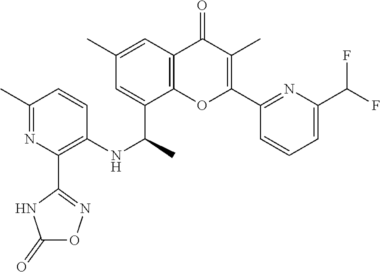 [SMILES: Cc1cc([C@@H](C)Nc2ccc(C)nc2-c2noc(=O)[nH]2)c2oc(-c3cccc(C(F)F)n3)c(C)c(=O)c2c1] | 520 [M + H]⁺ |
| 144A | 3-[6-Chloro-3-[[(1R)-1-[3,6- dimethyl-2-(1-methylpyrazol- 4-yl)-4-oxo-chromen-8- yl]ethyl]amino]-2-pyridyl]- 4H-1,2,4-oxadiazol-5-one | 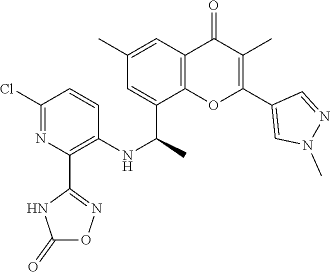 [SMILES: Cc1cc([C@@H](C)Nc2ccc(Cl)nc2-c2noc(=O)[nH]2)c2oc(-c3cnn(C)c3)c(C)c(=O)c2c1] | 493 [M + H]⁺ |
| 145A | 3-[6-Chloro-3-[[(1R)-1-[2-(6- fluoro-2-pyridyl)-3,6- dimethyl-4-oxo-chromen-8- yl]ethyl]amino]-2-pyridyl]- 4H-1,2,4-oxadiazol-5-one | 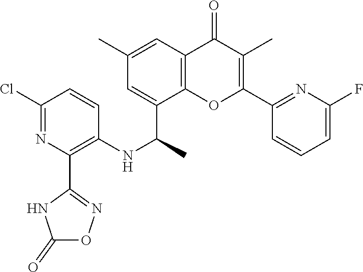 [SMILES: Cc1cc([C@@H](C)Nc2ccc(Cl)nc2-c2noc(=O)[nH]2)c2oc(-c3cccc(F)n3)c(C)c(=O)c2c1] | 508 [M + H]⁺ |
| 146A | 3-[6-Chloro-3-[(1R)-1-[3,6- dimethyl-4-oxo-2-(3- pyridyl)chromen-8- yl]ethyl]amino]-2-pyridyl]- 4H-1,2,4-oxadiazol-5-one | 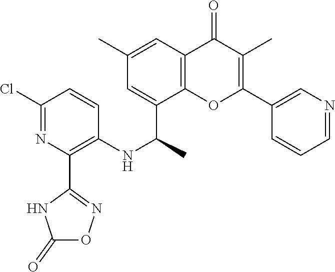 [SMILES: Cc1cc([C@@H](C)Nc2ccc(Cl)nc2-c2noc(=O)[nH]2)c2oc(-c3cccnc3)c(C)c(=O)c2c1] | 490 [M + H]⁺ |
| 147A | 3-[6-Chloro-3-[[(1R)-1-[2-(2- fluoro-3-pyridyl)-3,6- dimethyl-4-oxo-chromen-8- yl]ethyl]amino]-2-pyridyl]- 4H-1,2,4-oxadiazol-5-one | 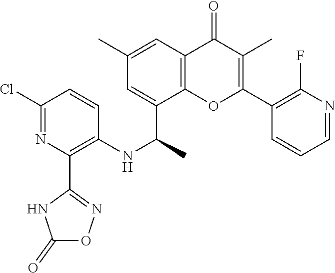 [SMILES: Cc1cc([C@@H](C)Nc2ccc(Cl)nc2-c2noc(=O)[nH]2)c2oc(-c3cccnc3F)c(C)c(=O)c2c1] | 508 [M + H]⁺ |
| 148A | 3-[6-Chloro-3-[(1R)-1-[2-(3- fluoro-2-pyridyl)-3,6- dimethyl-4-oxo-chromen-8- yl]ethyl]amino]-2-pyridyl]- 4H-1,2,4-oxadiazol-5-one | 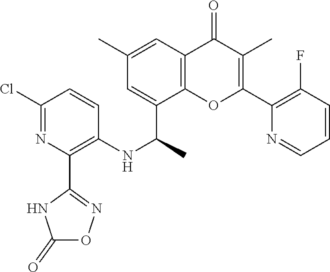 [SMILES: Cc1cc([C@@H](C)Nc2ccc(Cl)nc2-c2noc(=O)[nH]2)c2oc(-c3ncccc3F)c(C)c(=O)c2c1] | 508 [M + H]⁺ |
| 149A | 3-[6-Chloro-3-[(1R)-1-[2- (2,5-difluoro-3-pyridyl)-3,6- dimethyl-4-oxo-chromen-8- yl]ethyl]amino]-2-pyridyl]- 4H-1,2,4-oxadiazol-5-one | 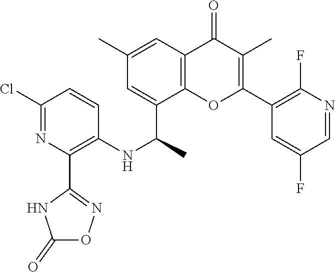 [SMILES: Cc1cc([C@@H](C)Nc2ccc(Cl)nc2-c2noc(=O)[nH]2)c2oc(-c3cc(F)cnc3F)c(C)c(=O)c2c1] | 526 [M + H]⁺ |
| 150A | 3-[6-Chloro-3-[(1R)-1-[2-(6- methoxy-2-pyridyl)-3,6- dimethyl-4-oxo-chromen-8- yl]ethyl]amino]-2-pyridyl]- | 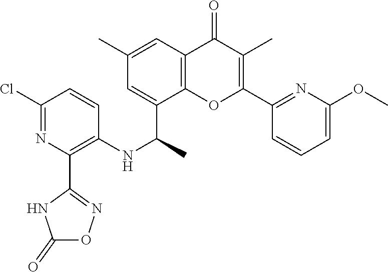 [SMILES: COc1cccc(-c2oc3c([C@@H](C)Nc4ccc(Cl)nc4-c4noc(=O)[nH]4)cc(C)cc3c(=O)c2C)n1] | 520 [M + H]⁺ |
| 151A | 3-[6-Chloro-3-[(1R)-1-[3- methyl-4-oxo-2-(2-pyridyl)-6- (trifluoromethyl)chromen-8- yl]ethyl]amino]-2-pyridyl]- 4H-1,2,4-oxadiazol-5-one | 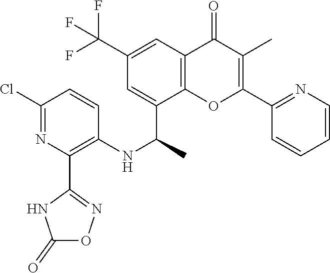 [SMILES: Cc1c(-c2ccccn2)oc2c([C@@H](C)Nc3ccc(Cl)nc3-c3noc(=O)[nH]3)cc(C(F)(F)F)cc2c1=O] | 544 [M + H]⁺ |
| 152A | 3-[6-Chloro-3-[[(1R)-1-[6- methyl-2-(1-methylpyrazol-4- yl)-4-oxo-chromen-8- yl]ethyl]amino]-2-pyridyl]- 4H-1,2,4-oxadiazol-5-one | 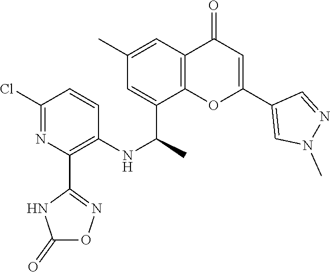 [SMILES: Cc1cc([C@@H](C)Nc2ccc(Cl)nc2-c2noc(=O)[nH]2)c2oc(-c3cnn(C)c3)cc(=O)c2c1] | 479 [M + H]⁺ |
| 153A | 3-[3-[(1R)-1-[2-(5-Fluoro-3- pyridyl)-3-methyl-4-oxo-6- (trifluoromethyl)chromen-8- yl]ethyl]amino]-2-pyridyl]- 4H-1,2,4-oxadiazol-5-one | 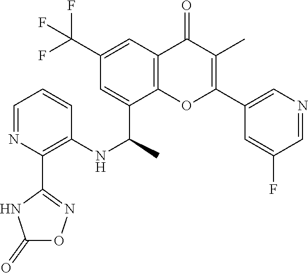 [SMILES: Cc1c(-c2cncc(F)c2)oc2c([C@@H](C)Nc3cccnc3-c3noc(=O)[nH]3)cc(C(F)(F)F)cc2c1=O] | 528 [M + H]⁺ |
| 154A | 3-[3-[[(1R)-1-[3-Methyl-4- oxo-2-(2-pyridyl)-6- (trifluoromethyl)chromen-8- yl]ethyl]amino]-2-pyridyl]- 4H-1,2,4-oxadiazol-5-one | 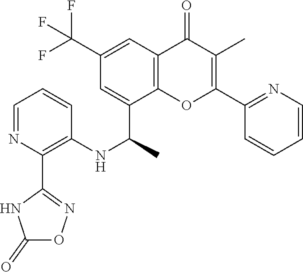 [SMILES: Cc1c(-c2ccccn2)oc2c([C@@H](C)Nc3cccnc3-c3noc(=O)[nH]3)cc(C(F)(F)F)cc2c1=O] | 510 [M + H]⁺ |
| 155A | 3-[6-[[(1R)-1-(3,6-Dimethyl- 4-oxo-2-pyrimidin-5-yl- chromen-8-yl)ethyl]amino]- 2,3-difluoro-phenyl]-4H- 1,2,4-oxadiazol-5-one | 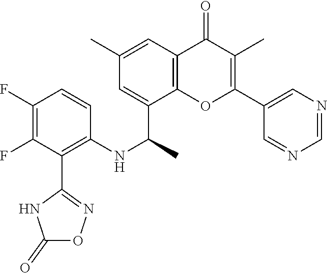 [SMILES: Cc1cc([C@@H](C)Nc2ccc(F)c(F)c2-c2noc(=O)[nH]2)c2oc(-c3cncnc3)c(C)c(=O)c2c1] | 490 [M − H]⁻ |
| 4C | 3-[6-Chloro-3-[[(1R)-1-[3,6- dimethyl-4-oxo-2-(1H- pyrazol-4-yl)chromen-8- yl]ethyl]amino]-2-pyridyl]- 4H-1,2,4-oxadiazol-5-one | 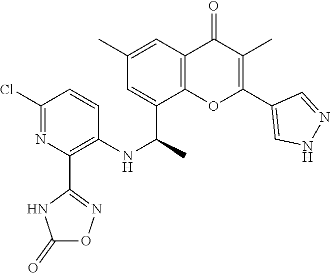 [SMILES: Cc1cc([C@@H](C)Nc2ccc(Cl)nc2-c2noc(=O)[nH]2)c2oc(-c3cn[nH]c3)c(C)c(=O)c2c1] | 479 [M + H]⁺ |
| 5C | 3-[6-Chloro-3-[(1R)-1-[2- (2,5-dimethylpyrazol-3-yl)- 3,6-dimethyl-4-oxo-chromen- 8-yl]ethyl]amino]-2-pyridyl]- 4H-1,2,4-oxadiazol-5-one | 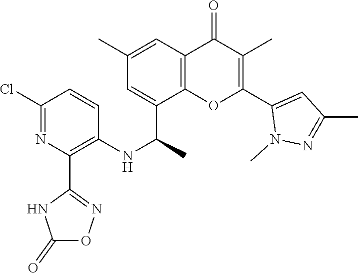 [SMILES: Cc1cc([C@@H](C)Nc2ccc(Cl)nc2-c2noc(=O)[nH]2)c2oc(-c3cc(C)nn3C)c(C)c(=O)c2c1] | 507 [M + H]⁺ |
| 6C | 3-[6-Chloro-3-[(1R)-1-[3,6- dimethyl-4-oxo-2-[1- (trifluoromethyl)pyrazol-4- yl]chromen-8- yl]ethyl]amino]-2-pyridyl]- 4H-1,2,4-oxadiazol-5-one | 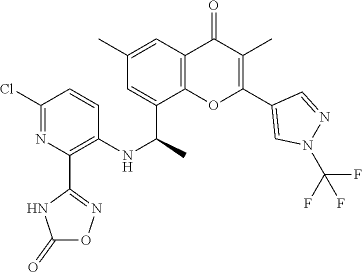 [SMILES: Cc1cc([C@@H](C)Nc2ccc(Cl)nc2-c2noc(=O)[nH]2)c2oc(-c3cnn(C(F)(F)F)c3)c(C)c(=O)c2c1] | 547 [M + H]⁺ |
| 7C | 3-[6-Chloro-3-[[(1R)-1-(3,6- dimethyl-4-oxo-2-pyrazin-2- yl-chromen-8- yl)ethyl]amino]-2-pyridyl]- 4H-1,2,4-oxadiazol-5-one | 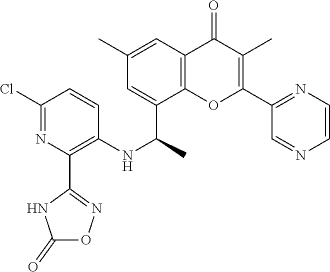 [SMILES: Cc1cc([C@@H](C)Nc2ccc(Cl)nc2-c2noc(=O)[nH]2)c2oc(-c3cnccn3)c(C)c(=O)c2c1] | 491 [M + H]⁺ |
| 8C | 3-[6-Chloro-3-[[(1R)-1-[3,6- dimethyl-2-(2-methyltriazol- 4-yl)-4-oxo-chromen-8- yl]ethyl]amino]-2-pyridyl]- 4H-1,2,4-oxadiazol-5-one | 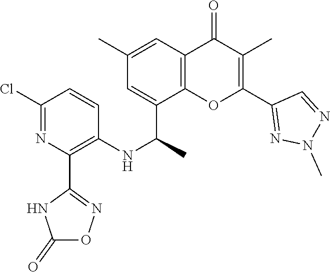 [SMILES: Cc1cc([C@@H](C)Nc2ccc(Cl)nc2-c2noc(=O)[nH]2)c2oc(-c3cnn(C)n3)c(C)c(=O)c2c1] | 494 [M + H]⁺ |
| 9C | 3-[6-Chloro-3-[(1R)-1-[2-(3- fluoro-1-methyl-pyrazol-4- yl)-3,6-dimethyl-4-oxo- chromen-8-yl]ethyl]amino]-2- pyridyl]-4H-1,2,4-oxadiazol- 5-one | 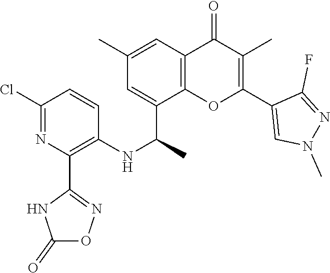 [SMILES: Cc1cc([C@@H](C)Nc2ccc(Cl)nc2-c2noc(=O)[nH]2)c2oc(-c3cn(C)nc3F)c(C)c(=O)c2c1] | 511 [M + H]⁺ |
| 10C | 3-[6-Chloro-3-[[(1R)-1-(6- methyl-3-oxazol-4-yl-4-oxo- 2-phenyl-chromen-8- yl)ethyl]amino]-2-pyridyl]- 4H-1,2,4-oxadiazol-5-one | 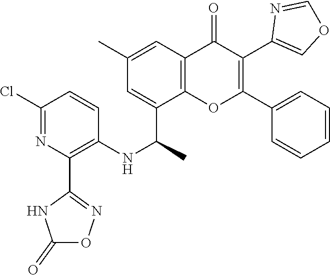 [SMILES: Cc1cc([C@@H](C)Nc2ccc(Cl)nc2-c2noc(=O)[nH]2)c2oc(-c3ccccc3)c(-c3cocn3)c(=O)c2c1] | 542 [M + H]⁺ |
| 11C | 3-[6-Chloro-3-[[(1R)-1-[2-[1- (2-methoxyethyl)pyrazol-4- yl]-3,6-dimethyl-4-oxo- chromen-8-yl]ethyl]amino]-2- pyridyl]-4H-1,2,4-oxadiazol- 5-one | 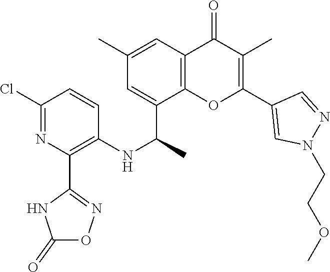 [SMILES: COCCn1cc(-c2oc3c([C@@H](C)Nc4ccc(Cl)nc4-c4noc(=O)[nH]4)cc(C)cc3c(=O)c2C)cn1] | 537 [M + H]⁺ |
| 12C | 3-[6-Chloro-3-[(1R)-1-[2-[1- (2-hydroxy-2-methyl- propyl)pyrazol-4-yl]-3,6- dimethyl-4-oxo-chromen-8- yl]ethyl]amino]-2-pyridyl]- 4H-1,2,4-oxadiazol-5-one | 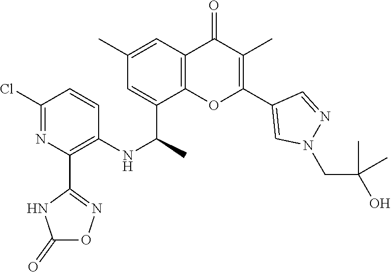 [SMILES: Cc1cc([C@@H](C)Nc2ccc(Cl)nc2-c2noc(=O)[nH]2)c2oc(-c3cnn(CC(C)(C)O)c3)c(C)c(=O)c2c1] | 551 [M + H]⁺ |
| 13C | 3-[6-Chloro-3-[[(1R)-1-[3,6- dimethyl-4-oxo-2-(1- tetrahydropyran-4-ylpyrazol- 4-yl)chromen-8- ylethyl]amino]-2-pyridyl]- 4H-1,2,4-oxadiazol-5-one | 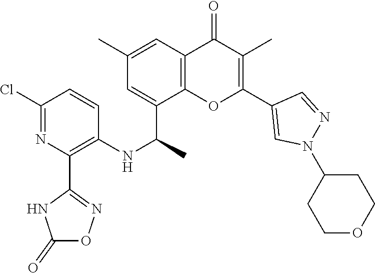 [SMILES: Cc1cc([C@@H](C)Nc2ccc(Cl)nc2-c2noc(=O)[nH]2)c2oc(-c3cnn(C4CCOCC4)c3)c(C)c(=O)c2c1] | 563 [M + H]⁺ |
| 14C | 3-[6-Chloro-3-[(1R)-1-[2- (1,5-dimethylpyrazol-4-yl)- 3,6-dimethyl-4-oxo-chromen- 8-yl]ethyl]amino]-2-pyridyl]- 4H-1,2,4-oxadiazol-5-one | 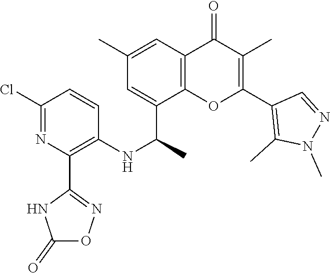 [SMILES: Cc1cc([C@@H](C)Nc2ccc(Cl)nc2-c2noc(=O)[nH]2)c2oc(-c3cnn(C)c3C)c(C)c(=O)c2c1] | 507 [M + H]⁺ |
| 15C | 2-[4-[8-[(1R)-1-[6-Chloro-2- (5-oxo-4H-1,2,4-oxadiazol-3- yl)-3-pyridyl]amino]ethyl]- 3,6-dimethyl-4-oxo-chromen- 2-yl]pyrazol-1-yl]-2-methyl- propanenitrile | 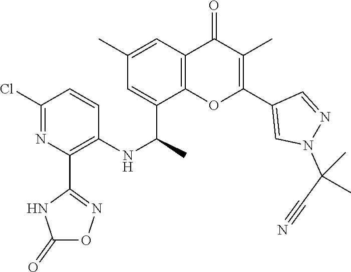 [SMILES: Cc1cc([C@@H](C)Nc2ccc(Cl)nc2-c2noc(=O)[nH]2)c2oc(-c3cnn(C(C)(C)C#N)c3)c(C)c(=O)c2c1] | 546 [M + H]⁺ |
| 16C | 3-[6-Chloro-3-[[(1R)-1-[6- methyl-2-(1-methylpyrazol-4- yl)-3-oxazol-4-yl-4-oxo- chromen-8-yl]ethyl]amino]-2- pyridyl]-4H-1,2,4-oxadiazol- 5-one | 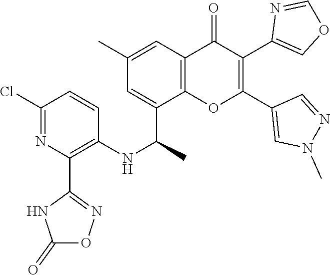 [SMILES: Cc1cc([C@@H](C)Nc2ccc(Cl)nc2-c2noc(=O)[nH]2)c2oc(-c3cnn(C)c3)c(-c3cocn3)c(=O)c2c1] | 546 [M + H]⁺ |
| 17C | 3-[6-Chloro-3-[[(1R)-1-[3,6- dimethyl-2-(1- methylpyrazolo[4,3- b]pyridin-5-yl)-4-oxo- chromen-8-yl]ethyl]amino]-2- pyridyl]-4H-1,2,4-oxadiazol- 5-one | 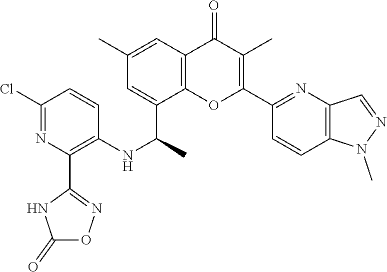 [SMILES: Cc1cc([C@@H](C)Nc2ccc(Cl)nc2-c2noc(=O)[nH]2)c2oc(-c3ccc4c(cnn4C)n3)c(C)c(=O)c2c1] | 544 [M + H]⁺ |
| 18C | 3-[6-Chloro-3-[[(1R)-1-[2- (3,6-difluoro-2-pyridyl)-3,6- dimethyl-4-oxo-chromen-8- yl]ethyl]amino]-2-pyridyl]- 4H-1,2,4-oxadiazol-5-one | 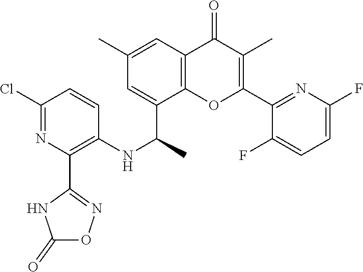 [SMILES: Cc1cc([C@@H](C)Nc2ccc(Cl)nc2-c2noc(=O)[nH]2)c2oc(-c3nc(F)ccc3F)c(C)c(=O)c2c1] | 526 [M + H]⁺ |
| 19C | 3-[6-Chloro-3-[[(1R)-1-[2- (2,4-difluoro-3-pyridyl)-3,6- dimethyl-4-oxo-chromen-8- yl]ethyl]amino]-2-pyridyl]- 4H-1,2,4-oxadiazol-5-one | 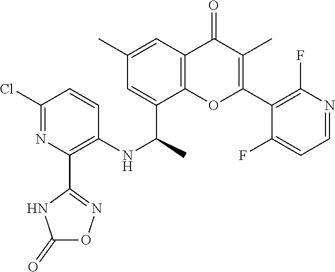 [SMILES: Cc1cc([C@@H](C)Nc2ccc(Cl)nc2-c2noc(=O)[nH]2)c2oc(-c3c(F)ccnc3F)c(C)c(=O)c2c1] | 526 [M + H]⁺ |
| 20C | 3-[6-Chloro-3-[[(1R)-1-[3,6- dimethyl-2-(2-methylpyrazol- 3-yl)-4-oxo-chromen-8- yl]ethyl]amino]-2-pyridyl]- 4H-1,2,4-oxadiazol-5-one | 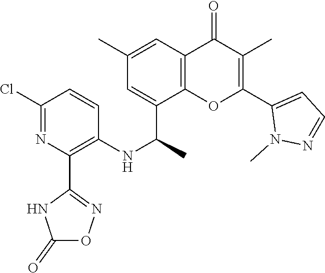 [SMILES: Cc1cc([C@@H](C)Nc2ccc(Cl)nc2-c2noc(=O)[nH]2)c2oc(-c3ccnn3C)c(C)c(=O)c2c1] | 493 [M + H]⁺ |
| 21C | 3-[6-Chloro-3-[[(1R)-1-[2-[1- (difluoromethyl)pyrazol-4- yl]-3,6-dimethyl-4-oxo- chromen-8-yl]ethyl]amino]-2- pyridyl]-4H-1,2,4-oxadiazol- 5-one | 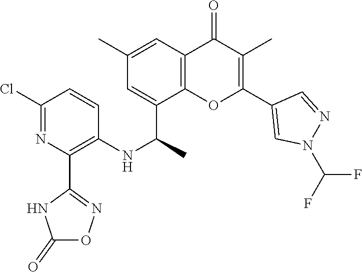 [SMILES: Cc1cc([C@@H](C)Nc2ccc(Cl)nc2-c2noc(=O)[nH]2)c2oc(-c3cnn(C(F)F)c3)c(C)c(=O)c2c1] | 529 [M + H]⁺ |
| 22C | 3-[6-Chloro-3-[[(1R)-1-[2-(1- isopropylpyrazol-4-yl)-3,6- dimethyl-4-oxo-chromen-8- yl]ethyl]amino]-2-pyridyl]- 4H-1,2,4-oxadiazol-5-one | 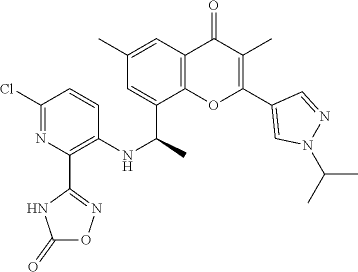 [SMILES: Cc1cc([C@@H](C)Nc2ccc(Cl)nc2-c2noc(=O)[nH]2)c2oc(-c3cnn(C(C)C)c3)c(C)c(=O)c2c1] | 521 [M + H]⁺ |
| 23C | 3-[6-Chloro-3-[(1R)-1-[2-(1- cyclopropylpyrazol-4-yl)-3,6- dimethyl-4-oxo-chromen-8- yl]ethyl]amino]-2-pyridyl]- 4H-1,2,4-oxadiazol-5-one | 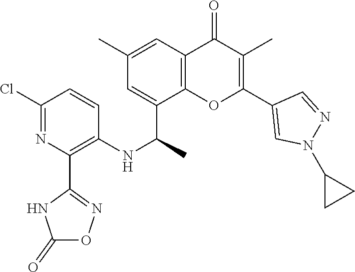 [SMILES: Cc1cc([C@@H](C)Nc2ccc(Cl)nc2-c2noc(=O)[nH]2)c2oc(-c3cnn(C4CC4)c3)c(C)c(=O)c2c1] | 519 [M + H]⁺ |
| 24C | 3-[6-Chloro-3-[(1R)-1-(3,6- dimethyl-4-oxo-2-pyrimidin- 5-yl-chromen-8- yl)ethyl]amino]-2-pyridyl]- 4H-1,2,4-oxadiazol-5-one | 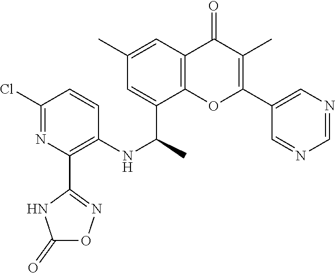 [SMILES: Cc1cc([C@@H](C)Nc2ccc(Cl)nc2-c2noc(=O)[nH]2)c2oc(-c3cncnc3)c(C)c(=O)c2c1] | 491 [M + H]⁺ |
| 25C | 3-[6-Chloro-3-[[(1R)-1-[2-(4- fluoro-3-pyridyl)-3,6- dimethyl-4-oxo-chromen-8- yl]ethyl]amino]-2-pyridyl]- 4H-1,2,4-oxadiazol-5-one | 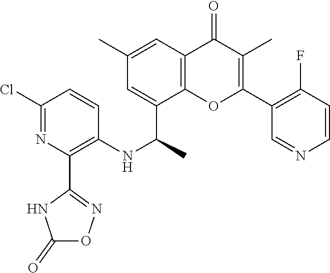 [SMILES: Cc1cc([C@@H](C)Nc2ccc(Cl)nc2-c2noc(=O)[nH]2)c2oc(-c3cnccc3F)c(C)c(=O)c2c1] | 508 [M + H]⁺ |
| 26C | 3-[6-Chloro-3-[[(1R)-1-[2-(5- fluoro-2-pyridyl)-3,6- dimethyl-4-oxo-chromen-8- yl]ethyl]amino]-2-pyridyl]- 4H-1,2,4-oxadiazol-5-one | 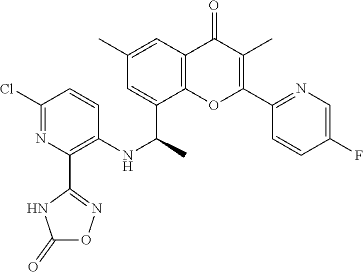 [SMILES: Cc1cc([C@@H](C)Nc2ccc(Cl)nc2-c2noc(=O)[nH]2)c2oc(-c3ccc(F)cn3)c(C)c(=O)c2c1] | 508 [M + H]⁺ |
| 27C | 3-[6-Chloro-3-[[(1R)-1-[3,6- dimethyl-4-oxo-2-[1- (trideuteriomethyl)pyrazol-4- yl]chromen-8- yl]ethyl]amino]-2-pyridyl]- 4H-1,2,4-oxadiazol-5-one | 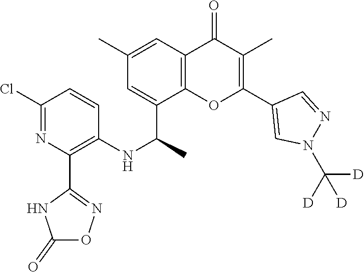 [SMILES: [2H]C([2H])([2H])n1cc(-c2oc3c([C@@H](C)Nc4ccc(Cl)nc4-c4noc(=O)[nH]4)cc(C)cc3c(=O)c2C)cn1] | 496 [M + H]⁺ |
| 28C | 3-[6-Chloro-3-[[(1R)-1-(3,6- dimethyl-4-oxo-2-pyrimidin- 2-yl-chromen-8- yl)ethyl]amino]-2-pyridyl]- 4H-1,2,4-oxadiazol-5-one | 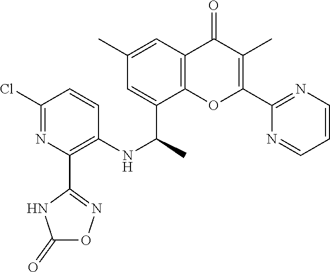 [SMILES: Cc1cc([C@@H](C)Nc2ccc(Cl)nc2-c2noc(=O)[nH]2)c2oc(-c3ncccn3)c(C)c(=O)c2c1] | 491 [M + H]⁺ |
| 29C | 3-[6-Chloro-3-[[(1R)-1-[3,6- dimethyl-2-(1-methylindazol- 3-yl)-4-oxo-chromen-8- yl]ethyl]amino]-2-pyridyl]- 4H-1,2,4-oxadiazol-5-one | 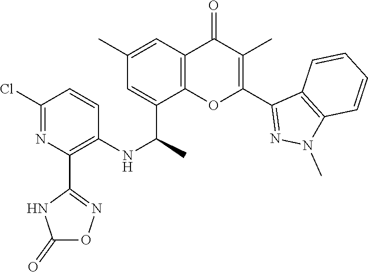 [SMILES: Cc1cc([C@@H](C)Nc2ccc(Cl)nc2-c2noc(=O)[nH]2)c2oc(-c3nn(C)c4ccccc34)c(C)c(=O)c2c1] | 543 [M + H]⁺ |
| 30C | 3-[6-Chloro-3-[[(1R)-1-[2-(6- methoxy-2-pyridyl)-3,6- dimethyl-4-oxo-chromen-8- yl]ethyl]amino]-2-pyridyl]- 4H-1,2,4-oxadiazol-5-one | 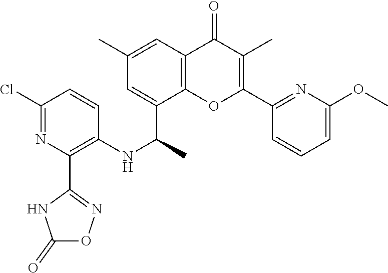 [SMILES: COc1cccc(-c2oc3c([C@@H](C)Nc4ccc(Cl)nc4-c4noc(=O)[nH]4)cc(C)cc3c(=O)c2C)n1] | 520 [M + H]⁺ |
| 31C | 3-[6-Chloro-3-[[(1R)-1-[3,6- dimethyl-2-(2- methylpyrazolo[4,3- b]pyridin-5-yl)-4-oxo- chromen-8-yl]ethyl]amino]-2- pyridyl]-4H-1,2,4-oxadiazol- 5-one | 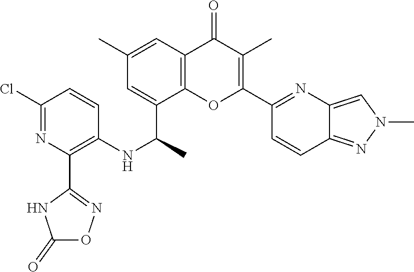 [SMILES: Cc1cc([C@@H](C)Nc2ccc(Cl)nc2-c2noc(=O)[nH]2)c2oc(-c3ccc4nn(C)cc4n3)c(C)c(=O)c2c1] | 544 [M + H]⁺ |
| 32C | 3-[6-Chloro-3-[[(1R)-1- deuterio-1-(3,6-dimethyl-4- oxo-2-phenyl-chromen-8- yl)ethyl]amino]-2-pyridyl]- 4H-1,2,4-oxadiazol-5-one | 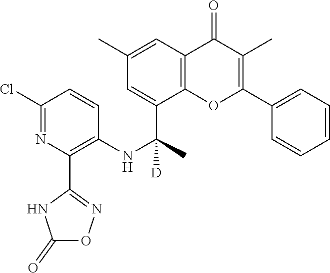 [SMILES: [2H][C@](C)(Nc1ccc(Cl)nc1-c1noc(=O)[nH]1)c1cc(C)cc2c(=O)c(C)c(-c3ccccc3)oc12] | 490 [M + H]⁺ |
| 33C | 3-[3-[(1R)-1-Deuterio-1- (3,6-dimethyl-4-oxo-2- phenyl-chromen-8- yl)ethyl]amino]-2-pyridyl]- 4H-1,2,4-oxadiazol-5-one | 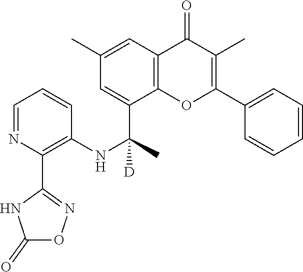 [SMILES: [2H][C@](C)(Nc1cccnc1-c1noc(=O)[nH]1)c1cc(C)cc2c(=O)c(C)c(-c3ccccc3)oc12] | 456 [M + H]⁺ |
| 34C | 3-[6-Chloro-3-[[(1R)-1-[3,6- dimethyl-2-(1-methylindazol- 5-yl)-4-oxo-chromen-8- yl]ethyl]amino]-2-pyridyl]- 4H-1,2,4-oxadiazol-5-one | 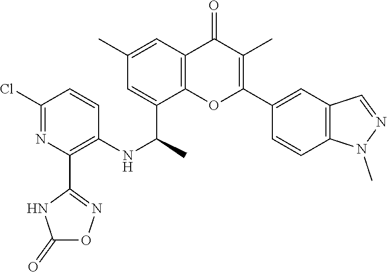 [SMILES: Cc1cc([C@@H](C)Nc2ccc(Cl)nc2-c2noc(=O)[nH]2)c2oc(-c3ccc4c(cnn4C)c3)c(C)c(=O)c2c1] | 543 [M + H]⁺ |
| 35C | 3-[2-Fluoro-6-[(1R)-1-[2-(5- fluoro-3-pyridyl)-3,6- dimethyl-4-oxo-chromen-8- yl]ethyl]amino]phenyl]-4H- 1,2,4-oxadiazol-5-one | 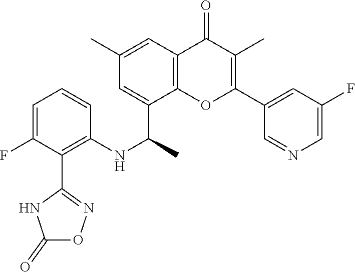 [SMILES: Cc1cc([C@@H](C)Nc2cccc(F)c2-c2noc(=O)[nH]2)c2oc(-c3cncc(F)c3)c(C)c(=O)c2c1] | 491 [M + H]⁺ |
| 36C | 3-[6-[[(1R)-1-[3,6-Dimethyl- 4-oxo-2-(2-pyridyl)chromen- 8-yl]ethyl]amino]-2,3- difluoro-phenyl]-4H-1,2,4- oxadiazol-5-one | 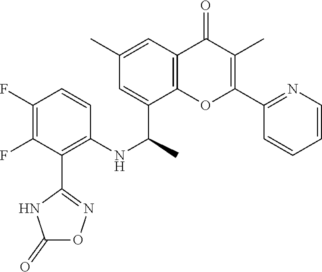 [SMILES: Cc1cc([C@@H](C)Nc2ccc(F)c(F)c2-c2noc(=O)[nH]2)c2oc(-c3ccccn3)c(C)c(=O)c2c1] | 491 [M + H]⁺ |
| 37C | 3-[6-[[(1R)-1-[3,6-Dimethyl- 2-(1-methylpyrazol-4-yl)-4- oxo-chromen-8- yl]ethyl]amino]-2,3-difluoro- phenyl]-4H-1,2,4-oxadiazol- 5-one | 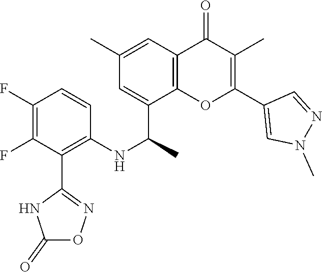 [SMILES: Cc1cc([C@@H](C)Nc2ccc(F)c(F)c2-c2noc(=O)[nH]2)c2oc(-c3cnn(C)c3)c(C)c(=O)c2c1] | 494 [M + H]⁺ |
| 38C | 3-[6-Chloro-3-[[(1R)-1-[2- (2,6-difluorophenyl)-3,6- dimethyl-4-oxo-chromen-8- yl]ethyl]amino]-2-pyridyl]- 4H-1,2,4-oxadiazol-5-one | 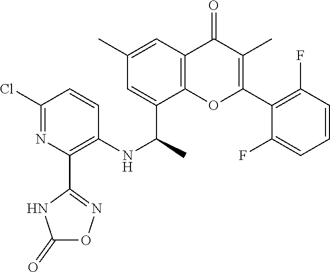 [SMILES: Cc1cc([C@@H](C)Nc2ccc(Cl)nc2-c2noc(=O)[nH]2)c2oc(-c3c(F)cccc3F)c(C)c(=O)c2c1] | 525 [M + H]⁺ |
| 39C | 3-[6-[[(1R)-1-[3,6-Dimethyl- 2-(2-methylindazol-5-yl)-4- oxo-chromen-8- yl]ethyl]amino]-2,3-difluoro- phenyl]-4H-1,2,4-oxadiazol- 5-one [HYW-00001-835] | 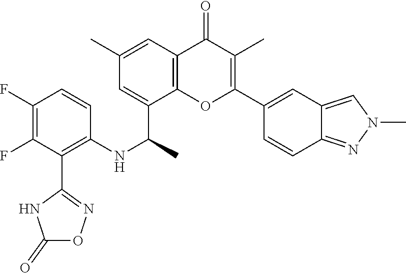 [SMILES: Cc1cc([C@@H](C)Nc2ccc(F)c(F)c2-c2noc(=O)[nH]2)c2oc(-c3ccc4nn(C)cc4c3)c(C)c(=O)c2c1] | 544 [M + H]⁺ |
| 40C | 3-[6-Chloro-3-[(1R)-1-[3,6- dimethyl-2-(2- methylpyrazolo[3,4- b]pyridin-5-yl)-4-oxo- chromen-8-yl]ethyl]amino]-2- pyridyl]-4H-1,2,4-oxadiazol- 5-one [NAW-00001-427] | 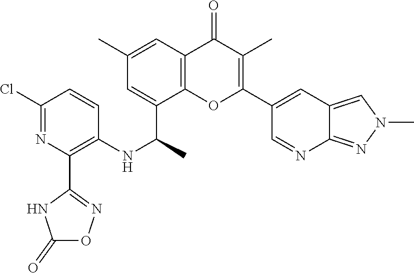 [SMILES: Cc1cc([C@@H](C)Nc2ccc(Cl)nc2-c2noc(=O)[nH]2)c2oc(-c3cnc4nn(C)cc4c3)c(C)c(=O)c2c1] | 544 [M + H]⁺ |
| 41C | (Z)-2-[8-[(1R)-1-[[6-Chloro- 2-(5-oxo-4H-1,2,4-oxadiazol- 3-yl)-3-pyridyl]amino]ethyl]- 6-methyl-4-oxo-2-(3- pyridyl)chromen-3-yl]-3- hydroxy-prop-2-enenitrile | 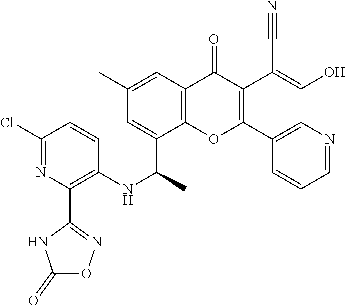 [SMILES: Cc1cc([C@@H](C)Nc2ccc(Cl)nc2-c2noc(=O)[nH]2)c2oc(-c3cccnc3)c(/C(C#N)=C/O)c(=O)c2c1] | 543 [M + H]⁺ |
| 42C | 3-[6-Chloro-3-[[(1R)-1-[2-(4- fluoro-2-pyridyl)-3,6- dimethyl-4-oxo-chromen-8- yl]ethyl]amino]-2-pyridyl]- 4H-1,2,4-oxadiazol-5-one | 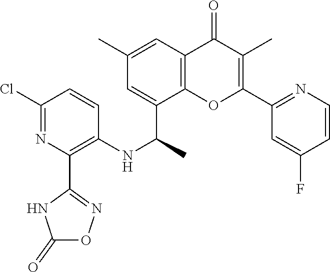 [SMILES: Cc1cc([C@@H](C)Nc2ccc(Cl)nc2-c2noc(=O)[nH]2)c2oc(-c3cc(F)ccn3)c(C)c(=O)c2c1] | 508 [M + H]⁺ |
| 43C | 3-[6-Chloro-3-[(1R)-1-[2- (6,7-dihydro-4H- pyrazolo[5,1-c][1,4]oxazin-3- yl)-3,6-dimethyl-4-oxo- chromen-8-yl]ethyl]amino]-2- pyridyl]-4H-1,2,4-oxadiazol- 5-one | 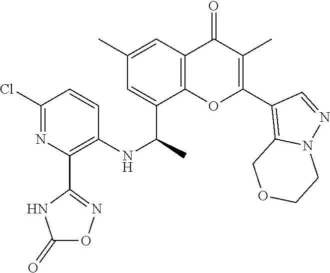 [SMILES: Cc1cc([C@@H](C)Nc2ccc(Cl)nc2-c2noc(=O)[nH]2)c2oc(-c3cnn4c3COCC4)c(C)c(=O)c2c1] | 535 [M + H]⁺ |
| 44C | 3-[6-Chloro-3-[[(1R)-1-[3,6- dimethyl-2-[1-(oxetan-3- yl)pyrazol-4-yl]-4-oxo- chromen-8-yl]ethyl]amino]-2- pyridyl]-4H-1,2,4-oxadiazol- 5-one | 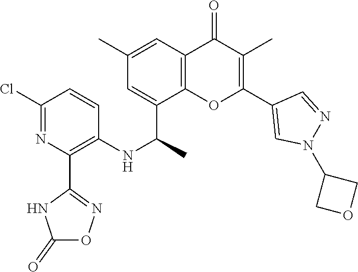 [SMILES: Cc1cc([C@@H](C)Nc2ccc(Cl)nc2-c2noc(=O)[nH]2)c2oc(-c3cnn(C4COC4)c3)c(C)c(=O)c2c1] | 535 [M + H]⁺ |
| 45C | 3-[6-Chloro-3-[(1R)-1-[3,6- dimethyl-2-[1-[(3- methyloxetan-3- yl)methyl]pyrazol-4-yl]-4- oxo-chromen-8- yl]ethyl]amino]-2-pyridyl]- 4H-1,2,4-oxadiazol-5-one | 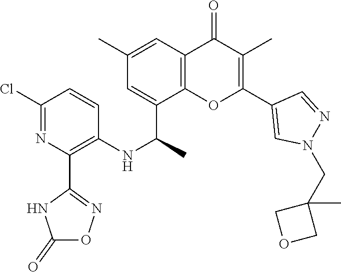 [SMILES: Cc1cc([C@@H](C)Nc2ccc(Cl)nc2-c2noc(=O)[nH]2)c2oc(-c3cnn(CC4(C)COC4)c3)c(C)c(=O)c2c1] | 563 [M + H]⁺ |
| 46C | 3-[6-Chloro-3-[(1R)-1-(3,6- dimethyl-4-oxo-2- pyrazolo[1,5-a]pyrimidin-3- yl-chromen-8- yl)ethyl]amino]-2-pyridyl]- 4H-1,2,4-oxadiazol-5-one | 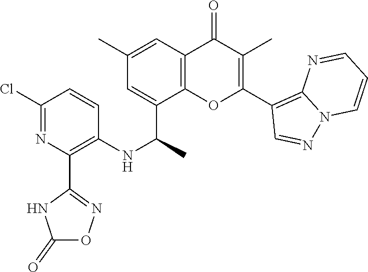 [SMILES: Cc1cc([C@@H](C)Nc2ccc(Cl)nc2-c2noc(=O)[nH]2)c2oc(-c3cnn4cccnc34)c(C)c(=O)c2c1] | 530 [M + H]⁺ |
| 47C | 3-[6-Chloro-3-[[(1R)-1-[3,6- dimethyl-4-oxo-2-[3- (trifluoromethyl)-1- bicyclo[1.1.1]pentanyl]chromen- 8-yl]ethyl]amino]-2- pyridyl]-4H-1,2,4-oxadiazol- 5-one | 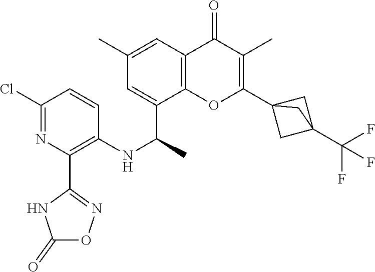 [SMILES: Cc1cc([C@@H](C)Nc2ccc(Cl)nc2-c2noc(=O)[nH]2)c2oc(C34CC(C(F)(F)F)(C3)C4)c(C)c(=O)c2c1] | 547 [M + H]⁺ |
| 48C | 3-[6-Chloro-3-[1-[6-fluoro-3- methyl-2-(1-methylpyrazol-4- yl)-4-oxo-chromen-8- ylethylamino]-2-pyridyl]-4H- 1,2,4-oxadiazol-5-one, Isomer 1 [SAL-00002-007] | 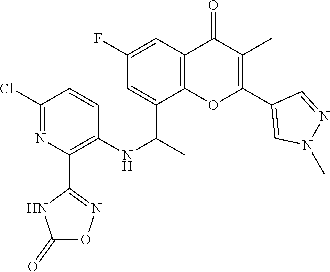 [SMILES: Cc1c(-c2cnn(C)c2)oc2c(C(C)Nc3ccc(Cl)nc3-c3noc(=O)[nH]3)cc(F)cc2c1=O] | 497 [M + H]⁺ |
| 49C | 3-[6-Chloro-3-[(1R)-1-(2- cyclopropyl-3,6-dimethyl-4- oxo-chromen-8- yl)ethyl]amino]-2-pyridyl]- 4H-1,2,4-oxadiazol-5-one | 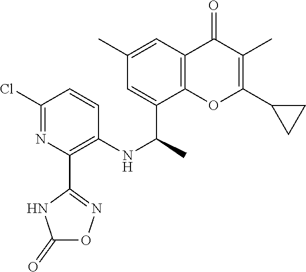 [SMILES: Cc1cc([C@@H](C)Nc2ccc(Cl)nc2-c2noc(=O)[nH]2)c2oc(C3CC3)c(C)c(=O)c2c1] | 453 [M + H]⁺ |

### Example 50C: 3-[6-Chloro-3-[[(1R)-1-[2-[1-(2-hydroxyethyl)pyrazol-4-yl]-3,6-dimethyl-4-oxo-chromen-8-yl]ethyl]amino]-2-pyridyl]-4H-1,2,4-oxadiazol-5-one

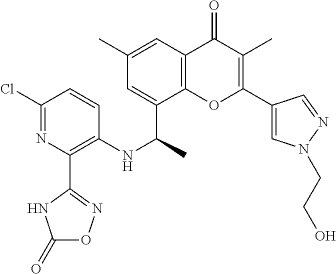 [SMILES: Cc1cc([C@@H](C)Nc2ccc(Cl)nc2-c2noc(=O)[nH]2)c2oc(-c3cnn(CCO)c3)c(C)c(=O)c2c1]

A solution of 3-[3-[[(1R)-1-[2-[1-[2-[tert-butyl(dimethyl)silyl]oxyethyl]pyrazol-4-yl]-3,6-dimethyl-4-oxo-chromen-8-yl]ethyl]amino]-6-chloro-2-pyridyl]-4H-1,2,4-oxadiazol-5-one (98.4 mg, 0.15 mmol) in THF (3 mL) was cooled in an ice bath and treated with tetrabutylammonium fluoride (1M in THF, 48.5 mg, 0.19 mmol). The reaction was stirred at 0° C. for 1 h. The reaction was concentrated under reduced pressure and the residue purified by reversed phase chromatography on C18 eluted with 0% to 100% ACN in water (with 0.1% formic acid). The resulting material was repurified by preparative TLC eluted with 4% MeOH in DCM and 8% MeOH in DCM to give the title compound (18.2 mg, 23%) as a white solid. ES/MS m/z 523 (M+H).

The following compound in Table 60 was made in a similar way as described for 3-[6-chloro-3-[[(1R)-1-[2-[1-(2-hydroxyethyl)pyrazol-4-yl]-3,6-dimethyl-4-oxo-chromen-8-yl]ethyl]amino]-2-pyridyl]-4H-1,2,4-oxadiazol-5-one. Various methods were used to purify the compounds, which would be apparent to one skilled in the art.

TABLE 60

| Example | | | MS ES+ |
|---|---|---|---|
| # | Chemical Name | Structure | m/z |
| 51C | 8-[(1R)-1-[[6-Chloro-2-(3- hydroxyazetidin-1-yl)-3- pyridyl]amino]ethyl]-3,6- dimethyl-2-(3- pyridyl)chromen-4-one | 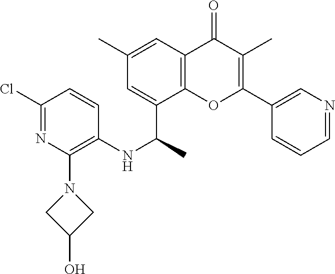 [SMILES: Cc1cc([C@@H](C)Nc2ccc(Cl)nc2N2CC(O)C2)c2oc(-c3cccnc3)c(C)c(=O)c2c1] | 477 [M + H]⁺ |

### Example 52C: 3-[6-Chloro-3-[[(1R)-1-[3,6-dimethyl-4-oxo-2-(6-oxo-1H-pyridin-2-yl)chromen-8-yl]ethyl]amino]-2-pyridyl]-4H-1,2,4-oxadiazol-5-one

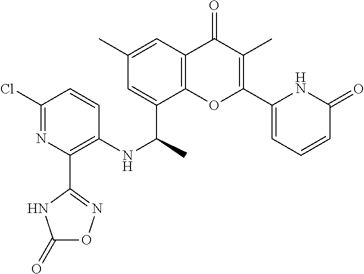 [SMILES: Cc1cc([C@@H](C)Nc2ccc(Cl)nc2-c2noc(=O)[nH]2)c2oc(-c3cccc(=O)[nH]3)c(C)c(=O)c2c1]

A solution of 3-[6-chloro-3-[[(1R)-1-[2-(6-methoxy-2-pyridyl)-3,6-dimethyl-4-oxo-chromen-8-yl]ethyl]amino]-2-pyridyl]-4H-1,2,4-oxadiazol-5-one (70 mg, 0.13 mmol) in DMF (1 mL) was treated with p-toluenesulfonic acid monohydrate (130 mg, 0.67 mmol) and lithium chloride (29 mg, 0.67 mmol). The reaction was sealed and heated at 120° C. for 40 min in a microwave. The reaction was allowed to cool to rt, concentrated under reduced pressure, and the residue purified by reversed phase chromatography on a CSH column eluted with 10% to 44% ACN in 10 mM aqueous NH₄HCO₃ (with 5% MeOH). The resulting residue was triturated with MeOH/MTBE resulting in the title compound (3.8 mg, 6%) being removed by filtration. ES/MS m/z 506 (M+H).

### Example 156A: 3,6-Dimethyl-2-phenyl-8-[(1R)-1-[[2-(5-thioxo-4H-1,2,4-oxadiazol-3-yl)-3-pyridyl]amino]ethyl]chromen-4-one

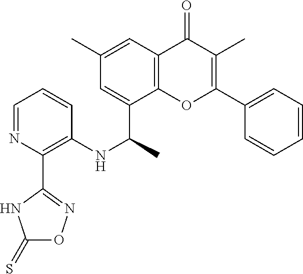 [SMILES: Cc1cc([C@@H](C)Nc2cccnc2-c2noc(=S)[nH]2)c2oc(-c3ccccc3)c(C)c(=O)c2c1]

A solution of 3-[[(1R)-1-(3,6-dimethyl-4-oxo-2-phenyl-chromen-8-yl)ethyl]amino]-N′-hydroxy-pyridine-2-carboxamidine (0.13 g, 0.30 mmol) in ACN (2 mL) was treated with DBU (0.09 g, 0.61 mmol) and N,N′-thiocarbonyldiimidazole (0.08 g, 0.46 mmol). The reaction was sealed and stirred at 80° C. for 3 h. The reaction was concentrated and purified by reversed phase C18 flash chromatography eluted with 0% to 100% ACN in 0.1% aqueous formic acid. Fractions containing the product were combined, washed with saturated aqueous sodium chloride, and extracted with IPA/CHCl₃ (1:3). The organics were collected, dried over MgSO₄, filtered, and concentrated. The residue was recrystallized from DCM/hexanes to give the title compound (74 mg, 52%) as a white solid. MS ES+ m/z 471 [M+H]⁺.

The following compound in Table 61 was made in a similar way as described for 3,6-dimethyl-2-phenyl-8-[(1R)-1-[[2-(5-thioxo-4H-1,2,4-oxadiazol-3-yl)-3-pyridyl]amino]ethyl]chromen-4-one.

TABLE 61

| Example | | | MS ES+ |
|---|---|---|---|
| # | Chemical Name | Structure | m/z |
| 157A | 8-[(1R)-1-[3,4- Difluoro-2-(5-thioxo- 4H-1,2,4-oxadiazol-3- yl)anilino]ethyl]-3,6- dimethyl-2-phenyl- chromen-4-one | 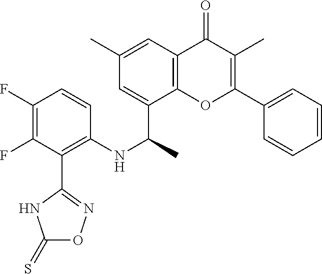 [SMILES: Cc1cc([C@@H](C)Nc2ccc(F)c(F)c2-c2noc(=S)[nH]2)c2oc(-c3ccccc3)c(C)c(=O)c2c1] | 506 [M + H]⁺ |

### Example 158A: 3-[3-[[(1R)-1-(3,6-Dimethyl-4-oxo-2-phenyl-chromen-8-yl)ethyl]amino]-2-pyridyl]-4-methyl-1,2,4-oxadiazol-5-one

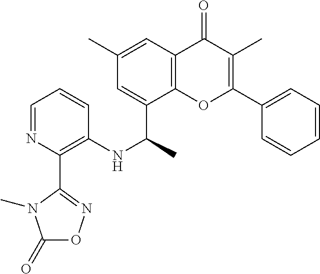 [SMILES: Cc1cc([C@@H](C)Nc2cccnc2-c2noc(=O)n2C)c2oc(-c3ccccc3)c(C)c(=O)c2c1]

A solution of 3-[3-[[(1R)-1-(3,6-dimethyl-4-oxo-2-phenyl-chromen-8-yl)ethyl]amino]-2-pyridyl]-4H-1,2,4-oxadiazol-5-one (0.10 g, 0.22 mmol) in 2 mL of DMF was treated with potassium carbonate (0.11 g, 0.77 mmol) and iodomethane (37.5 mg, 0.26 mmol) and the reaction allowed to stir at rt overnight. The reaction was concentrated and the residue purified by reversed phase chromatography eluted with 0% to 100% ACN in water (with 0.1% formic acid). Fractions containing the product were pooled, washed with saturated aqueous sodium chloride, and extracted with IPA/CHCl₃ (1:3). The organic layers were combined, dried over MgSO₄, filtered, and concentrated. The residue was crystallized from a mixture of DCM and hexanes to give the title compound (64 mg, 62%) as a white solid. MS ES+ m/z 469 [M+H]⁺.

### Example 43A: 3,6-Dimethyl-8-[(1R)-1-[2-(1,2,4-oxadiazol-3-yl)anilino]ethyl]-2-phenyl-chromen-4-one

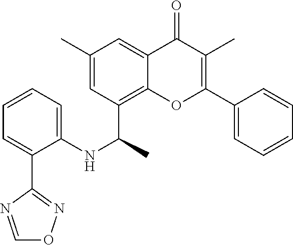 [SMILES: Cc1cc([C@@H](C)Nc2ccccc2-c2ncon2)c2oc(-c3ccccc3)c(C)c(=O)c2c1]

To a solution of 2-[[(1R)-1-(3,6-dimethyl-4-oxo-2-phenyl-chromen-8-yl)ethyl]amino]-N′-hydroxy-benzamidine (0.100 g, 0.234 mmol) in triethyl orthoformate (2 mL, 10 mmol) was added TFA (0.005 mL). The reaction was sealed and heated in a microwave reactor at 120° C. for 1 h. The mixture was diluted with EtOAc and washed with saturated aqueous NaHCO₃ and saturated aqueous sodium chloride. The organic layer was dried over Na₂SO₄, filtered, and concentrated. The residue was purified by silica gel chromatography eluted with a gradient of 0% to 100% EtOAc in heptane. Fractions with the desired product were combined and concentrated and the residue was re-purified by reverse phase chromatography eluted with a gradient of 0% to 100% ACN in aqueous formic acid (0.1%). Fractions containing the desired product were combined, washed with saturated aqueous sodium chloride, and extracted with EtOAc. The organic layer was dried over Na₂SO₄, filtered, and concentrated to give the title compound (0.038 g, 37%) as a white solid. MS ES+ m/z 438 [M+H]⁺.

The following compound in Table 62 was made in a similar way as described for 3,6-dimethyl-8-[(1R)-1-[2-(1,2,4-oxadiazol-3-yl)anilino]ethyl]-2-phenyl-chromen-4-one. Various methods were used to purify the compound, which would be apparent to one skilled in the art.

TABLE 62

| Example | | | MS ES+ |
|---|---|---|---|
| # | Chemical Name | Structure | m/z |
| 53C | 3,6-Dimethyl-8-[(1R)-1-[[2- (1,2,4-oxadiazol-3-yl)-3- pyridyl]amino]ethyl]-2- phenyl-chromen-4-one | 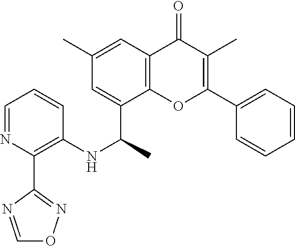 [SMILES: Cc1cc([C@@H](C)Nc2cccnc2-c2ncon2)c2oc(-c3ccccc3)c(C)c(=O)c2c1] | 439 [M + H]⁺ |

### Example 54C: 8-[(1R)-1-[2-[2-[Dimethyl(oxo)-lambda6-sulfanylidene]acetyl]anilino]ethyl]-3,6-dimethyl-2-phenyl-chromen-4-one

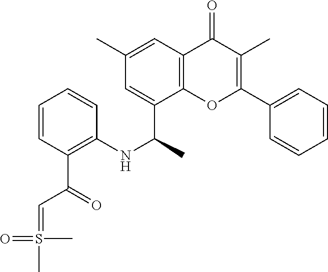 [SMILES: Cc1cc([C@@H](C)Nc2ccccc2C(=O)C=S(C)(C)=O)c2oc(-c3ccccc3)c(C)c(=O)c2c1]

Synthesized according to the method described for Intermediate 307C.

### Example 55C: 3-[2-[[(1R)-1-(3,6-Dimethyl-4-oxo-2-phenyl-chromen-8-yl)ethyl]amino]phenyl]-3-oxo-propanenitrile

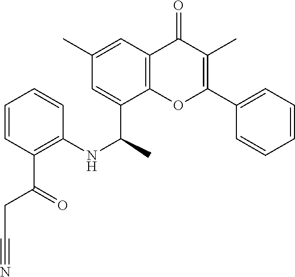 [SMILES: Cc1cc([C@@H](C)Nc2ccccc2C(=O)CC#N)c2oc(-c3ccccc3)c(C)c(=O)c2c1]

A vial was charged with 8-[(1R)-1-[2-(2-chloroacetyl)anilino]ethyl]-3,6-dimethyl-2-phenyl-chromen-4-one (17.6 mg, 0.04 mmol), sodium cyanide (11.6 mg, 0.24 mmol), and DMF (0.4 mL) at rt. The reaction was stirred for 30 min. The crude reaction was purified by reversed phase chromatography on C18 eluted with 0% to 100% ACN in 10 mM aqueous NH₄HCO₃ (with 5% MeOH) to give the title compound (16.2 mg, 94%) as a light yellow powder. ES/MS m/z 437 (M+H).

### Example 44A: 2-[[(1R)-1-(3,6-Dimethyl-4-oxo-2-phenyl-chromen-8-yl)ethyl]amino]benzamide

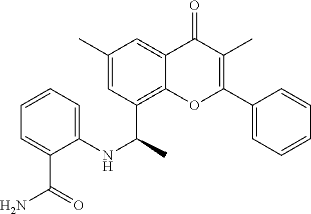 [SMILES: Cc1cc([C@@H](C)Nc2ccccc2C(N)=O)c2oc(-c3ccccc3)c(C)c(=O)c2c1]

In an oven-dried round bottom flask, 2-[[(1R)-1-(3,6-dimethyl-4-oxo-2-phenyl-chromen-8-yl)ethyl]amino]benzoic acid (0.200 g, 0.484 mmol) was dissolved in dry THF (2 mL) and thionyl chloride (2 M solution in DCM, 0.36 mL, 0.73 mmol) was added at 0° C. The reaction was stirred at rt for 45 min, cooled to 0° C., and then ice-cold aqueous ammonium hydroxide (5 wt %) was added in one portion. The reaction was removed from the ice bath and stirred for 15 min. The mixture was extracted 3× with DCM. The combined organic layers were dried over Na₂SO₄ and concentrated. The residue was purified by reverse phase chromatography eluted with a gradient of 0 to 100% ACN in aqueous NH₄HCO₃ (10 mM+5% MeOH) to give the title compound (0.160 g, 80%). MS ES+ m/z 413 [M+H]⁺.

The following compound in Table 63 was made in a similar way as described for 3-[[(1R)-1-(3,6-dimethyl-4-oxo-2-phenyl-chromen-8-yl)ethyl]amino]pyridine-2-carbohydrazide.

TABLE 63

| Example | | | MS ES+ |
|---|---|---|---|
| # | Chemical Name | Structure | m/z |
| 45A | 2-[[(1R)-1-(3,6- Dimethyl-4-oxo-2- phenyl-chromen-8- yl)ethyl]amino] benzohydrazide | 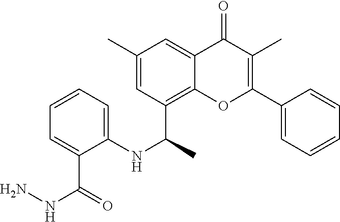 [SMILES: Cc1cc([C@@H](C)Nc2ccccc2C(=O)NN)c2oc(-c3ccccc3)c(C)c(=O)c2c1] | 428 [M + H]⁺ |

### Example 46A: 2-[[(1R)-1-(3,6-Dimethyl-4-oxo-2-phenyl-chromen-8-yl)ethyl]amino]-N,N-dimethyl-benzamide

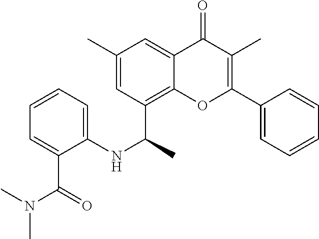 [SMILES: Cc1cc([C@@H](C)Nc2ccccc2C(=O)N(C)C)c2oc(-c3ccccc3)c(C)c(=O)c2c1]

To a stirred suspension of 2-[[(1R)-1-(3,6-dimethyl-4-oxo-2-phenyl-chromen-8-yl)ethyl]amino]benzoic acid (0.200 g, 0.484 mmol) in dry DCM (30 mL) was added thionyl chloride (2 M solution in DCM, 0.36 mL, 0.73 mmol) at 0° C., then the reaction was stirred for 1 h at rt. Triethylamine (0.20 mL, 1.45 mmol) was added followed by the addition of dimethylamine (10 wt %, 0.220 g, 0.484 mmol). The reaction was stirred at rt for 4 h, then diluted with water, followed by addition of 1N aqueous NaOH to adjust the pH>7. The aqueous layer was extracted 3× with DCM. The organic extracts were dried over Na₂SO₄ and concentrated. The residue was purified by reverse phase chromatography eluted with a gradient of 0 to 100% ACN in aqueous NH₄HCO₃ (10 mM+5% MeOH) to give the title compound (0.178 g, 84%) as a white solid. MS ES+ m/z 441 [M+H]⁺.

The following compounds in Table 64 were made in a similar way as described for 2-[[(1R)-1-(3,6-dimethyl-4-oxo-2-phenyl-chromen-8-yl)ethyl]amino]-N,N-dimethyl-benzamide. Various methods were used to purify the compounds, which would be apparent to one skilled in the art.

TABLE 64

| Example | | | MS ES+ |
|---|---|---|---|
| # | Chemical Name | Structure | m/z |
| 47A | 2-[[(1R)-1-(3,6- Dimethyl-4-oxo-2- phenyl-chromen-8- yl)ethyl]amino]-N,N- diethyl-benzamide | 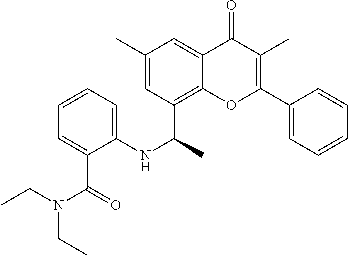 [SMILES: CCN(CC)C(=O)c1ccccc1N[C@H](C)c1cc(C)cc2c(=O)c(C)c(-c3ccccc3)oc12] | 469 [M + H]⁺ |
| 48A | 1-[3-[[(1R)-1-(3,6- Dimethyl-4-oxo-2- phenyl-chromen-8- yl)ethyl]amino] pyridine-2- carbonyl]pyrrolidine-3- carbonitrile | 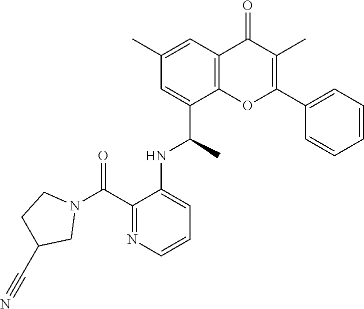 [SMILES: Cc1cc([C@@H](C)Nc2cccnc2C(=O)N2CCC(C#N)C2)c2oc(-c3ccccc3)c(C)c(=O)c2c1] | 493 [M + H]⁺ |

### Example 49A: N-Benzyl-2-[[(1R)-1-(3,6-dimethyl-4-oxo-2-phenyl-chromen-8-yl)ethyl]amino]benzamide

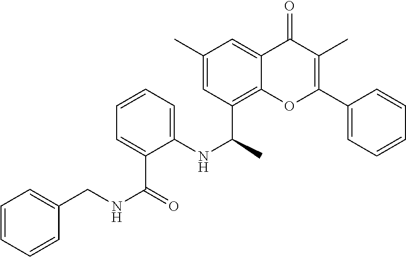 [SMILES: Cc1cc([C@@H](C)Nc2ccccc2C(=O)NCc2ccccc2)c2oc(-c3ccccc3)c(C)c(=O)c2c1]

To a solution of 2-[[(1R)-1-(3,6-dimethyl-4-oxo-2-phenyl-chromen-8-yl)ethyl]amino]benzoic acid (0.200 g, 0.484 mmol) in DCM was added di(1H-imidazol-1-yl)methanone (0.078 g, 0.484 mmol). The reaction was stirred for 1 h at rt and benzylamine (0.053 mL, 0.48 mmol) was added. The reaction was stirred for 6 h at rt, then diluted with DCM and water. The aqueous layer was extracted 3×. The crude product was purified by reverse phase chromatography eluted with a gradient of ACN in aqueous NH₄HCO₃ (10 mM, plus 500 MeOH) to give the title compound (0.23 g, 950%). MS ES+ m/z 503 [M+H]⁺.

The following compounds in Table 65 were made in a similar way as described for N-benzyl-2-[[(1R)-1-(3,6-dimethyl-4-oxo-2-phenyl-chromen-8-yl)ethyl]amino]benzamide. Various methods were used to purify the compounds, which would be apparent to one skilled in the art.

TABLE 65

| Example | | | MS ES+ |
|---|---|---|---|
| # | Chemical Name | Structure | m/z |
| 50A | 2-[[(1R)-1-(3,6- Dimethyl-4-oxo-2- phenyl-chromen-8- yl)ethyl]amino]benzene carbohydroxamic acid | 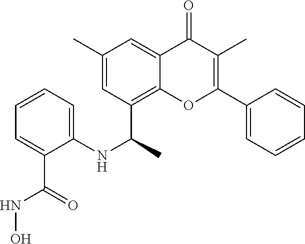 [SMILES: Cc1cc([C@@H](C)Nc2ccccc2C(=O)NO)c2oc(-c3ccccc3)c(C)c(=O)c2c1] | 429 [M + H]⁺ |
| 51A | 2-[(1R)-1-(3,6- Dimethyl-4-oxo-2- phenyl-chromen-8- yl)ethyl]amino]-N- methyl-benzamide | 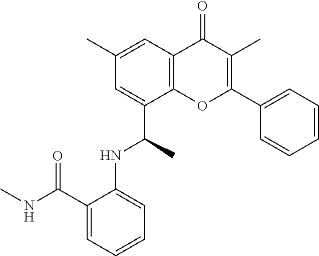 [SMILES: CNC(=O)c1ccccc1N[C@H](C)c1cc(C)cc2c(=O)c(C)c(-c3ccccc3)oc12] | 427 [M + H]⁺ |
| 52A | 2-[[(1R)-1-(3,6- Dimethyl-4-oxo-2- phenyl-chromen-8- yl)ethyl]amino]-N- phenyl-benzamide | 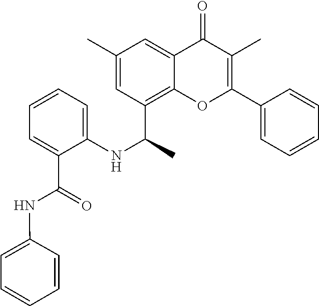 [SMILES: Cc1cc([C@@H](C)Nc2ccccc2C(=O)Nc2ccccc2)c2oc(-c3ccccc3)c(C)c(=O)c2c1] | 489 [M + H]⁺ |
| 53A | 3-[[(1R)-1-(3,6- Dimethyl-4-oxo-2- phenyl-chromen-8- yl)ethyl]amino]-N- (tetrahydrofuran-2- ylmethyl)pyridine-2- carboxamide | 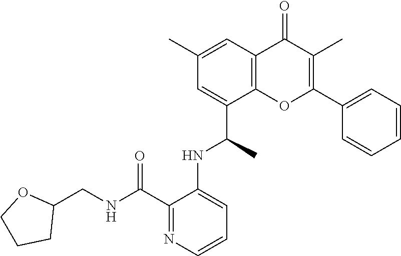 [SMILES: Cc1cc([C@@H](C)Nc2cccnc2C(=O)NCC2CCCO2)c2oc(-c3ccccc3)c(C)c(=O)c2c1] | 498 [M + H]⁺ |
| 54A | 3-[[(1R)-1-(3,6- Dimethyl-4-oxo-2- phenyl-chromen-8- yl)ethyl]amino]-N- (oxetan-3- ylmethyl)pyridine-2- carboxamide | 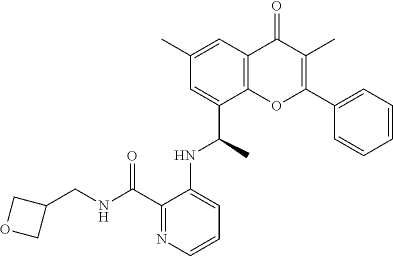 [SMILES: Cc1cc([C@@H](C)Nc2cccnc2C(=O)NCC2COC2)c2oc(-c3ccccc3)c(C)c(=O)c2c1] | 484 [M + H]⁺ |
| 55A | 3-[[(1R)-1-(3,6- Dimethyl-4-oxo-2- phenyl-chromen-8- yl)ethyl]amino]-N-(2- hydroxyethyl)pyridine- 2-carboxamide | 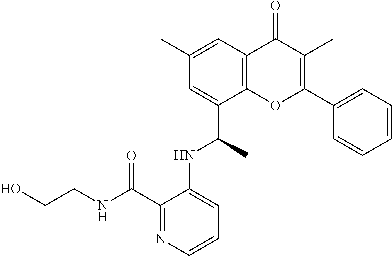 [SMILES: Cc1cc([C@@H](C)Nc2cccnc2C(=O)NCCO)c2oc(-c3ccccc3)c(C)c(=O)c2c1] | 458 [M + H]⁺ |
| 56A | 3-[(1R)-1-(3,6- Dimethyl-4-oxo-2- phenyl-chromen-8- yl)ethyl]amino]-N-(2- methoxyethyl)pyridine- 2-carboxamide | 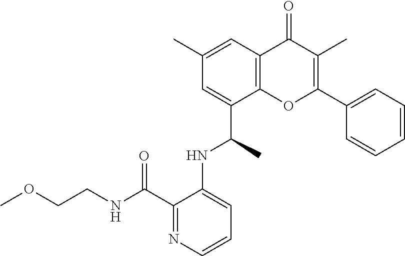 [SMILES: COCCNC(=O)c1ncccc1N[C@H](C)c1cc(C)cc2c(=O)c(C)c(-c3ccccc3)oc12] | 472 [M + H]⁺ |

### Example 159A: 6-Chloro-3-[[(1R)-1-(3,6-dimethyl-4-oxo-2-phenyl-chromen-8-yl)ethyl]amino]pyridine-2-carboxamide

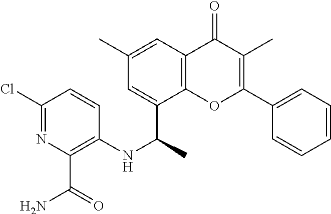 [SMILES: Cc1cc([C@@H](C)Nc2ccc(Cl)nc2C(N)=O)c2oc(-c3ccccc3)c(C)c(=O)c2c1]

Combined 6-chloro-3-[[(1R)-1-(3,6-dimethyl-4-oxo-2-phenyl-chromen-8-yl)ethyl]amino]pyridine-2-carboxylic acid (0.10 g, 0.22 mmol), DIPEA (0.17 g, 1.34 mmol), 2,4,6-tripropyl-1,3,5-trioxa-2,4,6-triphosphinane-2,4,6-trioxide (0.85 g, 1.34 mmol), and ammonia (0.5 M, 0.23 mg, 1.34 mmol) in DMA (0.5 mL) and the reaction stirred at 25° C. for 12 h. Reaction was directly purified by reversed phase chromatography eluted with 10% to 100% ACN in aqueous 0.1 mM NH₄HCO₃ with 5% MeOH to give the title compound (22.3 mg, 22%). MS ES+ m/z 448 [M+H]⁺.

The following compounds in Table 66 were made in a similar way as described for 6-chloro-3-[[(1R)-1-(3,6-dimethyl-4-oxo-2-phenyl-chromen-8-yl)ethyl]amino]pyridine-2-carboxamide. Various methods were used to purify the compounds, which would be apparent to one skilled in the art.

TABLE 66

| Example | | | MS ES+ |
|---|---|---|---|
| # | Chemical Name | Structure | m/z |
| 160A | 6-Chloro-3-[[(1R)-1- [2-[1- (difluoromethyl)pyrazol- 4-yl]-3,6-dimethyl-4- oxo-chromen-8- yl]ethyl]amino]pyridine- 2-carboxamide | 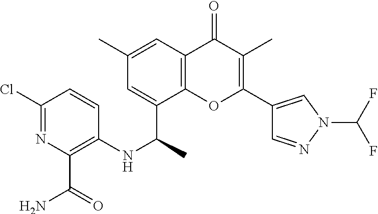 [SMILES: Cc1cc([C@@H](C)Nc2ccc(Cl)nc2C(N)=O)c2oc(-c3cnn(C(F)F)c3)c(C)c(=O)c2c1] | 488 [M + H]⁺ |
| 161A | 6-Chloro-3-[[(1R)-1- (3,6-dimethyl-4-oxo-2- phenyl-chromen-8- yl)ethyl]amino]pyridine- 2-carbohydroxamic acid | 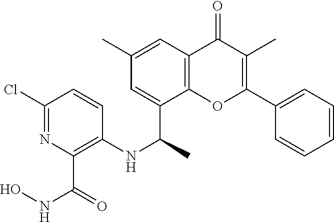 [SMILES: Cc1cc([C@@H](C)Nc2ccc(Cl)nc2C(=O)NO)c2oc(-c3ccccc3)c(C)c(=O)c2c1] | 464 [M + H]⁺ |
| 162A | 6-Chloro-3-[(1R)-1- (3,6-dimethyl-4-oxo-2- phenyl-chromen-8- yl)ethyl]amino]pyridine- 2-carbohydrazide | 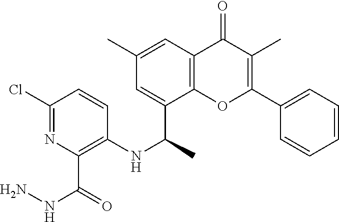 [SMILES: Cc1cc([C@@H](C)Nc2ccc(Cl)nc2C(=O)NN)c2oc(-c3ccccc3)c(C)c(=O)c2c1] | 463 [M + H]⁺ |
| 163A | 6-Chloro-3-[(1R)-1- (3,6-dimethyl-4-oxo-2- phenyl-chromen-8- yl)ethyl]amino]-N- methoxy-pyridine-2- carboxamide | 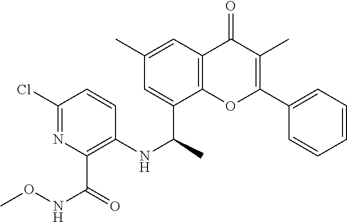 [SMILES: CONC(=O)c1nc(Cl)ccc1N[C@H](C)c1cc(C)cc2c(=O)c(C)c(-c3ccccc3)oc12] | 478 [M + H]⁺ |
| 164A¹ | 3-[[(1R)-1-(3,6- Dimethyl-4-oxo-2- phenyl-chromen-8- yl)ethyl]amino]pyridine- 2-carboxamide | 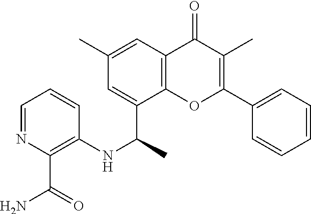 [SMILES: Cc1cc([C@@H](C)Nc2cccnc2C(N)=O)c2oc(-c3ccccc3)c(C)c(=O)c2c1] | 414 [M + H]⁺ |
| 165A | 6-Chloro-3-[[(1R)-1- [3,6-dimethyl-4-oxo-2- (2-pyridyl)chromen-8- yl]ethyl]amino]pyridine- 2-carboxamide | 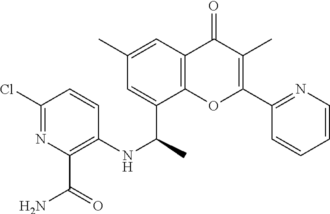 [SMILES: Cc1cc([C@@H](C)Nc2ccc(Cl)nc2C(N)=O)c2oc(-c3ccccn3)c(C)c(=O)c2c1] | 449 [M + H]⁺ |
| 166A | 6-Chloro-3-[[(1R)-1- (3,6-dimethyl-4-oxo-2- phenyl-chromen-8- yl)ethyl]amino]-N- methyl-pyridine-2- carboxamide | 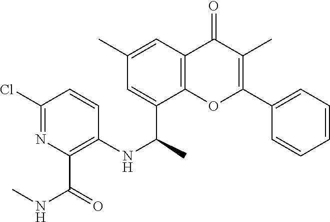 [SMILES: CNC(=O)c1nc(Cl)ccc1N[C@H](C)c1cc(C)cc2c(=O)c(C)c(-c3ccccc3)oc12] | 462 [M + H]⁺ |
| 167A | 6-Chloro-3-[(1R)-1- [3,6-dimethyl-4-oxo-2- (2-pyridyl)chromen-8- yl]ethyl]amino]-N- methoxy-pyridine-2- carboxamide | 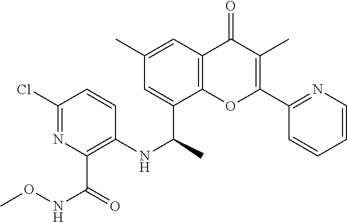 [SMILES: CONC(=O)c1nc(Cl)ccc1N[C@H](C)c1cc(C)cc2c(=O)c(C)c(-c3ccccn3)oc12] | 479 [M + H]⁺ |
| 168A | 6-Chloro-3-[[(1R)-1- [3,6-dimethyl-2-(2- methylimidazo[1,2- a]pyridin-6-yl)-4-oxo- chromen-8- yl]ethyl]amino]pyridine- 2-carboxamide | 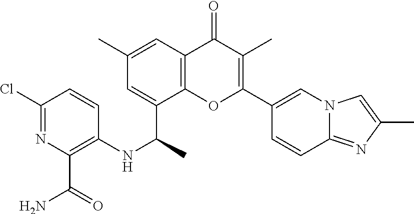 [SMILES: Cc1cc([C@@H](C)Nc2ccc(Cl)nc2C(N)=O)c2oc(-c3ccc4nc(C)cn4c3)c(C)c(=O)c2c1] | 503 [M + H]⁺ |
| 169A² | 3-[[(1R)-1-(3,6- Dimethyl-4-oxo-2- phenyl-chromen-8- yl)ethyl]amino]-N-[(5- hydroxy-1,3,4- oxadiazol-2- yl)methyl]pyridine-2- carboxamide | 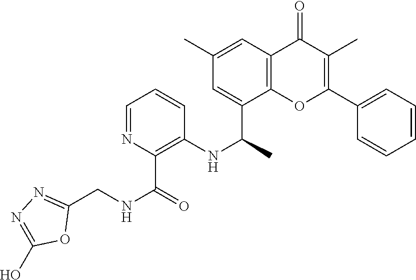 [SMILES: Cc1cc([C@@H](C)Nc2cccnc2C(=O)NCc2nnc(O)o2)c2oc(-c3ccccc3)c(C)c(=O)c2c1] | 512 [M + H]⁺ |
| 170A² | 3-[[(1R)-1-(3,6- Dimethyl-4-oxo-2- phenyl-chromen-8- yl)ethyl]amino]-N-[(1- methylpyrrol-3- yl)methyl]pyridine-2- carboxamide | 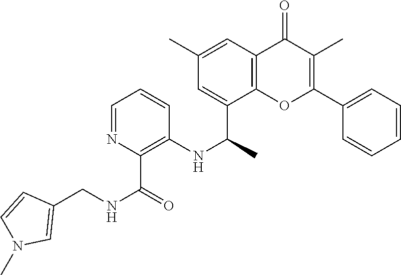 [SMILES: Cc1cc([C@@H](C)Nc2cccnc2C(=O)NCc2ccn(C)c2)c2oc(-c3ccccc3)c(C)c(=O)c2c1] | 507 [M + H]⁺ |
| 171A² | 3-[[(1R)-1-(3,6- Dimethyl-4-oxo-2- phenyl-chromen-8- yl)ethyl]amino]-N- ethyl-pyridine-2- carboxamide | 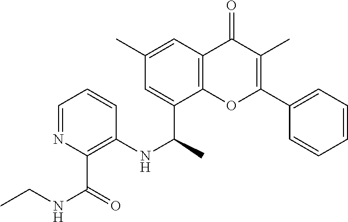 [SMILES: CCNC(=O)c1ncccc1N[C@H](C)c1cc(C)cc2c(=O)c(C)c(-c3ccccc3)oc12] | 442 [M + H]⁺ |
| 172A² | 3-[[(1R)-1-(3,6- Dimethyl-4-oxo-2- phenyl-chromen-8- yl)ethyl]amino]-N-(4- hydroxy-1- bicyclo[2.2.2]octanyl) pyridine-2-carboxamide | 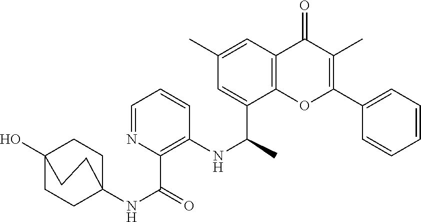 [SMILES: Cc1cc([C@@H](C)Nc2cccnc2C(=O)NC23CCC(O)(CC2)CC3)c2oc(-c3ccccc3)c(C)c(=O)c2c1] | 538 [M + H]⁺ |
| 173A² | 3-[[(1R)-1-(3,6- Dimethyl-4-oxo-2- phenyl-chromen-8- yl)ethyl]amino]-N- [[(3S)-tetrahydrofuran- 3-yl]methyl]pyridine-2- carboxamide | 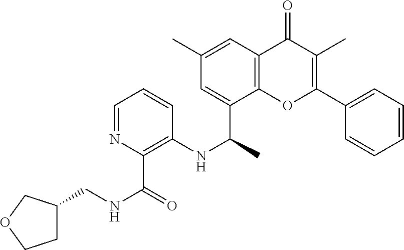 [SMILES: Cc1cc([C@@H](C)Nc2cccnc2C(=O)NC[C@@H]2CCOC2)c2oc(-c3ccccc3)c(C)c(=O)c2c1] | 498 [M + H]⁺ |
| 174A² | N- (Cyclopropylmethyl)- 3-[[(1R)-1-(3,6- dimethyl-4-oxo-2- phenyl-chromen-8- yl)ethyl]amino]pyridine- 2-carboxamide | 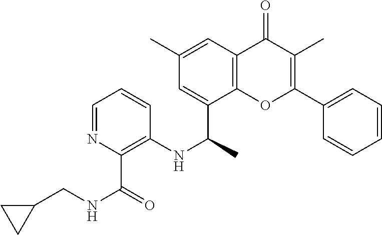 [SMILES: Cc1cc([C@@H](C)Nc2cccnc2C(=O)NCC2CC2)c2oc(-c3ccccc3)c(C)c(=O)c2c1] | 468 [M + H]⁺ |
| 175A² | 3-[[(1R)-1-(3,6- Dimethyl-4-oxo-2- phenyl-chromen-8- yl)ethyl]amino]-N-(2- pyridylmethyl)pyridine- 2-carboxamide | 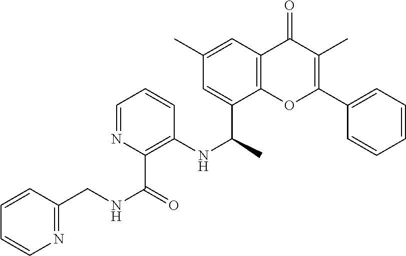 [SMILES: Cc1cc([C@@H](C)Nc2cccnc2C(=O)NCc2ccccn2)c2oc(-c3ccccc3)c(C)c(=O)c2c1] | 505 [M + H]⁺ |
| 176A² | N-Cyclopentyl-3- [[(1R)-1-(3,6-dimethyl- 4-oxo-2-phenyl- chromen-8- yl)ethyl]amino]pyridine- 2-carboxamide | 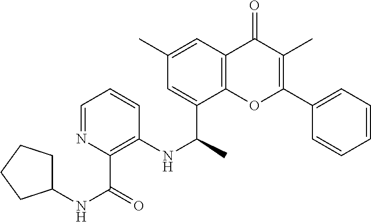 [SMILES: Cc1cc([C@@H](C)Nc2cccnc2C(=O)NC2CCCC2)c2oc(-c3ccccc3)c(C)c(=O)c2c1] | 482 [M + H]⁺ |
| 177A² | 3-[[(1R)-1-(3,6- Dimethyl-4-oxo-2- phenyl-chromen-8- yl)ethyl]amino]-N-(2- hydroxy-2-methyl- propyl)pyridine-2- carboxamide | 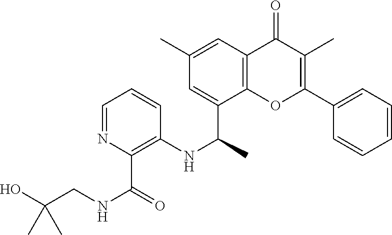 [SMILES: Cc1cc([C@@H](C)Nc2cccnc2C(=O)NCC(C)(C)O)c2oc(-c3ccccc3)c(C)c(=O)c2c1] | 486 [M + H]⁺ |
| 178A² | 3-[[(1R)-1-(3,6- Dimethyl-4-oxo-2- phenyl-chromen-8- yl)ethyl]amino]-N- [(3R)-tetrahydrofuran- 3-yl]pyridine-2- carboxamide | 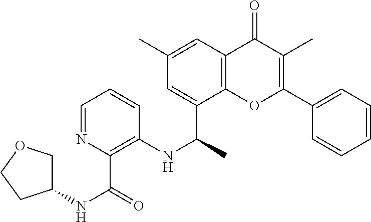 [SMILES: Cc1cc([C@@H](C)Nc2cccnc2C(=O)N[C@@H]2CCOC2)c2oc(-c3ccccc3)c(C)c(=O)c2c1] | 484 [M + H]⁺ |
| 179A² | 3-[[(1R)-1-(3,6- Dimethyl-4-oxo-2- phenyl-chromen-8- yl)ethyl]amino]-N- (oxetan-3-yl)pyridine- 2-carboxamide | 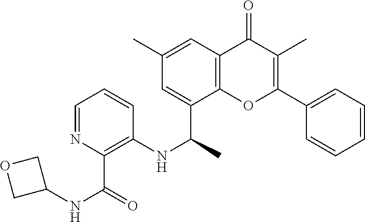 [SMILES: Cc1cc([C@@H](C)Nc2cccnc2C(=O)NC2COC2)c2oc(-c3ccccc3)c(C)c(=O)c2c1] | 470 [M + H]⁺ |
| 180A² | N-Cyclopropyl-3- [[(1R)-1-(3,6-dimethyl- 4-oxo-2-phenyl- chromen-8- yl)ethyl]amino]pyridine- 2-carboxamide | 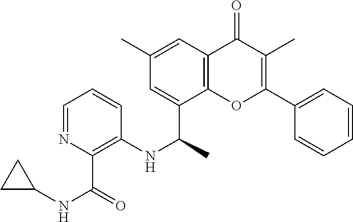 [SMILES: Cc1cc([C@@H](C)Nc2cccnc2C(=O)NC2CC2)c2oc(-c3ccccc3)c(C)c(=O)c2c1] | 454 [M + H]⁺ |
| 181A² | 3-[[(1R)-1-(3,6- Dimethyl-4-oxo-2- phenyl-chromen-8- yl)ethyl]amino]-N-(4- pyridylmethyl)pyridine- 2-carboxamide | 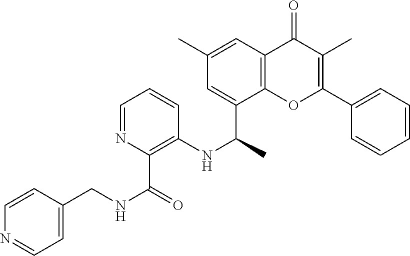 [SMILES: Cc1cc([C@@H](C)Nc2cccnc2C(=O)NCc2ccncc2)c2oc(-c3ccccc3)c(C)c(=O)c2c1] | 505 [M + H]⁺ |
| 182A² | 3-[[(1R)-1-(3,6- Dimethyl-4-oxo-2- phenyl-chromen-8- yl)ethyl]amino]-N- [(3S)-tetrahydrofuran- 3-yl]pyridine-2- carboxamide | 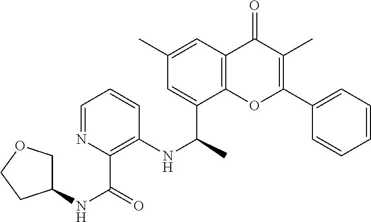 [SMILES: Cc1cc([C@@H](C)Nc2cccnc2C(=O)N[C@H]2CCOC2)c2oc(-c3ccccc3)c(C)c(=O)c2c1] | 484 [M + H]⁺ |
| 183A² | 3-[[(1R)-1-(3,6- Dimethyl-4-oxo-2- phenyl-chromen-8- yl)ethyl]amino]-N-(1H- indol-5- ylmethyl)pyridine-2- carboxamide | 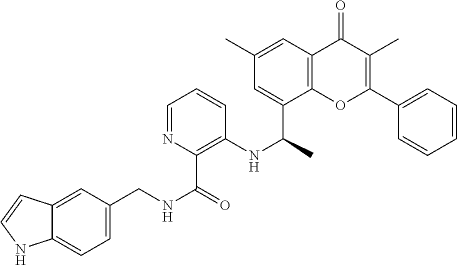 [SMILES: Cc1cc([C@@H](C)Nc2cccnc2C(=O)NCc2ccc3[nH]ccc3c2)c2oc(-c3ccccc3)c(C)c(=O)c2c1] | 543 [M + H]⁺ |
| 184A² | 3-[[(1R)-1-(3,6- Dimethyl-4-oxo-2- phenyl-chromen-8- yl)ethyl]amino]-N- [[(3R)-tetrahydrofuran- 3-yl]methyl]pyridine-2- carboxamide | 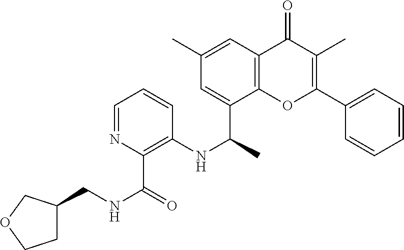 [SMILES: Cc1cc([C@@H](C)Nc2cccnc2C(=O)NC[C@H]2CCOC2)c2oc(-c3ccccc3)c(C)c(=O)c2c1] | 498 [M + H]⁺ |
| 185A² | 3-[(1R)-1-(3,6- Dimethyl-4-oxo-2- phenyl-chromen-8- yl)ethyl]amino]-N-(3- pyridylmethyl)pyridine- 2-carboxamide | 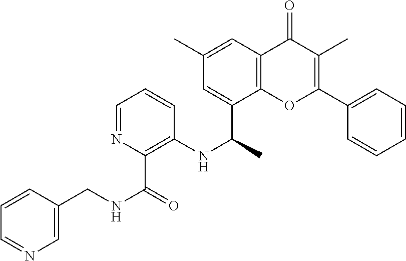 [SMILES: Cc1cc([C@@H](C)Nc2cccnc2C(=O)NCc2cccnc2)c2oc(-c3ccccc3)c(C)c(=O)c2c1] | 505 [M + H]⁺ |
| 186A² | 3-[[(1R)-1-(3,6- Dimethyl-4-oxo-2- phenyl-chromen-8- yl)ethyl]amino]-N- [(1R)-1- phenylethyl]pyridine-2- carboxamide | 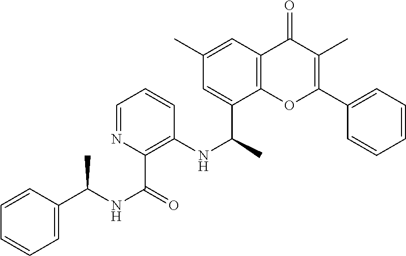 [SMILES: Cc1cc([C@@H](C)Nc2cccnc2C(=O)N[C@H](C)c2ccccc2)c2oc(-c3ccccc3)c(C)c(=O)c2c1] | 518 [M + H]⁺ |
| 187A² | 3-[[(1R)-1-[3,6- Dimethyl-4-oxo-2-(3- pyridyl)chromen-8- yl]ethyl]amino]-N- ethyl-pyridine-2- carboxamide | 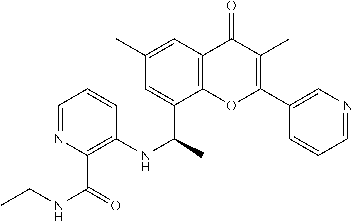 [SMILES: CCNC(=O)c1ncccc1N[C@H](C)c1cc(C)cc2c(=O)c(C)c(-c3cccnc3)oc12] | 443 [M + H]⁺ |
| 188A² | 6-Chloro-3-[[(1R)-1- [3,6-dimethyl-4-oxo-2- (3-pyridyl)chromen-8- yl]ethyl]amino]-N- ethyl-pyridine-2- carboxamide | 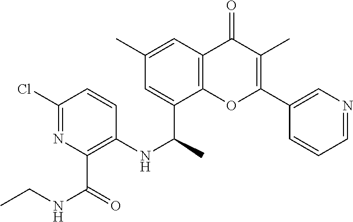 [SMILES: CCNC(=O)c1nc(Cl)ccc1N[C@H](C)c1cc(C)cc2c(=O)c(C)c(-c3cccnc3)oc12] | 477 [M + H]⁺ |
| 56C | 3-[[(1R)-1-[3,6- Dimethyl-4-oxo-2-(3- pyridyl)chromen-8- yl]ethyl]amino]-6- fluoro-N- methylsulfonyl- pyridine-2- carboxamide | 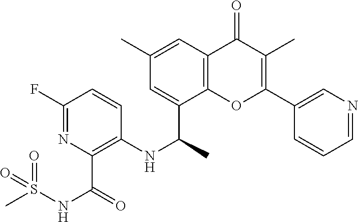 [SMILES: Cc1cc([C@@H](C)Nc2ccc(F)nc2C(=O)NS(C)(=O)=O)c2oc(-c3cccnc3)c(C)c(=O)c2c1] | 511 [M + H]⁺ |
| 57C | 6-Chloro-3-[[(1R)-1- [3,6-dimethyl-2-(1- methylpyrazol-4-yl)-4- oxo-chromen-8- yl]ethyl]amino]pyridine- 2-carboxamide | 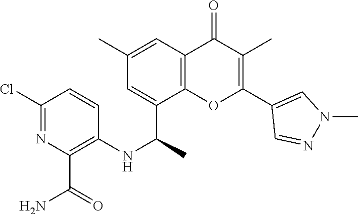 [SMILES: Cc1cc([C@@H](C)Nc2ccc(Cl)nc2C(N)=O)c2oc(-c3cnn(C)c3)c(C)c(=O)c2c1] | 452 [M + H]⁺ |
| 58C | 6-Chloro-3-[(1R)-1- [3,6-dimethyl-2-(1- methylpyrazol-4-yl)-4- oxo-chromen-8- yl]ethyl]amino]-N- methoxy-pyridine-2- carboxamide | 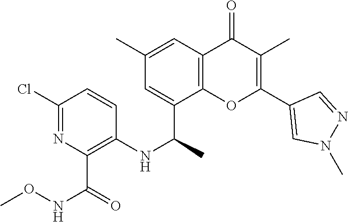 | 482 [M + H]⁺ |
| 59C | 6-Chloro-3-[[(1R)-1- [3-chloro-6-methyl-2- (1-methylpyrazol-4-yl)- 4-oxo-chromen-8- yl]ethyl]amino]-N- methylsulfonyl- pyridine-2- carboxamide | 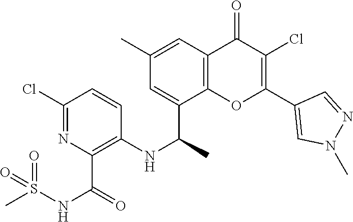 | 550 [M + H]⁺ |
| 60C | 6-Chloro-3-[[(1R)-1- [3,6-dimethyl-2-(1- methylpyrazol-4-yl)-4- oxo-chromen-8- yl]ethyl]amino]-N- methoxy-N-methyl- pyridine-2- carboxamide | 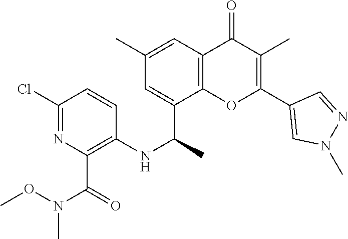 | 496 [M + H]⁺ |
| 61C | 6-Chloro-3-[[(1R)-1- [3,6-dimethyl-2-(1- methylpyrazol-4-yl)-4- oxo-chromen-8- yl]ethyl]amino]-N- hydroxy-N-methyl- pyridine-2- carboxamide | 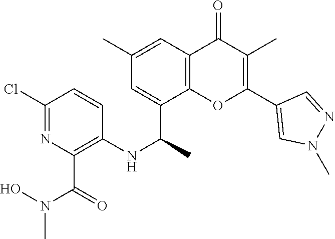 [SMILES: Cc1cc([C@@H](C)Nc2ccc(Cl)nc2C(=O)N(C)O)c2oc(-c3cnn(C)c3)c(C)c(=O)c2c1] | 482 [M + H]⁺ |

¹Ammonium chloride used instead of ammonia and DMF used instead of DMA.
²DCM used as solvent.

### Example 189A: 3-[[(1R)-1-(3,6-Dimethyl-4-oxo-2-phenyl-chromen-8-yl)ethyl]amino]-N-(1,3,4-oxadiazol-2-ylmethyl)pyridine-2-carboxamide

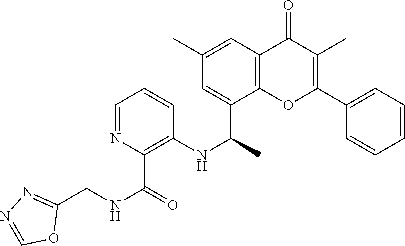 [SMILES: Cc1cc([C@@H](C)Nc2cccnc2C(=O)NCc2nnco2)c2oc(-c3ccccc3)c(C)c(=O)c2c1]

A mixture of 3-[[(1R)-1-(3,6-dimethyl-4-oxo-2-phenyl-chromen-8-yl)ethyl]amino]pyridine-2-carboxylic acid (0.20 g, 0.48 mmol), BOP (0.43 g, 0.97 mmol), triethylamine (0.15 g, 1.45 mmol), and 1,3,4-oxadiazol-2-ylmethanamine hydrochloride (0.1 g, 0.72 mmol) in 6 mL of DMF was stirred overnight at rt under a nitrogen atmosphere. The reaction was purified by reversed phase chromatography eluted with 40% to 50% ACN in water (with 0.1% NH₄OH) followed by preparative HPLC (YMC-Actus Triart C18 ExRS column) eluted with 42% to 51% ACN in water (with 0.1% NH₄OH) to give the title compound (3.6 mg, 1%) as a yellow solid. MS ES+ m/z 496 [M+H]⁺.

### Example 190A: 3-[[(1R)-1-(3,6-dimethyl-4-oxo-2-phenyl-chromen-8-yl)ethyl]amino]-6-methyl-pyridine-2-carboxamide

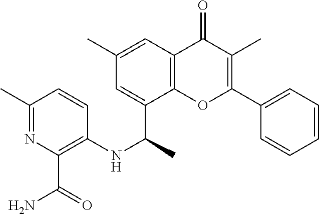 [SMILES: Cc1cc([C@@H](C)Nc2ccc(C)nc2C(N)=O)c2oc(-c3ccccc3)c(C)c(=O)c2c1]

A solution of 3-[[(1R)-1-(3,6-dimethyl-4-oxo-2-phenyl-chromen-8-yl)ethyl]amino]-6-methyl-pyridine-2-carboxylic acid (75 mg, 0.18 mmol) in 3 mL of DMF was treated with HATU (87 mg, 0.23 mmol), ammonium chloride (94 mg, 1.8 mmol), and DIPEA (270 mg, 2.1 mmol) and allowed to stir at rt for 16 h. The reaction mixture was diluted with EtOAc, washed with water, and the organics collected, dried over MgSO₄, filtered, and concentrated to give the title compound (28 mg, 37%). MS ES+ m/z 428 [M+H]⁺.

The following compounds in Table 67 were made in a similar way as described for 3-[[(1R)-1-(3,6-dimethyl-4-oxo-2-phenyl-chromen-8-yl)ethyl]amino]-6-methyl-pyridine-2-carboxamide. Various methods were used to purify the compounds, which would be apparent to one skilled in the art.

TABLE 67

| Example | | | MS ES+ |
|---|---|---|---|
| # | Chemical Name | Structure | m/z |
| 191A | 3-[[(1R)-1-(3,6-Dimethyl- 4-oxo-2-phenyl-chromen- 8-yl)ethyl]amino]-N,6- dimethyl-pyridine-2- carboxamide | 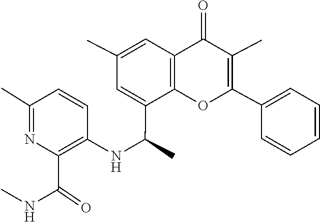 [SMILES: CNC(=O)c1nc(C)ccc1N[C@H](C)c1cc(C)cc2c(=O)c(C)c(-c3ccccc3)oc12] | 442 [M + H]⁺ |
| 192A | 2-[[(1R)-1-[3,6-Dimethyl- 2-(2-methylindazol-5-yl)- 4-oxo-chromen-8-yl]- ethyl]amino]benzamide | 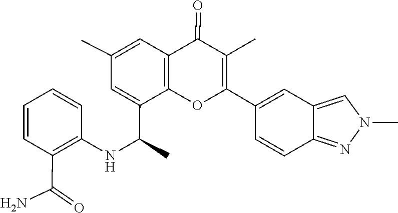 [SMILES: Cc1cc([C@@H](C)Nc2ccccc2C(N)=O)c2oc(-c3ccc4nn(C)cc4c3)c(C)c(=O)c2c1] | 468 [M + H]⁺ |
| 193A | 6-Chloro-3-[(1R)-1-[3,6- dimethyl-4-oxo-2-(3- pyridyl)chromen-8-yl]- ethyl]amino]pyridine-2- carboxamide | 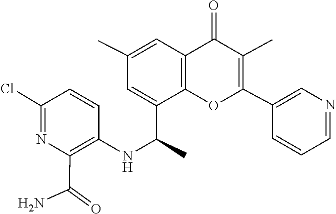 [SMILES: Cc1cc([C@@H](C)Nc2ccc(Cl)nc2C(N)=O)c2oc(-c3cccnc3)c(C)c(=O)c2c1] | 448 [M + H]⁺ |
| 194A | 3-[[(1R)-1-[3,6-Dimethyl- 2-(2-methylindazol-5-yl)- 4-oxo-chromen-8-yl]- ethyl]amino]pyridine-2- carboxamide | 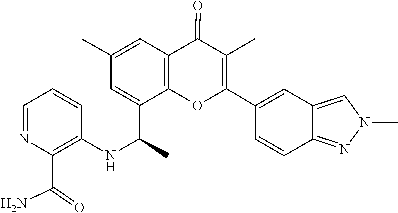 [SMILES: Cc1cc([C@@H](C)Nc2cccnc2C(N)=O)c2oc(-c3ccc4nn(C)cc4c3)c(C)c(=O)c2c1] | 468 [M + H]⁺ |
| 195A | 3-[[(1R)-1-[3,6-Dimethyl- 4-oxo-2-(3-pyridyl)chro- men-8-yl]ethyl]amino]-6- fluoro-pyridine-2-carbox- amide | 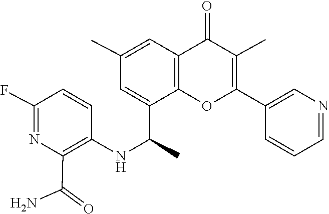 [SMILES: Cc1cc([C@@H](C)Nc2ccc(F)nc2C(N)=O)c2oc(-c3cccnc3)c(C)c(=O)c2c1] | 433 [M + H]⁺ |
| 196A | 6-Chloro-3-[(1R)-1-[6- methyl-2-(2-methyl- indazol-5-yl)-4-oxo- chromen-8-yl]ethyl]- amino]pyridine-2- carboxamide | 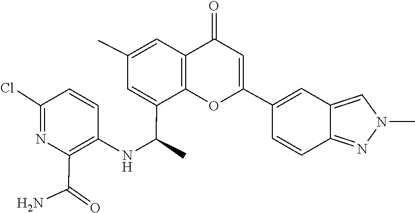 [SMILES: Cc1cc([C@@H](C)Nc2ccc(Cl)nc2C(N)=O)c2oc(-c3ccc4nn(C)cc4c3)cc(=O)c2c1] | 488 [M + H]⁺ |
| 197A | 6-Chloro-3-[[(1R)-1-[3,6- dimethyl-2-(2-methyl- indazol-5-yl)-4-oxo- chromen-8-yl]ethyl]- amino]pyridine-2- carboxamide | 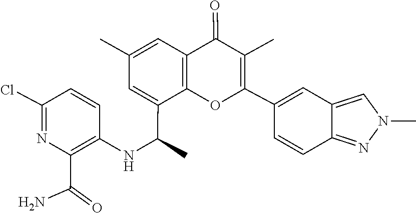 [SMILES: Cc1cc([C@@H](C)Nc2ccc(Cl)nc2C(N)=O)c2oc(-c3ccc4nn(C)cc4c3)c(C)c(=O)c2c1] | 502 [M + H]⁺ |
| 198A | 6-Chloro-3-[[(1R)-1-[3,6- dimethyl-4-oxo-2-(6-oxo- 1H-pyridin-2-yl)chromen- 8-yl]ethyl]amino]pyridine- 2-carboxamide | 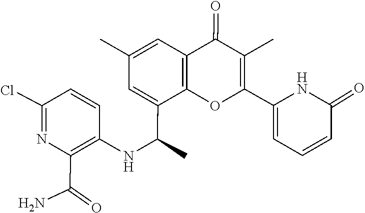 [SMILES: Cc1cc([C@@H](C)Nc2ccc(Cl)nc2C(N)=O)c2oc(-c3cccc(=O)[nH]3)c(C)c(=O)c2c1] | 465 [M + H]⁺ |
| 199A¹ | 2-[[(1R)-1-(6-Methyl-4- oxo-2-phenyl-3-thiazol- 5-yl-chromen-8-yl)ethyl]- amino]benzamide | 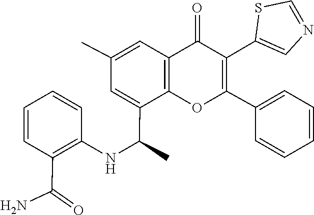 [SMILES: Cc1cc([C@@H](C)Nc2ccccc2C(N)=O)c2oc(-c3ccccc3)c(-c3cncs3)c(=O)c2c1] | 482 [M + H]⁺ |
| 62C | 6-[[(1R)-1-[3,6-Dimethyl- 4-oxo-2-(3-pyridyl)- chromen-8-yl]ethyl]- amino]-2,3-difluoro-N- methoxy-benzamide | 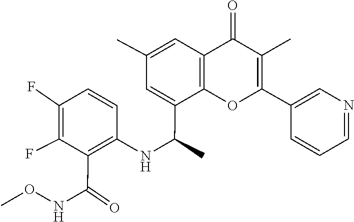 [SMILES: CONC(=O)c1c(N[C@H](C)c2cc(C)cc3c(=O)c(C)c(-c4cccnc4)oc23)ccc(F)c1F] | 480 [M + H]⁺ |
| 63C² | 6-[[(1R)-1-[3,6-Dimethyl- 4-oxo-2-(3-pyridyl)- chromen-8-yl]ethyl]- amino]-2,3-difluoro- benzamide | 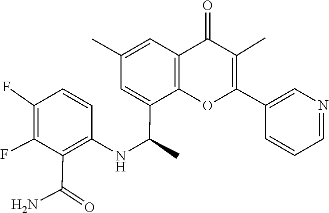 [SMILES: Cc1cc([C@@H](C)Nc2ccc(F)c(F)c2C(N)=O)c2oc(-c3cccnc3)c(C)c(=O)c2c1] | 450 [M + H]⁺ |
| 64C | 6-[(1R)-1-[3,6-Dimethyl- 2-(1-methylpyrazol-4-yl)- 4-oxo-chromen-8-yl]- ethyl]amino]-2,3-difluoro- N-methoxy-benzamide | 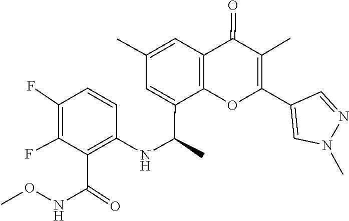 [SMILES: CONC(=O)c1c(N[C@H](C)c2cc(C)cc3c(=O)c(C)c(-c4cnn(C)c4)oc23)ccc(F)c1F] | 483 [M + H]⁺ |
| 65C² | 6-[[(1R)-1-[3,6-Dimethyl- 2-(1-methylpyrazol-4- yl)-4-oxo-chromen-8-yl]- ethyl]amino]-2,3-difluoro- benzamide | 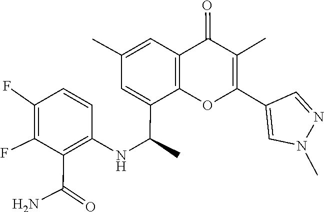 [SMILES: Cc1cc([C@@H](C)Nc2ccc(F)c(F)c2C(N)=O)c2oc(-c3cnn(C)c3)c(C)c(=O)c2c1] | 453 [M + H]⁺ |
| 66C² | 4-[[(1R)-1-[3,6-Dimethyl- 2-(2-methylindazol-5-yl)- 4-oxo-chromen-8-yl]- ethyl]amino]-6-methyl- 2-oxo-1H-pyridine-3- carboxamide | 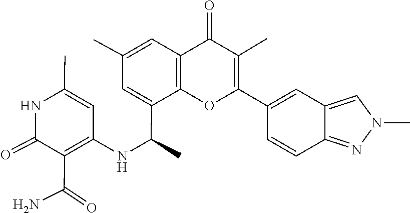 [SMILES: Cc1cc([C@@H](C)Nc2cc(C)[nH]c(=O)c2C(N)=O)c2oc(-c3ccc4nn(C)cc4c3)c(C)c(=O)c2c1] | 498 [M + H]⁺ |
| 67C² | 6-[[(1R)-1-[3,6-Dimethyl- 2-(2-methylindazol-5-yl)- 4-oxo-chromen-8-yl]- ethyl]amino]-2,3-difluoro- benzamide | 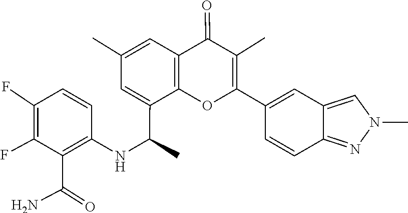 [SMILES: Cc1cc([C@@H](C)Nc2ccc(F)c(F)c2C(N)=O)c2oc(-c3ccc4nn(C)cc4c3)c(C)c(=O)c2c1] | 503 [M + H]⁺ |
| 68C | 2-[[(1R)-1-[3,6-Dimethyl- 2-(2-methylindazol-5-yl)- 4-oxo-chromen-8-yl]- ethyl]amino]-N-methoxy- benzamide | 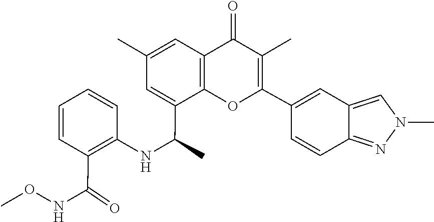 | 497 [M + H]⁺ |
| 69C | 2-[[(1R)-1-[3,6-Dimethyl- 2-(2-methylindazol-5-yl)- 4-oxo-chromen-8-yl]- ethyl]amino]benzene carbohydroxamic acid | 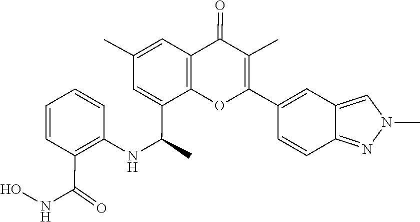 [SMILES: Cc1cc([C@@H](C)Nc2ccccc2C(=O)NO)c2oc(-c3ccc4nn(C)cc4c3)c(C)c(=O)c2c1] | 483 [M + H]⁺ |
| 70C² | 2-[[(1R)-1-[3,6-Dimethyl- 2-(2-methylindazol-5-yl)- 4-oxo-chromen-8-yl]- ethyl]amino]-6-fluoro- benzamide | 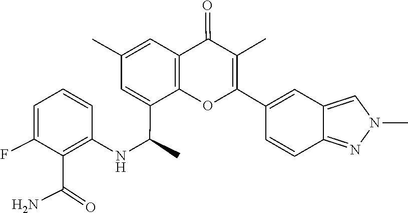 [SMILES: Cc1cc([C@@H](C)Nc2cccc(F)c2C(N)=O)c2oc(-c3ccc4nn(C)cc4c3)c(C)c(=O)c2c1] | 485 [M + H]⁺ |
| 71C | 3-[6-Chloro-3-[[(1R)-1- [2-[1-[1-(2-hydroxy-2- methyl-propanoyl)azetidin- 3-yl]pyrazol-4-yl]-3,6- dimethyl-4-oxo-chromen- 8-yl]ethyl]amino]-2-pyrid- yl]-4H-1,2,4-oxadiazol-5- one | 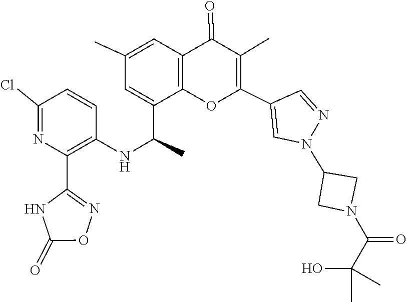 [SMILES: Cc1cc([C@@H](C)Nc2ccc(Cl)nc2-c2noc(=O)[nH]2)c2oc(-c3cnn(C4CN(C(=O)C(C)(C)O)C4)c3)c(C)c(=O)c2c1] | 620 [M + H]⁺ |
| 72C | 6-Chloro-3-[(1R)-1-[3,6- dimethyl-4-oxo-2-[3- (trifluoromethyl)-1- bicyclo[1.1.1]pentanyl] chromen-8-l]ethyl]amino]- pyridine-2-carboxamide | 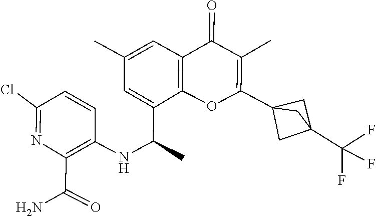 [SMILES: Cc1cc([C@@H](C)Nc2ccc(Cl)nc2C(N)=O)c2oc(C34CC(C(F)(F)F)(C3)C4)c(C)c(=O)c2c1] | 506 [M + H]⁺ |
| 73C | 6-Chloro-3-[1-[6-fluoro-3- methyl-2-(1-methylpyr- azol-4-yl)-4-oxo-chromen- 8-yl]ethylamino]pyridine- 2-carboxamide, Isomer 1 | 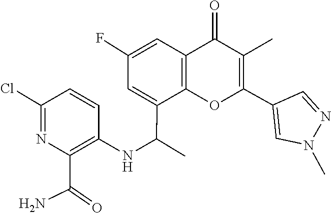 [SMILES: Cc1c(-c2cnn(C)c2)oc2c(C(C)Nc3ccc(Cl)nc3C(N)=O)cc(F)cc2c1=O] | 456 [M + H]⁺ |
| 74C | 6-Chloro-3-[[(1R)-1-(2- cyclobutyl-3,6-dimethyl- 4-oxo-chromen-8-yl)- ethyl]amino]-N-methyl- sulfonyl-pyridine-2- carboxamide | 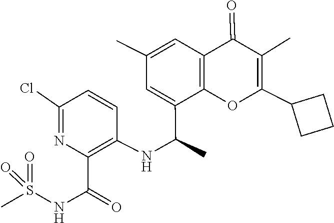 [SMILES: Cc1cc([C@@H](C)Nc2ccc(Cl)nc2C(=O)NS(C)(=O)=O)c2oc(C3CCC3)c(C)c(=O)c2c1] | 504 [M + H]⁺ |
| 75C | 6-Chloro-3-[[(1R)-1-[2- (4,4-difluorocyclohexyl)- 3,6-dimethyl-4-oxo- chromen-8-yl]ethyl]- amino]-N-methylsulfonyl- pyridine-2-carboxamide | 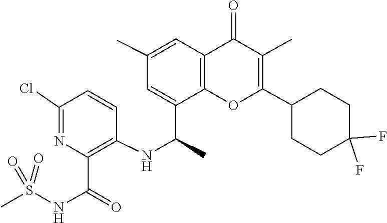 [SMILES: Cc1cc([C@@H](C)Nc2ccc(Cl)nc2C(=O)NS(C)(=O)=O)c2oc(C3CCC(F)(F)CC3)c(C)c(=O)c2c1] | 568 [M + H]⁺ |
| 76C | 6-Chloro-3-[[(1R)-1-[2- (4,4-difluorocyclohexyl)- 3,6-dimethyl-4-oxo- chromen-8-yl]ethyl]- amino]pyridine-2-carbox- amide | 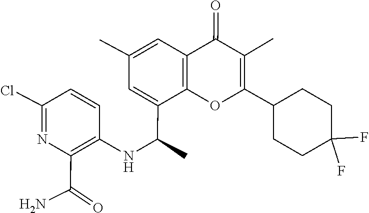 [SMILES: Cc1cc([C@@H](C)Nc2ccc(Cl)nc2C(N)=O)c2oc(C3CCC(F)(F)CC3)c(C)c(=O)c2c1] | 490 [M + H]⁺ |
| 77C² | 4-[[(1R)-1-(3,6-Dimethyl- 4-oxo-2-phenyl-chromen- 8-yl)ethyl]amino]-6-meth- yl-2-oxo-1H-pyridine-3- carboxamide | 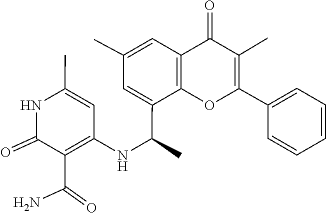 [SMILES: Cc1cc([C@@H](C)Nc2cc(C)[nH]c(=O)c2C(N)=O)c2oc(-c3ccccc3)c(C)c(=O)c2c1] | 444 [M + H]⁺ |
| 78C | 6-Chloro-3-[(1R)-1-(2- cyclopropyl-3,6-dimethyl- 4-oxo-chromen-8-yl)- ethyl]amino]-N-methyl- sulfonyl-pyridine-2- carboxamide | 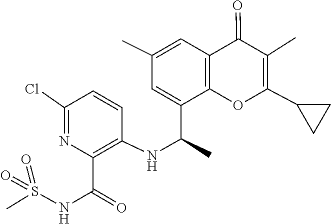 [SMILES: Cc1cc([C@@H](C)Nc2ccc(Cl)nc2C(=O)NS(C)(=O)=O)c2oc(C3CC3)c(C)c(=O)c2c1] | 490 [M + H]⁺ |
| 79C | 6-Chloro-3-[[(1R)-1-(2- cyclopropyl-3,6-dimethyl- 4-oxo-chromen-8-yl)- ethyl]amino]pyridine-2- carboxamide | 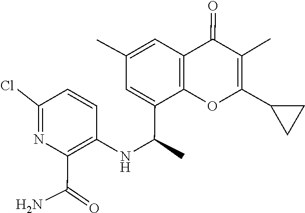 [SMILES: Cc1cc([C@@H](C)Nc2ccc(Cl)nc2C(N)=O)c2oc(C3CC3)c(C)c(=O)c2c1] | 412 [M + H]⁺ |

¹Ammonia (0.5M in 1,4-dioxane) used instead of ammonium chloride.
²Ammonia in 1,4-dioxane used in this reaction.

### Example 200A: 6-Chloro-3-[[(1R)-1-[2-(2,6-difluorophenyl)-3,6-dimethyl-4-oxo-chromen-8-yl]ethyl]amino]pyridine-2-carboxamide

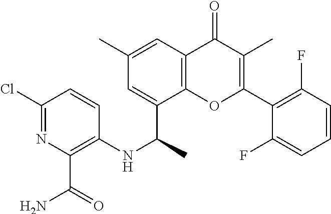 [SMILES: Cc1cc([C@@H](C)Nc2ccc(Cl)nc2C(N)=O)c2oc(-c3c(F)cccc3F)c(C)c(=O)c2c1]

Combined 6-chloro-3-[[(1R)-1-[2-(2,6-difluorophenyl)-3,6-dimethyl-4-oxo-chromen-8-yl]ethyl]amino]pyridine-2-carbonitrile (60 mg, 0.13 mmol) and dihydrogen tris(dimethylphosphinito)hydroplatinate(2-) (Ghaffar-Parkins catalyst, 28 mg, 0.06 mmol) in EtOH (0.5 mL) and water (0.5 mL), degassed for 10 min, capped, and stirred at 80° C. overnight. The reaction was cooled to room temperature and diluted with 15 mL of DCM and water. Extracted with DCM (3×10 mL). The organics were combined, passed through a phase separator frit and concentrated. The residue was purified by reversed phase chromatography eluted with ACN (with 0.1% formic acid) in water (with 0.1% formic acid) to give the title compound (40 mg, 64%). MS ES+ m/z 484 [M+H]⁺.

The following compound in Table 68 was made in a similar way as described for 6-chloro-3-[[(1R)-1-[2-(2,6-difluorophenyl)-3,6-dimethyl-4-oxo-chromen-8-yl]ethyl]amino]pyridine-2-carboxamide. Various methods were used to purify the compound, which would be apparent to one skilled in the art.

TABLE 68

| Example | | | MS ES+ |
|---|---|---|---|
| # | Chemical Name | Structure | m/z |
| 80C | 6-Chloro-3-[(1R)-1- [3,6-dimethyl-2-(1- methylindazol-3-yl)- 4-oxo-chromen-8- yl]ethyl]amino]- pyridine-2-carbox- amide | 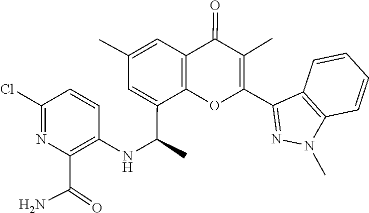 [SMILES: Cc1cc([C@@H](C)Nc2ccc(Cl)nc2C(N)=O)c2oc(-c3nn(C)c4ccccc34)c(C)c(=O)c2c1] | 502 [M + H]⁺ |

### Example 81C: 3,6-Dimethyl-2-phenyl-8-[(1R)-1-[2-(4H-1,2,4-triazol-3-yl)anilino]ethyl]chromen-4-one

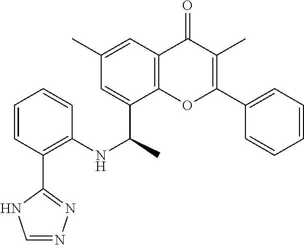 [SMILES: Cc1cc([C@@H](C)Nc2ccccc2-c2nnc[nH]2)c2oc(-c3ccccc3)c(C)c(=O)c2c1]

2-[[(1R)-1-(3,6-Dimethyl-4-oxo-2-phenyl-chromen-8-yl)ethyl]amino]benzamide (300 mg, 0.73 mmol) was dissolved in 1,1-dimethoxy-N,N-dimethylmethanamine (3 g, 20 mmol) and stirred at 120° C. for 2 h. The 1,1-dimethoxy-N,N-dimethylmethanamine was removed under reduced pressure and the residue redissolved in acetic acid (3 mL). The reaction was treated with hydrazine hydrate (1.82 g, 36.4 mmol) and then stirred at 90° C. for 20 h. Added EtOAc (10 mL) and then concentrated the reaction under reduced pressure. The resulting solid was dissolved in EtOAc (20 mL), washed with water (2×20 mL) and brine (20 mL). The organic layer was concentrated under reduced pressure and the residue purified by reversed phase chromatography on a CSH column eluted with 20% to 80% ACN in 10 mM NH₄HCO₃ (with 5% MeOH) to give the title compound (22.7 mg, 7%) as an off-white solid. ES/MS m/z 437 (M+H).

### Example 57A: 8-[(1R)-1-[2-(5-Amino-1,3,4-oxadiazol-2-yl)anilino]ethyl]-3,6-dimethyl-2-phenyl-chromen-4-one

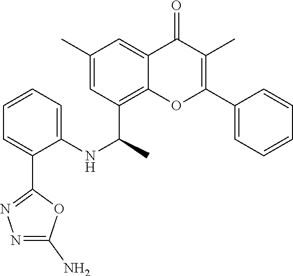 [SMILES: Cc1cc([C@@H](C)Nc2ccccc2-c2nnc(N)o2)c2oc(-c3ccccc3)c(C)c(=O)c2c1]

To a solution of 2-[[(1R)-1-(3,6-dimethyl-4-oxo-2-phenyl-chromen-8-yl)ethyl]amino]benzohydrazide (0.100 g, 0.234 mmol) in EtOH (2 mL) was added K2CO₃ (0.065 g, 0.47 mmol) and cyanogen bromide (0.074 g, 0.70 mmol). The reaction was sealed and heated at 70° C. for 17 h. The mixture was diluted with EtOAc and washed with water, then the aqueous layer was extracted twice with EtOAc. The combined organic extracts were dried over Na₂SO₄, filtered, and concentrated. The crude material was purified by silica gel chromatography eluted with a gradient of 0% to 100% EtOAc in hexanes, then re-purified by reverse phase chromatography eluted with a gradient of 0% to 100% ACN in aqueous formic acid (0.1%). Fractions containing the product were combined, washed with saturated aqueous sodium chloride, and extracted with IPA:CHCl₃ (1:3). The combined extracts were dried over MgSO₄, filtered, and concentrated to give the title compound (0.011 g, 10%) as a white solid. MS ES+ m/z 453 [M+H]⁺.

The following compounds in Table 69 were made in a similar way as described for 8-[(1R)-1-[2-(5-amino-1,3,4-oxadiazol-2-yl)anilino]ethyl]-3,6-dimethyl-2-phenyl-chromen-4-one. Various methods were used to purify the compounds, which would be apparent to one skilled in the art.

TABLE 69

| Example | | | MS ES+ |
|---|---|---|---|
| # | Chemical Name | Structure | m/z |
| 58A | 8-[(1R)-1-[[2-(5-Amino- 1,3,4-oxadiazol-2-yl)- 3-pyridyl]amino]ethyl]- 3,6-dimethyl-2-phenyl- chromen-4-one | 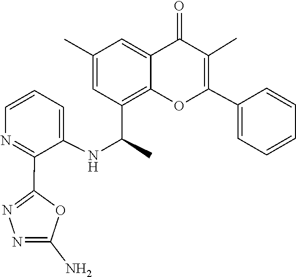 [SMILES: Cc1cc([C@@H](C)Nc2cccnc2-c2nnc(N)o2)c2oc(-c3ccccc3)c(C)c(=O)c2c1] | 454 [M + H]⁺ |
| 201A | 8-[(1R)-1-[2-(5-Amino- 1,3,4-oxadiazol-2-yl)-6- chloro-3-pyridyl]amino]- ethyl]-3,6-dimethyl-2- phenyl-chromen-4-one | 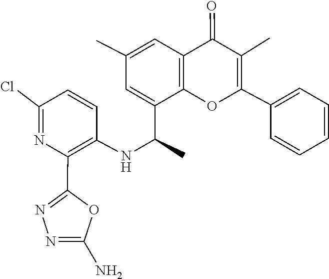 [SMILES: Cc1cc([C@@H](C)Nc2ccc(Cl)nc2-c2nnc(N)o2)c2oc(-c3ccccc3)c(C)c(=O)c2c1] | 488 [M + H]⁺ |
| 202A | 8-[(1R)-1-[2-(5-Amino- 1,3,4-oxadiazol-2-yl)-6- methyl-3-pyridyl]amino]- ethyl]-3,6-dimethyl-2- (2-methylindazol-5-yl)- chromen-4-one | 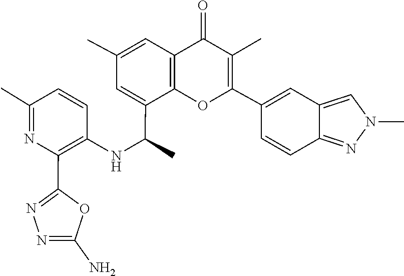 | 522 [M + H]⁺ |
| 203A | 8-[(1R)-1-[[2-(5-Amino- 1,3,4-oxadiazol-2-yl)-6- methyl-3-pyridyl]amino]- ethyl]-3,6-dimethyl-2- phenyl-chromen-4-one | 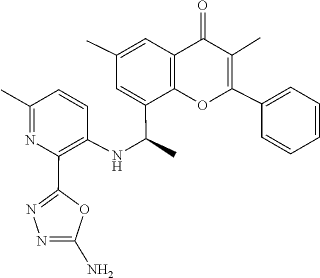 [SMILES: Cc1cc([C@@H](C)Nc2ccc(C)nc2-c2nnc(N)o2)c2oc(-c3ccccc3)c(C)c(=O)c2c1] | 468 [M + H]⁺ |
| 204A | 8-[(1R)-1-[2-(5-Amino- 1,3,4-oxadiazol-2-yl)- 3,4-difluoro-anilino]- ethyl]-3,6-dimethyl-2- phenyl-chromen-4-one | 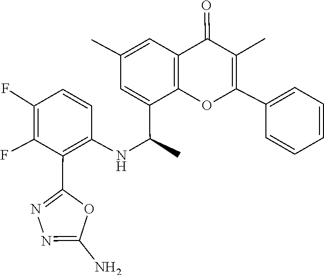 [SMILES: Cc1cc([C@@H](C)Nc2ccc(F)c(F)c2-c2nnc(N)o2)c2oc(-c3ccccc3)c(C)c(=O)c2c1] | 489 [M + H]⁺ |
| 205A | 8-[(1R)-1-[2-(5-Amino- 1,3,4-oxadiazol-2-yl)-6- methyl-3-pyridyl]amino]- ethyl]-2-(3,4-difluoro- phenyl)-3,6-dimethyl- chromen-4-one | 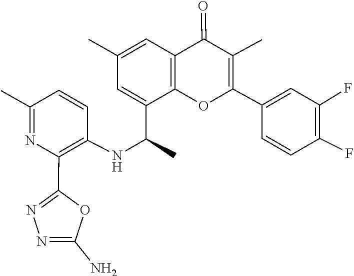 [SMILES: Cc1cc([C@@H](C)Nc2ccc(C)nc2-c2nnc(N)o2)c2oc(-c3ccc(F)c(F)c3)c(C)c(=O)c2c1] | 504 [M + H]⁺ |
| 206A | 8-[(1R)-1-[2-(5-Amino- 1,3,4-oxadiazol-2-yl)-3- fluoro-anilino]ethyl]- 3,6-dimethyl-2-phenyl- chromen-4-one | 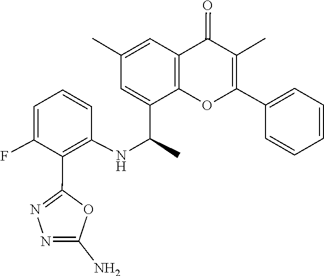 [SMILES: Cc1cc([C@@H](C)Nc2cccc(F)c2-c2nnc(N)o2)c2oc(-c3ccccc3)c(C)c(=O)c2c1] | 471 [M + H]⁺ |
| 207A | 8-[(1R)-1-[[2-(5-Amino- 1,3,4-oxadiazol-2-yl)-3- pyridyl]amino]ethyl]-2- [6-(difluoromethyl)-2- pyridyl]-3,6-dimethyl- chromen-4-one | 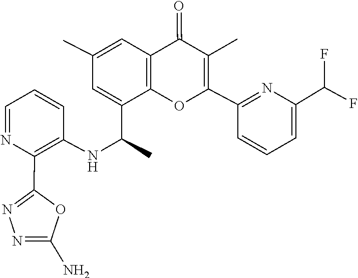 [SMILES: Cc1cc([C@@H](C)Nc2cccnc2-c2nnc(N)o2)c2oc(-c3cccc(C(F)F)n3)c(C)c(=O)c2c1] | 505 [M + H]⁺ |

### Example 59A: 3,6-Dimethyl-8-[(1R)-1-[2-(1,3,4-oxadiazol-2-yl)anilino]ethyl]-2-phenyl-chromen-4-one

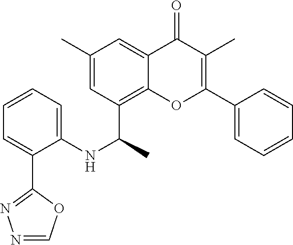 [SMILES: Cc1cc([C@@H](C)Nc2ccccc2-c2nnco2)c2oc(-c3ccccc3)c(C)c(=O)c2c1]

To a solution of 2-[[(1R)-1-(3,6-dimethyl-4-oxo-2-phenyl-chromen-8-yl)ethyl]amino]benzohydrazide (0.100 g, 0.234 mmol) in triethyl orthoformate (2 mL, 10 mmol) was added TFA (0.005 mL). The reaction was sealed and heated in a microwave reactor at 120° C. for 1 h. The mixture was diluted with EtOAc and washed with saturated aqueous NaHCO₃ and saturated aqueous sodium chloride. The organic layer was dried over Na₂SO₄, filtered, and concentrated. The residue was recrystallized in MTBE/hexanes to obtain the title compound (0.038 g, 37%) as a tan solid. MS ES+ m/z 438 [M+H]⁺.

The following compounds in Table 70 were made in a similar way as described for 3,6-dimethyl-8-[(1R)-1-[2-(1,3,4-oxadiazol-2-yl)anilino]ethyl]-2-phenyl-chromen-4-one. Various methods were used to purify the compounds, which would be apparent to one skilled in the art.

TABLE 70

| Example | | | MS ES+ |
|---|---|---|---|
| # | Chemical Name | Structure | m/z |
| 60A | 3,6-Dimethyl-8-[(1R)-1- [[2-(1,3,4-oxadiazol-2-yl)- 3-pyridyl]amino]ethyl]-2- phenyl-chromen-4-one | 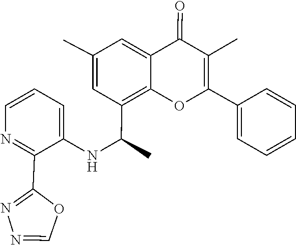 [SMILES: Cc1cc([C@@H](C)Nc2cccnc2-c2nnco2)c2oc(-c3ccccc3)c(C)c(=O)c2c1] | 439 [M + H]⁺ |
| 208A | 8-[(1R)-1-[[6-Chloro-2- (1,3,4-oxadiazol-2-yl)-3- pyridyl]amino]ethyl]-3,6- dimethyl-2-phenyl- chromen-4-one | 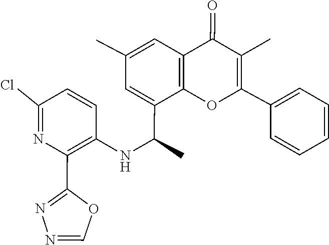 [SMILES: Cc1cc([C@@H](C)Nc2ccc(Cl)nc2-c2nnco2)c2oc(-c3ccccc3)c(C)c(=O)c2c1] | 473 [M + H]⁺ |

### Example 61A: 5-[2-[[(1R)-1-(3,6-Dimethyl-4-oxo-2-phenyl-chromen-8-yl)ethyl]amino]phenyl]-3H-1,3,4-oxadiazol-2-one

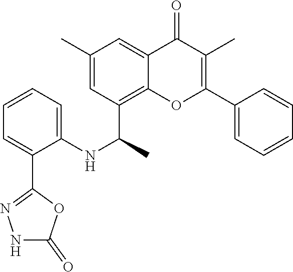 [SMILES: Cc1cc([C@@H](C)Nc2ccccc2-c2n[nH]c(=O)o2)c2oc(-c3ccccc3)c(C)c(=O)c2c1]

To a solution of 2-[[(1R)-1-(3,6-dimethyl-4-oxo-2-phenyl-chromen-8-yl)ethyl]amino]benzohydrazide (0.080 g, 0.19 mmol) in DCM (2 mL) was added DIPEA (0.065 mL, 0.37 mmol). In a separate vessel, triphosgene (0.028 g, 0.094 mmol) was dissolved in DCM (2 mL), sonicating for complete dissolution of the reagent before use. The triphosgene/DCM solution was added and the reaction was stirred at rt for 1 h. The mixture was concentrated and purified by silica gel chromatography eluted with a gradient of 0% to 100% EtOAc in hexanes. Fractions with the desired product were combined and concentrated, and the residue was recrystallized from DCM/hexanes to give the title compound (0.036 g, 42%) as a pale tan solid. MS ES+ m/z 454 [M+H]⁺.

### Example 62A: 8-[(1R)-1-[2-(5-Amino-4H-1,2,4-triazol-3-yl)anilino]ethyl]-3,6-dimethyl-2-phenyl-chromen-4-one

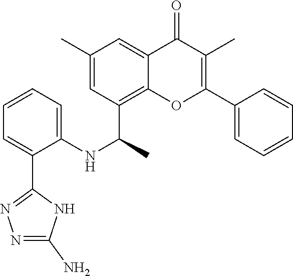 [SMILES: Cc1cc([C@@H](C)Nc2ccccc2-c2nnc(N)[nH]2)c2oc(-c3ccccc3)c(C)c(=O)c2c1]

To a solution of 2-[[(1R)-1-(3,6-dimethyl-4-oxo-2-phenyl-chromen-8-yl)ethyl]amino]benzohydrazide (0.100 g, 0.234 mmol) in ethanol (2 mL) was added 2-methylisothiourea; sulfuric acid (0.076 mL, 0.35 mmol). The reaction was sealed and heated at 95° C. for 17 h. Added NaOH (0.028 g, 0.70 mmol), sealed, and heated at 95° C. for 2 h. Added NaOH (0.056 g, 1.4 mmol), sealed, and heated at 105° C. overnight. The mixture was diluted with DCM and saturated aqueous NH₄Cl. Extracted with IPA:CHCl₃ (1:3). The organic extracts were dried over MgSO₄, filtered, concentrated, and the residue was recrystallized from DCM/hexanes to give the title compound (0.020 g, 19%) as a white solid. MS ES+ m/z 452 [M+H]⁺.

### Example 63A: 3-[3-[[(1R)-1-(3,6-Dimethyl-4-oxo-2-phenyl-chromen-8-yl)ethyl]amino]-2-pyridyl]-1,4-dihydro-1,2,4-triazol-5-one

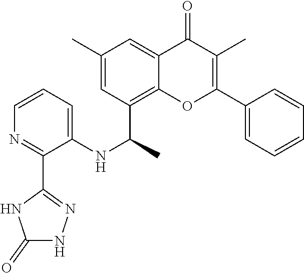 [SMILES: Cc1cc([C@@H](C)Nc2cccnc2-c2n[nH]c(=O)[nH]2)c2oc(-c3ccccc3)c(C)c(=O)c2c1]

3-[[(1R)-1-(3,6-Dimethyl-4-oxo-2-phenyl-chromen-8-yl)ethyl]amino]pyridine-2-carbonitrile (0.100 g, 0.253 mmol) and carbazic acid ethylester (0.080 g, 0.77 mmol) were combined in NMP (2 mL). The reaction was sealed and heated in a microwave reactor at 160° C. for 2 h. Added carbazic acid ethyl ester (0.080 g, 0.77 mmol). The reaction was sealed and heated at 160° C. overnight. The mixture was concentrated, and the residue was purified by reverse phase chromatography eluted with a gradient of 0% to 100% ACN in aqueous formic acid (0.1%). Fractions containing the desired product were combined, washed with saturated aqueous sodium chloride, and extracted with IPA:CHCl₃ (1:3). The organic extracts were dried over MgSO₄, filtered, concentrated, and the residue was recrystallized from DCM/hexanes to give the title compound (0.052 g, 46%) as a white solid. MS ES+ m/z 454 [M+H]⁺

### Example 64A: 3,6-Dimethyl-8-[(1R)-1-[2-(3-methyl-1,2,4-oxadiazol-5-yl)anilino]ethyl]-2-phenyl-chromen-4-one

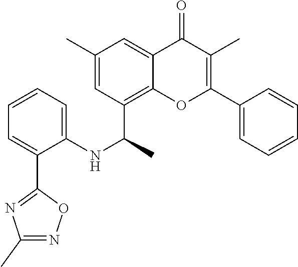 [SMILES: Cc1cc([C@@H](C)Nc2ccccc2-c2nc(C)no2)c2oc(-c3ccccc3)c(C)c(=O)c2c1]

To a solution of 2-[[(1R)-1-(3,6-dimethyl-4-oxo-2-phenyl-chromen-8-yl)ethyl]amino]benzoic acid (0.20 g, 0.48 mmol) in DMF (4 mL) was added (Z)—N′-hydroxyacetimidamide (0.036 g, 0.48 mmol), propylphosphonic anhydride (50 wt % in EtOAc, 0.29 mL, 0.48 mmol) and DIPEA (0.084 mL, 0.48 mmol). The reaction was stirred at rt for 2 h, then charged with additional (Z)—N′-hydroxyacetimidamide (0.054 g, 0.73 mmol), propylphosphonic anhydride (50 wt % in EtOAc, 0.43 mL, 0.73 mmol) and DIPEA (0.13 mL, 0.73 mmol). The reaction was stirred at rt for 2 h, then heated to 110° C. overnight. The mixture was diluted with EtOAc, neutralized with 1N aqueous HCl, and extracted 3× with EtOAc. The combined organic extracts were washed with saturated aqueous NaHCO₃, saturated aqueous sodium chloride, and 10% aqueous LiCl. The organic layer was dried over Na₂SO₄, filtered, and concentrated. The residue was purified by silica gel chromatography eluted with a gradient of 0% to 100% EtOAc in heptane to give the title compound (0.10 g, 46%) as a white solid. MS ES+ m/z 452 [M+H]⁺.

### Example 65A: 3,6-Dimethyl-8-[(1R)-1-(2-oxazol-2-ylanilino)ethyl]-2-phenyl-chromen-4-one

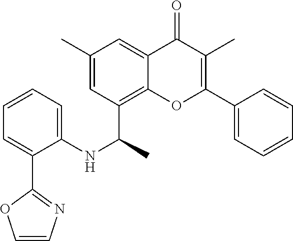 [SMILES: Cc1cc([C@@H](C)Nc2ccccc2-c2ncco2)c2oc(-c3ccccc3)c(C)c(=O)c2c1]

3,6-Dimethyl-2-phenyl-8-[(1R)-1-[2-(4,4,5,5-tetramethyl-1,3,2-dioxaborolan-2-yl)anilino]ethyl]chromen-4-one (0.050 g, 0.10 mmol), 2-bromooxazole (0.018 g, 0.12 mmol), 1,1′-bis(diphenylphosphino)ferrocenedichloro palladium(II) dichloromethane complex (0.0074 g, 0.010 mmol), and K2CO₃ (1M aqueous, 0.30 mL, 0.30 mmol) were combined in 1,4-dioxane (2 mL) and water (0.5 mL), degassed using argon for 1 min, and stirred at 95° C. for 16 h. The mixture was loaded directly onto a diatomaceous earth plug and purified by reverse phase chromatography eluted with a gradient of 0% to 100% ACN in aqueous NH₄HCO₃ (10 mM, plus 50 MeCH). Fractions containing desired product were concentrated, then dissolved in ACN/water and lyophilized to give the title compound (0.0047 g, 110%). MS ES+ m/z 437 [M+H]⁺.

The following compounds in Table 71 were made in a similar way as described for 3,6-dimethyl-2-phenyl-8-[(1R)-1-(2-pyridazin-3-ylanilino)ethyl]chromen-4-one. Various methods were used to purify the compounds, which would be apparent to one skilled in the art.

TABLE 71

| Example | | | MS ES+ |
|---|---|---|---|
| # | Chemical Name | Structure | m/z |
| 66A | 3,6-Dimethyl-8-[(1R)-1- [2-(4-methyl-1,2,4-triazol- 3-yl)anilino]ethyl]-2- phenyl-chromen-4-one | 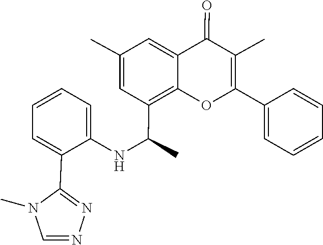 [SMILES: Cc1cc([C@@H](C)Nc2ccccc2-c2nncn2C)c2oc(-c3ccccc3)c(C)c(=O)c2c1] | 451 [M + H]⁺ |
| 67A | 3,6-Dimethyl-2-phenyl-8- [(1R)-1-(2-pyridazin-3- yl)anilino)ethyl]chromen- 4-one | 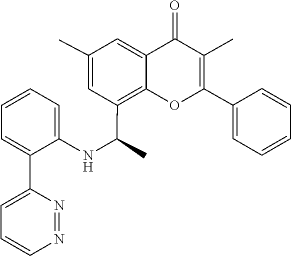 [SMILES: Cc1cc([C@@H](C)Nc2ccccc2-c2cccnn2)c2oc(-c3ccccc3)c(C)c(=O)c2c1] | 448 [M + H]⁺ |
| 68A | 3,6-Dimethyl-8-[(1R)-1- [2-(3-methylimidazol-4- yl)anilino]ethyl]-2- phenyl-chromen-4-one | 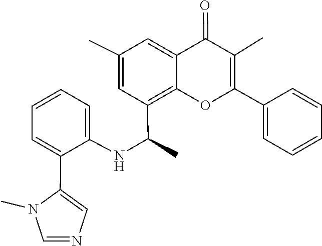 [SMILES: Cc1cc([C@@H](C)Nc2ccccc2-c2cncn2C)c2oc(-c3ccccc3)c(C)c(=O)c2c1] | 450 [M + H]⁺ |
| 69A | 3,6-Dimethyl-8-[(1R)-1- [2-(1-methylimidazol-4- yl)anilino]ethyl]-2- phenyl-chromen-4-one | 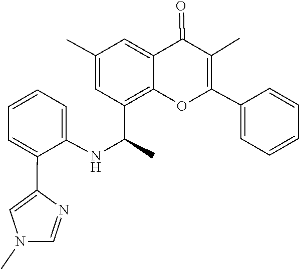 [SMILES: Cc1cc([C@@H](C)Nc2ccccc2-c2cn(C)cn2)c2oc(-c3ccccc3)c(C)c(=O)c2c1] | 450 [M + H]⁺ |
| 70A | 3,6-Dimethyl-2-phenyl-8- [(1R)-1-[2-(1H-triazol-4- yl)anilino]ethyl]chromen- 4-one | 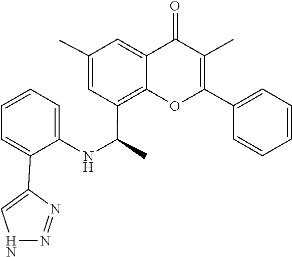 [SMILES: Cc1cc([C@@H](C)Nc2ccccc2-c2c[nH]nn2)c2oc(-c3ccccc3)c(C)c(=O)c2c1] | 437 [M + H]⁺ |
| 71A | 3,6-Dimethyl-2-(3-pyrid- yl)-8-[(1R)-1-(2-thiazol-4- ylanilino)ethyl]chromen- 4-one | 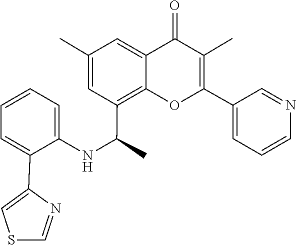 [SMILES: Cc1cc([C@@H](C)Nc2ccccc2-c2cscn2)c2oc(-c3cccnc3)c(C)c(=O)c2c1] | 454 [M + H]⁺ |
| 72A | 3,6-Dimethyl-2-(3- pyridyl)-8-[(1R)-1-(2- thiazol-5-ylanilino)- ethyl]chromen-4-one | 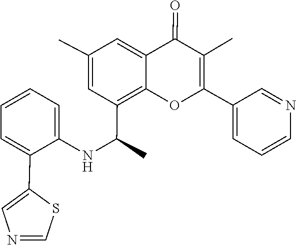 [SMILES: Cc1cc([C@@H](C)Nc2ccccc2-c2cncs2)c2oc(-c3cccnc3)c(C)c(=O)c2c1] | 454 [M + H]⁺ |
| 73A | 3,6-Dimethyl-2-(3-pyrid- yl)-8-[(1R)-1-(2-thiazol-2- ylanilino)ethyl]chromen- 4-one | 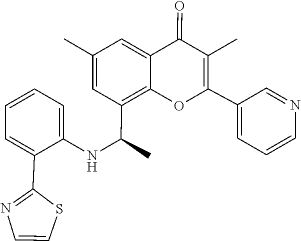 [SMILES: Cc1cc([C@@H](C)Nc2ccccc2-c2nccs2)c2oc(-c3cccnc3)c(C)c(=O)c2c1] | 454 [M + H]⁺ |
| 74A | 8-[(1R)-1-(2-Isothiazol- 5-ylanilino)ethyl]-3,6- dimethyl-2-(3-pyridyl)- chromen-4-one | 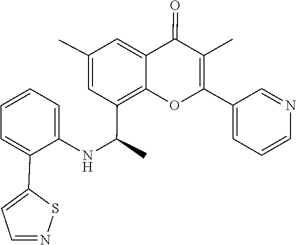 [SMILES: Cc1cc([C@@H](C)Nc2ccccc2-c2ccns2)c2oc(-c3cccnc3)c(C)c(=O)c2c1] | 454 [M + H]⁺ |
| 75A | 8-[(1R)-1-(2-Isothiazol- 3-ylanilino)ethyl]-3,6- dimethyl-2-(3-pyridyl)- chromen-4-one | 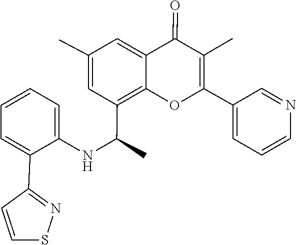 [SMILES: Cc1cc([C@@H](C)Nc2ccccc2-c2ccsn2)c2oc(-c3cccnc3)c(C)c(=O)c2c1] | 454 [M + H]⁺ |
| 209A | 8-[(1R)-1-[(2-Isothiazol- 4-yl-3-pyridyl)amino]- ethyl]-3,6-dimethyl-2-(3- pyridyl)chromen-4-one | 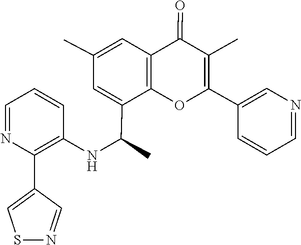 [SMILES: Cc1cc([C@@H](C)Nc2cccnc2-c2cnsc2)c2oc(-c3cccnc3)c(C)c(=O)c2c1] | 455 [M + H]⁺ |
| 210A | 3,6-Dimethyl-2-phenyl-8- [(1R)-1-[2-(4-pyridyl)- anilino]ethyl]chromen-4- one | 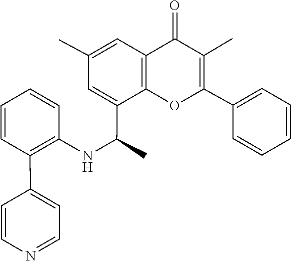 [SMILES: Cc1cc([C@@H](C)Nc2ccccc2-c2ccncc2)c2oc(-c3ccccc3)c(C)c(=O)c2c1] | 447 [M + H]⁺ |
| 211A¹ | 3,6-Dimethyl-2-phenyl- 8-[(1R)-1-(2-pyridazin-4- ylanilino)ethyl]chromen- 4-one | 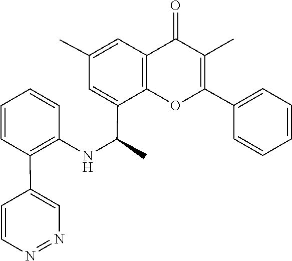 [SMILES: Cc1cc([C@@H](C)Nc2ccccc2-c2ccnnc2)c2oc(-c3ccccc3)c(C)c(=O)c2c1] | 448 [M + H]⁺ |
| 212A | 8-[(1R)-1-[(2-Isothiazol- 4-yl-3-pyridyl)amino]- ethyl]-3,6-dimethyl-2- phenyl-chromen-4-one | 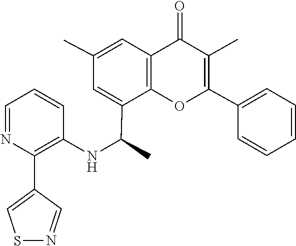 [SMILES: Cc1cc([C@@H](C)Nc2cccnc2-c2cnsc2)c2oc(-c3ccccc3)c(C)c(=O)c2c1] | 454 [M + H]⁺ |
| 213A | 8-[(1R)-1-(2-Isothiazol- 4-ylanilino)ethyl]-3,6- dimethyl-2-(3-pyridyl)- chromen-4-one | 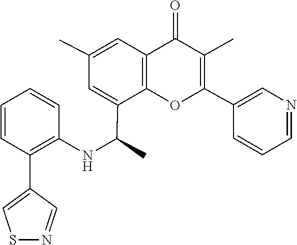 [SMILES: Cc1cc([C@@H](C)Nc2ccccc2-c2cnsc2)c2oc(-c3cccnc3)c(C)c(=O)c2c1] | 454 [M + H]⁺ |
| 214A | 8-[(1R)-1-(2-Isothiazol- 4-ylanilino)ethyl]-3,6- dimethyl-2-phenyl- chromen-4-one | 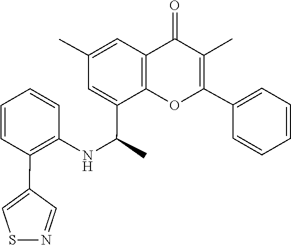 [SMILES: Cc1cc([C@@H](C)Nc2ccccc2-c2cnsc2)c2oc(-c3ccccc3)c(C)c(=O)c2c1] | 453 [M + H]⁺ |
| 215A² | 8-[(1R)-1-[2-(2-Methoxy- pyrimidin-5-yl)anilino]- ethyl]-3,6-dimethyl-2- phenyl-chromen-4-one | 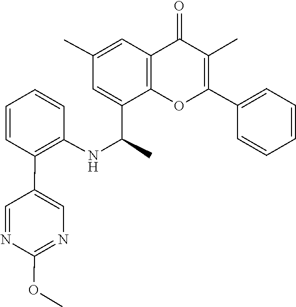 [SMILES: COc1ncc(-c2ccccc2N[C@H](C)c2cc(C)cc3c(=O)c(C)c(-c4ccccc4)oc23)cn1] | 478 [M + H]⁺ |
| 216A² | 6-Chloro-3-[[(1R)-1-(3- isoxazol-4-yl-6-methyl- 4-oxo-2-phenyl-chromen- 8-yl)ethyl]amino]-N- methylsulfonyl-pyridine- 2-carboxamide | 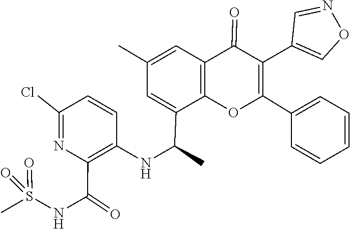 [SMILES: Cc1cc([C@@H](C)Nc2ccc(Cl)nc2C(=O)NS(C)(=O)=O)c2oc(-c3ccccc3)c(-c3cnoc3)c(=O)c2c1] | 579 [M + H]⁺ |

¹ACN used as the solvent.
²2M aqueous Na₂CO₃ used as the base.

### Example 82C: 3,6-Dimethyl-8-[(1R)-1-[[2-(1-methylpyrazol-4-yl)-3-pyridyl]amino]ethyl]-2-(3-pyridyl)chromen-4-one

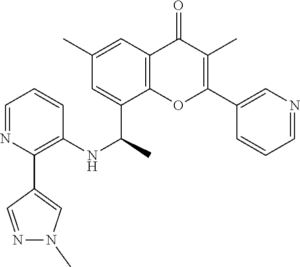 [SMILES: Cc1cc([C@@H](C)Nc2cccnc2-c2cnn(C)c2)c2oc(-c3cccnc3)c(C)c(=O)c2c1]

Combined 8-[(1R)-1-[(2-chloro-3-pyridyl)amino]ethyl]-3,6-dimethyl-2-(3-pyridyl)chromen-4-one (100 mg, 0.25 mmol), (1-methylpyrazol-4-yl)boronic acid (46.5 mg, 0.37 mmol), potassium carbonate (68.1 mg, 0.49 mmol), and tetrakis(triphenylphosphine)palladium(O) (42.7 mg, 0.04 mmol) in 1,4-dioxane (2 mL) and water (0.5 mL). Bubbled argon gas through the reaction for 5 min and then allowed to stir at 60° C. for 16 h. The reaction was filtered, the filtrate concentrated under reduced pressure, and the residue purified by silica gel chromatography eluted with 0% to 100% EtOAc in heptane. The fractions containing product were concentrated under reduced pressure and the residue purified by reversed phase chromatography on C18 eluted with 0% to 100% ACN in 10 mM aqueous NH₄HCO₃ (with 5% MeOH) to give the title compound (58 mg, 52%). ES/MS m/z 452 (M+H).

The following compounds in Table 72 were made in a similar way as described for 3,6-dimethyl-8-[(1R)-1-[[2-(1-methylpyrazol-4-yl)-3-pyridyl]amino]ethyl]-2-(3-pyridyl)chromen-4-one. Various methods were used to purify the compounds, which would be apparent to one skilled in the art.

TABLE 72

| Example | | | MS ES+ |
|---|---|---|---|
| # | Chemical Name | Structure | m/z |
| 83C | 3,6-Dimethyl-8-[(1R)-1- [[2-(1H-pyrazol-3-yl)-3- pyridyl]amino]ethyl]-2- (3-pyridyl)chromen-4- one | 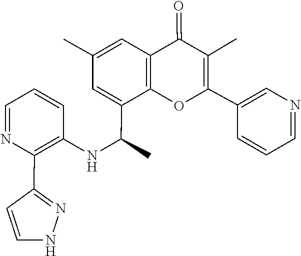 [SMILES: Cc1cc([C@@H](C)Nc2cccnc2-c2cc[nH]n2)c2oc(-c3cccnc3)c(C)c(=O)c2c1] | 438 [M + H]⁺ |
| 84C¹ | 8-[(1R)-1-[(2-Isoxazol- 4-yl-3-pyridyl)amino]- ethyl]-3,6-dimethyl-2-(3- pyridyl)chromen-4-one | 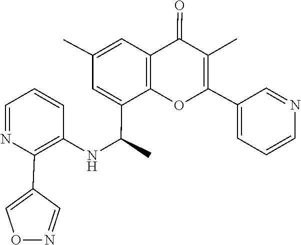 [SMILES: Cc1cc([C@@H](C)Nc2cccnc2-c2cnoc2)c2oc(-c3cccnc3)c(C)c(=O)c2c1] | 439 [M + H]⁺ |
| 85C¹ | 3,6-Dimethyl-8-[(1R)- 1-[(2-oxazol-4-yl-3- pyridyl)amino]ethyl]-2- (3-pyridyl)chromen-4- one | 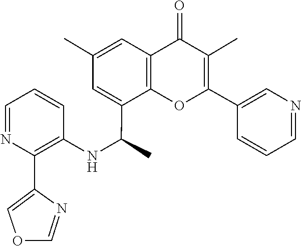 [SMILES: Cc1cc([C@@H](C)Nc2cccnc2-c2cocn2)c2oc(-c3cccnc3)c(C)c(=O)c2c1] | 439 [M + H]⁺ |
| 86C | 3,6-Dimethyl-8-[(1R)- 1-[[2-(3-methylisoxazol- 5-yl)-3-pyridyl]amino]- ethyl]-2-(3-pyridyl)chro- men-4-one | 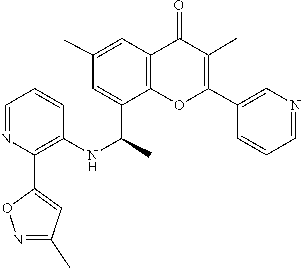 [SMILES: Cc1cc([C@@H](C)Nc2cccnc2-c2cc(C)no2)c2oc(-c3cccnc3)c(C)c(=O)c2c1] | 453 [M + H]⁺ |
| 87C | 3,6-Dimethyl-2-(3- pyridyl)-8-[(1R)-1-[[2- (3-pyridyl)-3-pyridyl]- amino]ethyl]chromen-4- one [EDA-00002-320] | 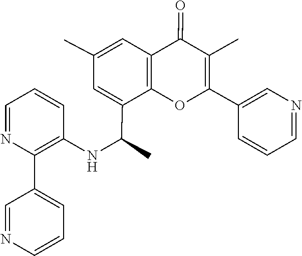 [SMILES: Cc1cc([C@@H](C)Nc2cccnc2-c2cccnc2)c2oc(-c3cccnc3)c(C)c(=O)c2c1] | 449 [M + H]⁺ |
| 88C¹ | 6-Chloro-3-[[(1R)-1-[6- methyl-3-(5-methylisox- azol-4-yl)-4-oxo-2-phen- yl-chromen-8-yl]ethyl]- amino]-N-methylsulfon- yl-pyridine-2-carbox- amide | 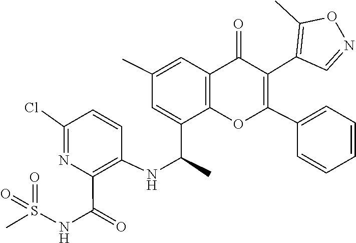 [SMILES: Cc1cc([C@@H](C)Nc2ccc(Cl)nc2C(=O)NS(C)(=O)=O)c2oc(-c3ccccc3)c(-c3cnoc3C)c(=O)c2c1] | 593 [M + H]⁺ |
| 89C¹ | 6-Chloro-3-[(1R)-1-[6- methyl-3-(3-methylisox- azol-4-yl)-4-oxo-2-phen- yl-chromen-8-yl]ethyl]- amino]-N-methylsulfon- yl-pyridine-2-carbox- amide | 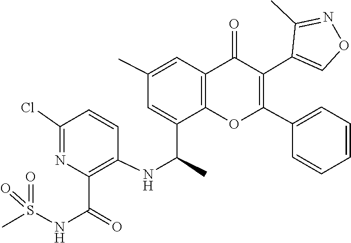 [SMILES: Cc1cc([C@@H](C)Nc2ccc(Cl)nc2C(=O)NS(C)(=O)=O)c2oc(-c3ccccc3)c(-c3conc3C)c(=O)c2c1] | 593 [M + H]⁺ |
| 90C | 5-[3-[[(1R)-1-[3,6- Dimethyl-4-oxo-2-(3- pyridyl)chromen-8-yl]- ethyl]amino]-2-pyridyl]- 1H-pyridin-2-one | 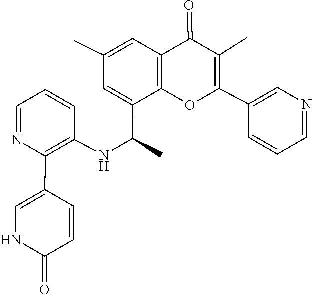 [SMILES: Cc1cc([C@@H](C)Nc2cccnc2-c2ccc(=O)[nH]c2)c2oc(-c3cccnc3)c(C)c(=O)c2c1] | 465 [M + H]⁺ |
| 91C | 8-[(1R)-1-[2-(3-Fluoro- 4-pyridyl)-3-pyridyl]- amino]ethyl]-3,6-dimeth- yl-2-(3-pyridyl)chromen- 4-one | 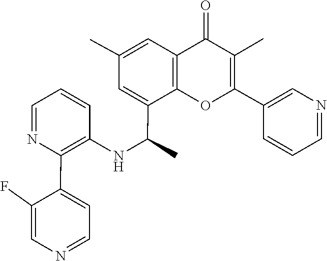 [SMILES: Cc1cc([C@@H](C)Nc2cccnc2-c2ccncc2F)c2oc(-c3cccnc3)c(C)c(=O)c2c1] | 467 [M + H]⁺ |
| 92C¹ | 8-[(1R)-1-[[2-[1-(2- Hydroxy-1,1-dimethyl- ethyl)pyrazol-4-yl]-3- pyridyl]amino]ethyl-3,6- dimethyl-2-(3-pyridyl)- chromen-4-one | 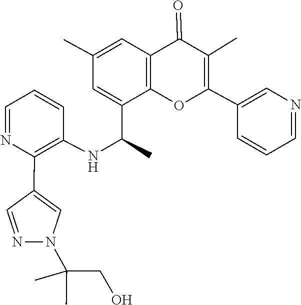 [SMILES: Cc1cc([C@@H](C)Nc2cccnc2-c2cnn(C(C)(C)CO)c2)c2oc(-c3cccnc3)c(C)c(=O)c2c1] | 510 [M + H]⁺ |

¹[1.1′-Bis(di-tert-butylphosphino)ferrocene]dichloropalladium(II) used as catalyst and cesiumcarbonate used as base.

### Example 93C: 8-[(1R)-1-[[2-(3-fluoro-2-pyridyl)-3-pyridyl]amino]ethyl]-3,6-dimethyl-2-(3-pyridyl)chromen-4-one

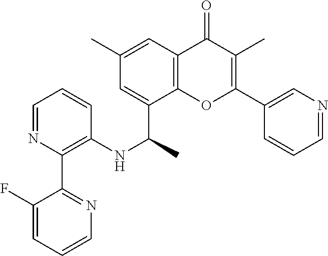 [SMILES: Cc1cc([C@@H](C)Nc2cccnc2-c2ncccc2F)c2oc(-c3cccnc3)c(C)c(=O)c2c1]

A mixture of 8-[(1R)-1-[(2-chloro-3-pyridyl)amino]ethyl]-3,6-dimethyl-2-(3-pyridyl)chromen-4-one (130 mg, 0.32 mmol), copper(I) iodide (24.4 mg, 0.13 mmol), tris(dibenzylideneacetone)dipalladium(0) (29.3 mg, 0.03 mmol), tri-o-tolylphosphine (58.5 mg, 0.19 mmol), and cesium fluoride (97.3 mg, 0.64 mmol) in DMF (0.5 mL) and toluene (1 mL) was treated with tributyl-(3-fluoro-2-pyridyl)stannane (247 mg, 0.64 mmol). The reaction was sparged with argon for 5 min, capped, and heated at 60° C. for 20 h. Temperature was increased to 80° C. for 4 h. Since the reaction was not progressing quickly, added tributyl-(3-fluoro-2-pyridyl)stannane (247 mg, 0.64 mmol), tris(dibenzylideneacetone)dipalladium(0) (29.3 mg, 0.03 mmol), tri-o-tolylphosphine (58.5 mg, 0.19 mmol), copper(I) iodide (24.4 mg, 0.13 mmol), and cesium fluoride (97.3 mg, 0.64 mmol) were added and the reaction stirred at 60° C. over 4 days. Reaction was filtered through diatomaceous earth and the solids washed with DCM (5 mL). The filtrate was concentrated under reduced pressure and the residue purified by silica gel chromatography eluted with 0% to 100% EtOAc in heptane to give an impure product. This material was purified by reversed phase chromatography on C18 eluted with 80% ACN in water (with 0.1% formic acid) to give the title compound (8.6 mg, 6%). ES/MS m/z 467 (M+H).

### Example 76A: 1-[(1R)-1-(3,6-Dimethyl-4-oxo-2-phenyl-chromen-8-yl)ethyl]quinazolin-4-one

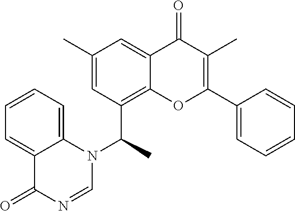 [SMILES: Cc1cc([C@@H](C)n2cnc(=O)c3ccccc32)c2oc(-c3ccccc3)c(C)c(=O)c2c1]

Combined 2-[[(1R)-1-(3,6-dimethyl-4-oxo-2-phenyl-chromen-8-yl)ethyl]amino]benzoic acid (0.20 g, 0.48 mmol), triethyl orthoformate (0.40 mL, 2.4 mmol), and ammonium acetate (0.112 g, 1.45 mmol). The reaction was stirred at 120° C. for 16 h. Concentrated with DCM onto a small amount of diatomaceous earth and purified by reverse phase chromatography to give the title compound (0.061 g, 30%) as a white solid. MS ES+ m/z 423 [M+H]⁺.

### Example 77A: 8-[(1R)-1-(2-Isothiazol-5-ylanilino)ethyl]-3,6-dimethyl-2-phenyl-chromen-4-one

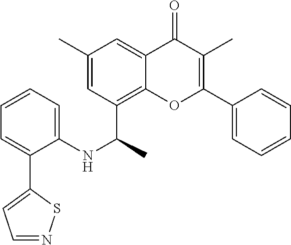 [SMILES: Cc1cc([C@@H](C)Nc2ccccc2-c2ccns2)c2oc(-c3ccccc3)c(C)c(=O)c2c1]

Combined 8-[(1R)-1-(2-bromoanilino)ethyl]-3,6-dimethyl-2-phenyl-chromen-4-one (0.066 g, 0.15 mmol), 5-(4,4,5,5-tetramethyl-1,3,2-dioxaborolan-2-yl)isothiazole (0.062 g, 0.29 mmol), tetrakis(triphenylphosphine)palladium(0) (0.043 g, 0.037 mmol), and cesium carbonate (0.14 g, 0.44 mmol) 1,4-dioxane (2 mL) and water (0.4 mL). Degassed with argon for 1 min and stirred the reaction at 100° C. for 16 hour. Loaded the mixture directly onto a small plug of diatomaceous earth and purified by reverse phase chromatography to give the title compound (0.0078 g, 12%) as a white solid. MS ES+ m/z 453 [M+H]⁺.

The following compound in Table 73 was made in a similar way as described for 8-[(1R)-1-(2-isothiazol-5-ylanilino)ethyl]-3,6-dimethyl-2-phenyl-chromen-4-one.

TABLE 73

| Example | | | MS ES+ |
|---|---|---|---|
| # | Chemical Name | Structure | m/z |
| 78A | 8-[(1R)-1-[(2-Isothiazol- 5-yl-3-pyridyl)amino]- ethyl]-3,6-dimethyl-2- phenyl-chromen-4-one | 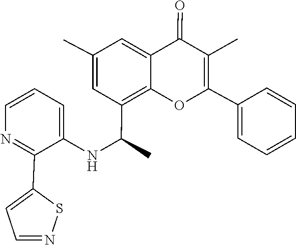 [SMILES: Cc1cc([C@@H](C)Nc2cccnc2-c2ccns2)c2oc(-c3ccccc3)c(C)c(=O)c2c1] | 454 [M + H]⁺ |

The following compounds in Table 74 were made in a similar way as described for N-[[(4R)-2,2-dimethyl-1,3-dioxolan-4-yl]methoxy]-2-[[(1R)-1-(3,6-dimethyl-4-oxo-2-phenyl-chromen-8-yl)ethyl]amino]benzamide. Various methods were used to purify the compounds, which would be apparent to one skilled in the art.

TABLE 74

| Example | | | MS ES+ |
|---|---|---|---|
| # | Chemical Name | Structure | m/z |
| 79A | 3-[[(1R)-1-(3,6-Dimethyl- 4-oxo-2-phenyl-chromen- 8-yl)ethyl]amino]-N- methyl-pyridine-2- carboxamide | 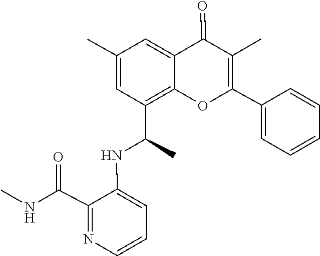 [SMILES: CNC(=O)c1ncccc1N[C@H](C)c1cc(C)cc2c(=O)c(C)c(-c3ccccc3)oc12] | 428 [M + H]⁺ |
| 80A | 3-[[(1R)-1-(3,6-Dimethyl- 4-oxo-2-phenyl-chromen- 8-yl)ethyl]amino]-N- methylsulfonyl-pyridine- 2-carboxamide | 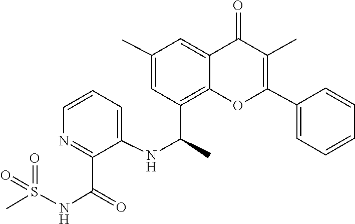 [SMILES: Cc1cc([C@@H](C)Nc2cccnc2C(=O)NS(C)(=O)=O)c2oc(-c3ccccc3)c(C)c(=O)c2c1] | 492 [M + H]⁺ |
| 81A | 2-[[(1R)-1-(3,6-Dimethyl- 4-oxo-2-phenyl-chromen- 8-yl)ethyl]amino]-N- methoxy-benzamide | 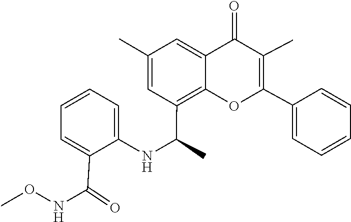 [SMILES: CONC(=O)c1ccccc1N[C@H](C)c1cc(C)cc2c(=O)c(C)c(-c3ccccc3)oc12] | 443 [M + H]⁺ |
| 217A | 3-[[(1R)-1-(3,6-Dimethyl- 4-oxo-2-phenyl-chromen- 8-yl)ethyl]amino]-N-meth- oxy-N-methyl-pyridine-2- carboxamide;2,2,2-tri- fluoroacetic acid | 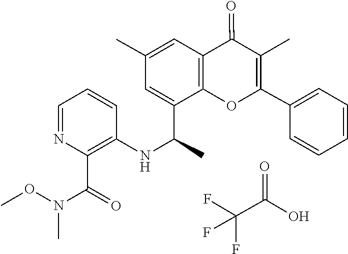 | 458 [M + H]⁺ |

### Example 82A: Methyl 6-bromo-3-[[(1R)-1-(3,6-dimethyl-4-oxo-2-phenyl-chromen-8-yl)ethyl]amino]pyridine-2-carboxylate

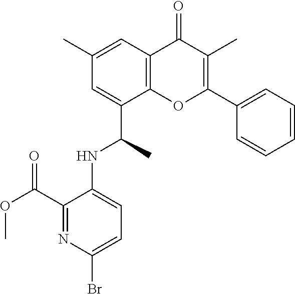 [SMILES: COC(=O)c1nc(Br)ccc1N[C@H](C)c1cc(C)cc2c(=O)c(C)c(-c3ccccc3)oc12]

Combined methyl 6-bromo-3-[[(1R)-1-(2-ethylsulfanyl-3,6-dimethyl-4-oxo-chromen-8-yl)ethyl]amino]pyridine-2-carboxylate (0.050 g, 0.10 mmol), phenylboronic acid (0.016 g, 0.13 mmol), copper(I) thiophene-2-carboxylate (0.039 g, 0.20 mmol), phosphine, tri-2-furanyl- (0.012 g, 0.051 mmol), tris(dibezylideneacetone)dipalladium (0.0093 g, 0.010 mmol), and zinc diacetate (0.0037 g, 0.20 mmol) in 1,4-dioxane (3 mL). Degassed with nitrogen for 5 min and stirred the reaction at 80° C. for 16 h. Added phenylboronic acid (0.016 g, 0.13 mmol), copper(I) thiophene-2-carboxylate (0.039 g, 0.20 mmol), phosphine, tri-2-furanyl- (0.012 g, 0.051 mmol), tris(dibezylideneacetone)dipalladium (0.0093 g, 0.010 mmol), and zinc diacetate (0.0037 g, 0.20 mmol). Degassed with nitrogen for 5 min and stirred the reaction at 100° C. for 16 h. The mixture was filtered over diatomaceous earth. Poured the filtrate into saturated aqueous NH₄Cl with tetramethylethylenediamine (˜1 mL). Extracted with DCM 3×. The combined organic extracts were washed with saturated aqueous NH₄Cl, dried over Na₂SO₄, and concentrated. The crude product was purified by reverse phase chromatography eluted with a gradient of 10 to 10000 ACN in aqueous NH₄HCO₃ (10 mM, plus 500 MeOH) to give the title compound (0.026 g, 490%) as a white solid. MS ES+ m/z 507 [M+H]⁺.

The following compounds in Table 75 were made in a similar way as described for 2-[[(1R)-1-[2-(2-fluorophenyl)-3,6-dimethyl-4-oxo-chromen-8-yl]ethyl]amino]benzonitrile. Various methods were used to purify the compounds, which would be apparent to one skilled in the art.

TABLE 75

| Example | | | MS ES+ |
|---|---|---|---|
| # | Chemical Name | Structure | m/z |
| 83A | 2-[[(1R)-1-[3,6-Dimethyl-4-oxo- 2-(3-pyridyl)chromen-8- yl]ethyl]amino]-N-methyl- benzamide | 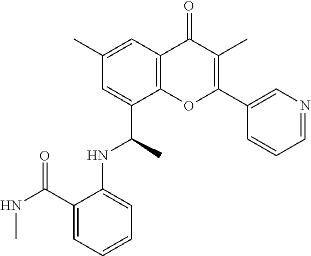 [SMILES: CNC(=O)c1ccccc1N[C@H](C)c1cc(C)cc2c(=O)c(C)c(-c3cccnc3)oc12] | 428 [M + H]⁺ |
| 84A | 2-[[(1R)-1-[3,6-Dimethyl-2-(2- methylindazol-5-yl)-4-oxo- chromen-8-ylethyl]amino]-N- methyl-benzamide | 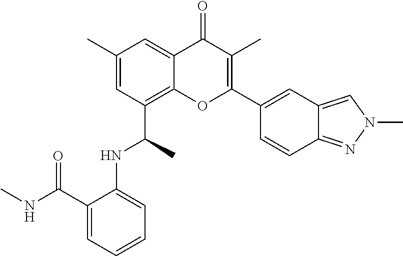 | 481 [M + H]⁺ |
| 85A | 2-[[(1R)-1-[2-(2-Fluorophenyl)- 3,6-dimethyl-4-oxo-chromen-8- yl]ethyl]amino]-N-methyl- benzamide | 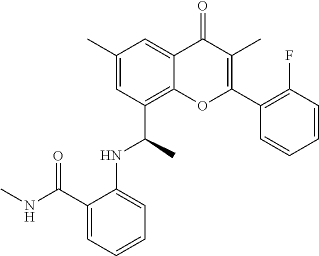 [SMILES: CNC(=O)c1ccccc1N[C@H](C)c1cc(C)cc2c(=O)c(C)c(-c3ccccc3F)oc12] | 445 [M + H]⁺ |
| 86A | 2-[[(1R)-1-[2-(3-Fluorophenyl)- 3,6-dimethyl-4-oxo-chromen-8- yl]ethyl]amino]-N-methyl- benzamide | 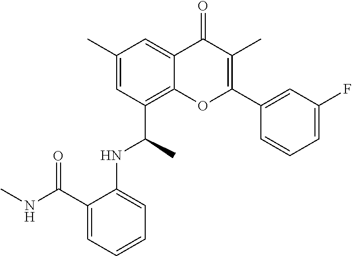 [SMILES: CNC(=O)c1ccccc1N[C@H](C)c1cc(C)cc2c(=O)c(C)c(-c3cccc(F)c3)oc12] | 445 [M + H]⁺ |
| 87A | 2-[[(1R)-1-[2-(3-Cyanophenyl)- 3,6-dimethyl-4-oxo-chromen-8- yl]ethyl]amino]-N-methyl- benzamide | 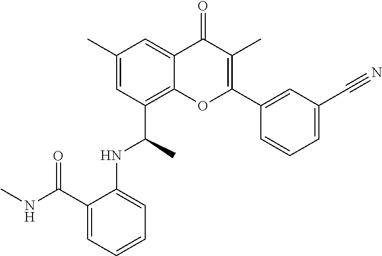 [SMILES: CNC(=O)c1ccccc1N[C@H](C)c1cc(C)cc2c(=O)c(C)c(-c3cccc(C#N)c3)oc12] | 452 [M + H]⁺ |
| 88A | Methyl 3-[3,6-dimethyl-8-[(1R)- 1-[2- (methylcarbamoyl)anilino]ethyl]- 4-oxo-chromen-2-yl]benzoate | 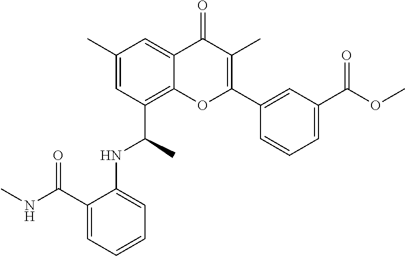 | 485 [M + H]⁺ |
| 89A | Methyl 6-chloro-3-[[(1R)-1-[3,6- dimethyl-4-oxo-2-(3- pyridyl)chromen-8- yl]ethyl]amino]pyridine-2- carboxylate | 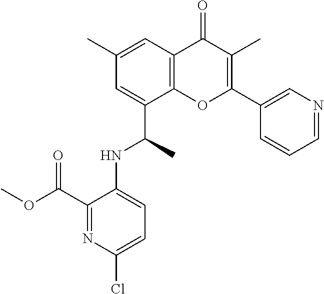 [SMILES: COC(=O)c1nc(Cl)ccc1N[C@H](C)c1cc(C)cc2c(=O)c(C)c(-c3cccnc3)oc12] | 464 [M + H]⁺ |
| 90A | 3,6-Dimethyl-2-phenyl-8-[(1R)- 1-[[2-(trifluoromethyl)-3- pyridyl]amino]ethyl]chromen-4- one | 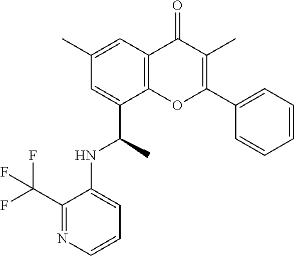 [SMILES: Cc1cc([C@@H](C)Nc2cccnc2C(F)(F)F)c2oc(-c3ccccc3)c(C)c(=O)c2c1] | 439 [M + H]⁺ |

### Example 94C: 6-Chloro-3-[[(1R)-1-[3,6-dimethyl-2-(1-methylpyrazolo[3,4-b]pyridin-5-yl)-4-oxo-chromen-8-yl]ethyl]amino]-N-methylsulfonyl-pyridine-2-carboxamide

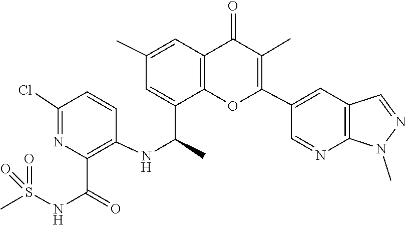 [SMILES: Cc1cc([C@@H](C)Nc2ccc(Cl)nc2C(=O)NS(C)(=O)=O)c2oc(-c3cnc4c(cnn4C)c3)c(C)c(=O)c2c1]

Combined 6-chloro-3-[[(1R)-1-(2-ethylsulfanyl-3,6-dimethyl-4-oxo-chromen-8-yl)ethyl]amino]-N-methylsulfonyl-pyridine-2-carboxamide (48 mg, 0.09 mmol), 1-methyl-5-(4,4,5,5-tetramethyl-1,3,2-dioxaborolan-2-yl)pyrazolo[3,4-b]pyridine (73 mg, 0.28 mmol), copper(I) thiophene-2-carboxylate (27 mg, 0.14 mmol), and [1,1′-bis(diphenylphosphino)ferrocene]dichloropalladium(II) (6.9 mg, 0.09 mmol) in EtOH (0.9 mL) degassed with argon for 5 min. The reaction was flushed with argon and stirred at 65° C. for 17 h. Added DCM and water to the cooled reaction and separated the layers with a phase separator. The aqueous layer was extracted with DCM. The combined organic layers were concentrated under reduced pressure and the residue purified by reversed phase chromatography eluted with 10% to 100% ACN in 10 mM aqueous NH₄HCO₃ (with 5% MeOH) to give the title compound (13.3 mg, 24%). ES/MS m/z 581 (M+H).

The following compound in Table 76 was made in a similar way as described for 6-chloro-3-[[(1R)-1-[3,6-dimethyl-2-(1-methylpyrazolo[3,4-b]pyridin-5-yl)-4-oxo-chromen-8-yl]ethyl]amino]-N-methylsulfonyl-pyridine-2-carboxamide. Various methods were used to purify the compound, which would be apparent to one skilled in the art.

TABLE 76

| Example | | | MS ES+ |
|---|---|---|---|
| # | Chemical Name | Structure | m/z |
| 95C¹ | 6-Chloro-3- [[(1R)-1-[2-[3- (1-hydroxy-1- methyl- ethyl)phenyl]- 3,6-dimethyl-4- oxo-chromen-8- yl]ethyl]amino]- N- methylsulfonyl- pyridine-2- carboxamide | 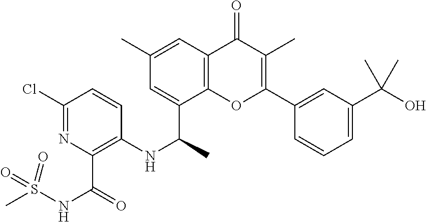 [SMILES: Cc1cc([C@@H](C)Nc2ccc(Cl)nc2C(=O)NS(C)(=O)=O)c2oc(-c3cccc(C(C)(C)O)c3)c(C)c(=O)c2c1] | 584 [M + H]⁺ |

¹Cesium carbonate added to this reaction.

### Example 91A: 3,6-Dimethyl-8-[(1R)-1-(2-nitroanilino)ethyl]-2-phenyl-chromen-4-one

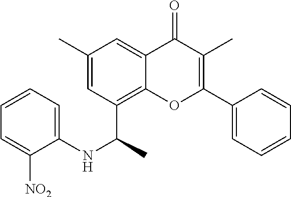 [SMILES: Cc1cc([C@@H](C)Nc2ccccc2[N+](=O)[O-])c2oc(-c3ccccc3)c(C)c(=O)c2c1]

To a solution of 8-[(1R)-1-aminoethyl]-3,6-dimethyl-2-phenyl-chromen-4-one (0.500 g, 1.70 mmol) in ACN (8 mL) was added potassium carbonate (0.589 g, 4.26 mmol) and 1-fluoro-2-nitrobenzene (0.361 g, 2.56 mmol). The reaction was stirred at 80° C. overnight, filtered, and the solids were washed with DCM. The filtrate was concentrated and purified by silica gel chromatography eluted with a gradient of 0% to 100% EtOAc in hexanes. Fractions with the desired product were combined and concentrated, and the residue was recrystallized from DCM/hexanes to give the title compound (0.675 g, 96%) as a yellow solid. MS ES+ m/z 415 [M+H]⁺.

### Example 92A: N-[2-[[(1R)-1-(3,6-Dimethyl-4-oxo-2-phenyl-chromen-8-yl)ethyl]amino]phenyl]methanesulfonamide

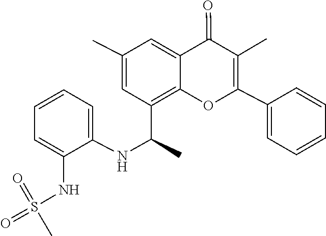 [SMILES: Cc1cc([C@@H](C)Nc2ccccc2NS(C)(=O)=O)c2oc(-c3ccccc3)c(C)c(=O)c2c1]

To a suspension of 3,6-dimethyl-8-[(1R)-1-(2-nitroanilino)ethyl]-2-phenyl-chromen-4-one (0.675 g, 1.63 mmol) and iron (0.273 g, 4.89 mmol) in ethanol (8 mL) was added saturated aqueous NH₄Cl (8 mL). The reaction was stirred at 75° C. for 30 min, filtered through diatomaceous earth, and the solids were washed with DCM and MeOH. The filtrate was concentrated to remove most of the alcohols. The remaining liquids were extracted with DCM. The organic extracts were dried over MgSO₄, filtered, and concentrated. The residue was purified by silica gel chromatography eluted with a gradient of 0 to 100% EtOAc in hexanes to give 8-[(1R)-1-(2-aminoanilino)ethyl]-3,6-dimethyl-2-phenyl-chromen-4-one (0.560 g, 89%). MS ES+ m/z 385 [M+H]⁺. To a portion of the intermediate aniline (0.050 g, 0.13 mmol) in pyridine (1 mL) at 0° C. was added a solution of methanesulfonyl chloride (0.011 mL, 0.14 mmol) in DCM (0.5 mL). After 30 min, diluted with EtOAc and 1N aqueous HCl. Extracted the aqueous layer twice more with EtOAc. The combined organic extracts were dried over Na₂SO₄, filtered, concentrated, and the residue was recrystallized from DCM/hexanes to give the title compound (0.040 g, 66%). MS ES+ m/z 463 [M+H]⁺.

### Example 93A: 1-[3-[[(1R)-1-(3,6-Dimethyl-4-oxo-2-phenyl-chromen-8-yl)ethyl]amino]-2-pyridyl]pyrrolidin-2-one

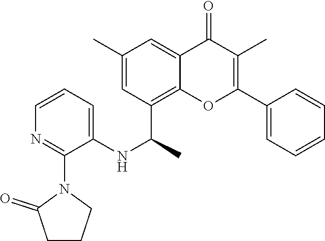 [SMILES: Cc1cc([C@@H](C)Nc2cccnc2N2CCCC2=O)c2oc(-c3ccccc3)c(C)c(=O)c2c1]

A vial containing cuprous iodide (0.003 g, 0.02 mmol) and potassium carbonate (0.034 g, 0.25 mmol) in toluene (1 mL) was degassed and purged with nitrogen. Pyrrolidin-2-one (0.016 g, 0.19 mmol), methyl[2-(methylamino)ethyl]amine (0.0022 g, 0.025 mmol), and 8-[(1R)-1-[(2-chloro-3-pyridyl)amino]ethyl]-3,6-dimethyl-2-phenyl-chromen-4-one (0.050 g, 0.12 mmol) were added. The reaction was sealed and heated at 120° C. overnight. The mixture was concentrated and purified by reverse phase chromatography eluted with a gradient of 0% to 100% ACN in H₂O+0.1% formic acid. Fractions containing the desired product were combined, washed with saturated aqueous sodium chloride, and extracted with IPA:CHCl₃ (1:3). The combined extracts were dried over MgSO₄, filtered, and concentrated. The material was recrystallized from a mixture of DCM and hexanes to give the title compound (0.010 g, 19%) as a white solid. MS ES+ m/z 454 [M+H]⁺.

### Example 94A: N-[2-[1-(3,6-Dimethyl-4-oxo-2-phenyl-chromen-8-yl)ethylamino]phenyl]sulfonylacetamide

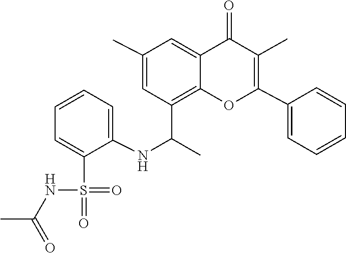 [SMILES: CC(=O)NS(=O)(=O)c1ccccc1NC(C)c1cc(C)cc2c(=O)c(C)c(-c3ccccc3)oc12]

To a solution of 2-[1-(3,6-dimethyl-4-oxo-2-phenyl-chromen-8-yl)ethylamino]benzenesulfonamide (0.070 g, 0.16 mmol) in pyridine (1 mL) was added acetic anhydride (0.044 mL, 0.47 mmol) and 4-dimethylaminopyridine (0.0057 g, 0.047 mmol). The reaction was stirred at rt overnight and concentrated. Diluted with toluene and concentrated 2×. Purified by reverse phase chromatography to give the title compound (0.039 g, 51%). MS ES+ m/z 491 [M+H]⁺.

### Example 95A and Example 96A: N-[2-[1-(3,6-Dimethyl-4-oxo-2-phenyl-chromen-8-yl)ethylamino]phenyl]sulfonylacetamide, Isomer 1 and Isomer 2

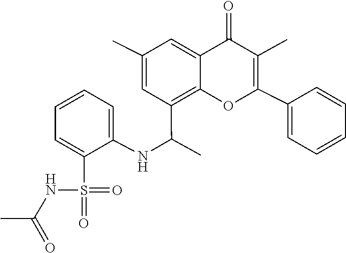 [SMILES: CC(=O)NS(=O)(=O)c1ccccc1NC(C)c1cc(C)cc2c(=O)c(C)c(-c3ccccc3)oc12]

N-[2-[1-(3,6-Dimethyl-4-oxo-2-phenyl-chromen-8-yl)ethylamino]phenyl]sulfonylacetamide (0.035 g) was separated by chiral SFC [Chiralpak AS-H, 21×150 mm; 20% EtOH in CO₂] to give the title compounds (Isomer 1, first-eluting; 0.010 g, >98% ee) and (Isomer 2; 0.018 g, >98% ee). MS ES+ m/z 491 [M+H]⁺.

### Example 97A: N-[(2R)-2,3-Dihydroxypropoxy]-2-[[(1R)-1-(3,6-dimethyl-4-oxo-2-phenyl-chromen-8-yl)ethyl]amino]benzamide

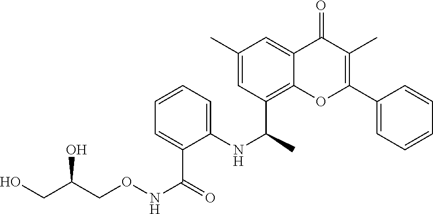 [SMILES: Cc1cc([C@@H](C)Nc2ccccc2C(=O)NOC[C@H](O)CO)c2oc(-c3ccccc3)c(C)c(=O)c2c1]

To a solution of N-[[(4R)-2,2-dimethyl-1,3-dioxolan-4-yl]methoxy]-2-[[(1R)-1-(3,6-dimethyl-4-oxo-2-phenyl-chromen-8-yl)ethyl]amino]benzamide (0.171 g, 0.315 mmol) in MeOH (2 mL) was added p-toluenesulfonic acid monohydrate (0.012 g, 0.063 mmol). The reaction stirred at rt overnight. The mixture was concentrated and purified by silica gel chromatography eluted with 0% to 100% EtOAc:EtOH (3:1) in heptane. Fractions with the desired product were combined and concentrated, and the residue was recrystallized from MTBE/hexanes to give the title compound (0.061 g, 39%) as a white solid. MS ES+ m/z 503 [M+H]⁺.

### Example 98A: Methyl N-[2-[[(1R)-1-(3,6-dimethyl-4-oxo-2-phenyl-chromen-8-yl)ethyl]amino]phenyl]carbamate

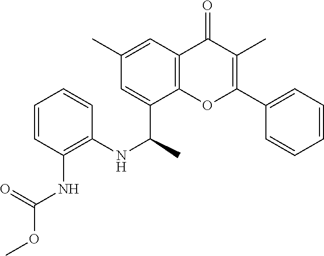 [SMILES: COC(=O)Nc1ccccc1N[C@H](C)c1cc(C)cc2c(=O)c(C)c(-c3ccccc3)oc12]

To a solution of 8-[(1R)-1-(2-aminoanilino)ethyl]-3,6-dimethyl-2-phenyl-chromen-4-one (0.100 g, 0.260 mmol) and DIPEA (0.068 mL, 0.390 mmol) in DCM (1.5 mL) was added methyl carbonochloridate (0.030 mL, 0.390 mmol). After 1 h, purified by silica gel chromatography eluted with 0% to 100% EtOAc in heptane. Fractions with the desired product were combined, concentrated, and purified by reverse phase chromatography eluted with a gradient of 10 to 100% ACN in H₂O+0.1% formic acid to give the title compound (0.043 g, 37%) after lyophilization. MS ES+ m/z 443 [M+H]⁺.

### Example 99A: 2-[[(1R)-1-(3,6-Dimethyl-4-oxo-2-phenyl-chromen-8-yl)ethyl]amino]-N-methyl-benzenesulfonamide

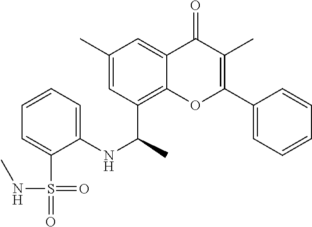 [SMILES: CNS(=O)(=O)c1ccccc1N[C@H](C)c1cc(C)cc2c(=O)c(C)c(-c3ccccc3)oc12]

A vial containing cuprous iodide (0.0065 g, 0.034 mmol) and cesium carbonate (0.222 g, 0.682 mmol) in DMSO (2 mL) was degassed and purged with nitrogen. 8-[(1R)-1-Aminoethyl]-3,6-dimethyl-2-phenyl-chromen-4-one (0.100 g, 0.341 mmol), 2-bromo-N-methyl benzenesulfonamide (0.102 g, 0.409 mmol) and o-phenanthroline (0.012 g, 0.068 mmol) were added. The reaction was sealed and heated at 150° C. for 30 min. Diluted with water and extracted twice with EtOAc. The combined organic extracts were dried over Na₂SO₄, filtered, and concentrated. The material was purified by silica gel chromatography eluted with 0% to 100% EtOAc in hexanes to give the title compound (0.035 g, 22%) as a white solid. MS ES+ m/z 463 [M+H]⁺.

The following compound in Table 77 was made in a similar way as described for 2-[[(1R)-1-(3,6-dimethyl-4-oxo-2-phenyl-chromen-8-yl)ethyl]amino]-N-methyl-benzenesulfonamide. Various methods were used to purify the compound, which would be apparent to one skilled in the art.

TABLE 77

| Example | | | MS ES+ |
|---|---|---|---|
| # | Chemical Name | Structure | m/z |
| 96C | 8-[(1R)-1-[2-(1- Hydroxy-1-methyl- ethyl)anilino]ethyl]- 3,6-dimethyl-2- phenyl-chromen-4- one | 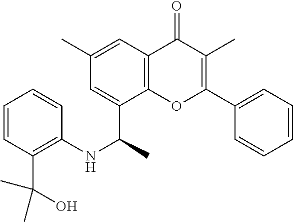 [SMILES: Cc1cc([C@@H](C)Nc2ccccc2C(C)(C)O)c2oc(-c3ccccc3)c(C)c(=O)c2c1] | 428 [M + H]⁺ |

### Example 100A: 2-[[(1R)-1-(3,6-Dimethyl-4-oxo-2-phenyl-chromen-8-yl)ethyl]amino]-N-methylsulfonyl-benzamide

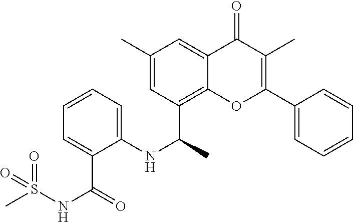 [SMILES: Cc1cc([C@@H](C)Nc2ccccc2C(=O)NS(C)(=O)=O)c2oc(-c3ccccc3)c(C)c(=O)c2c1]

Combined 2-[[(1R)-1-(3,6-dimethyl-4-oxo-2-phenyl-chromen-8-yl)ethyl]amino]benzoic acid (0.200 g, 0.484 mmol), 2-chloro-1-methyl-pyridin-1-ium iodide (0.148 g, 0.580 mmol), methylsulfonamide (0.092 g, 0.967 mmol), and 4-dimethylaminopyridine (0.0059 g, 0.048 mmol) in DCM (4 mL). After 5 min, triethylamine (0.20 mL, 1.45 mmol) was added. The reaction was stirred at rt for 1 h. Diluted with DCM and washed with 1N aqueous HCl. The aqueous layer was extracted twice with DCM. The combined organic extracts were dried over MgSO₄, filtered, and concentrated. The residue was purified by reverse phase chromatography eluted with a gradient of 0% to 100% ACN in H₂O+0.1% formic acid. Fractions containing the desired product were combined, washed with saturated aqueous sodium chloride, and extracted with IPA:CHCl₃ (1:3). The combined extracts were dried over MgSO₄, filtered, and concentrated. The material was recrystallized from a mixture of DCM and hexanes to give the title compound (0.0044 g, 1.9%) as a white solid. MS ES+ m/z 491 [M+H]⁺.

The following compounds in Table 78 were made in a similar way as described for 2-[[(1R)-1-(3,6-dimethyl-4-oxo-2-phenyl-chromen-8-yl)ethyl]amino]-N-methylsulfonyl-benzamide. Various methods were used to purify the compounds, which would be apparent to one skilled in the art.

TABLE 78

| Example | | | MS ES+ |
|---|---|---|---|
| # | Chemical Name | Structure | m/z |
| 218A | 6-Chloro-3-[[(1R)-1-(3,6- dimethyl-4-oxo-2-phenyl- chromen-8-yl)ethyl]amino]-N- methylsulfonyl-pyridine-2- carboxamide | 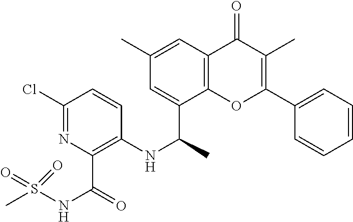 [SMILES: Cc1cc([C@@H](C)Nc2ccc(Cl)nc2C(=O)NS(C)(=O)=O)c2oc(-c3ccccc3)c(C)c(=O)c2c1] | 526 [M + H]⁺ |
| 219A | 6-Chloro-3-[[(1R)-1-[2-(3,4- difluorophenyl)-3,6-dimethyl-4- oxo-chromen-8-yl]ethyl]amino]- N-methylsulfonyl-pyridine-2- carboxamide | 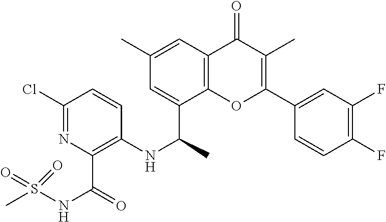 [SMILES: Cc1cc([C@@H](C)Nc2ccc(Cl)nc2C(=O)NS(C)(=O)=O)c2oc(-c3ccc(F)c(F)c3)c(C)c(=O)c2c1] | 562 [M + H]⁺ |
| 220A | 6-Chloro-3-[[(1R)-1-[2-(2- fluorophenyl)-3,6-dimethyl-4- oxo-chromen-8-yl]ethyl]amino]- N-methylsulfonyl-pyridine-2- carboxamide | 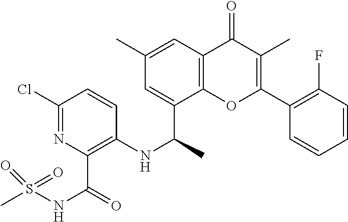 [SMILES: Cc1cc([C@@H](C)Nc2ccc(Cl)nc2C(=O)NS(C)(=O)=O)c2oc(-c3ccccc3F)c(C)c(=O)c2c1] | 544 [M + H]⁺ |
| 221A | 6-Chloro-3-[[(1R)-1-[3,6- dimethyl-2-(1-methylpyrazol-4- yl)-4-oxo-chromen-8- yl]ethyl]amino]-N- methylsulfonyl-pyridine-2- carboxamide | 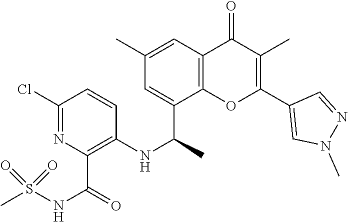 [SMILES: Cc1cc([C@@H](C)Nc2ccc(Cl)nc2C(=O)NS(C)(=O)=O)c2oc(-c3cnn(C)c3)c(C)c(=O)c2c1] | 530 [M + H]⁺ |
| 222A | 6-Chloro-3-[[(1R)-1-[3,6- dimethyl-2-(2-methylindazol-5- yl)-4-oxo-chromen-8- ylethyl]amino]-N-methylsulfonyl- pyridine-2-carboxamide | 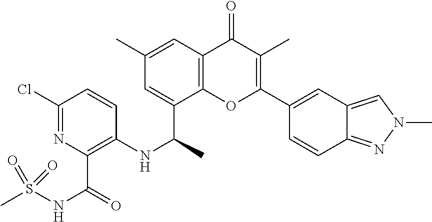 | 580 [M + H]⁺ |
| 223A | 6-Chloro-N- (difluoromethylsulfonyl)-3- [[(1R)-1-(3,6-dimethyl-4-oxo-2- phenyl-chromen-8- yl)ethyl]amino]pyridine-2- carboxamide | 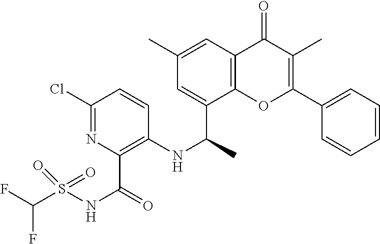 [SMILES: Cc1cc([C@@H](C)Nc2ccc(Cl)nc2C(=O)NS(=O)(=O)C(F)F)c2oc(-c3ccccc3)c(C)c(=O)c2c1] | 562 [M + H]⁺ |
| 224A | 6-Chloro-3-[[(1R)-1-[2-(3- cyanophenyl)-3,6-dimethyl-4- oxo-chromen-8-ylethyl]amino]- N-methylsulfonyl-pyridine-2- carboxamide | 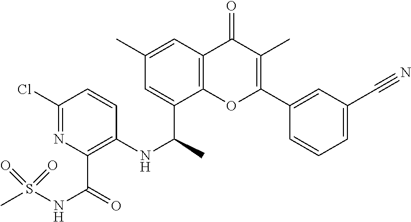 [SMILES: Cc1cc([C@@H](C)Nc2ccc(Cl)nc2C(=O)NS(C)(=O)=O)c2oc(-c3cccc(C#N)c3)c(C)c(=O)c2c1] | 551 [M + H]⁺ |
| 225A | 8-[(1R)-1-[[6-Chloro-2-(1,1- dioxo-1,2-thiazolidine-2- carbonyl)-3-pyridyl]amino]ethyl]- 3,6-dimethyl-2-phenyl-chromen- 4-one | 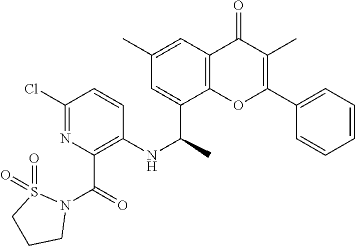 [SMILES: Cc1cc([C@@H](C)Nc2ccc(Cl)nc2C(=O)N2CCCS2(=O)=O)c2oc(-c3ccccc3)c(C)c(=O)c2c1] | 552 [M + H]⁺ |
| 226A | N-(Difluoromethylsulfonyl)-3- [[(1R)-1-(3,6-dimethyl-4-oxo-2- phenyl-chromen-8- yl)ethyl]amino]pyridine-2- carboxamide | 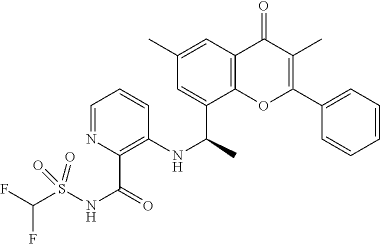 [SMILES: Cc1cc([C@@H](C)Nc2cccnc2C(=O)NS(=O)(=O)C(F)F)c2oc(-c3ccccc3)c(C)c(=O)c2c1] | 528 [M + H]⁺ |
| 227A | 6-[[(1R)-1-(3,6-Dimethyl-4-oxo- 2-phenyl-chromen-8- yl)ethyl]amino]-2,3-difluoro-N- methylsulfonyl-benzamide | 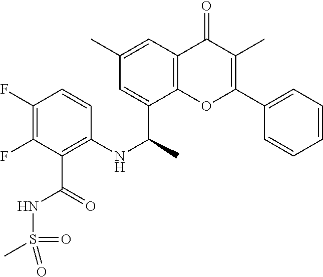 [SMILES: Cc1cc([C@@H](C)Nc2ccc(F)c(F)c2C(=O)NS(C)(=O)=O)c2oc(-c3ccccc3)c(C)c(=O)c2c1] | 527 [M + H]⁺ |
| 228A | 8-[(1R)-1-[[2-(1,1-Dioxo-1,2- thiazolidine-2-carbonyl)-3- pyridyl]amino]ethyl]-3,6- dimethyl-2-phenyl-chromen-4- one | 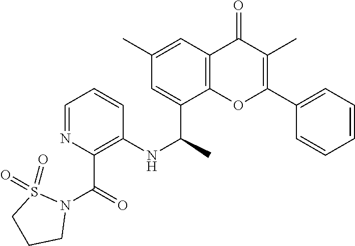 [SMILES: Cc1cc([C@@H](C)Nc2cccnc2C(=O)N2CCCS2(=O)=O)c2oc(-c3ccccc3)c(C)c(=O)c2c1] | 518 [M + H]⁺ |
| 229A | 3-[[(1R)-1-(3,6-Dimethyl-4-oxo-2- phenyl-chromen-8- yl)ethyl]amino]-N-(2,2,2- trifluoroethylsulfonyl)pyridine-2- carboxamide | 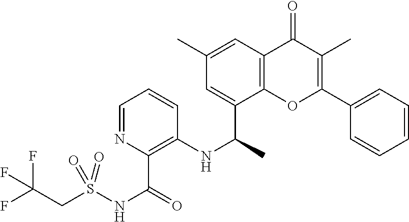 [SMILES: Cc1cc([C@@H](C)Nc2cccnc2C(=O)NS(=O)(=O)CC(F)(F)F)c2oc(-c3ccccc3)c(C)c(=O)c2c1] | 560 [M + H]⁺ |
| 230A | 3-[[(1R)-1-(3,6-Dimethyl-4-oxo- 2-phenyl-chromen-8- yl)ethyl]amino]-N- (trifluoromethylsulfonyl)pyridine- 2-carboxamide | 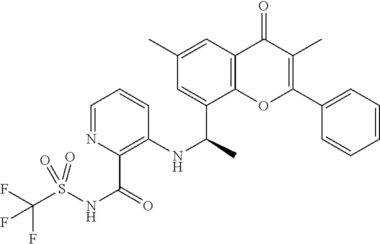 [SMILES: Cc1cc([C@@H](C)Nc2cccnc2C(=O)NS(=O)(=O)C(F)(F)F)c2oc(-c3ccccc3)c(C)c(=O)c2c1] | 546 [M + H]⁺ |
| 231A | 2-[[(1R)-1-(3,6-Dimethyl-4-oxo-2- phenyl-chromen-8- yl)ethyl]amino]-6-fluoro-N- methylsulfonyl-benzamide | 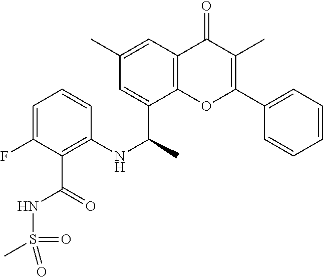 [SMILES: Cc1cc([C@@H](C)Nc2cccc(F)c2C(=O)NS(C)(=O)=O)c2oc(-c3ccccc3)c(C)c(=O)c2c1] | 509 [M + H]⁺ |
| 232A | N-Cyclopropylsulfonyl-3-[[(1R)- 1-(3,6-dimethyl-4-oxo-2-phenyl- chromen-8- yl)ethyl]amino]pyridine-2- carboxamide | 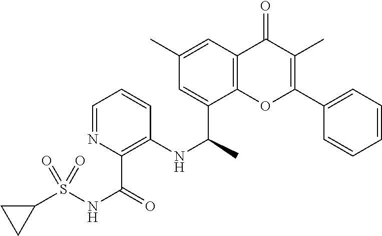 [SMILES: Cc1cc([C@@H](C)Nc2cccnc2C(=O)NS(=O)(=O)C2CC2)c2oc(-c3ccccc3)c(C)c(=O)c2c1] | 518 [M + H]⁺ |
| 233A | 3-[[(1R)-1-(3,6-Dimethyl-4-oxo-2- phenyl-chromen-8- yl)ethyl]amino]-6-methyl-N- methylsulfonyl-pyridine-2- carboxamide | 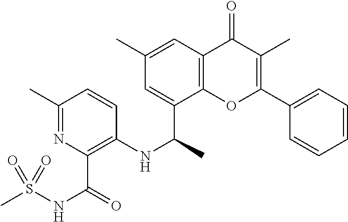 [SMILES: Cc1cc([C@@H](C)Nc2ccc(C)nc2C(=O)NS(C)(=O)=O)c2oc(-c3ccccc3)c(C)c(=O)c2c1] | 506 [M + H]⁺ |
| 234A | 3-[[(1R)-1-[3,6-Dimethyl-4-oxo- 2-(3-pyridyl)chromen-8- yl]ethyl]amino]-N- methylsulfonyl-pyridine-2- carboxamide | 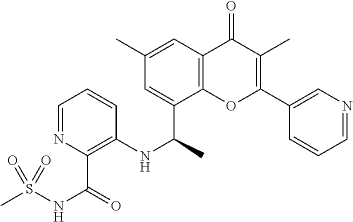 [SMILES: Cc1cc([C@@H](C)Nc2cccnc2C(=O)NS(C)(=O)=O)c2oc(-c3cccnc3)c(C)c(=O)c2c1] | 493 [M + H]⁺ |
| 235A | N-(Azetidin-1-ylsulfonyl)-3- [[(1R)-1-(3,6-dimethyl-4-oxo-2- phenyl-chromen-8- yl)ethyl]amino]pyridine-2- carboxamide | 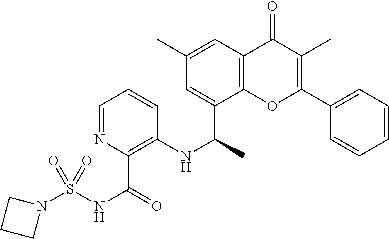 [SMILES: Cc1cc([C@@H](C)Nc2cccnc2C(=O)NS(=O)(=O)N2CCC2)c2oc(-c3ccccc3)c(C)c(=O)c2c1] | 533 [M + H]⁺ |
| 236A | 3-[[(1R)-1-[2-[6- (Difluoromethyl)-2-pyridyl]-3,6- dimethyl-4-oxo-chromen-8- yl]ethyl]amino]-N- methylsulfonyl-pyridine-2- carboxamide | 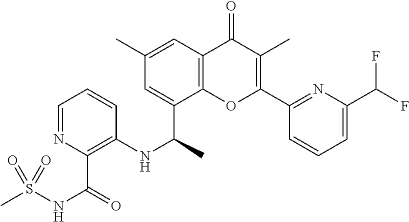 [SMILES: Cc1cc([C@@H](C)Nc2cccnc2C(=O)NS(C)(=O)=O)c2oc(-c3cccc(C(F)F)n3)c(C)c(=O)c2c1] | 543 [M + H]⁺ |
| 237A | 3-[[(1R)-1-(3,6-Dimethyl-4-oxo-2- phenyl-chromen-8- yl)ethyl]amino]-N- morpholinosulfonyl-pyridine-2- carboxamide | 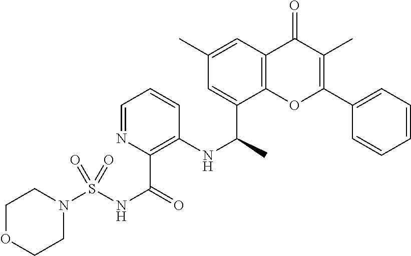 [SMILES: Cc1cc([C@@H](C)Nc2cccnc2C(=O)NS(=O)(=O)N2CCOCC2)c2oc(-c3ccccc3)c(C)c(=O)c2c1] | 563 [M + H]⁺ |
| 238A | N-Cyano-2-[[(1R)-1-(3,6- dimethyl-4-oxo-2-phenyl- chromen-8- yl)ethyl]amino]benzamide | 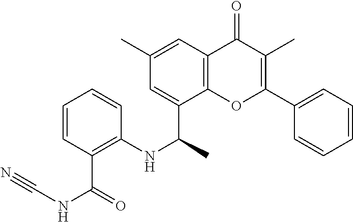 [SMILES: Cc1cc([C@@H](C)Nc2ccccc2C(=O)NC#N)c2oc(-c3ccccc3)c(C)c(=O)c2c1] | 438 [M + H]+ |
| 239A | 3-[[(1R)-1-(3,6-Dimethyl-4-oxo-2- phenyl-chromen-8- yl)ethyl]amino]-N-methyl-N- methylsulfonyl-pyridine-2- carboxamide | 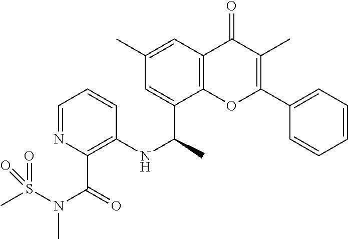 [SMILES: Cc1cc([C@@H](C)Nc2cccnc2C(=O)N(C)S(C)(=O)=O)c2oc(-c3ccccc3)c(C)c(=O)c2c1] | 506 [M + H]⁺ |
| 240A | 2-[[(1R)-1-[3,6-Dimethyl-4-oxo-2- (3-pyridyl)chromen-8- yl]ethyl]amino]-N- methylsulfonyl-benzamide | 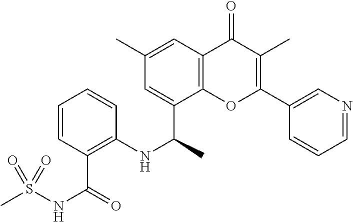 [SMILES: Cc1cc([C@@H](C)Nc2ccccc2C(=O)NS(C)(=O)=O)c2oc(-c3cccnc3)c(C)c(=O)c2c1] | 492 [M + H]⁺ |
| 241A | 6-Chloro-3-[[(1R)-1-[2-[1- (difluoromethyl)pyrazol-4-yl]- 3,6-dimethyl-4-oxo-chromen-8- yl]ethyl]amino]-N- methylsulfonyl-pyridine-2- carboxamide | 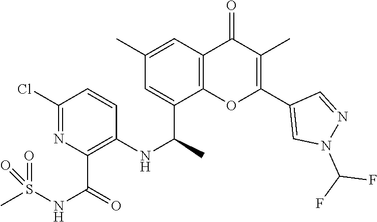 [SMILES: Cc1cc([C@@H](C)Nc2ccc(Cl)nc2C(=O)NS(C)(=O)=O)c2oc(-c3cnn(C(F)F)c3)c(C)c(=O)c2c1] | 566 [M + H]⁺ |
| 242A | 6-Chloro-3-[[(1R)-1-[2-(3-cyano- 2-fluoro-phenyl)-3,6-dimethyl-4- oxo-chromen-8-yl]ethyl]amino]- N-methylsulfonyl-pyridine-2- carboxamide | 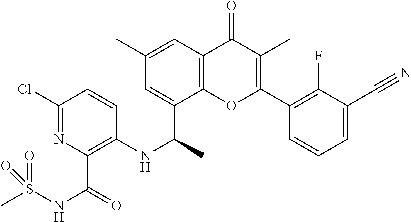 [SMILES: Cc1cc([C@@H](C)Nc2ccc(Cl)nc2C(=O)NS(C)(=O)=O)c2oc(-c3cccc(C#N)c3F)c(C)c(=O)c2c1] | 569 [M + H]⁺ |
| 243A | 6-Chloro-3-[[(1R)-1-[2-(1- isopropylpyrazol-4-yl)-3,6- dimethyl-4-oxo-chromen-8- yl]ethyl]amino]-N- methylsulfonyl-pyridine-2- carboxamide | 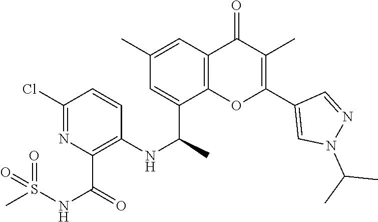 [SMILES: Cc1cc([C@@H](C)Nc2ccc(Cl)nc2C(=O)NS(C)(=O)=O)c2oc(-c3cnn(C(C)C)c3)c(C)c(=O)c2c1] | 558 [M + H]⁺ |
| 244A | 6-Chloro-3-[[(1R)-1-[3-methyl-2- (1-methylpyrazol-4-yl)-4-oxo-6- (trifluoromethyl)chromen-8- yl]ethyl]amino]-N- methylsulfonyl-pyridine-2- carboxamide | 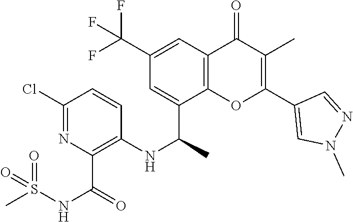 [SMILES: Cc1c(-c2cnn(C)c2)oc2c([C@@H](C)Nc3ccc(Cl)nc3C(=O)NS(C)(=O)=O)cc(C(F)(F)F)cc2c1=O] | 584 [M + H]⁺ |
| 245A | 6-Chloro-3-[[(1R)-1-[3,6- dimethyl-2-(1-methylpyrazol-3- yl)-4-oxo-chromen-8- yl]ethyl]amino]-N- methylsulfonyl-pyridine-2- carboxamide | 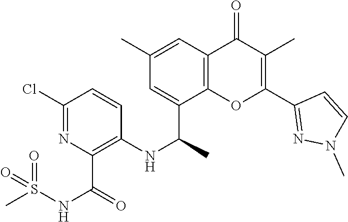 [SMILES: Cc1cc([C@@H](C)Nc2ccc(Cl)nc2C(=O)NS(C)(=O)=O)c2oc(-c3ccn(C)n3)c(C)c(=O)c2c1] | 530 [M + H]⁺ |
| 246A | 6-Chloro-3-[[(1R)-1-[6-methyl-2- (1-methylpyrazol-4-yl)-4-oxo- chromen-8-yl]ethyl]amino]-N- methylsulfonyl-pyridine-2- carboxamide | 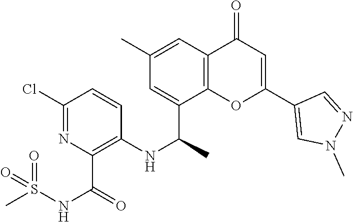 [SMILES: Cc1cc([C@@H](C)Nc2ccc(Cl)nc2C(=O)NS(C)(=O)=O)c2oc(-c3cnn(C)c3)cc(=O)c2c1] | 516 [M + H]⁺ |
| 247A | 6-Chloro-3-[[(1R)-1-[3,6- dimethyl-2-(1-methylimidazol-4- yl)-4-oxo-chromen-8- yl]ethyl]amino]-N- methylsulfonyl-pyridine-2- carboxamide | 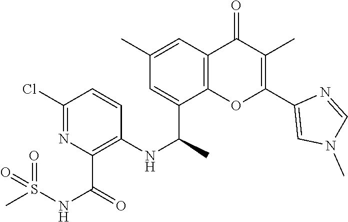 [SMILES: Cc1cc([C@@H](C)Nc2ccc(Cl)nc2C(=O)NS(C)(=O)=O)c2oc(-c3cn(C)cn3)c(C)c(=O)c2c1] | 530 [M + H]⁺ |
| 248A | 6-Chloro-3-[[(1R)-1-[2-(5-fluoro- 3-pyridyl)-3,6-dimethyl-4-oxo- chromen-8-yl]ethyl]amino]-N- methylsulfonyl-pyridine-2- carboxamide | 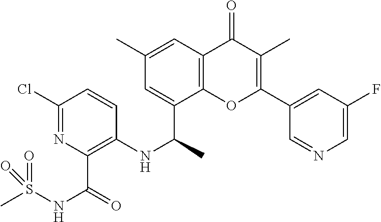 [SMILES: Cc1cc([C@@H](C)Nc2ccc(Cl)nc2C(=O)NS(C)(=O)=O)c2oc(-c3cncc(F)c3)c(C)c(=O)c2c1] | 545 [M + H]⁺ |
| 249A | 6-Chloro-3-[[(1R)-1-[3,6- dimethyl-2-(1-methyltriazol-4- yl)-4-oxo-chromen-8- yl]ethyl]amino]-N- methylsulfonyl-pyridine-2- carboxamide | 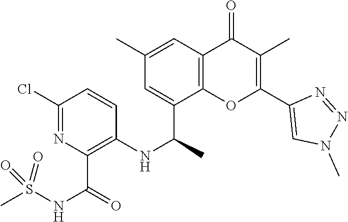 [SMILES: Cc1cc([C@@H](C)Nc2ccc(Cl)nc2C(=O)NS(C)(=O)=O)c2oc(-c3cn(C)nn3)c(C)c(=O)c2c1] | 531 [M + H]⁺ |
| 250A | 6-Chloro-3-[[(1R)-1-[2-(3-cyano- 5-fluoro-phenyl)-3,6-dimethyl-4- oxo-chromen-8-yl]ethyl]amino]- N-methylsulfonyl-pyridine-2- carboxamide | 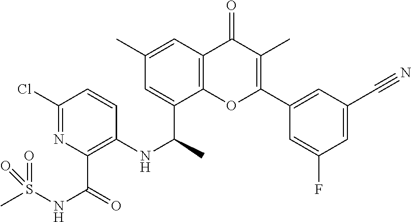 [SMILES: Cc1cc([C@@H](C)Nc2ccc(Cl)nc2C(=O)NS(C)(=O)=O)c2oc(-c3cc(F)cc(C#N)c3)c(C)c(=O)c2c1] | 569 [M + H]⁺ |
| 251A | 6-Chloro-3-[[(1R)-1-[2-(5-cyano- 3-pyridyl)-3,6-dimethyl-4-oxo- chromen-8-yl]ethyl]amino]-N- methylsulfonyl-pyridine-2- carboxamide | 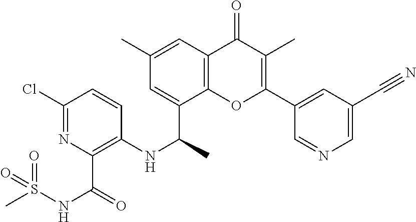 [SMILES: Cc1cc([C@@H](C)Nc2ccc(Cl)nc2C(=O)NS(C)(=O)=O)c2oc(-c3cncc(C#N)c3)c(C)c(=O)c2c1] | 552 [M + H]⁺ |
| 97C | 6-Chloro-3-[[(1R)-1-[3,6- dimethyl-2-[1-(oxetan-3- yl)pyrazol-4-yl]-4-oxo-chromen- 8-yl]ethyl]amino]-N- methylsulfonyl-pyridine-2- carboxamide | 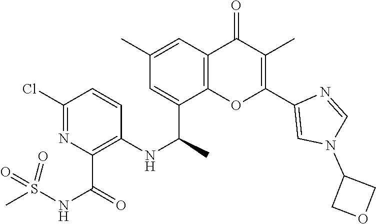 [SMILES: Cc1cc([C@@H](C)Nc2ccc(Cl)nc2C(=O)NS(C)(=O)=O)c2oc(-c3cn(C4COC4)cn3)c(C)c(=O)c2c1] | 572 [M + H]⁺ |
| 98C | 6-Chloro-3-[[(1R)-1-[2-(2-cyano- 5-fluoro-phenyl)-3,6-dimethyl-4- oxo-chromen-8-yl]ethyl]amino]- N-methylsulfonyl-pyridine-2- carboxamide | 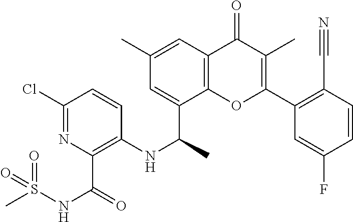 [SMILES: Cc1cc([C@@H](C)Nc2ccc(Cl)nc2C(=O)NS(C)(=O)=O)c2oc(-c3cc(F)ccc3C#N)c(C)c(=O)c2c1] | 569 [M + H]⁺ |
| 99C | 6-Chloro-3-[[(1R)-1-[2-(2-cyano- 3-fluoro-phenyl)-3,6-dimethyl-4- oxo-chromen-8-yl]ethyl]amino]- N-methylsulfonyl-pyridine-2- carboxamide | 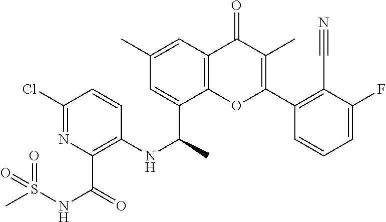 [SMILES: Cc1cc([C@@H](C)Nc2ccc(Cl)nc2C(=O)NS(C)(=O)=O)c2oc(-c3cccc(F)c3C#N)c(C)c(=O)c2c1] | 569 [M + H]⁺ |
| 100C | 6-Chloro-3-[[(1R)-1-[2-(2- cyanophenyl)-3,6-dimethyl-4- oxo-chromen-8-yl]ethyl]amino]- N-methylsulfonyl-pyridine-2- carboxamide | 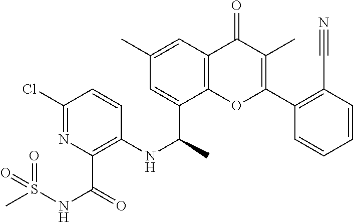 [SMILES: Cc1cc([C@@H](C)Nc2ccc(Cl)nc2C(=O)NS(C)(=O)=O)c2oc(-c3ccccc3C#N)c(C)c(=O)c2c1] | 551 [M + H]⁺ |
| 101C | 6-Chloro-3-[[(1R)-1-[2-(1,5- dimethylpyrazol-4-yl)-3,6- dimethyl-4-oxo-chromen-8- yl]ethyl]amino]-N- methylsulfonyl-pyridine-2- carboxamide | 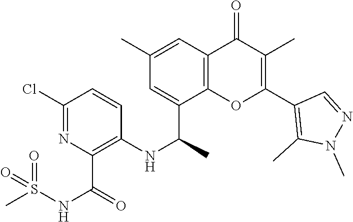 [SMILES: Cc1cc([C@@H](C)Nc2ccc(Cl)nc2C(=O)NS(C)(=O)=O)c2oc(-c3cnn(C)c3C)c(C)c(=O)c2c1] | 544 [M + H]⁺ |
| 102C | 6-Chloro-3-[[(1R)-1-[2-(1,3- dimethylpyrazol-4-yl)-3,6- dimethyl-4-oxo-chromen-8- ylethyl]amino]-N-methylsulfonyl- pyridine-2-carboxamide | 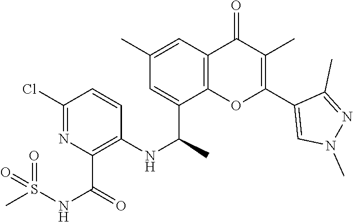 [SMILES: Cc1cc([C@@H](C)Nc2ccc(Cl)nc2C(=O)NS(C)(=O)=O)c2oc(-c3cn(C)nc3C)c(C)c(=O)c2c1] | 544 [M + H]⁺ |
| 103C | 6-Chloro-3-[[(1R)-1-[2-(5-cyano- 2-fluoro-phenyl)-3,6-dimethyl-4- oxo-chromen-8-yl]ethyl]amino]- N-methylsulfonyl-pyridine-2- carboxamide | 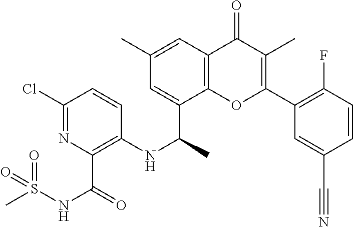 [SMILES: Cc1cc([C@@H](C)Nc2ccc(Cl)nc2C(=O)NS(C)(=O)=O)c2oc(-c3cc(C#N)ccc3F)c(C)c(=O)c2c1] | 569 [M + H]⁺ |
| 104C | 6-Chloro-3-[[(1R)-1-[3,6- dimethyl-2-(1-methylindazol-5- yl)-4-oxo-chromen-8- yl]ethyl]amino]-N- methylsulfonyl-pyridine-2- carboxamide | 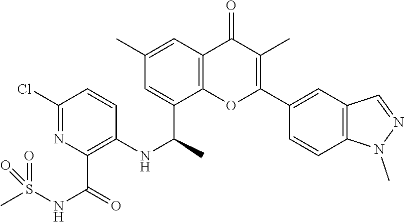 [SMILES: Cc1cc([C@@H](C)Nc2ccc(Cl)nc2C(=O)NS(C)(=O)=O)c2oc(-c3ccc4c(cnn4C)c3)c(C)c(=O)c2c1] | 580 [M + H]⁺ |
| 105C | 6-[[(1R)-1-[3,6-Dimethyl-2-(1- methylpyrazol-4-yl)-4-oxo- chromen-8-ylethyl]amino]-2,3- difluoro-N-methylsulfonyl- benzamide | 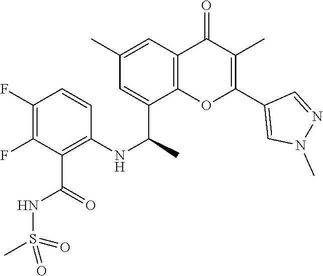 [SMILES: Cc1cc([C@@H](C)Nc2ccc(F)c(F)c2C(=O)NS(C)(=O)=O)c2oc(-c3cnn(C)c3)c(C)c(=O)c2c1] | 531 [M + H]⁺ |
| 106C | 6-Chloro-3-[[(1R)-1-[3,6- dimethyl-4-oxo-2-(3- pyridyl)chromen-8- yl]ethyl]amino]-N- methylsulfonyl-pyridine-2- carboxamide | 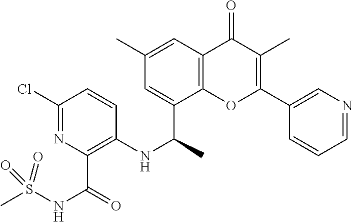 [SMILES: Cc1cc([C@@H](C)Nc2ccc(Cl)nc2C(=O)NS(C)(=O)=O)c2oc(-c3cccnc3)c(C)c(=O)c2c1] | 527 [M + H]⁺ |
| 107C | 6-Chloro-3-[[(1R)-1-[2-(2,4- difluorophenyl)-3,6-dimethyl-4- oxo-chromen-8-yl]ethyl]amino]- N-methylsulfonyl-pyridine-2- carboxamide | 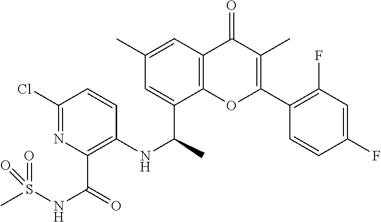 [SMILES: Cc1cc([C@@H](C)Nc2ccc(Cl)nc2C(=O)NS(C)(=O)=O)c2oc(-c3ccc(F)cc3F)c(C)c(=O)c2c1] | 562 [M + H]⁺ |
| 108C | 6-Chloro-3-[[(1R)-1-[2-(2,3- difluorophenyl)-3,6-dimethyl-4- oxo-chromen-8-yl]ethyl]amino]- N-methylsulfonyl-pyridine-2- carboxamide | 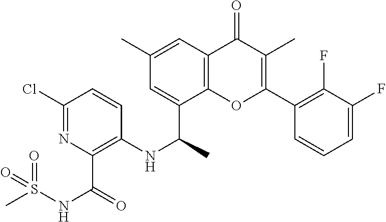 [SMILES: Cc1cc([C@@H](C)Nc2ccc(Cl)nc2C(=O)NS(C)(=O)=O)c2oc(-c3cccc(F)c3F)c(C)c(=O)c2c1] | 562 [M + H]⁺ |
| 109C | 6-Chloro-3-[[(1R)-1-[3,6- dimethyl-2-(2- methylpyrazolo[3,4-b]pyridin-5- yl)-4-oxo-chromen-8- ylethyl]amino]-N-methylsulfonyl- pyridine-2-carboxamide | 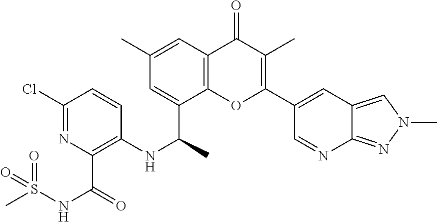 | 581 [M + H]⁺ |
| 110C | 6-Chloro-3-[[(1R)-1-[2-(6,7- dihydro-4H-pyrazolo[5,1- c][1,4]oxazin-3-yl)-3,6-dimethyl- 4-oxo-chromen-8-ylethyl]amino]- N-methylsulfonyl-pyridine-2- carboxamide | 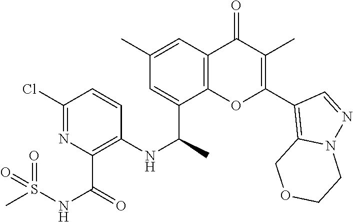 [SMILES: Cc1cc([C@@H](C)Nc2ccc(Cl)nc2C(=O)NS(C)(=O)=O)c2oc(-c3cnn4c3COCC4)c(C)c(=O)c2c1] | 572 [M + H]⁺ |
| 111C | 6-Chloro-3-[[(1R)-1-(3,6- dimethyl-4-oxo-2-pyrazolo[1,5- a]pyrimidin-3-yl-chromen-8- yl)ethyl]amino]-N- methylsulfonyl-pyridine-2- carboxamide | 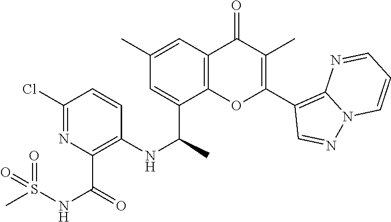 [SMILES: Cc1cc([C@@H](C)Nc2ccc(Cl)nc2C(=O)NS(C)(=O)=O)c2oc(-c3cnn4cccnc34)c(C)c(=O)c2c1] | 567 [M + H]⁺ |
| 112C | 6-Chloro-3-[[(1R)-1-[3,6- dimethyl-4-oxo-2-[3- (trifluoromethyl)-1- bicyclo[1.1.1]pentanyl]chromen- 8-yl]ethyl]amino]-N- methylsulfonyl-pyridine-2- carboxamide | 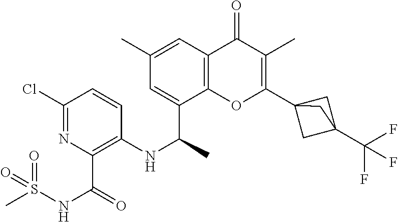 [SMILES: Cc1cc([C@@H](C)Nc2ccc(Cl)nc2C(=O)NS(C)(=O)=O)c2oc(C34CC(C(F)(F)F)(C3)C4)c(C)c(=O)c2c1] | 584 [M + H]⁺ |
| 113C | 6-Chloro-3-[1-[6-fluoro-3- methyl-2-(1-methylpyrazol-4-yl)- 4-oxo-chromen-8-yl]ethylamino]- N-methylsulfonyl-pyridine-2- carboxamide, Isomer 1 | 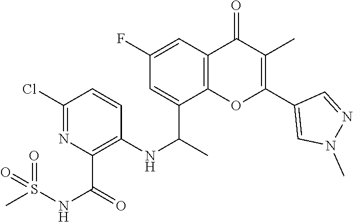 [SMILES: Cc1c(-c2cnn(C)c2)oc2c(C(C)Nc3ccc(Cl)nc3C(=O)NS(C)(=O)=O)cc(F)cc2c1=O] | 534 [M + H]⁺ |
| 114C | 6-Chloro-3-[1-[2-(3,4- difluorophenyl)-3-methyl-4-oxo- 6-(trifluoromethyl)chromen-8- yl]ethylamino]-N-methylsulfonyl- pyridine-2-carboxamide, Isomer 2 | 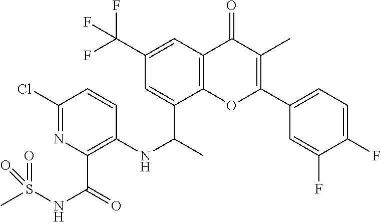 [SMILES: Cc1c(-c2ccc(F)c(F)c2)oc2c(C(C)Nc3ccc(Cl)nc3C(=O)NS(C)(=O)=O)cc(C(F)(F)F)cc2c1=O] | 616 [M + H]⁺ |
| 115C | 6-Chloro-3-[1-[2-(2,4- difluorophenyl)-3-methyl-4-oxo- 6-(trifluoromethyl)chromen-8- yl]ethylamino]-N-methylsulfonyl- pyridine-2-carboxamide, Isomer 2 | 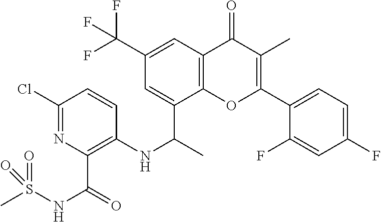 [SMILES: Cc1c(-c2ccc(F)cc2F)oc2c(C(C)Nc3ccc(Cl)nc3C(=O)NS(C)(=O)=O)cc(C(F)(F)F)cc2c1=O] | 616 [M + H]⁺ |

### Example 252A: 6-Chloro-3-[[(1R)-1-[3,6-dimethyl-4-oxo-2-(2-pyridyl)chromen-8-yl]ethyl]amino]-N-methylsulfonyl-pyridine-2-carboxamide

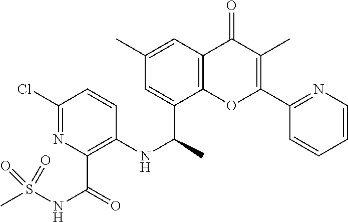 [SMILES: Cc1cc([C@@H](C)Nc2ccc(Cl)nc2C(=O)NS(C)(=O)=O)c2oc(-c3ccccn3)c(C)c(=O)c2c1]

6-Chloro-3-[[(1R)-1-[3,6-dimethyl-4-oxo-2-(2-pyridyl)chromen-8-yl]ethyl]amino]pyridine-2-carboxylic acid (0.10 g, 0.22 mmol), methanesulfonamide (0.03 g, 0.29 mmol), and N,N′-dicyclohexylcarbodiimide (0.06 g, 0.29 mmol) were combined in 6 mL of DCM and the reaction allowed to stir at 25° C. When the reaction was complete, reaction was directly loaded and purified by reversed phase chromatography eluted with 10% to 100% ACN (with 0.1% formic acid) in water (with 0.1% formic acid) to give the title compound (62.1 mg, 53%). MS ES+ m/z 527 [M+H]⁺.

The following compounds in Table 79 were made in a similar way as described for 6-chloro-3-[[(1R)-1-[3,6-dimethyl-4-oxo-2-(2-pyridyl)chromen-8-yl]ethyl]amino]-N-methylsulfonyl-pyridine-2-carboxamide. Various methods were used to purify the compounds, which would be apparent to one skilled in the art.

TABLE 79

| Example | | | MS ES+ |
|---|---|---|---|
| # | Chemical Name | Structure | m/z |
| 116C¹ | 6-Chloro-3-[[(1R)-1-[2-[6-(1- cyanocyclopropyl)-3- pyridyl]-3,6-dimethyl-4-oxo- chromen-8-yl]ethyl]amino]- N-methylsulfonyl-pyridine- 2-carboxamide | 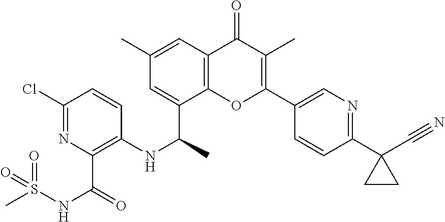 [SMILES: Cc1cc([C@@H](C)Nc2ccc(Cl)nc2C(=O)NS(C)(=O)=O)c2oc(-c3ccc(C4(C#N)CC4)nc3)c(C)c(=O)c2c1] | 592 [M + H]⁺ |
| 117C¹ | 3-[[(1R)-1-[2-(1,3- Benzodioxol-5-yl)-3,6- dimethyl-4-oxo-chromen-8- yl]ethyl]amino]-6-chloro-N- methylsulfonyl-pyridine-2- carboxamide | 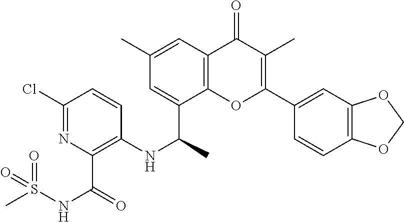 [SMILES: Cc1cc([C@@H](C)Nc2ccc(Cl)nc2C(=O)NS(C)(=O)=O)c2oc(-c3ccc4c(c3)OCO4)c(C)c(=O)c2c1] | 570 [M + H]⁺ |
| 118C¹ | 6-Chloro-3-[[(1R)-1-[3,6- dimethyl-2-(1- methylpyrazol-4-yl)-4-oxo- chromen-8-yl]ethyl]amino]- N-(2- pyridylsulfonyl)pyridine-2- carboxamide | 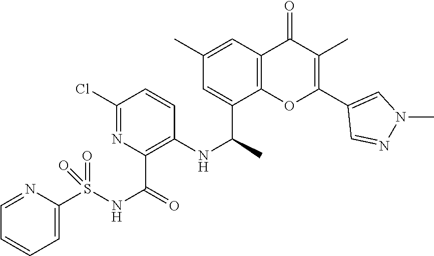 [SMILES: Cc1cc([C@@H](C)Nc2ccc(Cl)nc2C(=O)NS(=O)(=O)c2ccccn2)c2oc(-c3cnn(C)c3)c(C)c(=O)c2c1] | 593 [M + H]⁺ |
| 119C¹ | N-(Benzenesulfonyl)-6- chloro-3-[[(1R)-1-[3,6- dimethyl-2-(1- methylpyrazol-4-yl)-4-oxo- chromen-8- yl]ethyl]amino]pyridine-2- carboxamide | 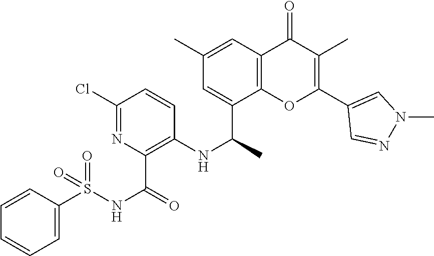 [SMILES: Cc1cc([C@@H](C)Nc2ccc(Cl)nc2C(=O)NS(=O)(=O)c2ccccc2)c2oc(-c3cnn(C)c3)c(C)c(=O)c2c1] | 592 [M + H]⁺ |
| 120C¹ | 3-[[(1R)-1-[3,6-Dimethyl-2- (1-methylpyrazol-4-yl)-4- oxo-chromen-8- yl]ethyl]amino]-6-fluoro-N- methylsulfonyl-pyridine-2- carboxamide | 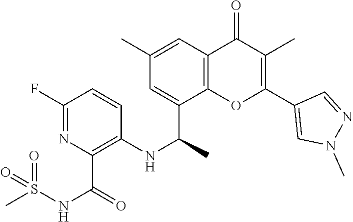 [SMILES: Cc1cc([C@@H](C)Nc2ccc(F)nc2C(=O)NS(C)(=O)=O)c2oc(-c3cnn(C)c3)c(C)c(=O)c2c1] | 514 [M + H]⁺ |
| 121C¹ | 6-Chloro-3-[[(1R)-1-[2-(3- cyano-2-fluoro-phenyl)-3,6- dimethyl-4-oxo-chromen-8- yl]ethyl]amino]pyridine-2- carboxamide | 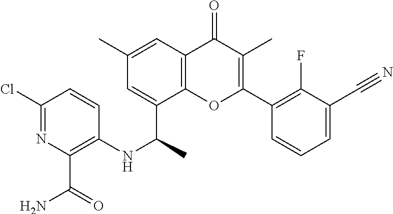 [SMILES: Cc1cc([C@@H](C)Nc2ccc(Cl)nc2C(N)=O)c2oc(-c3cccc(C#N)c3F)c(C)c(=O)c2c1] | 491 [M + H]⁺ |
| 122C¹ | 6-Chloro-3-[[(1R)-1-[3,6- dimethyl-4-oxo-2-(1H- pyrazol-4-yl)chromen-8- yl]ethyl]amino]-N- methylsulfonyl-pyridine-2- carboxamide | 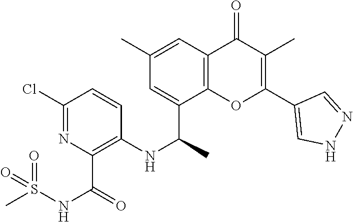 [SMILES: Cc1cc([C@@H](C)Nc2ccc(Cl)nc2C(=O)NS(C)(=O)=O)c2oc(-c3cn[nH]c3)c(C)c(=O)c2c1] | 516 [M + H]⁺ |
| 123C¹ | 6-Chloro-3-[[(1R)-1-[3,6- dimethyl-2-(1- methylindazol-3-yl)-4-oxo- chromen-8-yl]ethyl]amino]- N-methylsulfonyl-pyridine- 2-carboxamide | 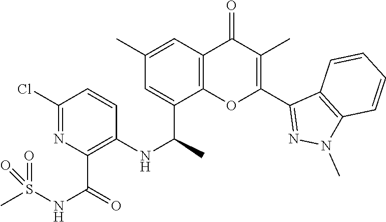 [SMILES: Cc1cc([C@@H](C)Nc2ccc(Cl)nc2C(=O)NS(C)(=O)=O)c2oc(-c3nn(C)c4ccccc34)c(C)c(=O)c2c1] | 580 [M + H]⁺ |
| 124C² | 6-Chloro-3-[[(1R)-1-[3,6- dimethyl-2-(2- methylpyrazolo[4,3- b]pyridin-5-yl)-4-oxo- chromen-8-yl]ethyl]amino]- N-methylsulfonyl-pyridine- 2-carboxamide | 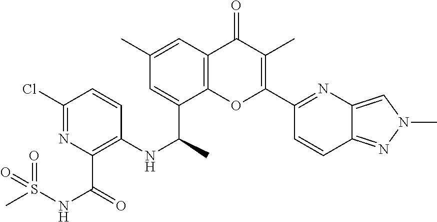 | 581 [M + H]⁺ |
| 125C¹ | 6-Chloro-3-[[(1R)-1-[2-(2,6- difluorophenyl)-3,6- dimethyl-4-oxo-chromen-8- yl]ethyl]amino]-N- methylsulfonyl-pyridine-2- carboxamide | 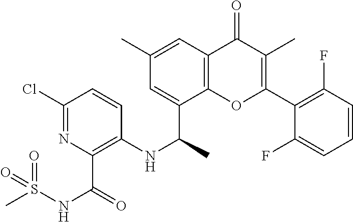 [SMILES: Cc1cc([C@@H](C)Nc2ccc(Cl)nc2C(=O)NS(C)(=O)=O)c2oc(-c3c(F)cccc3F)c(C)c(=O)c2c1] | 562 [M + H]⁺ |
| 126C¹ | 3-[[(1R)-1-[2-(1,3- Benzoxazol-4-yl)-3,6- dimethyl-4-oxo-chromen-8- yl]ethyl]amino]-6-chloro-N- methylsulfonyl-pyridine-2- carboxamide | 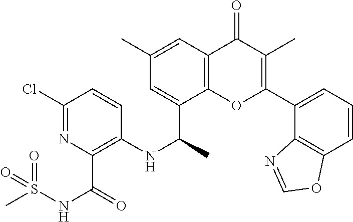 [SMILES: Cc1cc([C@@H](C)Nc2ccc(Cl)nc2C(=O)NS(C)(=O)=O)c2oc(-c3cccc4ocnc34)c(C)c(=O)c2c1] | 567 [M + H]⁺ |
| 127C¹ | 2-[1-[6-Fluoro-2-(1H-indol- 2-yl)-4-oxo-chromen-8- yl]ethylamino]-N- methylsulfonyl-benzamide, Isomer 1 | 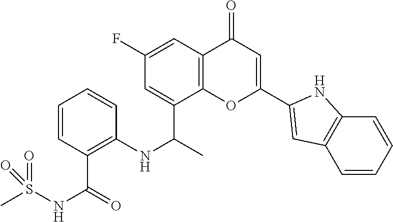 [SMILES: CC(Nc1ccccc1C(=O)NS(C)(=O)=O)c1cc(F)cc2c(=O)cc(-c3cc4ccccc4[nH]3)oc12] | 520 [M + H]⁺ |

¹4-Dimethylaminopyridine used in reaction.
²EDC and DMAP used for this reaction.

### Example 101A: 8-[(1R)-1-[[2-(5-Amino-1,3,4-thiadiazol-2-yl)-3-pyridyl]amino]ethyl]-3,6-dimethyl-2-phenyl-chromen-4-one

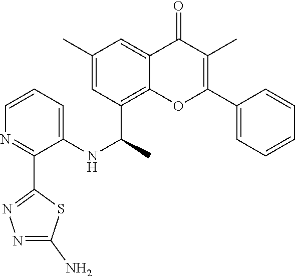 [SMILES: Cc1cc([C@@H](C)Nc2cccnc2-c2nnc(N)s2)c2oc(-c3ccccc3)c(C)c(=O)c2c1]

Combined 3-[[(1R)-1-(3,6-dimethyl-4-oxo-2-phenyl-chromen-8-yl)ethyl]amino]pyridine-2-carbonitrile (0.095 g, 0.24 mmol), thiosemicarbazide (0.033 g, 0.36 mmol) and TFA (2 mL). The reaction was heated at 80° C. for 4 h, concentrated, and diluted with DCM and saturated aqueous NaHCO₃. The aqueous layer was extracted 3× with IPA:CHCl₃ (1:3). The combined organic extracts were dried over MgSO₄, filtered, and concentrated. The residue was purified by reverse phase chromatography eluted with a gradient of 0% to 100% ACN in H₂O+0.1% formic acid. Fractions containing the desired product were combined, washed with saturated aqueous sodium chloride, and extracted with IPA:CHCl₃ (1:3). The combined extracts were dried over MgSO₄, filtered, and concentrated. The material was recrystallized from a mixture of DCM and hexanes to give the title compound (0.025 g, 22%) as a white solid. MS ES+ m/z 470 [M+H]⁺.

### Example 102A: 3,6-Dimethyl-8-[(1R)-1-[2-(1-methyltriazol-4-yl)anilino]ethyl]-2-phenyl-chromen-4-one

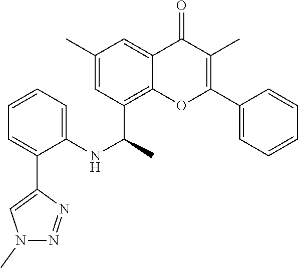 [SMILES: Cc1cc([C@@H](C)Nc2ccccc2-c2cn(C)nn2)c2oc(-c3ccccc3)c(C)c(=O)c2c1]

To a mixture of 8-[(1R)-1-(2-ethynylanilino)ethyl]-3,6-dimethyl-2-phenyl-chromen-4-one (0.100 g, 0.254 mmol) in tert-butanol (2 mL) and water (2 mL) was added sodium azide (0.025 g, 0.381 mmol), iodomethane (0.024 mL, 0.381 mmol), and cuprous iodide (0.0097 g, 0.051 mmol). The reaction was heated at 80° C. overnight, then diluted with EtOAc and saturated aqueous NaHCO₃. The aqueous layer was extracted 2× with EtOAc. The combined organic extracts were dried over Na₂SO₄, filtered, and concentrated. The residue was purified by silica gel chromatography eluted with 0% to 100% EtOAc in hexanes. Fractions containing the desired product were combined and concentrated. The material was recrystallized from a mixture of DCM and hexanes to give the title compound (0.0042 g, 3.7%). MS ES+ m/z 451 [M+H]⁺.

The following compound in Table 80 was made in a similar way as described for 3,6-dimethyl-8-[(1R)-1-[2-(1-methyltriazol-4-yl)anilino]ethyl]-2-phenyl-chromen-4-one.

TABLE 80

| Example | | | MS ES+ |
|---|---|---|---|
| # | Chemical Name | Structure | m/z |
| 103A | 8-[(1R)-1-[2-(1- Benzyltriazol-4- yl)anilino]ethyl]- 3,6-dimethyl-2- phenyl-chromen- 4-one | 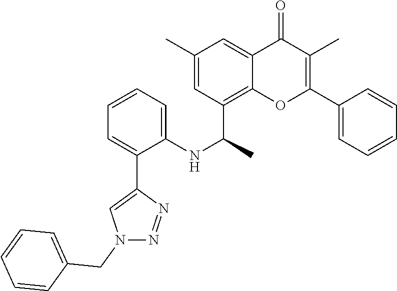 [SMILES: Cc1cc([C@@H](C)Nc2ccccc2-c2cn(Cc3ccccc3)nn2)c2oc(-c3ccccc3)c(C)c(=O)c2c1] | 527 [M + H]⁺ |

### Example 253A: 6-Chloro-3-[[(1R)-1-(3,6-dimethyl-4-oxo-2-phenyl-chromen-8-yl)ethyl]amino]pyridine-2-sulfonamide

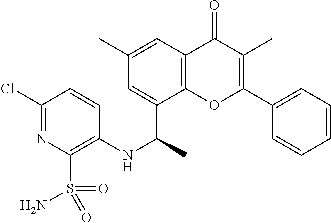 [SMILES: Cc1cc([C@@H](C)Nc2ccc(Cl)nc2S(N)(=O)=O)c2oc(-c3ccccc3)c(C)c(=O)c2c1]

1,3-Dichloro-5,5-dimethylhydantoin (0.17 g, 0.85 mmol), ACN (4 mL), water (0.3 mL), and acetic acid (0.35 mL) were allowed to stir at 0° C. for 5 min and then treated with an ACN solution of (8-[(1R)-1-[(2-benzylsulfanyl-6-chloro-3-pyridyl)amino]ethyl]-3,6-dimethyl-2-phenyl-chromen-4-one (0.30 g, 0.57 mmol) and stirred at 0° C. for 2 h. Ammonia (0.1 mg, 5.69 mmol) was added and stirring continued for 30 min. Reaction was diluted with 10 mL of dichloromethane and 10 mL of water. The reaction was extracted with dichloromethane (3×5 mL, organics collected, dried over MgSO₄, concentrated, and the residue purified by reverse phase chromatography eluted with 10% to 100% ACN in aqueous 0.5 μM NH₄HCO₃ containing 5% MeOH to give the product (0.07 g, 25%). MS ES+ m/z 484 [M+H]⁺.

The following compound in Table 81 was made in a similar way as described for 6-chloro-3-[[(1R)-1-(3,6-dimethyl-4-oxo-2-phenyl-chromen-8-yl)ethyl]amino]pyridine-2-sulfonamide.

TABLE 81

| Example | | | MS ES+ |
|---|---|---|---|
| # | Chemical Name | Structure | m/z |
| 254A | 6-Chloro-3-[[(1R)-1- [3,6-dimethyl-2-(2- methylindazol-5-yl)-4- oxo-chromen-8- yl]ethyl]amino]pyridine- 2-sulfonamide | 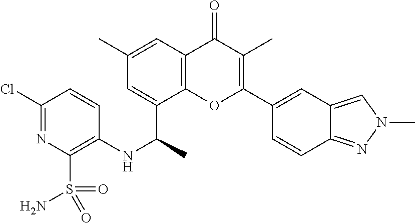 [SMILES: Cc1cc([C@@H](C)Nc2ccc(Cl)nc2S(N)(=O)=O)c2oc(-c3ccc4nn(C)cc4c3)c(C)c(=O)c2c1] | 538 [M + H]⁺ |

### Example 255A: N-[[6-Chloro-3-[[(1R)-1-(3,6-dimethyl-4-oxo-2-phenyl-chromen-8-yl)ethyl]amino]-2-pyridyl]sulfonyl]acetamide

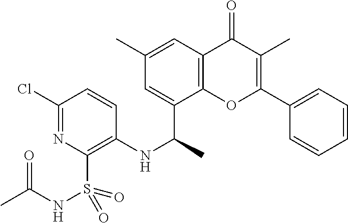 [SMILES: CC(=O)NS(=O)(=O)c1nc(Cl)ccc1N[C@H](C)c1cc(C)cc2c(=O)c(C)c(-c3ccccc3)oc12]

Combined 6-chloro-3-[[(1R)-1-(3,6-dimethyl-4-oxo-2-phenyl-chromen-8-yl)ethyl]amino]pyridine-2-sulfonamide (20 mg, 0.04 mmol), acetic acid (7.4 mg, 0.12 mmol), DCC (34 mg, 0.17 mmol), and DMAP (30 mg, 0.25 mmol) in DCM (1 mL) and allowed to stir at 25° C. overnight. Reaction was diluted with 1 mL of MeOH and purified by reversed phase chromatography eluted with 10% to 100% ACN (with 0.1% formic acid) in water (with 0.1% formic acid) to give the title compound (5 mg, 20%). MS ES+ m/z 526 [M+H]⁺.

The following compound in Table 82 was made in a similar way as described for N-[[6-chloro-3-[[(1R)-1-(3,6-dimethyl-4-oxo-2-phenyl-chromen-8-yl)ethyl]amino]-2-pyridyl]sulfonyl]acetamide. Various methods were used to purify the compounds, which would be apparent to one skilled in the art.

TABLE 82

| Example | | | MS ES+ |
|---|---|---|---|
| # | Chemical Name | Structure | m/z |
| 128C¹ | 6-Chloro-3-[[(1R)-1-[3,6- dimethyl-2-(1- methylpyrazol-4-yl)-4- oxo-chromen-8- yl]ethyl]amino]-N-(3- pyridylsulfonyl)pyridine- 2-carboxamide | 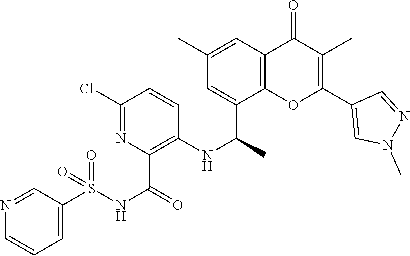 [SMILES: Cc1cc([C@@H](C)Nc2ccc(Cl)nc2C(=O)NS(=O)(=O)c2cccnc2)c2oc(-c3cnn(C)c3)c(C)c(=O)c2c1] | 593 [M + H]⁺ |

### Example 256A: Ethyl N-[2-[[(1R)-1-(3,6-dimethyl-4-oxo-2-phenyl-chromen-8-yl)ethyl]amino]phenyl]sulfonylcarbamate

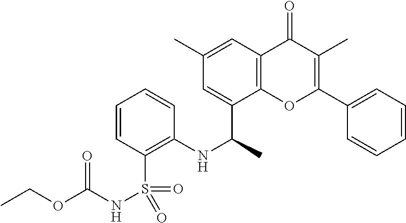 [SMILES: CCOC(=O)NS(=O)(=O)c1ccccc1N[C@H](C)c1cc(C)cc2c(=O)c(C)c(-c3ccccc3)oc12]

A solution of 2-[[(1R)-1-(3,6-dimethyl-4-oxo-2-phenyl-chromen-8-yl)ethyl]amino]benzenesulfonamide (30 mg, 0.07 mmol) in acetone (1 mL) was treated with potassium carbonate (28 mg, 0.20 mmol) and ethyl chloroformate (15 mg, 0.13 mmol) and stirred at 70° C. overnight. The reaction was allowed to cool to rt, concentrated, and the residue purified by silica gel chromatography eluted with 0% to 20% MeOH in DCM followed by purification by preparative TLC eluted with 5% MeOH in DCM to give the title compound (17.8 mg, 51%) as a white solid. MS ES+ m/z 521 [M+H]⁺.

### Example 257A: N-Cyano-2-[[(1R)-1-(3,6-dimethyl-4-oxo-2-phenyl-chromen-8-yl)ethyl]amino]benzenesulfonamide

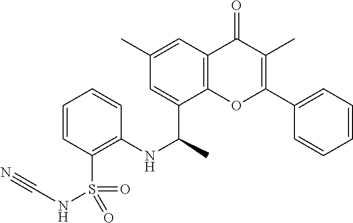 [SMILES: Cc1cc([C@@H](C)Nc2ccccc2S(=O)(=O)NC#N)c2oc(-c3ccccc3)c(C)c(=O)c2c1]

A solution of 2-[[(1R)-1-(3,6-dimethyl-4-oxo-2-phenyl-chromen-8-yl)ethyl]amino]benzenesulfonamide (36 mg, 0.08 mmol) in 1 mL of ACN was treated with triethylamine (24 mg, 0.24 mmol) and cyanogen bromide (17 mg, 0.16 mmol). The resulting mixture was stirred at rt overnight. The reaction was concentrated and the crude material purified by preparative TLC eluted with 5% MeOH in DCM. The desired material was crystallized from a mixture of DCM/MTBE/hexanes to give the title compound (13.6 mg, 36%) as a pale yellow solid. MS ES+ m/z 474 [M+H]⁺.

### Example 129C: 2-[[(1R)-1-(3,6-Dimethyl-4-oxo-2-phenyl-chromen-8-yl)ethyl]amino]-N-methoxy-benzenesulfonamide

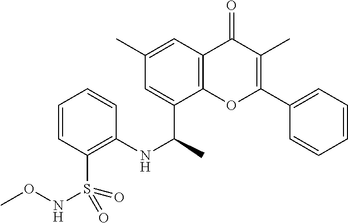 [SMILES: CONS(=O)(=O)c1ccccc1N[C@H](C)c1cc(C)cc2c(=O)c(C)c(-c3ccccc3)oc12]

Combined 2-fluoro-N-methoxy-benzenesulfonamide (0.05 g, 0.24 mmol), 8-[(1R)-1-aminoethyl]-3,6-dimethyl-2-phenyl-chromen-4-one (0.11 g, 0.37 mmol), and diisopropylethylamine (0.16 g, 1.2 mmol) in DMSO (1 mL). Stirred the reaction at 130° C. over the weekend. Reaction was purified twice by reversed phase chromatography eluted with 10% to 100% ACN (with 0.1% formic acid) in water (with 0.1% formic acid) to give the title compound (2.5 mg, 2%). ES/MS m/z 479 (M+H).

### Example 258A: 3-[[(1R)-1-(3,6-Dimethyl-4-oxo-2-phenyl-chromen-8-yl)ethyl]amino]-N-sulfamoyl-pyridine-2-carboxamide

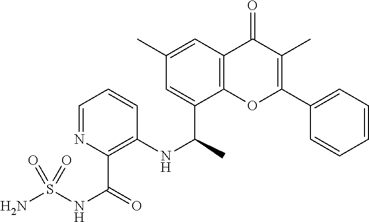 [SMILES: Cc1cc([C@@H](C)Nc2cccnc2C(=O)NS(N)(=O)=O)c2oc(-c3ccccc3)c(C)c(=O)c2c1]

A solution of tert-butyl N-[[3-[[(1R)-1-(3,6-dimethyl-4-oxo-2-phenyl-chromen-8-yl)ethyl]amino]pyridine-2-carbonyl]sulfamoyl]carbamate (0.12 g, 0.20 mmol) in DCM (1 mL) was treated with TFA (1 mL) and the reaction allowed to stir for 2 h. The reaction was concentrated and the residue purified by reversed phase C18 flash chromatography eluted with 0% to 100% ACN in water (with 0.1% formic acid). Fractions containing the product were pooled, washed with saturated aqueous sodium chloride, and extracted with IPA/CHCl₃ (1:3). The organics were combined, dried over MgSO₄, filtered, and concentrated. The residue was recrystallized from DCM/hexanes to give the title compound (9.5 mg, 10%) as a white solid. MS ES+ m/z 493 [M+H]⁺.

The following compound in Table 83 was made in a similar way as described for 3-[[(1R)-1-(3,6-dimethyl-4-oxo-2-phenyl-chromen-8-yl)ethyl]amino]-N-sulfamoyl-pyridine-2-carboxamide. Various methods were used to purify the compound, which would be apparent to one skilled in the art.

TABLE 83

| Example | | | MS ES+ |
|---|---|---|---|
| # | Chemical Name | Structure | m/z |
| 130C | 6-Chloro-3- [[(1R)-1-(3,6- dimethyl-4-oxo- 2-phenyl- chromen-8- yl)ethyl]amino]- N-sulfamoyl- pyridine-2- carboxamide | 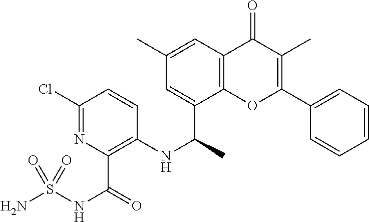 [SMILES: Cc1cc([C@@H](C)Nc2ccc(Cl)nc2C(=O)NS(N)(=O)=O)c2oc(-c3ccccc3)c(C)c(=O)c2c1] | 527 [M + H]⁺ |

The following compound in Table 84 was made in a similar way as described for 3-[[(1R)-1-(3,6-dimethyl-4-oxo-2-phenyl-chromen-8-yl)ethyl]amino]-N-sulfamoyl-pyridine-2-carboxamide.

TABLE 84

| Example | | | MS ES+ |
|---|---|---|---|
| # | Chemical Name | Structure | m/z |
| 259A | 8-[[(1R)-1-[3,6- Dimethyl-4-oxo- 2-(3- pyridyl)chromen- 8- yl]ethyl]amino]- 2H-isoquinolin- 1-one | 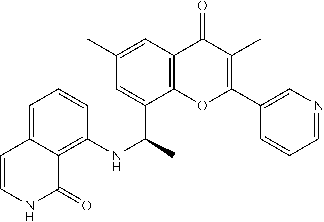 [SMILES: Cc1cc([C@@H](C)Nc2cccc3cc[nH]c(=O)c23)c2oc(-c3cccnc3)c(C)c(=O)c2c1] | 438 [M + H]⁺ |

### Example 260A: 6-Chloro-3-[[(1R)-1-[3,6-dimethyl-4-oxo-2-(3-pyridyl)chromen-8-yl]ethyl]amino]pyridine-2-sulfonamide

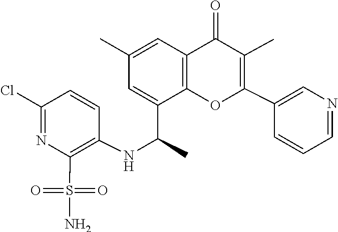 [SMILES: Cc1cc([C@@H](C)Nc2ccc(Cl)nc2S(N)(=O)=O)c2oc(-c3cccnc3)c(C)c(=O)c2c1]

A mixture of N-tert-butyl-6-chloro-3-[[(1R)-1-[3,6-dimethyl-4-oxo-2-(3-pyridyl)chromen-8-yl]ethyl]amino]pyridine-2-sulfonamide (0.13 g, 0.24 mmol) in 3 mL of DCM was treated with 3 mL of TFA and the reaction allowed to stir at 50° C. for 1 h. The reaction was allowed to cool and concentrated. The residue was basified to pH ˜8 with DIPEA and extracted with DCM (3×10 mL). The combined organics were washed with saturated aqueous sodium chloride (3×10 mL), collected, dried over Na₂SO₄, filtered, and concentrated. The residue was purified by reversed phase chromatography eluted with 25% to 60% ACN in water (with 0.1% formic acid) to give the title compound (23.1 mg, 20%) as a white solid. MS ES+ m/z 485 [M+H]⁺.

The following compounds in Table 85 were made in a similar way as described for 6-chloro-3-[[(1R)-1-[3,6-dimethyl-4-oxo-2-(3-pyridyl)chromen-8-yl]ethyl]amino]pyridine-2-sulfonamide. Various methods were used to purify the compounds, which would be apparent to one skilled in the art.

TABLE 85

| Example | | | MS ES+ |
|---|---|---|---|
| # | Chemical Name | Structure | m/z |
| 131C | 6-Chloro-3-[[(1R)-1-[2-(1,5- dimethyl-6-oxo-3-pyridyl)-6- methyl-4-oxo-chromen-8- yl]ethyl]amino]pyridine-2- sulfonamide | 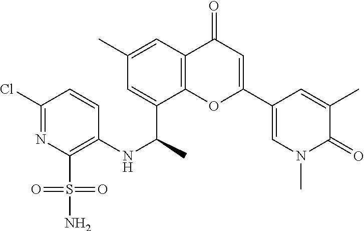 [SMILES: Cc1cc([C@@H](C)Nc2ccc(Cl)nc2S(N)(=O)=O)c2oc(-c3cc(C)c(=O)n(C)c3)cc(=O)c2c1] | 515 [M + H]⁺ |
| 132C | 6-Chloro-3-[(1R)-1-(3- isoxazol-4-yl-6-methyl-4- oxo-2-phenyl-chromen-8- yl)ethyl]amino]pyridine-2- sulfonamide | 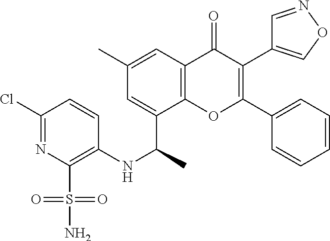 [SMILES: Cc1cc([C@@H](C)Nc2ccc(Cl)nc2S(N)(=O)=O)c2oc(-c3ccccc3)c(-c3cnoc3)c(=O)c2c1] | 537 [M + H]⁺ |
| 133C | 6-Chloro-3-[(1R)-1-[2-(1- isopropyl-6-oxo-3-pyridyl)- 3,6-dimethyl-4-oxo- chromen-8- yl]ethyl]amino]pyridine-2- sulfonamide | 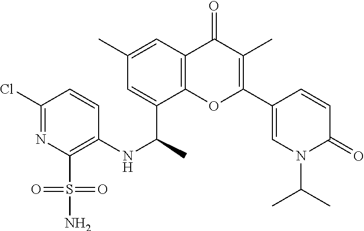 [SMILES: Cc1cc([C@@H](C)Nc2ccc(Cl)nc2S(N)(=O)=O)c2oc(-c3ccc(=O)n(C(C)C)c3)c(C)c(=O)c2c1] | 543 [M + H]⁺ |
| 134C | 6-Chloro-3-[[(1R)-1-[6- methyl-2-(1-methyl-6-oxo-3- pyridyl)-4-oxo-chromen-8- yl]ethyl]amino]pyridine-2- sulfonamide | 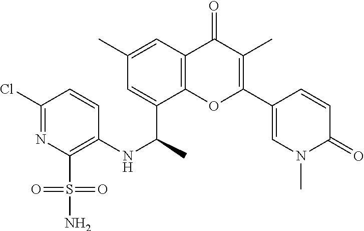 | 501 [M + H]⁺ |
| 135C | 6-Chloro-3-[(1R)-1-[6- methyl-2-(1-methylpyrazol- 4-y1)-4-oxo-chromen-8- yl]ethyl]amino]pyridine-2- sulfonamide | 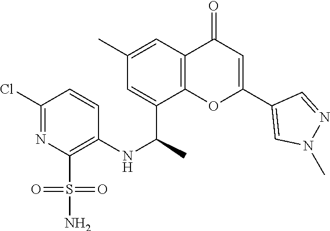 [SMILES: Cc1cc([C@@H](C)Nc2ccc(Cl)nc2S(N)(=O)=O)c2oc(-c3cnn(C)c3)cc(=O)c2c1] | 474 [M + H]⁺ |
| 136C | 6-Chloro-3-[(1R)-1-[3,6- dimethyl-2-(1-methyl-2-oxo- 4-pyridyl)-4-oxo-chromen-8- yl]ethyl]amino]pyridine-2- sulfonamide | 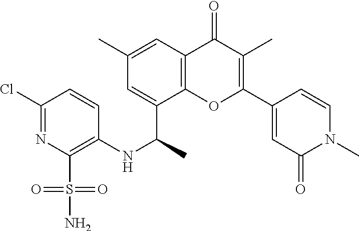 [SMILES: Cc1cc([C@@H](C)Nc2ccc(Cl)nc2S(N)(=O)=O)c2oc(-c3ccn(C)c(=O)c3)c(C)c(=O)c2c1] | 515 [M + H]⁺ |
| 137C | 6-Chloro-3-[(1R)-1-[3,6- dimethyl-2-(1-methyl-6-oxo- 3-pyridyl)-4-oxo-chromen-8- yl]ethyl]amino]pyridine-2- sulfonamide | 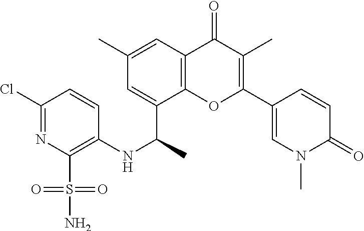 [SMILES: Cc1cc([C@@H](C)Nc2ccc(Cl)nc2S(N)(=O)=O)c2oc(-c3ccc(=O)n(C)c3)c(C)c(=O)c2c1] | 515 [M + H]⁺ |
| 138C | 6-Chloro-3-[[(1R)-1-[6- methyl-2-(2-methylindazol- 5-y1)-4-oxo-chromen-8- yl]ethyl]amino]pyridine-2- sulfonamide | 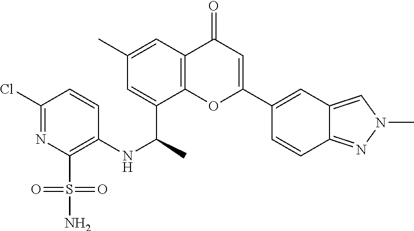 [SMILES: Cc1cc([C@@H](C)Nc2ccc(Cl)nc2S(N)(=O)=O)c2oc(-c3ccc4nn(C)cc4c3)cc(=O)c2c1] | 524 [M + H]⁺ |
| 139C | 6-Chloro-3-[[(1R)-1-[3,6- dimethyl-2-(1- methylpyrazol-4-yl)-4-oxo- chromen-8- yl]ethyl]amino]pyridine-2- sulfonamide | 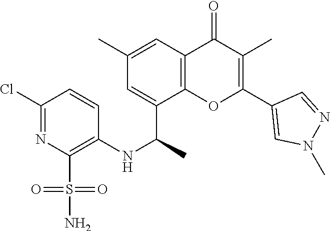 [SMILES: Cc1cc([C@@H](C)Nc2ccc(Cl)nc2S(N)(=O)=O)c2oc(-c3cnn(C)c3)c(C)c(=O)c2c1] | 488 [M + H]⁺ |
| 140C | 6-Chloro-3-[[(1R)-1-[3,6- dimethyl-2-(2- methylindazol-5-yl)-4-oxo- chromen-8- yl]ethyl]amino]pyridine-2- sulfonamide | 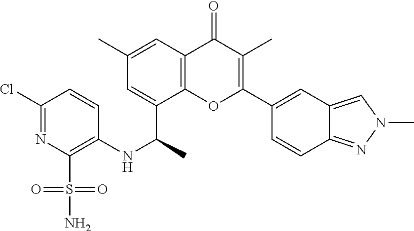 [SMILES: Cc1cc([C@@H](C)Nc2ccc(Cl)nc2S(N)(=O)=O)c2oc(-c3ccc4nn(C)cc4c3)c(C)c(=O)c2c1] | 538 [M + H]⁺ |
| 141C | 3-[[(1R)-1-[3,6-Dimethyl-2- (1-methylpyrazol-4-y1)-4- oxo-chromen-8- yl]ethyl]amino]pyridine-2- sulfonamide | 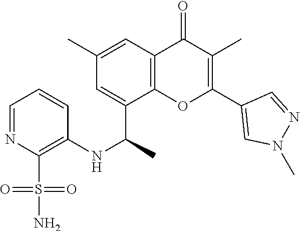 [SMILES: Cc1cc([C@@H](C)Nc2cccnc2S(N)(=O)=O)c2oc(-c3cnn(C)c3)c(C)c(=O)c2c1] | 454 [M + H]⁺ |
| 142C | 6-Chloro-3-[(1R)-1-[3,6- dimethyl-4-oxo-2-(2- pyridyl)chromen-8- yl]ethyl]amino]pyridine-2- sulfonamide | 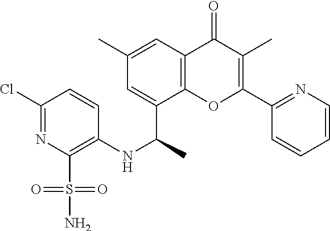 [SMILES: Cc1cc([C@@H](C)Nc2ccc(Cl)nc2S(N)(=O)=O)c2oc(-c3ccccn3)c(C)c(=O)c2c1] | 485 [M + H]⁺ |
| 143C | 6-Chloro-3-[(1R)-1-[2-[1- (difluoromethyl)pyrazol-4- yl]-3,6-dimethyl-4-oxo- chromen-8- yl]ethyl]amino]pyridine-2- sulfonamide | 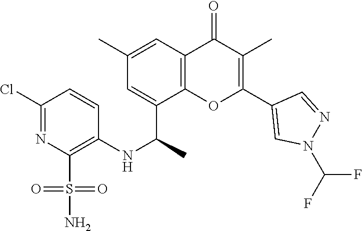 [SMILES: Cc1cc([C@@H](C)Nc2ccc(Cl)nc2S(N)(=O)=O)c2oc(-c3cnn(C(F)F)c3)c(C)c(=O)c2c1] | 524 [M + H]⁺ |
| 144C | 6-Chloro-3-[[(1R)-1-[3,6- dimethyl-4-oxo-2-[3- (trifluoromethyl)-1- bicyclo[1.1.1 ]pentanyl] chromen-8- yl]ethyl]amino]pyridine-2- sulfonamide | 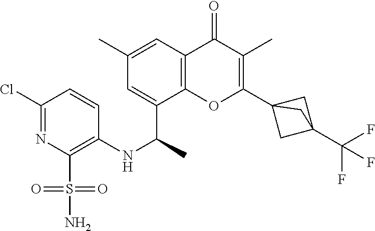 [SMILES: Cc1cc([C@@H](C)Nc2ccc(Cl)nc2S(N)(=O)=O)c2oc(C34CC(C(F)(F)F)(C3)C4)c(C)c(=O)c2c1] | 542 [M + H]⁺ |
| 145C | 6-Chloro-3-[(1R)-1-[2-(4,4- difluorocyclohexyl)-3,6- dimethyl-4-oxo-chromen-8- yl]ethyl]amino]pyridine-2- sulfonamide | 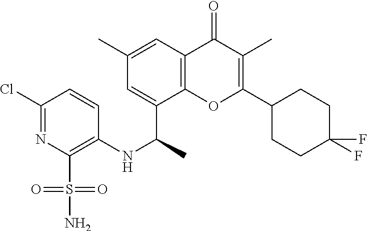 [SMILES: Cc1cc([C@@H](C)Nc2ccc(Cl)nc2S(N)(=O)=O)c2oc(C3CCC(F)(F)CC3)c(C)c(=O)c2c1] | 526 [M + H]⁺ |
| 237C | 6-Chloro-3-[[(1R)-1-[3,6- dimethyl-4-oxo-2-(2-oxo- 1H-pyridin-4-y1)chromen-8- yl]ethyl]amino]pyridine-2- sulfonamide | 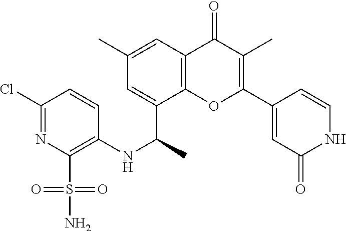 [SMILES: Cc1cc([C@@H](C)Nc2ccc(Cl)nc2S(N)(=O)=O)c2oc(-c3cc[nH]c(=O)c3)c(C)c(=O)c2c1] | 501 [M + H]⁺ |
| 238C | 6-Chloro-3-[[(1R)-1-(3- cyano-6-methyl-4-oxo-2- phenyl-chromen-8- yl)ethyl]amino]pyridine-2- sulfonamide | 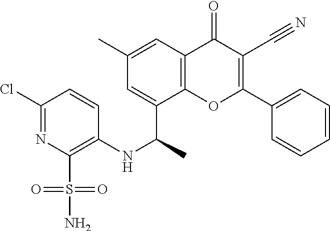 [SMILES: Cc1cc([C@@H](C)Nc2ccc(Cl)nc2S(N)(=O)=O)c2oc(-c3ccccc3)c(C#N)c(=O)c2c1] | 495 [M + H]⁺ |
| 239C | 6-Chloro-3-[[(1R)-1-[3- isoxazol-4-y1-6-methyl-2-(1- methylpyrazol-4-yl)-4-oxo- chromen-8- yl]ethyl]amino]pyridine-2- sulfonamide | 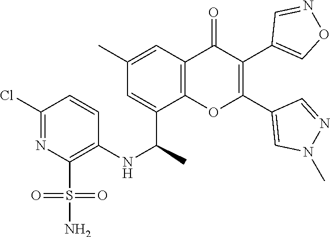 [SMILES: Cc1cc([C@@H](C)Nc2ccc(Cl)nc2S(N)(=O)=O)c2oc(-c3cnn(C)c3)c(-c3cnoc3)c(=O)c2c1] | 541 [M + H]⁺ |

### Example 146C: N-[[6-Chloro-3-[[(1R)-1-[3,6-dimethyl-2-(1-methylpyrazol-4-yl)-4-oxo-chromen-8-yl]ethyl]amino]-2-pyridyl]sulfonyl]acetamide

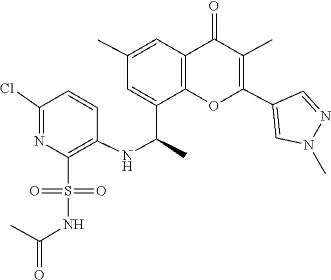 [SMILES: CC(=O)NS(=O)(=O)c1nc(Cl)ccc1N[C@H](C)c1cc(C)cc2c(=O)c(C)c(-c3cnn(C)c3)oc12]

A solution of 6-chloro-3-[[(1R)-1-[3,6-dimethyl-2-(1-methylpyrazol-4-yl)-4-oxo-chromen-8-yl]ethyl]amino]pyridine-2-sulfonamide trifluoroacetate (30 mg, 0.05 mmol) and DIPEA (23.8 mg, 0.18 mmol) in DCM (1 mL) was treated with acetic anhydride (9.4 mg, 0.09 mmol) at rt. The reaction was allowed to stir for 2 h. The reaction was diluted with water (20 mL) and extracted with DCM (3×60 mL). The combined organic layers were washed with brine (3×15 mL), dried over Na₂SO₄, filtered, and concentrated under reduced pressure. The residue was purified by reversed phase chromatography on C18 eluted with 50% to 60% ACN in water (with 0.1% formic acid) to give the title compound (10.6 mg, 32%) as a white solid. ES/MS m/z 530 (M+H).

The following compounds in Table 86 were made in a similar way as described for N-[[6-chloro-3-[[(1R)-1-[3,6-dimethyl-2-(1-methylpyrazol-4-yl)-4-oxo-chromen-8-yl]ethyl]amino]-2-pyridyl]sulfonyl]acetamide. Various methods were used to purify the compounds, which would be apparent to one skilled in the art.

TABLE 86

| Example | | | MS ES+ |
|---|---|---|---|
| # | Chemical Name | Structure | m/z |
| 147C | N-[[6-Chloro-3-[[(1R)- 1-[3,6-dimethyl-2-(2- methylindazol-5-yl)-4- oxo-chromen-8- yl]ethyl]amino]-2- pyridyl]sulfonyl] acetamide | 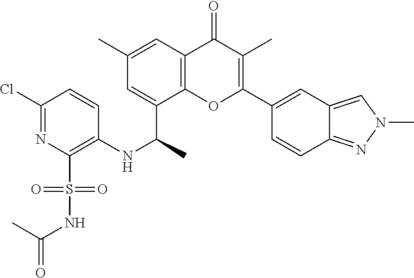 [SMILES: CC(=O)NS(=O)(=O)c1nc(Cl)ccc1N[C@H](C)c1cc(C)cc2c(=O)c(C)c(-c3ccc4nn(C)cc4c3)oc12] | 580 [M + H]⁺ |
| 148C | N-[6-Chloro-3-[[(1R)- 1-[3,6-dimethyl-4-oxo- 2-(2-pyridyl)chromen- 8-yl]ethyl]amino]-2- pyridyl]sulfonyl] acetamide | 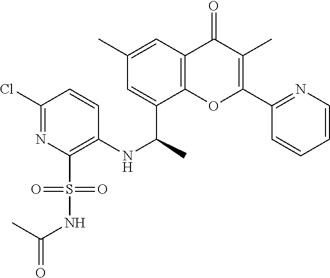 [SMILES: CC(=O)NS(=O)(=O)c1nc(Cl)ccc1N[C@H](C)c1cc(C)cc2c(=O)c(C)c(-c3ccccn3)oc12] | 527 [M + H]⁺ |
| 149C | N-[6-Chloro-3-[(1R)- 1-[3,6-dimethyl-4-oxo- 2-(3-pyridyl)chromen- 8-yl]ethyl]amino]-2- pyridyl]sulfonyl] acetamide | 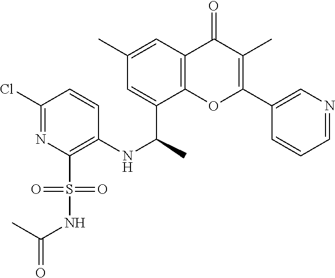 [SMILES: CC(=O)NS(=O)(=O)c1nc(Cl)ccc1N[C@H](C)c1cc(C)cc2c(=O)c(C)c(-c3cccnc3)oc12] | 527 [M + H]⁺ |
| 150C | N-[6-Chloro-3-[(1R)- 1-[3,6-dimethyl-2-(2- methylindazol-5-y1)-4- oxo-chromen-8- yl]ethyl]amino]-2- pyridyl]sulfonyl] acetamide | 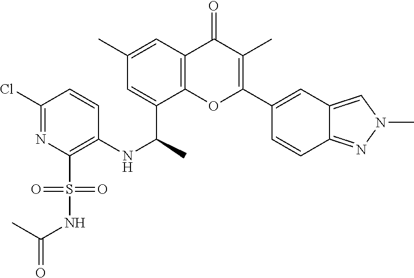 | 566 [M + H]⁺ |
| 151C | N-[6-Chloro-3-[(1R)- 1-(3-isoxazol-4-yl-6- methyl-4-oxo-2- phenyl-chromen-8- yl)ethyl]amino]-2- pyridyl]sulfonyl] acetamide | 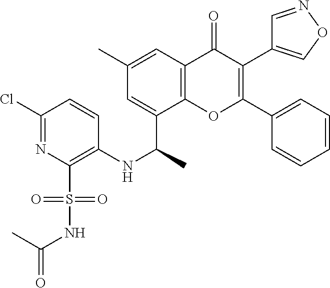 [SMILES: CC(=O)NS(=O)(=O)c1nc(Cl)ccc1N[C@H](C)c1cc(C)cc2c(=O)c(-c3cnoc3)c(-c3ccccc3)oc12] | 579 [M + H]⁺ |

### Example 152C: 3-[6-Chloro-3-[[(1R)-1-[3,6-dimethyl-2-[1-(1-methylsulfonylazetidin-3-yl)pyrazol-4-yl]-4-oxo-chromen-8-yl]ethyl]amino]-2-pyridyl]-4H-1,2,4-oxadiazol-5-one

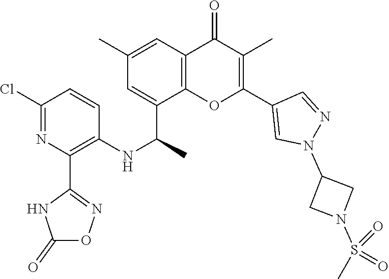 [SMILES: Cc1cc([C@@H](C)Nc2ccc(Cl)nc2-c2noc(=O)[nH]2)c2oc(-c3cnn(C4CN(S(C)(=O)=O)C4)c3)c(C)c(=O)c2c1]

A solution of 3-[3-[[(1R)-1-[2-[1-(539zetidine-3-yl)pyrazol-4-yl]-3,6-dimethyl-4-oxo-chromen-8-yl]ethyl]amino]-6-chloro-2-pyridyl]-4H-1,2,4-oxadiazol-5-one (30 mg, 0.06 mmol) in DCM (1 mL) was treated with triethylamine (11 mg, 0.11 mmol) and methanesulfonyl chloride (9.7 mg, 0.08 mmol). The reaction was stirred at rt for 1 h. The reaction was concentrated under reduced pressure and the residue purified by preparative TLC eluted with 4% MeOH in DCM to give the title compound (23.8 mg, 69%) as a pale tan foam. ES-MS m/z 612 (M+H).

The following compound in Table 87 was made in a similar way as described for 3-[6-chloro-3-[[(1R)-1-[3,6-dimethyl-2-[1-(1-methylsulfonylazetidin-3-yl)pyrazol-4-yl]-4-oxo-chromen-8-yl]ethyl]amino]-2-pyridyl]-4H-1,2,4-oxadiazol-5-one. Various methods were used to purify the compound, which would be apparent to one skilled in the art.

TABLE 87

| Example | | | MS ES+ |
|---|---|---|---|
| # | Chemical Name | Structure | m/z |
| 153C | 3-[6-Chloro-3-[(1R)- 1-[2-[1-[1-(2- methoxyacetyl)azetidin- 3-y1]pyrazol-4-y1]-3,6- dimethyl-4-oxo- chromen-8- ylethyl]amino]-2- pyridyl]-4H-1,2,4- oxadiazol-5-one [HYW-00002-282] | 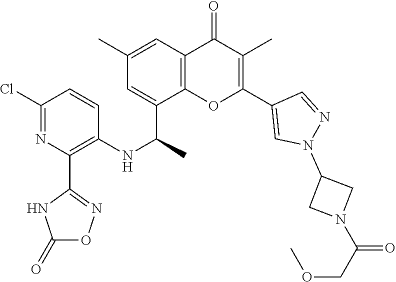 [SMILES: COCC(=O)N1CC(n2cc(-c3oc4c([C@@H](C)Nc5ccc(Cl)nc5-c5noc(=O)[nH]5)cc(C)cc4c(=O)c3C)cn2)C1] | 606 [M + H]⁺ |

### Example 261A: 8-[(1R)-1-(2-Dimethoxyphosphorylanilino)ethyl]-3,6-dimethyl-2-phenyl-chromen-4-one

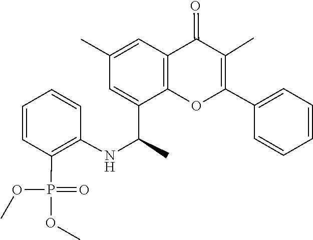 [SMILES: COP(=O)(OC)c1ccccc1N[C@H](C)c1cc(C)cc2c(=O)c(C)c(-c3ccccc3)oc12]

A mixture of 8-[(1R)-1-(2-dimethoxyphosphorylanilino)ethyl]-2-ethylsulfanyl-3,6-dimethyl-chromen-4-one (26 mg, 0.06 mmol), phenylboronic acid (21 mg, 0.17 mmol), copper(I) thiophene-2-carboxylate (16 mg, 0.09 mmol), and tetrakis(triphenylphosphine)palladium(0) (6.5 mg, 0.06 mmol) were combined in ethanol (1 mL). The reaction was degassed for 5 min using argon gas and then stirred at 65° C. for almost 4 h. The reaction was adsorbed onto celite and purified by reversed phase chromatography eluted with 10% to 65% ACN in 10 mM NH₄HCO₃ (with 5% MeOH) to give the title compound (4.3 mg, 16%. MS ES+ m/z 478 [M+H]⁺.

### Example 262A: 6-Chloro-3-[[(1R)-1-(3-cyano-6-methyl-4-oxo-2-phenyl-chromen-8-yl)ethyl]amino]-N-methylsulfonyl-pyridine-2-carboxamide

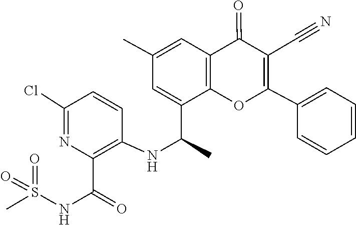 [SMILES: Cc1cc([C@@H](C)Nc2ccc(Cl)nc2C(=O)NS(C)(=O)=O)c2oc(-c3ccccc3)c(C#N)c(=O)c2c1]

Combined 6-chloro-3-[[(1R)-1-(3-iodo-6-methyl-4-oxo-2-phenyl-chromen-8-yl)ethyl]amino]-N-methylsulfonyl-pyridine-2-carboxamide (20 mg, 0.03 mmol) and copper(I) cyanide (5.6 mg, 0.06 mmol) in 1 mL of DMF and stirred at 100° C. overnight. The reaction was allowed to cool to rt and diluted with 10 mL of DCM and 10 mL of saturated aqueous NaHCO₃. After removal of the organic layer, the remaining aqueous layer was extracted with DCM (3×5 mL). The organics were passed through a phase separator and concentrated. The residue was purified by reversed phase chromatography eluted with 10% to 100% ACN (with 0.1% formic acid) in water (with 0.1% formic acid) to give the title compound (2.9 mg, 17%). MS ES+ m/z 537 [M+H]⁺.

The following compounds in Table 88 were made in a similar way as described for 6-chloro-3-[[(1R)-1-(3-cyano-6-methyl-4-oxo-2-phenyl-chromen-8-yl)ethyl]amino]-N-methylsulfonyl-pyridine-2-carboxamide. Various methods were used to purify the compounds, which would be apparent to one skilled in the art.

TABLE 88

| Example | | | MS ES+ |
|---|---|---|---|
| # | Chemical Name | Structure | m/z |
| 154C | 8-[(1R)-1-[[6-Chloro- 2-(5-oxo-4H-1,2,4- oxadiazol-3-y1)-3- pyridyl]amino]ethyl]-6- methyl-4-oxo-2-(2- pyridyl)chromene-3- carbonitrile | 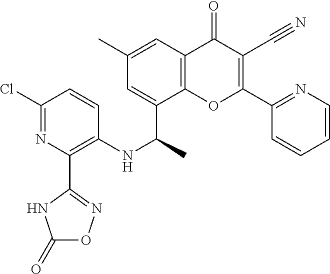 [SMILES: Cc1cc([C@@H](C)Nc2ccc(Cl)nc2-c2noc(=O)[nH]2)c2oc(-c3ccccn3)c(C#N)c(=O)c2c1] | 501 [M + H]⁺ |
| 155C | 8-[(1R)-1-[(6-Chloro- 2-methyl-3- pyridyl)amino]ethyl]-6- methyl-4-oxo-2-(3- pyridyl)chromene-3- carbonitrile | 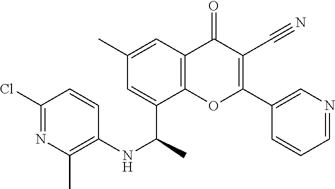 [SMILES: Cc1cc([C@@H](C)Nc2ccc(Cl)nc2C)c2oc(-c3cccnc3)c(C#N)c(=O)c2c1] | 431 [M + H]⁺ |
| 156C | 8-[(1R)-1-[[2-(2- Fluorophenyl)-3- pyridyl]amino]ethyl]-6- methyl-4-oxo-2-(3- pyridyl)chromene-3- carbonitrile | 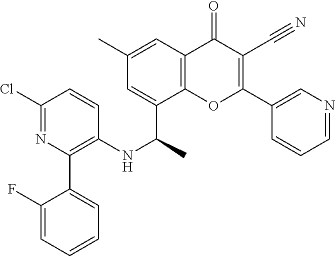 | 477 [M + H]⁺ |

### Example 263A: 6-Chloro-3-[[(1R)-1-(6-methyl-3-oxazol-4-yl-4-oxo-2-phenyl-chromen-8-yl)ethyl]amino]-N-methylsulfonyl-pyridine-2-carboxamide

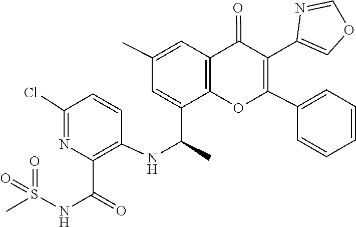 [SMILES: Cc1cc([C@@H](C)Nc2ccc(Cl)nc2C(=O)NS(C)(=O)=O)c2oc(-c3ccccc3)c(-c3cocn3)c(=O)c2c1]

Combined 6-chloro-3-[[(1R)-1-(3-iodo-6-methyl-4-oxo-2-phenyl-chromen-8-yl)ethyl]amino]-N-methylsulfonyl-pyridine-2-carboxamide (50 mg, 0.08 mmol), [1,1′-bis(diphenylphosphino)ferrocene]dichloropalladium(II) (17 mg, 0.02 mmol), and 4-(4,4,5,5-tetramethyl-1,3,2-dioxaborolan-2-yl)oxazole (46 mg, 0.02 mmol) in 1,4-dioxane (1.5 mL) and 2 M aqueous Na₂CO₃. The suspension was degassed with argon for 5 min and then allowed to stir at 100° C. for 1 h. The reaction was allowed to cool and diluted with DCM (10 mL) and 5 mL of saturated aqueous NH₄Cl. Extracted with DCM (3×10 mL). The organic layers were combined, passed through a phase separator frit and concentrated. The residue was purified by reversed phase chromatography eluted with 10% to 100% ACN (with 0.1% formic acid) in water (with 0.1% formic acid) to give the title compound (3.9 mg, 9%). MS ES+ m/z 579 [M+H]⁺.

The following compound in Table 89 was made in a similar way as described for 6-chloro-3-[[(1R)-1-(6-methyl-3-oxazol-4-yl-4-oxo-2-phenyl-chromen-8-yl)ethyl]amino]-N-methylsulfonyl-pyridine-2-carboxamide. Various methods were used to purify the compound, which would be apparent to one skilled in the art.

TABLE 89

| Example | | | MS ES+ |
|---|---|---|---|
| # | Chemical Name | Structure | m/z |
| 157C | 8-[(1R)-1-[[2-(2- Fluorophenyl)-3- pyridyl]amino]ethyl]-3- isoxazol-4-y1-6-methyl- 2-(3-pyridyl)chromen- 4-one | 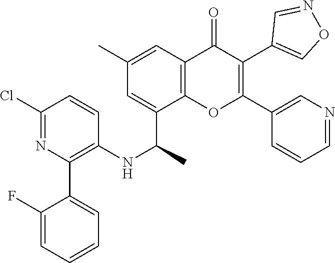 | 519 [M + H]⁺ |

### Example 158C: 8-[(1R)-1-[[2-(2-Fluorophenyl)-3-pyridyl]amino]ethyl]-6-methyl-3-oxazol-4-yl-2-(3-pyridyl)chromen-4-one

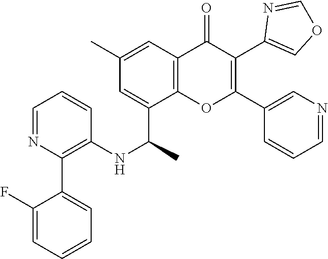 [SMILES: Cc1cc([C@@H](C)Nc2cccnc2-c2ccccc2F)c2oc(-c3cccnc3)c(-c3cocn3)c(=O)c2c1]

Combined 8-[(1R)-1-[[2-(2-fluorophenyl)-3-pyridyl]amino]ethyl]-3-iodo-6-methyl-2-(3-pyridyl)chromen-4-one (200 mg, 0.35 mmol), 4-(4,4,5,5-tetramethyl-1,3,2-dioxaborolan-2-yl)oxazole (135 mg, 0.69 mmol), cesium carbonate (339 mg, 1.04 mmol), and 1,1′-bis(di-tert-butylphosphino)ferrocene palladium dichloride (56.4 mg, 0.09 mmol) in 10% aqueous 1,4-dioxane. The mixture was sparged for 5 min with argon and then stirred at 65° C. for 3 h. After cooling, the reaction was filtered through diatomaceous earth and the solids washed with DCM. The filtrate was concentrated under reduced pressure and the residue purified by silica gel chromatography eluted with 0% to 10% MeOH in DCM to give the title compound (89.7 mg, 47% as a tan foam. ES/MS m/z 519 (M+H).

The following compound in Table 90 was made in a similar way as described for 8-[(1R)-1-[[2-(2-fluorophenyl)-3-pyridyl]amino]ethyl]-6-methyl-3-oxazol-4-yl-2-(3-pyridyl)chromen-4-one. Various methods were used to purify the compound, which would be apparent to one skilled in the art.

TABLE 90

| Example | | | MS ES+ |
|---|---|---|---|
| # | Chemical Name | Structure | m/z |
| 159C | 8-[(1R)-1-[[2-(2- Fluorophenyl)-3- pyridyl]amino]ethyl]-6- methyl-3-(3- methylisoxazol-4-yl)-2- (3-pyridyl)chromen-4- one | 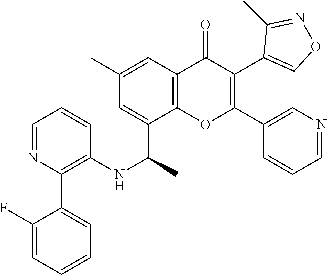 [SMILES: Cc1cc([C@@H](C)Nc2cccnc2-c2ccccc2F)c2oc(-c3cccnc3)c(-c3conc3C)c(=O)c2c1] | 533 [M + H]⁺ |

### Example 264A: 8-[[(1R)-1-[3,6-Dimethyl-4-oxo-2-(3-pyridyl)chromen-8-yl]ethyl]amino]-3,4-dihydro-2H-isoquinolin-1-one

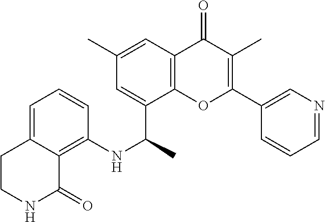 [SMILES: Cc1cc([C@@H](C)Nc2cccc3c2C(=O)NCC3)c2oc(-c3cccnc3)c(C)c(=O)c2c1]

A 20 mL vial was charged with 8-[[(1R)-1-[3,6-dimethyl-4-oxo-2-(3-pyridyl)chromen-8-yl]ethyl]amino]-2-[(4-methoxyphenyl)methyl]-3,4-dihydroisoquinolin-1-one (145 mg, 0.26 mmol) and TFA (5 mL, 60 mmol) and stirred at 80° C. for 4 days. The reaction was allowed to cool and concentrated. The residue was taken up in EtOAc three times and concentrated. The residue was dissolved in EtOAc, washed with water (3×), and saturated aqueous sodium chloride. The organic layer was concentrated and the residue purified by reversed phase chromatography eluted with 10% to 100% MeOH in 10 mM aqueous NH₄HCO₃ to give the title compound (47.3 mg, 42%) as a white solid. MS ES+ m/z 440 [M+H]⁺.

### Example 265A: S-(2-Pyridylmethyl) 2-[[(1R)-1-(3,6-dimethyl-4-oxo-2-phenyl-chromen-8-yl)ethyl]amino]benzenecarbothioate

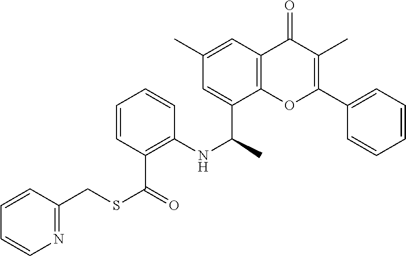 [SMILES: Cc1cc([C@@H](C)Nc2ccccc2C(=O)SCc2ccccn2)c2oc(-c3ccccc3)c(C)c(=O)c2c1]

A solution of 2-[[(1R)-1-(3,6-dimethyl-4-oxo-2-phenyl-chromen-8-yl)ethyl]amino]benzoic acid (204 mg, 0.49 mmol) in DCM (4.9 mL) was flushed with nitrogen gas and cooled in a dry ice/acetone bath for 5 min. Treated the reaction with isobutyl chloroformate (67.4 mg, 0.49 mmol) and then triethylamine (150 mg, 1.48 mmol). The reaction was removed from the bath, placed in an ice bath, and treated with pyridine-2-ylmethanethiol (61.8 mg, 0.49 mmol). The reaction was removed from the cooling bath and allowed to warm to rt. After 40 min, the reaction was placed into a −20° C. freezer overnight. The reaction was allowed to warm to rt over 30 min. The reaction was diluted with 50% aqueous NaHCO₃ and the layers separated. The organic layer was concentrated and purified by silica gel chromatography eluted with 0% to 100% EtOAc in heptane to give the title compound (177.5 mg, 69%). MS ES+ m/z 521 [M+H]⁺.

### Example 160C: 8-[(1R)-1-[[6-Chloro-2-(methylamino)-3-pyridyl]amino]ethyl]-3,6-dimethyl-2-(3-pyridyl)chromen-4-one

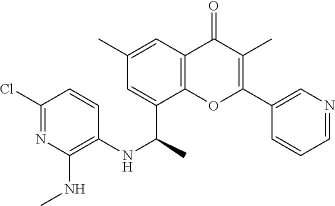 [SMILES: CNc1nc(Cl)ccc1N[C@H](C)c1cc(C)cc2c(=O)c(C)c(-c3cccnc3)oc12]

A vial containing 8-[(1R)-1-[(6-chloro-2-fluoro-3-pyridyl)amino]ethyl]-3,6-dimethyl-2-(3-pyridyl)chromen-4-one (50 mg, 0.12 mmol) and DMF (2 mL) was treated with methylamine 2.0 M in THF, 0.14 mL, 0.28 mmol) and cesium carbonate (0.12 g, 0.35 mmol). The vial was sealed and the reaction stirred at 100° C. for 15 h. After cooling, the reaction was filtered and the filtrate purified by reversed phase chromatography on a CSH column eluted with 25% to 57% ACN in water (with 0.1% formic acid) to give the title compound (4.3 mg, 8%) as a white solid. ES/MS m/z 435 (M+H).

The following compounds in Table 91 were made in a similar way as described for 8-[(1R)-1-[[6-chloro-2-(methylamino)-3-pyridyl]amino]ethyl]-3,6-dimethyl-2-(3-pyridyl)chromen-4-one. Various methods were used to purify the compounds, which would be apparent to one skilled in the art.

TABLE 91

| Example | | | MS ES+ |
|---|---|---|---|
| # | Chemical Name | Structure | m/z |
| 161C | 8-[(1R)-1-[[6-Chloro- 2-(4-methylpiperazin- 1-y1)-3- pyridyl]amino]ethyl]- 3,6-dimethyl-2-(3- pyridyl)chromen-4-one | 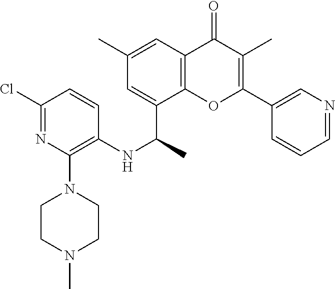 [SMILES: Cc1cc([C@@H](C)Nc2ccc(Cl)nc2N2CCN(C)CC2)c2oc(-c3cccnc3)c(C)c(=O)c2c1] | 504 [M + H]⁺ |
| 162C | 8-[(1R)-1-[[6-Chloro- 2-(4,4-difluoro-1- piperidyl)-3- pyridyl]amino]ethyl]- 3,6-dimethyl-2-(3- pyridyl)chromen-4-one | 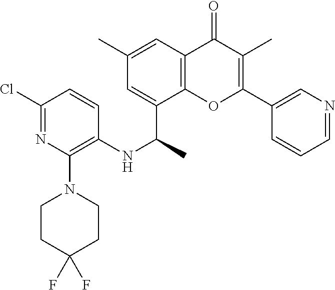 [SMILES: Cc1cc([C@@H](C)Nc2ccc(Cl)nc2N2CCC(F)(F)CC2)c2oc(-c3cccnc3)c(C)c(=O)c2c1] | 525 [M + H]⁺ |
| 163C | 8-[(1R)-1-[[6-Chloro- 2-(1-piperidyl)-3- pyridyl]amino]ethyl]- 3,6-dimethyl-2-(3- pyridyl)chromen-4-one | 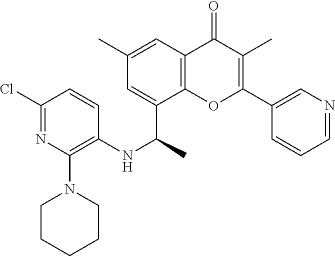 [SMILES: Cc1cc([C@@H](C)Nc2ccc(Cl)nc2N2CCCCC2)c2oc(-c3cccnc3)c(C)c(=O)c2c1] | 489 [M + H]⁺ |
| 164C | 8-[(1R)-1-[[6-Chloro- 2-(dimethylamino)-3- pyridyl]amino]ethyl]- 3,6-dimethyl-2-(3- pyridyl)chromen-4-one | 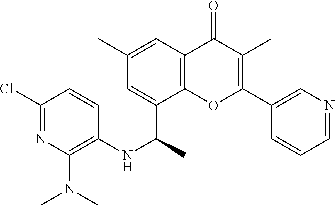 [SMILES: Cc1cc([C@@H](C)Nc2ccc(Cl)nc2N(C)C)c2oc(-c3cccnc3)c(C)c(=O)c2c1] | 449 [M + H]⁺ |
| 165C | 8-[(1R)-1-[(6-Chloro- 2-morpholino-3- pyridyl)amino]ethyl]- 3,6-dimethyl-2-(3- pyridyl)chromen-4-one | 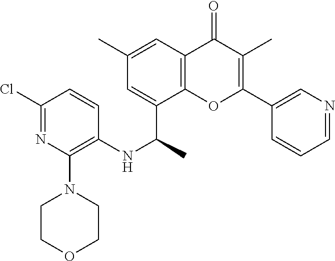 [SMILES: Cc1cc([C@@H](C)Nc2ccc(Cl)nc2N2CCOCC2)c2oc(-c3cccnc3)c(C)c(=O)c2c1] | 491 [M + H]⁺ |

### Example 1B: 8-[(1R)-1-[(5-Chloro-3-pyridyl)amino]ethyl]-3,6-dimethyl-2-phenyl-chromen-4-one

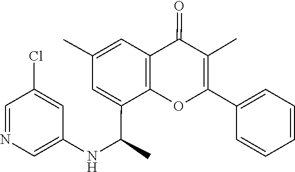 [SMILES: Cc1cc([C@@H](C)Nc2cncc(Cl)c2)c2oc(-c3ccccc3)c(C)c(=O)c2c1]

A mixture of tris(dibenzylideneacetone)dipalladium(0) (0.047 g, 0.051 mmol) and 4,5-bis(diphenylphosphino)-9,9-dimethylxanthene (0.089 g, 0.15 mmol) in toluene (18 mL) was stirred at rt for 10 min. 8-[(1R)-1-aminoethyl]-3,6-dimethyl-2-phenyl-chromen-4-one (0.15 g, 0.51 mmol), 3-bromo-5-chloropyridine (0.098 g, 0.51 mmol), and Cs₂CO₃ (0.33 g, 1.0 mmol) were then added to the mixture, which was flushed with nitrogen. The reaction was sealed and heated to 115° C. overnight. The mixture was filtered over a pad of diatomaceous earth and the solids were rinsed with DCM. The filtrate was concentrated and the residue was purified by reverse phase chromatography eluted with a gradient of 20 to 100% ACN in aqueous NH₄HCO₃ (10 mM, plus 5% MeOH) to give the title compound (0.062 g, 30%). MS ES+ m/z 405 [M+H]⁺.

The following compounds in Table 92 were made in a similar way as described for 8-[(1R)-1-[(5-chloro-3-pyridyl)amino]ethyl]-3,6-dimethyl-2-phenyl-chromen-4-one. Various methods were used to purify the compounds, which would be apparent to one skilled in the art.

TABLE 92

| Example | | | MS ES+ |
|---|---|---|---|
| # | Chemical Name | Structure | m/z |
| 2B | 8-[(1R)-1-[(4-Chloro- 3-pyridyl)amino]ethyl]- 3,6-dimethyl-2-phenyl- chromen-4-one | 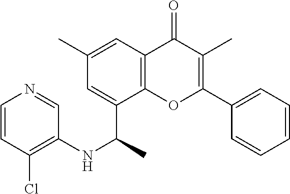 [SMILES: Cc1cc([C@@H](C)Nc2cnccc2Cl)c2oc(-c3ccccc3)c(C)c(=O)c2c1] | 405 [M + H]⁺ |
| 3B | 8-[(1R)-1-[(6- Cyclopropyl-3- pyridyl)amino]ethyl]- 3,6-dimethyl-2-phenyl- chromen-4-one | 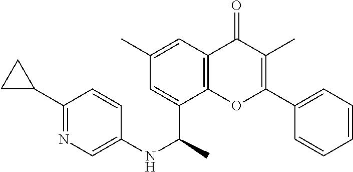 [SMILES: Cc1cc([C@@H](C)Nc2ccc(C3CC3)nc2)c2oc(-c3ccccc3)c(C)c(=O)c2c1] | 411 [M + H]⁺ |
| 4B | 8-[(1R)-1-[(2-Fluoro-3- pyridyl)amino]ethyl]- 3,6-dimethyl-2-phenyl- chromen-4-one 2,2,2- trifluoroacetic acid | 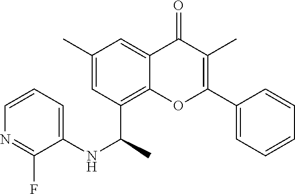 [SMILES: Cc1cc([C@@H](C)Nc2cccnc2F)c2oc(-c3ccccc3)c(C)c(=O)c2c1] | 389 [M + H]⁺ |
| | | 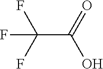 | |
| 5B | 8-[(1R)-1-[(2-Chloro- 3-pyridyl)amino]ethyl]- 3,6-dimethyl-2-phenyl- chromen-4-one | 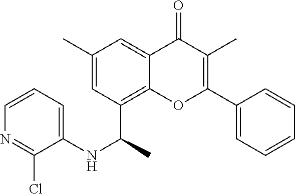 [SMILES: Cc1cc([C@@H](C)Nc2cccnc2Cl)c2oc(-c3ccccc3)c(C)c(=O)c2c1] | 405 [M + H]⁺ |
| 6B | 3,6-Dimethyl-8-[(1R)- 1-[(2-methyl-3- pyridyl)amino]ethyl]-2- phenyl-chromen-4-one | 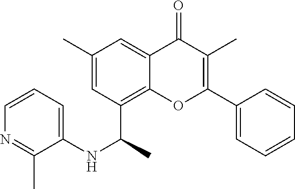 [SMILES: Cc1cc([C@@H](C)Nc2cccnc2C)c2oc(-c3ccccc3)c(C)c(=O)c2c1] | 385 [M + H]⁺ |
| 7B | 8-[(1R)-1-[(2- Cyclopropyl-3- pyridyl)amino]ethyl]- 3,6-dimethyl-2-phenyl- chromen-4-one 2,2,2- trifluoroacetic acid | 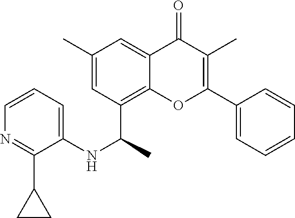 [SMILES: Cc1cc([C@@H](C)Nc2cccnc2C2CC2)c2oc(-c3ccccc3)c(C)c(=O)c2c1] | 411 [M + H]⁺ |
| | | 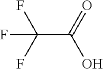 | |
| 8B | 8-[(1R)-1-[(6-Chloro- 3-pyridyl)amino]ethyl]- 2-[6-(difluoromethyl)- 2-pyridy1]-3,6- dimethyl-chromen-4- one | 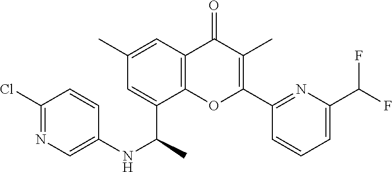 [SMILES: Cc1cc([C@@H](C)Nc2ccc(Cl)nc2)c2oc(-c3cccc(C(F)F)n3)c(C)c(=O)c2c1] | 456 [M + H]⁺ |
| 9B | 3,6-Dimethyl-8-[(1R)- 1-[(2-methyl-3- pyridyl)amino]ethyl]-2- (3-pyridyl)chromen-4- one | 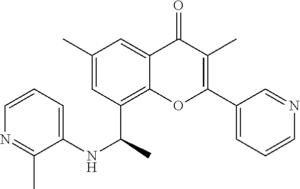 [SMILES: Cc1cc([C@@H](C)Nc2cccnc2C)c2oc(-c3cccnc3)c(C)c(=O)c2c1] | 386 [M + H]⁺ |
| 10B | 8-[(1R)-1-[(6-Chloro- 2-methyl-3- pyridyl)amino]ethyl]- 3,6-dimethyl-2-(3- pyridyl)chromen-4-one | 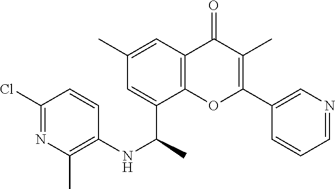 [SMILES: Cc1cc([C@@H](C)Nc2ccc(Cl)nc2C)c2oc(-c3cccnc3)c(C)c(=O)c2c1] | 420 [M + H]⁺ |
| 30B | 8-[(1R)-1-[(6-Chloro- 2-methyl-3- pyridyl)amino]ethyl]- 3,6-dimethyl-2-(2- methylindazol-5- yl)chromen-4-one | 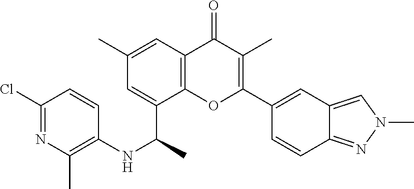 [SMILES: Cc1cc([C@@H](C)Nc2ccc(Cl)nc2C)c2oc(-c3ccc4nn(C)cc4c3)c(C)c(=O)c2c1] | 473 [M + H]⁺ |
| 31B | 8-[(1R)-1-[(6-Fluoro-2- methyl-3- pyridyl)amino]ethyl]- 3,6-dimethyl-2-(3- pyridyl)chromen-4-one | 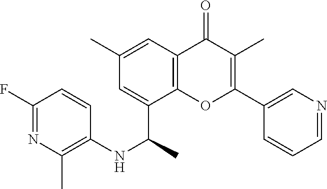 [SMILES: Cc1cc([C@@H](C)Nc2ccc(F)nc2C)c2oc(-c3cccnc3)c(C)c(=O)c2c1] | 404 [M + H]⁺ |
| 32B¹ | 8-[(1R)-1-[(6-Chloro- 2-methyl-3- pyridyl)amino]ethyl]-2- (5-fluoro-3-pyridyl)- 3,6-dimethyl-chromen- 4-one | 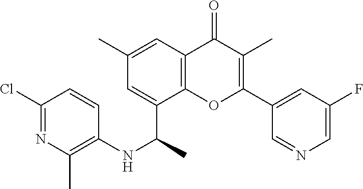 [SMILES: Cc1cc([C@@H](C)Nc2ccc(Cl)nc2C)c2oc(-c3cncc(F)c3)c(C)c(=O)c2c1] | 438 [M + H]⁺ |
| 33B | 8-[(1R)-1-[(2,6- Dichloro-3- pyridyl)amino]ethyl]- 3,6-dimethyl-2-(3- pyridyl)chromen-4-one | 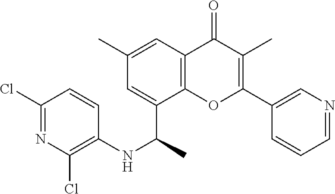 [SMILES: Cc1cc([C@@H](C)Nc2ccc(Cl)nc2Cl)c2oc(-c3cccnc3)c(C)c(=O)c2c1] | 440 [M + H]⁺ |
| 34B¹ | 8-[(1R)-1-[(6-Chloro- 2-methyl-3- pyridyl)amino]ethyl]-2- (3-fluorophenyl)-3,6- dimethyl-chromen-4- one | 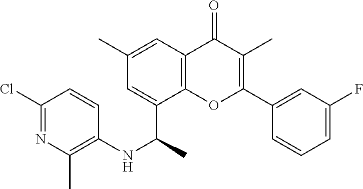 [SMILES: Cc1cc([C@@H](C)Nc2ccc(Cl)nc2C)c2oc(-c3cccc(F)c3)c(C)c(=O)c2c1] | 437 [M + H]⁺ |
| 35B | 8-[(1R)-1-(4-Chloro-2- methyl-anilino)ethyl]- 3,6-dimethyl-2-(3- pyridyl)chromen-4-one | 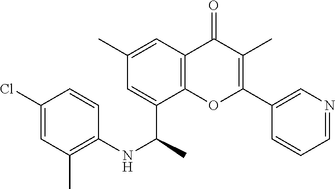 [SMILES: Cc1cc([C@@H](C)Nc2ccc(Cl)cc2C)c2oc(-c3cccnc3)c(C)c(=O)c2c1] | 419 [M + H]⁺ |
| 36B | 8-[(1R)-1-[(2-Chloro- 6-methyl-3- pyridyl)amino]ethyl]- 3,6-dimethyl-2-(3- pyridyl)chromen-4-one | 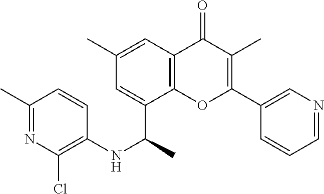 [SMILES: Cc1cc([C@@H](C)Nc2ccc(C)nc2Cl)c2oc(-c3cccnc3)c(C)c(=O)c2c1] | 420 [M + H]⁺ |
| 37B² | 3,6-Dimethyl-8-[(1R)- 1-[2-(1- methylcyclopropyl)anil ino]ethyl]-2-(3- pyridyl)chromen-4-one | 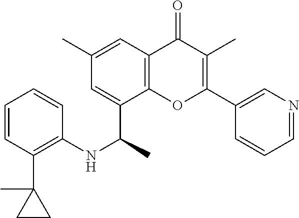 [SMILES: Cc1cc([C@@H](C)Nc2ccccc2C2(C)CC2)c2oc(-c3cccnc3)c(C)c(=O)c2c1] | 425 [M + H]⁺ |
| 38B¹ | 8-[(1R)-1-[(6-Chloro- 2-methyl-3- pyridyl)amino]ethyl]-2- (2,4-difluorophenyl)- 3,6-dimethyl-chromen- 4-one | 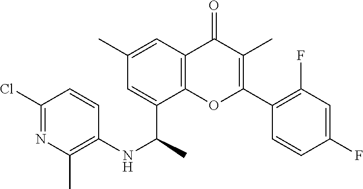 [SMILES: Cc1cc([C@@H](C)Nc2ccc(Cl)nc2C)c2oc(-c3ccc(F)cc3F)c(C)c(=O)c2c1] | 455 [M + H]⁺ |
| 39B³ | 8-[(1R)-1-(2-Chloro-4- methyl-anilino)ethyl]- 3,6-dimethyl-2-(3- pyridyl)chromen-4-one | 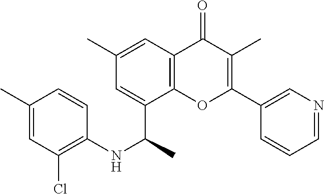 [SMILES: Cc1ccc(N[C@H](C)c2cc(C)cc3c(=O)c(C)c(-c4cccnc4)oc23)c(Cl)c1] | 419 [M + H]⁺ |
| 40B | 3,6-Dimethyl-2-phenyl- 8-[(1R)-1-[[6- (trifluoromethyl)-3- pyridyl]amino]ethyl] chromen-4-one | 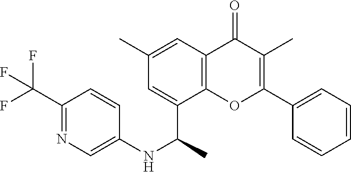 [SMILES: Cc1cc([C@@H](C)Nc2ccc(C(F)(F)F)nc2)c2oc(-c3ccccc3)c(C)c(=O)c2c1] | 439 [M + H]⁺ |
| 41B | 5-[[(1R)-1-(3,6- Dimethyl-4-oxo-2- phenyl-chromen-8- yl)ethyl]amino] pyridine-2-carbonitrile | 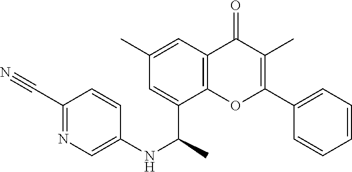 [SMILES: Cc1cc([C@@H](C)Nc2ccc(C#N)nc2)c2oc(-c3ccccc3)c(C)c(=O)c2c1] | 396 [M + H]⁺ |
| 42B¹ | 5-[8-[(1R)-1-[(6- Chloro-2-methyl-3- pyridyl)amino]ethyl]- 3,6-dimethyl-4-oxo- chromen-2-y1]-1- methyl-pyridin-2-one | 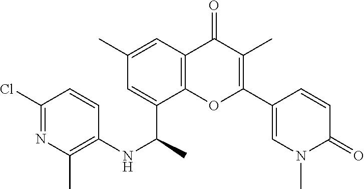 [SMILES: Cc1cc([C@@H](C)Nc2ccc(Cl)nc2C)c2oc(-c3ccc(=O)n(C)c3)c(C)c(=O)c2c1] | 450 [M + H]⁺ |
| 43B¹ | 4-[8-[(1R)-1-[(6- Chloro-2-methyl-3- pyridyl)amino]ethyl]- 3,6-dimethyl-4-oxo- chromen-2-y1]-1- methyl-pyridin-2-one | 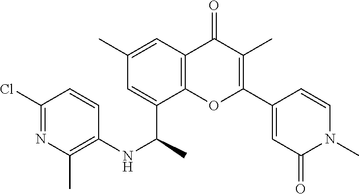 [SMILES: Cc1cc([C@@H](C)Nc2ccc(Cl)nc2C)c2oc(-c3ccn(C)c(=O)c3)c(C)c(=O)c2c1] | 450 [M + H]⁺ |
| 44B | 8-[(1R)-1-[(6-Chloro- 2-methyl-3- pyridyl)amino]ethyl]-3- methyl-2-(1- methylpyrazol-4-yl)-6- (trifluoromethyl) chromen-4-one | 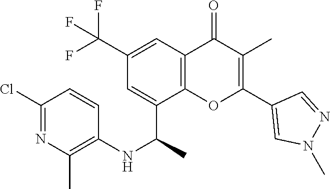 [SMILES: Cc1nc(Cl)ccc1N[C@H](C)c1cc(C(F)(F)F)cc2c(=O)c(C)c(-c3cnn(C)c3)oc12] | 477 [M + H]⁺ |
| 45B¹ | 8-[(1R)-1-[(6-Chloro- 2-methyl-3- pyridyl)amino]ethyl]- 3,6-dimethyl-2-(2- pyridyl)chromen-4-one | 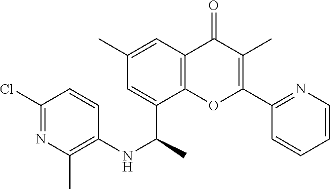 [SMILES: Cc1cc([C@@H](C)Nc2ccc(Cl)nc2C)c2oc(-c3ccccn3)c(C)c(=O)c2c1] | 420 [M + H]⁺ |
| 166C | 8-[(1R)-1-[[6-Chloro- 2-(2-oxa-6- azaspiro[3.3]heptan-6- y1)-3- pyridyl]amino]ethyl]- 3,6-dimethyl-2-(3- pyridyl)chromen-4-one | 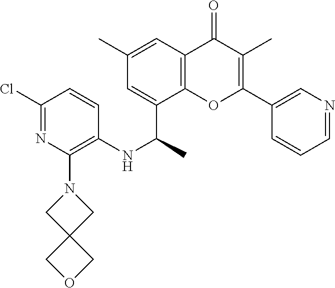 [SMILES: Cc1cc([C@@H](C)Nc2ccc(Cl)nc2N2CC3(COC3)C2)c2oc(-c3cccnc3)c(C)c(=O)c2c1] | 503 [M + H]⁺ |
| 167C | 8-[(1R)-1-[(5- Chloroimidazo[1,2- alpyridin-8- yl)amino]ethyl]-3,6- dimethyl-2-(3- pyridyl)chromen-4-one | 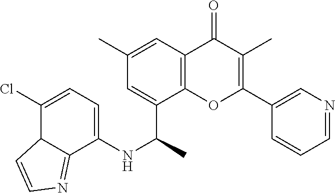 [SMILES: Cc1cc([C@@H](C)NC2=CC=C(Cl)C3C=CN=C23)c2oc(-c3cccnc3)c(C)c(=O)c2c1] | 445 [M + H]⁺ |
| 168C⁴ | 8-[(1R)-1-[[2- (Dimethylamino)-3- pyridyl]amino]ethyl]- 3,6-dimethyl-2-(3- pyridyl)chromen-4-one | 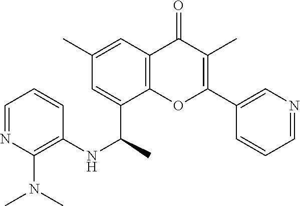 [SMILES: Cc1cc([C@@H](C)Nc2cccnc2N(C)C)c2oc(-c3cccnc3)c(C)c(=O)c2c1] | 415 [M + H]⁺ |
| 169C | 8-[(1R)-1-[(2,6- Dimethyl-3- pyridyl)amino]ethyl]- 3,6-dimethyl-2-(3- pyridyl)chromen-4-one | 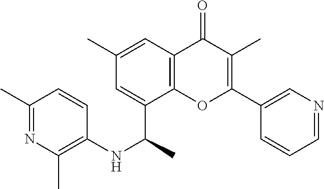 [SMILES: Cc1cc([C@@H](C)Nc2ccc(C)nc2C)c2oc(-c3cccnc3)c(C)c(=O)c2c1] | 400 [M + H]⁺ |
| 170C | 8-[(1R)-1-[(6-Chloro- 2-morpholino-3- pyridyl)amino]ethyl]- 3,6-dimethyl-2-(1- methylpyrazol-4- yl)chromen-4-one | 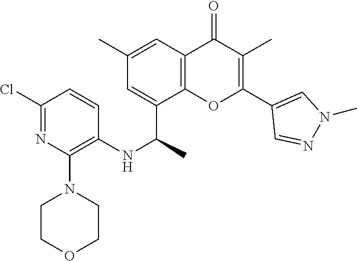 [SMILES: Cc1cc([C@@H](C)Nc2ccc(Cl)nc2N2CCOCC2)c2oc(-c3cnn(C)c3)c(C)c(=O)c2c1] | 494 [M + H]⁺ |
| 171C | 8-[(1R)-1-[[2-(4- Acetylpiperazin-1-yl)- 6-chloro-3- pyridyl]amino]ethyl]- 3,6-dimethyl-2-(3- pyridyl)chromen-4-one | 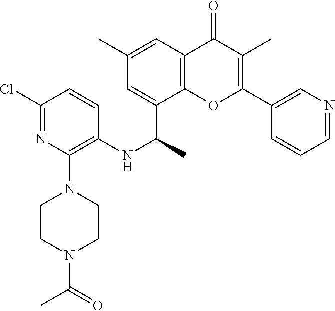 [SMILES: CC(=O)N1CCN(c2nc(Cl)ccc2N[C@H](C)c2cc(C)cc3c(=O)c(C)c(-c4cccnc4)oc23)CC1] | 532 [M + H]⁺ |
| 172C | 8-[(1R)-1-[[2- (Azetidin-1-yl)-6- chloro-3- pyridyl]amino]ethyl]- 3,6-dimethyl-2-(3- pyridyl)chromen-4-one | 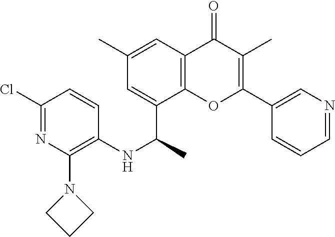 [SMILES: Cc1cc([C@@H](C)Nc2ccc(Cl)nc2N2CCC2)c2oc(-c3cccnc3)c(C)c(=O)c2c1] | 461 [M + H]⁺ |
| 173C | 8-[(1R)-1-[[6-Chloro- 2-(1-oxa-6- azaspiro[3.3]heptan-6- y1)-3- pyridyl]amino]ethyl]- 3,6-dimethyl-2-(3- pyridyl)chromen-4-one | 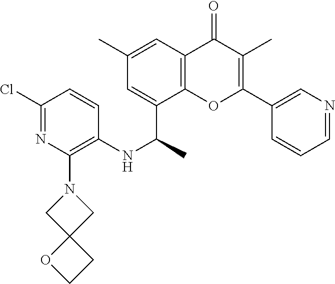 [SMILES: Cc1cc([C@@H](C)Nc2ccc(Cl)nc2N2CC3(CCO3)C2)c2oc(-c3cccnc3)c(C)c(=O)c2c1] | 503 [M + H]⁺ |
| 174C | 8-[(1R)-1-[[6-Chloro- 2-(3- morpholinoazetidin-1- y1)-3- pyridyl]amino]ethyl]- 3,6-dimethyl-2-(3- pyridyl)chromen-4-one | 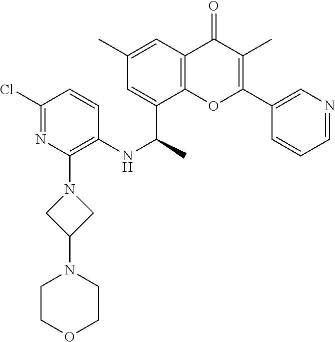 [SMILES: Cc1cc([C@@H](C)Nc2ccc(Cl)nc2N2CC(N3CCOCC3)C2)c2oc(-c3cccnc3)c(C)c(=O)c2c1] | 546 [M + H]⁺ |
| 175C | 8-[(1R)-1-[[6-Chloro- 2-(1, 1-dioxo-1,4- thiazinan-4-y1)-3- pyridyl]amino]ethyl]- 3,6-dimethyl-2-(3- pyridyl)chromen-4-one | 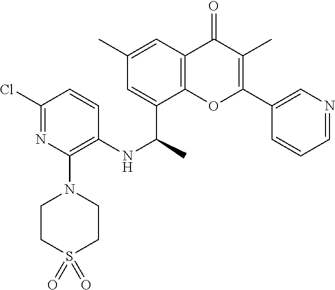 [SMILES: Cc1cc([C@@H](C)Nc2ccc(Cl)nc2N2CCS(=O)(=O)CC2)c2oc(-c3cccnc3)c(C)c(=O)c2c1] | 539 [M + H]⁺ |
| 176C | 8-[(1R)-1-[[6-Chloro- 2-(3,3-difluoroazetidin- 1-y1)-3- pyridyl]amino]ethyl]- 3,6-dimethyl-2-(3- pyridyl)chromen-4-one | 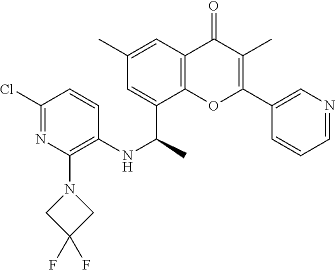 [SMILES: Cc1cc([C@@H](C)Nc2ccc(Cl)nc2N2CC(F)(F)C2)c2oc(-c3cccnc3)c(C)c(=O)c2c1] | 497 [M + H]⁺ |
| 177C | 8-[(1R)-1-[2-(3- Fluorophenyl)-3- pyridyl]amino]ethyl]- 3,6-dimethyl-2-phenyl- chromen-4-one | 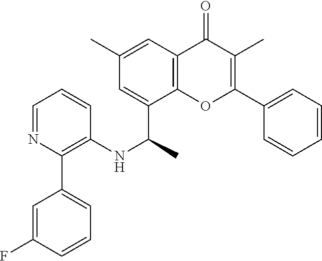 [SMILES: Cc1cc([C@@H](C)Nc2cccnc2-c2cccc(F)c2)c2oc(-c3ccccc3)c(C)c(=O)c2c1] | 465 [M + H]⁺ |
| 178C | 8-[(1R)-1-[6-Chloro- 2-(7-oxa-2- azaspiro[3.5]nonan-2- yl)-3- pyridyl]amino]ethyl]- 3,6-dimethyl-2-(3- pyridyl)chromen-4-one | 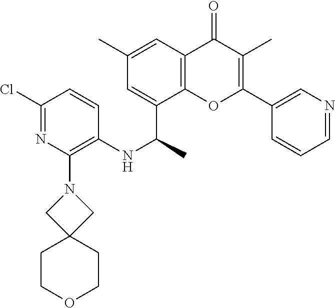 [SMILES: Cc1cc([C@@H](C)Nc2ccc(Cl)nc2N2CC3(CCOCC3)C2)c2oc(-c3cccnc3)c(C)c(=O)c2c1] | 531 [M + H]⁺ |
| 179C | 1-[6-Chloro-3-[[(1R)- 1-[3,6-dimethyl-4-oxo- 2-(3-pyridyl)chromen- 8-yl]ethyl]amino]-2- pyridyl]azetidine-3- carbonitrile | 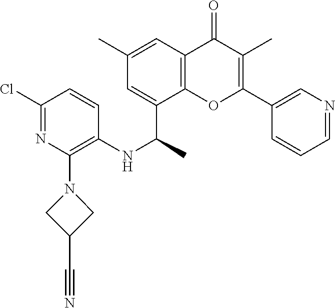 [SMILES: Cc1cc([C@@H](C)Nc2ccc(Cl)nc2N2CC(C#N)C2)c2oc(-c3cccnc3)c(C)c(=O)c2c1] | 486 [M + H]⁺ |
| 180C | 8-[(1R)-1-[[6-Chloro- 2-[4-(oxetan-3- yl)piperazin-1-y1]-3- pyridyl]amino]ethyl]- 3,6-dimethyl-2-(3- pyridyl)chromen-4-one | 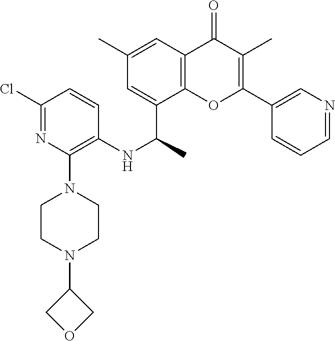 [SMILES: Cc1cc([C@@H](C)Nc2ccc(Cl)nc2N2CCN(C3COC3)CC2)c2oc(-c3cccnc3)c(C)c(=O)c2c1] | 546 [M + H]⁺ |
| 181C | 8-[(1R)-1-[[6-Chloro- 2-(1,4-oxazepan-4-y1)- 3-pyridyl]amino]ethyl]- 3,6-dimethyl-2-(3- pyridyl)chromen-4-one | 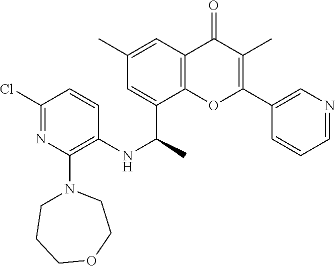 [SMILES: Cc1cc([C@@H](C)Nc2ccc(Cl)nc2N2CCCOCC2)c2oc(-c3cccnc3)c(C)c(=O)c2c1] | 505 [M + H]⁺ |
| 182C | 1-[6-Chloro-3-[[(1R)- 1-[3,6-dimethyl-4-oxo- 2-(3-pyridyl)chromen- 8-yl]ethyl]amino]-2- pyridyl]piperidine-4- carbonitrile | 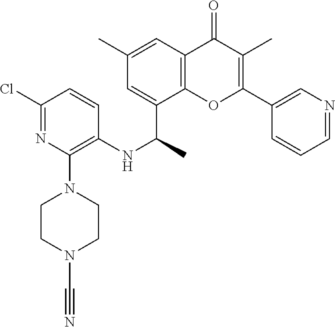 [SMILES: Cc1cc([C@@H](C)Nc2ccc(Cl)nc2N2CCN(C#N)CC2)c2oc(-c3cccnc3)c(C)c(=O)c2c1] | 514 [M + H]⁺ |

¹Iodinated pyridine reagent used in this coupling reaction.
²tBuXPhos Pd G3 used as the Pd catalyst and NaOtBu used as the base in this reaction.
³tBuBrettPhos Pd G3 used as the Pd catalyst and NaOtBu used as the base in this reaction.
⁴XantPhos Pd G4 used instead of Pd2(dba)3 and XantPhos.

### Example 11B: 8-[(1R)-1-(4-Chloro-3-fluoro-anilino)ethyl]-3,6-dimethyl-2-phenyl-chromen-4-one

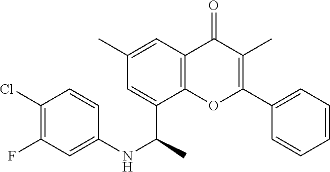 [SMILES: Cc1cc([C@@H](C)Nc2ccc(Cl)c(F)c2)c2oc(-c3ccccc3)c(C)c(=O)c2c1]

A mixture of di-mu-chlorobis[(1,2,3-nu)-1-phenyl-2-propen-1-yl]dipalladium (0.028 g, 0.051 mmol), 5-di-tert-butylphosphino-1′,3′,5′-triphenyl-1′H-1,4′-bipyrazole (0.10 g, 0.21 mmol), 8-[(1R)-1-aminoethyl]-3,6-dimethyl-2-phenyl-chromen-4-one (0.10 g, 0.34 mmol), 4-bromo-1-chloro-2-fluorobenzene (0.11 g, 0.51 mmol), and sodium tert-butoxide (0.046 g, 0.48 mmol) in toluene (2 mL) was heated to 100° C. for 14 h. The mixture was filtered over a pad of diatomaceous earth and the solids were rinsed with DCM. The filtrate was concentrated and the residue was purified by reverse phase chromatography eluted with a gradient of 10 to 100% ACN in aqueous NH₄HCO₃ (10 mM, plus 5% MeOH) to give the title compound (0.050 g, 35%). MS ES+ m/z 422 [M+H]⁺.

The following compounds in Table 93 were made in a similar way as described for 8-[(1R)-1-(4-chloro-3-fluoro-anilino)ethyl]-3,6-dimethyl-2-phenyl-chromen-4-one. Various methods were used to purify the compounds, which would be apparent to one skilled in the art.

TABLE 93

| Example | | | MS ES+ |
|---|---|---|---|
| # | Chemical Name | Structure | m/z |
| 12B | 8-[(1R)-1-(4- Chloroanilino)ethyl]- 3,6-dimethyl-2-phenyl- chromen-4-one | 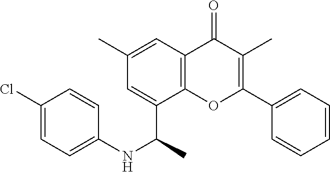 [SMILES: Cc1cc([C@@H](C)Nc2ccc(Cl)cc2)c2oc(-c3ccccc3)c(C)c(=O)c2c1] | 404 [M + H]⁺ |
| 13B | 3,6-Dimethyl-2-phenyl- 8-[(1R)-1-(3- pyridylamino)ethyl]chr omen-4-one | 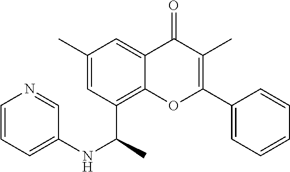 [SMILES: Cc1cc([C@@H](C)Nc2cccnc2)c2oc(-c3ccccc3)c(C)c(=O)c2c1] | 371 [M + H]⁺ |
| 14B | 8-[(1R)-1-[(6-Chloro- 3-pyridyl)amino]ethyl]- 3,6-dimethyl-2-phenyl- chromen-4-one | 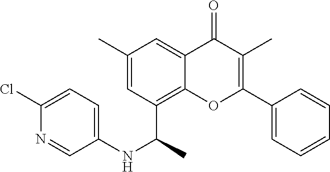 [SMILES: Cc1cc([C@@H](C)Nc2ccc(Cl)nc2)c2oc(-c3ccccc3)c(C)c(=O)c2c1] | 405 [M + H]⁺ |
| 15B | 3,6-Dimethyl-8-[(1R)- 1-[(6-methyl-3- pyridyl)amino]ethyl]-2- phenyl-chromen-4-one | 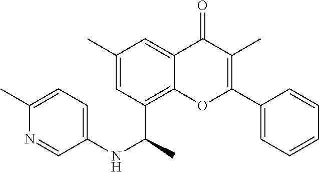 [SMILES: Cc1cc([C@@H](C)Nc2ccc(C)nc2)c2oc(-c3ccccc3)c(C)c(=O)c2c1] | 385 [M + H]⁺ |

### Example 16B: Methyl 3-[8-[(1R)-1-[(6-chloro-3-pyridyl)amino]ethyl]-3,6-dimethyl-4-oxo-chromen-2-yl]benzoate

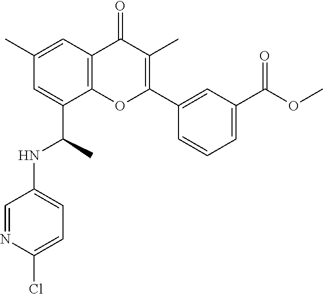 [SMILES: COC(=O)c1cccc(-c2oc3c([C@@H](C)Nc4ccc(Cl)nc4)cc(C)cc3c(=O)c2C)c1]

Combined 8-[(1R)-1-[(6-chloro-3-pyridyl)amino]ethyl]-2-ethylsulfanyl-3,6-dimethyl-chromen-4-one (0.10 g, 0.26 mmol), (3-(methoxycarbonyl)phenyl)boronic acid (0.14 g, 0.77 mmol), copper(I) thiophene-2-carboxylate (0.074 g, 0.39 mmol), and tetrakis(triphenylphosphine)palladium(O) (0.030 g, 0.026 mmol) in EtOH (3 mL). Stirred the reaction at 80° C. overnight. The mixture was filtered. The filtrate was concentrated and purified by silica gel chromatography eluted with a gradient of 0 to 100% EtOAc in hexanes to give the title compound (0.095 g, 80%). MS ES+ m/z 463 [M+H]⁺.

The following compounds in Table 94 were made in a similar way as described for methyl 3-[8-[(1R)-1-[(6-chloro-3-pyridyl)amino]ethyl]-3,6-dimethyl-4-oxo-chromen-2-yl]benzoate. Various methods were used to purify the compounds, which would be apparent to one skilled in the art.

TABLE 94

| Example | | | MS ES+ |
|---|---|---|---|
| # | Chemical Name | Structure | m/z |
| 17B | 8-[(1R)-1-[(6-Chloro- 3-pyridyl)amino]ethyl]- 3,6-dimethyl-2-(3- pyridyl)chromen-4-one | 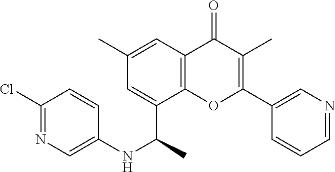 [SMILES: Cc1cc([C@@H](C)Nc2ccc(Cl)nc2)c2oc(-c3cccnc3)c(C)c(=O)c2c1] | 406 [M + H]⁺ |
| 18B | 8-[(1R)-1-[(6-Chloro- 3-pyridyl)amino]ethyl]- 3,6-dimethyl-2-(2- methylindazol-5- yl)chromen-4-one | 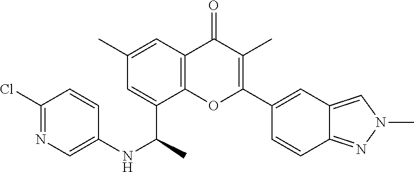 [SMILES: Cc1cc([C@@H](C)Nc2ccc(Cl)nc2)c2oc(-c3ccc4nn(C)cc4c3)c(C)c(=O)c2c1] | 459 [M + H]⁺ |
| 19B | 3-[8-[(1R)-1-[(6- Chloro-3- pyridyl)amino]ethyl]- 3,6-dimethyl-4-oxo- chromen-2- yl]benzonitrile | 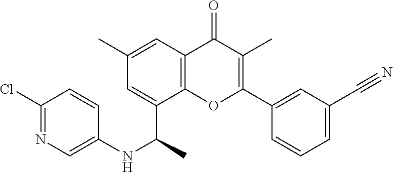 [SMILES: Cc1cc([C@@H](C)Nc2ccc(Cl)nc2)c2oc(-c3cccc(C#N)c3)c(C)c(=O)c2c1] | 430 [M + H]⁺ |
| 20B | Methyl 2-[3-[8-[(1R)- 1-[(6-chloro-3- pyridyl)amino]ethyl]- 3,6-dimethyl-4-oxo- chromen-2-y1]-2- fluoro-phenyl]acetate | 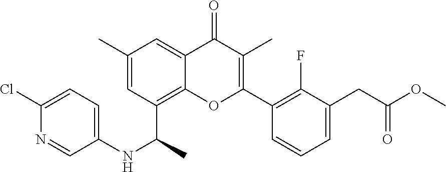 [SMILES: COC(=O)Cc1cccc(-c2oc3c([C@@H](C)Nc4ccc(Cl)nc4)cc(C)cc3c(=O)c2C)c1F] | 495 [M + H]⁺ |
| 21B | 8-[(1R)-1-[(6-Chloro- 3-pyridyl)amino]ethyl]- 2-[3- (dimethylamino)phenyl ]-3,6-dimethyl- chromen-4-one | 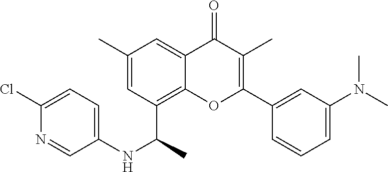 [SMILES: Cc1cc([C@@H](C)Nc2ccc(Cl)nc2)c2oc(-c3cccc(N(C)C)c3)c(C)c(=O)c2c1] | 448 [M + H]⁺ |
| 22B | 8-[(1R)-1-[(6-Chloro- 3-pyridyl)amino]ethyl]- 2-[4- [(dimethylamino) methy1]phenyl]-3,6- dimethyl-chromen- 4-one | 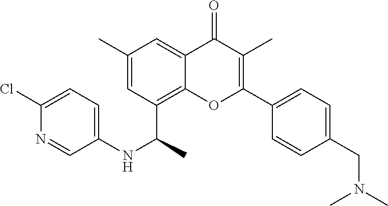 [SMILES: Cc1cc([C@@H](C)Nc2ccc(Cl)nc2)c2oc(-c3ccc(CN(C)C)cc3)c(C)c(=O)c2c1] | 462 [M + H]⁺ |
| 23B | 8-[(1R)-1-[(6-Chloro- 3-pyridyl)amino]ethyl]- 2-[3- [(dimethylamino)methy 1]phenyl]-3,6-dimethyl- chromen-4-one | 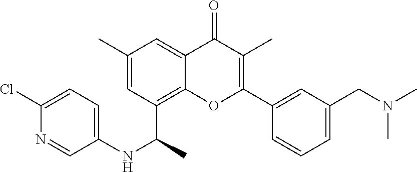 [SMILES: Cc1cc([C@@H](C)Nc2ccc(Cl)nc2)c2oc(-c3cccc(CN(C)C)c3)c(C)c(=O)c2c1] | 462 [M + H]⁺ |
| 24B | 8-[(1R)-1-[(6-Chloro- 3-pyridyl)amino]ethyl]- 2-[4- (dimethylamino)phenyl ]-3,6-dimethyl- chromen-4-one | 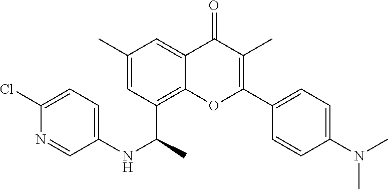 [SMILES: Cc1cc([C@@H](C)Nc2ccc(Cl)nc2)c2oc(-c3ccc(N(C)C)cc3)c(C)c(=O)c2c1] | 448 [M + H]⁺ |
| 25B | 8-[(1R)-1-[(6-Chloro- 3-pyridyl)amino]ethyl]- 3,6-dimethyl-2-[4-(3- pyridyl)phenyl] chromen-4-one 2,2,2- trifluoroacetic acid | 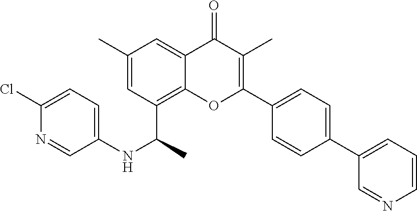 [SMILES: Cc1cc([C@@H](C)Nc2ccc(Cl)nc2)c2oc(-c3ccc(-c4cccnc4)cc3)c(C)c(=O)c2c1] | 482 [M + H]⁺ |
| | | 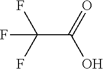 | |
| 26B | 3,6-Dimethyl-2-(2- methylindazol-5-y1)-8- [(1R)-1-[(2-methyl-3- pyridyl)amino]ethyl] chromen-4-one | 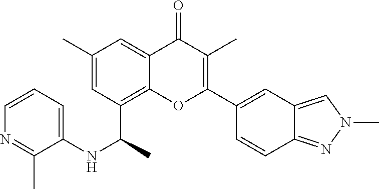 [SMILES: Cc1cc([C@@H](C)Nc2cccnc2C)c2oc(-c3ccc4nn(C)cc4c3)c(C)c(=O)c2c1] | 439 [M + H]⁺ |
| 46B¹ | 8-[(1R)-1-[(6-Chloro- 2-methyl-3- pyridyl)amino]ethyl]- 3,6-dimethyl-2-(1- methylpyrazol-3- yl)chromen-4-one | 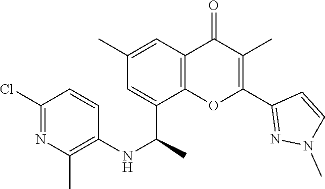 [SMILES: Cc1cc([C@@H](C)Nc2ccc(Cl)nc2C)c2oc(-c3ccn(C)n3)c(C)c(=O)c2c1] | 423 [M + H]⁺ |
| 47B | 8-[(1R)-1-[(6-Chloro- 2-methyl-3- pyridyl)amino]ethyl]-2- isothiazol-4-y1-3,6- dimethyl-chromen-4- one | 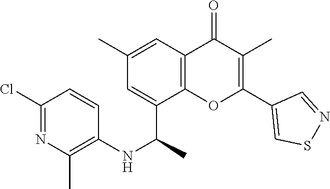 [SMILES: Cc1cc([C@@H](C)Nc2ccc(Cl)nc2C)c2oc(-c3cnsc3)c(C)c(=O)c2c1] | 426 [M + H]⁺ |
| 48B | 8-[(1R)-1-[(6-Chloro- 2-methyl-3- pyridyl)amino]ethyl]- 3,6-dimethyl-2-(1- methylindazol-5- yl)chromen-4-one | 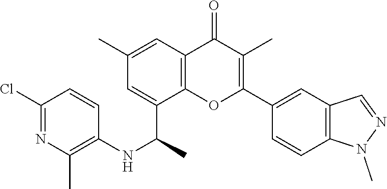 [SMILES: Cc1cc([C@@H](C)Nc2ccc(Cl)nc2C)c2oc(-c3ccc4c(cnn4C)c3)c(C)c(=O)c2c1] | 473 [M + H]⁺ |
| 49B | 8-[(1R)-1-[(6-Chloro- 2-methyl-3- pyridyl)amino]ethyl]-2- [1- (difluoromethyl)pyrazo 1-4-y1]-3,6-dimethyl- chromen-4-one | 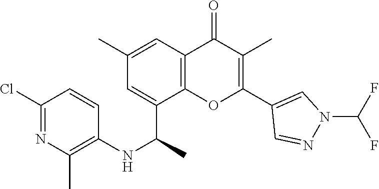 [SMILES: Cc1cc([C@@H](C)Nc2ccc(Cl)nc2C)c2oc(-c3cnn(C(F)F)c3)c(C)c(=O)c2c1] | 459 [M + H]⁺ |
| 50B | 8-[(1R)-1-[(2-Chloro- 3-pyridyl)amino]ethyl]- 3,6-dimethyl-2-(2- methylindazol-5- yl)chromen-4-one | 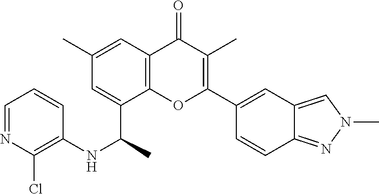 [SMILES: Cc1cc([C@@H](C)Nc2cccnc2Cl)c2oc(-c3ccc4nn(C)cc4c3)c(C)c(=O)c2c1] | 459 [M + H]⁺ |
| 51B | 3-[8-[(1R)-1-[(6- Chloro-2-methyl-3- pyridyl)amino]ethyl]- 3,6-dimethyl-4-oxo- chromen-2- yl]benzonitrile | 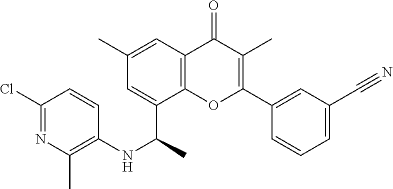 [SMILES: Cc1cc([C@@H](C)Nc2ccc(Cl)nc2C)c2oc(-c3cccc(C#N)c3)c(C)c(=O)c2c1] | 444 [M + H]⁺ |
| 52B | 8-[(1R)-1-[(6-Chloro- 2-methyl-3- pyridyl)amino]ethyl]- 3,6-dimethyl-2-(2- methyl-1,3- benzothiazol-6- yl)chromen-4-one | 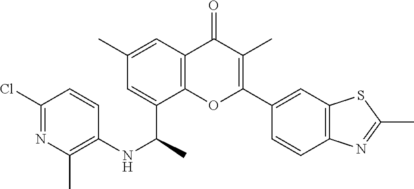 [SMILES: Cc1cc([C@@H](C)Nc2ccc(Cl)nc2C)c2oc(-c3ccc4nc(C)sc4c3)c(C)c(=O)c2c1] | 490 [M + H]⁺ |
| 53B | 8-[(1R)-1-[(6-Chloro- 2-methyl-3- pyridyl)amino]ethyl]- 3,6-dimethyl-2-phenyl- chromen-4-one | 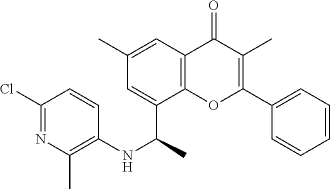 [SMILES: Cc1cc([C@@H](C)Nc2ccc(Cl)nc2C)c2oc(-c3ccccc3)c(C)c(=O)c2c1] | 419 [M + H]⁺ |
| 54B | 5-[8-[(1R)-1-[(6- Chloro-3- pyridyl)amino]ethyl]- 3,6-dimethyl-4-oxo- chromen-2-y1]-1,3- dihydrobenzimidazol- 2-one | 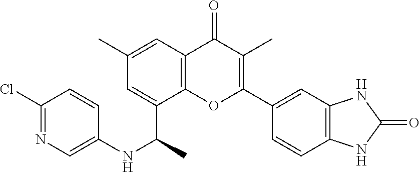 [SMILES: Cc1cc([C@@H](C)Nc2ccc(Cl)nc2)c2oc(-c3ccc4[nH]c(=O)[nH]c4c3)c(C)c(=O)c2c1] | 461 [M + H]⁺ |
| 55B | 6-[8-[(1R)-1-[(6- Chloro-3- pyridyl)amino]ethyl]- 3,6-dimethyl-4-oxo- chromen-2-y1]-3- methyl-1H- benzimidazol-2-one | 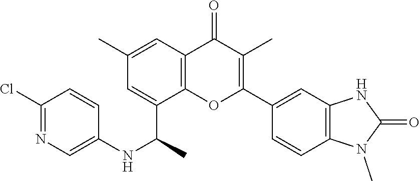 [SMILES: Cc1cc([C@@H](C)Nc2ccc(Cl)nc2)c2oc(-c3ccc4c(c3)[nH]c(=O)n4C)c(C)c(=O)c2c1] | 475 [M + H]⁺ |
| 56B | 8-[(1R)-1-[(2-Chloro- 3-pyridyl)amino]ethyl]- 3,6-dimethyl-2-(3- pyridyl)chromen-4-one | 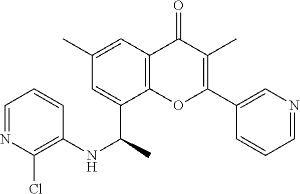 [SMILES: Cc1cc([C@@H](C)Nc2cccnc2Cl)c2oc(-c3cccnc3)c(C)c(=O)c2c1] | 406 [M + H]⁺ |
| 57B | 8-[(1R)-1-(3,4- Difluoro-2-methyl- anilino)ethyl]-3,6- dimethyl-2-(3- pyridyl)chromen-4-one | 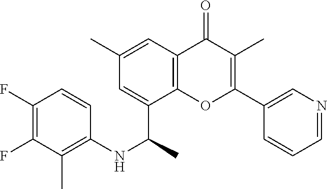 [SMILES: Cc1cc([C@@H](C)Nc2ccc(F)c(F)c2C)c2oc(-c3cccnc3)c(C)c(=O)c2c1] | 421 [M + H]⁺ |
| 58B | 3-[3,6-Dimethyl-8- [(1R)-1-[(2-methyl-3- pyridyl)amino]ethyl]-4- oxo-chromen-2- yl]benzonitrile | 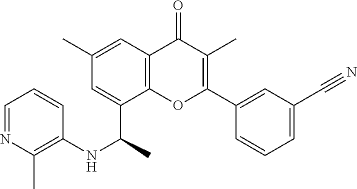 [SMILES: Cc1cc([C@@H](C)Nc2cccnc2C)c2oc(-c3cccc(C#N)c3)c(C)c(=O)c2c1] | 410 [M + H]⁺ |
| 59B | 3,6-Dimethyl-8-[(1R)- 1-[2-methyl-6- (trifluoromethyl)-3- pyridyl]amino]ethyl]-2- (3-pyridyl)chromen-4- one | 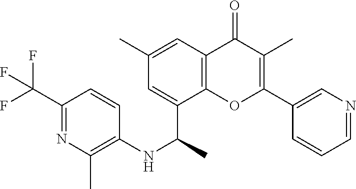 [SMILES: Cc1cc([C@@H](C)Nc2ccc(C(F)(F)F)nc2C)c2oc(-c3cccnc3)c(C)c(=O)c2c1] | 454 [M + H]⁺ |
| 60B | 3,6-Dimethyl-2-(1- methylpyrazol-4-yl)-8- [(1R)-1-[[2-methyl-6- (trifluoromethyl)-3- pyridyl]amino]ethyl]ch romen-4-one | 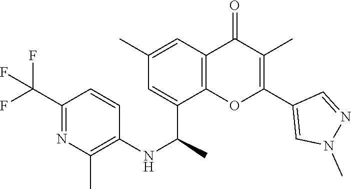 [SMILES: Cc1cc([C@@H](C)Nc2ccc(C(F)(F)F)nc2C)c2oc(-c3cnn(C)c3)c(C)c(=O)c2c1] | 457 [M + H]⁺ |
| 61B | 5-[8-[(1R)-1-[(6- Chloro-2-methyl-3- pyridyl)amino]ethyl]- 3,6-dimethyl-4-oxo- chromen-2-y1]-1H- pyridin-2-one | 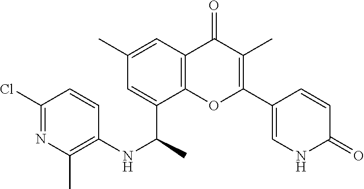 [SMILES: Cc1cc([C@@H](C)Nc2ccc(Cl)nc2C)c2oc(-c3ccc(=O)[nH]c3)c(C)c(=O)c2c1] | 436 [M + H]⁺ |
| 62B | 4-[8-[(1R)-1-[(6- Chloro-2-methyl-3- pyridyl)amino]ethyl]- 3,6-dimethyl-4-oxo- chromen-2-y1]-1H- pyridin-2-one | 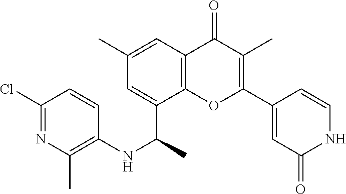 [SMILES: Cc1cc([C@@H](C)Nc2ccc(Cl)nc2C)c2oc(-c3cc[nH]c(=O)c3)c(C)c(=O)c2c1] | 436 [M + H]⁺ |
| 63B | 8-[(1R)-1-[(6-Chloro- 2-methyl-3- pyridyl)amino]ethyl]-2- [1-(1,1-dioxothietan-3- yl)pyrazol-4- y1]64B3,6-dimethyl- chromen-4-one | 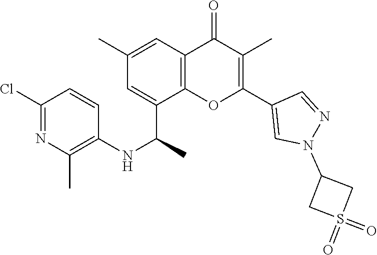 [SMILES: Cc1cc([C@@H](C)Nc2ccc(Cl)nc2C)c2oc(-c3cnn(C4CS(=O)(=O)C4)c3)c(C)c(=O)c2c1] | 513 [M + H]⁺ |
| 64B | 8-[(1R)-1-[(6-Chloro- 2-methyl-3- pyridyl)amino]ethyl]- 3,6-dimethy1-2-[1- (oxetan-3-yl)pyrazol-4- yl]chromen-4-one | 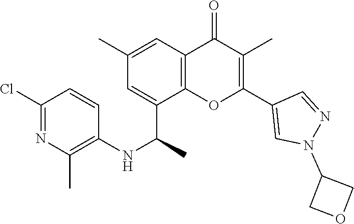 [SMILES: Cc1cc([C@@H](C)Nc2ccc(Cl)nc2C)c2oc(-c3cnn(C4COC4)c3)c(C)c(=O)c2c1] | 465 [M + H]⁺ |
| 65B | 8-[(1R)-1-[(6-Chloro- 2-methyl-3- pyridyl)amino]ethyl]-2- (1H-indol-6-yl)-3,6- dimethyl-chromen-4- one | 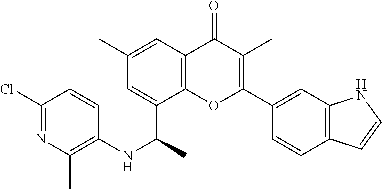 [SMILES: Cc1cc([C@@H](C)Nc2ccc(Cl)nc2C)c2oc(-c3ccc4cc[nH]c4c3)c(C)c(=O)c2c1] | 458 [M + H]⁺ |
| 66B | 8-[(1R)-1-[(2,6- Dimethy1-3- pyridyl)amino]ethyl]- 3,6-dimethyl-2-(2- methylindazol-5- yl)chromen-4-one | 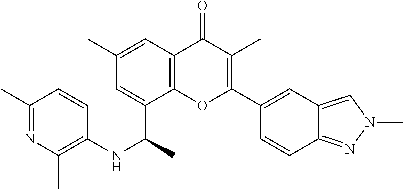 | 453 [M + H]⁺ |
| 67B | 8-[(1R)-1-[(6-Chloro- 2-methyl-3- pyridyl)amino]ethyl]-2- [1-(2-hydroxy-2- methyl-propyl)pyrazol- 4-y1]-3,6-dimethyl- chromen-4-one | 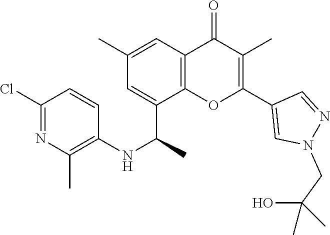 [SMILES: Cc1cc([C@@H](C)Nc2ccc(Cl)nc2C)c2oc(-c3cnn(CC(C)(C)O)c3)c(C)c(=O)c2c1] | 481 [M + H]⁺ |
| 68B | 8-[(1R)-1-[(6-Chloro- 2-methyl-3- pyridyl)amino]ethyl]- 3,6-dimethyl-2-(1- methylpyrazolo[3,4- b]pyridin-6- yl)chromen-4-one | 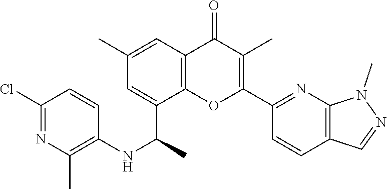 [SMILES: Cc1cc([C@@H](C)Nc2ccc(Cl)nc2C)c2oc(-c3ccc4cnn(C)c4n3)c(C)c(=O)c2c1] | 474 [M + H]⁺ |
| 69B | 8-[(1R)-1-[(6-Chloro- 3-pyridyl)amino]ethyl]- 2-(1H-indol-6-yl)-3,6- dimethyl-chromen-4- one | 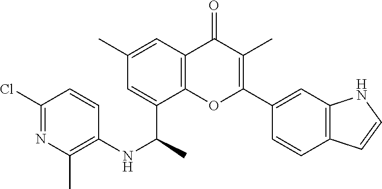 | 444 [M + H]⁺ |
| 70B | 8-[(1R)-1-[(6- Methoxy-2-methyl-3- pyridyl)amino]ethyl]- 3,6-dimethyl-2-(3- pyridyl)chromen-4-one | 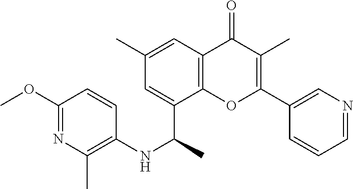 [SMILES: COc1ccc(N[C@H](C)c2cc(C)cc3c(=O)c(C)c(-c4cccnc4)oc23)c(C)n1] | 416 [M + H]⁺ |
| 71B | 6-[8-[(1R)-1-[(6- Chloro-2-methyl-3- pyridyl)amino]ethyl]- 3,6-dimethyl-4-oxo- chromen-2-y1]-1H- pyridin-2-one | 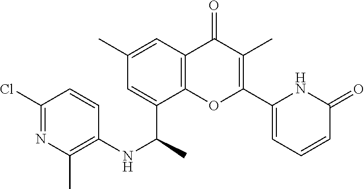 [SMILES: Cc1cc([C@@H](C)Nc2ccc(Cl)nc2C)c2oc(-c3cccc(=O)[nH]3)c(C)c(=O)c2c1] | 436 [M + H]⁺ |
| 183C | 4-[8-[(1R)-1-[2-(2- Fluorophenyl)-3- pyridyl]amino]ethyl]- 3,6-dimethyl-4-oxo- chromen-2-y1]-1H- pyridin-2-one | 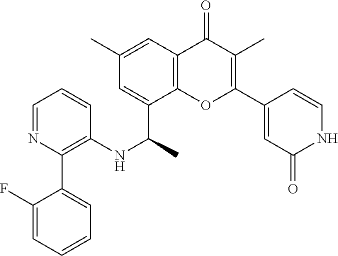 [SMILES: Cc1cc([C@@H](C)Nc2cccnc2-c2ccccc2F)c2oc(-c3cc[nH]c(=O)c3)c(C)c(=O)c2c1] | 482 [M + H]⁺ |
| 184C | 5-[8-[(1R)-1-[2-(2- Fluorophenyl)-3- pyridyl]amino]ethyl]- 3,6-dimethyl-4-oxo- chromen-2-y1]-1- methyl-pyridin-2-one | 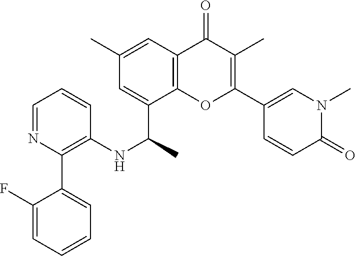 [SMILES: Cc1cc([C@@H](C)Nc2cccnc2-c2ccccc2F)c2oc(-c3ccc(=O)n(C)c3)c(C)c(=O)c2c1] | 496 [M + H]⁺ |
| 185C | 5-[8-[(1R)-1-[2-(2- Fluorophenyl)-3- pyridyl]amino]ethyl]- 3,6-dimethyl-4-oxo- chromen-2-y1]-1H- pyridin-2-one | 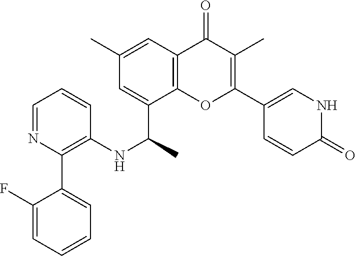 [SMILES: Cc1cc([C@@H](C)Nc2cccnc2-c2ccccc2F)c2oc(-c3ccc(=O)[nH]c3)c(C)c(=O)c2c1] | 482 [M + H]⁺ |
| 186C | 8-[(1R)-1-[(6-Chloro- 2-methyl-3- pyridyl)amino]ethyl]-2- (1,5-dimethylpyrazol- 4-y1)-3,6-dimethyl- chromen-4-one | 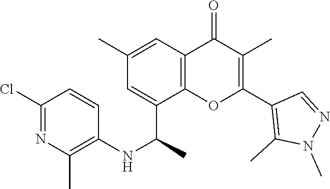 [SMILES: Cc1cc([C@@H](C)Nc2ccc(Cl)nc2C)c2oc(-c3cnn(C)c3C)c(C)c(=O)c2c1] | 437 [M + H]⁺ |
| 187C | 8-[(1R)-1-[(6-Chloro- 2-methyl-3- pyridyl)amino]ethyl]-2- (1,3-dimethylpyrazol- 4-y1)-3,6-dimethyl- chromen-4-one | 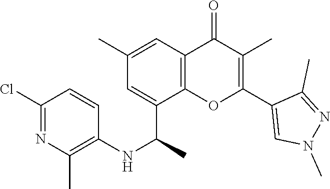 [SMILES: Cc1cc([C@@H](C)Nc2ccc(Cl)nc2C)c2oc(-c3cn(C)nc3C)c(C)c(=O)c2c1] | 437 [M + H]⁺ |
| 188C | 8-[(1R)-1-[(6-Chloro- 2-methyl-3- pyridyl)amino]ethyl]- 3,6-dimethyl-2-(1- methyltriazol-4- yl)chromen-4-one | 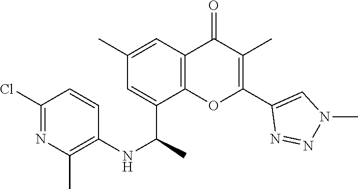 [SMILES: Cc1cc([C@@H](C)Nc2ccc(Cl)nc2C)c2oc(-c3cn(C)nn3)c(C)c(=O)c2c1] | 424 [M + H]⁺ |
| 189C¹ | 8-[(1R)-1-[(6-Chloro- 2-methyl-3- pyridyl)amino]ethyl]- 3,6-dimethyl-2-(1- methylbenzotriazol-5- yl)chromen-4-one | 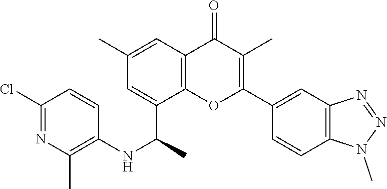 [SMILES: Cc1cc([C@@H](C)Nc2ccc(Cl)nc2C)c2oc(-c3ccc4c(c3)nnn4C)c(C)c(=O)c2c1] | 474 [M + H]⁺ |
| 190C | 2-(2-Fluorophenyl)-8- [(1R)-1-[2-(2- fluorophenyl)-3- pyridyl]amino]ethyl]- 3,6-dimethyl-chromen- 4-one | 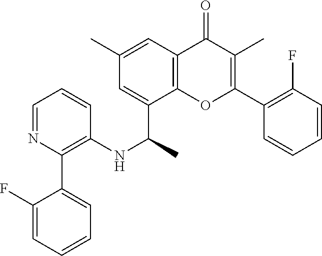 [SMILES: Cc1cc([C@@H](C)Nc2cccnc2-c2ccccc2F)c2oc(-c3ccccc3F)c(C)c(=O)c2c1] | 483 [M + H]⁺ |
| 191C | 8-[(1R)-1-[[2-(2- Fluorophenyl)-3- pyridyl]amino]ethyl]- 3,6-dimethyl-2-(2- methylindazol-5- yl)chromen-4-one | 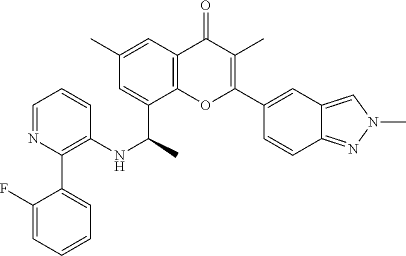 [SMILES: Cc1cc([C@@H](C)Nc2cccnc2-c2ccccc2F)c2oc(-c3ccc4nn(C)cc4c3)c(C)c(=O)c2c1] | 519 |
| 192C | 8-[(1R)-1-[[2-(2- Fluorophenyl)-3- pyridyl]amino]ethyl]- 3,6-dimethyl-2-(3- pyridyl)chromen-4-one | 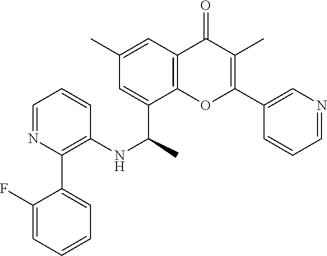 [SMILES: Cc1cc([C@@H](C)Nc2cccnc2-c2ccccc2F)c2oc(-c3cccnc3)c(C)c(=O)c2c1] | 466 [M + H]⁺ |
| 193C | 8-[(1R)-1-[2-(2- Fluorophenyl)-3- pyridyl]amino]ethyl]- 3,6-dimethyl-2-(1- methylpyrazol-4- yl)chromen-4-one | 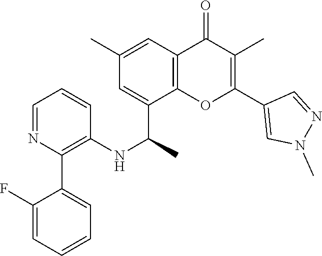 [SMILES: Cc1cc([C@@H](C)Nc2cccnc2-c2ccccc2F)c2oc(-c3cnn(C)c3)c(C)c(=O)c2c1] | 469 [M + H]⁺ |
| 194C | 8-[(1R)-1-[[2-(2- Fluorophenyl)-3- pyridyl]amino]ethyl]- 3,6-dimethyl-2-(2- pyridyl)chromen-4-one | 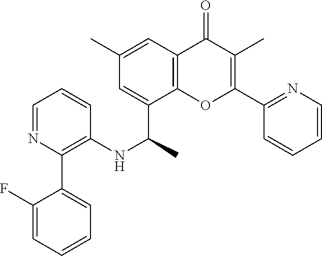 [SMILES: Cc1cc([C@@H](C)Nc2cccnc2-c2ccccc2F)c2oc(-c3ccccn3)c(C)c(=O)c2c1] | 466 [M + H]⁺ |
| 195C | 8-[(1R)-1-[2-(2- Fluorophenyl)-3- pyridyl]amino]ethyl]- 3,6-dimethyl-2-(2- methylimidazo[1,2- a]pyridin-6- yl)chromen-4-one | 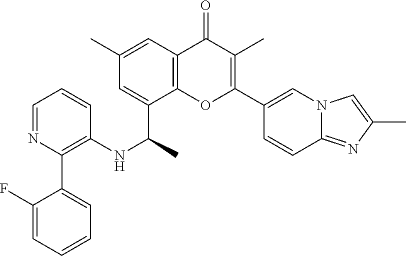 [SMILES: Cc1cc([C@@H](C)Nc2cccnc2-c2ccccc2F)c2oc(-c3ccc4nc(C)cn4c3)c(C)c(=O)c2c1] | 519 [M + H]⁺ |
| 196C | 8-[(1R)-1-[[2-(2- Fluorophenyl)-3- pyridyl]amino]ethyl]-2- (5-fluoro-3-pyridyl)- 3,6-dimethyl-chromen- 4-one | 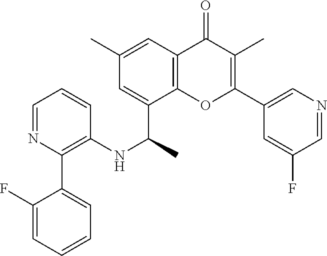 [SMILES: Cc1cc([C@@H](C)Nc2cccnc2-c2ccccc2F)c2oc(-c3cncc(F)c3)c(C)c(=O)c2c1] | 484 [M + H]⁺ |
| 197C | 8-[(1R)-1-[(6-Chloro- 2-methyl-3- pyridyl)amino]ethyl]- 3,6-dimethyl-2-(1- methylpyrazolo[4,3- b]pyridin-6- yl)chromen-4-one | 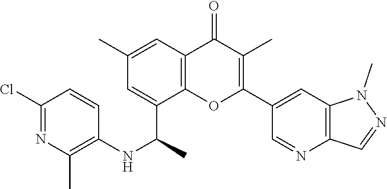 [SMILES: Cc1cc([C@@H](C)Nc2ccc(Cl)nc2C)c2oc(-c3cnc4cnn(C)c4c3)c(C)c(=O)c2c1] | 474 [M + H]⁺ |
| 198C | 8-[(1R)-1-[(6-Chloro- 2-methyl-3- pyridyl)amino]ethyl]- 3,6-dimethyl-2-(1- methylpyrazolo[4,3- b]pyridin-5- y1)chromen-4-one | 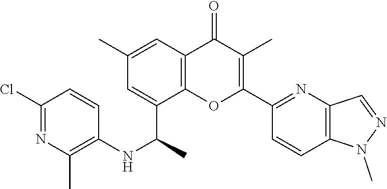 [SMILES: Cc1cc([C@@H](C)Nc2ccc(Cl)nc2C)c2oc(-c3ccc4c(cnn4C)n3)c(C)c(=O)c2c1] | 474 [M + H]⁺ |
| 199C | 8-[(1R)-1-[(6-Chloro- 2-methyl-3- pyridyl)amino]ethyl]- 3,6-dimethyl-2- pyrimidin-5-yl- chromen-4-one | 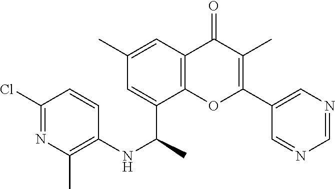 [SMILES: Cc1cc([C@@H](C)Nc2ccc(Cl)nc2C)c2oc(-c3cncnc3)c(C)c(=O)c2c1] | 421 [M + H]⁺ |
| 200C | 5-[8-[(1R)-1-[(6- Chloro-2-methyl-3- pyridyl)amino]ethyl]- 3,6-dimethyl-4-oxo- chromen-2-yl]pyridine- 3-carbonitrile | 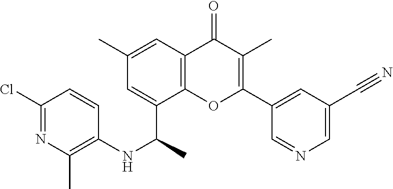 [SMILES: Cc1cc([C@@H](C)Nc2ccc(Cl)nc2C)c2oc(-c3cncc(C#N)c3)c(C)c(=O)c2c1] | 445 [M + H]⁺ |
| 201C | 3,6-Dimethyl-2-(2- methylindazol-5-y1)-8- [(1R)-1-[[6- (trifluoromethyl)-3- pyridyl]amino]ethyl]ch romen-4-one | 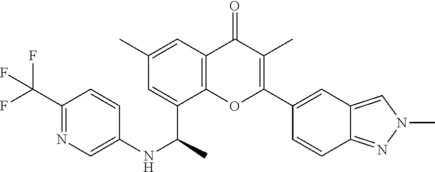 [SMILES: Cc1cc([C@@H](C)Nc2ccc(C(F)(F)F)nc2)c2oc(-c3ccc4nn(C)cc4c3)c(C)c(=O)c2c1] | 493 [M + H]⁺ |
| 202C | 6-[[(1R)-1-[3,6- Dimethyl-2-(1- methylpyrazol-4-y1)-4- oxo-chromen-8- yl]ethyl]amino]-2,3- difluoro-benzonitrile | 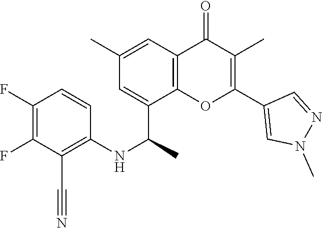 [SMILES: Cc1cc([C@@H](C)Nc2ccc(F)c(F)c2C#N)c2oc(-c3cnn(C)c3)c(C)c(=O)c2c1] | 435 [M + H]⁺ |
| 203C² | Methyl 3-[8-[(1R)-1- [[6-chloro-2- (methylsulfonylcarbam oy1)-3- pyridyl]amino]ethyl]- 3,6-dimethyl-4-oxo- chromen-2-yl]benzoate | 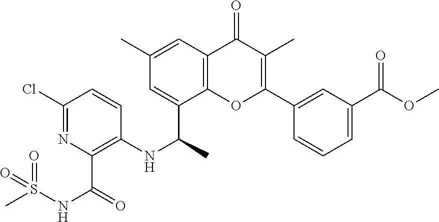 | 584 [M + H]⁺ |
| 204C² | 6-Chloro-3-[(1R)-1- [2-(2- cyclopropylpyrimidin- 5-y1)-3,6-dimethyl-4- oxo-chromen-8- yl]ethyl]amino]-N- methylsulfonyl- pyridine-2- carboxamide | 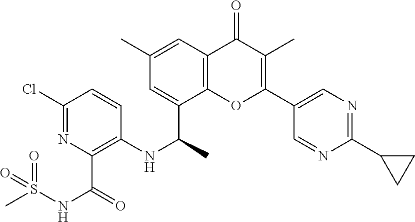 [SMILES: Cc1cc([C@@H](C)Nc2ccc(Cl)nc2C(=O)NS(C)(=O)=O)c2oc(-c3cnc(C4CC4)nc3)c(C)c(=O)c2c1] | 568 [M + H]⁺ |
| 205C² | 3-[(1R)-1-[2-(3- Acetamidophenyl)-3,6- dimethyl-4-oxo- chromen-8- yl]ethyl]amino]-6- chloro-N- methylsulfonyl- pyridine-2- carboxamide | 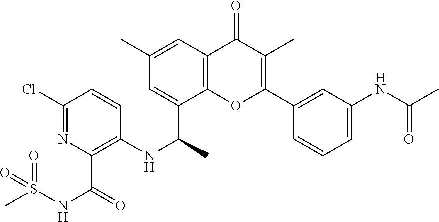 [SMILES: CC(=O)Nc1cccc(-c2oc3c([C@@H](C)Nc4ccc(Cl)nc4C(=O)NS(C)(=O)=O)cc(C)cc3c(=O)c2C)c1] | 583 [M + H]⁺ |
| 206C² | 6-Chloro-3-[[(1R)-1- [3,6-dimethyl-4-oxo-2- (1H-pyrazol-3- yl)chromen-8- yl]ethyl]amino]-N- methylsulfonyl- pyridine-2- carboxamide | 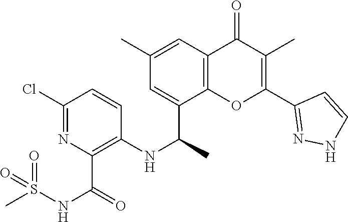 [SMILES: Cc1cc([C@@H](C)Nc2ccc(Cl)nc2C(=O)NS(C)(=O)=O)c2oc(-c3cc[nH]n3)c(C)c(=O)c2c1] | 516 [M + H]⁺ |
| 207C | 6-[8-[(1R)-1-[(6- Chloro-3- pyridyl)amino]ethyl]- 3,6-dimethyl-4-oxo- chromen-2-y1]-1H- indole-3-carboxylic acid | 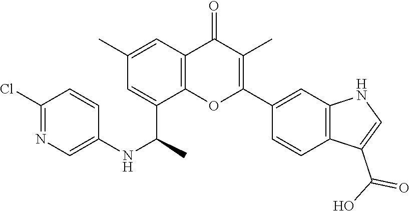 [SMILES: Cc1cc([C@@H](C)Nc2ccc(Cl)nc2)c2oc(-c3ccc4c(C(=O)O)c[nH]c4c3)c(C)c(=O)c2c1] | 488 [M + H]⁺ |
| 240C² | 6-Chloro-3-[(1R)-1- [2-[1-(2- hydroxyethyl)pyrazol- 4-y1]-3,6-dimethyl-4- oxo-chromen-8- yl]ethyl]amino]-N- methylsulfonyl- pyridine-2- carboxamide | 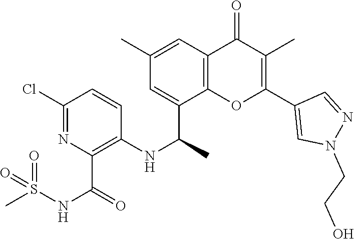 [SMILES: Cc1cc([C@@H](C)Nc2ccc(Cl)nc2C(=O)NS(C)(=O)=O)c2oc(-c3cnn(CCO)c3)c(C)c(=O)c2c1] | 560 [M]⁺ |
| 241C² | 6-Chloro-3-[[(1R)-1- [2-[1-(2-hydroxy-2- methyl-propyl)pyrazol- 4-yl]-3,6-dimethyl-4- oxo-chromen-8- yl]ethyl]amino]-N- methylsulfonyl- pyridine-2- carboxamide | 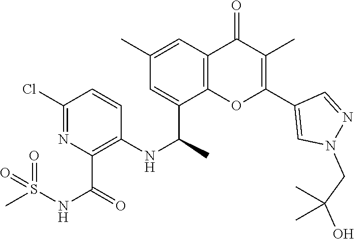 [SMILES: Cc1cc([C@@H](C)Nc2ccc(Cl)nc2C(=O)NS(C)(=O)=O)c2oc(-c3cnn(CC(C)(C)O)c3)c(C)c(=O)c2c1] | 588 [M]⁺ |
| 242C¹ | 6-Chloro-3-[(1R)-1- [3,6-dimethyl-2-(2- methylpyrazolo[3,4- c]570yridine-5-y1)-4- oxo-chromen-8- yl]ethyl]amino]-N- methylsulfonyl- pyridine-2- carboxamide | 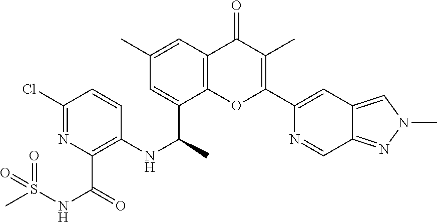 | 581 [M]⁺ |
| 243C² | 6-Chloro-3-[[(1R)-1- [3,6-dimethyl-4-oxo-2- (1-tetrahydropyran-4- ylpyrazol-4- yl)chromen-8- yl]ethyl]amino]-N- methylsulfonyl- pyridine-2- carboxamide | 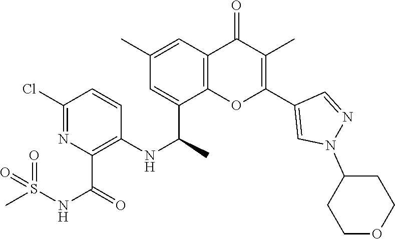 [SMILES: Cc1cc([C@@H](C)Nc2ccc(Cl)nc2C(=O)NS(C)(=O)=O)c2oc(-c3cnn(C4CCOCC4)c3)c(C)c(=O)c2c1] | 600 [M]⁺ |
| 244C² | 6-Chloro-3-[(1R)-1- [2-[1-(2- methoxypyrimidin-5- y1)pyrazol-4-y1]-3,6- dimethyl-4-oxo- chromen-8- yl]ethyl]amino]-N- methylsulfonyl- pyridine-2- carboxamide | 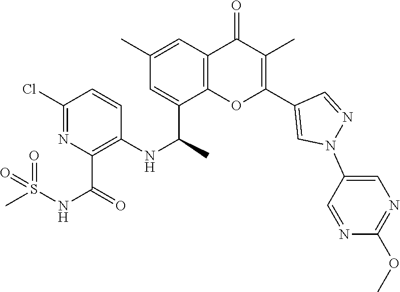 [SMILES: COc1ncc(-n2cc(-c3oc4c([C@@H](C)Nc5ccc(Cl)nc5C(=O)NS(C)(=O)=O)cc(C)cc4c(=O)c3C)cn2)cn1] | 624 [M]⁺ |

¹Cesium carbonate also used in this reaction.
²1,1'-Bis(diphenylphosphino)ferrocenedichloropalladium(II) used as catalyst and cesium carbonate also used in this reaction.

### Example 208C: 2-(2,6-Difluorophenyl)-8-[(1R)-1-[[2-(2-fluorophenyl)-3-pyridyl]amino]ethyl]-3,6-dimethyl-chromen-4-one

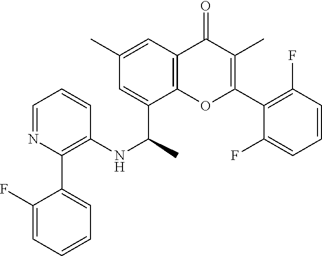 [SMILES: Cc1cc([C@@H](C)Nc2cccnc2-c2ccccc2F)c2oc(-c3c(F)cccc3F)c(C)c(=O)c2c1]

Combined 2-ethylsulfanyl-8-[(1R)-1-[[2-(2-fluorophenyl)-3-pyridyl]amino]ethyl]-3,6-dimethyl-chromen-4-one (250 mg, 0.56 mmol), copper(I) thiophene-2-carboxylate (159 mg, 0.84 mmol), and APhos Pd G3 (106 mg, 0.17 mmol) with THF (5.6 mL) in a vial. The reaction was sparged with argon for 5 min and then was treated with (2,6-difluorophenyl)zinc(II) bromide (0.5 M in THF, 720 mg, 2.79 mmol) dropwise. After addition, the vial was sealed and the reaction stirred at 80° C. for 16 h. The reaction was cooled to rt, filtered through diatomaceous earth, and the solids washed with DCM. The filtrate was concentrated under reduced pressure and the residue purified by reversed phase chromatography on C18 eluted with 0% to 60% ACN in water to give the title compound (166 mg, 57%) as an off-white foam. ES/MS m/z 501 (M+H).

### Example 209C: 8-[(1R)-1-[2-(6-Hydroxy-3-pyridyl)anilino]ethyl]-3,6-dimethyl-2-phenyl-chromen-4-one

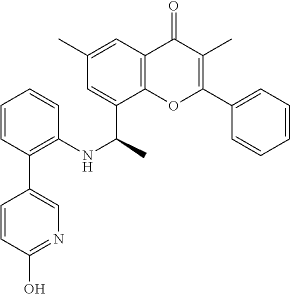 [SMILES: Cc1cc([C@@H](C)Nc2ccccc2-c2ccc(O)nc2)c2oc(-c3ccccc3)c(C)c(=O)c2c1]

Combined 8-[(1R)-1-(2-bromoanilino)ethyl]-3,6-dimethyl-2-phenyl-chromen-4-one (50 mg, 0.11 mmol), 5-(4,4,5,5-tetramethyl-1,3,2-dioxaborolan-2-yl)pyridin-2-ol (30 mg, 0.13 mmol), 1,1′-bis(diphenylphosphino)ferrocenedichloropalladium(II) dichloromethane complex (8.2 mg, 0.1 mmol), and potassium carbonate (46 mg, 0.33 mmol) in ACN (2 mL). Degassed with argon for 1 min and stirred at 95° C. for 16 h. The reaction was loaded directly onto a diatomaceous earth cartridge and purified by reversed phase chromatography on C18 eluted with 1000 to 100% ACN in 10 mM aqueous NH₄HCO₃ with 500 MeOH to give the title compound (9.3 mg, 18). ES/MS m/z 463 (M+H).

The following compounds in Table 95 were made in a similar way as described for 8-[(1R)-1-[2-(6-hydroxy-3-pyridyl)anilino]ethyl]-3,6-dimethyl-2-phenyl-chromen-4-one. Various methods were used to purify the compounds, which would be apparent to one skilled in the art.

TABLE 95

| | | | MS ES+ |
|---|---|---|---|
| Example # | Chemical Name | Structure | m/z |
| 210C | 4-[2-[[(1R)-1-(3,6- Dimethyl-4-oxo-2- phenyl-chromen-8- yl)ethyl]amino]phenyl]- 1H-pyridin-2-one | 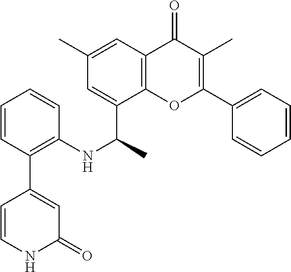 [SMILES: Cc1cc([C@@H](C)Nc2ccccc2-c2cc[nH]c(=O)c2)c2oc(-c3ccccc3)c(C)c(=O)c2c1] | 463 [M + H]⁺ |
| 211C¹ | 4-[2-[[(1R)-1-(3,6- Dimethyl-4-oxo-2- phenyl-chromen-8- yl)ethyl]amino]phenyl]- 1-methyl-pyridin-2- one | 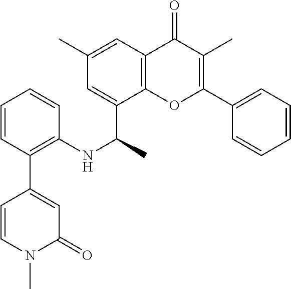 [SMILES: Cc1cc([C@@H](C)Nc2ccccc2-c2ccn(C)c(=O)c2)c2oc(-c3ccccc3)c(C)c(=O)c2c1] | 477 [M + H]⁺ |
| 212C² | 8-[(1R)-1-[[2-(2- Fluorophenyl)-3- pyridyl]amino]ethyl]- 3,6-dimethyl-2-phenyl- chromen-4-one | 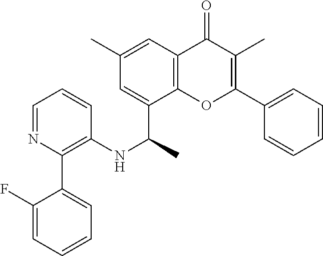 [SMILES: Cc1cc([C@@H](C)Nc2cccnc2-c2ccccc2F)c2oc(-c3ccccc3)c(C)c(=O)c2c1] | 465 [M + H]⁺ |

¹1,4-Dioxane and water used as the solvent.
²1,1′-Bis(di-tert-butylphosphino)ferrocene palladium dichloride used as catalyst and 1,4-dioxane used as a solvent.

### Example 72B: 8-[(1R)-1-[(6-Chloro-2-methoxy-3-pyridyl)amino]ethyl]-3,6-dimethyl-2-(3-pyridyl)chromen-4-one

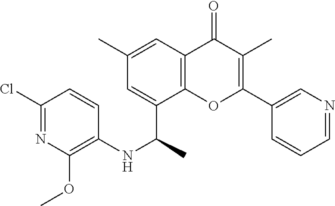 [SMILES: COc1nc(Cl)ccc1N[C@H](C)c1cc(C)cc2c(=O)c(C)c(-c3cccnc3)oc12]

A 20 mL vial was charged with 8-[(1R)-1-aminoethyl]-3,6-dimethyl-2-(3-pyridyl)chromen-4-one (75 mg, 0.25 mmol), 6-chloro-3-iodo-2-methoxypyridine (82 mg, 0.31 mmol), cesium carbonate (170 mg, 0.51 mmol), palladium(II) acetate (5.7 mg, 0.03 mmol), and BINAP (48 mg, 0.08 mmol), and 1,4-dioxane (5 mL). The reaction mixture was degassed with nitrogen gas for 5 minutes and the suspension stirred at 90° C. for 17 h. The reaction was passed through a syringe filter and the filtrate purified by reversed phase chromatography eluted with 10 to 100% ACN in 10 mM NH₄HCO₃ with 5% MeOH. The resulting material was purified by preparative HPLC eluted with 45 to 100% ACN in 10 mM NH₄HCO₃ with 5% MeOH to give the title compound (16.4 mg, 15%) as a white solid. MS ES+ m/z 436 [M+H]⁺.

The following compounds in Table 96 were made in a similar way as described for 8-[(1R)-1-[(6-chloro-2-methoxy-3-pyridyl)amino]ethyl]-3,6-dimethyl-2-(3-pyridyl)chromen-4-one. Various methods were used to purify the compound, which would be apparent to one skilled in the art.

TABLE 96

| | | | MS ES+ |
|---|---|---|---|
| Example # | Chemical Name | Structure | m/z |
| 73B¹ | 8-[(1R)-1-[(2,6- Dimethyl-3- pyridyl)amino]ethyl]- 3,6-dimethyl-2-(1- methylpyrazol-4- yl)chromen-4-one | 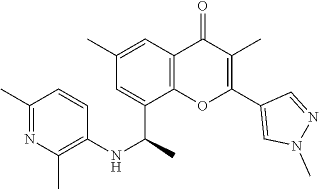 [SMILES: Cc1cc([C@@H](C)Nc2ccc(C)nc2C)c2oc(-c3cnn(C)c3)c(C)c(=O)c2c1] | 403 [M + H]⁺ |

### Example 74B: 4-[8-[(1R)-1-[(6-Chloro-2-methyl-3-pyridyl)amino]ethyl]-3,6-dimethyl-4-oxo-chromen-2-yl]-2-methyl-pyrazole-3-carboxamide

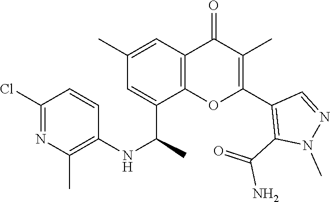 [SMILES: Cc1cc([C@@H](C)Nc2ccc(Cl)nc2C)c2oc(-c3cnn(C)c3C(N)=O)c(C)c(=O)c2c1]

8-[(1R)-1-[(6-Chloro-2-methyl-3-pyridyl)amino]ethyl]-2-ethylsulfanyl-3,6-dimethyl-chromen-4-one (100 mg, 0.25 mmol), 4-bromo-2-methyl-pyrazole-3-carboxamide (76 mg, 0.37 mmol), copper(I) thiophene-2-carboxylate (142 mg, 0.75 mmol), cesium carbonate (323 mg, 0.99 mmol), bis(pinacolato)diboron (252 mg, 0.99 mmol) RuPhos Pd G4 (21.1 mg, 0.03 mmol), XPhos Pd G4 (21.4 mg, 0.03 mmol) were combined in 1,4-dioxane (5 mL) and water (0.1 mL). The reaction was degassed with argon for 5 min and then the reaction was stirred at 100° C. for 16 h. The reaction was allowed to cool, filtered, concentrated, and purified by silica gel chromatography eluted with 0 to 20% MeOH in DCM. The residue was re-purified by reversed phase chromatography eluted with 0 to 100% ACN in 10 mM NH₄HCO₃ with 5% MeOH to give the title compound (2.5 mg, 2%). MS ES+ m/z 466 [M+H]⁺.

The following compound in Table 97 was made in a similar way as described for 4-[8-[(1R)-1-[(6-chloro-2-methyl-3-pyridyl)amino]ethyl]-3,6-dimethyl-4-oxo-chromen-2-yl]-2-methyl-pyrazole-3-carboxamide. Various methods were used to purify the compound, which would be apparent to one skilled in the art.

TABLE 97

| | | | MS ES+ |
|---|---|---|---|
| Example # | Chemical Name | Structure | m/z |
| 213C | 8-[(1R)-1-[(6-Chloro- 2-methyl-3- pyridyl)amino]ethyl]-2- (5-fluoro-1-methyl- pyrazol-4-yl)-3,6- dimethyl-chromen-4- one | 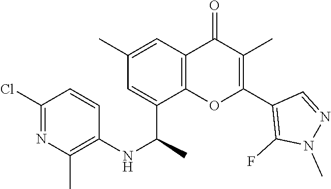 [SMILES: Cc1cc([C@@H](C)Nc2ccc(Cl)nc2C)c2oc(-c3cnn(C)c3F)c(C)c(=O)c2c1] | 441 [M + H]⁺ |

### Example 214C: 6-Chloro-3-[[(1R)-1-[2-(1H-indol-6-yl)-3,6-dimethyl-4-oxo-chromen-8-yl]ethyl]amino]-N-methylsulfonyl-pyridine-2-carboxamide

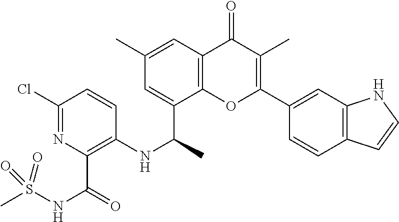 [SMILES: Cc1cc([C@@H](C)Nc2ccc(Cl)nc2C(=O)NS(C)(=O)=O)c2oc(-c3ccc4cc[nH]c4c3)c(C)c(=O)c2c1]

A vial was charged with 6-chloro-3-[[(1R)-1-(2-ethylsulfanyl-3,6-dimethyl-4-oxo-chromen-8-yl)ethyl]amino]-N-methylsulfonyl-pyridine-2-carboxamide (170 mg, 0.33 mmol), 1H-indol-6-ylboronic acid (80.5 mg, 0.50 mmol), copper(I) 3-methylsalicylate (107 mg, 0.50 mmol), tetrakis(triphenylphophine)palladium(0) (77 mg, 0.07 mmol), and EtOH (5 mL). The reaction was degassed with argon, sealed, and stirred at 65° C. for 16 h. After cooling, the reaction was filtered, concentrated under reduced pressure, and purified by silica gel chromatography eluted with 0% to 100% EtOAc in heptanes followed by reversed phase chromatography on C18 eluted with 10% to 100% ACN in 10 mM aqueous NH₄HCO₃ (with 5% MeOH) to give the title compound (28 mg, 15%). ES/MS m/z 565 (M+H).

The following compound in Table 98 was made in a similar way as described for 6-chloro-3-[[(1R)-1-[2-(1H-indol-6-yl)-3,6-dimethyl-4-oxo-chromen-8-yl]ethyl]amino]-N-methylsulfonyl-pyridine-2-carboxamide. Various methods were used to purify the compound, which would be apparent to one skilled in the art.

TABLE 98

| | | | MS ES+ |
|---|---|---|---|
| Example # | Chemical Name | Structure | m/z |
| 215C | 6-Chloro-3-[[(1R)-1- [2-(6-fluoro-2-pyridyl)- 3,6-dimethyl-4-oxo- chromen-8- yl]ethyl]amino]pyridine- 2-carboxamide | 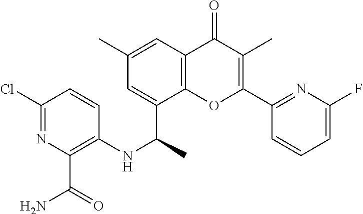 [SMILES: Cc1cc([C@@H](C)Nc2ccc(Cl)nc2C(N)=O)c2oc(-c3cccc(F)n3)c(C)c(=O)c2c1] | 467 [M + H]⁺ |

### Example 216C: 8-[(1R)-1-[[2-(2,6-Difluorophenyl)-3-pyridyl]amino]ethyl]-3,6-dimethyl-2-(3-pyridyl)chromen-4-one

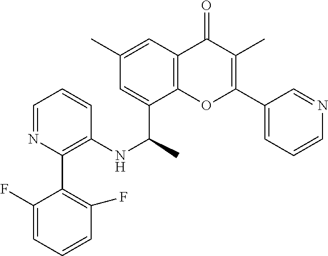 [SMILES: Cc1cc([C@@H](C)Nc2cccnc2-c2c(F)cccc2F)c2oc(-c3cccnc3)c(C)c(=O)c2c1]

Chloro-(2,6-difluorophenyl)zinc was stirred in THF (2 mL) and treated with NMP (1 mL). The reaction was sparged with argon for 5 min and then 8-[(1R)-1-[(2-chloro-3-pyridyl)amino]ethyl]-3,6-dimethyl-2-(3-pyridyl)chromen-4-one (100 mg, 0.25 mmol) and bis(tri-tert-butylphosphine)palladium(O) (12.6 mg, 0.025 mmol) were added and the reaction stirred at 100° C. overnight. Purified by silica gel chromatography eluted with 0% to 100% EtOAc in heptane to give an impure product. This product was purified by reversed phase chromatography eluted with 80% ACN (95:5 ACN in water) in 10 mM aqueous NH₄HCO₃ with 5% MeOH to give the title compound (12.7 mg, 10%). ES/MS m/z 484 (M+H).

### Example 75B: 8-[(1R)-1-[(6-Bromo-2-methyl-3-pyridyl)amino]ethyl]-3,6-dimethyl-2-phenyl-chromen-4-one

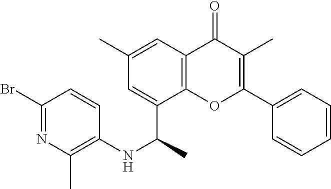 [SMILES: Cc1cc([C@@H](C)Nc2ccc(Br)nc2C)c2oc(-c3ccccc3)c(C)c(=O)c2c1]

A 20 mL vial was charged with 8-[(1S)-1-chloroethyl]-3,6-dimethyl-2-phenyl-chromen-4-one (0.50 g, 1.6 mmol), 6-bromo-2-methyl-pyridin-3-amine (0.45 g, 2.4 mmol), potassium carbonate (0.44 g, 3.2 mmol), and ACN (10 mL). The vial was capped and the reaction stirred at 100° C. for 16 h. After cooling to rt, the reaction was filtered, concentrated, and the residue purified by silica gel chromatography eluted with 0 to 100% EtOAc in heptane. The residue was re-purified by reversed phase C-18 flash chromatography eluted with 0 to 100% ACN in aqueous 10 mM NH₄HCO₃ containing 5% MeOH to give the title compound (0.25 g, 34%). MS ES+ m/z 463/465 [M+H]⁺.

The following compounds in Table 99 were made in a similar way as described for 8-[(1R)-1-[(6-bromo-2-methyl-3-pyridyl)amino]ethyl]-3,6-dimethyl-2-phenyl-chromen-4-one. Various methods were used to purify the compounds, which would be apparent to one skilled in the art.

TABLE 99

| | | | MS ES+ |
|---|---|---|---|
| Example # | Chemical Name | Structure | m/z |
| 217C¹ | 8-[1-[(2-Bromo-6- chloro-3- pyridyl)amino]ethyl]- 3,6-dimethyl-2-(3- pyridyl)chromen-4-one, Isomer 1 | 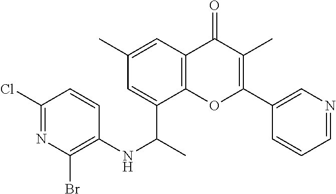 [SMILES: Cc1cc(C(C)Nc2ccc(Cl)nc2Br)c2oc(-c3cccnc3)c(C)c(=O)c2c1] | 484 [M + H]⁺ |
| 218C¹ | 8-[1-[(2-Bromo-6- chloro-3- pyridyl)amino]ethyl]- 3,6-dimethyl-2-(3- pyridyl)chromen-4-one, Isomer 2 | 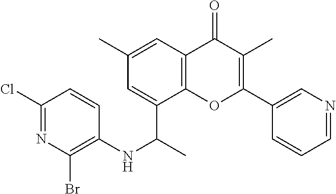 [SMILES: Cc1cc(C(C)Nc2ccc(Cl)nc2Br)c2oc(-c3cccnc3)c(C)c(=O)c2c1] | 484 [M + H]⁺ |

¹There was a loss of chiral purity during the reaction. Chiral separation used the following conditions:
Phenomenex LUX i-Cellulose-5, 30 × 150 mm, 5 μm; 10% to 100% EtOAc in heptane.

### Example 219C and 220C: 8-[1-[[6-Chloro-2-(2-fluorophenyl)-3-pyridyl]amino]ethyl]-3,6-dimethyl-2-(3-pyridyl)chromen-4-one, Isomer 1 and Isomer 2

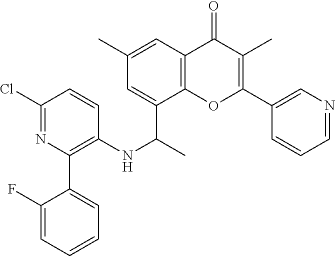 [SMILES: Cc1cc(C(C)Nc2ccc(Cl)nc2-c2ccccc2F)c2oc(-c3cccnc3)c(C)c(=O)c2c1]

Combined 8-[1-[(2-bromo-6-chloro-3-pyridyl)amino]ethyl]-3,6-dimethyl-2-(3-pyridyl)chromen-4-one (60 mg, mmol), (2-fluorophenyl)boronic acid (52 mg, 0.37 mmol), copper(I) thiophene-2-carboxylate (47 mg, 0.25 mmol), and tetrakis(triphenylphosphine)palladium(0) (21 mg, 0.02 mmol) in EtOH (5 mL). The reaction was degassed with argon for 5 min and then stirred at 60° C. for 1 h. The reaction was filtered, the filtrate concentrated under reduced pressure, and the residue purified by silica gel chromatography eluted with 0% to 10000 EtOAc in heptane to give a slightly impure product which was re-purified by reversed phase chromatography eluted with 000 to 100% ACN in 10 mM aqueous NH₄HCO₃ containing 500 MeH. The racemic mixture was separated by chiral SFC [Phenomenex Lux i-Amylose-1, 30×150 mm, 5 μm; 16% MeOH in CO₂]. ES/MS m/z 500 (M+H).

The following compounds in Table 100 were made in a similar way as described for 8-[1-[[6-chloro-2-(2-fluorophenyl)-3-pyridyl]amino]ethyl]-3,6-dimethyl-2-(3-pyridyl)chromen-4-one, Isomer 1 and Isomer 2. Various methods were used to purify the compounds, which would be apparent to one skilled in the art.

TABLE 100

| | | | MS ES+ |
|---|---|---|---|
| Example # | Chemical Name | Structure | m/z |
| 221C¹ | 5-[6-Chloro-3-[1-[3,6- dimethyl-4-oxo-2-(3- pyridyl)chromen-8- yl]ethylamino]-2- pyridyl]-1H-pyridin-2- one, Isomer 1 [SUD- 00001-376] | 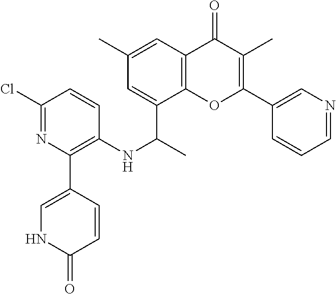 [SMILES: Cc1cc(C(C)Nc2ccc(Cl)nc2-c2ccc(=O)[nH]c2)c2oc(-c3cccnc3)c(C)c(=O)c2c1] | 499 [M + H]⁺ |
| 222C¹ | 5-[6-Chloro-3-[1-[3,6- dimethyl-4-oxo-2-(3- pyridyl)chromen-8- yl]ethylamino]-2- pyridyl]-1H-pyridin-2- one, Isomer 2 | 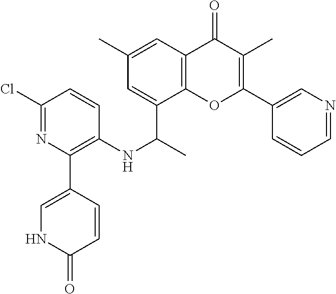 [SMILES: Cc1cc(C(C)Nc2ccc(Cl)nc2-c2ccc(=O)[nH]c2)c2oc(-c3cccnc3)c(C)c(=O)c2c1] | 499 [M + H]⁺ |

¹[Phenomenex Lux i-Amylose-3, 30 × 150 mm, 5 μm; 25% iPrOH (with 0.1% isopropylamine in CO₂]

### Example 76B3: 3,6-Dimethyl-8-[(1R)-1-[(3-methylisothiazol-4-yl)amino]ethyl]-2-(3-pyridyl)chromen-4-one

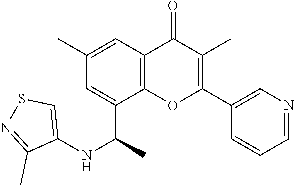 [SMILES: Cc1cc([C@@H](C)Nc2csnc2C)c2oc(-c3cccnc3)c(C)c(=O)c2c1]

Combined 8-[(1R)-1-aminoethyl]-3,6-dimethyl-2-(3-pyridyl)chromen-4-one (200 mg, 0.68 mmol), 4-bromo-3-methylisothiazole (101 mg, 0.57 mmol), potassium phosphate (240 mg, 1.13 mmol), copper(I) iodide (10.8 mg, 0.06 mmol), and ethylene glycol (351 mg, 5.66 mmol) in IPA (1 mL). The reaction was flushed with argon for several minutes before stirring at 80° C. overnight. The reaction was recharged with reactants and continued stirring at 80° C. The reaction was filtered through diatomaceous earth and washed with DCM. The filtrate was concentrated and purified by silica gel chromatography eluted with 0 to 100% EtOAc in heptane to provide an impure compound which was purified by reversed phase chromatography eluted with 80% ACN (with 0.1% formic acid) in water (with 0.1% formic acid) to give the title compound (3.8 mg, 2%). MS ES+ m/z 392 [M+H]⁺.

The following compound in Table 101 was made in a similar way as described for 3,6-dimethyl-8-[(1R)-1-[(3-methylisothiazol-4-yl)amino]ethyl]-2-(3-pyridyl)chromen-4-one. Various methods were used to purify the compound, which would be apparent to one skilled in the art.

TABLE 101

| | | | MS ES+ |
|---|---|---|---|
| Example # | Chemical Name | Structure | m/z |
| 77B¹ | 3,6-Dimethyl-8-[(1R)- 1-[(3-methylisothiazol- 5-yl)amino]ethyl]-2-(3- pyridyl)chromen-4-one | 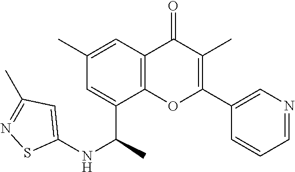 [SMILES: Cc1cc([C@@H](C)Nc2cc(C)ns2)c2oc(-c3cccnc3)c(C)c(=O)c2c1] | 392 [M + H]⁺ |

### Example 223C: 8-[(1R)-1-[2-(Hydroxymethyl)anilino]ethyl]-3,6-dimethyl-2-phenyl-chromen-4-one

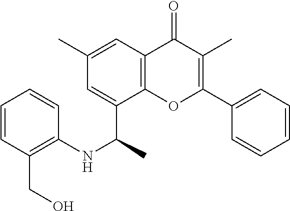 [SMILES: Cc1cc([C@@H](C)Nc2ccccc2CO)c2oc(-c3ccccc3)c(C)c(=O)c2c1]

A solution of 2-[[(1R)-1-(3,6-dimethyl-4-oxo-2-phenyl-chromen-8-yl)ethyl]amino]benzaldehyde (100 mg, 0.25 mmol) in DCM (1 mL) and MeOH (1 mL) was cooled to 0° C. and treated with sodium borohydride (9.52 mg, 0.25 mmol). The reaction was stirred at 0° C. for 30 min. The reaction was diluted with saturated aqueous ammonium chloride and extracted with DCM and EtOAc. The organic layers were combined, dried over Na₂SO₄, filtered, and concentrated under reduced pressure. The residue was purified by silica gel chromatography eluted with 0% to 55% EtOAc in hexane to give the title compound (62.2 mg, 62%). ES-MS m/z 400 (M+H).

The following compound in Table 102 was made in a similar way as described for 8-[(1R)-1-[2-(hydroxymethyl)anilino]ethyl]-3,6-dimethyl-2-phenyl-chromen-4-one. Various methods were used to purify the compound, which would be apparent to one skilled in the art.

TABLE 102

| | | | MS ES+ |
|---|---|---|---|
| Example # | Chemical Name | Structure | m/z |
| 224C | 8-[(1R)-1-[2- (Hydroxymethyl)anilino] ethyl]-3,6-dimethyl- 2-(3-pyridyl)chromen- 4-one | 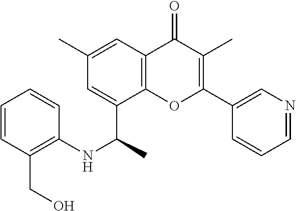 [SMILES: Cc1cc([C@@H](C)Nc2ccccc2CO)c2oc(-c3cccnc3)c(C)c(=O)c2c1] | 401 [M + H]⁺ |

### Example 225C and Example 226C: 3,6-Dimethyl-2-phenyl-8-[(1R)-1-[2-(2,2,2-trifluoro-1-hydroxy-ethyl)anilino]ethyl]chromen-4-one, Isomer 1 and Isomer 2

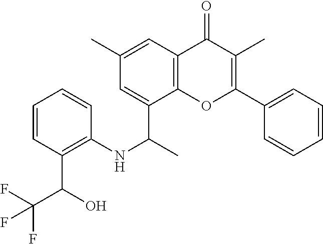 [SMILES: Cc1cc(C(C)Nc2ccccc2C(O)C(F)(F)F)c2oc(-c3ccccc3)c(C)c(=O)c2c1]

2-[[(1R)-1-(3,6-Dimethyl-4-oxo-2-phenyl-chromen-8-yl)ethyl]amino]benzaldehyde (271 mg, 0.68 mmol) and trifluoromethyltrimethylsilane (116 mg, 0.82 mmol) were combined in DMF (2 mL) and treated with potassium carbonate (0.94 mg, 0.007 mmol). The reaction was stirred at rt for 5 min. The reaction was quenched with HCl and the reaction stirred until the silyl ether intermediate consumed. The reaction was neutralized with NaOH, extracted with DCM (3×), dried over Na₂SO₄, filtered, and concentrated under reduced pressure. The residue was purified by reversed phase chromatography on C18 eluted with 20% to 100% ACN in 10 mM aqueous NH₄HCO₃ (with 5% MeOH) to give the racemic compound (14.2 mg, 4%). ES/MS m/z 468 (M+H). The racemic mixture was separated by chiral SFC [Chiralpak IG, 30×250 mm; 10% EtOH in CO₂] to give the title compounds Isomer 1 (first-eluting; 2.67 mg, >99% ee, 0.8%) and Isomer 2 (6.15 mg, 95.6% ee, 2%). MS-ES m/z 468 (M+H).

The following compounds in Table 103 were made in a similar way as described for 3,6-dimethyl-2-phenyl-8-[(1R)-1-[2-(2,2,2-trifluoro-1-hydroxy-ethyl)anilino]ethyl]chromen-4-one, Isomer 1 and Isomer 2. Various methods were used to purify the compounds, which would be apparent to one skilled in the art.

TABLE 103

| | | | MS ES+ |
|---|---|---|---|
| Example # | Chemical Name | Structure | m/z |
| 227C¹ | 3,6-Dimethyl-2-(3- pyridyl)-8-[(1R)-1-[2- (2,2,2-trifluoro-1- hydroxy- ethyl)anilino]ethyl] chromen-4-one, Isomer 1 | 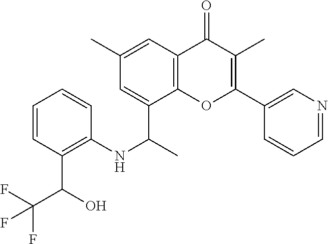 [SMILES: Cc1cc(C(C)Nc2ccccc2C(O)C(F)(F)F)c2oc(-c3cccnc3)c(C)c(=O)c2c1] | 469 [M + H]⁺ |
| 228C¹ | 3,6-Dimethyl-2-(3- pyridyl)-8-[(1R)-1-[2- (2,2,2-trifluoro-1- hydroxy- ethyl)anilino]ethyl] chromen-4-one | 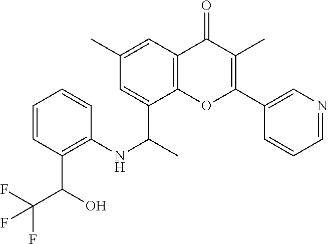 [SMILES: Cc1cc(C(C)Nc2ccccc2C(O)C(F)(F)F)c2oc(-c3cccnc3)c(C)c(=O)c2c1] | 469 [M + H]⁺ |

¹[Chiralcel OD-H, 20 × 150 mm; 15% MeOH in CO₂]

### Example 229C and Example 230C: 8-[(1R)-1-[(6-Chloro-2-methylsulfinyl-3-pyridyl)amino]ethyl]-3,6-dimethyl-2-phenyl-chromen-4-one, Isomer 1 and Isomer 2

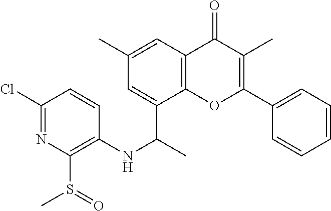 [SMILES: Cc1cc(C(C)Nc2ccc(Cl)nc2S(C)=O)c2oc(-c3ccccc3)c(C)c(=O)c2c1]

A solution of 8-[(1R)-1-[(6-chloro-2-methylsulfanyl-3-pyridyl)amino]ethyl]-3,6-dimethyl-2-phenyl-chromen-4-one (150 mg, 0.33 mmol) in DCM (3 mL) was treated dropwise with mCPBA (51.66 mg, 0.30 mmol) at rt. The reaction was stirred at rt for 4 h and then concentrated under reduced pressure. The residue was purified by silica gel chromatography eluted with 25% EtOAc in petroleum ether to give the title compound (54 mg, 35%) as a white solid. ES-MS m/z 467 (M+H). The racemic mixture was separated by chiral HPLC [Chiral Art Cellulose-SZ, 2.0×25 cm, 5 μm; 10% EtOH in hexane (10 mM NH₃ in MeOH)] to give the title compounds Isomer 1 (first-eluting; 9.4 mg, 17%) and Isomer 2 (18.3 mg, 34%). ES/MS m/z 467 (M+H).

### Example 27B: 8-[(1R)-1-[(6-Chloro-3-pyridyl)amino]ethyl]-2-(1H-indazol-6-yl)-3,6-dimethyl-chromen-4-one

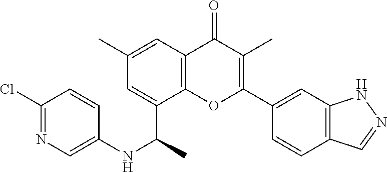 [SMILES: Cc1cc([C@@H](C)Nc2ccc(Cl)nc2)c2oc(-c3ccc4cn[nH]c4c3)c(C)c(=O)c2c1]

To a solution of 8-[(1R)-1-[(6-chloro-3-pyridyl)amino]ethyl]-3,6-dimethyl-2-(1-tetrahydropyran-2-ylindazol-6-yl)chromen-4-one (0.070 g, 0.13 mmol) in DCM (2 mL) was added TFA (0.051 mL, 0.66 mmol). The reaction was stirred at rt for 5 h. Added TFA (0.18 mL, 2.3 mmol). The reaction was stirred at rt overnight and purified by reverse phase chromatography eluted with a gradient of 20 to 100% ACN in aqueous NH₄HCO₃ (10 mM, plus 5% MeOH) to give the title compound (0.038 g, 65%). MS ES+ m/z 445 [M+H]⁺.

The following compound in Table 104 was made in a similar way as described for 8-[(1R)-1-[(6-chloro-2-methoxy-3-pyridyl)amino]ethyl]-3,6-dimethyl-2-(3-pyridyl)chromen-4-one. Various methods were used to purify the compound, which would be apparent to one skilled in the art.

TABLE 104

| | | | MS ES+ |
|---|---|---|---|
| Example # | Chemical Name | Structure | m/z |
| 78B | 8-[(1R)-1-[(6-Chloro- 2-methyl-3- pyridyl)amino]ethyl]-2- (1H-indazol-6-yl)-3,6- dimethyl-chromen-4- one | 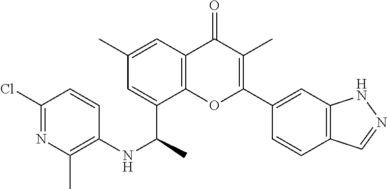 | 445 [M + H]⁺ |
| 231C | 6-Chloro-3-[[(1R)-1- [2-(1H-indol-2-yl)-3,6- dimethyl-4-oxo- chromen-8- yl]ethyl]amino]-N- methylsulfonyl- pyridine-2- carboxamide | 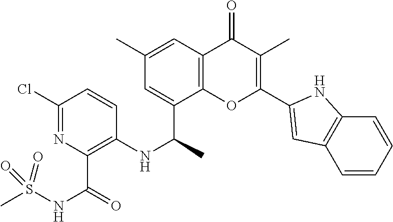 [SMILES: Cc1cc([C@@H](C)Nc2ccc(Cl)nc2C(=O)NS(C)(=O)=O)c2oc(-c3cc4ccccc4[nH]3)c(C)c(=O)c2c1] | 565 [M + H]⁺ |

### Example 79B: 2-[4-(Azetidin-3-yl)phenyl]-8-[(1R)-1-[(6-chloro-3-pyridyl)amino]ethyl]-3,6-dimethyl-chromen-4-one

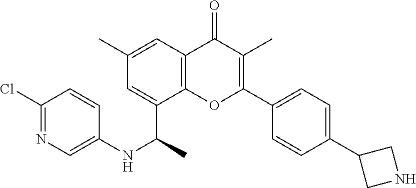 [SMILES: Cc1cc([C@@H](C)Nc2ccc(Cl)nc2)c2oc(-c3ccc(C4CNC4)cc3)c(C)c(=O)c2c1]

A solution of tert-butyl 3-[4-[8-[(1R)-1-[(6-chloro-3-pyridyl)amino]ethyl]-3,6-dimethyl-4-oxo-chromen-2-yl]phenyl]azetidine-1-carboxylate (0.05 g, 0.09 mmol) in DCM was treated with HCl in 1,4-dioxane (4M, 0.05 g, 1.3 mmol) and allowed to stir until the reaction was complete. The reaction was concentrated and purified by reversed phase C18 flash chromatography eluted with 10 to 72% ACN in 10 mM aqueous NH₄HCO₃ with 5% MeOH to give the title compound (30 mg, 69%).

### Example 232C: 8-[(1R)-1-[(2-Hydroxy-3-pyridyl)amino]ethyl]-3,6-dimethyl-2-(3-pyridyl)chromen-4-one

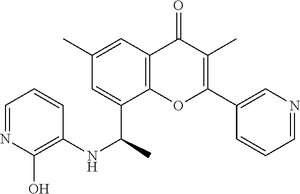 [SMILES: Cc1cc([C@@H](C)Nc2cccnc2O)c2oc(-c3cccnc3)c(C)c(=O)c2c1]

Combined 8-[(1R)-1-[(2-benzyloxy-3-pyridyl)amino]ethyl]-3,6-dimethyl-2-(3-pyridyl)chromen-4-one (35 mg, 0.07 mmol) with trifluoroacetic acid (1.1 mL) and stirred at 50° C. for 16 h. After cooling to rt, the reaction was concentrated under reduced pressure and the residue purified by reversed phase chromatography eluted with a gradient of 10% to 90% ACN in water (with 0.1% TFA). The fractions containing product were pooled, concentrated under reduced pressure, and filtered using bicarbonate resin to give the title compound (16.5 mg, 58%). ES/MS m/z 388 (M+H).

### Example 233C: 8-[(1R)-1-[(6-Chloro-2-piperazin-1-yl-3-pyridyl)amino]ethyl]-3,6-dimethyl-2-(3-pyridyl)chromen-4-one

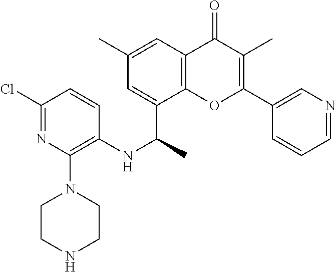 [SMILES: Cc1cc([C@@H](C)Nc2ccc(Cl)nc2N2CCNCC2)c2oc(-c3cccnc3)c(C)c(=O)c2c1]

A vial containing a mixture of tert-butyl 4-[6-chloro-3-[[(1R)-1-[3,6-dimethyl-4-oxo-2-(3-pyridyl)chromen-8-yl]ethyl]amino]-2-pyridyl]piperazine-1-carboxylate (0.29 g, 0.49 mmol) and 1,4-dioxane (3 mL) was treated with 4M HCl in 1,4-dioxane (1.2 mL) and stirred at rt for 2 h and 50° C. for 2 h. The reaction was cooled to rt, concentrated under reduced pressure, and the residue purified by reversed phase chromatography on C18 eluted with 1000 to 100% ACN (with 0.10% TFA) in water (with 0.1% TFA) to give the title compound (0.17 g, 710%) as a white solid. ES/MS m/z 490 (M).

The following compounds in Table 105 were made in a similar way as described for 8-[(1R)-1-[(6-Chloro-2-piperazin-1-yl-3-pyridyl)amino]ethyl]-3,6-dimethyl-2-(3-pyridyl)chromen-4-one. Various methods were used to purify the compounds, which would be apparent to one skilled in the art.

TABLE 105

| | | | MS ES+ |
|---|---|---|---|
| Example # | Chemical Name | Structure | m/z |
| 234C | 8-[(1R)-1-[[6-Chloro- 2-(4-hydroxy-1- piperidyl)-3- pyridyl]amino]ethyl]- 3,6-dimethyl-2-(3- pyridyl)chromen-4-one | 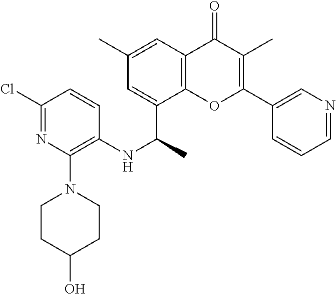 [SMILES: Cc1cc([C@@H](C)Nc2ccc(Cl)nc2N2CCC(O)CC2)c2oc(-c3cccnc3)c(C)c(=O)c2c1] | 505 [M + H]⁺ |
| 235C | 8-[(1R)-1-[[6-Chloro- 2-[(3S,4S)-3,4- dihydroxypyrrolidin-1- yl]-3- pyridyl]amino]ethyl]- 3,6-dimethyl-2-(3- pyridyl)chromen-4-one | 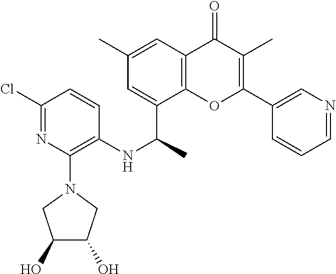 [SMILES: Cc1cc([C@@H](C)Nc2ccc(Cl)nc2N2C[C@H](O)[C@@H](O)C2)c2oc(-c3cccnc3)c(C)c(=O)c2c1] | 507 [M + H]⁺ |
| 236C | 3-[6-Chloro-3-[[(1R)- 1-[2-[1-(2,3- dihydroxypropyl)pyrazol- 4-yl]-3,6-dimethyl- 4-oxo-chromen-8- yl]ethyl]amino]-2- pyridyl]-4H-1,2,4- oxadiazol-5-one | 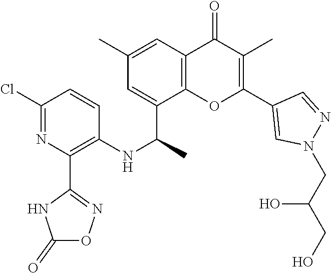 [SMILES: Cc1cc([C@@H](C)Nc2ccc(Cl)nc2-c2noc(=O)[nH]2)c2oc(-c3cnn(CC(O)CO)c3)c(C)c(=O)c2c1] | 553 [M + H]⁺ |

### Example 28B and Example 29B: 8-[(1R)-1-[2-(1,2-Dihydroxyethyl)anilino]ethyl]-3,6-dimethyl-2-phenyl-chromen-4-one and 8-[(1R)-1-[2-(2-Hydroxyacetyl)anilino]ethyl]-3,6-dimethyl-2-phenyl-chromen-4-one

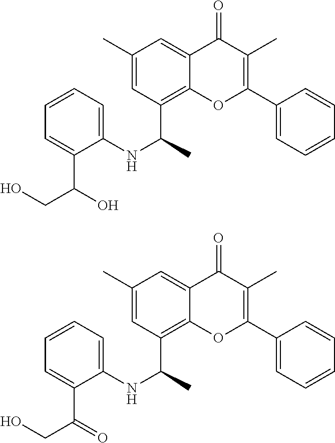 [SMILES: Cc1cc([C@@H](C)Nc2ccccc2C(O)CO)c2oc(-c3ccccc3)c(C)c(=O)c2c1 ; Cc1cc([C@@H](C)Nc2ccccc2C(=O)CO)c2oc(-c3ccccc3)c(C)c(=O)c2c1]

To a solution of 3,6-dimethyl-2-phenyl-8-[(1R)-1-(2-vinylanilino)ethyl]chromen-4-one (0.082 g, 0.21 mmol) in acetone (1 mL) and water (0.25 mL) was added N-methylmorpholine N-oxide (0.073 g, 0.62 mmol) and osmium tetroxide (0.0016 mL, 0.031 mmol). The reaction was stirred at 25° C. for 2 h and purified by reverse phase chromatography eluted with a gradient of 10 to 100% ACN in aqueous NH₄HCO₃ (10 mM, plus 5% MeOH) to give the diol (0.035 g, 39%, ˜2:3 mix of diastereomers) and the hydroxy ketone (0.0060 g, 7%). MS ES+ m/z 430 [M+H]⁺ and 428 [M+H]⁺, respectively.

### Example 80B: 8-[(1R)-1-[(6-Chloro-2-methylsulfonyl-3-pyridyl)amino]ethyl]-3,6-dimethyl-2-phenyl-chromen-4-one

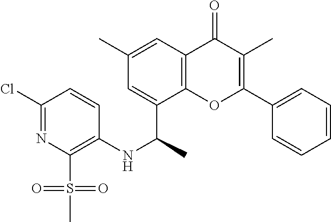 [SMILES: Cc1cc([C@@H](C)Nc2ccc(Cl)nc2S(C)(=O)=O)c2oc(-c3ccccc3)c(C)c(=O)c2c1]

A solution of 8-[(1R)-1-[(6-chloro-2-methylsulfanyl-3-pyridyl)amino]ethyl]-3,6-dimethyl-2-phenyl-chromen-4-one (0.15 g, 0.33 mmol) in DCM (2 mL) was treated with mCPBA (0.17 g, 1.00 mmol) dropwise at rt under nitrogen. After addition, the reaction was allowed to stir for 4 hr at rt. The reaction was concentrated and purified by silica gel chromatography eluted with EtOAc in petroleum ether (1:3). The resulting material was re-purified by preparative HPLC (Xselect CSH C18 OBD column) eluted with 58 to 66% ACN in water (with 0.1% formic acid) to give the title compound (71.9 mg, 45%) as a white solid. MS ES+ m/z 483 [M+H]⁺.

PI3K-Alpha Kinase (PIK3CA) Activity, H1047R Mutant In Vitro Cell Based Assay and Determining IC50 Values for Inhibitors

The MDA-MB-453 (ATCC-HTB-131) cell line was obtained from the American Type Culture Collection (Manassas, VA). Cells were maintained in Dulbecco's Modified Eagle Media (DMEM, Gibco 11965-092) supplemented with 10% Fetal Bovine Serum, heat inactivated (FBS HI, Gibco 10082-147), 1× non-essential amino acids (NEAA, Gibco 11140-050), and 1 mM sodium pyruvate (Gibco 11360-070). Cultures were maintained in a humidified incubator at 37° C. under 5% CO₂/95% air.

For compound testing in 0% FBS, MDA-MB-453 cells were seeded at a density of 1.5×10⁴ cells per well in white 384-well plates in 20 μl of Minimum Essential Media (MEM) assay media with 1×NEAA, 1 mM sodium pyruvate, and 1 μg/mL human insulin (Sigma I9278). Compounds dissolved in 10 mM stock solutions in DMSO were serially diluted 1:3 in DMSO to generate a 10-point dilution series and plated using an acoustic liquid handler system (Echo 550 Series Liquid Handler, Labcyte). A 5× intermediate compound dilution plate in MEM with 1×NEAA and 1 mM sodium pyruvate (150 μM starting compound concentration in 1.5% DMSO) was then prepared. Five μl of the intermediate serially diluted compounds were added to the cell plate to final concentrations ranging from 30 mM to 0.0015 mM in 0.3% DMSO. 0.3% DMSO alone was used to establish the maximum (MAX) signal and GDC-0032 at a final concentration of 1 μM was used as a reference compound for the minimum (MIN) signal. After 3 hours treatment, the medium was removed, and the cells lysed in 10 μL of 1× SureFire Lysis buffer with shaking for 10 minutes at room temperature. The Acceptor Mix (Reaction Buffer 1+Reaction Buffer 2+Activation Buffer+SureFire Ultra Acceptor Beads) was prepared by diluting Activation buffer 25-fold in combined Reaction Buffer 1 and Reaction Buffer 2. The Acceptor beads were diluted 50-fold in the combined Reaction Buffers. Five μL of Acceptor Mix was added to each well, the plate was sealed and covered with foil and incubated for 1 hour at room temperature. The Donor Mix (dilution buffer+SureFire Ultra Donor Beads) was prepared by diluting Donor Beads 50-fold in dilution buffer. Five μL of the Donor Mix was added to each well and the plate sealed and covered with foil and incubated for 1 hour at room temperature in the dark. The plates were read on a Neo2 plate reader instrument from Biotek using standard AlphaLisa settings. Compounds were tested in duplicate and the average % inhibition at each compound concentration was used to generate a single dose response curve. The data were processed using the Genedata-Screener tool. Relative IC₅₀ values were determined using luminescence units by calculating percent inhibition with respect to the in-plate “MIN” (GDC-0032 reference control) and “MAX” (DMSO) controls. The data was analyzed using a 4-parameter nonlinear logistic equation (four-parameter logistic concentration-response curve):

Y=bottom+[(top−bottom)/1+(X/IC50)slope]

where Y=% inhibition, X=concentration of inhibitor, bottom=minimum value of y attained by curve-fit, top=maximum value of y attained by curve-fit and slope=steepness of curve at the IC₅₀.

% Inhibition=[(signal at X−median Min)/(median Max−median Min)]×100

IC₅₀: concentration of compound that reduces a given response (ligand binding, enzyme response) by 50%. Relative IC₅₀: concentration giving half the compound's maximum response.

In the above cell based assay the compounds of Examples 1A-3A, 5A-22A, 24A, 27A, 30A-32A, 34A-36A, 39A-46A, 48A-63A, 65A, 66A, 68A-81A, 83A, 84A, 86A-94A, 96A-181A, 183A-265A, 4B-7B, 9B, 10B, 12B, 14B, 16B-38B, 42-80B, 1C-54C, 56C-76C, 78C-80C, 82C-127C, 129C-141C, 143C-201C, 203C-217C, 219C, 222C, 224C-244C were tested and all exhibited IC₅₀ values of less than 1 μM for the PI3K-Alpha kinase H1047R mutant.

In the above cell based assay the compounds of Examples 1A-3A, 7A-22A, 24A, 32A, 34A-36A, 39A-45A, 49A-51A, 53A-61A, 63A, 66A, 68A, 72A, 74A, 76A-81A, 83A, 84A, 86A, 89A-92A, 96A-175A, 177A, 178A, 187A-198A, 200A-206A, 208A-236A, 241A-255A, 258A-264A, 4B-7B, 9B, 10B, 14B, 17B-19B, 22B, 24B-38B, 42B-58B, 61-70B, 72-76B, 78B, and 80B 4C-40C, 42C-51C, 53C, 54C, 56C-65C, 67C-76C, 78C-80C, 82C-97C, 99C-104C, 106C-126C, 129C, 130C, 132C, 133C, 135C-141C, 143C-166C, 168C, 170C-200C, 203C-205C, 207C-209C, 212C-217C, 219C, 222C, 224C, 226C-231C, and 233C-244C were tested and all exhibited IC₅₀ values of less than 500 nM for the PI3K-Alpha kinase H1047R mutant.

1. A compound of a formula: 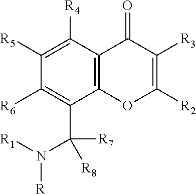
or a pharmaceutically acceptable salt thereof, wherein:
R and R₁ together with the nitrogen to which they are attached form an optionally substituted bicyclic ring selected from dihydrobenzimidazolone, dihydroindazolone, indolinone or quinazolinone; wherein the optionally substituted bicyclic ring is optionally substituted with one to three substituents each independently selected from oxo, —CN, halogen, C₁-C₆ alkyl, C₁-C₆ haloalkyl, —OH or C₁-C₆ alkoxy; or
R is —H or C₁-C₃ alkyl; and
R₁ is an optionally substituted bicyclic ring selected from isobenzofuranone, benzofuranone, indole, isoindolinone, indolinone, quinazolinone, 3,4-dihydro-2H-isoquinolin-1-one, 2H-isoquinolin-1-one, imidaza[1,2-A]pyridine, or benzothiazolone; wherein the optionally substituted bicyclic ring is optionally substituted with one to three substituents each independently selected from oxo, —CN, halogen, C₁-C₆ alkyl, C₁-C₆ haloalkyl, —OH or C₁-C₆ alkoxy; or
R₁ is a group of the formula: 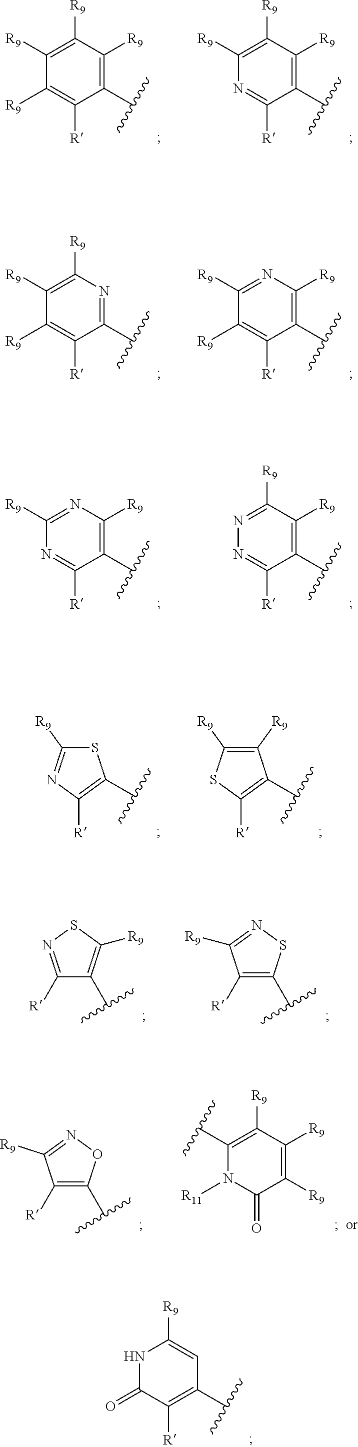
R′ is hydrogen, halogen, C₁-C₃ alkyl, C₁-C₃ haloalkyl, C₁-C₃ hydroxyalkyl, C₁-C₃ alkoxy, C₁-C₃ haloalkoxy, —OH, —CH(OH)—CH₂OH, —CH(OH)C₁-C₃ haloalkyl, —C(O)—CH₂OH, C₃-C₆ cycloalkyl, —NO₂, —NR₁₁R₁₁, —N(R₁₁)—CO₂C₁-C₃ alkyl, —N(R₁₁)—SO₂C₁-C₃ alkyl, —N(R₁₁)—SO₂R₁₅, —SO₂C₁-C₃ alkyl, —S(O)C₁-C₃ alkyl, —SO₂NR₁₁R₁₁, —SO₂N(R₁₁)—C(O)—C₁-C₃ alkyl, —SO₂N(R₁₁)—CN, —SO₂N(R₁₁)(R₁₃)—C(═N—OH)—NH₂, —CN, —CO₂C₁-C₃ alkyl, —CONR₁₁R₁₂, —C(O)N(R₁₁)—(CH₂)ₙ—R₁₃, —C(O)—SR₁₂, —C(O)—NHSO₂R₁₆, —C(O)CH═SOR₁₁(R₁₁), or —C(O)CH₂CN, or a group of the formula: 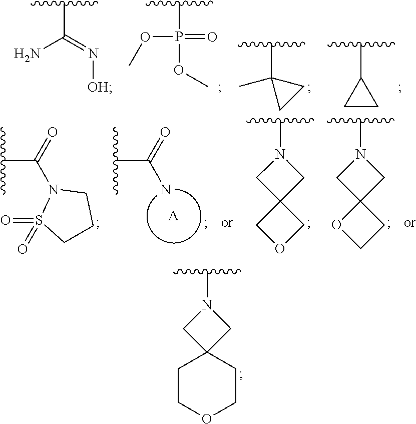
wherein ring A is pyrrolidine optionally substituted with a —CN; or
R′ is selected from oxetane, azetidine, pyrrolidine, tetrahydrofuran, morpholine, thiomorpholine, piperidine, piperazine, pyrrole, furan, thiophene, pyrazole, imidazole, isoxazole, oxazole, isothiazole, thiazole, triazole, oxadiazole, 1,2,4-oxadiazolin-5-one, thiadiazole, tetrazole, phenyl, pyridine, pyridazine, pyrimidine, pyrazine, or triazine; each of which is optionally substituted with one to three substituents independently selected from oxo, —OH, —NR₁₁R₁₁, —N(R₁₁C(O)—R₁₁, —N(R₁₁)—CN, —OR₁₁, —CN, halogen, morpholine, oxetane, C₁-C₆ haloalkyl or C₁-C₆ alkyl optionally substituted with an aryl, a 5-member heteroaryl or a 6-member heteroaryl; or
R′ is a group of the formula: 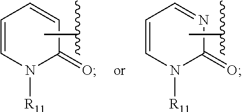
or
R₂ is a group of the formula: 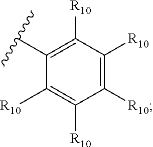
R₂ is a group of the formula: 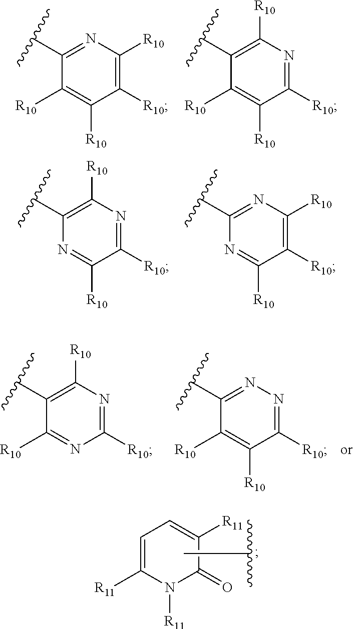
or
R₂ is an optionally substituted 5-member ring heteroaryl selected from pyrrole, furan, thiophene, pyrazole, isoxazole, isothiazole, imidazole, oxazole, thiazole, triazole, tetrazole, oxadiazole, and thiadiazole; wherein the optionally substituted 5-member ring heteroaryl is optionally substituted with one to three substituents each independently selected from —CN, halogen, C₁-C₆ haloalkyl, C₁-C₆ alkoxy, C₁-C₆ haloalkoxy, —SO₂R₁₁, —CO₂C₁-C₃ alkyl, —C(O)NR₁₁R₁₁, —OH, —NR₁₁R₁₁, —NR₁₁CO₂R₁₁, an optionally substituted C₁-C₆ alkyl, an optionally substituted C₂-C₆ alkenyl, an optionally substituted C₂-C₆ alkynyl, an optionally substituted C₃-C₅ cycloalkyl, an optionally substituted heterocycle selected from pyrrolidine, pyrrolidinone, piperidine, tetrahydropyran, oxetane, azetidine, or morpholine, an optionally substituted phenyl, an optionally substituted 1,3-benzodioxole, an optionally substituted 2,3-dihydro-1,4-benzodioxine, or an optionally substituted heteroaryl selected from pyridine, pyrimidine, pyridazine, pyrazine, pyrazole, isoxazole, isothiazole, imidazole, oxazole, or thiazole; wherein the optionally substituted C₁-C₆ alkyl, C₂-C₆ alkenyl, or C₂-C₆ alkynyl is each optionally substituted with a —CN, —OH, oxetanyl, C₁-C₃ alkoxy, —CH₂CH(OH)CH(OH)R₁₁, or —CONR₁₁R₁₁;
the optionally substituted C₃-C₅ cycloalkyl, phenyl, 1,3-benzodioxole, 2,3-dihydro-1,4-benzodioxine, heterocycle or heteroaryl is each optionally substituted with one to three substituents each independently selected from halogen, C₁-C₃ alkyl, C₁-C₃ haloalkyl, C₁-C₃ alkoxy, C₁-C₃ haloalkoxy, —C(O)(CH₂)ₙOR₁₁, C(O)C(CH₃)₂OH, —SO₂R₁₁, —NR₁₁R₁₁, —OH or —CN; or
R₂ is an optionally substituted bicyclic ring selected from 1,3-benzodioxole, 2,3-dihydro-1,4-benzodioxine, indole, indazole, isoindazole, isoindolin-1-one, indolin-2-one, benzoxazole, benzotriazole, benzo[d]oxazol-2(3H)-one, 1,3-dihydro-2H-pyrrolo[2,3-b]pyridin-2-one, 6,7-dihydro-4H-pyrazolo[5,1-c][1,4]oxazine, 4,5,6,7-tetrahydropyrazolo[1,5-a]pyrazine, or 2,3-dihydro-[1,4]dioxino[2,3-b]pyridine, imidazo[1,2-a]pyridine, pyrazolo[4,3-b]pyridine, pyrazolo[3,4-b]pyridine, pyrazolo[3,4-c]pyridine, pyrazolo[1,5-a]pyrimidine, or an optionally substituted bicyclic heteroaryl of 8 to 10 ring atoms containing 1, 2, 3, 4, or 5 ring heteroatoms independently selected from N, O, or S; wherein the optionally substituted bicyclic ring is optionally substituted with one to three substituents each independently selected from halogen and C₁-C₆ alkyl; the optionally substituted bicyclic heteroaryl is optionally substituted with one to three substituents each independently selected from —CN, halogen, C₁-C₆ haloalkyl, C₁-C₆ alkoxy, C₁-C₆ haloalkoxy, —SO₂R₁₁, —CO₂CH, —CO₂C₁-C₃ alkyl, —CONR₁₁R₁₁, —NR₁₁R₁₁, —NR₁₁CO₂R₁₁, —OH, an optionally substituted C₁-C₆ alkyl, an optionally substituted C₂-C₆ alkenyl, an optionally substituted C₂-C₆ alkynyl, an optionally substituted C₃-C₅ cycloalkyl, an optionally substituted heterocycle selected from pyrrolidine, pyrrolidinone, piperidine or morpholine, an optionally substituted phenyl, an optionally substituted 1,3-benzodioxole, an optionally substituted 2,3-dihydro-1,4-benzodioxine, or an optionally substituted heteroaryl selected from pyridine, pyrimidine, pyridazine, pyrazine, pyrazole, isoxazole, isothiazole, imidazole, oxazole, or thiazole; wherein the optionally substituted C₁-C₆ alkyl, C₂-C₆ alkenyl, or C₂-C₆ alkynyl is each optionally substituted with a —CN, —OH, oxetanyl, C₁-C₃ alkoxy, —C(O)NR₁₁R₁₁ or phenyl; the optionally substituted C₃-C₅ cycloalkyl, phenyl, 1,3-benzodioxole, 2,3-dihydro-1,4-benzodioxine, heterocycle or heteroaryl is each optionally substituted with one to three substituents each independently selected from halogen, C₁-C₃ alkyl, C₁-C₃ haloalkyl, C₁-C₃ alkoxy, C₁-C₃ haloalkoxy, —SO₂R₁₁, —NR₁₁R₁₁, —OH or —CN; or
R₂ is a cyclopropyl, cyclobutyl, cyclopentyl, bicyclo[1.1.1]pentane, bicyclo[2.2.2]octane, or cyclohexyl, each optionally substituted with one to three R₁₀ substituents, or R₂ is a group of the formula: 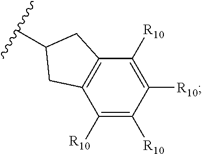
and
R₃ is —H, halogen, —CN, —C(CN)═CHOH, —N(H)(C₁-C₃ alkyl), —N(C₁-C₃ alkyl)₂, —N(H)(CH₂CH₂CO₂H), —CO—C₁-C₃ alkyl, C₁-C₆ alkyl, C₁-C₆ haloalkyl, C₁-C₆ hydroxyalkyl, C₃-C₅ cycloalkyl, an optionally substituted heterocycle of 3 to 5 ring atoms containing 1, 2, or 3 ring heteroatoms independently selected from N, O, or S, or an optionally substituted heteroaryl of 5 or 6 ring atoms containing 1, 2, or 3 ring heteroatoms independently selected from N, O, or S; wherein the optionally substituted heterocycle or heteroaryl is each optionally substituted with one to three substituents each independently selected from halogen, C₁-C₃ alkyl, or C₁-C₃ haloalkyl;
each of R₄, R₅ and R₆ is independently —H, halogen, —CN, C₁-C₆ alkyl or C₁-C₆ haloalkyl;
R₇ is —CN, C₁-C₆ alkyl, —CH₂OH or C₁-C₆ haloalkyl;
R₈ is —H or C₁-C₆ alkyl;
each R₉ is independently —H, halogen, —CN, C₁-C₆ alkyl, C₁-C₆ haloalkyl, C₁-C₆ alkoxy, or C₃-C₅ cycloalkyl;
each R₁₀ is independently —H, —CN, halogen, C₁-C₆ haloalkyl, C₁-C₆ alkoxy, C₁-C₆ haloalkoxy, —SO₂R₁₁, —SONR₁₁R₁₁, —CO₂H, —CO₂C₁-C₃ alkyl, —C(O)NR₁₁R₁₂, —NR₁₁R₁₁, —NR₁₁—CO₂R₁₁, —N(R₁₁)COR₁₁, —OH, an optionally substituted C₁-C₆ alkyl, an optionally substituted C₂-C₆ alkenyl, an optionally substituted C₂-C₆ alkynyl, an optionally substituted C₃-C₅ cycloalkyl, an optionally substituted heterocycle selected from pyrrolidine, pyrrolidinone, piperidine or morpholine, an optionally substituted phenyl, an optionally substituted 1,3-benzodioxole, an optionally substituted 2,3-dihydro-1,4-benzodioxine, or an optionally substituted heteroaryl selected from pyrazole, isoxazole, isothiazole, imidazole, oxazole, thiazole, or pyridine, or a group of the formula: 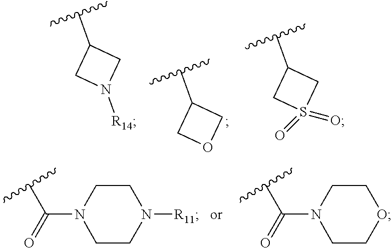
wherein the optionally substituted C₁-C₆ alkyl, C₂-C₆ alkenyl, or C₂-C₆ alkynyl is each optionally substituted with a —CN, —OH, oxetanyl, C₁-C₃ alkoxy, or —CONR₁₁R₁₁; the optionally substituted C₃-C₅ cycloalkyl, phenyl, 1,3-benzodioxole, 2,3-dihydro-1,4-benzodioxine, heterocycle or heteroaryl is each optionally substituted with one to three substituents each independently selected from halogen, C₁-C₃ alkyl, C₁-C₃ haloalkyl, C₁-C₃ alkoxy, C₁-C₃ haloalkoxy, —SO₂R₁₁, —NR₁₁R₁₁, —OH or —CN;
each R₁₁ is independently —H, C₁-C₃ alkyl, C₃-C₇ cycloalkyl, or C₁-C₃ haloalkyl;
each R₁₂ is independently —H, optionally substituted C₁-C₃ alkyl, C₃-C₆ alkoxy, C₃-C₆ cycloalkyl, —SO₂C₁-C₃ alkyl, —SO₂C₁-C₃ haloalkyl, —SO₂NR₁₁R₁₁, —NR₁₁R₁₁, —OR₁₁, —O—CH₂—CH(OH)—CH₂OH, —CN, oxetane, tetrahydrofuran, an aryl, a 5-member heteroaryl optionally substituted with a methyl, a 6-member heteroaryl, or a group of the formula 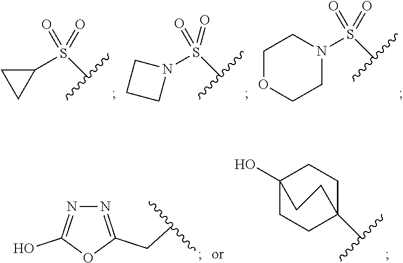
wherein the optionally substituted C₁-C₃ alkyl is optionally substituted with —OH, a C₃-C₆ cycloalkyl, oxetane, tetrahydrofuran, an aryl, a 5-member heteroaryl, a 6-member heteroaryl or an indole;
R₁₃ is —NR₁₁R₁₁, —OR₁₁, —SO₂C₁-C₃ alkyl, or a ring selected from oxetane, tetrahydrofuran or oxadiazole wherein the ring is optionally substituted with —NR₁₁R₁₁, or —OR₁₁;
R₁₄ is —H, optionally substituted C₁-C₃ alkyl, —SO₂C₁-C₃ alkyl, an aryl, a 5-member heteroaryl, or a 6-member heteroaryl; wherein the optionally substituted C₁-C₃ alkyl is optionally substituted with an aryl, a 5-member heteroaryl or a 6-member heteroaryl;
R₁₅ is an optionally substituted aryl, or an optionally substituted 6-member heteroaryl;
wherein the optionally substituted aryl, or the optionally substituted 6-member heteroaryl is each optionally substituted with one to three substituents each independently selected from halogen, C₁-C₃ alkyl, or C₁-C₃ haloalkyl;
R₁₆ is H, C₁-C₃ alkyl, —NH₂, phenyl, or pyridine, and
n is 0, 1 or 2.

2. The compound of claim 1, or pharmaceutically acceptable salt thereof, wherein:
R and R₁ together with the nitrogen to which they are attached form an optionally substituted bicyclic ring selected from dihydrobenzimidazolone, dihydroindazolone, indolinone or quinazolinone; wherein the optionally substituted bicyclic ring is optionally substituted with one to three substituents each independently selected from oxo, —CN, halogen, C₁-C₆ alkyl, C₁-C₆ haloalkyl, —OH or C₁-C₆ alkoxy; or
R is —H or C₁-C₃ alkyl;
R₁ is an optionally substituted bicyclic ring selected from isobenzofuranone, benzofuranone, isoindolinone, indolinone, quinazolinone, 3,4-dihydro-2H-isoquinolin-1-one, 2H-isoquinolin-1-one, or benzothiazolone; wherein the optionally substituted bicyclic ring is optionally substituted with one to three substituents each independently selected from oxo, —CN, halogen, C₁-C₆ alkyl, C₁-C₆ haloalkyl, —OH or C₁-C₆ alkoxy; or
R₁ is a group of the formula: 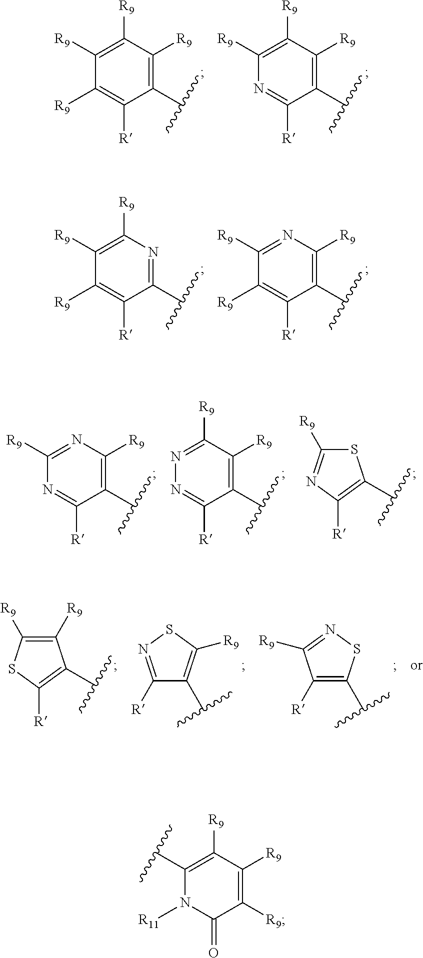
R′ is hydrogen, halogen, C₁-C₃ alkyl, C₁-C₃ haloalkyl, C₁-C₃ alkoxy, C₁-C₃ haloalkoxy, —OH, —CH(OH)—CH₂OH, —CO—CH₂OH, C₃-C₆ cycloalkyl, —NO₂, —N(R₁₁)—CO₂C₁-C₃ alkyl, —N(R₁₁)—SO₂C₁-C₃ alkyl, —N(R₁₁)—SO₂R₁₅, —SO₂C₁-C₃ alkyl, —SO₂NR₁₁R₁₁, —SO₂N(R₁₁)—CO—C₁-C₃ alkyl, —SO₂N(R₁₁)—CN, —C(═N—OH)—NH₁₂, —CN, —CO₂C₁-C₃ alkyl, —CONR₁₁R₁₂, —CON(R₁₁)(CH₂)ₙ—R₁₃, —CO—SR₁₂, or a group of the formula: 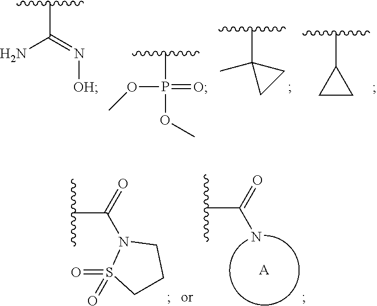
wherein ring A is pyrrolidine optionally substituted with a —CN; or
R′ is selected from oxetane, pyrrolidine, tetrahydrofuran, morpholine, piperidine, piperazine, pyrrole, furan, thiophene, pyrazole, imidazole, isoxazole, oxazole, isothiazole, thiazole, triazole, oxadiazole, thiadiazole, tetrazole, phenyl, pyridine, pyridazine, pyrimidine, pyrazine, or triazine; each of which is optionally substituted with one to three substituents independently selected from oxo, —OH, —NR₁₁R₁₁, —N(R₁₁)—CO—R₁₁, —N(R₁₁)—CN, —OR₁₁, —CN, halogen, C₁-C₆ haloalkyl or C₁-C₆ alkyl optionally substituted with an aryl, a 5-member heteroaryl or a 6-member heteroaryl; or
R′ is a group of the formula: 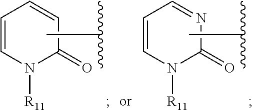
R₂ is a group of the formula: 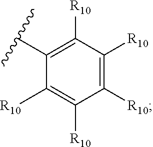
or
R₂ is a group of the formula: 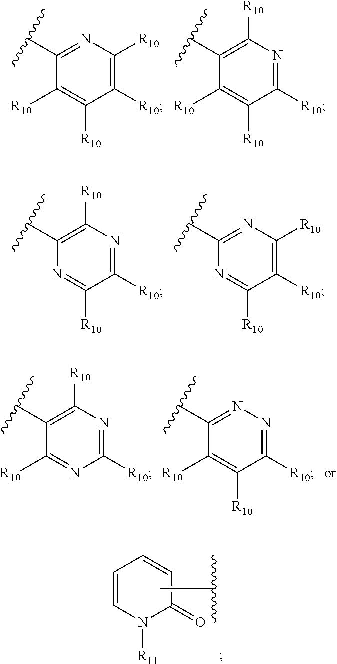
or
R₂ is an optionally substituted 5-member ring heteroaryl selected from pyrrole, furan, thiophene, pyrazole, isoxazole, isothiazole, imidazole, oxazole, thiazole, triazole, tetrazole, oxadiazole, and thiadiazole; wherein the optionally substituted 5-member ring heteroaryl is optionally substituted with one to three substituents each independently selected from —CN, halogen, C₁-C₆ haloalkyl, C₁-C₆ alkoxy, C₁-C₆ haloalkoxy, —SO₂R₁₁, —CO₂C₁-C₃ alkyl, —CONR₁₁R₁₁, —OH, —NR₁₁R₁₁, —NR₁₁CO₂R₁₁, an optionally substituted C₁-C₆ alkyl, an optionally substituted C₂-C₆ alkenyl, an optionally substituted C₂-C₆ alkynyl, an optionally substituted C₃-C₅ cycloalkyl, an optionally substituted heterocycle selected from pyrrolidine, pyrrolidinone, piperidine or morpholine, an optionally substituted phenyl, an optionally substituted 1,3-benzodioxole, an optionally substituted 2,3-dihydro-1,4-benzodioxine, or an optionally substituted heteroaryl selected from pyridine, pyrimidine, pyridazine, pyrazine, pyrazole, isoxazole, isothiazole, imidazole, oxazole, or thiazole; wherein the optionally substituted C₁-C₆ alkyl, C₂-C₆ alkenyl, or C₂-C₆ alkynyl is each optionally substituted with a —CN, —OH, oxetanyl, C₁-C₃ alkoxy, or —CONR₁₁R₁₁; the optionally substituted C₃-C₅ cycloalkyl, phenyl, 1,3-benzodioxole, 2,3-dihydro-1,4-benzodioxine, heterocycle or heteroaryl is each optionally substituted with one to three substituents each independently selected from halogen, C₁-C₃ alkyl, C₁-C₃ haloalkyl, C₁-C₃ alkoxy, C₁-C₃ haloalkoxy, —SO₂R₁₁, —NR₁₁R₁₁, —OH or —CN; or
R₂ is an optionally substituted bicyclic ring selected from 1,3-benzodioxole, 2,3-dihydro-1,4-benzodioxine, isoindolin-1-one, indolin-2-one, benzo[d]oxazol-2(3H)-one, 1,3-dihydro-2H-pyrrolo[2,3-b]pyridin-2-one, or 2,3-dihydro-[1,4]dioxino[2,3-b]pyridine, or an optionally substituted bicyclic heteroaryl of 8 to 10 ring atoms containing 1, 2, 3, 4, or 5 ring heteroatoms independently selected from N, O, or S; wherein the optionally substituted bicyclic ring is optionally substituted with one to three substituents each independently selected from halogen and C₁-C₆ alkyl; the optionally substituted bicyclic heteroaryl is optionally substituted with one to three substituents each independently selected from —CN, halogen, C₁-C₆ haloalkyl, C₁-C₆ alkoxy, C₁-C₆ haloalkoxy, —SO₂R₁₁, —CO₂C₁-C₃ alkyl, —CONR₁₁R₁₁, —NR₁₁R₁₁, —NR₁₁CO₂R₁₁, —OH, an optionally substituted C₁-C₆ alkyl, an optionally substituted C₂-C₆ alkenyl, an optionally substituted C₂-C₆ alkynyl, an optionally substituted C₃-C₅ cycloalkyl, an optionally substituted heterocycle selected from pyrrolidine, pyrrolidinone, piperidine or morpholine, an optionally substituted phenyl, an optionally substituted 1,3-benzodioxole, an optionally substituted 2,3-dihydro-1,4-benzodioxine, or an optionally substituted heteroaryl selected from pyridine, pyrimidine, pyridazine, pyrazine, pyrazole, isoxazole, isothiazole, imidazole, oxazole, or thiazole; wherein the optionally substituted C₁-C₆ alkyl, C₂-C₆ alkenyl, or C₂-C₆ alkynyl is each optionally substituted with a —CN, —OH, oxetanyl, C₁-C₃ alkoxy, —CONR₁₁R₁₁ or phenyl; the optionally substituted C₃-C₅ cycloalkyl, phenyl, 1,3-benzodioxole, 2,3-dihydro-1,4-benzodioxine, heterocycle or heteroaryl is each optionally substituted with one to three substituents each independently selected from halogen, C₁-C₃ alkyl, C₁-C₃ haloalkyl, C₁-C₃ alkoxy, C₁-C₃ haloalkoxy, —SO₂R₁₁, —NR₁₁R₁₁, —OH or —CN; or
R₂ is a cyclopropyl, cyclobutyl, cyclopentyl, or cyclohexyl, each optionally substituted with one to three R₁₀ substituents, or R₂ is a group of the formula: 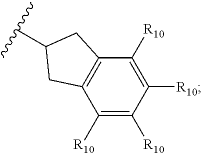
R₃ is —H, halogen, —CN, —N(H)(C₁-C₃ alkyl), —N(C₁-C₃ alkyl)₂, —N(H)(CH₂CH₂CO₂H), —CO—C₁-C₃ alkyl, C₁-C₆ alkyl, C₁-C₆ haloalkyl, C₁-C₆ hydroxyalkyl, C₃-C₅ cycloalkyl, an optionally substituted heterocycle of 3 to 5 ring atoms containing 1, 2, or 3 ring heteroatoms independently selected from N, O, or S, or an optionally substituted heteroaryl of 5 or 6 ring atoms containing 1, 2, or 3 ring heteroatoms independently selected from N, O, or S; wherein the optionally substituted heterocycle or heteroaryl is each optionally substituted with one to three substituents each independently selected from halogen, C₁-C₃ alkyl, or C₁-C₃ haloalkyl;
each of R₄, R₅ and R₆ is independently —H, halogen, C₁-C₆ alkyl or C₁-C₆ haloalkyl;
R₇ is —CN, C₁-C₆ alkyl or C₁-C₆ haloalkyl;
R₈ is —H or C₁-C₆ alkyl;
each R₉ is independently —H, halogen, —CN, C₁-C₆ alkyl, C₁-C₆ haloalkyl, C₁-C₆ alkoxy, or C₃-C₅ cycloalkyl;
each R₁₀ is independently —H, —CN, halogen, C₁-C₆ haloalkyl, C₁-C₆ alkoxy, C₁-C₆ haloalkoxy, —SO₂R₁₁, —SONR₁₁R₁₁, —CO₂H, —CO₂C₁-C₃ alkyl, —CONR₁₁R₁₂, —NR₁₁R₁₁, —NR₁₁—CO₂R₁₁, —OH, an optionally substituted C₁-C₆ alkyl, an optionally substituted C₂-C₆ alkenyl, an optionally substituted C₂-C₆ alkynyl, an optionally substituted C₃-C₅ cycloalkyl, an optionally substituted heterocycle selected from pyrrolidine, pyrrolidinone, piperidine or morpholine, an optionally substituted phenyl, an optionally substituted 1,3-benzodioxole, an optionally substituted 2,3-dihydro-1,4-benzodioxine, or an optionally substituted heteroaryl selected from pyrazole, isoxazole, isothiazole, imidazole, oxazole, thiazole, or pyridine, or a group of the formula: 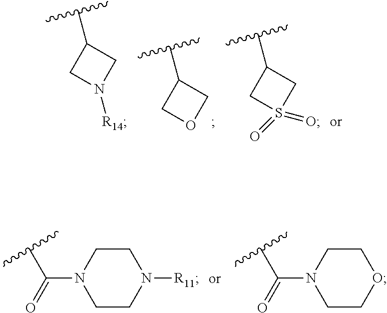
wherein the optionally substituted C₁-C₆ alkyl, C₂-C₆ alkenyl, or C₂-C₆ alkynyl is each optionally substituted with a —CN, —OH, oxetanyl, C₁-C₃ alkoxy, or —CONR₁₁R₁₁; the optionally substituted C₃-C₅ cycloalkyl, phenyl, 1,3-benzodioxole, 2,3-dihydro-1,4-benzodioxine, heterocycle or heteroaryl is each optionally substituted with one to three substituents each independently selected from halogen, C₁-C₃ alkyl, C₁-C₃ haloalkyl, C₁-C₃ alkoxy, C₁-C₃ haloalkoxy, —SO₂R₁₁, —NR₁₁R₁₁, —OH or —CN;
each R₁₁ is independently —H or C₁-C₃ alkyl;
each R₁₂ is independently —H, optionally substituted C₁-C₃ alkyl, C₃-C₆ cycloalkyl; —SO₂C₁-C₃ alkyl, —SO₂C₁-C₃ haloalkyl, —SO₂NR₁₁R₁₁, —NR₁₁R₁₁, —OR₁₁, —O—CH₂—CH(OH)—CH₂OH, —CN, oxetane, tetrahydrofuran, an aryl, a 5-member heteroaryl optionally substituted with a methyl, a 6-member heteroaryl, or a group of the formula 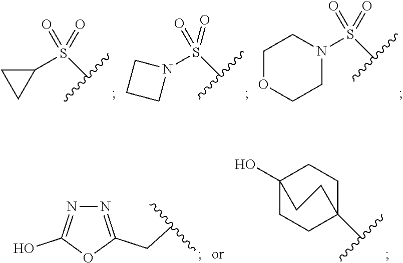
wherein the optionally substituted C₁-C₃ alkyl is optionally substituted with —OH, a C₃-C₆ cycloalkyl, oxetane, tetrahydrofuran, an aryl, a 5-member heteroaryl, a 6-member heteroaryl or an indole;

3. The compound of claim 1, or pharmaceutically acceptable salt thereof, wherein R and R₁ together with the nitrogen to which they are attached form an optionally substituted bicyclic ring selected from dihydrobenzimidazolone, dihydroindazolone, indolinone or quinazolinone; wherein the optionally substituted bicyclic ring is optionally substituted with one to three substituents each independently selected from oxo, —CN, halogen, C₁-C₆ alkyl, C₁-C₆ haloalkyl, —OH or C₁-C₆ alkoxy; or
R is —H or C₁-C₃ alkyl; and
R₁ is an optionally substituted bicyclic ring selected from isobenzofuranone, benzofuranone, isoindolinone, indolinone, quinazolinone, or benzothiazolone; wherein the optionally substituted bicyclic ring is optionally substituted with one to three substituents each independently selected from oxo, —CN, halogen, C₁-C₆ alkyl, C₁-C₆ haloalkyl, —OH or C₁-C₆ alkoxy; or
R₁ is a group of the formula: 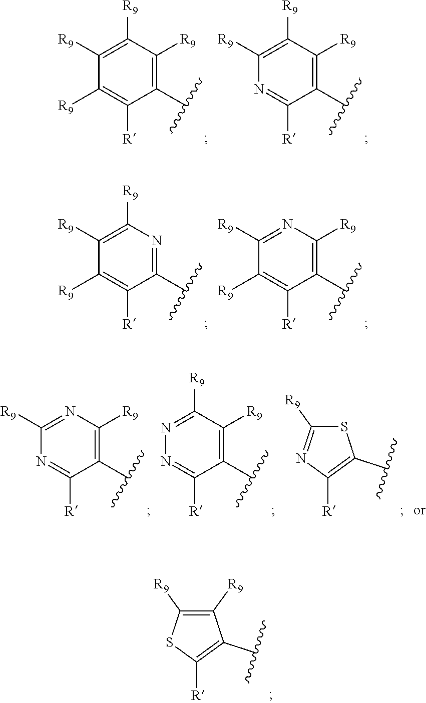
R′ is C₁-C₃ haloalkyl, C₁-C₃ alkoxy, —NO₂, —N(R₁₁)—CO₂C₁-C₃ alkyl, —N(R₁₁)—SO₂C₁-C₃ alkyl, —SO₂NR₁₁R₁₁, —SO₂N(R₁₁)—CO—C₁-C₃ alkyl, —C(═N—OH)—NH₂, —CN, —CO₂C₁-C₃ alkyl, —CONR₁₁R₁₂, —CON(R)—(CH₂)ₙ—R₁₃, or a group of the formula 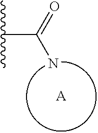
wherein ring A is pyrrolidine optionally substituted with a —CN; or
R′ is selected from oxetane, pyrrolidine, tetrahydrofuran, pyrrole, furan, thiophene, pyrazole, imidazole, isoxazole, oxazole, isothiazole, thiazole, triazole, oxadiazole, thiadiazole, tetrazole, pyridine, pyridazine, pyrimidine, pyrazine, or triazine; each of which is optionally substituted with one to three substituents independently selected from oxo, —OH, —NR₁₁R₁₁, —N(R₁₁)—CO—R₁₁, —N(R₁₁)—CN, —OR₁₁, —CN, C₁-C₆ haloalkyl or C₁-C₆ alkyl optionally substituted with an aryl, a 5-member heteroaryl or a 6-member heteroaryl;
R₂ is a group of the formula: 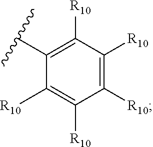
or
R₂ is a group of the formula: 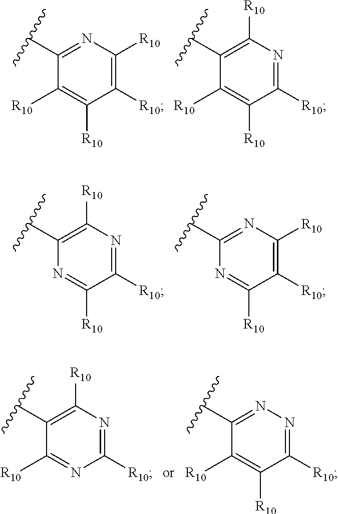
or
R₂ is an optionally substituted 5-member ring heteroaryl selected from pyrrole, furan, thiophene, pyrazole, isoxazole, isothiazole, imidazole, oxazole, thiazole, triazole, tetrazole, oxadiazole, and thiadiazole; wherein the optionally substituted 5-member ring heteroaryl is optionally substituted with one to three substituents each independently selected from —CN, halogen, C₁-C₆ haloalkyl, C₁-C₆ alkoxy, C₁-C₆ haloalkoxy, —SO₂R₁₁, —CO₂C₁-C₃ alkyl, —CONR₁₁R₁₁, —OH, —NR₁₁R₁₁, —NR₁₁CO₂R₁₁, an optionally substituted C₁-C₆ alkyl, an optionally substituted C₂-C₆ alkenyl, an optionally substituted C₂-C₆ alkynyl, an optionally substituted C₃-C₅ cycloalkyl, an optionally substituted heterocycle selected from pyrrolidine, pyrrolidinone, piperidine or morpholine, an optionally substituted phenyl, an optionally substituted 1,3-benzodioxole, an optionally substituted 2,3-dihydro-1,4-benzodioxine, or an optionally substituted heteroaryl selected from pyridine, pyrimidine, pyridazine, pyrazine, pyrazole, isoxazole, isothiazole, imidazole, oxazole, or thiazole; wherein the optionally substituted C₁-C₆ alkyl, C₂-C₆ alkenyl, or C₂-C₆ alkynyl is each optionally substituted with a —CN, —OH, oxetanyl, C₁-C₃ alkoxy, or —CONR₁₁R₁₁; the optionally substituted C₃-C₅ cycloalkyl, phenyl, 1,3-benzodioxole, 2,3-dihydro-1,4-benzodioxine, heterocycle or heteroaryl is each optionally substituted with one to three substituents each independently selected from halogen, C₁-C₃ alkyl, C₁-C₃ haloalkyl, C₁-C₃ alkoxy, C₁-C₃ haloalkoxy, —SO₂R₁₁, —NR₁₁R₁₁, —OH or —CN; or
R₂ is an optionally substituted bicyclic ring selected from 1,3-benzodioxole, 2,3-dihydro-1,4-benzodioxine, isoindolin-1-one, indolin-2-one, benzo[d]oxazol-2(3H)-one, 1,3-dihydro-2H-pyrrolo[2,3-b]pyridin-2-one, or 2,3-dihydro-[1,4]dioxino[2,3-b]pyridine, or an optionally substituted bicyclic heteroaryl of 8 to 10 ring atoms containing 1, 2, 3, 4, or 5 ring heteroatoms independently selected from N, O, or S; wherein the optionally substituted bicyclic ring is optionally substituted with one to three substituents each independently selected from halogen and C₁-C₆ alkyl; the optionally substituted bicyclic heteroaryl is optionally substituted with one to three substituents each independently selected from —CN, halogen, C₁-C₆ haloalkyl, C₁-C₆ alkoxy, C₁-C₆ haloalkoxy, —SO₂R₁₁, —CO₂C₁-C₃ alkyl, —CONR₁₁R₁₁, —NR₁₁R₁₁, —NR₁₁CO₂R₁₁, —OH, an optionally substituted C₁-C₆ alkyl, an optionally substituted C₂-C₆ alkenyl, an optionally substituted C₂-C₆ alkynyl, an optionally substituted C₃-C₅ cycloalkyl, an optionally substituted heterocycle selected from pyrrolidine, pyrrolidinone, piperidine or morpholine, an optionally substituted phenyl, an optionally substituted 1,3-benzodioxole, an optionally substituted 2,3-dihydro-1,4-benzodioxine, or an optionally substituted heteroaryl selected from pyridine, pyrimidine, pyridazine, pyrazine, pyrazole, isoxazole, isothiazole, imidazole, oxazole, or thiazole; wherein the optionally substituted C₁-C₆ alkyl, C₂-C₆ alkenyl, or C₂-C₆ alkynyl is each optionally substituted with a —CN, —OH, oxetanyl, C₁-C₃ alkoxy, —CONR₁₁R₁₁ or phenyl; the optionally substituted C₃-C₅ cycloalkyl, phenyl, 1,3-benzodioxole, 2,3-dihydro-1,4-benzodioxine, heterocycle or heteroaryl is each optionally substituted with one to three substituents each independently selected from halogen, C₁-C₃ alkyl, C₁-C₃ haloalkyl, C₁-C₃ alkoxy, C₁-C₃ haloalkoxy, —SO₂R₁₁, —NR₁₁R₁₁, —OH or —CN; or
R₂ is a cyclopropyl, cyclobutyl, cyclopentyl, or cyclohexyl, each optionally substituted with one to three R₁₀ substituents, or R₂ is a group of the formula: 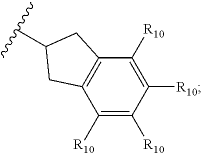
each R₁₀ is independently —H, —CN, halogen, C₁-C₆ haloalkyl, C₁-C₆ alkoxy, C₁-C₆ haloalkoxy, —SO₂R₁₁, —CO₂C₁-C₃ alkyl, —CONR₁₁R₁₁, —NR₁₁R₁₁, —NR₁₁—CO₂R₁₁, —OH, an optionally substituted C₁-C₆ alkyl, an optionally substituted C₂-C₆ alkenyl, an optionally substituted C₂-C₆ alkynyl, an optionally substituted C₃-C₅ cycloalkyl, an optionally substituted heterocycle selected from pyrrolidine, pyrrolidinone, piperidine or morpholine, an optionally substituted phenyl, an optionally substituted 1,3-benzodioxole, an optionally substituted 2,3-dihydro-1,4-benzodioxine, or an optionally substituted heteroaryl selected from pyrazole, isoxazole, isothiazole, imidazole, oxazole, or thiazole; wherein the optionally substituted C₁-C₆ alkyl, C₂-C₆ alkenyl, or C₂-C₆ alkynyl is each optionally substituted with a —CN, —OH, oxetanyl, C₁-C₃ alkoxy, or —CONR₁₁R₁₁; the optionally substituted C₃-C₅ cycloalkyl, phenyl, 1,3-benzodioxole, 2,3-dihydro-1,4-benzodioxine, heterocycle or heteroaryl is each optionally substituted with one to three substituents each independently selected from halogen, C₁-C₃ alkyl, C₁-C₃ haloalkyl, C₁-C₃ alkoxy, C₁-C₃ haloalkoxy, —SO₂R₁₁, —NR₁₁R₁₁, —OH or —CN;
R₁₂ is —H, optionally substituted C₁-C₃ alkyl, —SO₂C₁-C₃ alkyl, —O—CH₂—CH(OH)—CH₂OH, an aryl, a 5-member heteroaryl, or a 6-member heteroaryl; wherein the optionally substituted C₁-C₃ alkyl is optionally substituted with an aryl, a 5-member heteroaryl or a 6-member heteroaryl; and
R₁₃ is —NR₁₁R₁₁, —OR₁₁, oxetane or tetrahydrofuran.

4. The compound of claim 1, or pharmaceutically acceptable salt thereof, wherein R₈ is —H.

5. The compound of claim 1, or pharmaceutically acceptable salt thereof, having the Formula:

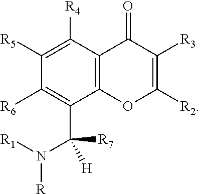

6. The compound of claim 1, or pharmaceutically acceptable salt thereof, wherein R is —H.

7. The compound of claim 1, or pharmaceutically acceptable salt thereof, wherein R₄ is —H or halogen.

8. The compound of claim 1, or pharmaceutically acceptable salt thereof, wherein R₄ is —H.

9. The compound of claim 1, or pharmaceutically acceptable salt thereof having the Formula

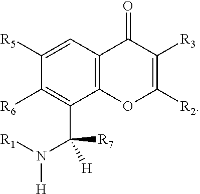

10. The compound of claim 1, or pharmaceutically acceptable salt thereof, wherein R₃ is —H, —CN, C₁-C₃ alkyl, or C₁-C₃ haloalkyl.

11. The compound of claim 1, or pharmaceutically acceptable salt thereof, wherein R₃ is —H, —CN, of claim 1 methyl, or trifluoromethyl.

12. The compound of claim 1, or pharmaceutically acceptable salt thereof, wherein R₃ is —H or methyl.

13. The compound of claim 1, or pharmaceutically acceptable salt thereof, wherein R₅ is —H, halogen, C₁-C₃ alkyl or C₁-C₃ haloalkyl.

14. The compound of claim 1, or pharmaceutically acceptable salt thereof, wherein R₅ is —H, halogen, methyl, or trifluoromethyl.

15. The compound of claim 1, or pharmaceutically acceptable salt thereof, wherein R₆ is —H or halogen.

16. The compound of claim 1 selected from: 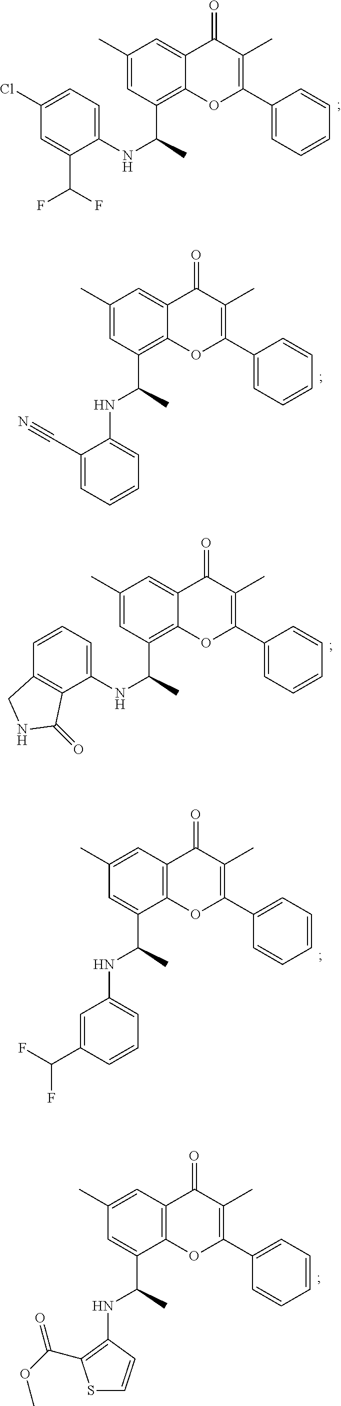 [SMILES: Cc1cc([C@@H](C)Nc2ccc(Cl)cc2C(F)F)c2oc(-c3ccccc3)c(C)c(=O)c2c1 ; Cc1cc([C@@H](C)Nc2ccccc2C#N)c2oc(-c3ccccc3)c(C)c(=O)c2c1 ; Cc1cc([C@@H](C)Nc2cccc3c2C(=O)NC3)c2oc(-c3ccccc3)c(C)c(=O)c2c1 ; Cc1cc([C@@H](C)Nc2cccc(C(F)F)c2)c2oc(-c3ccccc3)c(C)c(=O)c2c1 ; COC(=O)c1sccc1N[C@H](C)c1cc(C)cc2c(=O)c(C)c(-c3ccccc3)oc12] 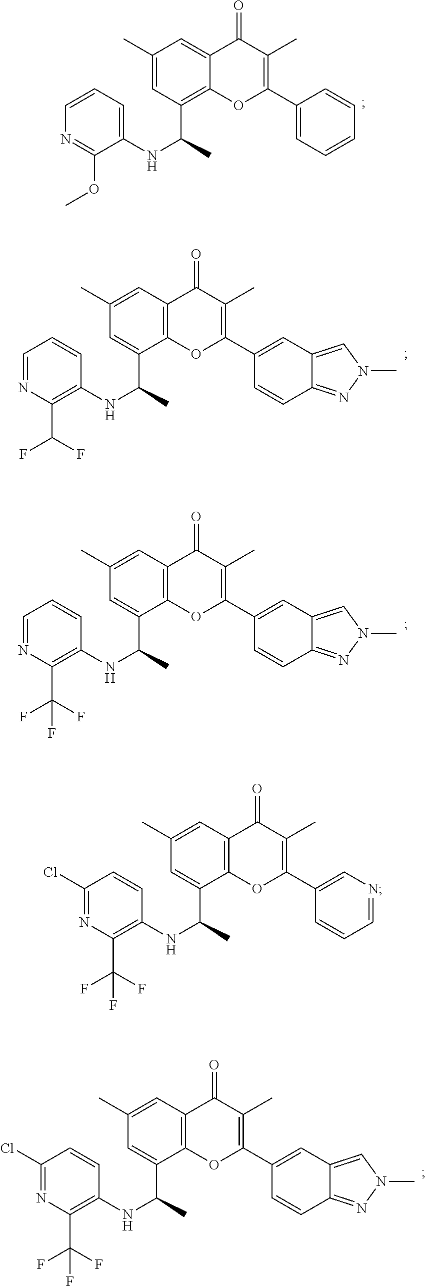 [SMILES: COc1ncccc1N[C@H](C)c1cc(C)cc2c(=O)c(C)c(-c3ccccc3)oc12 ; Cc1cc([C@@H](C)Nc2cccnc2C(F)F)c2oc(-c3ccc4nn(C)cc4c3)c(C)c(=O)c2c1 ; Cc1cc([C@@H](C)Nc2cccnc2C(F)(F)F)c2oc(-c3ccc4nn(C)cc4c3)c(C)c(=O)c2c1 ; Cc1cc([C@@H](C)Nc2ccc(Cl)nc2C(F)(F)F)c2oc(-c3cccnc3)c(C)c(=O)c2c1 ; Cc1cc([C@@H](C)Nc2ccc(Cl)nc2C(F)(F)F)c2oc(-c3ccc4nn(C)cc4c3)c(C)c(=O)c2c1] 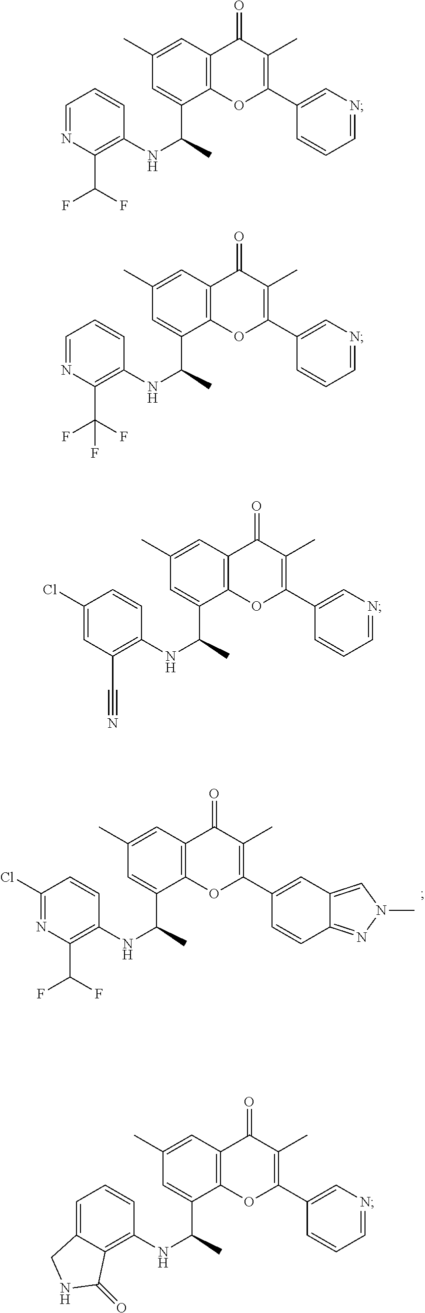 [SMILES: Cc1cc([C@@H](C)Nc2cccnc2C(F)F)c2oc(-c3cccnc3)c(C)c(=O)c2c1 ; Cc1cc([C@@H](C)Nc2cccnc2C(F)(F)F)c2oc(-c3cccnc3)c(C)c(=O)c2c1 ; Cc1cc([C@@H](C)Nc2ccc(Cl)cc2C#N)c2oc(-c3cccnc3)c(C)c(=O)c2c1 ; Cc1cc([C@@H](C)Nc2ccc(Cl)nc2C(F)F)c2oc(-c3ccc4nn(C)cc4c3)c(C)c(=O)c2c1 ; Cc1cc([C@@H](C)Nc2cccc3c2C(=O)NC3)c2oc(-c3cccnc3)c(C)c(=O)c2c1] 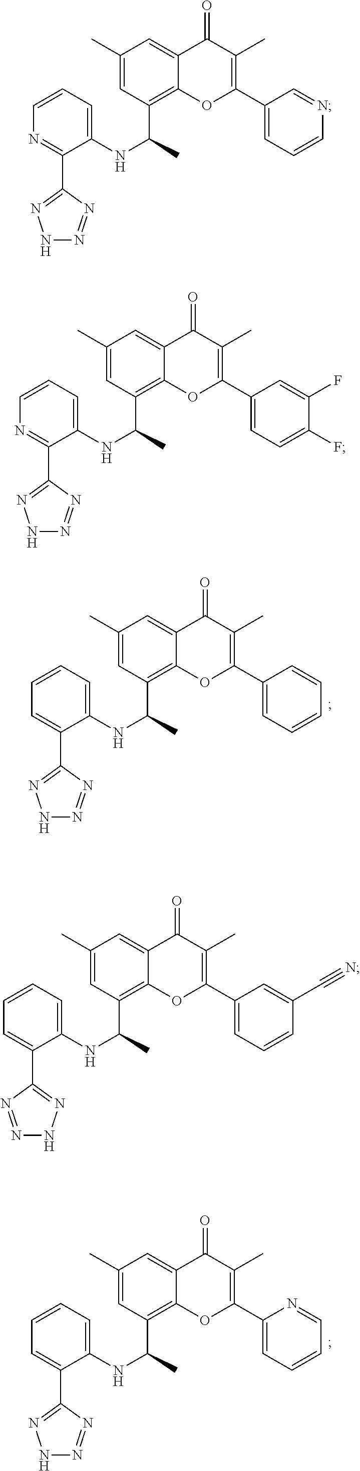 [SMILES: Cc1cc([C@@H](C)Nc2cccnc2-c2nn[nH]n2)c2oc(-c3cccnc3)c(C)c(=O)c2c1 ; Cc1cc([C@@H](C)Nc2cccnc2-c2nn[nH]n2)c2oc(-c3ccc(F)c(F)c3)c(C)c(=O)c2c1 ; Cc1cc([C@@H](C)Nc2ccccc2-c2nn[nH]n2)c2oc(-c3ccccc3)c(C)c(=O)c2c1 ; Cc1cc([C@@H](C)Nc2ccccc2-c2nn[nH]n2)c2oc(-c3cccc(C#N)c3)c(C)c(=O)c2c1 ; Cc1cc([C@@H](C)Nc2ccccc2-c2nn[nH]n2)c2oc(-c3ccccn3)c(C)c(=O)c2c1] 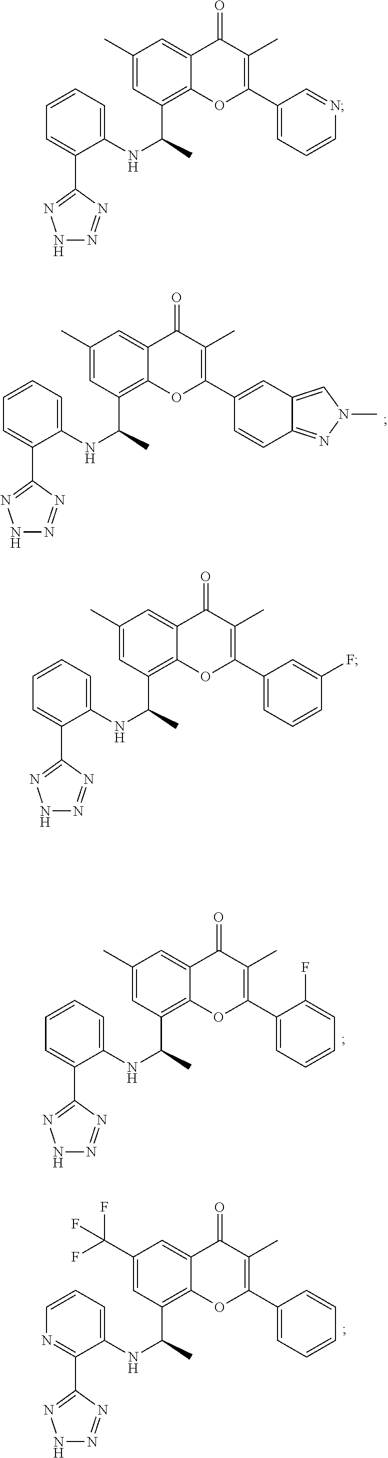 [SMILES: Cc1cc([C@@H](C)Nc2ccccc2-c2nn[nH]n2)c2oc(-c3cccnc3)c(C)c(=O)c2c1 ; Cc1cc([C@@H](C)Nc2ccccc2-c2nn[nH]n2)c2oc(-c3ccc4nn(C)cc4c3)c(C)c(=O)c2c1 ; Cc1cc([C@@H](C)Nc2ccccc2-c2nn[nH]n2)c2oc(-c3cccc(F)c3)c(C)c(=O)c2c1 ; Cc1cc([C@@H](C)Nc2ccccc2-c2nn[nH]n2)c2oc(-c3ccccc3F)c(C)c(=O)c2c1 ; Cc1c(-c2ccccc2)oc2c([C@@H](C)Nc3cccnc3-c3nn[nH]n3)cc(C(F)(F)F)cc2c1=O] 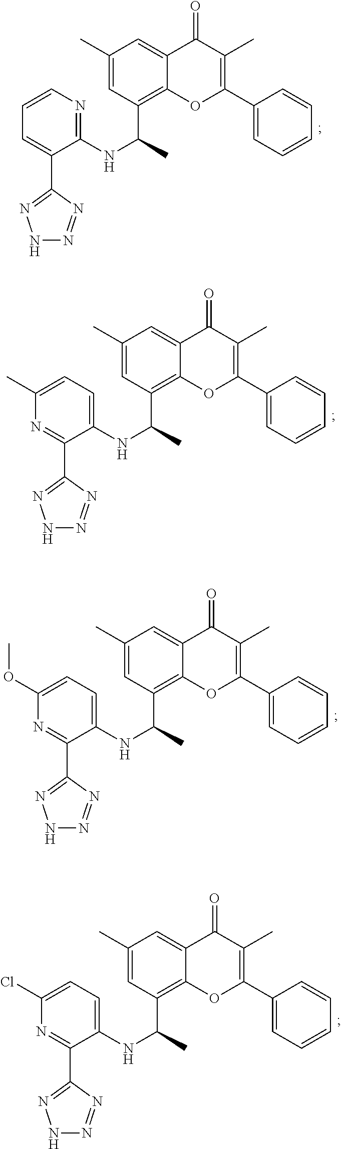 [SMILES: Cc1cc([C@@H](C)Nc2ncccc2-c2nn[nH]n2)c2oc(-c3ccccc3)c(C)c(=O)c2c1 ; Cc1cc([C@@H](C)Nc2ccc(C)nc2-c2nn[nH]n2)c2oc(-c3ccccc3)c(C)c(=O)c2c1 ; COc1ccc(N[C@H](C)c2cc(C)cc3c(=O)c(C)c(-c4ccccc4)oc23)c(-c2nn[nH]n2)n1 ; Cc1cc([C@@H](C)Nc2ccc(Cl)nc2-c2nn[nH]n2)c2oc(-c3ccccc3)c(C)c(=O)c2c1] 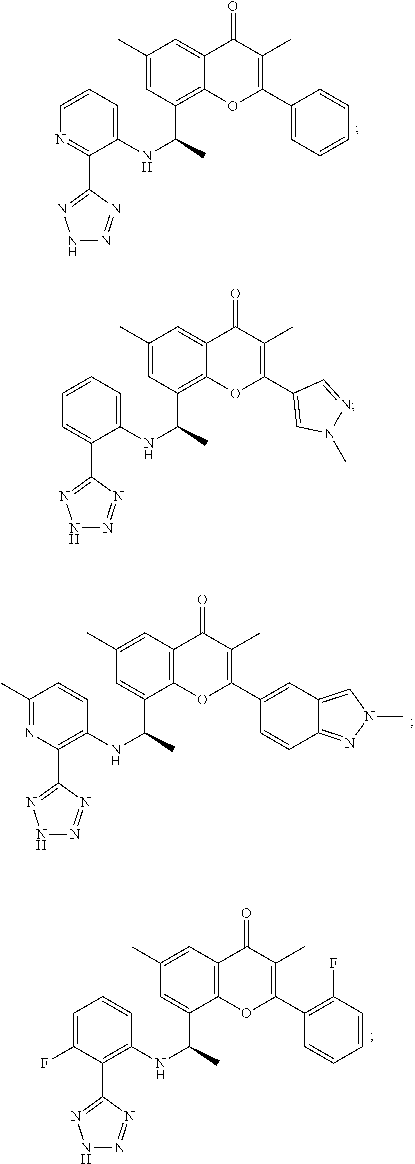 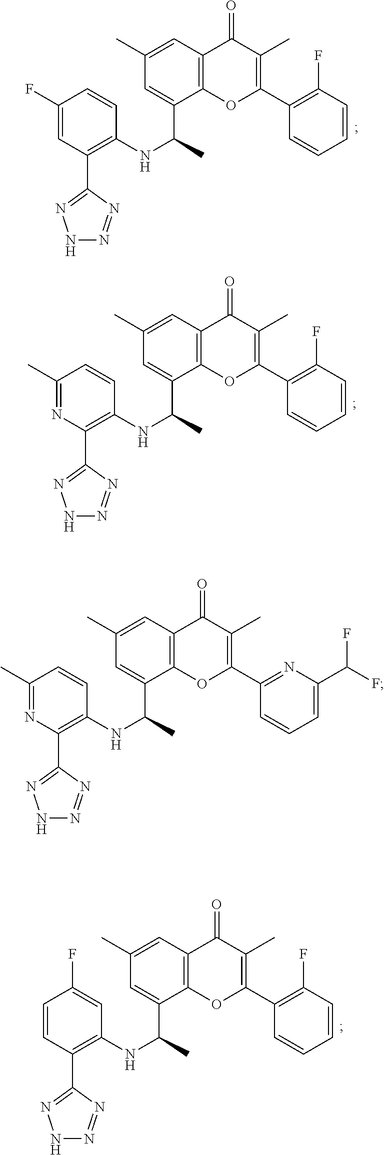 [SMILES: Cc1cc([C@@H](C)Nc2ccc(F)cc2-c2nn[nH]n2)c2oc(-c3ccccc3F)c(C)c(=O)c2c1 ; Cc1cc([C@@H](C)Nc2ccc(C)nc2-c2nn[nH]n2)c2oc(-c3ccccc3F)c(C)c(=O)c2c1 ; Cc1cc([C@@H](C)Nc2ccc(C)nc2-c2nn[nH]n2)c2oc(-c3cccc(C(F)F)n3)c(C)c(=O)c2c1 ; Cc1cc([C@@H](C)Nc2cc(F)ccc2-c2nn[nH]n2)c2oc(-c3ccccc3F)c(C)c(=O)c2c1] 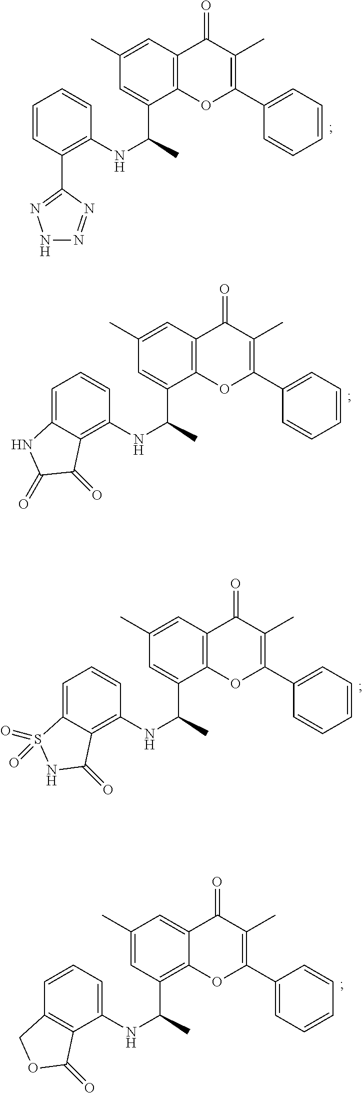 [SMILES: Cc1cc([C@@H](C)Nc2ccccc2-c2nn[nH]n2)c2oc(-c3ccccc3)c(C)c(=O)c2c1 ; Cc1cc([C@@H](C)Nc2cccc3c2C(=O)C(=O)N3)c2oc(-c3ccccc3)c(C)c(=O)c2c1 ; Cc1cc([C@@H](C)Nc2cccc3c2C(=O)NS3(=O)=O)c2oc(-c3ccccc3)c(C)c(=O)c2c1 ; Cc1cc([C@@H](C)Nc2cccc3c2C(=O)OC3)c2oc(-c3ccccc3)c(C)c(=O)c2c1] 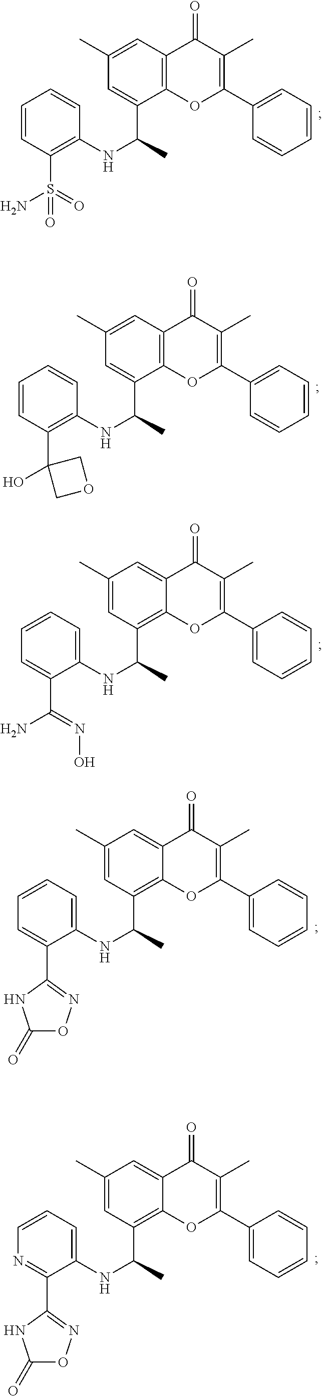 [SMILES: Cc1cc([C@@H](C)Nc2ccccc2S(N)(=O)=O)c2oc(-c3ccccc3)c(C)c(=O)c2c1 ; Cc1cc([C@@H](C)Nc2ccccc2C2(O)COC2)c2oc(-c3ccccc3)c(C)c(=O)c2c1 ; Cc1cc([C@@H](C)Nc2ccccc2/C(N)=N/O)c2oc(-c3ccccc3)c(C)c(=O)c2c1 ; Cc1cc([C@@H](C)Nc2ccccc2-c2noc(=O)[nH]2)c2oc(-c3ccccc3)c(C)c(=O)c2c1 ; Cc1cc([C@@H](C)Nc2cccnc2-c2noc(=O)[nH]2)c2oc(-c3ccccc3)c(C)c(=O)c2c1] 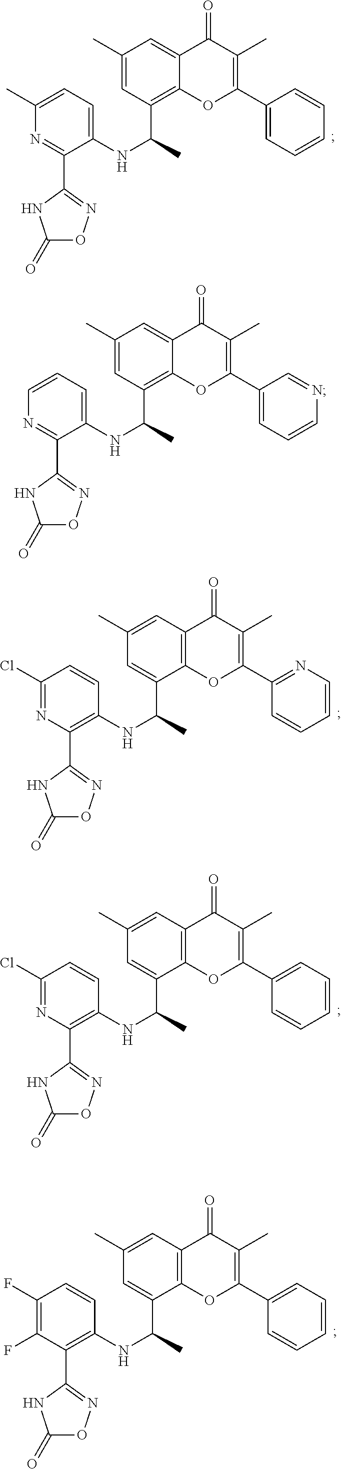 [SMILES: Cc1cc([C@@H](C)Nc2ccc(C)nc2-c2noc(=O)[nH]2)c2oc(-c3ccccc3)c(C)c(=O)c2c1 ; Cc1cc([C@@H](C)Nc2cccnc2-c2noc(=O)[nH]2)c2oc(-c3cccnc3)c(C)c(=O)c2c1 ; Cc1cc([C@@H](C)Nc2ccc(Cl)nc2-c2noc(=O)[nH]2)c2oc(-c3ccccn3)c(C)c(=O)c2c1 ; Cc1cc([C@@H](C)Nc2ccc(Cl)nc2-c2noc(=O)[nH]2)c2oc(-c3ccccc3)c(C)c(=O)c2c1 ; Cc1cc([C@@H](C)Nc2ccc(F)c(F)c2-c2noc(=O)[nH]2)c2oc(-c3ccccc3)c(C)c(=O)c2c1] 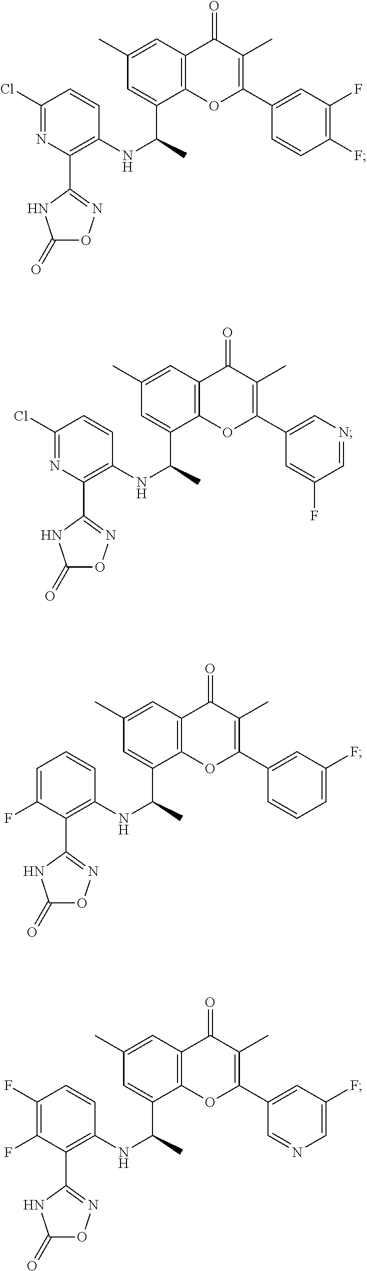 [SMILES: Cc1cc([C@@H](C)Nc2ccc(Cl)nc2-c2noc(=O)[nH]2)c2oc(-c3ccc(F)c(F)c3)c(C)c(=O)c2c1 ; Cc1cc([C@@H](C)Nc2ccc(Cl)nc2-c2noc(=O)[nH]2)c2oc(-c3cncc(F)c3)c(C)c(=O)c2c1 ; Cc1cc([C@@H](C)Nc2cccc(F)c2-c2noc(=O)[nH]2)c2oc(-c3cccc(F)c3)c(C)c(=O)c2c1 ; Cc1cc([C@@H](C)Nc2ccc(F)c(F)c2-c2noc(=O)[nH]2)c2oc(-c3cncc(F)c3)c(C)c(=O)c2c1] 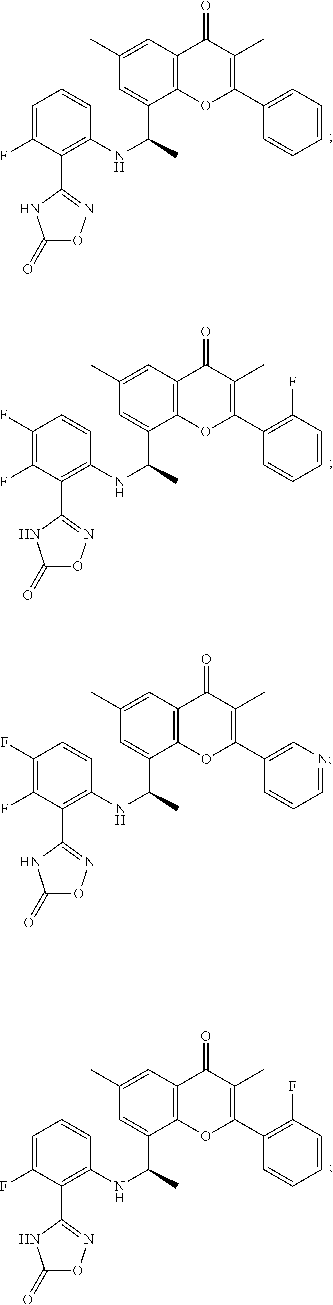 [SMILES: Cc1cc([C@@H](C)Nc2cccc(F)c2-c2noc(=O)[nH]2)c2oc(-c3ccccc3)c(C)c(=O)c2c1 ; Cc1cc([C@@H](C)Nc2ccc(F)c(F)c2-c2noc(=O)[nH]2)c2oc(-c3ccccc3F)c(C)c(=O)c2c1 ; Cc1cc([C@@H](C)Nc2ccc(F)c(F)c2-c2noc(=O)[nH]2)c2oc(-c3cccnc3)c(C)c(=O)c2c1 ; Cc1cc([C@@H](C)Nc2cccc(F)c2-c2noc(=O)[nH]2)c2oc(-c3ccccc3F)c(C)c(=O)c2c1] 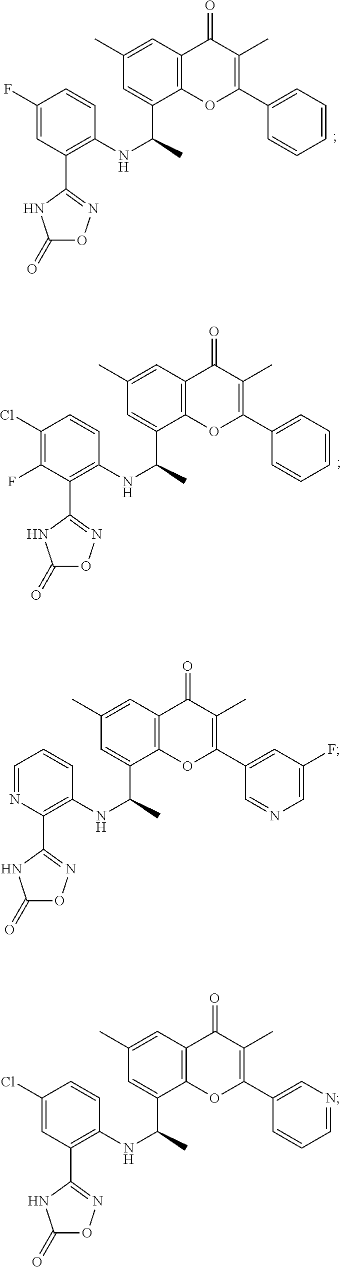 [SMILES: Cc1cc([C@@H](C)Nc2ccc(F)cc2-c2noc(=O)[nH]2)c2oc(-c3ccccc3)c(C)c(=O)c2c1 ; Cc1cc([C@@H](C)Nc2ccc(Cl)c(F)c2-c2noc(=O)[nH]2)c2oc(-c3ccccc3)c(C)c(=O)c2c1 ; Cc1cc([C@@H](C)Nc2cccnc2-c2noc(=O)[nH]2)c2oc(-c3cncc(F)c3)c(C)c(=O)c2c1 ; Cc1cc([C@@H](C)Nc2ccc(Cl)cc2-c2noc(=O)[nH]2)c2oc(-c3cccnc3)c(C)c(=O)c2c1] 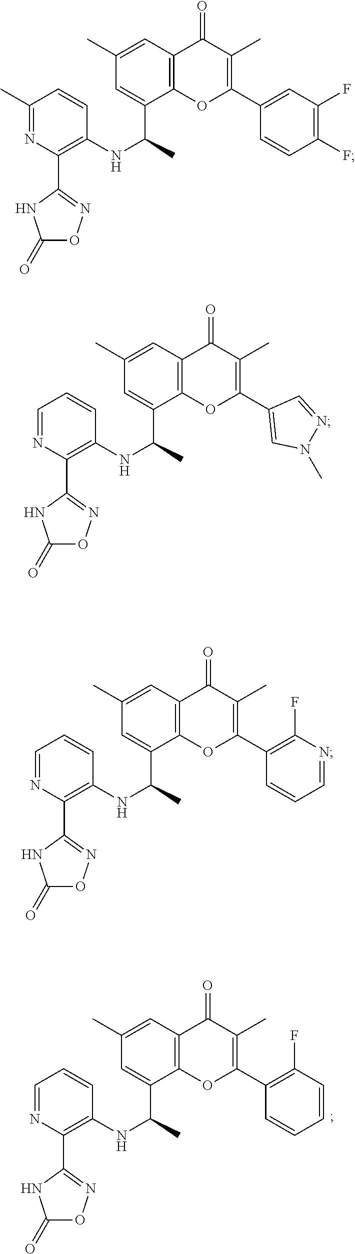 [SMILES: Cc1cc([C@@H](C)Nc2ccc(C)nc2-c2noc(=O)[nH]2)c2oc(-c3ccc(F)c(F)c3)c(C)c(=O)c2c1 ; Cc1cc([C@@H](C)Nc2cccnc2-c2noc(=O)[nH]2)c2oc(-c3cnn(C)c3)c(C)c(=O)c2c1 ; Cc1cc([C@@H](C)Nc2cccnc2-c2noc(=O)[nH]2)c2oc(-c3cccnc3F)c(C)c(=O)c2c1 ; Cc1cc([C@@H](C)Nc2cccnc2-c2noc(=O)[nH]2)c2oc(-c3ccccc3F)c(C)c(=O)c2c1] 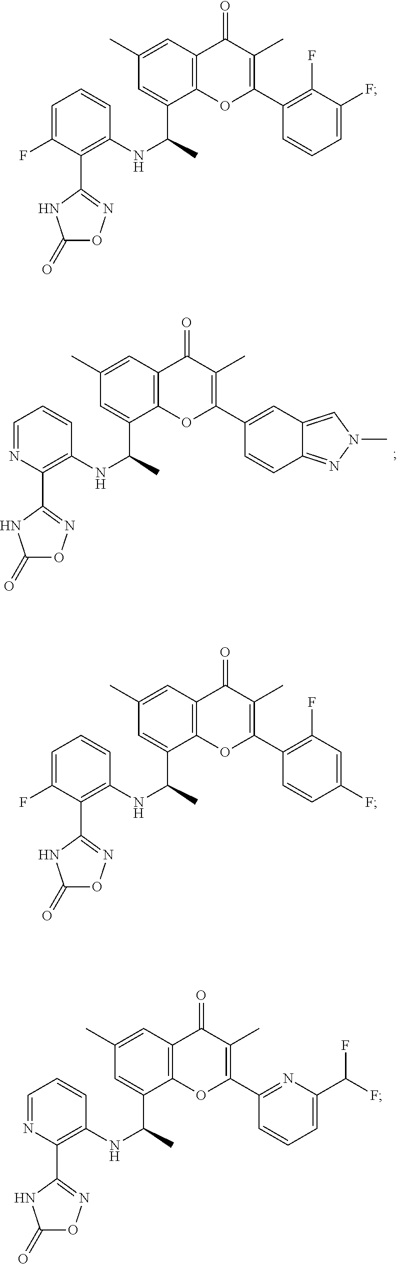 [SMILES: Cc1cc([C@@H](C)Nc2cccc(F)c2-c2noc(=O)[nH]2)c2oc(-c3cccc(F)c3F)c(C)c(=O)c2c1 ; Cc1cc([C@@H](C)Nc2cccnc2-c2noc(=O)[nH]2)c2oc(-c3ccc4nn(C)cc4c3)c(C)c(=O)c2c1 ; Cc1cc([C@@H](C)Nc2cccc(F)c2-c2noc(=O)[nH]2)c2oc(-c3ccc(F)cc3F)c(C)c(=O)c2c1 ; Cc1cc([C@@H](C)Nc2cccnc2-c2noc(=O)[nH]2)c2oc(-c3cccc(C(F)F)n3)c(C)c(=O)c2c1] 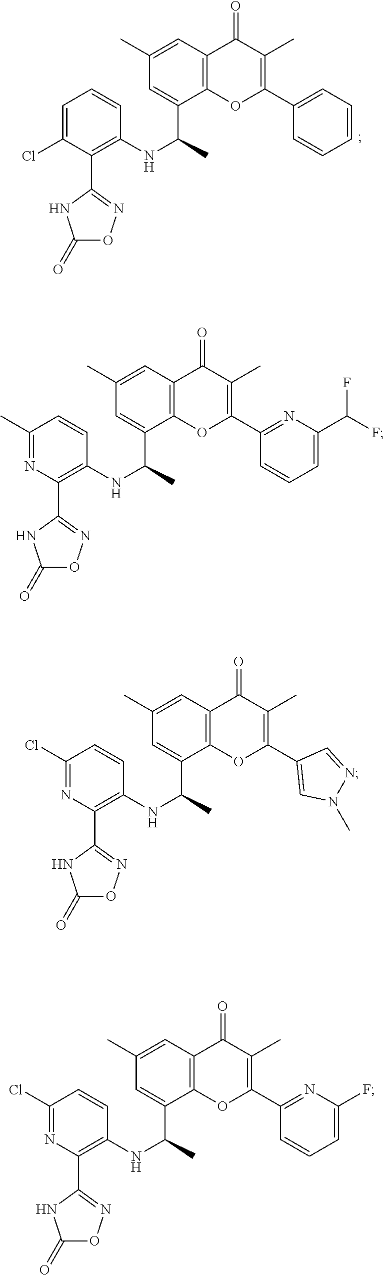 [SMILES: Cc1cc([C@@H](C)Nc2cccc(Cl)c2-c2noc(=O)[nH]2)c2oc(-c3ccccc3)c(C)c(=O)c2c1 ; Cc1cc([C@@H](C)Nc2ccc(C)nc2-c2noc(=O)[nH]2)c2oc(-c3cccc(C(F)F)n3)c(C)c(=O)c2c1 ; Cc1cc([C@@H](C)Nc2ccc(Cl)nc2-c2noc(=O)[nH]2)c2oc(-c3cnn(C)c3)c(C)c(=O)c2c1 ; Cc1cc([C@@H](C)Nc2ccc(Cl)nc2-c2noc(=O)[nH]2)c2oc(-c3cccc(F)n3)c(C)c(=O)c2c1] 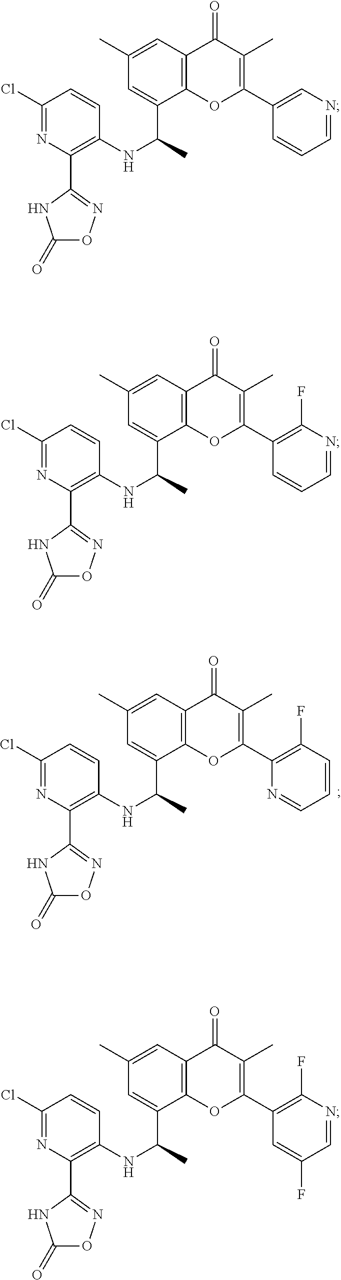 [SMILES: Cc1cc([C@@H](C)Nc2ccc(Cl)nc2-c2noc(=O)[nH]2)c2oc(-c3cccnc3)c(C)c(=O)c2c1 ; Cc1cc([C@@H](C)Nc2ccc(Cl)nc2-c2noc(=O)[nH]2)c2oc(-c3cccnc3F)c(C)c(=O)c2c1 ; Cc1cc([C@@H](C)Nc2ccc(Cl)nc2-c2noc(=O)[nH]2)c2oc(-c3ncccc3F)c(C)c(=O)c2c1 ; Cc1cc([C@@H](C)Nc2ccc(Cl)nc2-c2noc(=O)[nH]2)c2oc(-c3cc(F)cnc3F)c(C)c(=O)c2c1] 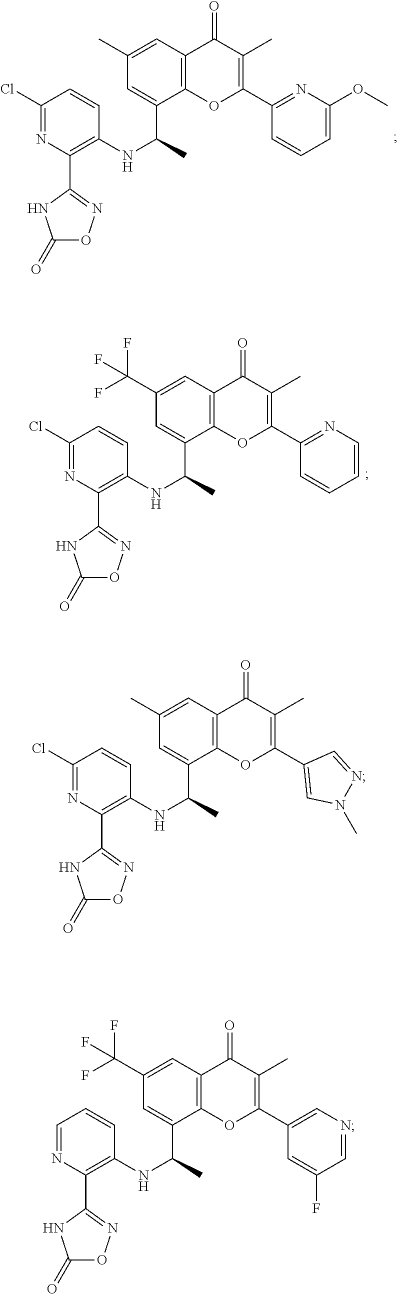 [SMILES: COc1cccc(-c2oc3c([C@@H](C)Nc4ccc(Cl)nc4-c4noc(=O)[nH]4)cc(C)cc3c(=O)c2C)n1 ; Cc1c(-c2ccccn2)oc2c([C@@H](C)Nc3ccc(Cl)nc3-c3noc(=O)[nH]3)cc(C(F)(F)F)cc2c1=O ; Cc1cc([C@@H](C)Nc2ccc(Cl)nc2-c2noc(=O)[nH]2)c2oc(-c3cnn(C)c3)c(C)c(=O)c2c1 ; Cc1c(-c2cncc(F)c2)oc2c([C@@H](C)Nc3cccnc3-c3noc(=O)[nH]3)cc(C(F)(F)F)cc2c1=O] 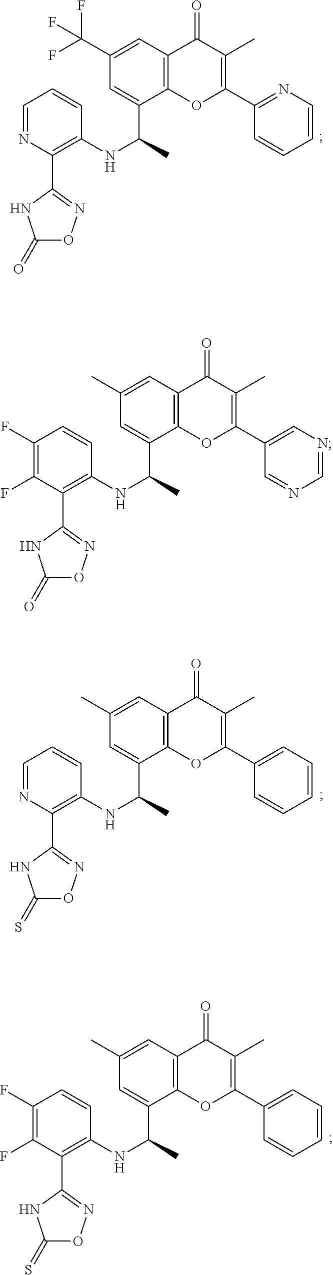 [SMILES: Cc1c(-c2ccccn2)oc2c([C@@H](C)Nc3cccnc3-c3noc(=O)[nH]3)cc(C(F)(F)F)cc2c1=O ; Cc1cc([C@@H](C)Nc2ccc(F)c(F)c2-c2noc(=O)[nH]2)c2oc(-c3cncnc3)c(C)c(=O)c2c1 ; Cc1cc([C@@H](C)Nc2cccnc2-c2noc(=S)[nH]2)c2oc(-c3ccccc3)c(C)c(=O)c2c1 ; Cc1cc([C@@H](C)Nc2ccc(F)c(F)c2-c2noc(=S)[nH]2)c2oc(-c3ccccc3)c(C)c(=O)c2c1] 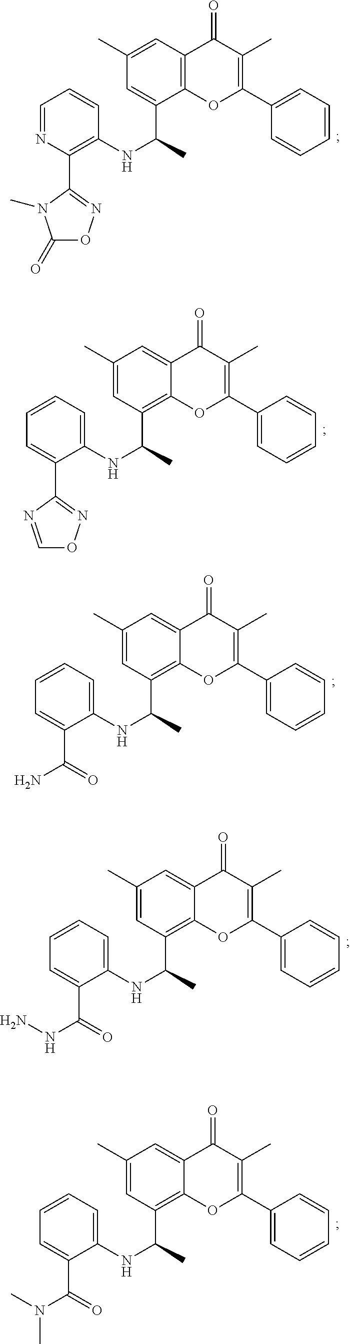 [SMILES: Cc1cc([C@@H](C)Nc2cccnc2-c2noc(=O)n2C)c2oc(-c3ccccc3)c(C)c(=O)c2c1 ; Cc1cc([C@@H](C)Nc2ccccc2-c2ncon2)c2oc(-c3ccccc3)c(C)c(=O)c2c1 ; Cc1cc([C@@H](C)Nc2ccccc2C(N)=O)c2oc(-c3ccccc3)c(C)c(=O)c2c1 ; Cc1cc([C@@H](C)Nc2ccccc2C(=O)NN)c2oc(-c3ccccc3)c(C)c(=O)c2c1 ; Cc1cc([C@@H](C)Nc2ccccc2C(=O)N(C)C)c2oc(-c3ccccc3)c(C)c(=O)c2c1] 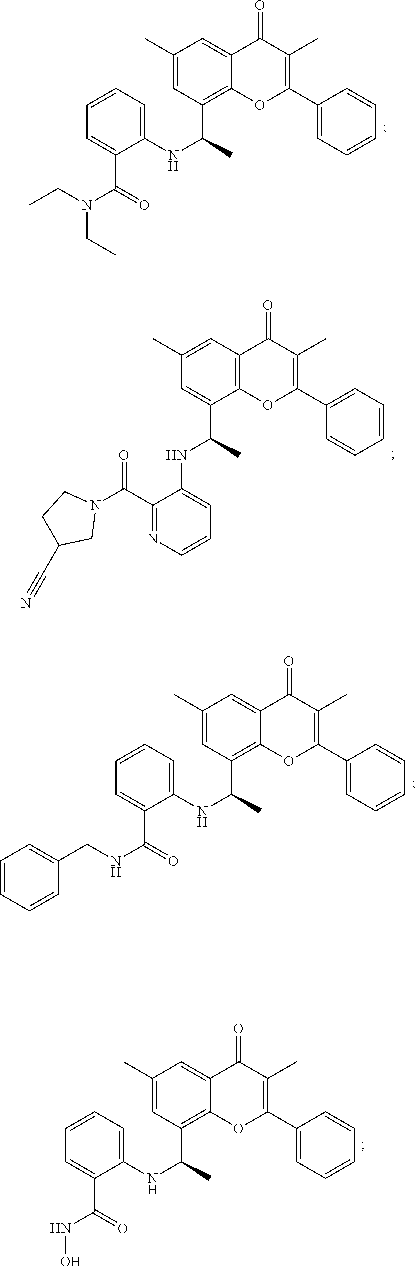 [SMILES: CCN(CC)C(=O)c1ccccc1N[C@H](C)c1cc(C)cc2c(=O)c(C)c(-c3ccccc3)oc12 ; Cc1cc([C@@H](C)Nc2cccnc2C(=O)N2CCC(C#N)C2)c2oc(-c3ccccc3)c(C)c(=O)c2c1 ; Cc1cc([C@@H](C)Nc2ccccc2C(=O)NCc2ccccc2)c2oc(-c3ccccc3)c(C)c(=O)c2c1 ; Cc1cc([C@@H](C)Nc2ccccc2C(=O)NO)c2oc(-c3ccccc3)c(C)c(=O)c2c1] 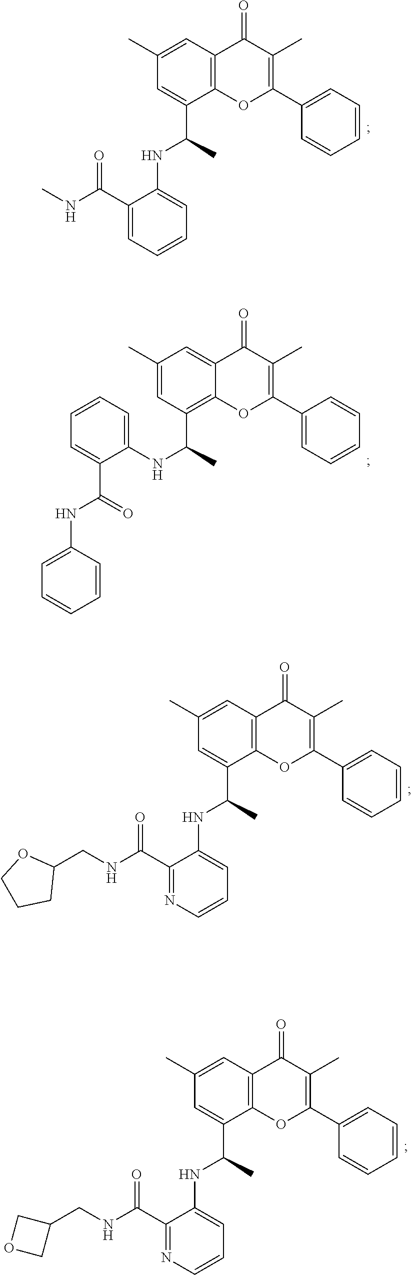 [SMILES: CNC(=O)c1ccccc1N[C@H](C)c1cc(C)cc2c(=O)c(C)c(-c3ccccc3)oc12 ; Cc1cc([C@@H](C)Nc2ccccc2C(=O)Nc2ccccc2)c2oc(-c3ccccc3)c(C)c(=O)c2c1 ; Cc1cc([C@@H](C)Nc2cccnc2C(=O)NCC2CCCO2)c2oc(-c3ccccc3)c(C)c(=O)c2c1 ; Cc1cc([C@@H](C)Nc2cccnc2C(=O)NCC2COC2)c2oc(-c3ccccc3)c(C)c(=O)c2c1] 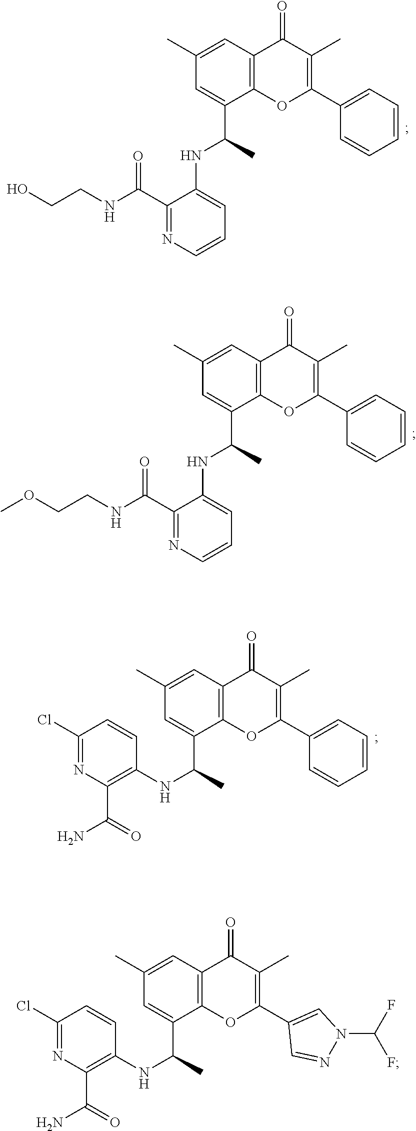 [SMILES: Cc1cc([C@@H](C)Nc2cccnc2C(=O)NCCO)c2oc(-c3ccccc3)c(C)c(=O)c2c1 ; COCCNC(=O)c1ncccc1N[C@H](C)c1cc(C)cc2c(=O)c(C)c(-c3ccccc3)oc12 ; Cc1cc([C@@H](C)Nc2ccc(Cl)nc2C(N)=O)c2oc(-c3ccccc3)c(C)c(=O)c2c1 ; Cc1cc([C@@H](C)Nc2ccc(Cl)nc2C(N)=O)c2oc(-c3cnn(C(F)F)c3)c(C)c(=O)c2c1] 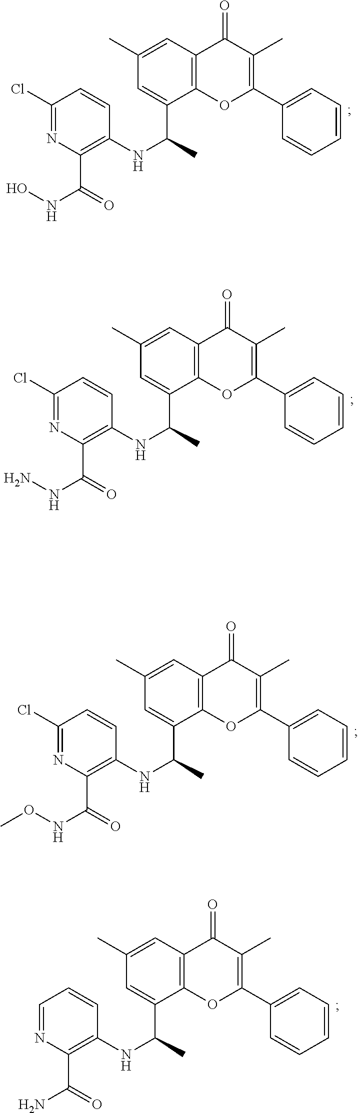 [SMILES: Cc1cc([C@@H](C)Nc2ccc(Cl)nc2C(=O)NO)c2oc(-c3ccccc3)c(C)c(=O)c2c1 ; Cc1cc([C@@H](C)Nc2ccc(Cl)nc2C(=O)NN)c2oc(-c3ccccc3)c(C)c(=O)c2c1 ; CONC(=O)c1nc(Cl)ccc1N[C@H](C)c1cc(C)cc2c(=O)c(C)c(-c3ccccc3)oc12 ; Cc1cc([C@@H](C)Nc2cccnc2C(N)=O)c2oc(-c3ccccc3)c(C)c(=O)c2c1] 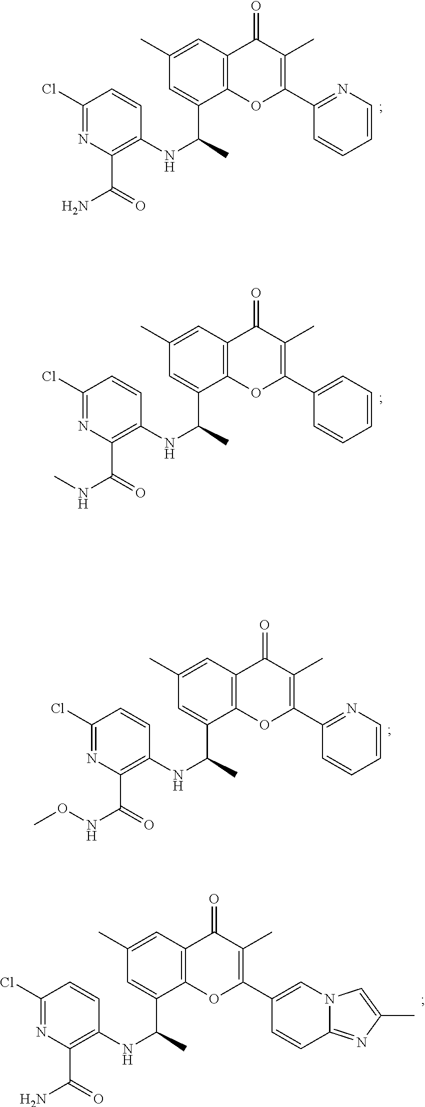 [SMILES: Cc1cc([C@@H](C)Nc2ccc(Cl)nc2C(N)=O)c2oc(-c3ccccn3)c(C)c(=O)c2c1 ; CNC(=O)c1nc(Cl)ccc1N[C@H](C)c1cc(C)cc2c(=O)c(C)c(-c3ccccc3)oc12 ; CONC(=O)c1nc(Cl)ccc1N[C@H](C)c1cc(C)cc2c(=O)c(C)c(-c3ccccn3)oc12 ; Cc1cc([C@@H](C)Nc2ccc(Cl)nc2C(N)=O)c2oc(-c3ccc4nc(C)cn4c3)c(C)c(=O)c2c1] 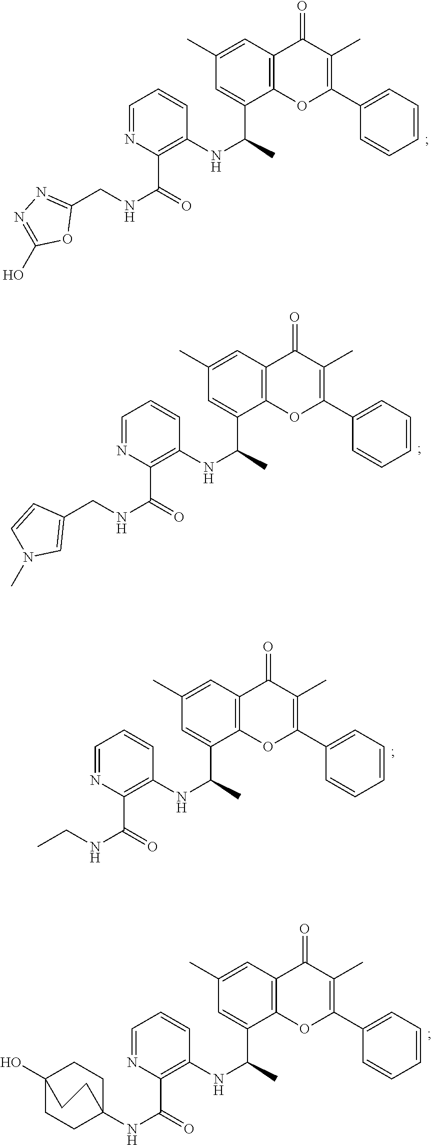 [SMILES: Cc1cc([C@@H](C)Nc2cccnc2C(=O)NCc2nnc(O)o2)c2oc(-c3ccccc3)c(C)c(=O)c2c1 ; Cc1cc([C@@H](C)Nc2cccnc2C(=O)NCc2ccn(C)c2)c2oc(-c3ccccc3)c(C)c(=O)c2c1 ; CCNC(=O)c1ncccc1N[C@H](C)c1cc(C)cc2c(=O)c(C)c(-c3ccccc3)oc12 ; Cc1cc([C@@H](C)Nc2cccnc2C(=O)NC23CCC(O)(CC2)CC3)c2oc(-c3ccccc3)c(C)c(=O)c2c1] 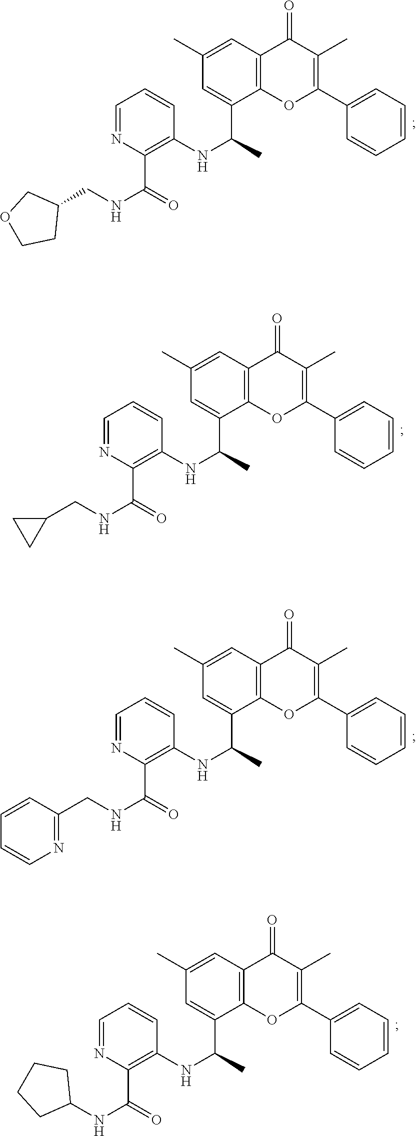 [SMILES: Cc1cc([C@@H](C)Nc2cccnc2C(=O)NC[C@@H]2CCOC2)c2oc(-c3ccccc3)c(C)c(=O)c2c1 ; Cc1cc([C@@H](C)Nc2cccnc2C(=O)NCC2CC2)c2oc(-c3ccccc3)c(C)c(=O)c2c1 ; Cc1cc([C@@H](C)Nc2cccnc2C(=O)NCc2ccccn2)c2oc(-c3ccccc3)c(C)c(=O)c2c1 ; Cc1cc([C@@H](C)Nc2cccnc2C(=O)NC2CCCC2)c2oc(-c3ccccc3)c(C)c(=O)c2c1] 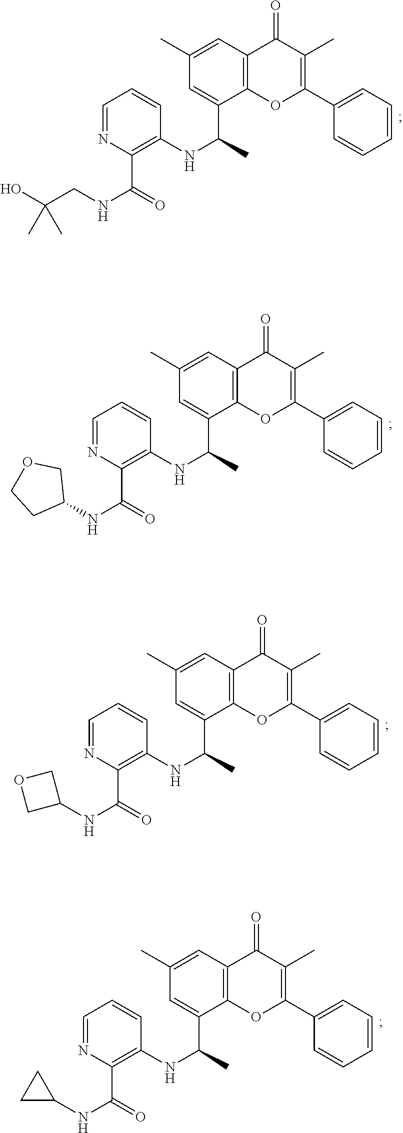 [SMILES: Cc1cc([C@@H](C)Nc2cccnc2C(=O)NCC(C)(C)O)c2oc(-c3ccccc3)c(C)c(=O)c2c1 ; Cc1cc([C@@H](C)Nc2cccnc2C(=O)N[C@@H]2CCOC2)c2oc(-c3ccccc3)c(C)c(=O)c2c1 ; Cc1cc([C@@H](C)Nc2cccnc2C(=O)NC2COC2)c2oc(-c3ccccc3)c(C)c(=O)c2c1 ; Cc1cc([C@@H](C)Nc2cccnc2C(=O)NC2CC2)c2oc(-c3ccccc3)c(C)c(=O)c2c1] 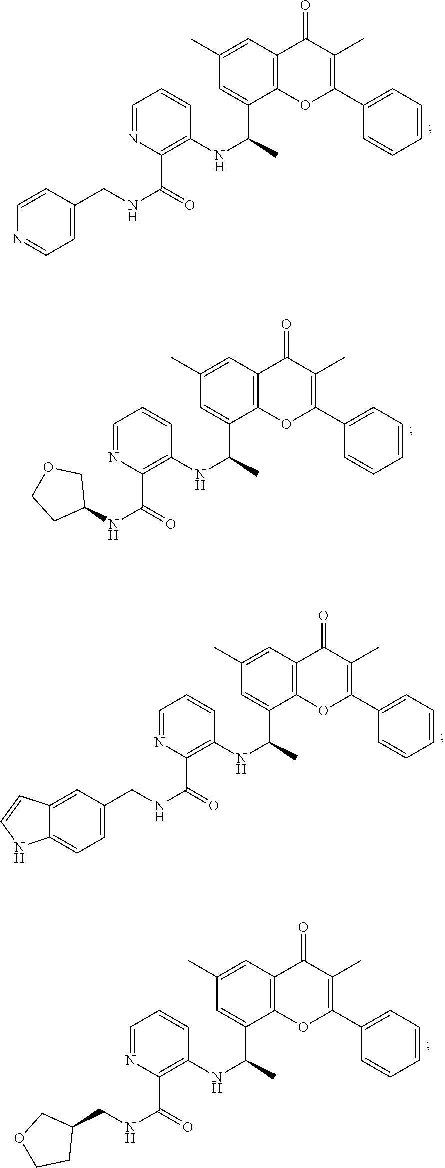 [SMILES: Cc1cc([C@@H](C)Nc2cccnc2C(=O)NCc2ccncc2)c2oc(-c3ccccc3)c(C)c(=O)c2c1 ; Cc1cc([C@@H](C)Nc2cccnc2C(=O)N[C@H]2CCOC2)c2oc(-c3ccccc3)c(C)c(=O)c2c1 ; Cc1cc([C@@H](C)Nc2cccnc2C(=O)NCc2ccc3[nH]ccc3c2)c2oc(-c3ccccc3)c(C)c(=O)c2c1 ; Cc1cc([C@@H](C)Nc2cccnc2C(=O)NC[C@H]2CCOC2)c2oc(-c3ccccc3)c(C)c(=O)c2c1] 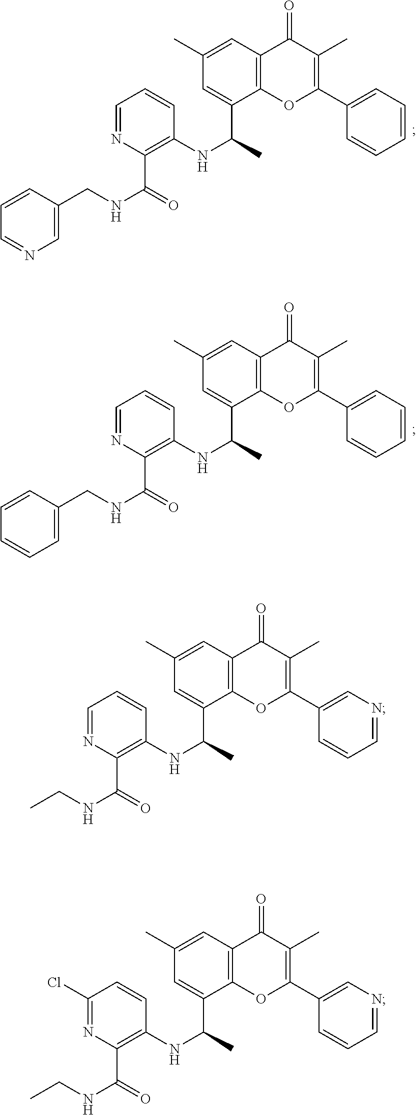 [SMILES: Cc1cc([C@@H](C)Nc2cccnc2C(=O)NCc2cccnc2)c2oc(-c3ccccc3)c(C)c(=O)c2c1 ; Cc1cc([C@@H](C)Nc2cccnc2C(=O)NCc2ccccc2)c2oc(-c3ccccc3)c(C)c(=O)c2c1 ; CCNC(=O)c1ncccc1N[C@H](C)c1cc(C)cc2c(=O)c(C)c(-c3cccnc3)oc12 ; CCNC(=O)c1nc(Cl)ccc1N[C@H](C)c1cc(C)cc2c(=O)c(C)c(-c3cccnc3)oc12] 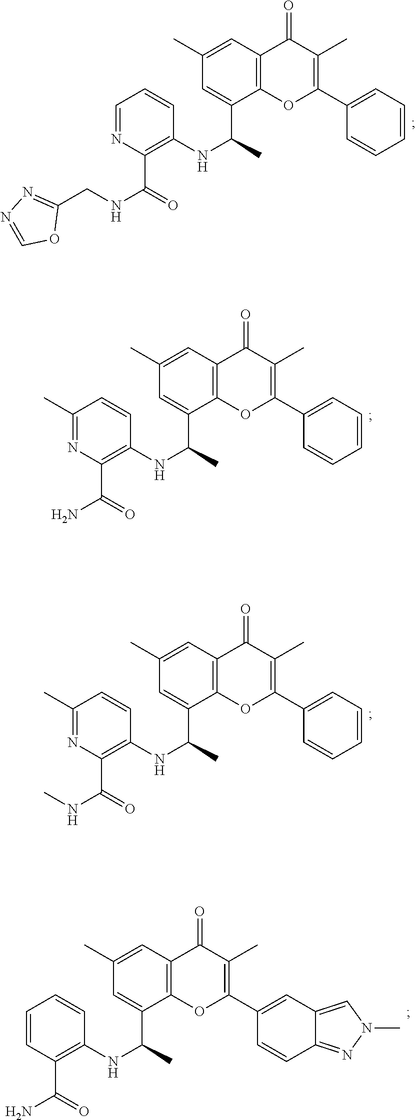 [SMILES: Cc1cc([C@@H](C)Nc2cccnc2C(=O)NCc2nnco2)c2oc(-c3ccccc3)c(C)c(=O)c2c1 ; Cc1cc([C@@H](C)Nc2ccc(C)nc2C(N)=O)c2oc(-c3ccccc3)c(C)c(=O)c2c1 ; CNC(=O)c1nc(C)ccc1N[C@H](C)c1cc(C)cc2c(=O)c(C)c(-c3ccccc3)oc12 ; Cc1cc([C@@H](C)Nc2ccccc2C(N)=O)c2oc(-c3ccc4nn(C)cc4c3)c(C)c(=O)c2c1] 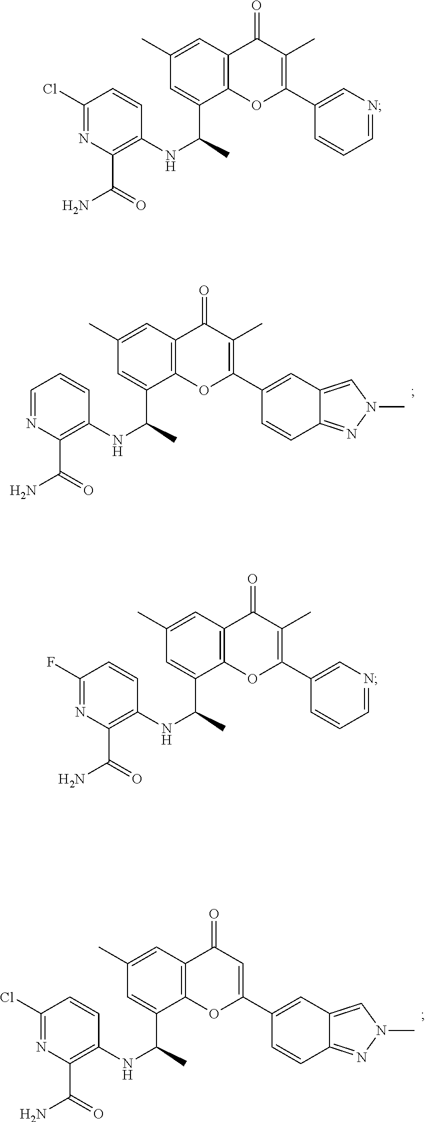 [SMILES: Cc1cc([C@@H](C)Nc2ccc(Cl)nc2C(N)=O)c2oc(-c3cccnc3)c(C)c(=O)c2c1 ; Cc1cc([C@@H](C)Nc2cccnc2C(N)=O)c2oc(-c3ccc4nn(C)cc4c3)c(C)c(=O)c2c1 ; Cc1cc([C@@H](C)Nc2ccc(F)nc2C(N)=O)c2oc(-c3cccnc3)c(C)c(=O)c2c1 ; Cc1cc([C@@H](C)Nc2ccc(Cl)nc2C(N)=O)c2oc(-c3ccc4nn(C)cc4c3)cc(=O)c2c1] 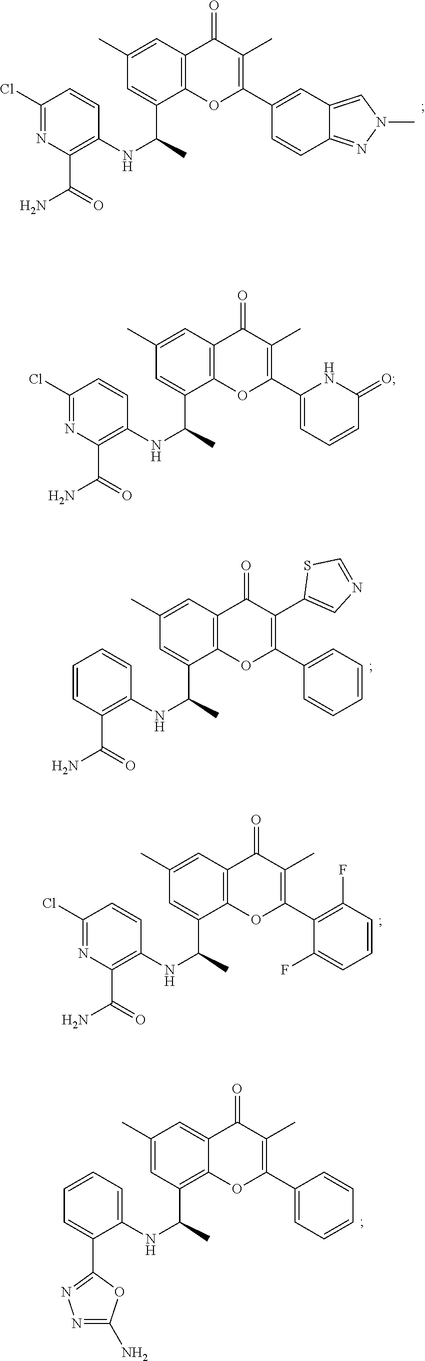 [SMILES: Cc1cc([C@@H](C)Nc2ccc(Cl)nc2C(N)=O)c2oc(-c3ccc4nn(C)cc4c3)c(C)c(=O)c2c1 ; Cc1cc([C@@H](C)Nc2ccc(Cl)nc2C(N)=O)c2oc(-c3cccc(=O)[nH]3)c(C)c(=O)c2c1 ; Cc1cc([C@@H](C)Nc2ccccc2C(N)=O)c2oc(-c3ccccc3)c(-c3cncs3)c(=O)c2c1 ; Cc1cc([C@@H](C)Nc2ccc(Cl)nc2C(N)=O)c2oc(-c3c(F)cccc3F)c(C)c(=O)c2c1 ; Cc1cc([C@@H](C)Nc2ccccc2-c2nnc(N)o2)c2oc(-c3ccccc3)c(C)c(=O)c2c1] 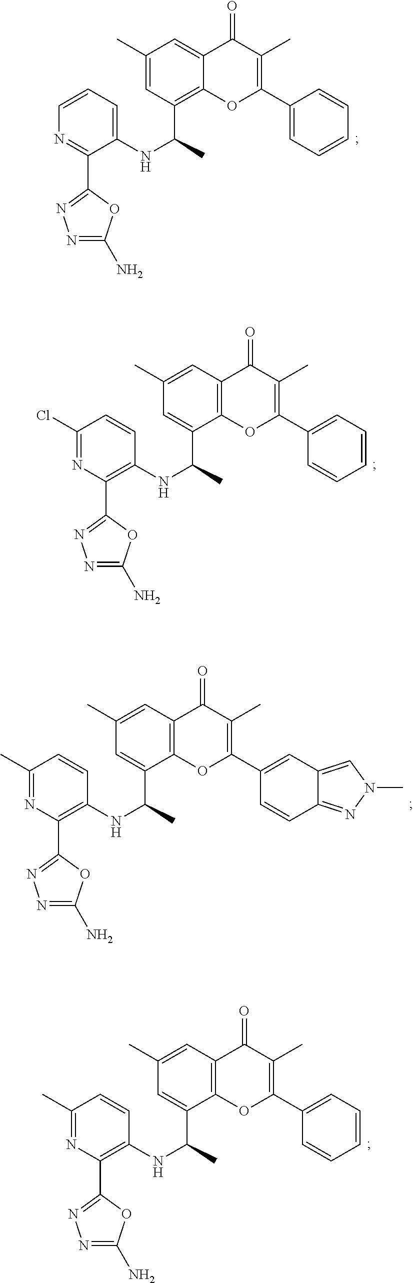 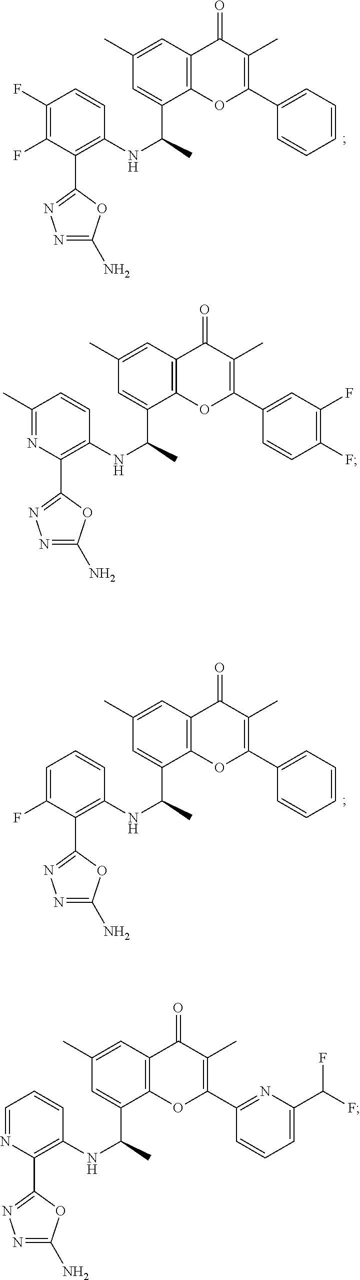 [SMILES: Cc1cc([C@@H](C)Nc2ccc(F)c(F)c2-c2nnc(N)o2)c2oc(-c3ccccc3)c(C)c(=O)c2c1 ; Cc1cc([C@@H](C)Nc2ccc(C)nc2-c2nnc(N)o2)c2oc(-c3ccc(F)c(F)c3)c(C)c(=O)c2c1 ; Cc1cc([C@@H](C)Nc2cccc(F)c2-c2nnc(N)o2)c2oc(-c3ccccc3)c(C)c(=O)c2c1 ; Cc1cc([C@@H](C)Nc2cccnc2-c2nnc(N)o2)c2oc(-c3cccc(C(F)F)n3)c(C)c(=O)c2c1] 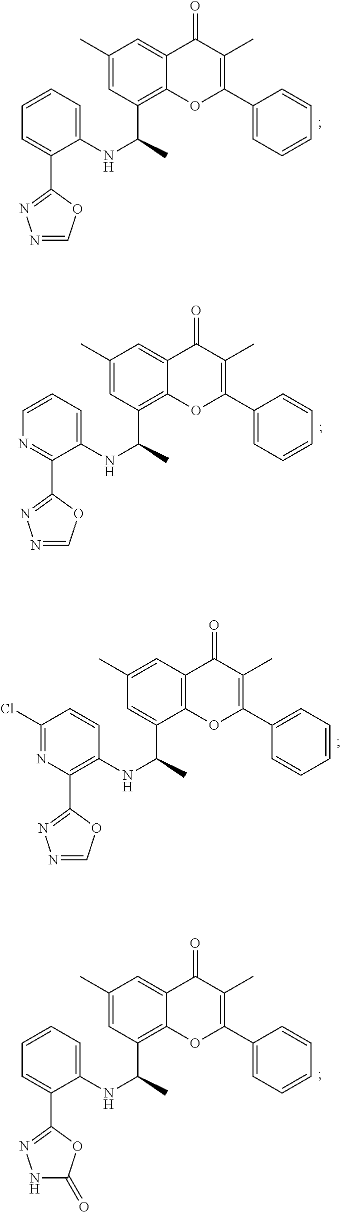 [SMILES: Cc1cc([C@@H](C)Nc2ccccc2-c2nnco2)c2oc(-c3ccccc3)c(C)c(=O)c2c1 ; Cc1cc([C@@H](C)Nc2cccnc2-c2nnco2)c2oc(-c3ccccc3)c(C)c(=O)c2c1 ; Cc1cc([C@@H](C)Nc2ccc(Cl)nc2-c2nnco2)c2oc(-c3ccccc3)c(C)c(=O)c2c1 ; Cc1cc([C@@H](C)Nc2ccccc2-c2n[nH]c(=O)o2)c2oc(-c3ccccc3)c(C)c(=O)c2c1] 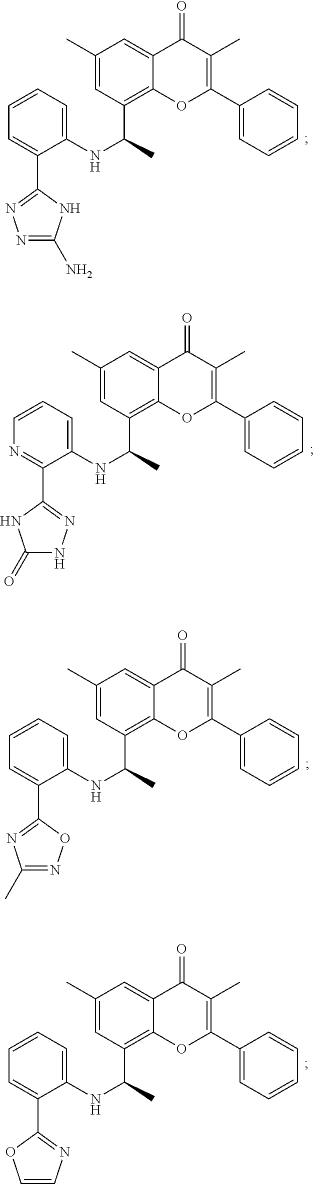 [SMILES: Cc1cc([C@@H](C)Nc2ccccc2-c2nnc(N)[nH]2)c2oc(-c3ccccc3)c(C)c(=O)c2c1 ; Cc1cc([C@@H](C)Nc2cccnc2-c2n[nH]c(=O)[nH]2)c2oc(-c3ccccc3)c(C)c(=O)c2c1 ; Cc1cc([C@@H](C)Nc2ccccc2-c2nc(C)no2)c2oc(-c3ccccc3)c(C)c(=O)c2c1 ; Cc1cc([C@@H](C)Nc2ccccc2-c2ncco2)c2oc(-c3ccccc3)c(C)c(=O)c2c1] 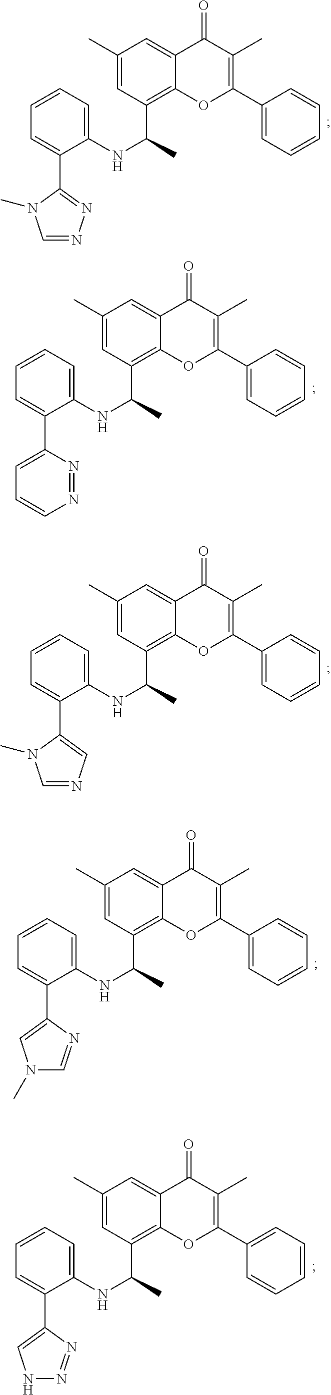 [SMILES: Cc1cc([C@@H](C)Nc2ccccc2-c2nncn2C)c2oc(-c3ccccc3)c(C)c(=O)c2c1 ; Cc1cc([C@@H](C)Nc2ccccc2-c2cccnn2)c2oc(-c3ccccc3)c(C)c(=O)c2c1 ; Cc1cc([C@@H](C)Nc2ccccc2-c2cncn2C)c2oc(-c3ccccc3)c(C)c(=O)c2c1 ; Cc1cc([C@@H](C)Nc2ccccc2-c2cn(C)cn2)c2oc(-c3ccccc3)c(C)c(=O)c2c1 ; Cc1cc([C@@H](C)Nc2ccccc2-c2c[nH]nn2)c2oc(-c3ccccc3)c(C)c(=O)c2c1] 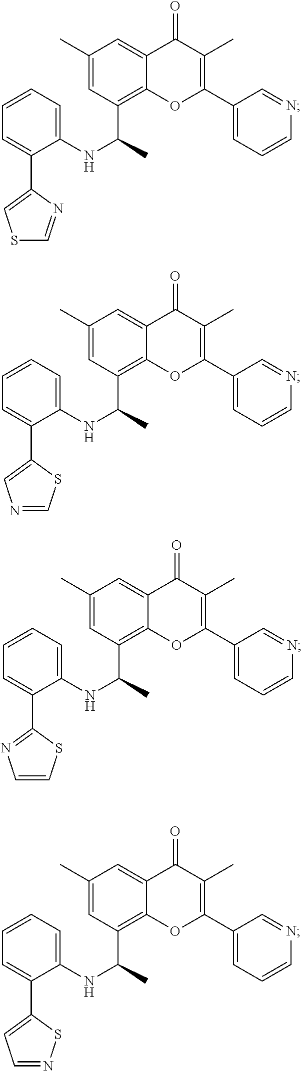 [SMILES: Cc1cc([C@@H](C)Nc2ccccc2-c2cscn2)c2oc(-c3cccnc3)c(C)c(=O)c2c1 ; Cc1cc([C@@H](C)Nc2ccccc2-c2cncs2)c2oc(-c3cccnc3)c(C)c(=O)c2c1 ; Cc1cc([C@@H](C)Nc2ccccc2-c2nccs2)c2oc(-c3cccnc3)c(C)c(=O)c2c1 ; Cc1cc([C@@H](C)Nc2ccccc2-c2ccns2)c2oc(-c3cccnc3)c(C)c(=O)c2c1] 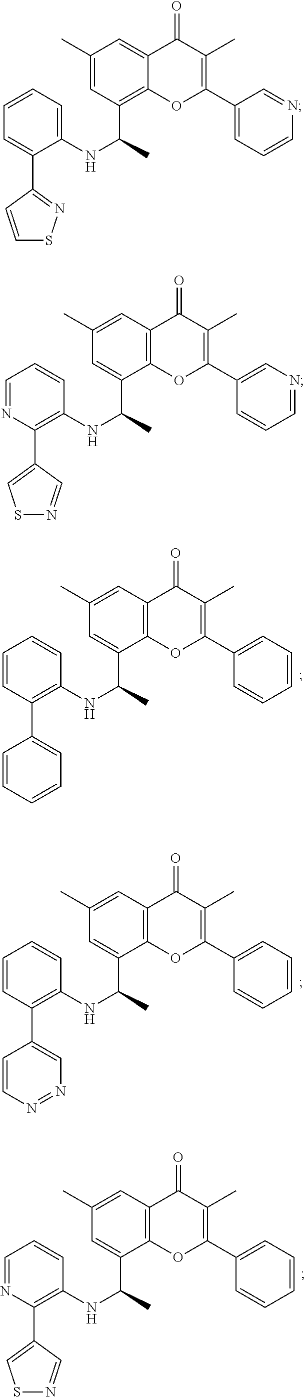 [SMILES: Cc1cc([C@@H](C)Nc2ccccc2-c2ccsn2)c2oc(-c3cccnc3)c(C)c(=O)c2c1 ; Cc1cc([C@@H](C)Nc2cccnc2-c2cnsc2)c2oc(-c3cccnc3)c(C)c(=O)c2c1 ; Cc1cc([C@@H](C)Nc2ccccc2-c2ccccc2)c2oc(-c3ccccc3)c(C)c(=O)c2c1 ; Cc1cc([C@@H](C)Nc2ccccc2-c2ccnnc2)c2oc(-c3ccccc3)c(C)c(=O)c2c1 ; Cc1cc([C@@H](C)Nc2cccnc2-c2cnsc2)c2oc(-c3ccccc3)c(C)c(=O)c2c1] 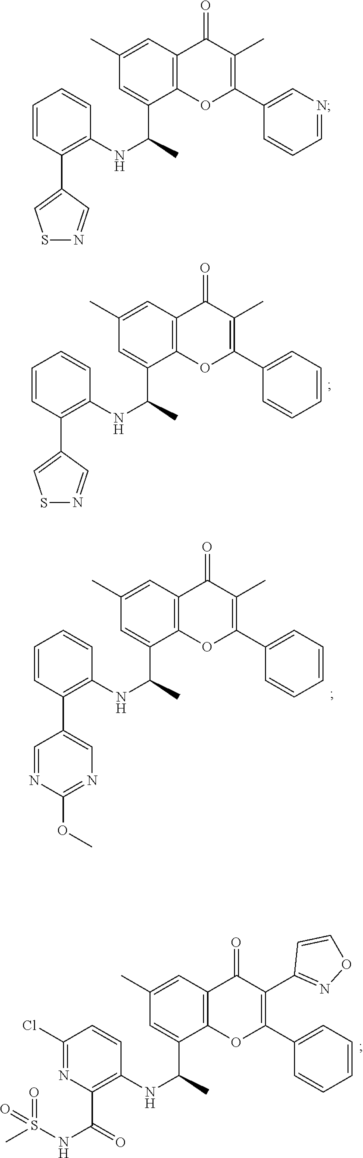 [SMILES: Cc1cc([C@@H](C)Nc2ccccc2-c2cnsc2)c2oc(-c3cccnc3)c(C)c(=O)c2c1 ; Cc1cc([C@@H](C)Nc2ccccc2-c2cnsc2)c2oc(-c3ccccc3)c(C)c(=O)c2c1 ; COc1ncc(-c2ccccc2N[C@H](C)c2cc(C)cc3c(=O)c(C)c(-c4ccccc4)oc23)cn1 ; Cc1cc([C@@H](C)Nc2ccc(Cl)nc2C(=O)NS(C)(=O)=O)c2oc(-c3ccccc3)c(-c3ccon3)c(=O)c2c1] 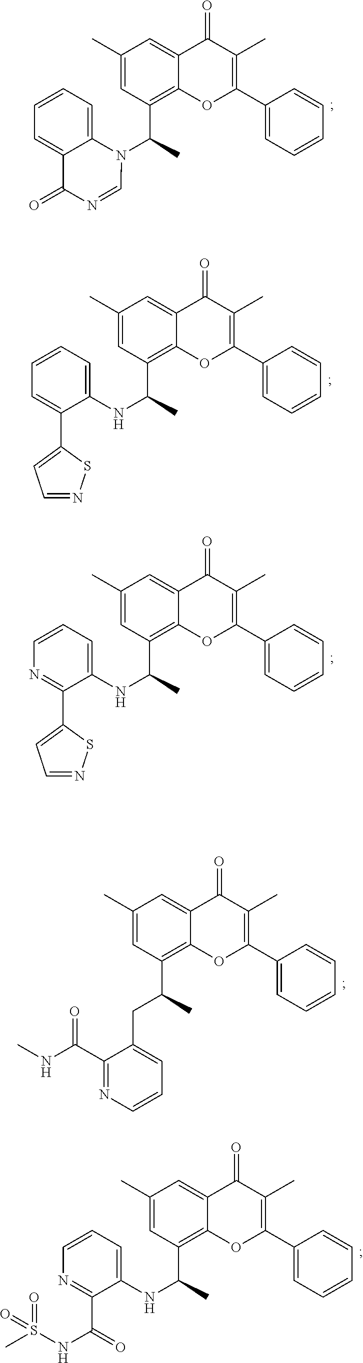 [SMILES: Cc1cc([C@@H](C)n2cnc(=O)c3ccccc32)c2oc(-c3ccccc3)c(C)c(=O)c2c1 ; Cc1cc([C@@H](C)Nc2ccccc2-c2ccns2)c2oc(-c3ccccc3)c(C)c(=O)c2c1 ; Cc1cc([C@@H](C)Nc2cccnc2-c2ccns2)c2oc(-c3ccccc3)c(C)c(=O)c2c1 ; CNC(=O)c1ncccc1C[C@H](C)c1cc(C)cc2c(=O)c(C)c(-c3ccccc3)oc12 ; Cc1cc([C@@H](C)Nc2cccnc2C(=O)NS(C)(=O)=O)c2oc(-c3ccccc3)c(C)c(=O)c2c1] 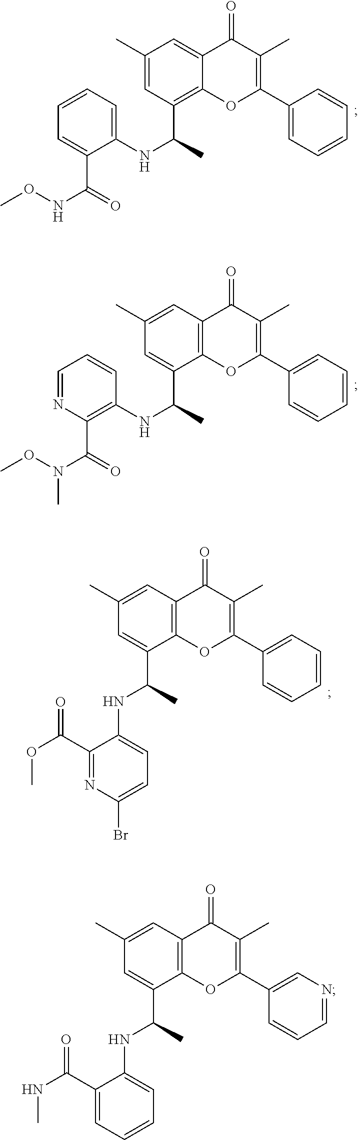 [SMILES: CONC(=O)c1ccccc1N[C@H](C)c1cc(C)cc2c(=O)c(C)c(-c3ccccc3)oc12 ; CON(C)C(=O)c1ncccc1N[C@H](C)c1cc(C)cc2c(=O)c(C)c(-c3ccccc3)oc12 ; COC(=O)c1nc(Br)ccc1N[C@H](C)c1cc(C)cc2c(=O)c(C)c(-c3ccccc3)oc12 ; CNC(=O)c1ccccc1N[C@H](C)c1cc(C)cc2c(=O)c(C)c(-c3cccnc3)oc12] 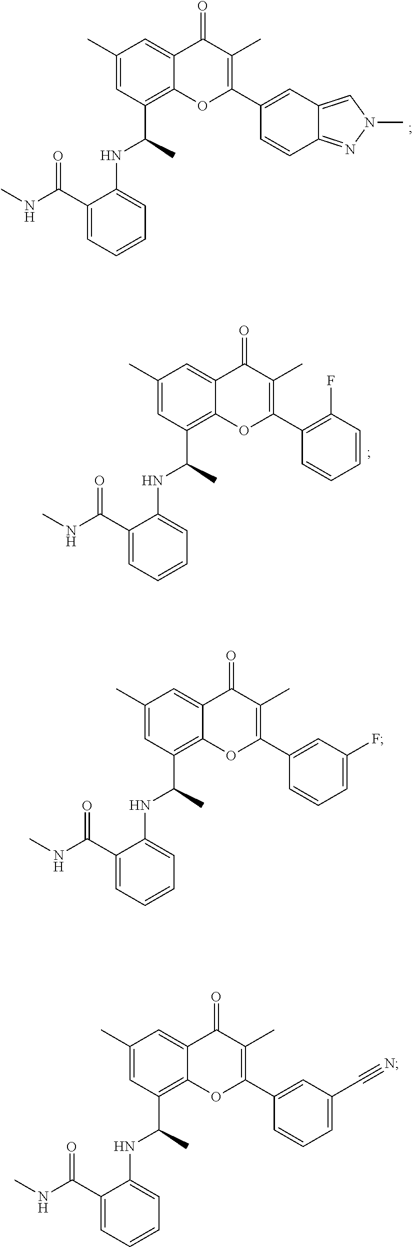 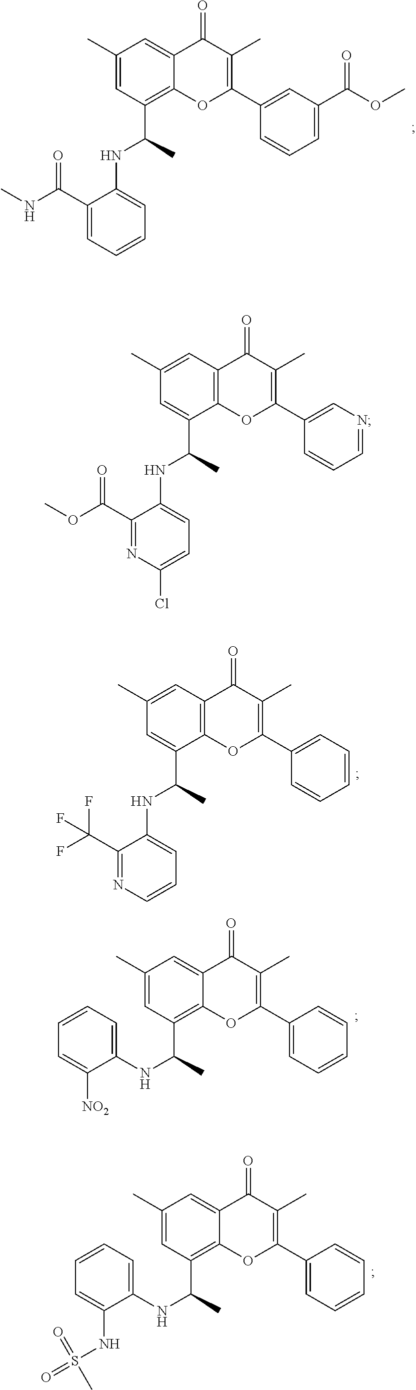 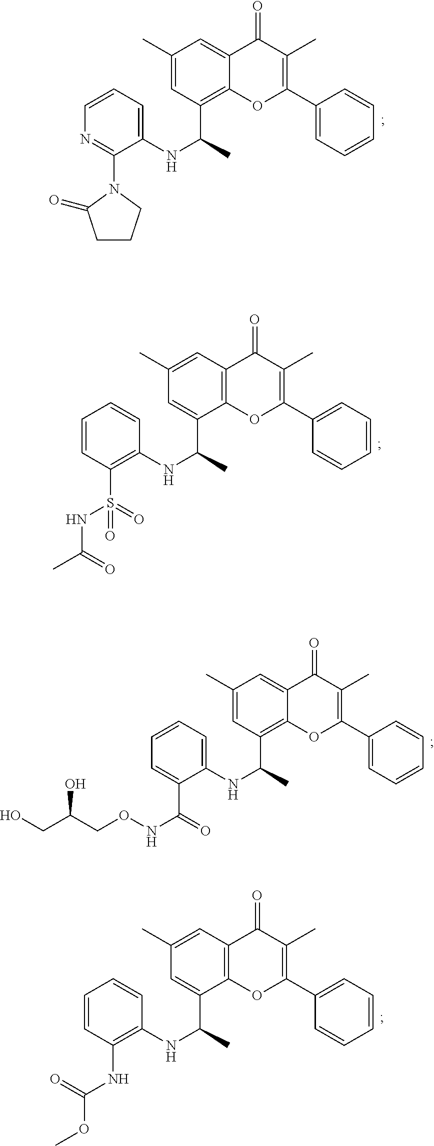 [SMILES: Cc1cc([C@@H](C)Nc2cccnc2N2CCCC2=O)c2oc(-c3ccccc3)c(C)c(=O)c2c1 ; CC(=O)NS(=O)(=O)c1ccccc1N[C@H](C)c1cc(C)cc2c(=O)c(C)c(-c3ccccc3)oc12 ; Cc1cc([C@@H](C)Nc2ccccc2C(=O)NOC[C@H](O)CO)c2oc(-c3ccccc3)c(C)c(=O)c2c1 ; COC(=O)Nc1ccccc1N[C@H](C)c1cc(C)cc2c(=O)c(C)c(-c3ccccc3)oc12] 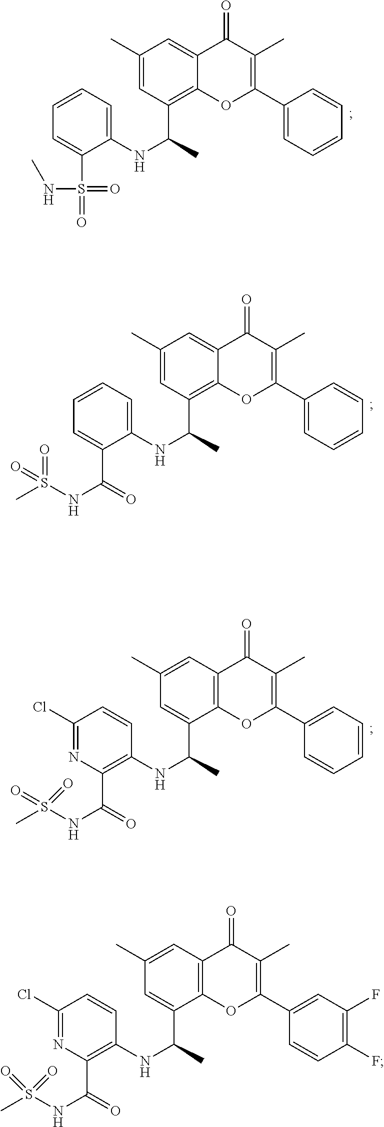 [SMILES: CNS(=O)(=O)c1ccccc1N[C@H](C)c1cc(C)cc2c(=O)c(C)c(-c3ccccc3)oc12 ; Cc1cc([C@@H](C)Nc2ccccc2C(=O)NS(C)(=O)=O)c2oc(-c3ccccc3)c(C)c(=O)c2c1 ; Cc1cc([C@@H](C)Nc2ccc(Cl)nc2C(=O)NS(C)(=O)=O)c2oc(-c3ccccc3)c(C)c(=O)c2c1 ; Cc1cc([C@@H](C)Nc2ccc(Cl)nc2C(=O)NS(C)(=O)=O)c2oc(-c3ccc(F)c(F)c3)c(C)c(=O)c2c1] 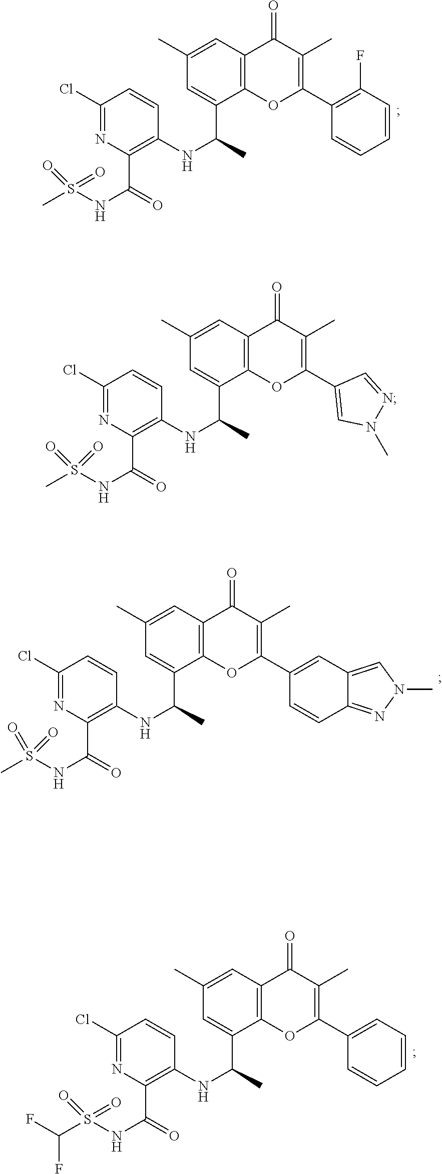 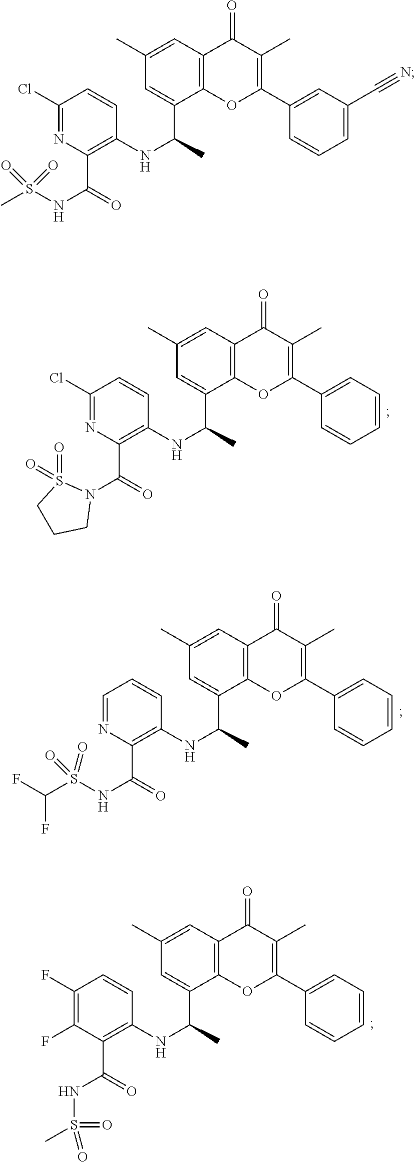 [SMILES: Cc1cc([C@@H](C)Nc2ccc(Cl)nc2C(=O)NS(C)(=O)=O)c2oc(-c3cccc(C#N)c3)c(C)c(=O)c2c1 ; Cc1cc([C@@H](C)Nc2ccc(Cl)nc2C(=O)N2CCCS2(=O)=O)c2oc(-c3ccccc3)c(C)c(=O)c2c1 ; Cc1cc([C@@H](C)Nc2cccnc2C(=O)NS(=O)(=O)C(F)F)c2oc(-c3ccccc3)c(C)c(=O)c2c1 ; Cc1cc([C@@H](C)Nc2ccc(F)c(F)c2C(=O)NS(C)(=O)=O)c2oc(-c3ccccc3)c(C)c(=O)c2c1] 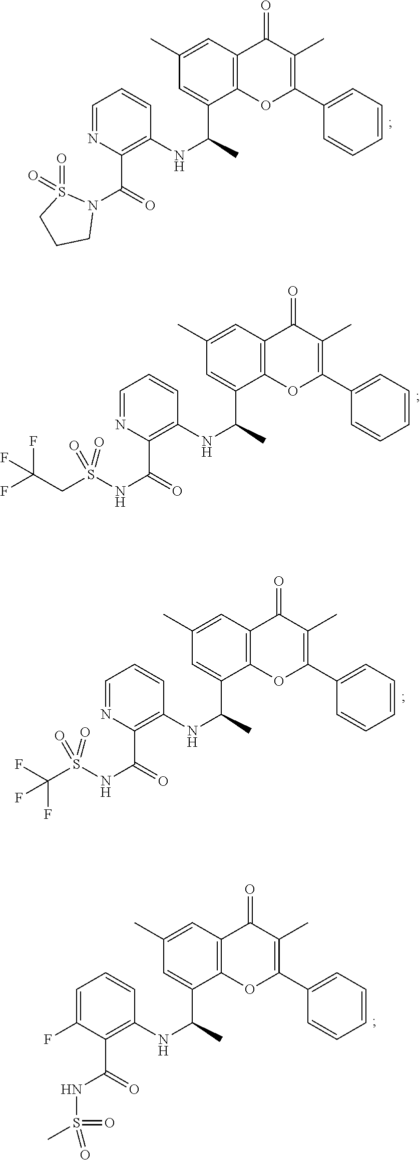 [SMILES: Cc1cc([C@@H](C)Nc2cccnc2C(=O)N2CCCS2(=O)=O)c2oc(-c3ccccc3)c(C)c(=O)c2c1 ; Cc1cc([C@@H](C)Nc2cccnc2C(=O)NS(=O)(=O)CC(F)(F)F)c2oc(-c3ccccc3)c(C)c(=O)c2c1 ; Cc1cc([C@@H](C)Nc2cccnc2C(=O)NS(=O)(=O)C(F)(F)F)c2oc(-c3ccccc3)c(C)c(=O)c2c1 ; Cc1cc([C@@H](C)Nc2cccc(F)c2C(=O)NS(C)(=O)=O)c2oc(-c3ccccc3)c(C)c(=O)c2c1] 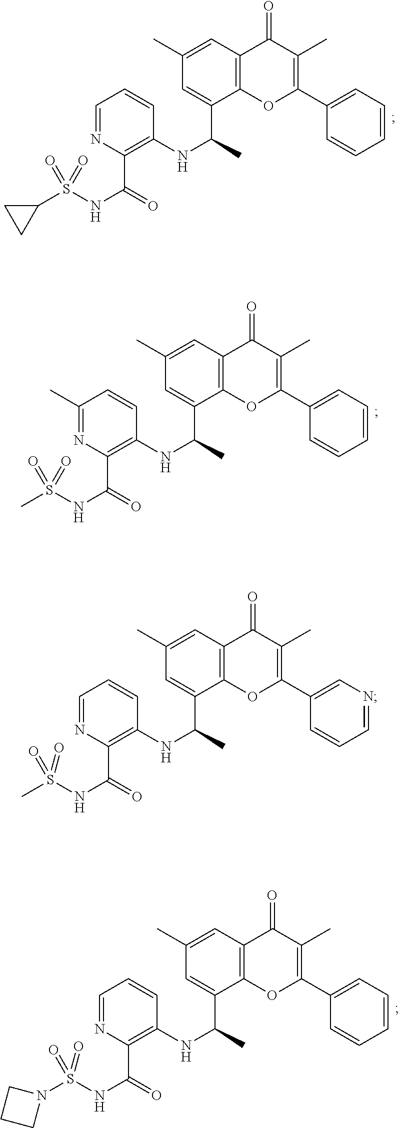 [SMILES: Cc1cc([C@@H](C)Nc2cccnc2C(=O)NS(=O)(=O)C2CC2)c2oc(-c3ccccc3)c(C)c(=O)c2c1 ; Cc1cc([C@@H](C)Nc2ccc(C)nc2C(=O)NS(C)(=O)=O)c2oc(-c3ccccc3)c(C)c(=O)c2c1 ; Cc1cc([C@@H](C)Nc2cccnc2C(=O)NS(C)(=O)=O)c2oc(-c3cccnc3)c(C)c(=O)c2c1 ; Cc1cc([C@@H](C)Nc2cccnc2C(=O)NS(=O)(=O)N2CCC2)c2oc(-c3ccccc3)c(C)c(=O)c2c1] 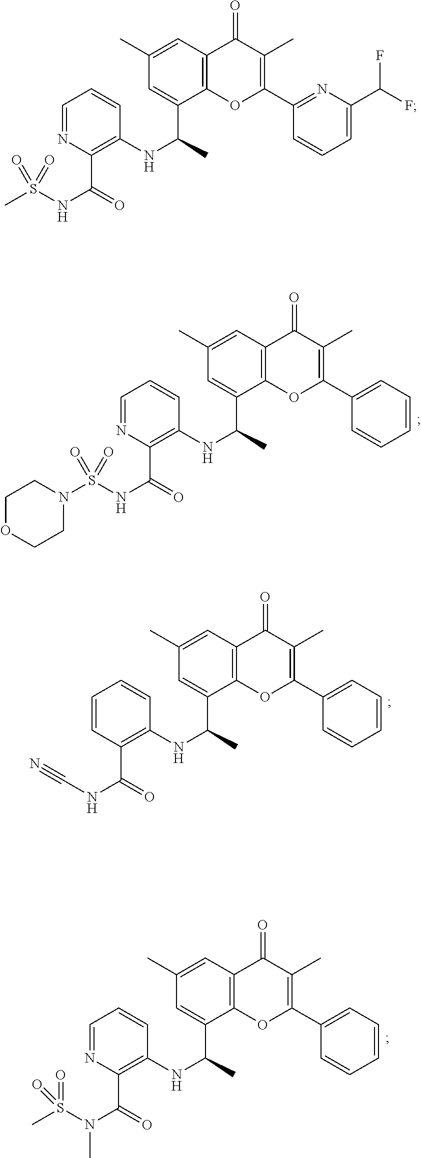 [SMILES: Cc1cc([C@@H](C)Nc2cccnc2C(=O)NS(C)(=O)=O)c2oc(-c3cccc(C(F)F)n3)c(C)c(=O)c2c1 ; Cc1cc([C@@H](C)Nc2cccnc2C(=O)NS(=O)(=O)N2CCOCC2)c2oc(-c3ccccc3)c(C)c(=O)c2c1 ; Cc1cc([C@@H](C)Nc2ccccc2C(=O)NC#N)c2oc(-c3ccccc3)c(C)c(=O)c2c1 ; Cc1cc([C@@H](C)Nc2cccnc2C(=O)N(C)S(C)(=O)=O)c2oc(-c3ccccc3)c(C)c(=O)c2c1] 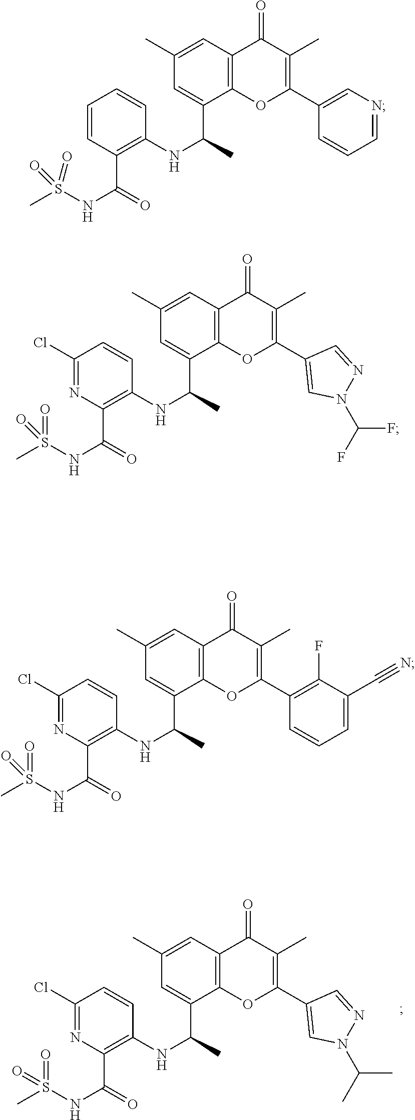 [SMILES: Cc1cc([C@@H](C)Nc2ccccc2C(=O)NS(C)(=O)=O)c2oc(-c3cccnc3)c(C)c(=O)c2c1 ; Cc1cc([C@@H](C)Nc2ccc(Cl)nc2C(=O)NS(C)(=O)=O)c2oc(-c3cnn(C(F)F)c3)c(C)c(=O)c2c1 ; Cc1cc([C@@H](C)Nc2ccc(Cl)nc2C(=O)NS(C)(=O)=O)c2oc(-c3cccc(C#N)c3F)c(C)c(=O)c2c1 ; Cc1cc([C@@H](C)Nc2ccc(Cl)nc2C(=O)NS(C)(=O)=O)c2oc(-c3cnn(C(C)C)c3)c(C)c(=O)c2c1] 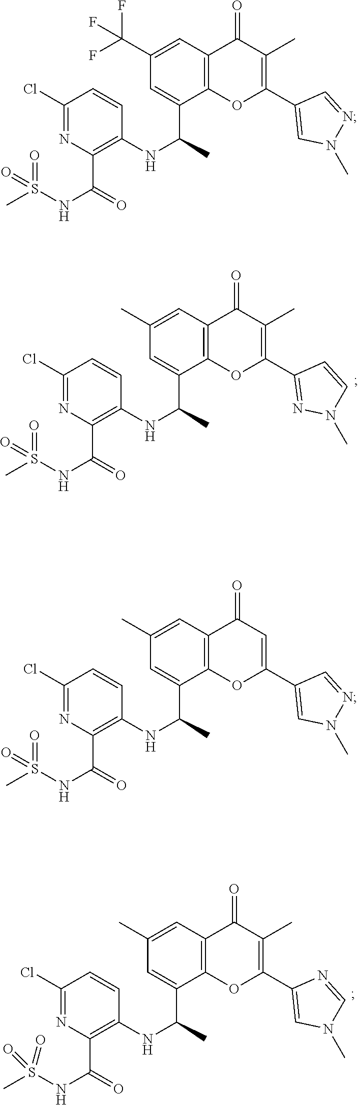 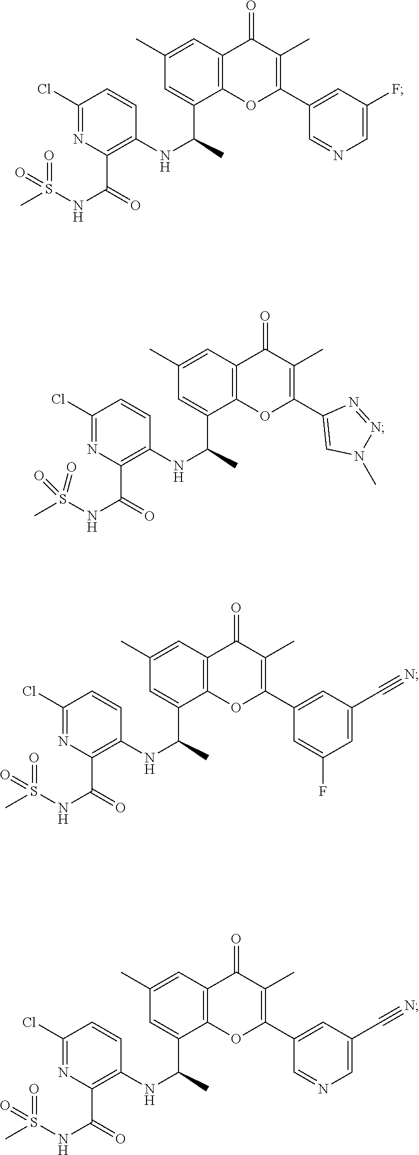 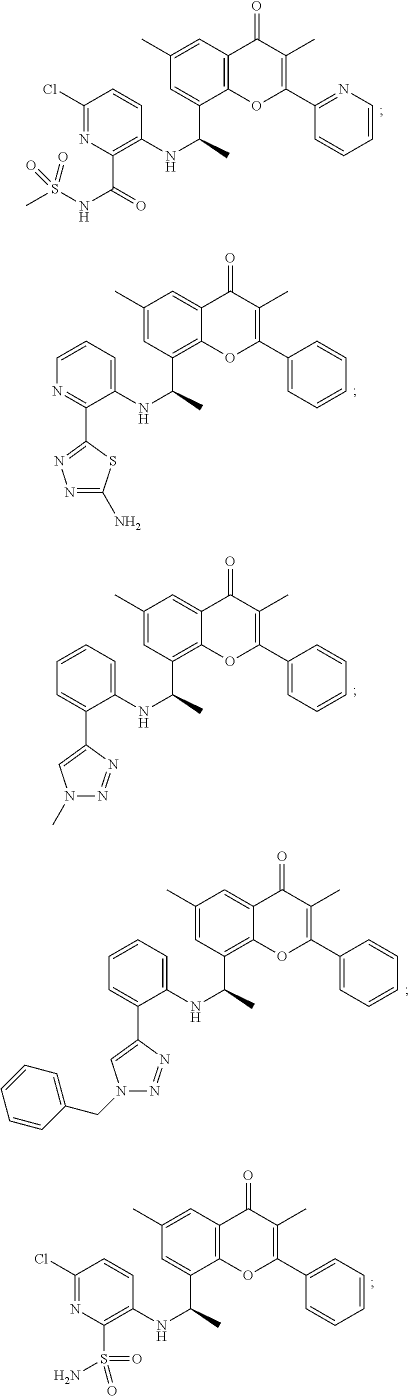 [SMILES: Cc1cc([C@@H](C)Nc2ccc(Cl)nc2C(=O)NS(C)(=O)=O)c2oc(-c3ccccn3)c(C)c(=O)c2c1 ; Cc1cc([C@@H](C)Nc2cccnc2-c2nnc(N)s2)c2oc(-c3ccccc3)c(C)c(=O)c2c1 ; Cc1cc([C@@H](C)Nc2ccccc2-c2cn(C)nn2)c2oc(-c3ccccc3)c(C)c(=O)c2c1 ; Cc1cc([C@@H](C)Nc2ccccc2-c2cn(Cc3ccccc3)nn2)c2oc(-c3ccccc3)c(C)c(=O)c2c1 ; Cc1cc([C@@H](C)Nc2ccc(Cl)nc2S(N)(=O)=O)c2oc(-c3ccccc3)c(C)c(=O)c2c1] 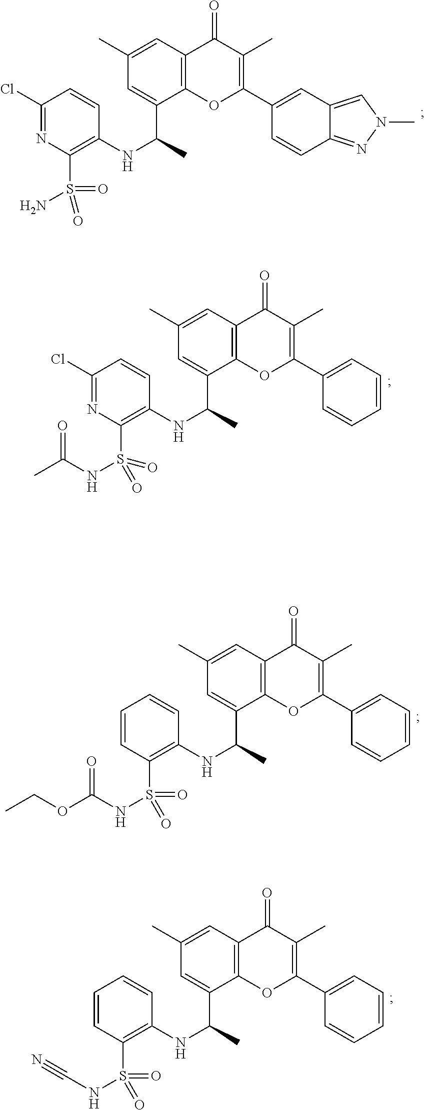 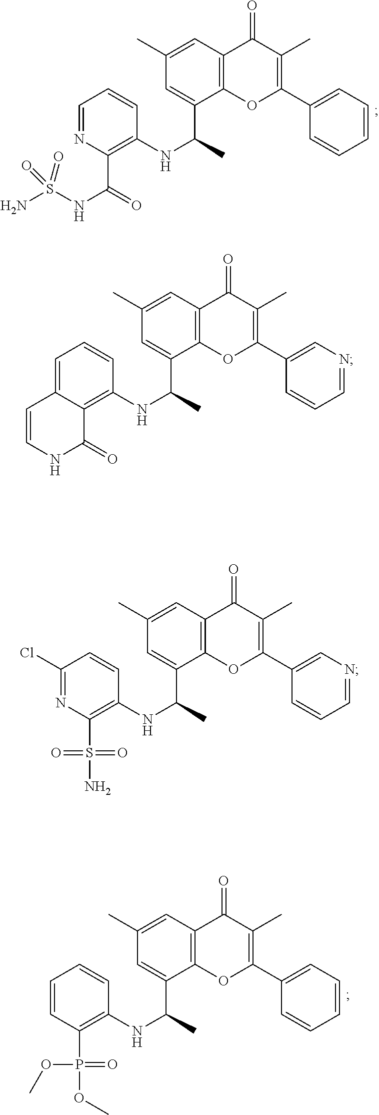 [SMILES: Cc1cc([C@@H](C)Nc2cccnc2C(=O)NS(N)(=O)=O)c2oc(-c3ccccc3)c(C)c(=O)c2c1 ; Cc1cc([C@@H](C)Nc2cccc3cc[nH]c(=O)c23)c2oc(-c3cccnc3)c(C)c(=O)c2c1 ; Cc1cc([C@@H](C)Nc2ccc(Cl)nc2S(N)(=O)=O)c2oc(-c3cccnc3)c(C)c(=O)c2c1 ; COP(=O)(OC)c1ccccc1N[C@H](C)c1cc(C)cc2c(=O)c(C)c(-c3ccccc3)oc12] 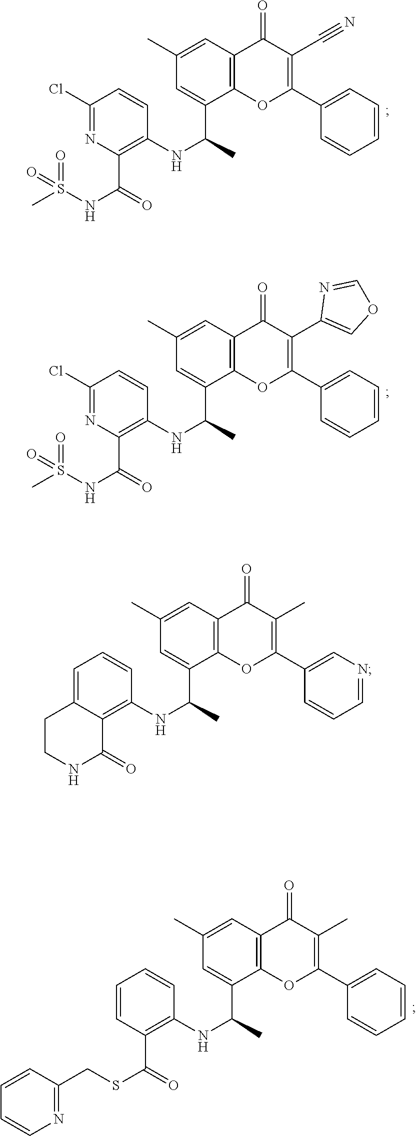 [SMILES: Cc1cc([C@@H](C)Nc2ccc(Cl)nc2C(=O)NS(C)(=O)=O)c2oc(-c3ccccc3)c(C#N)c(=O)c2c1 ; Cc1cc([C@@H](C)Nc2ccc(Cl)nc2C(=O)NS(C)(=O)=O)c2oc(-c3ccccc3)c(-c3cocn3)c(=O)c2c1 ; Cc1cc([C@@H](C)Nc2cccc3c2C(=O)NCC3)c2oc(-c3cccnc3)c(C)c(=O)c2c1 ; Cc1cc([C@@H](C)Nc2ccccc2C(=O)SCc2ccccn2)c2oc(-c3ccccc3)c(C)c(=O)c2c1] 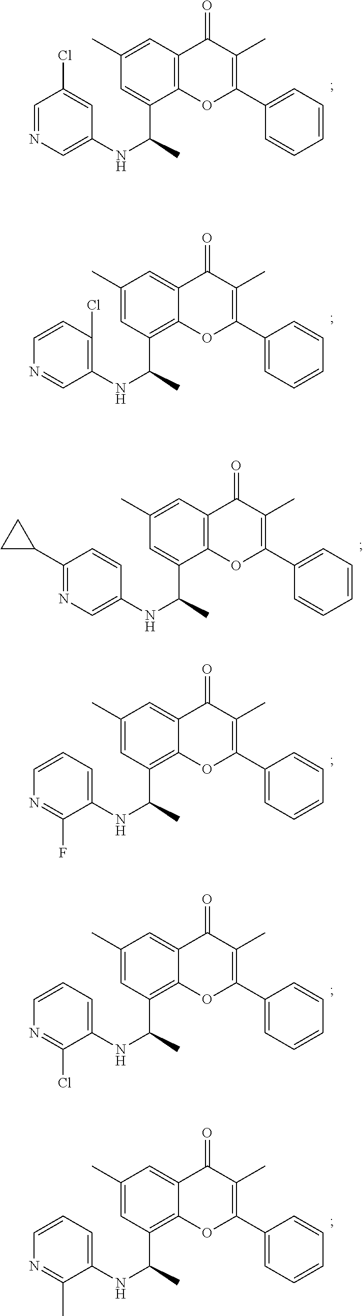 [SMILES: Cc1cc([C@@H](C)Nc2cncc(Cl)c2)c2oc(-c3ccccc3)c(C)c(=O)c2c1 ; Cc1cc([C@@H](C)Nc2cnccc2Cl)c2oc(-c3ccccc3)c(C)c(=O)c2c1 ; Cc1cc([C@@H](C)Nc2ccc(C3CC3)nc2)c2oc(-c3ccccc3)c(C)c(=O)c2c1 ; Cc1cc([C@@H](C)Nc2cccnc2F)c2oc(-c3ccccc3)c(C)c(=O)c2c1 ; Cc1cc([C@@H](C)Nc2cccnc2Cl)c2oc(-c3ccccc3)c(C)c(=O)c2c1 ; Cc1cc([C@@H](C)Nc2cccnc2C)c2oc(-c3ccccc3)c(C)c(=O)c2c1] 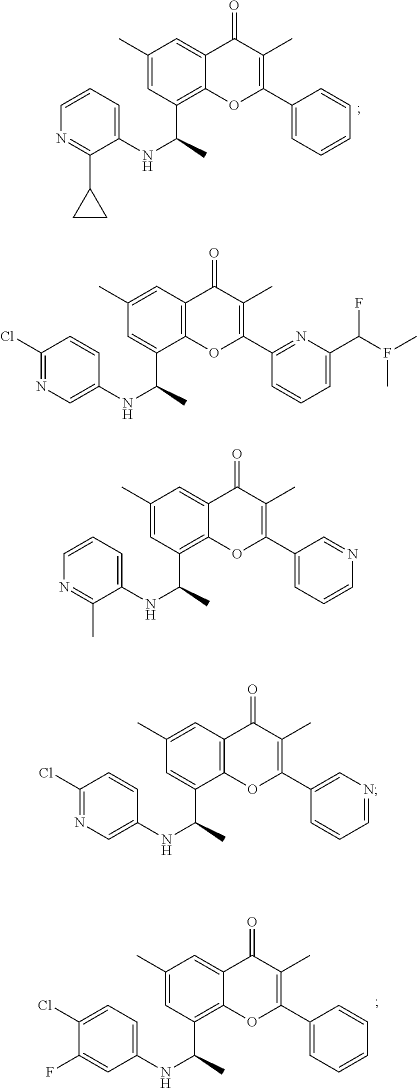 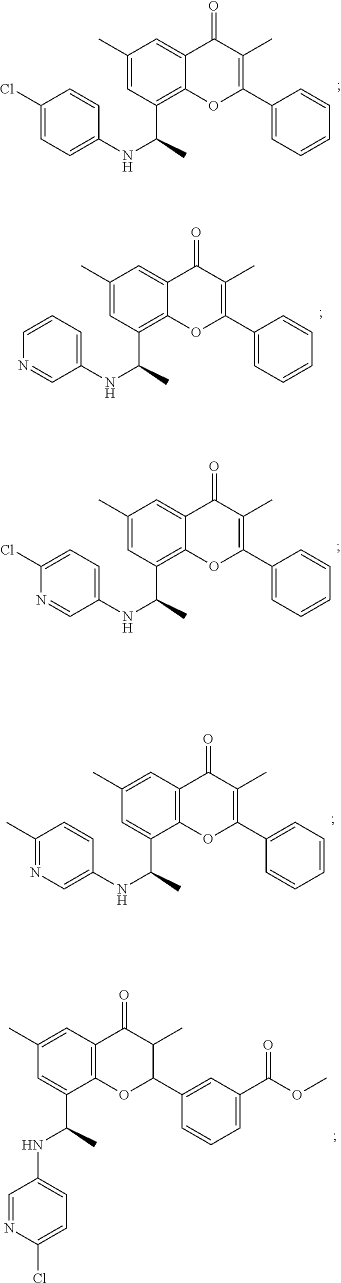 [SMILES: Cc1cc([C@@H](C)Nc2ccc(Cl)cc2)c2oc(-c3ccccc3)c(C)c(=O)c2c1 ; Cc1cc([C@@H](C)Nc2cccnc2)c2oc(-c3ccccc3)c(C)c(=O)c2c1 ; Cc1cc([C@@H](C)Nc2ccc(Cl)nc2)c2oc(-c3ccccc3)c(C)c(=O)c2c1 ; Cc1cc([C@@H](C)Nc2ccc(C)nc2)c2oc(-c3ccccc3)c(C)c(=O)c2c1 ; COC(=O)c1cccc(C2Oc3c(cc(C)cc3[C@@H](C)Nc3ccc(Cl)nc3)C(=O)C2C)c1] 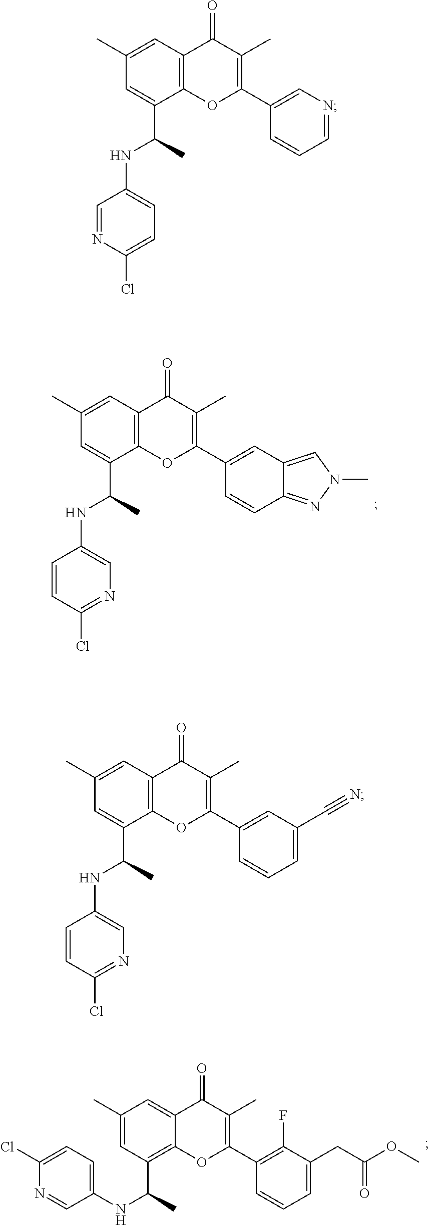 [SMILES: Cc1cc([C@@H](C)Nc2ccc(Cl)nc2)c2oc(-c3cccnc3)c(C)c(=O)c2c1 ; Cc1cc([C@@H](C)Nc2ccc(Cl)nc2)c2oc(-c3ccc4nn(C)cc4c3)c(C)c(=O)c2c1 ; Cc1cc([C@@H](C)Nc2ccc(Cl)nc2)c2oc(-c3cccc(C#N)c3)c(C)c(=O)c2c1 ; COC(=O)Cc1cccc(-c2oc3c([C@@H](C)Nc4ccc(Cl)nc4)cc(C)cc3c(=O)c2C)c1F] 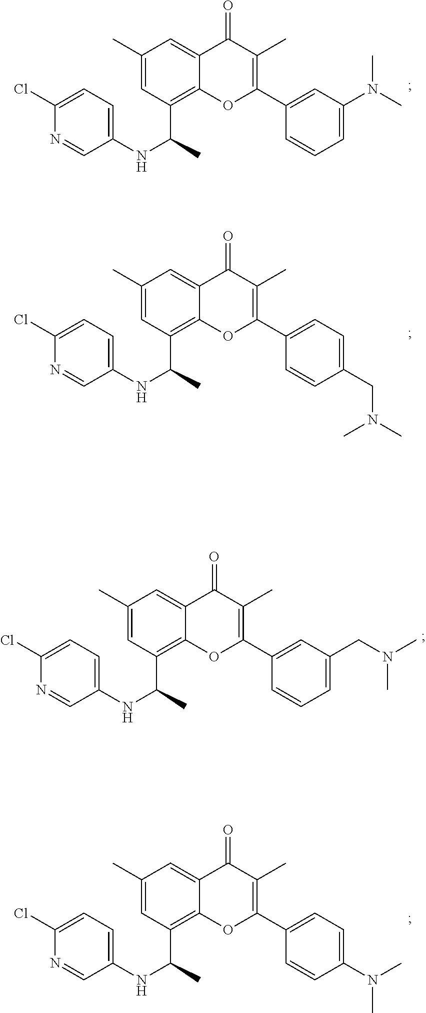 [SMILES: Cc1cc([C@@H](C)Nc2ccc(Cl)nc2)c2oc(-c3cccc(N(C)C)c3)c(C)c(=O)c2c1 ; Cc1cc([C@@H](C)Nc2ccc(Cl)nc2)c2oc(-c3ccc(CN(C)C)cc3)c(C)c(=O)c2c1 ; Cc1cc([C@@H](C)Nc2ccc(Cl)nc2)c2oc(-c3cccc(CN(C)C)c3)c(C)c(=O)c2c1 ; Cc1cc([C@@H](C)Nc2ccc(Cl)nc2)c2oc(-c3ccc(N(C)C)cc3)c(C)c(=O)c2c1] 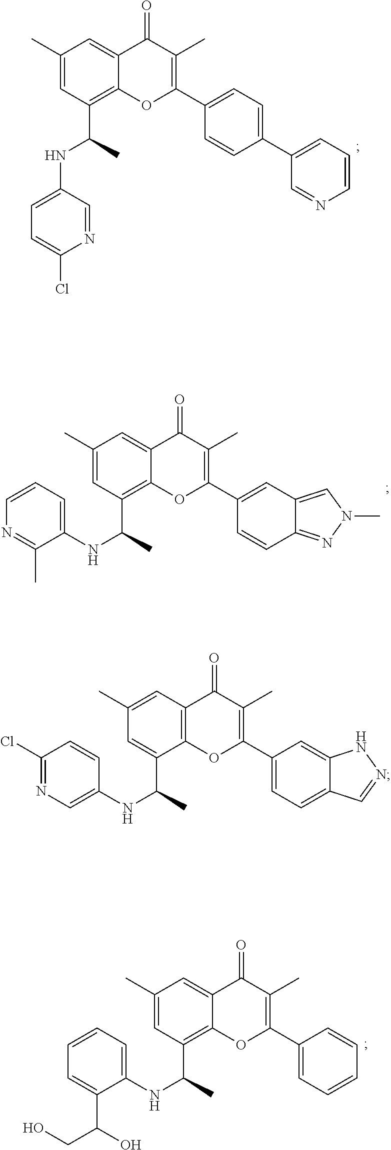 [SMILES: Cc1cc([C@@H](C)Nc2ccc(Cl)nc2)c2oc(-c3ccc(-c4cccnc4)cc3)c(C)c(=O)c2c1 ; Cc1cc([C@@H](C)Nc2cccnc2C)c2oc(-c3ccc4nn(C)cc4c3)c(C)c(=O)c2c1 ; Cc1cc([C@@H](C)Nc2ccc(Cl)nc2)c2oc(-c3ccc4cn[nH]c4c3)c(C)c(=O)c2c1 ; Cc1cc([C@@H](C)Nc2ccccc2C(O)CO)c2oc(-c3ccccc3)c(C)c(=O)c2c1] 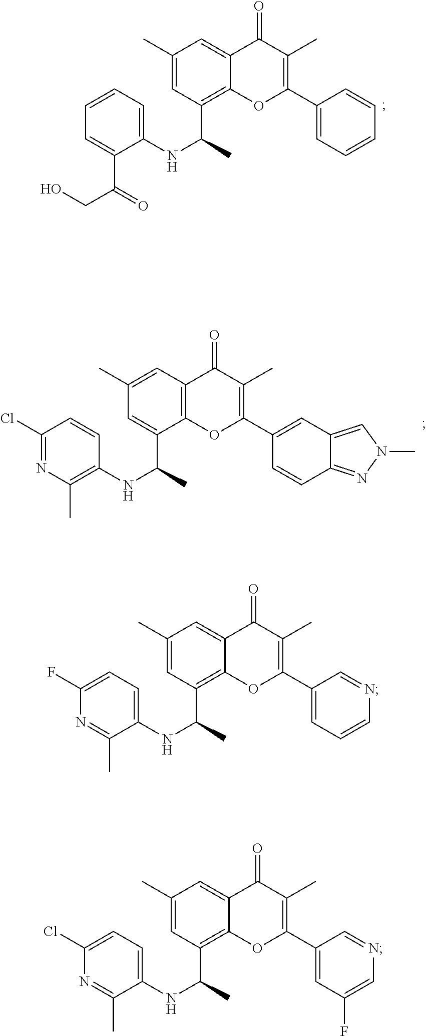 [SMILES: Cc1cc([C@@H](C)Nc2ccccc2C(=O)CO)c2oc(-c3ccccc3)c(C)c(=O)c2c1 ; Cc1cc([C@@H](C)Nc2ccc(Cl)nc2C)c2oc(-c3ccc4nn(C)cc4c3)c(C)c(=O)c2c1 ; Cc1cc([C@@H](C)Nc2ccc(F)nc2C)c2oc(-c3cccnc3)c(C)c(=O)c2c1 ; Cc1cc([C@@H](C)Nc2ccc(Cl)nc2C)c2oc(-c3cncc(F)c3)c(C)c(=O)c2c1] 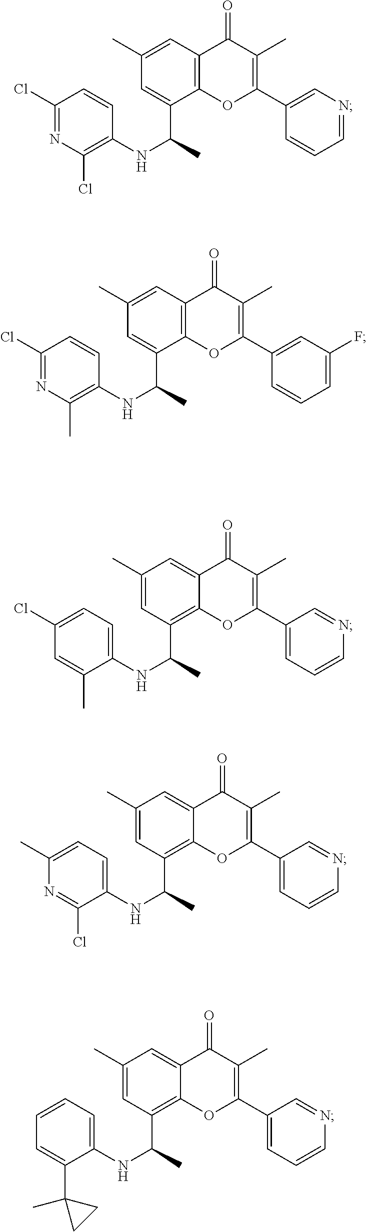 [SMILES: Cc1cc([C@@H](C)Nc2ccc(Cl)nc2Cl)c2oc(-c3cccnc3)c(C)c(=O)c2c1 ; Cc1cc([C@@H](C)Nc2ccc(Cl)nc2C)c2oc(-c3cccc(F)c3)c(C)c(=O)c2c1 ; Cc1cc([C@@H](C)Nc2ccc(Cl)cc2C)c2oc(-c3cccnc3)c(C)c(=O)c2c1 ; Cc1cc([C@@H](C)Nc2ccc(C)nc2Cl)c2oc(-c3cccnc3)c(C)c(=O)c2c1 ; Cc1cc([C@@H](C)Nc2ccccc2C2(C)CC2)c2oc(-c3cccnc3)c(C)c(=O)c2c1] 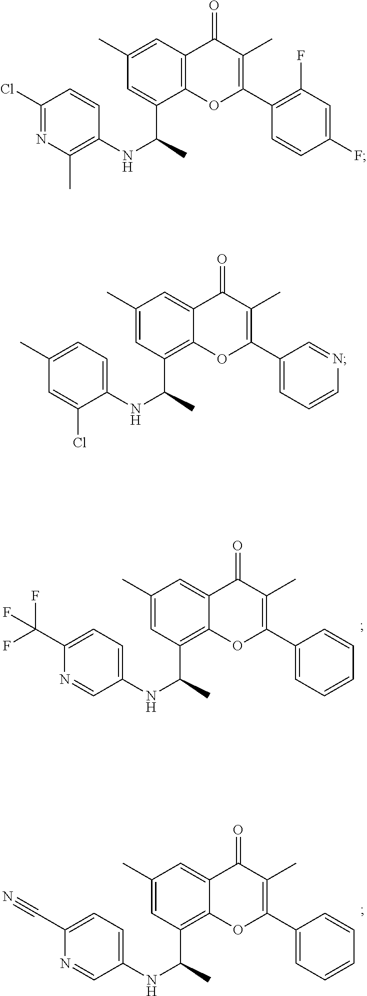 [SMILES: Cc1cc([C@@H](C)Nc2ccc(Cl)nc2C)c2oc(-c3ccc(F)cc3F)c(C)c(=O)c2c1 ; Cc1ccc(N[C@H](C)c2cc(C)cc3c(=O)c(C)c(-c4cccnc4)oc23)c(Cl)c1 ; Cc1cc([C@@H](C)Nc2ccc(C(F)(F)F)nc2)c2oc(-c3ccccc3)c(C)c(=O)c2c1 ; Cc1cc([C@@H](C)Nc2ccc(C#N)nc2)c2oc(-c3ccccc3)c(C)c(=O)c2c1] 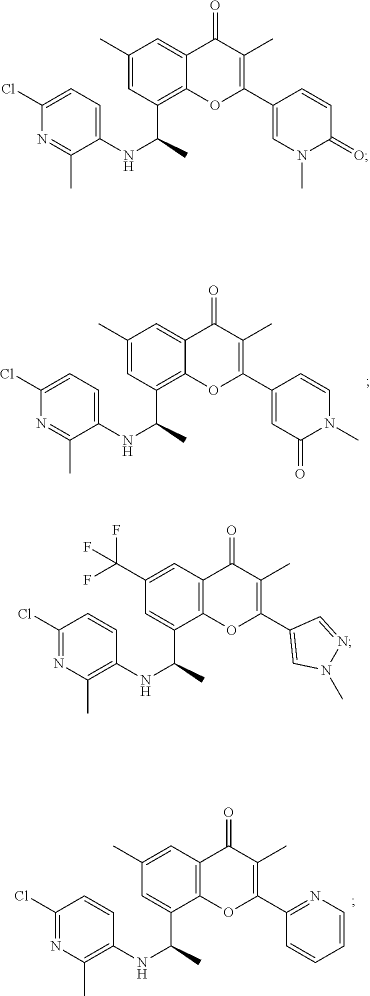 [SMILES: Cc1cc([C@@H](C)Nc2ccc(Cl)nc2C)c2oc(-c3ccc(=O)n(C)c3)c(C)c(=O)c2c1 ; Cc1cc([C@@H](C)Nc2ccc(Cl)nc2C)c2oc(-c3ccn(C)c(=O)c3)c(C)c(=O)c2c1 ; Cc1nc(Cl)ccc1N[C@H](C)c1cc(C(F)(F)F)cc2c(=O)c(C)c(-c3cnn(C)c3)oc12 ; Cc1cc([C@@H](C)Nc2ccc(Cl)nc2C)c2oc(-c3ccccn3)c(C)c(=O)c2c1] 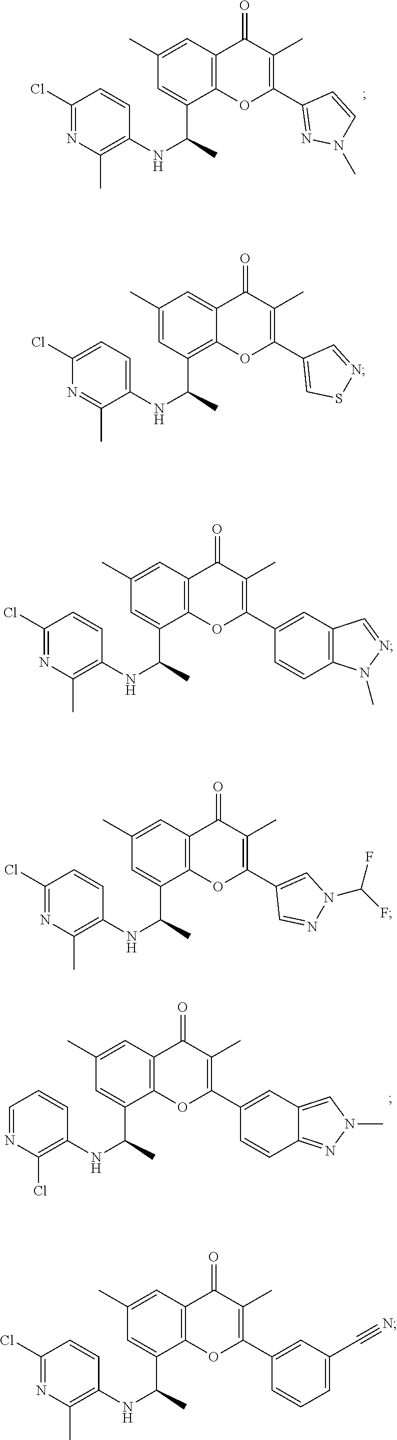 [SMILES: Cc1cc([C@@H](C)Nc2ccc(Cl)nc2C)c2oc(-c3ccn(C)n3)c(C)c(=O)c2c1 ; Cc1cc([C@@H](C)Nc2ccc(Cl)nc2C)c2oc(-c3cnsc3)c(C)c(=O)c2c1 ; Cc1cc([C@@H](C)Nc2ccc(Cl)nc2C)c2oc(-c3ccc4c(cnn4C)c3)c(C)c(=O)c2c1 ; Cc1cc([C@@H](C)Nc2ccc(Cl)nc2C)c2oc(-c3cnn(C(F)F)c3)c(C)c(=O)c2c1 ; Cc1cc([C@@H](C)Nc2cccnc2Cl)c2oc(-c3ccc4nn(C)cc4c3)c(C)c(=O)c2c1 ; Cc1cc([C@@H](C)Nc2ccc(Cl)nc2C)c2oc(-c3cccc(C#N)c3)c(C)c(=O)c2c1] 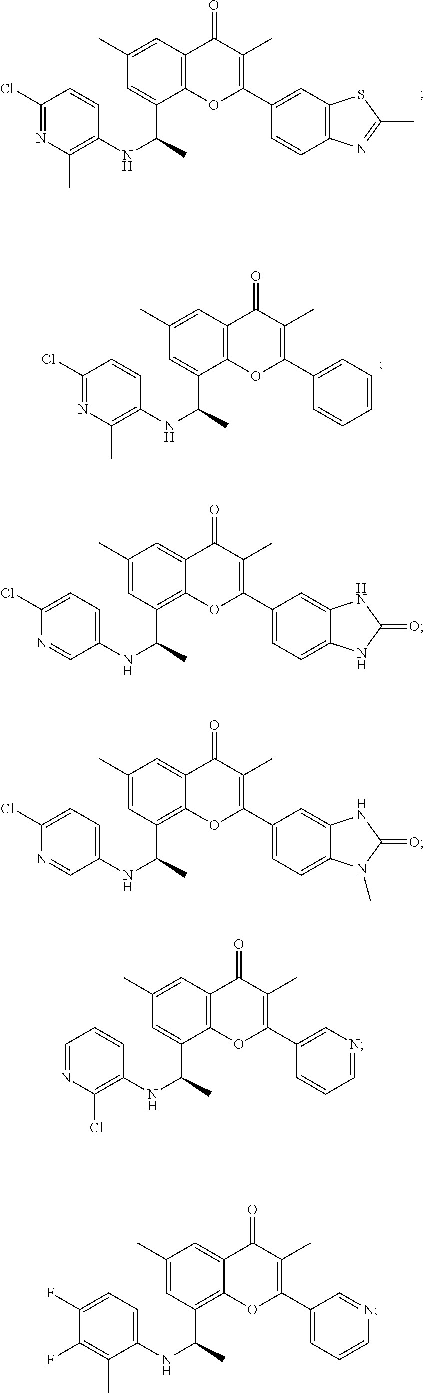 [SMILES: Cc1cc([C@@H](C)Nc2ccc(Cl)nc2C)c2oc(-c3ccc4nc(C)sc4c3)c(C)c(=O)c2c1 ; Cc1cc([C@@H](C)Nc2ccc(Cl)nc2C)c2oc(-c3ccccc3)c(C)c(=O)c2c1 ; Cc1cc([C@@H](C)Nc2ccc(Cl)nc2)c2oc(-c3ccc4[nH]c(=O)[nH]c4c3)c(C)c(=O)c2c1 ; Cc1cc([C@@H](C)Nc2ccc(Cl)nc2)c2oc(-c3ccc4c(c3)[nH]c(=O)n4C)c(C)c(=O)c2c1 ; Cc1cc([C@@H](C)Nc2cccnc2Cl)c2oc(-c3cccnc3)c(C)c(=O)c2c1 ; Cc1cc([C@@H](C)Nc2ccc(F)c(F)c2C)c2oc(-c3cccnc3)c(C)c(=O)c2c1] 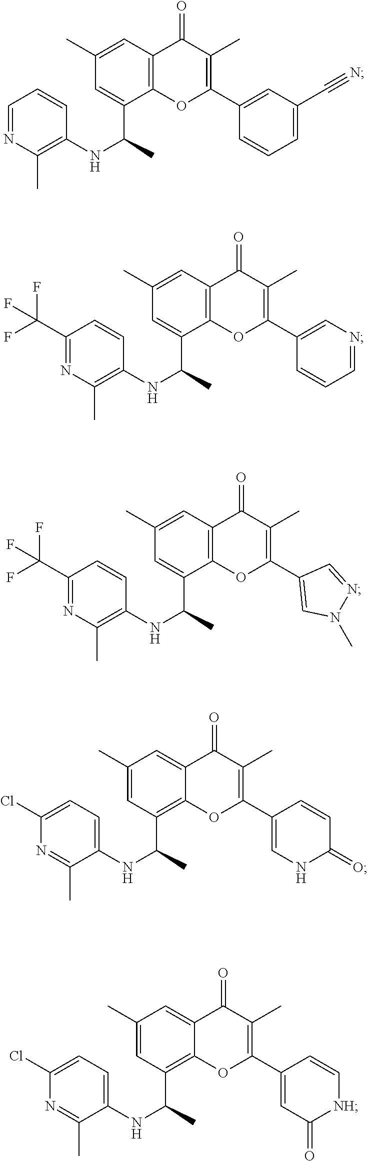 [SMILES: Cc1cc([C@@H](C)Nc2cccnc2C)c2oc(-c3cccc(C#N)c3)c(C)c(=O)c2c1 ; Cc1cc([C@@H](C)Nc2ccc(C(F)(F)F)nc2C)c2oc(-c3cccnc3)c(C)c(=O)c2c1 ; Cc1cc([C@@H](C)Nc2ccc(C(F)(F)F)nc2C)c2oc(-c3cnn(C)c3)c(C)c(=O)c2c1 ; Cc1cc([C@@H](C)Nc2ccc(Cl)nc2C)c2oc(-c3ccc(=O)[nH]c3)c(C)c(=O)c2c1 ; Cc1cc([C@@H](C)Nc2ccc(Cl)nc2C)c2oc(-c3cc[nH]c(=O)c3)c(C)c(=O)c2c1] 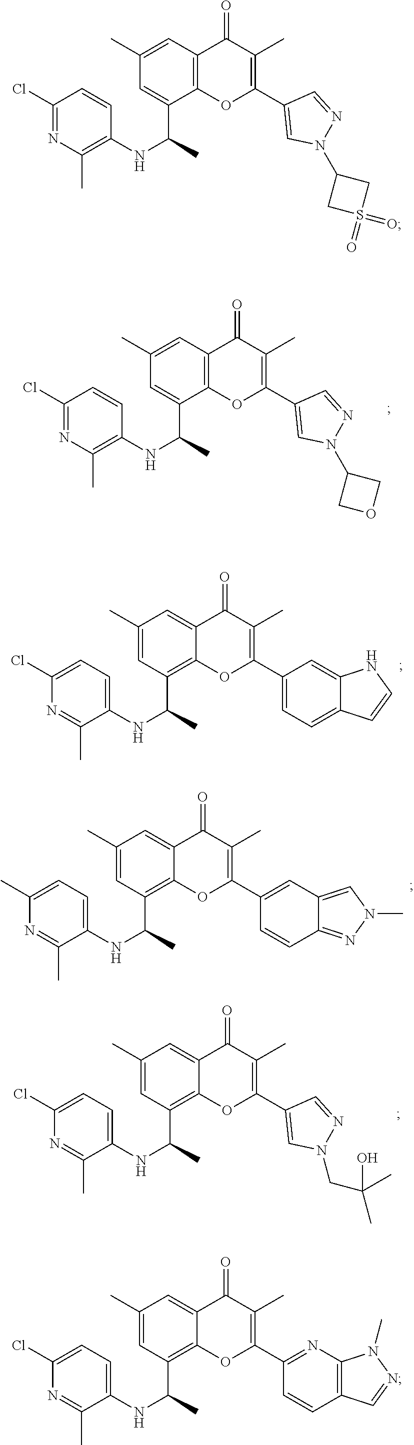 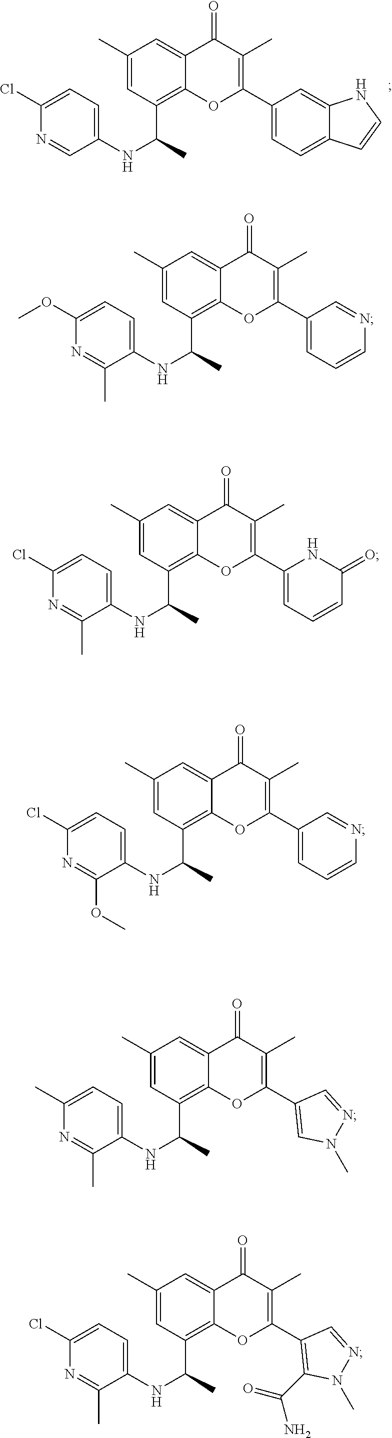 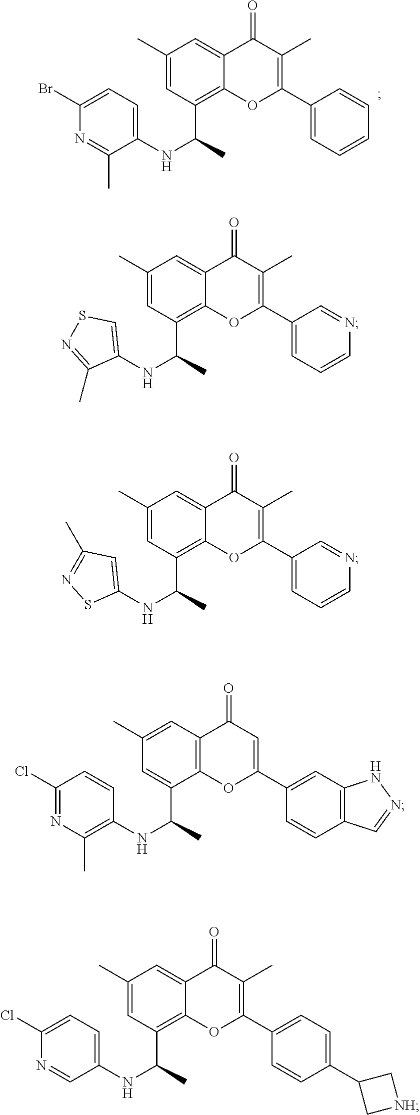 [SMILES: Cc1cc([C@@H](C)Nc2ccc(Br)nc2C)c2oc(-c3ccccc3)c(C)c(=O)c2c1 ; Cc1cc([C@@H](C)Nc2csnc2C)c2oc(-c3cccnc3)c(C)c(=O)c2c1 ; Cc1cc([C@@H](C)Nc2cc(C)ns2)c2oc(-c3cccnc3)c(C)c(=O)c2c1 ; Cc1cc([C@@H](C)Nc2ccc(Cl)nc2C)c2oc(-c3ccc4cn[nH]c4c3)cc(=O)c2c1 ; Cc1cc([C@@H](C)Nc2ccc(Cl)nc2)c2oc(-c3ccc(C4CNC4)cc3)c(C)c(=O)c2c1] 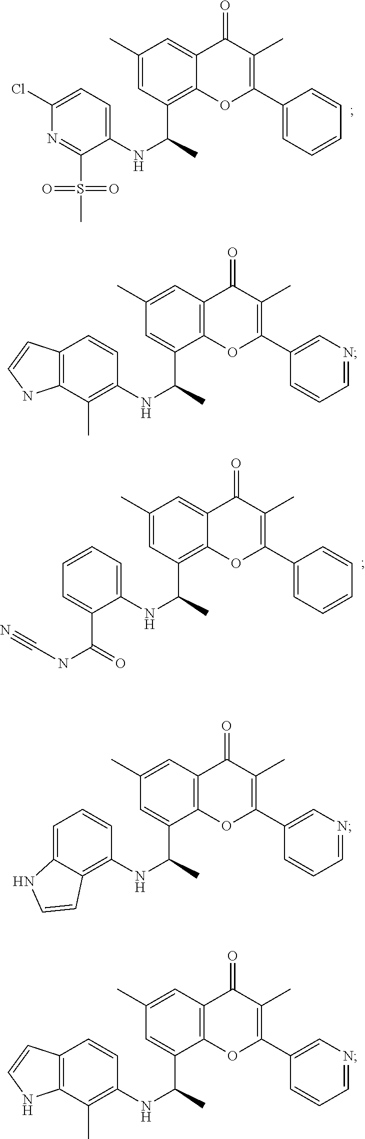 [SMILES: Cc1cc([C@@H](C)Nc2ccc(Cl)nc2S(C)(=O)=O)c2oc(-c3ccccc3)c(C)c(=O)c2c1 ; Cc1cc([C@@H](C)Nc2ccc3cc[nH]c3c2C)c2oc(-c3cccnc3)c(C)c(=O)c2c1 ; Cc1cc([C@@H](C)Nc2ccccc2C(=O)NC#N)c2oc(-c3ccccc3)c(C)c(=O)c2c1 ; Cc1cc([C@@H](C)Nc2cccc3[nH]ccc23)c2oc(-c3cccnc3)c(C)c(=O)c2c1 ; Cc1cc([C@@H](C)Nc2ccc3cc[nH]c3c2C)c2oc(-c3cccnc3)c(C)c(=O)c2c1] 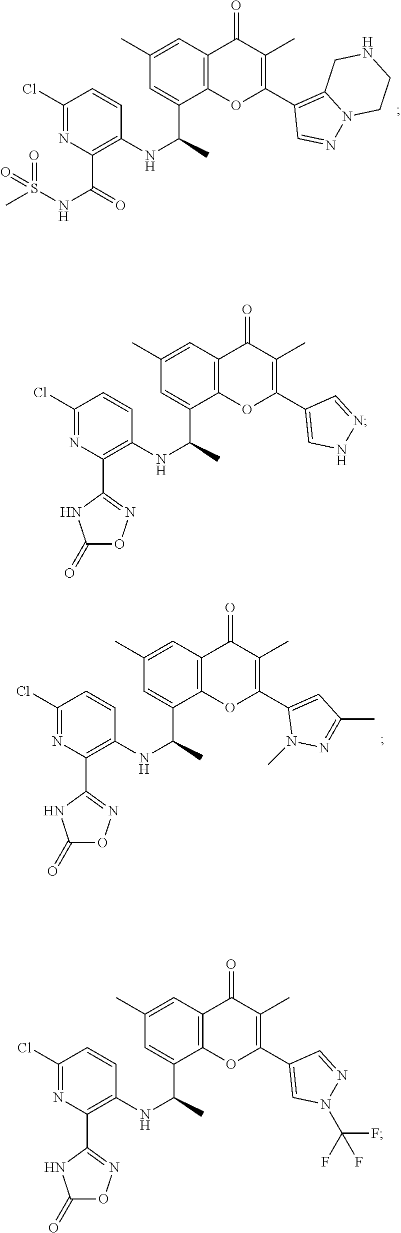 [SMILES: Cc1cc([C@@H](C)Nc2ccc(Cl)nc2C(=O)NS(C)(=O)=O)c2oc(-c3cnn4c3CNCC4)c(C)c(=O)c2c1 ; Cc1cc([C@@H](C)Nc2ccc(Cl)nc2-c2noc(=O)[nH]2)c2oc(-c3cn[nH]c3)c(C)c(=O)c2c1 ; Cc1cc([C@@H](C)Nc2ccc(Cl)nc2-c2noc(=O)[nH]2)c2oc(-c3cc(C)nn3C)c(C)c(=O)c2c1 ; Cc1cc([C@@H](C)Nc2ccc(Cl)nc2-c2noc(=O)[nH]2)c2oc(-c3cnn(C(F)(F)F)c3)c(C)c(=O)c2c1] 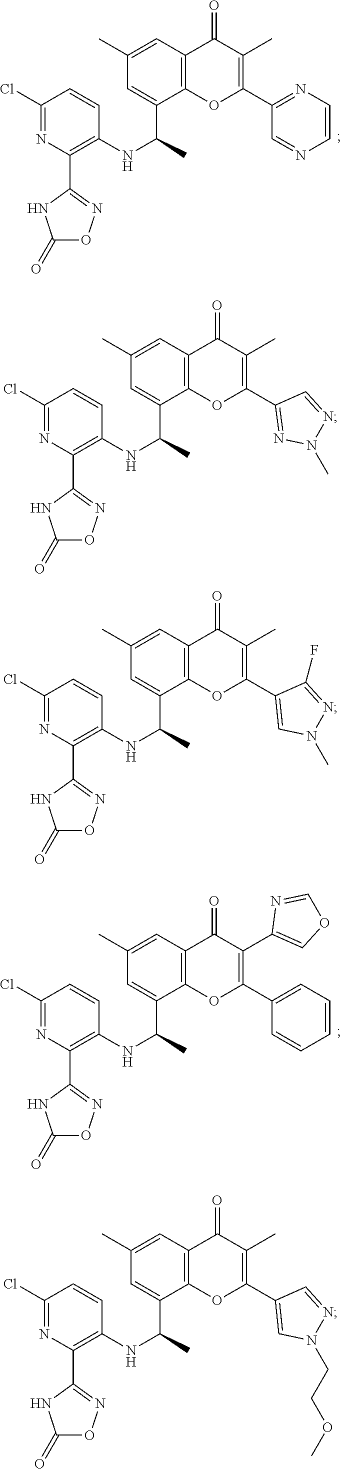 [SMILES: Cc1cc([C@@H](C)Nc2ccc(Cl)nc2-c2noc(=O)[nH]2)c2oc(-c3cnccn3)c(C)c(=O)c2c1 ; Cc1cc([C@@H](C)Nc2ccc(Cl)nc2-c2noc(=O)[nH]2)c2oc(-c3cnn(C)n3)c(C)c(=O)c2c1 ; Cc1cc([C@@H](C)Nc2ccc(Cl)nc2-c2noc(=O)[nH]2)c2oc(-c3cn(C)nc3F)c(C)c(=O)c2c1 ; Cc1cc([C@@H](C)Nc2ccc(Cl)nc2-c2noc(=O)[nH]2)c2oc(-c3ccccc3)c(-c3cocn3)c(=O)c2c1 ; COCCn1cc(-c2oc3c([C@@H](C)Nc4ccc(Cl)nc4-c4noc(=O)[nH]4)cc(C)cc3c(=O)c2C)cn1] 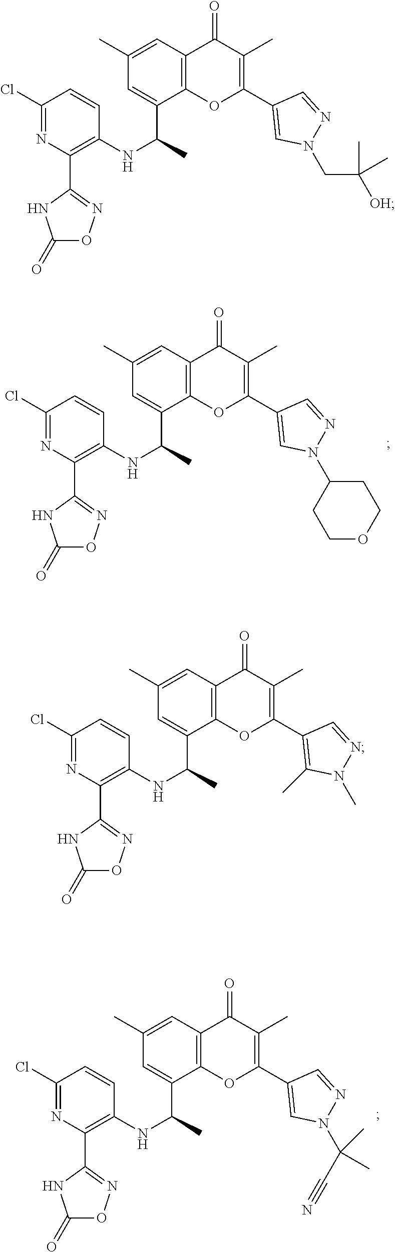 [SMILES: Cc1cc([C@@H](C)Nc2ccc(Cl)nc2-c2noc(=O)[nH]2)c2oc(-c3cnn(CC(C)(C)O)c3)c(C)c(=O)c2c1 ; Cc1cc([C@@H](C)Nc2ccc(Cl)nc2-c2noc(=O)[nH]2)c2oc(-c3cnn(C4CCOCC4)c3)c(C)c(=O)c2c1 ; Cc1cc([C@@H](C)Nc2ccc(Cl)nc2-c2noc(=O)[nH]2)c2oc(-c3cnn(C)c3C)c(C)c(=O)c2c1 ; Cc1cc([C@@H](C)Nc2ccc(Cl)nc2-c2noc(=O)[nH]2)c2oc(-c3cnn(C(C)(C)C#N)c3)c(C)c(=O)c2c1] 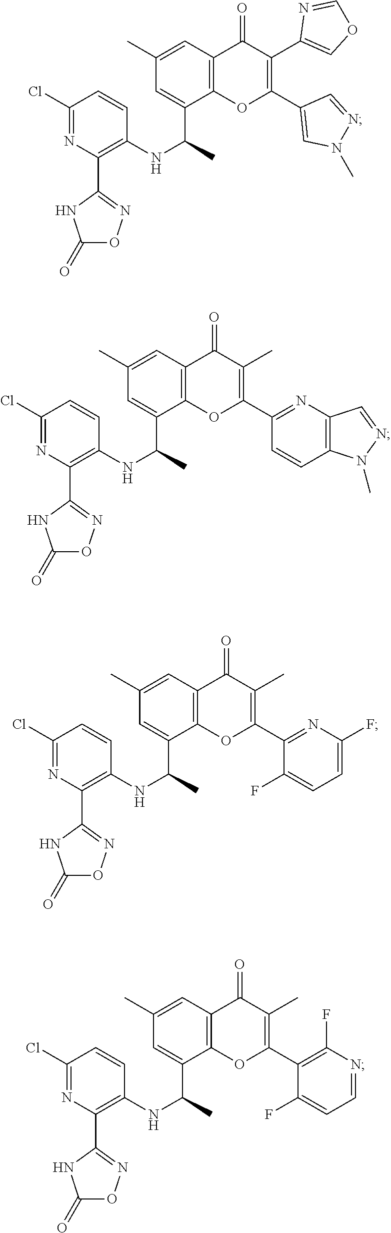 [SMILES: Cc1cc([C@@H](C)Nc2ccc(Cl)nc2-c2noc(=O)[nH]2)c2oc(-c3cnn(C)c3)c(-c3cocn3)c(=O)c2c1 ; Cc1cc([C@@H](C)Nc2ccc(Cl)nc2-c2noc(=O)[nH]2)c2oc(-c3ccc4c(cnn4C)n3)c(C)c(=O)c2c1 ; Cc1cc([C@@H](C)Nc2ccc(Cl)nc2-c2noc(=O)[nH]2)c2oc(-c3nc(F)ccc3F)c(C)c(=O)c2c1 ; Cc1cc([C@@H](C)Nc2ccc(Cl)nc2-c2noc(=O)[nH]2)c2oc(-c3c(F)ccnc3F)c(C)c(=O)c2c1] 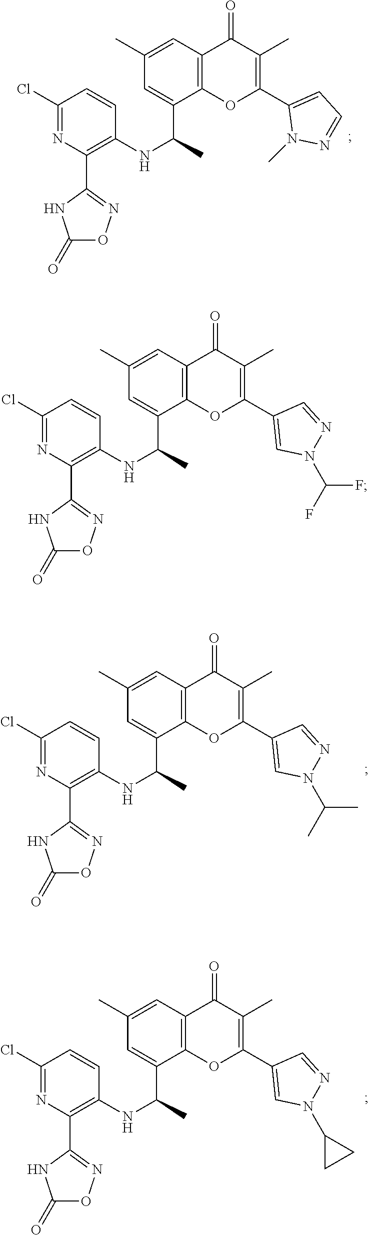 [SMILES: Cc1cc([C@@H](C)Nc2ccc(Cl)nc2-c2noc(=O)[nH]2)c2oc(-c3ccnn3C)c(C)c(=O)c2c1 ; Cc1cc([C@@H](C)Nc2ccc(Cl)nc2-c2noc(=O)[nH]2)c2oc(-c3cnn(C(F)F)c3)c(C)c(=O)c2c1 ; Cc1cc([C@@H](C)Nc2ccc(Cl)nc2-c2noc(=O)[nH]2)c2oc(-c3cnn(C(C)C)c3)c(C)c(=O)c2c1 ; Cc1cc([C@@H](C)Nc2ccc(Cl)nc2-c2noc(=O)[nH]2)c2oc(-c3cnn(C4CC4)c3)c(C)c(=O)c2c1] 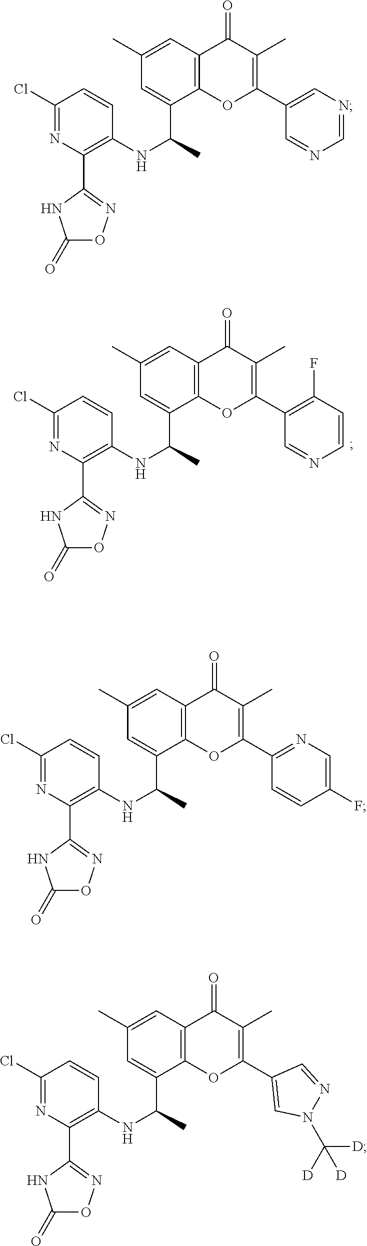 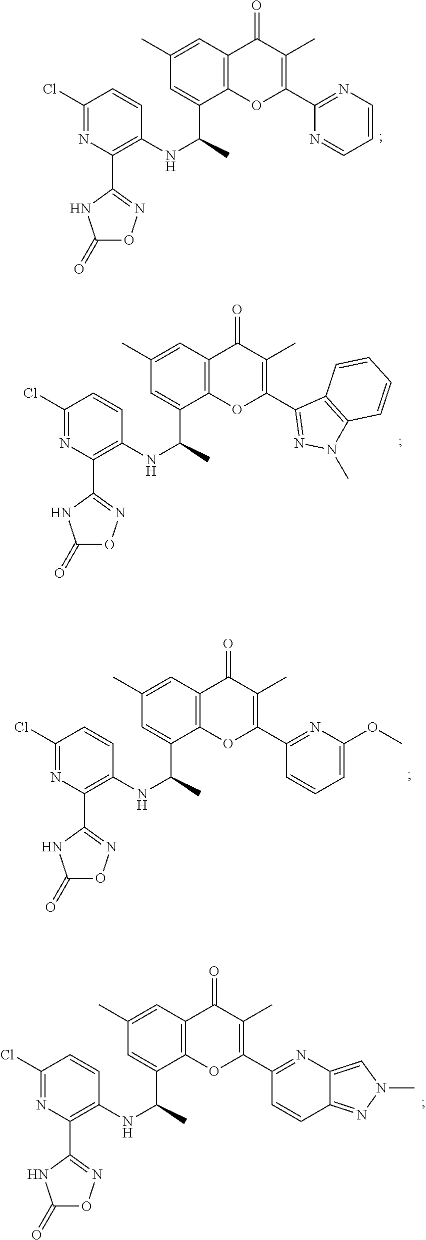 [SMILES: Cc1cc([C@@H](C)Nc2ccc(Cl)nc2-c2noc(=O)[nH]2)c2oc(-c3ncccn3)c(C)c(=O)c2c1 ; Cc1cc([C@@H](C)Nc2ccc(Cl)nc2-c2noc(=O)[nH]2)c2oc(-c3nn(C)c4ccccc34)c(C)c(=O)c2c1 ; COc1cccc(-c2oc3c([C@@H](C)Nc4ccc(Cl)nc4-c4noc(=O)[nH]4)cc(C)cc3c(=O)c2C)n1 ; Cc1cc([C@@H](C)Nc2ccc(Cl)nc2-c2noc(=O)[nH]2)c2oc(-c3ccc4nn(C)cc4n3)c(C)c(=O)c2c1] 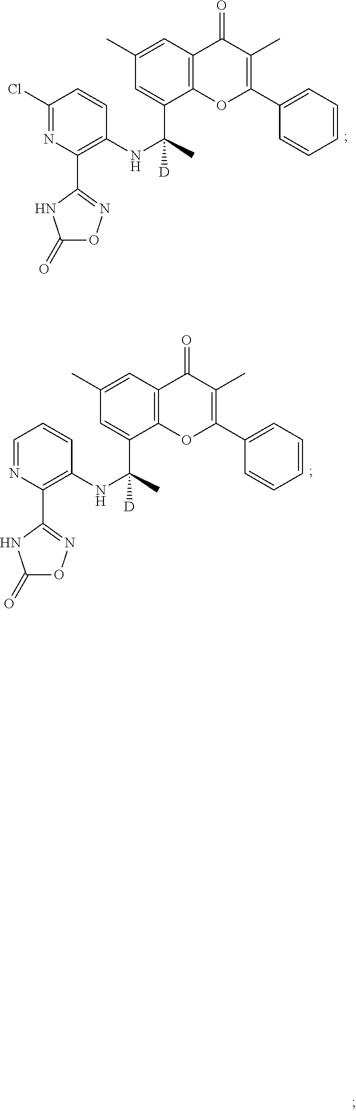 [SMILES: [2H][C@](C)(Nc1ccc(Cl)nc1-c1noc(=O)[nH]1)c1cc(C)cc2c(=O)c(C)c(-c3ccccc3)oc12 ; [2H][C@](C)(Nc1cccnc1-c1noc(=O)[nH]1)c1cc(C)cc2c(=O)c(C)c(-c3ccccc3)oc12] 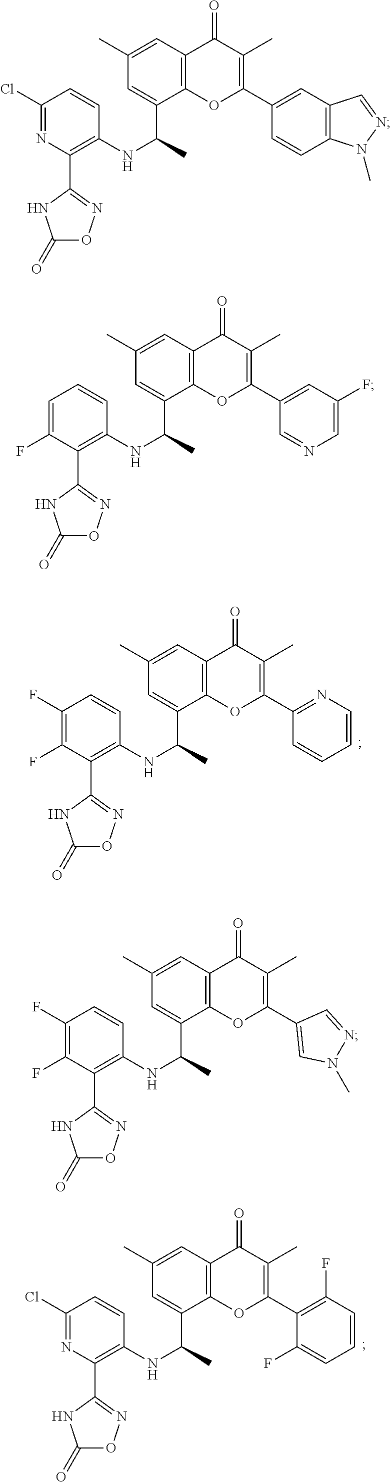 [SMILES: Cc1cc([C@@H](C)Nc2ccc(Cl)nc2-c2noc(=O)[nH]2)c2oc(-c3ccc4c(cnn4C)c3)c(C)c(=O)c2c1 ; Cc1cc([C@@H](C)Nc2cccc(F)c2-c2noc(=O)[nH]2)c2oc(-c3cncc(F)c3)c(C)c(=O)c2c1 ; Cc1cc([C@@H](C)Nc2ccc(F)c(F)c2-c2noc(=O)[nH]2)c2oc(-c3ccccn3)c(C)c(=O)c2c1 ; Cc1cc([C@@H](C)Nc2ccc(F)c(F)c2-c2noc(=O)[nH]2)c2oc(-c3cnn(C)c3)c(C)c(=O)c2c1 ; Cc1cc([C@@H](C)Nc2ccc(Cl)nc2-c2noc(=O)[nH]2)c2oc(-c3c(F)cccc3F)c(C)c(=O)c2c1] 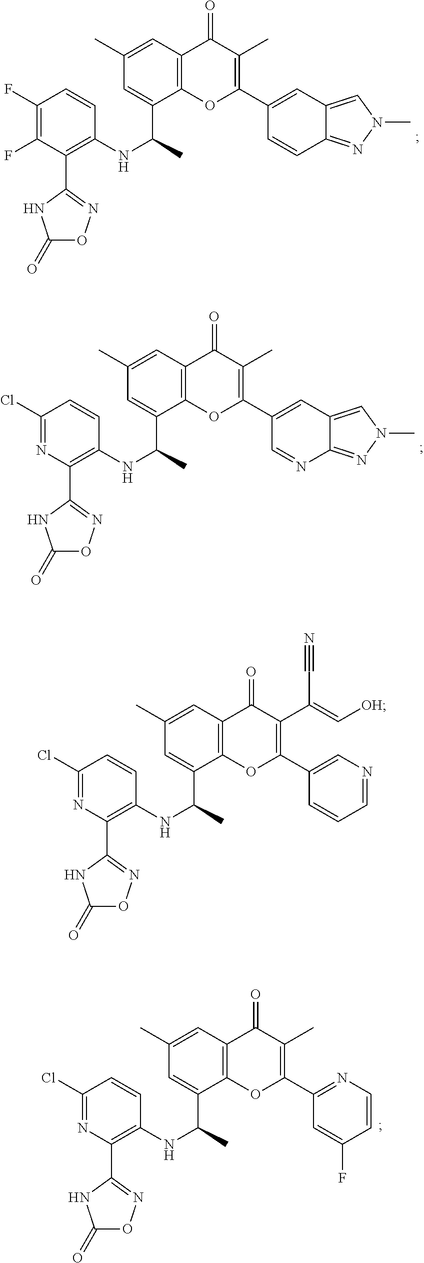 [SMILES: Cc1cc([C@@H](C)Nc2ccc(F)c(F)c2-c2noc(=O)[nH]2)c2oc(-c3ccc4nn(C)cc4c3)c(C)c(=O)c2c1 ; Cc1cc([C@@H](C)Nc2ccc(Cl)nc2-c2noc(=O)[nH]2)c2oc(-c3cnc4nn(C)cc4c3)c(C)c(=O)c2c1 ; Cc1cc([C@@H](C)Nc2ccc(Cl)nc2-c2noc(=O)[nH]2)c2oc(-c3cccnc3)c(/C(C#N)=C/O)c(=O)c2c1 ; Cc1cc([C@@H](C)Nc2ccc(Cl)nc2-c2noc(=O)[nH]2)c2oc(-c3cc(F)ccn3)c(C)c(=O)c2c1] 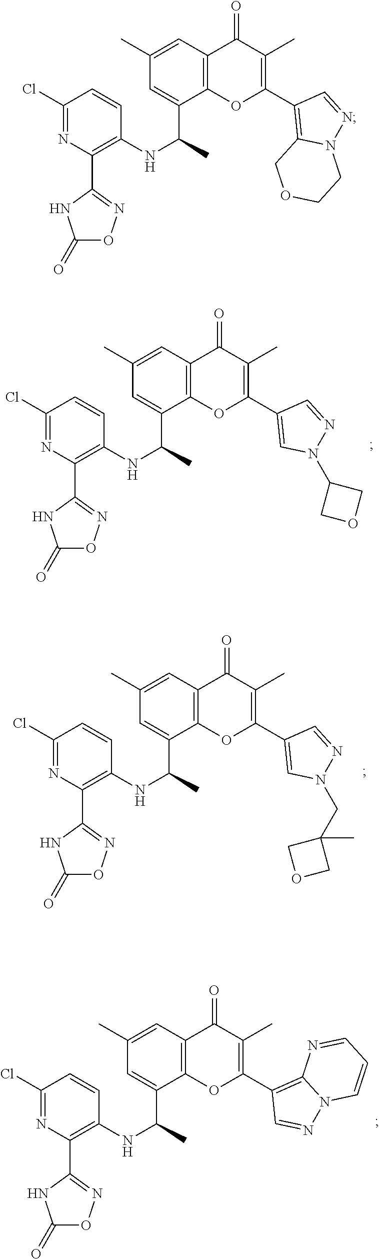 [SMILES: Cc1cc([C@@H](C)Nc2ccc(Cl)nc2-c2noc(=O)[nH]2)c2oc(-c3cnn4c3COCC4)c(C)c(=O)c2c1 ; Cc1cc([C@@H](C)Nc2ccc(Cl)nc2-c2noc(=O)[nH]2)c2oc(-c3cnn(C4COC4)c3)c(C)c(=O)c2c1 ; Cc1cc([C@@H](C)Nc2ccc(Cl)nc2-c2noc(=O)[nH]2)c2oc(-c3cnn(CC4(C)COC4)c3)c(C)c(=O)c2c1 ; Cc1cc([C@@H](C)Nc2ccc(Cl)nc2-c2noc(=O)[nH]2)c2oc(-c3cnn4cccnc34)c(C)c(=O)c2c1] 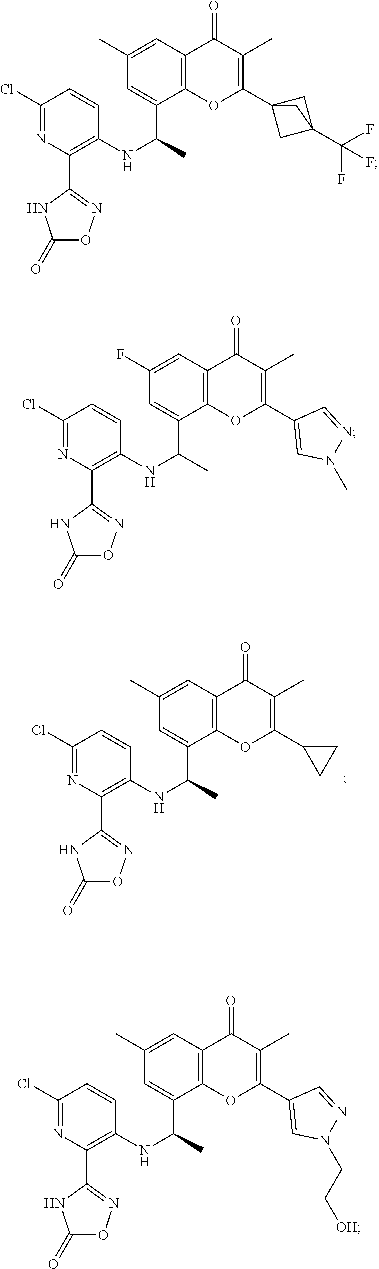 [SMILES: Cc1cc([C@@H](C)Nc2ccc(Cl)nc2-c2noc(=O)[nH]2)c2oc(C34CC(C(F)(F)F)(C3)C4)c(C)c(=O)c2c1 ; Cc1c(-c2cnn(C)c2)oc2c(C(C)Nc3ccc(Cl)nc3-c3noc(=O)[nH]3)cc(F)cc2c1=O ; Cc1cc([C@@H](C)Nc2ccc(Cl)nc2-c2noc(=O)[nH]2)c2oc(C3CC3)c(C)c(=O)c2c1 ; Cc1cc([C@@H](C)Nc2ccc(Cl)nc2-c2noc(=O)[nH]2)c2oc(-c3cnn(CCO)c3)c(C)c(=O)c2c1] 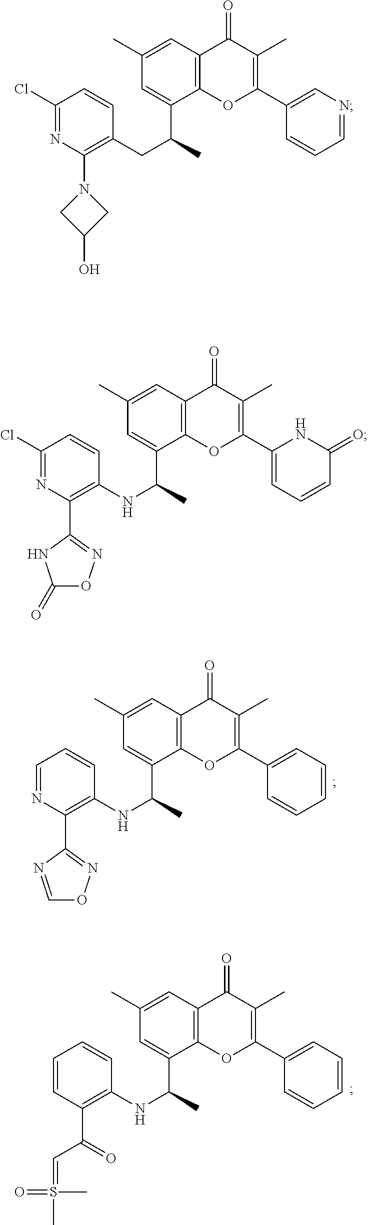 [SMILES: Cc1cc([C@@H](C)Cc2ccc(Cl)nc2N2CC(O)C2)c2oc(-c3cccnc3)c(C)c(=O)c2c1 ; Cc1cc([C@@H](C)Nc2ccc(Cl)nc2-c2noc(=O)[nH]2)c2oc(-c3cccc(=O)[nH]3)c(C)c(=O)c2c1 ; Cc1cc([C@@H](C)Nc2cccnc2-c2ncon2)c2oc(-c3ccccc3)c(C)c(=O)c2c1 ; Cc1cc([C@@H](C)Nc2ccccc2C(=O)C=S(C)(C)=O)c2oc(-c3ccccc3)c(C)c(=O)c2c1] 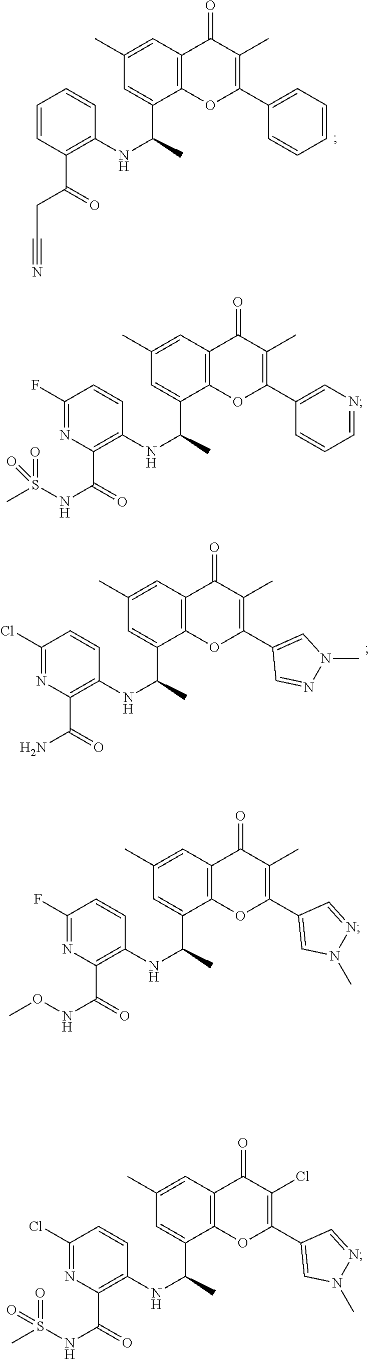 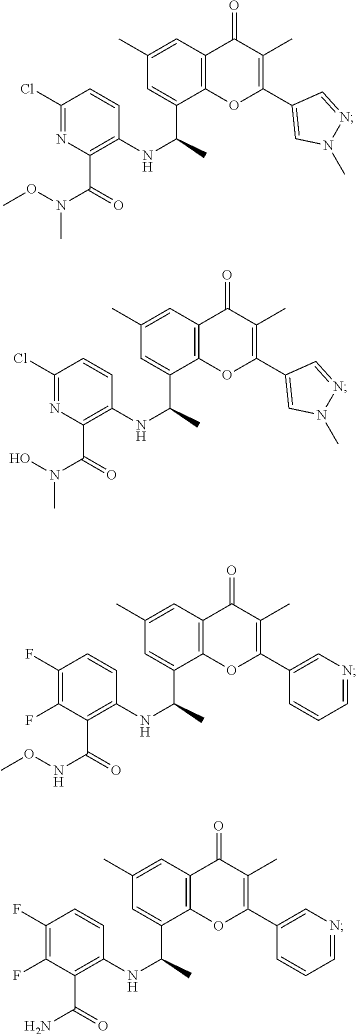 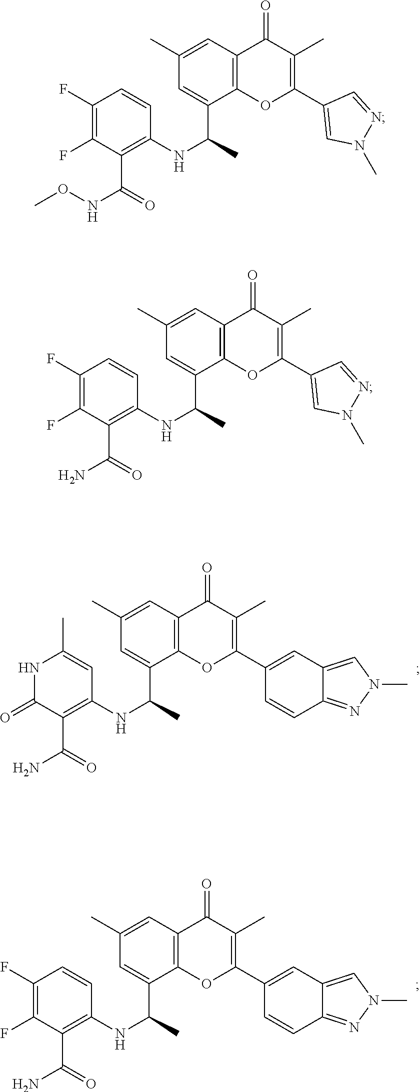 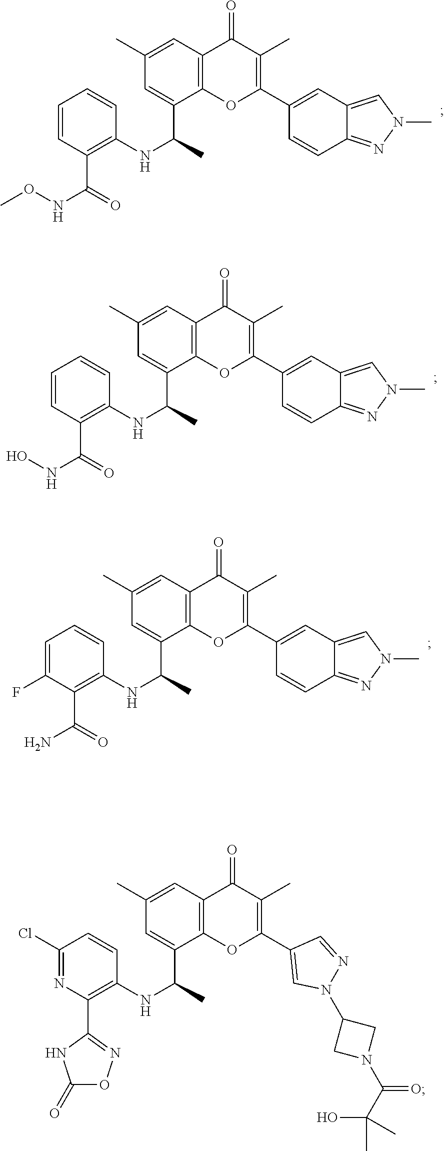 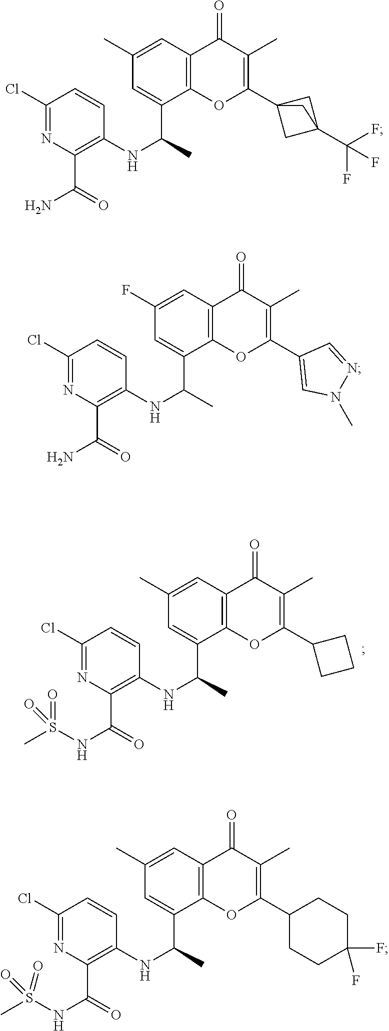 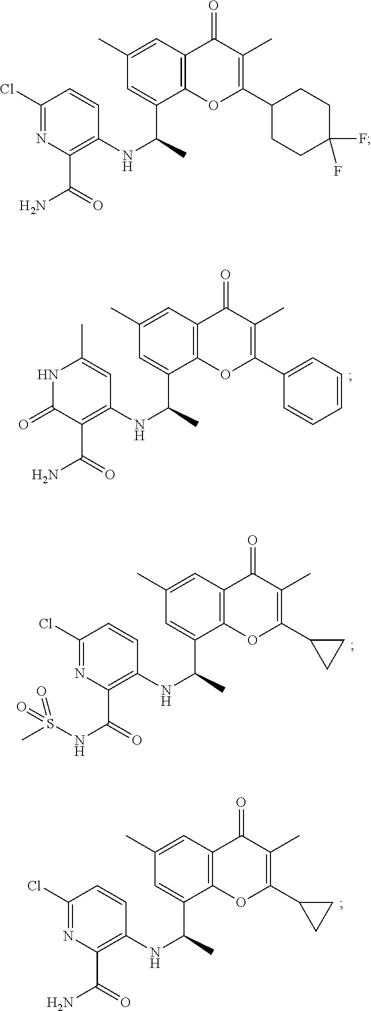 [SMILES: Cc1cc([C@@H](C)Nc2ccc(Cl)nc2C(N)=O)c2oc(C3CCC(F)(F)CC3)c(C)c(=O)c2c1 ; Cc1cc([C@@H](C)Nc2cc(C)[nH]c(=O)c2C(N)=O)c2oc(-c3ccccc3)c(C)c(=O)c2c1 ; Cc1cc([C@@H](C)Nc2ccc(Cl)nc2C(=O)NS(C)(=O)=O)c2oc(C3CC3)c(C)c(=O)c2c1 ; Cc1cc([C@@H](C)Nc2ccc(Cl)nc2C(N)=O)c2oc(C3CC3)c(C)c(=O)c2c1] 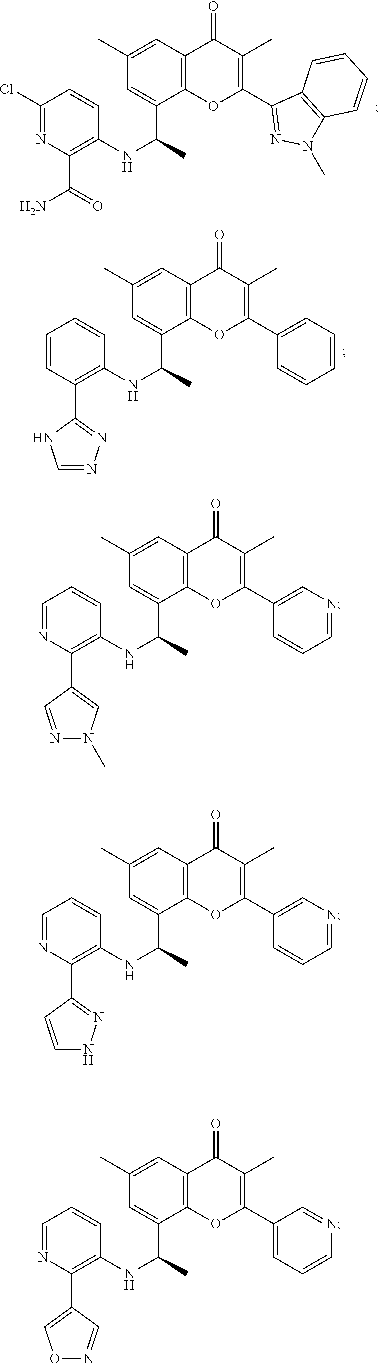 [SMILES: Cc1cc([C@@H](C)Nc2ccc(Cl)nc2C(N)=O)c2oc(-c3nn(C)c4ccccc34)c(C)c(=O)c2c1 ; Cc1cc([C@@H](C)Nc2ccccc2-c2nnc[nH]2)c2oc(-c3ccccc3)c(C)c(=O)c2c1 ; Cc1cc([C@@H](C)Nc2cccnc2-c2cnn(C)c2)c2oc(-c3cccnc3)c(C)c(=O)c2c1 ; Cc1cc([C@@H](C)Nc2cccnc2-c2cc[nH]n2)c2oc(-c3cccnc3)c(C)c(=O)c2c1 ; Cc1cc([C@@H](C)Nc2cccnc2-c2cnoc2)c2oc(-c3cccnc3)c(C)c(=O)c2c1] 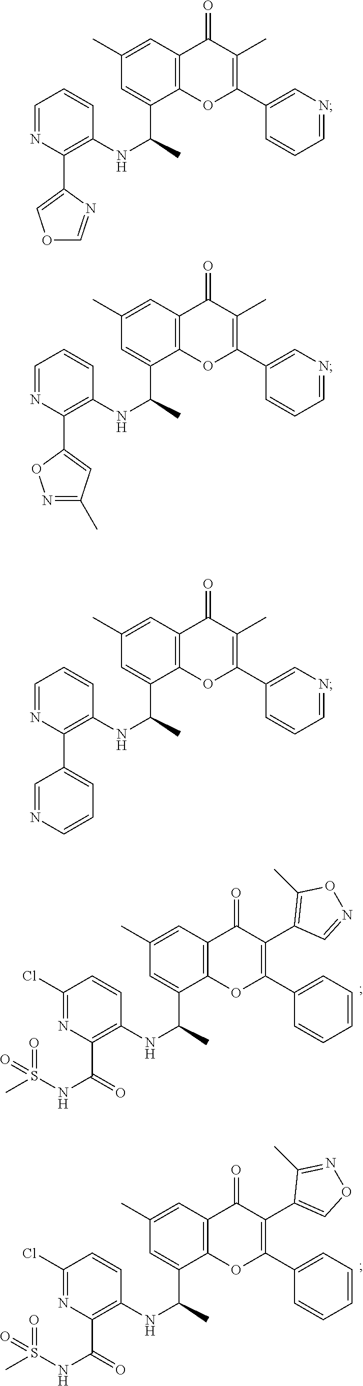 [SMILES: Cc1cc([C@@H](C)Nc2cccnc2-c2cocn2)c2oc(-c3cccnc3)c(C)c(=O)c2c1 ; Cc1cc([C@@H](C)Nc2cccnc2-c2cc(C)no2)c2oc(-c3cccnc3)c(C)c(=O)c2c1 ; Cc1cc([C@@H](C)Nc2cccnc2-c2cccnc2)c2oc(-c3cccnc3)c(C)c(=O)c2c1 ; Cc1cc([C@@H](C)Nc2ccc(Cl)nc2C(=O)NS(C)(=O)=O)c2oc(-c3ccccc3)c(-c3cnoc3C)c(=O)c2c1 ; Cc1cc([C@@H](C)Nc2ccc(Cl)nc2C(=O)NS(C)(=O)=O)c2oc(-c3ccccc3)c(-c3conc3C)c(=O)c2c1] 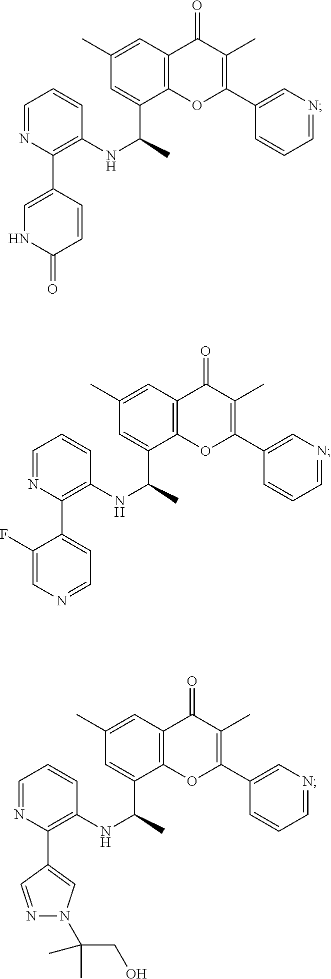 [SMILES: Cc1cc([C@@H](C)Nc2cccnc2-c2ccc(=O)[nH]c2)c2oc(-c3cccnc3)c(C)c(=O)c2c1 ; Cc1cc([C@@H](C)Nc2cccnc2-c2ccncc2F)c2oc(-c3cccnc3)c(C)c(=O)c2c1 ; Cc1cc([C@@H](C)Nc2cccnc2-c2cnn(C(C)(C)CO)c2)c2oc(-c3cccnc3)c(C)c(=O)c2c1] 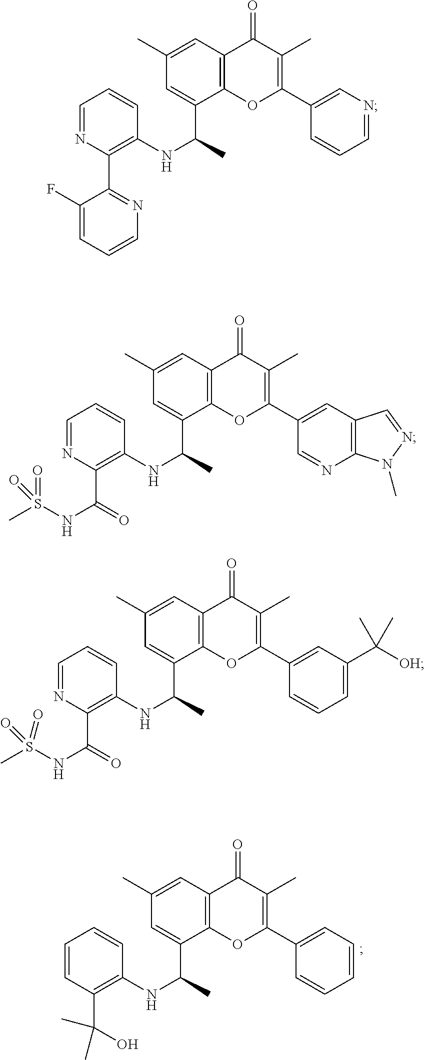 [SMILES: Cc1cc([C@@H](C)Nc2cccnc2-c2ncccc2F)c2oc(-c3cccnc3)c(C)c(=O)c2c1 ; Cc1cc([C@@H](C)Nc2cccnc2C(=O)NS(C)(=O)=O)c2oc(-c3cnc4c(cnn4C)c3)c(C)c(=O)c2c1 ; Cc1cc([C@@H](C)Nc2cccnc2C(=O)NS(C)(=O)=O)c2oc(-c3cccc(C(C)(C)O)c3)c(C)c(=O)c2c1 ; Cc1cc([C@@H](C)Nc2ccccc2C(C)(C)O)c2oc(-c3ccccc3)c(C)c(=O)c2c1] 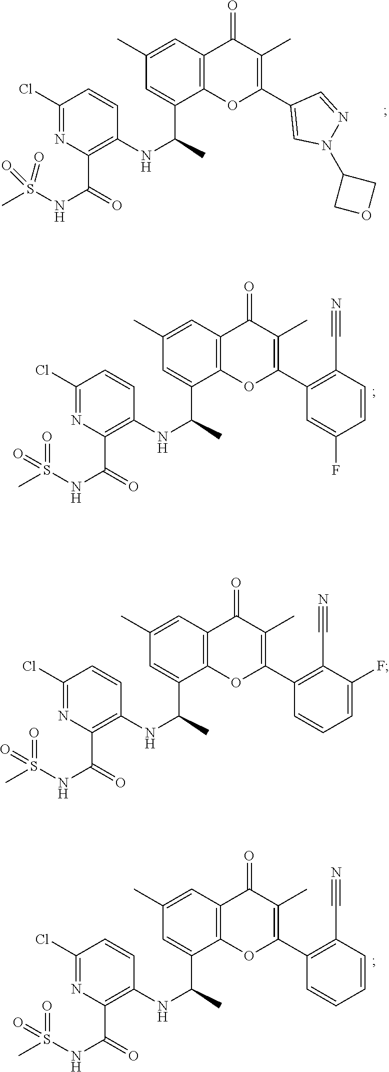 [SMILES: Cc1cc([C@@H](C)Nc2ccc(Cl)nc2C(=O)NS(C)(=O)=O)c2oc(-c3cnn(C4COC4)c3)c(C)c(=O)c2c1 ; Cc1cc([C@@H](C)Nc2ccc(Cl)nc2C(=O)NS(C)(=O)=O)c2oc(-c3cc(F)ccc3C#N)c(C)c(=O)c2c1 ; Cc1cc([C@@H](C)Nc2ccc(Cl)nc2C(=O)NS(C)(=O)=O)c2oc(-c3cccc(F)c3C#N)c(C)c(=O)c2c1 ; Cc1cc([C@@H](C)Nc2ccc(Cl)nc2C(=O)NS(C)(=O)=O)c2oc(-c3ccccc3C#N)c(C)c(=O)c2c1] 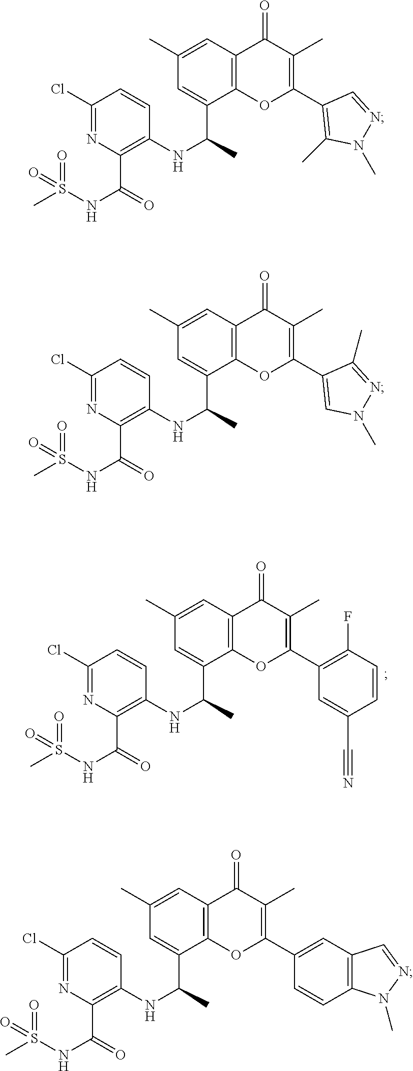 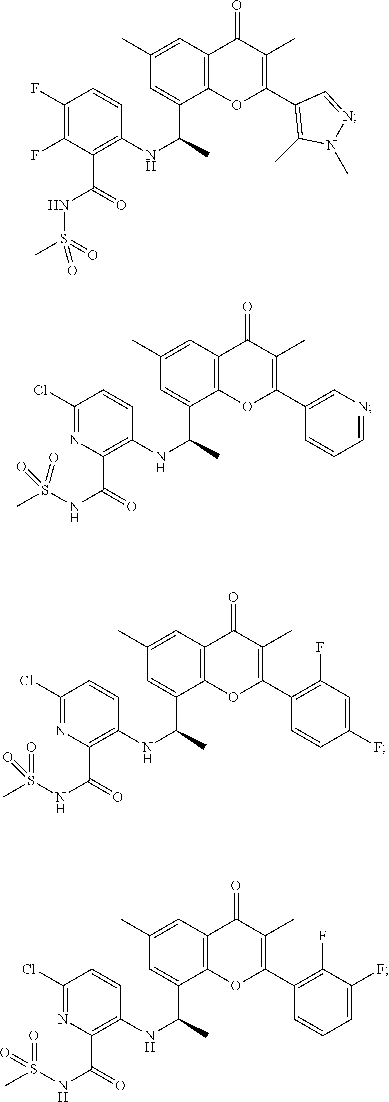 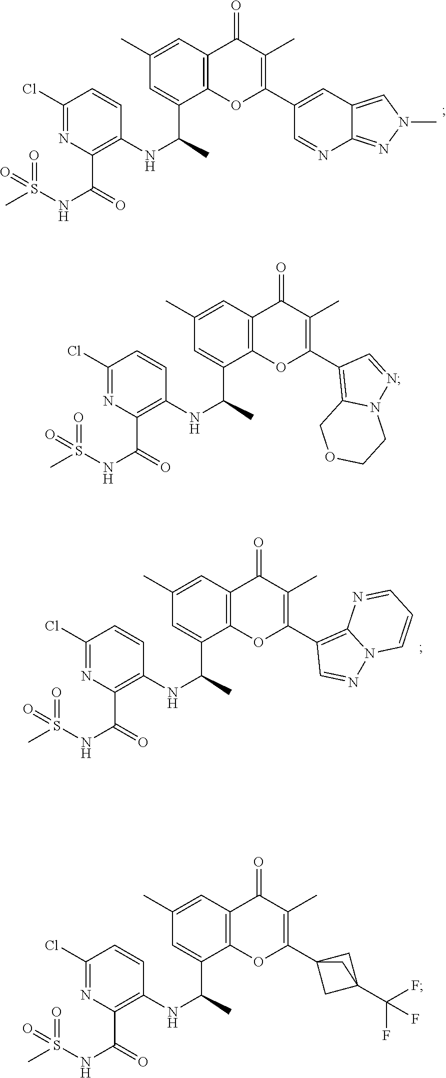 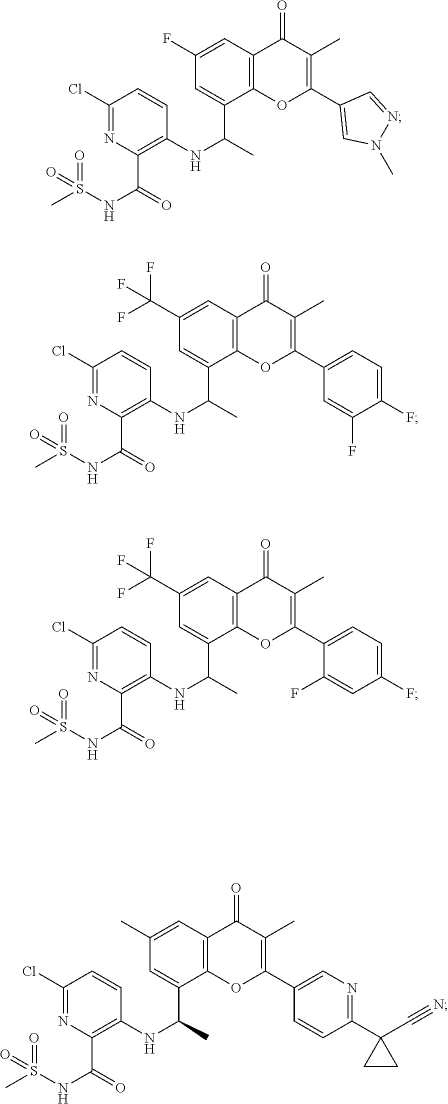 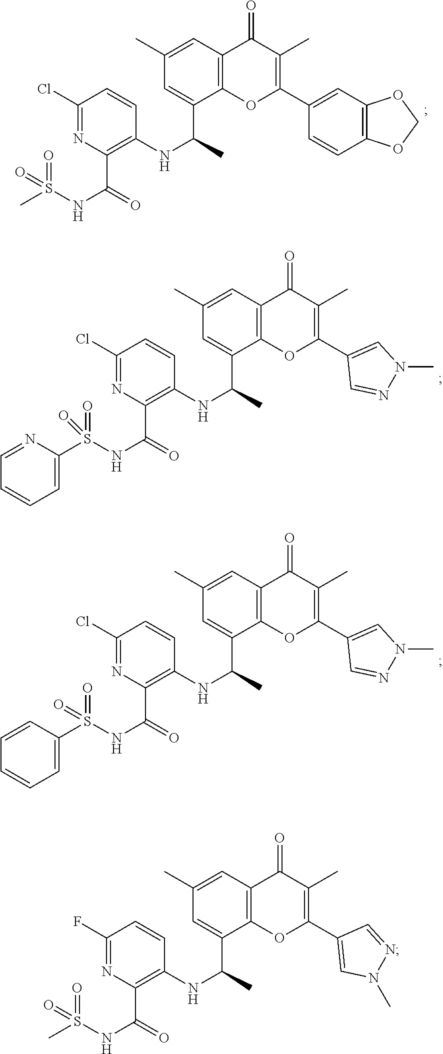 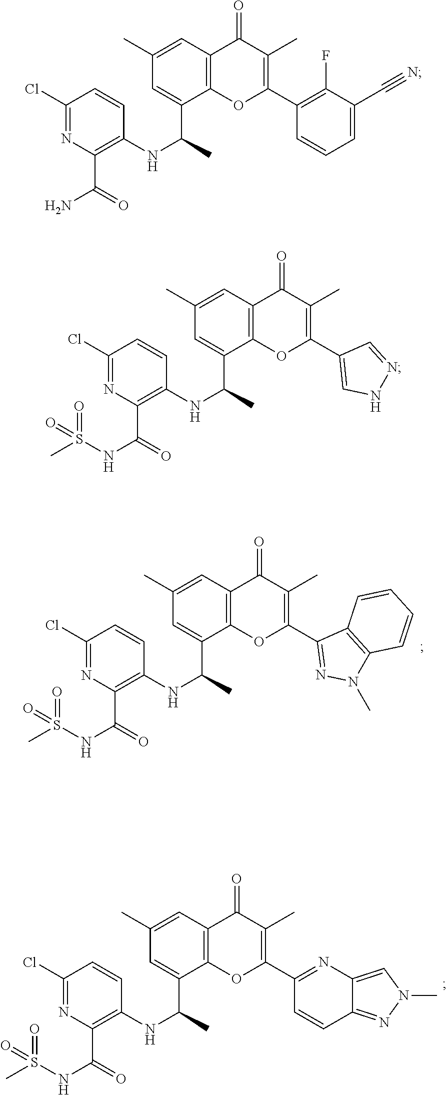 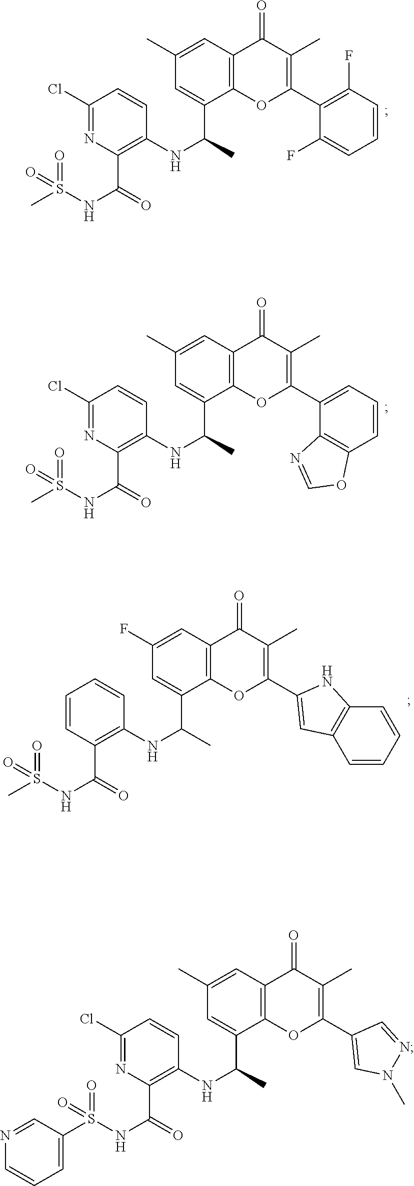 [SMILES: Cc1cc([C@@H](C)Nc2ccc(Cl)nc2C(=O)NS(C)(=O)=O)c2oc(-c3c(F)cccc3F)c(C)c(=O)c2c1 ; Cc1cc([C@@H](C)Nc2ccc(Cl)nc2C(=O)NS(C)(=O)=O)c2oc(-c3cccc4ocnc34)c(C)c(=O)c2c1 ; Cc1c(-c2cc3ccccc3[nH]2)oc2c(C(C)Nc3ccccc3C(=O)NS(C)(=O)=O)cc(F)cc2c1=O ; Cc1cc([C@@H](C)Nc2ccc(Cl)nc2C(=O)NS(=O)(=O)c2cccnc2)c2oc(-c3cnn(C)c3)c(C)c(=O)c2c1] 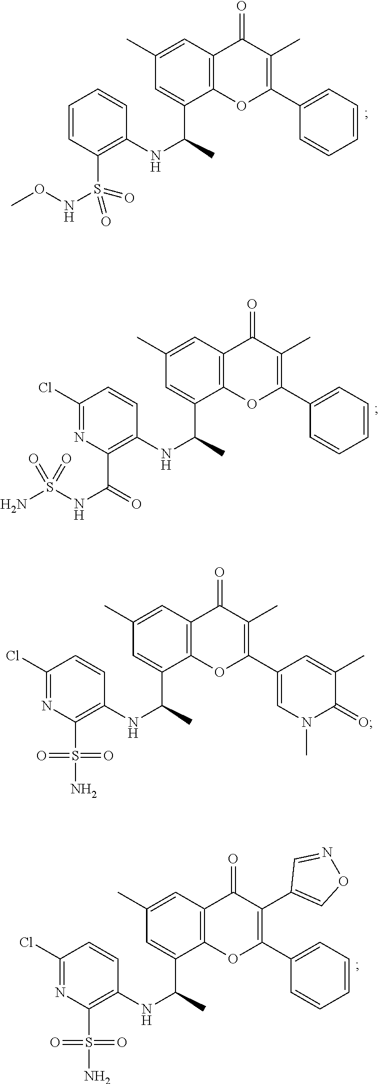 [SMILES: CONS(=O)(=O)c1ccccc1N[C@H](C)c1cc(C)cc2c(=O)c(C)c(-c3ccccc3)oc12 ; Cc1cc([C@@H](C)Nc2ccc(Cl)nc2C(=O)NS(N)(=O)=O)c2oc(-c3ccccc3)c(C)c(=O)c2c1 ; Cc1cc([C@@H](C)Nc2ccc(Cl)nc2S(N)(=O)=O)c2oc(-c3cc(C)c(=O)n(C)c3)c(C)c(=O)c2c1 ; Cc1cc([C@@H](C)Nc2ccc(Cl)nc2S(N)(=O)=O)c2oc(-c3ccccc3)c(-c3cnoc3)c(=O)c2c1] 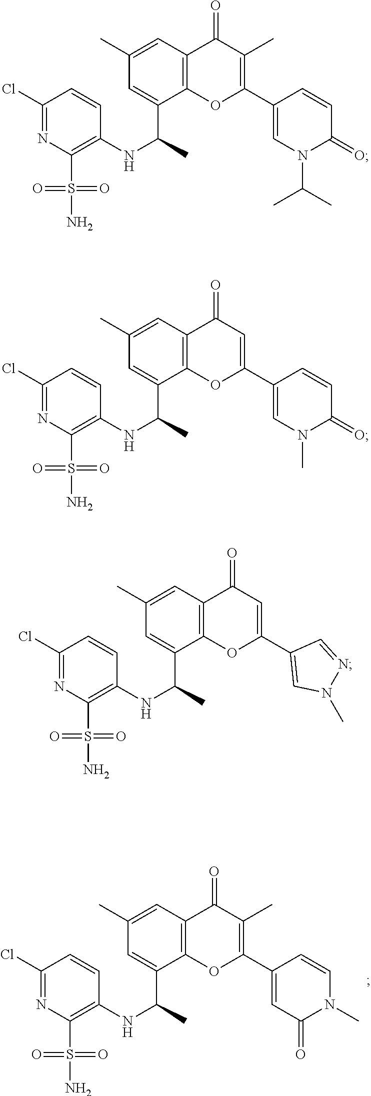 [SMILES: Cc1cc([C@@H](C)Nc2ccc(Cl)nc2S(N)(=O)=O)c2oc(-c3ccc(=O)n(C(C)C)c3)c(C)c(=O)c2c1 ; Cc1cc([C@@H](C)Nc2ccc(Cl)nc2S(N)(=O)=O)c2oc(-c3ccc(=O)n(C)c3)cc(=O)c2c1 ; Cc1cc([C@@H](C)Nc2ccc(Cl)nc2S(N)(=O)=O)c2oc(-c3cnn(C)c3)cc(=O)c2c1 ; Cc1cc([C@@H](C)Nc2ccc(Cl)nc2S(N)(=O)=O)c2oc(-c3ccn(C)c(=O)c3)c(C)c(=O)c2c1] 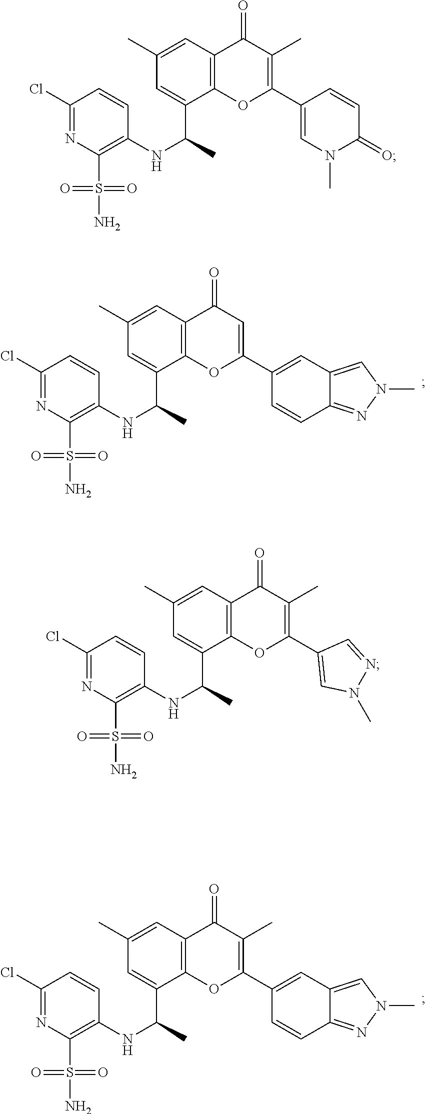 [SMILES: Cc1cc([C@@H](C)Nc2ccc(Cl)nc2S(N)(=O)=O)c2oc(-c3ccc(=O)n(C)c3)c(C)c(=O)c2c1 ; Cc1cc([C@@H](C)Nc2ccc(Cl)nc2S(N)(=O)=O)c2oc(-c3ccc4nn(C)cc4c3)cc(=O)c2c1 ; Cc1cc([C@@H](C)Nc2ccc(Cl)nc2S(N)(=O)=O)c2oc(-c3cnn(C)c3)c(C)c(=O)c2c1 ; Cc1cc([C@@H](C)Nc2ccc(Cl)nc2S(N)(=O)=O)c2oc(-c3ccc4nn(C)cc4c3)c(C)c(=O)c2c1] 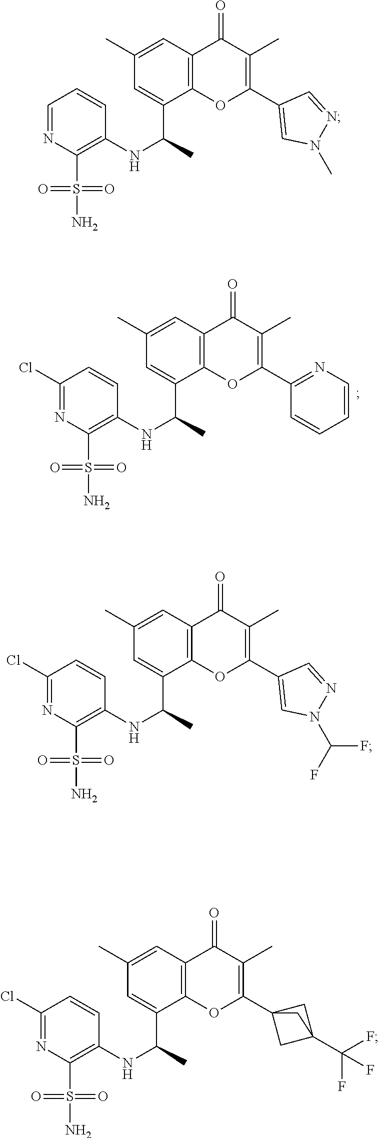 [SMILES: Cc1cc([C@@H](C)Nc2cccnc2S(N)(=O)=O)c2oc(-c3cnn(C)c3)c(C)c(=O)c2c1 ; Cc1cc([C@@H](C)Nc2ccc(Cl)nc2S(N)(=O)=O)c2oc(-c3ccccn3)c(C)c(=O)c2c1 ; Cc1cc([C@@H](C)Nc2ccc(Cl)nc2S(N)(=O)=O)c2oc(-c3cnn(C(F)F)c3)c(C)c(=O)c2c1 ; Cc1cc([C@@H](C)Nc2ccc(Cl)nc2S(N)(=O)=O)c2oc(C34CC(C(F)(F)F)(C3)C4)c(C)c(=O)c2c1] 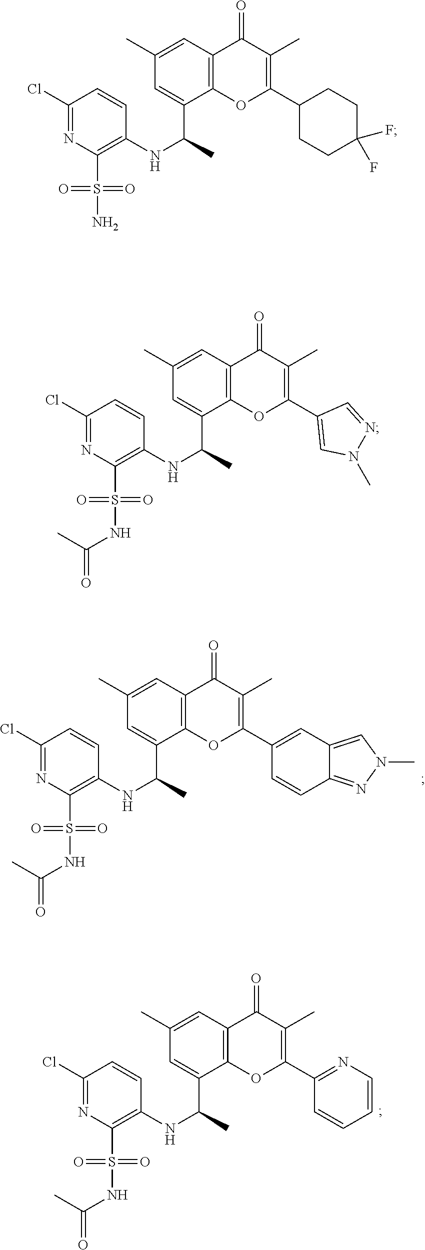 [SMILES: Cc1cc([C@@H](C)Nc2ccc(Cl)nc2S(N)(=O)=O)c2oc(C3CCC(F)(F)CC3)c(C)c(=O)c2c1 ; CC(=O)NS(=O)(=O)c1nc(Cl)ccc1N[C@H](C)c1cc(C)cc2c(=O)c(C)c(-c3cnn(C)c3)oc12 ; CC(=O)NS(=O)(=O)c1nc(Cl)ccc1N[C@H](C)c1cc(C)cc2c(=O)c(C)c(-c3ccc4nn(C)cc4c3)oc12 ; CC(=O)NS(=O)(=O)c1nc(Cl)ccc1N[C@H](C)c1cc(C)cc2c(=O)c(C)c(-c3ccccn3)oc12] 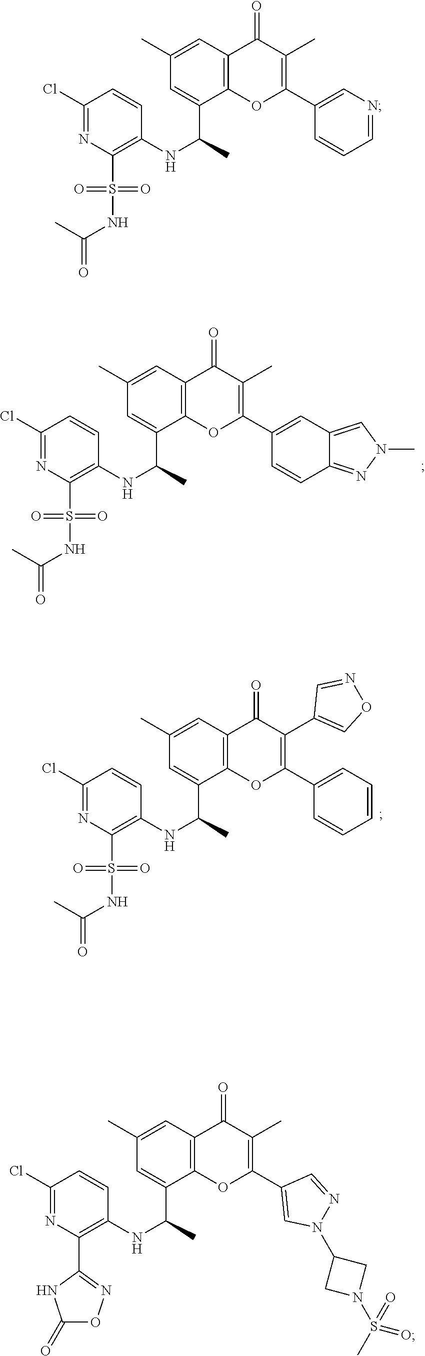 [SMILES: CC(=O)NS(=O)(=O)c1nc(Cl)ccc1N[C@H](C)c1cc(C)cc2c(=O)c(C)c(-c3cccnc3)oc12 ; CC(=O)NS(=O)(=O)c1nc(Cl)ccc1N[C@H](C)c1cc(C)cc2c(=O)c(C)c(-c3ccc4nn(C)cc4c3)oc12 ; CC(=O)NS(=O)(=O)c1nc(Cl)ccc1N[C@H](C)c1cc(C)cc2c(=O)c(-c3cnoc3)c(-c3ccccc3)oc12 ; Cc1cc([C@@H](C)Nc2ccc(Cl)nc2-c2noc(=O)[nH]2)c2oc(-c3cnn(C4CN(S(C)(=O)=O)C4)c3)c(C)c(=O)c2c1] 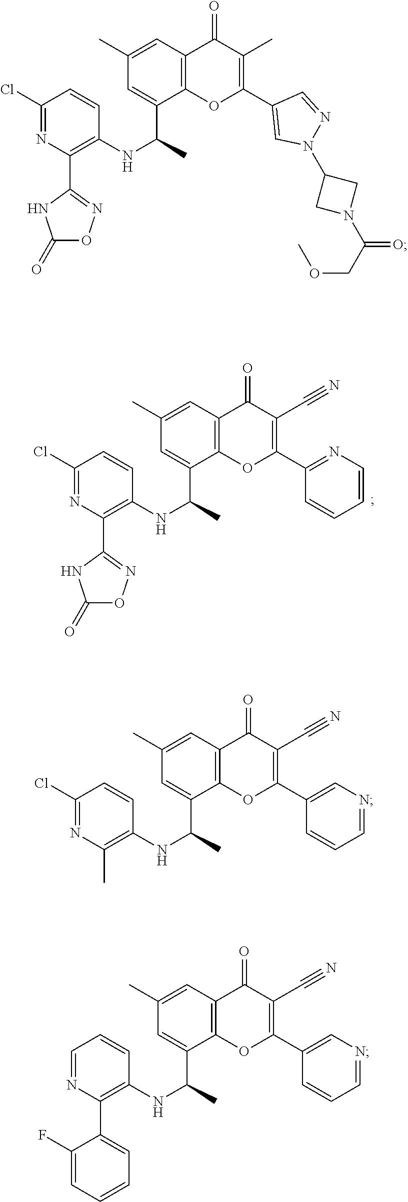 [SMILES: COCC(=O)N1CC(n2cc(-c3oc4c([C@@H](C)Nc5ccc(Cl)nc5-c5noc(=O)[nH]5)cc(C)cc4c(=O)c3C)cn2)C1 ; Cc1cc([C@@H](C)Nc2ccc(Cl)nc2-c2noc(=O)[nH]2)c2oc(-c3ccccn3)c(C#N)c(=O)c2c1 ; Cc1cc([C@@H](C)Nc2ccc(Cl)nc2C)c2oc(-c3cccnc3)c(C#N)c(=O)c2c1 ; Cc1cc([C@@H](C)Nc2cccnc2-c2ccccc2F)c2oc(-c3cccnc3)c(C#N)c(=O)c2c1] 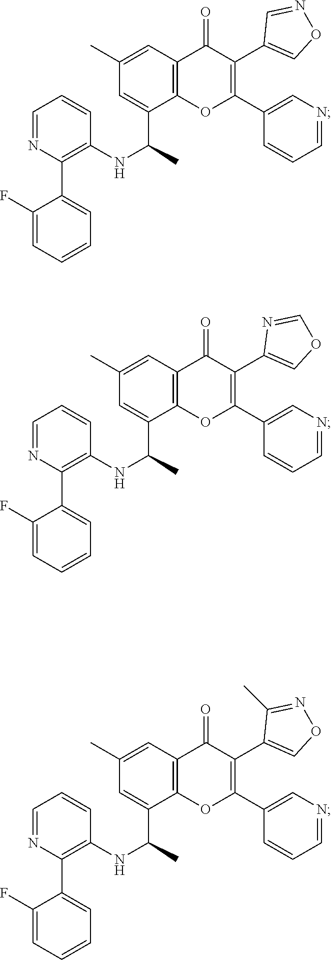 [SMILES: Cc1cc([C@@H](C)Nc2cccnc2-c2ccccc2F)c2oc(-c3cccnc3)c(-c3cnoc3)c(=O)c2c1 ; Cc1cc([C@@H](C)Nc2cccnc2-c2ccccc2F)c2oc(-c3cccnc3)c(-c3cocn3)c(=O)c2c1 ; Cc1cc([C@@H](C)Nc2cccnc2-c2ccccc2F)c2oc(-c3cccnc3)c(-c3conc3C)c(=O)c2c1] 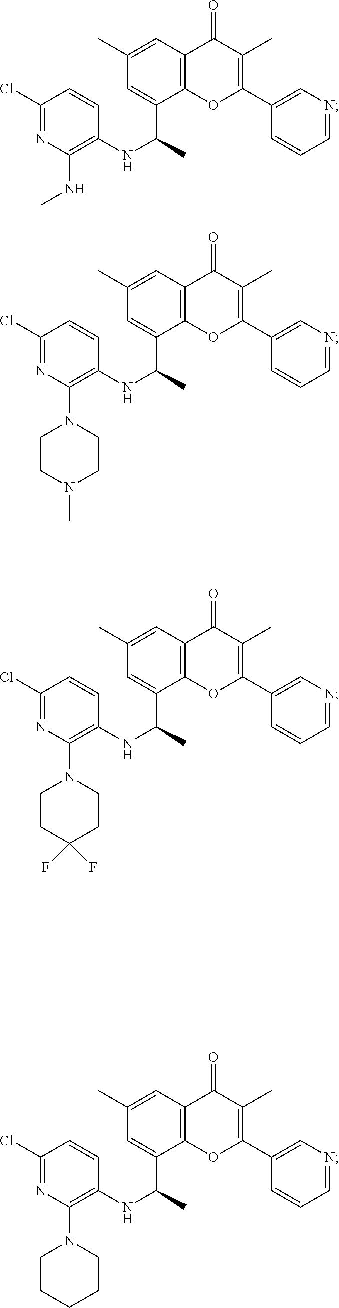 [SMILES: CNc1nc(Cl)ccc1N[C@H](C)c1cc(C)cc2c(=O)c(C)c(-c3cccnc3)oc12 ; Cc1cc([C@@H](C)Nc2ccc(Cl)nc2N2CCN(C)CC2)c2oc(-c3cccnc3)c(C)c(=O)c2c1 ; Cc1cc([C@@H](C)Nc2ccc(Cl)nc2N2CCC(F)(F)CC2)c2oc(-c3cccnc3)c(C)c(=O)c2c1 ; Cc1cc([C@@H](C)Nc2ccc(Cl)nc2N2CCCCC2)c2oc(-c3cccnc3)c(C)c(=O)c2c1] 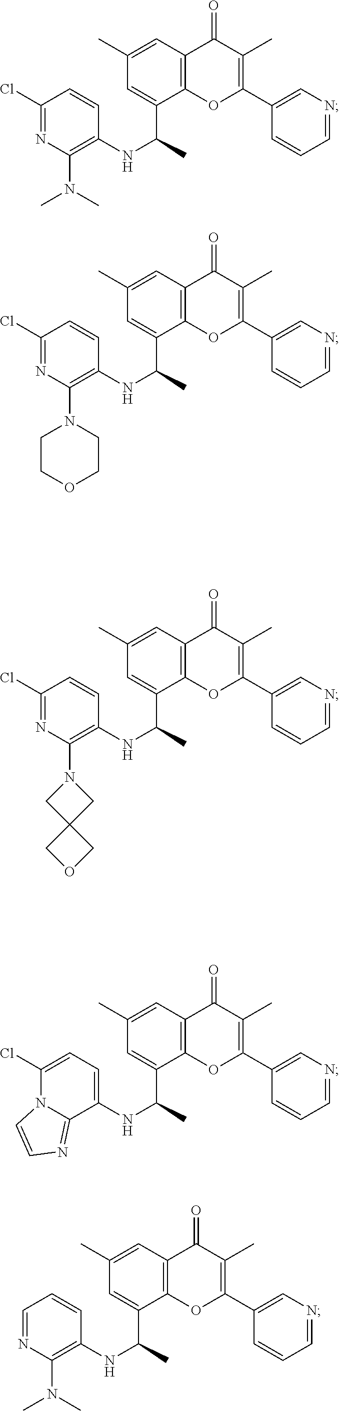 [SMILES: Cc1cc([C@@H](C)Nc2ccc(Cl)nc2N(C)C)c2oc(-c3cccnc3)c(C)c(=O)c2c1 ; Cc1cc([C@@H](C)Nc2ccc(Cl)nc2N2CCOCC2)c2oc(-c3cccnc3)c(C)c(=O)c2c1 ; Cc1cc([C@@H](C)Nc2ccc(Cl)nc2N2CC3(COC3)C2)c2oc(-c3cccnc3)c(C)c(=O)c2c1 ; Cc1cc([C@@H](C)Nc2ccc(Cl)n3ccnc23)c2oc(-c3cccnc3)c(C)c(=O)c2c1 ; Cc1cc([C@@H](C)Nc2cccnc2N(C)C)c2oc(-c3cccnc3)c(C)c(=O)c2c1] 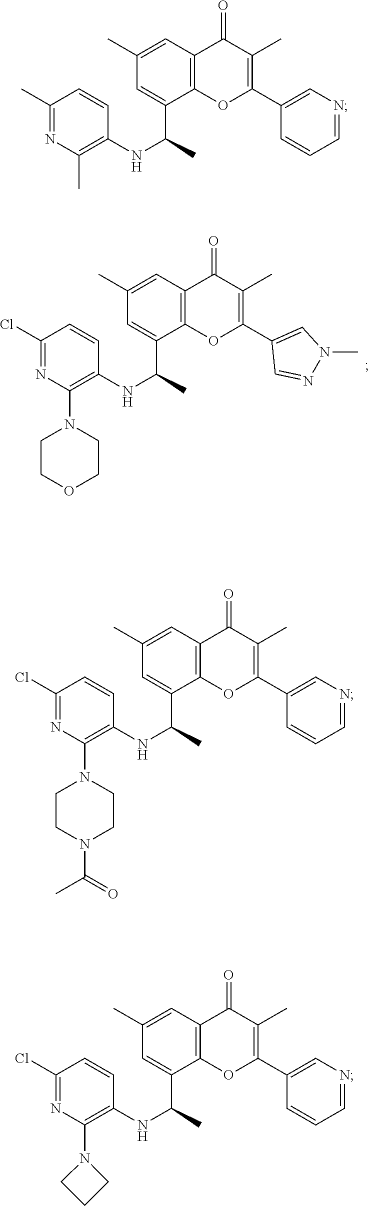 [SMILES: Cc1cc([C@@H](C)Nc2ccc(C)nc2C)c2oc(-c3cccnc3)c(C)c(=O)c2c1 ; Cc1cc([C@@H](C)Nc2ccc(Cl)nc2N2CCOCC2)c2oc(-c3cnn(C)c3)c(C)c(=O)c2c1 ; CC(=O)N1CCN(c2nc(Cl)ccc2N[C@H](C)c2cc(C)cc3c(=O)c(C)c(-c4cccnc4)oc23)CC1 ; Cc1cc([C@@H](C)Nc2ccc(Cl)nc2N2CCC2)c2oc(-c3cccnc3)c(C)c(=O)c2c1] 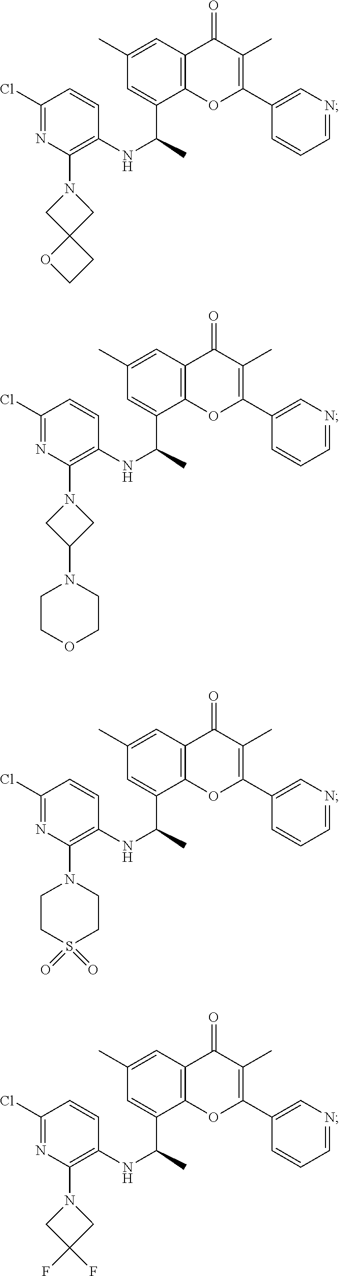 [SMILES: Cc1cc([C@@H](C)Nc2ccc(Cl)nc2N2CC3(CCO3)C2)c2oc(-c3cccnc3)c(C)c(=O)c2c1 ; Cc1cc([C@@H](C)Nc2ccc(Cl)nc2N2CC(N3CCOCC3)C2)c2oc(-c3cccnc3)c(C)c(=O)c2c1 ; Cc1cc([C@@H](C)Nc2ccc(Cl)nc2N2CCS(=O)(=O)CC2)c2oc(-c3cccnc3)c(C)c(=O)c2c1 ; Cc1cc([C@@H](C)Nc2ccc(Cl)nc2N2CC(F)(F)C2)c2oc(-c3cccnc3)c(C)c(=O)c2c1] 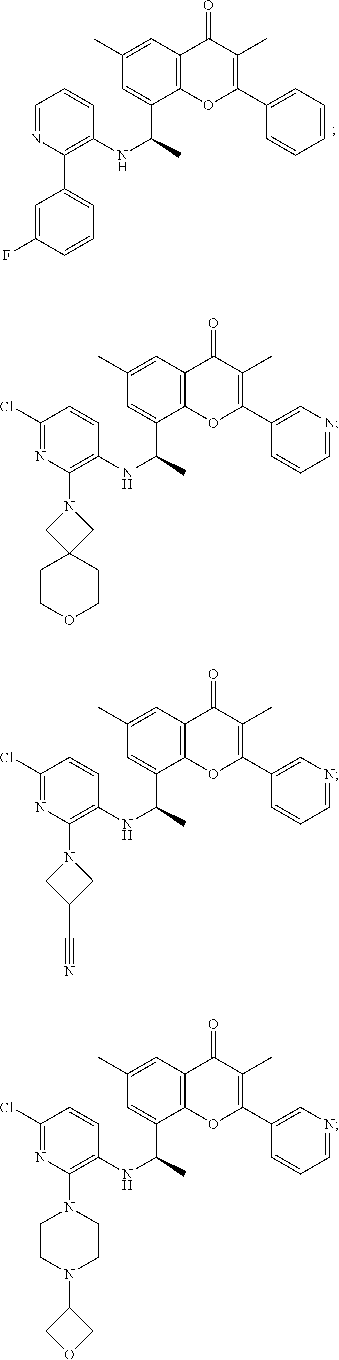 [SMILES: Cc1cc([C@@H](C)Nc2cccnc2-c2cccc(F)c2)c2oc(-c3ccccc3)c(C)c(=O)c2c1 ; Cc1cc([C@@H](C)Nc2ccc(Cl)nc2N2CC3(CCOCC3)C2)c2oc(-c3cccnc3)c(C)c(=O)c2c1 ; Cc1cc([C@@H](C)Nc2ccc(Cl)nc2N2CC(C#N)C2)c2oc(-c3cccnc3)c(C)c(=O)c2c1 ; Cc1cc([C@@H](C)Nc2ccc(Cl)nc2N2CCN(C3COC3)CC2)c2oc(-c3cccnc3)c(C)c(=O)c2c1] 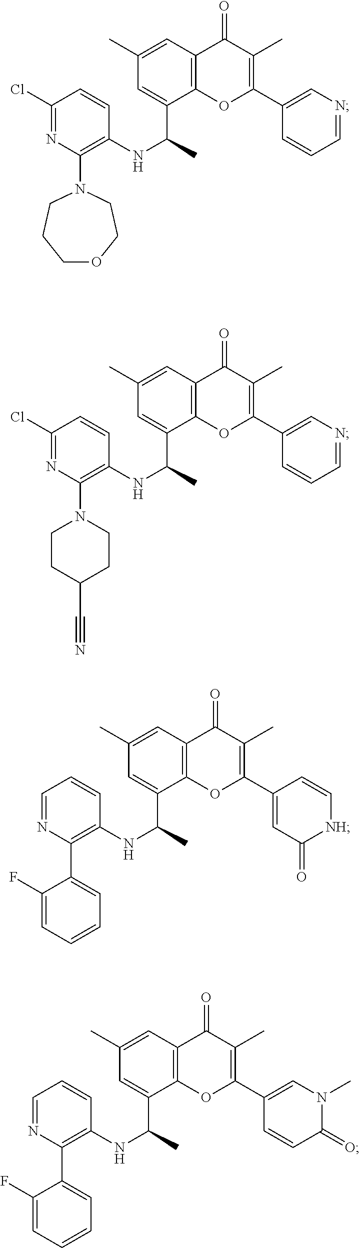 [SMILES: Cc1cc([C@@H](C)Nc2ccc(Cl)nc2N2CCCOCC2)c2oc(-c3cccnc3)c(C)c(=O)c2c1 ; Cc1cc([C@@H](C)Nc2ccc(Cl)nc2N2CCC(C#N)CC2)c2oc(-c3cccnc3)c(C)c(=O)c2c1 ; Cc1cc([C@@H](C)Nc2cccnc2-c2ccccc2F)c2oc(-c3cc[nH]c(=O)c3)c(C)c(=O)c2c1 ; Cc1cc([C@@H](C)Nc2cccnc2-c2ccccc2F)c2oc(-c3ccc(=O)n(C)c3)c(C)c(=O)c2c1] 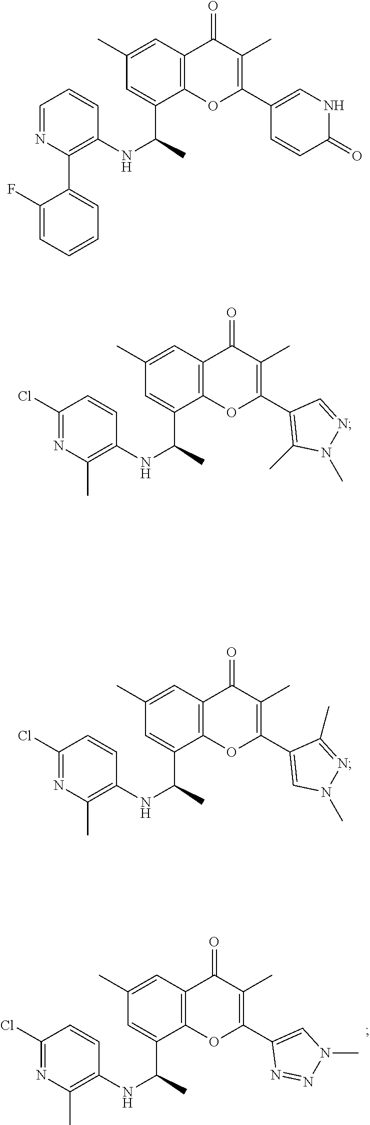 [SMILES: Cc1cc([C@@H](C)Nc2cccnc2-c2ccccc2F)c2oc(-c3ccc(=O)[nH]c3)c(C)c(=O)c2c1 ; Cc1cc([C@@H](C)Nc2ccc(Cl)nc2C)c2oc(-c3cnn(C)c3C)c(C)c(=O)c2c1 ; Cc1cc([C@@H](C)Nc2ccc(Cl)nc2C)c2oc(-c3cn(C)nc3C)c(C)c(=O)c2c1 ; Cc1cc([C@@H](C)Nc2ccc(Cl)nc2C)c2oc(-c3cn(C)nn3)c(C)c(=O)c2c1] 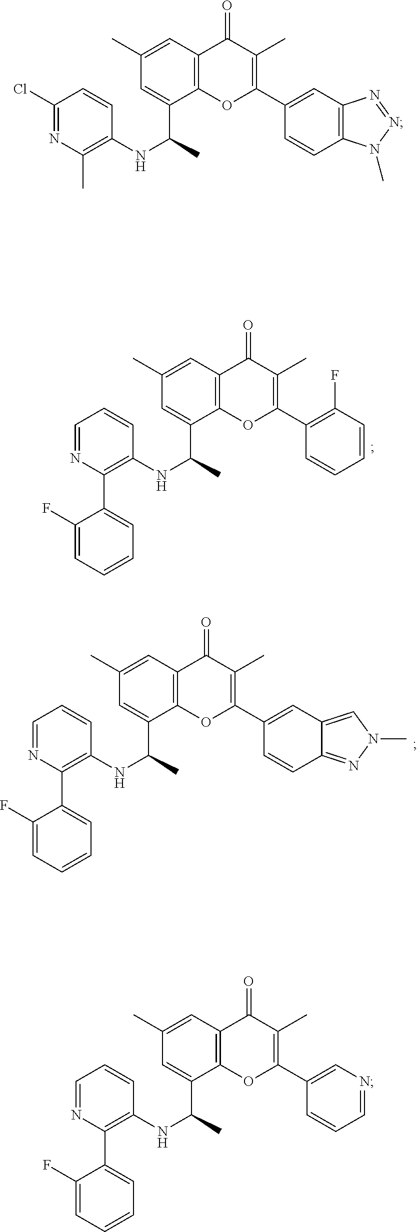 [SMILES: Cc1cc([C@@H](C)Nc2ccc(Cl)nc2C)c2oc(-c3ccc4c(c3)nnn4C)c(C)c(=O)c2c1 ; Cc1cc([C@@H](C)Nc2cccnc2-c2ccccc2F)c2oc(-c3ccccc3F)c(C)c(=O)c2c1 ; Cc1cc([C@@H](C)Nc2cccnc2-c2ccccc2F)c2oc(-c3ccc4nn(C)cc4c3)c(C)c(=O)c2c1 ; Cc1cc([C@@H](C)Nc2cccnc2-c2ccccc2F)c2oc(-c3cccnc3)c(C)c(=O)c2c1] 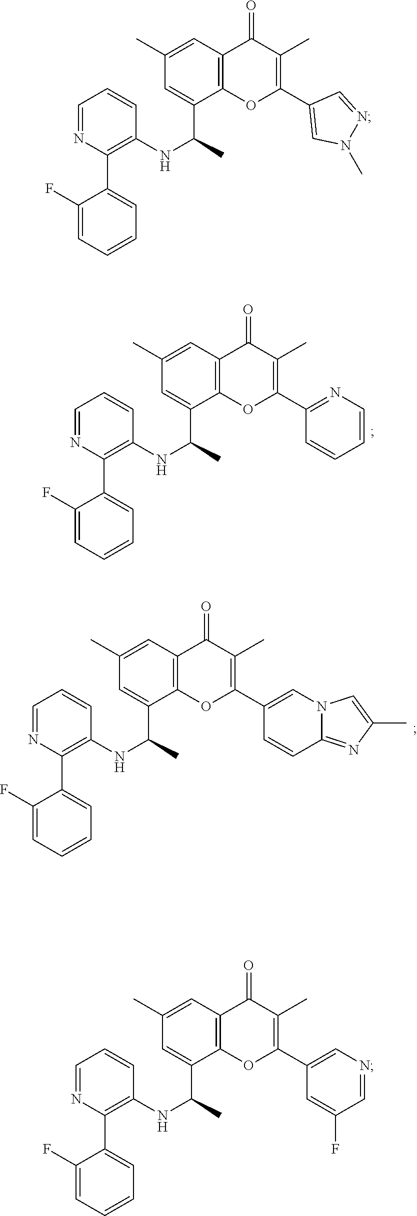 [SMILES: Cc1cc([C@@H](C)Nc2cccnc2-c2ccccc2F)c2oc(-c3cnn(C)c3)c(C)c(=O)c2c1 ; Cc1cc([C@@H](C)Nc2cccnc2-c2ccccc2F)c2oc(-c3ccccn3)c(C)c(=O)c2c1 ; Cc1cc([C@@H](C)Nc2cccnc2-c2ccccc2F)c2oc(-c3ccc4nc(C)cn4c3)c(C)c(=O)c2c1 ; Cc1cc([C@@H](C)Nc2cccnc2-c2ccccc2F)c2oc(-c3cncc(F)c3)c(C)c(=O)c2c1] 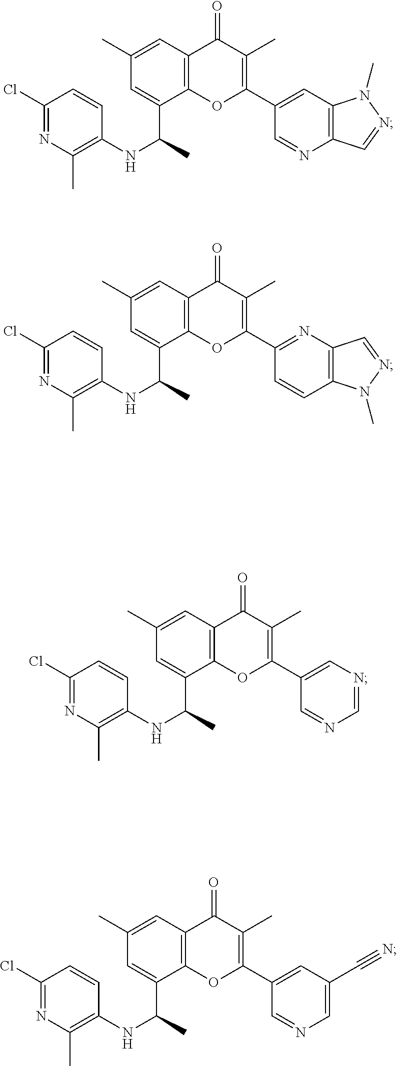 [SMILES: Cc1cc([C@@H](C)Nc2ccc(Cl)nc2C)c2oc(-c3cnc4cnn(C)c4c3)c(C)c(=O)c2c1 ; Cc1cc([C@@H](C)Nc2ccc(Cl)nc2C)c2oc(-c3ccc4c(cnn4C)n3)c(C)c(=O)c2c1 ; Cc1cc([C@@H](C)Nc2ccc(Cl)nc2C)c2oc(-c3cncnc3)c(C)c(=O)c2c1 ; Cc1cc([C@@H](C)Nc2ccc(Cl)nc2C)c2oc(-c3cncc(C#N)c3)c(C)c(=O)c2c1] 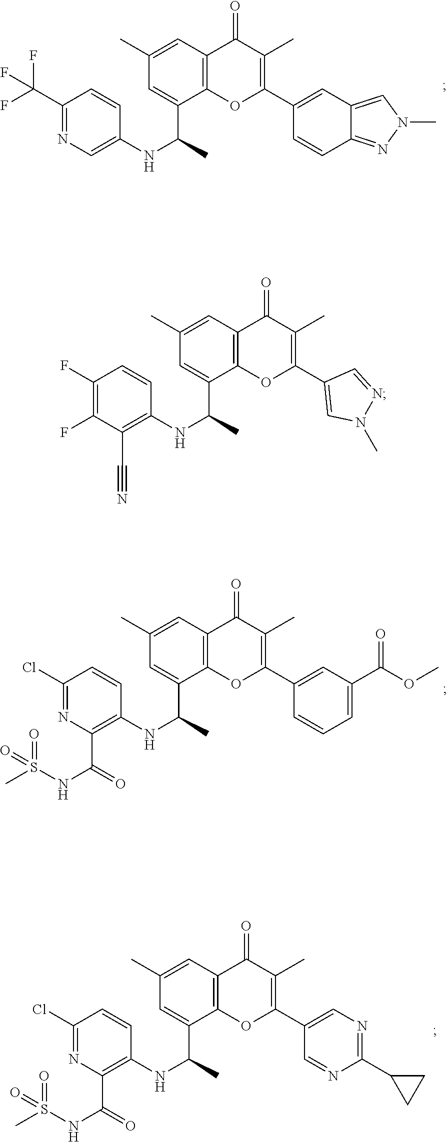 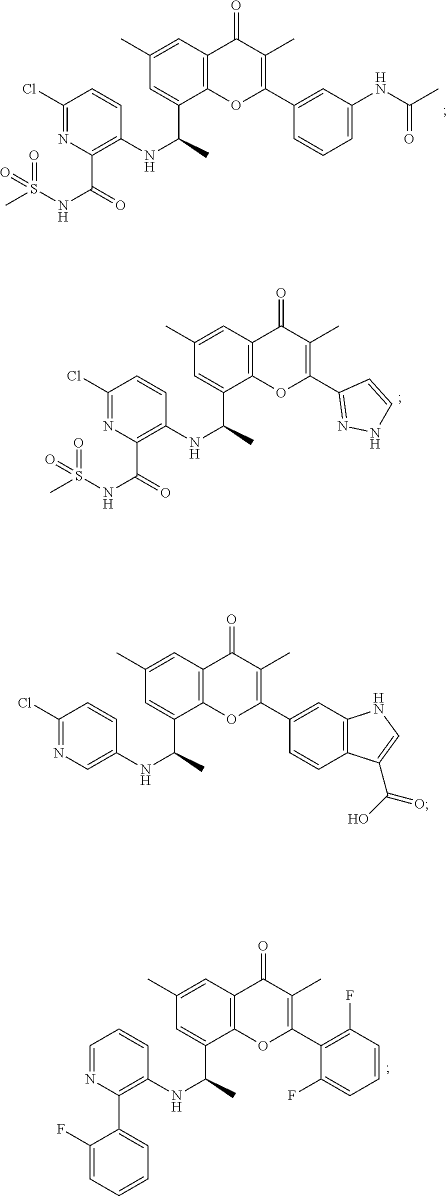 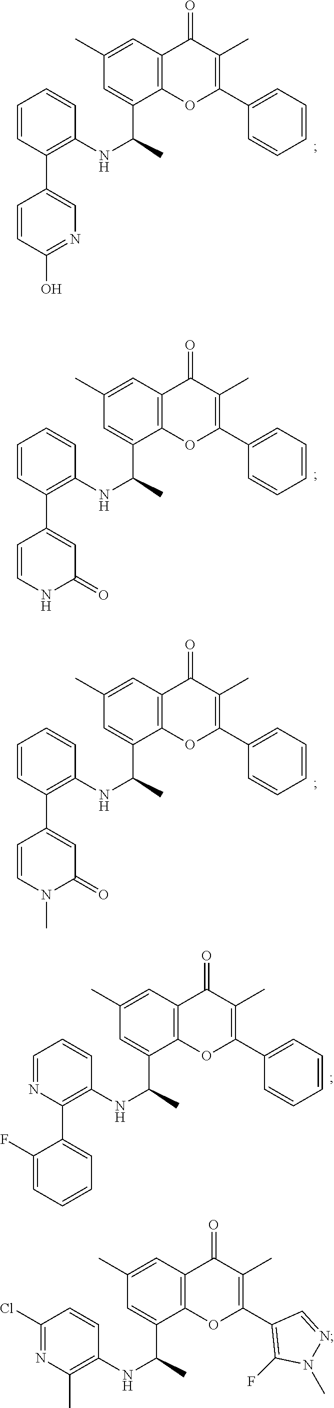 [SMILES: Cc1cc([C@@H](C)Nc2ccccc2-c2ccc(O)nc2)c2oc(-c3ccccc3)c(C)c(=O)c2c1 ; Cc1cc([C@@H](C)Nc2ccccc2-c2cc[nH]c(=O)c2)c2oc(-c3ccccc3)c(C)c(=O)c2c1 ; Cc1cc([C@@H](C)Nc2ccccc2-c2ccn(C)c(=O)c2)c2oc(-c3ccccc3)c(C)c(=O)c2c1 ; Cc1cc([C@@H](C)Nc2cccnc2-c2ccccc2F)c2oc(-c3ccccc3)c(C)c(=O)c2c1 ; Cc1cc([C@@H](C)Nc2ccc(Cl)nc2C)c2oc(-c3cnn(C)c3F)c(C)c(=O)c2c1] 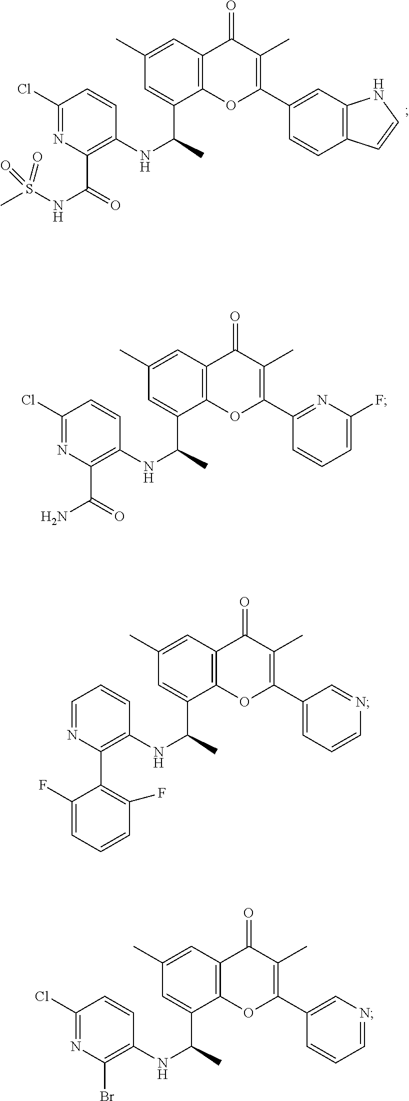 [SMILES: Cc1cc([C@@H](C)Nc2ccc(Cl)nc2C(=O)NS(C)(=O)=O)c2oc(-c3ccc4cc[nH]c4c3)c(C)c(=O)c2c1 ; Cc1cc([C@@H](C)Nc2ccc(Cl)nc2C(N)=O)c2oc(-c3cccc(F)n3)c(C)c(=O)c2c1 ; Cc1cc([C@@H](C)Nc2cccnc2-c2c(F)cccc2F)c2oc(-c3cccnc3)c(C)c(=O)c2c1 ; Cc1cc([C@@H](C)Nc2ccc(Cl)nc2Br)c2oc(-c3cccnc3)c(C)c(=O)c2c1] 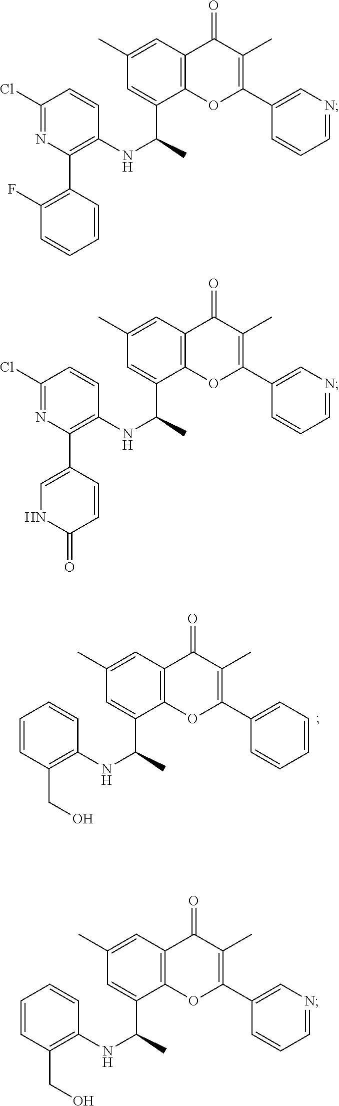 [SMILES: Cc1cc([C@@H](C)Nc2ccc(Cl)nc2-c2ccccc2F)c2oc(-c3cccnc3)c(C)c(=O)c2c1 ; Cc1cc([C@@H](C)Nc2ccc(Cl)nc2-c2ccc(=O)[nH]c2)c2oc(-c3cccnc3)c(C)c(=O)c2c1 ; Cc1cc([C@@H](C)Nc2ccccc2CO)c2oc(-c3ccccc3)c(C)c(=O)c2c1 ; Cc1cc([C@@H](C)Nc2ccccc2CO)c2oc(-c3cccnc3)c(C)c(=O)c2c1] 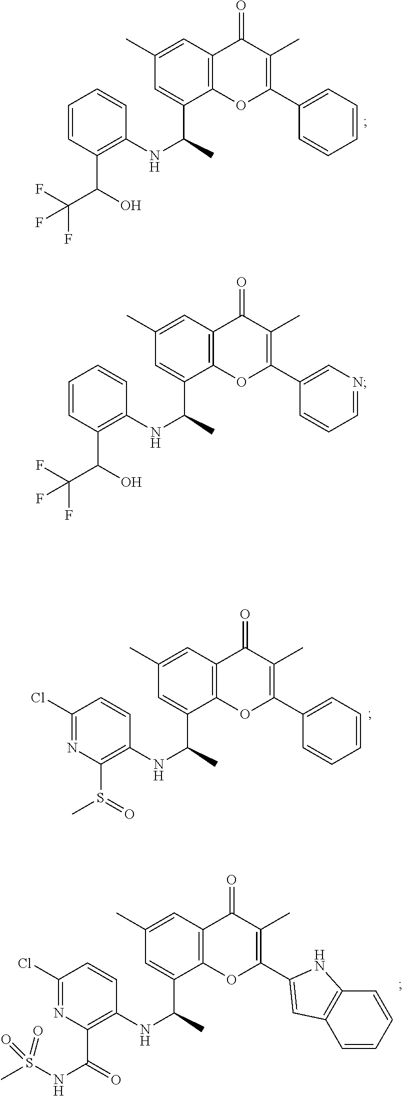 [SMILES: Cc1cc([C@@H](C)Nc2ccccc2C(O)C(F)(F)F)c2oc(-c3ccccc3)c(C)c(=O)c2c1 ; Cc1cc([C@@H](C)Nc2ccccc2C(O)C(F)(F)F)c2oc(-c3cccnc3)c(C)c(=O)c2c1 ; Cc1cc([C@@H](C)Nc2ccc(Cl)nc2S(C)=O)c2oc(-c3ccccc3)c(C)c(=O)c2c1 ; Cc1cc([C@@H](C)Nc2ccc(Cl)nc2C(=O)NS(C)(=O)=O)c2oc(-c3cc4ccccc4[nH]3)c(C)c(=O)c2c1] 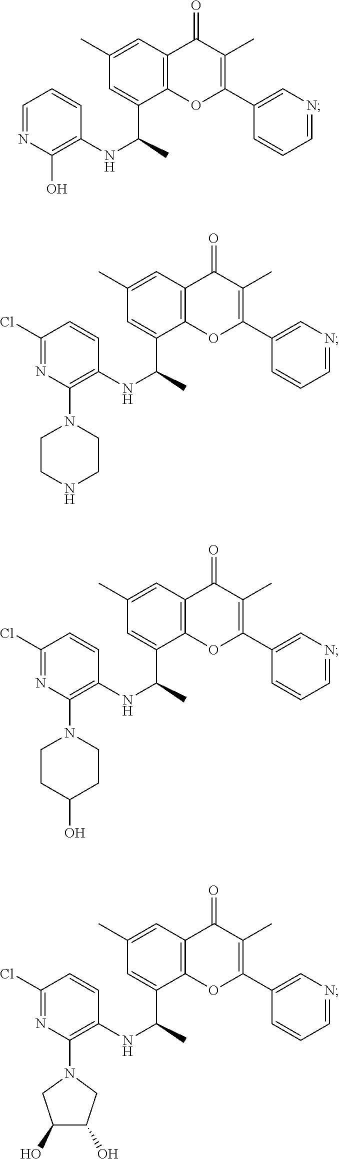 [SMILES: Cc1cc([C@@H](C)Nc2cccnc2O)c2oc(-c3cccnc3)c(C)c(=O)c2c1 ; Cc1cc([C@@H](C)Nc2ccc(Cl)nc2N2CCNCC2)c2oc(-c3cccnc3)c(C)c(=O)c2c1 ; Cc1cc([C@@H](C)Nc2ccc(Cl)nc2N2CCC(O)CC2)c2oc(-c3cccnc3)c(C)c(=O)c2c1 ; Cc1cc([C@@H](C)Nc2ccc(Cl)nc2N2C[C@H](O)[C@@H](O)C2)c2oc(-c3cccnc3)c(C)c(=O)c2c1] 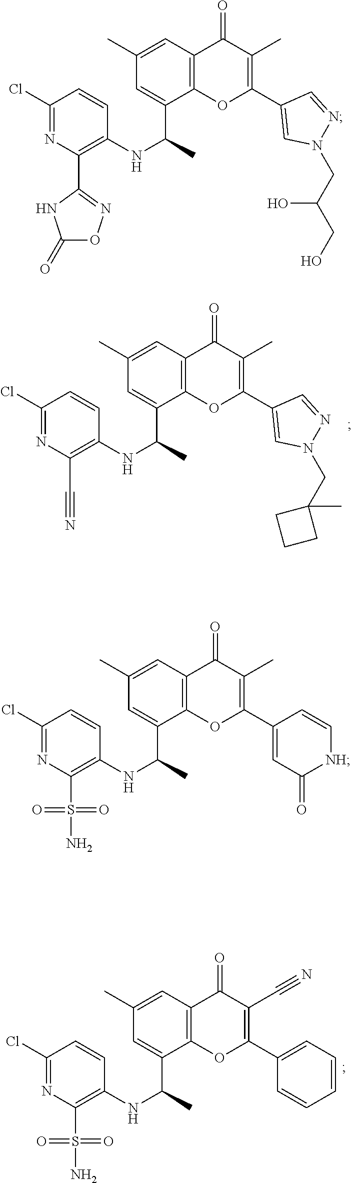 [SMILES: Cc1cc([C@@H](C)Nc2ccc(Cl)nc2-c2noc(=O)[nH]2)c2oc(-c3cnn(CC(O)CO)c3)c(C)c(=O)c2c1 ; Cc1cc([C@@H](C)Nc2ccc(Cl)nc2C#N)c2oc(-c3cnn(CC4(C)CCC4)c3)c(C)c(=O)c2c1 ; Cc1cc([C@@H](C)Nc2ccc(Cl)nc2S(N)(=O)=O)c2oc(-c3cc[nH]c(=O)c3)c(C)c(=O)c2c1 ; Cc1cc([C@@H](C)Nc2ccc(Cl)nc2S(N)(=O)=O)c2oc(-c3ccccc3)c(C#N)c(=O)c2c1] 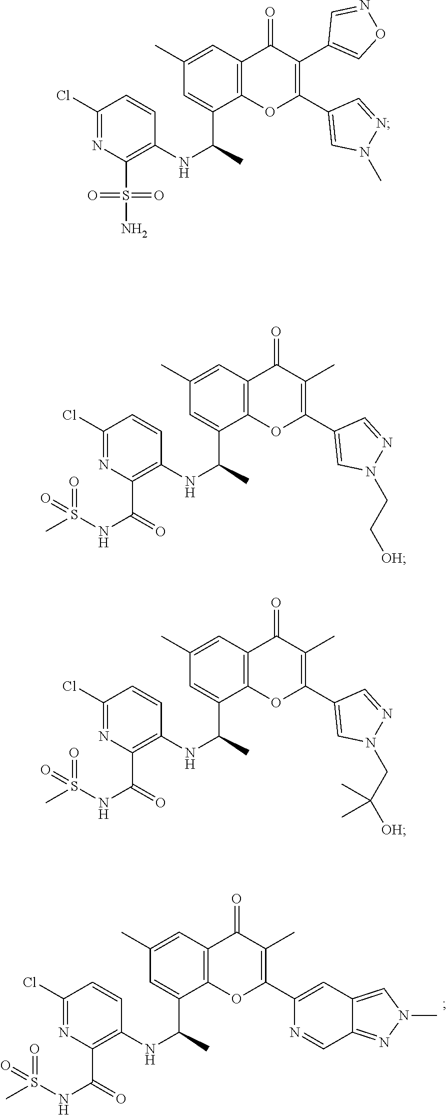 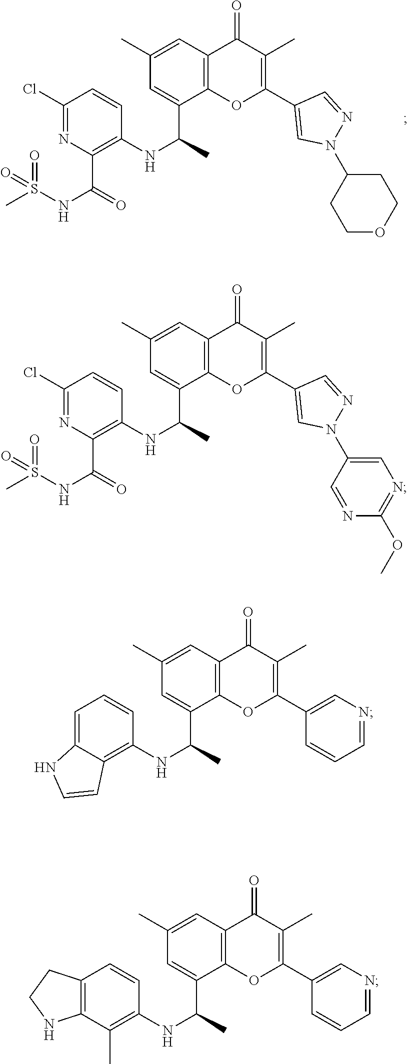 [SMILES: Cc1cc([C@@H](C)Nc2ccc(Cl)nc2C(=O)NS(C)(=O)=O)c2oc(-c3cnn(C4CCOCC4)c3)c(C)c(=O)c2c1 ; COc1ncc(-n2cc(-c3oc4c([C@@H](C)Nc5ccc(Cl)nc5C(=O)NS(C)(=O)=O)cc(C)cc4c(=O)c3C)cn2)cn1 ; Cc1cc([C@@H](C)Nc2cccc3[nH]ccc23)c2oc(-c3cccnc3)c(C)c(=O)c2c1 ; Cc1cc([C@@H](C)Nc2ccc3c(c2C)NCC3)c2oc(-c3cccnc3)c(C)c(=O)c2c1] 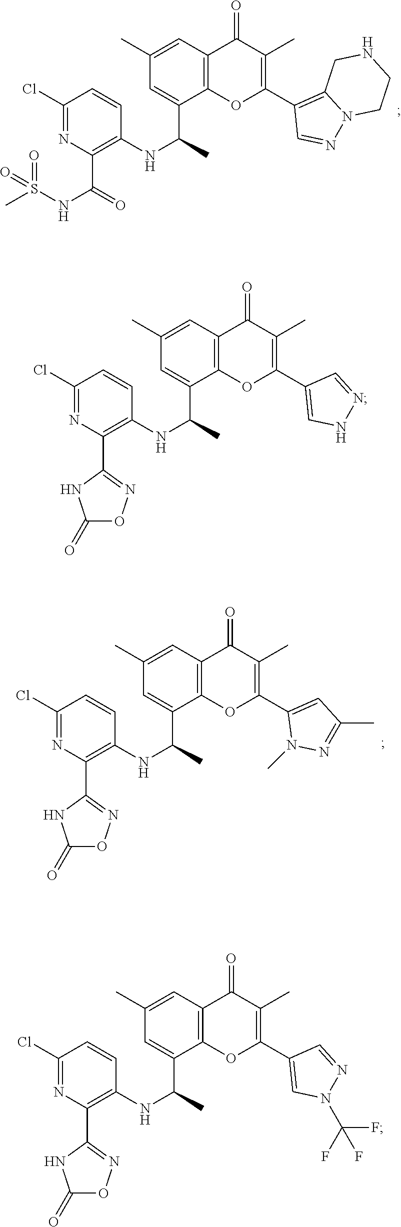 [SMILES: Cc1cc([C@@H](C)Nc2ccc(Cl)nc2C(=O)NS(C)(=O)=O)c2oc(-c3cnn4c3CNCC4)c(C)c(=O)c2c1 ; Cc1cc([C@@H](C)Nc2ccc(Cl)nc2-c2noc(=O)[nH]2)c2oc(-c3cn[nH]c3)c(C)c(=O)c2c1 ; Cc1cc([C@@H](C)Nc2ccc(Cl)nc2-c2noc(=O)[nH]2)c2oc(-c3cc(C)nn3C)c(C)c(=O)c2c1 ; Cc1cc([C@@H](C)Nc2ccc(Cl)nc2-c2noc(=O)[nH]2)c2oc(-c3cnn(C(F)(F)F)c3)c(C)c(=O)c2c1] 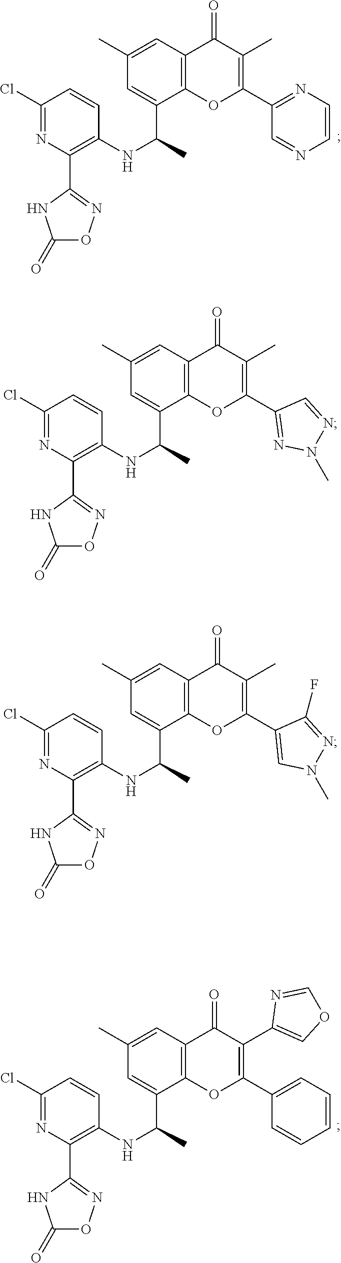 [SMILES: Cc1cc([C@@H](C)Nc2ccc(Cl)nc2-c2noc(=O)[nH]2)c2oc(-c3cnccn3)c(C)c(=O)c2c1 ; Cc1cc([C@@H](C)Nc2ccc(Cl)nc2-c2noc(=O)[nH]2)c2oc(-c3cnn(C)n3)c(C)c(=O)c2c1 ; Cc1cc([C@@H](C)Nc2ccc(Cl)nc2-c2noc(=O)[nH]2)c2oc(-c3cn(C)nc3F)c(C)c(=O)c2c1 ; Cc1cc([C@@H](C)Nc2ccc(Cl)nc2-c2noc(=O)[nH]2)c2oc(-c3ccccc3)c(-c3cocn3)c(=O)c2c1] 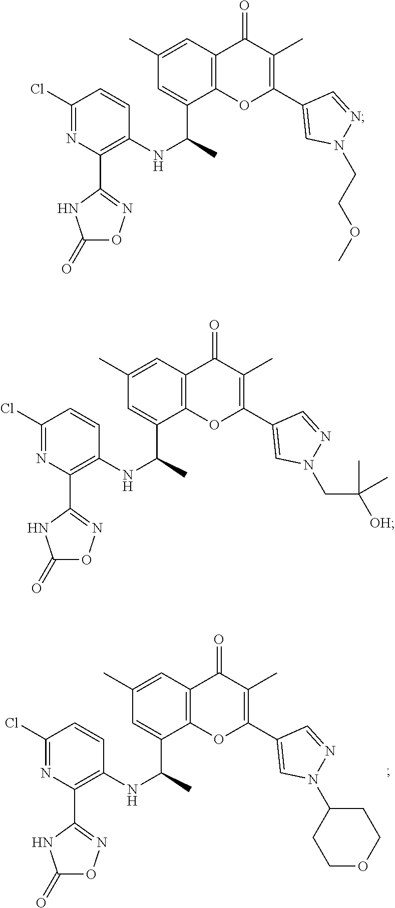 [SMILES: COCCn1cc(-c2oc3c([C@@H](C)Nc4ccc(Cl)nc4-c4noc(=O)[nH]4)cc(C)cc3c(=O)c2C)cn1 ; Cc1cc([C@@H](C)Nc2ccc(Cl)nc2-c2noc(=O)[nH]2)c2oc(-c3cnn(CC(C)(C)O)c3)c(C)c(=O)c2c1 ; Cc1cc([C@@H](C)Nc2ccc(Cl)nc2-c2noc(=O)[nH]2)c2oc(-c3cnn(C4CCOCC4)c3)c(C)c(=O)c2c1] 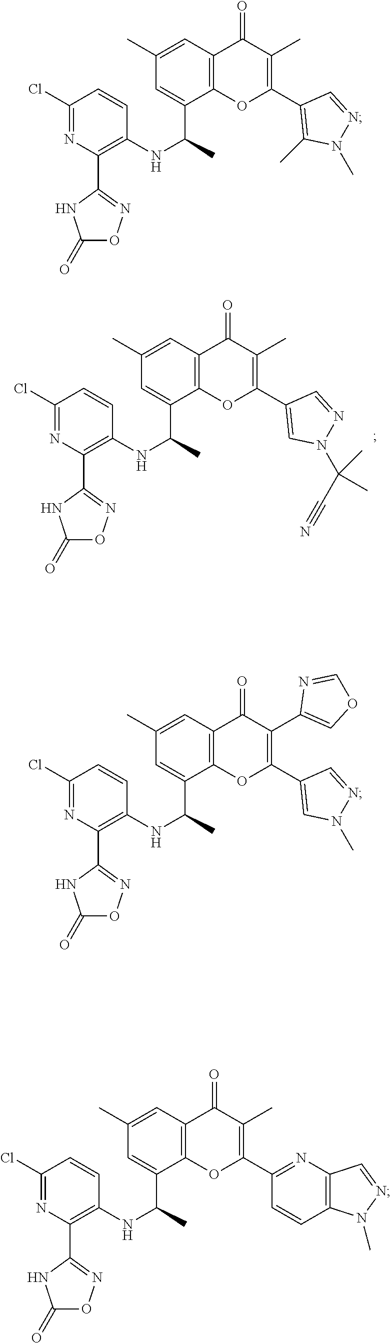 [SMILES: Cc1cc([C@@H](C)Nc2ccc(Cl)nc2-c2noc(=O)[nH]2)c2oc(-c3cnn(C)c3C)c(C)c(=O)c2c1 ; Cc1cc([C@@H](C)Nc2ccc(Cl)nc2-c2noc(=O)[nH]2)c2oc(-c3cnn(C(C)(C)C#N)c3)c(C)c(=O)c2c1 ; Cc1cc([C@@H](C)Nc2ccc(Cl)nc2-c2noc(=O)[nH]2)c2oc(-c3cnn(C)c3)c(-c3cocn3)c(=O)c2c1 ; Cc1cc([C@@H](C)Nc2ccc(Cl)nc2-c2noc(=O)[nH]2)c2oc(-c3ccc4c(cnn4C)n3)c(C)c(=O)c2c1] 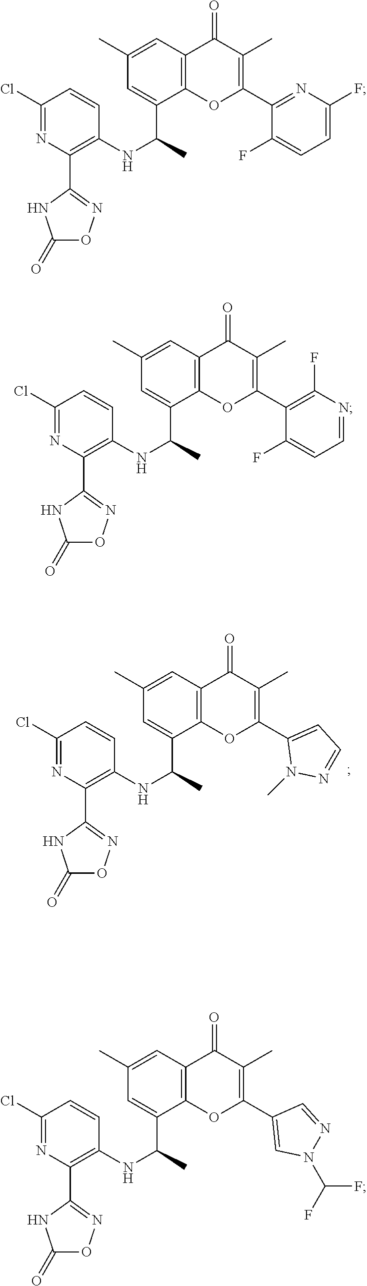 [SMILES: Cc1cc([C@@H](C)Nc2ccc(Cl)nc2-c2noc(=O)[nH]2)c2oc(-c3nc(F)ccc3F)c(C)c(=O)c2c1 ; Cc1cc([C@@H](C)Nc2ccc(Cl)nc2-c2noc(=O)[nH]2)c2oc(-c3c(F)ccnc3F)c(C)c(=O)c2c1 ; Cc1cc([C@@H](C)Nc2ccc(Cl)nc2-c2noc(=O)[nH]2)c2oc(-c3ccnn3C)c(C)c(=O)c2c1 ; Cc1cc([C@@H](C)Nc2ccc(Cl)nc2-c2noc(=O)[nH]2)c2oc(-c3cnn(C(F)F)c3)c(C)c(=O)c2c1] 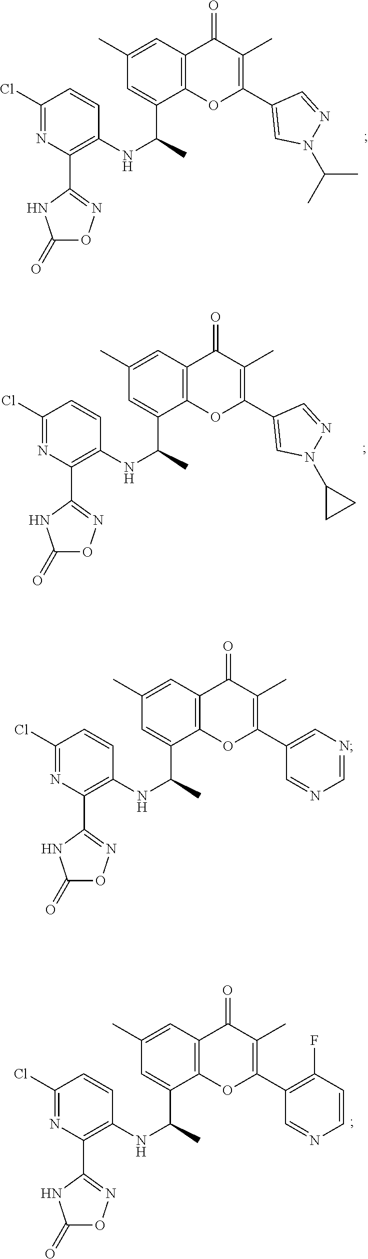 [SMILES: Cc1cc([C@@H](C)Nc2ccc(Cl)nc2-c2noc(=O)[nH]2)c2oc(-c3cnn(C(C)C)c3)c(C)c(=O)c2c1 ; Cc1cc([C@@H](C)Nc2ccc(Cl)nc2-c2noc(=O)[nH]2)c2oc(-c3cnn(C4CC4)c3)c(C)c(=O)c2c1 ; Cc1cc([C@@H](C)Nc2ccc(Cl)nc2-c2noc(=O)[nH]2)c2oc(-c3cncnc3)c(C)c(=O)c2c1 ; Cc1cc([C@@H](C)Nc2ccc(Cl)nc2-c2noc(=O)[nH]2)c2oc(-c3cnccc3F)c(C)c(=O)c2c1] 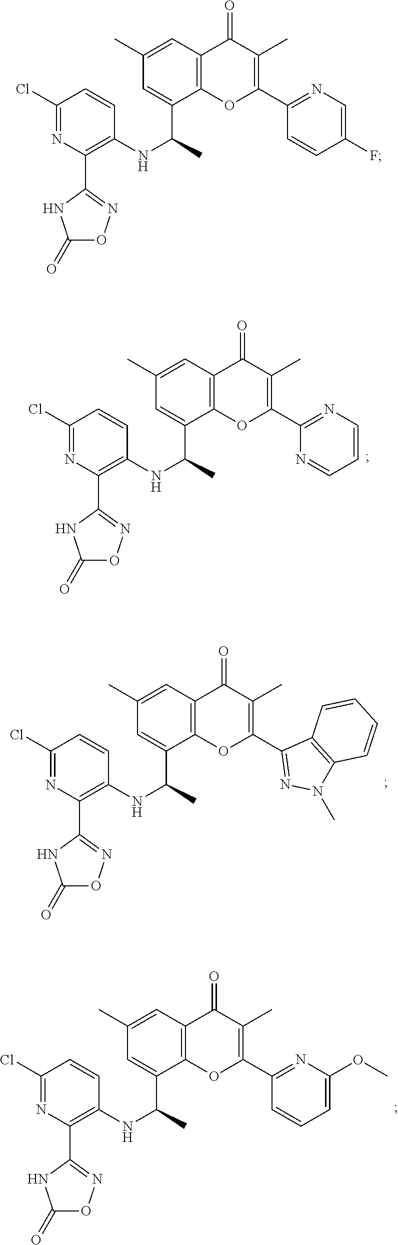 [SMILES: Cc1cc([C@@H](C)Nc2ccc(Cl)nc2-c2noc(=O)[nH]2)c2oc(-c3ccc(F)cn3)c(C)c(=O)c2c1 ; Cc1cc([C@@H](C)Nc2ccc(Cl)nc2-c2noc(=O)[nH]2)c2oc(-c3ncccn3)c(C)c(=O)c2c1 ; Cc1cc([C@@H](C)Nc2ccc(Cl)nc2-c2noc(=O)[nH]2)c2oc(-c3nn(C)c4ccccc34)c(C)c(=O)c2c1 ; COc1cccc(-c2oc3c([C@@H](C)Nc4ccc(Cl)nc4-c4noc(=O)[nH]4)cc(C)cc3c(=O)c2C)n1] 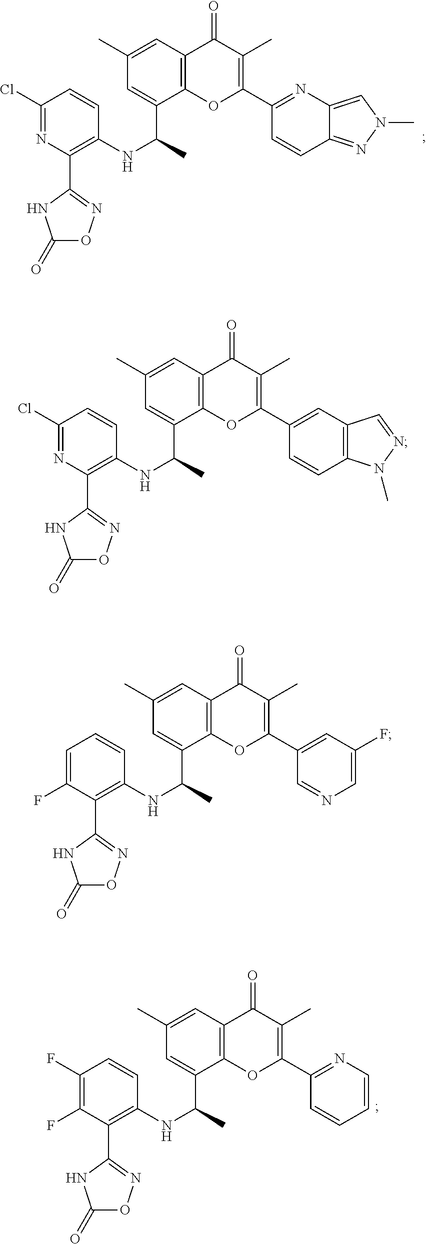 [SMILES: Cc1cc([C@@H](C)Nc2ccc(Cl)nc2-c2noc(=O)[nH]2)c2oc(-c3ccc4nn(C)cc4n3)c(C)c(=O)c2c1 ; Cc1cc([C@@H](C)Nc2ccc(Cl)nc2-c2noc(=O)[nH]2)c2oc(-c3ccc4c(cnn4C)c3)c(C)c(=O)c2c1 ; Cc1cc([C@@H](C)Nc2cccc(F)c2-c2noc(=O)[nH]2)c2oc(-c3cncc(F)c3)c(C)c(=O)c2c1 ; Cc1cc([C@@H](C)Nc2ccc(F)c(F)c2-c2noc(=O)[nH]2)c2oc(-c3ccccn3)c(C)c(=O)c2c1] 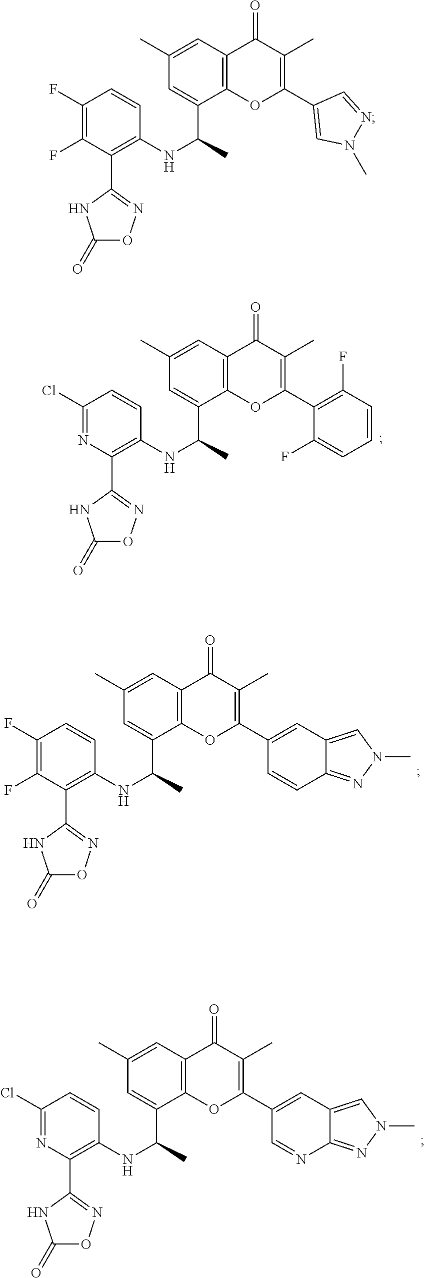 [SMILES: Cc1cc([C@@H](C)Nc2ccc(F)c(F)c2-c2noc(=O)[nH]2)c2oc(-c3cnn(C)c3)c(C)c(=O)c2c1 ; Cc1cc([C@@H](C)Nc2ccc(Cl)nc2-c2noc(=O)[nH]2)c2oc(-c3c(F)cccc3F)c(C)c(=O)c2c1 ; Cc1cc([C@@H](C)Nc2ccc(F)c(F)c2-c2noc(=O)[nH]2)c2oc(-c3ccc4nn(C)cc4c3)c(C)c(=O)c2c1 ; Cc1cc([C@@H](C)Nc2ccc(Cl)nc2-c2noc(=O)[nH]2)c2oc(-c3cnc4nn(C)cc4c3)c(C)c(=O)c2c1] 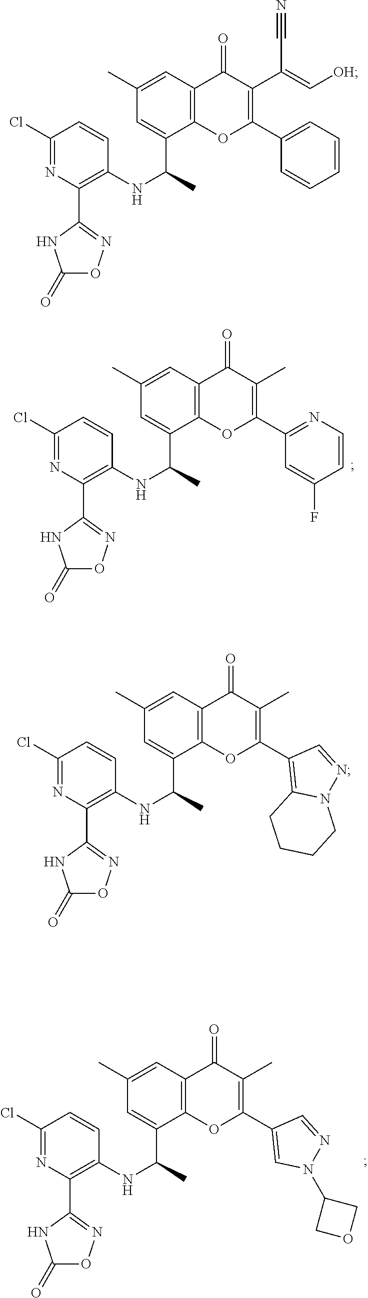 [SMILES: Cc1cc([C@@H](C)Nc2ccc(Cl)nc2-c2noc(=O)[nH]2)c2oc(-c3ccccc3)c(/C(C#N)=C/O)c(=O)c2c1 ; Cc1cc([C@@H](C)Nc2ccc(Cl)nc2-c2noc(=O)[nH]2)c2oc(-c3cc(F)ccn3)c(C)c(=O)c2c1 ; Cc1cc([C@@H](C)Nc2ccc(Cl)nc2-c2noc(=O)[nH]2)c2oc(-c3cnn4c3CCCC4)c(C)c(=O)c2c1 ; Cc1cc([C@@H](C)Nc2ccc(Cl)nc2-c2noc(=O)[nH]2)c2oc(-c3cnn(C4COC4)c3)c(C)c(=O)c2c1] 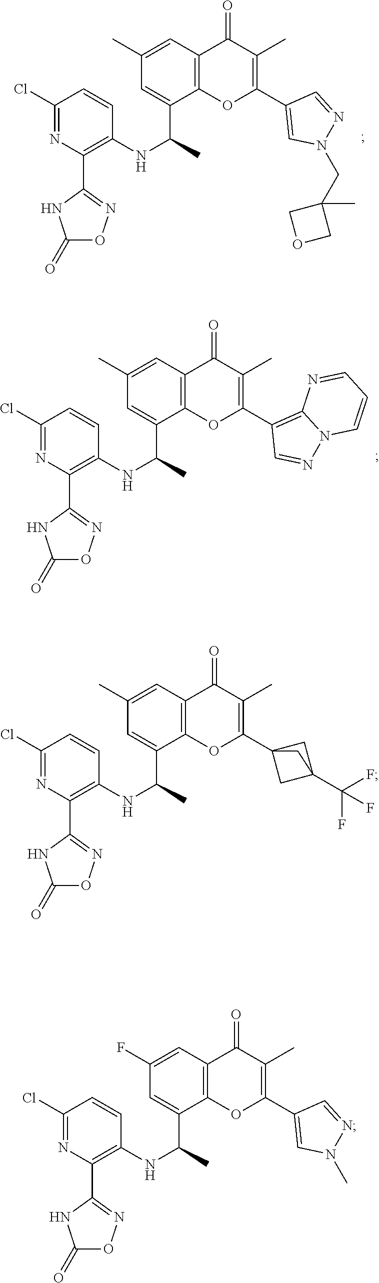 [SMILES: Cc1cc([C@@H](C)Nc2ccc(Cl)nc2-c2noc(=O)[nH]2)c2oc(-c3cnn(CC4(C)COC4)c3)c(C)c(=O)c2c1 ; Cc1cc([C@@H](C)Nc2ccc(Cl)nc2-c2noc(=O)[nH]2)c2oc(-c3cnn4cccnc34)c(C)c(=O)c2c1 ; Cc1cc([C@@H](C)Nc2ccc(Cl)nc2-c2noc(=O)[nH]2)c2oc(C34CC(C(F)(F)F)(C3)C4)c(C)c(=O)c2c1 ; Cc1c(-c2cnn(C)c2)oc2c([C@@H](C)Nc3ccc(Cl)nc3-c3noc(=O)[nH]3)cc(F)cc2c1=O] 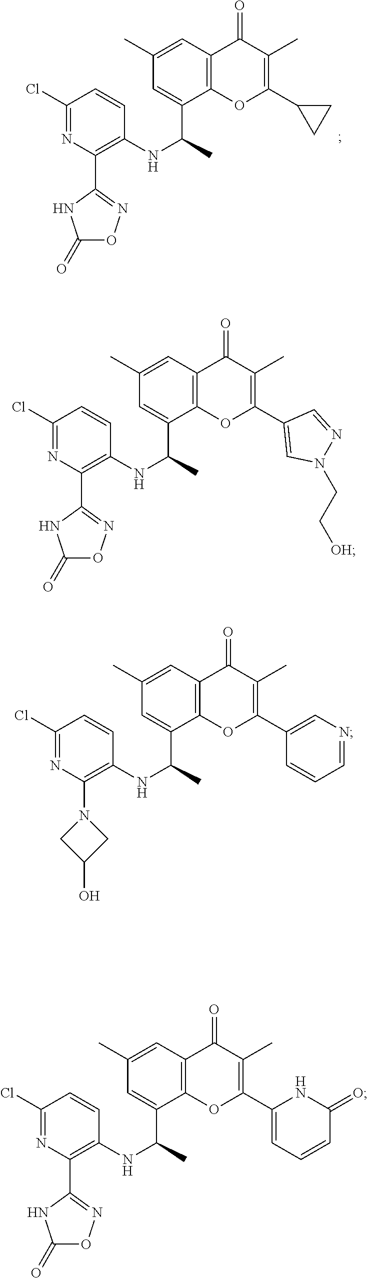 [SMILES: Cc1cc([C@@H](C)Nc2ccc(Cl)nc2-c2noc(=O)[nH]2)c2oc(C3CC3)c(C)c(=O)c2c1 ; Cc1cc([C@@H](C)Nc2ccc(Cl)nc2-c2noc(=O)[nH]2)c2oc(-c3cnn(CCO)c3)c(C)c(=O)c2c1 ; Cc1cc([C@@H](C)Nc2ccc(Cl)nc2N2CC(O)C2)c2oc(-c3cccnc3)c(C)c(=O)c2c1 ; Cc1cc([C@@H](C)Nc2ccc(Cl)nc2-c2noc(=O)[nH]2)c2oc(-c3cccc(=O)[nH]3)c(C)c(=O)c2c1] 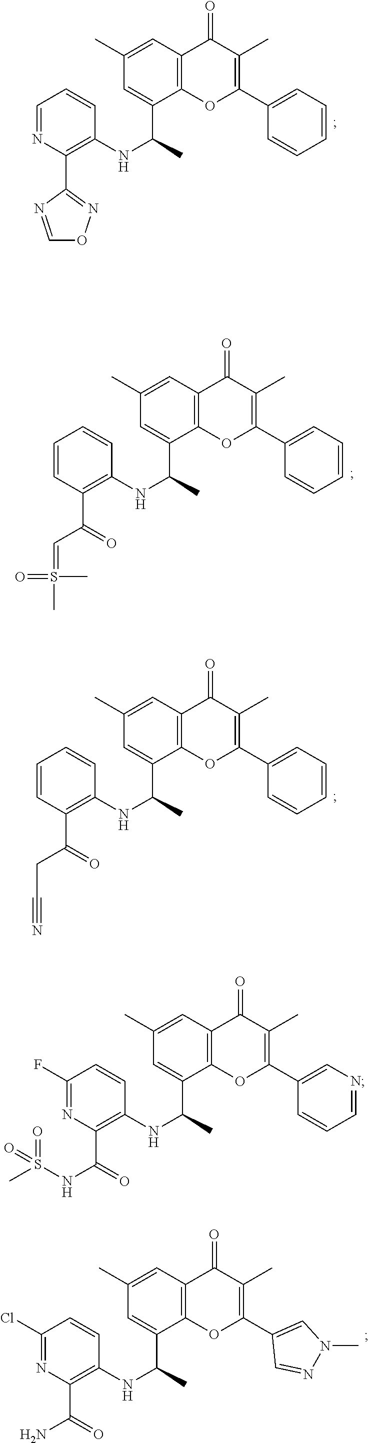 [SMILES: Cc1cc([C@@H](C)Nc2cccnc2-c2ncon2)c2oc(-c3ccccc3)c(C)c(=O)c2c1 ; Cc1cc([C@@H](C)Nc2ccccc2C(=O)C=S(C)(C)=O)c2oc(-c3ccccc3)c(C)c(=O)c2c1 ; Cc1cc([C@@H](C)Nc2ccccc2C(=O)CC#N)c2oc(-c3ccccc3)c(C)c(=O)c2c1 ; Cc1cc([C@@H](C)Nc2ccc(F)nc2C(=O)NS(C)(=O)=O)c2oc(-c3cccnc3)c(C)c(=O)c2c1 ; Cc1cc([C@@H](C)Nc2ccc(Cl)nc2C(N)=O)c2oc(-c3cnn(C)c3)c(C)c(=O)c2c1] 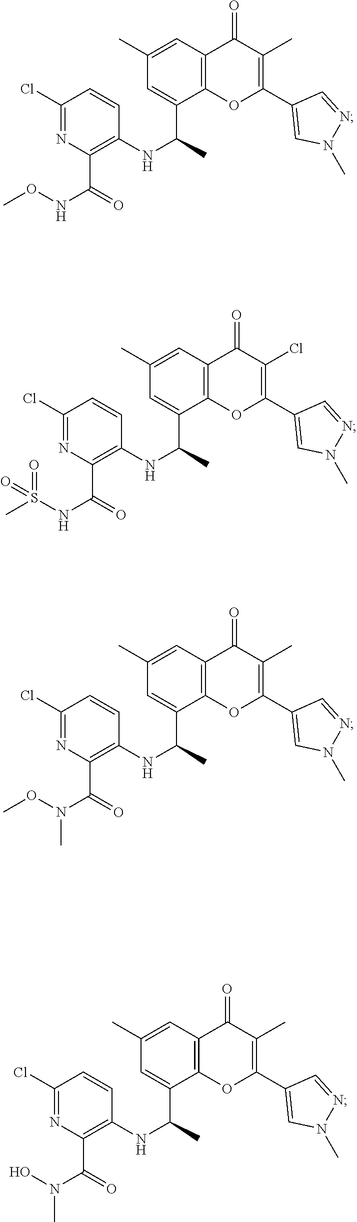 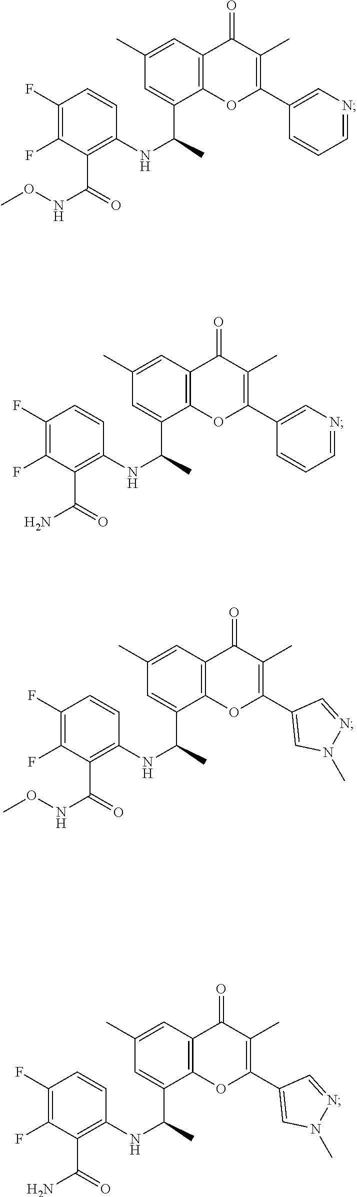 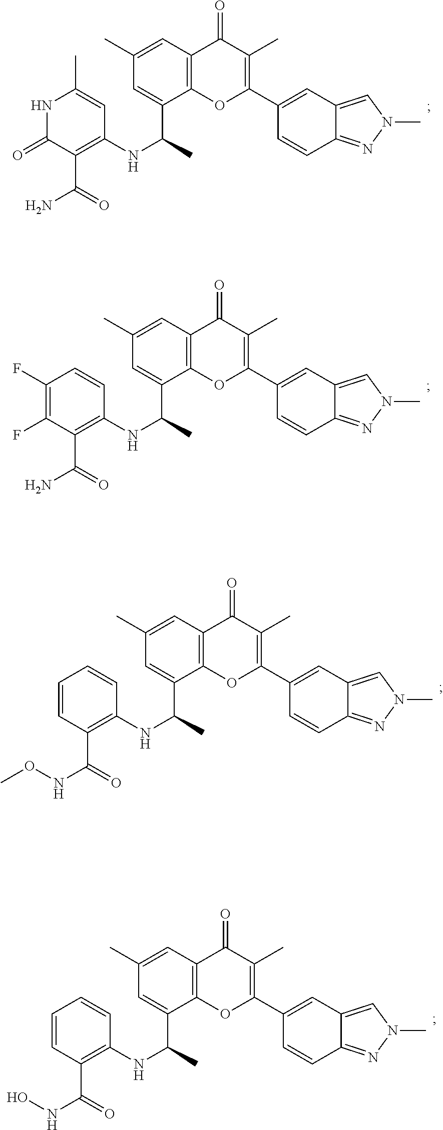 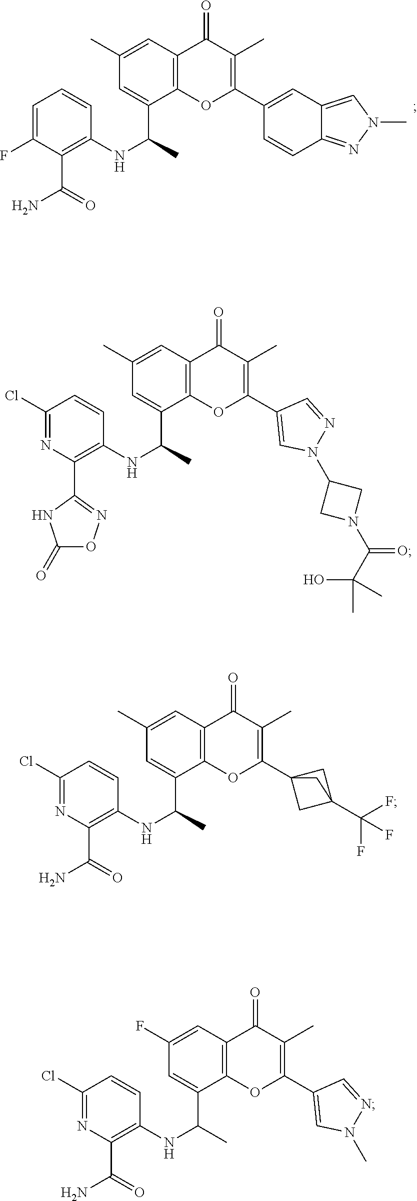 [SMILES: Cc1cc([C@@H](C)Nc2cccc(F)c2C(N)=O)c2oc(-c3ccc4nn(C)cc4c3)c(C)c(=O)c2c1 ; Cc1cc([C@@H](C)Nc2ccc(Cl)nc2-c2noc(=O)[nH]2)c2oc(-c3cnn(C4CN(C(=O)C(C)(C)O)C4)c3)c(C)c(=O)c2c1 ; Cc1cc([C@@H](C)Nc2ccc(Cl)nc2C(N)=O)c2oc(C34CC(C(F)(F)F)(C3)C4)c(C)c(=O)c2c1 ; Cc1c(-c2cnn(C)c2)oc2c(C(C)Nc3ccc(Cl)nc3C(N)=O)cc(F)cc2c1=O] 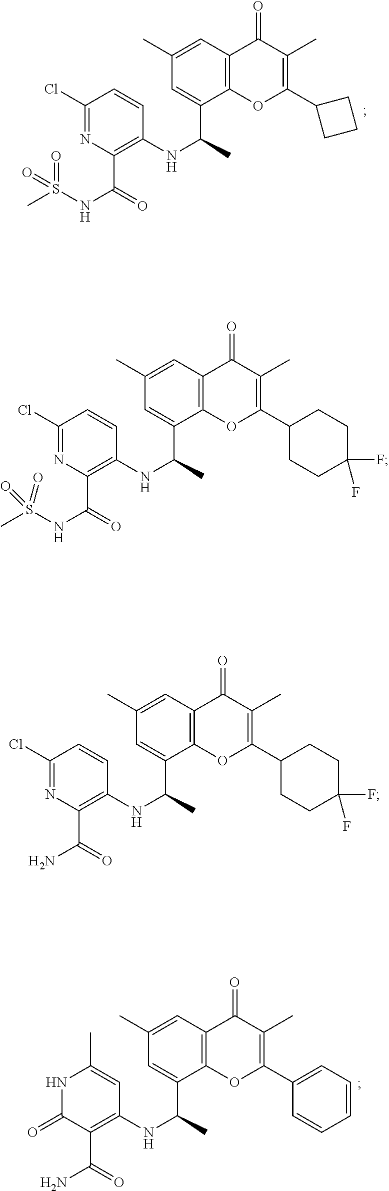 [SMILES: Cc1cc([C@@H](C)Nc2ccc(Cl)nc2C(=O)NS(C)(=O)=O)c2oc(C3CCC3)c(C)c(=O)c2c1 ; Cc1cc([C@@H](C)Nc2ccc(Cl)nc2C(=O)NS(C)(=O)=O)c2oc(C3CCC(F)(F)CC3)c(C)c(=O)c2c1 ; Cc1cc([C@@H](C)Nc2ccc(Cl)nc2C(N)=O)c2oc(C3CCC(F)(F)CC3)c(C)c(=O)c2c1 ; Cc1cc([C@@H](C)Nc2cc(C)[nH]c(=O)c2C(N)=O)c2oc(-c3ccccc3)c(C)c(=O)c2c1] 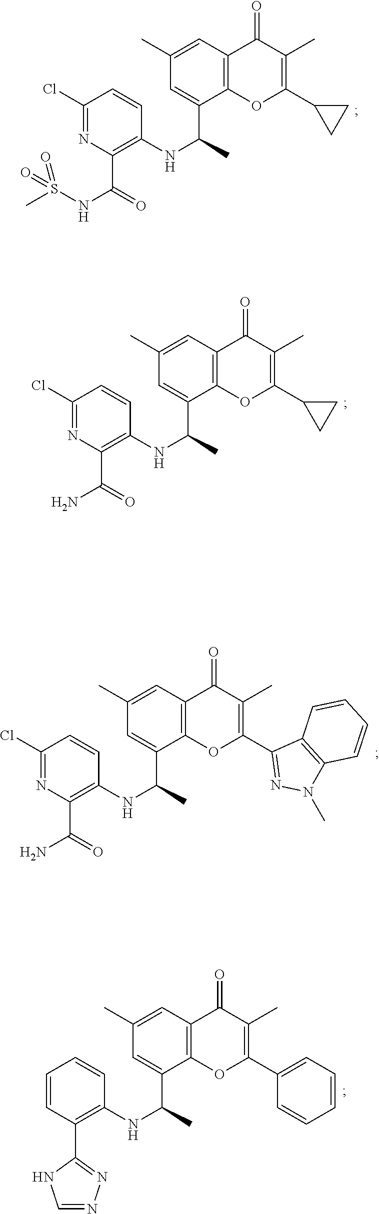 [SMILES: Cc1cc([C@@H](C)Nc2ccc(Cl)nc2C(=O)NS(C)(=O)=O)c2oc(C3CC3)c(C)c(=O)c2c1 ; Cc1cc([C@@H](C)Nc2ccc(Cl)nc2C(N)=O)c2oc(C3CC3)c(C)c(=O)c2c1 ; Cc1cc([C@@H](C)Nc2ccc(Cl)nc2C(N)=O)c2oc(-c3nn(C)c4ccccc34)c(C)c(=O)c2c1 ; Cc1cc([C@@H](C)Nc2ccccc2-c2nnc[nH]2)c2oc(-c3ccccc3)c(C)c(=O)c2c1] 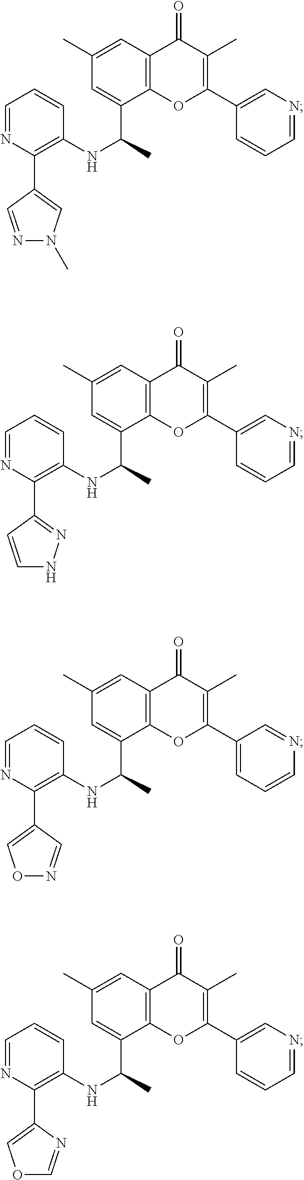 [SMILES: Cc1cc([C@@H](C)Nc2cccnc2-c2cnn(C)c2)c2oc(-c3cccnc3)c(C)c(=O)c2c1 ; Cc1cc([C@@H](C)Nc2cccnc2-c2cc[nH]n2)c2oc(-c3cccnc3)c(C)c(=O)c2c1 ; Cc1cc([C@@H](C)Nc2cccnc2-c2cnoc2)c2oc(-c3cccnc3)c(C)c(=O)c2c1 ; Cc1cc([C@@H](C)Nc2cccnc2-c2cocn2)c2oc(-c3cccnc3)c(C)c(=O)c2c1] 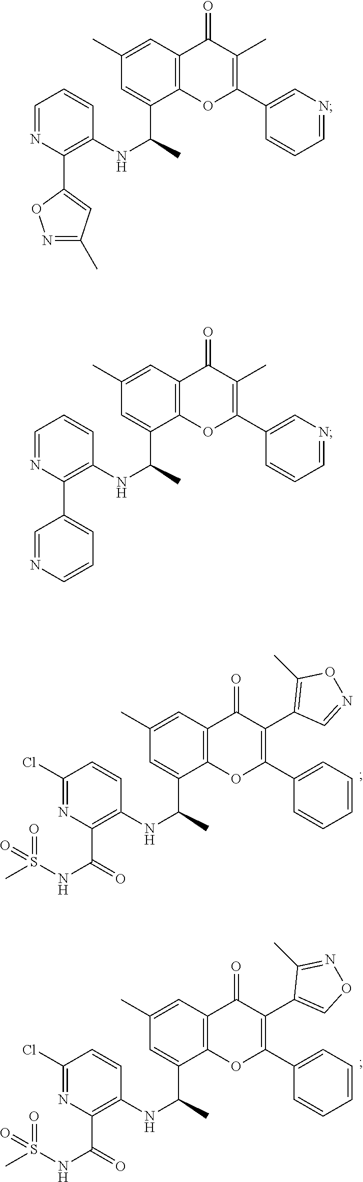 [SMILES: Cc1cc([C@@H](C)Nc2cccnc2-c2cc(C)no2)c2oc(-c3cccnc3)c(C)c(=O)c2c1 ; Cc1cc([C@@H](C)Nc2cccnc2-c2cccnc2)c2oc(-c3cccnc3)c(C)c(=O)c2c1 ; Cc1cc([C@@H](C)Nc2ccc(Cl)nc2C(=O)NS(C)(=O)=O)c2oc(-c3ccccc3)c(-c3cnoc3C)c(=O)c2c1 ; Cc1cc([C@@H](C)Nc2ccc(Cl)nc2C(=O)NS(C)(=O)=O)c2oc(-c3ccccc3)c(-c3conc3C)c(=O)c2c1] 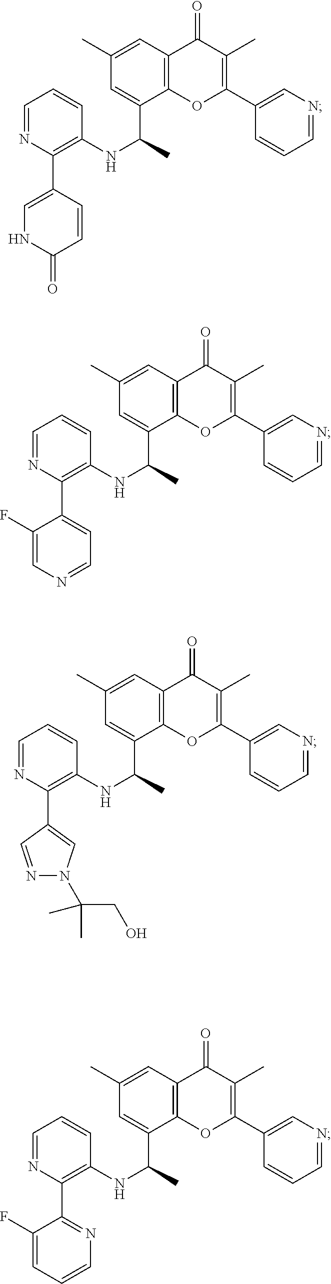 [SMILES: Cc1cc([C@@H](C)Nc2cccnc2-c2ccc(=O)[nH]c2)c2oc(-c3cccnc3)c(C)c(=O)c2c1 ; Cc1cc([C@@H](C)Nc2cccnc2-c2ccncc2F)c2oc(-c3cccnc3)c(C)c(=O)c2c1 ; Cc1cc([C@@H](C)Nc2cccnc2-c2cnn(C(C)(C)CO)c2)c2oc(-c3cccnc3)c(C)c(=O)c2c1 ; Cc1cc([C@@H](C)Nc2cccnc2-c2ncccc2F)c2oc(-c3cccnc3)c(C)c(=O)c2c1] 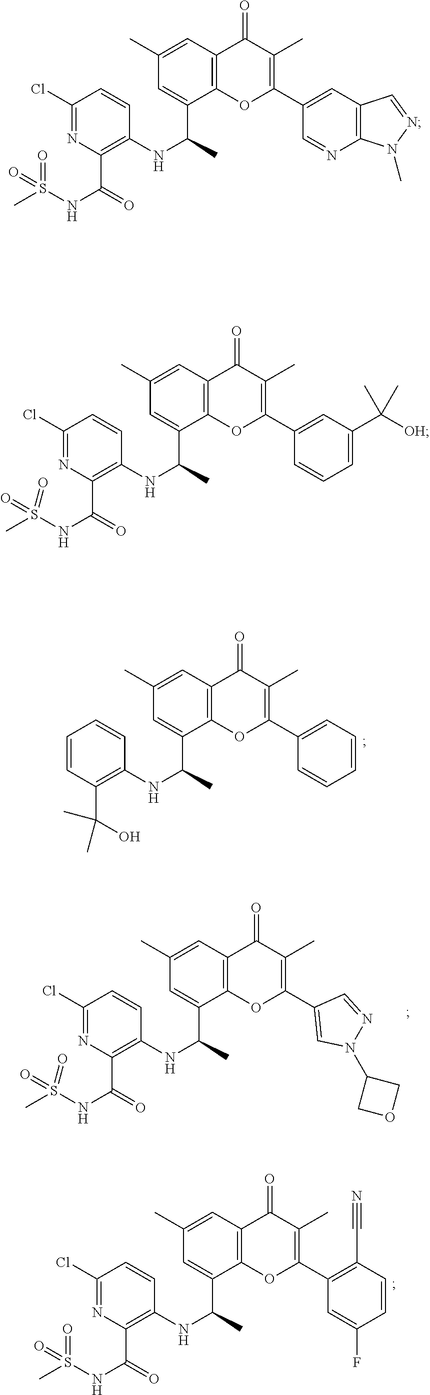 [SMILES: Cc1cc([C@@H](C)Nc2ccc(Cl)nc2C(=O)NS(C)(=O)=O)c2oc(-c3cnc4c(cnn4C)c3)c(C)c(=O)c2c1 ; Cc1cc([C@@H](C)Nc2ccc(Cl)nc2C(=O)NS(C)(=O)=O)c2oc(-c3cccc(C(C)(C)O)c3)c(C)c(=O)c2c1 ; Cc1cc([C@@H](C)Nc2ccccc2C(C)(C)O)c2oc(-c3ccccc3)c(C)c(=O)c2c1 ; Cc1cc([C@@H](C)Nc2ccc(Cl)nc2C(=O)NS(C)(=O)=O)c2oc(-c3cnn(C4COC4)c3)c(C)c(=O)c2c1 ; Cc1cc([C@@H](C)Nc2ccc(Cl)nc2C(=O)NS(C)(=O)=O)c2oc(-c3cc(F)ccc3C#N)c(C)c(=O)c2c1] 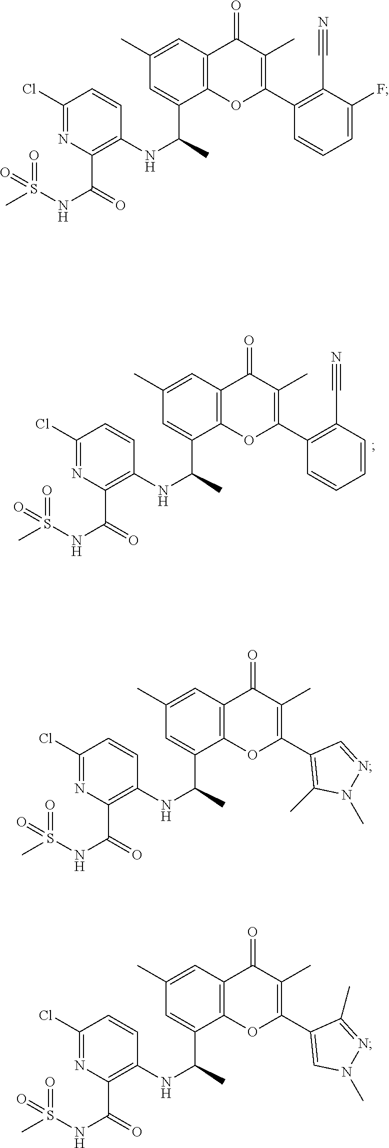 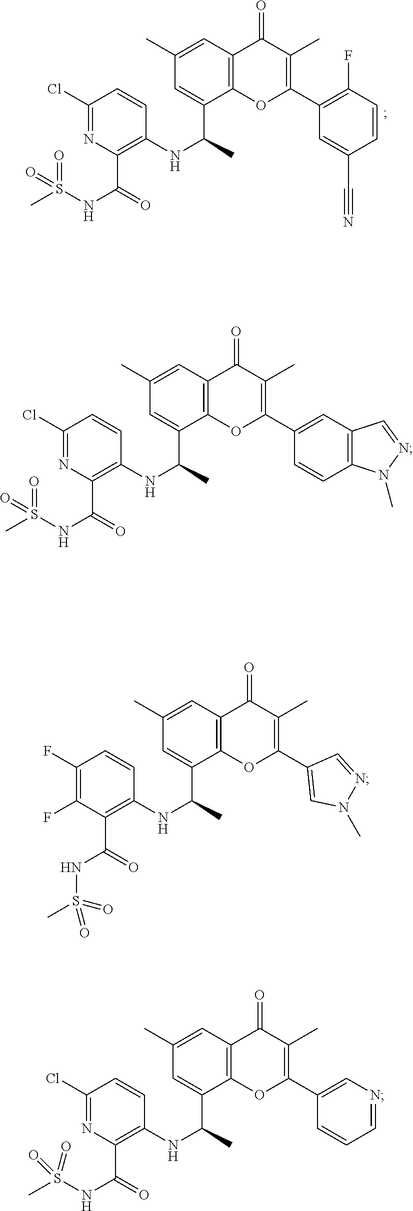 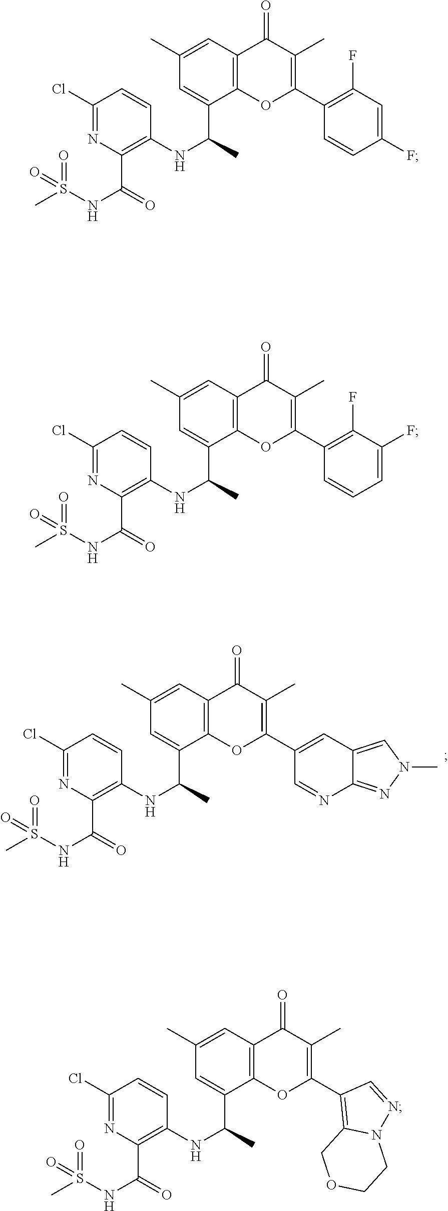 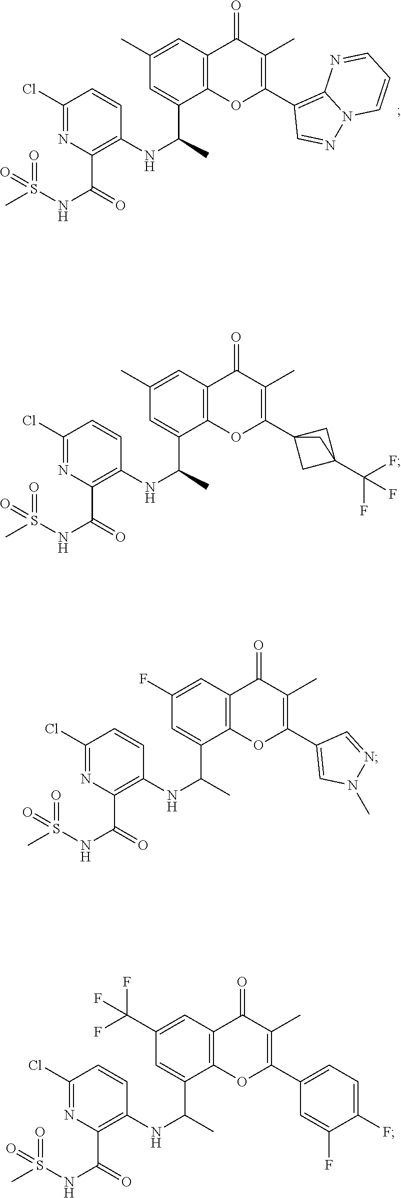 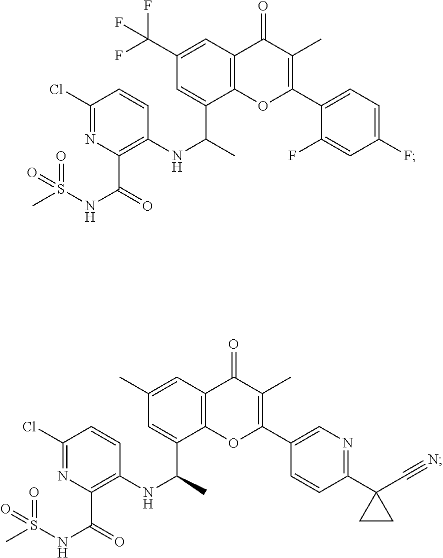 [SMILES: Cc1c(-c2ccc(F)cc2F)oc2c(C(C)Nc3ccc(Cl)nc3C(=O)NS(C)(=O)=O)cc(C(F)(F)F)cc2c1=O ; Cc1cc([C@@H](C)Nc2ccc(Cl)nc2C(=O)NS(C)(=O)=O)c2oc(-c3ccc(C4(C#N)CC4)nc3)c(C)c(=O)c2c1] 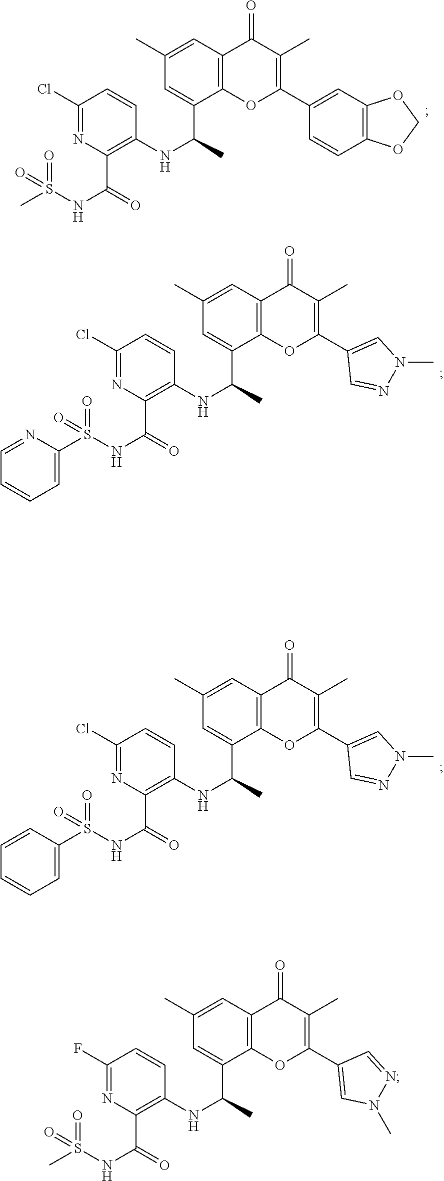 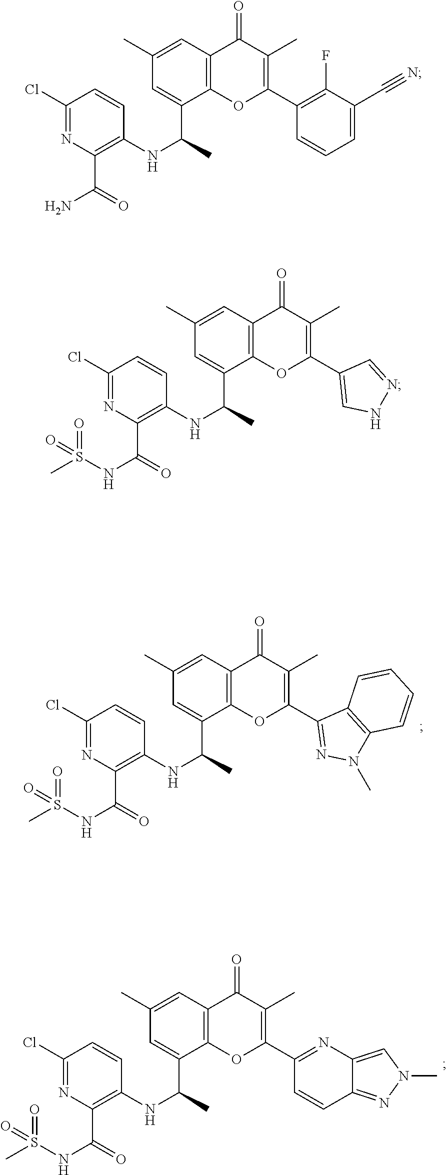 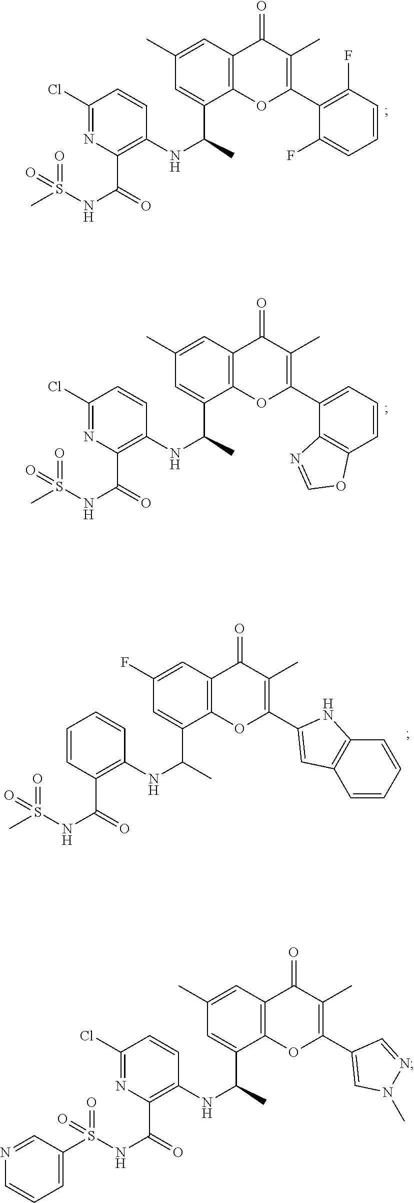 [SMILES: Cc1cc([C@@H](C)Nc2ccc(Cl)nc2C(=O)NS(C)(=O)=O)c2oc(-c3c(F)cccc3F)c(C)c(=O)c2c1 ; Cc1cc([C@@H](C)Nc2ccc(Cl)nc2C(=O)NS(C)(=O)=O)c2oc(-c3cccc4ocnc34)c(C)c(=O)c2c1 ; Cc1c(-c2cc3ccccc3[nH]2)oc2c(C(C)Nc3ccccc3C(=O)NS(C)(=O)=O)cc(F)cc2c1=O ; Cc1cc([C@@H](C)Nc2ccc(Cl)nc2C(=O)NS(=O)(=O)c2cccnc2)c2oc(-c3cnn(C)c3)c(C)c(=O)c2c1] 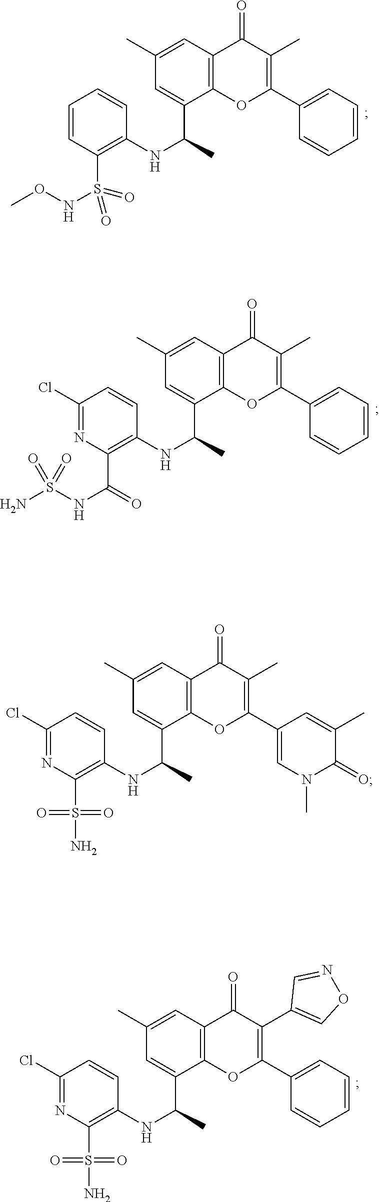 [SMILES: CONS(=O)(=O)c1ccccc1N[C@H](C)c1cc(C)cc2c(=O)c(C)c(-c3ccccc3)oc12 ; Cc1cc([C@@H](C)Nc2ccc(Cl)nc2C(=O)NS(N)(=O)=O)c2oc(-c3ccccc3)c(C)c(=O)c2c1 ; Cc1cc([C@@H](C)Nc2ccc(Cl)nc2S(N)(=O)=O)c2oc(-c3cc(C)c(=O)n(C)c3)c(C)c(=O)c2c1 ; Cc1cc([C@@H](C)Nc2ccc(Cl)nc2S(N)(=O)=O)c2oc(-c3ccccc3)c(-c3cnoc3)c(=O)c2c1] 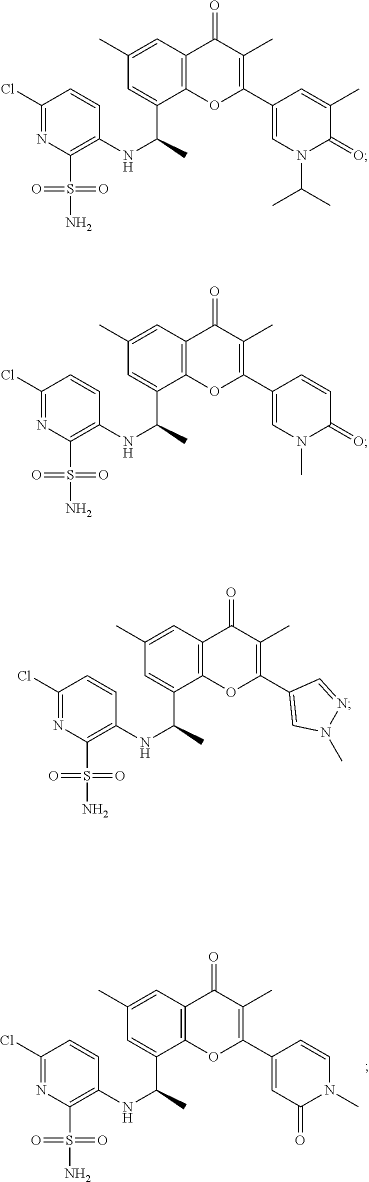 [SMILES: Cc1cc([C@@H](C)Nc2ccc(Cl)nc2S(N)(=O)=O)c2oc(-c3cc(C)c(=O)n(C(C)C)c3)c(C)c(=O)c2c1 ; Cc1cc([C@@H](C)Nc2ccc(Cl)nc2S(N)(=O)=O)c2oc(-c3ccc(=O)n(C)c3)c(C)c(=O)c2c1 ; Cc1cc([C@@H](C)Nc2ccc(Cl)nc2S(N)(=O)=O)c2oc(-c3cnn(C)c3)c(C)c(=O)c2c1 ; Cc1cc([C@@H](C)Nc2ccc(Cl)nc2S(N)(=O)=O)c2oc(-c3ccn(C)c(=O)c3)c(C)c(=O)c2c1] 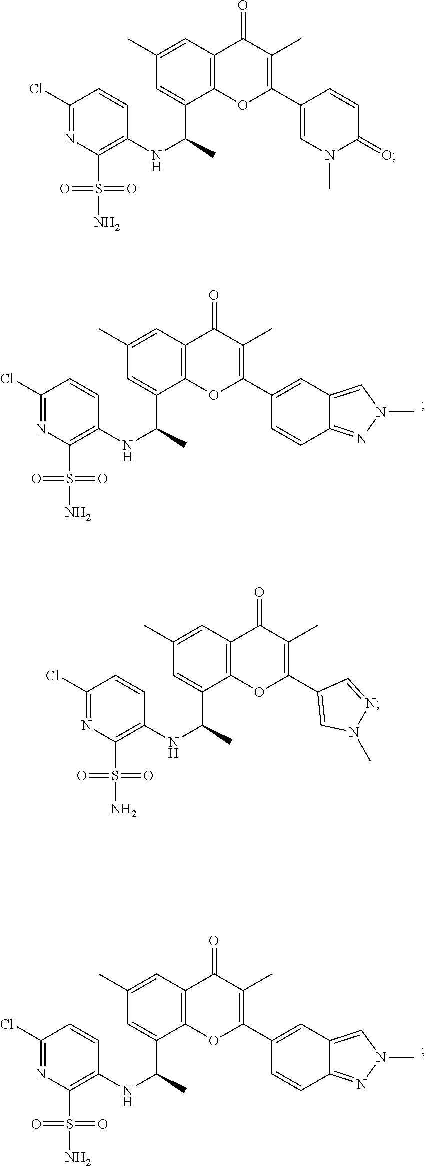 [SMILES: Cc1cc([C@@H](C)Nc2ccc(Cl)nc2S(N)(=O)=O)c2oc(-c3ccc(=O)n(C)c3)c(C)c(=O)c2c1 ; Cc1cc([C@@H](C)Nc2ccc(Cl)nc2S(N)(=O)=O)c2oc(-c3ccc4nn(C)cc4c3)c(C)c(=O)c2c1 ; Cc1cc([C@@H](C)Nc2ccc(Cl)nc2S(N)(=O)=O)c2oc(-c3cnn(C)c3)c(C)c(=O)c2c1 ; Cc1cc([C@@H](C)Nc2ccc(Cl)nc2S(N)(=O)=O)c2oc(-c3ccc4nn(C)cc4c3)c(C)c(=O)c2c1] 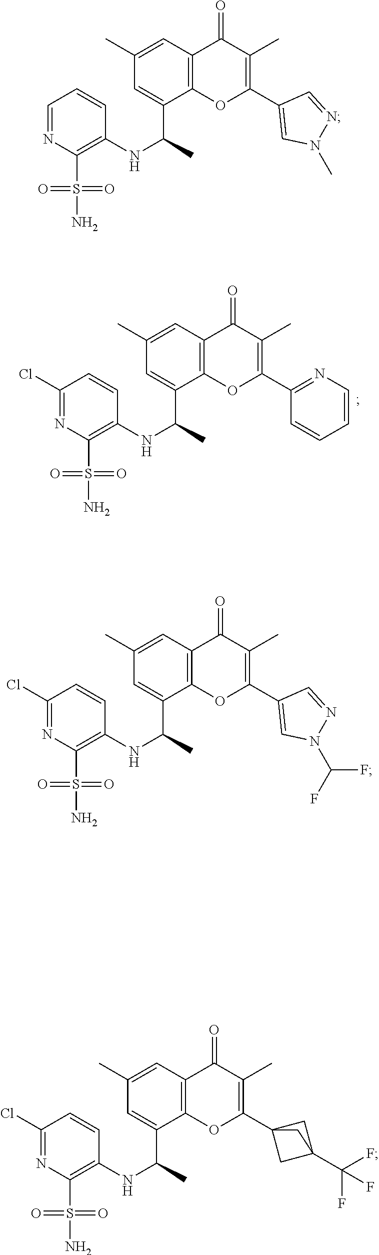 [SMILES: Cc1cc([C@@H](C)Nc2cccnc2S(N)(=O)=O)c2oc(-c3cnn(C)c3)c(C)c(=O)c2c1 ; Cc1cc([C@@H](C)Nc2ccc(Cl)nc2S(N)(=O)=O)c2oc(-c3ccccn3)c(C)c(=O)c2c1 ; Cc1cc([C@@H](C)Nc2ccc(Cl)nc2S(N)(=O)=O)c2oc(-c3cnn(C(F)F)c3)c(C)c(=O)c2c1 ; Cc1cc([C@@H](C)Nc2ccc(Cl)nc2S(N)(=O)=O)c2oc(C34CC(C(F)(F)F)(C3)C4)c(C)c(=O)c2c1] 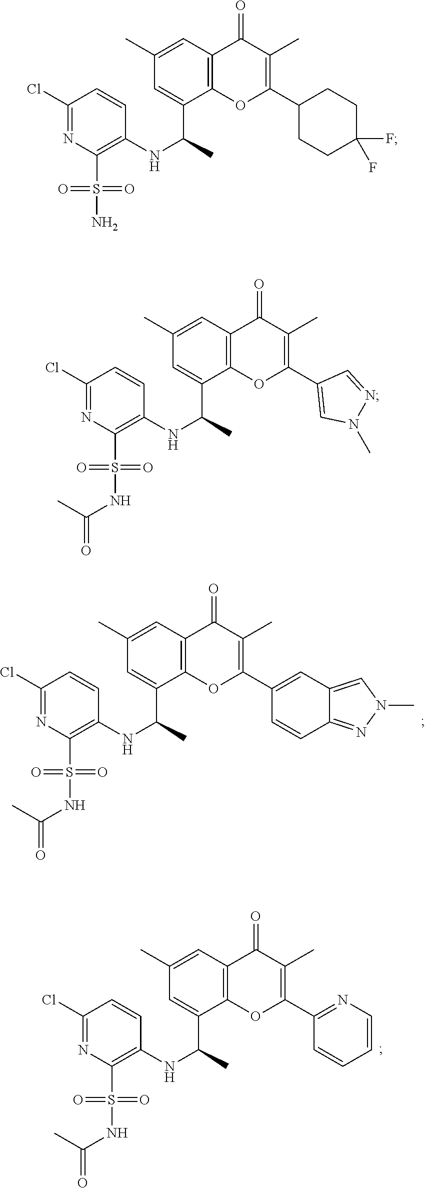 [SMILES: Cc1cc([C@@H](C)Nc2ccc(Cl)nc2S(N)(=O)=O)c2oc(C3CCC(F)(F)CC3)c(C)c(=O)c2c1 ; CC(=O)NS(=O)(=O)c1nc(Cl)ccc1N[C@H](C)c1cc(C)cc2c(=O)c(C)c(-c3cnn(C)c3)oc12 ; CC(=O)NS(=O)(=O)c1nc(Cl)ccc1N[C@H](C)c1cc(C)cc2c(=O)c(C)c(-c3ccc4nn(C)cc4c3)oc12 ; CC(=O)NS(=O)(=O)c1nc(Cl)ccc1N[C@H](C)c1cc(C)cc2c(=O)c(C)c(-c3ccccn3)oc12] 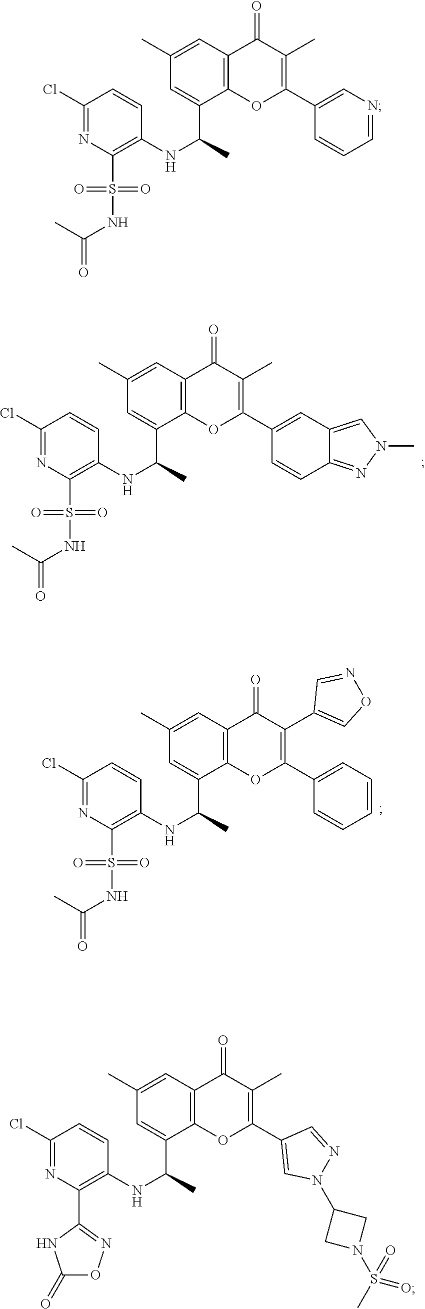 [SMILES: CC(=O)NS(=O)(=O)c1nc(Cl)ccc1N[C@H](C)c1cc(C)cc2c(=O)c(C)c(-c3cccnc3)oc12 ; CC(=O)NS(=O)(=O)c1nc(Cl)ccc1N[C@H](C)c1cc(C)cc2c(=O)c(C)c(-c3ccc4nn(C)cc4c3)oc12 ; CC(=O)NS(=O)(=O)c1nc(Cl)ccc1N[C@H](C)c1cc(C)cc2c(=O)c(-c3cnoc3)c(-c3ccccc3)oc12 ; Cc1cc([C@@H](C)Nc2ccc(Cl)nc2-c2noc(=O)[nH]2)c2oc(-c3cnn(C4CN(S(C)(=O)=O)C4)c3)c(C)c(=O)c2c1] 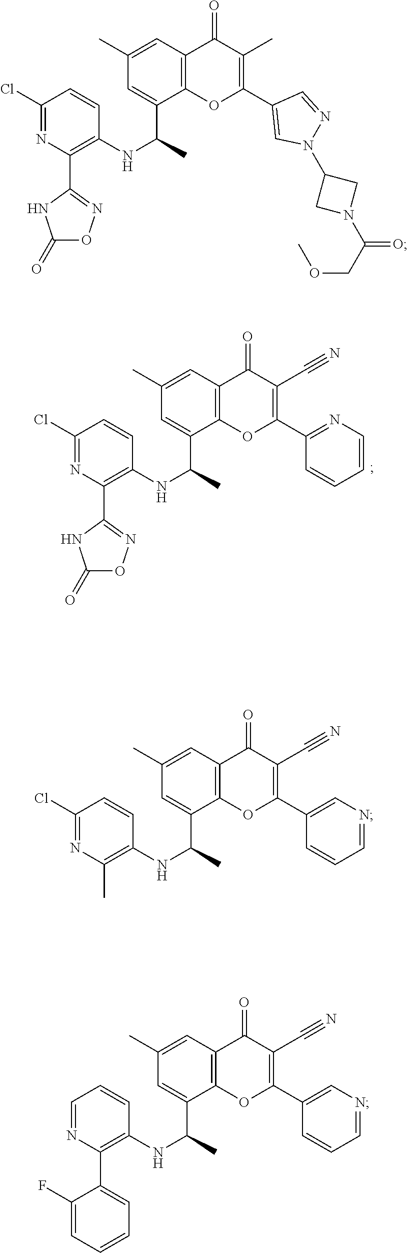 [SMILES: COCC(=O)N1CC(n2cc(-c3oc4c([C@@H](C)Nc5ccc(Cl)nc5-c5noc(=O)[nH]5)cc(C)cc4c(=O)c3C)cn2)C1 ; Cc1cc([C@@H](C)Nc2ccc(Cl)nc2-c2noc(=O)[nH]2)c2oc(-c3ccccn3)c(C#N)c(=O)c2c1 ; Cc1cc([C@@H](C)Nc2ccc(Cl)nc2C)c2oc(-c3cccnc3)c(C#N)c(=O)c2c1 ; Cc1cc([C@@H](C)Nc2cccnc2-c2ccccc2F)c2oc(-c3cccnc3)c(C#N)c(=O)c2c1] 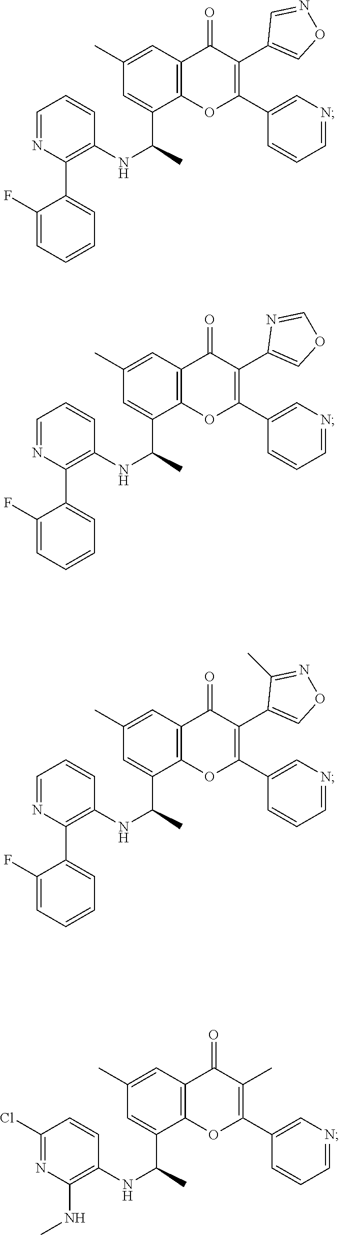 [SMILES: Cc1cc([C@@H](C)Nc2cccnc2-c2ccccc2F)c2oc(-c3cccnc3)c(-c3cnoc3)c(=O)c2c1 ; Cc1cc([C@@H](C)Nc2cccnc2-c2ccccc2F)c2oc(-c3cccnc3)c(-c3cocn3)c(=O)c2c1 ; Cc1cc([C@@H](C)Nc2cccnc2-c2ccccc2F)c2oc(-c3cccnc3)c(-c3conc3C)c(=O)c2c1 ; CNc1nc(Cl)ccc1N[C@H](C)c1cc(C)cc2c(=O)c(C)c(-c3cccnc3)oc12] 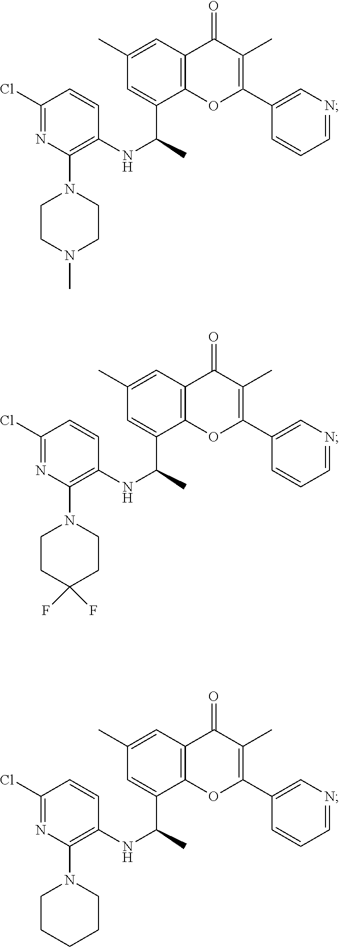 [SMILES: Cc1cc([C@@H](C)Nc2ccc(Cl)nc2N2CCN(C)CC2)c2oc(-c3cccnc3)c(C)c(=O)c2c1 ; Cc1cc([C@@H](C)Nc2ccc(Cl)nc2N2CCC(F)(F)CC2)c2oc(-c3cccnc3)c(C)c(=O)c2c1 ; Cc1cc([C@@H](C)Nc2ccc(Cl)nc2N2CCCCC2)c2oc(-c3cccnc3)c(C)c(=O)c2c1] 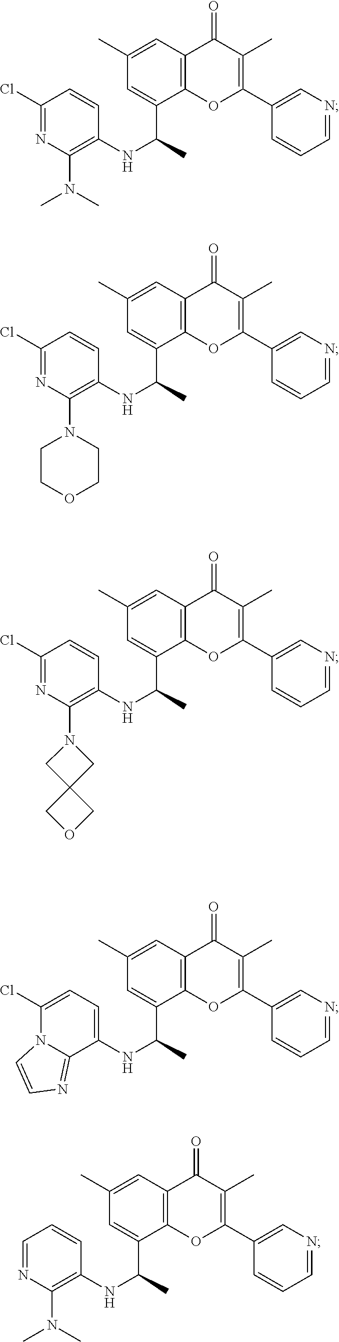 [SMILES: Cc1cc([C@@H](C)Nc2ccc(Cl)nc2N(C)C)c2oc(-c3cccnc3)c(C)c(=O)c2c1 ; Cc1cc([C@@H](C)Nc2ccc(Cl)nc2N2CCOCC2)c2oc(-c3cccnc3)c(C)c(=O)c2c1 ; Cc1cc([C@@H](C)Nc2ccc(Cl)nc2N2CC3(COC3)C2)c2oc(-c3cccnc3)c(C)c(=O)c2c1 ; Cc1cc([C@@H](C)Nc2ccc(Cl)n3ccnc23)c2oc(-c3cccnc3)c(C)c(=O)c2c1 ; Cc1cc([C@@H](C)Nc2cccnc2N(C)C)c2oc(-c3cccnc3)c(C)c(=O)c2c1] 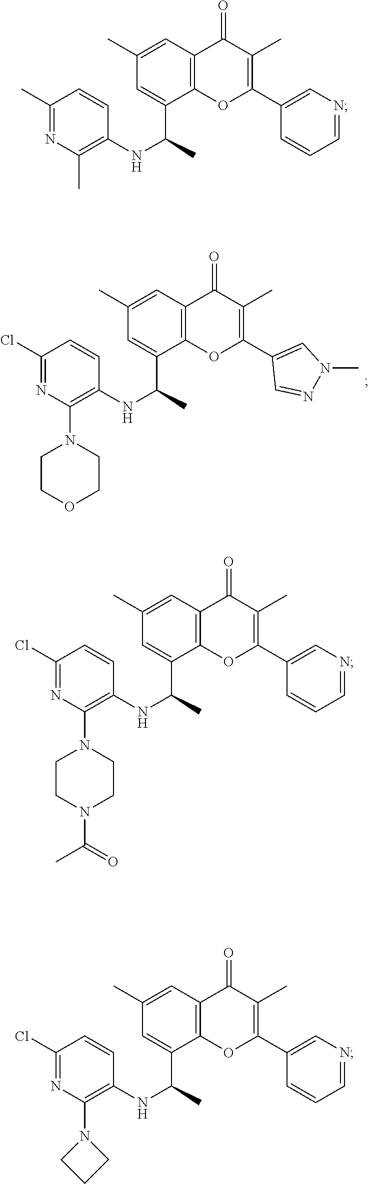 [SMILES: Cc1cc([C@@H](C)Nc2ccc(C)nc2C)c2oc(-c3cccnc3)c(C)c(=O)c2c1 ; Cc1cc([C@@H](C)Nc2ccc(Cl)nc2N2CCOCC2)c2oc(-c3cnn(C)c3)c(C)c(=O)c2c1 ; CC(=O)N1CCN(c2nc(Cl)ccc2N[C@H](C)c2cc(C)cc3c(=O)c(C)c(-c4cccnc4)oc23)CC1 ; Cc1cc([C@@H](C)Nc2ccc(Cl)nc2N2CCC2)c2oc(-c3cccnc3)c(C)c(=O)c2c1] 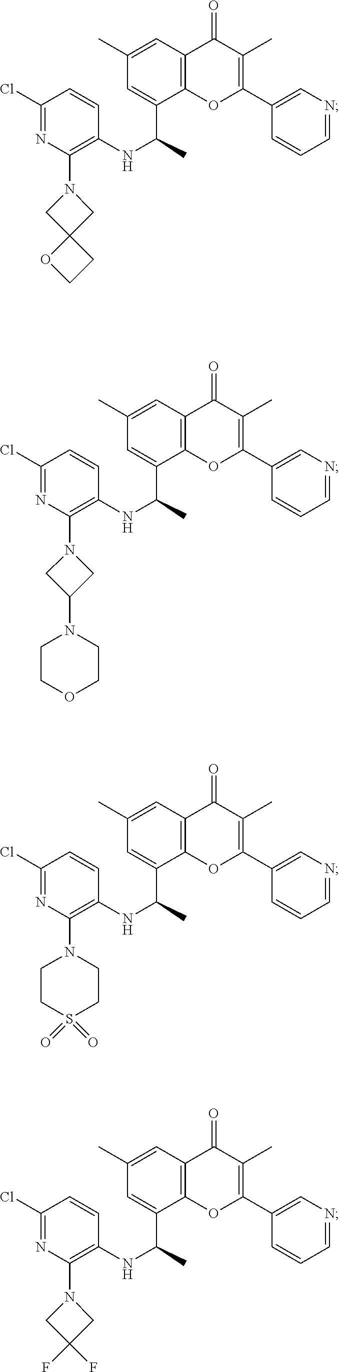 [SMILES: Cc1cc([C@@H](C)Nc2ccc(Cl)nc2N2CC3(CCO3)C2)c2oc(-c3cccnc3)c(C)c(=O)c2c1 ; Cc1cc([C@@H](C)Nc2ccc(Cl)nc2N2CC(N3CCOCC3)C2)c2oc(-c3cccnc3)c(C)c(=O)c2c1 ; Cc1cc([C@@H](C)Nc2ccc(Cl)nc2N2CCS(=O)(=O)CC2)c2oc(-c3cccnc3)c(C)c(=O)c2c1 ; Cc1cc([C@@H](C)Nc2ccc(Cl)nc2N2CC(F)(F)C2)c2oc(-c3cccnc3)c(C)c(=O)c2c1] 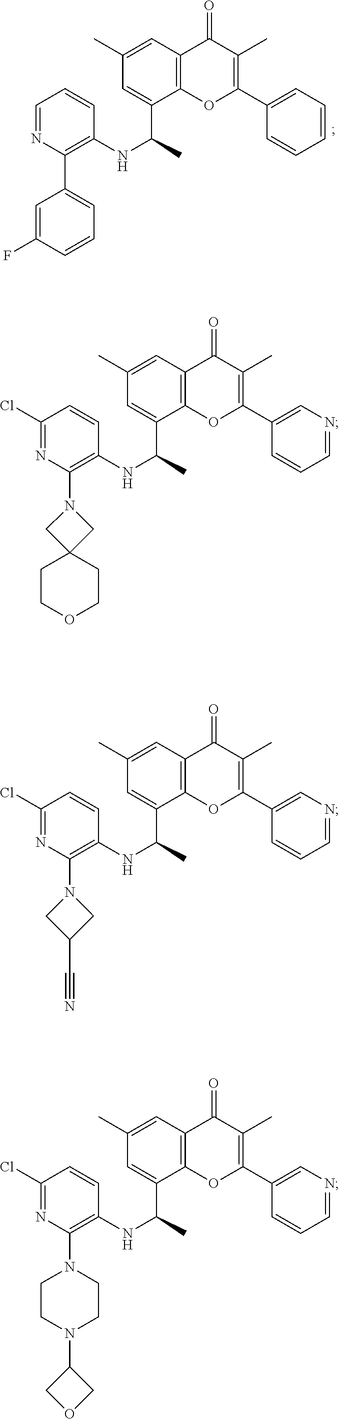 [SMILES: Cc1cc([C@@H](C)Nc2cccnc2-c2cccc(F)c2)c2oc(-c3ccccc3)c(C)c(=O)c2c1 ; Cc1cc([C@@H](C)Nc2ccc(Cl)nc2N2CC3(CCOCC3)C2)c2oc(-c3cccnc3)c(C)c(=O)c2c1 ; Cc1cc([C@@H](C)Nc2ccc(Cl)nc2N2CC(C#N)C2)c2oc(-c3cccnc3)c(C)c(=O)c2c1 ; Cc1cc([C@@H](C)Nc2ccc(Cl)nc2N2CCN(C3COC3)CC2)c2oc(-c3cccnc3)c(C)c(=O)c2c1] 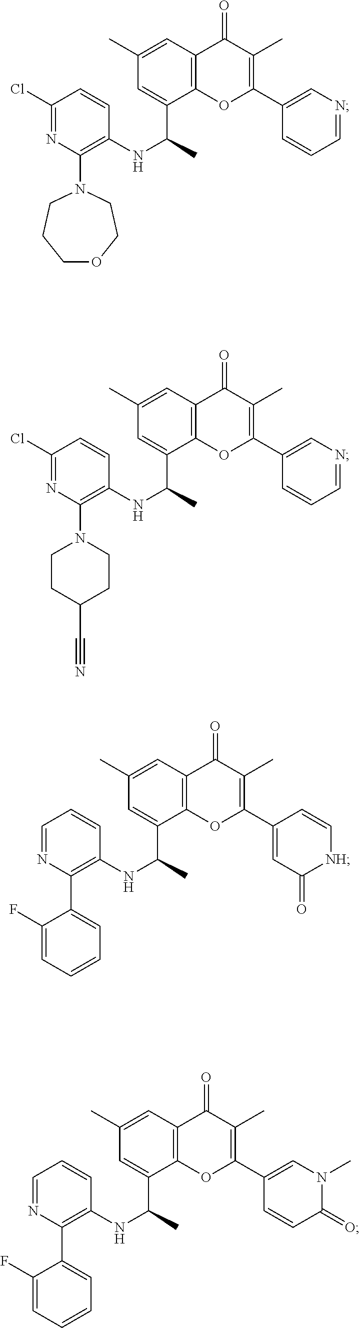 [SMILES: Cc1cc([C@@H](C)Nc2ccc(Cl)nc2N2CCCOCC2)c2oc(-c3cccnc3)c(C)c(=O)c2c1 ; Cc1cc([C@@H](C)Nc2ccc(Cl)nc2N2CCC(C#N)CC2)c2oc(-c3cccnc3)c(C)c(=O)c2c1 ; Cc1cc([C@@H](C)Nc2cccnc2-c2ccccc2F)c2oc(-c3cc[nH]c(=O)c3)c(C)c(=O)c2c1 ; Cc1cc([C@@H](C)Nc2cccnc2-c2ccccc2F)c2oc(-c3ccc(=O)n(C)c3)c(C)c(=O)c2c1] 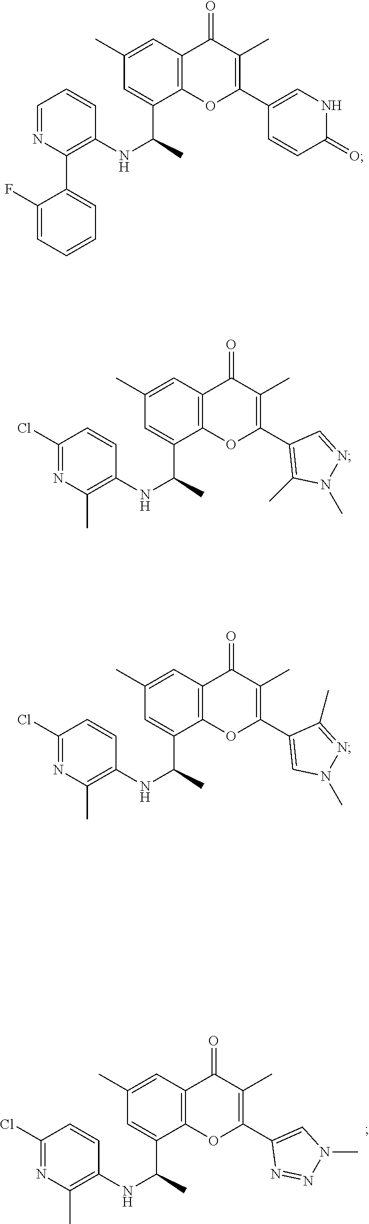 [SMILES: Cc1cc([C@@H](C)Nc2cccnc2-c2ccccc2F)c2oc(-c3ccc(=O)[nH]c3)c(C)c(=O)c2c1 ; Cc1cc([C@@H](C)Nc2ccc(Cl)nc2C)c2oc(-c3cnn(C)c3C)c(C)c(=O)c2c1 ; Cc1cc([C@@H](C)Nc2ccc(Cl)nc2C)c2oc(-c3cn(C)nc3C)c(C)c(=O)c2c1 ; Cc1cc([C@@H](C)Nc2ccc(Cl)nc2C)c2oc(-c3cn(C)nn3)c(C)c(=O)c2c1] 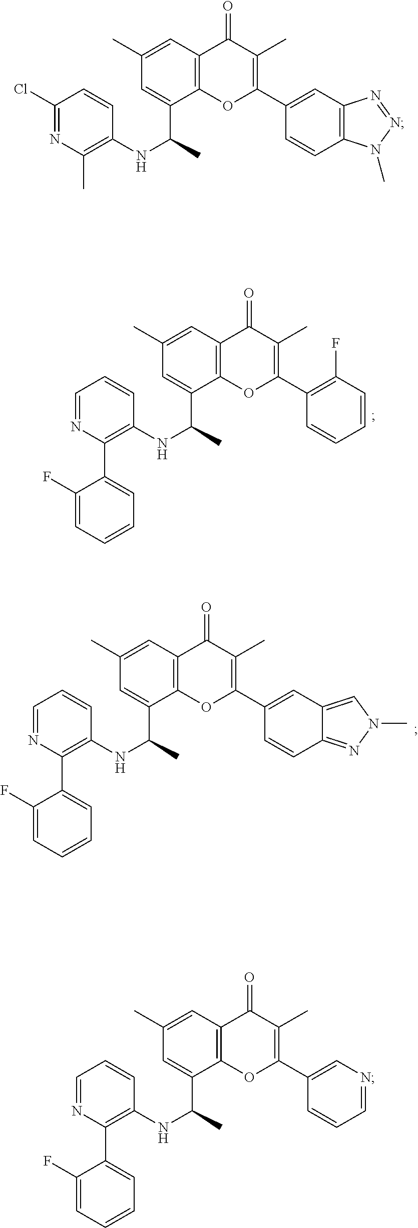 [SMILES: Cc1cc([C@@H](C)Nc2ccc(Cl)nc2C)c2oc(-c3ccc4c(c3)nnn4C)c(C)c(=O)c2c1 ; Cc1cc([C@@H](C)Nc2cccnc2-c2ccccc2F)c2oc(-c3ccccc3F)c(C)c(=O)c2c1 ; Cc1cc([C@@H](C)Nc2cccnc2-c2ccccc2F)c2oc(-c3ccc4nn(C)cc4c3)c(C)c(=O)c2c1 ; Cc1cc([C@@H](C)Nc2cccnc2-c2ccccc2F)c2oc(-c3cccnc3)c(C)c(=O)c2c1] 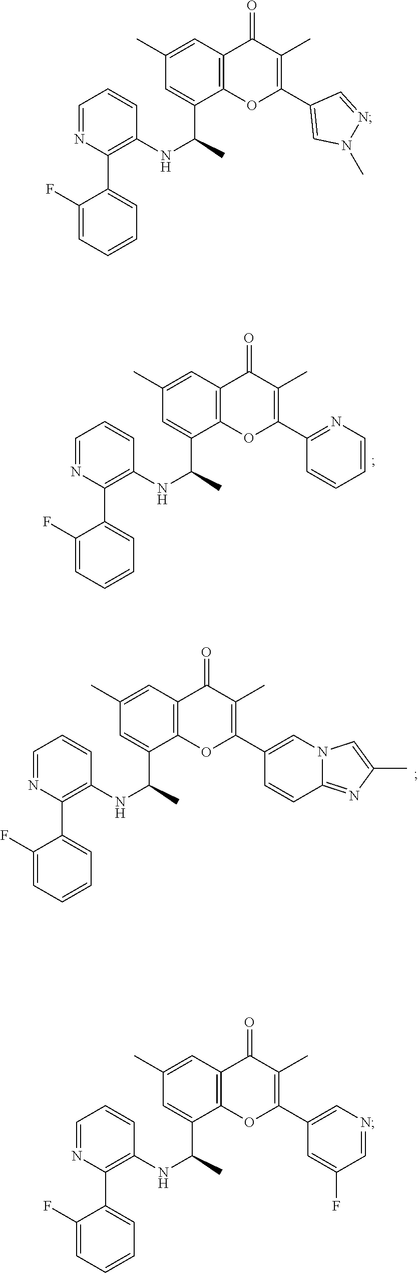 [SMILES: Cc1cc([C@@H](C)Nc2cccnc2-c2ccccc2F)c2oc(-c3cnn(C)c3)c(C)c(=O)c2c1 ; Cc1cc([C@@H](C)Nc2cccnc2-c2ccccc2F)c2oc(-c3ccccn3)c(C)c(=O)c2c1 ; Cc1cc([C@@H](C)Nc2cccnc2-c2ccccc2F)c2oc(-c3ccc4nc(C)cn4c3)c(C)c(=O)c2c1 ; Cc1cc([C@@H](C)Nc2cccnc2-c2ccccc2F)c2oc(-c3cncc(F)c3)c(C)c(=O)c2c1] 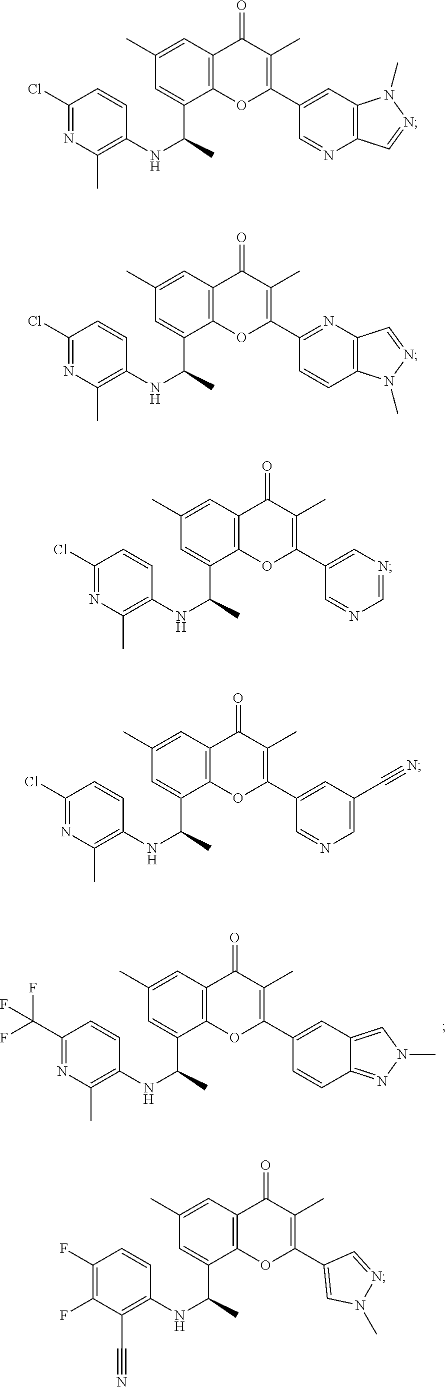 [SMILES: Cc1cc([C@@H](C)Nc2ccc(Cl)nc2C)c2oc(-c3cnc4cnn(C)c4c3)c(C)c(=O)c2c1 ; Cc1cc([C@@H](C)Nc2ccc(Cl)nc2C)c2oc(-c3ccc4c(cnn4C)n3)c(C)c(=O)c2c1 ; Cc1cc([C@@H](C)Nc2ccc(Cl)nc2C)c2oc(-c3cncnc3)c(C)c(=O)c2c1 ; Cc1cc([C@@H](C)Nc2ccc(Cl)nc2C)c2oc(-c3cncc(C#N)c3)c(C)c(=O)c2c1 ; Cc1cc([C@@H](C)Nc2ccc(C(F)(F)F)nc2C)c2oc(-c3ccc4nn(C)cc4c3)c(C)c(=O)c2c1 ; Cc1cc([C@@H](C)Nc2ccc(F)c(F)c2C#N)c2oc(-c3cnn(C)c3)c(C)c(=O)c2c1] 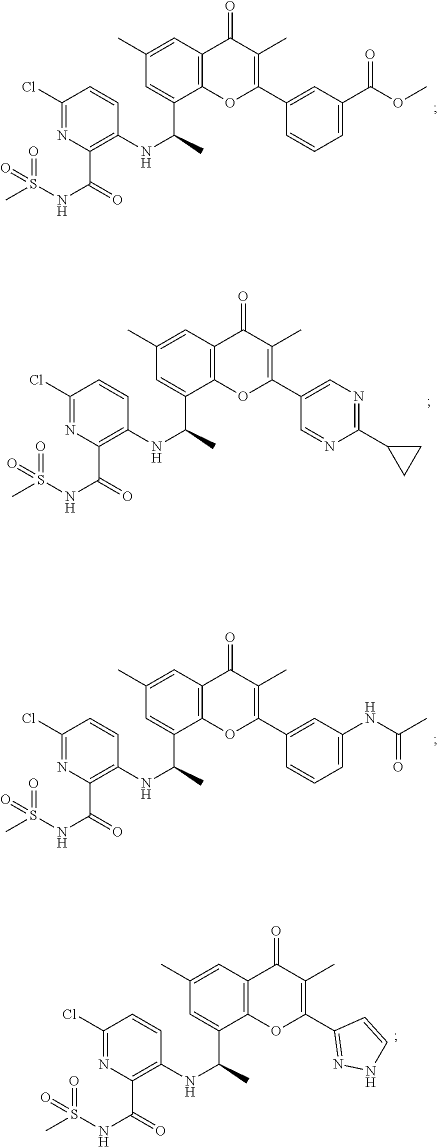 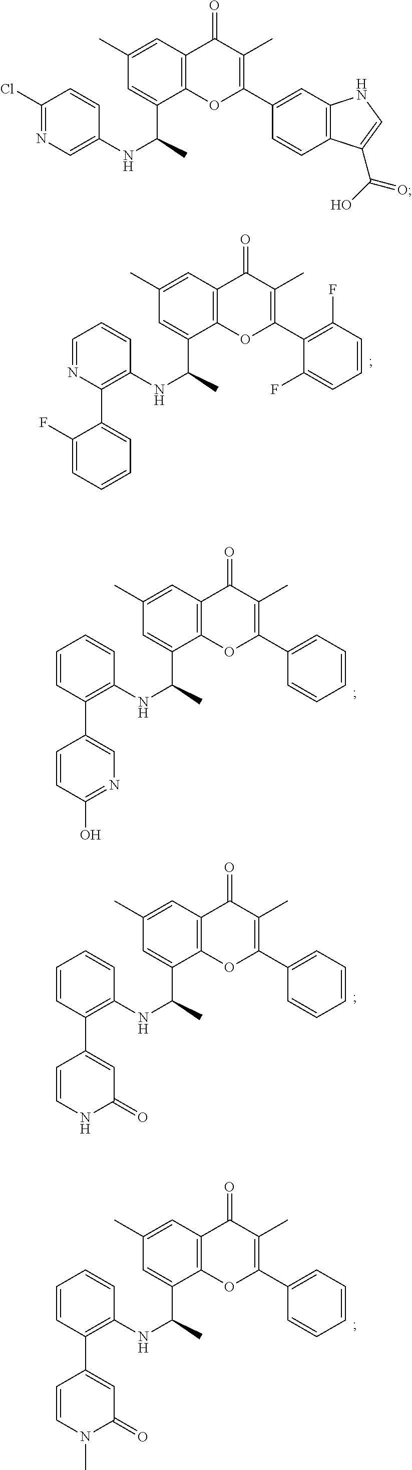 [SMILES: Cc1cc([C@@H](C)Nc2ccc(Cl)nc2)c2oc(-c3ccc4c(C(=O)O)c[nH]c4c3)c(C)c(=O)c2c1 ; Cc1cc([C@@H](C)Nc2cccnc2-c2ccccc2F)c2oc(-c3c(F)cccc3F)c(C)c(=O)c2c1 ; Cc1cc([C@@H](C)Nc2ccccc2-c2ccc(O)nc2)c2oc(-c3ccccc3)c(C)c(=O)c2c1 ; Cc1cc([C@@H](C)Nc2ccccc2-c2cc[nH]c(=O)c2)c2oc(-c3ccccc3)c(C)c(=O)c2c1 ; Cc1cc([C@@H](C)Nc2ccccc2-c2ccn(C)c(=O)c2)c2oc(-c3ccccc3)c(C)c(=O)c2c1] 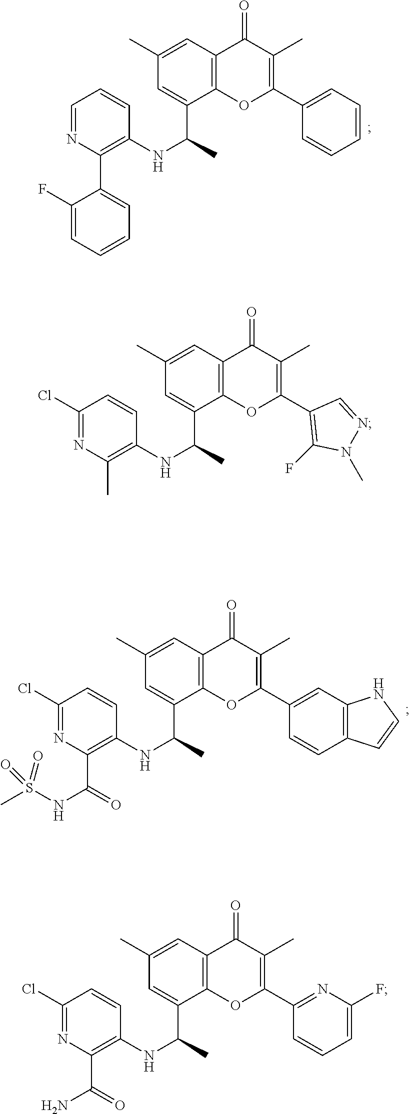 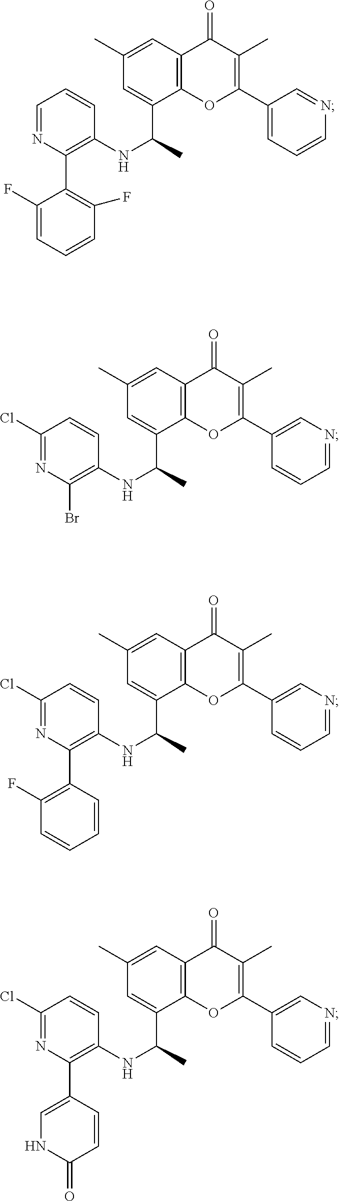 [SMILES: Cc1cc([C@@H](C)Nc2cccnc2-c2c(F)cccc2F)c2oc(-c3cccnc3)c(C)c(=O)c2c1 ; Cc1cc([C@@H](C)Nc2ccc(Cl)nc2Br)c2oc(-c3cccnc3)c(C)c(=O)c2c1 ; Cc1cc([C@@H](C)Nc2ccc(Cl)nc2-c2ccccc2F)c2oc(-c3cccnc3)c(C)c(=O)c2c1 ; Cc1cc([C@@H](C)Nc2ccc(Cl)nc2-c2ccc(=O)[nH]c2)c2oc(-c3cccnc3)c(C)c(=O)c2c1] 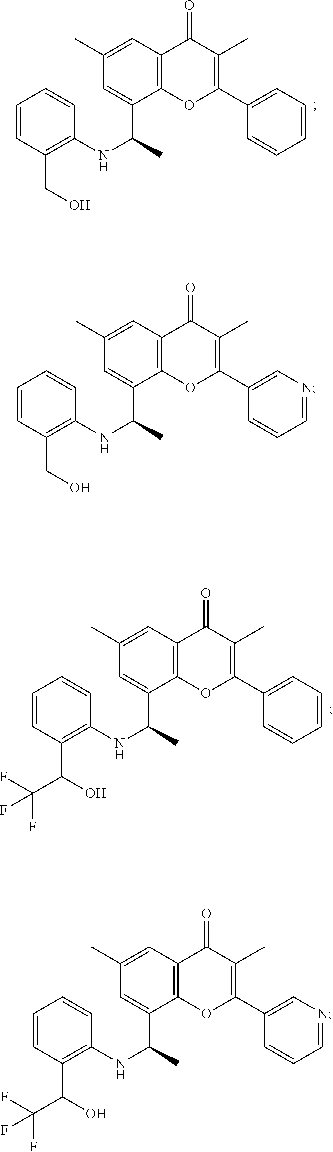 [SMILES: Cc1cc([C@@H](C)Nc2ccccc2CO)c2oc(-c3ccccc3)c(C)c(=O)c2c1 ; Cc1cc([C@@H](C)Nc2ccccc2CO)c2oc(-c3cccnc3)c(C)c(=O)c2c1 ; Cc1cc([C@@H](C)Nc2ccccc2C(O)C(F)(F)F)c2oc(-c3ccccc3)c(C)c(=O)c2c1 ; Cc1cc([C@@H](C)Nc2ccccc2C(O)C(F)(F)F)c2oc(-c3cccnc3)c(C)c(=O)c2c1] 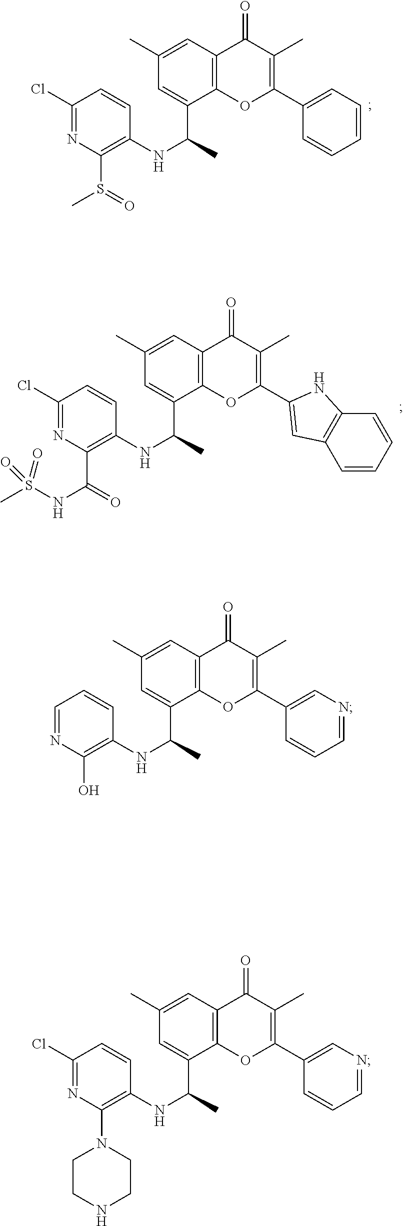 [SMILES: Cc1cc([C@@H](C)Nc2ccc(Cl)nc2S(C)=O)c2oc(-c3ccccc3)c(C)c(=O)c2c1 ; Cc1cc([C@@H](C)Nc2ccc(Cl)nc2C(=O)NS(C)(=O)=O)c2oc(-c3cc4ccccc4[nH]3)c(C)c(=O)c2c1 ; Cc1cc([C@@H](C)Nc2cccnc2O)c2oc(-c3cccnc3)c(C)c(=O)c2c1 ; Cc1cc([C@@H](C)Nc2ccc(Cl)nc2N2CCNCC2)c2oc(-c3cccnc3)c(C)c(=O)c2c1] 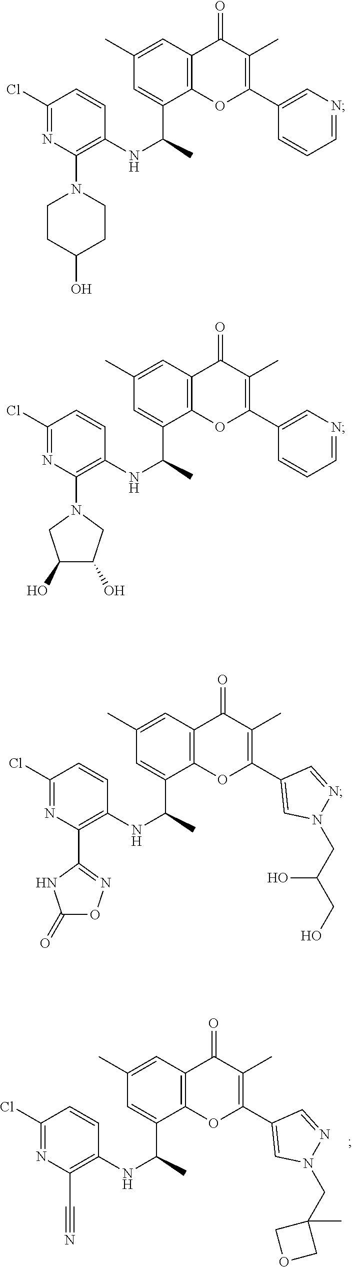 [SMILES: Cc1cc([C@@H](C)Nc2ccc(Cl)nc2N2CCC(O)CC2)c2oc(-c3cccnc3)c(C)c(=O)c2c1 ; Cc1cc([C@@H](C)Nc2ccc(Cl)nc2N2C[C@H](O)[C@@H](O)C2)c2oc(-c3cccnc3)c(C)c(=O)c2c1 ; Cc1cc([C@@H](C)Nc2ccc(Cl)nc2-c2noc(=O)[nH]2)c2oc(-c3cnn(CC(O)CO)c3)c(C)c(=O)c2c1 ; Cc1cc([C@@H](C)Nc2ccc(Cl)nc2C#N)c2oc(-c3cnn(CC4(C)COC4)c3)c(C)c(=O)c2c1] 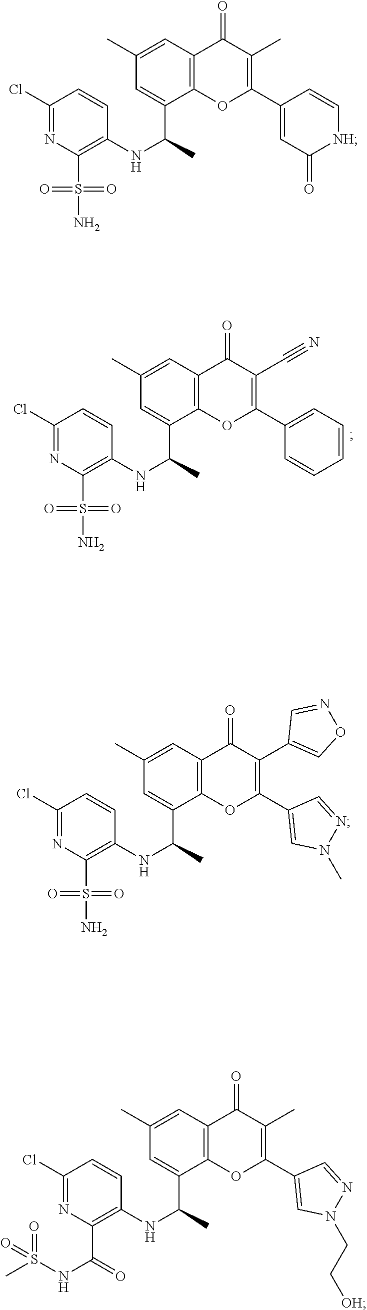 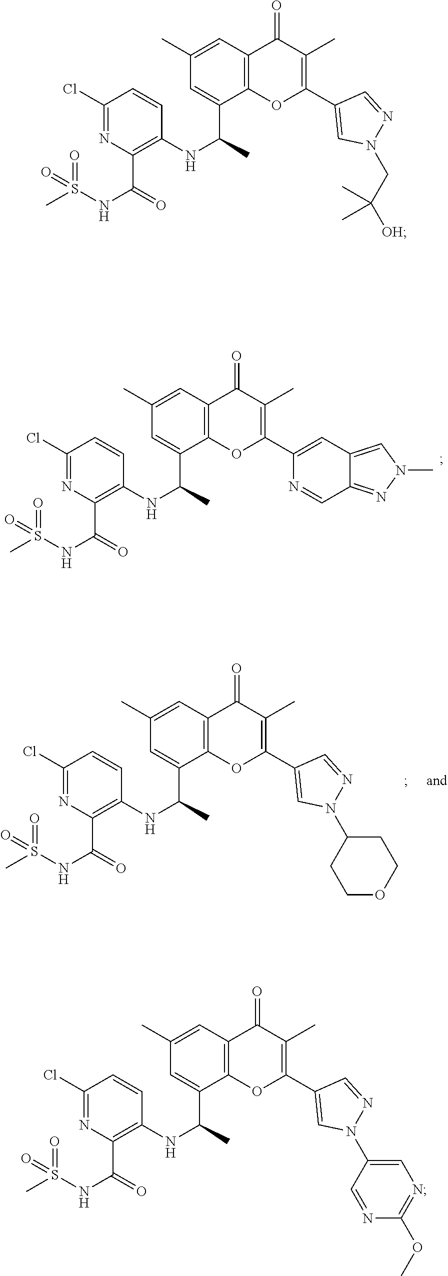
or pharmaceutically acceptable salts thereof.

17. A pharmaceutical composition comprising a compound of claim 1, or a pharmaceutically acceptable salt thereof, and a pharmaceutically acceptable carrier.

18. A method of treating a disease or disorder associated with modulation of phosphoinositide 3-kinase (PI3K), comprising administering to a patient in need thereof a therapeutically effective amount of a compound of claim 1.

19. A method of inhibiting phosphoinositide 3-kinase (PI3K), comprising administering to a patient in need thereof a therapeutically effective amount of a compound of claim 1.

20. A compound of claim 1, or a pharmaceutically acceptable salt thereof for use in therapy.

21. A compound of claim 1, or a pharmaceutically acceptable salt thereof, for use in the treatment of a disease or disorder associated with modulating PI3K.

22. Use of a compound of claim 1, or a pharmaceutically acceptable salt thereof, in the manufacture of a medicament for the treatment of a disease or disorder associated with modulating PI3K.
